(19) United States
(12) Patent Application Publication
(10) Pub. No.: US 2025/0122218 A1
(43) Pub. Date: Apr. 17, 2025

(54) MODULATORS OF CYSTIC FIBROSIS TRANSMEMBRANE CONDUCTANCE REGULATOR

(71) Applicant: Vertex Pharmaceuticals Incorporated, Boston, MA (US)

(72) Inventors: Jeremy J. CLEMENS, San Diego, CA (US); Alexander R. ABELA, Escondido, CA (US); Brett C. BOOKSER, Kensington, CA (US); Thomas CLEVELAND, San Marcos, CA (US); Timothy R. COON, Carlsbad, CA (US); Michel GALLANT, Pierrefonds (CA); Sara Sabina HADIDA RUAH, La Jolla, CA (US); Peter GROOTENHUIS, Del Mar, CA (US); Yoshihiro ISHIHARA, San Diego, CA (US); Julie LATERREUR, San Diego, CA (US); Jason MCCARTNEY, Cardiff by the Sea, CA (US); Mark Thomas MILLER, Rancho Santa Fe, CA (US); Prasuna PARASELLI, San Diego, CA (US); Yeeman K. RAMTOHUL, Pierrefonds (CA); Thumkunta Jagadeeswar REDDY, Pierrefonds (CA); William SCHULZ BECHARA, Laval (CA); Claudio STURINO, Ile Bizard (CA); Joe A. TRAN, San Marcos, CA (US); Lino VALDEZ, San Diego, CA (US); Jinglan ZHOU, San Diego, CA (US); Ramkrishna DE, Vista, CA (US)

(21) Appl. No.: 18/836,499

(22) PCT Filed: Feb. 7, 2023
(86) PCT No.: PCT/US2023/012546

§ 371 Date: Aug. 7, 2024

Related U.S. Application Data
(60) Provisional application No. 63/307,954, filed on Feb. 8, 2022.

(51) Int. Cl. C07D 498/22; A61K 31/404; A61K 31/4155; A61K 31/439; A61K 31/4433; A61K 31/4439; A61K 31/47; A61K 31/529; C07D 498/18

(52) U.S. Cl. CPC C07D 498/22; A61K 31/404; A61K 31/4155; A61K 31/439; A61K 31/4433; A61K 31/4439; A61K 31/47; A61K 31/529; C07D 498/18

(57) ABSTRACT

This disclosure provides modulators of Cystic Fibrosis Transmembrane Conductance Regulator (CFTR), pharmaceutical compositions containing at least one such modulator, methods of treatment of cystic fibrosis using such modulators and pharmaceutical compositions, and processes for making such modulators.

This application claims the benefit of U.S. Provisional Application No. 63/307,954, filed on Feb. 8, 2022, the contents of which are incorporated by reference in its entirety.

The invention relates to modulators of Cystic Fibrosis Transmembrane Conductance Regulator (CFTR), pharmaceutical compositions containing the modulators, methods of treatment of cystic fibrosis and CFTR-mediated disorders using such modulators and pharmaceutical compositions, and processes for making such modulators.

Cystic fibrosis (CF) is a recessive genetic disease that affects approximately 83,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B S. et al. (1989) Science 245:1073-1080; Kerem, B S. et al. (1990) Proc. Natl. Acad. Sci. USA 87:8447-8451). To date, greater than 2000 mutations in the CF gene have been identified; currently, the CFTR2 database contains information on at least 322 of these identified mutations, with sufficient evidence to define at least 281 mutations as disease-causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC (epithelial sodium channel) and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectoral transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride. Because water is probably never actively transported itself, its flow across epithelia depends on tiny transepithelial osmotic gradients generated by the bulk flow of sodium and chloride.

A number of CFTR modulators have recently been identified. These modulators can be characterized as, for example, potentiators, correctors, potentiator enhancers/co-potentiators, amplifiers, readthrough agents, and nucleic acid therapies. CFTR modulators that increase the channel gating activity of mutant and wild-type CFTR at the epithelial cell surface are known as potentiators. Correctors improve faulty protein processing and resulting trafficking to the epithelial surface. Ghelani and Schneider-Futschik (2020) ACS Pharmacol. Transl. Sci. 3:4-10. There are three CFTR correctors approved by the U.S. FDA for treatment of cystic fibrosis. However, monotherapy with some CFTR correctors has not been found to be effective enough and as a result combination therapy with a potentiator is often needed to enhance CFTR activity. There is currently only one CFTR potentiator that is approved for the treatment of cystic fibrosis. Thus, although the treatment of cystic fibrosis has been transformed by these new small molecule CFTR modulators, new and better modulators are needed to prevent disease progression, reduce the severity of the cystic fibrosis and other CFTR-mediated diseases, and to treat the more severe forms of these diseases.

One aspect of the invention provides novel compounds, including compounds of Formulae I, I′, Ia, Ib, Ia′, Ib′, IIa, IIb, IIc, IId, IIe, and IIf, Compounds 1-271, deuterated derivatives thereof and pharmaceutically acceptable salts of any of the foregoing.

For example, compounds of Formula I can be depicted as:

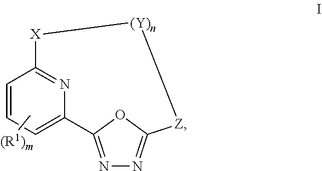

and includes deuterated derivatives and pharmaceutically acceptable salts thereof, wherein:
X is selected from —C(R^{X1})₂—, —CO—,

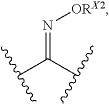

—Si(R^{Z3})₂—, and

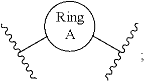

Ring A is a cyclic group selected from phenyl and 5- to 6-membered heteroaryl, wherein the cyclic group is optionally substituted with 1-3 groups independently selected from C₁-C₆ alkyl;
each R^{X1} is independently selected from H, C₁-C₆ alkyl (optionally substituted with 1-3 groups independently selected from oxo, —OR^{X2}, and —N(R^{X2})₂), C₃-C₈ cycloalkyl, halogen, cyano, —OR^{X2}, and C₁-C₆ fluoroalkyl;
each R^{X2} is independently selected from H and C₁-C₆ alkyl;
each Y is independently selected from —C(R^{Y})₂—, —O—, —CO—, —NR^{YN}—, and

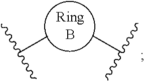

each R^{Y} is independently selected from hydrogen, hydroxy, halogen, C₁-C₆ alkyl (optionally substituted with 1-3 groups independently selected from hydroxy, C₁-C₆ alkoxy, and Q), C₃-C₈ cycloalkyl, C₆-C₁₀ aryl (optionally substituted with 1-3 groups independently selected from halogen), 5- to 10-membered heteroaryl, —OR^{Y1}, —CO₂R^{Y1}, —COR^{Y1}, —CON(R^{Y1})₂, and —N(R^{Y1})₂;
or two R^{Y}, one of which is on one atom and the second of which is on an adjacent atom, are taken together to form a pi bond;
each R^{Y1} is independently selected from hydrogen and C₁-C₆ alkyl, or two R^{Y1} bonded to the same nitrogen taken together form a 3- to 6-membered heterocyclyl;
each R^{YN} is independently selected from:
   H,
   C₁-C₆ alkyl, optionally substituted with 1-3 groups independently selected from:
      oxo,
      C₃-C₈ cycloalkyl (optionally substituted with 1-5 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₈ cycloalkyl (optionally substituted with 1-3 groups independently selected from halogen, C₁-C₆ alkyl, and C₁-C₆ fluoroalkyl), and
         phenyl (optionally substituted with 1-3 groups independently selected from C₁-C₆ alkyl, C₁-C₆ alkoxy, C₁-C₆ fluoroalkyl, C₁-C₆ fluoroalkoxy, and halogen),
      5- to 10-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         —O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN}1 is independently selected from H, C₁-C₄ alkyl (optionally substituted with oxo), and C₃-C₆ cycloalkyl;
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl,
   5- to 10-membered heteroaryl, and
   3- to 6-membered heterocyclyl (optionally substituted with 1-3 groups independently selected from C₁-C₆ alkyl);
each Q is independently selected from:
   C₁-C₆ alkyl optionally substituted with 1-3 groups independently selected from:
      halogen,
      oxo,
      C₆-C₁₀ aryl (optionally substituted with 1-3 groups independently selected from halogen and —OCF₃), and
      C₃-C₈ cycloalkyl,
   C₃-C₈ cycloalkyl optionally substituted with 1-3 groups independently selected from:
      halogen,
      CN,
      C₁-C₆ alkyl (optionally substituted with 1-3 groups independently selected from halogen, —NH₂, and —NHCOMe),
      C₁-C₆ alkoxy,
      C₆-C₁₀ aryl (optionally substituted with 1-3 groups independently selected from C₁-C₆ alkyl), and
      C₃-C₈ cycloalkyl,
   C₆-C₁₀ aryl optionally substituted with 1-3 groups independently selected from:
      halogen,
      CN,
      C₁-C₆ alkyl (optionally substituted with 1-3 groups independently selected from halogen and hydroxy),
      C₁-C₆ alkoxy optionally substituted with 1-4 groups independently selected from:
         halogen,
         C₃-C₈ cycloalkyl (optionally substituted with CF₃),
      C₃-C₈ cycloalkyl (optionally substituted with 1-3 groups independently selected from halogen, CF₃, OCF₃, and C₁-C₆ alkyl), and
      C₆-C₁₀ aryl,
   5- to 10-membered heteroaryl optionally substituted with 1-3 groups independently selected from:
      halogen,
      C₁-C₆ alkyl (optionally substituted with 1-3 groups independently selected from halogen),
      C₃-C₈ cycloalkyl (optionally substituted with 1-3 CF₃ groups), and
      3- to 10-membered heterocyclyl,
   3- to 10-membered heterocyclyl optionally substituted with 1-3 groups independently selected from:
      C₁-C₆ alkyl (optionally substituted with 1-3 groups independently selected from oxo and C₃-C₈ cycloalkyl), and
      oxo;
each R¹ is independently selected from halogen, C₁-C₆ fluoroalkyl, C₁-C₆ alkyl (optionally substituted with a group selected from hydroxy, C₆-C₁₀ aryl, and 5- to 6-membered heteroaryl), —OR², —N(R²)₂, —CO₂R², —CO—N(R²)₂, —CN, C₃-C₈ cycloalkyl, C₆-C₁₀ aryl, 5- to 6-membered heteroaryl (optionally substituted with 1-3 groups independently selected from C₁-C₆ alkyl), 3- to 6-membered heterocyclyl, —B(OR²)₂, —SO₂R², —SR², —SOR², —PO(OR²)₂, and —PO(R²)₂;
each R² is independently selected from hydrogen, C₁-C₆ alkyl (optionally substituted with 1-6 groups independently selected from halogen), C₁-C₆ fluoroalkyl, and C₆-C₁₀ aryl (optionally substituted with 1-3 groups independently selected from C₁-C₆ fluoroalkyl and C₁-C₆ fluoroalkoxy);
Z is selected from

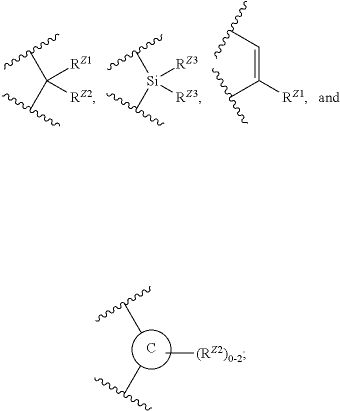

wherein Ring C is selected from C₆-C₁₀ aryl and 5- to 10-membered heteroaryl;
R^{Z1} is selected from hydrogen, —CN, C₁-C₆ alkyl (optionally substituted with 1-3 hydroxy), C₁-C₆ fluoroalkyl, 3- to 6-membered heterocyclyl, C₃-C₆ cycloalkyl, C₆-C₁₀ aryl, and 5- to 6-membered heteroaryl;
R^{Z2} is selected from hydrogen, halogen, hydroxy, NH₂, NH(CO)(C₁-C₆ alkyl), and C₁-C₆ alkoxy (optionally substituted with 1-3 groups independently selected from C₃-C₁₀ cycloalkyl),
or R^{Z1} and R^{Z2} taken together form a group selected from oxo and ═N—OH;
each R^{Z3} is independently selected from hydroxy, C₁-C₆ alkoxy, C₁-C₆ alkyl, and C₆-C₁₀ aryl; or two R^{Z3} are taken together to form a 3- to 6-membered heterocyclyl;
n is selected from 4, 5, 6, 7, and 8; and
m is selected from 0, 1, 2, and 3.

In some embodiments of Formula I, X is —C(R^{X1})₂—. In some embodiments of Formula I, —C(R^{X1})₂— is a group selected from

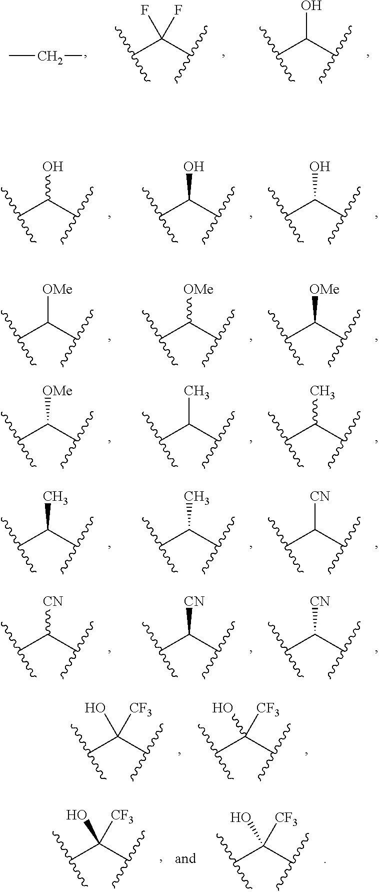

In some embodiments of Formula I, X is —CO—.

In some embodiments of Formula I, X is

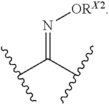

In some embodiments of Formula I, X is a group selected from:

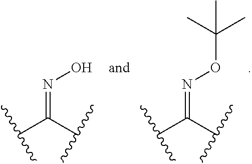

In some embodiments of Formula I, X is

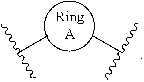

In some embodiments of Formula I, Ring A is a cyclic group selected from phenyl, pyrazole, and oxadiazole.

In some embodiments of Formula I, X is selected from:

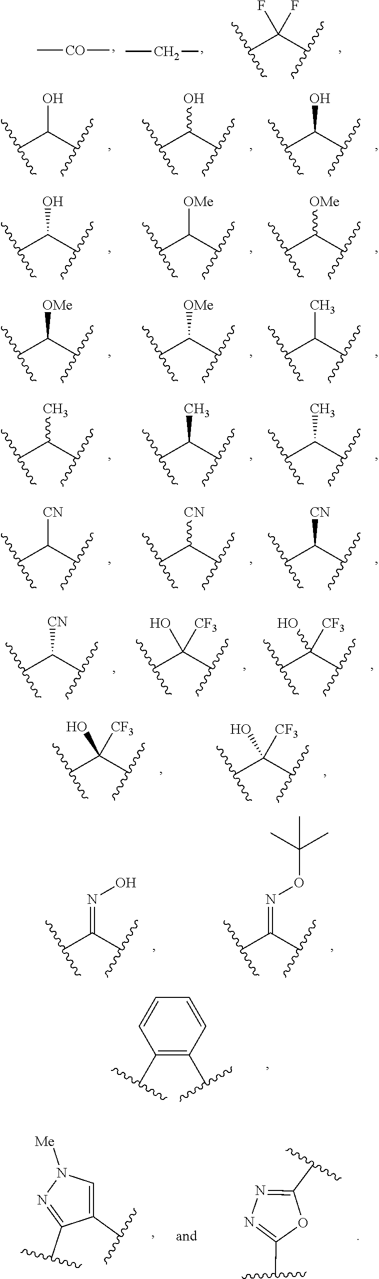

In some embodiments of Formula I, each R^{X1} is independently selected from H, C₁-C₆ alkyl, halogen, cyano, —OR^{X2}, and C₁-C₆ fluoroalkyl. In some embodiments of Formula I, each R^{X2} is independently selected from H and C₁-C₄ alkyl. In some embodiments of Formula I, each R^{X1} is independently selected from H, F, —CF₃, —CH₃, —OH, —OCH₃, and CN.

In some embodiments of Formula I, each Y is independently selected from —C(R^{Y})₂—, —CO—, —NR^{YN}—, and

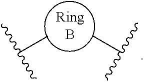

In some embodiments of Formula I, each R^{Y} is independently selected from hydrogen, hydroxy, halogen, and C₁-C₆ alkyl. In some embodiments of Formula I, each R^{Y} is independently selected from H, —OH, —F, and —CH₃.

In some embodiments of Formula I, each R^{YN} is independently selected from:
H,
CH₂CF₃,
C₁-C₆ alkyl, optionally substituted with 1-3 groups independently selected from:
   oxo,
   cyclopropyl,
   cyclobutyl optionally substituted with 1-2 fluoro groups,
   cyclohexyl optionally substituted with 1-2 fluoro groups,
   spiro[2.2]pentane,
   bicyclo[4.1.0]heptane,
   dispiro[2.0.24.13]heptane,
   phenyl optionally substituted with 1-3 groups independently selected from F, Cl, Br, CN, methyl, propyl (optionally substituted with one hydroxy), tert-butyl, neopentyl, —CHF₂, —CF₃, —C(CH₃)₂CF₃, —OCH₃, —O(2-propyl), —OCHF₂, —OCF₃, —OCF₂CHF₂, —OCH₂CF₃, cyclopropyl (optionally substituted with one CF₃), cyclobutyl (optionally substituted with 1-2 groups selected from fluoro and methyl), spiro[2.2]pentane, bicyclo[1.1.1]pentane, bicyclo[4.1.0]heptane, dispiro[2.0.24.13]heptane, phenyl, naphthyl, and

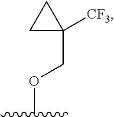 [SMILES: *OCC1(C(F)(F)F)CC1]

naphthyl,
2,2-dimethyl-2,3-dihydrobenzofuran,
2,2-difluorobenzo[d][1,3]dioxole,
tetrahydro-2H-pyran,
pyrazole optionally substituted with one group selected from methyl and propyl,
pyridine optionally substituted with one group selected from methyl, tert-butyl, CF₃, —O(cyclobutyl), —OCHF₂, cyclopropyl optionally substituted with one CF₃, and piperidine, and
pyrimidine optionally substituted with one group selected from methyl and tert-butyl,
cyclopropyl optionally substituted with 1-2 groups selected from methyl, tert-butyl, cyclopropyl, and phenyl (optionally substituted with one tert-butyl group),
cyclobutyl optionally substituted with 1-3 groups selected from fluoro, methyl, and phenyl,
cyclopentyl,
cyclohexyl optionally substituted with 1-2 groups selected from fluoro, methyl, tert-butyl, cyano, —CF₃, —CH₂NH₂, and —CH₂NHAc,
bicyclo[1.1.1]pentane optionally substituted by one group selected from fluoro, methyl, and tert-butyl,
bicyclo[2.2.1]heptane optionally substituted with one —OCH₃ group,
bicyclo[2.2.2]octane,
spiro[3.3]heptane optionally substituted with 1-2 fluoro groups,
phenyl,
tetrahydro-2H-pyran,
2-azaspiro[3.3]heptane optionally substituted with one group selected from —COMe, and —CO(cyclopropyl),
thietane 1,1-dioxide,
tetrahydro-2H-thiopyran 1,1-dioxide,
pyrazole optionally substituted with one methyl group, and
pyridine.

In some embodiments of Formula I, each R^{YN} is independently selected from: H, —CH₃,

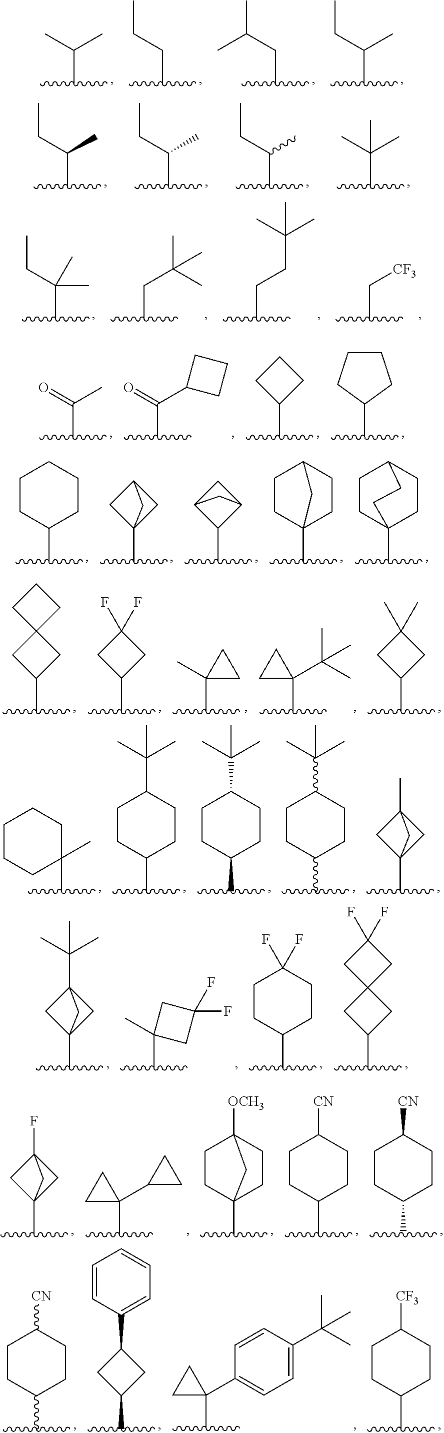

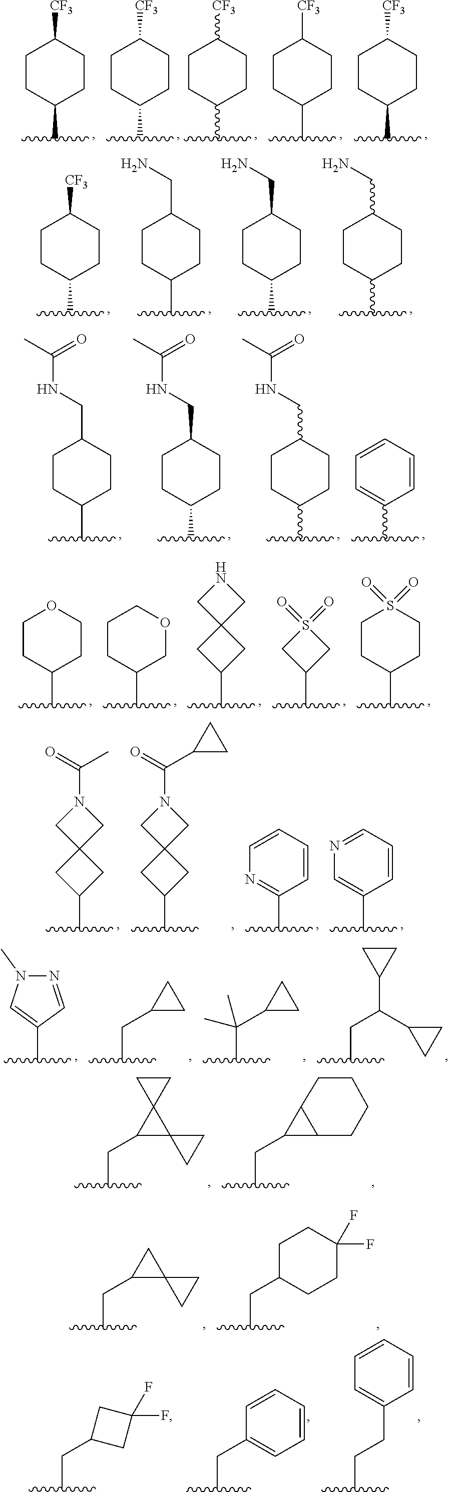

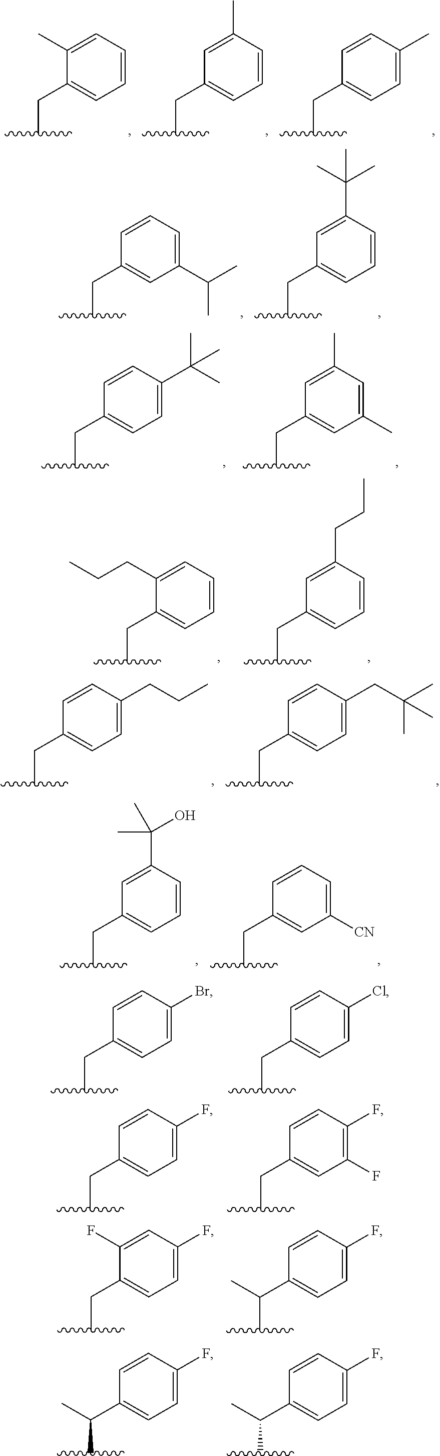

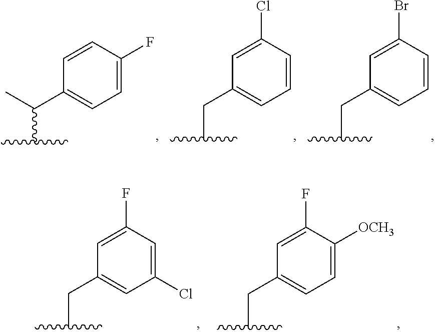

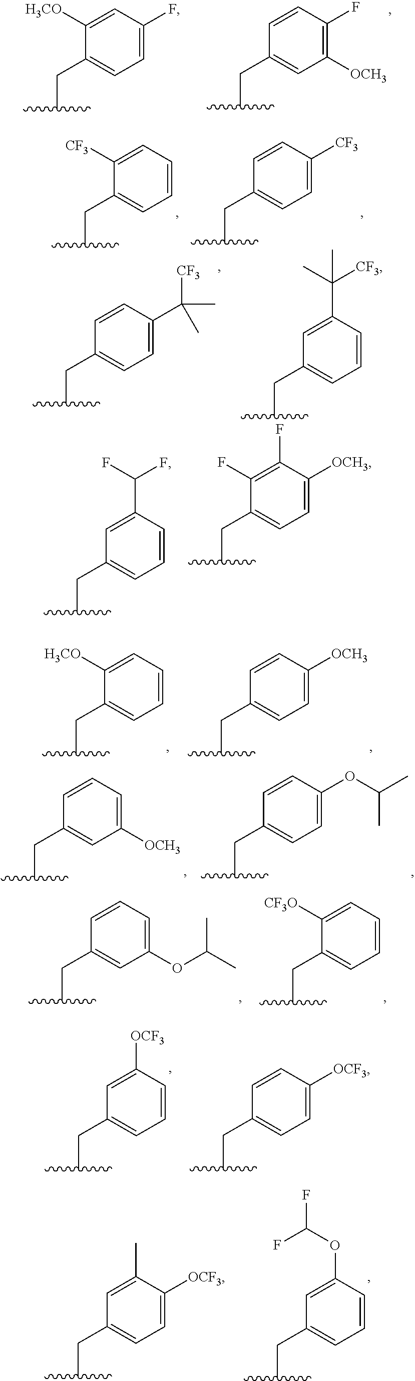

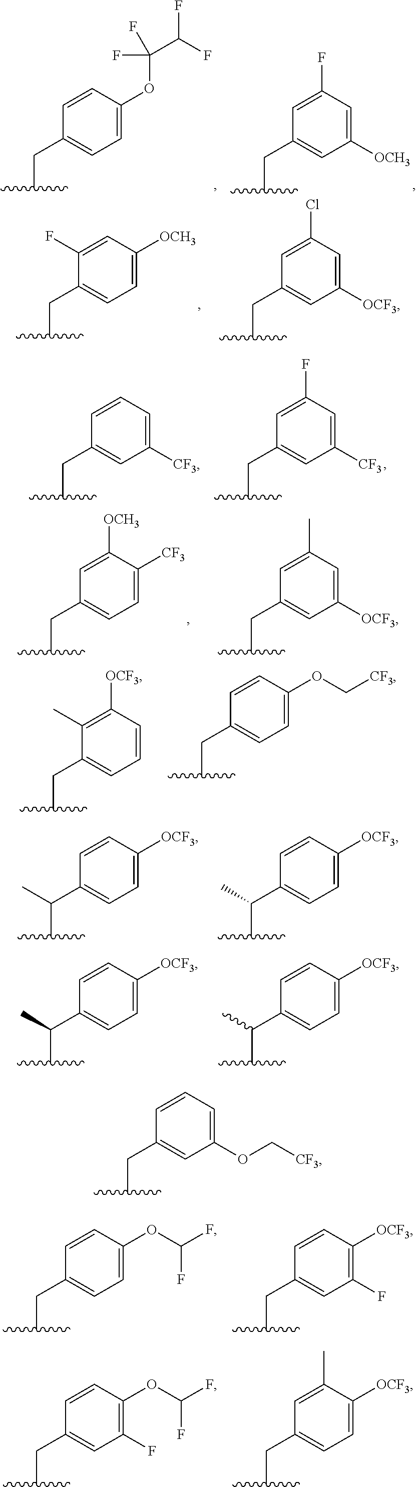

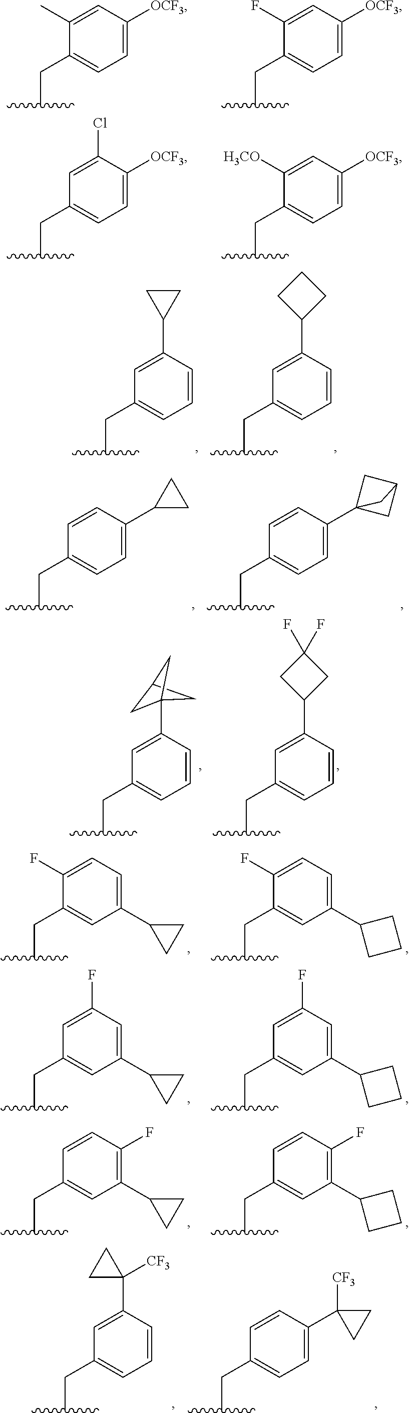

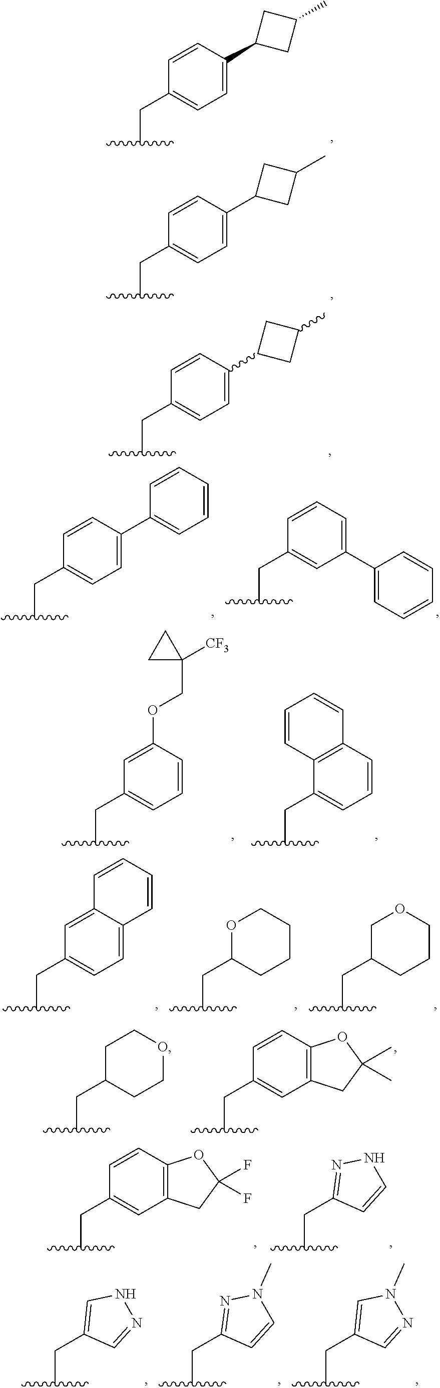

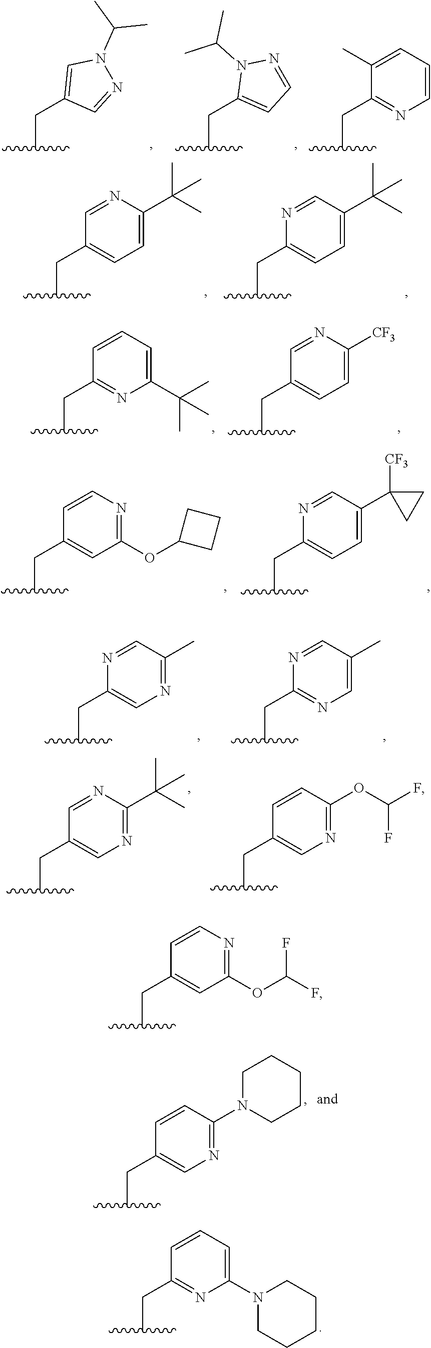

In some embodiments of Formula I, Ring B is selected from:
C₆-C₁₀ aryl (optionally substituted with 1-3 groups independently selected from halogen),
C₃-C₈ cycloalkyl, and
3- to 6-membered heterocyclyl (optionally substituted with 1-3 groups independently selected from C₁-C₆ alkyl).

In some embodiments of Formula I, Ring B is selected from:

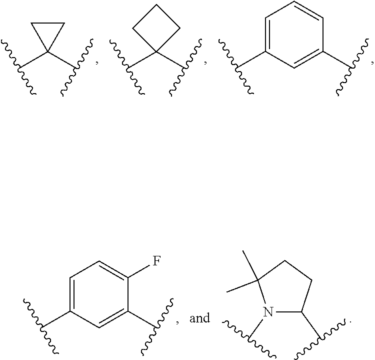

In some embodiments of Formula I, each R² is H.

In some embodiments of Formula I, each R¹ is independently selected from C₁-C₆ fluoroalkyl, —N(R²)₂, and —CN. In some embodiments of Formula I, each R¹ is independently selected from —CF₃, —NH₂, and —CN.

In some embodiments of Formula I, Z is selected from

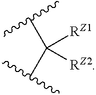

In some embodiments of Formula I, Z is selected from:

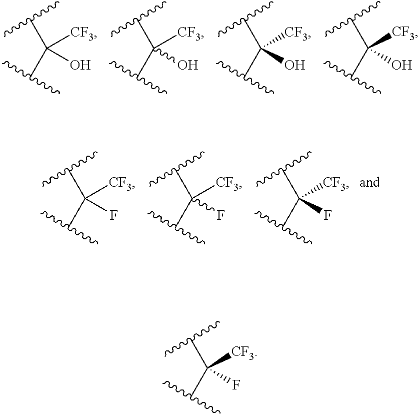

In some embodiments of Formula I, Z is

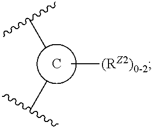

wherein Ring C is selected from C₆-C₁₀ aryl. In some embodiments of Formula I, the group:

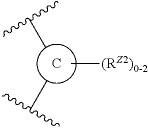

is selected from:

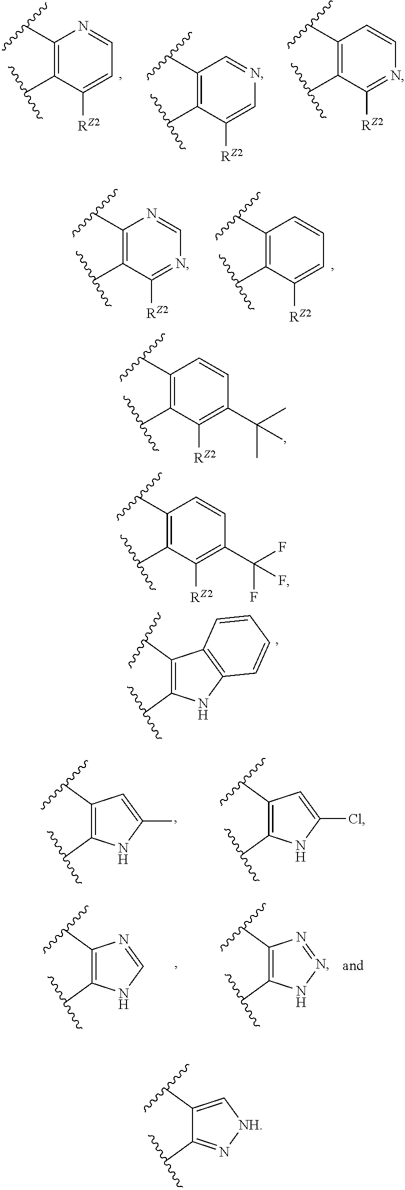

In some embodiments of Formula I, the group:

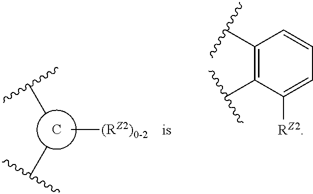

In some embodiments of Formula I, Z is

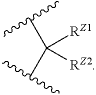

In some embodiments of Formula I, Z is

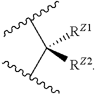

In some embodiments of Formula I, Z is

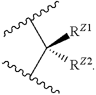

In some embodiments of Formula I, Z is

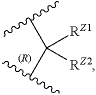

wherein (R) refers to the stereochemical designation of the central carbon atom under the Cahn-Ingold-Prelog convention. In some embodiments of Formula I, Z is

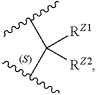

wherein (S) refers to the stereochemical designation of the central carbon atom under the Cahn-Ingold-Prelog convention.

In some embodiments of Formula I, R^{Z1} is selected from C₁-C₆ fluoroalkyl.

In some embodiments of Formula I, R^{Z2} is selected from halogen and hydroxy.

In some embodiments of Formula I, n is selected from 5, 6, and 7.

In some embodiments of Formula I, m is selected from 1 and 2.

In some embodiments, compounds of Formula I can be depicted as Formula I′:

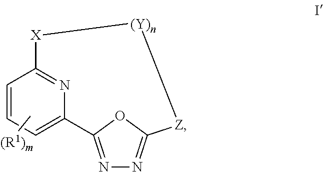

and includes deuterated derivatives and pharmaceutically acceptable salts thereof, wherein:
X is selected from —C(R^{X1})₂—, —CO—,

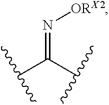

—Si(R^{Z3})₂—, and

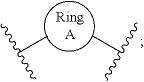

Ring A is a cyclic group selected from phenyl and 5- to 6-membered heteroaryl, wherein the cyclic group is optionally substituted with 1-3 groups independently selected from C₁-C₆ alkyl;
each R^{X1} is independently selected from H, C₁-C₆ alkyl (optionally substituted with 1-3 groups independently selected from oxo, —OR^{X2}, and —N(R^{X2})₂), C₃-C₈ cycloalkyl, halogen, cyano, —OR^{X2}, and C₁-C₆ fluoroalkyl;
each R^{X2} is independently selected from H and C₁-C₆ alkyl;
each Y is independently selected from —C(R^{Y})₂—, —O—, —CO—, —NR^{YN}—, and

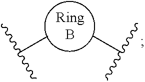

each R^{Y} is independently selected from hydrogen, hydroxy, halogen, C₁-C₆ alkyl (optionally substituted with 1-3 groups independently selected from hydroxy, C₁-C₆ alkoxy, and Q), C₃-C₈ cycloalkyl, C₆-C₁₀ aryl (optionally substituted with 1-3 groups independently selected from halogen), 5- to 10-membered heteroaryl, —OR^{Y1}, —CO₂R^{Y1}, —COR^{Y1}, —CON(R^{Y1})₂, and —N(R^{Y1})₂;
or two R^{Y}, one of which is on one atom and the second of which is on an adjacent atom, are taken together to form a pi bond;
each R^{Y1} is independently selected from hydrogen and C₁-C₆ alkyl, or two R bonded to the same nitrogen taken together form a 3- to 6-membered heterocyclyl;
each R^{YN} is independently selected from:
   H,
   C₁-C₆ alkyl, optionally substituted with 1-3 groups independently selected from:
      oxo,
      C₃-C₈ cycloalkyl (optionally substituted with 1-3 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₈ cycloalkyl (optionally substituted with 1-3 groups independently selected from halogen, C₁-C₆ alkyl, and C₁-C₆ fluoroalkyl), and
         phenyl (optionally substituted with C₁-C₆ alkyl),
      5- to 10-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         —O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN1} is independently selected from H, C₁-C₄ alkyl (optionally substituted with oxo), and C₃-C₆ cycloalkyl;
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl,
   5- to 10-membered heteroaryl, and
   3- to 6-membered heterocyclyl (optionally substituted with 1-3 groups independently selected from C₁-C₆ alkyl);
each Q is independently selected from:
   C₁-C₆ alkyl optionally substituted with 1-3 groups independently selected from:
      halogen,
      oxo,
      C₆-C₁₀ aryl (optionally substituted with 1-3 groups independently selected from halogen and —OCF₃), and
      C₃-C₈ cycloalkyl,
   C₃-C₈ cycloalkyl optionally substituted with 1-3 groups independently selected from:
      halogen,
      CN,
      C₁-C₆ alkyl (optionally substituted with 1-3 groups independently selected from halogen, —NH₂, and —NHCOMe),
      C₁-C₆ alkoxy,
      C₆-C₁₀ aryl (optionally substituted with 1-3 groups independently selected from C₁-C₆ alkyl), and
      C₃-C₈ cycloalkyl,
   C₆-C₁₀ aryl optionally substituted with 1-3 groups independently selected from:
      halogen,
      CN,
      C₁-C₆ alkyl (optionally substituted with 1-3 groups independently selected from halogen and hydroxy),
      C₁-C₆ alkoxy optionally substituted with 1-4 groups independently selected from:
         halogen,
         C₃-C₈ cycloalkyl (optionally substituted with CF₃),
      C₃-C₈ cycloalkyl (optionally substituted with 1-3 groups independently selected from halogen, CF₃, OCF₃, and C₁-C₆ alkyl), and
      C₆-C₁₀ aryl,
   5- to 10-membered heteroaryl optionally substituted with 1-3 groups independently selected from:
      halogen,
      C₁-C₆ alkyl (optionally substituted with 1-3 groups independently selected from halogen),
      C₃-C₈ cycloalkyl (optionally substituted with 1-3 CF₃ groups), and
      3- to 10-membered heterocyclyl,
   3- to 10-membered heterocyclyl optionally substituted with 1-3 groups independently selected from:
      C₁-C₆ alkyl (optionally substituted with 1-3 groups independently selected from oxo and C₃-C₈ cycloalkyl), and
      oxo;
each R¹ is independently selected from halogen, C₁-C₆ fluoroalkyl, C₁-C₆ alkyl (optionally substituted with a group selected from hydroxy, C₆-C₁₀ aryl, and 5- to 6-membered heteroaryl), —OR², —N(R²)₂, —CO₂R², —CO—N(R²)₂, —CN, C₃-C₈ cycloalkyl, C₆-C₁₀ aryl, 5- to 6-membered heteroaryl (optionally substituted with 1-3 groups independently selected from C₁-C₆ alkyl), 3- to 6-membered heterocyclyl, —B(OR²)₂, —SO₂R², —SR², —SOR², —PO(OR²)₂, and —PO(R²)₂;
each R² is independently selected from hydrogen, C₁-C₆ alkyl (optionally substituted with 1-6 groups independently selected from halogen), C₁-C₆ fluoroalkyl, and C₆-C₁₀ aryl (optionally substituted with 1-3 groups independently selected from C₁-C₆ fluoroalkyl and C₁-C₆ fluoroalkoxy);
Z is selected from

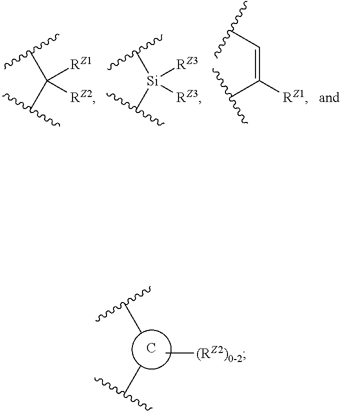

wherein Ring C is selected from C₆-C₁₀ aryl and 5- to 10-membered heteroaryl;
R^{Z1} is selected from hydrogen, —CN, C₁-C₆ alkyl (optionally substituted with 1-3 hydroxy), C₁-C₆ fluoroalkyl, 3- to 6-membered heterocyclyl, C₃-C₆ cycloalkyl, C₆-C₁₀ aryl, and 5- to 6-membered heteroaryl;
R^{Z2} is selected from hydrogen, halogen, hydroxy, NH₂, NH(CO)(C₁-C₆ alkyl), and C₁-C₆ alkoxy (optionally substituted with 1-3 groups independently selected from C₃-C₁₀ cycloalkyl),
or R^{Z1} and R^{Z2} taken together form a group selected from oxo and ═N—OH;
each R^{Z3} is independently selected from hydroxy, C₁-C₆ alkoxy, C₁-C₆ alkyl, and C₆-C₁₀ aryl; or two R^{Z3} are taken together to form a 3- to 6-membered heterocyclyl;
n is selected from 4, 5, 6, 7, and 8; and
m is selected from 0, 1, 2, and 3.

In some embodiments of Formula I′, X is —C(R^{X1})₂—. In some embodiments of Formula I′, —C(R^{X1})₂— is a group selected from —CH₂—,

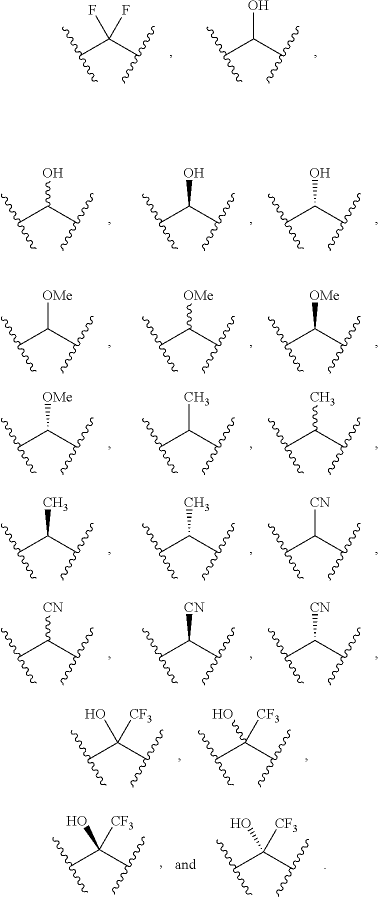

In some embodiments of Formula I′, X is —CO—.

In some embodiments of Formula I′, X is

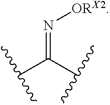

In some embodiments of Formula I′, X is a group selected from:

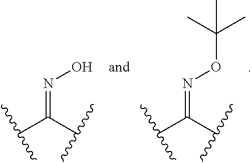

In some embodiments of Formula I′, X is

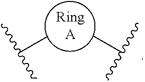

In some embodiments of Formula I′, Ring A is a cyclic group selected from phenyl, pyrazole, and oxadiazole.

In some embodiments of Formula I′, X is selected from: —CO—, —CH₂—,

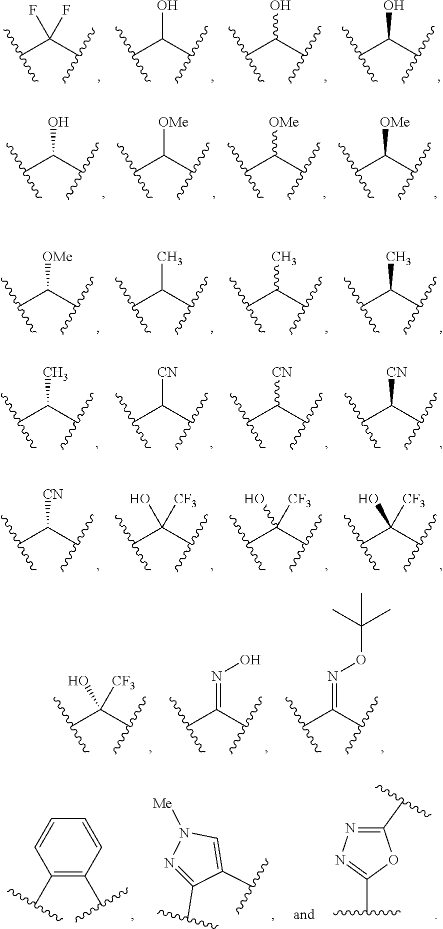

In some embodiments of Formula I′, each R^{X1} is independently selected from H, C₁-C₆ alkyl, halogen, cyano, —OR^{X2}, and C₁-C₆ fluoroalkyl. In some embodiments of Formula I′, each R^{X2} is independently selected from H and C₁-C₄ alkyl. In some embodiments of Formula I′, each R^{X2} is independently selected from H, F, —CF₃, —CH₃, —OH, —OCH₃, and CN.

In some embodiments of Formula I′, each Y is independently selected from —C(R^{Y})₂—, —CO—, —NR^{YN}—, and

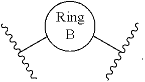

In some embodiments of Formula I′, each R^{Y} is independently selected from hydrogen, hydroxy, halogen, and C₁-C₆ alkyl. In some embodiments of Formula I′, each R^{Y} is independently selected from H, —OH, —F, and —CH₃.

In some embodiments of Formula I′, each R^{YN} is independently selected from:
H,
CH₂CF₃,
C₁-C₆ alkyl, optionally substituted with 1-3 groups independently selected from:
   oxo,
   cyclopropyl,
   cyclobutyl optionally substituted with 1-2 fluoro groups,
   cyclohexyl optionally substituted with 1-2 fluoro groups,
   spiro[2.2]pentane,
   bicyclo[4.1.0]heptane,
   dispiro[2.0.24.13]heptane,
   phenyl optionally substituted with 1-3 groups independently selected from F, Cl, Br, CN, methyl, propyl (optionally substituted with one hydroxy), tert-butyl, neopentyl, —CHF₂, —CF₃, —C(CH₃)₂CF₃, —OCH₃, —O(2-propyl), —OCHF₂, —OCF₃, —OCF₂CHF₂, —OCH₂CF₃, cyclopropyl (optionally substituted with one CF₃), cyclobutyl (optionally substituted with 1-2 groups selected from fluoro and methyl), spiro[2.2]pentane, bicyclo[1.1.1]pentane, bicyclo[4.1.0]heptane, dispiro[2.0.24.13]heptane, phenyl, naphthyl, and

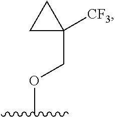 [SMILES: *OCC1(C(F)(F)F)CC1]

naphthyl,
2,2-dimethyl-2,3-dihydrobenzofuran,
2,2-difluorobenzo[d][1,3]dioxole,
tetrahydro-2H-pyran,
pyrazole optionally substituted with one group selected from methyl and propyl,
pyridine optionally substituted with one group selected from methyl, tert-butyl, CF₃, —O(cyclobutyl), —OCHF₂, cyclopropyl optionally substituted with one CF₃, and piperidine, and
pyrimidine optionally substituted with one group selected from methyl and tert-butyl,
cyclopropyl optionally substituted with 1-2 groups selected from methyl, tert-butyl, cyclopropyl, and phenyl (optionally substituted with one tert-butyl group),
cyclobutyl optionally substituted with 1-3 groups selected from fluoro, methyl, and phenyl,
cyclopentyl,
cyclohexyl optionally substituted with 1-2 groups selected from fluoro, methyl, tert-butyl, cyano, —CF₃, —CH₂NH₂, and —CH₂NHAc,
bicyclo[1.1.1]pentane optionally substituted by one group selected from fluoro, methyl, and tert-butyl,
bicyclo[2.2.1]heptane optionally substituted with one —OCH₃ group,
bicyclo[2.2.2]octane,
spiro[3.3]heptane optionally substituted with 1-2 fluoro groups,
phenyl,
tetrahydro-2H-pyran,
2-azaspiro[3.3]heptane optionally substituted with one group selected from —COMe, and —CO(cyclopropyl),
thietane 1,1-dioxide,
tetrahydro-2H-thiopyran 1,1-dioxide,
pyrazole optionally substituted with one methyl group, and
pyridine.

In some embodiments of Formula I′, each R^{YN} is independently selected from: H, —CH₃,

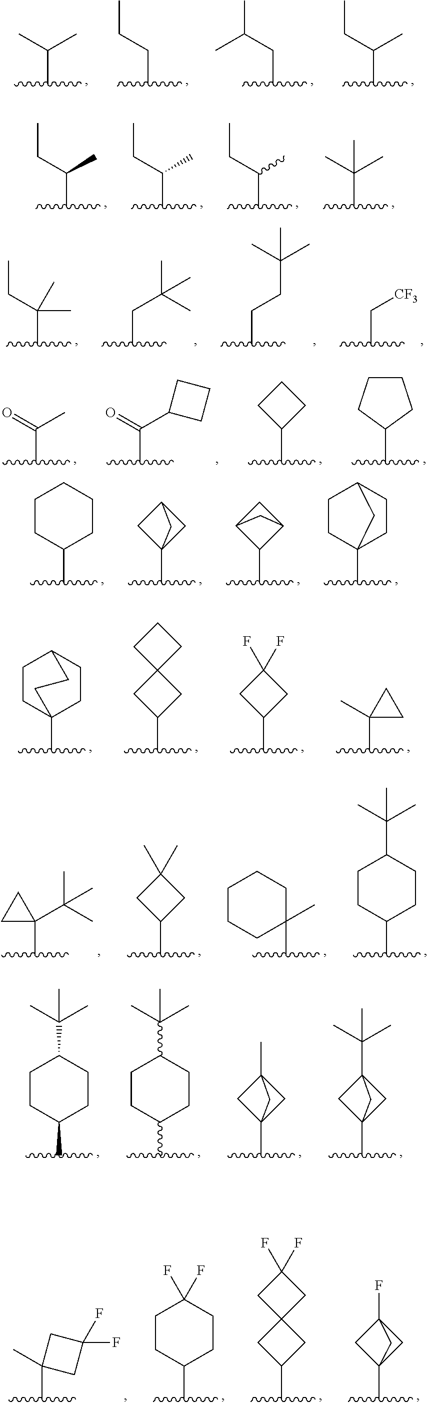

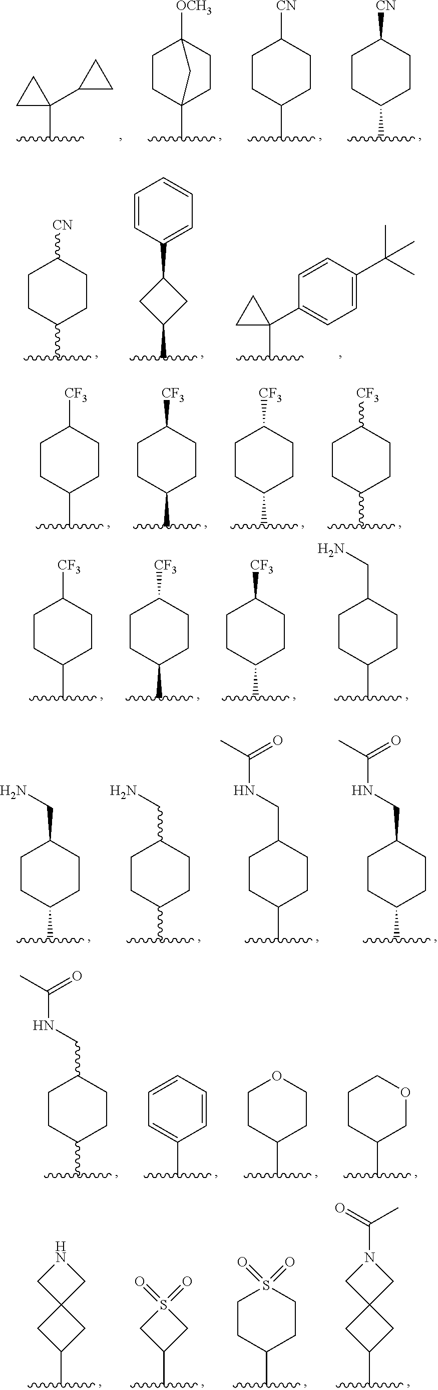

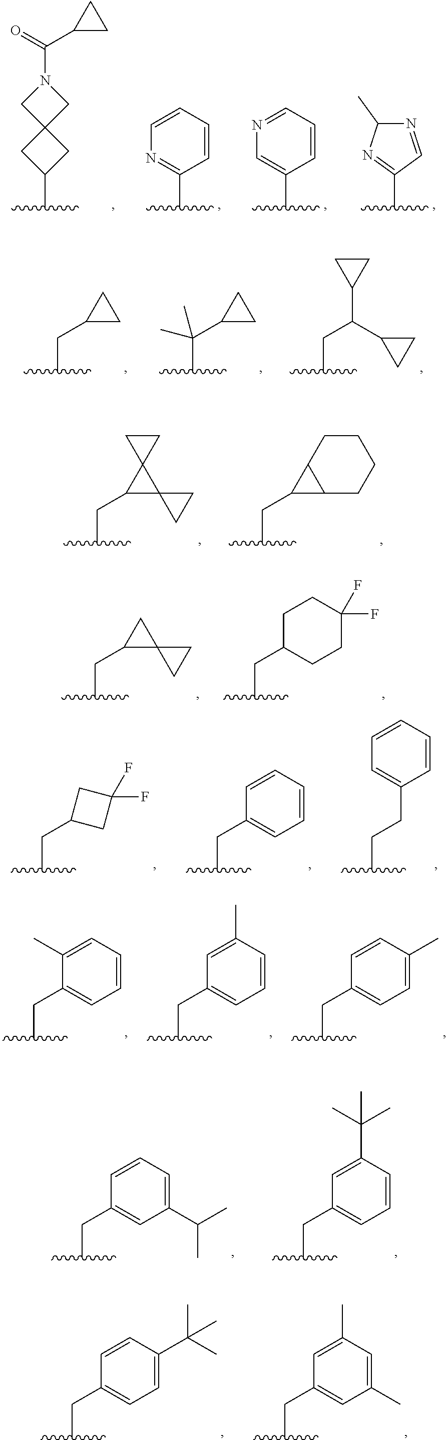

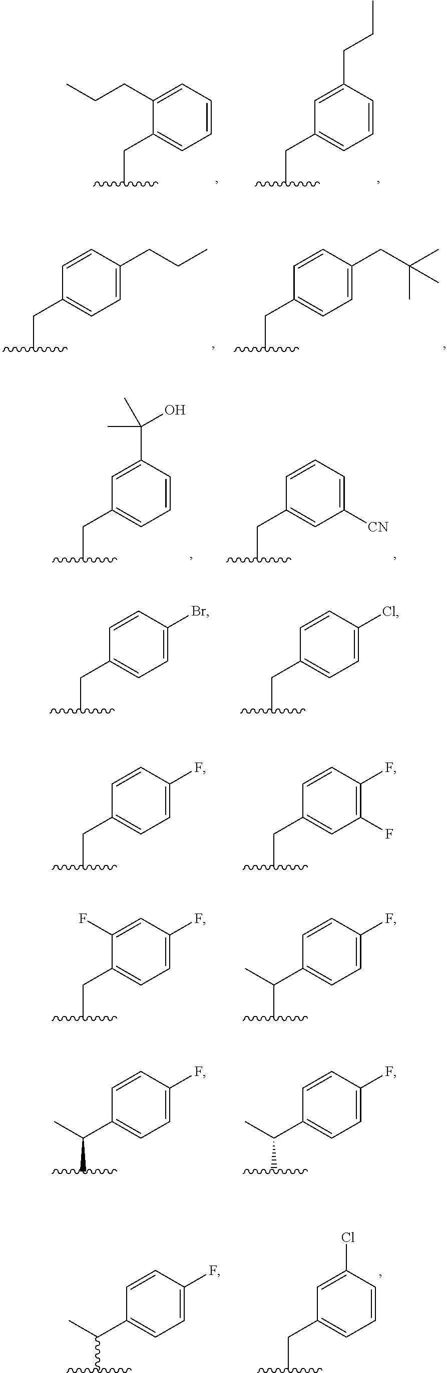

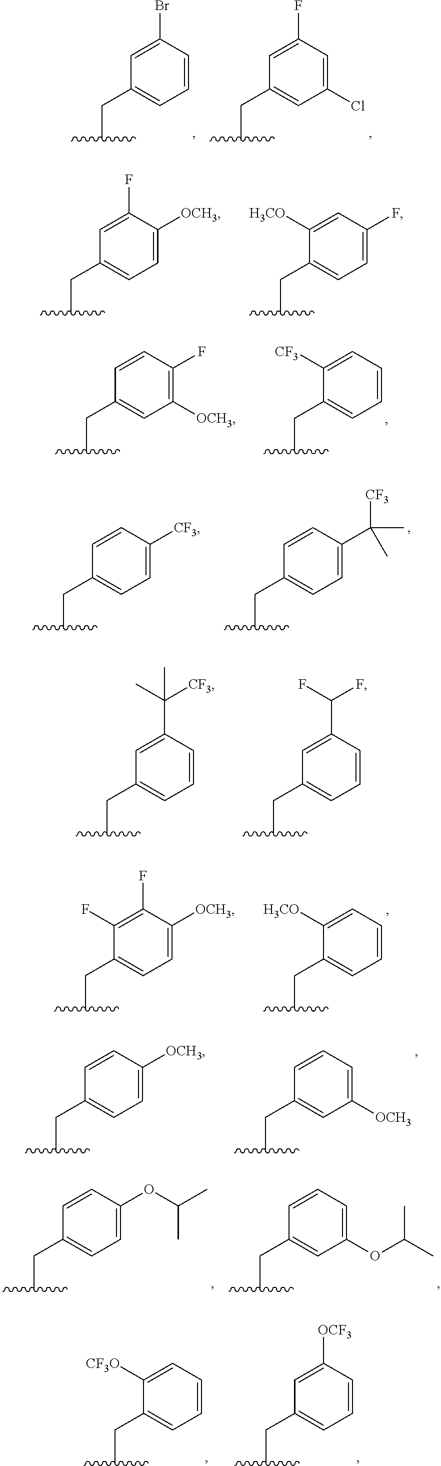

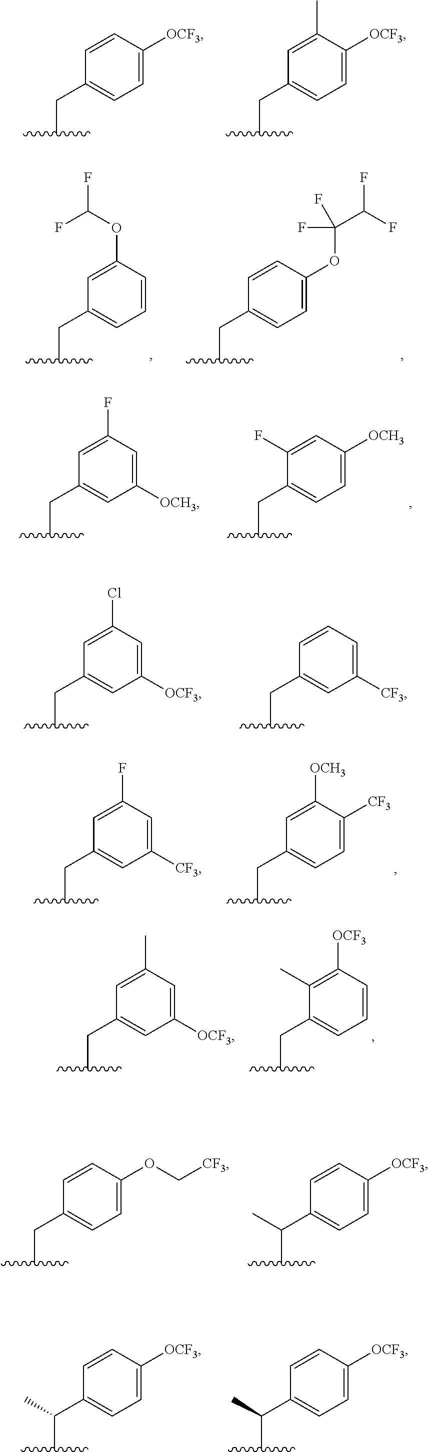

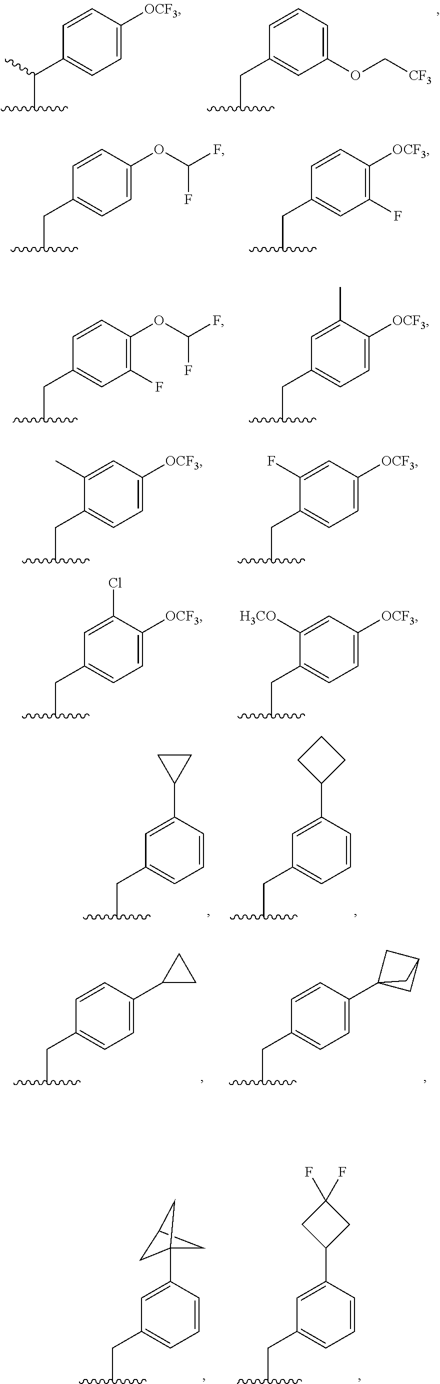

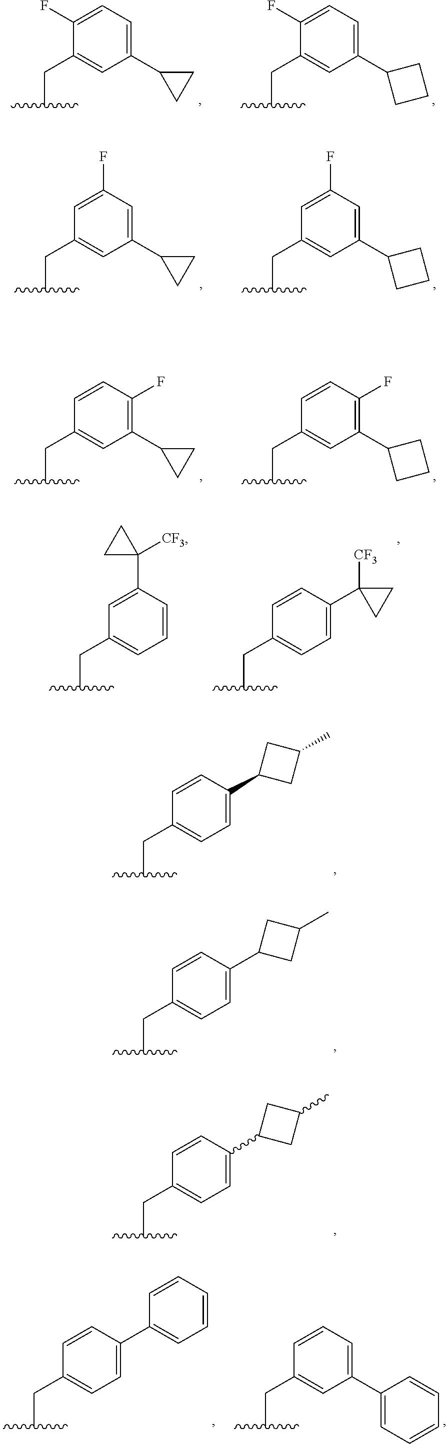

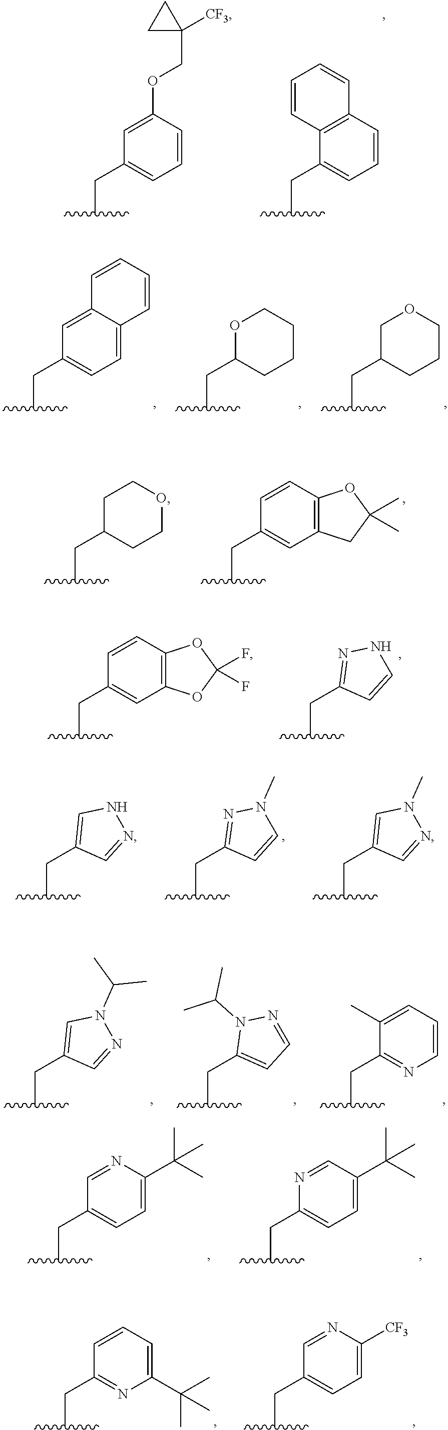

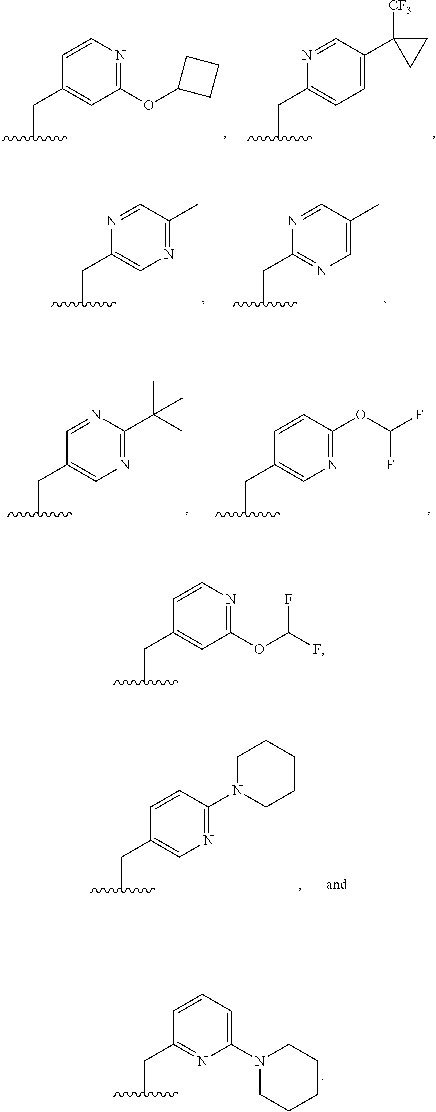

In some embodiments of Formula I′, Ring B is selected from:
C₆-C₁₀ aryl (optionally substituted with 1-3 groups independently selected from halogen),
C₃-C₈ cycloalkyl, and
3- to 6-membered heterocyclyl (optionally substituted with 1-3 groups independently selected from C₁-C₆ alkyl).

In some embodiments of Formula I′, Ring B is selected from:

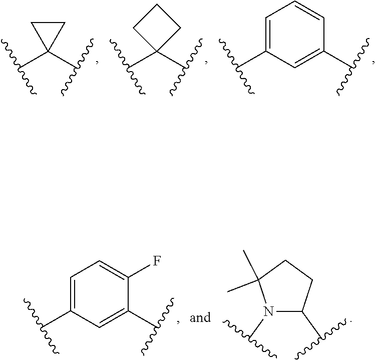

In some embodiments of Formula I′, each R² is H.

In some embodiments of Formula I′, each R¹ is independently selected from C₁-C₆ fluoroalkyl, —N(R²)₂, and —CN. In some embodiments of Formula I′, each R¹ is independently selected from —CF₃, —NH₂, and —CN.

In some embodiments of Formula I′, Z is selected from

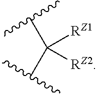

In some embodiments of Formula I′, Z is selected from:

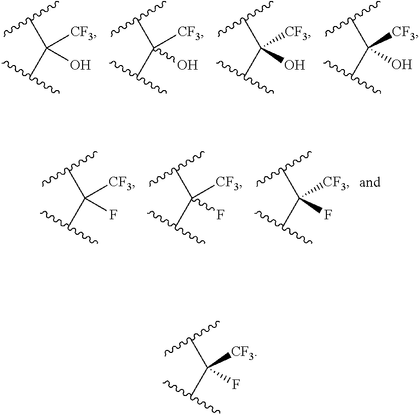

In some embodiments of Formula I′, Z is

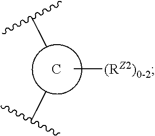

wherein Ring C is selected from C₆-C₁₀ aryl. In some embodiments of Formula I′, the group:

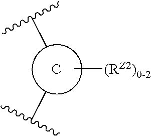

is selected from:

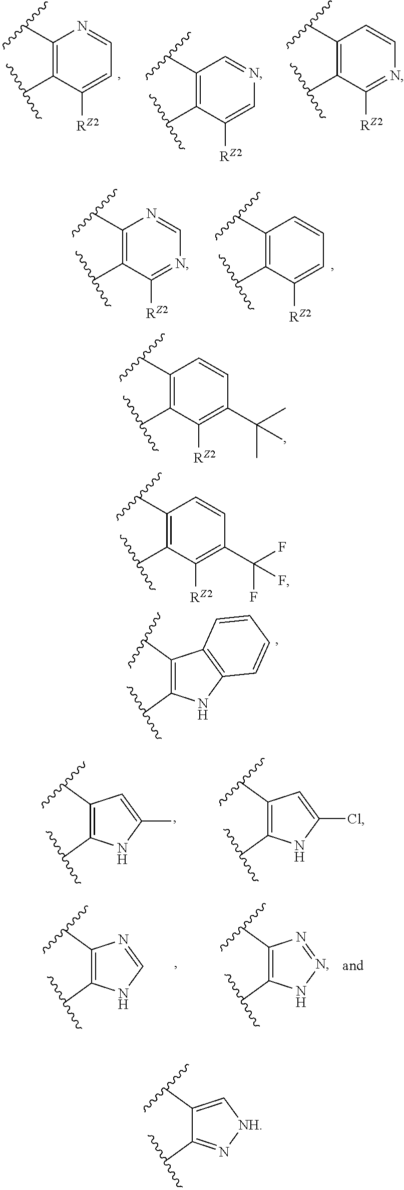

In some embodiments of Formula I′, the group:

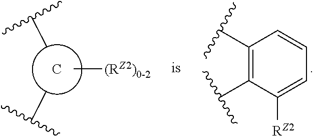

In some embodiments of Formula I′, Z is

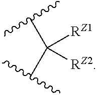

In some embodiments of Formula I′, Z is

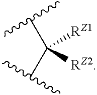

In some embodiments of Formula I′, Z is

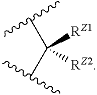

In some embodiments of Formula I′, Z is

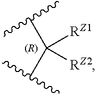

wherein (R) refers to the stereochemical designation of the central carbon atom under the Cahn-Ingold-Prelog convention. In some embodiments of Formula I′, Z is

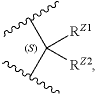

wherein (S) refers to the stereochemical designation of the central carbon atom under the Cahn-Ingold-Prelog convention.

In some embodiments of Formula I′, R^{Z1} is selected from C₁-C₆ fluoroalkyl.

In some embodiments of Formula I′, R^{Z2} is selected from halogen and hydroxy.

In some embodiments of Formula I′, n is selected from 5, 6, and 7.

In some embodiments of Formula I′, m is selected from 1 and 2.

The compounds of the invention also include compounds of Formulae Ia and Ib:

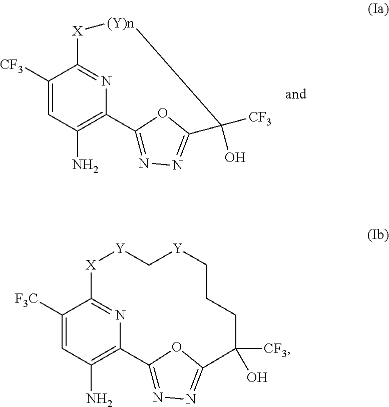

and deuterated derivatives and pharmaceutically acceptable salts thereof, wherein all variables are as defined for Formula I.

In some embodiments of Formulae Ia and Ib, the portion of the compound represented by:

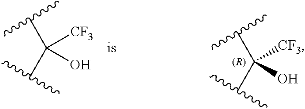

wherein (R) refers to the stereochemical designation of the central carbon atom under the Cahn-Ingold-Prelog convention. In some embodiments of Formula I, the portion of the compound represented by

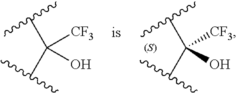

wherein (S) refers to the stereochemical designation of the central carbon atom under the Cahn-Ingold-Prelog convention.

The compounds of the invention also include compounds of Formulae Ia′ and Ib′:

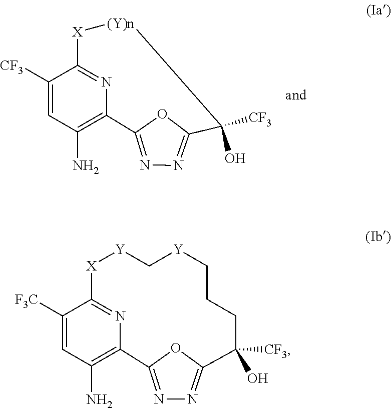

and deuterated derivatives and pharmaceutically acceptable salts thereof, wherein all variables are as defined for Formula I.

In some embodiments of Formulae Ia′ and Ib′, the portion of the compound represented by:

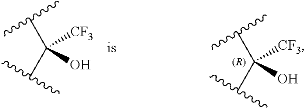

wherein (R) refers to the stereochemical designation of the central carbon atom under the Cahn-Ingold-Prelog convention. In some embodiments of Formula I, the portion of the compound represented by

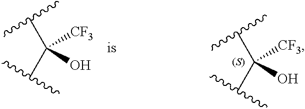

wherein (S) refers to the stereochemical designation of the central carbon atom under the Cahn-Ingold-Prelog convention.

The compounds of the invention also include compounds of Formulae IIa, IIb, IIc, IId, IIe, and IIf:

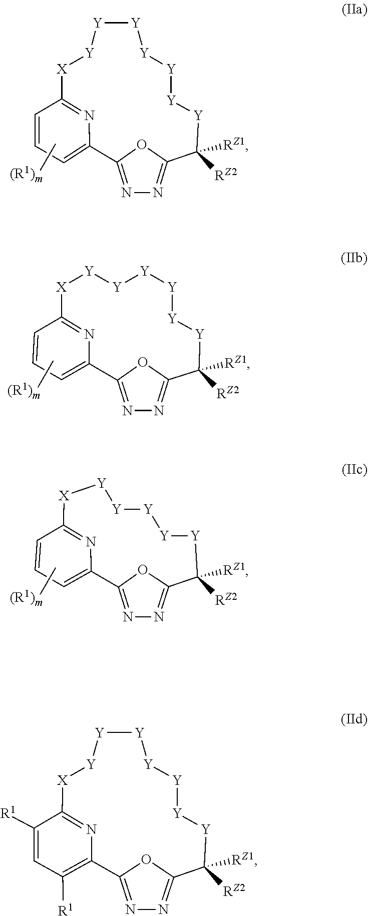

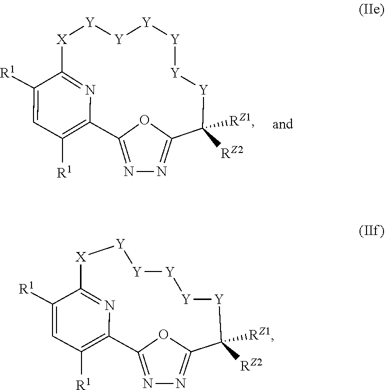

and deuterated derivatives and pharmaceutically acceptable salts thereof, wherein all variables are as defined for Formula I.

In some embodiments of Formulae IIa, IIb, IIc, IId, IIe, and IIf, the portion of the compound represented by:

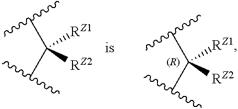

wherein (R) refers to the stereochemical designation of the central carbon atom under the Cahn-Ingold-Prelog convention. In some embodiments of Formula I″, the portion of the compound represented by

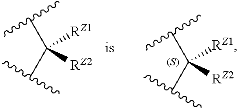

wherein (S) refers to the stereochemical designation of the central carbon atom under the Cahn-Ingold-Prelog convention.

Another aspect of the invention provides pharmaceutical compositions comprising at least one compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, and at least one pharmaceutically acceptable carrier, which compositions may further include at least one additional active pharmaceutical ingredient. Thus, another aspect of the invention provides methods of treating the CFTR-mediated disease cystic fibrosis comprising administering at least one of compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, and at least one pharmaceutically acceptable carrier, optionally as part of a pharmaceutical composition comprising at least one additional component, to a subject in need thereof.

In certain embodiments, the pharmaceutical compositions of the invention comprise at least one compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing. In some embodiments, compositions comprising at least one compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing may optionally further comprise at least one compound chosen from Compound II, Compound IV, Compound V, Compound VI, Compound VII, Compound VIII, Compound IX, Compound X, deuterated derivatives thereof, and pharmaceutically acceptable salts of any of the foregoing.

Another aspect of the invention provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents selected from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II), N-[2,4-bis(1,1-dimethylethyl)-5-hydroxyphenyl]-1,4-dihydro-4-oxoquinoline-3-carboxamide (Compound III) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound III-d), 3-(6-(1-(2,2-difluorobenzo[d][1,3]dioxol-5-yl)cyclopropane carboxamido)-3-methylpyridin-2-yl)benzoic acid (Compound IV), N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound V), N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide (Compound VI), (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound VII), (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound VIII); N-(benzenesulfonyl)-6-(3-fluoro-5-isobutoxy-phenyl)-2-[(4S)-2,2,4-trimethylpyrrolidin-1-yl]pyridine-3-carboxamide (Compound IX), and N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide (Compound X).

Another aspect of the invention provides methods of treating the CFTR-mediated disease cystic fibrosis comprising administering to a patient in need thereof at least one compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, and optionally further administering one or more additional CFTR modulating agents selected from:

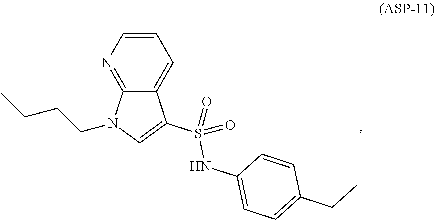 [SMILES: CCCCn1cc(S(=O)(=O)Nc2ccc(CC)cc2)c2cccnc21]

disclosed in Journal of Cystic Fibrosis (2018), 17(5), 595-606, and:

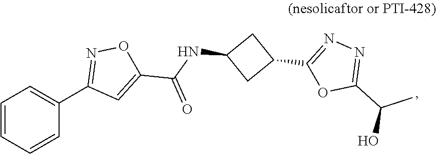 [SMILES: C[C@@H](O)c1nnc([C@H]2C[C@H](NC(=O)c3cc(-c4ccccc4)no3)C2)o1]

disclosed in WO 2016/105485. In one embodiment, the additional CFTR modulating agent is ASP-11. In one embodiment, the additional CFTR modulating agent comprises PTI-428.

Another aspect of the invention provides methods of treating the CFTR-mediated disease cystic fibrosis comprising administering to a patient in need thereof at least one compound chosen from compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, and optionally further administering one or more additional CFTR modulating agents selected from:

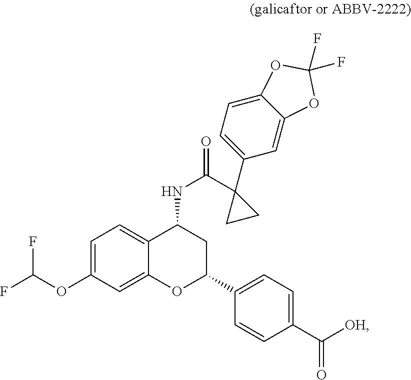 [SMILES: O=C(O)c1ccc([C@H]2C[C@@H](NC(=O)C3(c4ccc5c(c4)OC(F)(F)O5)CC3)c3ccc(OC(F)F)cc3O2)cc1]

disclosed in United States Patent Application Publication No. 2016-0120841;

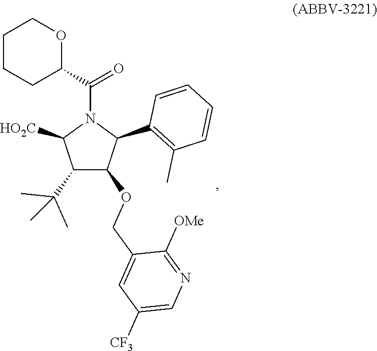 [SMILES: COc1ncc(C(F)(F)F)cc1CO[C@H]1[C@H](C(C)(C)C)[C@@H](C(=O)O)N(C(=O)[C@@H]2CCCCO2)[C@H]1c1ccccc1C]

disclosed in WO 2018/065921;

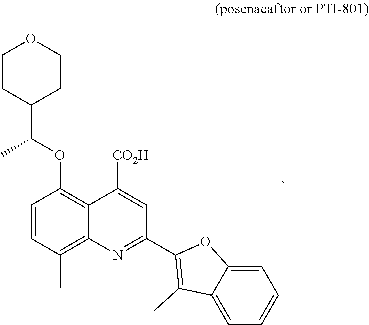 [SMILES: Cc1c(-c2cc(C(=O)O)c3c(O[C@H](C)C4CCOCC4)ccc(C)c3n2)oc2ccccc12]

disclosed in WO 2017/062581; ABBV-2851, disclosed in WO 2017/009804; GLPG2737, disclosed in United States Patent Application Publication No. 2017-0101405; ABBV-3748; ABBV-3903; and ABBV-119.

### Definitions

“Compound II,” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

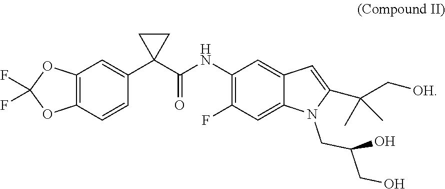 [SMILES: CC(C)(CO)c1cc2cc(NC(=O)C3(c4ccc5c(c4)OC(F)(F)O5)CC3)c(F)cc2n1C[C@@H](O)CO]

Compound II may be in the form of a pharmaceutically acceptable salt. Compound II and methods of making and using Compound II are disclosed in WO 2010/053471, WO 2011/119984, WO 2011/133751, WO 2011/133951, and WO 2015/160787, each incorporated herein by reference.

“Compound III” as used throughout this disclosure refers to N-(5-hydroxy-2,4-di-tert-butyl-phenyl)-4-oxo-1H-quinoline-3-carboxamide (also known as N-[2,4-bis(1,1-dimethylethyl)-5-hydroxyphenyl]-1,4-dihydro-4-oxoquinoline-3-carboxamide), which is depicted by the structure:

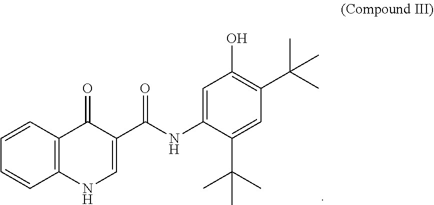 [SMILES: CC(C)(C)c1cc(C(C)(C)C)c(NC(=O)c2c[nH]c3ccccc3c2=O)cc1O]

Compound III may also be in the form of a pharmaceutically acceptable salt. Compound III and methods of making and using Compound III are disclosed in WO 2006/002421, WO 2007/079139, WO 2010/108162, and WO 2010/019239, each incorporated herein by reference.

In some embodiments, a deuterated derivative of Compound III (Compound III-d) is employed in the compositions and methods disclosed herein. A chemical name for Compound III-d is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

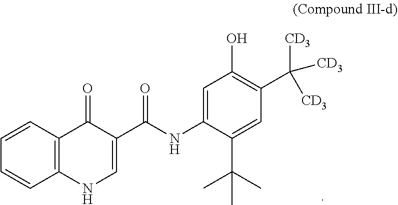 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Compound III-d may be in the form of a pharmaceutically acceptable salt. Compound III-d and methods of making and using Compound III-d are disclosed in WO 2012/158885, WO 2014/078842, WO 2019/109021, and U.S. Pat. No. 8,865,902, incorporated herein by reference.

“Compound IV” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

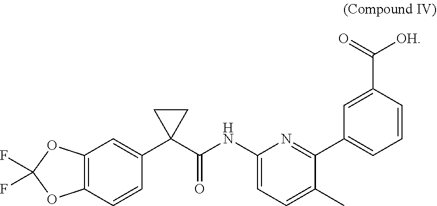 [SMILES: Cc1ccc(NC(=O)C2(c3ccc4c(c3)OC(F)(F)O4)CC2)nc1-c1cccc(C(=O)O)c1]

Compound IV may be in the form of a pharmaceutically acceptable salt. Compound IV and methods of making and using Compound IV are disclosed in WO 2007/056341, WO 2009/073757, and WO 2009/076142, incorporated herein by reference.

“Compound V” as used herein, refers to N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide, which is depicted by the chemical structure:

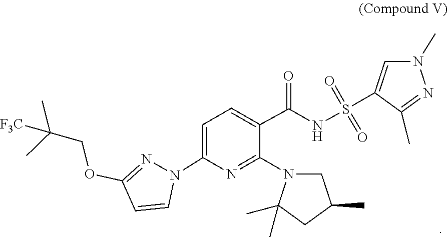 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

Compound V may be in the form of a pharmaceutically acceptable salt. Compound V and methods of making and using Compound V are disclosed in WO 2018/107100 and WO 2019/113476, incorporated herein by reference.

“Compound VI” as used herein, refers to N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide, which is depicted by the chemical structure:

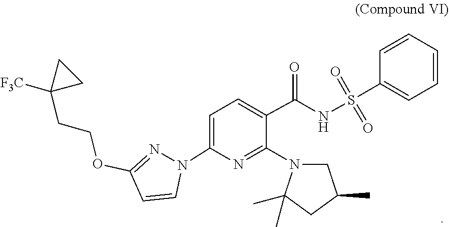 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

Compound VI may be in the form of a pharmaceutically acceptable salt. Compound VI and methods of making and using Compound VI are disclosed in WO 2018/064632 and WO 2019/113476, incorporated herein by reference.

“Compound VII” as used herein, refers to (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, which is depicted by the chemical structure:

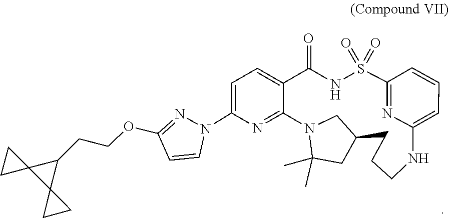 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

Compound VII may be in the form of a pharmaceutically acceptable salt. Compound VII and methods of making and using Compound VII are disclosed in WO 2019/161078, WO 2020/102346, and PCT Application No. PCT/US2020/046116, incorporated herein by reference.

“Compound VIII” as used herein, refers to (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione, which is depicted by the chemical structure:

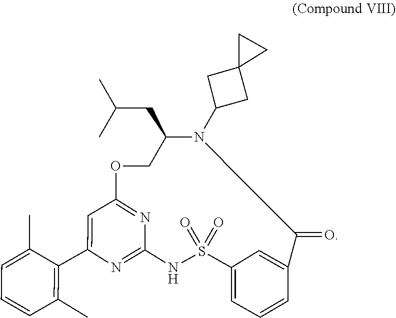 [SMILES: Cc1cccc(C)c1-c1cc2nc(n1)NS(=O)(=O)c1cccc(c1)C(=O)N(C1CC3(CC3)C1)[C@H](CC(C)C)CO2]

Compound VIII may be in the form of a pharmaceutically acceptable salt. Compound VIII and methods of making and using Compound VIII are disclosed in WO 2020/206080, incorporated herein by reference.

“Compound IX” as used herein, refers to N-(benzenesulfonyl)-6-(3-fluoro-5-isobutoxy-phenyl)-2-[(4S)-2,2,4-trimethylpyrrolidin-1-yl]pyridine-3-carboxamide, which is depicted by the chemical structure:

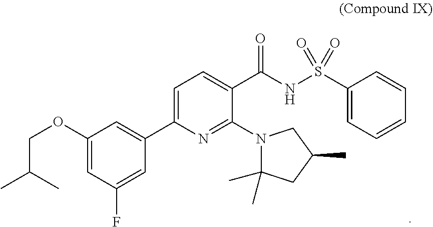 [SMILES: CC(C)COc1cc(F)cc(-c2ccc(C(=O)NS(=O)(=O)c3ccccc3)c(N3C[C@@H](C)CC3(C)C)n2)c1]

Compound IX may be in the form of a pharmaceutically acceptable salt. Compound IX and methods of making and using Compound IX are disclosed in WO 2016/057572, incorporated herein by reference.

“Compound X” as used herein, refers to N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, which is depicted by the chemical structure:

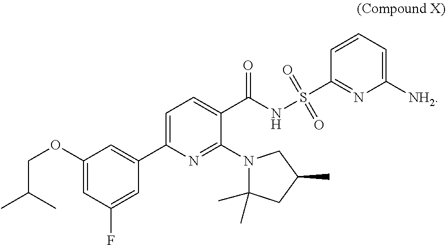 [SMILES: CC(C)COc1cc(F)cc(-c2ccc(C(=O)NS(=O)(=O)c3cccc(N)n3)c(N3C[C@@H](C)CC3(C)C)n2)c1]

Compound X may be in the form of a pharmaceutically acceptable salt. Compound X and methods of making and using Compound X are disclosed in WO 2016/057572, incorporated herein by reference.

As used herein, the term “alkyl” refers to a saturated, branched or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms). Alkyl groups may be substituted or unsubstituted.

As used herein, the term “pi bond” refers to a covalent bond formed by the p orbitals of adjacent atoms. Pi bonds exist where there is a multiple bond, i.e., a double or triple bond, between two atoms. For example, a carbon-carbon double bond consists of one pi bond, and a carbon-carbon triple bond consists of two pi bonds.

As used herein, the term “haloalkyl group” refers to an alkyl group substituted with one or more halogen atoms.

As used herein, the term “fluoroalkyl” refers to an alkyl group substituted with one or more fluorine atoms. In some embodiments, a fluoroalkyl group is substituted by 1-6 fluorine atoms. In some embodiments, a fluoroalkyl group is perfluorinated.

The term “alkoxy” as used herein refers to an alkyl or cycloalkyl covalently bonded to an oxygen atom. Alkoxy groups may be substituted or unsubstituted.

As used herein, the term “haloalkoxy group” refers to an alkoxy group substituted with one or more halogen atoms.

As used herein, the term “fluoroalkoxy” refers to an alkoxy group substituted with one or more fluorine atoms. In some embodiments, a fluoroalkoxy group is substituted by 1-6 fluorine atoms. In some embodiments, a fluoroalkoxy group is perfluorinated.

As used herein, “cycloalkyl” refers to a cyclic, bicyclic, tricyclic, or polycyclic non-aromatic hydrocarbon groups having 3 to 12 carbons (such as, for example 3-10 carbons). “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, and dispiro[2.0.2.1]heptane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The terms “heteroaryl ring” and “heteroaryl” as used herein refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline.

As used herein, the terms “heterocyclyl ring” and “heterocyclyl” refer to a non-aromatic hydrocarbon containing 3 to 12 atoms in a ring (such as, for example 3-10 atoms) comprising at least one ring atom that is a heteroatom, such as O, N, S, or Si and may include one or more unsaturated bonds. “Heterocyclyl” rings encompass monocyclic, bicyclic, tricyclic, polycyclic, bridged, fused, and spiro rings, including mono spiro and dispiro rings.

“Substituted” indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” means the same chemical structure, with one or more hydrogen atoms replaced by a deuterium atom.

As used herein, “CFTR” means cystic fibrosis transmembrane conductance regulator.

As used herein, the terms “CFTR modulator,” “CFTR-modulating compound,” and “CFTR-modulating agent” interchangeably refer to a compound that directly or indirectly increases the activity of CFTR. The increase in activity resulting from a CFTR modulator includes but is not limited to compounds that correct, potentiate, stabilize and/or amplify CFTR.

As used herein, the term “CFTR corrector” refers to a compound that facilitates the processing and trafficking of CFTR to increase the amount of CFTR at the cell surface. Compound II, Compound IV, Compound V, Compound VI, Compound VII, Compound VIII, Compound IX, and Compound X disclosed herein are CFTR correctors.

As used herein, the term “CFTR potentiator” refers to a compound that increases the channel activity of CFTR protein located at the cell surface, resulting in enhanced ion transport. The novel compounds disclosed herein are CFTR potentiators. Compound III and Compound III-d disclosed herein are CFTR potentiators.

As used herein, the terms “CFTR potentiator enhancer”, CFTR potentiation enhancer”, and “CFTR co-potentiator” are used interchangeably and refer to a compound that enhances CFTR potentiation.

As used herein, the term “active pharmaceutical ingredient” (“API”) or “therapeutic agent” refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately,” when used in connection with doses, amounts, or weight percentages of ingredients of a composition or a dosage form, include the value of a specified dose, amount, or weight percentage or a range of the dose, amount, or weight percentage that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within 15%, 10%, 5%, 4%, 3%, 2%, 1%, or 0.5% of a given value or range.

As used herein, the term “room temperature” or “ambient temperature” means 15° C. to 30° C.

It will be appreciated that certain compounds of this invention may exist as separate stereoisomers or enantiomers and/or mixtures of those stereoisomers or enantiomers. As used in the chemical structures disclosed herein, a “wedge” ( [symbol] ) or “hash” ( [symbol] ) bond to a stereogenic atom indicates a chiral center of known absolute stereochemistry (i.e. one stereoisomer). As used in the chemical structures disclosed herein, a “wavy” bond ( [symbol] ) to a stereogenic atom indicates a chiral center of unknown absolute stereochemistry (i.e. one stereoisomer). As used in the chemical structures disclosed herein, a “wavy” bond ( [symbol] ) to a double-bonded carbon indicates a mixture of E/Z isomers. As used in the chemical structures disclosed herein, a [symbol] (“straight”) bond to a stereogenic atom indicates where there is a mixture (e.g., a racemate or enrichment). As used herein, two [symbol] (“straight”) bonds to a double-bonded carbon indicates that the double bond possesses the E/Z stereochemistry as drawn. As used in the chemical structures disclosed herein, a [symbol] (i.e., a “wavy” line perpendicular to a “straight” bond to group “A”) indicates that group “A” is a substituent whose point of attachment is at the end of the bond that terminates at the “wavy” line.

Certain compounds disclosed herein may exist as tautomers and both tautomeric forms are intended, even though only a single tautomeric structure is depicted. For example, a description of Compound A is understood to include its tautomer Compound B and vice versa, as well as mixtures thereof:

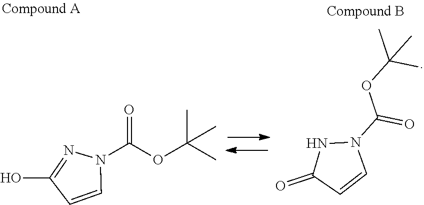

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure wherein the salt is nontoxic. Pharmaceutically acceptable salts of the compounds of this disclosure include those derived from suitable inorganic and organic acids and bases. A “free base” form of a compound, for example, does not contain an ionically bonded salt.

The phrase “deuterated derivative or pharmaceutically acceptable salt thereof” is used interchangeably with “deuterated derivative thereof or pharmaceutically acceptable salt of any of the forgoing” in reference to one or more compounds or formulae of the invention. The phrase “pharmaceutically acceptable salt and deuterated derivative thereof” is used interchangeably with “pharmaceutically acceptable salt thereof and deuterated derivative of any of the forgoing” in reference to one or more compounds or formulae of the invention.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | Triethiodide |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

### DETAILED DESCRIPTION OF EMBODIMENTS

In addition to compounds of Formula I, pharmaceutically acceptable salts thereof, and deuterated derivatives of those compounds and salts, the invention provides compounds of Formulae I′, Ia, Ib, Ia′, Ib′, IIa, IIb, IIc, IId, IIe, and IIf, Compounds 1-271, deuterated derivatives thereof, and pharmaceutically acceptable salts of any of the foregoing.

For example, in some embodiments, the compound of Formula I is selected from compounds of any one of Formulae Ta and Tb:

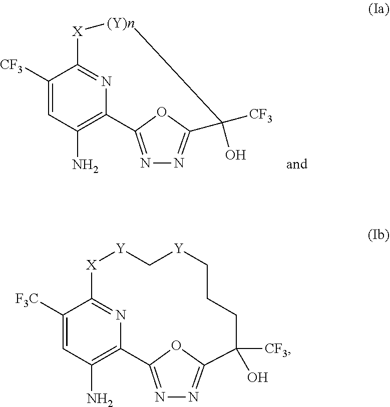

and deuterated derivatives and pharmaceutically acceptable salts thereof, wherein all variables are as defined for Formula I.

For example, in some embodiments, the compound of Formula I is selected from compounds of any one of Formulae Ia′ and Ib′:

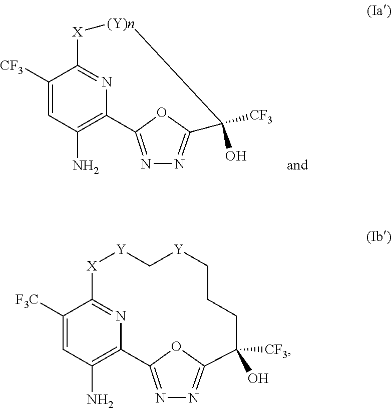

and deuterated derivatives and pharmaceutically acceptable salts thereof, wherein all variables are as defined for Formula I.

For example, in some embodiments, the compound of Formula I is selected from compounds of any one of Formulae IIa, IIb, IIc, IId, IIe, and IIf:

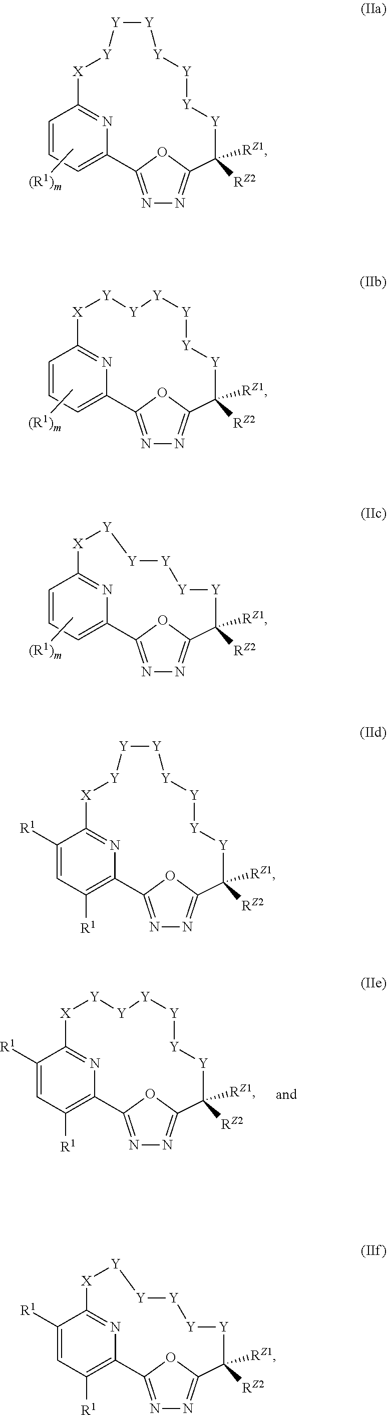

and deuterated derivatives and pharmaceutically acceptable salts thereof, wherein all variables are as defined for Formula I.

Also disclosed herein are compounds having a formula chosen from any one of the formulae depicted in Table 7, deuterated derivatives thereof, and pharmaceutically acceptable salts of any of those compounds and deuterated derivatives.

### Methods of Treatment

Any of the novel compounds disclosed herein, such as for example, compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the invention provides methods of treating, lessening the severity of, or symptomatically treating cystic fibrosis in a patient comprising administering to the patient an effective amount of any of the novel compounds disclosed herein, such as for example, compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, alone or in combination with another active ingredient, such as another CFTR modulating agent. In some embodiments, the patient has an F508del/minimal function (MF) genotype, F508del/F508del genotype (homozygous for the F508del mutation), F508del/gating genotype, or F508del/residual function (RF) genotype. In some embodiments the patient is heterozygous and has one F508del mutation. In some embodiments, the patient is homozygous for the F508del mutation. In some embodiments the patient is homozygous for the N1303K mutation. In some embodiments, the patient has a G551D mutation.

In some embodiments, 1 mg to 1000 mg of a compound disclosed herein, a deuterated derivative thereof or a pharmaceutically acceptable salt of the compound or deuterated derivative are administered daily.

In some embodiments, the patient is heterozygous and has an F508del mutation on one allele and a mutation on the other allele selected from Table 2:

TABLE 2
CFTR Mutations
Mutation

| | | | | |
|---|---|---|---|---|
| Q2X | L218X | Q525X | R792X | E1104X |
| S4X | Q220X | G542X | E822X | W1145X |
| W19X | Y275X | G550X | W882X | R1158X |
| G27X | C276X | Q552X | W846X | R1162X |
| Q39X | Q290X | R553X | Y849X | S1196X |
| W57X | G330X | E585X | R851X | W1204X |
| E60X | W401X | G673X | Q890X | L1254X |
| R75X | Q414X | Q685X | S912X | S1255X |
| L88X | S434X | R709X | Y913X | W1282X |
| E92X | S466X | K710X | Q1042X | Q1313X |
| Q98X | S489X | Q715X | W1089X | Q1330X |
| Y122X | Q493X | L732X | Y1092X | E1371X |
| E193X | W496X | R764X | W1098X | Q1382X |
| W216X | C524X | R785X | R1102X | Q1411X |
| 185 + 1G→T | 711 + 5G→A | 1717 − 8G→A | 2622 + 1G→A | 3121 − 1G→A |
| 296 + 1G→A | 712 − 1G→T | 1717 − 1G→A | 2790 − 1G→C | 3500 − 2A→G |
| 296 + 1G→T | 1248 + 1G→A | 1811 + 1G→C | 3040G→C | 3600 + 2insT |
| 405 + 1G→A | 1249 − 1G→A | 1811 + 1.6kbA→G | (G970R) | 3850 − 1G→A |
| 405 + 3A→C | 1341 + 1G→A | 1811 + 1643G→T | 3120G→A | 4005 + 1G→A |
| 406 − 1G→A | 1525 − 2A→G | 1812 − 1G→A | 3120 + 1G→A | 4374 + 1G→T |
| 621 + 1G→T | 1525 − 1G→A | 1898 + 1G→A | 3121 − 2A→G | |
| 711 + 1G→T | | 1898 + 1G→C | | |
| 182delT | 1078delT | 1677delTA | 2711delT | 3737delA |
| 306insA | 1119delA | 1782delA | 2732insA | 3791delC |
| 306delTAGA | 1138insG | 1824delA | 2869insG | 3821delT |
| 365-366insT | 1154insTC | 1833delT | 2896insAG | 3876delA |
| 394delTT | 1161delC | 2043delG | 2942insT | 3878delG |
| 442delA | 1213delT | 2143delT | 2957delT | 3905insT |
| 444delA | 1259insA | 2183AA→G | 3007delG | 4016insT |
| 457TAT→G | 1288insTA | 2184delA | 3028delA | 4021dupT |
| 541delC | 1343delG | 2184insA | 3171delC | 4022insT |
| 574delA | 1471delA | 2307insA | 3171insC | 4040delA |
| 663delT | 1497delGG | 2347delG | 3271delGG | 4279insA |
| 849delG | 1548delG | 2585delT | 3349insT | 4326delTC |
| 935delA | 1609del CA | 2594delGT | 3659delC | |
| CFTRdele1 | | CFTRdele16-17b | 1461ins4 | |
| CFTRdele2 | | CFTRdele17a, 17b | 1924del7 | |
| CFTRdele2, 3 | | CFTRdele17a-18 | 2055del9→A | |
| CFTRdele2-4 | | CFTRdele19 | 2105-2117del13insAGAAA | |
| CFTRdele3-10, 14b-16 | | CFTRdele19-21 | 2372del8 | |
| CFTRdele4-7 | | CFTRdele21 | 2721del11 | |
| CFTRdele4-11 | | CFTRdele22-24 | 2991del32 | |
| CFTR50kbdel | | CFTRdele22, 23 | 3667ins4 | |
| CFTRdup6b-10 | | 124del23bp | 4010del4 | |
| CFTRdele11 | | 602del14 | 4209TGTT→AA | |
| CFTRdele13, 14a | | 852del22 | | |
| CFTRdele14b-17b | | 991del5 | | |
| A46D | V520F | Y569D | N1303K | |
| G85E | A559T | L1065P | | |
| R347P | R560T | R1066C | | |
| L467P | R560S | L1077P | | |
| I507del | A561E | M1101K | | |

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²P, ³⁵S, ¹⁸F, and ³⁶Cl, respectively.

The isotope-labelled compounds and salts can be used in a number of beneficial ways. They can be suitable for medicaments and/or various types of assays, such as substrate tissue distribution assays. For example, tritium (³H)— and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “²H” or “D.”

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

The deuterium (²H)-labelled compounds and salts can modulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, which is incorporated herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR-mediated diseases using any of the novel compounds disclosed herein, such as for example, compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of the foregoing, in combination with at least one additional active pharmaceutical ingredient.

Thus, in some embodiments, the invention provides methods of treating, lessening the severity of, or symptomatically treating cystic fibrosis in a patient comprising administering to the patient an effective amount of any of the novel compounds disclosed herein, such as for example, compounds of Formulae I, I′, Ia, Ib, Ia′, Ib′, IIa, IIb, IIc, IId, IIe, and IIf, Compounds 1-271, deuterated derivatives thereof, and pharmaceutically acceptable salts of any of those compounds and deuterated derivatives, alone or in combination with at least one additional active pharmaceutical ingredient, such as, e.g., a CFTR modulating agent.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levofloxacin, including aerosolized formulations thereof, and combinations of two antibiotics, e.g., fosfomycin and tobramycin.

In some embodiments, the additional agent is a mucolyte. Exemplary mucolytes useful herein includes Pulmozyme®.

In some embodiments, the additional agent is a bronchodilator. Exemplary bronchodilators include albuterol, metaproterenol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simvastatin.

In some embodiments, the additional agent is a nutritional agent. Exemplary nutritional agents include pancrelipase (pancreating enzyme replacement), including Pancrease®, Pancreacarb®, Ultrase®, or Creon®, Liprotomase® (formerly Trizytek®), Aquadeks®, or glutathione inhalation. In one embodiment, the additional nutritional agent is pancrelipase.

In some embodiments, at least one additional active pharmaceutical ingredient is selected from CFTR modulating agents. In some embodiments, the CFTR modulating agent is a CFTR corrector. In some embodiments, the CFTR modulating agent is a CFTR potentiator enhancer/co-potentiator (for example, ASP-11). In some embodiments, the at least one additional active pharmaceutical ingredient is a CFTR amplifier. In some embodiments, the at least one additional active pharmaceutical ingredient is a CFTR readthrough agent. In some embodiments, the at least one additional active pharmaceutical ingredient is a CFTR nucleic acid therapy.

In some embodiments, the at least one additional active pharmaceutical ingredient is a ENaC inhibitor. In some embodiments, the at least one additional active pharmaceutical ingredient is a TMEM16A modulator. In some embodiments, the at least one additional active pharmaceutical ingredient is a GPR39 agonist.

In some embodiments, the at least one additional active pharmaceutical ingredient is chosen from (a) Compound II and deuterated derivatives and pharmaceutically acceptable salts thereof; (b) Compound IV and deuterated derivatives and pharmaceutically acceptable salts thereof; (c) Compound V and deuterated derivatives and pharmaceutically acceptable salts thereof, (d) Compound VI and deuterated derivatives and pharmaceutically acceptable salts thereof; (e) Compound VII and deuterated derivatives and pharmaceutically acceptable salts thereof; and (f) Compound VIII and deuterated derivatives and pharmaceutically acceptable salts thereof. Thus, in some embodiments, the combination therapies provided herein comprise a compound selected from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; and at least one compound chosen from Compound II, Compound IV, Compound V, Compound VI, Compound VII, Compound VIII, Compound IX, Compound X, and deuterated derivatives and pharmaceutically acceptable salts thereof. In some embodiments, the combination therapies provided herein comprise (a) at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; (b) at least one compound chosen from Compound II, Compound IV, and pharmaceutically acceptable salts and deuterated derivatives thereof, and (c) at least one compound chosen from Compound V, Compound VI, Compound VII, Compound VIII, Compound IX, Compound X, and deuterated derivatives and pharmaceutically acceptable salts thereof. In some embodiments, the combination therapies provided herein comprise (a) at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; (b) at least one compound selected from Compound II and pharmaceutically acceptable salts and deuterated derivatives thereof; and (c) at least one compound chosen from Compound VII and deuterated derivatives and pharmaceutically acceptable salts thereof.

In some embodiments, the combination therapies provided herein comprise (a) a compound selected from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; (b) at least one compound chosen from Compound II, Compound IV, Compound V, Compound VI, Compound VII, Compound VIII, Compound IX, Compound X, and deuterated derivatives and pharmaceutically acceptable salts thereof; and (c) at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P. W. et al. J. Cyst. Fibros. 2018, 17 (5), 595-606; Pedemonte, N. et al. Sci. Adv. 2020, 6 (8), eaay9669; Phuan, P. W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N.J, Solovay, C. F., Pharm. Pat. Anal. 2017, 6 (4), 179-188; Kunzelmann, K. et al., Front. Pharmacol. 2019, 10, 3; or Son, J. H. et al., Eur. J. of Med. Chem. 2020, 112888.

In some embodiments, the combination therapies provided herein comprise (a) a compound selected from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; (b) at least one compound chosen from Compound II, Compound IV, Compound V, Compound VI, Compound VII, Compound VIII, Compound IX, Compound X, and deuterated derivatives and pharmaceutically acceptable salts thereof; and (c) at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039.

In some embodiments, the combination therapies provided herein comprise (a) a compound selected from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; and (b) at least two compounds chosen from compounds disclosed in WO 2019/195739, WO 2019/200246, WO 2021/030555, WO 2021/030556, WO 2010/053471, WO 2011/119984, WO 2011/133751, WO 2011/133951, WO 2015/160787, WO 2007/056341, WO 2009/073757, WO 2009/076142, WO 2018/107100, WO 2019/113476, WO 2018/064632, WO 2019/152940, WO 2016/057572, WO 2021/030554, WO 2020/206080, WO 2016/105485, United States Patent Application Publication No. 2016-0120841, United States Patent Application Publication No. 2017-0101405, WO 2017/009804, WO 2018/065921, WO 2017/062581; Phuan, P. W. et al. J. Cyst. Fibros. 2018, 17 (5), 595-606; Pedemonte, N. et al. Sci. Adv. 2020, 6 (8), eaay9669; Phuan, P. W. et al. Sci. Rep. 2019, 9 (1), 17640; Bose, S. et al. J. Cyst. Fibros. 2020, 19 Suppl 1, S25-S32; Crawford, D. K. J. Pharmacol. Exp. Ther. 2020, 374 (2), 264-272; Brasell, E. J. et al. PLoS One 2019, 14 (12), e0223954; Smith, N.J, Solovay, C. F., Pharm. Pat. Anal. 2017, 6 (4), 179-188; Kunzelmann, K. et al., Front. Pharmacol. 2019, 10, 3; or Son, J. H. et al., Eur. J. of Med. Chem. 2020, 112888.

In some embodiments, the combination therapies provided herein comprise (a) a compound selected from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives; and (b) at least two compounds chosen from Compound II, Compound IV, Compound V, Compound VI, Compound VII, Compound VIII, Compound IX, Compound X, PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039, and deuterated derivatives and pharmaceutically acceptable salts thereof.

In some embodiments, at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives, is administered in combination with at least one compound chosen from Compound II and deuterated derivatives and pharmaceutically acceptable salts thereof. In some embodiments, at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of those compounds and deuterated derivatives, is administered in combination with at least one compound chosen from Compound IV and deuterated derivatives and pharmaceutically acceptable salts thereof. In some embodiments, at least one compound chosen from compounds Formulae I, I′, Ia, Ib, Ia′, Ib′, IIa, IIb, IIc, IId, IIe, and IIf, Compounds 1-271, deuterated derivatives of those compounds, and pharmaceutically acceptable salts of those compounds and deuterated derivatives, is administered in combination with at least one compound chosen from Compound V and deuterated derivatives and pharmaceutically acceptable slats thereof. In some embodiments, at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of those compounds and deuterated derivatives, is administered in combination with at least one compound chosen from Compound VI and deuterated derivatives and pharmaceutically acceptable salts thereof. In some embodiments, at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives, is administered in combination with at least one compound chosen from Compound VII and deuterated derivatives and pharmaceutically acceptable salts thereof. In some embodiments, at least one compound chosen from compounds of Formulae I, I′, Ia, Ib, Ia′, Ib′, IIa, IIb, IIc, IId, IIe, and IIf, Compounds 1-271, deuterated derivatives of those compounds, and pharmaceutically acceptable salts of any of the foregoing compounds and deuterated derivatives, is administered in combination with at least one compound chosen from Compound VIII and deuterated derivatives and pharmaceutically acceptable salts thereof.

Each of the compounds of Formulae I, I′, Ia, Ib, Ia′, Ib′, IIa, IIb, IIc, IId, IIe, and IIf, Compounds 1-271, Compound II, Compound IV, Compound V, Compound VI, Compound VII, Compound VIII, Compound IX, Compound X, and their deuterated derivatives and pharmaceutically acceptable salts thereof, independently can be administered once daily, twice daily, or three times daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof is administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof is administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound II and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound II and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound IV and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound IV and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound V and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound V and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VI and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VI and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VII and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VII and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VIII and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least one compound chosen from Compound VIII and pharmaceutically acceptable salts thereof are administered twice daily.

In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof; at least one compound chosen from Compound II, Compound IV, and pharmaceutically acceptable salts thereof, and at least one compound chosen from Compound V, Compound VI, Compound VII, Compound VIII, Compound IX, Compound X, and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, at least one compound chosen from Compound II, Compound IV, and pharmaceutically acceptable salts thereof; and at least one compound chosen from Compound V, Compound VI, Compound VII, Compound VIII, Compound IX, Compound X, and pharmaceutically acceptable salts thereof are administered twice daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof; at least one compound chosen from Compound II and pharmaceutically acceptable salts thereof, and at least one compound chosen from Compound VII and pharmaceutically acceptable salts thereof are administered once daily. In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, at least one compound chosen from Compound II and pharmaceutically acceptable salts thereof, and at least one compound chosen from Compound VII and pharmaceutically acceptable salts thereof are administered twice daily.

Compounds of Formulae I, I′, Ia, Ib, Ia′, Ib′, IIa, IIb, IIc, IId, IIe, and IIf, Compounds 1-271, Compound II, Compound IV, Compound V, Compound VI, Compound VII, Compound VIII, Compound IX, Compound X, and their deuterated derivatives and pharmaceutically acceptable salts thereof can be administered in a single pharmaceutical composition or separate pharmaceutical compositions. Such pharmaceutical compositions can be administered once daily or multiple times daily, such as twice daily. As used herein, the phrase that a given amount of API (e.g., Compound II, Compound VII, or pharmaceutically acceptable salts thereof) is administered once or twice daily or per day means that said given amount is administered per dosing, which may occur once or twice daily.

In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof is administered in a first pharmaceutical composition; and at least one compound chosen from Compound VII and pharmaceutically acceptable salts thereof is administered in a second pharmaceutical composition.

In some embodiments, at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof is administered in a first pharmaceutical composition; at least one compound chosen from Compound II and pharmaceutically acceptable salts thereof is administered in a second pharmaceutical composition; and at least one compound chosen from Compound VII and pharmaceutically acceptable salts thereof is administered in a third pharmaceutical composition.

Any suitable pharmaceutical compositions known in the art can be used for compounds of Formulae I, I′, Ia, Ib, Ia′, Ib′, IIa, IIb, IIc, IId, IIe, and IIf, Compounds 1-271, Compound II, Compound IV, Compound V, Compound VI, Compound VII, Compound VIII, Compound IX, Compound X, and deuterated derivatives and pharmaceutically acceptable salts thereof. Some exemplary pharmaceutical compositions for Compound II and its pharmaceutically acceptable salts can be found in WO 2011/119984 and WO 2014/014841, incorporated herein by reference. Some exemplary pharmaceutical compositions for Compound III and its pharmaceutically acceptable salts can be found in WO 2007/134279, WO 2010/019239, WO 2011/019413, WO 2012/027731, and WO 2013/130669, and some exemplary pharmaceutical compositions for Compound III-d and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for Compound IV and its pharmaceutically acceptable salts can be found in WO 2010/037066, WO 2011/127421, and WO 2014/071122, incorporated herein by reference. Some exemplary pharmaceutical compositions for Compound V and its pharmaceutically acceptable salts can be found in WO 2019/152940, incorporated herein by reference. Some exemplary pharmaceutical compositions for Compound VI and its pharmaceutically acceptable salts can be found in WO 2019/079760, incorporated herein by reference.

### Pharmaceutical Compositions

Another aspect of the invention provides a pharmaceutical composition comprising at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, and at least one pharmaceutically acceptable carrier.

In some embodiments, the invention provides pharmaceutical compositions comprising at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the at least one additional active pharmaceutical ingredient is a compound that enhances CFTR potentiation, i.e., a CFTR potentiator enhancer/co-potentiator. In some embodiments, the at least one additional active pharmaceutical ingredient is a CFTR amplifier. In some embodiments, the at least one additional active pharmaceutical ingredient is a CFTR readthrough agent. In some embodiments, the at least one additional active pharmaceutical ingredient is a CFTR nucleic acid therapy. In some embodiments, the at least one additional active pharmaceutical ingredient is a ENaC inhibitor. In some embodiments, the at least one additional active pharmaceutical ingredient is a TMEM16A modulator. In some embodiments, the at least one additional active pharmaceutical ingredient is a GPR39 agonist. In some embodiments, the pharmaceutical composition comprises at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least two additional active pharmaceutical ingredients, each of which is a CFTR corrector. In some embodiments, the pharmaceutical composition comprises at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof and at least two additional active pharmaceutical ingredients, one of which is a CFTR corrector and one of which is a CFTR potentiator enhancer.

In some embodiments, the invention provides a pharmaceutical composition comprising (a) at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least one compound chosen from Compound II, Compound IV, Compound V, Compound VI, Compound VII, Compound VIII, Compound IX, Compound X, and pharmaceutically acceptable salts thereof, and (c) at least one pharmaceutically acceptable carrier.

In some embodiments, the invention provides a pharmaceutical composition comprising (a) at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least one compound chosen from Compound II and pharmaceutically acceptable salts thereof, and (c) at least one pharmaceutically acceptable carrier.

In some embodiments, the invention provides a pharmaceutical composition comprising (a) at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least one compound chosen from Compound VII and pharmaceutically acceptable salts thereof, and (c) at least one pharmaceutically acceptable carrier.

In some embodiments, the disclosure provides a pharmaceutical composition comprising (a) at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least one compound chosen from Compound II, Compound IV, and pharmaceutically acceptable salts thereof, (c) at least one compound chosen from Compound V, Compound VI, Compound VII, Compound VIII, Compound IX, Compound X, and pharmaceutically acceptable salts thereof, and (d) at least one pharmaceutically acceptable carrier.

In some embodiments, the disclosure provides a pharmaceutical composition comprising (a) at least one compound chosen from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least one compound chosen from Compound II and pharmaceutically acceptable salts thereof, (c) at least one compound chosen from Compound VII and pharmaceutically acceptable salts thereof, and (d) at least one pharmaceutically acceptable carrier.

In some embodiments, the pharmaceutical compositions provided herein comprise (a) a compound selected from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof; (b) at least one compound chosen from Compound II, Compound IV, Compound V, Compound VI, Compound VII, Compound VIII, Compound IX, Compound X, and pharmaceutically acceptable salts and deuterated derivatives thereof, (c) at least one compound chosen from compounds disclosed in WO 2016/105485, United States Patent Application Publication No. 2016-0120841, United States Patent Application Publication No. 2017-0101405, WO 2017/009804, WO 2018/065921, WO 2017/062581; Phuan, P. W. et al. J. Cyst. Fibros. 2018, 17 (5), 595-606; Pedemonte, N. et al. Sci. Adv. 2020, 6 (8), eaay9669; Phuan, P. W. et al. Sci. Rep. 2019, 9 (1), 17640; Bose, S. et al. J. Cyst. Fibros. 2020, 19 Suppl1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J. H. et al., Eur. J. of Med. Chem. 2020, 112888; and (d) at least one pharmaceutically acceptable carrier.

In some embodiments, the pharmaceutical compositions provided herein comprise (a) a compound selected from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof; (b) at least one compound chosen from Compound II, Compound IV, Compound V, Compound VI, Compound VII, Compound VIII, Compound IX, Compound X, and pharmaceutically acceptable salts and deuterated derivatives thereof, (c) at least one compound chosen from PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039; and (d) at least one pharmaceutically acceptable carrier.

In some embodiments, the pharmaceutical compositions provided herein comprise (a) a compound selected from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least two compounds chosen from compounds disclosed in WO 2019/195739, WO 2019/200246, WO 2021/030555, WO 2021/030556, WO 2010/053471, WO 2011/119984, WO 2011/133751, WO 2011/133951, WO 2015/160787, WO 2007/056341, WO 2009/073757, WO 2009/076142, WO 2018/107100, WO 2019/113476, WO 2018/064632, WO 2019/152940, WO 2016/057572, WO 2021/030554, WO 2020/206080, WO 2016/105485, United States Patent Application Publication No. 2016-0120841, United States Patent Application Publication No. 2017-0101405, WO 2017/009804, WO 2018/065921, WO 2017/062581; Phuan, P. W. et al. J. Cyst. Fibros. 2018, 17 (5), 595-606; Pedemonte, N. et al. Sci. Adv. 2020, 6 (8), eaay9669; Phuan, P. W. et al. Sci. Rep. 2019, 9 (1), 17640; Bose, S. et al. J. Cyst. Fibros. 2020, 19 Suppl1, S25-S32; Crawford, D. K. J. Pharmacol. Exp. Ther. 2020, 374 (2), 264-272; Brasell, E. J. et al. PLoS One 2019, 14 (12), e0223954; Smith, N. J, Solovay, C. F., Pharm. Pat. Anal. 2017, 6 (4), 179-188; Kunzelmann, K. et al., Front. Pharmacol. 2019, 10, 3; or Son, J. H. et al., Eur. J. of Med. Chem. 2020, 112888; and (c) at least one pharmaceutically acceptable carrier.

In some embodiments, the pharmaceutical compositions provided herein comprise (a) a compound selected from compounds of Formulae I, I′, Ia, Ib, Ia′, Ib′, IIa, IIb, IIc, IId, IIe, and IIf, Compounds 1-271, and deuterated derivatives and pharmaceutically acceptable salts thereof, (b) at least two compounds chosen from Compound II, Compound IV, Compound V, Compound VI, Compound VII, Compound VIII, Compound IX, Compound X, PTI-428, ASP-11, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, PTI-801, FDL-176, PTI-808, GLPG1837/ABBV-974, GLPG2451/ABBV-2451, QBW251 (Icenticaftor), GLPG3067/ABBV-3067 (Navocaftor), ABBV-191, ELX-02, MRT5005, Lunar-CF, RCT223, amiloride, ETD001, CF552, GS-9411, GS-5737, P-1037 (VX-371), P-1055 (VX-551), AZD5634, SPX-101, Ionis-ENaC-2.5 Rx, BI 1265162, ARO-ENaC1001, ETD002, and DS-1039, and pharmaceutically acceptable salts and deuterated derivatives thereof; and (c) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

The pharmaceutical compositions described herein are useful for treating cystic fibrosis and other CFTR-mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Non-Limiting Exemplary Embodiments

1. A compound selected from compounds of Formula I:

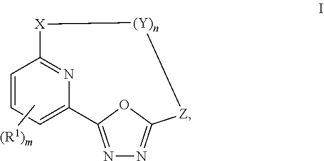

and deuterated derivatives and pharmaceutically acceptable salts thereof, wherein:

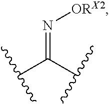

X is selected from —C(R^{X1})₂—, —CO—,

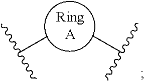

Si(R^{Z3})₂—, and;
Ring A is cyclic group selected from phenyl and 5- to 6-membered heteroaryl, wherein the cyclic group is optionally substituted with 1-3 groups independently selected from C₁-C₆ alkyl;
each R^{X1} is independently selected from H, C₁-C₆ alkyl (optionally substituted with 1-3 groups independently selected from oxo, —OR^{X2}, and —N(R^{X2})₂), C₃-C₈ cycloalkyl, halogen, cyano, —OR^{X2}, and C₁-C₆ fluoroalkyl;
each R^{X2} is independently selected from H and C₁-C₆ alkyl;
each Y is independently selected from —C(R^{Y})₂—, —O—, —CO—, —NR^{YN}—, and

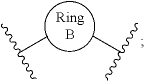

each R^{Y} is independently selected from hydrogen, hydroxy, halogen, C₁-C₆ alkyl (optionally substituted with 1-3 groups independently selected from hydroxy, C₁-C₆ alkoxy, and Q), C₃-C₈ cycloalkyl, C₆-C₁₀ aryl (optionally substituted with 1-3 groups independently selected from halogen), 5- to 10-membered heteroaryl, —OR^{Y1}, —CO₂R^{Y1}, —COR^{Y1}, —CON(R^{Y1})₂, and —N(R^{Y1})₂;
or two R^{Y}, one of which is on one atom and the second of which is on an adjacent atom, are taken together to form a pi bond;
each R^{Y1} is independently selected from hydrogen and C₁-C₆ alkyl, or two R^{Y1} bonded to the same nitrogen taken together form a 3- to 6-membered heterocyclyl;
each R^{YN} is independently selected from:
   H,
   C₁-C₆ alkyl, optionally substituted with 1-5 groups independently selected from:
      oxo,
      C₃-C₈ cycloalkyl (optionally substituted with 1-3 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₈ cycloalkyl (optionally substituted with 1-3 groups independently selected from halogen, C₁-C₆ alkyl, and C₁-C₆ fluoroalkyl), and
         phenyl (optionally substituted with 1-3 groups independently selected from C₁-C₆ alkyl, C₁-C₆ alkoxy, C₁-C₆ fluoroalkyl, C₁-C₆ fluoroalkoxy, and halogen),
      5- to 10-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN}1 is independently selected from H, C₁-C₄ alkyl (optionally substituted with oxo), and C₃-C₆ cycloalkyl;
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl,
   5- to 10-membered heteroaryl, and
   3- to 6-membered heterocyclyl (optionally substituted with 1-3 groups independently selected from C₁-C₆ alkyl);
each Q is independently selected from:
   C₁-C₆ alkyl optionally substituted with 1-3 groups independently selected from:
      halogen,
      oxo,
      C₆-C₁₀ aryl (optionally substituted with 1-3 groups independently selected from halogen and —OCF₃), and
      C₃-C₈ cycloalkyl,
   C₃-C₈ cycloalkyl optionally substituted with 1-3 groups independently selected from:
      halogen,
      CN,
      C₁-C₆ alkyl (optionally substituted with 1-3 groups independently selected from halogen, —NH₂, and —NHCOMe),
      C₁-C₆ alkoxy,
      C₆-C₁₀ aryl (optionally substituted with 1-3 groups independently selected from C₁-C₆ alkyl), and
      C₃-C₈ cycloalkyl,
   C₆-C₁₀ aryl optionally substituted with 1-3 groups independently selected from:
      halogen,
      CN,
      C₁-C₆ alkyl (optionally substituted with 1-3 groups independently selected from halogen and hydroxy),
      C₁-C₆ alkoxy optionally substituted with 1-4 groups independently selected from:
         halogen,
         C₃-C₈ cycloalkyl (optionally substituted with CF₃),
      C₃-C₅ cycloalkyl (optionally substituted with 1-3 groups independently selected from halogen, CF₃, OCF₃, and C₁-C₆ alkyl), and
      C₆-C₁₀ aryl,
   5- to 10-membered heteroaryl optionally substituted with 1-3 groups independently selected from:
      halogen,
      C₁-C₆ alkyl (optionally substituted with 1-3 groups independently selected from halogen),
      C₃-C₈ cycloalkyl (optionally substituted with 1-3 CF₃ groups), and
      3- to 10-membered heterocyclyl,
   3- to 10-membered heterocyclyl optionally substituted with 1-3 groups independently selected from:
      C₁-C₆ alkyl (optionally substituted with 1-3 groups independently selected from oxo and C₃-C₈ cycloalkyl), and
      oxo;
each R¹ is independently selected from halogen, C₁-C₆ fluoroalkyl, C₁-C₆ alkyl (optionally substituted with a group selected from hydroxy, C₆-C₁₀ aryl, and 5- to 6-membered heteroaryl), —OR², —N(R²)₂, —CO₂R², —CO—N(R²)₂, —CN, C₃-C₈ cycloalkyl, C₆-C₁₀ aryl, 5- to 6-membered heteroaryl (optionally substituted with 1-3 groups independently selected from C₁-C₆ alkyl), 3- to 6-membered heterocyclyl, —B(OR²)₂, —SO₂R², —SR², —SOR², —PO(OR²)₂, and —PO(R²)₂;
each R² is independently selected from hydrogen, C₁-C₆ alkyl (optionally substituted with 1-6 groups independently selected from halogen), C₁-C₆ fluoroalkyl, and C₆-C₁₀ aryl (optionally substituted with 1-3 groups independently selected from C₁-C₆ fluoroalkyl and C₁-C₆ fluoroalkoxy);
Z is selected from

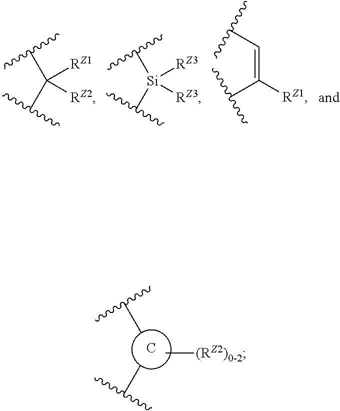

wherein Ring C is selected from C₆-C₁₀ aryl and 5- to 10-membered heteroaryl;
R^{Z1} is selected from hydrogen, —CN, C₁-C₆ alkyl (optionally substituted with 1-3 hydroxy), C₁-C₆ fluoroalkyl, 3- to 6-membered heterocyclyl, C₃-C₆ cycloalkyl, C₆-C₁₀ aryl, and 5- to 6-membered heteroaryl;
R^{Z2} is selected from hydrogen, halogen, hydroxy, NH₂, NH(CO)(C₁-C₆ alkyl), and C₁-C₆ alkoxy (optionally substituted with 1-3 groups independently selected from C₃-C₁₀ cycloalkyl),
or R^{Z1} and R^{Z2} taken together form a group selected from oxo and ═N—OH;
each R^{Z3} is independently selected from hydroxy, C₁-C₆ alkoxy, C₁-C₆ alkyl, and C₆-C₁₀ aryl; or two R^{Z3} are taken together to form a 3- to 6-membered heterocyclyl;
n is selected from 4, 5, 6, 7, and 8; and
m is selected from 0, 1, 2, and 3.
   1a. A compound selected from compounds of Formula I′:

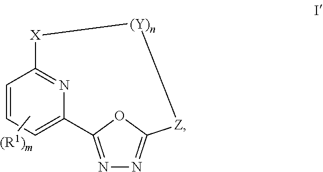

and deuterated derivatives and pharmaceutically acceptable salts thereof, wherein:
X is selected from —C(R^{X1})₂—, —CO—,

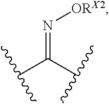

—Si(R^{Z3})₂—, and

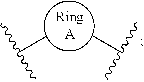

Ring A is cyclic group selected from phenyl and 5- to 6-membered heteroaryl, wherein the cyclic group is optionally substituted with 1-3 groups independently selected from C₁-C₆ alkyl;
each R^{X1} is independently selected from H, C₁-C₆ alkyl (optionally substituted with 1-3 groups independently selected from oxo, —OR^{X2}, and —N(R^{X2})₂), C₃-C₈ cycloalkyl, halogen, cyano, —OR^{X2}, and C₁-C₆ fluoroalkyl;
each R^{X2} is independently selected from H and C₁-C₆ alkyl;
each Y is independently selected from —C(R^{Y})₂—, —O—, —CO—, —NR^{YN}—, and

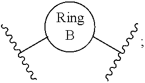

each R^{Y} is independently selected from hydrogen, hydroxy, halogen, C₁-C₆ alkyl (optionally substituted with 1-3 groups independently selected from hydroxy, C₁-C₆ alkoxy, and Q), C₃-C₈ cycloalkyl, C₆-C₁₀ aryl (optionally substituted with 1-3 groups independently selected from halogen), 5- to 10-membered heteroaryl, —OR^{Y1}, —CO₂R^{Y1}, —COR^{Y1}, —CON(R^{Y1})₂, and —N(R^{Y1})₂;
or two R^{Y}, one of which is on one atom and the second of which is on an adjacent atom, are taken together to form a pi bond;
each R is independently selected from hydrogen and C₁-C₆ alkyl, or two R^{Y1} bonded to the same nitrogen taken together form a 3- to 6-membered heterocyclyl;
each R^{YN} is independently selected from:
   H,
   C₁-C₆ alkyl, optionally substituted with 1-3 groups independently selected from:
      oxo,
      C₃-C₈ cycloalkyl (optionally substituted with 1-3 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₅ cycloalkyl (optionally substituted with 1-3 groups independently selected from halogen, C₁-C₆ alkyl, and C₁-C₆ fluoroalkyl), and
         phenyl (optionally substituted with C₁-C₆ alkyl),
      5- to 6-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         —O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN1} is independently selected from H, C₁-C₄ alkyl (optionally substituted with oxo), and C₃-C₆ cycloalkyl;
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl,
   5- to 10-membered heteroaryl, and
   3- to 6-membered heterocyclyl (optionally substituted with 1-3 groups independently selected from C₁-C₆ alkyl);
each Q is independently selected from:
   C₁-C₆ alkyl optionally substituted with 1-3 groups independently selected from:
      halogen,
      oxo,
      C₆-C₁₀ aryl (optionally substituted with 1-3 groups independently selected from halogen and —OCF₃), and
      C₃-C₈ cycloalkyl,
   C₃-C₈ cycloalkyl optionally substituted with 1-3 groups independently selected from:
      halogen,
      CN,
      C₁-C₆ alkyl (optionally substituted with 1-3 groups independently selected from halogen, —NH₂, and —NHCOMe),
      C₁-C₆ alkoxy,
      C₆-C₁₀ aryl (optionally substituted with 1-3 groups independently selected from C₁-C₆ alkyl), and
      C₃-C₈ cycloalkyl,
   C₆-C₁₀ aryl optionally substituted with 1-3 groups independently selected from:
      halogen,
      CN,
      C₁-C₆ alkyl (optionally substituted with 1-3 groups independently selected from halogen and hydroxy),
      C₁-C₆ alkoxy optionally substituted with 1-4 groups independently selected from:
         halogen,
         C₃-C₈ cycloalkyl (optionally substituted with CF₃),
      C₃-C₈ cycloalkyl (optionally substituted with 1-3 groups independently selected from halogen, CF₃, OCF₃, and C₁-C₆ alkyl), and
      C₆-C₁₀ aryl,
   5- to 10-membered heteroaryl optionally substituted with 1-3 groups independently selected from:
      halogen,
      C₁-C₆ alkyl (optionally substituted with 1-3 groups independently selected from halogen),
      C₃-C₈ cycloalkyl (optionally substituted with 1-3 CF₃ groups), and
      3- to 10-membered heterocyclyl,
   3- to 10-membered heterocyclyl optionally substituted with 1-3 groups independently selected from:
      C₁-C₆ alkyl (optionally substituted with 1-3 groups independently selected from oxo and C₃-C₈ cycloalkyl), and
      oxo;
each R¹ is independently selected from halogen, C₁-C₆ fluoroalkyl, C₁-C₆ alkyl (optionally substituted with a group selected from hydroxy, C₆-C₁₀ aryl, and 5- to 6-membered heteroaryl), —OR², —N(R²)₂, —CO₂R², —CO—N(R²)₂, —CN, C₃-C₈ cycloalkyl, C₆-C₁₀ aryl, 5- to 6-membered heteroaryl (optionally substituted with 1-3 groups independently selected from C₁-C₆ alkyl), 3- to 6-membered heterocyclyl, —B(OR²)₂, —SO₂R², —SR², —SOR², —PO(OR²)₂, and —PO(R²)₂;
each R² is independently selected from hydrogen, C₁-C₆ alkyl (optionally substituted with 1-6 groups independently selected from halogen), C₁-C₆ fluoroalkyl, and C₆-C₁₀ aryl (optionally substituted with 1-3 groups independently selected from C₁-C₆ fluoroalkyl and C₁-C₆ fluoroalkoxy);
Z is selected from

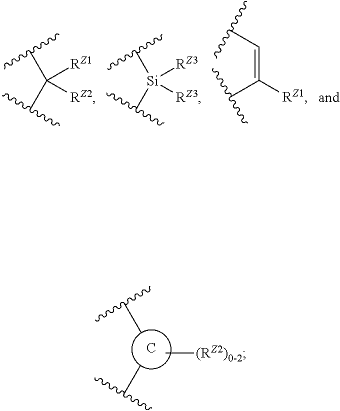

wherein Ring C is selected from C₆-C₁₀ aryl and 5- to 10-membered heteroaryl;
R^{Z1} is selected from hydrogen, —CN, C₁-C₆ alkyl (optionally substituted with 1-3 hydroxy), C₁-C₆ fluoroalkyl, 3- to 6-membered heterocyclyl, C₃-C₆ cycloalkyl, C₆-C₁₀ aryl, and 5- to 6-membered heteroaryl;
R^{Z2} is selected from hydrogen, halogen, hydroxy, NH₂, NH(CO)(C₁-C₆ alkyl), and C₁-C₆ alkoxy (optionally substituted with 1-3 groups independently selected from C₃-C₁₀ cycloalkyl),
or R^{Z1} and R^{Z2} taken together form a group selected from oxo and ═N—OH;
each R^{Z3} is independently selected from hydroxy, C₁-C₆ alkoxy, C₁-C₆ alkyl, and C₆-C₁₀ aryl; or two R^{Z3} are taken together to form a 3- to 6-membered heterocyclyl;
n is selected from 4, 5, 6, 7, and 8; and
m is selected from 0, 1, 2, and 3.
   2. The compound, deuterated derivative or pharmaceutically acceptable salt according to Embodiment 1 or Embodiment 1a, wherein Ring A is cyclic group selected from phenyl, pyrazole, and oxadiazole.
   3. The compound, deuterated derivative, or pharmaceutically acceptable salt according to any one of Embodiments 1-2, wherein each R^{X1} is independently selected from H, C₁-C₆ alkyl, halogen, cyano, —OR^{X2}, and C₁-C₆ fluoroalkyl.
   4. The compound, deuterated derivative, or pharmaceutically acceptable salt according to any one of Embodiments 1-3, wherein each R^{X2} is independently selected from H and C₁-C₄ alkyl.
   5. The compound, deuterated derivative, or pharmaceutically acceptable salt according to any one of Embodiments 1-4, wherein each R^{X1} is independently selected from H, F, —CF₃, —CH₃, —OH, —OCH₃, and CN.
   6. The compound, deuterated derivative, or pharmaceutically acceptable salt according to any one of Embodiments 1-5, wherein X is selected from: —CO—, —CH₂—,

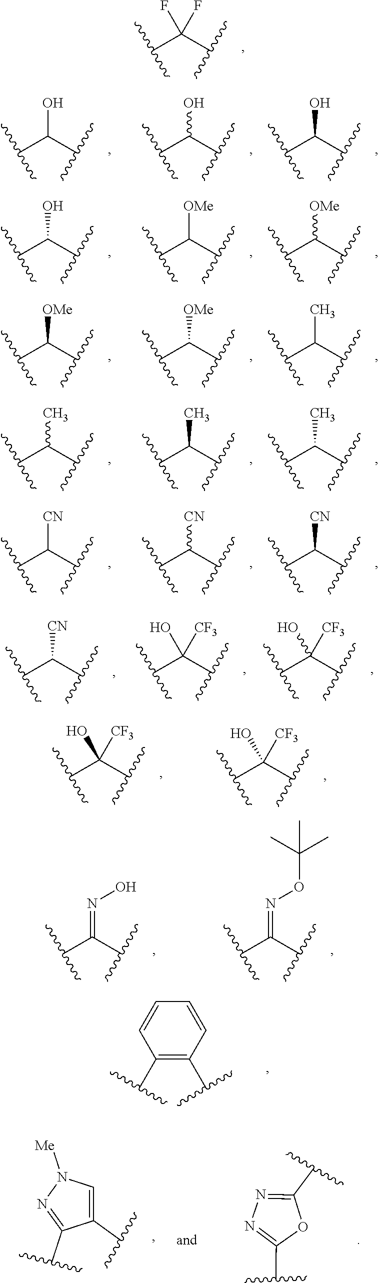

7. The compound, deuterated derivative, or pharmaceutically acceptable salt according to any one of Embodiments 1-6, wherein each Y is independently selected from —C(R^{Y})₂—, —CO—, —NR^{YN}—, and

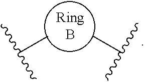

8. The compound, deuterated derivative, or pharmaceutically acceptable salt according to any one of Embodiments 1-7, wherein each R^{Y} is independently selected from hydrogen, hydroxy, halogen, and C₁-C₆ alkyl.
9. The compound, deuterated derivative, or pharmaceutically acceptable salt according to any one of Embodiments 1-8, wherein each R^{Y} is independently selected from H, —OH, —F, and —CH₃.
10. The compound, deuterated derivative, or pharmaceutically acceptable salt according to any one of Embodiments 1-9, wherein each R^{YN} is independently selected from:
H,
—CH₂CF₃,
C₁-C₆ alkyl, optionally substituted with 1-3 groups independently selected from:
   oxo,
   cyclopropyl,
   cyclobutyl optionally substituted with 1-2 fluoro groups,
   cyclohexyl optionally substituted with 1-2 fluoro groups,
   spiro[2.2]pentane,
   bicyclo[4.1.0]heptane,
   dispiro[2.0.24.13]heptane,
   phenyl optionally substituted with 1-3 groups independently selected from F, Cl, Br, CN, methyl, propyl (optionally substituted with one hydroxy), tert-butyl, neopentyl, —CHF₂, —CF₃, —C(CH₃)₂CF₃, —OCH₃, —O(2-propyl), —OCHF₂, —OCF₃, —OCF₂CHF₂, —OCH₂CF₃, cyclopropyl (optionally substituted with one CF₃), cyclobutyl (optionally substituted with 1-2 groups selected from fluoro and methyl), spiro[2.2]pentane, bicyclo[1.1.1]pentane, bicyclo[4.1.0]heptane, dispiro[2.0.24.13]heptane, phenyl, naphthyl, and

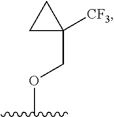 [SMILES: *OCC1(C(F)(F)F)CC1]

naphthyl,
2,2-dimethyl-2,3-dihydrobenzofuran,
2,2-difluorobenzo[d][1,3]dioxole,
tetrahydro-2H-pyran,
pyrazole optionally substituted with one group selected from methyl and propyl,
pyridine optionally substituted with one group selected from methyl, tert-butyl, CF₃, —O(cyclobutyl), —OCHF₂, cyclopropyl optionally substituted with one CF₃, and piperidine, and
pyrimidine optionally substituted with one group selected from methyl and tert-butyl,
cyclopropyl optionally substituted with 1-2 groups selected from methyl, tert-butyl, cyclopropyl, and phenyl (optionally substituted with one tert-butyl group),
cyclobutyl optionally substituted with 1-3 groups selected from fluoro, methyl, and phenyl,
cyclopentyl,
cyclohexyl optionally substituted with 1-2 groups selected from fluoro, methyl, tert-butyl, cyano, —CF₃, —CH₂NH₂, and —CH₂NHAc,
bicyclo[1.1.1]pentane optionally substituted by one group selected from fluoro, methyl, and tert-butyl,
bicyclo[2.2.1]heptane optionally substituted with one —OCH₃ group,
bicyclo[2.2.2]octane,
spiro[3.3]heptane optionally substituted with 1-2 fluoro groups,
phenyl,
tetrahydro-2H-pyran,
2-azaspiro[3.3]heptane optionally substituted with one group selected from —COMe, and —CO(cyclopropyl),
thietane 1,1-dioxide,
tetrahydro-2H-thiopyran 1,1-dioxide,
pyrazole optionally substituted with one methyl group, and
pyridine.
   11. The compound according to any one of Embodiments 1-10, wherein each R^{YN} is independently selected from: H, —CH₃,

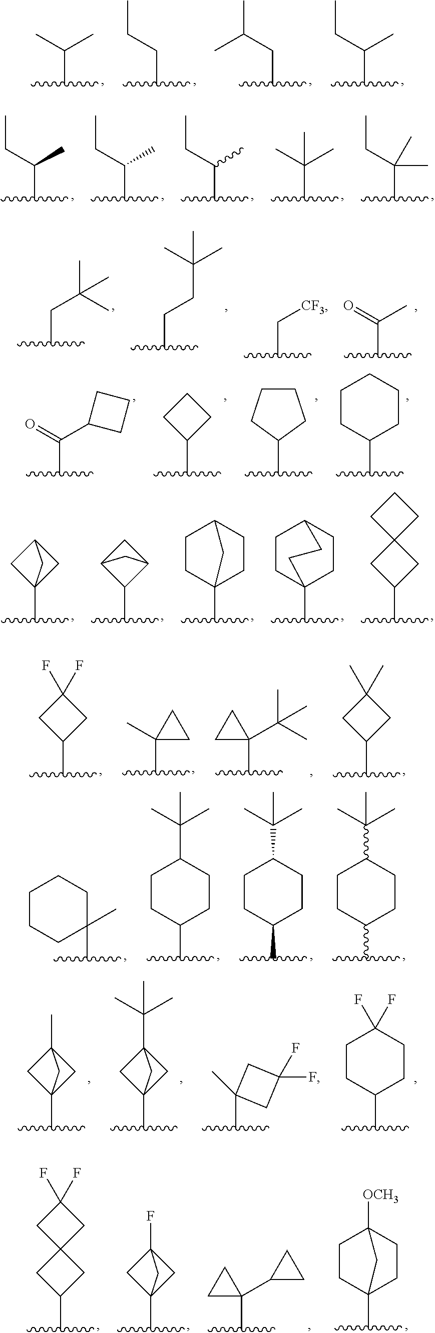

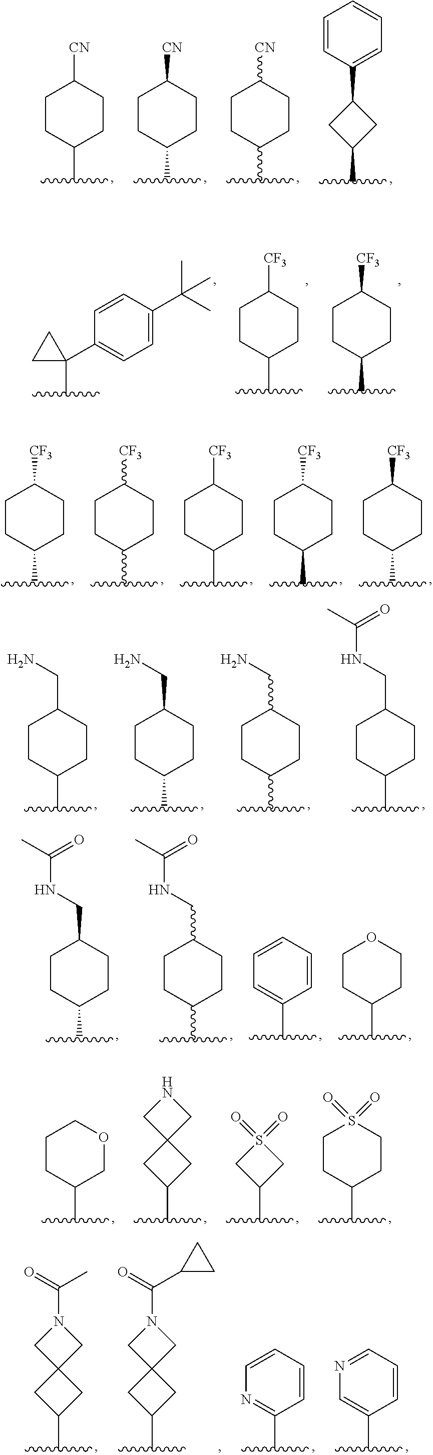

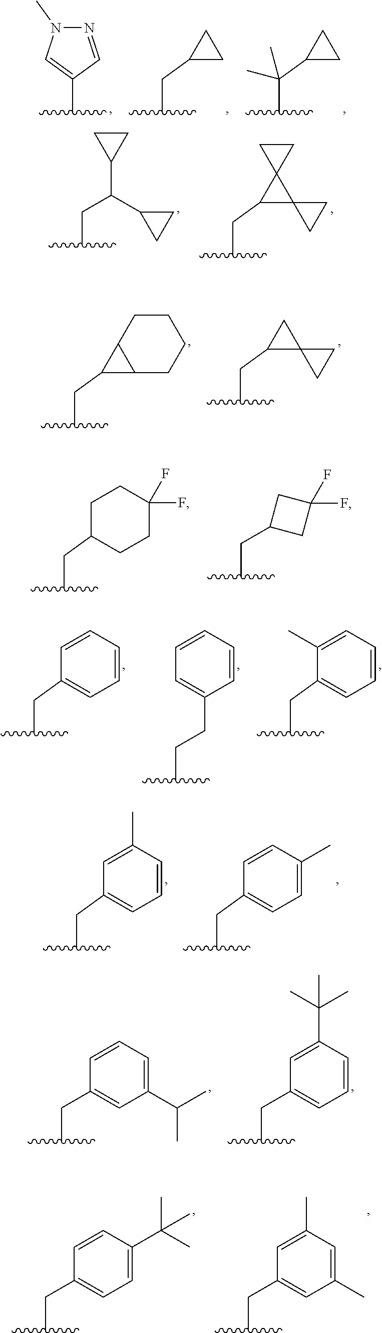

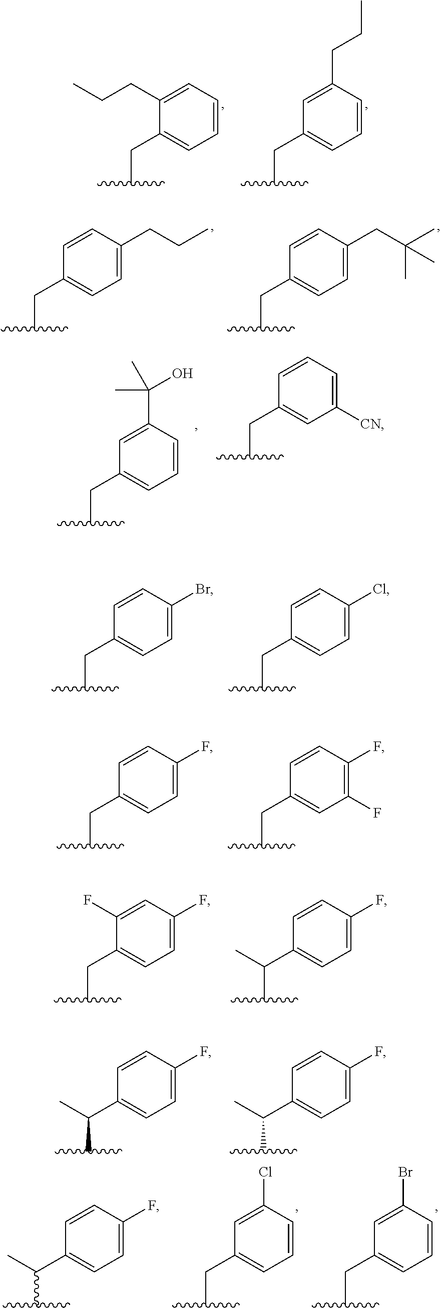

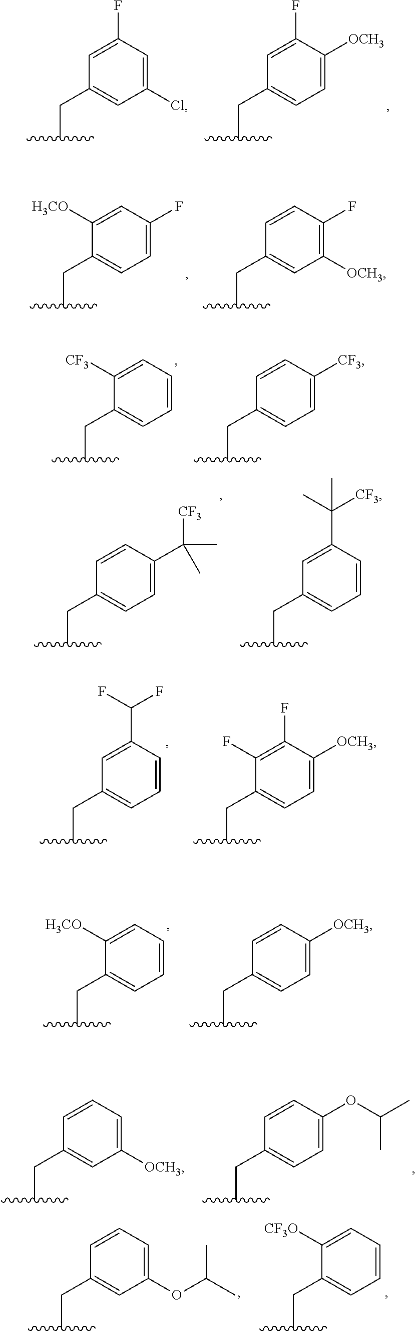

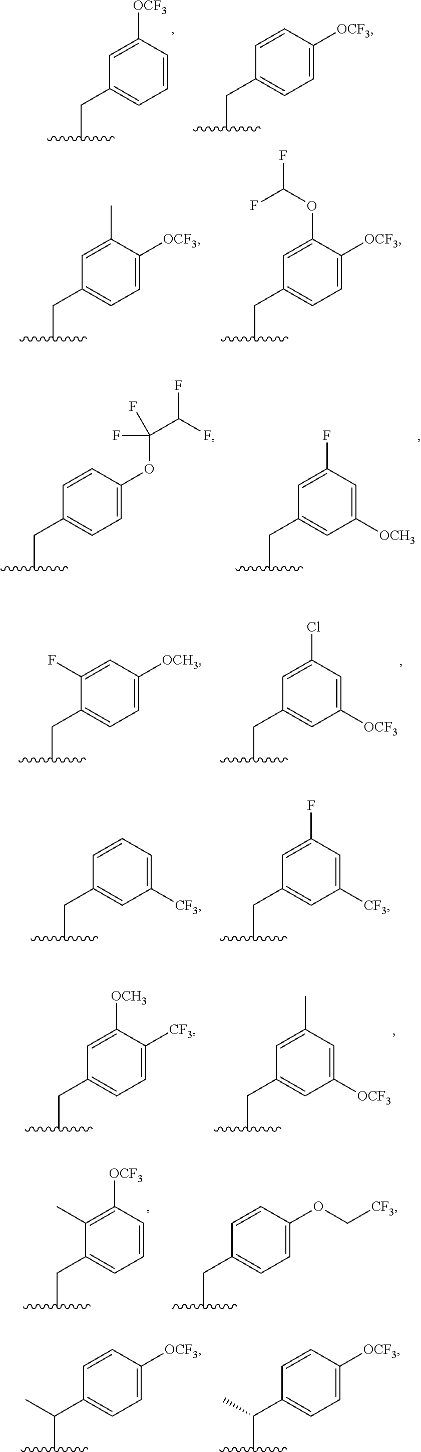

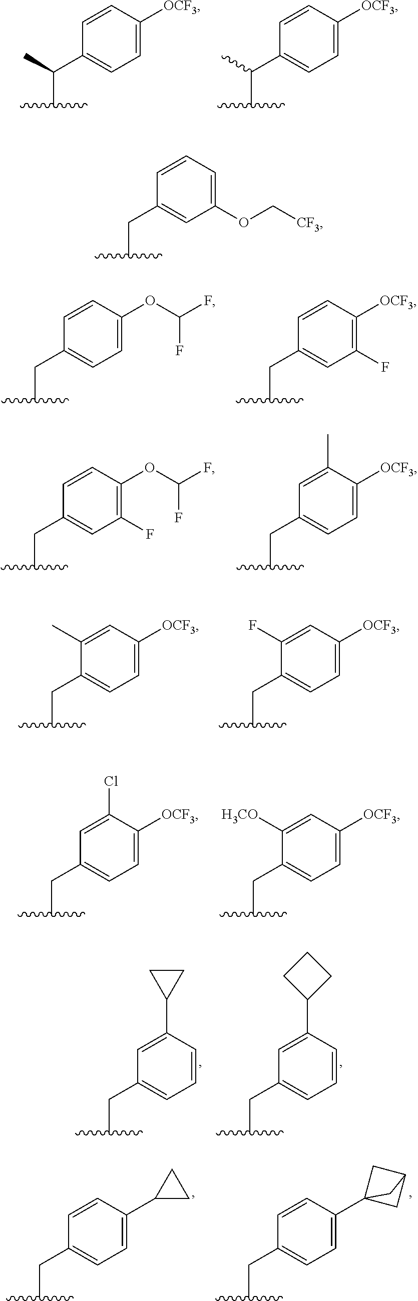

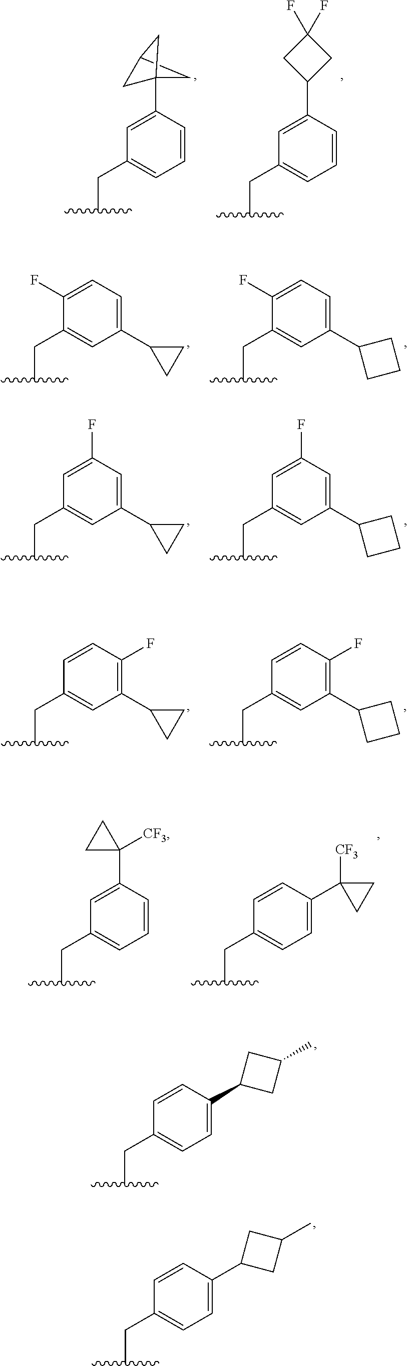

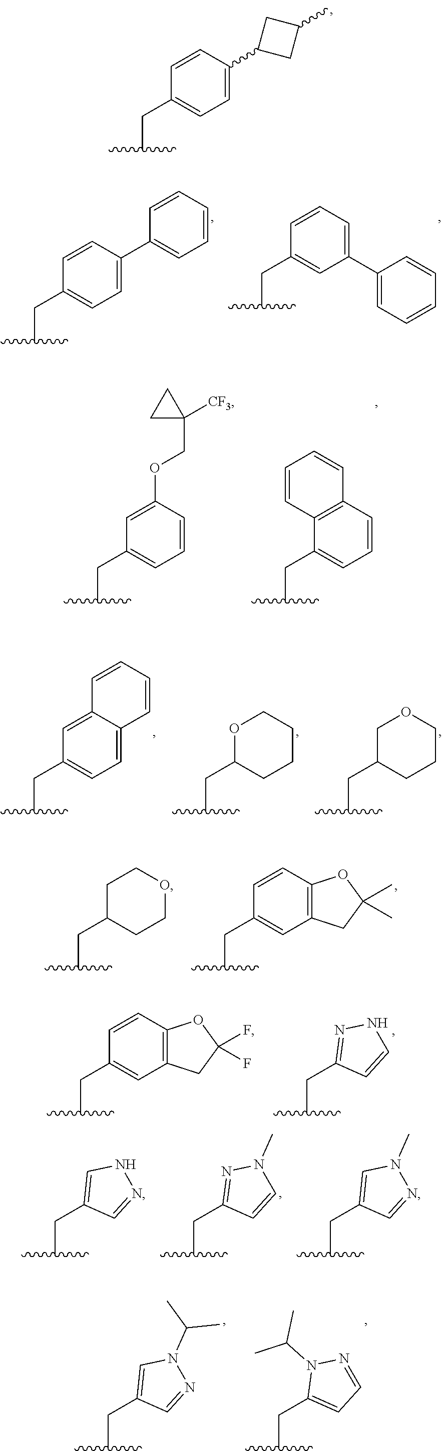

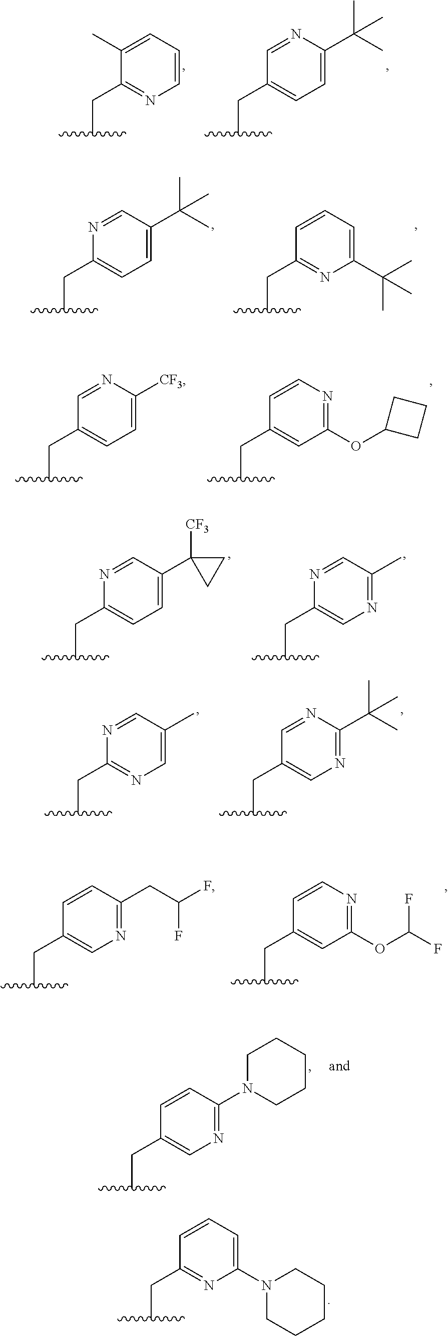

12. The compound, deuterated derivative, or pharmaceutically acceptable salt according to any one of Embodiments 1-11, wherein Ring B is selected from:
C₆-C₁₀ aryl (optionally substituted with 1-3 groups independently selected from halogen),
C₃-C₈ cycloalkyl, and
3- to 6-membered heterocyclyl (optionally substituted with 1-3 groups independently selected from C₁-C₆ alkyl).
   13. The compound, deuterated derivative, or pharmaceutically acceptable salt according to any one of Embodiments 1-12, wherein Ring B is selected from:

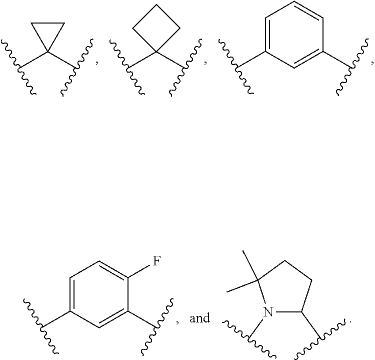

14. The compound, deuterated derivative, or pharmaceutically acceptable salt according to any one of Embodiments 1-13, wherein each R² is H.
15. The compound, deuterated derivative, or pharmaceutically acceptable salt according to any one of Embodiments 1-14, wherein each R¹ is independently selected from C₁-C₆ fluoroalkyl, —N(R²)₂, and —CN.
16. The compound, deuterated derivative, or pharmaceutically acceptable salt according to any one of Embodiments 1-15, wherein each R¹ is independently selected from —CF₃, —NH₂, and —CN.
17. The compound, deuterated derivative, or pharmaceutically acceptable salt according to any one of Embodiments 1-16, wherein R^{Z1} is selected from C₁-C₆ fluoroalkyl.
18. The compound, deuterated derivative, or pharmaceutically acceptable salt according to any one of Embodiments 1-17, wherein R^{Z2} is selected from halogen and hydroxy.
19. The compound, deuterated derivative, or pharmaceutically acceptable salt according to any one of Embodiments 1-18, wherein Z is selected from

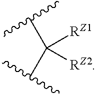

20. The compound, deuterated derivative, or pharmaceutically acceptable salt according to any one of Embodiments 1-19, wherein Z is selected from:

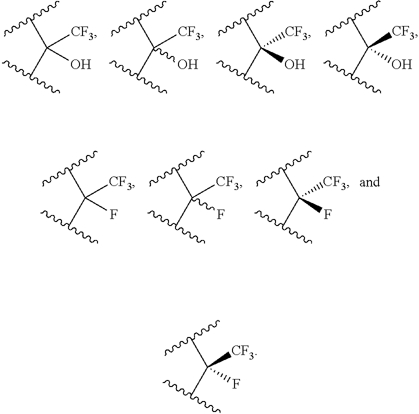

21. The compound, deuterated derivative, or pharmaceutically acceptable salt according to any one of Embodiments 1-20, wherein n is selected from 5, 6, and 7.
22. The compound, deuterated derivative, or pharmaceutically acceptable salt according to any one of Embodiment 1-21, wherein
X is selected from —CH₂— and —CO—;
each Y is independently selected from —C(R^{Y})₂—, —NR^{YN}—, and

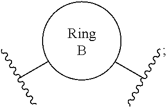

each R^{Y} is independently selected from hydrogen, halogen, and C₁-C₆ alkyl;
each R^{YN} is independently selected from:
   C₁-C₄ alkyl, optionally substituted with 1-3 groups independently selected from:
      C₆-C₁₀ aryl, optionally substituted with 1-3 groups independently selected from:
      C₁-C₆ alkyl,
      C₁-C₆ fluoroalkoxy, and
      C₃-C₈ cycloalkyl (optionally substituted with 1-3 groups independently selected from C₁-C₆ fluoroalkyl), and
   C₃-C₈ cycloalkyl optionally substituted with 1-3 groups independently selected from halogen,
Ring B is selected from C₃-C₈ cycloalkyl,
each R¹ is independently selected from C₁-C₆ fluoroalkyl and —NH₂;
Z is selected from

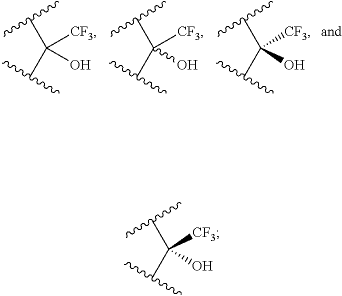

R^{Z1} is selected from C₁-C₆ fluoroalkyl;
R^{Z2} is hydroxy;
n is 6; and
m is 2.
   23. The compound, deuterated derivative, or pharmaceutically acceptable salt according to any one of Embodiments 1-22, wherein each —C(R′)₂— is independently selected from:

### —CH₂—,

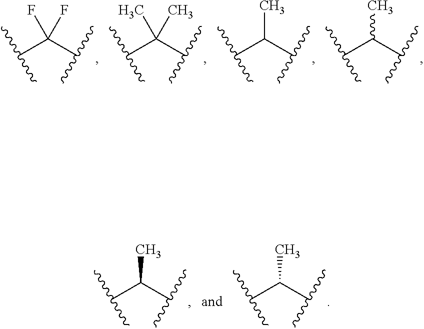

24. The compound, deuterated derivative, or pharmaceutically acceptable salt according to any one of Embodiments 1-23, wherein each wherein each —NR^{YN}— is independently selected from:

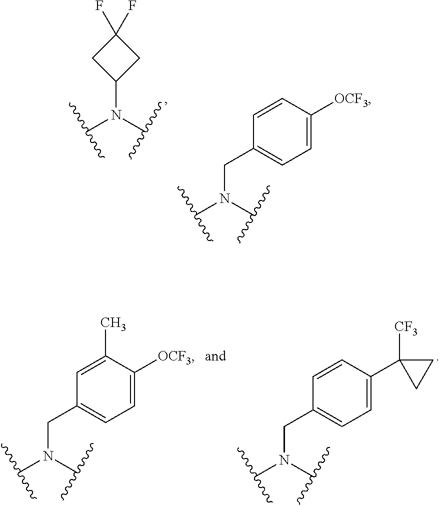 [SMILES: *N(*)C1CC(F)(F)C1 ; *N(*)Cc1ccc(OC(F)(F)F)cc1 ; *N(*)Cc1ccc(OC(F)(F)F)c(C)c1 ; *N(*)Cc1ccc(C2(C(F)(F)F)CC2)cc1]

25. The compound, deuterated derivative, or pharmaceutically acceptable salt according to any one of Embodiments 1-24, wherein Ring B is

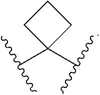 [SMILES: *C1(*)CCC1]

26. The compound, deuterated derivative, or pharmaceutically acceptable salt according to any one of Embodiments 1-25, wherein —(Y)ₙ— is a group selected from:

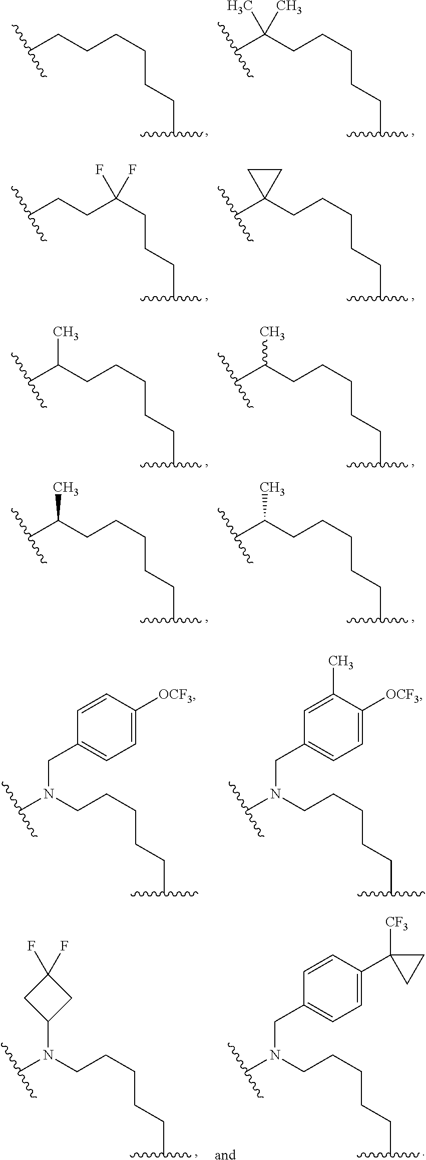

27. The compound, deuterated derivative, or pharmaceutically acceptable salt according to Embodiment 1, wherein the compound is selected from Formula IA:

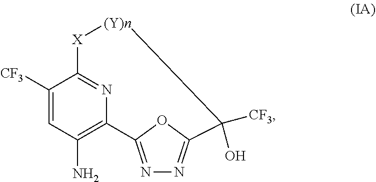

and deuterated derivatives and pharmaceutically acceptable salts thereof, wherein:
X is selected from —CH₂— and —CO—;
n is selected from 5, 6, and 7;
each Y is independently selected from —C(R^{Y})₂—, —NR^{YN}—, and

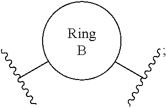

each R^{Y} is independently selected from hydrogen, hydroxy, halogen, and C₁-C₆ alkyl (optionally substituted with 1-3 groups independently selected from hydroxy, and C₁-C₆ alkoxy);
each R^{YN} is independently selected from:
   H,
   C₁-C₄ alkyl, optionally substituted with 1-3 groups independently selected from:
      oxo,
      C₃-C₈ cycloalkyl (optionally substituted with 1-3 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₈ cycloalkyl (optionally substituted with 1-3 groups independently selected from halogen, C₁-C₆ alkyl, and C₁-C₆ fluoroalkyl), and
         phenyl (optionally substituted with C₁-C₆ alkyl),
      5- to 6-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         —O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN}1 is independently selected from H, and C₁-C₄ alkyl (optionally substituted with oxo);
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl, and
   3- to 6-membered heterocyclyl (optionally substituted with 1-3 groups independently selected from C₁-C₆ alkyl);
each R¹ is independently selected from halogen, C₁-C₆ fluoroalkyl, C₁-C₆ alkyl, —N(R²)₂, and —CN; and
each R² is independently selected from hydrogen, C₁-C₃ alkyl, and C₁-C₆ fluoroalkyl.
   28. The compound, deuterated derivative, or pharmaceutically acceptable salt according to Embodiment 1, wherein the compound is selected from Formula IA′:

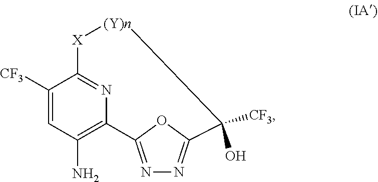

and deuterated derivatives and pharmaceutically acceptable salts thereof, wherein:
X is selected from —CH₂— and —CO—;
n is selected from 5, 6, and 7;
each Y is independently selected from —C(R^{Y})₂—, —NR^{YN}—, and

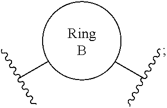

each R^{Y} is independently selected from hydrogen, hydroxy, halogen, and C₁-C₆ alkyl (optionally substituted with 1-3 groups independently selected from hydroxy, and C₁-C₆ alkoxy);
each R^{YN} is independently selected from:
   H,
   C₁-C₄ alkyl, optionally substituted with 1-3 groups independently selected from:
      oxo,
      C₃-C₈ cycloalkyl (optionally substituted with 1-3 groups independently selected from halogen and C₁-C₆ alkyl),
      C₆-C₁₀ aryl, optionally substituted with 1-3 groups independently selected from:
         halogen,
         cyano,
         C₁-C₆ alkyl (optionally substituted with 1-3 hydroxy),
         C₁-C₆ fluoroalkyl,
         C₁-C₆ alkoxy (optionally substituted with C₃-C₈ cycloalkyl, which is optionally substituted with C₁-C₆ fluoroalkyl),
         C₁-C₆ fluoroalkoxy,
         C₃-C₈ cycloalkyl (optionally substituted with 1-3 groups independently selected from halogen, C₁-C₆ alkyl, and C₁-C₆ fluoroalkyl), and
         phenyl (optionally substituted with C₁-C₆ alkyl),
      5- to 6-membered heteroaryl, optionally substituted with 1-3 groups independently selected from:
         halogen,
         C₁-C₆ alkyl,
         C₁-C₆ fluoroalkyl,
         C₁-C₆ fluoroalkoxy,
         —O₍₀₋₁₎(C₃-C₈ cycloalkyl) (optionally substituted with C₁-C₆ fluoroalkyl), and
         3- to 12-membered heterocyclyl, and
      3- to 12-membered heterocyclyl, optionally substituted with 1-3 groups selected from halogen and C₁-C₆ alkyl)
   C₁-C₆ fluoroalkyl,
   C₆-C₁₀ aryl,
   C₃-C₈ cycloalkyl optionally substituted with 1-3 groups independently selected from
      halogen,
      cyano,
      C₁-C₄ alkyl optionally substituted with 1-3 groups selected from —N(R^{YN1})₂,
      C₁-C₆ fluoroalkyl,
      C₁-C₆ alkoxy,
      C₃-C₈ cycloalkyl, and
      phenyl (optionally substituted with 1-3 groups independently selected from C₁-C₆ alkyl)
   3- to 12-membered heterocyclyl, optionally substituted with 1-3 groups selected from oxo and C₁-C₄ alkyl (optionally substituted with 1-2 groups selected from oxo and C₃-C₈ cycloalkyl),
   5- to 6-membered heteroaryl (optionally substituted with 1-3 groups selected from C₁-C₆ alkyl), and
   CO₂R^{YN1},
each R^{YN}1 is independently selected from H, and C₁-C₄ alkyl (optionally substituted with oxo);
Ring B is selected from:
   C₆-C₁₀ aryl (optionally substituted with 1-3 groups independently selected from halogen, C₁-C₆ alkyl, and C₁-C₆ alkoxy),
   C₃-C₈ cycloalkyl, and
   3- to 6-membered heterocyclyl (optionally substituted with 1-3 groups independently selected from C₁-C₆ alkyl);
each R¹ is independently selected from halogen, C₁-C₆ fluoroalkyl, C₁-C₆ alkyl,
   —N(R²)₂, and —CN; and
each R² is independently selected from hydrogen, C₁-C₃ alkyl, and C₁-C₆ fluoroalkyl.
   29. The compound, deuterated derivative, or pharmaceutically acceptable salt of Embodiment 27 or Embodiment 28, wherein n is 6.
   30. The compound, deuterated derivative, or pharmaceutically acceptable salt according to Embodiment 1, wherein the compound is selected from Formula IB:

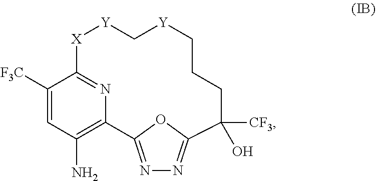

and deuterated derivatives and pharmaceutically acceptable salts thereof, wherein:
   X is selected from —CH₂— and —CO—; and
   each Y is independently selected from —CH₂—, —C(CH₃)₂—, —CH(CH₃)—, —CF₂—, cyclobutyl, and —NR^{YN}—, wherein each R^{YN} is independently selected from:
      C₁-C₄ alkyl substituted with phenyl, which is optionally substituted with 1-3 groups independently selected from C₁-C₆ alkyl, C₁-C₆ fluoroalkoxy, and C₃-C₈ cycloalkyl (optionally substituted with 1-3 groups independently selected from C₁-C₆ fluoroalkyl), and
      C₃-C₈ cycloalkyl optionally substituted with 1-3 groups independently selected from halogen.
         31. The compound, deuterated derivative, or pharmaceutically acceptable salt according to Embodiment 1, wherein the compound is selected from Formula IB′:

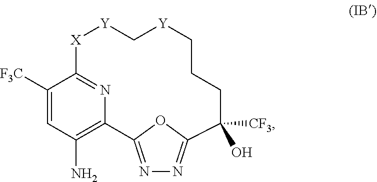

and deuterated derivatives and pharmaceutically acceptable salts thereof, wherein:
X is selected from —CH₂— and —CO—; and each Y is independently selected from —CH₂—, —C(CH₃)₂—, —CH(CH₃)—, —CF₂—, cyclobutyl, and —NR^{YN}—, wherein each R^{YN} is independently selected from:
   C₁-C₄ alkyl substituted with phenyl, which is optionally substituted with 1-3 groups independently selected from C₁-C₆ alkyl, C₁-C₆ fluoroalkoxy, and C₃-C₈ cycloalkyl (optionally substituted with 1-3 groups independently selected from C₁-C₆ fluoroalkyl), and
   C₃-C₈ cycloalkyl optionally substituted with 1-3 groups independently selected from halogen.
      32. The compound, deuterated derivative, or pharmaceutically acceptable salt of any one of Embodiments 27 to 31, wherein X is —CO—.
      33. The compound, deuterated derivative, or pharmaceutically acceptable salt of any one of Embodiments 27 to 31, wherein X is —CH₂—.
      34. A compound selected from compounds of Table 7, pharmaceutically acceptable salts thereof, and deuterated derivatives of any of the foregoing.
      35. A compound according to any one of Embodiments 1-31 or Embodiment 34, wherein the compound is selected from:

| Comp. No. | Structure |
|---|---|
| 48 | 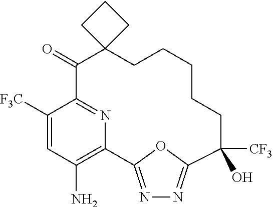 [SMILES: Nc1cc(C(F)(F)F)c2nc1-c1nnc(o1)[C@@](O)(C(F)(F)F)CCCCCC1(CCC1)C2=O] |
| 45 | 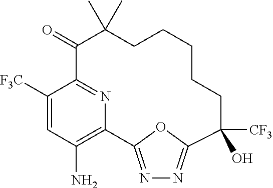 [SMILES: CC1(C)CCCCC[C@](O)(C(F)(F)F)c2nnc(o2)-c2nc(c(C(F)(F)F)cc2N)C1=O] |
| 2 | 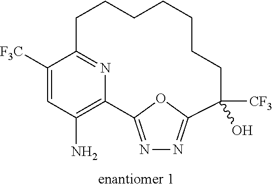 [SMILES: Nc1cc(C(F)(F)F)c2nc1-c1nnc(o1)C(O)(C(F)(F)F)CCCCCCC2] |
| 165 | 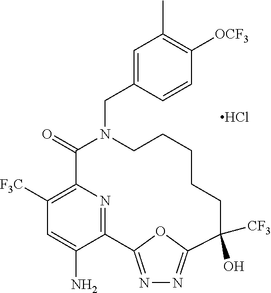 [SMILES: Cc1cc(CN2CCCCC[C@](O)(C(F)(F)F)c3nnc(o3)-c3nc(c(C(F)(F)F)cc3N)C2=O)ccc1OC(F)(F)F ; Cl] |
| 151 | 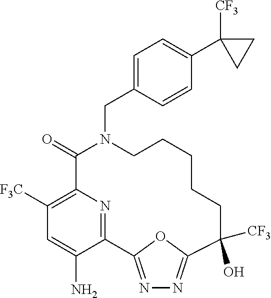 [SMILES: Nc1cc(C(F)(F)F)c2nc1-c1nnc(o1)[C@@](O)(C(F)(F)F)CCCCCN(Cc1ccc(C3(C(F)(F)F)CC3)cc1)C2=O] |
| 6 | 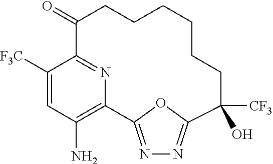 [SMILES: Nc1cc(C(F)(F)F)c2nc1-c1nnc(o1)[C@@](O)(C(F)(F)F)CCCCCCC2=O] |
| 106 | 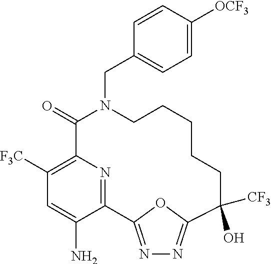 [SMILES: Nc1cc(C(F)(F)F)c2nc1-c1nnc(o1)[C@@](O)(C(F)(F)F)CCCCCN(Cc1ccc(OC(F)(F)F)cc1)C2=O] |
| 84 | 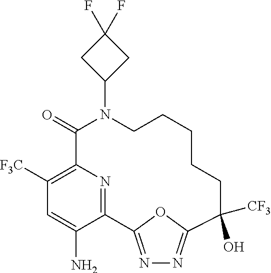 [SMILES: Nc1cc(C(F)(F)F)c2nc1-c1nnc(o1)[C@@](O)(C(F)(F)F)CCCCCN(C1CC(F)(F)C1)C2=O] |
| 38 | 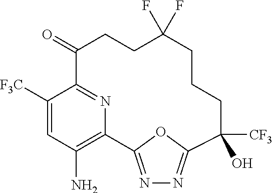 [SMILES: Nc1cc(C(F)(F)F)c2nc1-c1nnc(o1)[C@@](O)(C(F)(F)F)CCCC(F)(F)CCC2=O] |
| 50 | 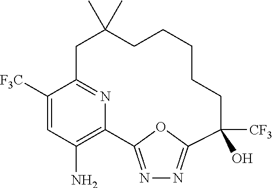 [SMILES: CC1(C)CCCCC[C@](O)(C(F)(F)F)c2nnc(o2)-c2nc(c(C(F)(F)F)cc2N)C1] |
| 56 | 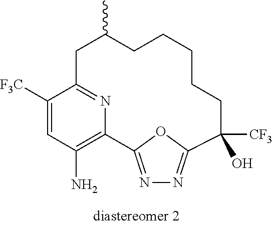 [SMILES: CC1CCCCC[C@](O)(C(F)(F)F)c2nnc(o2)-c2nc(c(C(F)(F)F)cc2N)C1] |
| 55 | 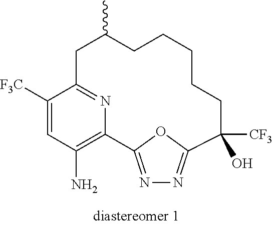 [SMILES: CC1CCCCC[C@](O)(C(F)(F)F)c2nnc(o2)-c2nc(c(C(F)(F)F)cc2N)C1] |

36. A pharmaceutical composition comprising a compound, deuterated derivative, or pharmaceutically acceptable salt of any one of Embodiments 1-35 and a pharmaceutically acceptable carrier.
37. The pharmaceutical composition according to Embodiment 36, further comprising one or more additional therapeutic agent(s).
38. The pharmaceutical composition according to Embodiment 37, wherein the one or more additional therapeutic agent(s) comprise(s) a compound with CFTR modulating activity or a salt or deuterated derivative thereof.
39. The pharmaceutical composition according to Embodiment 37 or Embodiment 38, wherein the one or more additional therapeutic agent(s) comprise(s) a CFTR corrector.
40. The pharmaceutical composition according to any one of Embodiments 37-39, wherein the one or more additional therapeutic agent(s) comprise(s) (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II):

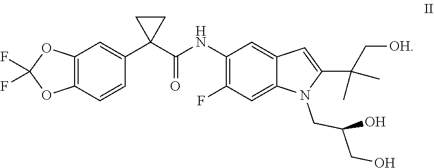 [SMILES: CC(C)(CO)c1cc2cc(NC(=O)C3(c4ccc5c(c4)OC(F)(F)O5)CC3)c(F)cc2n1C[C@@H](O)CO]

41. The pharmaceutical composition according to any one of Embodiments 37-40, wherein the one or more additional therapeutic agent(s) comprise(s) 3-(6-(1-(2,2-difluorobenzo[d][1,3]dioxol-5-yl)cyclopropanecarboxamido)-3-methylpyridin-2-yl)benzoic acid (Compound IV):

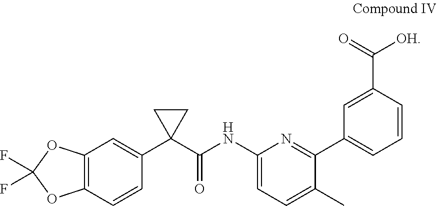 [SMILES: Cc1ccc(NC(=O)C2(c3ccc4c(c3)OC(F)(F)O4)CC2)nc1-c1cccc(C(=O)O)c1]

42. The pharmaceutical composition according to any one of Embodiments 37-41, wherein the one or more additional therapeutic agent(s) comprise(s) N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound V):

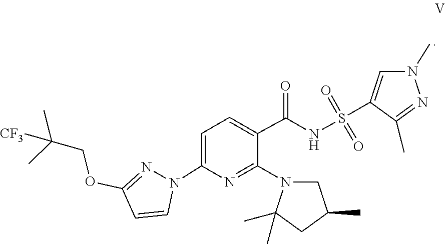 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]

43. The pharmaceutical composition according to any one of Embodiments 37-42, wherein the one or more additional therapeutic agent(s) comprise(s) N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide (Compound VI):

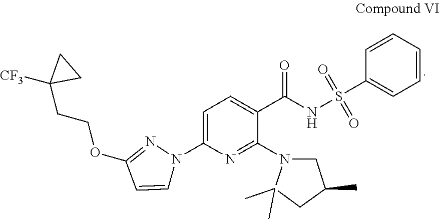 [SMILES: C[C@@H]1CN(c2nc(-n3ccc(OCCC4(C(F)(F)F)CC4)n3)ccc2C(=O)NS(=O)(=O)c2ccccc2)C(C)(C)C1]

44. The pharmaceutical composition according to any one of Embodiments 37-43, wherein the one or more additional therapeutic agent(s) comprise(s) (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound VII):

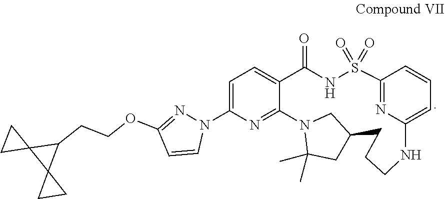 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]

45. The pharmaceutical composition according to any one of Embodiments 37-44, wherein the one or more additional therapeutic agent(s) comprise(s) (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound VIII):

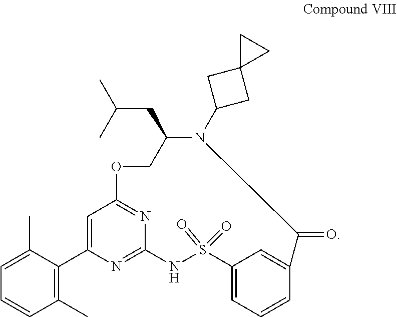 [SMILES: Cc1cccc(C)c1-c1cc2nc(n1)NS(=O)(=O)c1cccc(c1)C(=O)N(C1CC3(CC3)C1)[C@H](CC(C)C)CO2]

46. The pharmaceutical composition according to any one of Embodiments 37-45, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, FDL-169, ARN5562, ARN21586, ARN22081, ARN22652, ARN23765, ARN23766, and PTI-801.
47. The pharmaceutical composition according to any one of Embodiments 37-46, wherein the one or more additional therapeutic agent(s) comprise(s) a CFTR potentiator enhancer.
48. The pharmaceutical composition according to any one of Embodiments 37-47, wherein the one or more additional therapeutic agent(s) comprise(s) ASP-11.
49. The pharmaceutical composition according to any one of Embodiments 37-48, wherein the one or more additional therapeutic agent(s) comprise(s) a CFTR potentiator.
50. The pharmaceutical composition according to any one of Embodiments 37-49, wherein the one or more additional therapeutic agent(s) comprise(s) a compound selected from N-(5-hydroxy-2,4-di-tert-butyl-phenyl)-4-oxo-1H-quinoline-3-carboxamide (Compound III):

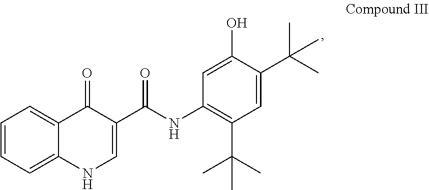 [SMILES: CC(C)(C)c1cc(C(C)(C)C)c(NC(=O)c2c[nH]c3ccccc3c2=O)cc1O]

and N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound III-d):

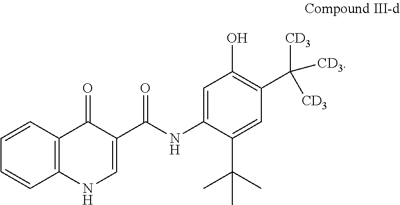 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

51. The pharmaceutical composition according to any one of Embodiments 37-50, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
52. The pharmaceutical composition according to any one of Embodiments 37-51, wherein the one or more additional therapeutic agent(s) comprise(s) a CFTR amplifier.
53. The pharmaceutical composition according to any one of Embodiments 37-52, wherein the one or more additional therapeutic agent(s) comprise(s) PTI-428.
54. The pharmaceutical composition according to any one of Embodiments 37-53, wherein the one or more additional therapeutic agent(s) comprise(s) a CFTR readthrough agent.
55. The pharmaceutical composition according to any one of Embodiments 37-54, wherein the one or more additional therapeutic agent(s) comprise(s) ELX-02.
56. The pharmaceutical composition according to any one of Embodiments 37-55, wherein the one or more additional therapeutic agent(s) comprise(s) a nucleic acid therapy.
57. The pharmaceutical composition according to any one of Embodiments 37-56, wherein the one or more additional therapeutic agent(s) comprise(s) at least one agent selected from MRT5005, Lunar-CF, and RCT223.
58. The pharmaceutical composition according to any one of Embodiments 37-57, wherein the one or more additional therapeutic agent(s) comprise(s) an ENaC inhibitor.
59. The pharmaceutical composition according to any one of Embodiments 37-58, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, and ARO-ENaC1001.
60. The pharmaceutical composition according to any one of Embodiments 37-59, wherein the one or more additional therapeutic agent(s) comprise(s) a TMEM16A modulator.
61. The pharmaceutical composition according to any one of Embodiments 37-60, wherein the one or more additional therapeutic agent(s) comprise(s) ETD002.
62. The pharmaceutical composition according to any one of Embodiments 37-61, wherein the one or more additional therapeutic agent(s) comprise(s) a GPR39 Agonist.
63. The pharmaceutical composition according to any one of Embodiments 37-62, wherein the one or more additional therapeutic agent(s) comprise(s) DS-1039.
64. A method of treating cystic fibrosis, comprising administering an effective amount of the compound, salt, or deuterated derivative according to any one of Embodiments 1-35 or the pharmaceutical composition according to any one of Embodiments 36-63 to a patient in need thereof.
65. The method according to Embodiment 64, further comprising administering one or more additional therapeutic agent(s).
66. The method according to Embodiment 65, wherein the one or more additional therapeutic agent(s) comprise(s) a compound with CFTR modulating activity or a salt or deuterated derivative thereof.
67. The method according to Embodiment 65 or Embodiment 66, wherein the one or more additional therapeutic agent(s) comprise(s) a CFTR corrector.
68. The method according to any one of Embodiments 65-67, wherein the one or more additional therapeutic agent(s) comprise(s) Compound II.
69. The method according to any one of Embodiments 65-68, wherein the one or more additional therapeutic agent(s) comprise(s) Compound IV.
70. The method according to any one of Embodiments 65-69, wherein the one or more additional therapeutic agent(s) comprise(s) Compound V.
71. The method according to any one of Embodiments 65-70, wherein the one or more additional therapeutic agent(s) comprise(s) Compound VI.
72. The method according to any one of Embodiments 65-71, wherein the one or more additional therapeutic agent(s) comprise(s) Compound VII.
73. The method according to any one of Embodiments 65-72, wherein the one or more additional therapeutic agent(s) comprise(s) Compound VIII.
74. The method according to any one of Embodiments 65-73, wherein the one or more additional therapeutic agent(s) comprise(s) at least one compound selected from PTI-428, ABBV-2222, ABBV-2851, GLPG2737, ABBV-3221, ABBV-3748, ABBV-3903, ABBV-119, ABBV-2851, FDL-169, ARN5562, ARN21586, ARN22081, ARN22652, ARN23765, ARN23766, and PTI-801.
75. The method according to any one of Embodiments 65-74, wherein the one or more additional therapeutic agent(s) comprise(s) a CFTR potentiator enhancer.
76. The method according to any one of Embodiments 65-75, wherein the one or more additional therapeutic agent(s) comprise(s) ASP-11.
77. The method according to any one of Embodiments 65-76, wherein the one or more additional therapeutic agent(s) comprise(s) a CFTR potentiator.
78. The method according to any one of Embodiments 65-77, wherein the one or more additional therapeutic agent(s) comprise(s) a compound selected from Compound III and Compound III-d.
79. The method according to any one of Embodiments 65-78, wherein the one or more additional therapeutic agent(s) comprise(s) at least one compound selected from FDL-176, PTI-808, GLPG1837/ABBV-974, GLPG2451/ABBV-2451, QBW251 (Icenticaftor), GLPG3067/ABBV-3067 (Navocaftor), and ABBV-191.
80. The method according to any one of Embodiments 65-79, wherein the one or more additional therapeutic agent(s) comprise(s) a CFTR amplifier.
81. The method according to any one of Embodiments 65-80, wherein the one or more additional therapeutic agent(s) comprise(s) PTI-428.
82. The method according to any one of Embodiments 65-81, wherein the one or more additional therapeutic agent(s) comprise(s) a CFTR readthrough agent.
83. The method according to any one of Embodiments 65-82, wherein the one or more additional therapeutic agent(s) comprise(s) ELX-02.
84. The method according to any one of Embodiments 65-83, wherein the one or more additional therapeutic agent(s) comprise(s) a nucleic acid therapy.
85. The method according to any one of Embodiments 65-84, wherein the one or more additional therapeutic agent(s) comprise(s) at least one agent selected from MRT5005, Lunar-CF, and RCT223.
86. The method according to any one of Embodiments 65-85, wherein the one or more additional therapeutic agent(s) comprise(s) an ENaC inhibitor.
87. The method according to any one of Embodiments 65-86, wherein the one or more additional therapeutic agent(s) comprise(s) amiloride, ETD001, CF552, GS-9411, GS-5737, P-1037 (VX-371), P-1055 (VX-551), AZD5634, SPX-101, Ionis-ENaC-2.5 Rx, BI 1265162, and ARO-ENaC1001.
88. The method according to any one of Embodiments 65-87, wherein the one or more additional therapeutic agent(s) comprise(s) a TMEM16A modulator.
89. The method according to any one of Embodiments 65-88, wherein the one or more additional therapeutic agent(s) comprise(s) ETD002.
90. The method according to any one of Embodiments 65-89, wherein the one or more additional therapeutic agent(s) comprise(s) a GPR39 Agonist.
91. The method according to any one of Embodiments 65-90, wherein the one or more additional therapeutic agent(s) comprise(s) DS-1039.
92. The compound, salt, or deuterated derivative of any one of Embodiments 1-35 or the pharmaceutical composition according to any one of Embodiments 36-63 for use in the treatment of cystic fibrosis.
93. Use of the compound, salt, or deuterated derivative of any one of Embodiments 1-35 or the pharmaceutical composition according to any one of Embodiments 36-63 in the manufacture of a medicament for the treatment of cystic fibrosis.

### Examples

General Experimental Procedures

### Abbreviations

AcOH: Acetic acid
Boc anhydride ((Boc)₂O): Di-tert-butyl dicarbonate
Boc: Butoxy carbonyl
BOP: Benzotriazol-1-yloxytris(dimethylamino)phosphonium hexafluorophosphate
t-BuOH: tert-Butanol
CDCl₃: Chloroform-d
CDI: 1,1′-Carbonyldiimidazole
CD₃OD: Methyl-d4 alcohol-d
CH₂Cl₂: Dichloromethane
CH₃CN: Acetonitrile
CO₂: Carbon dioxide
Cs₂CO₃: Cesium carbonate
CuI: Copper(I) iodide
DCE: 1,2-Dichloroethane
DCM: Dichloromethane
DIEA: (DIPEA; N,N-Diisopropylethylamine)
DMAP: 4-Dimethylaminopyridine
DMF: N,N-Dimethylformamide
DMP: Dess-Martin Periodinane
DMSO: Dimethyl sulfoxide
DMSO-d6: Dimethyl sulfoxide-d6
EDCI: 1-Ethyl-3-(3-dimethylaminopropyl)carbodiimide
ESI-MS: Electrospray ionization mass spectrometry
Et₂O: Diethyl ether
Et₃N or TEA: Triethylamine
EtOAc: Ethyl acetate
EtOH: Ethanol
Et₂O: Diethyl ether
ESI-MS: Electrospray ionization mass spectrometry
Grubbs catalyst 2nd Generation: [1,3-Bis(2,4,6-trimethylphenyl)imidazolidin-2-ylidene]-dichloro-[(2-isopropoxyphenyl)methylene]ruthenium
H₂: Hydrogen
HATU: N-[(Dimethylamino)-1H-1,2,3-triazolo-[4,5-b]pyridin-1-ylmethylene]-N-methylmethanaminium hexafluorophosphate N-oxide
HCl: Hydrochloric acid
HOBT: Hydroxybenzotriazole
HPLC: High performance liquid chromatography
H₂: Hydrogen
H₂O₂: Hydrogen peroxide
KHSO₄: Potassium bisulfate
KOH: Potassium hydroxide
K₂CO₃: Potassium carbonate
KMnO₄: Potassium permanganate
KHCO₃: Potassium bicarbonate
LC: Liquid chromatography
LiAlH₄: Lithium aluminum hydride
LiOH: Lithium hydroxide
MeMgCl: Methyl magnesium chloride
MeTHF or 2-MeTHF or 2-Me-THF: 2-Methyltetrahydrofuran
MeOH: Methanol
MTBE: Methyl tert-butyl ether
MgSO₄: Magnesium sulfate
MS: Mass spectrometry
n-Bu₄NF·H₂O: Tetra-n-butylammonium fluoride monohydrate
Na: Sodium
NaH: Sodium hydride
NaHCO₃: Sodium bicarbonate
NaOAc: Sodium acetate
NaOH: Sodium hydroxide
Na₂SO₄: Sodium sulfate
NBS: N-bromosuccinimide
NH₃: Ammonia
NH₄Cl: Ammonium chloride
NH₄HCO₃: Ammonium bicarbonate
NMP: N-Methyl-2-pyrrolidone
NMR: Nuclear magnetic resonance
N2: Nitrogen
Pd/C: Palladium on carbon
Pd₂(dba)₃: Tris(dibenzylideneacetone)dipalladium(0)
Pd(dppf)Cl₂: 1,1′-Bis(diphenylphosphino)ferrocene palladium(II) chloride
Pd(OAc)₂: Palladium(II) acetate
PhI(OAc)₂: (Diacetoxyiodo)benzene
POCl₃: Phosphoryl chloride
PtO₂: Platinum oxide
RT or rt: Room temperature
SFC: Supercritical fluid chromatography
Silica Cat Pd: Palladium on silica
SiO₂: Silica gel
TBAF: Tetra-n-butylammonium fluoride
TBAI: Tetrabutylammonium iodide
TBDPS-Cl or TBDPSCl: tert-Butyldiphenylchlorosilane
TEA: Triethylamine
TEMPO: 2,2,6,6-Tetramethylpiperidinyloxy
TFA: Trifluoroacetic acid
THF: Tetrahydrofuran
Ti(OEt)₄: Titanium (IV)ethoxide
TMEDA: Tetramethylethylenediamine
TMSCF₃: Trifluoromethyltrimethylsilane
p-TsCl or tosyl chloride: p-Toluenesulfonyl chloride or 4-Toluenesulfonyl chloride
T3P: 1-Propanephosphonic anhydride
UPLC: Ultra Performance Liquid Chromatography
Xantphos: 4,5-Bis(diphenylphosphino)-9,9-dimethylxanthene
Zhan catalyst-1B: Dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II)

### General UPLC-MS/HPLC-MS/GC Analytical Methods

LC Method A: Analytical reverse phase UPLC-MS using an Acquity UPLC-MS BEH C₁₈ column (50×2.1 mm, 1.7 m particle size) made by Waters (pn: 186002350), and a dual gradient run from 1% to 99% mobile phase B over 3.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method B: Analytical reverse phase HPLC-MS using a Kinetex C₁₈ column (4.6×50 mm, 2.6 μm particle size). Temp: 45° C.; Flow: 2.0 mL/min; Run Time: 3 min. Mobile phase: Initial 95% water (+0.1% formic acid) and 5% acetonitrile (+0.1% formic acid) linear gradient to 95% acetonitrile (+0.1% formic acid) for 2.0 min then hold at 95% acetonitrile (+0.1% formic acid) for 1.0 min.

LC Method C: Analytical reverse phase HPLC-MS using a Kinetex Polar C₁₈ column (3.0×50 mm, 2.6 μm particle size), Temp: 45° C.; Flow: 1.2 mL/min; Run time: 6 min. Mobile phase: Initial 95% water (+0.1% formic acid) and 5% acetonitrile (+0.1% formic acid) linear gradient to 95% acetonitrile (+0.1% formic acid) for 4.0 min then hold at 95% acetonitrile (+0.1% formic acid) for 2.0 min.

LC Method D: Analytical reverse phase UPLC-MS using an Acquity UPLC-MS BEH C₁₈ column (50×2.1 mm, 1.7 m particle size) made by Waters (pn: 186002350), and a dual gradient run from 1% to 99% mobile phase B over 5.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method E: Analytical reverse phase HPLC-MS using a Kinetex Polar C₁₈ column (3.0×50 mm, 2.6 μm particle size), Temp: 45° C.; Flow: 1.2 mL/min; Run time: 3 min. Mobile phase: Initial 95% water (+0.1% formic acid) and 5% acetonitrile (+0.1% formic acid) linear gradient to 95% acetonitrile (+0.1% formic acid) for 2.0 min then hold at 95% acetonitrile (+0.1% formic acid) for 1.0 min.

LC Method F: Analytical reverse phase HPLC-MS using a Kinetex C₁₈ column (4.6×50 mm, 2.6 μm particle size), Temp: 45° C.; Flow: 2.0 mL/min; Run Time: 6 min. Mobile phase: Initial 95% water (+0.1% formic acid) and 5% acetonitrile (+0.1% formic acid) linear gradient to 95% acetonitrile (+0.1% formic acid) for 4.0 min then hold at 95% acetonitrile (+0.1% formic acid) for 2.0 min.

LC Method G: Analytical reverse phase HPLC-MS using a Merckmillipore Chromolith SpeedROD C₁₈ column (50×4.6 mm) and a dual gradient run from 5% to 100% mobile phase B over 6 minutes. Mobile phase A=water (+0.1% trifluoroacetic acid). Mobile phase B=acetonitrile (+0.1% trifluoroacetic acid).

LC Method H: Analytical reverse phase HPLC-MS using a Waters Cortex C₁₈ column (3.0×50 mm, 2.7 μm particle size) made by Waters (pn: 186007370), Temp: 55° C.; Flow: 1.2 mL/min; Mobile phase A: Water (+0.1% trifluoroacetic acid). Mobile phase B: Acetonitrile (+0.1% trifluoroacetic acid). Gradient: 5% to 100% B over 4 min, with equilibration at 100% B for 0.5 min, equilibration to 5% B over 1.5 min.

LC Method I: Analytical reverse phase HPLC-MS using a Kinetex Polar C₁₈ column (3.0×50 mm, 2.6 μm particle size), Temp: 45° C.; Flow: 1.0 mL/min; Run time: 6 min. Mobile phase: Initial 98% water (+0.1% formic acid) and 2% acetonitrile (+0.1% formic acid) hold 0.5 min then linear gradient to 95% acetonitrile (+0.1% formic acid) for 3.0 min then hold at 95% acetonitrile (+0.1% formic acid) for 2.5 min.

LC Method J: Analytical reverse phase UPLC-MS using an Acquity UPLC-MS BEH C₁₈ column (50×2.1 mm, 1.7 m particle size) made by Waters (pn: 186002350), and a dual gradient run from 30% to 99% mobile phase B over 3.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method K: Analytical reverse phase HPLC-MS using a Kinetex EVO C₁₈ column (4.6×50 mm, 2.6 μm particle size), Temp: 45° C.; Flow: 2.0 mL/min; Run time: 4 min. Mobile phase: Initial 95% water (+0.1% formic acid) and 5% acetonitrile (+0.1% formic acid) linear gradient to 95% acetonitrile (+0.1% formic acid) for 2.0 min then hold at 95% acetonitrile (+0.1% formic acid) for 2.0 min.

LC Method L: Analytical reverse phase HPLC-MS using an X-Terra MS C₁₈ column (4.6×150 mm, 5 μm particle size), Temperature: 40° C.; Flow: 1.5 mL/min; Run Time: 10 min. Mobile phase: Initial 95% water (+10 mM ammonium bicarbonate) and 5% acetonitrile linear gradient to 95% acetonitrile for 6.5 min then hold at 95% acetonitrile for 3.5 min.

LC Method M: Analytical reverse phase UPLC-MS using an Acquity UPLC-MS BEH C₁₈ column (50×2.1 mm, 1.7 m particle size) made by Waters (pn: 186002350), and a dual gradient run from 50% to 99% mobile phase B over 3.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method N: Analytical reverse phase UPLC-MS using an Acquity UPLC-MS BEH C₁₈ column (30×2.1 mm, 1.7 m particle size) made by Waters (pn: 186002349), and a dual gradient run from 1% to 99% mobile phase B over 2.9 minutes. Mobile phase A=water (+5 mM ammonium hydroxide). Mobile phase B=acetonitrile. Flow rate=1.5 mL/min, injection volume=1.5 μL, and column temperature=60° C.

LC Method O: Analytical reverse phase HPLC-MS using a Kinetex Polar C₁₈ column (3.0×50 mm, 2.6 μm particle size), Temp: 45° C.; Flow: 1.2 mL/min; Run time: 4 min. Mobile phase: Initial 95% water (+0.1% formic acid) and 5% acetonitrile (+0.1% formic acid) linear gradient to 95% acetonitrile (+0.1% formic acid) for 3.0 min then hold at 95% acetonitrile (+0.1% formic acid) for 1.0 min.

LC Method P: Analytical reverse phase UPLC-MS using an Acquity UPLC-MS BEH C₁₈ column (100×2.1 mm, 1.7 m particle size) made by Waters (pn: 186002352), and a dual gradient run from 1% to 99% mobile phase B over 13.5 minutes. Mobile phase A=water (+0.05% trifluoroacetic acid). Mobile phase B=acetonitrile (+0.035% trifluoroacetic acid). Flow rate=0.8 mL/min, injection volume=1.5 μL, and column temperature=60° C.

LC Method Q: Analytical reverse phase HPLC-MS using an Onyx Monolithic C₁₈ column (50×4.6 mm) sold by Phenomenex (pn: CH0-7644), and a dual gradient run from 1% to 99% mobile phase B over 2.9 minutes. Mobile phase A=water (+0.05% trifluoroacetic acid). Mobile phase B=acetonitrile (+0.035% trifluoroacetic acid). Flow rate=12 mL/min, injection volume=50 μL, and column temperature=25° C.

LC Method R: Analytical reverse phase UPLC-MS using an Acquity UPLC-MS BEH C₁₈ column (30×2.1 mm, 1.7 m particle size) made by Waters (pn: 186002349), and a dual gradient run from 30% to 99% mobile phase B over 1.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method S: Analytical reverse phase UPLC-MS using an Acquity UPLC-MS BEH C₁₈ column (30×2.1 mm, 1.7 m particle size) made by Waters (pn: 186002349), and a dual gradient run from 1% to 99% mobile phase B over 1.0 minutes. Mobile phase A=water (+0.05% trifluoroacetic acid). Mobile phase B=acetonitrile (+0.035% trifluoroacetic acid). Flow rate=1.5 mL/min, injection volume=1.5 μL, and column temperature=60° C.

LC Method T: Analytical reverse phase UPLC-MS using an Acquity UPLC-MS BEH C₁₈ column (30×2.1 mm, 1.7 m particle size) made by Waters (pn: 186002349), and a dual gradient run from 50% to 99% mobile phase B over 1.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method U: Analytical reverse phase UPLC-MS using an Acquity UPLC-MS BEH C₁₈ column (30×2.1 mm, 1.7 m particle size) made by Waters (pn: 186002349), and a dual gradient run from 75% to 99% mobile phase B over 1.0 minutes. Mobile phase A=water (+0.05% trifluoroacetic acid). Mobile phase B=acetonitrile (+0.035% trifluoroacetic acid). Flow rate=1.2 mL/min, injection volume=1.5 μL, and column temperature=60° C.

LC Method V: Analytical reverse phase HPLC-MS using a Kinetex EVO C₁₈ column (2.1×50 mm 2.6 μm particle size), Temp: 45° C.; Flow: 1.0 mL/min; Run time: 1.5 min. Mobile phase: Initial 98% of mobile phase A (10 mM ammonium formate in water:acetonitrile, 95:5, pH 9) and 2% mobile phase B (acetonitrile) linear gradient to 98% acetonitrile for 1.15 min then hold at 98% acetonitrile for 0.2 min then return to 98% water and 10 mM ammonium formate for 0.05 min and hold for 0.1 min.

LC Method W: Analytical reverse phase HPLC-MS using an XBridge C₁₈ column (3.0×30 mm, 5 μm particle size); Flow: 2.0 mL/min; Run time: 3 min. Mobile phase: Initial 95% water (+10 mM ammonium bicarbonate) and 5% acetonitrile to 5% water (+10 mM ammonium bicarbonate) and 95% acetonitrile over 2 min then hold at 95% acetonitrile and 5% water (+10 mM ammonium bicarbonate) for 1 min.

LC Method X: Analytical reverse phase HPLC-MS using a Kinetex Polar C₁₈ column (3.0×50 mm, 2.6 μm particle size), Temp: 45° C.; Flow: 1.2 mL/min; Run time: 5 min. Mobile phase: Initial 95% water (+0.1% formic acid) and 5% acetonitrile (+0.1% formic acid) linear gradient to 95% acetonitrile (+0.1% formic acid) for 4.0 min then hold at 95% acetonitrile (+0.1% formic acid) for 1.0 min.

LC Method Y: Analytical reverse phase HPLC-MS using a Luna C₁₈ column (3.0×50 mm, 3 μm particle size), Temp: 45° C.; Flow: 1.5 mL/min; Run time: 3.5 min. Mobile phase: Initial 95% water (+0.1% formic acid) and 5% acetonitrile (+0.1% formic acid) linear gradient to 95% acetonitrile (+0.1% formic acid) for 1.3 min then hold at 95% acetonitrile (+0.1% formic acid) for 2.2 min.

LC Method Z: Analytical reverse phase HPLC-MS using a Luna C₁₈ column (3.0×50 mm, 3 μm particle size), Temp: 45° C.; Flow: 1.5 mL/min; Run time: 2.5 min. Mobile phase: Initial 95% water (+0.1% formic acid) and 5% acetonitrile (+0.1% formic acid) linear gradient to 95% acetonitrile (+0.1% formic acid) for 1.3 min then hold at 95% acetonitrile (+0.1% formic acid) for 1.2 min.

LC Method AA: Analytical reverse phase HPLC-MS using a SunFire C₁₈ column (4.6×75 mm, 3.5 μm particle size), Temp: 45° C.; Flow: 1.5 mL/min; Run time: 6 min. Mobile phase: Initial 95% water (+0.1% formic acid) and 5% acetonitrile (+0.1% formic acid) linear gradient to 95% acetonitrile (+0.1% formic acid) for 4.0 min then hold at 95% acetonitrile (+0.1% formic acid) for 2.0 min.

LC Method BB: Analytical reverse phase HPLC-MS using an XBridge C₁₈ column (4.6×75 mm, 5 μm particle size); Flow: 1.5 mL/min; Run time: 6 min. Mobile phase: Initial 95% water (+10 mM ammonium bicarbonate) and 5% acetonitrile to 5% water (+10 mM ammonium bicarbonate) and 95% acetonitrile over 3 min then hold at 95% acetonitrile and 5% water (+10 mM ammonium bicarbonate) for 3 min.

LC Method CC: Analytical GC using a Phenomenex ZB-1 MS column (0.25×30 mm, 0.25 μm particle size); start temp 50° C., ramp 20° C./min to 300° C. and hold for 5 min.

LC Method DD: Analytical reverse phase HPLC-MS using a Merckmillipore Chromolith SpeedROD C₁₈ column (50×4.6 mm) and a dual gradient run from 5% to 100% mobile phase B over 12 minutes. Mobile phase A=water (+0.1% trifluoroacetic acid). Mobile phase B=acetonitrile (+0.1% trifluoroacetic acid).

LC Method EE: Analytical reverse phase HPLC-MS using a Kinetex EVO C₁₈ column (4.6×50 mm, 2.6 μm particle size), Temp: 45° C., Flow: 2.0 ml/min, Run Time: 3 minutes. Mobile Phase Conditions: Initial 95% water (+0.1% formic acid) and 5% acetonitrile (+0.1% formic acid) linear gradient to 95% acetonitrile (+0.1% formic acid) for 2.0 min then hold at 95% acetonitrile (+0.1% formic acid) for 1.0 min.

LC Method FF: Analytical reverse phase UPLC using an Acquity UPLC-MS BEH C₁₈ column (50×2.1 mm, 1.7 m particle) made by Waters (pn: 186002350), and a dual gradient run from 1% to 99% mobile phase B over 4.5 minutes (Mobile phase A=water (+0.05% CF₃CO₂H), Mobile phase B=acetonitrile (0.035% CF₃CO₂H), Flow rate=1.2 mL/min, injection volume=1.5 μL, and column temperature=60° C.).

LC Method GG: Analytical reverse phase UPLC-MS using an Acquity UPLC-MS BEH C₁₈ column (30×2.1 mm, 1.7 m particle size) made by Waters (pn: 186002349), and a dual gradient run from 50% to 99% mobile phase B over 1.0 minutes. Mobile phase A=water (+0.05% formic acid). Mobile phase B=acetonitrile (+0.035% formic acid). Flow rate=1.5 mL/min, injection volume=1.5 μL, and column temperature=60° C.

LC Method HH: Analytical reverse phase UPLC-MS using an Acquity UPLC-MS BEH C₁₈ column (30×2.1 mm, 1.7 m particle size) made by Waters (pn: 186002349), and a dual gradient run from 1% to 99% mobile phase B over 1.0 minutes. Mobile phase A=water (+0.05% formic acid). Mobile phase B=acetonitrile (+0.035% formic acid). Flow rate=1.5 mL/min, injection volume=1.5 μL, and column temperature=60° C.

LC Method II: Analytical reverse phase UPLC-MS using an Acquity UPLC-MS BEH C₁₈ column (50×2.1 mm, 1.7 m particle size) made by Waters (pn: 186002350), and a dual gradient run from 30% to 99% mobile phase B over 2.9 minutes. Mobile phase A=water (+0.05% formic acid). Mobile phase B=acetonitrile (+0.035% formic acid). Flow rate=1.2 mL/min, injection volume=1.5 μL, and column temperature=60° C.

LC Method JJ: Analytical reverse phase UPLC-MS using an Acquity UPLC-MS BEH C₁₈ column (50×2.1 mm, 1.7 m particle size) made by Waters (pn: 186002350), and a dual gradient run from 1% to 99% mobile phase B over 2.9 minutes. Mobile phase A=water (+0.05% formic acid). Mobile phase B=acetonitrile (+0.035% formic acid). Flow rate=1.2 mL/min, injection volume=1.5 μL, and column temperature=60° C.

### PREPARATION OF COMMON INTERMEDIATES

### Intermediate 1: Preparation of methyl 3-[bis(tert-butoxycarbonyl)amino]-6-bromo-5-(trifluoromethyl)pyridine-2-carboxylate

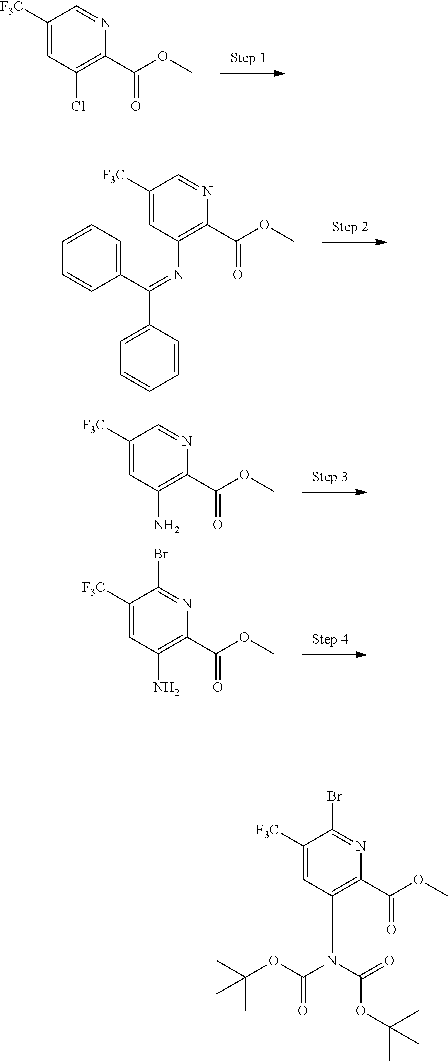

### Step 1: Methyl 3-(benzhydrylideneamino)-5-(trifluoromethyl)pyridine-2-carboxylate

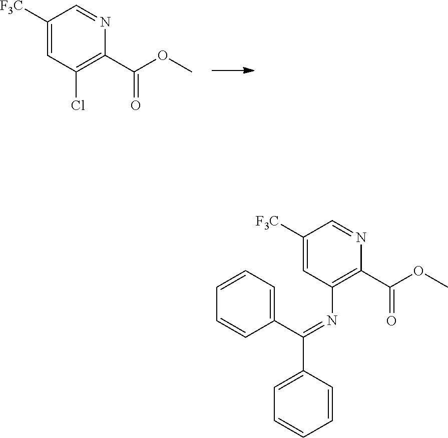 [SMILES: COC(=O)c1ncc(C(F)(F)F)cc1Cl ; COC(=O)c1ncc(C(F)(F)F)cc1N=C(c1ccccc1)c1ccccc1]

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed by bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with ethyl acetate and solvents were evaporated under reduced pressure to give methyl 3-(benzhydrylideneamino)-5-(trifluoromethyl)pyridine-2-carboxylate (90 g, 84%) as yellow solid. ESI-MS m/z calc. 384.10855, found 385.1 (M+1)⁺; Retention time: 2.24 minutes (LC Method B).

### Step 2: Methyl 3-amino-5-(trifluoromethyl)pyridine-2-carboxylate

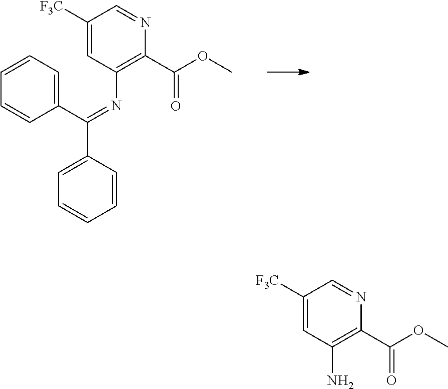 [SMILES: COC(=O)c1ncc(C(F)(F)F)cc1N=C(c1ccccc1)c1ccccc1 ; COC(=O)c1ncc(C(F)(F)F)cc1N]

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H). ¹⁹F NMR (282 MHz, CDCl₃) δ−63.23 (s, 3F) ppm. ESI-MS m/z calc. 220.046, found 221.1 (M+1)⁺; Retention time: 1.62 minutes (LC Method E).

### Step 3: Methyl 3-amino-6-bromo-5-(trifluoromethyl)pyridine-2-carboxylate

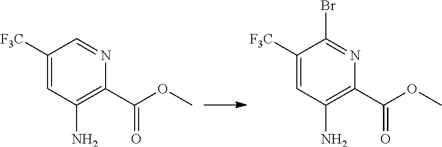 [SMILES: COC(=O)c1ncc(C(F)(F)F)cc1N ; COC(=O)c1nc(Br)c(C(F)(F)F)cc1N]

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) ppm 7.37 (s, 1H), 6.01 (br. s., 2H), 3.93-4.03 (m, 3H) ppm. ¹⁹F NMR (282 MHz, CDCl₃) ppm −64.2 (s, 3F) ppm. ESI-MS m/z calc. 297.9565, found 299.0 (M+1)⁺; Retention time: 2.55 minutes (LC Method F).

### Step 4: Methyl 3-[bis(tert-butoxycarbonyl)amino]-6-bromo-5-(trifluoromethyl)pyridine-2-carboxylate

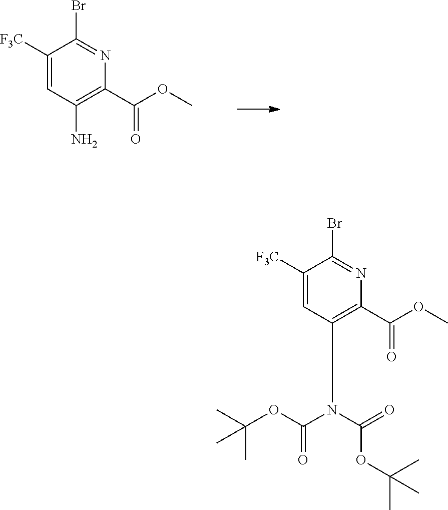 [SMILES: COC(=O)c1nc(Br)c(C(F)(F)F)cc1N ; COC(=O)c1nc(Br)c(C(F)(F)F)cc1N(C(=O)OC(C)(C)C)C(=O)OC(C)(C)C]

A mixture of methyl 3-amino-6-bromo-5-(trifluoromethyl)pyridine-2-carboxylate (5 g, 15.549 mmol), (Boc)₂O (11 g, 11.579 mL, 50.402 mmol), DMAP (310 mg, 2.5375 mmol) and CH₂Cl₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as a light yellow solid. ¹H NMR (300 MHz, CDCl₃) ppm 7.85 (s, 1H), 3.96 (s, 3H), 1.42 (s, 18H) ppm. ¹⁹F NMR (282 MHz, CDCl₃) ppm −63.9 (s, 3F) ppm. ESI-MS m/z calc. 498.06134, Retention time: 2.34 minutes (LC Method B).

### Intermediate 2: Preparation of 6-bromo-3-(tert-butoxycarbonylamino)-5-(trifluoromethyl)pyridine-2-carboxylic Acid

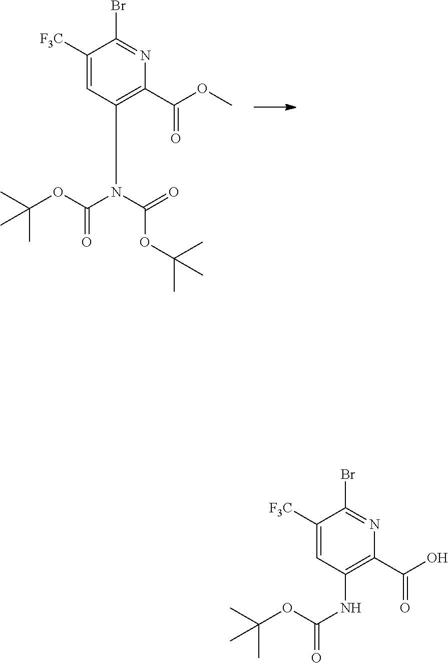 [SMILES: COC(=O)c1nc(Br)c(C(F)(F)F)cc1N(C(=O)OC(C)(C)C)C(=O)OC(C)(C)C ; CC(C)(C)OC(=O)Nc1cc(C(F)(F)F)c(Br)nc1C(=O)O]

To a mixture of methyl 3-[bis(tert-butoxycarbonyl)amino]-6-bromo-5-(trifluoromethyl)pyridine-2-carboxylate (247 g, 494.7 mmol) in THF (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2 mol) keeping the reaction temperature <15° C. The mixture was diluted with heptane (1.5 L), mixed and the organic phase separated. The aqueous phase was extracted with heptane (500 mL). The combined organic phases were washed with brine, dried over MgSO₄, filtered and concentrated. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d₆) δ 10.38 (s, 1H), 9.01 (s, 1H), 1.50 (s, 9H) ppm. ESI-MS m/z calc. 383.99326, found 330.9 (M+1)⁺; Retention time: 2.55 minutes (LC Method FF).

### Intermediate 3: Preparation of 2-Benzyloxy-2-(trifluoromethyl)hex-5-enehydrazide (Hydrochloride Salt)

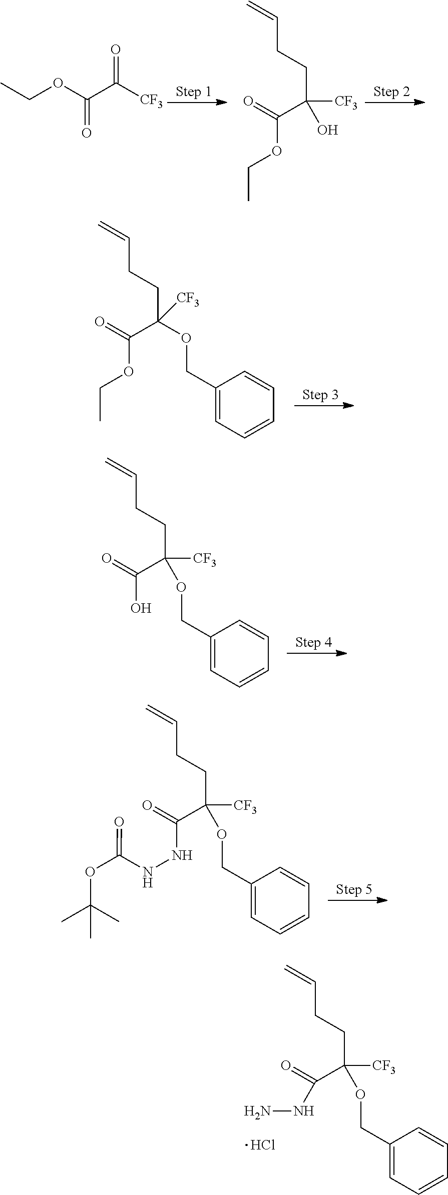

### Step 1: Ethyl 2-hydroxy-2-(trifluoromethyl)hex-5-enoate

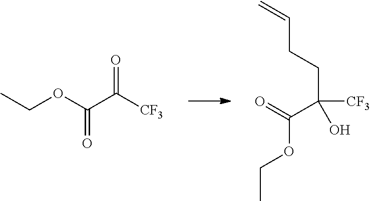

To a solution of ethyl 3,3,3-trifluoro-2-oxo-propanoate (25.15 g, 147.87 mmol) in Et₂O (270 mL) at −78° C. was added bromo(but-3-enyl)magnesium in THF (190 mL of 0.817 M, 155.23 mmol) dropwise over a period of 1.5 h (inner temperature −72° C. to −76° C.). The mixture was stirred at −78° C. for 20 min. The dry ice-acetone bath was removed. The mixture was slowly warm to 5° C. during 1 h, added to a mixture of 1N aqueous HCl (170 mL) and crushed ice (150 g) (pH=4). The two layers were separated. The organic layer was concentrated, and the residue was combined with aqueous phase and extracted with ethyl acetate (2×150 mL). The combined organic phase was washed with 5% aqueous NaHCO₃ (50 mL) and brine (20 mL), dried with Na₂SO₄. The mixture was filtered and concentrated and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (m, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ−78.74 (s, 3F) ppm.

### Step 2: Ethyl 2-benzyloxy-2-(trifluoromethyl)hex-5-enoate

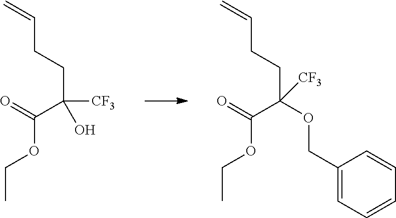 [SMILES: C=CCCC(O)(C(=O)OCC)C(F)(F)F ; C=CCCC(OCc1ccccc1)(C(=O)OCC)C(F)(F)F]

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and tetrabutylammonium iodide (8.68 g, 23.500 mmol) were added. The mixture was stirred at room temperature overnight. NH₄Cl (3 g, 0.6 equivalents) was added. The mixture was stirred for 10 min. 30 mL of ethyl acetate was added, then ice-water (400 g). The mixture was extracted with CH₂Cl₂ and the combined organic layer was concentrated. Purification by silica gel chromatography (0-20% CH₂Cl₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. ¹⁹F NMR (282 MHz, CDCl₃) δ−70.5 (s, 3F) ppm. ESI-MS m/z calc. 316.12863, found 317.1 (M+1)⁺; Retention time: 2.47 minutes (LC Method B).

### Step 3: 2-Benzyloxy-2-(trifluoromethyl)hex-5-enoic Acid

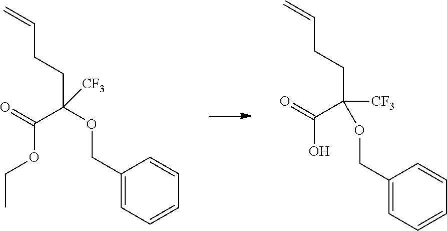 [SMILES: C=CCCC(OCc1ccccc1)(C(=O)OCC)C(F)(F)F ; C=CCCC(OCc1ccccc1)(C(=O)O)C(F)(F)F]

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as a pale yellow oil. ¹H NMR (300 MHz, DMSO-d₆) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d₆) δ−70.29 (s, 3F) ppm. ESI-MS m/z calc. 288.09732, found 287.1 (M−1); Retention time: 3.1 minutes (LC Method C).

### Step 4: tert-Butyl N-[[2-benzyloxy-2-(trifluoromethyl)hex-5-enoyl]amino]carbamate

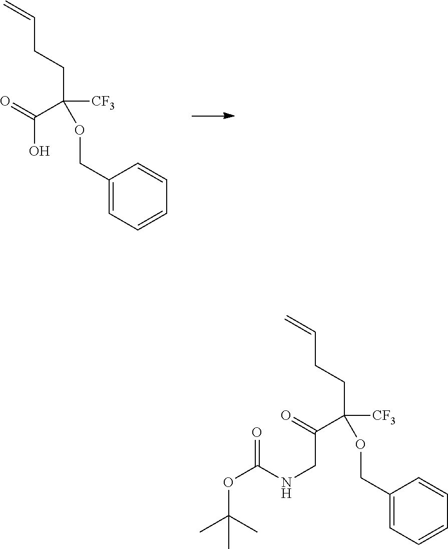 [SMILES: C=CCCC(OCc1ccccc1)(C(=O)O)C(F)(F)F ; C=CCCC(OCc1ccccc1)(C(=O)CNC(=O)OC(C)(C)C)C(F)(F)F]

To a solution of 2-benzyloxy-2-(trifluoromethyl)hex-5-enoic acid (21.92 g, 92.4% purity, 70.263 mmol) in DMF (130 mL) was added HATU (37.2 g, 97.836 mmol) and Et₃N (15 g, 148.24 mmol). The mixture was stirred for 10 minutes then tert-butyl N-aminocarbamate (12.2 g, 92.312 mmol) was added. The mixture was stirred at 25° C. overnight and at 40° C. for 1 h. The mixture was diluted with ice-water (500 g) and extracted with CH₂Cl₂. The organic layer dried over anhydrous sodium sulfate and was concentrated. Purification by silica gel chromatography (0-30% ethyl acetate in heptanes) provided tert-butyl N-[[2-benzyloxy-2-(trifluoromethyl)hex-5-enoyl]amino]carbamate (26.08 g, 92%) as white solid. ¹H NMR (300 MHz, CDCl₃) δ 1.46 (s, 9H), 2.10-2.31 (m, 3H), 2.34-2.51 (m, 1H), 4.60-4.72 (m, 1H), 4.73-4.86 (m, 1H), 4.95-5.19 (m, 2H), 5.83 (m, 1H), 6.28 (br. s., 1H), 7.30-7.51 (m, 5H), 8.34 (d, J=2.6 Hz, 1H) ppm. ¹⁹F NMR (282 MHz, CDCl₃) δ−73.6 (s, 3F) ppm.

### Step 5: 2-Benzyloxy-2-(trifluoromethyl)hex-5-enehydrazide (Hydrochloride Salt)

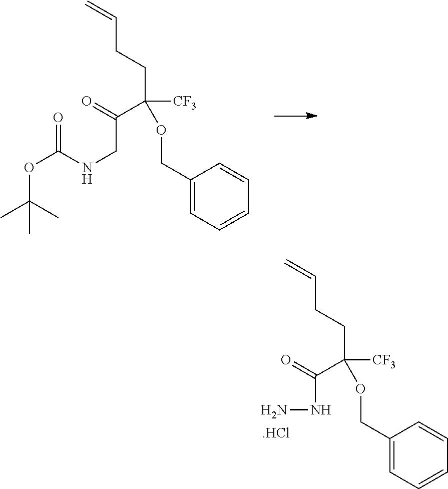 [SMILES: C=CCCC(OCc1ccccc1)(C(=O)CNC(=O)OC(C)(C)C)C(F)(F)F ; ".HCl" ; C=CCCC(OCc1ccccc1)(C(=O)NN)C(F)(F)F]

To a solution of tert-butyl N-[[2-benzyloxy-2-(trifluoromethyl)hex-5-enoyl]amino]carbamate (43.12 g, 107.2 mmol) in CH₂Cl₂ (200 mL) was added HCl (100 mL of 4 M, 400 mmol) and the mixture was stirred at ambient temperature for 7 h. The solvent was removed in vacuo, the residue stripped 2 times from heptane and the resultant solid was dried in vacuo using a high vac for 20 h giving 2-benzyloxy-2-(trifluoromethyl)hex-5-enehydrazide (hydrochloride salt) (35 g, 96%). ¹H NMR (400 MHz, Chloroform-d) δ 9.92 (s, 2H), 7.41-7.31 (m, 2H), 7.30-7.24 (m, 2H), 7.24-7.16 (m, 1H), 5.72-5.57 (m, 1H), 5.02-4.87 (m, 2H), 4.71 (d, J=10.9 Hz, 1H), 4.62 (d, J=11.0 Hz, 1H), 3.70 (s, 2H), 2.34-1.85 (m, 4H) ppm. ESI-MS m/z calc. 302.1242, found 303.2 (M+1)⁺; Retention time: 1.5 minutes (LC Method A).

### Intermediate 4: Preparation of tert-butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate

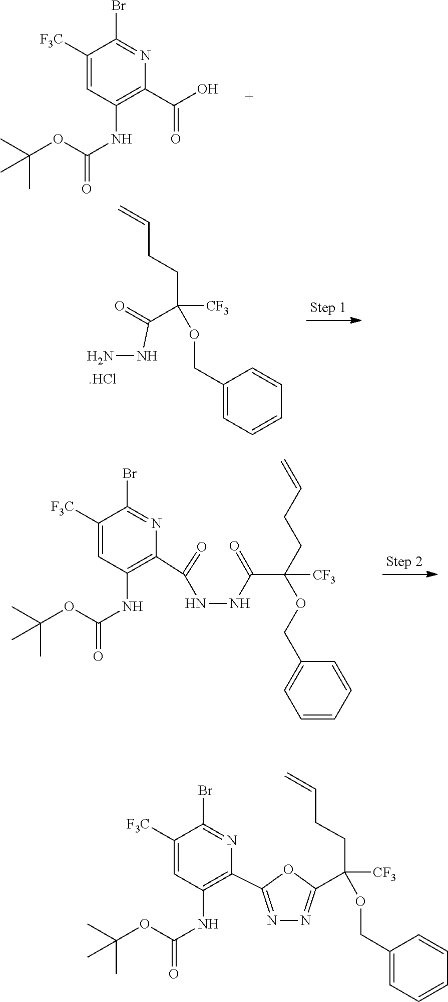

### Step 1: tert-Butyl N-[2-[[[2-benzyloxy-2-(trifluoromethyl)hex-5-enoyl]amino]carbamoyl]-6-bromo-5-(trifluoromethyl)-3-pyridyl]carbamate

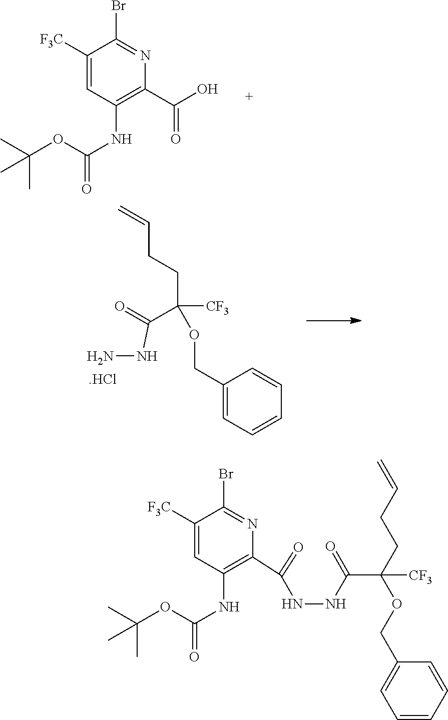

To a mixture of 6-bromo-3-(tert-butoxycarbonylamino)-5-(trifluoromethyl)pyridine-2-carboxylic acid (239.2 g, 621.1 mmol) and 2-benzyloxy-2-(trifluoromethyl)hex-5-enehydrazide (hydrochloride salt) (230.1 g, 761.2 mmol) in ethyl acetate (2.2 L) at ambient temperature was added pyridine (200 mL, 2.473 mol) which afforded a precipitate. To the mixture was added 1-propanephosphonic anhydride (500 g of 50% w/w, 785.7 mmol) and the reaction mixture was stirred at ambient temperature for 12 h. The reaction was quenched with the slow addition of NaOH (149 g of 50% w/w, 1.863 mol) in water (2 L) and the mixture was stirred for 15 min. The organic phase was separated, and the aqueous phase extracted with ethyl acetate (1 L). The combined organic phases washed with brine, dried over MgSO₄, filtered and concentrated in vacuo. After half of the solvent was removed, the organic phase was washed 2 times with aqueous HCl (1000 mL of 1 M, 1 mol). The organic phase was dried over MgSO₄, filtered and concentrated in vacuo. The crude product was slurried in warm heptane (2.5 L) and MTBE (0.25 L) and the mixture stirred at ambient temperature for 12 h affording a light yellow slurry. The slurry was filtered, and the resultant filter cake was washed 2 times with 1 L 10% MTBE/heptane. The off-white solid was air dried for 2 h, then in vacuo at 40° C. for 20 h giving tert-butyl N-[2-[[[2-benzyloxy-2-(trifluoromethyl)hex-5-enoyl]amino]carbamoyl]-6-bromo-5-(trifluoromethyl)-3-pyridyl]carbamate (379.9 g, 91%). ¹H NMR (400 MHz, DMSO-d₆) δ 11.09 (s, 1H), 10.92 (s, 1H), 10.35 (s, 1H), 9.15 (s, 1H), 7.50 (d, J=7.4 Hz, 2H), 7.36 (m, 3H), 5.87 (m, 1H), 5.09 (d, J=16.9 Hz, 1H), 5.02 (d, J=10.1 Hz, 1H), 4.84 (q, J=11.4 Hz, 2H), 2.35-2.12 (m, 4H), 1.49 (s, 9H) ppm. ESI-MS m/z calc. 668.1069, found 670.9 (M+1)⁺; Retention time: 3.5 minutes (LC Method D).

### Step 2: tert-Butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate

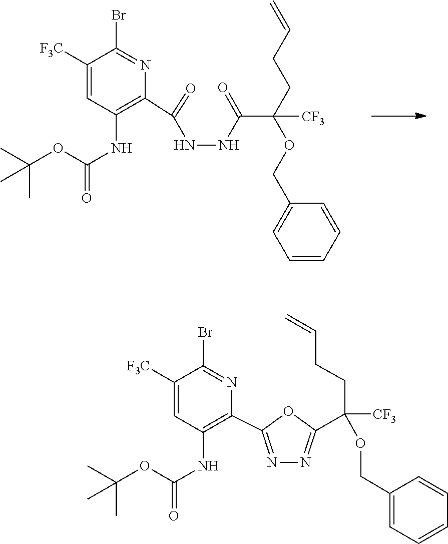 [SMILES: C=CCCC(OCc1ccccc1)(C(=O)NNC(=O)c1nc(Br)c(C(F)(F)F)cc1NC(=O)OC(C)(C)C)C(F)(F)F ; C=CCCC(OCc1ccccc1)(c1nnc(-c2nc(Br)c(C(F)(F)F)cc2NC(=O)OC(C)(C)C)o1)C(F)(F)F]

tert-Butyl N-[2-[[[2-benzyloxy-2-(trifluoromethyl)hex-5-enoyl]amino]carbamoyl]-6-bromo-5-(trifluoromethyl)-3-pyridyl]carbamate (102 g, 150.8 mmol) was dissolved in anhydrous acetonitrile (1000 mL) and DIEA (92 mL, 528.2 mmol) was added. The resultant orange solution was heated to 70° C. (internal temp) making a clear yellow solution. Then p-toluenesulfonyl chloride (37.4 g, 196.2 mmol) was added in 3 equal portions of 12.47 g separated by 10 minutes and then the reaction was heated for another 30 min. The reaction was cooled to room temperature and the acetonitrile was concentrated under reduced pressure. To the mixture was added 1000 mL MTBE, then 800 mL water, and the mixture was stirred, and the layers were separated. The organic layer was washed with a solution of citric acid (36.3 g, 188.9 mmol) in 700 mL water, then 400 mL saturated NaHCO₃, then 300 mL brine. The organic layer was then dried over anhydrous MgSO₄, filtered and concentrated under reduced pressure. The material was purified by silica gel chromatography using a gradient of 15% to 50% of an 8% ethyl acetate in hexanes to hexanes gradient to provide tert-butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate (91.7 g, 93%). ¹H NMR (400 MHz, Chloroform-d) δ 10.18 (s, 1H), 9.35 (s, 1H), 7.55-7.47 (m, 2H), 7.45-7.37 (m, 2H), 7.36-7.28 (m, 1H), 5.83-5.68 (m, 1H), 5.10-4.93 (m, 2H), 4.82 (d, J=10.5 Hz, 1H), 4.69 (d, J=10.5 Hz, 1H), 2.59-2.13 (m, 4H), 1.56 (s, 9H) ppm. ESI-MS m/z calc. 650.0963, found 651.0 (M+1)⁺; Retention time: 3.81 minutes (LC Method D).

### Intermediate 5: Preparation of (2R)-2-benzyloxy-2-(trifluoromethyl)hex-5-enoic Acid

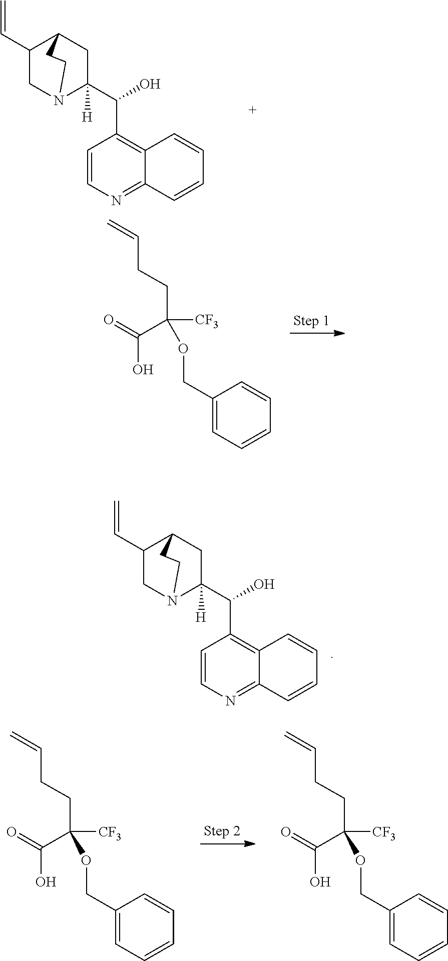

### Step-1: (2R)-2-Benzyloxy-2-(trifluoromethyl)hex-5-enoic acid; (R)-4-quinolyl-[(2S,4S)-5-vinylquinuclidin-2-yl]methanol

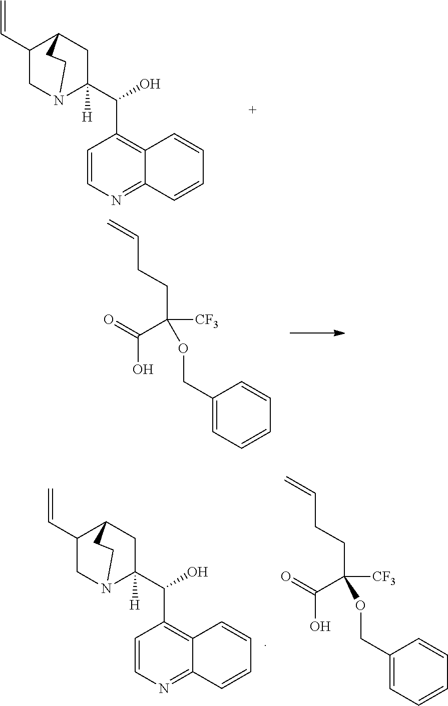

To a N2 purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid; (R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20 minutes (Mobile phase A=H₂O (0.1% CF₃CO₂H), mobile phase B=MeOH (0.1% CF₃CO₂H), flow rate=1.0 mL/min, injection volume=2 μL, and column temperature=30° C., sample concentration: 1 mg/mL in 60% acetonitrile/40% water).

### Step 2: (2R)-2-Benzyloxy-2-(trifluoromethyl)hex-5-enoic Acid

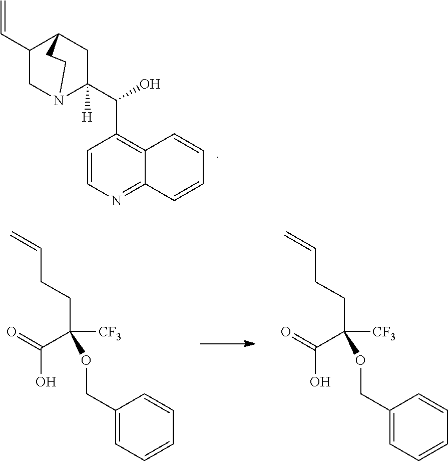 [SMILES: C=CC1CN2CC[C@H]1C[C@H]2[C@H](O)c1ccnc2ccccc12 ; C=CCC[C@@](OCc1ccccc1)(C(=O)O)C(F)(F)F ; C=CCC[C@@](OCc1ccccc1)(C(=O)O)C(F)(F)F]

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500 mL) was treated with an aqueous solution of hydrochloric acid (200 mL of 1 M, 200 mmol). After stirring 15 minutes at room temperature, the two phases were separated. The aqueous phase was extracted twice with ethyl acetate (200 mL). The combined organic layer was washed with 1N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ−71.63 (br s, 3F) ppm. ESI-MS m/z calc. 288.0973, found 287.0 (M−1); Retention time: 2.15 minutes (LC Method B).

### Intermediate 6: Preparation of (2R)-2-benzyloxy-2-(trifluoromethyl)hex-5-enehydrazide

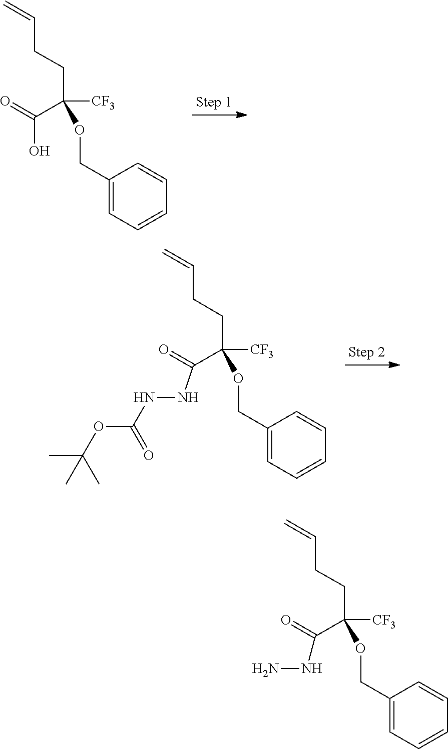

### Step 1: tert-Butyl N-[[(2R)-2-benzyloxy-2-(trifluoromethyl)hex-5-enoyl]amino]carbamate

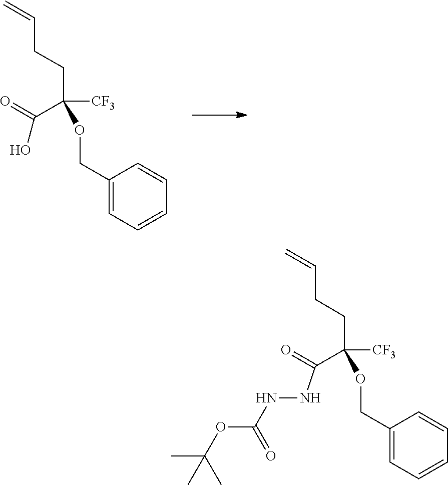 [SMILES: C=CCC[C@@](OCc1ccccc1)(C(=O)O)C(F)(F)F ; C=CCC[C@@](OCc1ccccc1)(C(=O)NNC(=O)OC(C)(C)C)C(F)(F)F]

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in ethyl acetate (2 L) and washed with brine. The organic phase was dried over MgSO₄, filtered and concentrated in vacuo. The oil was diluted with ethyl acetate (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% ethyl acetate/hexanes) provided tert-butyl N-[[(2R)-2-benzyloxy-2-(trifluoromethyl)hex-5-enoyl]amino]carbamate (464 g, 91%, combined with product from crystallization). ESI-MS m/z calc. 402.17664, found 303.0 (M+1-Boc)*; Retention time: 2.68 minutes (LC Method D).

### Step 2: (2R)-2-Benzyloxy-2-(trifluoromethyl)hex-5-enehydrazide

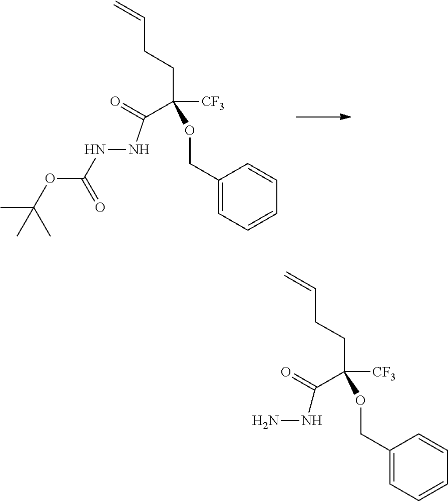 [SMILES: C=CCC[C@@](OCc1ccccc1)(C(=O)NNC(=O)OC(C)(C)C)C(F)(F)F ; C=CCC[C@@](OCc1ccccc1)(C(=O)NN)C(F)(F)F]

To a solution of tert-butyl N-[[(2R)-2-benzyloxy-2-(trifluoromethyl)hex-5-enoyl]amino]carbamate (464 g, 1.153 mol) in dichloromethane (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with 1 L of brine and the combined aqueous phases were extracted with isopropyl acetate (1 L). The combined organic phases were dried over MgSO₄, filtered and concentrated in vacuo affording a dark yellow oil. (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (m, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (m, J=14.3, 11.0, 5.9 Hz, 1H), 2.26-1.95 (m, 3H) ppm. ESI-MS m/z calc. 302.1242, found 303.0 (M+1)⁺; Retention time: 2.0 minutes (LC Method D).

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

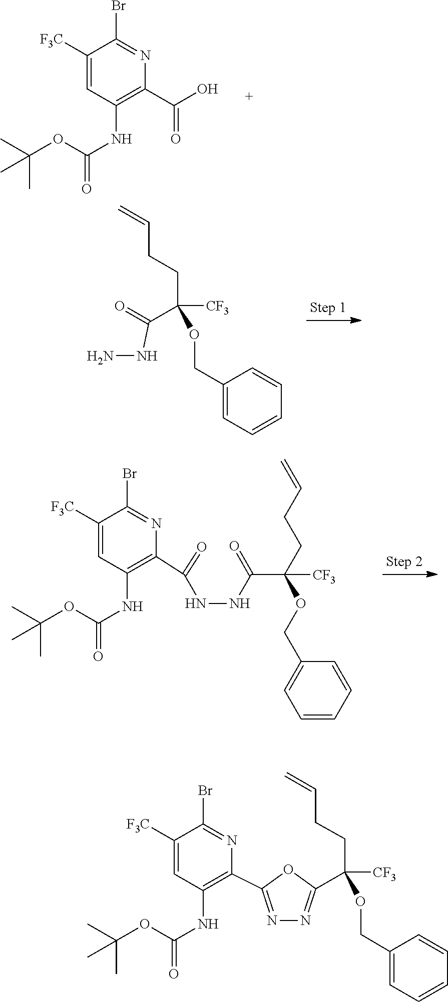

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

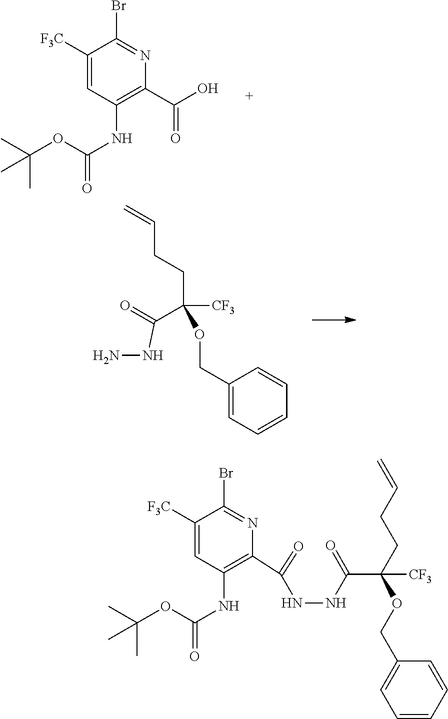

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in ethyl acetate (2.25 L) at ambient temperature was added DIEA (425 mL, 2.440 mol). To the mixture was slowly added T3P (622 g of 50% w/w, 977.4 mmol) using an ice-water bath to keep the temperature <35° C. (temperature rose to 34° C.) and the reaction mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with ethyl acetate (2 L). The combined organic phases were washed with brine, dried over MgSO₄, filtered and concentrated in vacuo. The crude product was dissolved in MTBE (300 mL) and diluted with heptane (3 L); the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% ethyl acetate/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (m, J=17.0, 10.2, 5.1 Hz, 1H), 5.09 (m, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m/z calc. 668.1069, found 669.0 (M+1)⁺; Retention time: 3.55 minutes (LC Method FF).

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

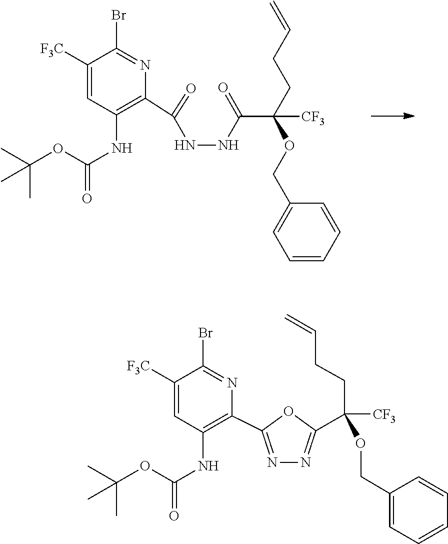 [SMILES: C=CCC[C@@](OCc1ccccc1)(C(=O)NNC(=O)c1nc(Br)c(C(F)(F)F)cc1NC(=O)OC(C)(C)C)C(F)(F)F ; C=CCC[C@@](OCc1ccccc1)(c1nnc(-c2nc(Br)c(C(F)(F)F)cc2NC(=O)OC(C)(C)C)o1)C(F)(F)F]

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with ethyl acetate (1.5 L) and water (1.5 L). The organic phase was separated and washed with 500 mL of 1 M HCl, 500 mL of brine, dried over MgSO₄, filtered and concentrated in vacuo. Purification by silica gel chromatography (0-20% ethyl acetate/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (m, J=16.7, 10.2, 6.4 Hz, 1H), 5.11 (m, J=17.2, 1.7 Hz, 1H), 5.01 (m, J=10.2, 1.5 Hz, 1H), 4.74 (d, J=10.6 Hz, 1H), 4.65 (d, J=10.6 Hz, 1H), 2.55-2.42 (m, 2H), 2.30 (m, J=11.3, 10.3, 6.9 Hz, 2H), 1.52 (s, 9H) ppm. ESI-MS m/z calc. 650.0963, found 650.0 (M+1)⁺; Retention time: 3.78 minutes (LC Method FF).

### Intermediate 8: Preparation of methyl 3-[bis(tert-butoxycarbonyl)amino]-6-pent-4-enoyl-5-(trifluoromethyl)pyridine-2-carboxylate

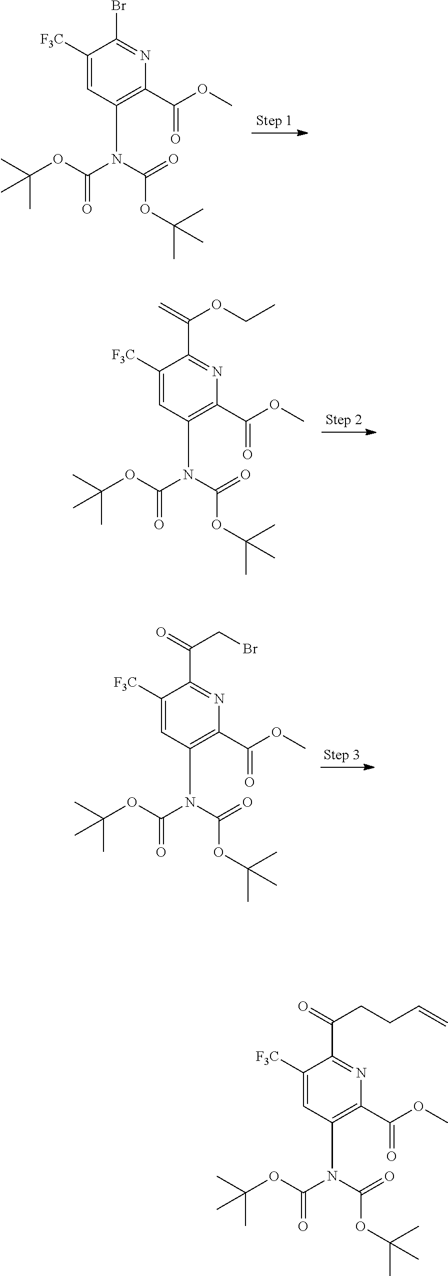 [SMILES: COC(=O)c1nc(Br)c(C(F)(F)F)cc1N(C(=O)OC(C)(C)C)C(=O)OC(C)(C)C ; C=C(OCC)c1nc(C(=O)OC)c(N(C(=O)OC(C)(C)C)C(=O)OC(C)(C)C)cc1C(F)(F)F ; COC(=O)c1nc(C(=O)CBr)c(C(F)(F)F)cc1N(C(=O)OC(C)(C)C)C(=O)OC(C)(C)C ; C=CCCC(=O)c1nc(C(=O)OC)c(N(C(=O)OC(C)(C)C)C(=O)OC(C)(C)C)cc1C(F)(F)F]

### Step 1: Methyl 3-[bis(tert-butoxycarbonyl)amino]-6-(1-ethoxyvinyl)-5-(trifluoromethyl)pyridine-2-carboxylate

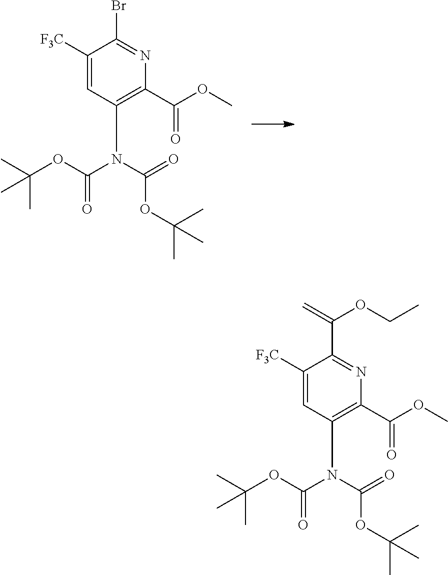 [SMILES: COC(=O)c1nc(Br)c(C(F)(F)F)cc1N(C(=O)OC(C)(C)C)C(=O)OC(C)(C)C ; C=C(OCC)c1nc(C(=O)OC)c(N(C(=O)OC(C)(C)C)C(=O)OC(C)(C)C)cc1C(F)(F)F]

To an oven dried 3 necked flask (1 L) equipped with magnetic stir bar was added methyl 3-[bis(tert-butoxycarbonyl)amino]-6-bromo-5-(trifluoromethyl)pyridine-2-carboxylate (10 g, 20.03 mmol) and PdCl₂(PPh₃)₂ (1.4 g, 1.995 mmol). The flask was evacuated and filled with nitrogen gas three times. Tributyl(1-ethoxyvinyl)stannane (7.6 g, 21.04 mmol) in dioxane (200 mL) was injected through a septum under an inert atmosphere. The mixture was stirred at 75° C. for 14 hours. The mixture was cooled to rt, filtered through a celite pad, washed with ethyl acetate and concentrated. Purification by silica gel chromatography (15:85 ethyl acetate:hexanes) provided methyl 3-[bis(tert-butoxycarbonyl)amino]-6-(1-ethoxyvinyl)-5-(trifluoromethyl)pyridine-2-carboxylate (8.6 g, 88%). ¹H NMR (400 MHz, Chloroform-d) δ 7.89 (s, 1H), 4.78 (d, J=2.7 Hz, 1H), 4.52 (d, J=2.8 Hz, 1H), 3.97-3.92 (m, 5H), 1.42-1.36 (m, 21H) ppm. ESI-MS m/z calc. 490.1927, found 491.2 (M+1)⁺; Retention time: 1.52 minutes (LC Method J).

### Step 2: Methyl 3-[bis(tert-butoxycarbonyl)amino]-6-(2-bromoacetyl)-5-(trifluoromethyl)pyridine-2-carboxylate

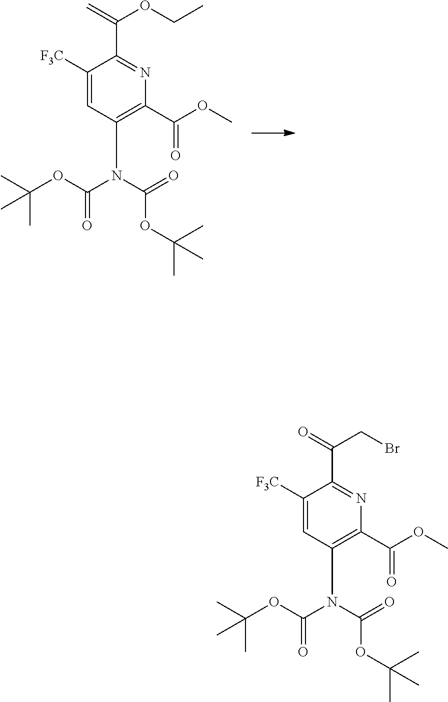 [SMILES: C=C(OCC)c1nc(C(=O)OC)c(N(C(=O)OC(C)(C)C)C(=O)OC(C)(C)C)cc1C(F)(F)F ; COC(=O)c1nc(C(=O)CBr)c(C(F)(F)F)cc1N(C(=O)OC(C)(C)C)C(=O)OC(C)(C)C]

1-Bromopyrrolidine-2,5-dione (3.4 g, 19.10 mmol) was added to a solution of methyl 3-[bis(tert-butoxycarbonyl)amino]-6-(1-ethoxyvinyl)-5-(trifluoromethyl)pyridine-2-carboxylate (8.5 g, 17.33 mmol) in THF (100 mL) and water (20 mL). The reaction mixture was stirred at room temperature for 2 hours. The reaction was quenched by a solution of NaHCO₃, extracted by dichloromethane (3×100 mL), washed with brine, organic layers were combined, dried over sodium sulfate and concentrated under vacuum. Purification by silica gel chromatography (15:85 ethyl acetate:hexanes) provided methyl 3-[bis(tert-butoxycarbonyl)amino]-6-(2-bromoacetyl)-5-(trifluoromethyl)pyridine-2-carboxylate (8.0 g, 85%). ¹H NMR (400 MHz, Chloroform-d) δ 8.04 (s, 1H), 4.74 (s, 2H), 3.99 (s, 3H), 1.43 (s, 18H) ppm. ESI-MS m/z calc. 540.0719, found 542.0 (M+1)⁺; Retention time: 0.7 minutes (LC Method M).

### Step 3: Methyl 3-[bis(tert-butoxycarbonyl)amino]-6-pent-4-enoyl-5-(trifluoromethyl)pyridine-2-carboxylate

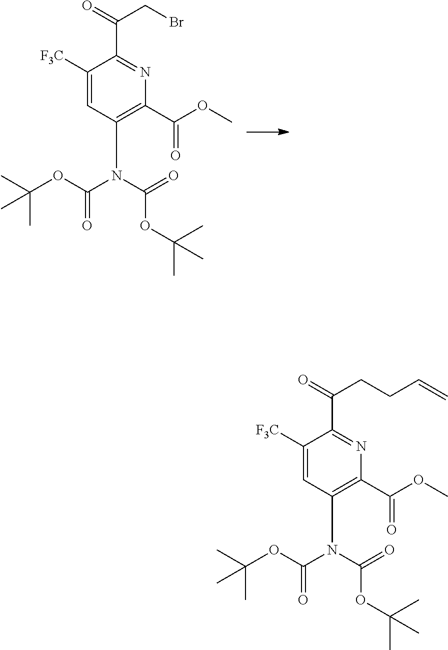 [SMILES: COC(=O)c1nc(C(=O)CBr)c(C(F)(F)F)cc1N(C(=O)OC(C)(C)C)C(=O)OC(C)(C)C ; C=CCCC(=O)c1nc(C(=O)OC)c(N(C(=O)OC(C)(C)C)C(=O)OC(C)(C)C)cc1C(F)(F)F]

To an oven dried 250 mL flask was added methyl 3-[bis(tert-butoxycarbonyl)amino]-6-(2-bromoacetyl)-5-(trifluoromethyl)pyridine-2-carboxylate (8 g, 14.78 mmol) and allyl(tributyl)stannane (5.2 g, 15.70 mmol) with benzene (100 mL). Then, 2-[(E)-(1-cyano-1-methyl-ethyl)azo]-2-methyl-propanenitrile (2.5 g, 15.22 mmol) was added and the flask evacuate and filled with nitrogen 3 times. The mixture was stirred at 70° C. for 15 h. The reaction mixture was cooled to room temperature and concentrated to dryness under reduced pressure. Purification by silica gel chromatography (0-15% ethyl acetate in hexanes) provided methyl 3-[bis(tert-butoxycarbonyl)amino]-6-pent-4-enoyl-5-(trifluoromethyl)pyridine-2-carboxylate (5.7 g, 77%). ¹H NMR (400 MHz, Chloroform-d) δ 7.98 (s, 1H), 5.89 (m, 1H), 5.10 (m, J=17.1, 1.6 Hz, 1H), 5.02 (m, J=10.2, 1.4 Hz, 1H), 3.98 (s, 3H), 3.26 (t, J=7.4 Hz, 2H), 2.60-2.44 (m, 2H), 1.43 (s, 18H) ppm. ESI-MS m/z calc. 502.1927, found 503.2 (M+1)⁺; Retention time: 1.58 minutes (LC Method J).

### Intermediate 9: Preparation of 3-(tert-butoxycarbonylamino)-6-pent-4-enoyl-5-(trifluoromethyl)pyridine-2-carboxylic Acid

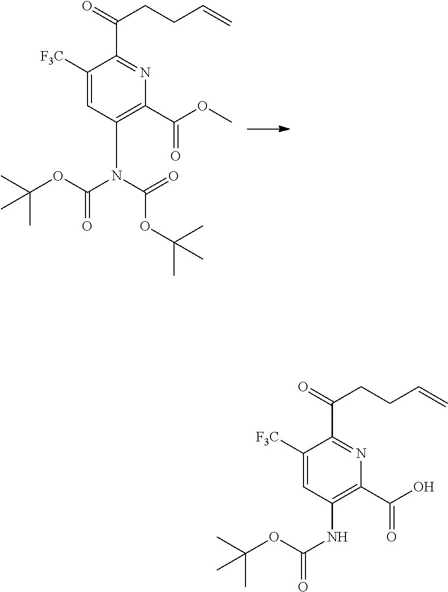 [SMILES: C=CCCC(=O)c1nc(C(=O)OC)c(N(C(=O)OC(C)(C)C)C(=O)OC(C)(C)C)cc1C(F)(F)F ; C=CCCC(=O)c1nc(C(=O)O)c(NC(=O)OC(C)(C)C)cc1C(F)(F)F]

To a solution of methyl 3-[bis(tert-butoxycarbonyl)amino]-6-pent-4-enoyl-5-(trifluoromethyl)pyridine-2-carboxylate (4.1 g, 8.160 mmol) in THF (50 mL) was added MeOH (40 mL), and water (30 mL). LiOH (587 mg, 24.51 mmol) was added to the mixture in three portions (Each time ˜196 mg with 5 min gap in between). The mixture was stirred at 60° C. for 1 h. THE and methanol were removed under reduced pressure and then 22 mL HCl (10%, ˜25 mmol) was added and the organic compound was extracted by ethyl acetate (3×100 mL). The organic phases were combined, washed with brine (50 mL), dried on anhydrous sodium sulfate, filtered and concentrated under reduced pressure. Purification by silica gel chromatography (0% to 50% ethyl acetate in hexanes) provided 3-(tert-butoxycarbonylamino)-6-pent-4-enoyl-5-(trifluoromethyl)pyridine-2-carboxylic acid (2.4 g, 76%). ¹H NMR (400 MHz, Chloroform-d) δ 10.86 (bs, 1H), 10.40 (s, 1H), 9.46 (s, 1H), 6.32-5.67 (m, 1H), 5.34-4.77 (m, 2H), 3.16 (t, J=7.3 Hz, 2H), 2.83-2.29 (m, 2H), 1.56 (s, 9H) ppm. ESI-MS m/z calc. 388.1246, found 389.0 (M+1)⁺; Retention time: 1.45 minutes (LC Method J).

### Intermediate 10: Preparation of methyl 3-(tert-butoxycarbonylamino)-6-pent-4-enoyl-5-(trifluoromethyl)pyridine-2-carboxylate

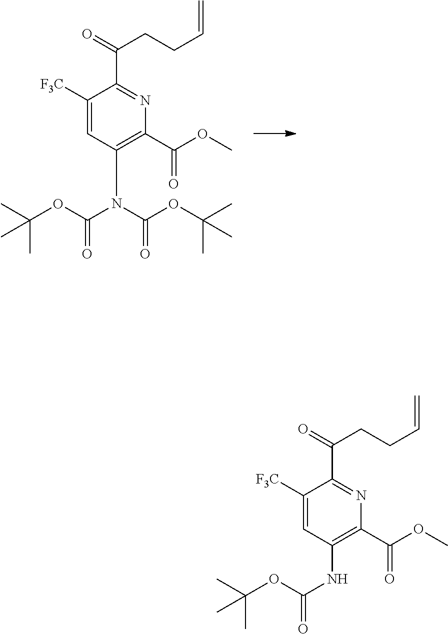 [SMILES: C=CCCC(=O)c1nc(C(=O)OC)c(N(C(=O)OC(C)(C)C)C(=O)OC(C)(C)C)cc1C(F)(F)F ; C=CCCC(=O)c1nc(C(=O)OC)c(NC(=O)OC(C)(C)C)cc1C(F)(F)F]

Methyl 3-[bis(tert-butoxycarbonyl)amino]-6-pent-4-enoyl-5-(trifluoromethyl)pyridine-2-carboxylate (2.21 g, 4.3982 mmol) dissolved in ethyl acetate (55 mL) and treated with silica gel (18.5 g, 307.90 mmol). The reaction was stirred at 80° C. overnight. The reaction was filtrated and concentrated to provide methyl 3-(tert-butoxycarbonylamino)-6-pent-4-enoyl-5-(trifluoromethyl)pyridine-2-carboxylate (1.98 g, quant.) as a yellow solid. ESI-MS m/z calc. 402.1403, found 403.2 (M+1)⁺; Retention time: 2.42 minutes (LC Method B).

### Intermediate 11: Preparation of 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic Acid

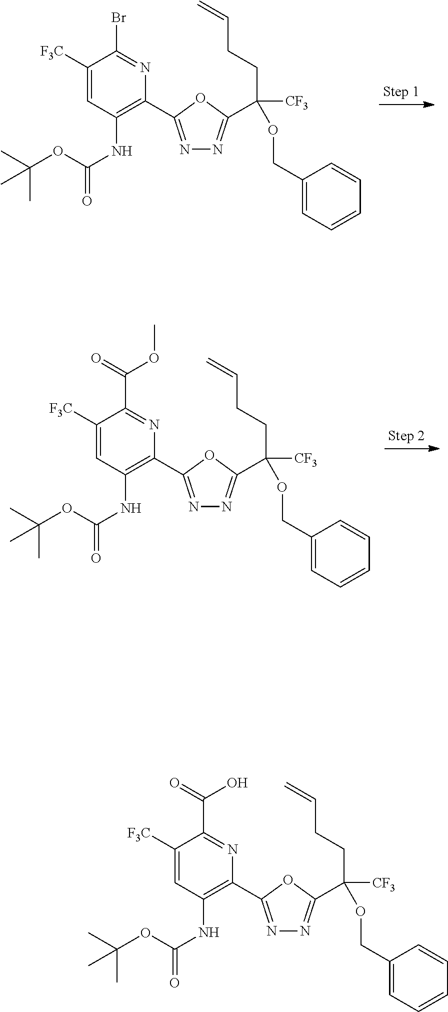 [SMILES: C=CCCC(OCc1ccccc1)(c1nnc(-c2nc(Br)c(C(F)(F)F)cc2NC(=O)OC(C)(C)C)o1)C(F)(F)F ; C=CCCC(OCc1ccccc1)(c1nnc(-c2nc(C(=O)OC)c(C(F)(F)F)cc2NC(=O)OC(C)(C)C)o1)C(F)(F)F ; C=CCCC(OCc1ccccc1)(c1nnc(-c2nc(C(=O)O)c(C(F)(F)F)cc2NC(=O)OC(C)(C)C)o1)C(F)(F)F]

### Step 1: Methyl 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylate

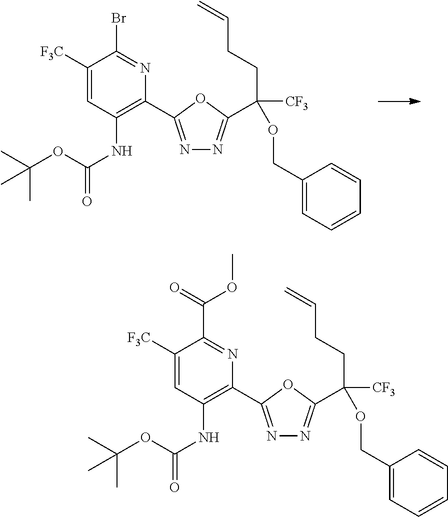 [SMILES: C=CCCC(OCc1ccccc1)(c1nnc(-c2nc(Br)c(C(F)(F)F)cc2NC(=O)OC(C)(C)C)o1)C(F)(F)F ; C=CCCC(OCc1ccccc1)(c1nnc(-c2nc(C(=O)OC)c(C(F)(F)F)cc2NC(=O)OC(C)(C)C)o1)C(F)(F)F]

Into a solution of tert-butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate (5.18 g, 7.9522 mmol) in MeOH (200 mL) was added TEA (2.5410 g, 3.5 mL, 25.111 mmol). The reaction was purged with nitrogen for 5 min and then, Pd(dppf)Cl₂ (604.1 mg, 0.8091 mmol) was added to the reaction mixture. The reaction was then stirred at 80° C. under 50 psi of carbon monoxide for 6 h. The reaction was cooled to rt, and then the MeOH was removed under vacuum. The residue was diluted with ethyl acetate (100 mL) and water (100 mL). Two layers were separated and the aqueous layer was extracted with ethyl acetate (2×100 mL). The combined organic layers were washed with saturated aqueous NaCl (100 mL), dried over anhydrous magnesium sulfate and concentrated under vacuum. Purification by silica gel chromatography (0 to 20% ethyl acetate/Hexanes) provided methyl 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylate (3.10 g, 62%) as a light yellow semi solid. ESI-MS m/z calc. 630.1913, found 631.5 (M+1)⁺; Retention time: 3.94 minutes (LC Method G).

### Step 2: 6-[5-[1-Benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic Acid

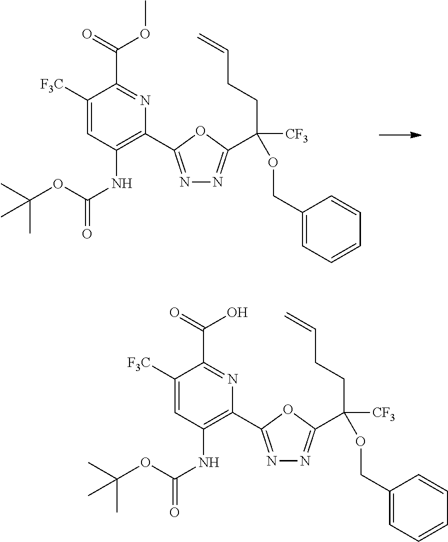 [SMILES: C=CCCC(OCc1ccccc1)(c1nnc(-c2nc(C(=O)OC)c(C(F)(F)F)cc2NC(=O)OC(C)(C)C)o1)C(F)(F)F ; C=CCCC(OCc1ccccc1)(c1nnc(-c2nc(C(=O)O)c(C(F)(F)F)cc2NC(=O)OC(C)(C)C)o1)C(F)(F)F]

Into a solution of methyl 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylate (3.10 g, 4.9165 mmol) in a solvent mixture of THF (38 mL) and DI Water (12 mL) was added LiOH (Water (1)) (1.06 g, 25.260 mmol). The reaction mixture turned yellow. The reaction was stirred at room temperature for 24 h. The reaction mixture was acidified to pH 5 using aqueous 1 M HCl and diluted with water (100 mL) and ethyl acetate (100 mL). Two layers were separated and the ethyl acetate layer was washed with DI H₂O (50 mL), dried over anhydrous sodium sulfate and concentrated under vacuum. Purification by silica gel chromatography (0% to 5% methanol in dichloromethane buffered with 0.3% acetic acid) provided 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic acid (1.22 g, 36%) as a white foamy solid. ¹H NMR (500 MHz, DMSO-d₆) δ 10.27 (s, 1H), 9.12 (s, 1H), 7.57-7.48 (m, 2H), 7.43-7.36 (m, 2H), 7.37-7.31 (m, 1H), 5.95-5.81 (m, 1H), 5.18-5.07 (m, 1H), 5.07-4.95 (m, 1H), 4.74-4.59 (m, 2H), 2.58-2.50 (m, 2H), 2.48-2.40 (m, 1H), 2.38-2.22 (m, 2H), 1.53 (s, 9H) ppm. ESI-MS m/z calc. 616.17566, found 617.2 (M+1)⁺; Retention time: 3.49 minutes (LC Method G).

### Intermediate 12: Preparation of 6-[5-[(1R)-1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic Acid

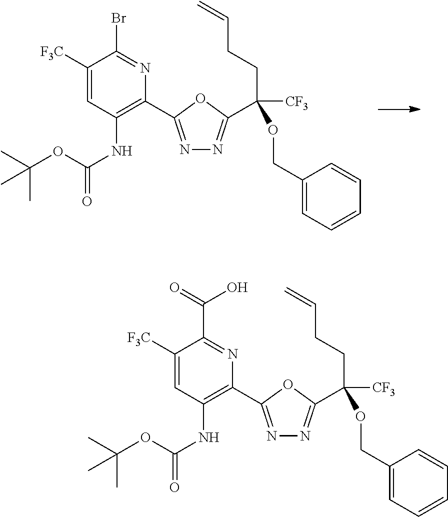 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(C(=O)O)c(C(F)(F)F)cc2NC(=O)OC(C)(C)C)o1)C(F)(F)F]

To tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (3.08 g, 4.728 mmol) in ether (45 mL) at −78° C. was added n-BuLi (4.2 mL of 2.5 M, 10.5 mmol) as a solution in hexane and the mixture stirred at −78° C. for 15 min then warmed to OC for 5 seconds and small pieces of solid CO₂ were added. The mixture was stirred at OC for 15 min. The mixture was diluted with citric acid (15 mL of 1 M, 15 mmol) to pH 4 and ethyl acetate was added. The organic layer was separated and washed with water, brine, dried (MgSO₄) and evaporated. Purification by silica gel chromatography (5-50% of the solution (10% MeOH in ethyl acetate) to hexanes) provided 6-[5-[(1R)-1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic acid (1.67 g, 53%). ¹H NMR (400 MHz, Chloroform-d) δ 10.45 (s, 1H), 9.61 (s, 1H), 7.48-7.28 (m, 5H), 5.78 (m, J=16.7, 10.2, 6.4 Hz, 1H), 5.07 (m, J=17.1, 1.6 Hz, 1H), 5.00 (d, J=1.5 Hz, 1H), 4.84 (d, J=11.0 Hz, 1H), 4.69 (d, J=11.0 Hz, 1H), 2.65-2.44 (m, 2H), 2.43-2.33 (m, 1H), 2.32-2.14 (m, 1H), 1.59 (s, 9H) ppm; ¹⁹F NMR (376 MHz, Chloroform-d) δ−61.10, −72.83 ppm. ESI-MS m/z calc. 616.17566, found 617.2 (M+1)⁺; Retention time: 0.42 minutes (LC Method T).

### Intermediate 13: Preparation of 2-benzyloxy-2-(trifluoromethyl)pent-4-enehydrazide (Hydrochloride Salt)

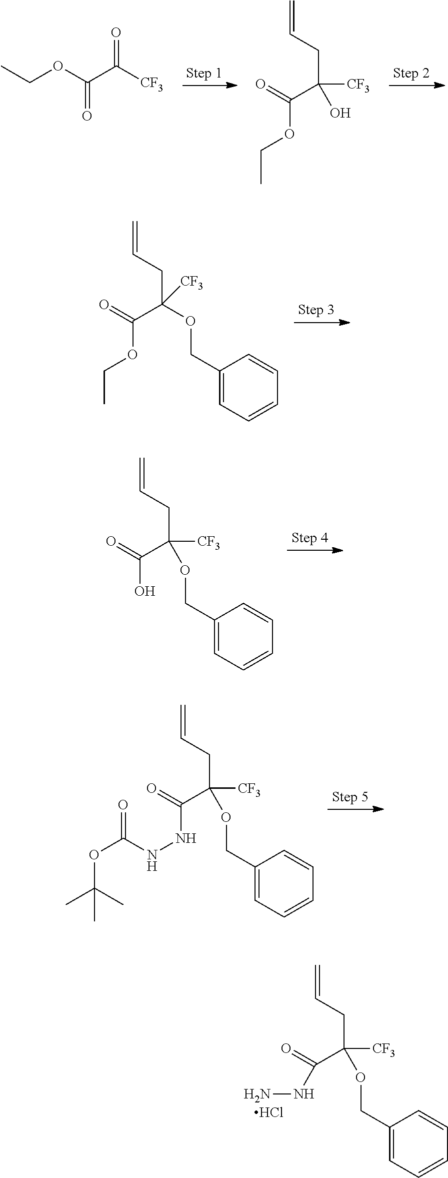

### Step 1: Ethyl 2-hydroxy-2-(trifluoromethyl)pent-4-enoate

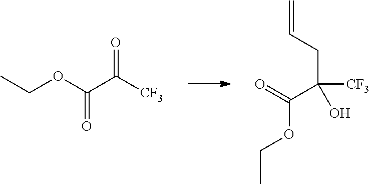

To a solution of ethyl 3,3,3-trifluoro-2-oxo-propanoate (30 g, 176.38 mmol) in diethyl ether (300 mL) at −78° C. was added allyl(bromo)magnesium (185 mL of 1 M, 185 mmol) dropwise over a period of 3 hours (internal temperature: −74° C.-−76° C.). The mixture was stirred at −78° C. for 45 min. The dry ice-acetone bath was removed. The mixture was allowed to warm to about 10° C. over a period of 1 h and added to a mixture of 1N aqueous HCl (210 mL) and crushed ice (400 g) (pH 4). The mixture was extracted with ethyl acetate, washed with 5% aqueous NaHCO₃, brine and dried over anhydrous Na₂SO₄. The mixture was filtered, concentrated and co-evaporated with hexane to give ethyl 2-hydroxy-2-(trifluoromethyl)pent-4-enoate (42.2 g, 90%) as light yellow oil. ¹H NMR (300 MHz, CDCl₃) δ 1.33 (t, J=7.1 Hz, 3H), 2.60-2.79 (m, 2H), 3.84 (br. s., 1H), 4.24-4.48 (m, 2H), 5.09-5.33 (m, 2H), 5.59-5.82 (m, 1H) ppm. ¹⁹F NMR (282 MHz, CDCl₃) δ−78.5 (s, 3F) ppm.

### Step 2: Ethyl 2-benzyloxy-2-(trifluoromethyl)pent-4-enoate

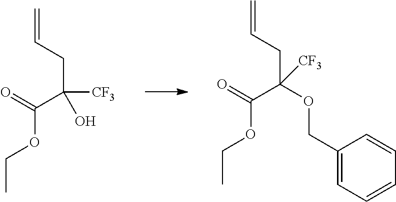 [SMILES: C=CCC(O)(C(=O)OCC)C(F)(F)F ; C=CCC(OCc1ccccc1)(C(=O)OCC)C(F)(F)F]

To a solution of ethyl 2-hydroxy-2-(trifluoromethyl)pent-4-enoate (18.56 g, 83.105 mmol) in DMF (100 mL) was added NaH (5.3 g, 60% w/w, 132.51 mmol) at 0° C. The reaction was stirred for 15 minutes and benzyl bromide (21.14 g, 15 mL, 121.12 mol) and tetrabutyl ammonium iodide (8.5 g, 23.012 mmol) were added. The mixture was stirred at room temperature overnight. The reaction was quenched with water (300 mL) and extracted with ethyl acetate (3×300 mL) before being washed with brine (500 mL) and dried over sodium sulfate. Purification by silica gel chromatography (20% to 60% dichloromethane in hexanes) provided ethyl 2-benzyloxy-2-(trifluoromethyl)pent-4-enoate (22.01 g, 70%) as a colorless oil. ¹H NMR (250 MHz, CDCl₃) δ 7.55-7.25 (m, 5H), 6.00-5.80 (m, 1H), 5.30-5.10 (m, 2H), 4.86 (d, J=10.5 Hz, 1H), 4.68 (d, J=10.5 Hz, 1H), 4.33 (q, J=7.0 Hz, 2H), 2.81 (d, J=7.0 Hz, 2H), 1.34 (t, J=7.1 Hz, 3H) ppm. ESI-MS m/z calc. 302.113, found 303.5 (M+1)⁺; Retention time: 4.14 minutes (LC Method G).

### Step 3: 2-Benzyloxy-2-(trifluoromethyl)pent-4-enoic Acid

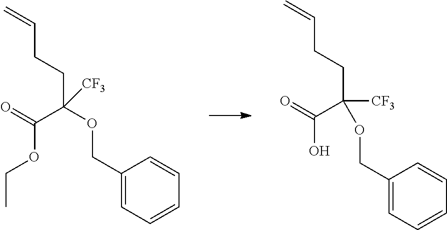 [SMILES: C=CCCC(OCc1ccccc1)(C(=O)OCC)C(F)(F)F ; C=CCCC(OCc1ccccc1)(C(=O)O)C(F)(F)F]

Into a solution of ethyl 2-benzyloxy-2-(trifluoromethyl)pent-4-enoate (28.99 g, 95.902 mmol) in methanol (150 mL) was added a solution of NaOH (7.6714 g, 191.80 mmol) in water (50 mL). The reaction mixture was stirred at 40° C. for 3 hours. The reaction mixture was concentrated under vacuum, the residue was diluted with water (200 mL) and washed with diethyl ether (200 mL). The aqueous layer was acidified with concentrated HCl to pH 1 and extracted with diethyl ether (3×200 mL). The combined organic layers were washed with brine, dried over anhydrous sodium sulfate, and concentrated under vacuum to furnish 2-benzyloxy-2-(trifluoromethyl)pent-4-enoic acid (28.04 g, 99%) as a light yellow liquid. ¹H NMR (250 MHz, CDCl₃) δ 7.55-7.28 (m, 5H), 5.97-5.69 (m, 1H), 5.33-5.17 (m, 2H), 4.95-4.66 (m, 2H), 2.91 (d, J=7.1 Hz, 2H) ppm.

### Step 4: tert-Butyl N-[[2-benzyloxy-2-(trifluoromethyl)pent-4-enoyl]amino]carbamate

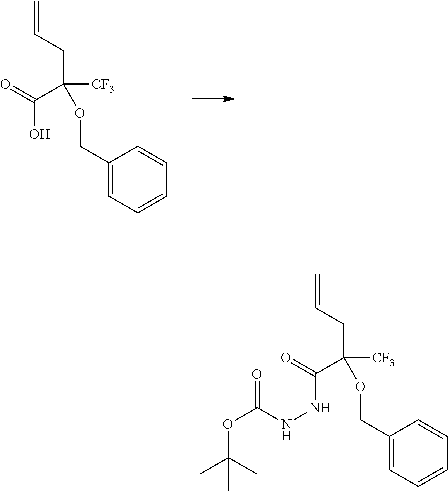 [SMILES: C=CCC(OCc1ccccc1)(C(=O)O)C(F)(F)F ; C=CCC(OCc1ccccc1)(C(=O)NNC(=O)OC(C)(C)C)C(F)(F)F]

To a solution of 2-benzyloxy-2-(trifluoromethyl)pent-4-enoic acid (300 g, 1.094 mol) in DMF (2 L) was added HATU (530 g, 1.394 mol) and DIEA (400 mL, 2.296 mol) and the mixture was stirred at ambient temperature for 10 min. To the mixture was added tert-butyl N-aminocarbamate (152 g, 1.150 mol) and the mixture stirred at ambient temperature for 36 h. The reaction was quenched with cold water (4 L) and the mixture extracted 2× with ethyl acetate (2 L). The organic phase was washed brine, dried over MgSO₄, filtered and concentrated in vacuo. Purification by silica gel chromatography (0% to 40% ethyl acetate/hexanes) provided tert-butyl N-[[2-benzyloxy-2-(trifluoromethyl)pent-4-enoyl]amino]carbamate (386.49 g, 91%) as an oil which slowly crystallized to an off-white solid. ¹H NMR (400 MHz, DMSO) δ 10.00 (d, J=37.9 Hz, 1H), 8.93 (s, 1H), 7.46-7.39 (m, 2H), 7.38-7.29 (m, 3H), 6.01-5.64 (m, 1H), 5.32 (d, J=17.1 Hz, 1H), 5.17 (d, J=10.1 Hz, 1H), 4.77 (s, 2H), 2.96 (m, 2H), 1.39 (d, J=17.3 Hz, 9H) ppm. ESI-MS m/z calc. 388.16098, found 389.0 (M+1)⁺; Retention time: 2.51 minutes (LC Method D).

### Step 5: 2-Benzyloxy-2-(trifluoromethyl)pent-4-enehydrazide (Hydrochloride Salt)

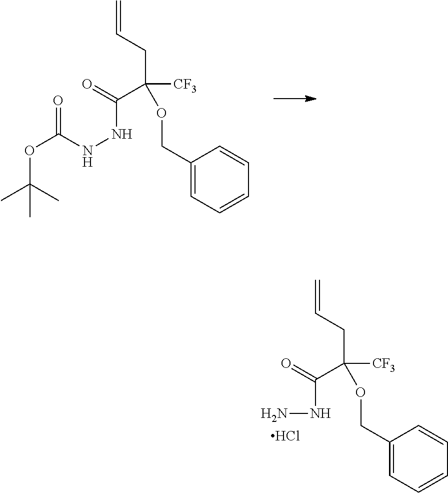 [SMILES: C=CCC(OCc1ccccc1)(C(=O)NNC(=O)OC(C)(C)C)C(F)(F)F ; Cl ; C=CCC(OCc1ccccc1)(C(=O)NN)C(F)(F)F]

To a solution of tert-butyl N-[[2-benzyloxy-2-(trifluoromethyl)pent-4-enoyl]amino]carbamate (98.5 g, 240.94 mmol) in dichloromethane (400 mL) was added HCl in dioxane (200 mL of 4 M, 800 mmol). The mixture was stirred at room temperature for 2 hours, concentrated and co-evaporated with dichloromethane and hexanes to give 2-benzyloxy-2-(trifluoromethyl)pent-4-enehydrazide (hydrochloride salt) (81.15 g, 97%) as an off white solid. ¹H NMR (500 MHz, DMSO-d₆) δ 11.07 (s, 1H), 7.70-7.16 (m, 5H), 5.87-5.61 (m, 1H), 5.45-5.09 (m, 2H), 4.79 (s, 2H), 3.6-3.4 (m, 2H), 3.23-3.07 (m, 1H), 3.04-2.87 (m, 1H) ppm. ESI-MS m/z calc. 288.10855, found 289.2 (M+1)⁺; Retention time: 2.0 minutes (LC Method H).

### Intermediate 14: Preparation of tert-butyl N-[2-[5-[1-benzyloxy-1-(trifluoromethyl)but-3-enyl]-1,3,4-oxadiazol-2-yl]-6-bromo-5-(trifluoromethyl)-3-pyridyl]carbamate

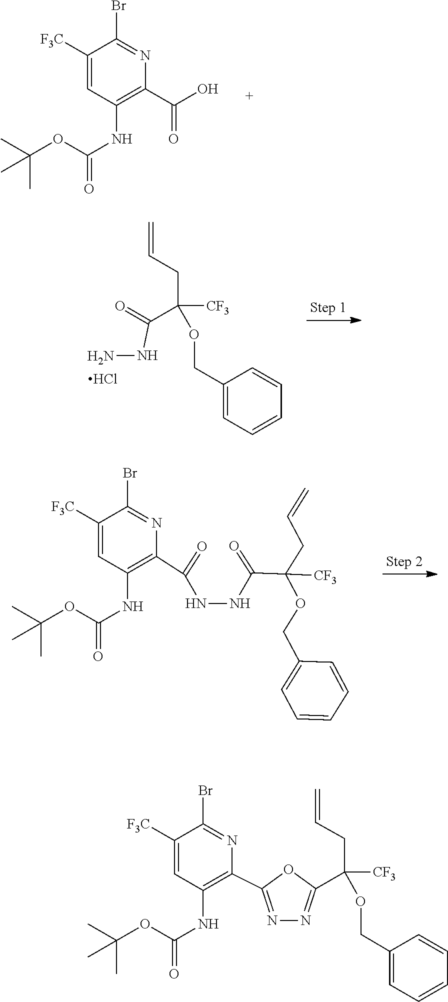 [SMILES: CC(C)(C)OC(=O)Nc1cc(C(F)(F)F)c(Br)nc1C(=O)O ; Cl ; C=CCC(OCc1ccccc1)(C(=O)NN)C(F)(F)F ; C=CCC(OCc1ccccc1)(C(=O)NNC(=O)c1nc(Br)c(C(F)(F)F)cc1NC(=O)OC(C)(C)C)C(F)(F)F ; C=CCC(OCc1ccccc1)(c1nnc(-c2nc(Br)c(C(F)(F)F)cc2NC(=O)OC(C)(C)C)o1)C(F)(F)F]

### Step 1: tert-Butyl N-[2-[[[2-benzyloxy-2-(trifluoromethyl)pent-4-enoyl]amino]carbamoyl]-6-bromo-5-(trifluoromethyl)-3-pyridyl]carbamate

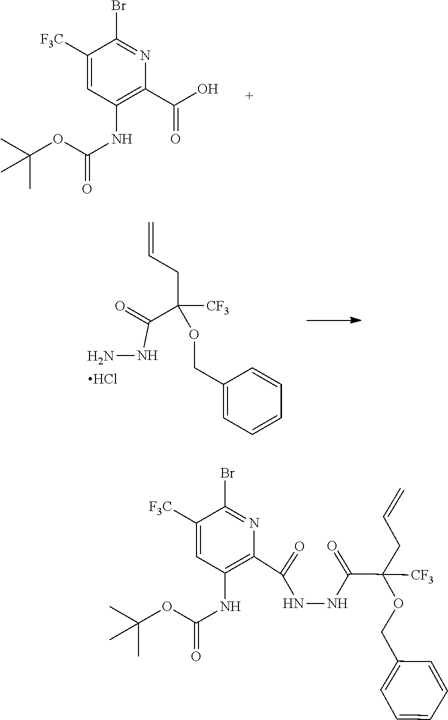 [SMILES: CC(C)(C)OC(=O)Nc1cc(C(F)(F)F)c(Br)nc1C(=O)O ; Cl ; C=CCC(OCc1ccccc1)(C(=O)NN)C(F)(F)F ; C=CCC(OCc1ccccc1)(C(=O)NNC(=O)c1nc(Br)c(C(F)(F)F)cc1NC(=O)OC(C)(C)C)C(F)(F)F]

To a mixture of 6-bromo-3-(tert-butoxycarbonylamino)-5-(trifluoromethyl)pyridine-2-carboxylic acid (53 g, 137.6 mmol) and 2-benzyloxy-2-(trifluoromethyl)pent-4-enehydrazide (hydrochloride salt) (55 g, 169.4 mmol) in ethyl acetate (500 mL) at ambient temperature was added pyridine (44 mL, 544 mmol). To the mixture was added T3P (111 g of 50% w/w, 174.4 mmol) and the reaction mixture stirred at ambient temperature for 12 h. The reaction was quenched with slow addition of NaOH (35 g of 50% w/w, 437.5 mmol) in water (500 mL) and the mixture stirred for 15 min. The organic phase was separated and the aqueous phase extracted with ethyl acetate (500 mL). The combined organic phases washed with HCl (250 mL of 1 M, 250 mmol), brine, dried over MgSO₄, filtered and concentrated in vacuo. Purification by silica gel chromatography (0% to 20% ethyl acetate in hexanes) provided tert-butyl N-[2-[[[2-benzyloxy-2-(trifluoromethyl)pent-4-enoyl]amino]carbamoyl]-6-bromo-5-(trifluoromethyl)-3-pyridyl]carbamate (66 g, 73%) as a pale pink solid. ¹H NMR (400 MHz, DMSO) δ 11.11 (s, 1H), 10.91 (s, 1H), 10.40 (s, 1H), 9.16 (s, 1H), 7.47 (d, J=6.9 Hz, 2H), 7.42-7.29 (m, 3H), 5.91 (m, J=17.1, 10.6, 7.1 Hz, 1H), 5.37 (dd, J=17.2, 1.9 Hz, 1H), 5.22 (dd, J=10.4, 1.8 Hz, 1H), 4.85 (d, J=2.1 Hz, 2H), 3.20-2.91 (m, 2H), 1.50 (s, 9H) ppm. ESI-MS m/z calc. 654.09125, found 657.0 (M+1)⁺; Retention time: 3.49 minutes (LC Method FF).

### Step 2: tert-Butyl N-[2-[5-[1-benzyloxy-1-(trifluoromethyl)but-3-enyl]-1,3,4-oxadiazol-2-yl]-6-bromo-5-(trifluoromethyl)-3-pyridyl]carbamate

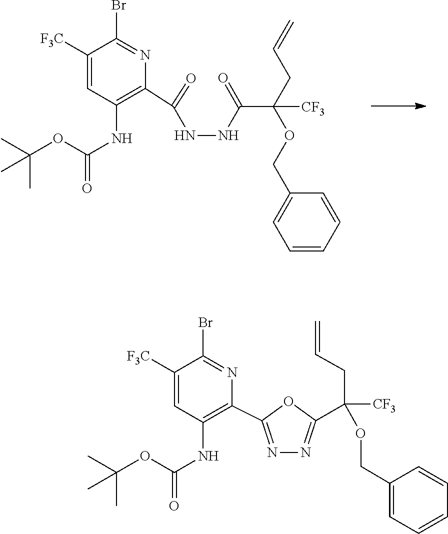 [SMILES: C=CCC(OCc1ccccc1)(C(=O)NNC(=O)c1nc(Br)c(C(F)(F)F)cc1NC(=O)OC(C)(C)C)C(F)(F)F ; C=CCC(OCc1ccccc1)(c1nnc(-c2nc(Br)c(C(F)(F)F)cc2NC(=O)OC(C)(C)C)o1)C(F)(F)F]

A solution of tert-butyl N-[2-[[[2-benzyloxy-2-(trifluoromethyl)pent-4-enoyl]amino]carbamoyl]-6-bromo-5-(trifluoromethyl)-3-pyridyl]carbamate (2.15 g, 3.2641 mmol) and DIEA (1.12 g, 1.5 mL, 8.6117 mmol) in acetonitrile (43 mL) was heated at 50° C., then p-toluenesulfonyl chloride (765 mg, 4.0127 mmol) was added portion wise at 50° C. Resultant mixture was stirred at 70° C. for 2 hours. Reaction mixture was cooled, basified with a saturated solution of sodium bicarbonate (100 mL) and extracted with ethyl acetate (3×50 mL). Combined organic layers were washed with brine (50 mL), dried over sodium sulfate, filtered and concentrated. purification by silica gel chromatography (0% to 10% of ethyl acetate in heptanes) afforded tert-butyl N-[2-[5-[1-benzyloxy-1-(trifluoromethyl)but-3-enyl]-1,3,4-oxadiazol-2-yl]-6-bromo-5-(trifluoromethyl)-3-pyridyl]carbamate (1.7 g, 80%) as a yellow viscous oil. ¹H NMR (300 MHz, CDCl₃) δ 10.18 (br. s, 1H), 9.33 (br. s, 1H), 7.53-7.27 (m, 5H), 6.00-5.83 (m, 1H), 5.32-5.13 (m, 2H), 4.86-4.76 (m, 1H), 4.73-4.64 (m, 1H), 3.27-3.11 (m, 2H), 1.55 (s, 9H) ppm. ¹⁹F NMR (282 MHz, CDCl₃) δ−63.78 (s, 3F), −72.93 (s, 3F) ppm. Retention time: 2.7 minutes (LC Method O).

### Intermediate 15: Preparation of tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate

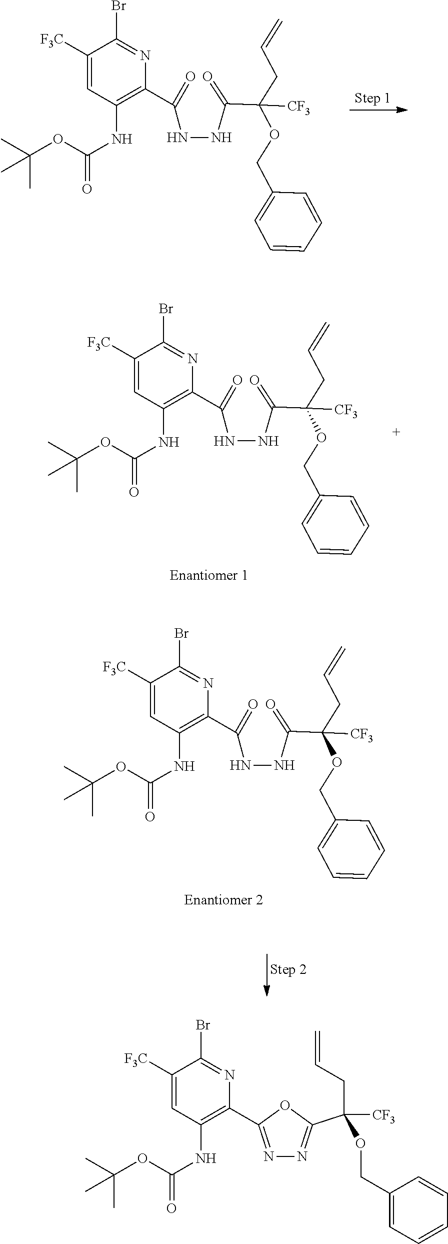 [SMILES: C=CCC(OCc1ccccc1)(C(=O)NNC(=O)c1nc(Br)c(C(F)(F)F)cc1NC(=O)OC(C)(C)C)C(F)(F)F ; C=CC[C@](OCc1ccccc1)(C(=O)NNC(=O)c1nc(Br)c(C(F)(F)F)cc1NC(=O)OC(C)(C)C)C(F)(F)F ; C=CC[C@@](OCc1ccccc1)(C(=O)NNC(=O)c1nc(Br)c(C(F)(F)F)cc1NC(=O)OC(C)(C)C)C(F)(F)F ; C=CC[C@@](OCc1ccccc1)(c1nnc(-c2nc(Br)c(C(F)(F)F)cc2NC(=O)OC(C)(C)C)o1)C(F)(F)F]

### Step 1: tert-Butyl N-[2-[[[(2S)-2-benzyloxy-2-(trifluoromethyl)pent-4-enoyl]amino]carbamoyl]-6-bromo-5-(trifluoromethyl)-3-pyridyl]carbamate and tert-butyl N-[2-[[[(2R)-2-benzyloxy-2-(trifluoromethyl)pent-4-enoyl]amino]carbamoyl]-6-bromo-5-(trifluoromethyl)-3-pyridyl]carbamate

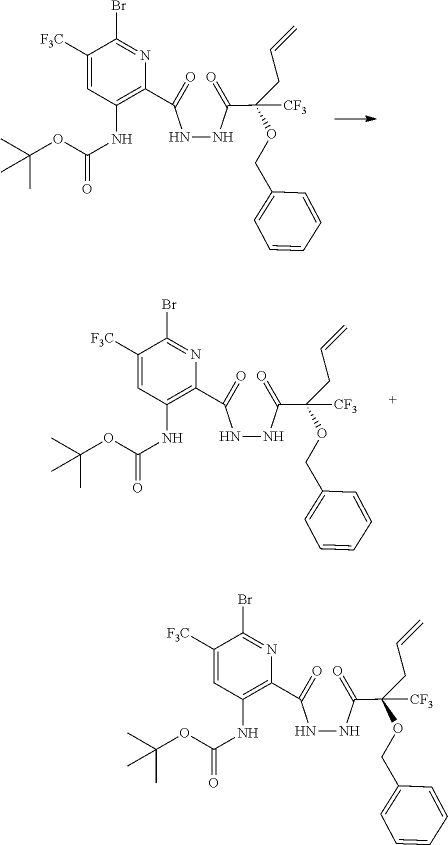 [SMILES: C=CC[C@](OCc1ccccc1)(C(=O)NNC(=O)c1nc(Br)c(C(F)(F)F)cc1NC(=O)OC(C)(C)C)C(F)(F)F ; C=CC[C@](OCc1ccccc1)(C(=O)NNC(=O)c1nc(Br)c(C(F)(F)F)cc1NC(=O)OC(C)(C)C)C(F)(F)F ; C=CC[C@@](OCc1ccccc1)(C(=O)NNC(=O)c1nc(Br)c(C(F)(F)F)cc1NC(=O)OC(C)(C)C)C(F)(F)F]

The racemic tert-butyl N-[2-[[[2-benzyloxy-2-(trifluoromethyl)pent-4-enoyl]amino]carbamoyl]-6-bromo-5-(trifluoromethyl)-3-pyridyl]carbamate (24.5 g, 37.38 mmol) was purified by preparative chiral SFC by 500 μL injections of a 32 mg/mL solution onto a ChiralPak IC (250×21.2 mm), 5 m column eluted at 40° C. at 70 mL/min with 8% MeOH (20 mM NH₃) and 92% CO₂. First eluting enantiomer-1 (peak 1 at room temperature 4.17 min) to provided tert-butyl N-[2-[[[(2S)-2-benzyloxy-2-(trifluoromethyl)pent-4-enoyl]amino]carbamoyl]-6-bromo-5-(trifluoromethyl)-3-pyridyl]carbamate (11.73 g, 96%) ¹H NMR (400 MHz, Chloroform-d) δ 10.59 (s, 1H), 9.83 (s, 1H), 9.28 (s, 1H), 9.02 (d, J=29.6 Hz, 1H), 7.48-7.33 (m, 5H), 5.96-5.77 (m, 1H), 5.41 (d, J=1.6 Hz, 1H), 5.36-5.29 (m, 1H), 4.86 (s, 2H), 3.19 (dd, J=15.5, 5.9 Hz, 1H), 3.03 (dd, J=15.5, 7.8 Hz, 1H), 1.53 (s, 9H) ppm; ¹⁹F NMR (376 MHz, Chloroform-d) δ−63.89, −73.76 ppm. ESI-MS m/z calc. 654.09125, found 655.3 (M+1)⁺; Retention time: 0.53 minutes (LC Method T).

The later eluting enantiomer 2 (peak 2 at room temperature 6.63 min) provided tert-butyl N-[2-[[[(2R)-2-benzyloxy-2-(trifluoromethyl)pent-4-enoyl]amino]carbamoyl]-6-bromo-5-(trifluoromethyl)-3-pyridyl]carbamate (11.62 g, 95%). ¹H NMR (400 MHz, Chloroform-d) δ 10.59 (s, 1H), 9.74 (s, 1H), 9.28 (s, 1H), 9.06 (s, 1H), 7.39 (d, J=4.4 Hz, 5H), 6.02-5.79 (m, 1H), 5.44-5.36 (m, 1H), 5.34 (dd, J=10.3, 1.3 Hz, 1H), 4.91-4.81 (m, 2H), 3.19 (dd, J=15.4, 5.8 Hz, 1H), 3.03 (dd, J=15.5, 7.8 Hz, 1H), 1.53 (s, 9H) ppm; ¹⁹F NMR (376 MHz, Chloroform-d) δ−63.89, −73.76 ppm. ESI-MS m/z calc. 654.09125, found 657.2 (M+1)⁺; Retention time: 0.53 minutes (LC Method T).

### Step 2: tert-Butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate

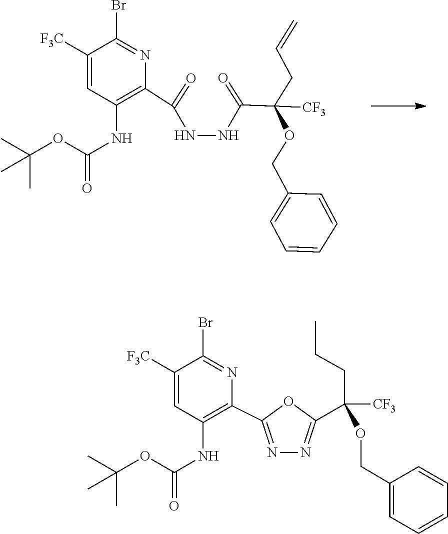 [SMILES: C=CC[C@@](OCc1ccccc1)(C(=O)NNC(=O)c1nc(Br)c(C(F)(F)F)cc1NC(=O)OC(C)(C)C)C(F)(F)F ; CCC[C@@](OCc1ccccc1)(c1nnc(-c2nc(Br)c(C(F)(F)F)cc2NC(=O)OC(C)(C)C)o1)C(F)(F)F]

tert-Butyl N-[2-[[[(2R)-2-benzyloxy-2-(trifluoromethyl)pent-4-enoyl]amino]carbamoyl]-6-bromo-5-(trifluoromethyl)-3-pyridyl]carbamate (enantiomer 2; 24.97 g, 38.10 mmol) was dissolved in anhydrous acetonitrile (200 mL) under nitrogen, making a clear yellow solution. DIEA (19.91 mL, 114.3 mmol) was added, and the solution turned orange. The solution was heated to 70° C., then p-TsCl (7.99 g, 41.91 mmol) was added in 3 portions at 30 min intervals and heated for about 3 h. The reaction mixture was cooled to room temperature and evaporated majority of the MeCN via rotary evaporation at 45° C. Added 145 mL MTBE, followed by a solution of citric acid (11.0 g, 57.25 mmol) in 250 mL water, stirred, then added 73 mL hexanes. Separated the layers and water layer extracted with MTBE. Combined the organic layers dried over MgSO₄, concentrated on rotovap at 45° C. Purification by silica gel chromatography (15% to 80% of 10% ethyl acetate/Hexanes solution in hexanes) provided tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (20.47 g, 84%). ¹H NMR (400 MHz, Chloroform-d) δ 10.18 (s, 1H), 9.34 (s, 1H), 7.48 (d, J=7.1 Hz, 2H), 7.39 (t, J=7.5 Hz, 2H), 7.31 (t, J=7.3 Hz, 1H), 6.00-5.81 (m, 1H), 5.25 (d, J=17.1, 1.6 Hz, 1H), 5.20 (d, J=10.1, 1.5 Hz, 1H), 4.82 (d, J=10.6 Hz, 1H), 4.70 (d, J=10.6 Hz, 1H), 3.30-3.09 (m, 2H), 1.56 (s, 9H) ppm. ESI-MS m/z calc. 636.0807, found 637.3 (M+1)⁺; Retention time: 3.81 minutes (LC Method FF).

### Intermediate 16: Preparation of 6-[5-[1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic Acid

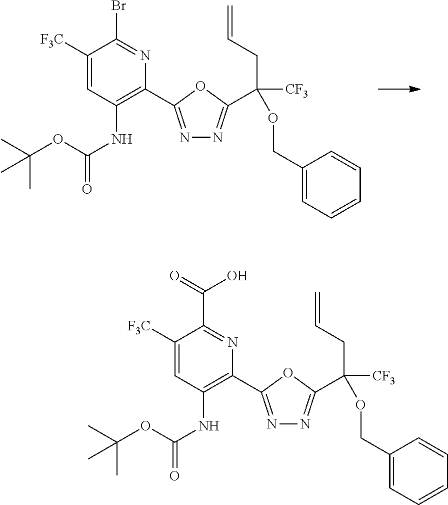 [SMILES: C=CCC(OCc1ccccc1)(c1nnc(-c2nc(Br)c(C(F)(F)F)cc2NC(=O)OC(C)(C)C)o1)C(F)(F)F ; C=CCC(OCc1ccccc1)(c1nnc(-c2nc(C(=O)O)c(C(F)(F)F)cc2NC(=O)OC(C)(C)C)o1)C(F)(F)F]

To a solution of tert-butyl N-[2-[5-[1-benzyloxy-1-(trifluoromethyl)but-3-enyl]-1,3,4-oxadiazol-2-yl]-6-bromo-5-(trifluoromethyl)-3-pyridyl]carbamate (570 mg, 0.8943 mmol) in ether (12 mL) at −78° C. was added n-BuLi (830 μL of 2.5 M, 2.075 mmol) and the mixture stirred at −78° C. for 15 min then warmed to 0° C. in 5 seconds and small pieces of solid CO₂ were added. The mixture was stirred at 0° C. for 15 min. The mixture was diluted with ethyl acetate and 2.8 mL of 1 M citric acid was added changing the pH to 4 by paper. The organic layer was separated and the aqueous layer extracted with ethyl acetate. The combined organic layers were washed with water, brine, dried (MgSO₄) and evaporated. Purification by silica gel chromatography (10-20% ethyl acetate in hexanes) provided 6-[5-[1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid (326 mg, 54%). ¹H NMR (400 MHz, Chloroform-d) δ 10.38 (s, 1H), 9.55 (s, 1H), 7.33 (s, 5H), 5.90 (s, 1H), 5.31-5.11 (m, 2H), 4.88-4.76 (m, 1H), 4.68 (s, 1H), 3.21 (d, J=7.6 Hz, 2H), 1.58 (s, 9H) ppm; ¹⁹F NMR (376 MHz, Chloroform-d) δ−61.04, −73.19 ppm. ESI-MS m/z calc. 602.16003, found 603.1 (M+1)⁺; Retention time: 0.8 minutes (LC Method S).

### Intermediate 17: Preparation of 6-[5-[(1R)-1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic Acid

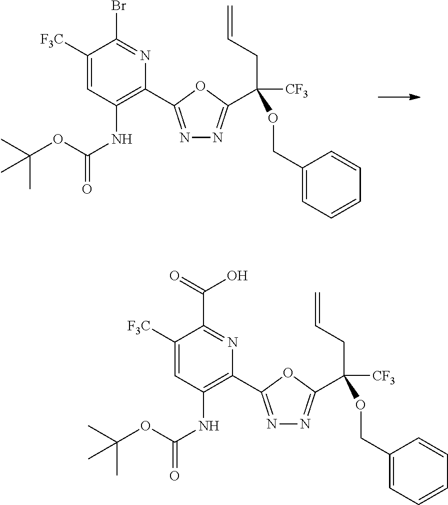 [SMILES: C=CC[C@@](OCc1ccccc1)(c1nnc(-c2nc(Br)c(C(F)(F)F)cc2NC(=O)OC(C)(C)C)o1)C(F)(F)F ; C=CC[C@@](OCc1ccccc1)(c1nnc(-c2nc(C(=O)O)c(C(F)(F)F)cc2NC(=O)OC(C)(C)C)o1)C(F)(F)F]

To tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (4.7 g, 7.005 mmol) in ether (89 mL) at −78° C. was added n-BuLi (6.2 mL of 2.5 M, 15.50 mmol) and the mixture stirred at −78° C. for 20 min then warmed to 0° C. in 5 seconds and small pieces of solid CO₂ were added. The mixture was stirred at 0° C. for 15 min. The mixture was diluted with citric acid (22 mL of 1 M, 22 mmol) as a solution in water changing the pH to 4 by paper and ethyl acetate was added. The organic layer was separated and washed with water, brine, dried (MgSO₄) and evaporated. Purification by silica gel chromatography (5% to 50% of a solution 10% MeOH in ethyl acetate and hexanes) provided 6-[5-[(1R)-1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid (2.1 g, 50%). ¹H NMR (400 MHz, Chloroform-d) δ 10.43 (s, 1H), 9.61 (s, 1H), 7.42-7.31 (m, 4H), 7.30-7.26 (m, 1H), 5.94 (m, J=17.1, 7.2 Hz, 1H), 5.33-5.20 (m, 2H), 4.84 (d, J=11.0 Hz, 1H), 4.71 (d, J=11.0 Hz, 1H), 3.24 (m, J=15.2, 7.0 Hz, 2H), 1.59 (s, 9H) ppm; ¹⁹F NMR (376 MHz, Chloroform-d) δ−61.10, −73.16 ppm. ESI-MS m/z calc. 602.16003, found 603.2 (M+1)⁺; Retention time: 0.4 minutes (LC Method T).

### Intermediate 18: Preparation of 2-benzyloxy-6-(1,3-dioxolan-2-yl)-2-(trifluoromethyl)hexanohydrazide

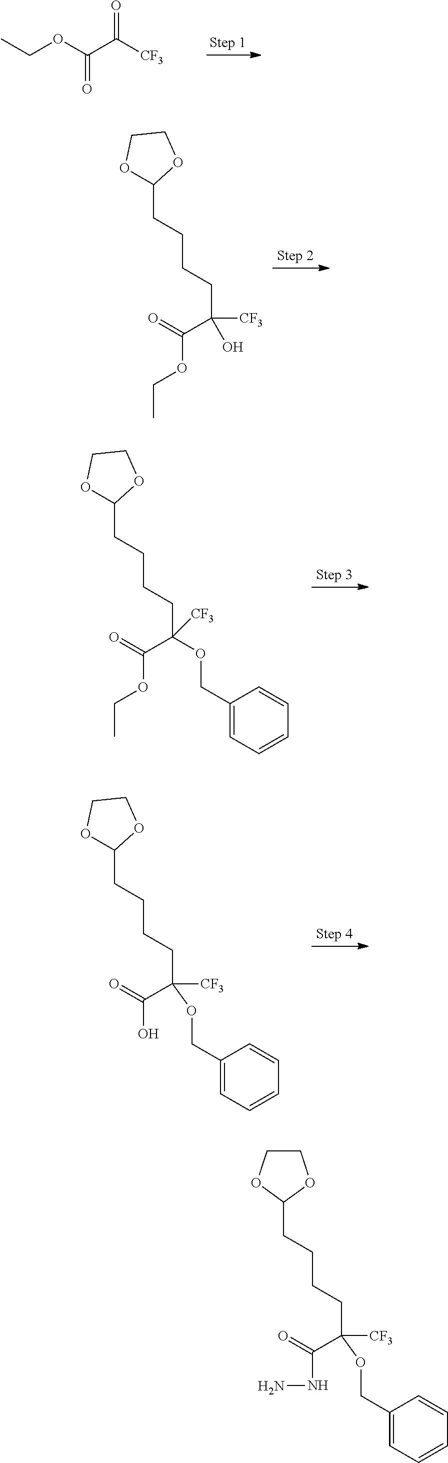

### Step 1: Ethyl 6-(1,3-dioxolan-2-yl)-2-hydroxy-2-(trifluoromethyl)hexanoate

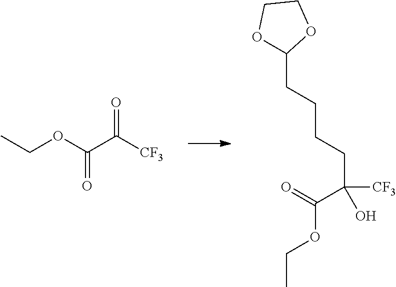 [SMILES: CCOC(=O)C(=O)C(F)(F)F ; CCOC(=O)C(O)(CCCCC1OCCO1)C(F)(F)F]

To a dried 250-mL three-neck flask equipped with a reflux condenser under nitrogen was charged with magnesium (2.2 g, 83.584 mmol), iodine (5 mg, 0.0197 mmol) was added. The mixture was heated neat at 55° C. with stirring. A solution of 2-(4-bromobutyl)-1,3-dioxolane (14.35 g, 68.634 mmol) in THF (115 mL) was added dropwise over 20 min, so that the reaction mixture was continuously boiling. After addition, the dark reaction mixture was heated at 78° C. for 1 h and cooled to room temperature. The resultant bromo-[4-(1,3-dioxolan-2-yl)butyl]magnesium (in THF) (115 mL of 0.6 M, 69 mmol) was added to a solution of ethyl 3,3,3-trifluoro-2-oxo-propanoate (11.547 g, 9 mL, 67.889 mmol) in diethyl ether (120 mL) at −78° C. slowly dropwise over a period of 20 minutes. The mixture was stirred at −78° C. for 20 min. The dry ice-acetone bath was removed. The reaction mixture was slowly warm up to 10° C. in 20 minutes, added to a mixture of 1N aqueous hydrochloric acid (100 mL) and crushed ice (60 g). The two layers were separated. The organic layer was concentrated, and the residue was combined with aqueous phase and extracted with ethyl acetate (3×100 mL). The organic layers were washed with aqueous saturated sodium bicarbonate solution (100 mL) and brine (100 mL) and dried over sodium sulfate. The mixture was filtered and concentrated to give as a yellow oil, ethyl 6-(1,3-dioxolan-2-yl)-2-hydroxy-2-(trifluoromethyl)hexanoate (21.52 g, 93%). ¹H NMR (400 MHz, CDCl₃) δ 4.87-4.81 (m, 1H), 4.57 (br s, 1H), 4.43-4.29 (m, 2H), 3.99-3.93 (m, 2H), 3.88-3.82 (m, 2H), 2.03-1.95 (m, 1H), 1.92-1.82 (m, 1H), 1.71-1.62 (m, 2H), 1.61-1.52 (m, 1H), 1.51-1.37 (m, 3H), 1.34 (t, J=7.1 Hz, 3H) ppm. ¹⁹F NMR (377 MHz, CDCl₃) δ−78.66 (s, 3F) ppm. ESI-MS m/z calc. 300.11847, found 323.2 (M+23)⁺; Retention time: 1.71 minutes (LC Method Z).

### Step 2: Ethyl 2-benzyloxy-6-(1,3-dioxolan-2-yl)-2-(trifluoromethyl)hexanoate

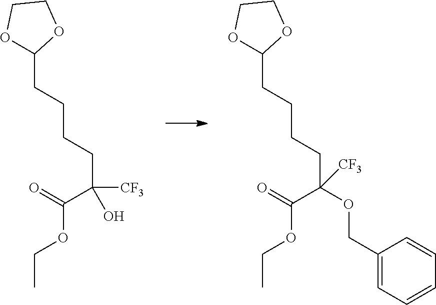 [SMILES: CCOC(=O)C(O)(CCCCC1OCCO1)C(F)(F)F ; CCOC(=O)C(CCCCC1OCCO1)(OCc1ccccc1)C(F)(F)F]

To a solution of ethyl 6-(1,3-dioxolan-2-yl)-2-hydroxy-2-(trifluoromethyl)hexanoate (21.5 g, 63.010 mmol) in DMF (90 mL) at 0° C. was added NaH (4.6 g, 115.01 mmol) in portion. The mixture was stirred at 0° C. for 30 min. Bromoethylbenzene (21.139 g, 14.7 mL, 123.60 mmol) was added dropwise, followed by tetrabutylammonium iodide (4.2 g, 11.371 mmol). The mixture was slowly warmed to room temperature and stirred at room temperature overnight. Ammonium chloride (2.9 g, 54.215 mmol) was added. The mixture was stirred at room temperature for 10 min. Water (500 mL) was added, followed by a mixture of MTBE and heptanes 2:1 (200 mL). The two layers was separated, and the aqueous phase was extracted with a mixture of MTBE and heptane (2:1, 2×200 mL). The combined organic layers were washed with water (200 mL) and brine (200 mL) than dried over sodium sulfate. The mixture was filtered and concentrated under reduced pressure. Purification by silica gel chromatography using a gradient from 0% to 30% of ethyl acetate in heptanes provided as colorless oil, ethyl 2-benzyloxy-6-(1,3-dioxolan-2-yl)-2-(trifluoromethyl)hexanoate (18.4 g, 58%). ¹H NMR (400 MHz, CDCl₃) δ 7.46-7.29 (m, 5H), 4.87-4.77 (m, 2H), 4.69-4.60 (m, 1H), 4.33 (m, 2H), 3.98-3.91 (m, 2H), 3.87-3.79 (m, 2H), 2.05-1.96 (m, 2H), 1.71-1.54 (m, 3H), 1.50-1.37 (m, 3H), 1.34 (t, J=7.2 Hz, 3H) ppm. ¹⁹F NMR (377 MHz, CDCl₃) δ−70.53 (s, 3F) ppm. ESI-MS m/z calc. 390.1654, found 413.2 (M+23)⁺; Retention time: 2.02 minutes (LC Method Z).

### Step 3: 2-Benzyloxy-6-(1,3-dioxolan-2-yl)-2-(trifluoromethyl)hexanoic Acid

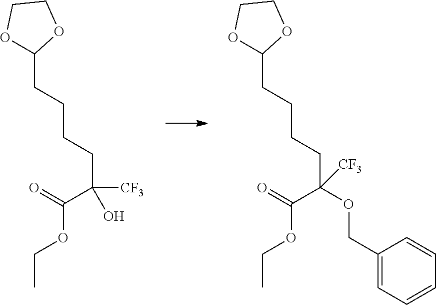 [SMILES: CCOC(=O)C(O)(CCCCC1OCCO1)C(F)(F)F ; CCOC(=O)C(CCCCC1OCCO1)(OCc1ccccc1)C(F)(F)F]

A solution of sodium hydroxide (7.5 g, 187.51 mmol) in water (75 mL) was added to a solution of ethyl 2-benzyloxy-6-(1,3-dioxolan-2-yl)-2-(trifluoromethyl)hexanoate (28.9 g, 74.028 mmol) in methanol (230 mL). The reaction was heated at 50° C. for 6 h. The reaction was concentrated to remove methanol. The crude material was diluted in water (300 mL) and the carboxylate sodium salt was washed with heptane (100 mL) and MTBE (100 mL). The aqueous solution was acidified to pH=2 with an aqueous solution of 3N HCl. The carboxylic acid was extracted with dichloromethane (4×200 mL) and dried over sodium sulfate. The solution was filtered and concentrated to give as clear oil, 2-benzyloxy-6-(1,3-dioxolan-2-yl)-2-(trifluoromethyl)hexanoic acid (27.75 g, 96%) which became solid on standing. ¹H NMR (400 MHz, CDCl₃) δ 8.64 (br s, 1H), 7.45-7.30 (m, 5H), 4.91-4.80 (m, 2H), 4.77-4.64 (m, 1H), 4.02-3.92 (m, 2H), 3.90-3.79 (m, 2H), 2.22-2.01 (m, 2H), 1.74-1.65 (m, 2H), 1.64-1.55 (m, 1H), 1.54-1.34 (m, 3H) ppm. ¹⁹F NMR (377 MHz, CDCl₃) δ−71.58 (s, 3F) ppm. ESI-MS m/z calc. 362.1341, found 361.1 (M−1)⁺; Retention time: 4.29 minutes (LC Method AA).

### Step 4: 2-Benzyloxy-6-(1,3-dioxolan-2-yl)-2-(trifluoromethyl)hexanohydrazide

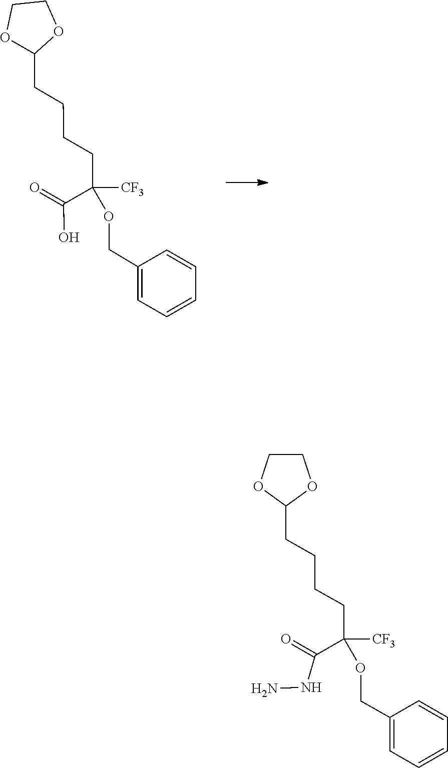 [SMILES: O=C(O)C(CCCCC1OCCO1)(OCc1ccccc1)C(F)(F)F ; NNC(=O)C(CCCCC1OCCO1)(OCc1ccccc1)C(F)(F)F]

To a solution of 2-benzyloxy-6-(1,3-dioxolan-2-yl)-2-(trifluoromethyl)hexanoic acid (16.7 g, 46.089 mmol) in DMF (175 mL) was added triethylamine (20.546 g, 28.3 mL, 203.04 mmol) and HATU (53.4 g, 140.44 mmol). The mixture was stirred for 10 min. Then, hydrazine (Water (1)) (92.880 g, 90 mL, 1.8554 mol) was added. The mixture was stirred at room temperature overnight. The mixture was diluted with water (1 L) and extracted with ethyl acetate (3×300 mL). The combined organic layer was washed with aqueous saturated sodium bicarbonate solution (300 mL), water (300 mL) and brine (300 mL). The organic layer was concentrated by evaporation under reduced pressure. Purification by silica gel chromatography using a gradient from 20% to 80% of ethyl acetate in heptanes provided as an orange oil, 2-benzyloxy-6-(1,3-dioxolan-2-yl)-2-(trifluoromethyl)hexanohydrazide (9.3 g, 48%). ¹H NMR (400 MHz, CDCl₃) δ 7.97 (br s, 1H), 7.45-7.30 (m, 5H), 4.85 (t, J=4.6 Hz, 1H), 4.75 (d, J=10.1 Hz, 1H), 4.61 (d, J=10.4 Hz, 1H), 4.02-3.92 (m, 2H), 3.91-3.77 (m, 4H), 2.40-2.26 (m, 1H), 2.19-2.06 (m, 1H), 1.75-1.64 (m, 2H), 1.61-1.40 (m, 3H), 1.38-1.30 (m, 1H) ppm. ¹⁹F NMR (377 MHz, CDCl₃) δ−73.87 (s, 3F) ppm. ESI-MS m/z calc. 376.16098, found 377.2 (M+1)⁺; Retention time: 1.68 minutes (LC Method Z).

### Intermediate 19: Preparation of methyl 6-[5-[1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate

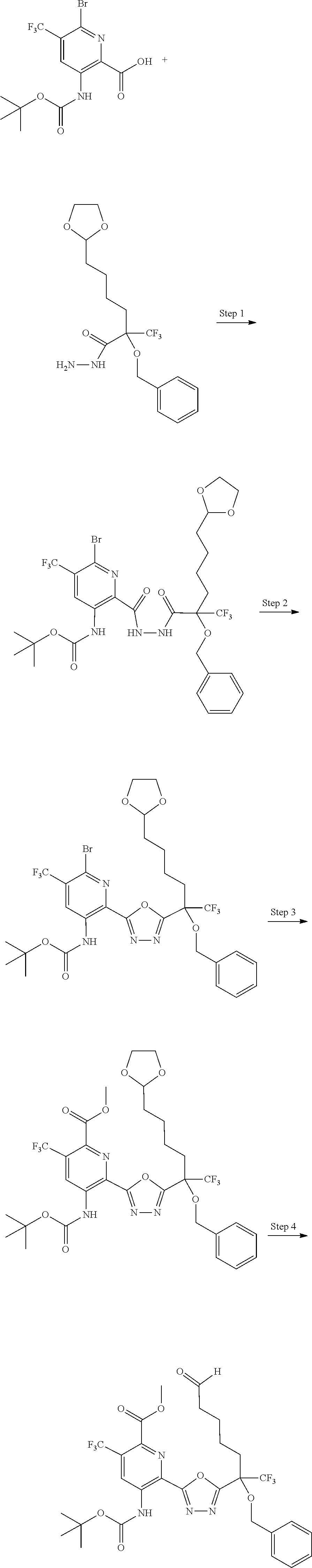 [SMILES: CC(C)(C)OC(=O)Nc1cc(C(F)(F)F)c(Br)nc1C(=O)O ; NNC(=O)C(CCCCC1OCCO1)(OCc1ccccc1)C(F)(F)F ; CC(C)(C)OC(=O)Nc1cc(C(F)(F)F)c(Br)nc1C(=O)NNC(=O)C(CCCCC1OCCO1)(OCc1ccccc1)C(F)(F)F ; CC(C)(C)OC(=O)Nc1cc(C(F)(F)F)c(Br)nc1-c1nnc(C(CCCCC2OCCO2)(OCc2ccccc2)C(F)(F)F)o1 ; COC(=O)c1nc(-c2nnc(C(CCCCC3OCCO3)(OCc3ccccc3)C(F)(F)F)o2)c(NC(=O)OC(C)(C)C)cc1C(F)(F)F ; COC(=O)c1nc(-c2nnc(C(CCCCC=O)(OCc3ccccc3)C(F)(F)F)o2)c(NC(=O)OC(C)(C)C)cc1C(F)(F)F]

### Step 1: tert-Butyl N-[2-[[[2-benzyloxy-6-(1,3-dioxolan-2-yl)-2-(trifluoromethyl)hexanoyl]amino]carbamoyl]-6-bromo-5-(trifluoromethyl)-3-pyridyl]carbamate

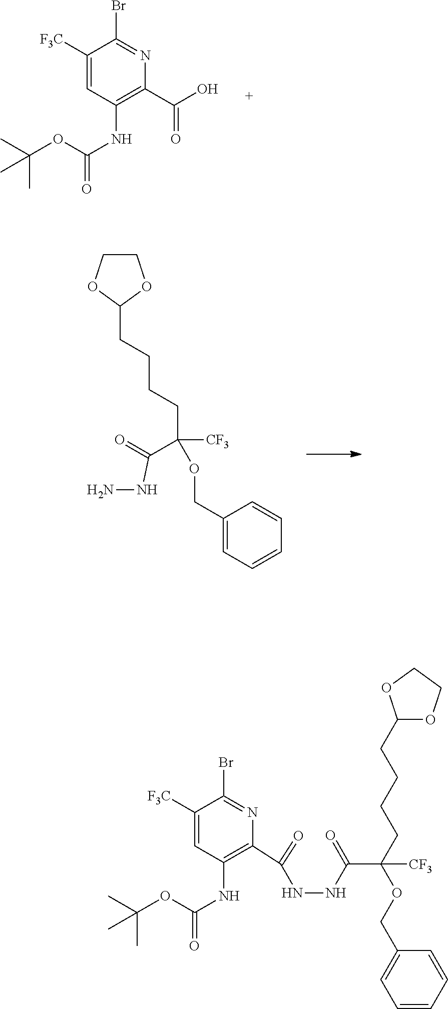 [SMILES: CC(C)(C)OC(=O)Nc1cc(C(F)(F)F)c(Br)nc1C(=O)O ; NNC(=O)C(CCCCC1OCCO1)(OCc1ccccc1)C(F)(F)F ; CC(C)(C)OC(=O)Nc1cc(C(F)(F)F)c(Br)nc1C(=O)NNC(=O)C(CCCCC1OCCO1)(OCc1ccccc1)C(F)(F)F]

To a mixture of 6-bromo-3-(tert-butoxycarbonylamino)-5-(trifluoromethyl)pyridine-2-carboxylic acid (11.2 g, 29.08 mmol) and 2-benzyloxy-6-(1,3-dioxolan-2-yl)-2-(trifluoromethyl)hexanohydrazide (12.0 g, 31.88 mmol) in ethyl acetate (140 mL) at ambient temperature was added pyridine (9.5 mL, 117.5 mmol). To this solution was added T3P (24 mL of 50% w/v, 37.71 mmol) and the reaction mixture stirred at ambient temperature for 16 hours. The reaction was quenched with the slow addition of NaOH (50% in water) (7.3 mL of 50% w/v, 91.26 mmol) in water (120 mL) and the mixture stirred for 5 min in an ice bath and then at room temperature for 1 h. The organic phase was separated, and the aqueous phase extracted with ethyl acetate (100 mL). The combined organic phases washed with HCl (50 mL of 1 M), brine, dried (MgSO₄), filtered and evaporated. Purification by silica gel chromatography using a gradient from 0% to 25% ethyl acetate in hexanes provided tert-butyl N-[2-[[[2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (16.4 g, 76%). ESI-MS m/z calc. 742.1437, found 645.2 (M+1)⁺; Retention time: 0.43 minutes (LC Method T).

### Step 2: tert-Butyl N-[2-[5-[1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate

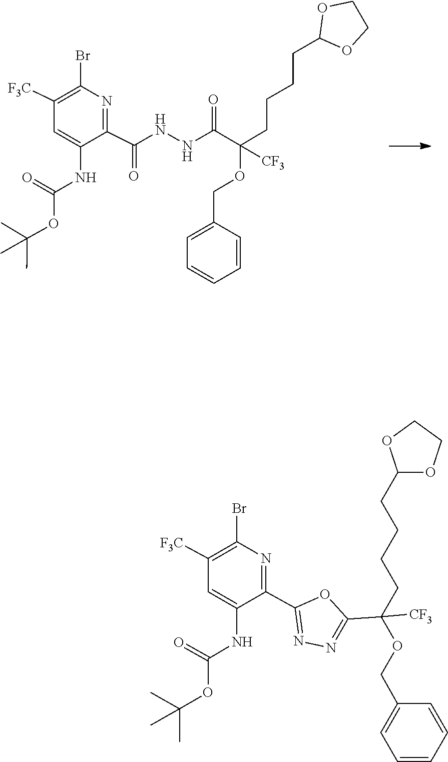 [SMILES: CC(C)(C)OC(=O)Nc1cc(C(F)(F)F)c(Br)nc1C(=O)NNC(=O)C(CCCCC1OCCO1)(OCc1ccccc1)C(F)(F)F ; CC(C)(C)OC(=O)Nc1cc(C(F)(F)F)c(Br)nc1-c1nnc(C(CCCCC2OCCO2)(OCc2ccccc2)C(F)(F)F)o1]

To a solution of tert-butyl N-[2-[[[2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (2.298 g, 3.0908 mmol) and DIEA (1.2020 g, 1.62 mL, 9.3003 mmol) in acetonitrile (37 mL) was added tosyl chloride (707 mg, 3.7084 mmol) at 50° C. The reaction was stirred at 70° C. for 2 hours. The reaction was cooled to room temperature and then it was diluted with ethyl acetate (150 mL). The organic solution was washed with saturated ammonium chloride (50 mL) and brine (50 mL), dried over anhydrous sodium sulfate, and concentrated under vacuum. The residue was purified by silica gel chromatography using a gradient from 0% to 30% ethyl acetate in hexane to furnish as a yellow foam, tert-butyl N-[2-[5-[1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate (2.026 g, 87%). ESI-MS m/z calc. 724.1331, found 725.3 (M+1)⁺; Retention time: 4.33 minutes (LC Method G).

### Step 3: Methyl 6-[5-[1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate

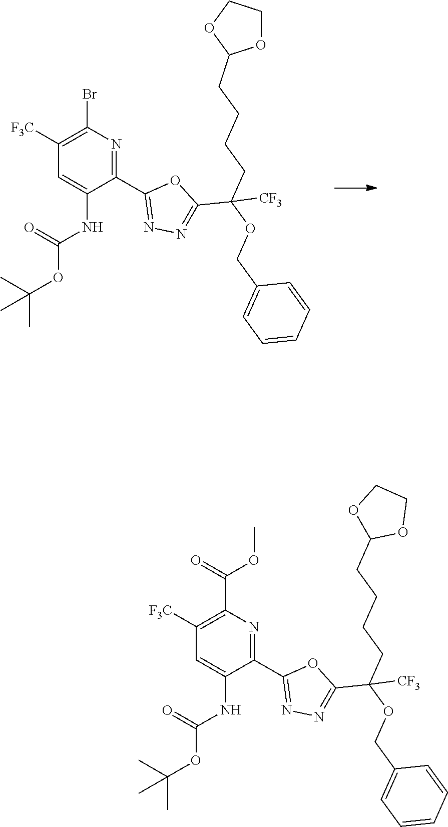 [SMILES: CC(C)(C)OC(=O)Nc1cc(C(F)(F)F)c(Br)nc1-c1nnc(C(CCCCC2OCCO2)(OCc2ccccc2)C(F)(F)F)o1 ; COC(=O)c1nc(-c2nnc(C(CCCCC3OCCO3)(OCc3ccccc3)C(F)(F)F)o2)c(NC(=O)OC(C)(C)C)cc1C(F)(F)F]

Into a solution of tert-butyl N-[2-[5-[1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate (1.925 g, 2.5473 mmol) in methanol (50 mL) was added TEA (798.60 mg, 1.1 mL, 7.8921 mmol). The reaction was purged with argon for 2 minutes. Pd(dppf)Cl₂ (186 mg, 0.2542 mmol) was added to the reaction mixture. The reaction was then stirred at 80° C. under 50 psi carbon monoxide for 6 hours. The reaction was cooled to rt, and then methanol was removed under vacuum. The residue was diluted with ethyl acetate (50 mL) and water (50 mL). Two layers were separated, and the aqueous layer was extracted with ethyl acetate (2×50 mL). The combined organic layers were washed with brine (50 mL), dried over anhydrous magnesium sulfate, and concentrated under vacuum. The residue was purified by silica gel chromatography using a gradient from 0% to 40% ethyl acetate in hexane to furnish as a white foam, methyl 6-[5-[1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate (1.678 g, 93%): ESI-MS m/z calc. 704.2281, found 705.5 (M+1)⁺; Retention time: 4.13 minutes (LC Method G).

### Step 4: Methyl 6-[5-[1-Benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate

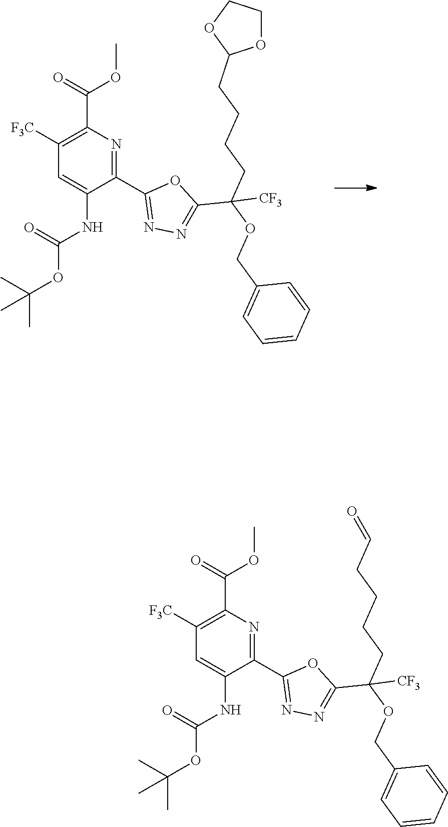 [SMILES: COC(=O)c1nc(-c2nnc(C(CCCCC3OCCO3)(OCc3ccccc3)C(F)(F)F)o2)c(NC(=O)OC(C)(C)C)cc1C(F)(F)F ; COC(=O)c1nc(-c2nnc(C(CCCCC=O)(OCc3ccccc3)C(F)(F)F)o2)c(NC(=O)OC(C)(C)C)cc1C(F)(F)F]

A round bottom flask was charged with methyl 6-[5-[1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate (1.661 g, 2.2041 mmol) in acetic acid (40 mL). Water (10 mL) was added to the reaction mixture. The reaction was stirred at 60° C. for 3 hours. The reaction was cooled to room temperature. Acetic acid was removed under vacuum. The residue was diluted with ethyl acetate (150 mL). The organic solution was washed with saturated sodium bicarbonate (2×50 mL) and brine (50 mL). The ethyl acetate layer was dried over anhydrous sodium sulfate and concentrated under vacuum. The residue was purified by silica gel chromatography using a gradient from 0% to 30% acetone in hexane to furnish as a white foam, methyl 6-[5-[1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate (1.382 g, 89%). ¹H NMR (500 MHz, DMSO-d₆) δ 10.34 (s, 1H), 9.66 (t, J=1.4, 1.4 Hz, 1H), 9.16 (s, 1H), 7.53-7.43 (m, 2H), 7.43-7.27 (m, 3H), 4.71 (d, J=10.6 Hz, 1H), 4.62 (d, J=10.6 Hz, 1H), 3.97 (s, 3H), 2.48-2.32 (m, 2H), 1.68-1.59 (m, 2H), 1.57-1.45 (m, 13H) ppm. ESI-MS m/z calc. 660.20184, found 661.3 (M+1)⁺; Retention time: 3.62 minutes (LC Method H).

### Intermediate 20: Preparation of methyl 6-[5-[(1R)-1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate

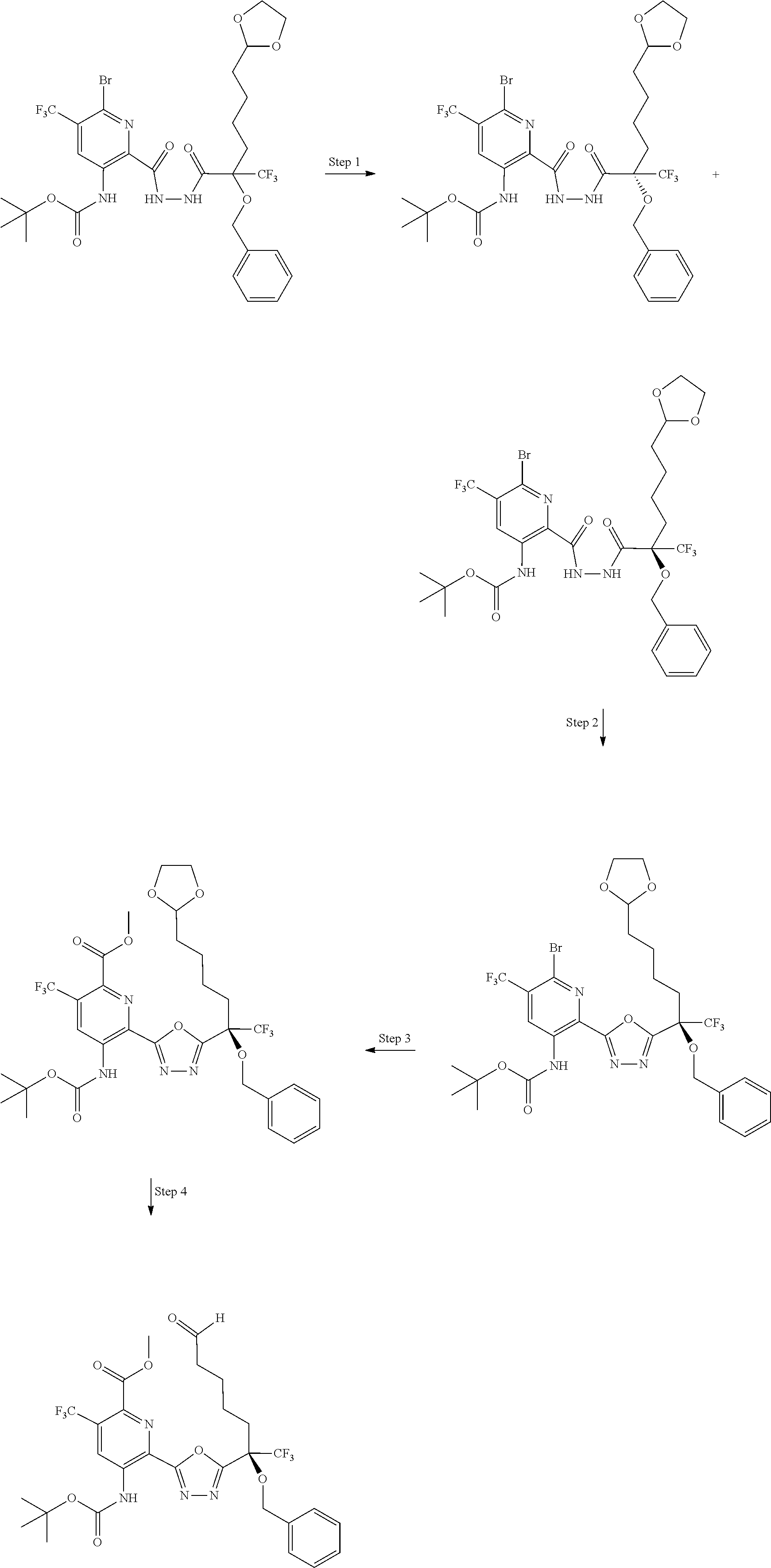

### Step 1: tert-Butyl N-[2-[[[(2R)-2-benzyloxy-6-(1,3-dioxolan-2-yl)-2-(trifluoromethyl)hexanoyl]amino]carbamoyl]-6-bromo-5-(trifluoromethyl)-3-pyridyl]carbamate

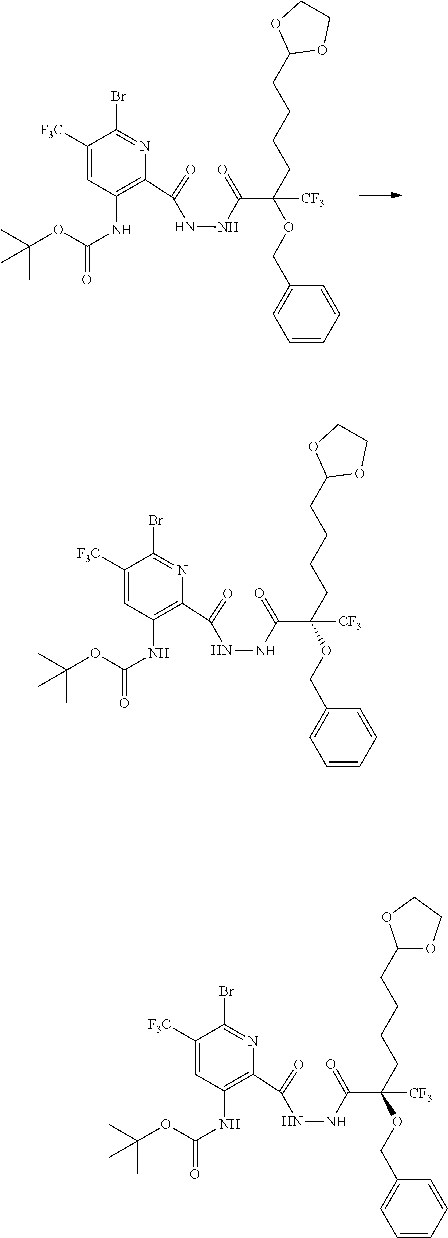 [SMILES: CC(C)(C)OC(=O)Nc1cc(C(F)(F)F)c(Br)nc1C(=O)NNC(=O)C(CCCCC1OCCO1)(OCc1ccccc1)C(F)(F)F ; CC(C)(C)OC(=O)Nc1cc(C(F)(F)F)c(Br)nc1C(=O)NNC(=O)[C@](CCCCC1OCCO1)(OCc1ccccc1)C(F)(F)F ; CC(C)(C)OC(=O)Nc1cc(C(F)(F)F)c(Br)nc1C(=O)NNC(=O)[C@@](CCCCC1OCCO1)(OCc1ccccc1)C(F)(F)F]

The racemic tert-butyl N-[2-[[[2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (16.4 g, 22 mmol) was purified by preparative chiral SFC using a ChiralPak IC column (250×21.2 mm; 5 μm) at 40° C. Mobile phase was 11% MeOH (w/20 mM NH₃), 89% CO₂ at a 70 mL/min flow. Concentration of the sample was 40 mg/mL in methanol (no modifier), injection volume 400 μL with an outlet pressure of 122 bar to provide enantiomer 1, first to elute, tert-butyl N-[2-[[[(2S)-2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (6.77 g, 82%). ¹H NMR (400 MHz, Chloroform-d) δ 10.61 (s, 1H), 9.84 (s, 1H), 9.28 (s, 1H), 9.01 (s, 1H), 7.50-7.30 (m, 5H), 4.93-4.81 (m, 2H), 4.72 (d, J=10.5 Hz, 1H), 4.03-3.90 (m, 2H), 3.90-3.63 (m, 2H), 2.49-2.29 (m, 1H), 2.26-2.10 (m, 1H), 1.71 (p, J=5.7, 4.7 Hz, 2H), 1.63-1.47 (m, 13H) ppm. ¹⁹F NMR (376 MHz, Chloroform-d) δ−63.88, −73.71 ppm. ESI-MS m/z calc. 742.1437, found 645.2 (M+1)⁺; Retention time: 0.44 minutes (LC Method T).

Continued elution provided the second eluting enantiomer 2, tert-butyl N-[2-[[[(2R)-2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (6.83 g, 83%). ESI-MS m/z calc. 742.1437, found 645.2 (M+1)⁺; Retention time: 0.44 minutes (LC Method T).

### Step 2: tert-Butyl N-[2-[5-[(1R)-1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate

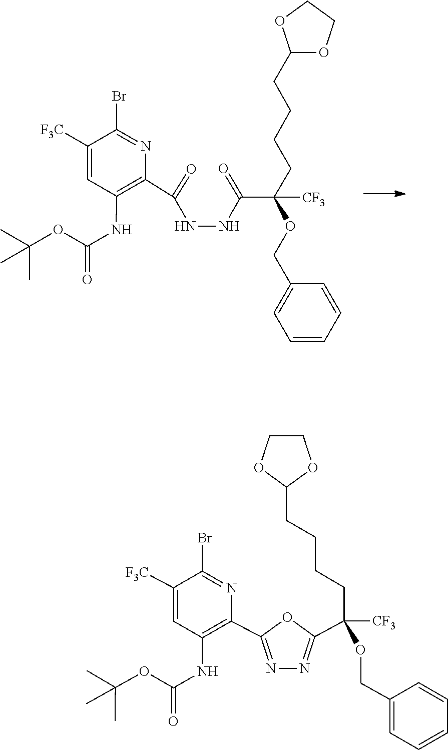 [SMILES: CC(C)(C)OC(=O)Nc1cc(C(F)(F)F)c(Br)nc1C(=O)NNC(=O)[C@@](CCCCC1OCCO1)(OCc1ccccc1)C(F)(F)F ; CC(C)(C)OC(=O)Nc1cc(C(F)(F)F)c(Br)nc1-c1nnc([C@@](CCCCC2OCCO2)(OCc2ccccc2)C(F)(F)F)o1]

To a solution of tert-butyl N-[2-[[[(2R)-2-benzyloxy-6-(1,3-dioxolan-2-yl)-2-(trifluoromethyl)hexanoyl]amino]carbamoyl]-6-bromo-5-(trifluoromethyl)-3-pyridyl]carbamate (enantiomer 2) (2.97 g, 3.995 mmol) in acetonitrile (50 mL) was added p-toluenesulfonyl chloride (936 mg, 4.910 mmol) and DIEA (2 mL, 11.48 mmol). The resulting mixture was heated at 70° C. for 80 min, then diluted with ether (300 mL) and washed with water (500 mL), 1 M NaHCO₃ (2×250 mL), dried (MgSO₄) and evaporated. Purification by silica gel chromatography using a gradient from 0% to 20% ethyl acetate in hexanes provided tert-butyl N-[2-[5-[(1R)-1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate (2.56 g, 88%). ¹H NMR (400 MHz, Chloroform-d) δ 10.19 (s, 1H), 9.34 (s, 1H), 7.49 (d, J=7.5 Hz, 2H), 7.39 (t, J=7.5 Hz, 2H), 7.31 (t, J=7.3 Hz, 1H), 4.86-4.75 (m, 2H), 4.67 (d, J=10.6 Hz, 1H), 4.00-3.90 (m, 2H), 3.84-3.74 (m, 2H), 2.52-2.28 (m, 2H), 1.67 (m, J=7.6, 7.1, 4.7 Hz, 3H), 1.56 (s, 9H), 1.49 (m, J=7.6, 5.8, 3.1 Hz, 3H) ppm. ¹⁹F NMR (376 MHz, Chloroform-d) δ−63.74, −72.67 ppm. ESI-MS m/z calc. 724.1331, found 725.2 (M+1)⁺; Retention time: 0.67 minutes (LC Method T).

### Step 3: Methyl 6-[5-[(1R)-1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate

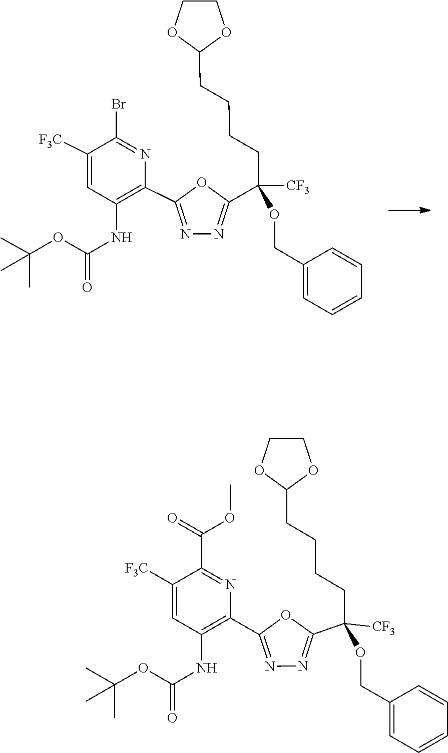 [SMILES: CC(C)(C)OC(=O)Nc1cc(C(F)(F)F)c(Br)nc1-c1nnc([C@@](CCCCC2OCCO2)(OCc2ccccc2)C(F)(F)F)o1 ; COC(=O)c1nc(-c2nnc([C@@](CCCCC3OCCO3)(OCc3ccccc3)C(F)(F)F)o2)c(NC(=O)OC(C)(C)C)cc1C(F)(F)F]

In a stainless steel Parr reaction vessel, a mixture of tert-butyl N-[2-[5-[(1R)-1-benzyloxy-5-(1,3-dioxolan-2-yl)-1-(trifluoromethyl)pentyl]-1,3,4-oxadiazol-2-yl]-6-bromo-5-(trifluoromethyl)-3-pyridyl]carbamate (2.47 g, 3.405 mmol), triethylamine (1.42 mL, 10.19 mmol) and Pd(dppf)Cl₂ (249 mg, 0.3403 mmol) in methanol (25 mL) was degassed by bubbling N₂ for 1 min. The vessel was sealed and put under 100 psi CO, then stirred at 80° C. for 80 min. Then the mixture was cooled to rt, filtered, diluted with ethyl acetate and washed with water, brine, dried (MgSO₄) and evaporated. Purification by silica gel chromatography using a gradient from 5% to 30% ethyl acetate in hexanes provided methyl 6-[5-[(1R)-1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate (2.03 g, 85%). ¹H NMR (400 MHz, Chloroform-d) δ 10.40 (s, 1H), 9.40 (s, 1H), 7.46 (d, J=7.1 Hz, 2H), 7.40-7.34 (m, 2H), 7.33-7.28 (m, 1H), 4.92-4.76 (m, 2H), 4.68 (d, J=10.6 Hz, 1H), 4.02 (s, 3H), 3.96-3.90 (m, 2H), 3.85-3.76 (m, 2H), 2.53-2.30 (m, 2H), 1.67 (m, J=7.7, 7.0, 4.8 Hz, 3H), 1.57 (s, 9H), 1.53-1.41 (m, 3H) ppm. ¹⁹F NMR (376 MHz, Chloroform-d) δ−60.75, −72.62 ppm. ESI-MS m/z calc. 704.2281, found 705.2 (M+1)⁺; Retention time: 0.58 minutes (LC Method T).

### Step 4: Methyl 6-[5-[(1R)-1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate

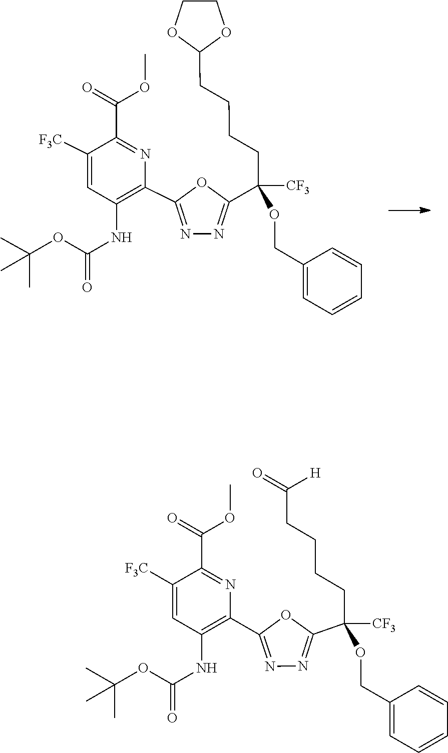 [SMILES: COC(=O)c1nc(-c2nnc([C@@](CCCCC3OCCO3)(OCc3ccccc3)C(F)(F)F)o2)c(NC(=O)OC(C)(C)C)cc1C(F)(F)F ; COC(=O)c1nc(-c2nnc([C@@](CCCCC=O)(OCc3ccccc3)C(F)(F)F)o2)c(NC(=O)OC(C)(C)C)cc1C(F)(F)F]

A mixture of methyl 6-[5-[(1R)-1-benzyloxy-5-(1,3-dioxolan-2-yl)-1-(trifluoromethyl)pentyl]-1,3,4-oxadiazol-2-yl]-5-(tert-butoxycarbonylamino)-3-(trifluoromethyl)pyridine-2-carboxylate (2.03 g, 2.881 mmol), acetic acid (40 mL) and water (10 mL) was stirred at 70° C. for 2 h then cooled to room temperature and diluted with 200 mL ether. The organic solution was washed with water (2×200 mL), 1 M NaHCO₃ (2×150 mL), then dried (MgSO₄) and evaporated. Purification by silica gel chromatography using a gradient from 5% to 40% ethyl acetate in hexanes provided methyl 6-[5-[(1R)-1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate (1.644 g, 78%). ¹H NMR (400 MHz, Chloroform-d) δ 10.38 (s, 1H), 9.74 (d, J=1.3 Hz, 1H), 9.41 (s, 1H), 7.45 (d, J=7.5 Hz, 2H), 7.42-7.28 (m, 3H), 4.81 (d, J=10.6 Hz, 1H), 4.69 (d, J=10.6 Hz, 1H), 4.02 (s, 3H), 2.50-2.30 (m, 4H), 1.77-1.61 (m, 2H), 1.58 (s, 9H), 1.52-1.40 (m, 2H) ppm. ¹⁹F NMR (376 MHz, Chloroform-d) δ−60.77, −72.56 ppm. ESI-MS m/z calc. 660.20184, found 661.2 (M+1)⁺; Retention time: 0.43 minutes (LC Method T).

### Intermediate 21: Preparation of methyl 6-[5-[1-benzyloxy-5-oxo-1-(trifluoromethyl)pentyl]-1,3,4-oxadiazol-2-yl]-5-(tert-butoxycarbonylamino)-3-(trifluoromethyl)pyridine-2-carboxylate

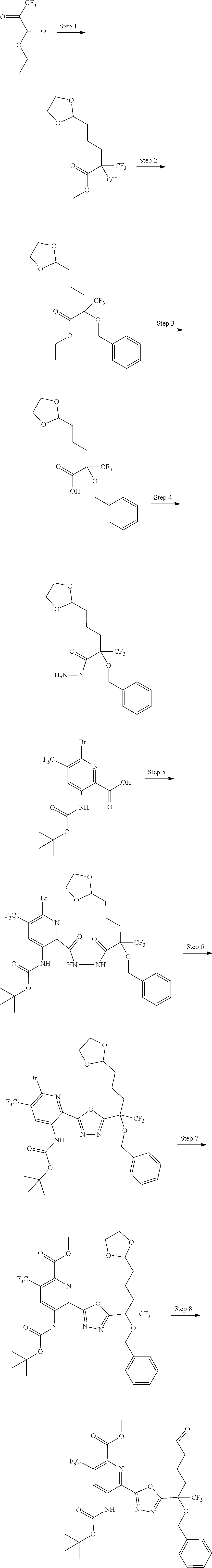

### Step 1: Ethyl 5-(1,3-Dioxolan-2-yl)-2-hydroxy-2-(trifluoromethyl)pentanoate

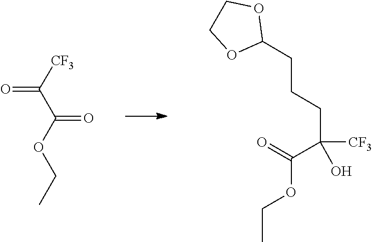 [SMILES: CCOC(=O)C(=O)C(F)(F)F ; CCOC(=O)C(O)(CCCC1OCCO1)C(F)(F)F]

To a solution of ethyl 3,3,3-trifluoro-2-oxo-propanoate (7.6980 g, 6 mL, 45.259 mmol) in diethyl ether (75 mL) at −78° C. was added chloro-[3-(1,3-dioxolan-2-yl)propyl]magnesium (52 mL of 0.92 M, 47.84 mmol) dropwise over a period of 20 minutes. The mixture was stirred at −78° C. for 20 min. The dry ice-acetone bath was removed. The reaction mixture was warmed slowly to 10° C. for 20 minutes, then added to a mixture of 1N aqueous hydrochloric acid (200 mL) and crushed ice (50 g). The two layers were separated, and the residue aqueous phase was extracted with ethyl acetate (3×100 mL). The organic layers were combined, washed with aqueous saturated sodium bicarbonate solution (100 mL) and brine (100 mL) and dried over sodium sulfate. The mixture was filtered and concentrated to provide as a colorless oil, ethyl 5-(1,3-dioxolan-2-yl)-2-hydroxy-2-(trifluoromethyl)pentanoate (13.9 g, 97%). ¹H NMR (400 MHz, CDCl₃) δ 4.90-4.82 (m, 1H), 4.46-4.30 (m, 2H), 4.01-3.92 (m, 2H), 3.90-3.80 (m, 3H), 2.12-2.02 (m, 1H), 1.99-1.86 (m, 1H), 1.77-1.58 (m, 3H), 1.39-1.32 (m, 3H), 1.31-1.23 (m, 1H) ppm. ¹⁹F NMR (377 MHz, CDCl₃) δ−78.65 (s, 3F) ppm.

### Step 2: Ethyl 2-benzyloxy-5-(1,3-dioxolan-2-yl)-2-(trifluoromethyl)pentanoate

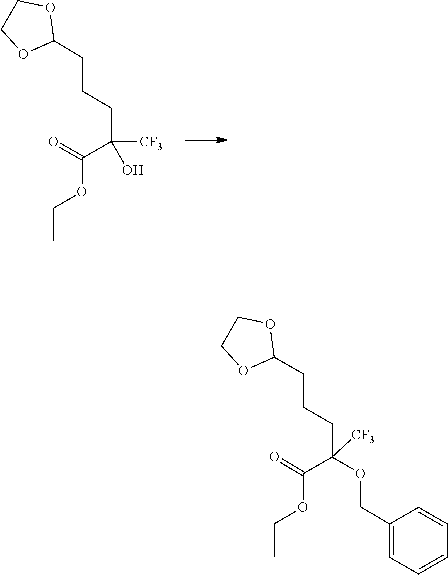 [SMILES: CCOC(=O)C(O)(CCCC1OCCO1)C(F)(F)F ; CCOC(=O)C(CCCC1OCCO1)(OCc1ccccc1)C(F)(F)F]

To a solution of ethyl 5-(1,3-dioxolan-2-yl)-2-hydroxy-2-(trifluoromethyl)pentanoate (1.7 g, 4.9293 mmol) in DMF (7 mL) at 0° C. was added NaH (352 mg, 8.8008 mmol) in portions. The mixture was stirred at 0° C. for 30 min. Bromoethylbenzene (1.5818 g, 1.1 mL, 9.2485 mmol) was added dropwise, followed by tetrabutylammonium iodide (318 mg, 0.8609 mmol). The mixture was slowly warmed to room temperature and stirred at room temperature overnight. Ammonium chloride (218 mg, 4.0754 mmol) was added. The mixture was stirred at room temperature for 10 min. Water (200 mL) was added, followed by a mixture of MTBE and heptanes (2:1, 100 mL). The two layers were separated, and the aqueous phase was extracted with a mixture of MTBE and heptane (2:1, 2×100 mL). The combined organic layers were washed with water (100 mL) and brine (100 mL) than dried over sodium sulfate. The mixture was filtered and concentrated under reduced pressure. The residue was purified by silica gel chromatography using 0% to 30% ethyl acetate in heptanes to afford as a colorless oil, ethyl 2-benzyloxy-5-(1,3-dioxolan-2-yl)-2-(trifluoromethyl)pentanoate (1.6 g, 78%). ¹H NMR (400 MHz, CDCl₃) δ 7.48-7.30 (m, 5H), 4.89-4.79 (m, 2H), 4.72-4.61 (m, 1H), 4.42-4.29 (m, 2H), 4.01-3.93 (m, 2H), 3.89-3.80 (m, 2H), 2.15-1.99 (m, 2H), 1.77-1.63 (m, 3H), 1.56-1.46 (m, 1H), 1.36 (t, J=7.2 Hz, 3H) ppm. ¹⁹F NMR (377 MHz, CDCl₃) δ−70.46 (s, 3F) ppm. ESI-MS m/z calc. 376.1498, found 399.2 (M+23)⁺; Retention time: 1.98 minutes (LC Method Z).

### Step 3: 2-Benzyloxy-5-(1,3-dioxolan-2-yl)-2-(trifluoromethyl)pentanoic Acid

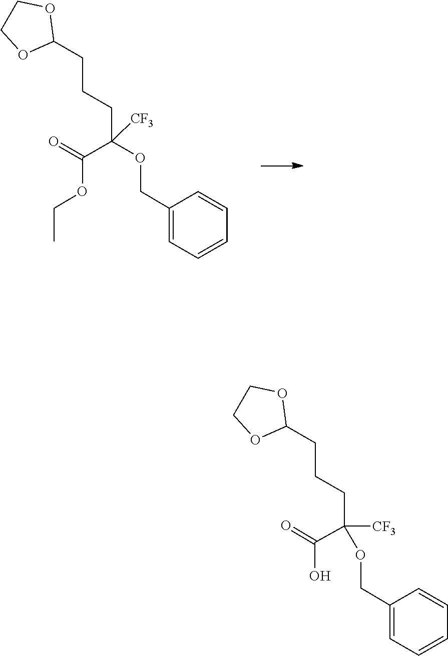 [SMILES: CCOC(=O)C(CCCC1OCCO1)(OCc1ccccc1)C(F)(F)F ; O=C(O)C(CCCC1OCCO1)(OCc1ccccc1)C(F)(F)F]

A solution of sodium hydroxide (12.2 g, 305.02 mmol) in water (120 mL) was added to a solution of ethyl 2-benzyloxy-5-(1,3-dioxolan-2-yl)-2-(trifluoromethyl)pentanoate (45.2 g, 120.10 mmol) in methanol (360 mL). The reaction was heated at 50° C. overnight. The reaction was concentrated to removed methanol. The crude material was diluted in water (500 mL) and the carboxylate sodium salt was washed with heptane (200 mL) and MTBE (200 mL). The aqueous solution was acidified to pH=2 with 3N aqueous hydrochloric acid solution. The carboxylic acid was extracted with dichloromethane (4×200 mL) and dried over sodium sulfate. The solution was filtered and concentrated to provide as a yellow oil, 2-benzyloxy-5-(1,3-dioxolan-2-yl)-2-(trifluoromethyl)pentanoic acid (43.2 g, quant.). ¹H NMR (400 MHz, CDCl₃) δ 8.94 (br s, 1H), 7.51-7.29 (m, 5H), 4.94-4.80 (m, 2H), 4.70 (d, J=10.4 Hz, 1H), 4.04-3.92 (m, 2H), 3.90-3.77 (m, 2H), 2.29-2.08 (m, 2H), 1.80-1.64 (m, 3H), 1.62-1.46 (m, 1H) ppm. ¹⁹F NMR (377 MHz, CDCl₃) δ−71.58 (s, 3F) ppm. ESI-MS m/z calc. 348.1185, found 347.2 (M−1); Retention time: 4.19 minutes (LC Method AA).

### Step 4: 2-Benzyloxy-5-(1,3-dioxolan-2-yl)-2-(trifluoromethyl)pentanehydrazide

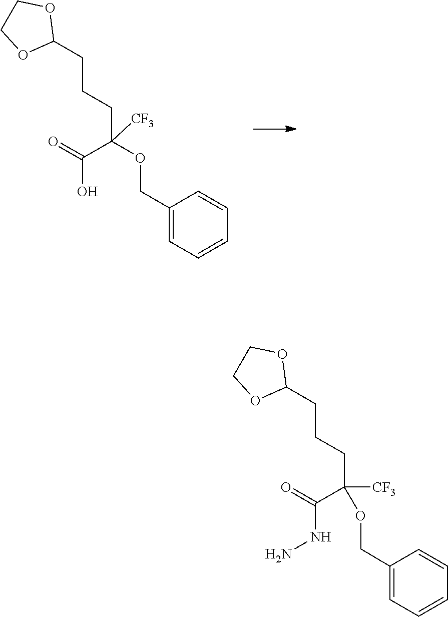 [SMILES: O=C(O)C(CCCC1OCCO1)(OCc1ccccc1)C(F)(F)F ; NNC(=O)C(CCCC1OCCO1)(OCc1ccccc1)C(F)(F)F]

To a solution of 2-benzyloxy-5-(1,3-dioxolan-2-yl)-2-(trifluoromethyl)pentanoic acid (6.004 g, 16.375 mmol) in DMF (60 mL) was added HATU (8.735 g, 22.973 mmol) and triethylamine (3.6300 g, 5 mL, 35.873 mmol). The mixture was stirred for 10 minutes. Hydrazine hydrate (16.512 g, 16 mL, 329.84 mmol) was added to the reaction mixture. The reaction was stirred at room temperature for 1.5 hours. The reaction was diluted with water (200 mL) and ethyl acetate (200 mL). Two layers were separated, and the aqueous layer was extracted with ethyl acetate (2×100 mL). The combined organic layers were washed with brine (3×100 mL), dried over anhydrous magnesium sulfate and concentrated under vacuum. The residue was purified by silica gel chromatography using a gradient from 0% to 10% methanol in DCM to furnish as an orange oil, 2-benzyloxy-5-(1,3-dioxolan-2-yl)-2-(trifluoromethyl)pentanehydrazide (6.075 g, 97%). ESI-MS m/z calc. 362.1453, found 363.3 (M+1)⁺; Retention time: 2.33 minutes (LC Method G).

### Step 5: tert-Butyl N-[2-[[[2-benzyloxy-5-(1,3-dioxolan-2-yl)-2-(trifluoromethyl)pentanoyl]amino]carbamoyl]-6-bromo-5-(trifluoromethyl)-3-pyridyl]carbamate

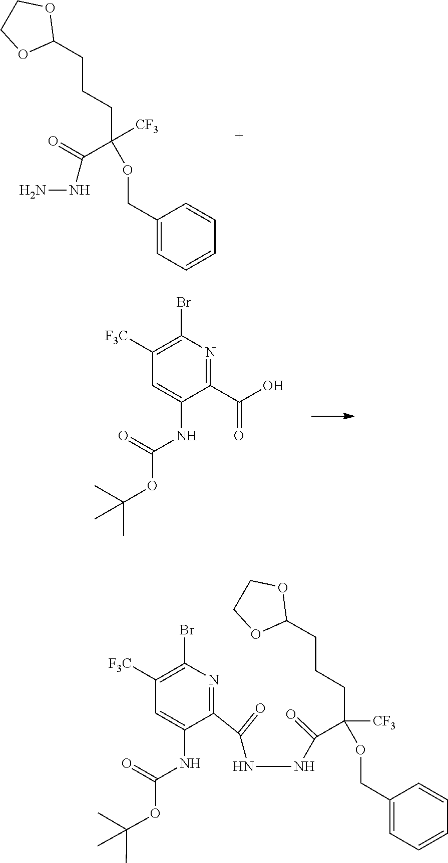

To a mixture of 6-bromo-3-(tert-butoxycarbonylamino)-5-(trifluoromethyl)pyridine-2-carboxylic acid (9.62 g, 24.98 mmol) and 2-benzyloxy-5-(1,3-dioxolan-2-yl)-2-(trifluoromethyl)pentanehydrazide (10 g, 27.60 mmol) in ethyl acetate (100 mL) at ambient temperature was added pyridine (8.2 mL, 101.4 mmol) which afforded a precipitate. To the mixture was added T3P (20.6 mL of 50% w/v, 32.37 mmol) and the reaction mixture stirred at ambient temperature for 5 h. The reaction was quenched with the slow addition of NaOH (50% in water) (6.3 mL of 50% w/v, 78.76 mmol) in water (100 mL) and the mixture stirred for 1 h. The organic phase was separated, and the aqueous phase extracted with ethyl acetate (100 mL). The combined organic phases washed with HCl (40 mL of 1 M), brine, dried (MgSO₄), filtered and evaporated. The residue was purified by silica gel chromatography using 5% to 30% ethyl acetate in hexanes over 20 min to provide tert-butyl N-[2-[[[2-benzyloxy-5-(1,3-dioxolan-2-yl)-2-(trifluoromethyl)pentanoyl]amino]carbamoyl]-6-bromo-5-(trifluoromethyl)-3-pyridyl]carbamate (10.92 g, 60%). ¹H NMR (400 MHz, Chloroform-d) δ 10.60 (s, 1H), 9.84 (s, 1H), 9.28 (s, 1H), 8.99 (s, 1H), 7.47-7.33 (m, 5H), 4.90 (t, J=4.5 Hz, 1H), 4.84 (d, J=10.4 Hz, 1H), 4.75 (d, J=10.5 Hz, 1H), 4.04-3.92 (m, 2H), 3.88-3.79 (m, 2H), 2.39 (m, J=16.6, 11.2, 5.7 Hz, 1H), 2.28 (m, J=14.9, 11.2, 4.9 Hz, 1H), 1.91-1.58 (m, 4H), 1.52 (s, 9H) ppm. ¹⁹F NMR (376 MHz, Chloroform-d) δ−63.88, −73.65 ppm. ESI-MS m/z calc. 728.12805, found 631.0 (M+1)⁺; Retention time: 0.41 minutes (LC Method T).

### Step 6: tert-Butyl N-[2-[5-[1-benzyloxy-4-(1,3-dioxolan-2-yl)-1-(trifluoromethyl)butyl]-1,3,4-oxadiazol-2-yl]-6-bromo-5-(trifluoromethyl)-3-pyridyl]carbamate

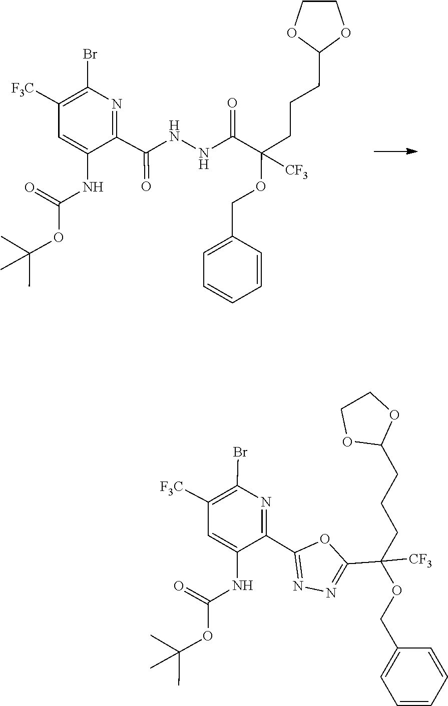 [SMILES: CC(C)(C)OC(=O)Nc1cc(C(F)(F)F)c(Br)nc1C(=O)NNC(=O)C(CCCC1OCCO1)(OCc1ccccc1)C(F)(F)F ; CC(C)(C)OC(=O)Nc1cc(C(F)(F)F)c(Br)nc1-c1nnc(C(CCCC2OCCO2)(OCc2ccccc2)C(F)(F)F)o1]

To a solution of tert-butyl N-[2-[[[2-benzyloxy-5-(1,3-dioxolan-2-yl)-2-(trifluoromethyl)pentanoyl]amino]carbamoyl]-6-bromo-5-(trifluoromethyl)-3-pyridyl]carbamate (10.92 g, 14.97 mmol) in acetonitrile (200 mL) was added p-toluenesulfonyl chloride (3.5 g, 18.36 mmol) and DIEA (7.5 mL, 43.06 mmol). The resulting mixture was heated at 70° C. for 80 min, then diluted with ether (300 mL) and washed with water (500 mL), 1 M NaHCO₃ (2×300 mL), dried (MgSO₄) and evaporated. The residue was purified by silica gel chromatography using 0% to 20% ethyl acetate in hexanes over 15 min to provide tert-butyl N-[2-[5-[1-benzyloxy-4-(1,3-dioxolan-2-yl)-1-(trifluoromethyl)butyl]-1,3,4-oxadiazol-2-yl]-6-bromo-5-(trifluoromethyl)-3-pyridyl]carbamate (9.67 g, 91%). ¹H NMR (400 MHz, Chloroform-d) δ 10.19 (s, 1H), 9.34 (s, 1H), 7.49 (d, J=7.5 Hz, 2H), 7.39 (t, J=7.5 Hz, 2H), 7.31 (t, J=7.4 Hz, 1H), 4.86 (t, J=4.1 Hz, 1H), 4.80 (d, J=10.6 Hz, 1H), 4.68 (d, J=10.6 Hz, 1H), 3.99-3.89 (m, 2H), 3.87-3.73 (m, 2H), 2.71-2.34 (m, 2H), 1.73 (m, 4H), 1.56 (s, 9H) ppm. ¹⁹F NMR (376 MHz, Chloroform-d) δ−63.74, −72.61 ppm. ESI-MS m/z calc. 710.1175, found 713.2 (M+1)⁺; Retention time: 0.52 minutes (LC Method T).

### Step 7: Methyl 6-[5-[1-benzyloxy-4-(1,3-dioxolan-2-yl)-1-(trifluoromethyl)butyl]-1,3,4-oxadiazol-2-yl]-5-(tert-butoxycarbonylamino)-3-(trifluoromethyl)pyridine-2-carboxylate

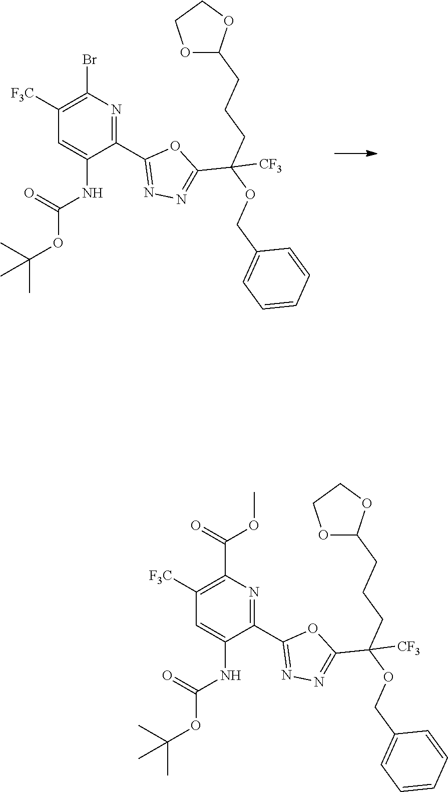 [SMILES: CC(C)(C)OC(=O)Nc1cc(C(F)(F)F)c(Br)nc1-c1nnc(C(CCCC2OCCO2)(OCc2ccccc2)C(F)(F)F)o1 ; COC(=O)c1nc(-c2nnc(C(CCCC3OCCO3)(OCc3ccccc3)C(F)(F)F)o2)c(NC(=O)OC(C)(C)C)cc1C(F)(F)F]

In a 450 mL stainless steel Parr reaction vessel, a mixture of tert-butyl N-[2-[5-[1-benzyloxy-4-(1,3-dioxolan-2-yl)-1-(trifluoromethyl)butyl]-1,3,4-oxadiazol-2-yl]-6-bromo-5-(trifluoromethyl)-3-pyridyl]carbamate (9.67 g, 13.59 mmol), triethylamine (6 mL, 43.05 mmol) and PdCl₂(dppf) (500 mg, 0.6833 mmol) in methanol (70 mL) and ethyl acetate (30 mL) was degassed by bubbling N₂ for 1 min. The vessel was sealed and put under 100 psi CO, then stirred at 80° C. for 1 h. The mixture was cooled to room temperature and PdCl₂(dppf) (500 mg, 0.6833 mmol) added, put under 100 psi CO, then stirred at 80° C. for 3 h. Then the mixture was cooled to room temperature, filtered, diluted with ethyl acetate and washed with water, brine, dried (MgSO₄) and evaporated. The residue was purified by silica gel chromatography using 5% to 30% ethyl acetate in hexanes over 15 min to provide methyl 6-[5-[1-benzyloxy-4-(1,3-dioxolan-2-yl)-1-(trifluoromethyl)butyl]-1,3,4-oxadiazol-2-yl]-5-(tert-butoxycarbonylamino)-3-(trifluoromethyl)pyridine-2-carboxylate (8.26 g, 88%). ¹H NMR (400 MHz, Chloroform-d) δ 10.40 (s, 1H), 9.40 (s, 1H), 7.50-7.43 (m, 2H), 7.41-7.34 (m, 2H), 7.31 (d, J=7.3 Hz, 1H), 4.85 (t, J=4.2 Hz, 1H), 4.82 (d, J=10.6 Hz, 1H), 4.68 (d, J=10.6 Hz, 1H), 4.01 (s, 3H), 3.98-3.88 (m, 2H), 3.87-3.76 (m, 2H), 2.56-2.39 (m, 2H), 1.83-1.68 (m, 4H), 1.57 (s, 9H) ppm. ¹⁹F NMR (376 MHz, Chloroform-d) δ−60.76, −72.56 ppm. ESI-MS m/z calc. 690.21246, found 691.3 (M+1)⁺; Retention time: 0.44 minutes (LC Method T).

### Step 8: Methyl 6-[5-[1-benzyloxy-5-oxo-1-(trifluoromethyl)pentyl]-1,3,4-oxadiazol-2-yl]-5-(tert-butoxycarbonylamino)-3-(trifluoromethyl)pyridine-2-carboxylate

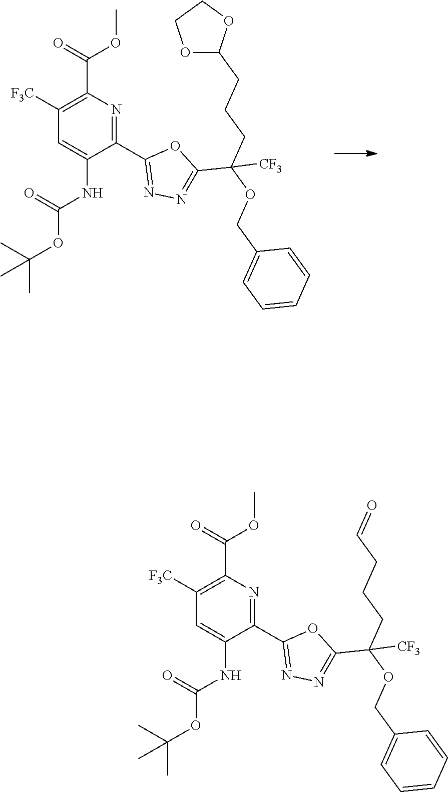 [SMILES: COC(=O)c1nc(-c2nnc(C(CCCC3OCCO3)(OCc3ccccc3)C(F)(F)F)o2)c(NC(=O)OC(C)(C)C)cc1C(F)(F)F ; COC(=O)c1nc(-c2nnc(C(CCCC=O)(OCc3ccccc3)C(F)(F)F)o2)c(NC(=O)OC(C)(C)C)cc1C(F)(F)F]

A round bottom flask was charged with methyl 6-[5-[1-benzyloxy-4-(1,3-dioxolan-2-yl)-1-(trifluoromethyl)butyl]-1,3,4-oxadiazol-2-yl]-5-(tert-butoxycarbonylamino)-3-(trifluoromethyl)pyridine-2-carboxylate (2.308 g, 3.3421 mmol) in acetic acid (55 mL). Water (14 mL) was added to the reaction mixture. The reaction was stirred at 60° C. for 3 hours. The reaction was cooled to room temperature. Acetic acid was removed under vacuum. The residue was diluted with ethyl acetate (100 mL). The organic solution was washed with saturated sodium bicarbonate (2×20 mL) and brine (20 mL). The ethyl acetate layer was dried over anhydrous sodium sulfate and concentrated under vacuum. The residue was purified by silica gel chromatography using a gradient from 0% to 20% ethyl acetate in hexanes to furnish as a white foam, methyl 6-[5-[1-benzyloxy-5-oxo-1-(trifluoromethyl)pentyl]-1,3,4-oxadiazol-2-yl]-5-(tert-butoxycarbonylamino)-3-(trifluoromethyl)pyridine-2-carboxylate (1.564 g, 72%). ESI-MS m/z calc. 646.1862, found 647.4 (M+1)⁺; Retention time: 3.94 minutes (LC Method G).

### Intermediate 22: Preparation of methyl 5-(tert-butoxycarbonylamino)-6-[5-[2,2,2-trifluoro-1-[2-fluoro-5-(3-oxopropyl)phenyl]-1-hydroxy-ethyl]-1,3,4-oxadiazol-2-yl]-3-(trifluoromethyl)pyridine-2-carboxylate

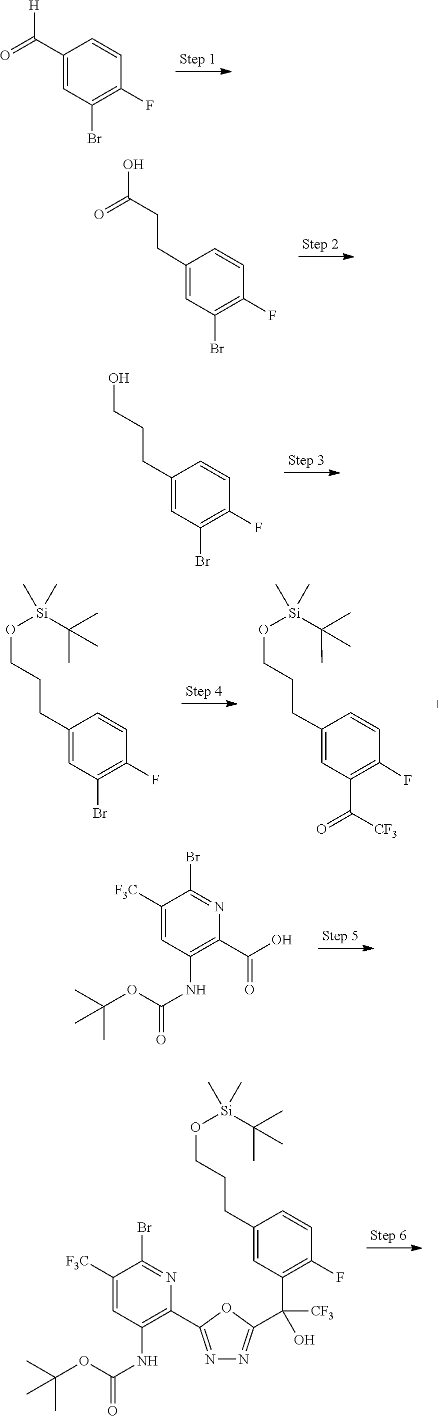

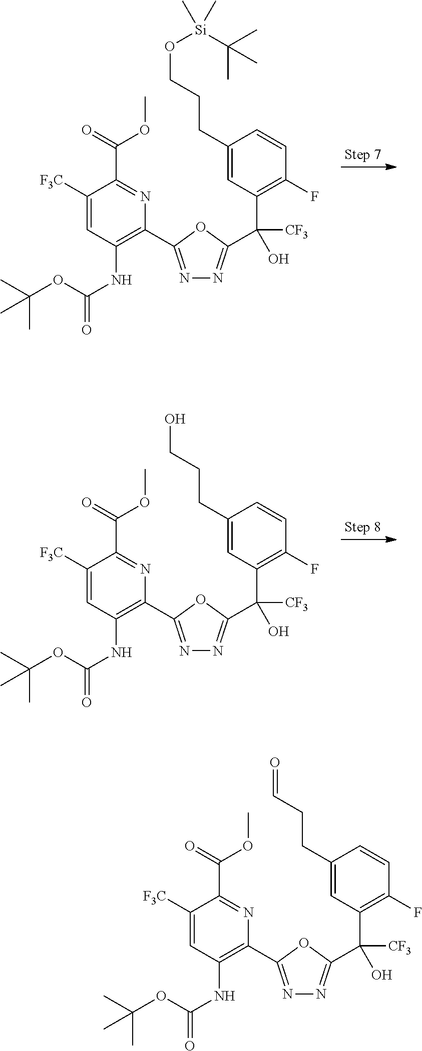 [SMILES: COC(=O)c1nc(-c2nnc(C(O)(c3cc(CCCO[Si](C)(C)C(C)(C)C)ccc3F)C(F)(F)F)o2)c(NC(=O)OC(C)(C)C)cc1C(F)(F)F ; COC(=O)c1nc(-c2nnc(C(O)(c3cc(CCCO)ccc3F)C(F)(F)F)o2)c(NC(=O)OC(C)(C)C)cc1C(F)(F)F ; COC(=O)c1nc(-c2nnc(C(O)(c3cc(CCC=O)ccc3F)C(F)(F)F)o2)c(NC(=O)OC(C)(C)C)cc1C(F)(F)F]

### Step 1: 3-(3-Bromo-4-fluoro-phenyl)propanoic Acid

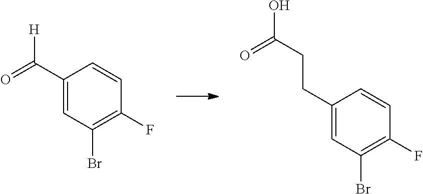 [SMILES: O=Cc1ccc(F)c(Br)c1 ; O=C(O)CCc1ccc(F)c(Br)c1]

Triethylamine (16.6 mL) was placed in a round bottom flask and briefly chilled with ice water bath. Formic acid (11.2 mL) was added in small portions. The mixture was stirred at room temperature for 25 minutes and DMF (75 mL) was added. 3-Bromo-4-fluoro-benzaldehyde (20 g, 98.518 mmol) was added, followed by 2,2-dimethyl-1,3-dioxane-4,6-dione (14.2 g, 98.526 mmol). The mixture was heated at 100° C. for 16 h. It was then cooled to room temperature and poured into ice water (500 mL). The mixture was extracted with dichloromethane (2×300 mL). The combined organic layer was extracted with 10% aqueous NaOH (2×200 mL). The combined basic aqueous solution was acidified with concentrated HCl to pH=2. The resulted mixture was then extracted with ethyl acetate (3×150 mL). The combined organic layer washed with water (200 mL), brine (200 mL), dried over anhydrous. Sodium sulfate, filtered and concentrated to afford as white solid, 3-(3-bromo-4-fluoro-phenyl)propanoic acid (23.2 g, 86%). ESI-MS m/z calc. 245.9692, found 247.3 (M+1)⁺; Retention time: 3.09 minutes (LC Method G).

### Step 2: 3-(3-Bromo-4-fluoro-phenyl)propan-1-ol

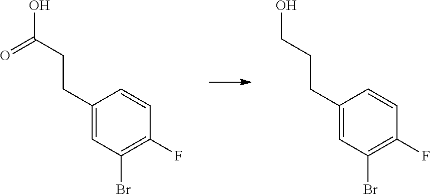 [SMILES: O=C(O)CCc1ccc(F)c(Br)c1 ; OCCCc1ccc(F)c(Br)c1]

3-(3-Bromo-4-fluoro-phenyl)propanoic acid (23.2 g, 84.514 mmol) was dissolved in THF (150 mL) and the solution was cooled in ice water bath. Borane dimethylsulfide complex (28 mL of 10 M, 280 mmol) was added drop wise. The mixture was then heated at reflux for 20 h. It was cooled to room temperature and placed in ice water bath. MeOH was added drop wise to quench all unreacted borane and concentrated under vacuum. The residue was dissolved in ethyl acetate (200 mL) and washed with aqueous HCl (80 mL, 3N), aqueous NaOH (80 mL, 3N), brine, dried over anhydrous magnesium sulfate, filtered and concentrated to afford as a colorless oil, 3-(3-bromo-4-fluoro-phenyl)propan-1-ol (21.3 g, 97%). ¹H NMR (500 MHz, Chloroform-d) δ 7.39 (dd, J=6.6, 2.3 Hz, 1H), 7.15-7.06 (m, 1H), 7.06-6.96 (m, 1H), 3.66 (t, J=6.6 Hz, 2H), 2.73-2.60 (m, 2H), 1.91-1.80 (m, 2H), 1.32 (s, 1H) ppm. ESI-MS m/z calc. 231.9899, found 233.2 (M+1)⁺; Retention time: 3.16 minutes (LC Method G).

### Step 3: 3-(3-Bromo-4-fluoro-phenyl)propoxy-tert-butyl-dimethyl-silane

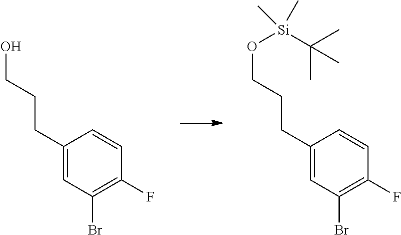 [SMILES: OCCCc1ccc(F)c(Br)c1 ; CC(C)(C)[Si](C)(C)OCCCc1ccc(F)c(Br)c1]

To a solution of 3-(3-bromo-4-fluoro-phenyl)propan-1-ol (10 g, 38.614 mmol) in DMF (100 mL) at room temperature was added imidazole (3.2 g, 47.005 mmol), followed portion-wise by tert-butyl-chloro-dimethyl-silane (6.8 g, 45.116 mmol). The mixture was stirred at room temperature for 2 h and diluted with ethyl acetate (200 mL), followed by water (300 mL). The organic layer was separated and washed with water (3×100 mL), brine, dried over anhydrous magnesium sulfate, filtered and concentrated. The residue was purified by silica gel chromatography, using 0% to 20% ethyl acetate in hexanes to afford as white solid, 3-(3-bromo-4-fluoro-phenyl)propoxy-tert-butyl-dimethyl-silane (12.52 g, 89%). ¹H NMR (500 MHz, Chloroform-d) δ 7.38 (dd, J=6.6, 2.2 Hz, 1H), 7.08 (m, 1H), 7.02 (t, J=8.4, 8.4 Hz, 1H), 3.61 (t, J=6.1, 6.1 Hz, 2H), 2.69-2.60 (m, 2H), 1.86-1.73 (m, 2H), 0.91 (s, 9H), 0.05 (s, 6H) ppm. ESI-MS m/z calc. 346.0764, found 347.3 (M+1)⁺; Retention time: 5.08 minutes (LC Method G).

### Step 4: 1-[5-[3-[tert-Butyl(dimethyl)silyl]oxypropyl]-2-fluoro-phenyl]-2,2,2-trifluoro-ethanone

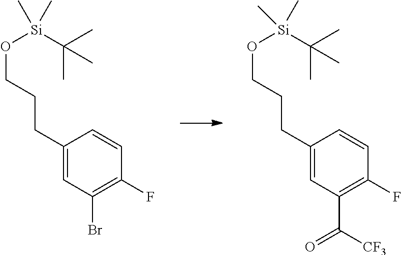 [SMILES: CC(C)(C)[Si](C)(C)OCCCc1ccc(F)c(Br)c1 ; CC(C)(C)[Si](C)(C)OCCCc1ccc(F)c(C(=O)C(F)(F)F)c1]

To a cooled (dry ice-acetone bath) solution of 3-(3-bromo-4-fluoro-phenyl)propoxy-tert-butyl-dimethyl-silane (9.4 g, 25.710 mmol) in THF (120 mL) was added n-BuLi in hexanes (11.5 mL of 2.5 M, 28.750 mmol) and stirred under N₂ balloon for 25 min. Ethyl 2,2,2-trifluoroacetate (7.1460 g, 6 mL, 50.297 mmol) in THF (10 mL) was then added drop wise over 5 min. The mixture was stirred at this temperature for 15 min and with cooling bath removed. Water (˜40 mL) was added and extracted with ethyl acetate (60 mL). The organic layer was washed with brine, dried over anhydrous MgSO₄, filtered and concentrated. The residue was purified by silica gel chromatography, using 0% to 20% ethyl acetate in hexanes to afford as a white solid, 1-[5-[3-[tert-butyl(dimethyl)silyl]oxypropyl]-2-fluoro-phenyl]-2,2,2-trifluoro-ethanone (7.6 g, 73%). ESI-MS m/z calc. 364.1482, found 365.5 (M+1)⁺; Retention time: 4.96 minutes (LC Method G).

### Step 5: tert-Butyl N-[6-bromo-2-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-5-(trifluoromethyl)-3-pyridyl]carbamate

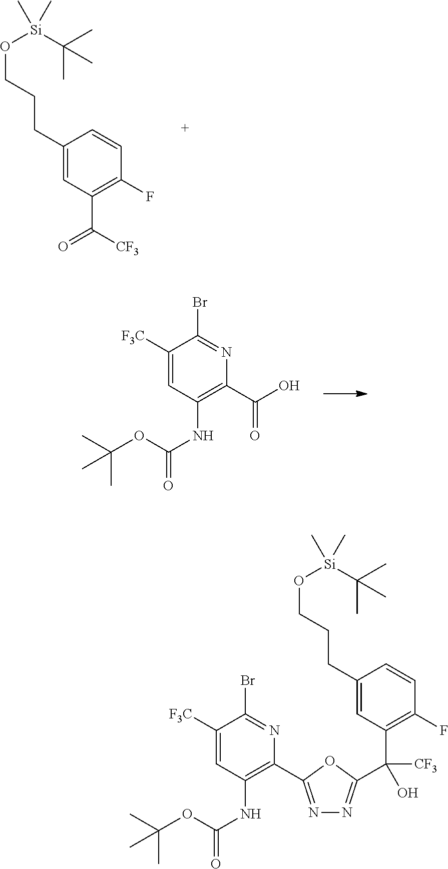 [SMILES: CC(C)(C)[Si](C)(C)OCCCc1ccc(F)c(C(=O)C(F)(F)F)c1 ; CC(C)(C)OC(=O)Nc1cc(C(F)(F)F)c(Br)nc1C(=O)O ; CC(C)(C)OC(=O)Nc1cc(C(F)(F)F)c(Br)nc1-c1nnc(C(O)(c2cc(CCCO[Si](C)(C)C(C)(C)C)ccc2F)C(F)(F)F)o1]

To a solution of 1-[5-[3-[tert-butyl(dimethyl)silyl]oxypropyl]-2-fluoro-phenyl]-2,2,2-trifluoro-ethanone (1.38 g, 3.4079 mmol) in DCM (8 mL) at room temperature was added (N-isocyanoimino)triphenylphosphorane (1.03 g, 3.4071 mmol) in DCM (8 mL) drop wise over 10 min. The brown solution was stirred at room temperature overnight and concentrated. The residue was purified by silica gel chromatography using 0% to 15% ethyl acetate in hexanes to afford as a white solid, tert-butyl N-[6-bromo-2-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-5-(trifluoromethyl)-3-pyridyl]carbamate (870 mg, 47%). ESI-MS m/z calc. 772.1527, found 673.4 (M−100)+; Retention time: 5.22 minutes (LC Method G).

### Step 6: Methyl 5-(tert-butoxycarbonylamino)-6-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-3-(trifluoromethyl)pyridine-2-carboxylate

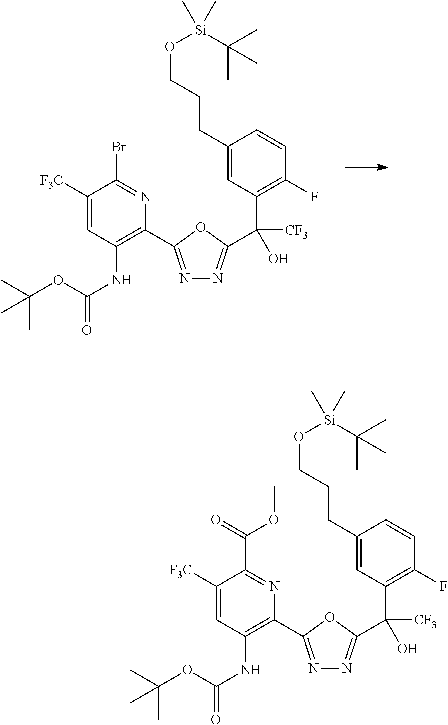 [SMILES: CC(C)(C)OC(=O)Nc1cc(C(F)(F)F)c(Br)nc1-c1nnc(C(O)(c2cc(CCCO[Si](C)(C)C(C)(C)C)ccc2F)C(F)(F)F)o1 ; COC(=O)c1nc(-c2nnc(C(O)(c3cc(CCCO[Si](C)(C)C(C)(C)C)ccc3F)C(F)(F)F)o2)c(NC(=O)OC(C)(C)C)cc1C(F)(F)F]

To a solution of tert-butyl N-[6-bromo-2-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-5-(trifluoromethyl)-3-pyridyl]carbamate (3.2 g, 3.7228 mmol) in MeOH (50 mL) at room temperature was added TEA (1.0890 g, 1.5 mL, 10.762 mmol), followed by Pd(dppf)Cl₂ (150 mg, 0.2050 mmol). The mixture was purged with N₂ for 15 min. It was then sealed and subject to ˜100 psi CO at 80° C. temperature for 6 h. After cooling to rt, the mixture was transferred to a flask and concentrated. The residue was purified by silica gel chromatography, using 0% to 15% ethyl acetate in hexanes to afford as a white solid, methyl 5-(tert-butoxycarbonylamino)-6-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-3-(trifluoromethyl)pyridine-2-carboxylate (1.8 g, 58%). ESI-MS m/z calc. 752.2476, found 753.6 (M+1)⁺; Retention time: 5.07 minutes (LC Method G).

### Step 7: Methyl 5-(tert-butoxycarbonylamino)-6-[5-[2,2,2-trifluoro-1-[2-fluoro-5-(3-hydroxypropyl)phenyl]-1-hydroxy-ethyl]-1,3,4-oxadiazol-2-yl]-3-(trifluoromethyl)pyridine-2-carboxylate

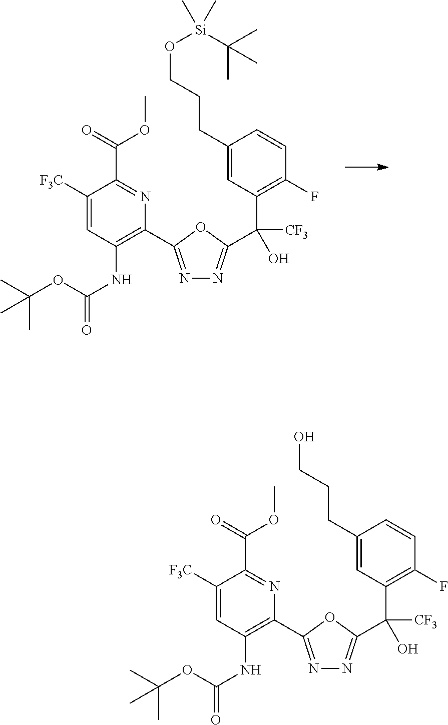 [SMILES: COC(=O)c1nc(-c2nnc(C(O)(c3cc(CCCO[Si](C)(C)C(C)(C)C)ccc3F)C(F)(F)F)o2)c(NC(=O)OC(C)(C)C)cc1C(F)(F)F ; COC(=O)c1nc(-c2nnc(C(O)(c3cc(CCCO)ccc3F)C(F)(F)F)o2)c(NC(=O)OC(C)(C)C)cc1C(F)(F)F]

To a solution of methyl 5-(tert-butoxycarbonylamino)-6-[5-[1-[5-[3-[tert-butyl(dimethyl)silyl]oxypropyl]-2-fluoro-phenyl]-2,2,2-trifluoro-1-hydroxy-ethyl]-1,3,4-oxadiazol-2-yl]-3-(trifluoromethyl)pyridine-2-carboxylate (1.8 g, 2.1521 mmol) in THE (10 mL) at room temperature was added tetrabutylammonium fluoride in THF (20 mL of 1 M, 20 mmol). The mixture was stirred at room temperature for 2 h. Additional tetrabutylammonium fluoride in THF (4 mL of 1 M, 4 mmol) was added and the mixture was stirred for another hour. It was then diluted with ethyl acetate (50 mL) and washed with water (3×50 mL). The organic layer was further washed with brine, dried over anhydrous MgSO₄, filtered and concentrated to afford as a white solid, methyl 5-(tert-butoxycarbonylamino)-6-[5-[2,2,2-trifluoro-1-[2-fluoro-5-(3-hydroxypropyl)phenyl]-1-hydroxy-ethyl]-1,3,4-oxadiazol-2-yl]-3-(trifluoromethyl)pyridine-2-carboxylate (1.4 g, 97%). ESI-MS m/z calc. 638.1611, found 639.6 (M+1)⁺; Retention time: 4.04 minutes (LC Method G).

### Step 8: Preparation of methyl 5-(tert-butoxycarbonylamino)-6-[5-[2,2,2-trifluoro-1-[2-fluoro-5-(3-oxopropyl)phenyl]-1-hydroxy-ethyl]-1,3,4-oxadiazol-2-yl]-3-(trifluoromethyl)pyridine-2-carboxylate

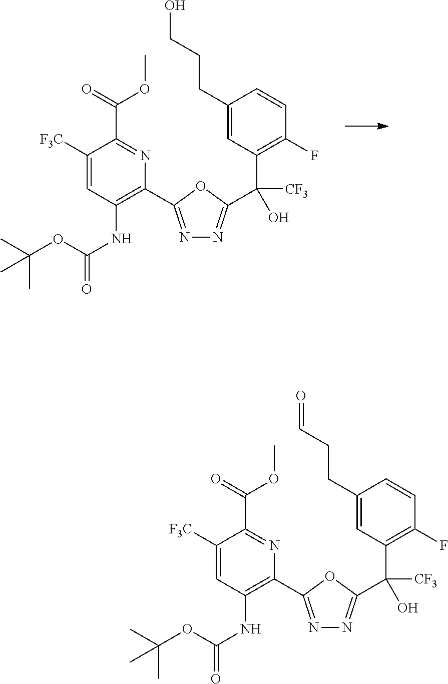 [SMILES: COC(=O)c1nc(-c2nnc(C(O)(c3cc(CCCO)ccc3F)C(F)(F)F)o2)c(NC(=O)OC(C)(C)C)cc1C(F)(F)F ; COC(=O)c1nc(-c2nnc(C(O)(c3cc(CCC=O)ccc3F)C(F)(F)F)o2)c(NC(=O)OC(C)(C)C)cc1C(F)(F)F]

To a solution of methyl 5-(tert-butoxycarbonylamino)-6-[5-[2,2,2-trifluoro-1-[2-fluoro-5-(3-hydroxypropyl)phenyl]-1-hydroxy-ethyl]-1,3,4-oxadiazol-2-yl]-3-(trifluoromethyl)pyridine-2-carboxylate (300 mg, 0.4464 mmol) in DCM (10 mL) at room temperature was added Dess-Martin periodinane (248 mg, 0.5847 mmol) in one portion. The mixture was stirred at room temperature for 3 h. NaS₂O₃ (1 g in 10 ml NaHCO₃) was added. After 10 min, DCM (20 mL) and NaHCO₃ (10 mL) were added, and organic layer was separated. The organic layer was dried over anhydrous MgSO₄, filtered and concentrated. The residue was purified by silica gel chromatography using 0% to 50% ethyl acetate in hexanes to afford as a white solid, methyl 5-(tert-butoxycarbonylamino)-6-[5-[2,2,2-trifluoro-1-[2-fluoro-5-(3-oxopropyl)phenyl]-1-hydroxy-ethyl]-1,3,4-oxadiazol-2-yl]-3-(trifluoromethyl)pyridine-2-carboxylate (288 mg, 96%). ¹H NMR (500 MHz, Chloroform-d) δ 10.52-10.19 (m, 1H), 9.93-9.68 (m, 1H), 9.36 (d, J=4.1 Hz, 1H), 7.71 (d, J=7.3 Hz, 1H), 7.41-7.12 (m, 1H), 7.08-6.77 (m, 1H), 6.15 (s, 1H), 4.01-3.70 (m, 3H), 3.07-2.91 (m, 2H), 2.91-2.73 (m, 2H), 1.71-1.36 (m, 7H) ppm. ESI-MS m/z calc. 636.1455, found 637.4 (M+1)⁺; Retention time: 4.09 minutes (LC Method G).

### Intermediate 23: Preparation of methyl 6-[5-[1-benzyloxy-2,2,2-trifluoro-1-(3-oxopropoxymethyl)ethyl]-1,3,4-oxadiazol-2-yl]-5-(tert-butoxycarbonylamino)-3-(trifluoromethyl)pyridine-2-carboxylate

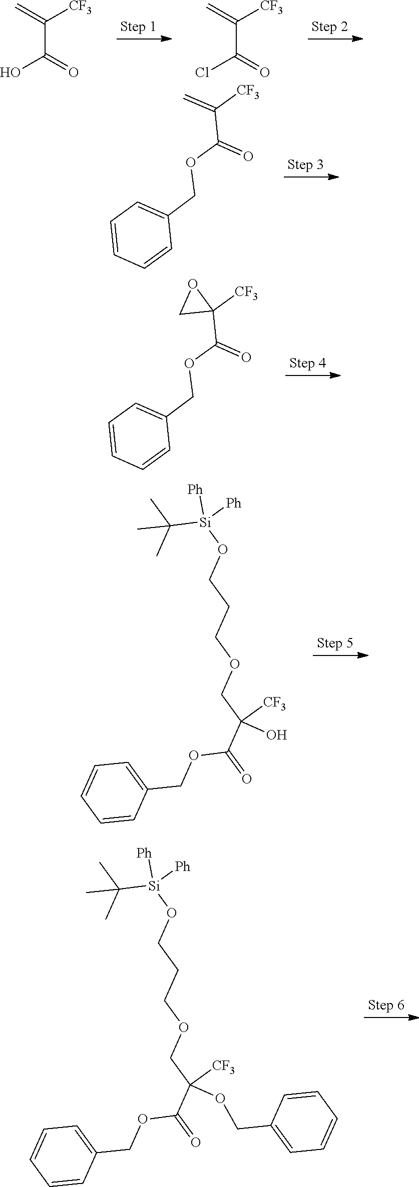

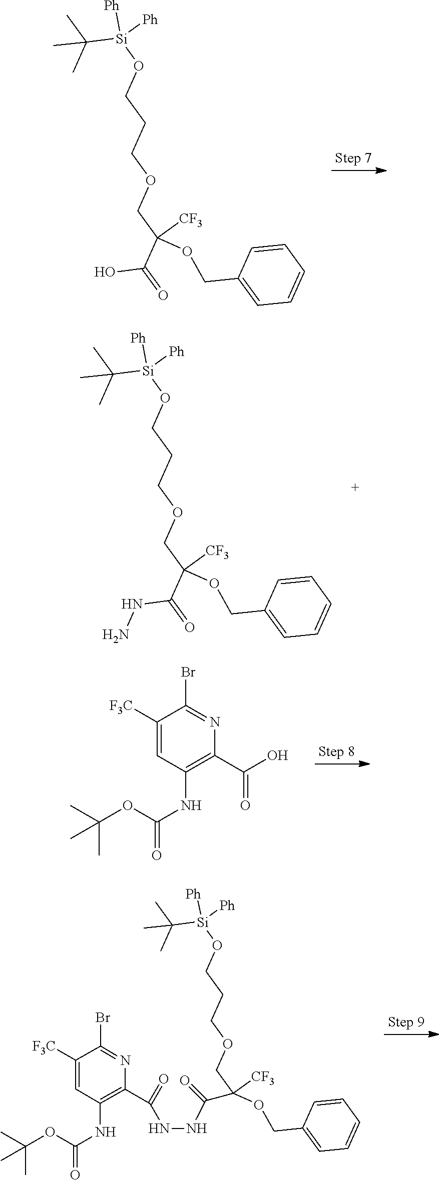

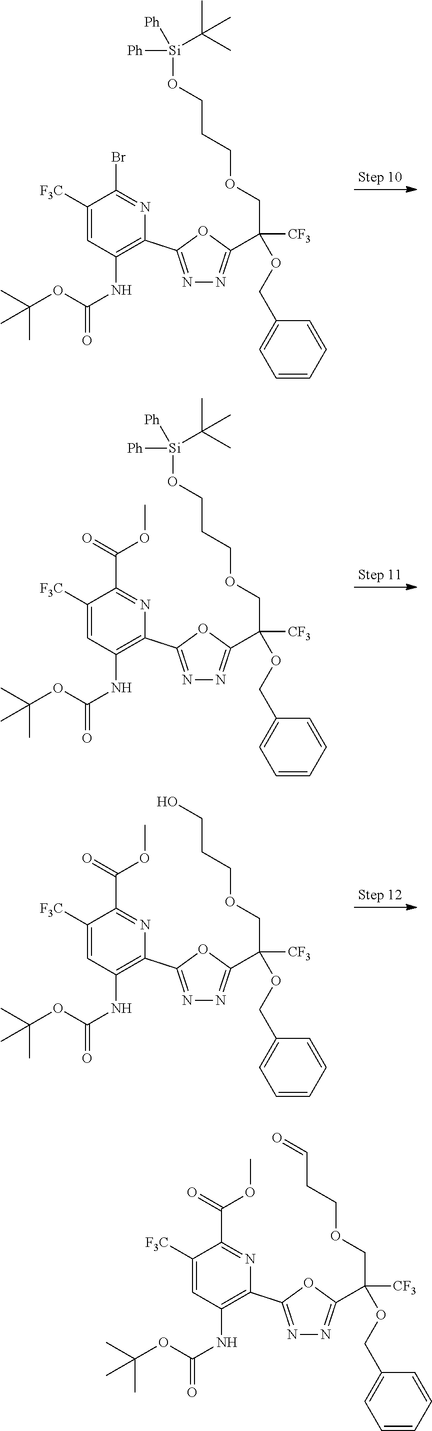

### Step 1: 2-(Trifluoromethyl)prop-2-enoyl chloride

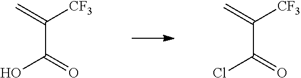

Oxalyl chloride (1.8188 g, 1.25 mL, 14.33 mmol) was added to a stirred solution of 2-(trifluoromethyl)prop-2-enoic acid (2.0 g, 14.279 mmol) and DMF (103.84 mg, 0.11 mL, 1.4206 mmol) in DCM (20 mL) at 0° C. The yellow solution was stirred at room temperature for 1 h. This provided a stock solution of 2-(trifluoromethyl)prop-2-enoyl chloride (20 mL, 0.7 M) for use in further steps.

### Step 2: Benzyl 2-(trifluoromethyl)prop-2-enoate

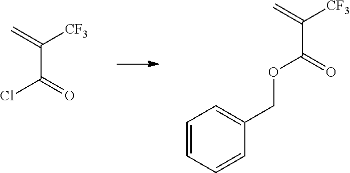

2-(Trifluoromethyl)prop-2-enoyl chloride (3 mL of 0.7 M, 2.1 mmol) followed by triethylamine (217.8 mg, 0.3 mL, 2.1524 mmol) were added to a colorless solution of phenylmethanol (209 mg, 0.2 mL, 1.9327 mmol) in DCM (6 mL) at −40° C. The white suspension was stirred at −40° C. for 1 h and then stirred at 0° C. for 1 h. The colorless solution was quenched with saturated aqueous NH₄Cl (20 mL), extracted with DCM (3×10 mL). The organic layer was dried over sodium sulfate, concentrated under vacuum to give as a white solid, benzyl 2-(trifluoromethyl)prop-2-enoate (429 mg, 96%). ¹H NMR (400 MHz, CDCl₃) δ 7.44-7.33 (m, 5H), 6.76 (q, J=1.6 Hz, 1H), 6.46 (d, J=1.2 Hz, 1H), 5.31 (s, 2H) ppm. ¹⁹F NMR (377 MHz, CDCl₃) δ−65.58 (s, 3F) ppm.

### Step 3: Benzyl 2-(trifluoromethyl)oxirane-2-carboxylate

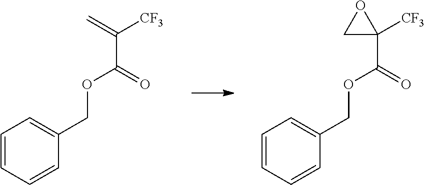 [SMILES: C=C(C(=O)OCc1ccccc1)C(F)(F)F ; O=C(OCc1ccccc1)C1(C(F)(F)F)CO1]

Benzyl 2-(trifluoromethyl)prop-2-enoate (50.45 g, 214.79 mmol) was dissolved in a mixed solvents of dioxane (1000 mL) and water (200 mL). The mixture was cooled in ice water bath. With vigorous stirring, NaHCO₃ (91.2 g, 1.0856 mol) was added, followed by portion-wise addition of potassium peroxymonosulfate (135.5 g) over 80 min. The mixture was stirred at the same temperature for 120 minutes at room temperature. Water (300 mL) and ethyl acetate (300 mL) were added, and layers were separated. The organic layer was washed with brine, dried over anhydrous. MgSO₄, filtered and concentrated. The residue was purified by silica gel chromatography using a gradient from 5% to 50% ethyl acetate in hexanes to afford as a colorless oil, benzyl 2-(trifluoromethyl)oxirane-2-carboxylate (57.49 g, 98%). ¹H NMR (500 MHz, Chloroform-d) δ 7.62-7.28 (m, 5H), 5.33 (d, J=12.3 Hz, 1H), 5.28 (d, J=12.2 Hz, 1H), 3.30-3.18 (m, 2H) ppm.

### Step 4: Benzyl 2-[3-[tert-butyl(diphenyl)silyl]oxypropoxymethyl]-3,3,3-trifluoro-2-hydroxy-propanoate

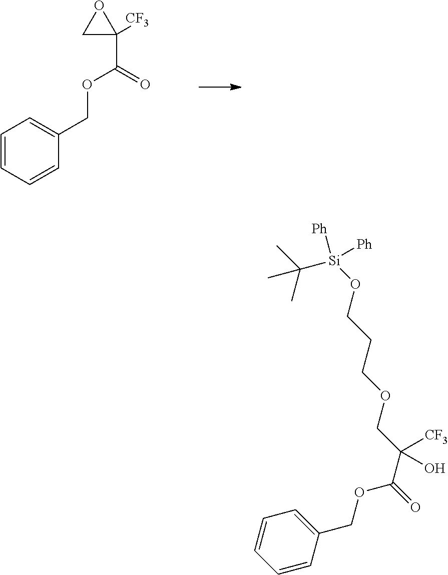 [SMILES: O=C(OCc1ccccc1)C1(C(F)(F)F)CO1 ; CC(C)(C)[Si](OCCCOCC(O)(C(=O)OCc1ccccc1)C(F)(F)F)(c1ccccc1)c1ccccc1]

To a solution of benzyl 2-(trifluoromethyl)oxirane-2-carboxylate (3.9 g, 15.842 mmol) in ethyl acetate (16 mL) was added 3-[tert-butyl(diphenyl)silyl]oxypropan-1-ol (11.6 g, 36.885 mmol) followed by magnesium triflate (5.8 g, 17.988 mmol). The reaction mixture was stirred at 90° C. for 16 hours. The reaction mixture was cooled to room temperature, filtered. The filtrate was concentrated under vacuum and purified by silica gel chromatography using a gradient from 0% to 15% ethyl acetate in hexanes to afford benzyl 2-[3-[tert-butyl(diphenyl)silyl]oxypropoxymethyl]-3,3,3-trifluoro-2-hydroxy-propanoate (5.633 g, 62%). ESI-MS m/z calc. 560.2206, found 561.3 (M+1)⁺; Retention time: 4.48 minutes (LC Method G).

### Step 5: Benzyl 2-benzyloxy-2-[3-[tert-butyl(diphenyl)silyl]oxypropoxymethyl]-3,3,3-trifluoro-propanoate

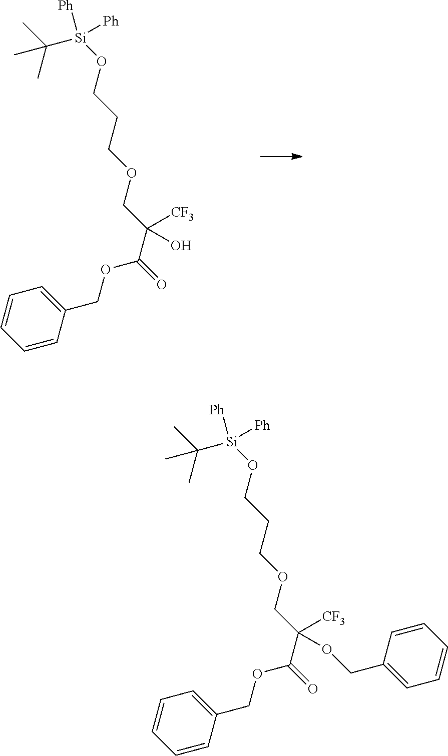 [SMILES: CC(C)(C)[Si](OCCCOCC(O)(C(=O)OCc1ccccc1)C(F)(F)F)(c1ccccc1)c1ccccc1 ; CC(C)(C)[Si](OCCCOCC(OCc1ccccc1)(C(=O)OCc1ccccc1)C(F)(F)F)(c1ccccc1)c1ccccc1]

Into a solution of benzyl 2-[3-[tert-butyl(diphenyl)silyl]oxypropoxymethyl]-3,3,3-trifluoro-2-hydroxy-propanoate (5.633 g, 9.5444 mmol) in anhydrous DMF (55 mL) was added NaH (572.1 mg, 60% w/w, 14.304 mmol) in mineral oil at 0° C. The reaction was stirred at the same temperature for 20 minutes, then bromoethylbenzene (2.2987 g, 1.55 mL, 13.44 mmol) and tetrabutylammonium iodide (352.5 mg, 0.9543 mmol) were added to the reaction mixture. The reaction was slowly raised to room temperature and stirred overnight. The reaction was quenched with saturated ammonium chloride (100 mL) and extracted with ethyl acetate (3×100 mL). The combined organic layers were washed with brine (2×100 mL), dried over anhydrous magnesium sulfate, and concentrated under vacuum. The residue was purified by silica gel chromatography using a gradient from 0% to 20% diethyl ether in hexane to furnish as a clear liquid, benzyl 2-benzyloxy-2-[3-[tert-butyl(diphenyl)silyl]oxypropoxymethyl]-3,3,3-trifluoro-propanoate (4.8 g, 77%). ESI-MS m/z calc. 650.2675, found 651.6 (M+1)⁺; Retention time: 4.75 minutes (LC Method G).

### Step 6: 2-Benzyloxy-2-[3-[tert-butyl(diphenyl)silyl]oxypropoxymethyl]-3,3,3-trifluoro-propanoic Acid

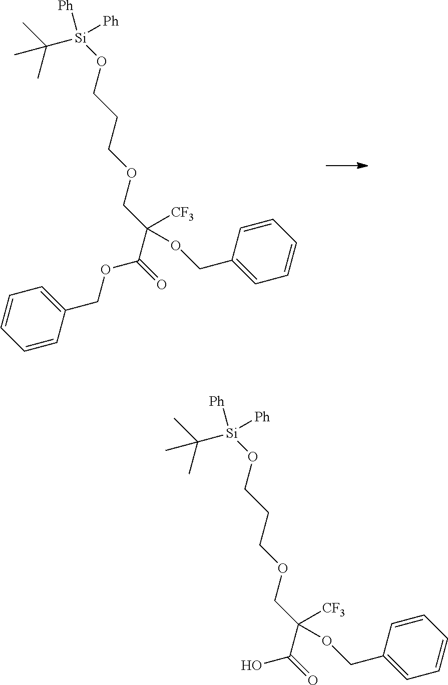 [SMILES: CC(C)(C)[Si](OCCCOCC(OCc1ccccc1)(C(=O)OCc1ccccc1)C(F)(F)F)(c1ccccc1)c1ccccc1 ; CC(C)(C)[Si](OCCCOCC(OCc1ccccc1)(C(=O)O)C(F)(F)F)(c1ccccc1)c1ccccc1]

To a solution of benzyl 2-benzyloxy-2-[3-[tert-butyl(diphenyl)silyl]oxypropoxymethyl]-3,3,3-trifluoro-propanoate (4.62 g, 6.744 mmol) in 1,4-dioxane (65 mL) was added NaOH (20.3 mL of 2 M, 40.6 mmol), and stirred at ambient temperature for overnight. The reaction mixture was acidified with aqueous HCl (1N) to ˜pH 3. The aqueous solution was extracted with ethyl acetate (3×150 mL). The combined ethyl acetate layers were washed with water (2×150 mL), brine (200 mL), dried over anhydrous sodium sulfate, and concentrated under vacuum. The residue was purified by silica gel chromatography using a gradient from 0% to 15% methanol in DCM to furnish as light yellow viscous oil, 2-benzyloxy-2-[3-[tert-butyl(diphenyl)silyl]oxypropoxymethyl]-3,3,3-trifluoro-propanoic acid (3.63 g, 96%). ESI-MS m/z calc. 560.2206, found 561.2 (M+1)⁺; Retention time: 4.39 minutes (LC Method G).

### Step 7: 2-Benzyloxy-2-[3-[tert-butyl(diphenyl)silyl]oxypropoxymethyl]-3,3,3-trifluoro-propanehydrazide

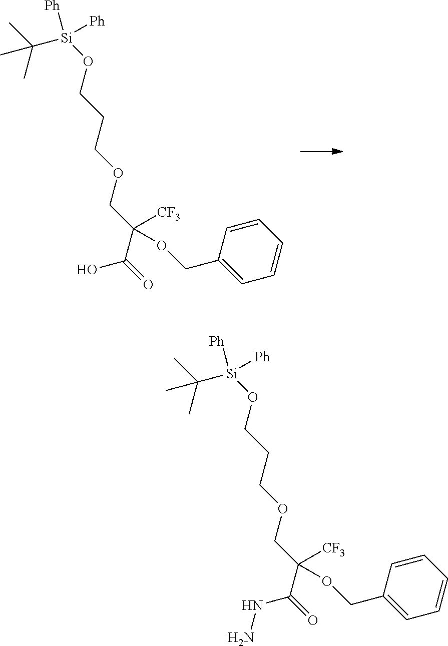 [SMILES: CC(C)(C)[Si](OCCCOCC(OCc1ccccc1)(C(=O)O)C(F)(F)F)(c1ccccc1)c1ccccc1 ; CC(C)(C)[Si](OCCCOCC(OCc1ccccc1)(C(=O)NN)C(F)(F)F)(c1ccccc1)c1ccccc1]

Into a solution of 2-benzyloxy-2-[3-[tert-butyl(diphenyl)silyl]oxypropoxymethyl]-3,3,3-trifluoro-propanoic acid (3.6 g, 5.1366 mmol) in DMF (50 mL) was added HATU (2.92 g, 7.6796 mmol) and DIEA (2.0034 g, 2.7 mL, 15.501 mmol) at ambient temperature. The reaction was stirred at room temperature for 10 minutes. hydrazine (3.2672 g, 3.2 mL, 101.96 mmol) was added to the reaction mixture. The reaction was stirred for 4 hours. Water (100 mL) and ethyl acetate (100 mL) were added to the reaction mixture. Two layers were separated, and the aqueous layer was extracted with ethyl acetate (2×100 mL). The combined ethyl acetate layers were washed with brine (3×100 mL), dried over anhydrous sodium sulfate, and concentrated under vacuum. The residue was purified by silica gel chromatography using a gradient from 50% to 80% diethyl ether in hexanes to furnish as a light pink oil, 2-benzyloxy-2-[3-[tert-butyl(diphenyl)silyl]oxypropoxymethyl]-3,3,3-trifluoro-propanehydrazide (2.71 g, 87%). ESI-MS m/z calc. 574.2475, found 575.4 (M+1)⁺; Retention time: 4.19 minutes (LC Method G).

### Step 8: tert-Butyl N-[2-[[[2-benzyloxy-2-[3-[tert-butyl(diphenyl)silyl]oxypropoxymethyl]-3,3,3-trifluoro-propanoyl]amino]carbamoyl]-6-bromo-5-(trifluoromethyl)-3-pyridyl]carbamate

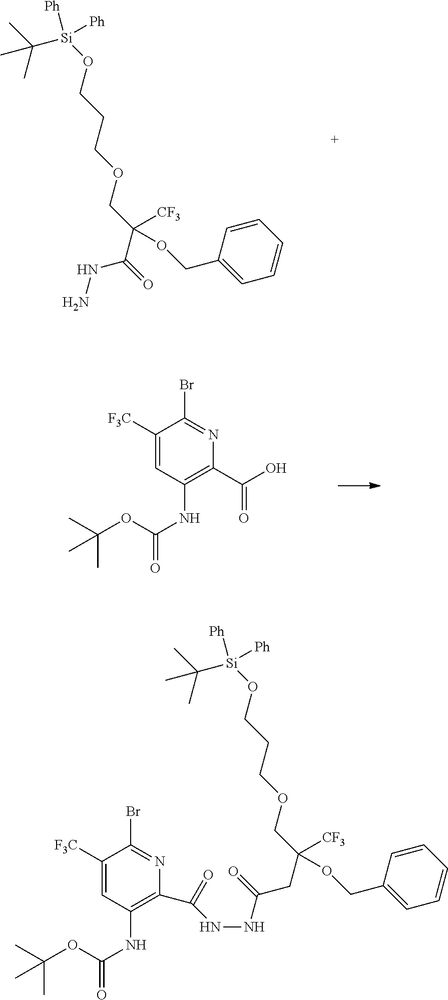 [SMILES: CC(C)(C)[Si](OCCCOCC(OCc1ccccc1)(C(=O)NN)C(F)(F)F)(c1ccccc1)c1ccccc1 ; CC(C)(C)OC(=O)Nc1cc(C(F)(F)F)c(Br)nc1C(=O)O ; CC(C)(C)OC(=O)Nc1cc(C(F)(F)F)c(Br)nc1C(=O)NNC(=O)CC(COCCCO[Si](c1ccccc1)(c1ccccc1)C(C)(C)C)(OCc1ccccc1)C(F)(F)F]

Into a flask was charged with 2-benzyloxy-2-[3-[tert-butyl(diphenyl)silyl]oxypropoxymethyl]-3,3,3-trifluoro-propanehydrazide (2.57 g, 4.4719 mmol) and 6-bromo-3-(tert-butoxycarbonylamino)-5-(trifluoromethyl)pyridine-2-carboxylic acid (1.87 g, 4.8555 mmol) in ethyl acetate (50 mL). Then, pyridine (1.7604 g, 1.8 mL, 22.255 mmol) and T3P in ethyl acetate (3.5758 g, 6.69 mL of 50% w/w, 5.6191 mmol) were added to the reaction mixture. The reaction was stirred at 50° C. for 2 hours. The reaction was diluted with saturated aqueous ammonium chloride (200 mL) and ethyl acetate (150 mL). The aqueous layer was extracted with ethyl acetate (2×150 mL). The combined organic layers were washed with brine (150 mL), dried over anhydrous sodium sulfate, and concentrated under vacuum. The residue was purified by silica gel chromatography using a gradient from 0% to 20% acetone in hexanes to furnish as a pale yellow foamy solid, tert-butyl N-[2-[[[2-benzyloxy-2-[3-[tert-butyl(diphenyl)silyl]oxypropoxymethyl]-3,3,3-trifluoro-propanoyl]amino]carbamoyl]-6-bromo-5-(trifluoromethyl)-3-pyridyl]carbamate (3.49 g, 83%). ESI-MS m/z calc. 940.2302, found 941.6 (M+1)⁺; Retention time: 4.94 minutes (LC Method G).

### Step 9: tert-Butyl N-[2-[5-[l-benzyloxy-1-[3-[tert-butyl(diphenyl)silyl]oxypropoxymethyl]-2,2,2-trifluoro-ethyl]-1,3,4-oxadiazol-2-yl]-6-bromo-5-(trifluoromethyl)-3-pyridyl]carbamate

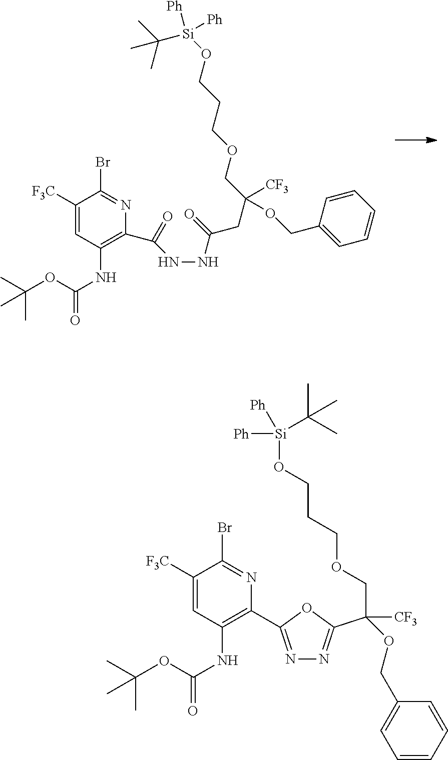 [SMILES: CC(C)(C)OC(=O)Nc1cc(C(F)(F)F)c(Br)nc1C(=O)NNC(=O)CC(COCCCO[Si](c1ccccc1)(c1ccccc1)C(C)(C)C)(OCc1ccccc1)C(F)(F)F ; CC(C)(C)OC(=O)Nc1cc(C(F)(F)F)c(Br)nc1-c1nnc(C(COCCCO[Si](c2ccccc2)(c2ccccc2)C(C)(C)C)(OCc2ccccc2)C(F)(F)F)o1]

Into a solution of tert-butyl N-[2-[[[2-benzyloxy-2-[3-[tert-butyl(diphenyl)silyl]oxypropoxymethyl]-3,3,3-trifluoro-propanoyl]amino]carbamoyl]-6-bromo-5-(trifluoromethyl)-3-pyridyl]carbamate (3.49 g, 3.3350 mmol) and DIEA (1.9663 g, 2.65 mL, 15.214 mmol) in acetonitrile (50 mL) was added TsCl (794.8 mg, 4.1690 mmol). The reaction was stirred at 70° C. for 2 hours. The solvent was removed under vacuum and the residue was directly loaded onto a column and purified by silica gel chromatography using a gradient from 0% to 20% ethyl acetate in hexanes to furnish as a light yellow gel, tert-butyl N-[2-[5-[1-benzyloxy-1-[3-[tert-butyl(diphenyl)silyl]oxypropoxymethyl]-2,2,2-trifluoro-ethyl]-1,3,4-oxadiazol-2-yl]-6-bromo-5-(trifluoromethyl)-3-pyridyl]carbamate (2.98 g, 95%). ESI-MS m/z calc. 922.2196, found 923.7 (M+1)⁺; Retention time: 5.08 minutes (LC Method G).

### Step 10: Methyl 6-[5-[1-benzyloxy-1-[3-[tert-butyl(diphenyl)silyl]oxypropoxymethyl]-2,2,2-trifluoro-ethyl]-1,3,4-oxadiazol-2-yl]-5-(tert-butoxycarbonylamino)-3-(trifluoromethyl)pyridine-2-carboxylate

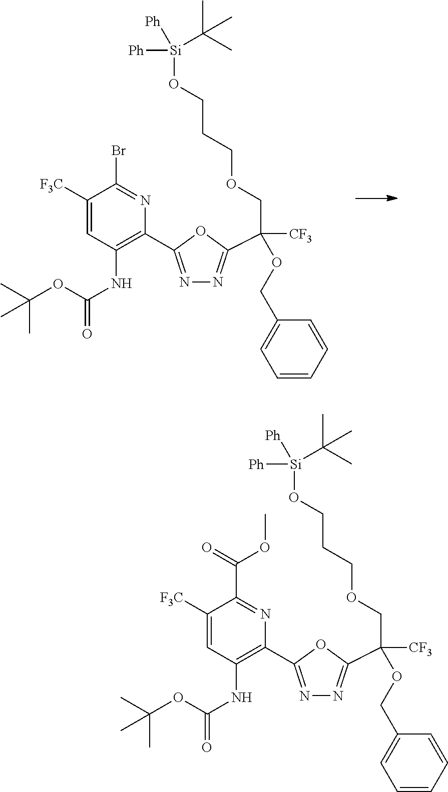

Into a solution of tert-butyl N-[2-[5-[1-benzyloxy-1-[3-[tert-butyl(diphenyl)silyl]oxypropoxymethyl]-2,2,2-trifluoro-ethyl]-1,3,4-oxadiazol-2-yl]-6-bromo-5-(trifluoromethyl)-3-pyridyl]carbamate (2.93 g, 3.1716 mmol) in methanol (35 mL) was added TEA (3.2670 g, 4.5 mL, 32.286 mmol). The solution was purged with nitrogen for 5 minutes. Pd(dppf)Cl₂ (465.2 mg, 0.6358 mmol) was added. The autoclave was sealed and heated at 80° C. for 5 hours under 75 psi of carbon monoxide. The volatile was removed under vacuum. The residue was purified by silica gel chromatography using a gradient from 0% to 20% ethyl acetate in hexanes to furnish as a light yellow gel, methyl 6-[5-[1-benzyloxy-1-[3-[tert-butyl(diphenyl)silyl]oxypropoxymethyl]-2,2,2-trifluoro-ethyl]-1,3,4-oxadiazol-2-yl]-5-(tert-butoxycarbonylamino)-3-(trifluoromethyl)pyridine-2-carboxylate (2.59 g, 90%). ESI-MS m/z calc. 902.3146, found 903.8 (M+1)⁺; Retention time: 4.86 minutes (LC Method G).

### Step 11: Methyl 6-[5-[1-benzyloxy-2,2,2-trifluoro-1-(3-hydroxypropoxymethyl)ethyl]-1,3,4-oxadiazol-2-yl]-5-(tert-butoxycarbonylamino)-3-(trifluoromethyl)pyridine-2-carboxylate

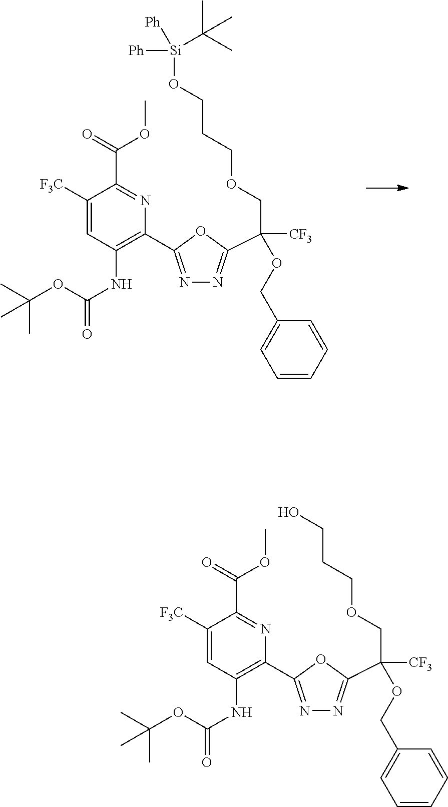 [SMILES: COC(=O)c1nc(-c2nnc(C(COCCCO[Si](c3ccccc3)(c3ccccc3)C(C)(C)C)(OCc3ccccc3)C(F)(F)F)o2)c(NC(=O)OC(C)(C)C)cc1C(F)(F)F ; COC(=O)c1nc(-c2nnc(C(COCCCO)(OCc3ccccc3)C(F)(F)F)o2)c(NC(=O)OC(C)(C)C)cc1C(F)(F)F]

Into a solution of methyl 6-[5-[1-benzyloxy-1-[3-[tert-butyl(diphenyl)silyl]oxypropoxymethyl]-2,2,2-trifluoro-ethyl]-1,3,4-oxadiazol-2-yl]-5-(tert-butoxycarbonylamino)-3-(trifluoromethyl)pyridine-2-carboxylate (2.55 g, 2.8241 mmol) in anhydrous THE (70 mL) was added tetrabutylammonium fluoride in THF (7.1 mL of 1 M, 7.1 mmol) at room temperature. The reaction was stirred at room temperature for overnight. The reaction was diluted with water (150 mL) and ethyl acetate (150 mL). Two layers were separated, and the aqueous layer was extracted with ethyl acetate (2×150 mL). The combined organic layers were washed with brine (250 mL), dried over anhydrous sodium sulfate, and concentrated under vacuum. The residue was purified by silica gel chromatography using a gradient from 0% to 50% ethyl acetate in hexanes to furnish as a white viscous solid, methyl 6-[5-[1-benzyloxy-2,2,2-trifluoro-1-(3-hydroxypropoxymethyl)ethyl]-1,3,4-oxadiazol-2-yl]-5-(tert-butoxycarbonylamino)-3-(trifluoromethyl)pyridine-2-carboxylate (1.783 g, 95%). ESI-MS m/z calc. 664.1968, found 665.1 (M+1)⁺; Retention time: 3.91 minutes (LC Method G).

### Step 12: Methyl 6-[5-[1-benzyloxy-2,2,2-trifluoro-1-(3-oxopropoxymethyl)ethyl]-1,3,4-oxadiazol-2-yl]-5-(tert-butoxycarbonylamino)-3-(trifluoromethyl)pyridine-2-carboxylate

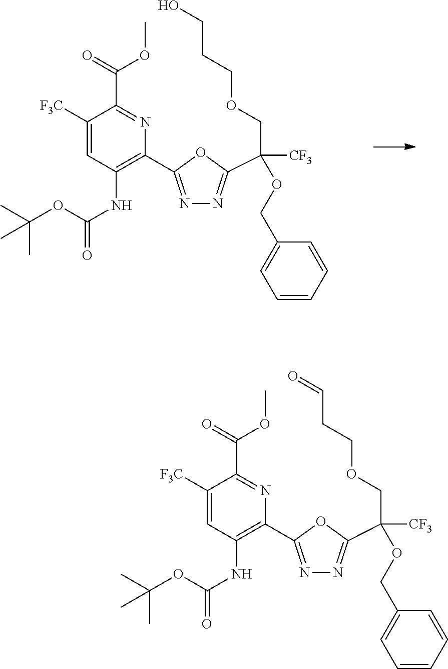 [SMILES: COC(=O)c1nc(-c2nnc(C(COCCCO)(OCc3ccccc3)C(F)(F)F)o2)c(NC(=O)OC(C)(C)C)cc1C(F)(F)F ; COC(=O)c1nc(-c2nnc(C(COCCC=O)(OCc3ccccc3)C(F)(F)F)o2)c(NC(=O)OC(C)(C)C)cc1C(F)(F)F]

Into a solution of methyl 6-[5-[1-benzyloxy-2,2,2-trifluoro-1-(3-hydroxypropoxymethyl)ethyl]-1,3,4-oxadiazol-2-yl]-5-(tert-butoxycarbonylamino)-3-(trifluoromethyl)pyridine-2-carboxylate (1.75 g, 2.6334 mmol) in DCM (65 mL) was added DMP (1.1 g, 2.5935 mmol) at 0° C. The reaction was stirred at 0° C. for 0.5 hour, then raised to room temperature and stirred overnight. The reaction was added saturated Na₂S₂O₃ (170 mL) and saturated NaHCO₃ (130 mL), extracted with DCM (3×150 mL). The combined organic layers were washed with brine (200 mL), dried over sodium sulfate, and concentrated under vacuum. The residue was purified by silica gel chromatography using a gradient from 0% to 50% ethyl acetate in hexanes to furnish as a viscous off-white solid, methyl 6-[5-[1-benzyloxy-2,2,2-trifluoro-1-(3-oxopropoxymethyl)ethyl]-1,3,4-oxadiazol-2-yl]-5-(tert-butoxycarbonylamino)-3-(trifluoromethyl)pyridine-2-carboxylate (1.696 g, 92%). ¹H NMR (500 MHz, DMSO-d₆) δ 10.31 (s, 1H), 9.63 (s, 1H), 9.17 (s, 1H), 7.45 (dd, J=8.0, 1.7 Hz, 2H), 7.41-7.32 (m, 3H), 4.72 (dd, J=17.2, 10.4 Hz, 2H), 4.39 (q, J=11.7, 11.7, 11.7 Hz, 2H), 3.97 (s, 3H), 3.93 (m, 2H), 2.68 (m, 2H), 1.53 (s, 9H) ppm. ESI-MS m/z calc. 662.1811, found 663.4 (M+1)⁺; Retention time: 3.42 minutes (LC Method H).

### Intermediate 24: Preparation of methyl 6-chloro-3-nitro-5-(trifluoromethyl)pyridine-2-carboxylate

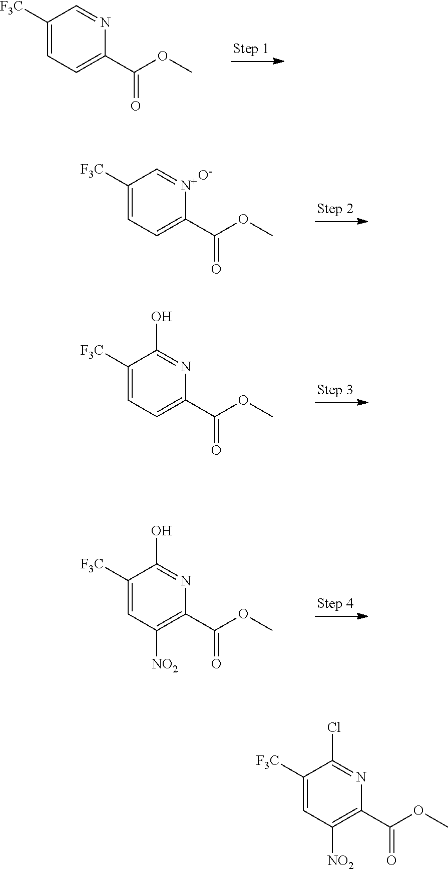

### Step 1: Methyl 1-oxido-5-(trifluoromethyl)pyridin-1-ium-2-carboxylate

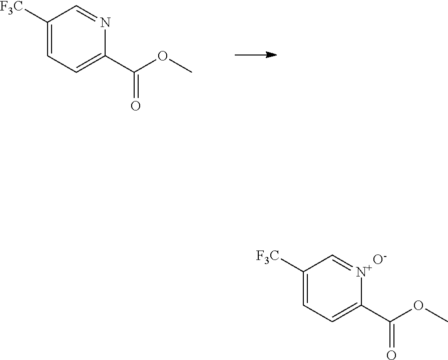

Urea hydrogen peroxide (62.7 g, 646.53 mmol) was added portion-wise to a stirred solution of methyl 5-(trifluoromethyl)pyridine-2-carboxylate (40 g, 191.09 mmol) in 1,2-dichloroethane (300 mL) at 0° C. Trifluoroacetic anhydride (107.70 g, 72 mL, 507.65 mmol) was then added over 30 minutes at a temperature of −10° C., with cooling bath (CO₂/acetone bath). The reaction mixture was then stirred for a further 30 minutes at a temperature of 0° C. and then for 1 hour at ambient temperature. The reaction mixture was then poured into cooled ice-water (600 mL). The mixture was diluted with dichloromethane (300 mL) and then layers were separated. The aqueous phase was extracted with dichloromethane (2×200 mL). The combined organic phase was washed with water (2×300 mL) and brine (1×200 mL), dried over anhydrous sodium sulfate, filtered and concentrated under reduced pressure to give as a light yellow solid, methyl 1-oxido-5-(trifluoromethyl)pyridin-1-ium-2-carboxylate (47.6 g, 90%). ¹H NMR (300 MHz, DMSO-d₆) δ 8.89 (s, 1H), 8.02-7.90 (m, 1H), 7.86-7.72 (m, 1H), 3.89 (s, 3H) ppm. ¹⁹F NMR (282 MHz, DMSO-d₆) δ−62.00 (s, 3F) ppm. ESI-MS m/z calc. 221.02998, found 222.1 (M+1)⁺; Retention time: 1.24 minutes (LC Method E).

### Step 2: Methyl 6-hydroxy-5-(trifluoromethyl)pyridine-2-carboxylate

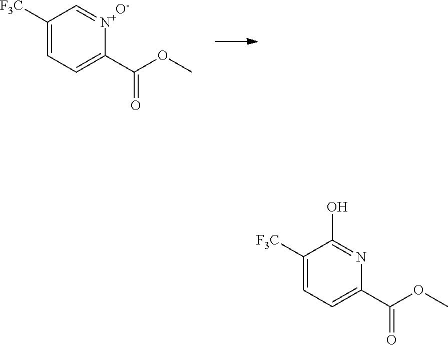

Trifluoroacetic anhydride (291.62 g, 193 mL, 1.3885 mol) was added drop-wise to a mixture of methyl 1-oxido-5-(trifluoromethyl)pyridin-1-ium-2-carboxylate (51.058 g, 230.66 mmol) in DMF (305 mL) at 0° C. The mixture was then stirred at room temperature overnight. The mixture was concentrated under reduced pressure to remove excess of trifluoroacetic acid. The residual DMF solution was poured dropwise to a 0° C. cooled and stirring water volume (1 L). The precipitated solid was collected by filtration and then washed with water (300 mL). The solid was dried under vacuum to afford methyl 6-hydroxy-5-(trifluoromethyl)pyridine-2-carboxylate (45.24 g, 86%) as a white solid. ¹H NMR (300 MHz, CDCl₃) δ 7.90 (d, J 7.2 Hz, 1H), 7.03 (d, J 7.2 Hz, 1H), 4.02 (s, 3H) ppm. One exchangeable proton not observed in NMR. ¹⁹F NMR (282 MHz, CDCl₃) δ−66.39 (s, 3F) ppm. ESI-MS m/z calc. 221.03, found 222.1 (M+1)⁺; Retention time: 1.43 minutes (LC Method E).

### Step 3: Methyl 6-hydroxy-3-nitro-5-(trifluoromethyl)pyridine-2-carboxylate

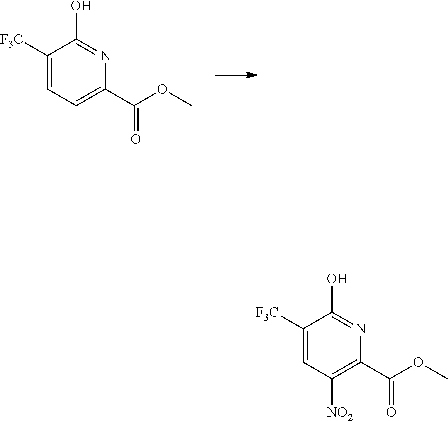 [SMILES: COC(=O)c1ccc(C(F)(F)F)c(O)n1 ; COC(=O)c1nc(O)c(C(F)(F)F)cc1[N+](=O)[O-]]

To an ice-cooled solution of methyl 6-hydroxy-5-(trifluoromethyl)pyridine-2-carboxylate (33.04 g, 149.41 mmol) in sulfuric acid (200 mL of 18.4 M, 3.68 mol) was added nitric acid (13 mL of 15.8 M, 205.4 mmol) dropwise. After 5 min, the ice bath was removed, and the reaction mixture was stirred at 38° C. overnight. The reaction was not completed, nitric acid (3 mL of 15.8 M, 47.4 mmol) was added dropwise at room temperature and the reaction was heated at 38° C. for 4.5 hours. The reaction was poured slowly on ice-cold water (900 mL) and the mixture was cooled at 0° C. for 15 minutes. Then the resultant solid was isolated by filtration and washed with water (600 mL). The solid was dried overnight under vacuum to give as a white solid, methyl 6-hydroxy-3-nitro-5-(trifluoromethyl)pyridine-2-carboxylate (39.49 g, 99%). ¹H NMR (300 MHz, DMSO-d₆) δ 8.54 (s, 1H), 3.95 (s, 3H) ppm. One exchangeable proton not observed in NMR. ¹⁹F NMR (282 MHz, DMSO-d₆) δ−64.56 (s, 3F) ppm. ESI-MS m/z calc. 266.0151, found 267.1 (M+1)⁺; Retention time: 1.64 minutes (LC Method E).

### Step 4: Methyl 6-chloro-3-nitro-5-(trifluoromethyl)pyridine-2-carboxylate

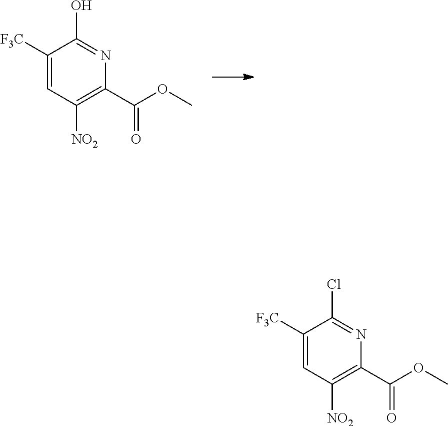 [SMILES: COC(=O)c1nc(O)c(C(F)(F)F)cc1[N+](=O)[O-] ; COC(=O)c1nc(Cl)c(C(F)(F)F)cc1[N+](=O)[O-]]

A mixture of methyl 6-hydroxy-3-nitro-5-(trifluoromethyl)pyridine-2-carboxylate (10 g, 37.575 mmol) and phenyl dichlorophosphate (48.008 g, 34 mL, 227.55 mmol) was heated at 170° C. for 90 minutes. After cooling to room temperature, the mixture was diluted with ethyl acetate (400 mL) and washed with brine (2×200 mL). The organic phase was dried on anhydrous sodium sulfate, filtered and concentrated under reduced pressure. Purification by silica gel chromatography (0% to 15% of ethyl acetate in heptanes) provided methyl 6-chloro-3-nitro-5-(trifluoromethyl)pyridine-2-carboxylate (5.45 g, 50%) as a yellow solid. ¹H NMR (300 MHz, CDCl₃) δ 8.75 (s, 1H), 4.07 (s, 3H) ppm. ¹⁹F NMR (282 MHz, CDCl₃) δ−64.12 (s, 3F) ppm. ESI-MS m/z calc. 283.9812, found 285.0 (M+1)⁺; Retention time: 1.95 minutes (LC Method E).

### Representative Preparation of Primary Amines

### Method 1: [5-[1-(Trifluoromethyl)cyclopropyl]-2-pyridyl]methanamine (Dihydrochloride Salt)

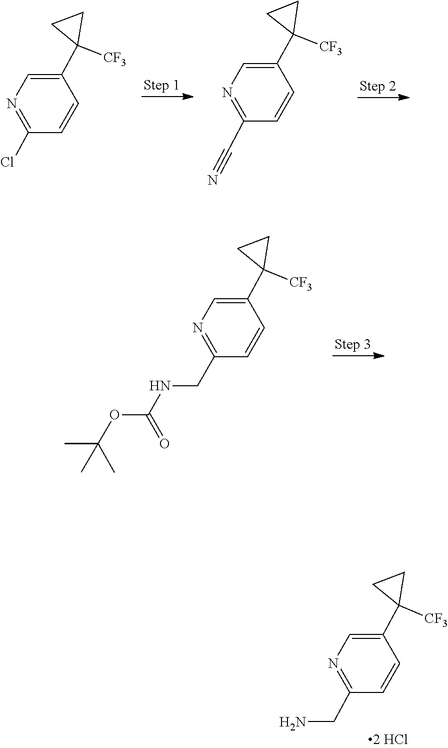 [SMILES: FC(F)(F)C1(c2ccc(Cl)nc2)CC1 ; N#Cc1ccc(C2(C(F)(F)F)CC2)cn1 ; CC(C)(C)OC(=O)NCc1ccc(C2(C(F)(F)F)CC2)cn1 ; NCc1ccc(C2(C(F)(F)F)CC2)cn1]

### Step 1: 5-[1-(Trifluoromethyl)cyclopropyl]pyridine-2-carbonitrile

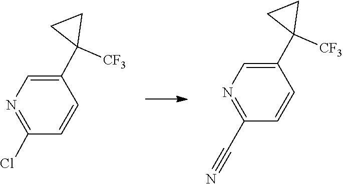 [SMILES: FC(F)(F)C1(c2ccc(Cl)nc2)CC1 ; N#Cc1ccc(C2(C(F)(F)F)CC2)cn1]

Dicyanozinc (65 mg, 0.55 mmol), Pd(dppf)Cl₂ (20 mg, 0.027 mmol), and zinc dust (4 mg, 0.061 mmol) were weighed into a screwcap vial fitted with a stir bar. The vial was then capped and purged with nitrogen. DMF (2.5 mL) and 2-chloro-5-[1-(trifluoromethyl)cyclopropyl]pyridine (200 mg, 0.90 mmol) were added by syringe and the reaction was heated to 150° C. for 90 minutes. The reaction mixture was then cooled to room temperature and partitioned between 30 mL water and 30 mL ethyl acetate. The layers were separated, and the aqueous was extracted with ethyl acetate. The combined organics were washed with brine and dried over sodium sulfate, then concentrated. The resulting crude material was purified by silica gel chromatography using a gradient from 0% to 10% methanol in DCM to give 5-[1-(trifluoromethyl)cyclopropyl]pyridine-2-carbonitrile (118 mg, 62%). ESI-MS m/z calc. 212.05614, found 213.1 (M+1)⁺; Retention time: 0.55 minutes (LC Method S).

### Step 2: tert-Butyl N-[[5-[1-(trifluoromethyl)cyclopropyl]-2-pyridyl]methyl]carbamate

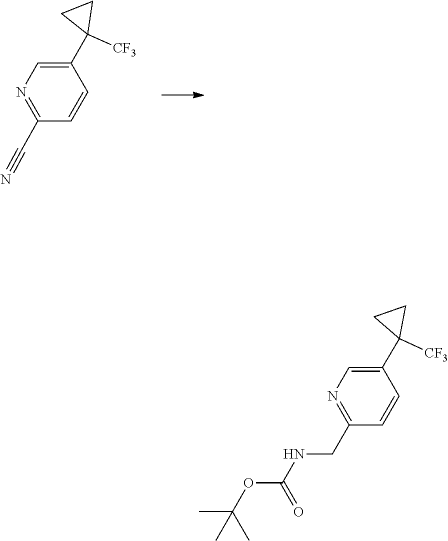 [SMILES: N#Cc1ccc(C2(C(F)(F)F)CC2)cn1 ; CC(C)(C)OC(=O)NCc1ccc(C2(C(F)(F)F)CC2)cn1]

5-[1-(Trifluoromethyl)cyclopropyl]pyridine-2-carbonitrile (118 mg, 0.6 mmol) was dissolved in THF (4 mL) in a nitrogen flushed round bottom flask and cooled to 0° C. in an ice bath. LAH (680 μL of 2 M, 1.36 mmol) in THF was added dropwise to the reaction mixture and stirring was continued for one hour at 0° C. after addition was complete. The reaction mixture was diluted with 5 mL diethyl ether then quenched by the sequential addition of 0.1 mL water, 0.1 mL 15% aqueous NaOH, and 0.3 mL water. The reaction mixture was then warmed to room temperature and stirred for 30 minutes. Sodium sulfate was added and the reaction mixture was filtered. The filtrate was concentrated to give as a light yellow solid, [5-[1-(trifluoromethyl)cyclopropyl]-2-pyridyl]methanamine. ESI-MS m/z calc. 216.08743, found 217.1 (M+1)⁺; Retention time: 0.33 minutes (LC Method T).

The residue was dissolved in methanol (3 mL) and di-tert-butyl dicarbonate (140 mg, 0.6415 mmol) and triethylamine (250 μL, 1.794 mmol) were added. The reaction was stirred at room temperature for 30 minutes then was concentrated by rotary evaporation. The residue was purified by silica gel chromatography using a gradient from 0% to 100% ethyl acetate in hexanes to give tert-butyl N-[[5-[1-(trifluoromethyl)cyclopropyl]-2-pyridyl]methyl]carbamate (65 mg, 23%). ESI-MS m/z calc. 316.13986, found 317.3 (M+1)⁺; Retention time: 0.53 minutes (LC Method T).

### Step 3:[5-[1-(Trifluoromethyl)cyclopropyl]-2-pyridyl]methanamine (Dihydrochloride Salt)

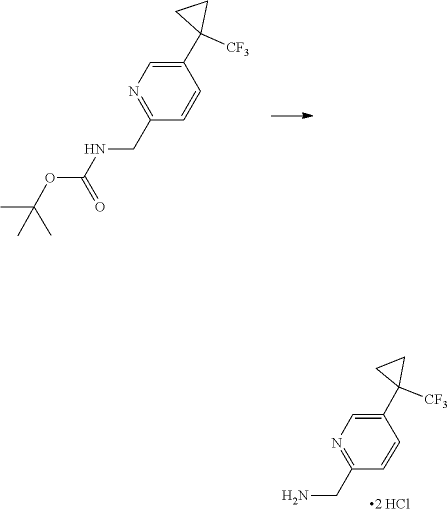

tert-Butyl N-[[5-[1-(trifluoromethyl)cyclopropyl]-2-pyridyl]methyl]carbamate (65 mg, 0.21 mmol) was combined with HCl (1 mL, 4 M, 4 mmol) as a solution in dioxane and DCM (1 mL) and stirred at room temperature for one hour. The reaction mixture was then evaporated to dryness. Dichloromethane and hexanes were added and the reaction was evaporated a second time to give [5-[1-(trifluoromethyl)cyclopropyl]-2-pyridyl]methanamine (dihydrochloride salt) (60 mg, quant.). ESI-MS m/z calc. 216.08743, found 217.1 (M+1)⁺; Retention time: 0.32 minutes (LC Method T).

### Method 2: [3-[[1-(Trifluoromethyl)cyclopropyl]methoxy]phenyl]methanamine (Hydrochloride Salt)

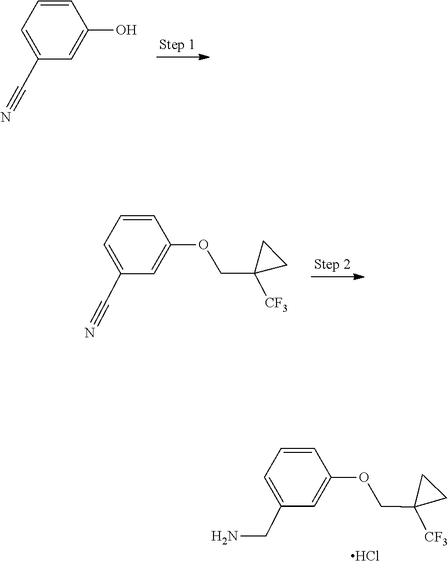 [SMILES: N#Cc1cccc(O)c1 ; N#Cc1cccc(OCC2(C(F)(F)F)CC2)c1 ; NCc1cccc(OCC2(C(F)(F)F)CC2)c1 ; Cl]

### Step 1: 3-[[1-(Trifluoromethyl)cyclopropyl]methoxy]benzonitrile

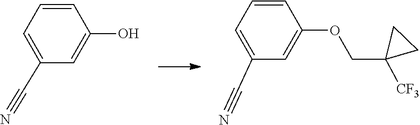 [SMILES: N#Cc1cccc(O)c1 ; N#Cc1cccc(OCC2(C(F)(F)F)CC2)c1]

3-Hydroxybenzonitrile (200 mg, 1.68 mmol) was combined with the [1-(trifluoromethyl)cyclopropyl]methanol (240 mg, 1.713 mmol) and triphenylphosphine (530 mg, 2.02 mmol) in 11 mL THF. The reaction mixture was cooled to 0° C. in an ice bath and diisopropyl azodicarboxylate (450 μL, 470 mg, 2.32 mmol) was added dropwise. After 15 minutes the reaction was warmed to room temperature and stirring was continued at room temperature for 16 hours. The reaction mixture was then warmed to 50° C. for an additional 2 hours. After cooling to room temperature, solvent was removed by rotary evaporation. The resulting residue was dissolved in 50 mL ethyl acetate and washed with 1 M aqueous NaOH. The organics were then washed with brine, dried over sodium sulfate, and concentrated. The crude material was purified by silica gel chromatography using a gradient from 0% to 40% ethyl acetate in hexanes to provide 3-[[1-(trifluoromethyl)cyclopropyl]methoxy]benzonitrile. ESI-MS m/z calc. 241.0714, found 242.0 (M+1)⁺; Retention time: 0.66 minutes (LC Method T).

### Step 2: [3-[[1-(Trifluoromethyl)cyclopropyl]methoxy]phenyl]methanamine (Hydrochloride Salt)

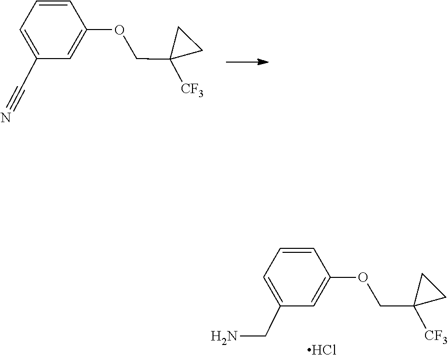 [SMILES: N#Cc1cccc(OCC2(C(F)(F)F)CC2)c1 ; NCc1cccc(OCC2(C(F)(F)F)CC2)c1 ; Cl]

Prepared from 3-[[1-(trifluoromethyl)cyclopropyl]methoxy]benzonitrile as described in method-1, step-2: Isolated [3-[[1-(trifluoromethyl)cyclopropyl]methoxy]phenyl]methanamine (hydrochloride salt). ESI-MS m/z calc. 245.10275, found 246.2 (M+1)⁺; Retention time: 0.4 minutes (LC Method T).

### Method 3: [3-(3,3-Difluorocyclobutyl)phenyl]methanamine (Hydrochloride Salt)

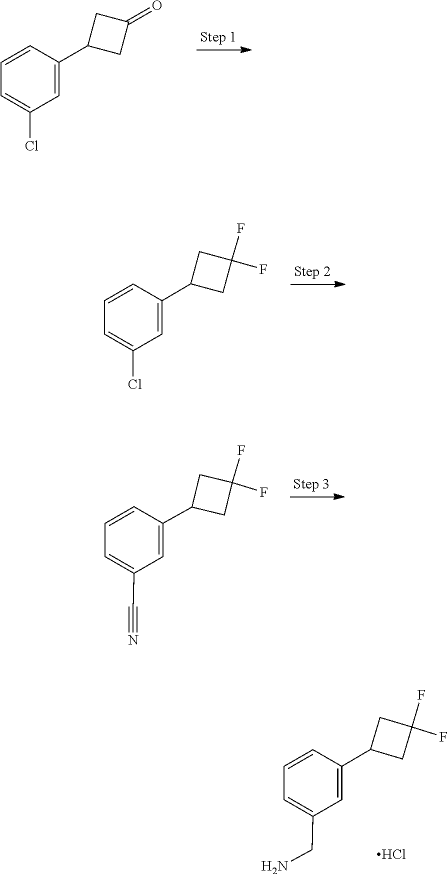 [SMILES: O=C1CC(c2cccc(Cl)c2)C1 ; FC1(F)CC(c2cccc(Cl)c2)C1 ; N#Cc1cccc(C2CC(F)(F)C2)c1 ; NCc1cccc(C2CC(F)(F)C2)c1 ; Cl]

### Step 1: 1-Chloro-3-(3,3-difluorocyclobutyl)benzene

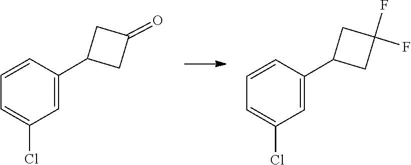 [SMILES: O=C1CC(c2cccc(Cl)c2)C1 ; FC1(F)CC(c2cccc(Cl)c2)C1]

3-(3-Chlorophenyl)cyclobutanone (500 mg, 2.768 mmol) was dissolved in DCM (25 mL) in a nitrogen-purged round bottom flask. The reaction flask was placed in a water bath and diethylaminosulfur trifluoride (1.5 mL, 11.35 mmol) was added dropwise. Stirring was continued at room temperature for 24 hours, then the reaction was placed in an ice water bath and quenched with 100 mL of saturated aqueous sodium bicarbonate (added dropwise at first). The reaction mixture was extracted 3×50 mL with ethyl acetate. The organics were combined, washed with brine, and dried over sodium sulfate then filtered and concentrated. The resulting crude material was purified by silica gel chromatography using 1% to 30% ethyl acetate in hexanes to provide as a colorless oil, 1-chloro-3-(3,3-difluorocyclobutyl)benzene (530 mg, 76%). ¹H NMR (400 MHz, Chloroform-d) δ 7.28-7.24 (m, 1H), 7.24-7.19 (m, 2H), 7.11 (d, J=7.4 Hz, 1H), 3.37 (pd, J=9.2, 2.4 Hz, 1H), 3.01 (tdd, J=13.8, 8.7, 4.6 Hz, 2H), 2.66 (m, 2H) ppm.

### Step 2: 3-(3,3-Difluorocyclobutyl)benzonitrile

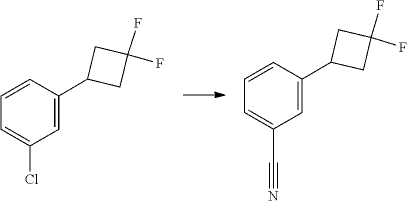 [SMILES: FC1(F)CC(c2cccc(Cl)c2)C1 ; N#Cc1cccc(C2CC(F)(F)C2)c1]

Pd[P(^{t}Bu)₃]₂ (18 mg, 0.03522 mmol) was combined with zinc (8 mg, 0.1223 mmol) and dicyanozinc (42 mg, 0.3576 mmol) then purged with nitrogen. 1-Chloro-3-(3,3-difluorocyclobutyl)benzene (150 mg, 0.5922 mmol) was added as a solution in dimethylacetamide (2 mL) by syringe. The reaction was heated for 4 hours at 90° C. An additional portion of Pd[P(^{t}Bu)₃]₂ (18 mg, 0.03522 mmol) was added, and the reaction vial was re-purged with nitrogen, and the reaction temperature was increased to 115° C. for an additional 4 hours. The reaction was cooled and filtered through Celite and filtrate diluted with ethyl acetate. The filtrate was washed with 15 mL water, and the aqueous layer was extracted with ethyl acetate (2×10 mL). The combined organics were washed with brine, dried over sodium sulfate, and concentrated. The crude material was purified by silica gel chromatography using a gradient from 0% to 40% ethyl acetate in hexanes to give as a colorless oil, 3-(3,3-difluorocyclobutyl)benzonitrile (50 mg, 44%). ESI-MS m/z calc. 193.07031, found 194.1 (M+1)⁺; Retention time: 0.59 minutes (LC Method T).

### Step 3: [3-(3,3-Difluorocyclobutyl)phenyl]methanamine (Hydrochloride Salt)

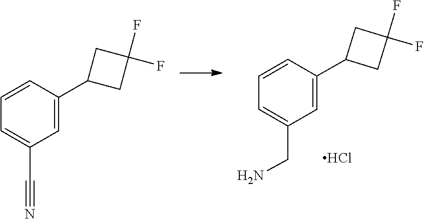 [SMILES: N#Cc1cccc(C2CC(F)(F)C2)c1 ; NCc1cccc(C2CC(F)(F)C2)c1 ; Cl]

3-(3,3-Difluorocyclobutyl)benzonitrile (50 mg, 0.2588 mmol) was stirred in THF (3 mL) in a nitrogen-purged round bottom flask and cooled to 0° C. in an ice bath. LAH (200 μL of 2 M, 0.4 mmol) in THF was added dropwise via syringe. The ice bath was removed and stirring was continued at room temperature for the indicated time. The reaction mixture was then again cooled to 0° C. in an ice bath, and 0.2 mL of water was slowly added dropwise, followed by 0.2 mL of 15% aqueous NaOH, and finally 0.6 mL water. The reaction mixture was then warmed to room temperature and stirred for 3 hours. Stirring was stopped and sodium sulfate was added. The reaction mixture was filtered and concentrated, then the product was suspended in 10 mL dichloromethane and HCl (250 μL of 4 M, 1 mmol) in dioxane was added. The mixture was then concentrated by rotary evaporation, then dichloromethane and hexanes were added and the product was concentrated a second time to give as a white solid, [3-(3,3-difluorocyclobutyl)phenyl]methanamine (hydrochloride salt) (54 mg, 89%). ESI-MS m/z calc. 197.10161, found 198.1 (M+1)⁺; Retention time: 0.34 minutes (LC Method T).

### Method 4: (3-Cyclobutylphenyl)methanamine (Hydrochloride Salt)

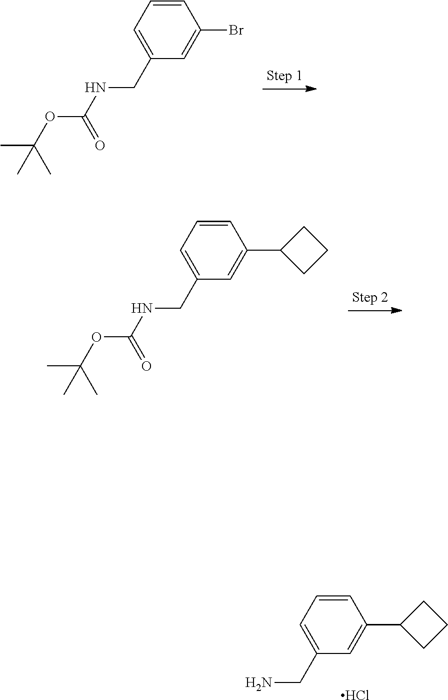 [SMILES: CC(C)(C)OC(=O)NCc1cccc(Br)c1 ; CC(C)(C)OC(=O)NCc1cccc(C2CCC2)c1 ; NCc1cccc(C2CCC2)c1 ; Cl]

### Step 1: tert-Butyl N-[(3-cyclobutylphenyl)methyl]carbamate

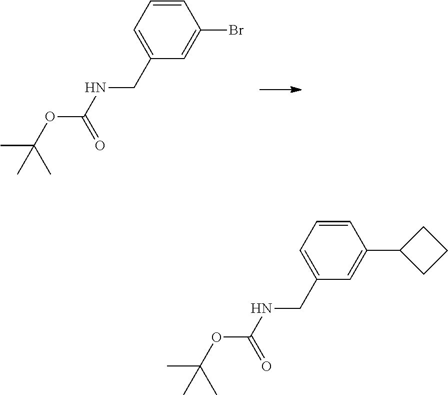 [SMILES: CC(C)(C)OC(=O)NCc1cccc(Br)c1 ; CC(C)(C)OC(=O)NCc1cccc(C2CCC2)c1]

A mixture of tert-butyl N-[(3-bromophenyl)methyl]carbamate (400 mg, 1.4 mmol), bromo(cyclobutyl)zinc (8 mL of 0.5 M, 4 mmol) as a solution in THF, and Pd(PPh₃)₄ (162 mg, 0.14 mmol) in DMF (8 mL) was bubbled with N₂ for 1 min and then heated at 100° C. for 1 h then the mixture was diluted with ether and washed with 1 M HCl, brine, and dried (MgSO₄). A yellow solid was filtered. The filtrate was evaporated. The residue was purified by silica gel chromatography using a gradient from 0% to 20% ethyl acetate in hexanes to provide tert-butyl N-[(3-cyclobutylphenyl)methyl]carbamate (66 mg, 18%). ¹H NMR (400 MHz, Chloroform-d) δ 7.30-7.21 (m, 1H), 7.16-7.05 (m, 3H), 4.79 (s, 1H), 4.30 (d, J=5.9 Hz, 2H), 3.53 (p, J=8.7 Hz, 1H), 2.33 (m, J=10.3, 8.0, 2.4 Hz, 2H), 2.14 (pd, J=9.5, 8.6, 1.8 Hz, 2H), 2.07-1.94 (m, 1H), 1.90-1.77 (m, 1H), 1.47 (s, 9H) ppm. ESI-MS m/z calc. 261.17288, found 523.3 (M+M+1)⁺; Retention time: 0.68 minutes (LC Method T).

### Step 2: (3-Cyclobutylphenyl)methanamine (Hydrochloride Salt)

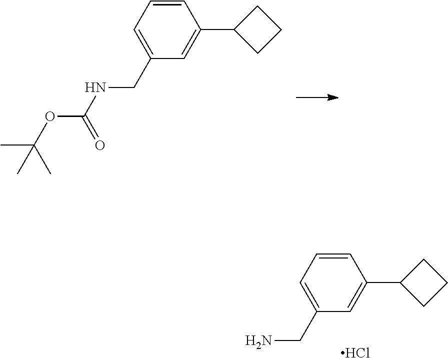 [SMILES: CC(C)(C)OC(=O)NCc1cccc(C2CCC2)c1 ; NCc1cccc(C2CCC2)c1 ; Cl]

A solution of tert-butyl N-[(3-cyclobutylphenyl)methyl]carbamate (66 mg, 0.25 mmol) and HCl (500 μL of 4 M, 2 mmol) as a solution in dioxane was stirred at room temperature for 30 min and then the solvent evaporated. The residue was suspended in ethyl acetate and evaporated to provide as a white solid (3-cyclobutylphenyl)methanamine (hydrochloride salt) (46 mg, 92%). ¹H NMR (400 MHz, DMSO-d₆) δ 8.41 (s, 3H), 7.40 (s, 1H), 7.33 (t, J=7.4 Hz, 1H), 7.29 (d, J=7.4 Hz, 1H), 7.23 (d, J=7.3 Hz, 1H), 3.99 (s, 2H), 3.63-3.46 (m, 1H), 2.30 (qt, J=7.7, 2.4 Hz, 2H), 2.11 (pd, J=9.5, 8.7, 1.8 Hz, 2H), 2.04-1.90 (m, 1H), 1.88-1.72 (m, 1H) ppm. ESI-MS m/z calc. 161.12045, found 162.2 (M+1)⁺; Retention time: 0.33 minutes (LC Method T).

### Method 5: [3-Methoxy-4-(trifluoromethyl)phenyl]methanamine (Hydrochloride Salt)

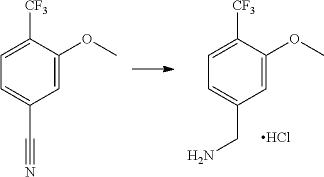

Prepared from 3-methoxy-4-(trifluoromethyl)benzonitrile as described in method 1, steps 2 and 3. Isolated [3-methoxy-4-(trifluoromethyl)phenyl]methanamine (hydrochloride salt). ESI-MS m/z calc. 205.07144, found 206.1 (M+1)⁺; Retention time: 0.34 minutes (LC Method T).

### Method 6: (3-Cyclopropylphenyl)methanamine (Hydrochloride Salt)

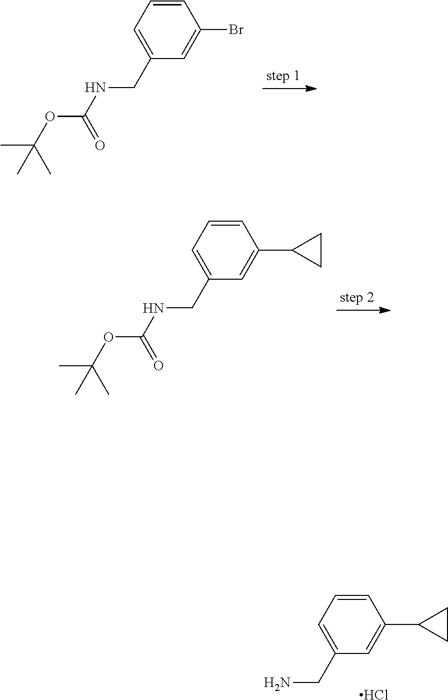 [SMILES: CC(C)(C)OC(=O)NCc1cccc(Br)c1 ; CC(C)(C)OC(=O)NCc1cccc(C2CC2)c1 ; NCc1cccc(C2CC2)c1 ; Cl]

### Step 1: tert-Butyl N-[(3-cyclopropylphenyl)methyl]carbamate

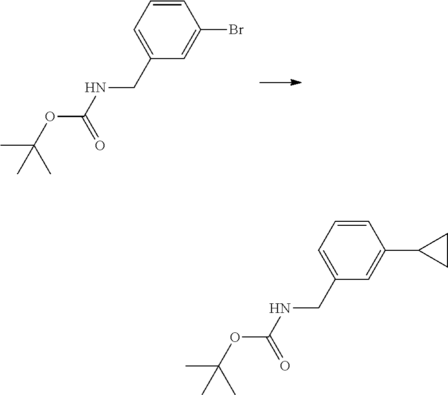 [SMILES: CC(C)(C)OC(=O)NCc1cccc(Br)c1 ; CC(C)(C)OC(=O)NCc1cccc(C2CC2)c1]

A mixture of tert-butyl N-[(3-bromophenyl)methyl]carbamate (305 mg, 1.07 mmol), cyclopropylboronic acid (128 mg, 1.49 mmol), K₃PO₄ (792 mg, 3.73 mmol) and tricyclohexylphosphine (57.5 mg, 0.205 mmol) in toluene (6 mL) and water (305 μL) was degassed then flushed with nitrogen for 10 minutes before adding palladium (II) acetate (24 mg, 0.11 mmol). The mixture was heated at 100° C. for 1 h and then cooled to room temperature. Water (10 mL) was added and the mixture extracted with ethyl acetate (2×15 mL), the combined organic extracts were washed with brine (10 mL), dried over Na₂SO₄ and evaporated. The residue was purified by silica gel chromatography using a gradient from 0% to 15% ethyl acetate in hexanes to afford as a yellow, oil tert-butyl N-[(3-cyclopropylphenyl)methyl]carbamate (200 mg, 76%). ¹H NMR (400 MHz, Chloroform-d) δ 7.19 (t, J=7.6 Hz, 1H), 7.08-6.87 (m, 3H), 4.91 (s, 1H), 4.25 (d, J=5.9 Hz, 2H), 1.92-1.79 (m, 1H), 1.45 (s, 9H), 0.98-0.88 (m, 2H), 0.67 (m, 2H) ppm. ESI-MS m/z calc. 247.15723, Retention time: 0.68 minutes (LC Method T).

### Step 2: (3-Cyclopropylphenyl)methanamine (Hydrochloride Salt)

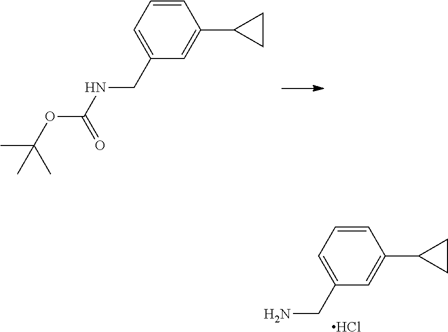 [SMILES: CC(C)(C)OC(=O)NCc1cccc(C2CC2)c1 ; NCc1cccc(C2CC2)c1 ; Cl]

To a solution of tert-butyl N-[(3-cyclopropylphenyl)methyl]carbamate (100 mg, 0.3437 mmol) in DCM (500 μL) was added 4 M HCl in 1,4-dioxane (500 μL of 4 M, 2 mmol) and stirred at room temperature for 25 minutes. The solvent was evaporated to provide as a white solid, (3-cyclopropylphenyl)methanamine (hydrochloride salt) (69 mg, quant.): ESI-MS m/z calc. 147.1048, found 149.17 (M+1)⁺; Retention time: 0.3 minutes (LC Method T).

### General Methods for the Preparation of Secondary Amines

### Method 7

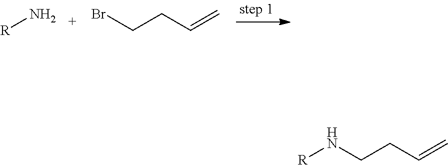

Step 1: A mixture of the primary amine (2 equivalents) and a base such as DIEA, TEA, Cs₂CO₃ (1 equivalent) in DMF (20 to 25 volume equivalents) was stirred at room temperature for 30 min and then 4-bromobut-1-ene or 4-iodobut-1-ene (1 eq) was added and the mixture stirred at room temperature for about 24 h. The mixture was then filtered, and the precipitate washed with ethyl acetate. The filtrate was diluted with ethyl acetate and washed with 1 M NaOH, partitioned and the organic layer extracted with ethyl acetate (3×). The combined organic extracts were washed with brine, dried (MgSO₄), filtered and evaporated. Purification by silica gel chromatography or reverse-phase HPLC provided the desired secondary N-but-3-enylamine as the major product often along with a small amount of undesired tertiary amine as a side product.

### Method 8

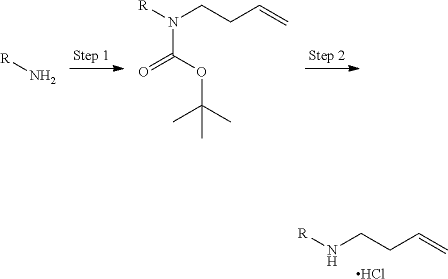

Step 1: A mixture of the primary amine (1 eq) and 4-iodobut-1-ene (1 eq) in THF (20 to 25 volume equivalents) was stirred at 80° C. for about 1 to 2 h to provide the desired secondary amine along with undesired tertiary amine side product. The mixture was cooled to room temperature and TEA (3 equivalents) and di-tert-butyl dicarbonate (1.2 equivalents) were added. The mixture was stirred at room temperature for about 2 h, then diluted with ether, washed with 1 M NaHCO₃, dried (MgSO₄), filtered and evaporated. The residue was purified by silica gel chromatography or reverse phase HPLC to provide the desired N-boc-protected N-but-3-enylamine.

Step 2: A mixture of Boc-protected primary amine (1 eq) and HCl (4 M solution in dioxane, 20 equivalents) was stirred at room temperature for about 16 h, then the solvent evaporated to provide the desired secondary N-but-3-enylamine as hydrochloride salts.

### Method 9

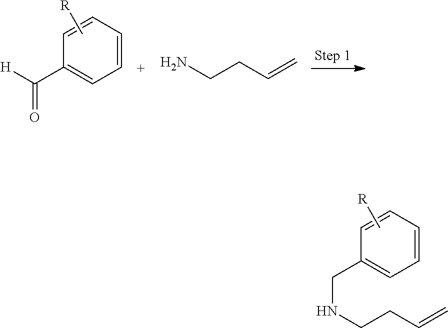

Step 1: To a mixture of the aldehyde (1 eq), but-3-en-1-amine (1.2 equivalents) and acetic acid (0.1 equivalents) in 1,2-dichloroethane (˜30 volume equivalents) was added dry molecular sieves and the mixture was stirred at room temperature for about 1 h. A suitable reducing agent such as sodium triacetoxyborohydride or sodium cyanoborohydride (1.5 equivalents) was added and the resulting mixture was stirred at room temperature overnight. After completion of the reaction, the reaction was quenched with saturated aqueous NaHCO₃, filtered through Celite and extracted with dichloromethane. The organic layer was washed with brine, dried over Na₂SO₄, filtered and concentrated. Purification by silica gel chromatography or reverse phase HPLC provided the desired secondary N-but-3-enylamine.

### Method 10

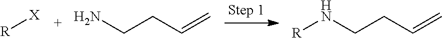

Step 1: A stirred suspension of a base such as potassium carbonate, cesium carbonate, cesium fluoride or cesium acetate (1.2 equivalents) and iodocopper (1 equivalent) in toluene (2 volume equivalents) in a Teflon capped vial was degassed by bubbling nitrogen through the mixture for 5 minutes and then sealed. Under nitrogen atmosphere, the halide (1 equivalent) and but-3-en-1-amine (2 equivalents) were added followed by degassed DMF (5 volume equivalents) and the resulting mixture was sealed and heated at 90° C. overnight. The mixture was cooled to room temperature and diluted with ammonium chloride (ammonium hydroxide added until a blue color persisted) then extracted with ethyl acetate. The organic fraction was washed once with brine, dried over MgSO₄, filtered and concentrated to a brown syrup which was purified by silica gel chromatography or reverse phase HPLC providing the desired secondary N-but-3-enylamine.

### Method 11

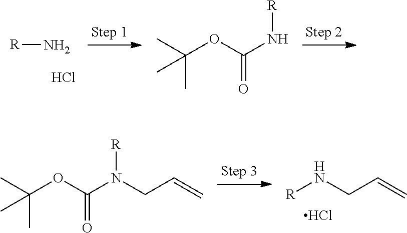

Step 1: To a mixture of amine (hydrochloride salt) (1 equivalent) and NEt₃ (3 equivalents) in THF (20 volume equivalents) was added di-tert-butyl dicarbonate (1.1 equivalents) at room temperature. The mixture was stirred at room temperature for about 1 to 2 h then diluted with ether, washed with 1 M NaHCO₃, dried (MgSO₄), filtered and evaporated. The residue was purified by silica gel chromatography to provide the mono-Boc protected amine.

Step 2: To a (cooled in ice bath) solution of mono-Boc protected amine (1 equivalent) in DMF (10 volume equivalents) was added a base such as [bis(trimethylsilyl)amino]sodium (2 equivalents). After stirring at 0° C. for 5 min, 3-bromo-1-propene (3 equivalents) was added dropwise. The temperature was raised to room temperature and stirred at this temperature for about 1 to 2 h. Then, the mixture was diluted with 1 M NH₄Cl and ether and the aqueous layer was discarded. The organic layer was washed with water then brine, dried (MgSO₄), filtered and evaporated. The residue was purified by silica gel chromatography or reverse phase HPLC to provide the Boc protected allyl amine.

Step 3: A mixture of the Boc protected allyl amine (1 equivalent) and HCl (10 eq, 4 M, in dioxane) was stirred at room temperature for about 2 h, then the solvent was evaporated and the residue was co-evaporated with THE to provide the hydrochloride salt of the substituted N-allyl-amine.

### Method 12

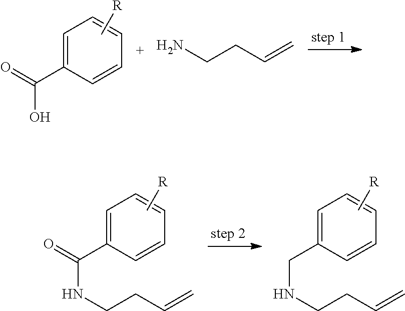

Step 1: To a solution of acid (1 equivalent) and but-3-en-1-amine (1.1 equivalents) in DMF (12 volume equivalents) was added DIEA (2.5 equivalents) and HATU (1.2 equivalents) were added. The resulting mixture was stirred at room temperature for 2 to 3 h. After this time, it was quenched with 1N HCl solution and extracted with ethyl acetate. The combined organic extracts were washed with H₂O and saturated aqueous NaCl solution, then dried over Na₂SO₄, filtered, and evaporated in vacuo. Purification by silica gel chromatography using ethyl acetate and hexanes provided the desired amides.

Step 2: The amide (1 equivalent) was dissolved in diethyl ether (15 to 20 volume equivalents) and cooled to 0° C. A THE solution of LiAlH₄ (1 to 2 equiv.) was added, and the resulting mixture was stirred at room temperature for about 2 h. The mixture was poured slowly into cold water and was extracted with ethyl acetate. The combined organic extracts were washed with H₂O, 1N NaOH solution, H₂O, and saturated aqueous NaCl solution, then dried over Na₂SO₄, filtered, and evaporated in vacuo. Purification by silica gel chromatography or reverse-phase preparative chromatography provided the secondary amines as free base or salt depending upon the purification methods.

### Preparation of Final Compounds

### Example 1: Preparation of 17-amino-6,15-bis(trifluoromethyl)-19-oxa-3,4,18-triazatricyclo[12.3.1.12,5]nonadeca-1(18),2,4,14,16-pentaen-6-ol (Compound 1), 17-amino-6,15-bis(trifluoromethyl)-19-oxa-3,4,18-triazatricyclo[12.3.1.12,5]nonadeca-1(18),2,4,14,16-pentaen-6-ol (enantiomer 1) (Compound 2), and 17-amino-6,15-bis(trifluoromethyl)-19-oxa-3,4,18-triazatricyclo[12.3.1.12,5]nonadeca-1(18),2,4,14,16-pentaen-6-ol (Enantiomer 2) (Compound 3)

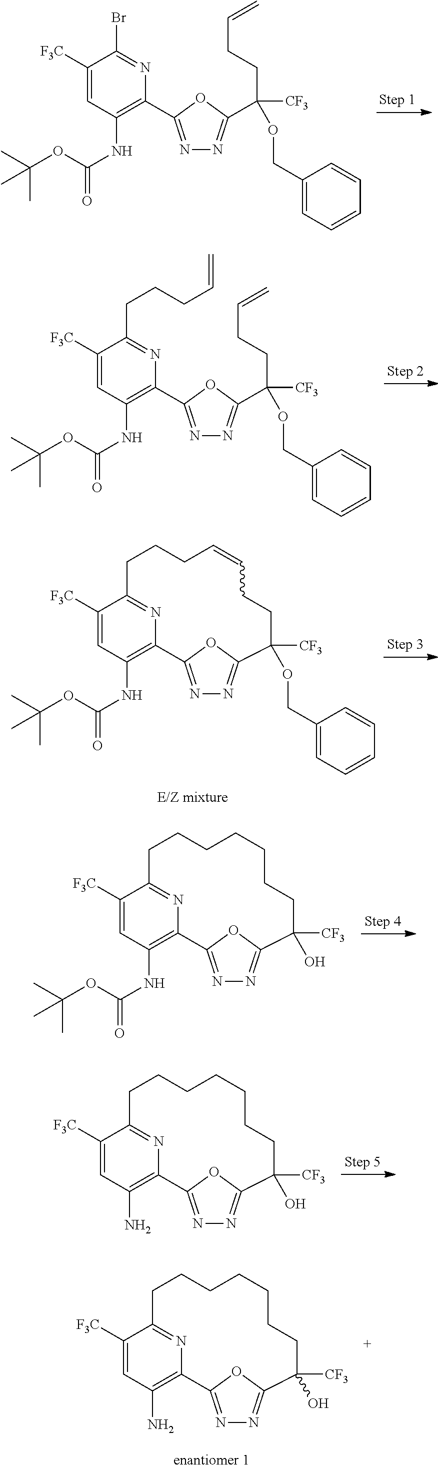 [SMILES: C=CCCC(OCc1ccccc1)(c1nnc(-c2nc(Br)c(C(F)(F)F)cc2NC(=O)OC(C)(C)C)o1)C(F)(F)F ; C=CCCCc1nc(-c2nnc(C(CCC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(OCc1ccccc1)(C(F)(F)F)CCC=CCCC2 ; CC(C)(C)OC(=O)Nc1cc(C(F)(F)F)c2nc1C1=NN=C(O1)C(O)(C(F)(F)F)CCCCCCC2 ; Nc1cc(C(F)(F)F)c2nc1C1=NN=C(O1)C(O)(C(F)(F)F)CCCCCCC2 ; Nc1cc(C(F)(F)F)c2nc1C1=NN=C(O1)C(O)(C(F)(F)F)CCCCCCC2]

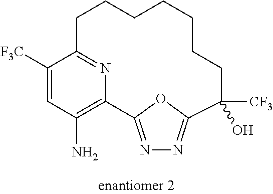 [SMILES: Nc1cc(C(F)(F)F)c2nc1C1=NN=C(O1)C(O)(C(F)(F)F)CCCCCCC2]

### Step 1: tert-Butyl N-[2-[5-[1-benzyloxy-1-(trifluoromethyl)pent-4-enyl]-1,3,4-oxadiazol-2-yl]-6-pent-4-enyl-5-(trifluoromethyl)-3-pyridyl]carbamate

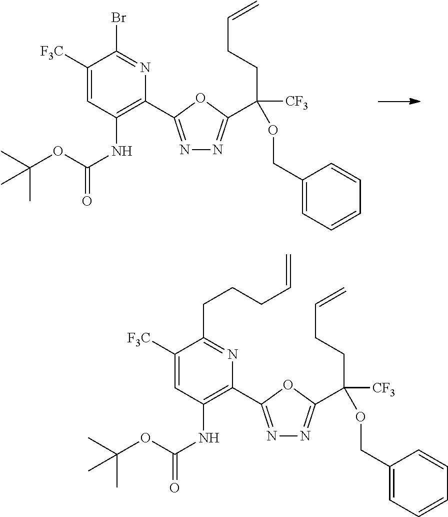

An oven-dried screw-cap test tube, which was equipped with a magnetic stir bar and fitted with a Teflon septum, was charged with tert-butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate (300 mg, 0.534 mmol) and dry THF (2 mL). Bromo(pent-4-enyl)zinc (2.7 mL of 0.5 M, 1.35 mmol) was added and the vessel was evacuated and backfilled with argon (this process was repeated a total of 3 times). The solution was cooled to 0° C. in an ice bath and Pd(OAc)₂ (6 mg, 0.027 mmol) and C-Phos (23.4 mg, 0.054 mmol) were quickly added. The ice-bath was removed, and the reaction was stirred at 25° C. overnight. The reaction mixture was quenched by adding saturated aqueous ammonium chloride solution and extracted with ethyl acetate (50 mL). The organic layer was washed with brine, dried over anhydrous sodium sulfate and concentrated under reduced pressure. The residue was purified by silica gel chromatography using a gradient from 0% to 20% ethyl acetate in heptanes which gave as a colorless oil, tert-butyl N-[2-[5-[1-benzyloxy-1-(trifluoromethyl)pent-4-enyl]-1,3,4-oxadiazol-2-yl]-6-pent-4-enyl-5-(trifluoromethyl)-3-pyridyl]carbamate (120 mg, 35%). ¹H NMR (300 MHz, CDCl₃) δ 1.56 (s, 9H), 1.86-1.96 (m, 2H), 2.14-2.29 (m, 3H), 2.30-2.54 (m, 3H), 2.97 (t, J=7.6 Hz, 2H), 4.69 (d, J=10.6, 1H), 4.83 (d, J=10.6, 1H), 4.96-5.08 (m, 4H), 5.70-5.89 (m, 2H), 7.30-7.39 (m, 3H), 7.43-7.47 (m, 2H), 9.1 (s, 1H), 10.08 (s, 1H) ppm. ¹⁹F NMR (282 MHz, CDCl₃) δ−72.83 (s, 3F), −61.41 (s, 3F) ppm. ESI-MS m/z calc. 640.2484, Retention time: 3.13 minutes (LC Method K).

### Step 2: tert-Butyl N-[6-benzyloxy-6,15-bis(trifluoromethyl)-19-oxa-3,4,18-triazatricyclo[12.3.1.12,5]nonadeca-1(17),2,4,9,14(18),15-hexaen-17-yl]carbamate (E/Z Mixture)

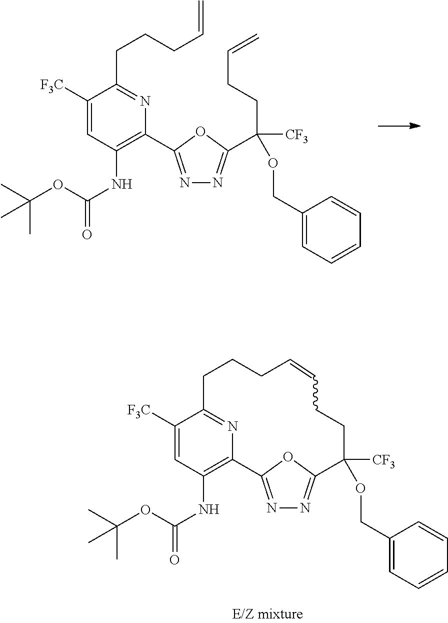 [SMILES: C=CCCCc1nc(-c2nnc(C(CCC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(OCc1ccccc1)(C(F)(F)F)CCC=CCCC2]

tert-Butyl N-[2-[5-[1-benzyloxy-1-(trifluoromethyl)pent-4-enyl]-1,3,4-oxadiazol-2-yl]-6-pent-4-enyl-5-(trifluoromethyl)-3-pyridyl]carbamate (455 mg, 0.7103 mmol) was dissolved in toluene (270 mL). The flask was evacuated and backfilled with nitrogen (3 cycles), and then further degassed by purging with nitrogen for 30 minutes. Benzylidene[1,3-bis(2,4,6-trimethylphenyl)-2-imidazolidinylidene]dichloro(tricyclohexylphosphine)ruthenium (133 mg, 0.1563 mmol) was added at room temperature and the mixture was stirred at 100° C. overnight. The black reaction mixture was removed from hot oil bath and allowed to cool to room temperature. Di(ethylene glycol) vinyl ether (387.20 mg, 0.4 mL, 2.9298 mmol) was added at 0° C. and the mixture was stirred at room temperature for 10 minutes and concentrated under reduced pressure. The residue was purified by silica gel chromatography using a gradient from 0% to 10% ethyl acetate in heptanes which gave as a colorless oil, tert-butyl N-[6-benzyloxy-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (243 mg, 39%). ¹H NMR (300 MHz, CDCl₃) δ 1.55 (s, 9H), 1.80-2.20 (m, 2H), 2.20-2.40 (m, 2H), 2.40-2.70 (m, 4H), 2.90-3.20 (m, 2H), 4.88 (s, 2H), 5.40-5.60 (m, 2H), 7.20-7.40 (m, 5H), 9.13 (s, 1H), 9.51 (s, 1H) ppm. ¹⁹F NMR (282 MHz, CDCl₃) δ−74.62 (s, 3F), −62.72 (s, 3F) ppm.

### Step 3: tert-Butyl N-[6-hydroxy-6,15-bis(trifluoromethyl)-19-oxa-3,4,18-triazatricyclo[12.3.1.12,5]nonadeca-1(17),2,4,14(18),15-pentaen-17-yl]carbamate

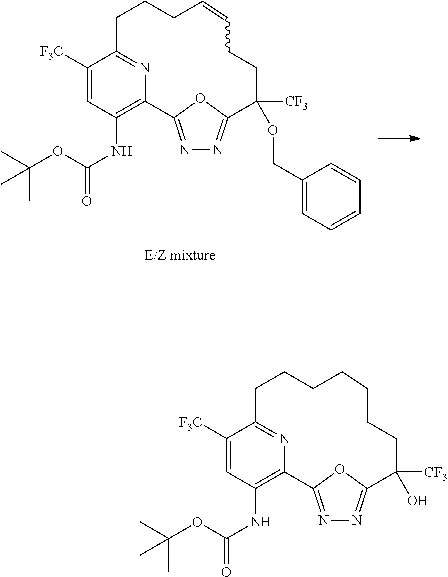 [SMILES: CC(C)(C)OC(=O)Nc1cc(C(F)(F)F)c2nc1C1=NN=C(O1)C(OCc1ccccc1)(C(F)(F)F)CCC=CCCC2 ; CC(C)(C)OC(=O)Nc1cc(C(F)(F)F)c2nc1C1=NN=C(O1)C(O)(C(F)(F)F)CCCCCCC2]

A mixture of tert-butyl N-[6-benzyloxy-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (470 mg, 0.5371 mmol), Silicat Pd (959 mg, 0.24 mmol/g, 0.2302 mmol) and methanol (15 mL) was hydrogenated overnight at room temperature with a hydrogen balloon. The mixture was diluted with ethyl acetate (30 mL), filtered through a pad of Celite. The filtrate was concentrated under reduced pressure. The residue was purified by silica gel chromatography using a gradient from 0% to 10% ethyl acetate in heptanes which gave as a white solid, tert-butyl N-[6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (280 mg, 98%). ¹H NMR (300 MHz, DMSO-d₆) ppm 1.25-1.65 (s, 16H), 1.65-1.90 (m, 3H), 2.05-2.25 (m, 2H), 2.83-3.11 (m, 2H), 7.70 (s, 1H), 8.80 (s, 1H) ppm. ¹⁹F NMR (282 MHz, DMSO-d₆) ppm −78.2 (s, 3F), −61.4 (s, 3F) ppm. ESI-MS m/z calc. 524.18585, found 525.2 (M+1)⁺; Retention time: 4.4 minutes (LC Method C).

### Step 4: 17-Amino-6,15-bis(trifluoromethyl)-19-oxa-3,4,18-triazatricyclo[12.3.1.12,5]nonadeca-1(18),2,4,14,16-pentaen-6-ol (Compound 1)

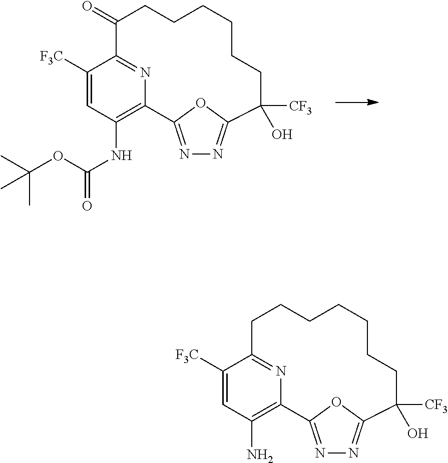 [SMILES: CC(C)(C)OC(=O)Nc1cc(C(F)(F)F)c2nc1C1=NN=C(O1)C(O)(C(F)(F)F)CCCCCCC2=O ; Nc1cc(C(F)(F)F)c2nc1C1=NN=C(O1)C(O)(C(F)(F)F)CCCCCCC2]

TFA (4.44 g, 3 mL, 38.94 mmol) was added to a solution of tert-butyl N-[6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (270 mg, 0.5148 mmol) in dichloromethane (9 mL) at room temperature. The mixture was stirred at room temperature for 30 minutes. The mixture was concentrated under reduced pressure and the residue was dissolved in dichloromethane (50 mL), washed with 5% aqueous NaHCO₃ solution. The organic layer was separated, dried over anhydrous sodium sulfate and concentrated under reduced pressure. The residue was purified by silica gel chromatography using a gradient from 0% to 20% ethyl acetate in heptanes which gave as a white solid, 17-amino-6,15-bis(trifluoromethyl)-19-oxa-3,4,18-triazatricyclo[12.3.1.12,5]nonadeca-1(18),2,4,14,16-pentaen-6-ol (150 mg, 68%). ¹H NMR (300 MHz, DMSO-d₆) ppm 1.25-1.65 (m, 7H), 1.70-1.85 (m, 3H), 2.10-2.20 (m, 2H), 2.67-2.98 (m, 2H), 6.73 (s, 2H), 7.61 (s, 1H), 7.68 (s, 1H) ppm. ¹⁹F NMR (282 MHz, DMSO-d₆) ppm −78.1 (s, 3F), −61.5 (s, 3F) ppm. ESI-MS m/z calc. 424.1334, found 425.1 (M+1)⁺; Retention time: 3.72 minutes (LC Method C).

### Step 5: 17-Amino-6,15-bis(trifluoromethyl)-19-oxa-3,4,18-triazatricyclo[12.3.1.12,5]nonadeca-1(18),2,4,14,16-pentaen-6-ol (enantiomer 1) (Compound 2) and 17-amino-6,15-bis(trifluoromethyl)-19-oxa-3,4,18-triazatricyclo[12.3.1.12,5]nonadeca-1(18),2,4,14,16-pentaen-6-ol (Enantiomer 2) (Compound 3)

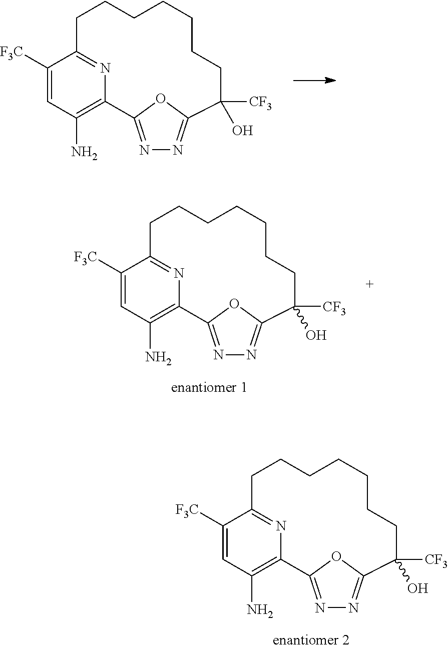 [SMILES: Nc1cc(C(F)(F)F)c2nc1-c1nnc(o1)C(O)(C(F)(F)F)CCCCCCC2 ; Nc1cc(C(F)(F)F)c2nc1-c1nnc(o1)C(O)(C(F)(F)F)CCCCCCC2 ; Nc1cc(C(F)(F)F)c2nc1-c1nnc(o1)C(O)(C(F)(F)F)CCCCCCC2]

Racemic 17-amino-6,15-bis(trifluoromethyl)-19-oxa-3,4,18-triazatricyclo[12.3.1.12,5]nonadeca-1(18),2,4,14,16-pentaen-6-ol (142 mg, 0.3336 mmol) was purified by chiral SFC using a Phenomenex LUX-4 column (250×10 mm, 5 m particle size), 14% MeOH (no modifier) and 86% CO₂ as a gradient using a flow rate 70 mL/min with an injection volume of ˜23 mg/ml in methanol (no modifier) giving as the first eluting enantiomer 17-amino-6,15-bis(trifluoromethyl)-19-oxa-3,4,18-triazatricyclo[12.3.1.12,5]nonadeca-1(18),2,4,14,16-pentaen-6-ol (enantiomer 1) (35 mg, 48%). ESI-MS m/z calc. 424.1334, found 425.1 (M+1)⁺; Retention time: 2.95 minutes. The later eluting enantiomer gave 17-amino-6,15-bis(trifluoromethyl)-19-oxa-3,4,18-triazatricyclo[12.3.1.12,5]nonadeca-1(18),2,4,14,16-pentaen-6-ol (enantiomer 2) (47 mg, 65%). ESI-MS m/z calc. 424.1334, found 425.1 (M+1)⁺; Retention time: 2.95 minutes (LC Method FF).

### Example 2: Preparation of 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (enantiomer 1) (Compound 4) and 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (Enantiomer 2) (Compound 5)

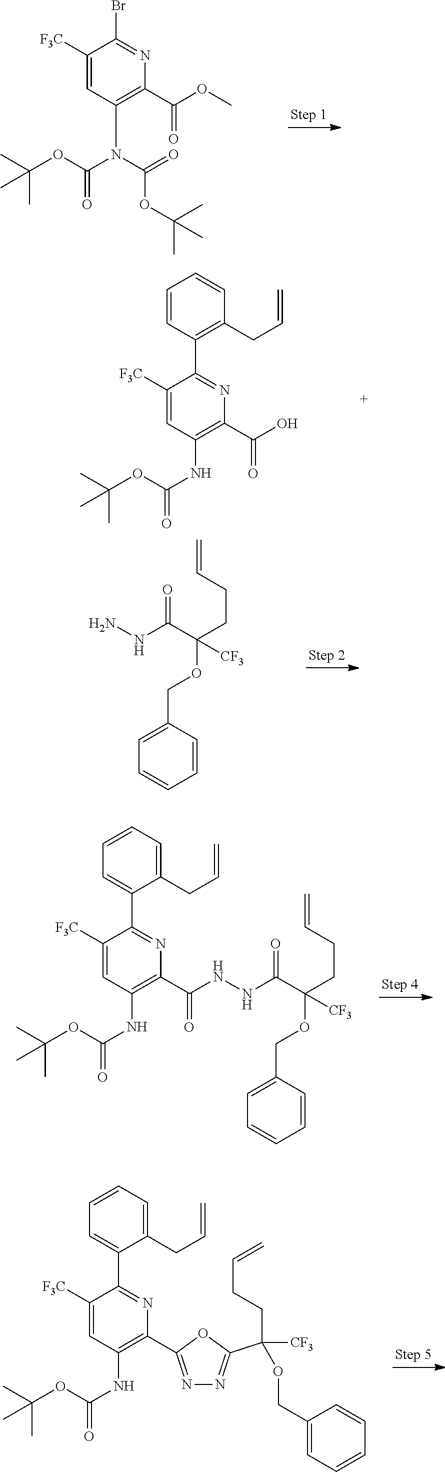

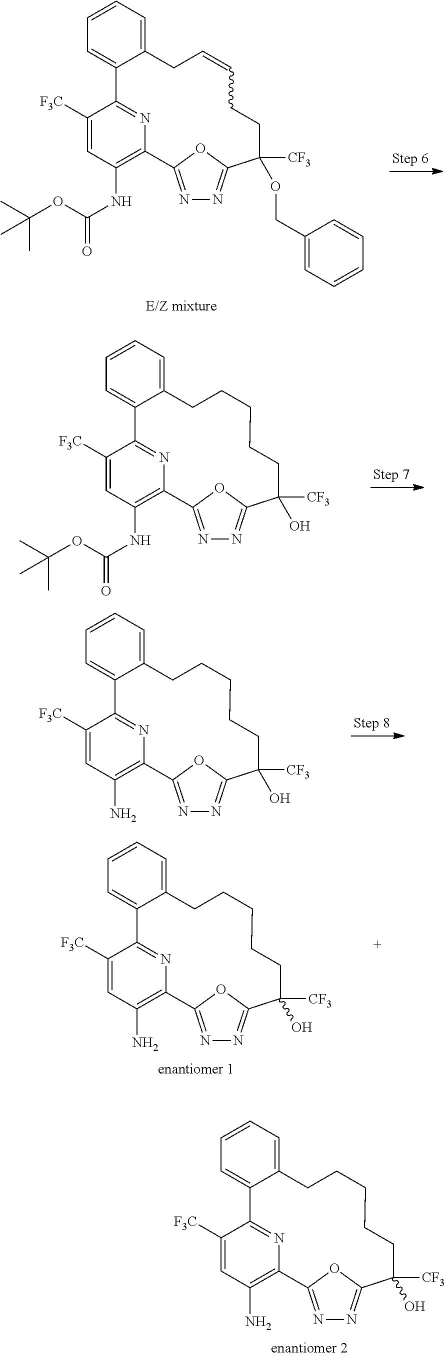

### Step 1: 6-(2-Allylphenyl)-3-(tert-butoxycarbonylamino)-5-(trifluoromethyl)pyridine-2-carboxylic Acid

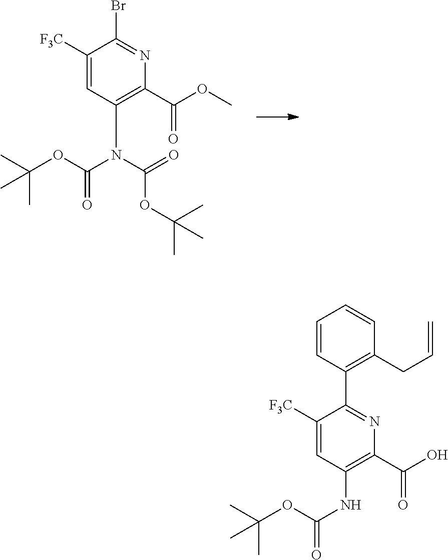 [SMILES: COC(=O)c1nc(Br)c(C(F)(F)F)cc1N(C(=O)OC(C)(C)C)C(=O)OC(C)(C)C ; C=CCc1ccccc1-c1nc(C(=O)O)c(NC(=O)OC(C)(C)C)cc1C(F)(F)F]

Part 1: In a 250-mL sealed vial methyl 3-[bis(tert-butoxycarbonyl)amino]-6-bromo-5-(trifluoromethyl)pyridine-2-carboxylate (1.0 g, 2.003 mmol) and 2-(2-allylphenyl)-4,4,5,5-tetramethyl-1,3,2-dioxaborolane (783 mg, 3.207 mmol) were combined in DMSO (10 mL). Added to the mixture were Pd(dppf)Cl₂ (211 mg, 0.2884 mmol) and potassium carbonate (1.4 g, 10.13 mmol) and nitrogen was bubbled through the suspension for 1 minute. The reaction was capped and heated at 100° C. for 20 hours. The mixture was cooled to room temperature, diluted with brine and extracted with ethyl acetate. The combined organic extracts were dried over sodium sulfate, filtered and concentrated. The resulting brown oil was purified by silica gel chromatography using 100% hexanes to 50% ethyl acetate in hexanes which gave as a brown oil, methyl 6-(2-allylphenyl)-3-[bis(tert-butoxycarbonyl)amino]-5-(trifluoromethyl)pyridine-2-carboxylate, ESI-MS m/z calc. 536.21344, found 537.2 (M+1)⁺; Retention time: 1.77 minutes (LC Method J).

Part 2: The residue was dissolved in THF (10 mL), MeOH (10 mL) and water (10 mL) followed by lithium hydroxide (340 mg, 8.102 mmol) was added. The mixture was stirred with heating at 60° C. for 3 hours. THE and methanol were removed under reduced pressure and 10 mL HCl (10%) was added (pH ˜3). The mixture was extracted with ethyl acetate (2×50 mL), combined organics dried over sodium sulfate and concentrated in vacuo to give as a tan solid, 6-(2-allylphenyl)-3-(tert-butoxycarbonylamino)-5-(trifluoromethyl)pyridine-2-carboxylic acid (785 mg, 93%), ESI-MS m/z calc. 422.14536, found 423.2 (M+1)⁺; Retention time: 1.71 minutes (LC Method J).

### Step 2: tert-Butyl N-[6-(2-allylphenyl)-2-[[[2-benzyloxy-2-(trifluoromethyl)hex-5-enoyl]amino]carbamoyl]-5-(trifluoromethyl)-3-pyridyl]carbamate

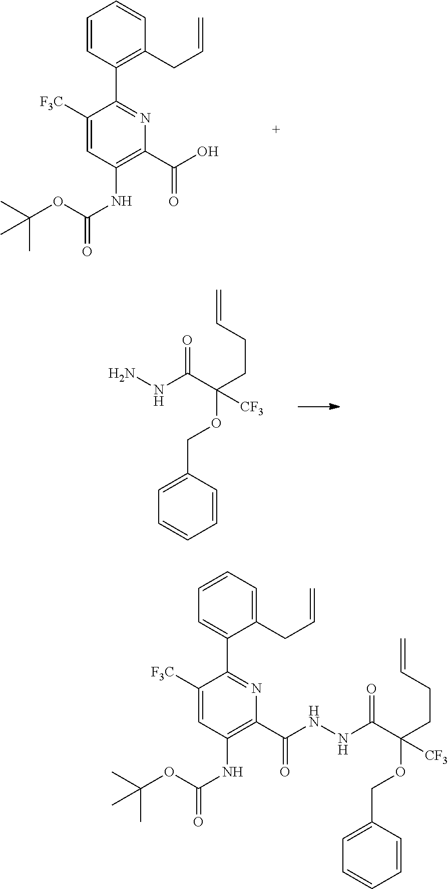 [SMILES: C=CCc1ccccc1-c1nc(C(=O)O)c(NC(=O)OC(C)(C)C)cc1C(F)(F)F ; C=CCCC(OCc1ccccc1)(C(=O)NN)C(F)(F)F ; C=CCCC(OCc1ccccc1)(C(=O)NNC(=O)c1nc(-c2ccccc2CC=C)c(C(F)(F)F)cc1NC(=O)OC(C)(C)C)C(F)(F)F]

To a solution of 6-(2-allylphenyl)-3-(tert-butoxycarbonylamino)-5-(trifluoromethyl)pyridine-2-carboxylic acid (438 mg, 1.037 mmol) in NMP (6 mL) was added 2-benzyloxy-2-(trifluoromethyl)hex-5-enehydrazide (330 mg, 1.092 mmol), DIEA (600 μL, 3.445 mmol), followed by HATU (475 mg, 1.249 mmol). The reaction mixture was stirred at room temperature for 2 h. The reaction was diluted with ethyl acetate and washed with a saturated aqueous sodium bicarbonate solution. The organic layer was further washed with 10% citric acid solution followed by brine. The organics were separated, dried over sodium sulfate, and evaporated. The residue was purified by silica gel chromatography (80 gram column) using 100% hexanes to 60% ethyl acetate in hexanes (product elutes at 18% ethyl acetate) to afford as a white foam, tert-butyl N-[6-(2-allylphenyl)-2-[[[2-benzyloxy-2-(trifluoromethyl)hex-5-enoyl]amino]carbamoyl]-5-(trifluoromethyl)-3-pyridyl]carbamate (609 mg, 83%), ESI-MS m/z calc. 706.259, found 707.2 (M+1)⁺; Retention time: 1.86 minutes (LC Method M).

### Step 3: tert-Butyl N-[6-(2-allylphenyl)-2-[5-[1-benzyloxy-1-(trifluoromethyl)pent-4-enyl]-1,3,4-oxadiazol-2-yl]-5-(trifluoromethyl)-3-pyridyl]carbamate

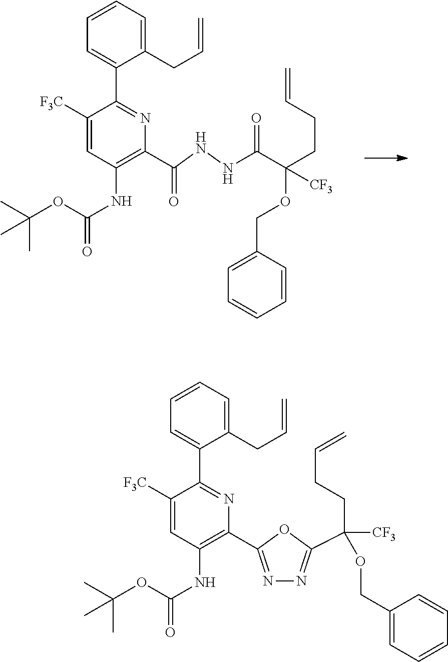 [SMILES: C=CCCC(OCc1ccccc1)(C(=O)NNC(=O)c1nc(-c2ccccc2CC=C)c(C(F)(F)F)cc1NC(=O)OC(C)(C)C)C(F)(F)F ; C=CCCC(OCc1ccccc1)(c1nnc(-c2nc(-c3ccccc3CC=C)c(C(F)(F)F)cc2NC(=O)OC(C)(C)C)o1)C(F)(F)F]

A solution of tert-butyl N-[6-(2-allylphenyl)-2-[[[2-benzyloxy-2-(trifluoromethyl)hex-5-enoyl]amino]carbamoyl]-5-(trifluoromethyl)-3-pyridyl]carbamate (605 mg, 0.8561 mmol) and DIEA (500 μL, 2.871 mmol) in acetonitrile (19 mL) was heated to 50° C., then p-toluenesulfonyl chloride (250 mg, 1.311 mmol) was added in 1 portion. The resulted mixture was heated at 70° C. for 2 hours. Reaction mixture was cooled and quenched with saturated solution of sodium bicarbonate (20 mL) and stirred for 15 minutes. The organic material was extracted with ethyl acetate (3×10 mL). The combined organics were dried over sodium sulfate and concentrated. The residue was purified by silica gel chromatography (80 gram column) using 100% hexanes to 50% ethyl acetate in hexanes to afford a tan residue which was placed under high vac pump for 18 hours which gave as a colorless oil, tert-butyl N-[6-(2-allylphenyl)-2-[5-[1-benzyloxy-1-(trifluoromethyl)pent-4-enyl]-1,3,4-oxadiazol-2-yl]-5-(trifluoromethyl)-3-pyridyl]carbamate (574 mg, 97%). ESI-MS m/z calc. 688.2484, found 689.2 (M+1)⁺; Retention time: 2.14 minutes (LC Method M).

### Step 4: tert-Butyl N-[6-(benzyloxy)-6,19-bis(trifluoromethyl)-23-oxa-3,4,22-triazatetracyclo[16.3.1.12,5.012,17]tricosa-1(22),2,4,9,12,14,16,18,20-nonaen-21-yl]carbamate (E/Z Mixture)

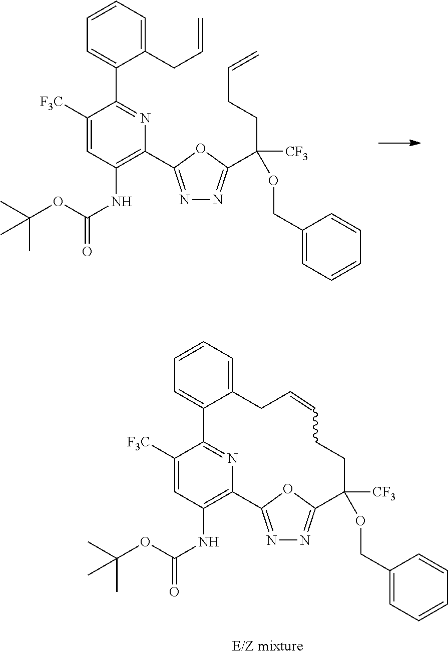 [SMILES: C=CCCC(OCc1ccccc1)(c1nnc(-c2nc(-c3ccccc3CC=C)c(C(F)(F)F)cc2NC(=O)OC(C)(C)C)o1)C(F)(F)F ; CC(C)(C)OC(=O)Nc1cc(C(F)(F)F)c2nc1-c1nnc(o1)C(OCc1ccccc1)(C(F)(F)F)CCC=CCc1ccccc1-2]

In a 500 mL round-bottom flask, a degassed solution of tert-butyl N-[6-(2-allylphenyl)-2-[5-[1-benzyloxy-1-(trifluoromethyl)pent-4-enyl]-1,3,4-oxadiazol-2-yl]-5-(trifluoromethyl)-3-pyridyl]carbamate (574 mg, 0.8335 mmol) in 1,2-dichloroethane (145 mL) was heated to 50° C. under nitrogen atmosphere. Then, dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (98 mg, 0.1336 mmol) was added in two portions over 10 minutes. The resulting mixture was heated at 70° C. for 2 hours. Another portion of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (98 mg, 0.1336 mmol) was added and the mixture was heated at 70° C. for 1 more hour. The reaction mixture was cooled to room temperature and concentrated under reduced pressure. The residue was purified by silica gel chromatography (80 gram column) using 100% hexanes to 40% ethyl acetate in hexanes (product elutes at 15% ethyl acetate) to afford a light pink residue. A second purification by silica gel chromatography (40 gram column) using 100% hexanes to 20% ethyl acetate in hexanes afforded a pale pink oil which was placed under high vac pump for 18 hours to produce a foam, tert-butyl N-[6-(benzyloxy)-6,19-bis(trifluoromethyl)-23-oxa-3,4,22-triazatetracyclo[16.3.1.12,5.012,17]tricosa-1(22),2,4,9,12,14,16,18,20-nonaen-21-yl]carbamate (E/Z mixture) (189 mg, 34%). ESI-MS m/z calc. 660.2171, found 661.2 (M+1)⁺; Retention time: 2.0 minutes (LC Method M).

### Step 5: tert-Butyl N-[6-hydroxy-6,19-bis(trifluoromethyl)-23-oxa-3,4,22-triazatetracyclo[16.3.1.12,5.012,17]tricosa-1(22),2,4,12,14,16,18,20-octaen-21-yl]carbamate

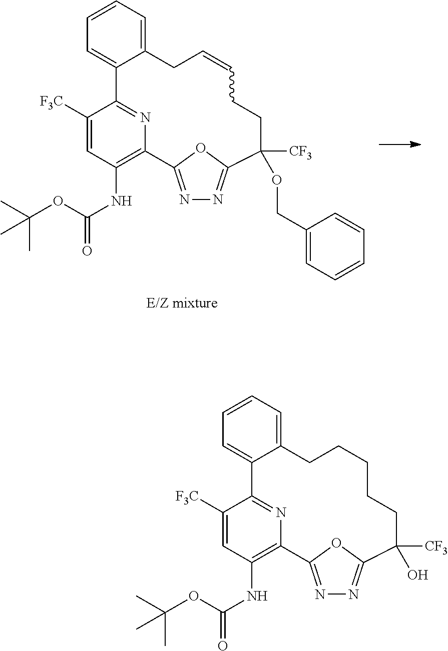 [SMILES: CC(C)(C)OC(=O)Nc1cc(C(F)(F)F)c2nc1-c1nnc(o1)C(OCc1ccccc1)(C(F)(F)F)CCC=CCc1ccccc1-2 ; CC(C)(C)OC(=O)Nc1cc(C(F)(F)F)c2nc1C1=NN=C(O1)C(O)(C(F)(F)F)CCCCCc1ccccc1-2]

To a solution of tert-butyl N-[6-(benzyloxy)-6,19-bis(trifluoromethyl)-23-oxa-3,4,22-triazatetracyclo[16.3.1.12,5.012,17]tricosa-1(22),2,4,9,12,14,16,18,20-nonaen-21-yl]carbamate (E/Z mixture) (178 mg, 0.2694 mmol) in acetic acid (2 mL) and ethyl acetate (8 mL) was added Pd/C (60 mg of 10% w/w, 0.05638 mmol). The mixture was put in a Parr Shaker and degassed under vacuum and filled with nitrogen gas three times. Then, all nitrogen gas was removed, and the reactor was pressurized to 60 psi with hydrogen gas. The mixture was shaken for 14 h. After that time, the reactor was depressurized, and additional Pd/C (180 mg of 10% w/w, 0.16 mmol) was added and the reactor was pressurized to 60 psi with hydrogen gas. The mixture was shaken for 3 more hours. The reactor was depressurized and added additional Pd/C (300 mg of 10% w/w, 0.28 mmol) and the reactor was pressurized to 60 psi with hydrogen gas. The mixture was shaken for 2 h. The reaction was filtered and concentrated under vacuum. The residue was purified by silica gel chromatography (24 gram column) using a gradient from 100% hexanes to 60% ethyl acetate in hexanes (product elutes at 20% ethyl acetate) which gave as an off-white solid, tert-butyl N-[6-hydroxy-6,19-bis(trifluoromethyl)-23-oxa-3,4,22-triazatetracyclo[16.3.1.12,5.012,17]tricosa-1(22),2,4,12,14,16,18,20-octaen-21-yl]carbamate (112.8 mg, 73%). ¹H NMR (400 MHz, DMSO-d₆) δ 9.75 (s, 1H), 8.94 (s, 1H), 7.76 (s, 1H), 7.44-7.38 (m, 2H), 7.30 (m, 1H), 2.42-1.94 (m, 6H), 1.68 (dd, J=16.0, 6.5 Hz, 1H), 1.51 (s, 9H), 1.31 (d, J=22.3 Hz, 2H), 1.23 (s, 2H) ppm. ESI-MS m/z calc. 572.18585, found 573.1 (M+1)⁺; Retention time: 1.48 minutes (LC Method M).

### Step 6: 21-Amino-6,19-bis(trifluoromethyl)-23-oxa-3,4,22-triazatetracyclo[16.3.1.12,5.012,17]tricosa-1(22),2,4,12,14,16,18,20-octaen-6-ol

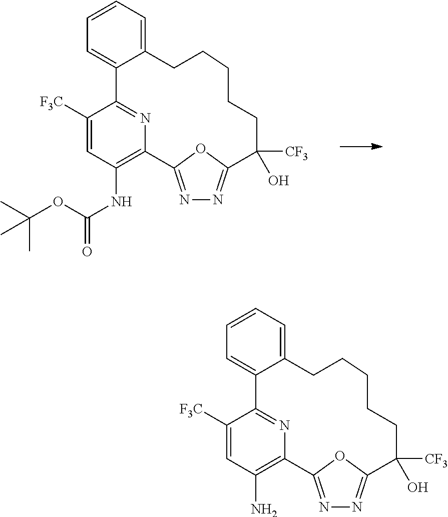 [SMILES: CC(C)(C)OC(=O)Nc1cc(C(F)(F)F)c2nc1-c1nnc(o1)C(O)(C(F)(F)F)CCCCCc1ccccc1-2 ; Nc1cc(C(F)(F)F)c2nc1-c1nnc(o1)C(O)(C(F)(F)F)CCCCCc1ccccc1-2]

tert-Butyl N-[6-hydroxy-6,19-bis(trifluoromethyl)-23-oxa-3,4,22-triazatetracyclo[16.3.1.12,5.012,17]tricosa-1(22),2,4,12,14,16,18,20-octaen-21-yl]carbamate (110 mg, 0.1921 mmol) was dissolved in dichloromethane (2 mL) and to the mixture was added TFA (1000 μL, 12.98 mmol) and stirred at room temperature for 2 hours. The mixture was evaporated, and the residue was purified by silica gel chromatography (12 gram column) using 100% hexanes to 60% ethyl acetate in hexanes (product elutes at 30% ethyl acetate). The material was further purified by reverse phase HPLC using a gradient from 1% to 99% acetonitrile in water (+5 mM HCl) over 15 minutes which gave as a white solid, 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (19.7 mg, 22%). ESI-MS m/z calc. 472.1334, found 473.2 (M+1)⁺; Retention time: 1.48 minutes (LC Method J).

### Step 7: 21-Amino-6,19-bis(trifluoromethyl)-23-oxa-3,4,22-triazatetracyclo[16.3.1.12,5.012,17]tricosa-1(22),2,4,12,14,16,18,20-octaen-6-ol (enantiomer 1) (Compound 4) and 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (Enantiomer 2) (Compound 5)

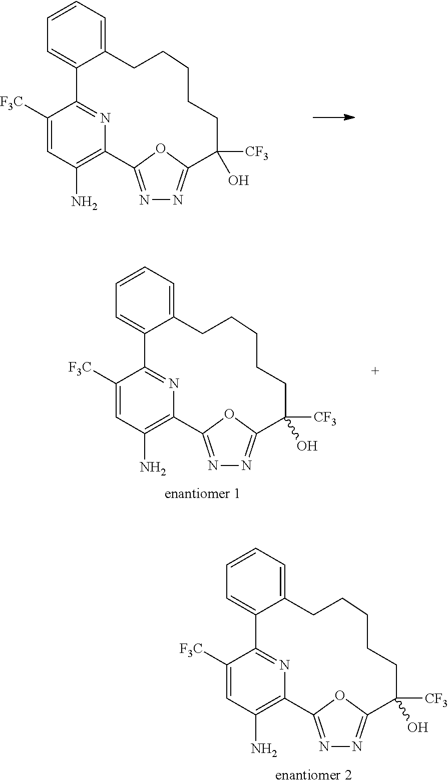 [SMILES: Nc1cc(C(F)(F)F)c2nc1-c1nnc(o1)C(O)(C(F)(F)F)CCCCCc1ccccc1-2 ; Nc1cc(C(F)(F)F)c2nc1-c1nnc(o1)C(O)(C(F)(F)F)CCCCCc1ccccc1-2 ; Nc1cc(C(F)(F)F)c2nc1-c1nnc(o1)C(O)(C(F)(F)F)CCCCCc1ccccc1-2]

Racemic 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (19.6 mg, 0.04149 mmol) was purified by chiral SFC using a Chiral Pak AS column (250×10 mm, 5 μm particle size), 10% MeOH (no modifier) and 90% CO₂ as a gradient using a flow rate 70 mL/min with an injection volume of ˜23 mg/ml in methanol (no modifier) which gave as a white solid and the first eluting enantiomer, 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (enantiomer 1) (4.7 mg, 47%). ¹H NMR (400 MHz, methanol-d4) δ 7.77 (s, 1H), 7.39 (d, J=7.7 Hz, 1H), 7.34 (d, J=4.1 Hz, 2H), 7.21 (m, 1H), 2.51-2.39 (m, 1H), 2.35 (q, J=6.9 Hz, 1H), 2.23-2.07 (m, 2H), 1.75 (m, 1H), 1.72-1.51 (m, 2H), 1.49-1.12 (m, 2H), 0.98-0.74 (m, 1H) ppm. ESI-MS m/z calc. 472.1334, found 473.2 (M+1)⁺; Retention time: 2.05 minutes (LC Method A). The later eluting enantiomer was further purified by reverse-phase preparative HPLC using a gradient from 1% to 99% acetonitrile in water (+5 mM HCl) over 15 minutes which gave as a white solid, 21-amino-6,19-bis(trifluoromethyl)-23-oxa-3,4,22-triazatetracyclo[16.3.1.12,5.012,17]tricosa-1(22),2,4,12,14,16,18,20-octaen-6-ol (enantiomer 2) (4.6 mg, 46%) ESI-MS m/z calc. 472.1334, found 473.2 (M+1)⁺; Retention time: 2.05 minutes (LC Method A).

### Example 3: Preparation of (6R)-17-amino-6-hydroxy-6,15-bis(trifluoromethyl)-19-oxa-3,4,18-triazatricyclo[12.3.1.12,5]nonadeca-1(18),2,4,14,16-pentaen-13-one (Compound 6)

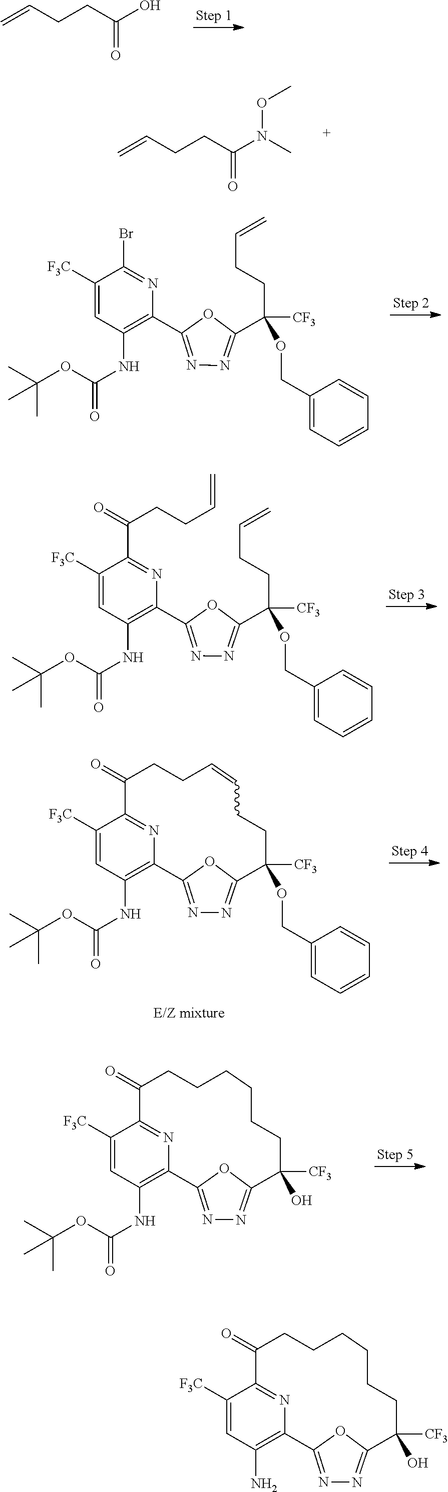

### Step 1: N-Methoxy-N-methyl-pent-4-enamide

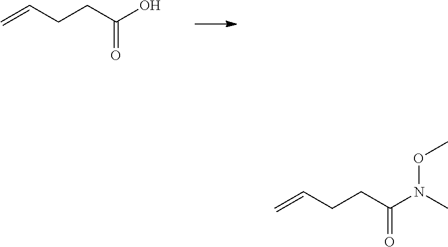

Carbonyl diimidazole (142 g, 875.7 mmol) was added to a solution of pent-4-enoic acid (71.5 mL, 700.6 mmol) in dichloromethane (2.45 L) under nitrogen, in an ice-water bath (gas evolution occurred over 34 minutes). After 1 h, added N-methoxymethanamine (hydrochloride salt) (168 g, 1.722 mol) over 3 min and then stirred for 16 h allowing the ice-water bath to eventually reach room temperature. Added a solution of citric acid (169 g, 879.6 mmol) in 1 L of ice water; temperature of the mixture dropped to 15° C. Separated the layers, washed the organic phase with HCl (700 mL of 0.25 M, 175 mmol) then a solution of potassium carbonate (24.3 g, 175.8 mmol) in 200 mL water and finally with brine. Dried the organic phase with magnesium sulfate, filtered and concentrated by rotary evaporation at 42° C. and 15 torr, then dissolved in MTBE and concentrated again giving 90.88 grams of a barely yellow liquid which was distilled (product distilled at bath temperature of 145° C., head temperature of 80° C.) providing N-methoxy-N-methyl-pent-4-enamide (81.0 g, 81%). ¹H NMR (400 MHz, Chloroform-d) δ 5.94-5.79 (m, 1H), 5.13-5.03 (m, 1H), 5.03-4.95 (m, 1H), 3.69 (s, 3H), 3.19 (s, 3H), 2.60-2.47 (m, 2H), 2.44-2.34 (m, 2H) ppm.

### Step 2: tert-Butyl N-[2-[5-[(1R)-1-benzyloxy-1-(trifluoromethyl)pent-4-enyl]-1,3,4-oxadiazol-2-yl]-6-pent-4-enoyl-5-(trifluoromethyl)-3-pyridyl]carbamate

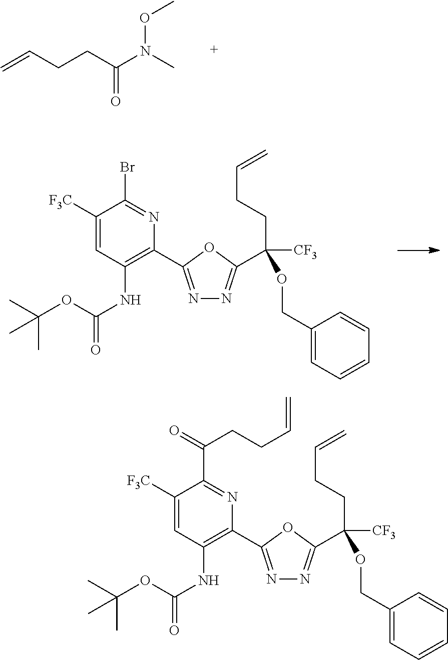

Suspended NaH (3.76 g, 94.01 mmol) in THF (250 mL) at room temperature under nitrogen. Added a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (51.0 g, 78.29 mmol) in THF (750 mL) then heated at 37° C. for 2 h. Cooled to −78° C., n-BuLi (38.8 mL of 2.5 M, 97 mmol) over 2 minutes, keeping the temperature below −65° C. Added a solution of N-methoxy-N-methyl-pent-4-enamide (14.0 g, 97.78 mmol) in THF (38 mL). Stirred at −78° C. for 5 minutes, then placed the reaction mixture in a room temperature water bath until the internal temperature reached 0° C., then maintained 0° C. for 5 minutes before quenching with acetic acid (22.3 mL, 392.1 mmol) followed by careful and slow addition of 40 mL water (gas evolution). Evaporated most of the solvent by rotary evaporation at 38° C. Added 750 mL hexanes, then washed with 500 mL water. Separated the layers, washed the organic phase with 100 mL brine, dried with magnesium sulfate, filtered and concentrated by rotary evaporation at 47° C. giving 52.1 g of a crude red oil. Purification by silica gel chromatography (column volume=4.8 L, flow rate=900 mL/min). Hexanes was used as the “A” solvent. Mixed 1.6 L of ethyl acetate with 18.4 L of hexanes to make 20 L of 8% ethyl acetate/hexanes and used this as the “B” solvent. Dissolved the 52.1 g crude in 150 mL of hexanes and ran 100% hexanes for 10 min then programmed an initial gradient to run 10% to 25% solvent B over 107 minutes, 20 column volumes, desired product eluted at 18%) which afforded tert-butyl N-[2-[5-[(1R)-1-benzyloxy-1-(trifluoromethyl)pent-4-enyl]-1,3,4-oxadiazol-2-yl]-6-pent-4-enoyl-5-(trifluoromethyl)-3-pyridyl]carbamate (22.15 g, 43%). ¹H NMR (400 MHz, Chloroform-d) δ 10.33 (s, 1H), 9.42 (s, 1H), 7.43-7.37 (m, 2H), 7.37-7.24 (m, 3H), 5.95-5.71 (m, 2H), 5.12-5.02 (m, 2H), 5.02-4.95 (m, 2H), 4.85 (d, J=10.8 Hz, 1H), 4.70 (d, J=10.8 Hz, 1H), 3.23 (t, J=7.3 Hz, 2H), 2.60-2.19 (m, 6H), 1.58 (s, 9H) ppm. ESI-MS m/z calc. 654.22766, found 655.1 (M+1)⁺; Retention time: 3.84 minutes (LC Method FF).

### Step 3: tert-Butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z Mixture)

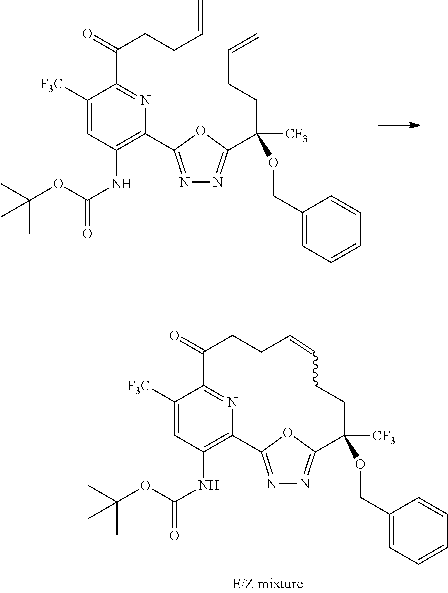 [SMILES: C=CCCC(=O)c1nc(-c2nnc([C@@](CCC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)[C@@](OCc1ccccc1)(C(F)(F)F)CCC=CCCC2=O]

Nitrogen was bubbled into a solution of tert-butyl N-[2-[5-[(1R)-1-benzyloxy-1-(trifluoromethyl)pent-4-enyl]-1,3,4-oxadiazol-2-yl]-6-pent-4-enoyl-5-(trifluoromethyl)-3-pyridyl]carbamate (36.0 g, 53.76 mmol) in toluene (4930 mL) for 30 minutes in a 12 L flask. dichloro-[(2-isopropoxyphenyl)methylene]ruthenium; tricyclohexylphosphine (3.31 g, 5.511 mmol) was added, nitrogen was bubbled directly into the mixture while it was heated to a target temperature of 108° C., but the nitrogen bubbling was stopped once the temperature reached 50° C. After reaching 108° C. the mixture was stirred for 229 min then dichloro-[(2-isopropoxyphenyl)methylene]ruthenium; tricyclohexylphosphine (1.62 g, 2.697 mmol) was added and stirring at 108° C. was continued for 17 h 40 min. Cooled to 55° C., added 2-sulfanylpyridine-3-carboxylic acid (3.76 g, 24.23 mmol), followed by triethylamine (3.38 mL, 24.25 mmol), then heated at 55° C. and stirred for 3.5 h. Concentrated the mixture by rotary evaporation at 45° C. down to a volume of roughly 750 mL, then let sit overnight at room temperature. Filtered out the insoluble precipitate and rinsed the precipitate well with hexanes (desired product is soluble in pure hexanes). Removed all solvent by rotary evaporation at 45° C. Added 700 mL of hexanes and swirled at 45° C. to help dissolve the desired product. Cooled to room temperature, added 500 mL of saturated sodium bicarbonate/H₂O 1:1 and stirred for 10 minutes then filtered and rinsed well with hexanes. Separated the layers of the filtrate, dried the organic phase with magnesium sulfate, filtered and concentrated at 48° C. giving 31.95 g of crude, darkly colored residue. The 31.95 g crude was dissolved in 100 mL MeOH and heated in a 40° C. bath (necessary to help dissolve) then 25 mL of DMSO was added to help keep the material dissolved at room temperature. The solution was loaded onto a 1.9 kg Cis reverse-phase column (column volume=1656 mL, flow rate=260 mL/min). A gradient of 65% to 93% acetonitrile/H₂O was run over 96 min (15 column volumes) then 93% acetonitrile/H₂O was maintained as an isocratic eluent for about 10 minutes then increased to 100% acetonitrile (after 140 min, the flow rate was increased to 450 mL/min to fully elute oligomeric side products) providing tert-butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (17.35 g, 50%). ESI-MS m/z calc. 626.1964, found 627.1 (M+1)⁺; Retention time: 3.67 minutes (LC Method FF).

### Step 4: tert-Butyl N-[(6R)-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

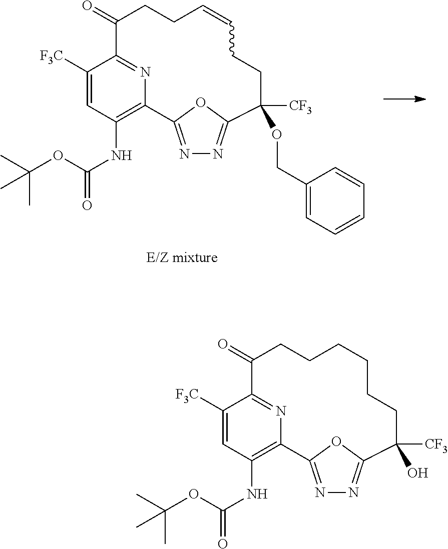 [SMILES: CC(C)(C)OC(=O)Nc1cc(C(F)(F)F)c2nc1C1=NN=C(O1)[C@@](OCc1ccccc1)(C(F)(F)F)CCC=CCCC2=O ; CC(C)(C)OC(=O)Nc1cc(C(F)(F)F)c2nc1C1=NN=C(O1)[C@@](O)(C(F)(F)F)CCCCCCC2=O]

(This procedure was conducted in two batches, two separate pots prior to combining for purification.)

Reactor 1: tert-Butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (2.0 g, 3.160 mmol) was dissolved in acetic acid (40 mL) (in a 500 mL flask), flushed with nitrogen, treated with Pd/C (882 mg of 10% w/w as 50% water wet material, 0.414 mmol), then stirred under hydrogen at room temperature for 5 h 15 min. Evacuated the flask, backfilled with nitrogen, repeated many times then let the reaction mixture sit overnight with no stirring. Analysis showed that the reaction still progressed overnight. The mixture was put back under hydrogen atmosphere for 35 minutes then evacuated and backfilled with nitrogen multiple times. Filtered the mixture through Celite, washed the Celite with ethyl acetate and concentrated the filtrate by rotary evaporation at 45° C. to give crude material which was combined with the material obtained from reactor 2 prior to purification.

Reactor 2: tert-Butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (15.6 g, 24.65 mmol) was dissolved in acetic acid (300 mL) (in a 5 L flask), flushed with nitrogen, treated with Pd/C (6.88 g of 10% w/w as 50% water wet material, 3.24 mmol), then stirred under hydrogen at room temperature for 5.5 h. Evacuated the flask and backfilled with nitrogen. Filtered the mixture through Celite, washed the Celite with ethyl acetate and concentrated the filtrate by rotary evaporation at 45° C. to give 14.85 g of crude material which was added to the crude material from reactor 1 and purified together. The combined crude material was dissolved in 45 mL MeOH. The solution was loaded onto a 1.9 kg reverse phase C₁₈ column (column volume=1656 mL, flow rate=260 mL/min). A gradient of 50% to 100% acetonitrile/H₂O was run over 128 min (20 column volumes). The product started eluting at 81% acetonitrile, so the gradient was held at 81% acetonitrile until the product finished eluting which provided 11.72 g of the title compound with residual MTBE present. This material was dissolved in a small amount of dichloromethane, then heptane was added and concentrated again at 47° C. to remove the residual MTBE giving tert-butyl N-[(6R)-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (11.17 g, 75%). ¹H NMR (400 MHz, DMSO-d₆) δ 9.82 (s, 1H), 9.08 (s, 1H), 7.73 (s, 1H), 3.00 (m, J=11.0, 4.7 Hz, 1H), 2.75 (m, J=11.1, 5.1 Hz, 1H), 2.27-2.13 (m, 2H), 2.12-1.97 (m, 1H), 1.85-1.71 (m, 1H), 1.71-1.43 (m, 15H) ppm. ESI-MS m/z calc. 538.1651, found 539.1 (M+1)⁺; Retention time: 3.28 minutes (LC Method FF).

### Step 5: (6R)-17-Amino-6-hydroxy-6,15-bis(trifluoromethyl)-19-oxa-3,4,18-triazatricyclo[12.3.1.12,5]nonadeca-1(18),2,4,14,16-pentaen-13-one (Compound 6)

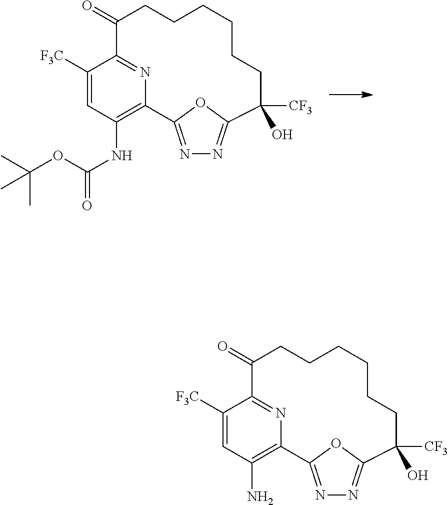 [SMILES: CC(C)(C)OC(=O)Nc1cc(C(F)(F)F)c2nc1C1=NN=C(O1)[C@@](O)(C(F)(F)F)CCCCCCC2=O ; Nc1cc(C(F)(F)F)c2nc1C1=NN=C(O1)[C@@](O)(C(F)(F)F)CCCCCCC2=O]

TFA (32 mL, 415.4 mmol) was added over 3 minutes to a solution of tert-butyl N-[(6R)-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (12.11 g, 22.49 mmol) in dichloromethane (75 mL) at 5° C. in a cold-water bath. The cold-water bath was removed, and the mixture was stirred at room temperature for 2 h 15 min (temperature reached 15° C. after 26 minutes). The reaction mixture was poured into a 0° C. solution of potassium bicarbonate (63.1 g, 630.3 mmol) in 500 mL water and 300 mL ethyl acetate. The layers were separated, and the organic layer was washed with a saturated sodium bicarbonate solution, dried with magnesium sulfate, filtered then concentrated. The residue was dissolved in boiling dichloromethane, then 100 mL heptane was added, resulting in a suspension. The dichloromethane was evaporated out of the suspension under vacuum then the product was collected by filtration and rinsed well with room temperature heptane. Dried the solid under vacuum with a nitrogen bleed at 40° C. over three days giving (6R)-17-amino-6-hydroxy-6,15-bis(trifluoromethyl)-19-oxa-3,4,18-triazatricyclo[12.3.1.12,5]nonadeca-1(18),2,4,14,16-pentaen-13-one (9.75 g, 96.8% ee). This material was further purified by SFC chromatography using a Phenomenex Lux-2 column (250×21.2 mm, 5 m particle size) with 16% methanol (20 mM NH₃) and 84% carbon dioxide mobile phase at 70 mL/min (injection volume=650 L of 32 mg/mL solution in methanol) giving as the first enantiomer to elute, (6R)-17-amino-6-hydroxy-6,15-bis(trifluoromethyl)-19-oxa-3,4,18-triazatricyclo[12.3.1.12,5]nonadeca-1(18),2,4,14,16-pentaen-13-one (8.80 g, 90%, >98% ee). ¹H NMR (400 MHz, DMSO-d₆) δ 7.80 (s, 1H), 7.77-7.37 (m, 3H), 2.99-2.84 (m, 1H), 2.77-2.62 (m, 1H), 2.28-2.10 (m, 2H), 2.10-1.94 (m, 1H), 1.85-1.70 (m, 1H), 1.70-1.40 (m, 6H) ppm. ESI-MS m/z calc. 438.11267, found 439.1 (M+1)⁺; Retention time: 1.76 minutes (LC Method A).

### Example 4: Preparation of (6S)-17-amino-6-hydroxy-6,15-bis(trifluoromethyl)-19-oxa-3,4,18-triazatricyclo[12.3.1.12,5]nonadeca-1(18),2,4,14,16-pentaen-13-one (Compound 7)

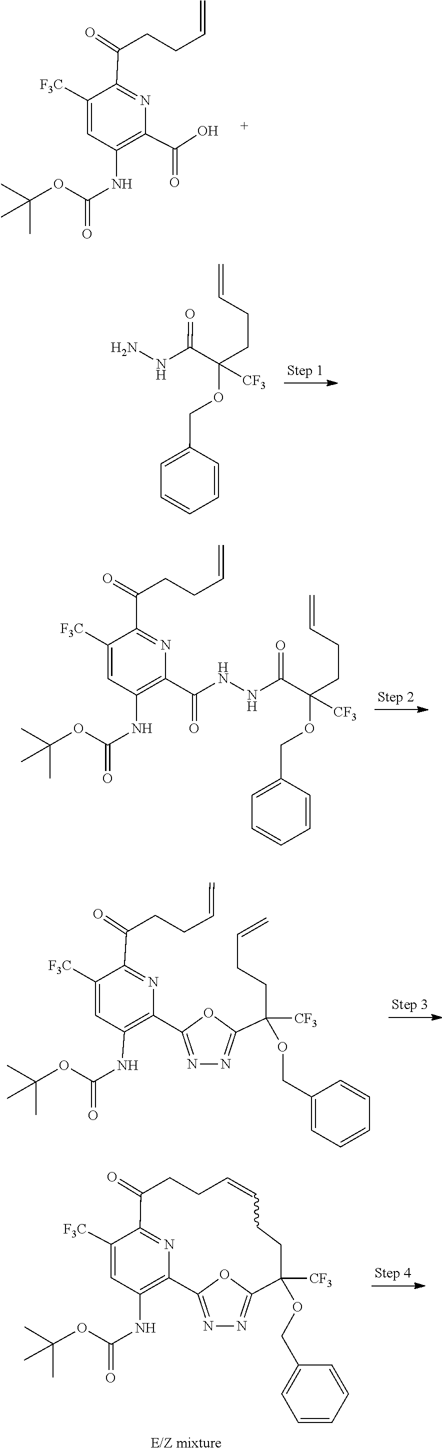

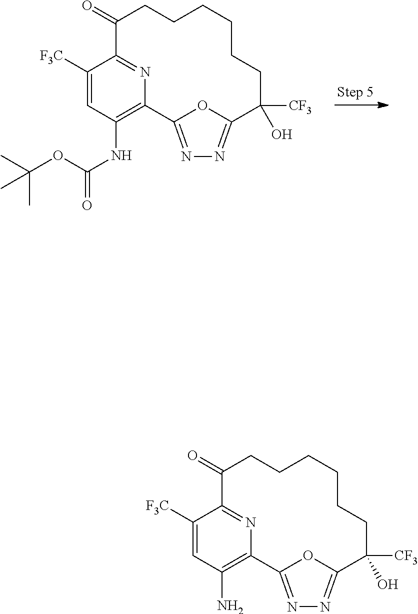 [SMILES: CC(C)(C)OC(=O)Nc1cc(C(F)(F)F)c2nc1-c1nnc(o1)C(O)(C(F)(F)F)CCCCCCC2=O ; Nc1cc(C(F)(F)F)c2nc1-c1nnc(o1)[C@](O)(C(F)(F)F)CCCCCCC2=O]

### Step 1: tert-Butyl N-[2-[[[2-benzyloxy-2-(trifluoromethyl)hex-5-enoyl]amino]carbamoyl]-6-pent-4-enoyl-5-(trifluoromethyl)-3-pyridyl]carbamate

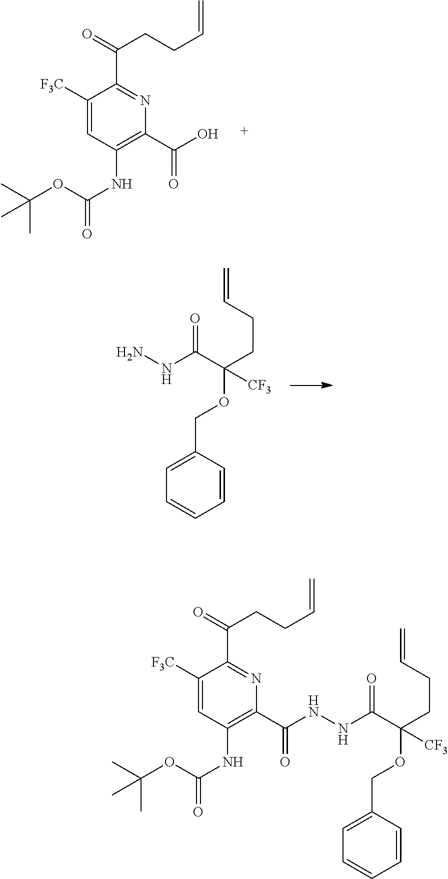 [SMILES: C=CCCC(=O)c1nc(C(=O)O)c(NC(=O)OC(C)(C)C)cc1C(F)(F)F ; C=CCCC(OCc1ccccc1)(C(=O)NN)C(F)(F)F ; C=CCCC(=O)c1nc(C(=O)NNC(=O)C(CCC=C)(OCc2ccccc2)C(F)(F)F)c(NC(=O)OC(C)(C)C)cc1C(F)(F)F]

To a solution of 3-(tert-butoxycarbonylamino)-6-pent-4-enoyl-5-(trifluoromethyl)pyridine-2-carboxylic acid (2.1 g, 5.408 mmol) in NMP (20 mL) was added DIEA (2.0 g, 15.47 mmol), followed by HATU (3.1 g, 8.153 mmol). The reaction mixture was stirred at room temperature for 2.5 h. The mixture was diluted with water (100 mL) and extracted with ethyl acetate (3×50 mL). The organic phases were combined and dried over MgSO₄, filtered, and concentrated in vacuo. The residue was purified by silica gel chromatography using 0% to 10% ethyl acetate in hexanes to give tert-butyl N-[2-[[[2-benzyloxy-2-(trifluoromethyl)hex-5-enoyl]amino]carbamoyl]-6-pent-4-enoyl-5-(trifluoromethyl)-3-pyridyl]carbamate (3.1 g, 85%). ¹H NMR (400 MHz, CDCl₃) δ 10.81 (s, 1H), 9.87 (d, J=5.2 Hz, 1H), 9.36 (s, 1H), 9.08 (d, J=5.2 Hz, 1H), 7.62-7.35 (m, 5H), 5.87 (m, 2H), 5.27-4.96 (m, 4H), 4.87 (d, J=10.5 Hz, 1H), 4.74 (d, J=10.5 Hz, 1H), 3.17 (t, J=7.2 Hz, 2H), 2.53-244 (m, 3H), 2.41-2.15 (m, 3H), 1.54 (s, 9H) ppm. ESI-MS m/z calc. 672.2383, found 673.4 (M+1)⁺; Retention time: 1.65 minutes (LC Method M).

### Step 2: tert-Butyl N-[2-[5-[1-benzyloxy-1-(trifluoromethyl)pent-4-enyl]-1,3,4-oxadiazol-2-yl]-6-pent-4-enoyl-5-(trifluoromethyl)-3-pyridyl]carbamate

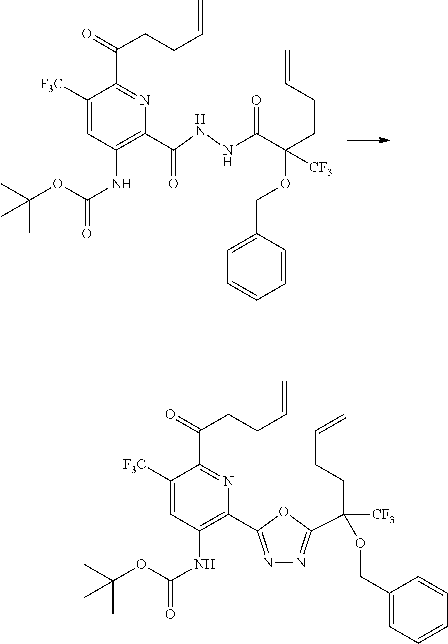 [SMILES: C=CCCC(=O)c1nc(C(=O)NNC(=O)C(CCC=C)(OCc2ccccc2)C(F)(F)F)c(NC(=O)OC(C)(C)C)cc1C(F)(F)F ; C=CCCC(=O)c1nc(-c2nnc(C(CCC=C)(OCc3ccccc3)C(F)(F)F)o2)c(NC(=O)OC(C)(C)C)cc1C(F)(F)F]

A solution of tert-butyl N-[2-[[[2-benzyloxy-2-(trifluoromethyl)hex-5-enoyl]amino]carbamoyl]-6-pent-4-enoyl-5-(trifluoromethyl)-3-pyridyl]carbamate (565 mg, 0.84 mmol) and DIEA (271 mg, 2.097 mmol) in acetonitrile (30 mL) was heated to 50° C. then tosyl chloride (192 mg, 1.007 mmol) was added. The resulting mixture was heated to 70° C. and after 90 minutes the reaction mixture was cooled and quenched with a saturated aqueous solution of sodium bicarbonate (10 mL) and then extracted with ethyl acetate (3×20 mL). The organic layers were combined and then washed with brine (30 mL), dried over sodium sulfate, filtered and concentrated to a viscous oil which was purified by silica gel chromatography eluting with 10% ethyl acetate in hexane which afforded tert-butyl N-[2-[5-[1-benzyloxy-1-(trifluoromethyl)pent-4-enyl]-1,3,4-oxadiazol-2-yl]-6-pent-4-enoyl-5-(trifluoromethyl)-3-pyridyl]carbamate (405 mg, 74%). ¹H NMR (400 MHz, Chloroform-d) δ 10.34 (s, 1H), 9.43 (s, 1H), 7.46-7.38 (m, 2H), 7.36-7.21 (m, 3H), 5.83 (m, 2H), 5.07 (m, 2H), 4.99 (m, 2H), 4.86 (d, J=10.8 Hz, 1H), 4.70 (d, J=10.8 Hz, 1H), 3.23 (t, J=7.3 Hz, 2H), 2.70-2.47 (m, 4H), 2.45-2.21 (m, 2H), 1.58 (s, 9H) ppm. ESI-MS m/z calc. 654.22766, found 655.2 (M+1)⁺; Retention time: 2.05 minutes (LC Method M).

### Step 3: tert-Butyl N-[6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z Mixture)

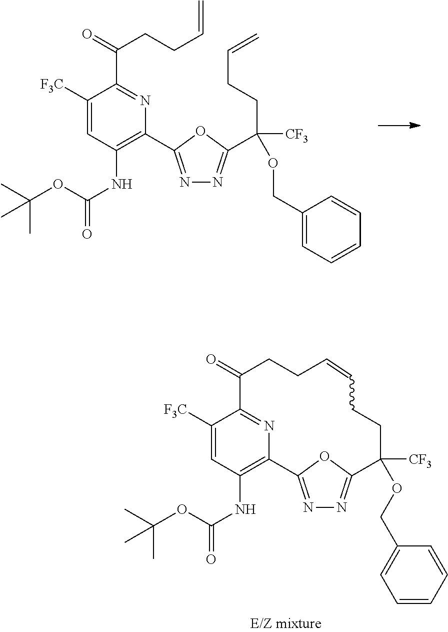 [SMILES: C=CCCC(=O)c1nc(-c2nnc(C(CCC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(OCc1ccccc1)(C(F)(F)F)CCC=CCCC2=O]

In a three necked 1 L flask, [1,3-bis-(2-tolyl)-2-imidazolidinylidene]dichloro(2-isopropoxybenzylidene)ruthenium(II) (85.6 mg, 0.1500 mmol) was dissolved in 1,2-dichloroethane (200 mL) and bubbled nitrogen gas into the solution and heated to 60° C. A solution of tert-butyl N-[2-[5-[1-benzyloxy-1-(trifluoromethyl)pent-4-enyl]-1,3,4-oxadiazol-2-yl]-6-pent-4-enoyl-5-(trifluoromethyl)-3-pyridyl]carbamate (1.25 g, 1.91 mmol) in 1,2-dichloroethane (200 mL) was added dropwise during one hour to the catalyst solution. The mixture was heated at 75° C. for 6 hours. The reaction mixture was cooled to room temperature and solvent was evaporated. The residue was purified by silica gel chromatography using hexane and ethyl acetate (85:15) and further purified by reverse phase chromatography using a gradient from 50% acetonitrile in water to 100% acetonitrile which provided tert-butyl N-[6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (815 mg, 68%). ESI-MS m/z calc. 626.1964, found 627.0 (M+1)⁺; Retention time: 1.74 minutes (LC Method M).

### Step 4: tert-Butyl N-[6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

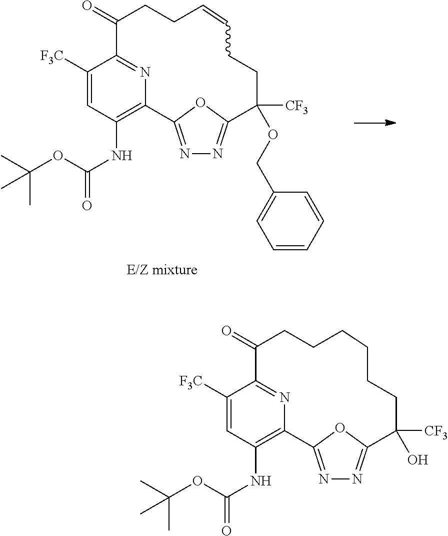 [SMILES: CC(C)(C)OC(=O)Nc1cc(C(F)(F)F)c2nc1C1=NN=C(O1)C(OCc1ccccc1)(C(F)(F)F)CCC=CCCC2=O ; CC(C)(C)OC(=O)Nc1cc(C(F)(F)F)c2nc1C1=NN=C(O1)C(O)(C(F)(F)F)CCCCCCC2=O]

To a stirred solution of tert-butyl N-[6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (220 mg, 0.3511 mmol) in acetic acid (5 mL) in a Parr reactor was added Pd/C (34 mg, 10% w/w, 50% wet, 0.03195 mmol) under nitrogen gas. The Parr reactor was filled with nitrogen gas and evacuated 3 times. Finally, the reactor was evacuated and filled with hydrogen gas up to 100 psi. The reaction was stirred at room temperature for 15 hours. The reactor was evacuated and filled by nitrogen gas and the mixture was filtered through Celite pad. The solvent was evaporated, and the residue was dissolved in dichloromethane (2 mL) and pyridinium chlorochromate (75.7 mg, 0.3512 mmol) and 76 mg Celite was added and the mixture was stirred for 2 hours. The reaction mixture was filtered through a pad of Celite and washed with ethyl acetate (10 mL). The solvent was evaporated and the residue was purified by silica gel chromatography using 80:20 of hexanes:EtOAc to afford tert-butyl N-[6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (140 mg, 74%). ESI-MS m/z calc. 538.1651, found 539.0 (M+1)⁺; Retention time: 1.72 minutes (LC Method J).

### Step 5: (6S)-17-Amino-6-hydroxy-6,15-bis(trifluoromethyl)-19-oxa-3,4,18-triazatricyclo[12.3.1.12,5]nonadeca-1(18),2,4,14,16-pentaen-13-one (Compound 7)

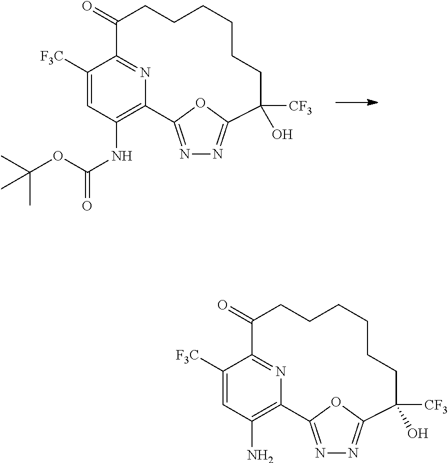 [SMILES: CC(C)(C)OC(=O)Nc1cc(C(F)(F)F)c2nc1C1=NN=C(O1)C(O)(C(F)(F)F)CCCCCCC2=O ; Nc1cc(C(F)(F)F)c2nc1C1=NN=C(O1)[C@](O)(C(F)(F)F)CCCCCCC2=O]

To a stirred solution of tert-butyl N-[6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (50 mg, 0.09286 mmol) in CH₂Cl₂ (3 mL) was added TFA (2.22 g, 19.47 mmol) at room temperature and the mixture was stirred for 45 minutes. The mixture was poured into saturated NaHCO₃ solution (5 mL) and extracted with ethyl acetate (3×10 mL). The organic layers were combined, dried, and concentrated under vacuum. The residue was purified by silica gel chromatography using 85:15 mixture of hexane:ethyl acetate. The resultant residue was further purified by chiral SFC using a ChiralCel OZ column (250×10 mm, 5 m particle size), 15% MeOH (no modifier) and 85% CO₂ as a gradient using a flow rate 10 mL/min with an injection volume of ˜24 mg/ml in methanol (no modifier) which gave as a white solid and the second eluting enantiomer (peak 2), (6S)-17-amino-6-hydroxy-6,15-bis(trifluoromethyl)-19-oxa-3,4,18-triazatricyclo[12.3.1.12,5]nonadeca-1(18),2,4,14,16-pentaen-13-one (13 mg, 63%). ¹H NMR (400 MHz, acetone-d₆) δ 7.86 (s, 1H), 7.24 (s, 2H), 6.51 (s, 1H), 3.04 (m, 1H), 2.74 (m, 1H), 2.46-2.29 (m, 2H), 2.26-2.07 (m, 1H), 1.82 (m, 3H), 1.66 (m, 4H) ppm. ESI-MS m/z calc. 438.11267, found 439.1 (M+1)⁺; Retention time: 1.21 minutes (LC Method J).

### Example 5: Preparation of 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 1) (Compound 8), 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 2) (Compound 9), 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 2, enantiomer 3) (Compound 10), and 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 2, enantiomer 4) (Compound 11)

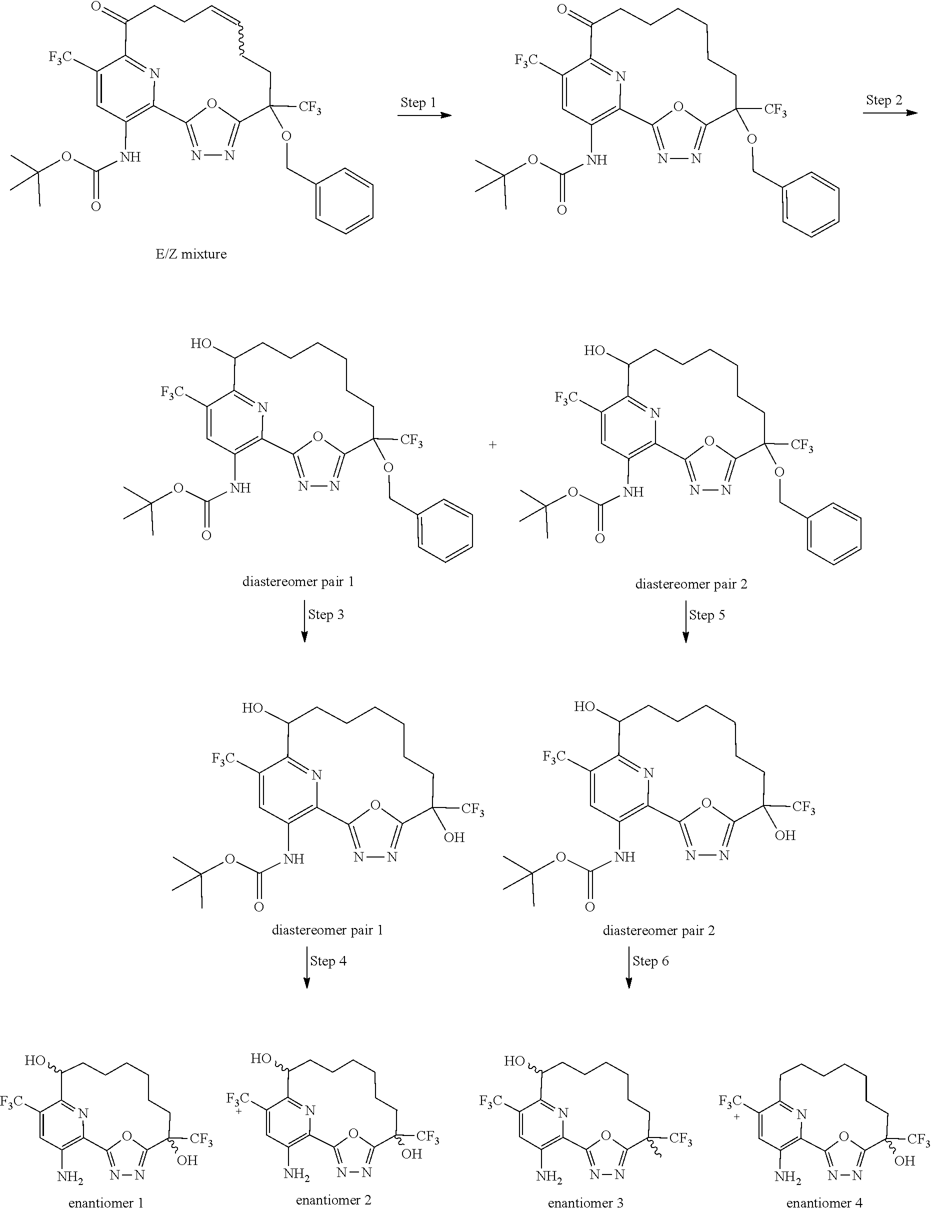

### Step 1: tert-Butyl N-[6-benzyloxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

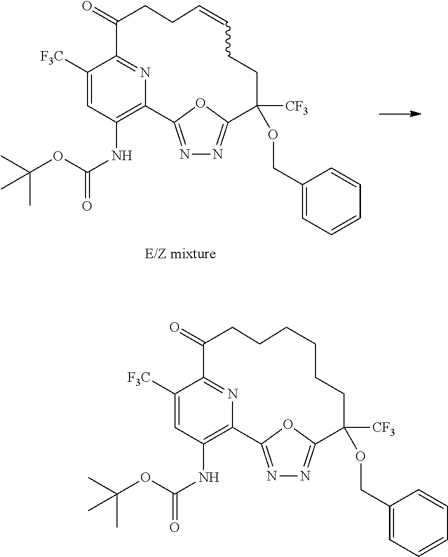 [SMILES: CC(C)(C)OC(=O)Nc1cc(C(F)(F)F)c2nc1C1=NN=C(O1)C(OCc1ccccc1)(C(F)(F)F)CCC=CCCC2=O ; CC(C)(C)OC(=O)Nc1cc(C(F)(F)F)c2nc1C1=NN=C(O1)C(OCc1ccccc1)(C(F)(F)F)CCCCCCC2=O]

To a solution of tert-butyl N-[6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (315 mg, 0.5028 mmol) in ethyl acetate (3 mL) was added Pd/C (26.8 mg of 10% w/w, 0.02518 mmol) and the mixture was evacuated and filled with nitrogen gas 3 times. Finally, the flask was evacuated and filled with hydrogen gas through a rubber septum and hydrogen balloon. The mixture was stirred vigorously for 45 min and filtered through Celite. The solvent was evaporated and the residue was purified by silica gel chromatography using 85:15 hexane/ethyl acetate giving tert-butyl N-[6-benzyloxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (245 mg, 78%). ¹H NMR (400 MHz, CDCl₃) δ 9.88 (s, 1H), 9.37 (s, 1H), 7.30-7.14 (m, 5H), 4.87 (d, J=11.3 Hz, 1H), 4.79 (d, J=11.3 Hz, 1H), 3.06 (m, 1H), 2.80 (m, 1H), 2.53 (m, 1H), 2.23 (m, 1H), 2.12 (m, 1H), 1.94 (m, 1H), 1.88-1.73 (m, 2H), 1.66 (m, 3H), 1.58 (s, 9H), 0.93-0.78 (m, 1H) ppm. ESI-MS m/z calc. 628.21204, found 629.3 (M+1)⁺; Retention time: 1.89 minutes (LC Method J).

### Step 2: tert-Butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) and tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 2)

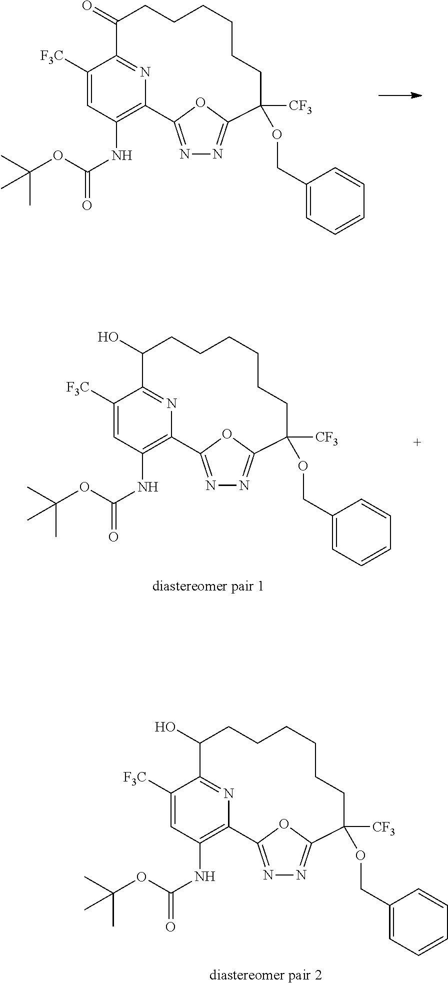 [SMILES: CC(C)(C)OC(=O)Nc1cc(C(F)(F)F)c2nc1C1=NN=C(O1)C(OCc1ccccc1)(C(F)(F)F)CCCCCCC2=O ; CC(C)(C)OC(=O)Nc1cc(C(F)(F)F)c2nc1C1=NN=C(O1)C(OCc1ccccc1)(C(F)(F)F)CCCCCCC2O ; CC(C)(C)OC(=O)Nc1cc(C(F)(F)F)c2nc1C1=NN=C(O1)C(OCc1ccccc1)(C(F)(F)F)CCCCCCC2O]

To a stirred solution of tert-butyl N-[6-benzyloxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (147 mg, 0.2339 mmol) in MeOH (5 mL) was added sodium borohydride (17.7 mg, 0.4678 mmol) at room temperature and stirred it for 15 minutes. The diastereomeric products were separated by reverse-phase chromatography using a mobile gradient from 50% water/acetonitrile to 100% acetonitrile giving as the first diastereomeric pair to co-elute, tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (67 mg, 91%). ESI-MS m/z calc. 630.22766, found 631.5 (M+1)⁺; Retention time: 2.14 minutes (LC Method J).

The second diastereomeric pair to co-elute was isolated as tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (64 mg, 87%). ESI-MS m/z calc. 630.22766, found 631.3 (M+1)⁺; Retention time: 2.21 minutes (LC Method J).

### Step 3: tert-Butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 1)

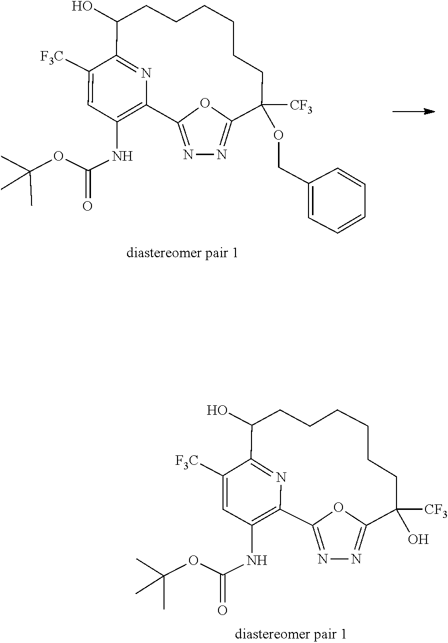 [SMILES: CC(C)(C)OC(=O)Nc1cc(C(F)(F)F)c2nc1C1=NN=C(O1)C(OCc1ccccc1)(C(F)(F)F)CCCCCCC2O ; CC(C)(C)OC(=O)Nc1cc(C(F)(F)F)c2nc1-c1nnc(o1)C(O)(C(F)(F)F)CCCCCCC2O]

To a stirred solution of tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (40 mg, 0.06343 mmol) in acetic acid (4 mL) under nitrogen gas was added Pd/C (4.8 mg, 10% w/w, 0.004510 mmol). The flask was evacuated and filled with nitrogen gas three times and finally it was filled with hydrogen gas with two hydrogen balloons through a rubber septum. The mixture was stirred for 12 hours at room temperature. The mixture was filtered through a pad of Celite and washed with ethyl acetate. The solvent was evaporated and the residue was purified by reverse phase chromatography using a mobile gradient from 50% acetonitrile in water to 100% acetonitrile providing tert-butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (21 mg, 61%). ESI-MS m/z calc. 540.1807, found 541.0 (M+1)⁺; Retention time: 1.48 minutes (LC Method J).

### Step 4: 17-Amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 1) (Compound 8) and 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 2) (Compound 9)

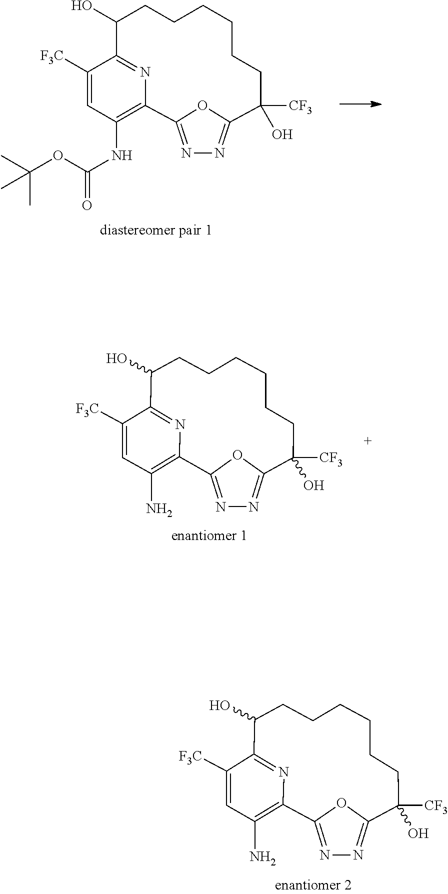 [SMILES: CC(C)(C)OC(=O)Nc1cc(C(F)(F)F)c2nc1-c1nnc(o1)C(O)(C(F)(F)F)CCCCCCC2O ; Nc1cc(C(F)(F)F)c2nc1-c1nnc(o1)C(O)(C(F)(F)F)CCCCCCC2O ; Nc1cc(C(F)(F)F)c2nc1-c1nnc(o1)C(O)(C(F)(F)F)CCCCCCC2O]

To a stirred solution of tert-butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (23 mg, 0.04256 mmol) in dichloromethane (2 mL) was added TFA (1.48 g, 12.98 mmol) at room temperature and the mixture was stirred for 15 minutes. Then a saturated solution of NaHCO₃ (2 mL) was added slowly and the product was extracted with dichloromethane (3×5 mL). Combined organic layers were combined, dried over Na₂SO₄, filtered, concentrated under vacuum and purified by reverse phase chromatography using a mobile gradient from 50% acetonitrile in water to 100% acetonitrile. Enantiomers were separated by chiral SFC using a ChiralCel OJ-3 column (250×10 mm, 5 m), 10% MeOH (20 mM NH₃) and 90% CO₂ as a gradient with an injection volume of ˜22 mg/ml in methanol (no modifier) giving as the first eluting enantiomer, 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 1) (3.1 mg, 32%). ¹H NMR (400 MHz, acetone-d₆) δ 7.76 (s, 1H), 6.51 (d, J=70.1 Hz, 2H), 4.94 (t, J=8.1 Hz, 1H), 4.12 (d, J=7.4 Hz, 1H), 2.58-2.29 (m, 2H), 2.28-2.14 (m, 1H), 1.87-1.52 (m, 9H), 1.40 (bs, 1H) ppm. ESI-MS m/z calc. 440.1283, found 441.0 (M+1)⁺; Retention time: 0.73 minutes (LC Method J).

The later eluting enantiomer provided, 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 1, enantiomer 2) (3 mg, 31%). ¹H NMR (400 MHz, acetone-d₆) δ 7.76 (s, 1H), 6.51 (d, J=70.1 Hz, 2H), 4.94 (t, J=8.1 Hz, 1H), 4.12 (d, J=7.4 Hz, 1H), 2.58-2.29 (m, 2H), 2.28-2.14 (m, 1H), 1.87-1.52 (m, 9H), 1.40 (bs, 1H) ppm. ESI-MS m/z calc. 440.1283, found 441.0 (M+1)⁺; Retention time: 0.73 minutes (LC Method J).

### Step 5: tert-Butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 2)

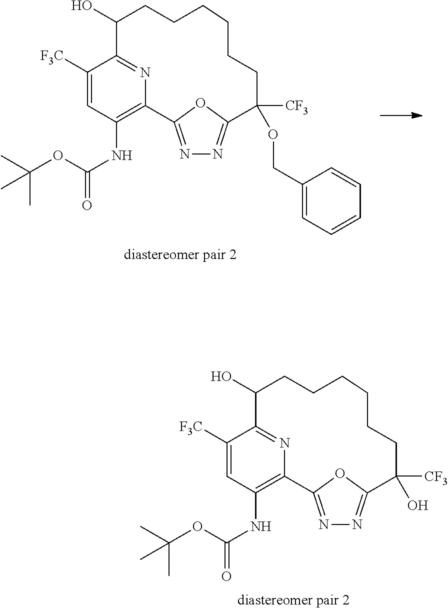 [SMILES: CC(C)(C)OC(=O)Nc1cc(C(F)(F)F)c2nc1C1=NN=C(O1)C(OCc1ccccc1)(C(F)(F)F)CCCCCCC2O ; CC(C)(C)OC(=O)Nc1cc(C(F)(F)F)c2nc1C1=NN=C(O1)C(O)(C(F)(F)F)CCCCCCC2O]

To a stirred solution of tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (40 mg, 0.06343 mmol) in acetic acid (4 mL) under nitrogen gas was added Pd/C (4.8 mg, 10% w/w, 0.004510 mmol). The flask was evacuated and filled with nitrogen gas three times and finally it was capped with two balloons of hydrogen through a rubber septum creating a hydrogen gas atmosphere. The mixture was stirred for 12 h at room temperature. The mixture was purged with nitrogen then filtered through a pad of Celite eluting with ethyl acetate. The filtrate was evaporated and the resulting residue was purified by reverse phase chromatography eluting with a gradient of 50% acetonitrile in water to 100% acetonitrile giving tert-butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (23 mg, 67%). ESI-MS m/z calc. 540.1807, found 541.0 (M+1)⁺; Retention time: 1.71 minutes (LC Method J).

### Step 6: 17-Amino-6,15-bis(trifluoromethyl)-19-oxa-3,4,18-triazatricyclo[12.3.1.12,5]nonadeca-1(18),2,4,14,16-pentaene-6,13-diol (diastereomer pair 2, enantiomer 3) (Compound 10) and 17-amino-6,15-bis(trifluoromethyl)-19-oxa-3,4,18-triazatricyclo[12.3.1.12,5]nonadeca-1(18),2,4,14,16-pentaene-6,13-diol (Diastereomer pair 2, Enantiomer 4) (Compound 11)

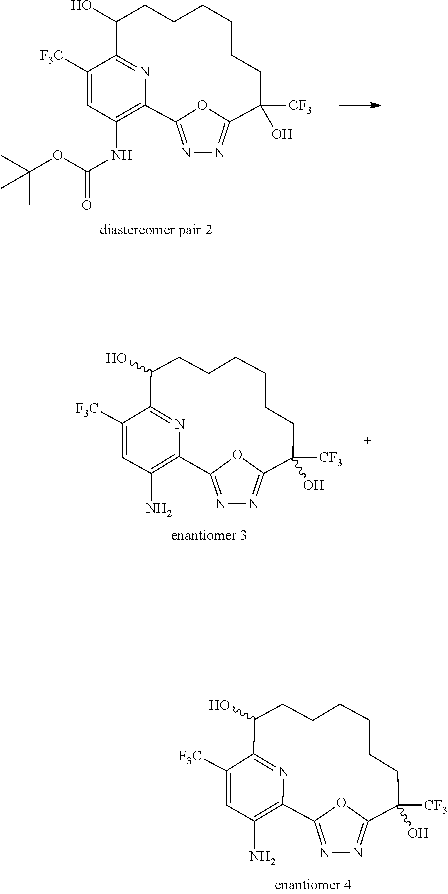 [SMILES: CC(C)(C)OC(=O)Nc1cc(C(F)(F)F)c2nc1-c1nnc(o1)C(O)(C(F)(F)F)CCCCCCC2O ; Nc1cc(C(F)(F)F)c2nc1-c1nnc(o1)C(O)(C(F)(F)F)CCCCCCC2O ; Nc1cc(C(F)(F)F)c2nc1-c1nnc(o1)C(O)(C(F)(F)F)CCCCCCC2O]

To a stirred solution of tert-butyl N-[6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (20 mg, 0.03701 mmol) in dichloromethane (2 mL) was added TFA (1.48 g, 12.98 mmol) at room temperature and the mixture was stirred for 15 min. Then, a saturated aqueous solution of sodium bicarbonate (2 mL) was added slowly and the resulting mixture was extracted with dichloromethane (3×5 mL). The organic layers were combined, dried over sodium sulfate, filtered and concentrated under vacuum. The residue was purified by reverse-phase chromatography eluting with a gradient of 50% acetonitrile in water to 100% acetonitrile. This material was further purified by chiral SFC using ChiralCel AD-3 column (250×10 mm, 5 m particle size), 15% MeOH (20 mM NH₃) and 85% CO₂ mobile phase which gave as a white solid and the first enantiomer to elute, 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 2, enantiomer 3) (3.2 mg, 38%). ¹H NMR (400 MHz, acetone-d₆) δ 7.76 (s, 1H), 6.59 (s, 2H), 6.47 (s, 1H), 5.03 (t, J=7.7 Hz, 1H), 4.10 (d, J=7.1 Hz, 1H), 2.37-2.28 (m, 3H), 1.96-1.67 (m, 4H), 1.65-1.53 (m, 5H) ppm. ESI-MS m/z calc. 440.1283, found 441.1 (M+1)⁺; Retention time: 6.94 minutes (LC Method P).

The later eluting enantiomer gave 17-amino-6,15-bis(trifluoromethyl)-19-oxa-3,4,18-triazatricyclo[12.3.1.12,5]nonadeca-1(18),2,4,14,16-pentaene-6,13-diol (diastereomer pair 2, enantiomer 4) (2.5 mg, 30%). ¹H NMR (400 MHz, acetone-d₆) δ 7.76 (s, 1H), 6.59 (s, 2H), 6.47 (s, 1H), 5.03 (t, J=7.7 Hz, 1H), 4.10 (d, J=7.1 Hz, 1H), 2.37-2.28 (m, 3H), 1.96-1.67 (m, 4H), 1.65-1.53 (m, 5H) ppm. ESI-MS m/z calc. 440.1283, found 441.1 (M+1)⁺; Retention time: 6.94 minutes (LC Method P).

### Example 6: Preparation of 17-Amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 1) (Compound 12) and 17-amino-13-methoxy-6,15-bis(trifluoromethyl)-19-oxa-3,4,18-triazatricyclo[12.3.1.12,5]nonadeca-1(18),2,4,14,16-pentaen-6-ol (Diastereomer Pair 1, Enantiomer 2) (Compound 13)

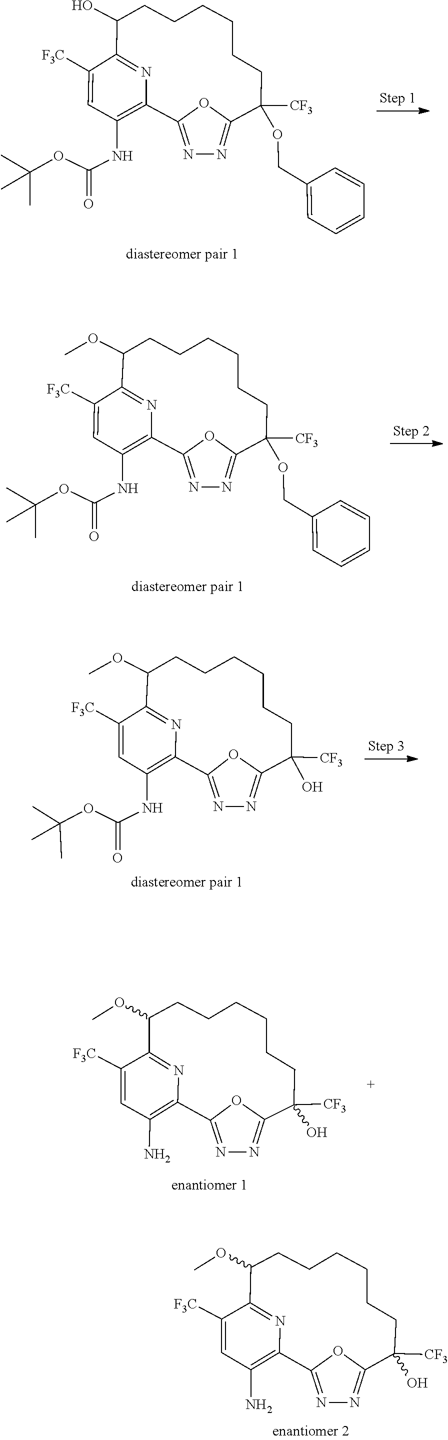 [SMILES: CC(C)(C)OC(=O)Nc1cc(C(F)(F)F)c2nc1-c1nnc(o1)C(OCc1ccccc1)(C(F)(F)F)CCCCCCC2O ; COC1CCCCCCC(OCc2ccccc2)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2N ; COC1CCCCCCC(O)(C(F)(F)F)C2=NN=C(O2)c2nc1c(C(F)(F)F)cc2N]

### Step 1: tert-Butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 1)

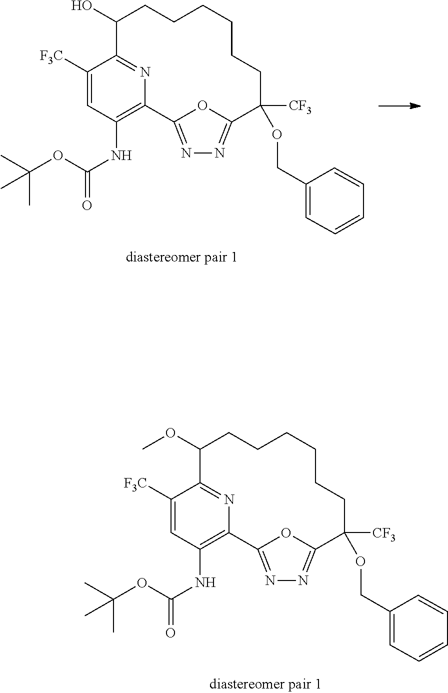 [SMILES: CC(C)(C)OC(=O)Nc1cc(C(F)(F)F)c2nc1-c1nnc(o1)C(OCc1ccccc1)(C(F)(F)F)CCCCCCC2O ; COC1CCCCCCC(OCc2ccccc2)(C(F)(F)F)c2nnc(o2)-c2nc1c(C(F)(F)F)cc2NC(=O)OC(C)(C)C]

To a stirred solution of tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (55 mg, 0.08722 mmol) in THF (4 mL) at 0° C. (ice bath) was added potassium tert-butoxide (47 mg of 25% w/w, 0.1047 mmol, 2 M solution in 2-methyl tetrahydrofuran) under an inert atmosphere. The mixture was stirred for 5 min and then methyl iodide (14.9 mg, 0.105 mmol) was added by a syringe via a rubber septum. The mixture was stirred for an additional 30 minutes. The reaction was quenched at 0° C. by adding 10 mL water and extracted with diethyl ether (3×20 mL). Organic layers were combined, dried over sodium sulfate, filtered, concentrated and purified silica gel chromatography eluting with 85:15 hexane/ethyl acetate which gave tert-butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (21 mg, 37%). ¹H NMR (400 MHz, DMSO-d₆) δ 9.67 (s, 1H), 8.98 (s, 1H), 7.61-7.08 (m, 5H), 5.04-4.52 (m, 3H), 3.35 (s, 3H), 2.78-2.50 (m, 1H), 2.38-2.09 (m, 2H), 1.89 (t, J=12.0 Hz, 1H), 1.78-1.64 (m, 3H), 1.52 (m, 14H) ppm. ESI-MS m/z calc. 644.24335, found 645.4 (M+1)⁺; Retention time: 2.09 minutes (LC Method M).

### Step 2: tert-Butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 1)

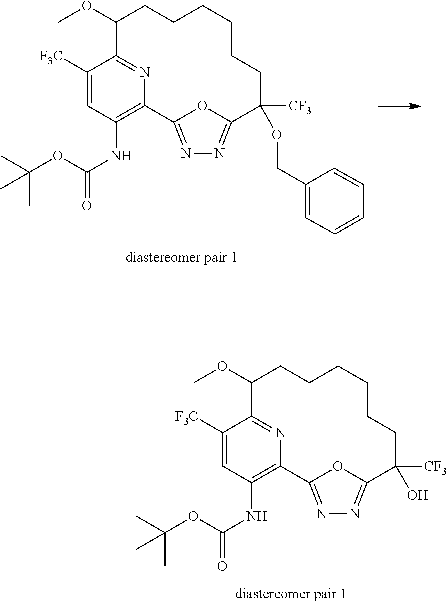 [SMILES: COC1CCCCCCC(OCc2ccccc2)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2NC(=O)OC(C)(C)C]

To a stirred solution of tert-butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (27 mg, 0.04189 mmol) in acetic acid (4 mL) in a Parr reactor was added Pd/C (11.8 mg, 10% w/w, 50% wet, 0.005544 mmol) under nitrogen gas. The Parr reactor was set up and filled with nitrogen gas and evacuated 3 times. Finally, the reactor was evacuated and filled with hydrogen gas up to 100 psi. The reaction was stirred at room temperature for 15 h. The reactor was evacuated and filled by nitrogen gas and the mixture was filtered through Celite pad and washed with ethyl acetate (10 mL). The filtrate was evaporated and the residue was purified by reverse phase chromatography eluting with a gradient of 50% acetonitrile in water to 100% acetonitrile giving tert-butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (17 mg, 73%). ESI-MS m/z calc. 554.1964, found 555.0 (M+1)⁺; Retention time: 1.93 minutes (LC Method J).

### Step 3: 17-Amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 1) (Compound 12) and 17-amino-13-methoxy-6,15-bis(trifluoromethyl)-19-oxa-3,4,18-triazatricyclo[12.3.1.12,5]nonadeca-1(18),2,4,14,16-pentaen-6-ol (Diastereomer pair 1, Enantiomer 2) (Compound 13)

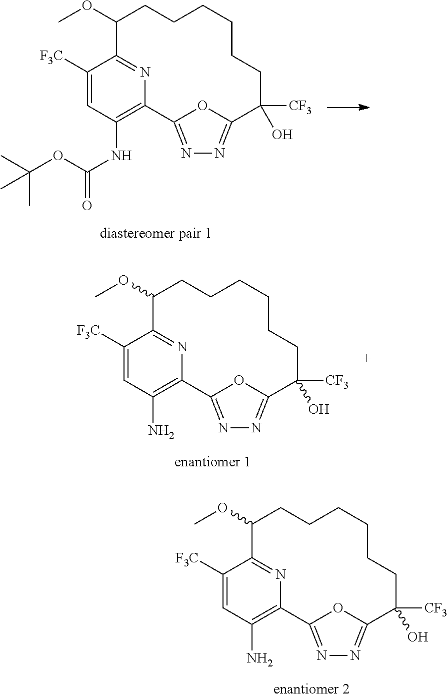 [SMILES: COC1CCCCCCC(O)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2N ; COC1CCCCCCC(O)(C(F)(F)F)c2nnc(o2)-c2nc1c(C(F)(F)F)cc2N]

To a stirred solution of tert-butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 1) (17 mg, 0.03066 mmol) in dichloromethane (2 mL) was added TFA (1 mL, 12.98 mmol) and the mixture was stirred for 25 minutes. After that time, the mixture was cooled in ice bath and 2 mL of saturated aqueous sodium bicarbonate was slowly added. The product was extracted with dichloromethane (2×5 mL). The organic layers were combined, dried over sodium sulfate, filtered and concentrated then purified by reverse phase chromatography eluting with a gradient of 50% acetonitrile in water to 100% acetonitrile over 7 minutes then eluting at 100% acetonitrile for 5 minutes giving a mixture of diastereomers. This material was further purified by chiral SFC chromatography using a ChiralPak AS column (250×10 mm, 5 m particle size) with 8% methanol (20 mM NH₃) and 92% carbon dioxide mobile phase at 10 mL/min (injection volume=70 μL of ˜24 mg/mL solution in 90:10 methanol/DMSO) which gave as a white solid and the first enantiomer to elute, 17-amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 1) (4.9 mg, 69%). ¹H NMR (400 MHz, DMSO-d₆) δ 7.74 (s, 1H), 7.58 (s, 1H), 6.91 (s, 2H), 4.50 (d, J=8.6 Hz, 1H), 3.29 (s, 3H), 2.16 (m, 3H), 1.83 (dd, J=14.1, 10.2 Hz, 1H), 1.64-1.37 (m, 8H) ppm. ESI-MS m/z calc. 454.14395, found 455.0 (M+1)⁺; Retention time: 1.28 minutes (LC Method J).

The later eluting enantiomer provided 17-amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 2) (4.7 mg, 66%). ¹H NMR (400 MHz, DMSO-d₆) δ 7.74 (s, 1H), 7.58 (s, 1H), 6.91 (s, 2H), 4.50 (d, J=8.6 Hz, 1H), 3.29 (s, 3H), 2.16 (m, 3H), 1.83 (dd, J=14.1, 10.2 Hz, 1H), 1.64-1.37 (m, 8H) ppm. ESI-MS m/z calc. 454.14395, found 455.0 (M+1)⁺; Retention time: 1.28 minutes (LC Method J).

### Example 7: Preparation of 17-Amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 3) (Compound 14) and 17-amino-13-methoxy-6,15-bis(trifluoromethyl)-19-oxa-3,4,18-triazatricyclo[12.3.1.12,5]nonadeca-1(18),2,4,14,16-pentaen-6-ol (Diastereomer Pair 2, Enantiomer 4) (Compound 15)

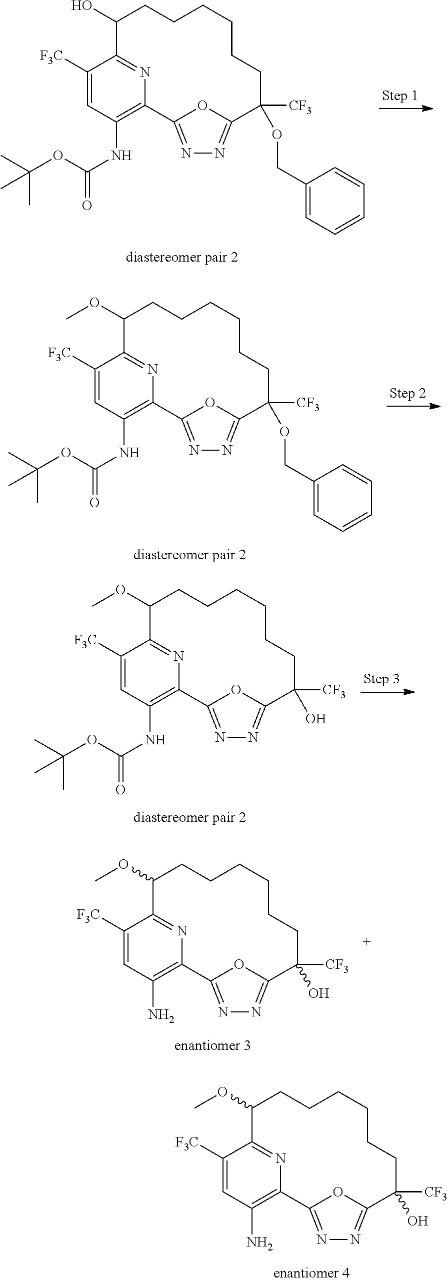 [SMILES: CC(C)(C)OC(=O)Nc1cc(C(F)(F)F)c2nc1-c1nnc(o1)C(OCc1ccccc1)(C(F)(F)F)CCCCCCC2O ; COC1CCCCCCC(OCc2ccccc2)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2N ; COC1CCCCCCC(O)(C(F)(F)F)c2nnc(o2)-c2nc1c(C(F)(F)F)cc2N]

### Step 1: tert-Butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 2)

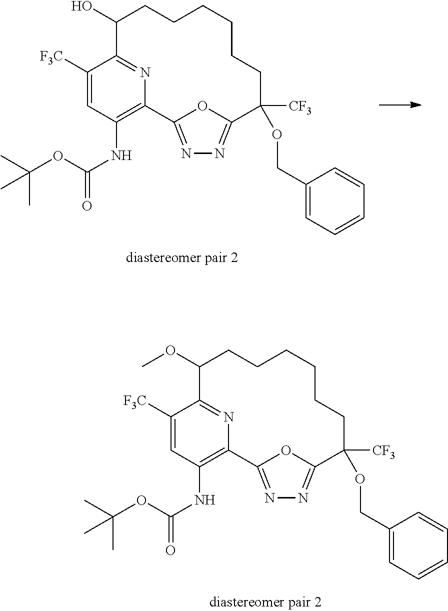 [SMILES: CC(C)(C)OC(=O)Nc1cc(C(F)(F)F)c2nc1-c1nnc(o1)C(OCc1ccccc1)(C(F)(F)F)CCCCCCC2O ; COC1CCCCCCC(OCc2ccccc2)(C(F)(F)F)c2nnc(o2)-c2nc1c(C(F)(F)F)cc2NC(=O)OC(C)(C)C]

To a stirred solution of tert-butyl N-[6-benzyloxy-13-hydroxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (60 mg, 0.09515 mmol) in THF (4 mL) at 0° C. (ice bath) was added potassium tert-butoxide (51.3 mg of 25% w/w, 0.1143 mmol) (2 M solution in 2-methyltetrahydrofuran) under an inert atmosphere. The mixture was stirred for 5 min and then iodomethane (16.2 mg, 0.1141 mmol) in THF (1 mL) was added slowly by a syringe via a rubber septum. The mixture was stirred for additional 30 minutes at room temperature. Next, the reaction mixture was cooled down and quenched at 0° C. by adding 10 mL water and the mixture was then extracted with diethyl ether (3×20 mL). The organic layers were combined, dried over sodium sulfate, filtered and concentrated then purified by silica gel chromatography eluting with 85:15 hexane/ethyl acetate which gave tert-butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (34 mg, 55%). ESI-MS m/z calc. 644.24335, found 645.0 (M+1)⁺; Retention time: 2.03 minutes (LC Method M).

### Step 2: tert-Butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (Diastereomer Pair 2)

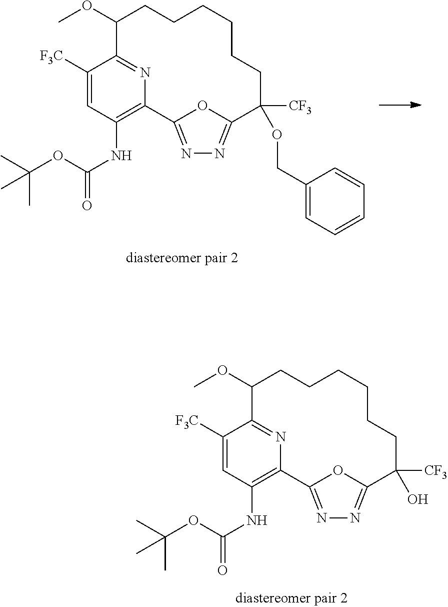 [SMILES: COC1CCCCCCC(OCc2ccccc2)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2NC(=O)OC(C)(C)C]

To a stirred solution of tert-butyl N-[6-benzyloxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (28 mg, 0.04344 mmol) in acetic acid (2 mL) in a high pressure Parr reactor was added Pd/C (8.5 mg, 10% w/w, 50% wet, 0.003947 mmol) under nitrogen atmosphere. The reactor evacuated and filled with nitrogen gas three times and finally filled with hydrogen gas to a pressure of 100 psi and stirred at room temperature for 16 h. The Parr reactor was then depressurized and filled with nitrogen gas. The mixture was filtered through a pad of Celite eluting with ethyl acetate. The filtrate was evaporated and the resulting residue was purified by reverse phase chromatography eluting with a gradient of 50% acetonitrile in water to 100% acetonitrile giving tert-butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (20 mg, 83%). ESI-MS m/z calc. 554.1964, found 555.0 (M+1)⁺; Retention time: 2.04 minutes (LC Method J).

### Step 3: 17-Amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 3) (Compound 14) and 17-amino-13-methoxy-6,15-bis(trifluoromethyl)-19-oxa-3,4,18-triazatricyclo[12.3.1.12,5]nonadeca-1(18),2,4,14,16-pentaen-6-ol (Diastereomer Pair 2, Enantiomer 4) (Compound 15)

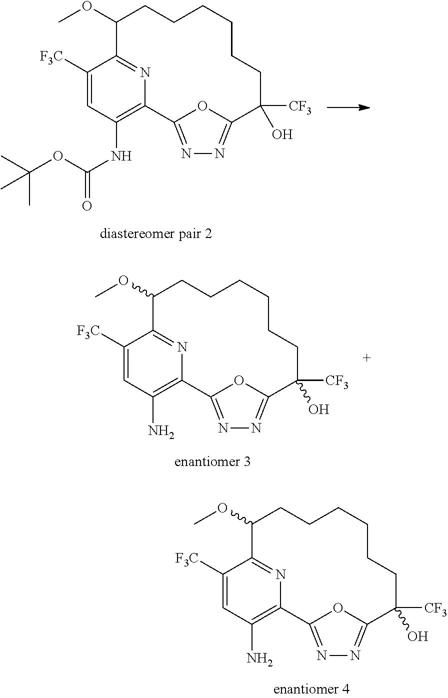 [SMILES: COC1CCCCCCC(O)(C(F)(F)F)c2nnc(o2)-c2nc1c(C(F)(F)F)cc2NC(=O)OC(C)(C)C ; COC1CCCCCCC(O)(C(F)(F)F)c2nnc(o2)-c2nc1c(C(F)(F)F)cc2N ; COC1CCCCCCC(O)(C(F)(F)F)c2nnc(o2)-c2nc1c(C(F)(F)F)cc2N]

To a stirred solution of tert-butyl N-[6-hydroxy-13-methoxy-6,15-bis(trifluoromethyl)-19-oxa-3,4,18-triazatricyclo[12.3.1.12,5]nonadeca-1(17),2,4,14(18),15-pentaen-17-yl]carbamate (diastereomer pair 2) (14 mg, 0.02525 mmol) in dichloromethane (2 mL) was added TFA (1 mL, 12.98 mmol) and the mixture was stirred for 25 minutes at room temperature. After that time, the mixture was cooled in ice bath and 2 mL of a saturated aqueous solution of sodium bicarbonate was slowly added. The product was extracted with dichloromethane (2×5 mL). The organic layers were combined, dried over sodium sulfate, filtered and concentrated then purified by reverse phase chromatography eluting with a gradient of 50% acetonitrile in water to 100% acetonitrile over 7 minutes then eluting at 100% acetonitrile for 5 minutes giving a mixture of diastereomers. This material was further purified by SFC chromatography using a ChiralPak IG column (250×10 mm, 5 m particle size) with 22% methanol (20 mM NH₃) and 78% carbon dioxide mobile phase at 10 mL/min (injection volume=70 μL of ˜20 mg/mL solution in methanol) which gave as a white solid and the first enantiomer to elute 17-amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 3) (2 mg, 34%). ¹H NMR (500 MHz, DMSO-d₆) δ 7.74 (s, 1H), 7.65 (d, J=12.7 Hz, 1H), 6.91 (s, 2H), 4.54 (d, J=7.7 Hz, 1H), 3.30 (s, 3H), 2.30-2.02 (m, 3H), 1.85-1.70 (m, 2H), 1.66-1.32 (m, 7H) ppm. ESI-MS m/z calc. 454.14395, found 455.0 (M+1)⁺; Retention time: 1.39 minutes (LC Method J).

The later eluting enantiomer provided 17-amino-13-methoxy-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 4) (2 mg, 34%). ¹H NMR (500 MHz, DMSO-d₆) δ 7.74 (s, 1H), 7.65 (d, J=12.7 Hz, 1H), 6.91 (s, 2H), 4.54 (d, J=7.7 Hz, 1H), 3.30 (s, 3H), 2.30-2.02 (m, 3H), 1.85-1.70 (m, 2H), 1.66-1.32 (m, 7H) ppm. ESI-MS m/z calc. 454.14395, found 455.0 (M+1)⁺; Retention time: 1.39 minutes (LC Method J).

### Example 8: Preparation of 17-Amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 1) (Compound 16), 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 2) (Compound 17), 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 3) (Compound 18), and 17-amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 2, Enantiomer 4) (Compound 19)

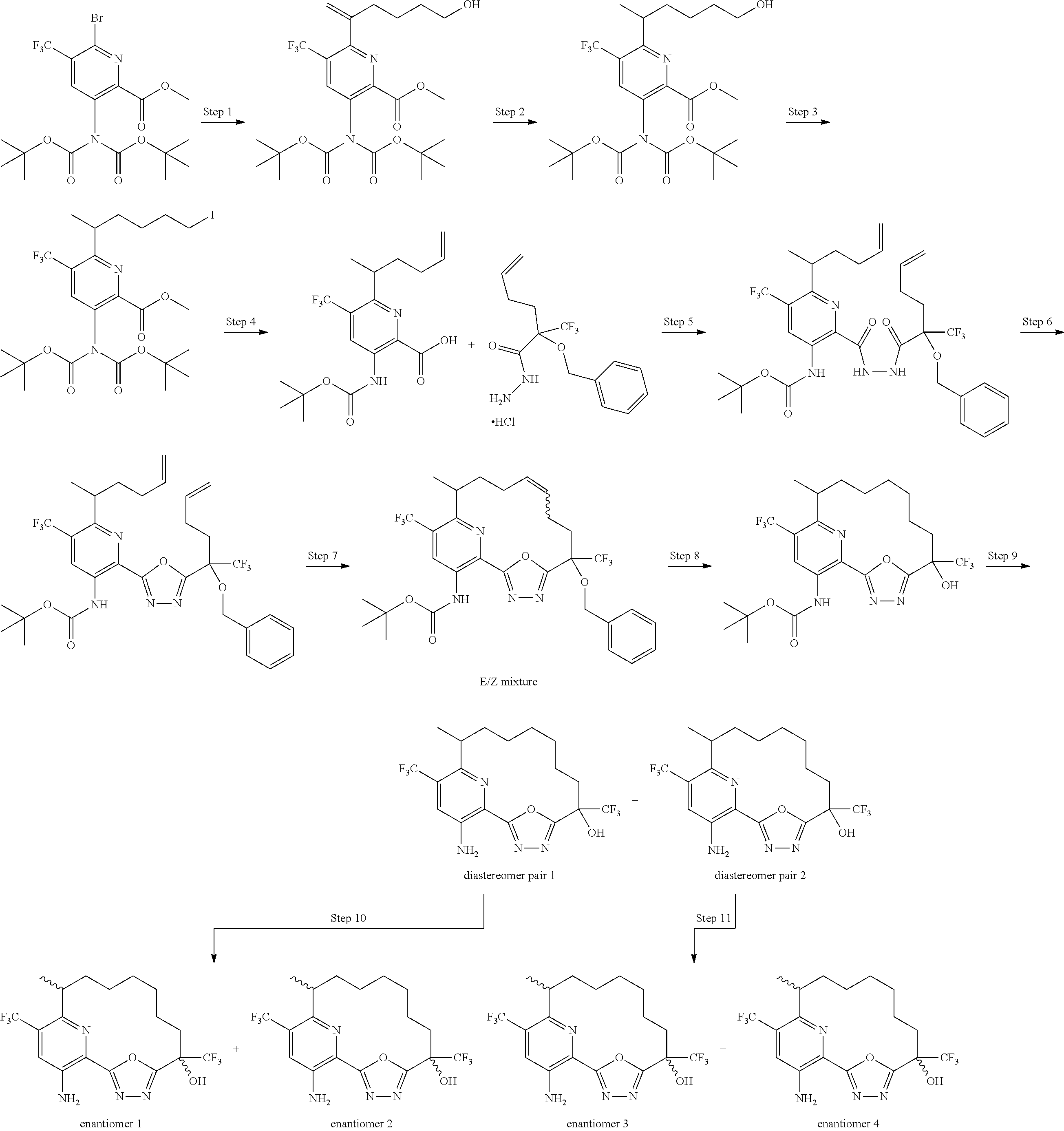

### Step 1: Methyl 3-[bis(tert-butoxycarbonyl)amino]-6-(5-hydroxy-1-methylene-pentyl)-5-(trifluoromethyl)pyridine-2-carboxylate

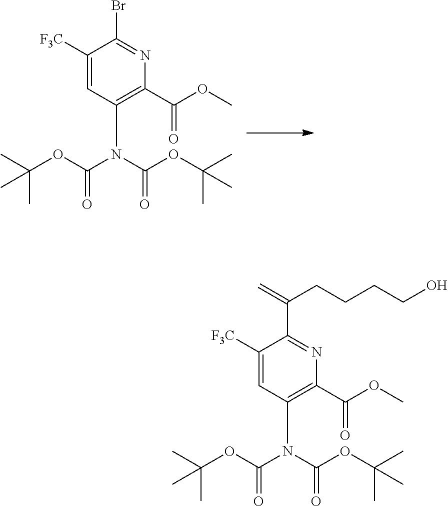 [SMILES: COC(=O)c1nc(Br)c(C(F)(F)F)cc1N(C(=O)OC(C)(C)C)C(=O)OC(C)(C)C ; C=C(CCCCO)c1nc(C(=O)OC)c(N(C(=O)OC(C)(C)C)C(=O)OC(C)(C)C)cc1C(F)(F)F]

To an oven dried flask (250 mL) equipped with magnetic stir bar was added methyl 3-[bis(tert-butoxycarbonyl)amino]-6-bromo-5-(trifluoromethyl)pyridine-2-carboxylate (1.9 g, 3.806 mmol), 5-tributylstannylhex-5-en-1-ol (1.64 g, 4.214 mmol), PdCl₂(PPh₃)₂ (267 mg, 0.3804 mmol), CuI (108.8 mg, 0.5713 mmol), and CsF (144.5 mg, 0.9513 mmol). The flask was evacuated and filled with nitrogen gas 3 times. Next, DMF (100 mL) was injected through a septum under an inert atmosphere. The mixture was heated at 80° C. for 7 h. The mixture was cooled to room temperature and filtered through Celite pad and washed with ethyl acetate. Most organic solvents were evaporated and the residue was purified by silica gel chromatography using 85:15 of hexanes and ethyl acetate providing methyl 3-[bis(tert-butoxycarbonyl)amino]-6-(5-hydroxy-1-methylene-pentyl)-5-(trifluoromethyl)pyridine-2-carboxylate (1.3 g, 66%). ¹H NMR (400 MHz, CDCl₃) δ 7.88 (s, 1H), 5.42 (d, J=1.6 Hz, 1H), 5.17 (s, 1H), 3.95 (s, 3H), 3.66 (t, J=6.2 Hz, 2H), 2.55 (t, J=7.4 Hz, 2H), 1.74-1.62 (m, 3H), 1.62-1.49 (m, 2H), 1.43 (s, 18H) ppm. ESI-MS m/z calc. 518.224, found 519.3 (M+1)⁺; Retention time: 1.34 minutes (LC Method J).

### Step 2: Methyl 3-[bis(tert-butoxycarbonyl)amino]-6-(5-hydroxy-1-methyl-pentyl)-5-(trifluoromethyl)pyridine-2-carboxylate

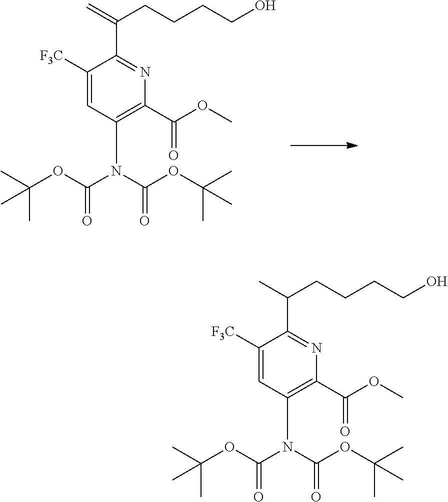 [SMILES: C=C(CCCCO)c1nc(C(=O)OC)c(N(C(=O)OC(C)(C)C)C(=O)OC(C)(C)C)cc1C(F)(F)F ; COC(=O)c1nc(C(C)CCCCO)c(C(F)(F)F)cc1N(C(=O)OC(C)(C)C)C(=O)OC(C)(C)C]

To a stirred solution of methyl 3-[bis(tert-butoxycarbonyl)amino]-6-(5-hydroxy-1-methylene-pentyl)-5-(trifluoromethyl)pyridine-2-carboxylate (400 mg, 0.7714 mmol) in MeOH (5 mL) was added Pd/C (49 mg, 10% w/w, 0.02321 mmol, 50% wet). The flask was evacuated and filled with nitrogen gas and finally it was filled with hydrogen gas by using two hydrogen balloons through a rubber septum. The reaction mixture was stirred for 2 hours. The mixture was filtered through a pad of Celite and washed with MeOH (10 mL). The solvent was evaporated and purified by silica gel chromatography using 80:20 of hexanes and ethyl acetate giving methyl 3-[bis(tert-butoxycarbonyl)amino]-6-(5-hydroxy-1-methyl-pentyl)-5-(trifluoromethyl)pyridine-2-carboxylate (350 mg, 87%). ¹H NMR (400 MHz, CDCl₃) δ 7.75 (s, 1H), 3.94 (s, 3H), 3.58 (m, 2H), 3.28 (q, J=7.0 Hz, 1H), 2.42-1.93 (m, 1H), 1.69-1.61 (m, 1H), 1.57-1.49 (m, 2H), 1.45 (s, 1H), 1.41 (s, 18H), 1.37-1.32 (m, 1H), 1.29 (d, J=6.7 Hz, 3H), 1.16-0.99 (m, 1H) ppm. ESI-MS m/z calc. 520.2396, found 521.1 (M+1)⁺; Retention time: 1.41 minutes (LC Method J).

### Step 3: Methyl 3-[bis(tert-butoxycarbonyl)amino]-6-(5-iodo-1-methyl-pentyl)-5-(trifluoromethyl)pyridine-2-carboxylate

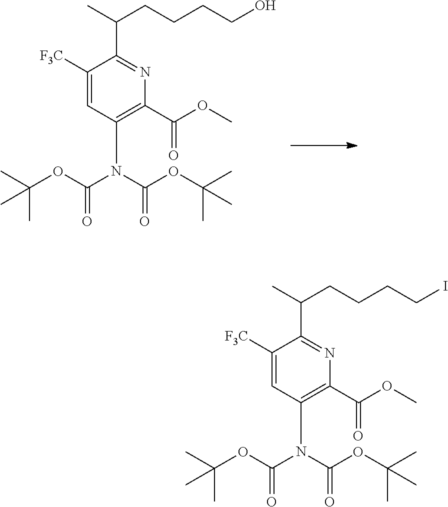 [SMILES: COC(=O)c1nc(C(C)CCCCO)c(C(F)(F)F)cc1N(C(=O)OC(C)(C)C)C(=O)OC(C)(C)C ; COC(=O)c1nc(C(C)CCCCI)c(C(F)(F)F)cc1N(C(=O)OC(C)(C)C)C(=O)OC(C)(C)C]

Methyl 3-[bis(tert-butoxycarbonyl)amino]-6-(5-hydroxy-1-methyl-pentyl)-5-(trifluoromethyl)pyridine-2-carboxylate (380 mg, 0.73 mmol), imidazole (99.4 mg, 1.46 mmol) and PPh₃ (210.7 mg, 0.8033 mmol) were dissolved in 2-methyltetrahydrofuran (10 mL) under nitrogen gas. Molecular iodine (203.9 mg, 0.8034 mmol) was dissolved in 0.5 mL of 2-methyltetrahydrofuran and added dropwise during 15 min. The reaction was stirred for 90 minutes at room temperature. The reaction mixture was filtered and washed with ethyl acetate and the solvent was evaporated. The residue was purified by silica gel chromatography using 85:15 of hexanes and ethyl acetate providing methyl 3-[bis(tert-butoxycarbonyl)amino]-6-(5-iodo-1-methyl-pentyl)-5-(trifluoromethyl)pyridine-2-carboxylate (345 mg, 75%). ¹H NMR (400 MHz, Chloroform-d) δ 7.76 (s, 1H), 3.94 (s, 3H), 3.27 (m, 1H), 3.12 (m, 2H), 1.98 (m, 1H), 1.78 (m, 2H), 1.65 (m, 1H), 1.41 (s, 18H), 1.35 (m, 1H), 1.30 (d, J=6.6 Hz, 3H), 1.17-1.05 (m, 1H) ppm. ESI-MS m/z calc. 630.14136, found 631.0 (M+1)⁺; Retention time: 1.52 minutes (LC Method J).

### Step 4: 3-(tert-Butoxycarbonylamino)-6-(1-methylpent-4-enyl)-5-(trifluoromethyl)pyridine-2-carboxylic Acid

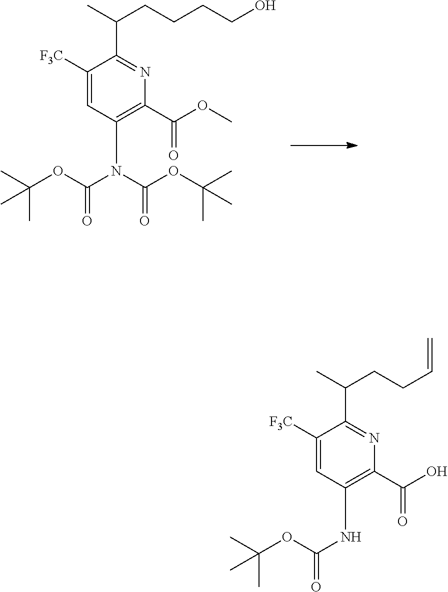 [SMILES: COC(=O)c1nc(C(C)CCCCO)c(C(F)(F)F)cc1N(C(=O)OC(C)(C)C)C(=O)OC(C)(C)C ; C=CCCC(C)c1nc(C(=O)O)c(NC(=O)OC(C)(C)C)cc1C(F)(F)F]

To a stirred solution of methyl 3-[bis(tert-butoxycarbonyl)amino]-6-(5-iodo-1-methyl-pentyl)-5-(trifluoromethyl)pyridine-2-carboxylate (400 mg, 0.6345 mmol) in THF (20 mL) under inert atmosphere, sodium tert-butoxide (305 mg, 3.174 mmol) was added at 0° C. The reaction mixture was allowed to reach to room temperature and stirred for 3 hours and then, heated at 50° C. for 1 h. The mixture was cooled to room temperature and aqueous HCl solution (5% of 15 mL) was added and the organic compounds was extracted with ethyl acetate (3×10 mL). The organic layers were combined, dried over sodium sulfate, concentrated and the residue was purified by silica gel chromatography using 85:15 of hexanes and ethyl acetate giving 3-(tert-butoxycarbonylamino)-6-(1-methylpent-4-enyl)-5-(trifluoromethyl)pyridine-2-carboxylic acid (150 mg, 61%). ¹H NMR (400 MHz, CDCl₃) δ 10.11 (s, 1H), 9.25 (s, 1H), 5.72 (m, 1H), 5.13-4.76 (m, 2H), 3.28 (q, J=6.7 Hz, 1H), 2.25-1.99 (m, 1H), 1.89 (tdd, J=14.7, 7.8, 4.5 Hz, 2H), 1.78-1.68 (m, 1H), 1.54 (s, 9H), 1.28 (d, J=6.7 Hz, 4H) ppm. ESI-MS m/z calc. 388.16098, found 389.0 (M+1)⁺; Retention time: 1.25 minutes (LC Method M).

### Step 5: tert-Butyl N-[2-[[[2-benzyloxy-2-(trifluoromethyl)hex-5-enoyl]amino]carbamoyl]-6-(1-methylpent-4-enyl)-5-(trifluoromethyl)-3-pyridyl]carbamate

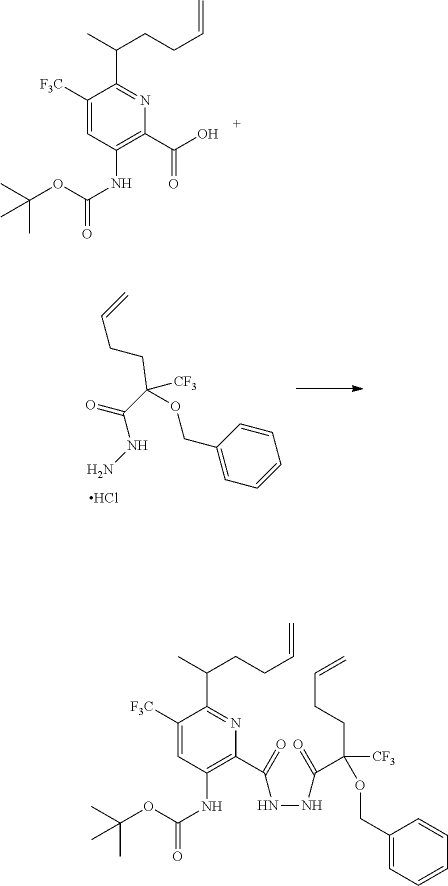 [SMILES: C=CCCC(C)c1nc(C(=O)O)c(NC(=O)OC(C)(C)C)cc1C(F)(F)F ; C=CCCC(OCc1ccccc1)(C(=O)NN)C(F)(F)F ; Cl ; C=CCCC(C)c1nc(C(=O)NNC(=O)C(CCC=C)(OCc2ccccc2)C(F)(F)F)c(NC(=O)OC(C)(C)C)cc1C(F)(F)F]

To a solution of 3-(tert-butoxycarbonylamino)-6-(1-methylpent-4-enyl)-5-(trifluoromethyl)pyridine-2-carboxylic acid (230 mg, 0.5922 mmol) and 2-benzyloxy-2-(trifluoromethyl)hex-5-enehydrazide (hydrochloride salt) (197 mg, 0.5815 mmol) in NMP (7 mL) was added DIEA (115 mg, 0.8898 mmol), followed by HATU (338 mg, 0.8889 mmol). The reaction mixture was stirred at room temperature for 1 h. The mixture was diluted with water (100 mL) and extracted with ethyl acetate (3×50 mL). The organic phases combined and dried over Na₂SO₄, filtered, and concentrated in vacuo. Purification by silica gel chromatography using a gradient from 0% to 15% ethyl acetate in hexanes provided tert-butyl N-[2-[[[2-benzyloxy-2-(trifluoromethyl)hex-5-enoyl]amino]carbamoyl]-6-(1-methylpent-4-enyl)-5-(trifluoromethyl)-3-pyridyl]carbamate (280 mg, 70%). ¹H NMR (400 MHz, CDCl₃) δ 10.51 (s, 1H), 10.11 (dd, J=13.8, 5.6 Hz, 1H), 9.13 (d, J=7.5 Hz, 2H), 7.61-7.22 (m, 5H), 5.98-5.81 (m, 1H), 5.80-5.62 (m, 1H), 5.21-5.00 (m, 2H), 5.01-4.79 (m, 3H), 4.74 (d, J=10.6 Hz, 1H), 3.28-3.20 (m, 1H), 2.51-2.44 (m, 1H), 2.32-2.21 (m, 3H), 2.10-1.97 (m, 1H), 1.95-1.84 (m, 2H), 1.75-1.68 (m, 1H), 1.53 (s, 9H), 1.26 (dd, J=6.7, 3.6 Hz, 3H) ppm. ESI-MS m/z calc. 672.27466, found 673.3 (M+1)⁺; Retention time: 1.9 minutes (LC Method M).

### Step 6: tert-Butyl N-[2-[5-[1-benzyloxy-1-(trifluoromethyl)pent-4-enyl]-1,3,4-oxadiazol-2-yl]-6-(1-methylpent-4-enyl)-5-(trifluoromethyl)-3-pyridyl]carbamate

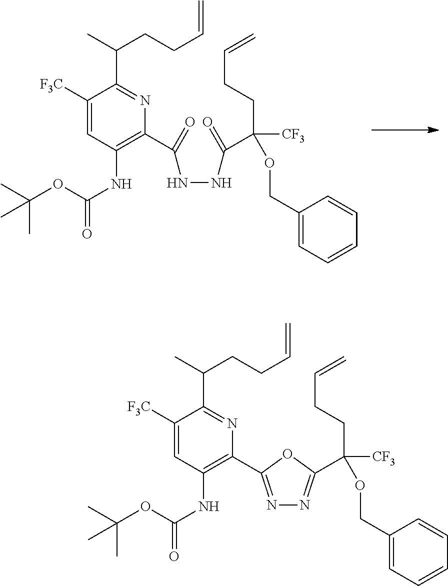 [SMILES: C=CCCC(C)c1nc(C(=O)NNC(=O)C(CCC=C)(OCc2ccccc2)C(F)(F)F)c(NC(=O)OC(C)(C)C)cc1C(F)(F)F ; C=CCCC(C)c1nc(-c2nnc(C(CCC=C)(OCc3ccccc3)C(F)(F)F)o2)c(NC(=O)OC(C)(C)C)cc1C(F)(F)F]

A solution of tert-butyl N-[2-[[[2-benzyloxy-2-(trifluoromethyl)hex-5-enoyl]amino]carbamoyl]-6-(1-methylpent-4-enyl)-5-(trifluoromethyl)-3-pyridyl]carbamate (280 mg, 0.4163 mmol) and DIEA (134.5 mg, 1.041 mmol) in CH₃CN (20 mL) was heated to 50° C. Then, p-TsCl (95.3 mg, 0.4999 mmol) was added and the mixture was heated at 70° C. for 90 minutes. The reaction mixture was cooled, quenched with saturated solution of sodium bicarbonate (10 mL) and extracted with ethyl acetate (3×30 mL). Organic layers combined and washed with brine (20 mL), dried over sodium sulfate, filtered and solvent evaporated. Purification by silica gel chromatography using a gradient from 0% to 5% ethyl acetate in hexanes provided tert-butyl N-[2-[5-[1-benzyloxy-1-(trifluoromethyl)pent-4-enyl]-1,3,4-oxadiazol-2-yl]-6-(1-methylpent-4-enyl)-5-(trifluoromethyl)-3-pyridyl]carbamate (225 mg, 83%). ¹H NMR (400 MHz, CDCl₃) δ 10.05 (d, J=2.7 Hz, 1H), 9.17 (s, 1H), 7.58-7.41 (m, 2H), 7.39-7.20 (m, 3H), 5.89-5.58 (m, 2H), 5.05 (m, 1H), 5.00-4.78 (m, 4H), 4.71 (dd, J=10.7, 7.1 Hz, 1H), 3.46-3.20 (m, 1H), 2.61-2.35 (m, 3H), 2.33-2.19 (m, 1H), 2.11-2.00 (m, 2H), 1.85 (m, 1H), 1.76-1.65 (m, 1H), 1.56 (s, 9H), 1.28 (dd, J=6.6, 3.0 Hz, 3H) ppm. ESI-MS m/z calc. 654.2641, found 655.0 (M+1)⁺; Retention time: 1.19 minutes (LC Method M).

### Step 7: tert-Butyl N-[6-benzyloxy-13-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture)

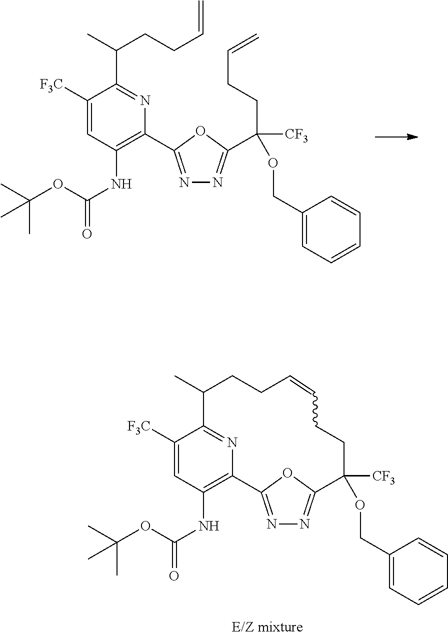 [SMILES: C=CCCC(C)c1nc(-c2nnc(C(CCC=C)(OCc3ccccc3)C(F)(F)F)o2)c(NC(=O)OC(C)(C)C)cc1C(F)(F)F ; CC1CCC=CCCC(OCc2ccccc2)(C(F)(F)F)C2=NN=C(O2)c2nc1c(C(F)(F)F)cc2NC(=O)OC(C)(C)C]

In a three necked 500 mL flask, [1,3-bis-(2-tolyl)-2-imidazolidinylidene]dichloro(2-isopropoxybenzylidene)ruthenium(II) (19.2 mg, 0.03365 mmol) was dissolved in 1,2-dichloroethane (70 mL) and bubbled nitrogen gas in the solution through a long needle and the flask was heated to 60° C. A solution of tert-butyl N-[2-[5-[1-benzyloxy-1-(trifluoromethyl)pent-4-enyl]-1,3,4-oxadiazol-2-yl]-6-(1-methylpent-4-enyl)-5-(trifluoromethyl)-3-pyridyl]carbamate (220 mg, 0.3361 mmol) in 1,2-dichloroethane (70 mL) was added slowly dropwise during 45 min using a dropping funnel at 60° C. The reaction mixture was stirred at 75° C. for 7 hours. The reaction mixture was cooled to room temperature and solvent was evaporated. Purification by silica gel chromatography using 95:5 of hexanes and ethyl acetate provided tert-butyl N-[6-benzyloxy-13-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z mixture) (60 mg, 28%). ESI-MS m/z calc. 626.2328, found 627.0 (M+1)⁺; Retention time: 1.21 minutes and 1.25 minutes (LC Method M).

### Step 8: tert-Butyl N-[6-hydroxy-13-methyl-6,15-bis(trifluoromethyl)-19-oxa-3,4,18-triazatricyclo[12.3.1.12,5]nonadeca-1(18),2,4,14,16-pentaen-17-yl]carbamate

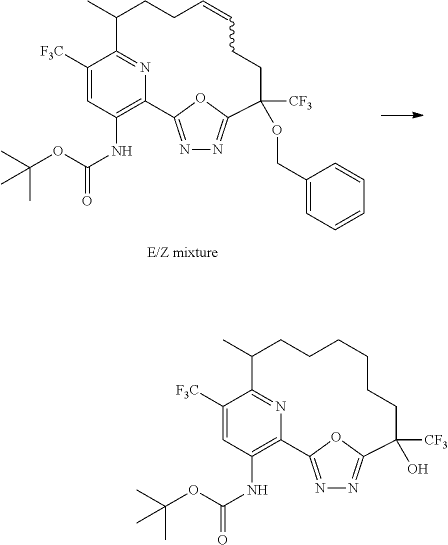 [SMILES: CC1CCC=CCCC(OCc2ccccc2)(C(F)(F)F)c2nnc(o2)-c2nc1c(C(F)(F)F)cc2NC(=O)OC(C)(C)C ; CC1CCCCCCC(O)(C(F)(F)F)c2nnc(o2)-c2nc1c(C(F)(F)F)cc2NC(=O)OC(C)(C)C]

To a stirred solution of tert-butyl N-[6-benzyloxy-13-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture) (58 mg, 0.09256 mmol) in acetic acid (3 mL) was added Pd/C (50 mg, 0.02349 mmol, 50% wet, 10% w/w) in a high pressure reactor and filled with nitrogen gas and evacuated three times. The reactor was filled with hydrogen gas up to 250 psi and stirred for 20 h. The mixture was filtered through a pad of Celite by washing with ethyl acetate and solvent evaporated. Purification by silica gel chromatography using 85:15 of hexanes and ethyl acetate provided tert-butyl N-[6-hydroxy-13-methyl-6,15-bis(trifluoromethyl)-19-oxa-3,4,18-triazatricyclo[12.3.1.12,5]nonadeca-1(18),2,4,14,16-pentaen-17-yl]carbamate (37 mg, 74%). ESI-MS m/z calc. 538.2015, found 539.1 (M+1)⁺; Retention time: 1.82 minutes and 1.86 minutes (LC Method M).

### Step 9: 17-Amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1) and 17-amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 2)

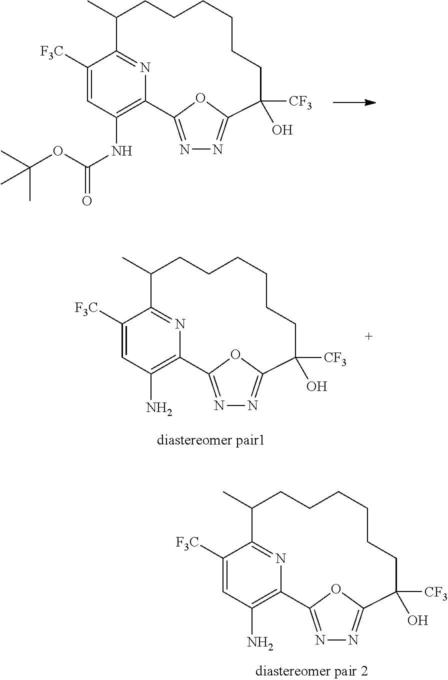 [SMILES: CC1CCCCCCC(O)(C(F)(F)F)c2nnc(o2)-c2nc1c(C(F)(F)F)cc2NC(=O)OC(C)(C)C ; CC1CCCCCCC(O)(C(F)(F)F)c2nnc(o2)-c2nc1c(C(F)(F)F)cc2N ; CC1CCCCCCC(O)(C(F)(F)F)c2nnc(o2)-c2nc1c(C(F)(F)F)cc2N]

To a stirred solution of tert-butyl N-[6-hydroxy-13-methyl-6,15-bis(trifluoromethyl)-19-oxa-3,4,18-triazatricyclo[12.3.1.12,5]nonadeca-1(18),2,4,14,16-pentaen-17-yl]carbamate (36 mg, 0.06685 mmol) in dichloromethane (2 mL) was added TFA (2 mL, 25.96 mmol) and the mixture was stirred for 25 minutes at room temperature. Solvents were evaporated and purification by silica gel chromatography using a gradient from 0% to 20% ethyl acetate in hexanes provided first eluting diastereomer pair, 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1) (13 mg, 89%). ESI-MS m/z calc. 438.14905, found 439.3 (M+1)⁺; Retention time: 1.71 minutes (LC Method J).

The later eluting diastereomer pair provided 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 2) (11 mg, 75%). ESI-MS m/z calc. 438.14905, found 439.1 (M+1)⁺; Retention time: 1.77 minutes (LC Method J).

### Step 10: 17-Amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 1, Enantiomer 1) (Compound 16) and 17-amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 1, Enantiomer 2) (Compound 17)

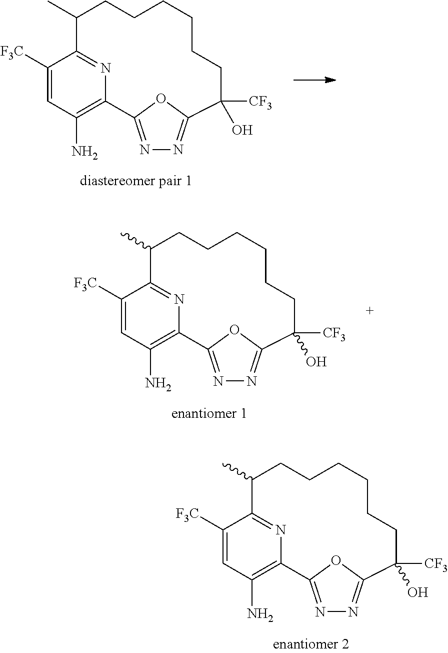

17-Amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1) (12 mg, 0.02737 mmol) was purified by chiral SFC using a ChiralPak IC column (250×10 mm, 5 μm particle size), 9% methanol (20 mM NH₃) and 91% carbon dioxide mobile phase with a flow rate of 10 mL/min over 5.0 min (injection volume 70 L of ˜21 mg/mL solution in methanol) which gave as a colorless solid and the first enantiomer to elute, 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 1) (3 mg, 49%). ESI-MS m/z calc. 438.14905, found 439.2 (M+1)⁺; Retention time: 1.71 minutes (LC Method J).

The second enantiomer to elute was isolated as a colorless solid, 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 1, enantiomer 2) (2.6 mg, 42%). ESI-MS m/z calc. 438.14905, found 439.2 (M+1)⁺; Retention time: 1.71 minutes (LC Method J).

### Step 11: 17-Amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 2, Enantiomer 3) (Compound 18) and 17-amino-13-methyl-6,15-bis(trifluoromethyl)-19-oxa-3,4,18-triazatricyclo[12.3.1.12,5]nonadeca-1(18),2,4,14,16-pentaen-6-ol (Diastereomer Pair 2, Enantiomer 4) (Compound 19)

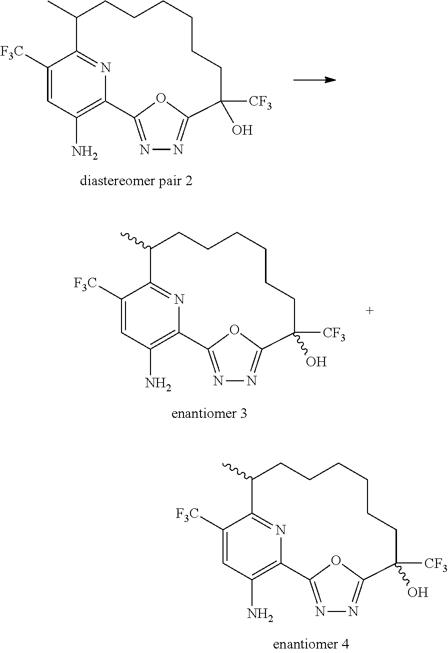

17-Amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 2) (11 mg, 0.02509 mmol) was purified by chiral SFC using a ChiralPak IC column (250×10 mm 5 μm particle size), 9% methanol (20 mM NH₃) and 91% carbon dioxide mobile phase at 10 mL/min over 5.0 min (injection volume 70 μL of ˜21 mg/mL solution in methanol) which gave as a colorless solid and the first enantiomer to elute 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 3) (3 mg, 53%). ESI-MS m/z calc. 438.14905, found 439.2 (M+1)⁺; Retention time: 1.77 minutes (LC Method J).

The second enantiomer to elute was isolated as a colorless solid, 17-amino-13-methyl-6,15-bis(trifluoromethyl)-19-oxa-3,4,18-triazatricyclo[12.3.1.12,5]nonadeca-1(18),2,4,14,16-pentaen-6-ol (diastereomer pair 2, enantiomer 4) (2.6 mg, 46%). ESI-MS m/z calc. 438.14905, found 439.1 (M+1)⁺; Retention time: 1.77 minutes (LC Method J).

### Example 9: Preparation of 1-[17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (enantiomer 1) (Compound 20) and 1-[17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (Enantiomer 2) (Compound 21)

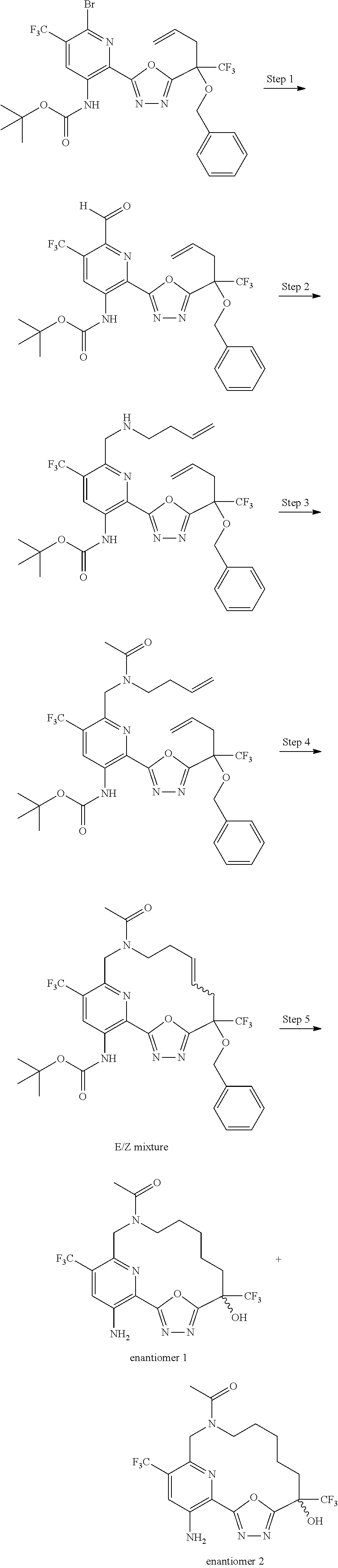

### Step 1: tert-Butyl N-[2-[5-[1-benzyloxy-1-(trifluoromethyl)but-3-enyl]-1,3,4-oxadiazol-2-yl]-6-formyl-5-(trifluoromethyl)-3-pyridyl]carbamate

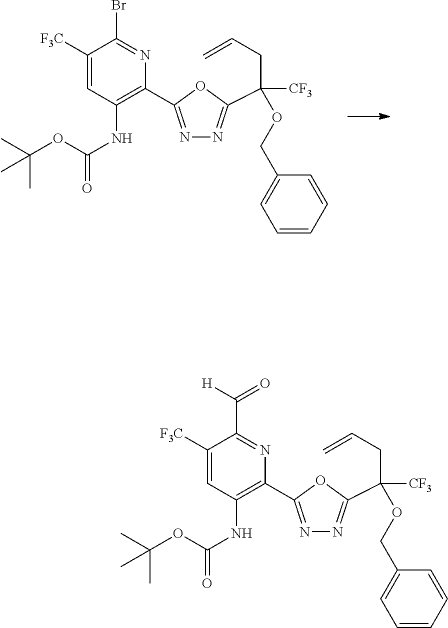 [SMILES: C=CCC(OCc1ccccc1)(c1nnc(-c2nc(Br)c(C(F)(F)F)cc2NC(=O)OC(C)(C)C)o1)C(F)(F)F ; C=CCC(OCc1ccccc1)(c1nnc(-c2nc(C=O)c(C(F)(F)F)cc2NC(=O)OC(C)(C)C)o1)C(F)(F)F]

To tert-butyl N-[2-[5-[1-benzyloxy-1-(trifluoromethyl)but-3-enyl]-1,3,4-oxadiazol-2-yl]-6-bromo-5-(trifluoromethyl)-3-pyridyl]carbamate (147 mg, 0.2191 mmol) in ether (2.2 mL) at −78° C. was added n-BuLi (210 μL of 2.5 M, 0.525 mmol) and the mixture was stirred at −78° C. for 15 min then pre-dried (molecular sieves) DMF (220 μL of 3 M, 0.66 mmol) as a solution in ether was added. The mixture was stirred at −78° C. for 15 min and then at 0° C. for 15 min, diluted with ether and 1 M NH₄Cl and partitioned. The organic layer was washed with brine, dried (MgSO₄), filtered and evaporated. The residue was purified by silica gel chromatography using a gradient from 0% to 10% ethyl acetate in hexanes which provided tert-butyl N-[2-[5-[1-benzyloxy-1-(trifluoromethyl)but-3-enyl]-1,3,4-oxadiazol-2-yl]-6-formyl-5-(trifluoromethyl)-3-pyridyl]carbamate (52 mg, 40%). ¹H NMR (400 MHz, Chloroform-d) δ 10.52 (s, 1H), 10.12 (t, J=1.0 Hz, 1H), 9.48 (s, 1H), 7.49-7.42 (m, 2H), 7.36 (m, 2H), 7.32-7.27 (m, 1H), 6.05-5.85 (m, 1H), 5.27 (m, 1H), 5.21 (dd, J=10.2, 1.4 Hz, 1H), 4.85 (d, J=10.6 Hz, 1H), 4.72 (d, J=10.7 Hz, 1H), 3.22 (m, 2H), 1.58 (s, 9H) ppm. ESI-MS m/z calc. 586.1651, found 587.2 (M+1)⁺; Retention time: 0.51 minutes (LC Method T).

### Step 2: tert-Butyl N-[2-[5-[1-benzyloxy-1-(trifluoromethyl)but-3-enyl]-1,3,4-oxadiazol-2-yl]-6-[(but-3-enylamino)methyl]-5-(trifluoromethyl)-3-pyridyl]carbamate

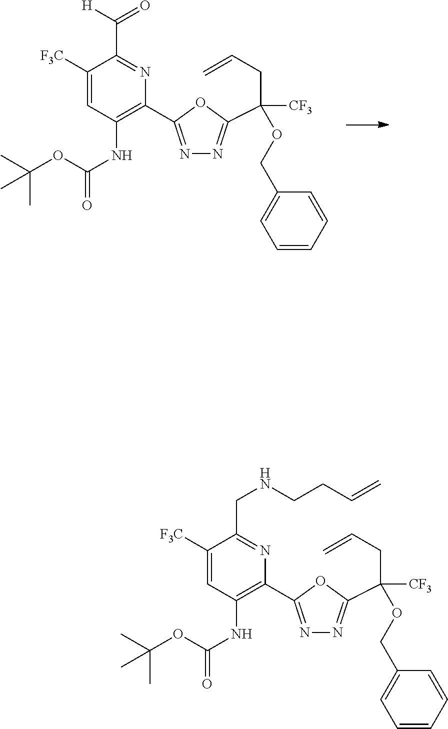 [SMILES: C=CCC(OCc1ccccc1)(c1nnc(-c2nc(C=O)c(C(F)(F)F)cc2NC(=O)OC(C)(C)C)o1)C(F)(F)F ; C=CCCNCc1nc(-c2nnc(C(CC=C)(OCc3ccccc3)C(F)(F)F)o2)c(NC(=O)OC(C)(C)C)cc1C(F)(F)F]

To a solution of tert-butyl N-[2-[5-[1-benzyloxy-1-(trifluoromethyl)but-3-enyl]-1,3,4-oxadiazol-2-yl]-6-formyl-5-(trifluoromethyl)-3-pyridyl]carbamate (59 mg, 0.1006 mmol) in dichloromethane (1.2 mL) was added acetic acid (100 μL of 2 M, 0.2 mmol) as a solution in THF followed by but-3-en-1-amine (14 mg, 0.1968 mmol). This mixture was stirred at room temperature for 15 min and then sodium triacetoxyborohydride (43 mg, 0.2029 mmol) was added. After stirring at room temperature for 30 min the mixture was diluted with ether, washed with 1 M NaHCO₃, dried (MgSO₄), filtered and evaporated to provide tert-butyl N-[2-[5-[1-benzyloxy-1-(trifluoromethyl)but-3-enyl]-1,3,4-oxadiazol-2-yl]-6-[(but-3-enylamino)methyl]-5-(trifluoromethyl)-3-pyridyl]carbamate (63 mg, 98%). ¹H NMR (400 MHz, Chloroform-d) δ 10.13 (s, 1H), 9.25 (s, 1H), 7.42-7.38 (m, 2H), 7.34 (m, 2H), 7.31-7.27 (m, 1H), 5.93 (m, 1H), 5.78 (m, 1H), 5.26 (dd, J=17.1, 1.5 Hz, 1H), 5.21 (dd, J=10.2, 1.4 Hz, 1H), 5.07 (m, 1H), 5.03-4.98 (m, 1H), 4.84 (d, J=10.8 Hz, 1H), 4.69 (d, J=10.8 Hz, 1H), 4.09 (d, J=1.2 Hz, 2H), 3.23 (q, J=7.4, 6.6 Hz, 2H), 2.75 (t, J=6.8 Hz, 2H), 2.28 (qt, J=6.9, 1.4 Hz, 2H), 1.72 (s, 1H), 1.56 (s, 9H) ppm. ESI-MS m/z calc. 641.24365, found 642.3 (M+1)⁺; Retention time: 0.67 minutes (LC Method T).

### Step 3: tert-Butyl N-[6-[[acetyl(but-3-enyl)amino]methyl]-2-[5-[1-benzyloxy-1-(trifluoromethyl)but-3-enyl]-1,3,4-oxadiazol-2-yl]-5-(trifluoromethyl)-3-pyridyl]carbamate

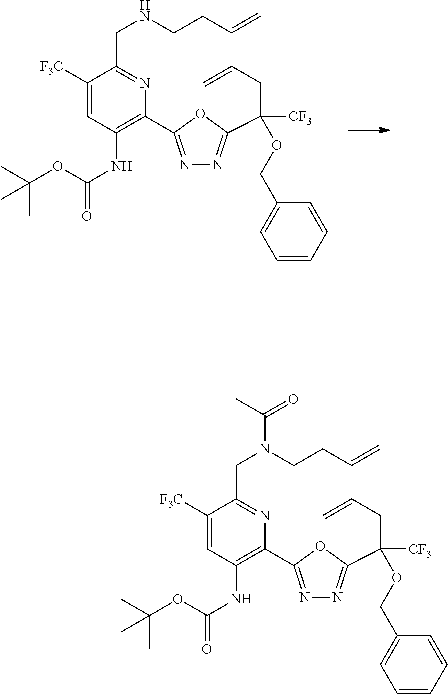 [SMILES: C=CCCNCc1nc(-c2nnc(C(CC=C)(OCc3ccccc3)C(F)(F)F)o2)c(NC(=O)OC(C)(C)C)cc1C(F)(F)F ; C=CCCN(Cc1nc(-c2nnc(C(CC=C)(OCc3ccccc3)C(F)(F)F)o2)c(NC(=O)OC(C)(C)C)cc1C(F)(F)F)C(C)=O]

DMF (500 μL) was added to tert-butyl N-[2-[5-[1-benzyloxy-1-(trifluoromethyl)but-3-enyl]-1,3,4-oxadiazol-2-yl]-6-[(but-3-enylamino)methyl]-5-(trifluoromethyl)-3-pyridyl]carbamate (34 mg, 0.05299 mmol) and HATU (40 mg, 0.1052 mmol) and then DIEA (37 μL, 0.2124 mmol) and acetic acid (53 μL of 2 M in THF, 0.106 mmol) were added. The mixture was stirred at room temperature for 1 h then diluted with ether. The resulting solution was washed with water then 1 M NaHCO₃, dried (MgSO₄), filtered and evaporated to provide tert-butyl N-[6-[[acetyl(but-3-enyl)amino]methyl]-2-[5-[1-benzyloxy-1-(trifluoromethyl)but-3-enyl]-1,3,4-oxadiazol-2-yl]-5-(trifluoromethyl)-3-pyridyl]carbamate (30.8 mg, 85%). ESI-MS m/z calc. 683.2542, found 684.3 (M+1)⁺; Retention time: 0.54 minutes (LC Method T).

### Step 4: tert-Butyl N-[12-acetyl-6-benzyloxy-6,15-bis(trifluoromethyl)-19-oxa-3,4,12,18-tetrazatricyclo[12.3.1.12,5]nonadeca-1(18),2,4,8,14,16-hexaen-17-yl]carbamate (E/Z Mixture)

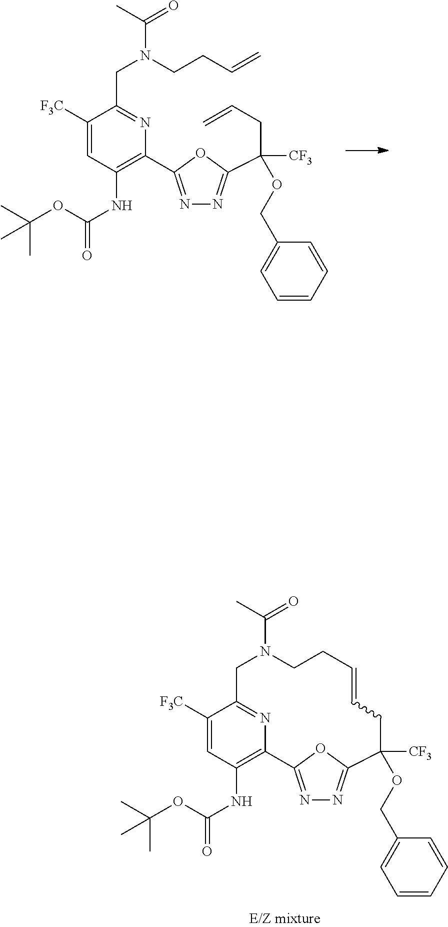 [SMILES: C=CCCN(Cc1nc(-c2nnc(C(CC=C)(OCc3ccccc3)C(F)(F)F)o2)c(NC(=O)OC(C)(C)C)cc1C(F)(F)F)C(C)=O ; CC(=O)N1CCC=CCC(OCc2ccccc2)(C(F)(F)F)c2nnc(o2)-c2nc(c(C(F)(F)F)cc2NC(=O)OC(C)(C)C)C1]

To a 25 mL three neck flask with benzylidene[1,3-bis(2,4,6-trimethylphenyl)-2-imidazolidinylidene]dichloro(tricyclohexylphosphine)ruthenium (5.8 mg, 0.0068 mmol) and toluene (2.35 mL) heated with a 120° C. heat bowl was added a solution of tert-butyl N-[6-[[acetyl(but-3-enyl)amino]methyl]-2-[5-[1-benzyloxy-1-(trifluoromethyl)but-3-enyl]-1,3,4-oxadiazol-2-yl]-5-(trifluoromethyl)-3-pyridyl]carbamate (31 mg, 0.045 mmol) in toluene (2.35 mL) and the mixture heated at 120° C. for 1 h. Then, a solution of benzylidene[1,3-bis(2,4,6-trimethylphenyl)-2-imidazolidinylidene]dichloro(tricyclohexylphosphine)ruthenium (4 mg, 0.0047 mmol) in toluene (0.5 mL) was added dropwise to the reaction mixture stirring in a 125° C. hot bowl and the mixture heated an additional 30 min at 120° C. The solvent was evaporated and the residue purified by silica gel chromatography using a gradient from 0% to 40% ethyl acetate in hexanes to provide tert-butyl N-[12-acetyl-6-benzyloxy-6,15-bis(trifluoromethyl)-19-oxa-3,4,12,18-tetrazatricyclo[12.3.1.12,5]nonadeca-1(18),2,4,8,14,16-hexaen-17-yl]carbamate (E/Z Mixture) (13 mg, 44%). ESI-MS m/z calc. 655.22296, found 656.3 (M+1)⁺; Retention time: 0.44 minutes (LC Method T).

### Step 5: 1-[17-Amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (enantiomer 1) (Compound 20) and 1-[17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (Enantiomer 2) (Compound 21)

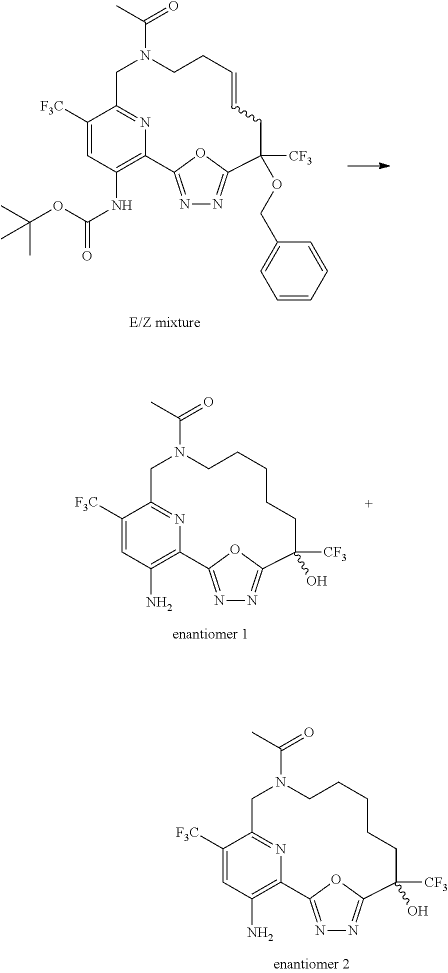 [SMILES: CC(=O)N1CCC=CCC(OCc2ccccc2)(C(F)(F)F)c2nnc(o2)-c2nc(c(C(F)(F)F)cc2NC(=O)OC(C)(C)C)C1 ; CC(=O)N1CCCCCC(O)(C(F)(F)F)c2nnc(o2)-c2nc(c(C(F)(F)F)cc2N)C1 ; CC(=O)N1CCCCCC(O)(C(F)(F)F)c2nnc(o2)-c2nc(c(C(F)(F)F)cc2N)C1]

Part 1: A mixture of tert-butyl N-[12-acetyl-6-benzyloxy-6,15-bis(trifluoromethyl)-19-oxa-3,4,12,18-tetrazatricyclo[12.3.1.12,5]nonadeca-1(18),2,4,8,14,16-hexaen-17-yl]carbamate (E/Z Mixture) (13 mg, 0.01983 mmol), 10% palladium on carbon (6 mg, 0.005638 mmol) and acetic acid (300 μL) was stirred at room temperature under 200 psi hydrogen gas in a stainless steel vessel for 21 h then the mixture was filtered. The filtrate was evaporated to provide the intermediate tert-butyl N-[12-acetyl-6-hydroxy-6,15-bis(trifluoromethyl)-19-oxa-3,4,12,18-tetrazatricyclo[12.3.1.12,5]nonadeca-1(18),2,4,14,16-pentaen-17-yl]carbamate (14.7 mg, quant.). ESI-MS m/z calc. 567.19165, found 568.2 (M+1)⁺; Retention time: 0.7 minutes (LC Method T).

Part 2: The residue from part 1 was dissolved in TFA (0.3 mL), triisopropylsilane (14 μL, 0.06834 mmol) and water (15 μL) and stirred for 1 h at room temperature. The solvent was evaporated and the residue was dissolved in ethyl acetate and washed with 1 M NaHCO₃, dried (MgSO₄), filtered and evaporated to provide crude material that was purified by silica gel chromatography using a gradient from 10% to 50% ethyl acetate in hexanes which provided the racemic intended product.

Part 3: The racemic product of part 2 was separated into the single enantiomers by a normal phase SFC-MS method using an IC column (250×10 mm, 5 μm particle size) sold by Chiral Technologies (pn: 83445), eluting with an isocratic gradient of 16% mobile phase B in mobile phase A at 10 mL/min (Mobile phase A=CO₂, Mobile phase B=methanol (+20 mM NH₃), column temperature=40° C.).

The first enantiomer to elute was 1-[17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (enantiomer 1) (2.2 mg, 47%). ¹H NMR (400 MHz, Chloroform-d) δ 7.48-7.37 (m, 1H), 5.96-5.66 (m, 2H), 5.03-4.38 (m, 2H), 3.96-3.25 (m, 3H), 2.44-2.17 (m, 2H), 2.17-1.73 (m, 4H), 1.61 (s, 1H), 1.48-1.14 (m, 2H), 0.96-0.73 (m, 2H) ppm; ¹⁹F NMR (376 MHz, Chloroform-d) δ−61.47, −61.78, −78.89, −79.25 ppm. ESI-MS m/z calc. 467.13922, found 468.1 (M+1)⁺; Retention time: 1.11 minutes (LC Method Q).

The second enantiomer to elute was 1-[17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]ethanone (enantiomer 2) (2.4 mg, 52%). ¹H NMR (400 MHz, Chloroform-d) δ 7.50-7.35 (m, 1H), 5.90-5.62 (m, 2H), 4.91-4.39 (m, 2H), 3.96-3.23 (m, 3H), 2.41-2.20 (m, 2H), 2.16-1.72 (m, 4H), 1.70-1.60 (m, 1H), 1.46-1.18 (m, 2H), 0.99-0.79 (m, 2H) ppm; ¹⁹F NMR (376 MHz, Chloroform-d) δ−61.47, −61.78, −78.88, −79.25 ppm. ESI-MS m/z calc. 467.13922, found 468.1 (M+1)⁺; Retention time: 1.11 minutes (LC Method Q).

### Example 10: Preparation of [17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (enantiomer 1) (Compound 22) and [17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (Enantiomer 2) (Compound 23)

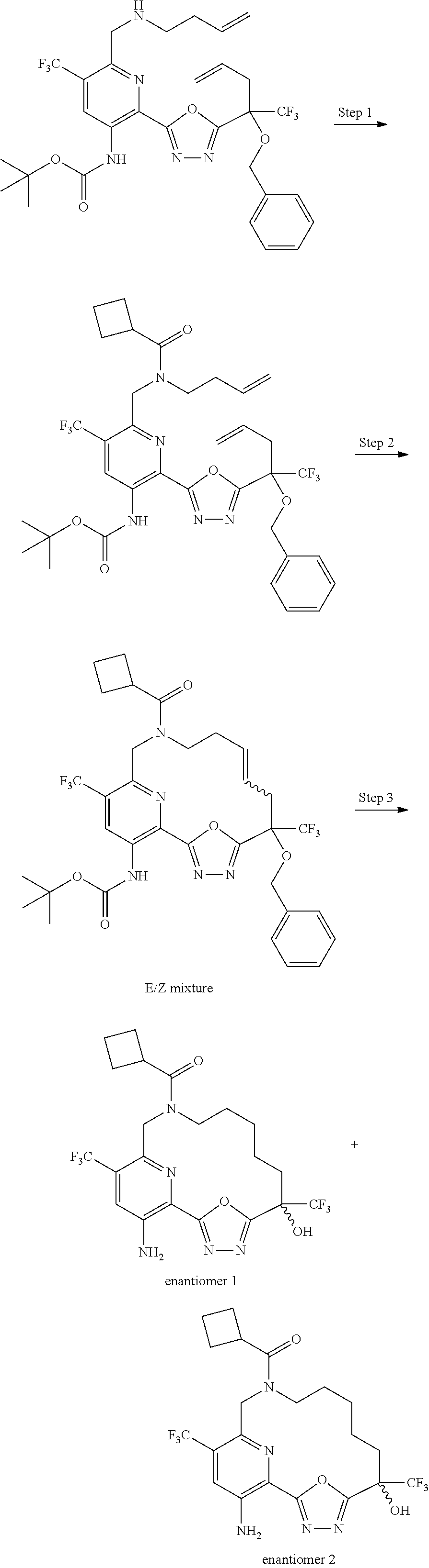 [SMILES: C=CCCNCc1nc(-c2nnc(C(CC=C)(OCc3ccccc3)C(F)(F)F)o2)c(NC(=O)OC(C)(C)C)cc1C(F)(F)F ; C=CCCN(Cc1nc(-c2nnc(C(CC=C)(OCc3ccccc3)C(F)(F)F)o2)c(NC(=O)OC(C)(C)C)cc1C(F)(F)F)C(=O)C1CCC1 ; CC(C)(C)OC(=O)Nc1cc(C(F)(F)F)c2nc1-c1nnc(o1)C(OCc1ccccc1)(C(F)(F)F)CC=CCCN(C(=O)C1CCC1)C2 ; Nc1cc(C(F)(F)F)c2nc1-c1nnc(o1)C(O)(C(F)(F)F)CCCCCN(C(=O)C1CCC1)C2 ; Nc1cc(C(F)(F)F)c2nc1-c1nnc(o1)C(O)(C(F)(F)F)CCCCCN(C(=O)C1CCC1)C2]

### Step 1: tert-Butyl N-[2-[5-[1-benzyloxy-1-(trifluoromethyl)but-3-enyl]-1,3,4-oxadiazol-2-yl]-6-[[but-3-enyl(cyclobutanecarbonyl)amino]methyl]-5-(trifluoromethyl)-3-pyridyl]carbamate

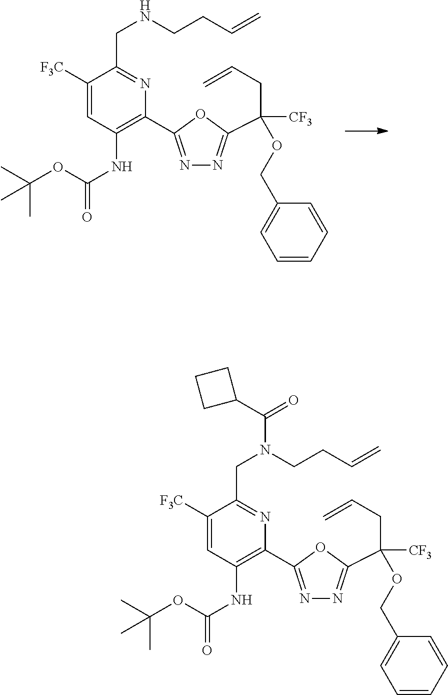 [SMILES: C=CCCNCc1nc(-c2nnc(C(CC=C)(OCc3ccccc3)C(F)(F)F)o2)c(NC(=O)OC(C)(C)C)cc1C(F)(F)F ; C=CCCN(Cc1nc(-c2nnc(C(CC=C)(OCc3ccccc3)C(F)(F)F)o2)c(NC(=O)OC(C)(C)C)cc1C(F)(F)F)C(=O)C1CCC1]

DMF (500 μL) was added to tert-butyl N-[2-[5-[1-benzyloxy-1-(trifluoromethyl)but-3-enyl]-1,3,4-oxadiazol-2-yl]-6-[(but-3-enylamino)methyl]-5-(trifluoromethyl)-3-pyridyl]carbamate (34 mg, 0.05299 mmol) and HATU (40 mg, 0.1052 mmol) and then DIEA (37 μL, 0.2124 mmol) and cyclobutanecarboxylic acid (11 mg, 0.1099 mmol) were added. The mixture was stirred at room temperature for 1 h, then diluted with ether, washed with water and 1 M NaHCO₃, dried (MgSO₄), filtered and evaporated. The residue was purified by silica gel chromatography using a gradient from 0% to 20% ethyl acetate in hexanes to provide tert-butyl N-[2-[5-[1-benzyloxy-1-(trifluoromethyl)but-3-enyl]-1,3,4-oxadiazol-2-yl]-6-[[but-3-enyl(cyclobutanecarbonyl)amino]methyl]-5-(trifluoromethyl)-3-pyridyl]carbamate (32 mg, 83%). ESI-MS m/z calc. 723.2855, found 724.2 (M+1)⁺; Retention time: 0.63 minutes (LC Method T).

### Step 2: tert-Butyl N-[6-benzyloxy-12-(cyclobutanecarbonyl)-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z Mixture)

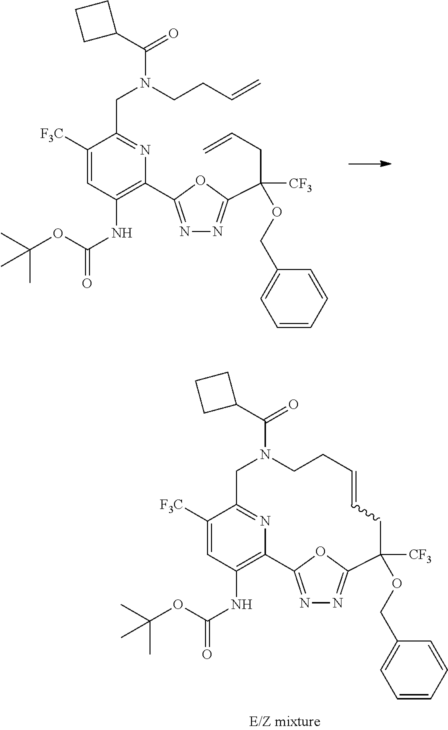 [SMILES: C=CCCN(Cc1nc(-c2nnc(C(CC=C)(OCc3ccccc3)C(F)(F)F)o2)c(NC(=O)OC(C)(C)C)cc1C(F)(F)F)C(=O)C1CCC1 ; CC(C)(C)OC(=O)Nc1cc(C(F)(F)F)c2nc1-c1nnc(o1)C(OCc1ccccc1)(C(F)(F)F)CC=CCCN(C(=O)C1CCC1)C2]

To a 25 mL three neck flask with benzylidene[1,3-bis(2,4,6-trimethylphenyl)-2-imidazolidinylidene]dichloro(tricyclohexylphosphine)ruthenium (5.8 mg, 0.006832 mmol) and toluene (2.35 mL) heated with a 120° C. heat bowl was added a solution of tert-butyl N-[2-[5-[1-benzyloxy-1-(trifluoromethyl)but-3-enyl]-1,3,4-oxadiazol-2-yl]-6-[[but-3-enyl(cyclobutanecarbonyl)amino]methyl]-5-(trifluoromethyl)-3-pyridyl]carbamate (32 mg, 0.04422 mmol) in toluene (2.35 mL) and the mixture heated at 120° C. for 40 min. The solvent was evaporated and the residue purified by silica gel chromatography using a gradient from 0% to 25% ethyl acetate in hexanes to provide tert-butyl N-[6-benzyloxy-12-(cyclobutanecarbonyl)-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (14.5 mg, 46%). ESI-MS m/z calc. 695.2542, found 696.3 (M+1)⁺; Retention time: 0.56 minutes (LC Method T).

### Step 3: [17-Amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (enantiomer 1) (Compound 22) and [17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (Enantiomer 2) (Compound 23)

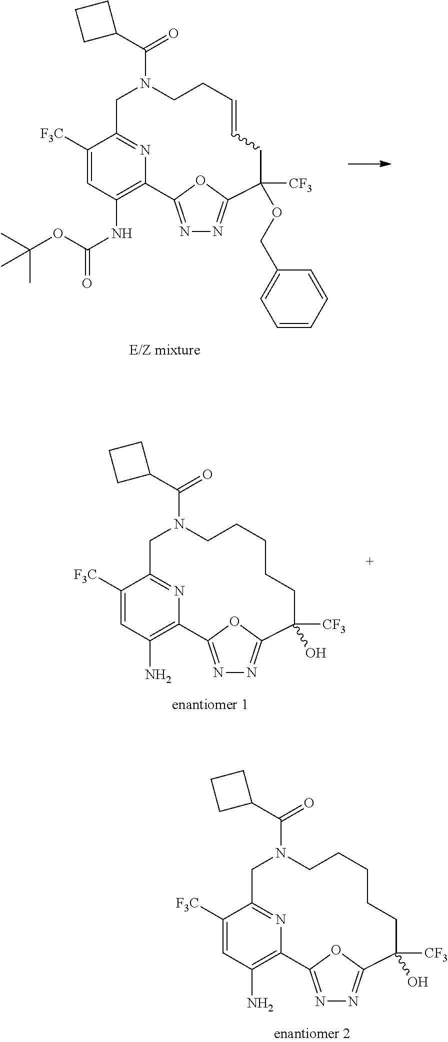 [SMILES: CC(C)(C)OC(=O)Nc1cc(C(F)(F)F)c2nc1-c1nnc(o1)C(OCc1ccccc1)(C(F)(F)F)CC=CCCN(C(=O)C1CCC1)C2 ; Nc1cc(C(F)(F)F)c2nc1-c1nnc(o1)C(O)(C(F)(F)F)CCCCCN(C(=O)C1CCC1)C2 ; Nc1cc(C(F)(F)F)c2nc1-c1nnc(o1)C(O)(C(F)(F)F)CCCCCN(C(=O)C1CCC1)C2]

Part 1: A mixture of tert-butyl N-[6-benzyloxy-12-(cyclobutanecarbonyl)-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z Mixture) (14.5 mg, 0.02084 mmol), 10% palladium on carbon (7 mg, 0.006578 mmol) and acetic acid (290 μL) was stirred at room temperature under 200 psi hydrogen gas for 21 h in a stainless steel vessel. Then, the mixture was filtered and the filtrate was evaporated to provide the intermediate tert-butyl N-[12-(cyclobutanecarbonyl)-6-hydroxy-6,15-bis(trifluoromethyl)-19-oxa-3,4,12,18-tetrazatricyclo[12.3.1.12,5]nonadeca-1(17),2,4,14(18),15-pentaen-17-yl]carbamate (13.5 mg, quant.). ESI-MS m/z calc. 607.22296, found 608.2 (M+1)⁺; Retention time: 0.78 minutes (LC Method T).

Part 2: The residue from part 1 was dissolved in TFA (0.3 mL), triisopropylsilane (15 μL, 0.07322 mmol) and water (15 μL) and stirred for 1 h at room temperature. The solvent was evaporated and the residue was dissolved in ethyl acetate and washed with 1 M NaHCO₃, dried (MgSO₄), filtered and evaporated. The resulting residue was purified by silica gel chromatography using a gradient from 10% to 50% ethyl acetate in hexanes which provided the racemic intended product.

Part 3: The residue from part 2 was separated into the single enantiomers by a normal phase SFC-MS method using an IC column (250×10 mm, 5 μm particle size) sold by Chiral Technologies (pn: 83445), eluting with an isocratic gradient of 16% mobile phase B in mobile phase A at 10 mL/min (Mobile phase A=CO₂, Mobile phase B=MeOH (+20 mM NH₃), column temperature=40° C.). The first enantiomer to elute was isolated as [17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (enantiomer 1) (3.1 mg, 59%). ¹H NMR (400 MHz, Chloroform-d) δ 7.49-7.35 (m, 1H), 5.92-5.61 (m, 2H), 4.90-4.38 (m, 2H), 3.93-2.86 (m, 4H), 2.46-2.17 (m, 4H), 2.15-2.06 (m, 2H), 1.89 (m, 6H), 1.44-1.14 (m, 1H), 0.96-0.78 (m, 1H) ppm; ¹⁹F NMR (376 MHz, Chloroform-d) δ−61.40, −61.86, −78.85, −79.38 ppm. ESI-MS m/z calc. 507.1705, found 508.1 (M+1)⁺; Retention time: 1.35 minutes (LC Method Q).

The second enantiomer to elute was isolated as [17-amino-6-hydroxy-6,15-bis(trifluoromethyl)-19-oxa-3,4,12,18-tetrazatricyclo[12.3.1.12,5]nonadeca-1(18),2,4,14,16-pentaen-12-yl]-cyclobutyl-methanone (enantiomer 2) (2.6 mg, 49%). ¹H NMR (400 MHz, Chloroform-d) δ 7.48-7.33 (m, 1H), 5.89-5.58 (m, 2H), 4.86-4.42 (m, 2H), 3.94-2.87 (m, 4H), 2.44-2.18 (m, 4H), 2.16-2.03 (m, 2H), 2.01-1.69 (m, 6H), 1.44-1.28 (m, 1H), 0.98-0.79 (m, 1H) ppm; ¹⁹F NMR (376 MHz, Chloroform-d) δ−61.40, −61.86, −78.84, −79.38 ppm. ESI-MS m/z calc. 507.1705, found 508.1 (M+1)⁺; Retention time: 1.35 minutes (LC Method Q).

### Example 11: Preparation of 20-amino-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-6-ol (Hydrochloride Salt)

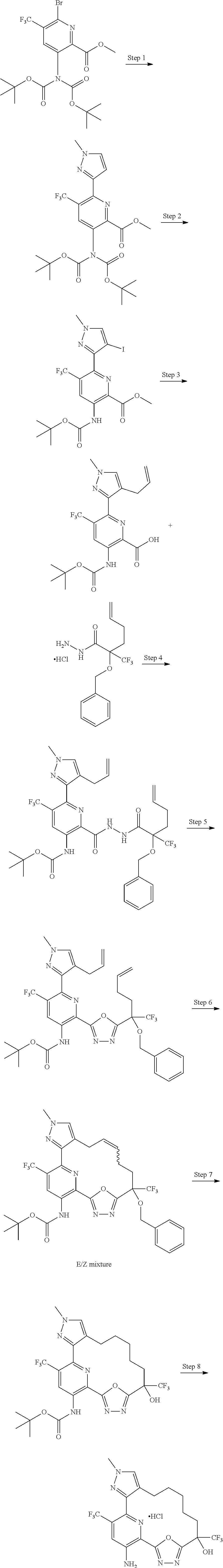

### Step 1: Methyl 3-[bis(tert-butoxycarbonyl)amino]-6-(1-methylpyrazol-3-yl)-5-(trifluoromethyl)pyridine-2-carboxylate

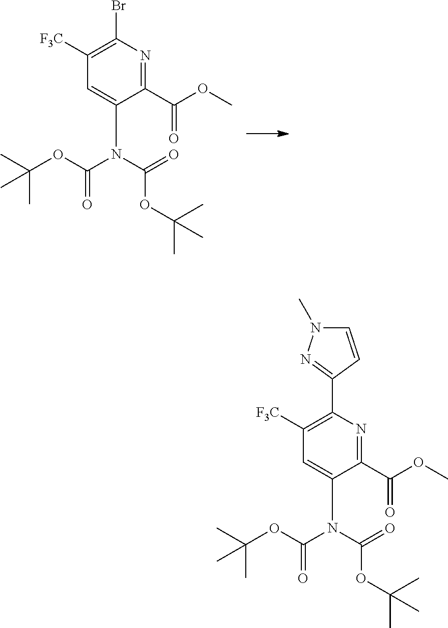 [SMILES: COC(=O)c1nc(Br)c(C(F)(F)F)cc1N(C(=O)OC(C)(C)C)C(=O)OC(C)(C)C ; COC(=O)c1nc(-c2ccn(C)n2)c(C(F)(F)F)cc1N(C(=O)OC(C)(C)C)C(=O)OC(C)(C)C]

To a solution of methyl 3-[bis(tert-butoxycarbonyl)amino]-6-bromo-5-(trifluoromethyl)pyridine-2-carboxylate (1 g, 2.003 mmol) in DMA (10 mL) in a microwave vial was added 1-methyl-3-(4,4,5,5-tetramethyl-1,3,2-dioxaborolan-2-yl)pyrazole (684 mg, 3.287 mmol) and purged with nitrogen for 5 min. Then Pd(dppf)Cl₂ (174 mg, 0.2131 mmol) was added followed by potassium carbonate (3.2 mL of 2 M, 6.4 mmol). The microwave vial was capped and heated at 100° C. in a microwave synthesizer for 30 min. The reaction mixture was cooled to room temperature and water was added. The mixture was extracted with ethyl acetate and combined organic layers washed with brine. The organics were separated, dried over sodium sulfate, filtered and evaporated. The resultant brown residue was purified by silica gel chromatography using a gradient from 100% hexanes to 100% ethyl acetate to afford as a light brown solid, methyl 3-[bis(tert-butoxycarbonyl)amino]-6-(1-methylpyrazol-3-yl)-5-(trifluoromethyl)pyridine-2-carboxylate (762 mg, 76%). ¹H NMR (400 MHz, DMSO-d₆) δ 8.56 (s, 1H), 7.84 (d, J=2.3 Hz, 1H), 6.70 (d, J=2.2 Hz, 1H), 3.94 (s, 3H), 3.88 (s, 3H), 1.35 (s, 18H) ppm. ESI-MS m/z calc. 500.18826, found 501.2 (M+1)⁺; Retention time: 0.47 minutes (LC Method R).

### Step 2: Methyl 3-(tert-butoxycarbonylamino)-6-(4-iodo-1-methyl-pyrazol-3-yl)-5-(trifluoromethyl)pyridine-2-carboxylate

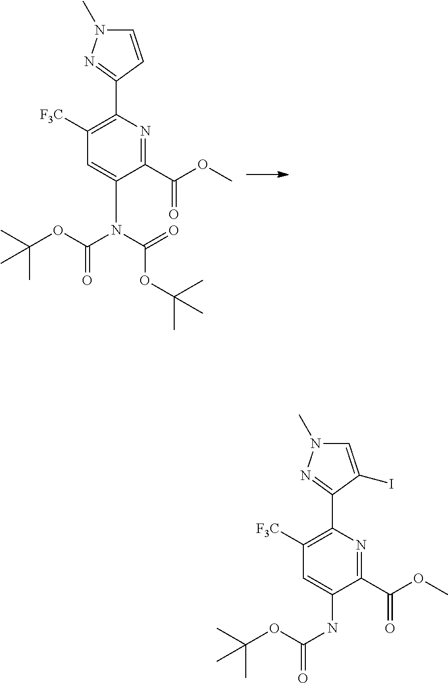 [SMILES: COC(=O)c1nc(-c2ccn(C)n2)c(C(F)(F)F)cc1N(C(=O)OC(C)(C)C)C(=O)OC(C)(C)C ; COC(=O)c1nc(-c2nn(C)cc2I)c(C(F)(F)F)cc1NC(=O)OC(C)(C)C]

To a solution of methyl 3-[bis(tert-butoxycarbonyl)amino]-6-(1-methylpyrazol-3-yl)-5-(trifluoromethyl)pyridine-2-carboxylate (500 mg, 0.9991 mmol) in dichloromethane (10 mL) was added TFA (250 μL, 3.245 mmol) followed by N-Iodosuccinimide (200 mg, 0.8890 mmol). After stirring the reaction mixture for 2 h at 23° C., the mixture was diluted with dichloromethane, washed with water, aqueous NaHCO₃ and aqueous sodium thiosulfate. The organic layer was dried and concentrated. The resultant brown residue was purified by silica gel chromatography using a gradient from 100% hexanes to 100% ethyl acetate to afford as a brown solid, methyl 3-(tert-butoxycarbonylamino)-6-(4-iodo-1-methyl-pyrazol-3-yl)-5-(trifluoromethyl)pyridine-2-carboxylate (423 mg, 80%). ESI-MS m/z calc. 526.0325, found 527.0 (M+1)⁺; Retention time: 0.56 minutes (LC Method R).

### Step 3: 6-(4-Allyl-1-methyl-pyrazol-3-yl)-3-(tert-butoxycarbonylamino)-5-(trifluoromethyl)pyridine-2-carboxylic Acid

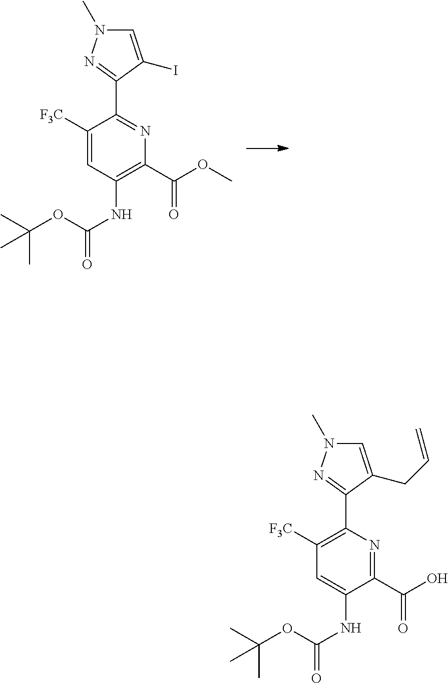 [SMILES: COC(=O)c1nc(-c2nn(C)cc2I)c(C(F)(F)F)cc1NC(=O)OC(C)(C)C ; C=CCc1cn(C)nc1-c1nc(C(=O)O)c(NC(=O)OC(C)(C)C)cc1C(F)(F)F]

To a solution of methyl 3-(tert-butoxycarbonylamino)-6-(4-iodo-1-methyl-pyrazol-3-yl)-5-(trifluoromethyl)pyridine-2-carboxylate (423 mg, 0.8038 mmol) in DMA (5 mL) in a microwave vial was added 2-allyl-4,4,5,5-tetramethyl-1,3,2-dioxaborolane (230 μL, 1.226 mmol) and purged with nitrogen for 5 min. Then Pd(dppf)Cl₂ (71 mg, 0.08694 mmol) followed by potassium carbonate (1.3 mL of 2 M, 2.600 mmol) were added. The microwave vial was capped and heated at 100° C. in a microwave synthesizer for 30 min. The reaction mixture was cooled to room temperature and filtered through Celite, then the Celite was washed with ethyl acetate. The filtrate was washed with brine, dried over sodium sulfate, filtered and concentrated. The resultant brown residue was purified by silica gel chromatography using a gradient from 100% hexanes to 100% ethyl acetate giving as light brown solid, 6-(4-allyl-1-methyl-pyrazol-3-yl)-3-(tert-butoxycarbonylamino)-5-(trifluoromethyl)pyridine-2-carboxylic acid (296 mg, 86%). ESI-MS m/z calc. 426.1515, found 427.2 (M+1)⁺; Retention time: 0.53 minutes (LC Method R). The product was contaminated with inseparable side product, 3-(tert-butoxycarbonylamino)-6-(4-iodo-1-methyl-pyrazol-3-yl)-5-(trifluoromethyl)pyridine-2-carboxylic acid; ESI-MS m/z calc. 512.01685, found 512.94 (M+1)⁺; Retention time: 0.51 minutes (LC Method R).

### Step 4: tert-Butyl N-[6-(4-allyl-1-methyl-pyrazol-3-yl)-2-[[[2-benzyloxy-2-(trifluoromethyl)hex-5-enoyl]amino]carbamoyl]-5-(trifluoromethyl)-3-pyridyl]carbamate

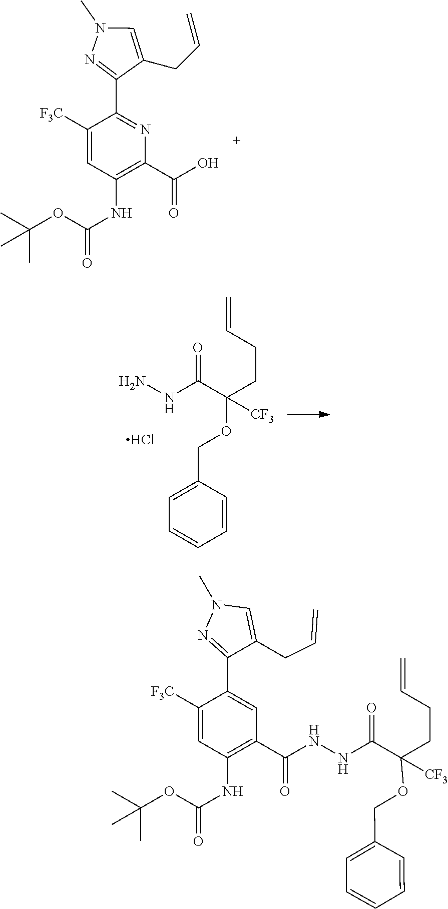

To a solution of 6-(4-allyl-1-methyl-pyrazol-3-yl)-3-(tert-butoxycarbonylamino)-5-(trifluoromethyl)pyridine-2-carboxylic acid (280 mg, 0.6567 mmol) [containing inseparable side product, 3-(tert-butoxycarbonylamino)-6-(4-iodo-1-methyl-pyrazol-3-yl)-5-(trifluoromethyl)pyridine-2-carboxylic acid] in DMF (5 mL) was added 2-benzyloxy-2-(trifluoromethyl)hex-5-enehydrazide (hydrochloride salt) (192 mg, 0.5668 mmol), DIEA (350 μL, 2.009 mmol), followed by HATU (301 mg, 0.7916 mmol). The reaction mixture was stirred at room temperature for 18 h. Water was added to the mixture and extracted with ethyl acetate. The combined organics washed with brine, and dried over MgSO₄, filtered, and concentrated. The resultant brown residue was purified by silica gel chromatography using a gradient from 100% hexanes to 30% ethyl acetate in hexanes which gave as a pale yellow viscous oil, tert-butyl N-[6-(4-allyl-1-methyl-pyrazol-3-yl)-2-[[[2-benzyloxy-2-(trifluoromethyl)hex-5-enoyl]amino]carbamoyl]-5-(trifluoromethyl)-3-pyridyl]carbamate (117 mg, 25%). ESI-MS m/z calc. 710.26514, found 711.26 (M+1)⁺; Retention time: 0.57 minutes (LC Method T).

### Step 5: tert-Butyl N-[6-(4-allyl-1-methyl-pyrazol-3-yl)-2-[5-[1-benzyloxy-1-(trifluoromethyl)pent-4-enyl]-1,3,4-oxadiazol-2-yl]-5-(trifluoromethyl)-3-pyridyl]carbamate

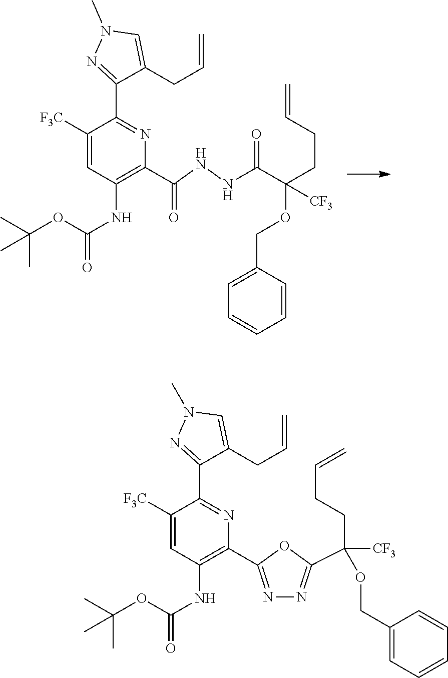 [SMILES: C=CCCC(OCc1ccccc1)(C(=O)NNC(=O)c1nc(-c2nn(C)cc2CC=C)c(C(F)(F)F)cc1NC(=O)OC(C)(C)C)C(F)(F)F ; C=CCCC(OCc1ccccc1)(c1nnc(-c2nc(-c3nn(C)cc3CC=C)c(C(F)(F)F)cc2NC(=O)OC(C)(C)C)o1)C(F)(F)F]

To a solution of tert-butyl N-[6-(4-allyl-1-methyl-pyrazol-3-yl)-2-[[[2-benzyloxy-2-(trifluoromethyl)hex-5-enoyl]amino]carbamoyl]-5-(trifluoromethyl)-3-pyridyl]carbamate (117 mg, 0.1646 mmol) in CH₃CN (10 mL) was added DIEA (100 μL, 0.5741 mmol) and was heated to 70° C., then 4-methylbenzenesulfonyl chloride (45 mg, 0.2360 mmol) was added in 3 portions (85 mg each portion in 10 min intervals). The resulted mixture was heated at 70° C. for 16 hours. The reaction mixture was cooled and quenched with saturated solution of sodium bicarbonate and extracted with ethyl acetate. The combined organic layer was washed with brine, dried over sodium sulfate, filtered, solvent evaporated. The resultant brown residue was purified by silica gel chromatography using a gradient from 100% hexanes to 30% ethyl acetate which gave tert-butyl N-[6-(4-allyl-1-methyl-pyrazol-3-yl)-2-[5-[1-benzyloxy-1-(trifluoromethyl)pent-4-enyl]-1,3,4-oxadiazol-2-yl]-5-(trifluoromethyl)-3-pyridyl]carbamate (78 mg, 68%). ¹H NMR (400 MHz, Chloroform-d) δ 10.16 (s, 1H), 9.36 (s, 1H), 7.38 (dd, J=6.7, 3.0 Hz, 2H), 7.27-7.23 (m, 4H), 5.86 (m, 1H), 5.73 (m, 1H), 5.04-4.89 (m, 3H), 4.89-4.80 (m, 2H), 4.67 (d, J=10.6 Hz, 1H), 3.93 (s, 3H), 3.36 (dd, J=6.5, 1.7 Hz, 2H), 2.54-2.41 (m, 2H), 2.36-2.29 (m, 1H), 2.25-2.14 (m, 1H), 1.57 (s, 9H) ppm. ESI-MS m/z calc. 692.2546, found 693.3 (M+1)⁺; Retention time: 0.69 minutes (LC Method T).

### Step 6: tert-Butyl N-[6-(benzyloxy)-14-methyl-6,18-bis(trifluoromethyl)-22-oxa-3,4,14,15,21-pentaazatetracyclo[15.3.1.12,5.012,16]docosa-1(21),2,4,9,12,15,17,19-octaen-20-yl]carbamate (E/Z Mixture)

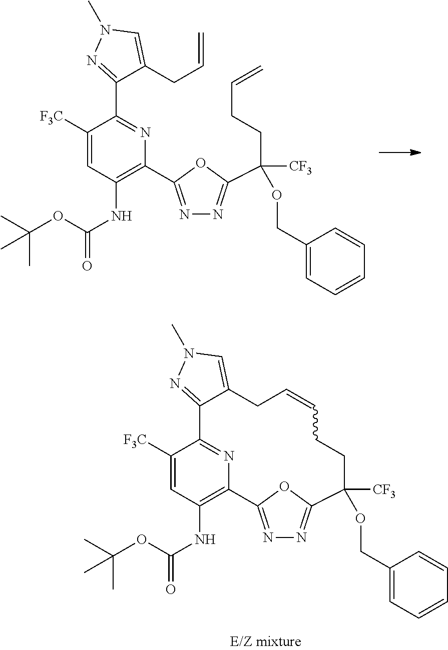 [SMILES: C=CCCC(OCc1ccccc1)(c1nnc(-c2nc(-c3nn(C)cc3CC=C)c(C(F)(F)F)cc2NC(=O)OC(C)(C)C)o1)C(F)(F)F ; Cn1cc2c(n1)-c1nc(c(NC(=O)OC(C)(C)C)cc1C(F)(F)F)-c1nnc(o1)C(OCc1ccccc1)(C(F)(F)F)CCC=CC2]

To a degassed solution of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (13 mg, 0.03042 mmol) in 1,2-dichloroethane (15 mL) was added a degassed solution of tert-butyl N-[6-(4-allyl-1-methyl-pyrazol-3-yl)-2-[5-[1-benzyloxy-1-(trifluoromethyl)pent-4-enyl]-1,3,4-oxadiazol-2-yl]-5-(trifluoromethyl)-3-pyridyl]carbamate (78 mg, 0.1126 mmol) in 1,2-dichloroethane (15 mL) slowly dropwise under a stream of N₂ flow bubbling through the solution over 30 min and the reaction mixture was heated at 60° C. for 5 h. The reaction mixture was cooled to room temperature and solvents removed by concentration under reduced pressure. The resultant brown residue was purified by silica gel chromatography using a gradient from 100% hexanes to 30% ethyl acetate in hexanes which gave tert-butyl N-[6-(benzyloxy)-14-methyl-6,18-bis(trifluoromethyl)-22-oxa-3,4,14,15,21-pentaazatetracyclo[15.3.1.12,5.012,16]docosa-1(21),2,4,9,12,15,17,19-octaen-20-yl]carbamate (E/Z mixture) (26 mg, 35%). ESI-MS m/z calc. 664.22327, found 665.2 (M+1)⁺; Retention time: 0.6 minutes (LC Method T).

### Step 7: tert-Butyl N-[6-hydroxy-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-20-yl]carbamate

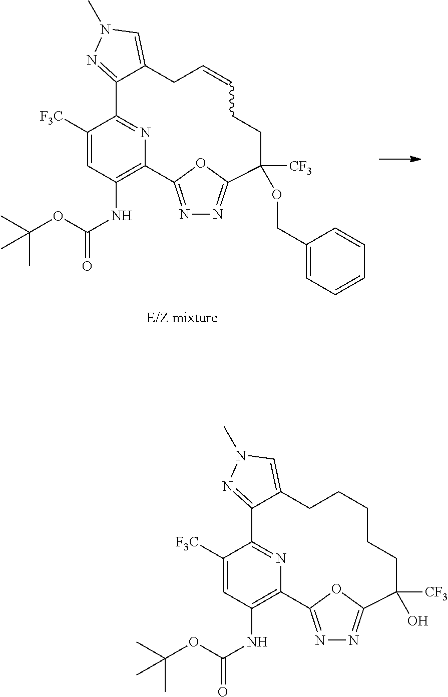 [SMILES: Cn1cc2c(n1)-c1nc(c(NC(=O)OC(C)(C)C)cc1C(F)(F)F)-c1nnc(o1)C(OCc1ccccc1)(C(F)(F)F)CCC=CC2 ; Cn1cc2c(n1)-c1nc(c(NC(=O)OC(C)(C)C)cc1C(F)(F)F)C1=NN=C(O1)C(O)(C(F)(F)F)CCCCC2]

To a solution of tert-butyl N-[6-(benzyloxy)-14-methyl-6,18-bis(trifluoromethyl)-22-oxa-3,4,14,15,21-pentaazatetracyclo[15.3.1.12,5.012,16]docosa-1(21),2,4,9,12,15,17,19-octaen-20-yl]carbamate (E/Z mixture) (26 mg, 0.03912 mmol) in acetic acid (5 mL) was added Pd/C (16 mg of 10% w/w, 50% wet, 0.01503 mmol) in a round bottom flask equipped with a H₂ balloon using a 3-way adaptor. The mixture was subjected to vacuum and backfilled with nitrogen gas three times then subjected to vacuum again. The flask was filled with hydrogen gas then stirred the mixture for 15 hours. The mixture was then subjected to vacuum and backfilled with nitrogen gas three times then diluted with ethyl acetate and filtered over Celite. The filtrate was concentrated and dried under high vac to afford tert-butyl N-[6-hydroxy-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-20-yl]carbamate (20 mg, 89%). ESI-MS m/z calc. 576.19196, found 577.1 (M+1)⁺; Retention time: 0.42 minutes (LC Method T).

### Step 8: 20-Amino-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-6-ol (Hydrochloride Salt) (Compound 24)

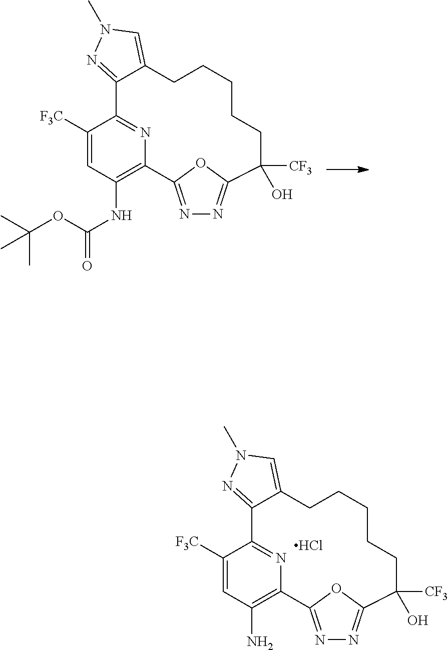 [SMILES: Cn1cc2c(n1)-c1nc(c(NC(=O)OC(C)(C)C)cc1C(F)(F)F)C1=NN=C(O1)C(O)(C(F)(F)F)CCCCC2 ; Cn1cc2c(n1)-c1nc(c(N)cc1C(F)(F)F)C1=NN=C(O1)C(O)(C(F)(F)F)CCCCC2 ; Cl]

tert-Butyl N-[6-hydroxy-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-20-yl]carbamate (20 mg) was dissolved in pre-made solution of TFA (100 μL, 1.298 mmol) and dichloromethane (400 μL) and the reaction was stirred at room temperature for about 1 h. The solvents were removed, and the residue was dissolved in DMSO (1 mL) and the mixture was purified by a reverse phase HPLC using a mobile gradient from 30% to 99% acetonitrile in water (+5 mM HCl) over 15 minutes which gave as a colorless solid and as a racemic mixture, 20-amino-14-methyl-6,18-bis(trifluoromethyl)-22-oxa-3,4,14,15,21-pentaazatetracyclo[15.3.1.12,5.012,16]docosa-1(21),2,4,12,15,17,19-heptaen-6-ol (hydrochloride salt) (1 mg, 5%). ESI-MS m/z calc. 476.13956, found 477.1 (M+1)⁺; Retention time: 0.97 minutes (LC Method J).

### Example 12: Preparation of 17-amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 1, Enantiomer 1) (Compound 25), 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 1, enantiomer 2) (Compound 26), 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 2, enantiomer 3) (Compound 27), and 17-amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 2, Enantiomer 4) (Compound 28)

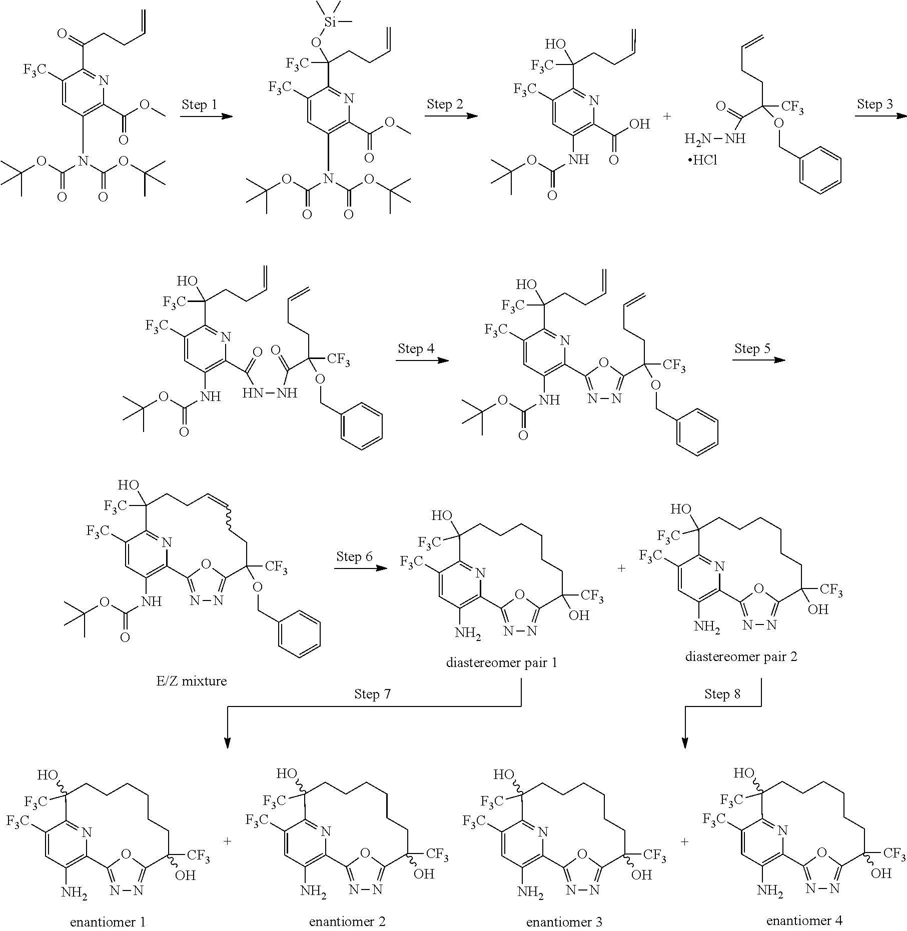

### Step 1: Methyl 3-[bis(tert-butoxycarbonyl)amino]-5-(trifluoromethyl)-6-[1-(trifluoromethyl)-1-trimethylsilyloxy-pent-4-enyl]pyridine-2-carboxylate

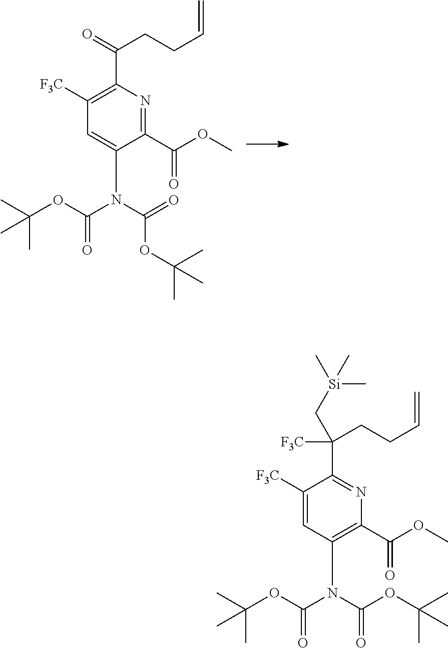 [SMILES: C=CCCC(=O)c1nc(C(=O)OC)c(N(C(=O)OC(C)(C)C)C(=O)OC(C)(C)C)cc1C(F)(F)F ; C=CCCC(C[Si](C)(C)C)(c1nc(C(=O)OC)c(N(C(=O)OC(C)(C)C)C(=O)OC(C)(C)C)cc1C(F)(F)F)C(F)(F)F]

To a stirred solution of methyl 3-[bis(tert-butoxycarbonyl)amino]-6-pent-4-enoyl-5-(trifluoromethyl)pyridine-2-carboxylate (500 mg, 0.9951 mmol) in THF (5 mL) was added trimethyl(trifluoromethyl)silane (1.2 g, 8.439 mmol) at 0° C. Then, tetrabutylammonium fluoride (50 μL of 1 M, 0.05 mmol, 1 M solution in THF) was added. The reaction mixture was slowly warmed to room temperature and stirred for 15 hours. After this time, 5 mL of water was added and extracted with ethyl acetate (3×10 mL). Organic phases were combined, dried over sodium sulfate, filtered and evaporated. The residue was purified by reverse phase chromatography using a mobile gradient from 40% acetonitrile in water to 100% acetonitrile over 6 minutes provided methyl 3-[bis(tert-butoxycarbonyl)amino]-5-(trifluoromethyl)-6-[1-(trifluoromethyl)-1-trimethylsilyloxy-pent-4-enyl]pyridine-2-carboxylate (265 mg, 41%). ESI-MS m/z calc. 644.2352, found 645.3 (M+1)⁺; Retention time: 1.98 minutes (LC Method M).

### Step 2: 3-(tert-Butoxycarbonylamino)-6-[l-hydroxy-1-(trifluoromethyl)pent-4-enyl]-5-(trifluoromethyl)pyridine-2-carboxylic Acid

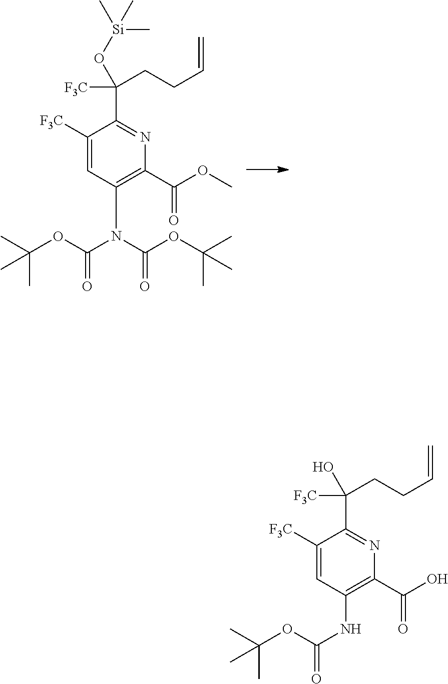 [SMILES: C=CCCC(O[Si](C)(C)C)(c1nc(C(=O)OC)c(N(C(=O)OC(C)(C)C)C(=O)OC(C)(C)C)cc1C(F)(F)F)C(F)(F)F ; C=CCCC(O)(c1nc(C(=O)O)c(NC(=O)OC(C)(C)C)cc1C(F)(F)F)C(F)(F)F]

To a stirred solution of methyl 3-[bis(tert-butoxycarbonyl)amino]-5-(trifluoromethyl)-6-[1-(trifluoromethyl)-1-trimethylsilyloxy-pent-4-enyl]pyridine-2-carboxylate (265 mg, 0.4111 mmol) in THF (4 mL), H₂O (1 mL) and acetonitrile (2 mL) was added LiOH (30 mg, 1.253 mmol) in two portions (5 min from each other). After 50 minutes, organic solvents were evaporated, 5 mL of HCl (10%) was added and extracted with ethyl acetate (3×10 mL). Organic phases were combined, dried over sodium sulfate, filtered and concentrated. The residue was purified by reverse phase chromatography using a mobile gradient from 30% acetonitrile in water to 100% acetonitrile over 7 minutes provided 3-(tert-butoxycarbonylamino)-6-[1-hydroxy-1-(trifluoromethyl)pent-4-enyl]-5-(trifluoromethyl)pyridine-2-carboxylic acid (131 mg, 70%). ¹H NMR (400 MHz, CDCl₃) δ 10.14 (s, 1H), 9.52 (d, J=3.0 Hz, 1H), 7.21 (bd, J=7.5, 4.4 Hz, 1H), 6.03-5.42 (m, 1H), 5.21-4.70 (m, 2H), 3.29 (s, 1H), 2.53-2.45 (m, 1H), 2.25-2.06 (m, 2H), 1.97-1.88 (m, 1H), 1.56 (s, 9H) ppm. ESI-MS m/z calc. 458.12766, found 459.1 (M+1)⁺; Retention time: 1.48 minutes (LC Method J).

### Step 3: tert-Butyl N-[2-[[[2-benzyloxy-2-(trifluoromethyl)hex-5-enoyl]amino]carbamoyl]-6-[1-hydroxy-1-(trifluoromethyl)pent-4-enyl]-5-(trifluoromethyl)-3-pyridyl]carbamate

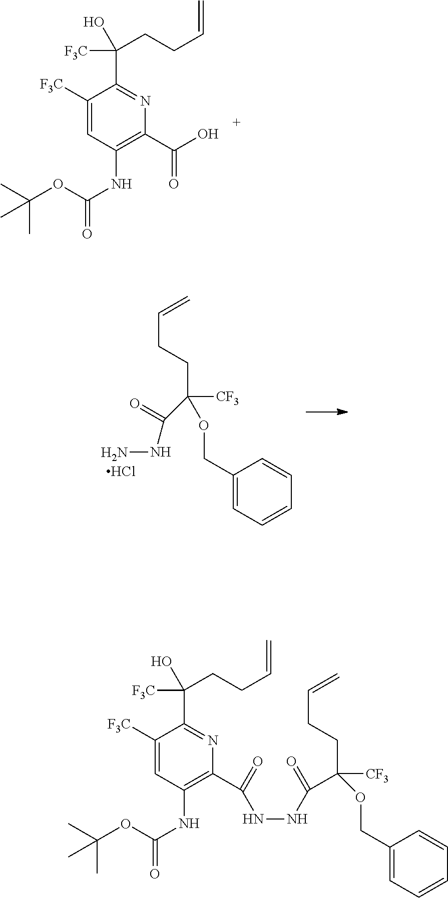 [SMILES: C=CCCC(O)(c1nc(C(=O)O)c(NC(=O)OC(C)(C)C)cc1C(F)(F)F)C(F)(F)F ; Cl ; C=CCCC(OCc1ccccc1)(C(=O)NN)C(F)(F)F ; C=CCCC(OCc1ccccc1)(C(=O)NNC(=O)c1nc(C(O)(CCC=C)C(F)(F)F)c(C(F)(F)F)cc1NC(=O)OC(C)(C)C)C(F)(F)F]

To a solution of 3-(tert-butoxycarbonylamino)-6-[1-hydroxy-1-(trifluoromethyl)pent-4-enyl]-5-(trifluoromethyl)pyridine-2-carboxylic acid (110 mg, 0.2400 mmol) and 2-benzyloxy-2-(trifluoromethyl)hex-5-enehydrazide (hydrochloride salt) (89.5 mg, 0.2642 mmol) in NMP (5 mL) was added DIEA (93 mg, 0.7196 mmol), followed by HATU (137 mg, 0.3603 mmol). The reaction mixture was stirred at room temperature for 1 h. The mixture was diluted with water (100 mL) and extracted with ethyl acetate (3×50 mL). The organic phases were combined and dried over MgSO₄, filtered, and concentrated. Purification by silica gel chromatography using a gradient from 0% to 15% ethyl acetate in hexanes provided tert-butyl N-[2-[[[2-benzyloxy-2-(trifluoromethyl)hex-5-enoyl]amino]carbamoyl]-6-[1-hydroxy-1-(trifluoromethyl)pent-4-enyl]-5-(trifluoromethyl)-3-pyridyl]carbamate (150 mg, 84%). ESI-MS m/z calc. 742.2413, found 743.2 (M+1)⁺; Retention time: 1.59 minutes (LC Method M).

### Step 4: tert-Butyl N-[2-[5-[1-benzyloxy-1-(trifluoromethyl)pent-4-enyl]-1,3,4-oxadiazol-2-yl]-6-[1-hydroxy-1-(trifluoromethyl)pent-4-enyl]-5-(trifluoromethyl)-3-pyridyl]carbamate

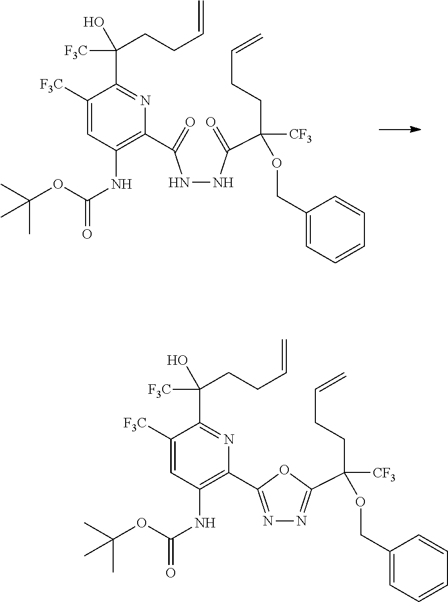 [SMILES: C=CCCC(OCc1ccccc1)(C(=O)NNC(=O)c1nc(C(O)(CCC=C)C(F)(F)F)c(C(F)(F)F)cc1NC(=O)OC(C)(C)C)C(F)(F)F ; C=CCCC(O)(c1nc(-c2nnc(C(CCC=C)(OCc3ccccc3)C(F)(F)F)o2)c(NC(=O)OC(C)(C)C)cc1C(F)(F)F)C(F)(F)F]

A solution of tert-butyl N-[2-[[[2-benzyloxy-2-(trifluoromethyl)hex-5-enoyl]amino]carbamoyl]-6-[1-hydroxy-1-(trifluoromethyl)pent-4-enyl]-5-(trifluoromethyl)-3-pyridyl]carbamate (240 mg, 0.3232 mmol) and DIEA (104.5 mg, 0.8086 mmol) in acetonitrile (5 mL) was heated to 50° C., then p-TsCl (92.5 mg, 0.4852 mmol) was added. The resulted mixture was heated at 70° C. for 2 h and cooled to room temperature. The reaction mixture was quenched with saturated solution of sodium bicarbonate (10 mL) and extracted with ethyl acetate (3×20 mL). Organic layers combined and washed with brine (30 mL), dried over sodium sulfate, filtered and solvents evaporated. Purification by silica gel chromatography using 90:10 hexanes and ethyl acetate provided tert-butyl N-[2-[5-[1-benzyloxy-1-(trifluoromethyl)pent-4-enyl]-1,3,4-oxadiazol-2-yl]-6-[1-hydroxy-1-(trifluoromethyl)pent-4-enyl]-5-(trifluoromethyl)-3-pyridyl]carbamate (145 mg, 62%). ESI-MS m/z calc. 724.2307, found 725.2 (M+1)⁺; Retention time: 1.93 minutes (LC Method M).

### Step 5: tert-Butyl N-[6-benzyloxy-13-hydroxy-6,13,15-tris(trifluoromethyl)-19-oxa-3,4,18-triazatricyclo[12.3.1.12,5]nonadeca-1(17),2,4,9,14(18),15-hexaen-17-yl]carbamate (E/Z Mixture)

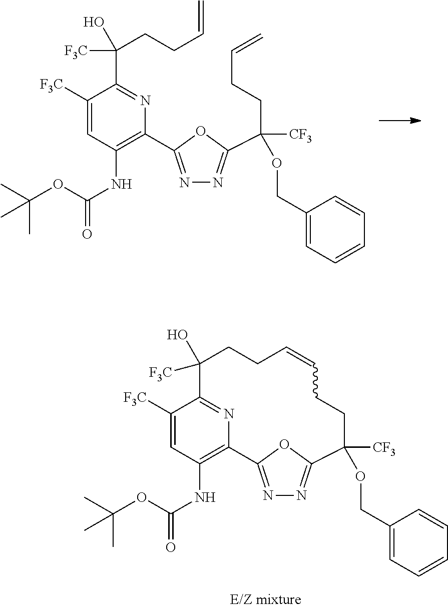 [SMILES: C=CCCC(O)(c1nc(-c2nnc(C(CCC=C)(OCc3ccccc3)C(F)(F)F)o2)c(NC(=O)OC(C)(C)C)cc1C(F)(F)F)C(F)(F)F ; CC(C)(C)OC(=O)Nc1cc(C(F)(F)F)c2nc1C1=NN=C(O1)C(OCc1ccccc1)(C(F)(F)F)CCC=CCCC2(O)C(F)(F)F]

To a solution of tert-butyl N-[2-[5-[1-benzyloxy-1-(trifluoromethyl)pent-4-enyl]-1,3,4-oxadiazol-2-yl]-6-[1-hydroxy-1-(trifluoromethyl)pent-4-enyl]-5-(trifluoromethyl)-3-pyridyl]carbamate (280 mg, 0.3864 mmol) in toluene (100 mL) at 90° C. was added [1,3-bis-(2-tolyl)-2-imidazolidinylidene]dichloro(2-isopropoxybenzylidene)ruthenium(II) (32 mg, 0.05609 mmol) while nitrogen gas bubbled in the solution through a rubber septum. The mixture was heated at 110° C. for 3 hours. The solvent was evaporated and the residue was purified by silica gel chromatography using 90:10 hexanes and ethyl acetate) which gave two separated isomeric mixtures of the product: tert-butyl N-[6-benzyloxy-13-hydroxy-6,13,15-tris(trifluoromethyl)-19-oxa-3,4,18-triazatricyclo[12.3.1.12,5]nonadeca-1(17),2,4,9,14(18),15-hexaen-17-yl]carbamate (E/Z mixture) (isomer 1, 31 mg, 12%, ESI-MS m/z calc. 696.1994, found 697.3 (M+1)⁺; Retention time: 1.58 minutes (LC Method M) and isomer 2, 30 mg, 11%, ESI-MS m/z calc. 696.1994, found 697.2 (M+1)⁺; Retention time: 1.66 minutes (LC Method M). Because of the scale and some impurity of the compounds, both isomers were mixed and taken to the next step.

### Step 6: 17-Amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 1) and 17-amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 2)

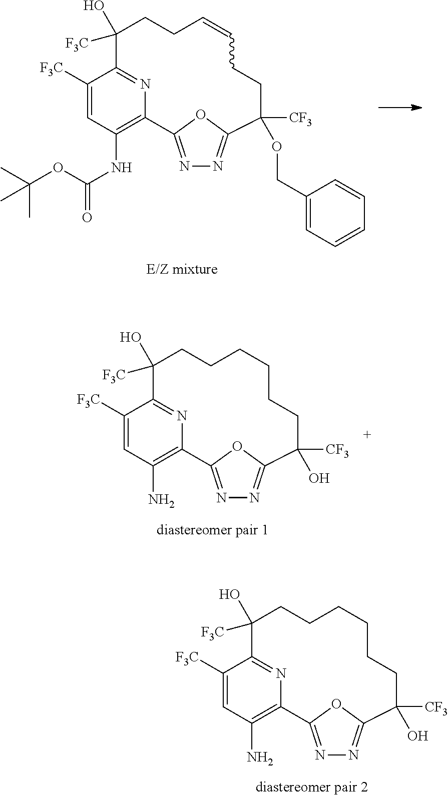 [SMILES: CC(C)(C)OC(=O)Nc1cc(C(F)(F)F)c2nc1C1=NN=C(O1)C(OCc1ccccc1)(C(F)(F)F)CCC=CCCC2(O)C(F)(F)F ; Nc1cc(C(F)(F)F)c2nc1C1=NN=C(O1)C(O)(C(F)(F)F)CCCCCCC2(O)C(F)(F)F ; Nc1cc(C(F)(F)F)c2nc1C1=NN=C(O1)C(O)(C(F)(F)F)CCCCCCC2(O)C(F)(F)F]

tert-Butyl N-[6-benzyloxy-13-hydroxy-6,13,15-tris(trifluoromethyl)-19-oxa-3,4,18-triazatricyclo[12.3.1.12,5]nonadeca-1(17),2,4,9,14(18),15-hexaen-17-yl]carbamate (E/Z mixture) (61 mg, 0.08757 mmol) was dissolved in acetic acid (2 mL) and transferred to a high pressure reactor. Next, Pd/C (19 mg, 10% w/w, 0.008927 mmol, 50% wet) was added and the Parr reactor was evacuated and filled with nitrogen gas 3 times and finally filled with hydrogen gas up to 150 psi. After 14 h the reactor was depressurized and filtered through a pad of Celite and washed with 10 mL of ethyl acetate. Solvent was removed and the residue was dissolved in dichloromethane (2 mL), TFA (2 mL, 25.96 mmol) and stirred for 15 min. A saturated solution of NaHCO₃ (5 mL) was added and extracted with dichloromethane (3×10 mL). Organic layers were combined, dried over Na₂SO₄, filtered, and concentrated. The residue was purified by reverse phase chromatography using a mobile gradient from 20% acetonitrile in water to 80% acetonitrile over 10 minutes provided the first eluted diastereomer pair, 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 1) (16 mg, 36%). ¹H NMR (400 MHz, acetone-d₆) δ 7.92 (s, 1H), 6.76 (s, 2H), 6.53 (s, 1H), 2.98 (m, 1H), 2.82 (s, 1H), 2.37-2.15 (m, 2H), 2.01-1.80 (m, 3H), 1.71-1.42 (m, 5H), 1.37-1.28 (m, 1H) ppm. ESI-MS m/z calc. 508.1157, found 509.1 (M+1)⁺; Retention time: 0.97 minutes (LC Method J).

The later eluting diastereomer pair provided 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 2) (15 mg, 34%). ¹H NMR (400 MHz, acetone-d₆) δ 7.93 (s, 1H), 6.78 (s, 2H), 6.44 (s, 1H), 3.16-2.87 (m, 1H), 2.80 (s, 1H), 2.54-2.10 (m, 2H), 1.94-1.79 (m, 1H), 1.72-1.59 (m, 3H), 1.54-1.44 (m, 5H) ppm. ESI-MS m/z calc. 508.1157, found 509.1 (M+1)⁺; Retention time: 1.11 minutes (LC Method J).

### Step 7: 17-Amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 1, Enantiomer 1) (Compound 25) and 17-amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 1, Enantiomer 2) (Compound 26)

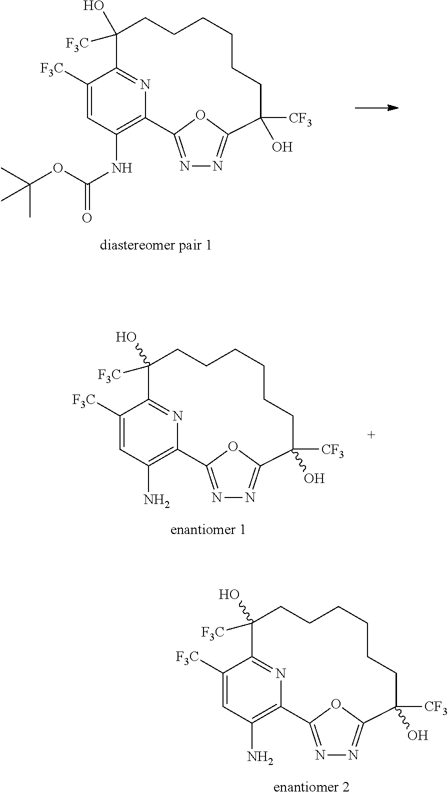 [SMILES: CC(C)(C)OC(=O)Nc1cc(C(F)(F)F)c2nc1C1=NN=C(O1)C(O)(C(F)(F)F)CCCCCCC2(O)C(F)(F)F ; Nc1cc(C(F)(F)F)c2nc1C1=NN=C(O1)C(O)(C(F)(F)F)CCCCCCC2(O)C(F)(F)F ; Nc1cc(C(F)(F)F)c2nc1C1=NN=C(O1)C(O)(C(F)(F)F)CCCCCCC2(O)C(F)(F)F]

17-Amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 1) (15 mg, 0.02951 mmol) was purified by SFC chromatography using a ChiralCel OJ-3 column (250×10 mm, 5 m particle size) with 16% methanol (20 mM NH₃) and 84% carbon dioxide mobile phase at a flow rate of 10 mL/min (injection volume=70 μL, ˜20 mg/mL solution in methanol) over 8 minutes which gave as a white solid and the first enantiomer to elute, 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 1, enantiomer 1) (5.6 mg, 74%). ¹H NMR (400 MHz, acetone-d₆) δ 7.92 (s, 1H), 6.76 (s, 2H), 6.53 (s, 1H), 2.98 (m, 1H), 2.82 (s, 1H), 2.37-2.15 (m, 2H), 2.01-1.80 (m, 3H), 1.71-1.42 (m, 5H), 1.37-1.28 (m, 1H) ppm. ESI-MS m/z calc. 508.1157, found 509.1 (M+1)⁺; Retention time: 0.97 minutes (LC Method J).

The second eluting enantiomer was isolated as a white solid, 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 1, enantiomer 2) (1.9 mg, 25%). ¹H NMR (400 MHz, acetone-d₆) δ 7.92 (s, 1H), 6.76 (s, 2H), 6.53 (s, 1H), 2.98 (m, 1H), 2.82 (s, 1H), 2.37-2.15 (m, 2H), 2.01-1.80 (m, 3H), 1.71-1.42 (m, 5H), 1.37-1.28 (m, 1H) ppm. ESI-MS m/z calc. 508.1157, found 509.1 (M+1)⁺; Retention time: 0.97 minutes (LC Method J).

### Step 8: 17-Amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 2, Enantiomer 3) (Compound 27) and 17-amino-6,13,15-tris(trifluoromethyl)-19-oxa-3,4,18-triazatricyclo[12.3.1.12,5]nonadeca-1(18),2,4,14,16-pentaene-6,13-diol (Diastereomer Pair 2, Enantiomer 4) (Compound 28)

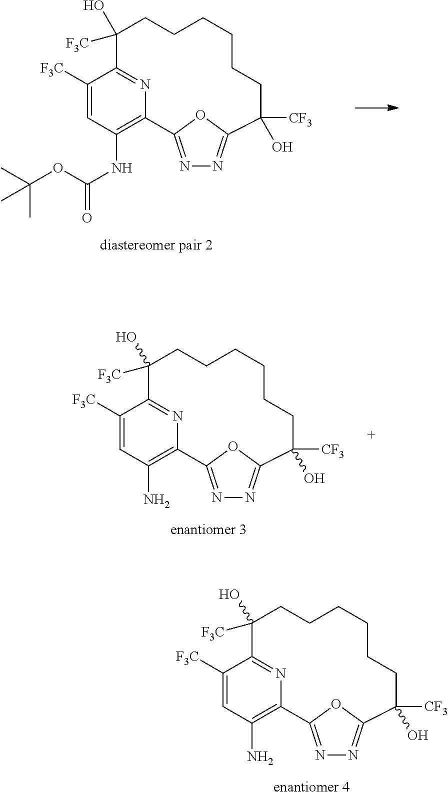 [SMILES: CC(C)(C)OC(=O)Nc1cc(C(F)(F)F)c2nc1C1=NN=C(O1)C(O)(C(F)(F)F)CCCCCCC2(O)C(F)(F)F ; Nc1cc(C(F)(F)F)c2nc1C1=NN=C(O1)C(O)(C(F)(F)F)CCCCCCC2(O)C(F)(F)F ; Nc1cc(C(F)(F)F)c2nc1C1=NN=C(O1)C(O)(C(F)(F)F)CCCCCCC2(O)C(F)(F)F]

17-Amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 2) (14 mg, 0.02754 mmol) was purified by SFC chromatography using a ChiralCel OJ-3 column (250×10 mm, 5 m particle size) with 16% methanol (20 mM NH₃) and 84% carbon dioxide mobile phase at a flow rate of 10 mL/min (injection volume=70 μL, ˜20 mg/mL solution in methanol) over 8 minutes which gave as a white solid and the first enantiomer to elute, 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 2, enantiomer 3) (6.1 mg, 85%). ¹H NMR (400 MHz, acetone-d₆) δ 7.93 (s, 1H), 6.78 (s, 2H), 6.44 (s, 1H), 3.16-2.87 (m, 1H), 2.80 (s, 1H), 2.54-2.10 (m, 2H), 1.94-1.79 (m, 1H), 1.72-1.59 (m, 3H), 1.54-1.44 (m, 5H) ppm. ESI-MS m/z calc. 508.1157, found 509.1 (M+1)⁺; Retention time: 1.11 minutes (LC Method J).

The second eluting enantiomer was isolated as a white solid, 17-amino-6,13,15-tris(trifluoromethyl)-19-oxa-3,4,18-triazatricyclo[12.3.1.12,5]nonadeca-1(18),2,4,14,16-pentaene-6,13-diol (diastereomer pair 2, enantiomer 4) (5.2 mg, 73%). ¹H NMR (400 MHz, acetone-d₆) δ 7.93 (s, 1H), 6.78 (s, 2H), 6.44 (s, 1H), 3.16-2.87 (m, 1H), 2.80 (s, 1H), 2.54-2.10 (m, 2H), 1.94-1.79 (m, 1H), 1.72-1.59 (m, 3H), 1.54-1.44 (m, 5H) ppm. ESI-MS m/z calc. 508.1157, found 509.1 (M+1)⁺; Retention time: 1.11 minutes (LC Method J).

### Example 13: Preparation of (6R)-17-amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z Isomer 1) (Hydrochloride Salt) (Compound 29) and (6R)-17-amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z Isomer 2) (Hydrochloride Salt) (Compound 30)

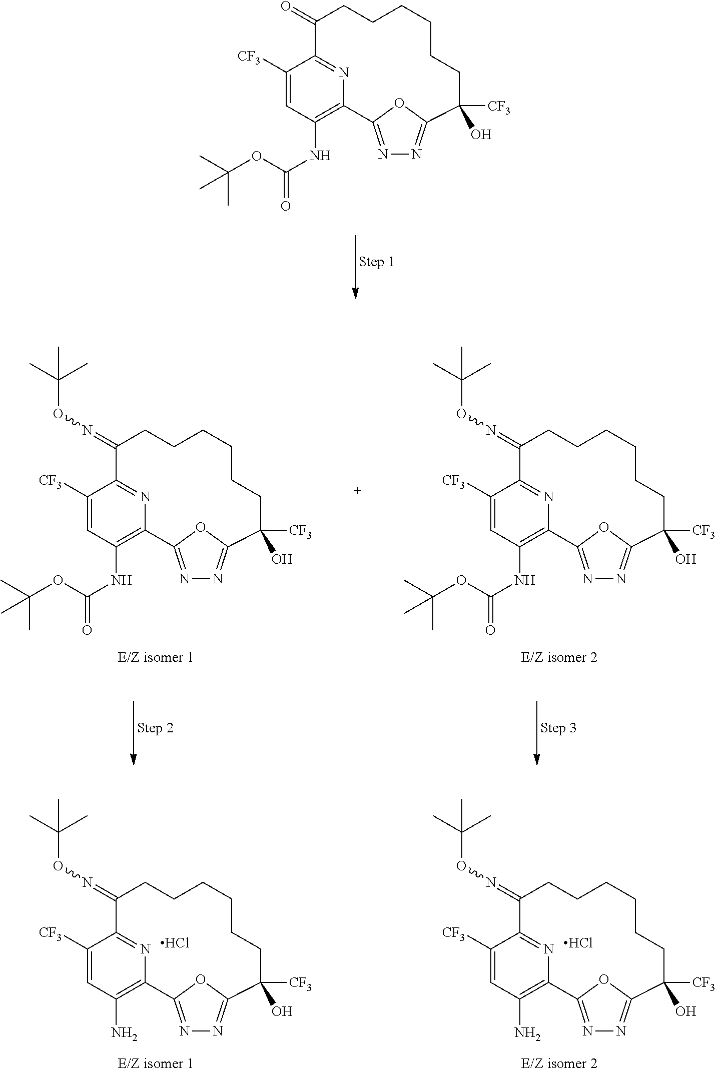

### Step 1: tert-Butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z Isomer 1) and tert-butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z Isomer 2)

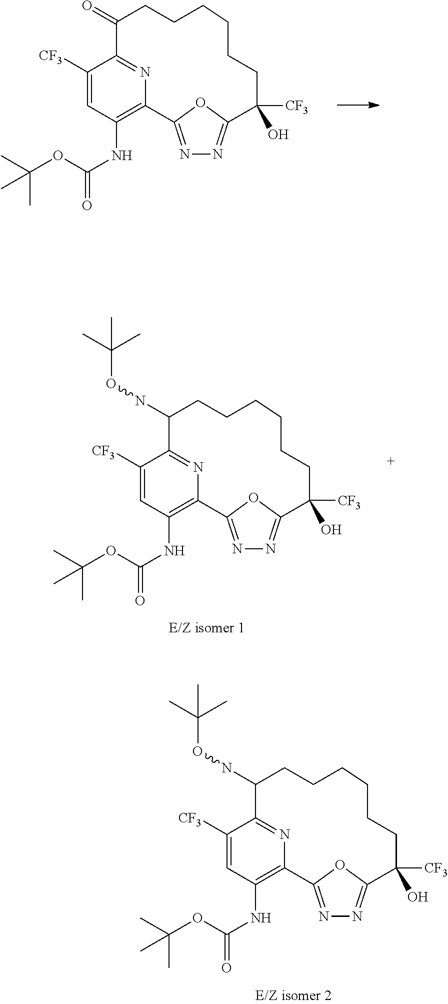 [SMILES: CC(C)(C)OC(=O)Nc1cc(C(F)(F)F)c2nc1C1=NN=C(O1)[C@@](O)(C(F)(F)F)CCCCCCC2=O ; CC(C)(C)ONC1CCCCCC[C@](O)(C(F)(F)F)C2=NN=C(O2)c2nc1c(C(F)(F)F)cc2NC(=O)OC(C)(C)C ; CC(C)(C)ONC1CCCCCC[C@](O)(C(F)(F)F)C2=NN=C(O2)c2nc1c(C(F)(F)F)cc2NC(=O)OC(C)(C)C]

To a solution of tert-butyl N-[(6R)-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (27 mg, 0.05014 mmol) in EtOH (1 mL), acetic acid (1 mL) was added O-tert-butylhydroxylamine (hydrochloride salt) (38 mg, 0.3026 mmol) followed by NaOAc (42 mg, 0.5120 mmol). The reaction mixture was heated at 80° C. for overnight. Anhydrous magnesium sulfate was added and heated the reaction at 80° C. for another 6 h, stopping the reaction at ˜60% conversion. The reaction mixture was filtered, and the filtrate was concentrated. The residue was purified by reverse phase HPLC using a gradient from 50% to 99% acetonitrile in water (+5 mM HCl) over 15 minutes giving as an off-white solid and the first isomer to elute, tert-butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z isomer 1) (8.4 mg, 55%). ESI-MS m/z calc. 609.2386, found 610.2 (M+1)⁺; Retention time: 1.76 minutes (LC Method M).

The second enantiomer to elute was isolated as an off-white solid, tert-butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z isomer 2) (6.7 mg, 44%). ESI-MS m/z calc. 609.2386, found 610.2 (M+1)⁺; Retention time: 1.96 minutes (LC Method M).

### Step 2: (6R)-17-Amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z isomer 1) (Hydrochloride Salt) (Compound 29)

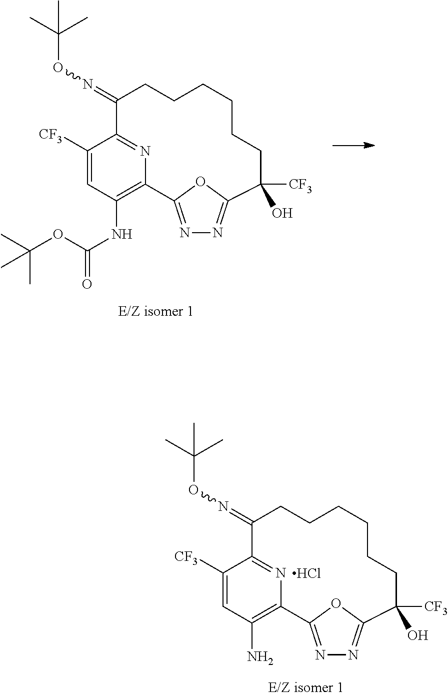 [SMILES: CC(C)(C)ON=C1CCCCCC[C@](O)(C(F)(F)F)C2=NN=C(O2)c2nc1c(C(F)(F)F)cc2NC(=O)OC(C)(C)C ; CC(C)(C)ON=C1CCCCCC[C@](O)(C(F)(F)F)C2=NN=C(O2)c2nc1c(C(F)(F)F)cc2N ; Cl]

tert-Butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z isomer 1) (8.4 mg, 0.01378 mmol) was dissolved in (pre made solution of 1:4 TFA/dichloromethane) TFA (100 μL, 1.298 mmol) and dichloromethane (400 μL) and the reaction was stirred at room temperature for about 1 h. Solvents were removed and the residue was purified by reverse phase HPLC using a gradient from 30% to 99% acetonitrile in water (+5 mM HCl) over 15 minutes which gave as a white solid, (6R)-17-amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z isomer 1) (hydrochloride salt) (3.9 mg, 52%). ¹H NMR (500 MHz, DMSO-d₆) δ 7.71 (s, 1H), 7.65 (s, 1H), 6.90 (s, 2H), 2.45 (s, 2H), 2.17 (s, 2H), 1.67 (s, 2H), 1.57 (s, 2H), 1.41 (s, 4H), 1.16 (s, 9H) ppm. ESI-MS m/z calc. 509.18616, found 510.1 (M+1)⁺; Retention time: 1.71 minutes (LC Method J).

### Step 3: (6R)-17-Amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z Isomer 2) (Hydrochloride Salt) (Compound 30)

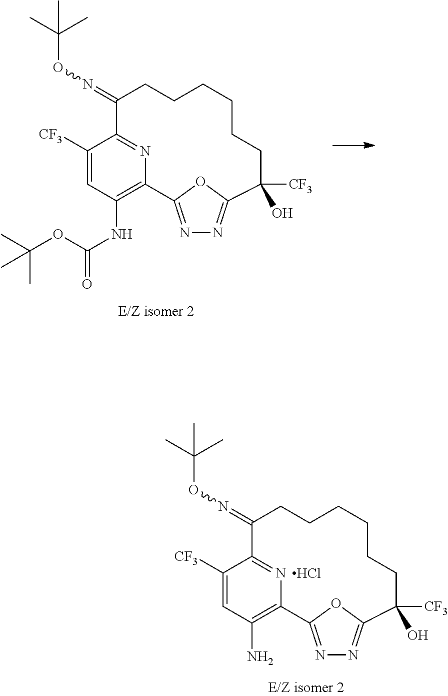 [SMILES: CC(C)(C)ON=C1CCCCCC[C@](O)(C(F)(F)F)C2=NN=C(O2)c2nc1c(C(F)(F)F)cc2NC(=O)OC(C)(C)C ; CC(C)(C)ON=C1CCCCCC[C@](O)(C(F)(F)F)C2=NN=C(O2)c2nc1c(C(F)(F)F)cc2N ; Cl]

tert-Butyl N-[(6R)-13-tert-butoxyimino-6-hydroxy-6,15-bis(trifluoromethyl)-19-oxa-3,4,18-triazatricyclo[12.3.1.12,5]nonadeca-1(17),2,4,14(18),15-pentaen-17-yl]carbamate (E/Z isomer 2) (6.7 mg, 0.01099 mmol) was dissolved in a (pre made solution of 1:4 TFA/dichloromethane) TFA (100 μL, 1.298 mmol) and dichloromethane (400 μL) and the reaction was stirred at room temperature for about 1 h. Solvents were removed and the residue was purified by reverse phase HPLC using a gradient from 30% to 99% acetonitrile in water (+5 mM HCl) over 15 minutes which gave as a white solid, (6R)-17-amino-13-tert-butoxyimino-6,15-bis(trifluoromethyl)-19-oxa-3,4,18-triazatricyclo[12.3.1.12,5]nonadeca-1(18),2,4,14,16-pentaen-6-ol (E/Z isomer 2) (hydrochloride salt) (2 mg, 27%). ESI-MS m/z calc. 509.18616, found 510.1 (M+1)⁺; Retention time: 1.93 minutes (LC Method M).

### Example 14: Preparation of (6R)-17-amino-6-hydroxy-6,15-bis(trifluoromethyl)-19-oxa-3,4,18-triazatricyclo[12.3.1.12,5]nonadeca-1(18),2,4,14,16-pentaen-13-one oxime (Cis or Trans Oxime) (Compound 31)

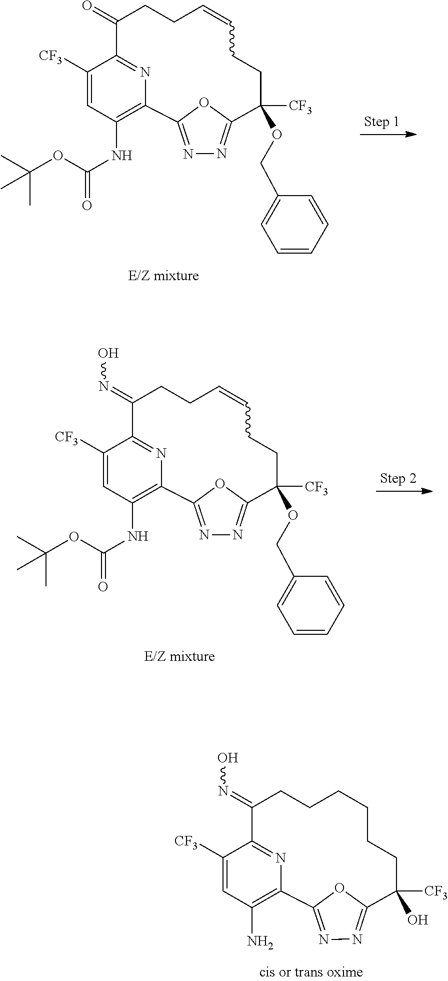 [SMILES: CC(C)(C)OC(=O)Nc1cc(C(F)(F)F)c2nc1C1=NN=C(O1)[C@@](OCc1ccccc1)(C(F)(F)F)CCC=CCCC2=O ; CC(C)(C)OC(=O)Nc1cc(C(F)(F)F)c2nc1C1=NN=C(O1)[C@@](OCc1ccccc1)(C(F)(F)F)CCC=CCCC2=NO ; Nc1cc(C(F)(F)F)c2nc1C1=NN=C(O1)[C@@](O)(C(F)(F)F)CCCCCCC2=NO]

### Step 1: tert-Butyl N-[(6R)-6-benzyloxy-13-hydroxyimino-6,15-bis(trifluoromethyl)-19-oxa-3,4,18-triazatricyclo[12.3.1.12,5]nonadeca-1(17),2,4,9,14(18),15-hexaen-17-yl]carbamate (E/Z Mixture)

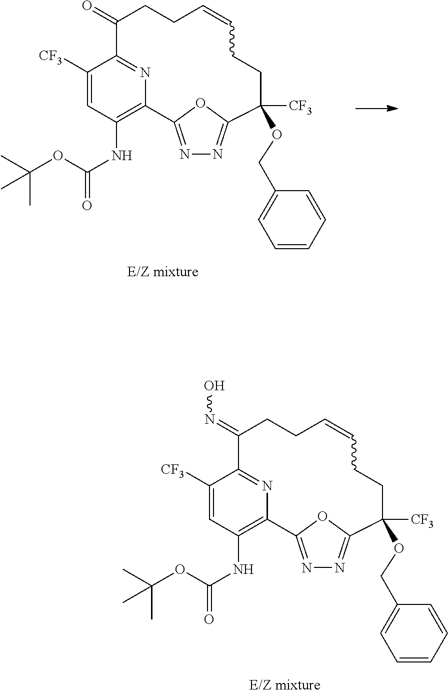 [SMILES: CC(C)(C)OC(=O)Nc1cc(C(F)(F)F)c2nc1C1=NN=C(O1)[C@@](OCc1ccccc1)(C(F)(F)F)CCC=CCCC2=O ; CC(C)(C)OC(=O)Nc1cc(C(F)(F)F)c2nc1C1=NN=C(O1)[C@@](OCc1ccccc1)(C(F)(F)F)CCC=CCCC2=NO]

To a solution of tert-butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (150 mg, 0.2394 mmol) in EtOH (3 mL) was added hydroxylamine (hydrochloride salt) (50 mg, 0.7195 mmol) followed by NaOAc (98 mg, 1.195 mmol). The reaction mixture was heated at 80° C. for overnight. Reaction was filtered and filtrate was concentrated, then purified by silica gel chromatography using a gradient from 0% to 30% ethyl acetate in hexanes which gave as a white solid, tert-butyl N-[(6R)-6-benzyloxy-13-hydroxyimino-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (125 mg, 81%). ESI-MS m/z calc. 641.2073, found 462.2 (M+1)⁺; Retention time: 0.95 minutes (LC Method T).

### Step 2: (6R)-17-Amino-6-hydroxy-6,15-bis(trifluoromethyl)-19-oxa-3,4,18-triazatricyclo[12.3.1.12,5]nonadeca-1(18),2,4,14,16-pentaen-13-one oxime (Cis or Trans Oxime) (Compound 31)

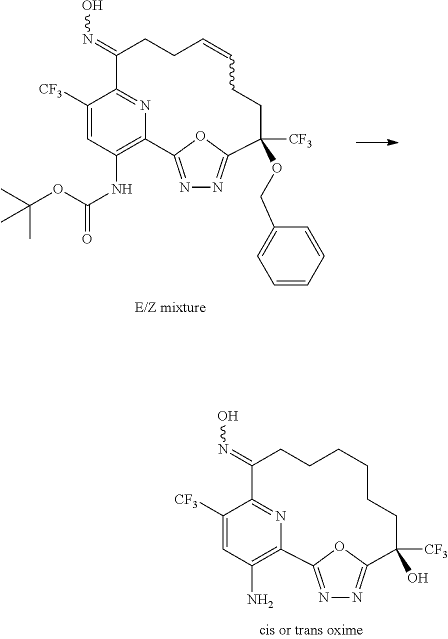 [SMILES: CC(C)(C)OC(=O)Nc1cc(C(F)(F)F)c2nc1C1=NN=C(O1)[C@@](OCc1ccccc1)(C(F)(F)F)CCC=CCCC2=NO ; Nc1cc(C(F)(F)F)c2nc1C1=NN=C(O1)[C@@](O)(C(F)(F)F)CCCCCCC2=NO]

Part 1: To a solution of tert-butyl N-[(6R)-6-benzyloxy-13-hydroxyimino-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (30 mg, 0.04676 mmol) in MeOH (450 μL) was added Pd/C (20 mg of 10% w/w, 0.01879 mmol). The mixture was stirred overnight under a hydrogen atmosphere, 200 psi. The reaction mixture was filtered through Celite, washing well with ethyl acetate and then concentrated to give 25 mg of a yellow residue which was used directly in part 2.

Part 2: To a solution of the yellow residue obtained in part 1 (20 mg) in TFA (250 μL) was stirred at room temperature for 5 minutes. Reaction mixture was filtered through Celite, washing well with ethyl acetate and then concentrated. Diluted with MeOH and purified by reverse phase chromatography using 30% to 99% acetonitrile and water (+0.05 mM HCl) which gave as a white solid, (6R)-17-amino-6-hydroxy-6,15-bis(trifluoromethyl)-19-oxa-3,4,18-triazatricyclo[12.3.1.12,5]nonadeca-1(18),2,4,14,16-pentaen-13-one oxime (cis or trans oxime) (5 mg, 30%, 2 steps). ¹H NMR (400 MHz, methanol-d4) δ 7.66 (s, 1H), 2.82 (m, 1H), 2.61 (m, 1H), 2.23 (t, J=7.8 Hz, 2H), 2.12-2.02 (m, 1H), 1.79 (m, 3H), 1.61 (m, 4H) ppm. ESI-MS m/z calc. 453.12357, found 454.17 (M+1)⁺; Retention time: 1.64 minutes (LC Method A).

### Example 15: Preparation of 20-amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (Enantiomer 1) (Compound 32) and 20-amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (Enantiomer 2) (Compound 33)

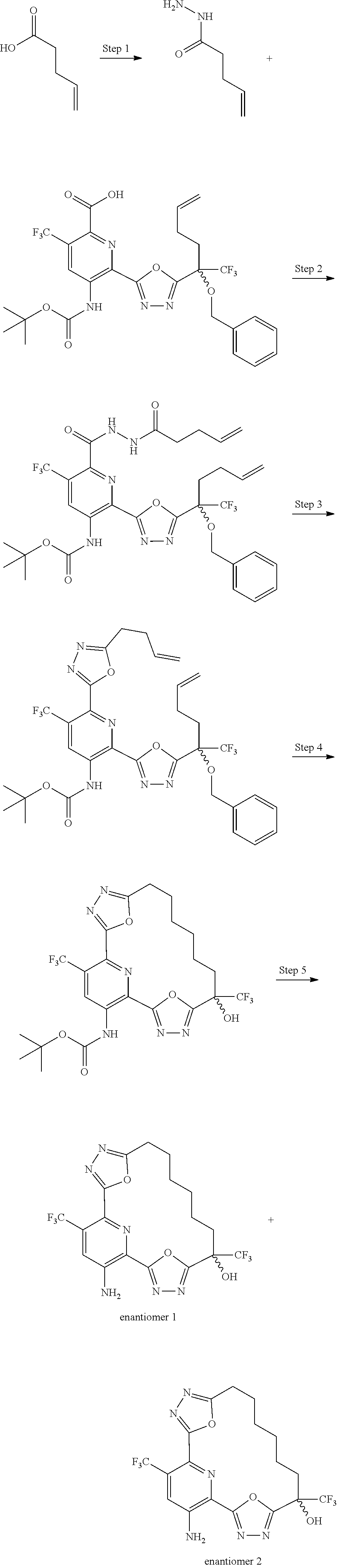

### Step 1: Pent-4-enehydrazide

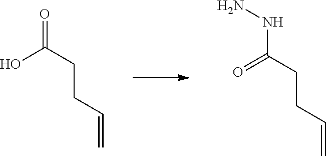

To a stirred solution of pent-4-enoic acid (1 g, 9.988 mmol) and tert-butyl N-aminocarbamate (6.6 g, 49.94 mmol) in ethyl acetate (45 mL) was added T3P (31.8 g of 50% w/w, 49.97 mmol), followed by addition of pyridine (15.8 g, 199.7 mmol). The reaction mixture was stirred at room temperature for 16 h. Then, saturated solution of sodium bicarbonate was slowly added till no bubble formation was observed. The organic compound was extracted with ethyl acetate (3×10 mL), combined organics dried over sodium sulfate, filtered and concentrated. Purification by silica gel chromatography using 75:25 hexanes and ethyl acetate gave a pale-yellow oil. Next, the compound was dissolved in dichloromethane (15 mL) and cooled in an ice bath and TFA (10 mL, 129.8 mmol) was added slowly and let the reaction mixture warm to room temperature and stirred for an additional 1 h. After this time, saturated solution of sodium bicarbonate was added to the mixture (pH=7). The compound was extracted with ethyl acetate (4×15 mL). Organic layers were combined, dried over sodium sulfate, filtered, and concentrated under vacuum to afford pent-4-enehydrazide (418 mg, 37%). ¹H NMR (400 MHz, CDCl₃) δ 7.39 (bs, 1H), 5.86-5.75 (m, 1H), 5.39-4.80 (m, 2H), 3.95 (bs, 2H), 2.58-2.35 (m, 2H), 2.33-2.03 (m, 2H) ppm.

### Step 2: tert-Butyl N-[2-[5-[1-benzyloxy-1-(trifluoromethyl)pent-4-enyl]-1,3,4-oxadiazol-2-yl]-6-[(pent-4-enoylamino)carbamoyl]-5-(trifluoromethyl)-3-pyridyl]carbamate

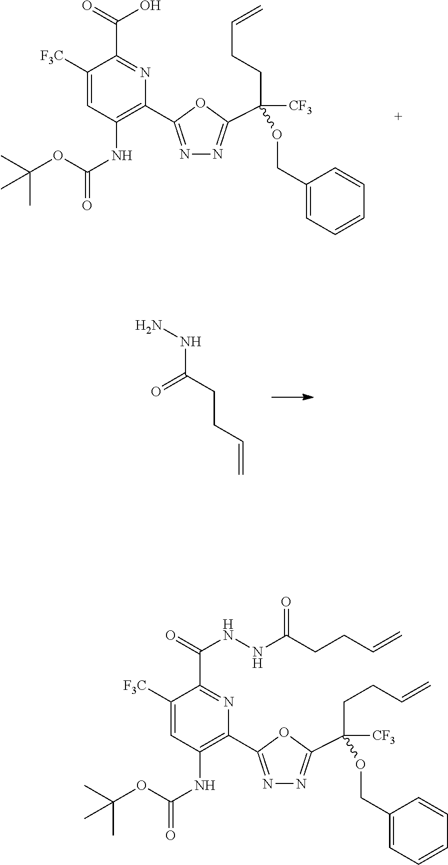

To a solution of 6-[5-[1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic acid (205 mg, 0.3325 mmol) and pent-4-enehydrazide (45.6 mg, 0.3995 mmol) in NMP (10 mL) was added DIEA (129 mg, 0.9981 mmol), followed by addition of HATU (190 mg, 0.4997 mmol). The reaction mixture was stirred at room temperature for 35 minutes. The mixture was diluted with water (10 mL) and extracted with ethyl acetate (3×10 mL). The organic phases combined and dried over Na₂SO₄, filtered, and concentrated in vacuo. The residue was purified by reverse phase chromatography using a gradient from 30% to 95% acetonitrile in water (+5 mM HCl) over 10 minutes giving tert-butyl N-[2-[5-[1-benzyloxy-1-(trifluoromethyl)pent-4-enyl]-1,3,4-oxadiazol-2-yl]-6-[(pent-4-enoylamino)carbamoyl]-5-(trifluoromethyl)-3-pyridyl]carbamate (122 mg, 51%). ESI-MS m/z calc. 712.2444, found 713.3 (M+1)⁺; Retention time: 1.31 minutes (LC Method M).

### Step 3: tert-Butyl N-[2-[5-[1-benzyloxy-1-(trifluoromethyl)pent-4-enyl]-1,3,4-oxadiazol-2-yl]-6-(5-but-3-enyl-1,3,4-oxadiazol-2-yl)-5-(trifluoromethyl)-3-pyridyl]carbamate

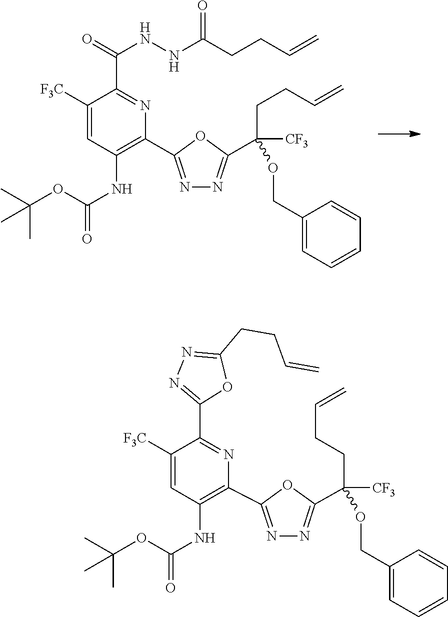 [SMILES: C=CCCC(=O)NNC(=O)c1nc(-c2nnc(C(CCC=C)(OCc3ccccc3)C(F)(F)F)o2)c(NC(=O)OC(C)(C)C)cc1C(F)(F)F ; C=CCCc1nnc(-c2nc(-c3nnc(C(CCC=C)(OCc4ccccc4)C(F)(F)F)o3)c(NC(=O)OC(C)(C)C)cc2C(F)(F)F)o1]

A solution of tert-butyl N-[2-[5-[1-benzyloxy-1-(trifluoromethyl)pent-4-enyl]-1,3,4-oxadiazol-2-yl]-6-[(pent-4-enoylamino)carbamoyl]-5-(trifluoromethyl)-3-pyridyl]carbamate (130 mg, 0.1824 mmol) and DIEA (59 mg, 0.4565 mmol) in acetonitrile (5 mL) was heated to 50° C., then 4-methylbenzenesulfonyl chloride (52.2 mg, 0.2738 mmol) was added. The resulted mixture was heated at 70° C. for 100 minutes, the reaction mixture was cooled, and quenched with saturated solution of sodium bicarbonate (10 mL) and extracted with ethyl acetate (3×10 mL). Combined organic layers washed with brine (30 mL), dried over sodium sulfate, filtered and evaporated. Purification by silica gel chromatography using 80:20 hexanes and ethyl acetate provided tert-butyl N-[2-[5-[1-benzyloxy-1-(trifluoromethyl)pent-4-enyl]-1,3,4-oxadiazol-2-yl]-6-(5-but-3-enyl-1,3,4-oxadiazol-2-yl)-5-(trifluoromethyl)-3-pyridyl]carbamate (86 mg, 68%). ¹H NMR (400 MHz, CDCl₃) δ 10.45 (s, 1H), 9.53 (s, 1H), 7.53-7.41 (m, 2H), 7.38-7.27 (m, 3H), 5.88 (m, 1H), 5.76 (m, 1H), 5.20-4.92 (m, 4H), 4.84 (d, J=10.6 Hz, 1H), 4.70 (d, J=10.5 Hz, 1H), 3.08 (t, J=7.5 Hz, 2H), 2.72-2.57 (m, 2H), 2.56-2.45 (m, 2H), 2.42-2.31 (m, 1H), 2.26-2.17 (m, 1H), 1.59 (s, 9H) ppm. ESI-MS m/z calc. 694.2338, found 695.0 (M+1)⁺; Retention time: 1.69 minutes (LC Method M).

### Step 4: tert-Butyl N-[12-hydroxy-12,20-bis(trifluoromethyl)-22,23-dioxa-3,4,14,15,21-pentaazatetracyclo[15.3.1.12,5.113,16]tricosa-1(20),2,4,13,15,17(21),18-heptaen-18-yl]carbamate

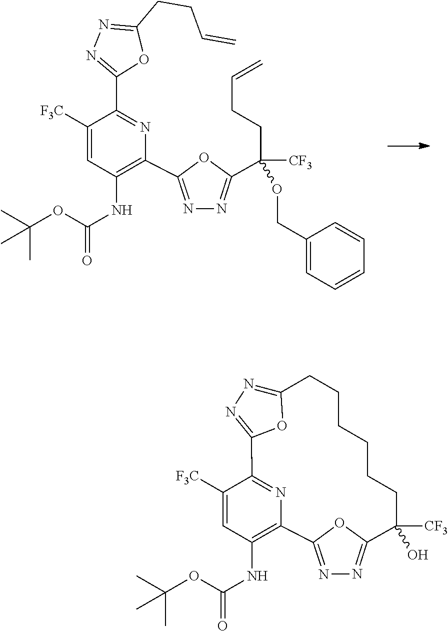 [SMILES: C=CCCc1nnc(-c2nc(-c3nnc(C(CCC=C)(OCc4ccccc4)C(F)(F)F)o3)c(NC(=O)OC(C)(C)C)cc2C(F)(F)F)o1 ; CC(C)(C)OC(=O)Nc1cc(C(F)(F)F)c2nc1C1=NN=C(O1)C(O)(C(F)(F)F)CCCCCCC1=NN=C2O1]

A stirred solution of tert-butyl N-[2-[5-[1-benzyloxy-1-(trifluoromethyl)pent-4-enyl]-1,3,4-oxadiazol-2-yl]-6-(5-but-3-enyl-1,3,4-oxadiazol-2-yl)-5-(trifluoromethyl)-3-pyridyl]carbamate (98 mg, 0.1411 mmol) in 1,2-dichloroethane (25 mL) was heated up to 70° C. and dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (15.5 mg, 0.02112 mmol) was added. The reaction mixture was heated at 85° C. for 1.5 h, cooled to room temperature and solvent was evaporated. Purification by silica gel chromatography using 15:85 ethyl acetate and hexanes provided the desired macrocyclization compound. The residue was dissolved in acetic acid (3 mL), transferred to a high-pressure reactor and Pd/C (20 mg, 10% w/w, 0.009397 mmol, 50% wet) was added. The reactor was evacuated and filled with nitrogen gas three times, finally evacuated and filled with H₂ up to 60 psi. The mixture was stirred for 6 hours and then, depressurized. Filtered through a pad of Celite, washed with ethyl acetate and solvents were evaporated. Purification by reverse phase chromatography using a gradient from 20% to 90% acetonitrile in water (+5 mM HCl) over 12 minutes gave tert-butyl N-[12-hydroxy-12,20-bis(trifluoromethyl)-22,23-dioxa-3,4,14,15,21-pentaazatetracyclo[15.3.1.12,5.113,16]tricosa-1(20),2,4,13,15,17(21),18-heptaen-18-yl]carbamate (30 mg, 37%). ¹H NMR (400 MHz, CDCl₃) δ 9.70 (s, 1H), 9.45 (s, 1H), 4.04 (bs, 1H), 3.21 (m, 1H), 2.98 (m, 1H), 2.46 (m, 1H), 2.28 (m, 1H), 2.13-1.69 (m, 6H), 1.59 (s, 9H), 153-1.39 (m, 2H) ppm. ESI-MS m/z calc. 578.17126, found 579.1 (M+1)⁺; Retention time: 1.55 minutes (LC Method J).

### Step 5: 20-Amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (Enantiomer 1) (Compound 32) and 20-amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (Enantiomer 2) (Compound 33)

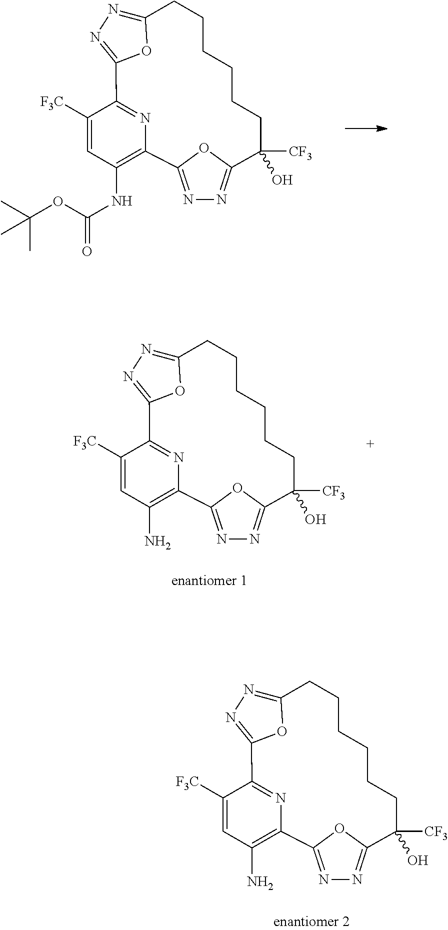 [SMILES: CC(C)(C)OC(=O)Nc1cc(C(F)(F)F)c2nc1C1=NN=C(O1)C(O)(C(F)(F)F)CCCCCCC1=NN=C2O1 ; Nc1cc(C(F)(F)F)c2nc1C1=NN=C(O1)C(O)(C(F)(F)F)CCCCCCC1=NN=C2O1 ; Nc1cc(C(F)(F)F)c2nc1C1=NN=C(O1)C(O)(C(F)(F)F)CCCCCCC1=NN=C2O1]

To a stirred solution of tert-butyl N-[12-hydroxy-12,20-bis(trifluoromethyl)-22,23-dioxa-3,4,14,15,21-pentaazatetracyclo[15.3.1.12,5.113,16]tricosa-1(20),2,4,13,15,17(21),18-heptaen-18-yl]carbamate (30 mg, 0.05186 mmol) in dichloromethane (1.5 mL) was added TFA (1.5 mL, 19.47 mmol) at room temperature and the mixture was stirred for 35 minutes. Solvent was evaporated under reduced pressure. Purification by chiral SFC using a ChiralCel OZ-3 column (250×10 mm, 5 m particle size), and a dual gradient run with 18% methanol (20 mM NH₃) 82% carbon dioxide mobile phase at a flow rate of 10 mL/min over 10 minutes giving as the first enantiomer to elute 20-amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (enantiomer 1) (10 mg, 40%). ¹H NMR (400 MHz, acetone-d₆) δ 7.96 (s, 1H), 7.16 (s, 2H), 6.54 (s, 1H), 3.21-3.07 (m, 1H), 3.04-2.96 (m, 1H), 2.42 (m, 1H), 2.29 (m, 1H), 2.02-1.90 (m, 1H), 1.90-1.73 (m, 4H), 1.71-1.48 (m, 3H) ppm. ESI-MS m/z calc. 478.1188, found 479.0 (M+1)⁺; Retention time: 0.8 minutes (LC Method J).

The later eluting enantiomer provided, 20-amino-6,18-bis(trifluoromethyl)-22,23-dioxa-3,4,14,15,21-pentaazatetracyclo[15.3.1.12,5.113,16]tricosa-1(21),2,4,13,15,17,19-heptaen-6-ol (enantiomer 2) (10 mg, 40%). ¹H NMR (400 MHz, acetone-d₆) δ 7.96 (s, 1H), 7.16 (s, 2H), 6.54 (s, 1H), 3.21-3.07 (m, 1H), 3.04-2.96 (m, 1H), 2.42 (m, 1H), 2.29 (m, J=14.1, 11.8, 5.3 Hz, 1H), 2.02-1.90 (m, 1H), 1.90-1.73 (m, 4H), 1.71-1.48 (m, 3H) ppm. ESI-MS m/z calc. 478.1188, found 479.0 (M+1)⁺; Retention time: 0.8 minutes (LC Method J).

### Example 16: Preparation of 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (enantiomer 1) (Compound 34) and 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (Enantiomer 2) (Compound 35)

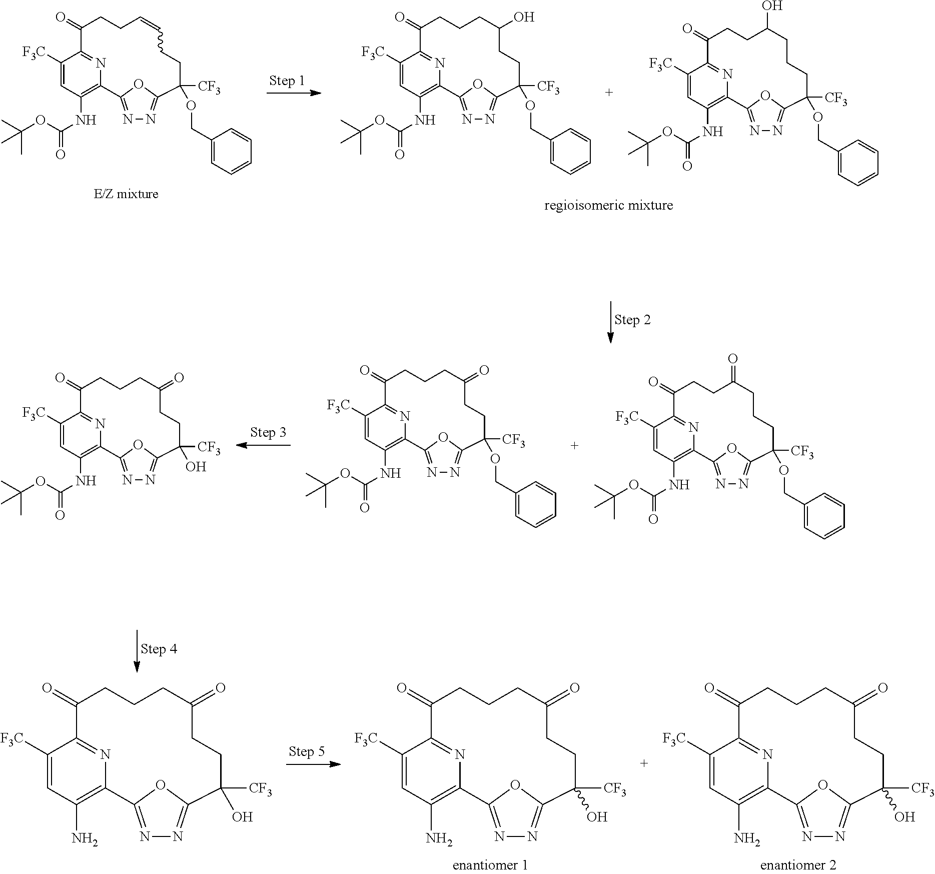

### Step 1: tert-Butyl N-[6-benzyloxy-9-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate and tert-butyl N-[6-benzyloxy-10-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (Regioisomeric Mixture)

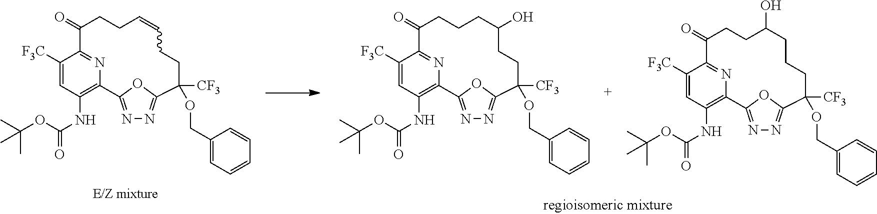 [SMILES: CC(C)(C)OC(=O)Nc1cc(C(F)(F)F)c2nc1C1=NN=C(O1)C(OCc1ccccc1)(C(F)(F)F)CCC=CCCC2=O ; CC(C)(C)OC(=O)Nc1cc(C(F)(F)F)c2nc1C1=NN=C(O1)C(OCc1ccccc1)(C(F)(F)F)CCC(O)CCCC2=O ; CC(C)(C)OC(=O)Nc1cc(C(F)(F)F)c2nc1C1=NN=C(O1)C(OCc1ccccc1)(C(F)(F)F)CCCC(O)CCC2=O]

To a solution of tert-butyl N-[6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (850 mg, 1.316 mmol) in THF (15 mL) at 0° C. was dropwise added borane dimethyl sulfide complex (995 μL of 2 M, 1.99 mmol) and stirred for 15 min at 0° C. Allowed the reaction to warm to room temperature and stirred for 1 h. Cooled the reaction to 0° C. before quenching with aqueous NaOH (6 mL of 1 M, 6 mmol) followed by the addition of hydrogen peroxide (7 mL of 30% w/v, 61.74 mmol). This mixture was stirred for 30 min at room temperature then was extracted with ethyl acetate (2×80 mL). The organic layers were combined, washed with brine (80 mL), dried over sodium sulfate, filtered and concentrated. The residue was purified by silica gel chromatography (80 gram column) using a gradient from 100% hexanes to 80% ethyl acetate in hexanes (product elutes at 30% to 40% ethyl acetate) to afford an unseparated mixture of two regioisomeric products as a white solid consisting of tert-butyl N-[6-benzyloxy-9-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (425 mg, 50%), ESI-MS m/z calc. 644.207, found 645.2 (M+1)⁺; Retention time: 1.73 minutes (LC Method J) and tert-butyl N-[6-benzyloxy-10-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (325 mg, 38%). ESI-MS m/z calc. 644.207, found 645.2 (M+1)⁺; Retention time: 1.73 minutes (LC Method J) which were taken directly to the ensuing step. Yields are estimated based on ratios of the two product peaks from HPLC.

### Step 2: tert-Butyl N-[6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate and tert-butyl N-[6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate

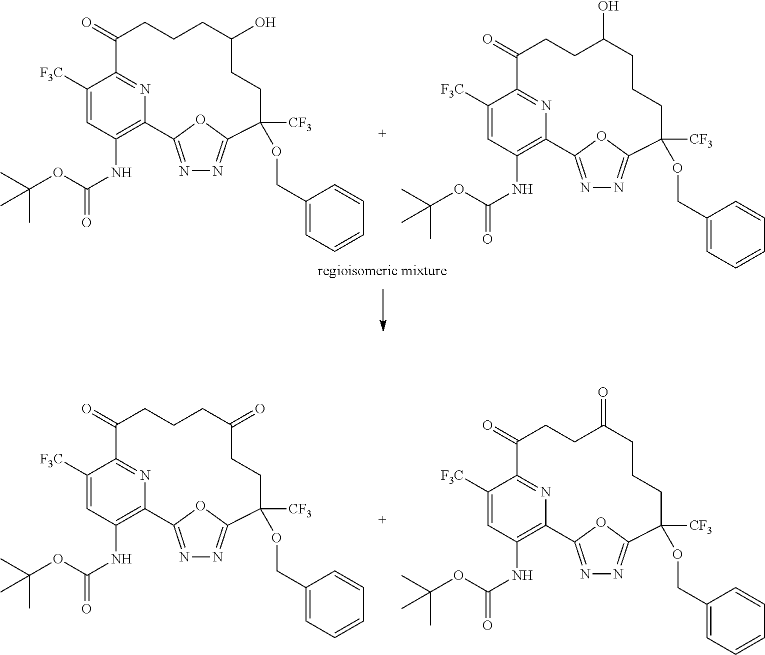

To a solution containing a regioisomeric mixture of tert-butyl N-[6-benzyloxy-9-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (425 mg, 0.6594 mmol) and tert-butyl N-[6-benzyloxy-10-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (325 mg, 0.5042 mmol) in dichloromethane (22 mL) at room temperature was added Dess-Martin periodinane (500 mg, 1.179 mmol). After 15 min, added additional Dess-Martin periodinane (500 mg, 1.179 mmol) and stirred an additional 30 minutes. Quenched the reaction with saturated aqueous sodium bicarbonate then extracted with dichloromethane (50 mL). The organic layer was washed with brine, dried over sodium sulfate, filtered and concentrated. The crude material obtained was then purified by silica gel chromatography (40 gram column) using a gradient from 100% hexanes to 70% ethyl acetate in hexanes (product elutes at 30% ethyl acetate) to afford as the first regioisomer to elute, tert-butyl N-[6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (290 mg, 69%). ESI-MS m/z calc. 642.1913, found 643.2 (M+1)⁺; Retention time: 1.91 minutes. ¹H NMR (400 MHz, DMSO-d₆) δ 9.97 (s, 1H), 9.10 (s, 1H), 7.35 (d, J=4.3 Hz, 4H), 7.29 (m, 1H), 4.72 (d, J=10.8 Hz, 1H), 4.64 (d, J=10.8 Hz, 1H), 3.29-3.13 (m, 2H), 2.89 (m, 1H), 2.78-2.69 (m, 2H), 2.69-2.58 (m, 3H), 2.10-2.00 (m, 1H), 1.99-1.88 (m, 1H), 1.53 (s, 9H) ppm. ESI-MS m/z calc. 642.1913, found 643.2 (M+1)⁺; Retention time: 1.91 minutes (LC Method J).

The later eluting and polar regioisomer gave tert-butyl N-[6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (114 mg, 35%). ¹H NMR (400 MHz, DMSO-d₆) δ 9.93 (s, 1H), 9.14 (s, 1H), 7.38-7.27 (m, 5H), 4.79-4.68 (m, 2H), 3.25 (t, J=7.4 Hz, 2H), 3.09-2.95 (m, 2H), 2.59 (m, 2H), 2.46 (d, J=4.8 Hz, 1H), 2.26 (m, 1H), 2.05-1.85 (m, 2H), 1.54 (s, 9H) ppm. ESI-MS m/z calc. 642.1913, found 643.2 (M+1)⁺; Retention time: 1.86 minutes (LC Method J).

### Step 3: tert-Butyl N-[6-hydroxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate

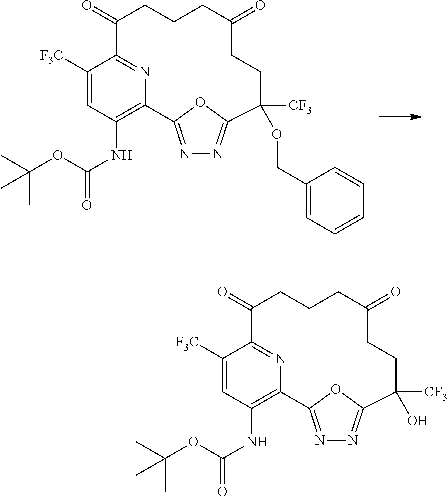 [SMILES: CC(C)(C)OC(=O)Nc1cc(C(F)(F)F)c2nc1C1=NN=C(O1)C(OCc1ccccc1)(C(F)(F)F)CCC(=O)CCCC2=O ; CC(C)(C)OC(=O)Nc1cc(C(F)(F)F)c2nc1C1=NN=C(O1)C(O)(C(F)(F)F)CCC(=O)CCCC2=O]

In a 250 mL round bottom flask, a solution of tert-butyl N-[6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (236 mg, 0.3673 mmol) in ethyl acetate (12 mL) and acetic acid (3 mL) was purged with nitrogen. Then, Pd/C (118 mg of 10% w/w, 0.1109 mmol) was added. The mixture was degassed with nitrogen then purged by a balloon filled with hydrogen gas. The mixture was stirred at 1 atm of hydrogen gas (outfitted the flask with a balloon of hydrogen) for 5 h. The reaction was purged with nitrogen then filtered and the material was diluted with dichloromethane and hexanes then concentrated. The residue was purified by silica gel chromatography (40 gram column) using a gradient from 100% hexanes to 90% ethyl acetate in hexanes (product elutes at 30% ethyl acetate) to afford as a white solid, tert-butyl N-[6-hydroxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (130 mg, 64%). ESI-MS m/z calc. 552.14435, found 553.2 (M+1)⁺; Retention time: 1.99 minutes. ¹H NMR (400 MHz, DMSO-d₆) δ 9.92 (s, 1H), 9.08 (s, 1H), 7.86 (s, 1H), 3.22 (m, 1H), 3.10 (m, 1H), 2.83-2.66 (m, 3H), 2.61 (m, 2H), 2.35 (m, 1H), 2.00 (s, 1H), 1.95-1.87 (m, 1H), 1.53 (s, 9H) ppm. ESI-MS m/z calc. 552.14435, found 553.2 (M+1)⁺; Retention time: 1.99 minutes (LC Method A).

### Step 4: 17-Amino-6-hydroxy-6,15-bis(trifluoromethyl)-19-oxa-3,4,18-triazatricyclo[12.3.1.12,5]nonadeca-1(18),2,4,14,16-pentaene-9,13-dione

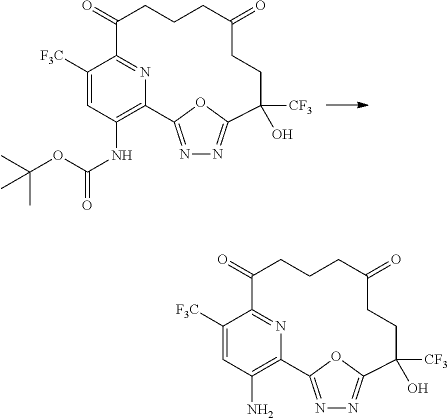 [SMILES: CC(C)(C)OC(=O)Nc1cc(C(F)(F)F)c2nc1C1=NN=C(O1)C(O)(C(F)(F)F)CCC(=O)CCCC2=O ; Nc1cc(C(F)(F)F)c2nc1C1=NN=C(O1)C(O)(C(F)(F)F)CCC(=O)CCCC2=O]

tert-Butyl N-[6-hydroxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (130 mg, 0.2353 mmol) was dissolved in dichloromethane (3 mL) and to the mixture was added TFA (750 μL, 9.735 mmol) and stirred at room temperature. After 90 mins, the mixture was evaporated to dryness, then diluted with ether and concentrated again. The crude material was then placed under vacuum for 2 h to afford as a white solid, 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (105 mg, 99%). ESI-MS m/z calc. 452.09192, found 453.2 (M+1)⁺; Retention time: 1.41 minutes (LC Method A).

### Step 5: 17-Amino-6-hydroxy-6,15-bis(trifluoromethyl)-19-oxa-3,4,18-triazatricyclo[12.3.1.12,5]nonadeca-1(18),2,4,14,16-pentaene-9,13-dione (enantiomer 1) (Compound 34) and 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (Enantiomer 2) (Compound 35)

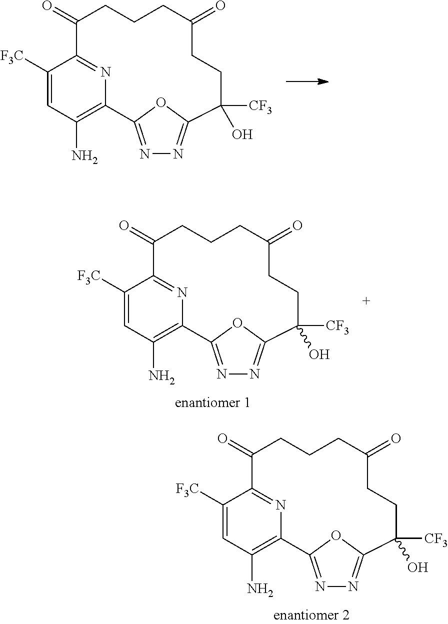 [SMILES: Nc1cc(C(F)(F)F)c2nc1C1=NN=C(O1)C(O)(C(F)(F)F)CCC(=O)CCCC2=O ; Nc1cc(C(F)(F)F)c2nc1C1=NN=C(O1)C(O)(C(F)(F)F)CCC(=O)CCCC2=O ; Nc1cc(C(F)(F)F)c2nc1C1=NN=C(O1)C(O)(C(F)(F)F)CCC(=O)CCCC2=O]

17-Amino-6-hydroxy-6,15-bis(trifluoromethyl)-19-oxa-3,4,18-triazatricyclo[12.3.1.12,5]nonadeca-1(18),2,4,14,16-pentaene-9,13-dione was purified by chiral SFC using a ChiralPak AD column (250×21.2 mm, 5 m particle size) with 5% to 80% methanol (20 mM NH₃) and carbon dioxide mobile phase which gave as a white solid and the first enantiomer to elute, 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (enantiomer 1) (46.9 mg, 88%). ¹H NMR (400 MHz, DMSO-d₆) δ 7.79 (d, J=10.8 Hz, 2H), 7.60 (s, 2H), 3.19-3.07 (m, 2H), 2.86-2.67 (m, 2H), 2.67-2.52 (m, 3H), 2.36 (m, 1H), 2.06-1.87 (m, 2H) ppm. ESI-MS m/z calc. 452.09192, found 453.2 (M+1)⁺; Retention time: 1.41 minutes (LC Method A).

The later eluting enantiomer was isolated as a white solid, 17-amino-6-hydroxy-6,15-bis(trifluoromethyl)-19-oxa-3,4,18-triazatricyclo[12.3.1.12,5]nonadeca-1(18),2,4,14,16-pentaene-9,13-dione (enantiomer 2) (46 mg, 87%). ¹H NMR (400 MHz, DMSO-d₆) δ 7.79 (d, J=12.9 Hz, 2H), 7.59 (s, 2H), 3.18-3.06 (m, 2H), 2.79 (m, 1H), 2.74-2.67 (m, 1H), 2.67-2.56 (m, 2H), 2.56-2.52 (m, 1H), 2.36 (m, 1H), 2.03-1.88 (m, 2H) ppm. ESI-MS m/z calc. 452.09192, found 453.2 (M+1)⁺; Retention time: 1.41 minutes (LC Method A).

### Example 17: Preparation of 17-amino-6-hydroxy-6,15-bis(trifluoromethyl)-19-oxa-3,4,18-triazatricyclo[12.3.1.12,5]nonadeca-1(18),2,4,14,16-pentaene-10,13-dione (enantiomer 1) (Compound 36) and 17-amino-6-hydroxy-6,15-bis(trifluoromethyl)-19-oxa-3,4,18-triazatricyclo[12.3.1.12,5]nonadeca-1(18),2,4,14,16-pentaene-10,13-dione (Enantiomer 2) (Compound 37)

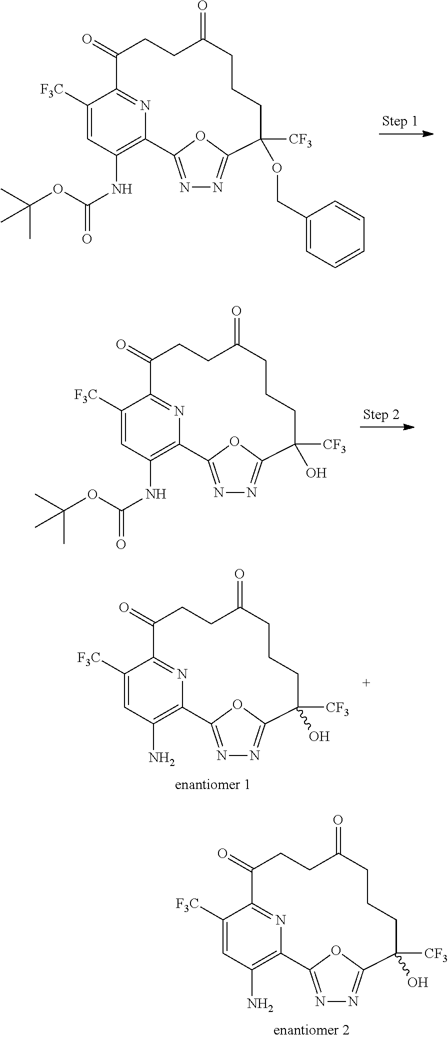 [SMILES: CC(C)(C)OC(=O)Nc1cc(C(F)(F)F)c2nc1C1=NN=C(O1)C(OCc1ccccc1)(C(F)(F)F)CCCC(=O)CCC2=O ; CC(C)(C)OC(=O)Nc1cc(C(F)(F)F)c2nc1C1=NN=C(O1)C(O)(C(F)(F)F)CCCC(=O)CCC2=O ; Nc1cc(C(F)(F)F)c2nc1C1=NN=C(O1)C(O)(C(F)(F)F)CCCC(=O)CCC2=O ; Nc1cc(C(F)(F)F)c2nc1C1=NN=C(O1)C(O)(C(F)(F)F)CCCC(=O)CCC2=O]

### Step 1: tert-Butyl N-[6-hydroxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate

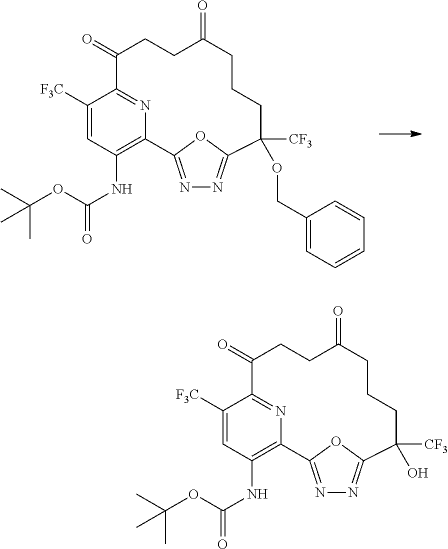

To a solution of tert-butyl N-[6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (70 mg, 0.1089 mmol) in acetic acid (1.89 mL) was added Pd/C (46.36 mg of 10% w/w, 0.04356 mmol). The mixture was stirred for 6 h under a hydrogen atmosphere using a Parr shaker at 150 psi. The reaction mixture was filtered through a silica plug, washing well with ethyl acetate and then concentrated to a white residue, which was purified by silica gel chromatography using a gradient from 0% to 30% ethyl acetate in hexanes over 20 minutes which gave as a white solid, tert-butyl N-[6-hydroxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (20 mg, 33%). ESI-MS m/z calc. 552.14435, found 553.34 (M+1)⁺; Retention time: 0.32 minutes (LC Method T).

### Step 2: 17-Amino-6-hydroxy-6,15-bis(trifluoromethyl)-19-oxa-3,4,18-triazatricyclo[12.3.1.12,5]nonadeca-1(18),2,4,14,16-pentaene-10,13-dione (enantiomer 1) (Compound 36) and 17-amino-6-hydroxy-6,15-bis(trifluoromethyl)-19-oxa-3,4,18-triazatricyclo[12.3.1.12,5]nonadeca-1(18),2,4,14,16-pentaene-10,13-dione (Enantiomer 2) (Compound 37)

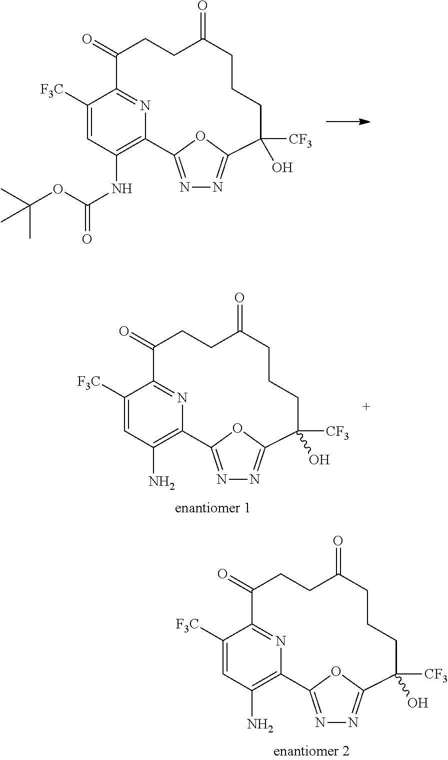 [SMILES: CC(C)(C)OC(=O)Nc1cc(C(F)(F)F)c2nc1C1=NN=C(O1)C(O)(C(F)(F)F)CCCC(=O)CCC2=O ; Nc1cc(C(F)(F)F)c2nc1C1=NN=C(O1)C(O)(C(F)(F)F)CCCC(=O)CCC2=O ; Nc1cc(C(F)(F)F)c2nc1C1=NN=C(O1)C(O)(C(F)(F)F)CCCC(=O)CCC2=O]

tert-Butyl N-[6-hydroxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (20 mg, 0.0362 mmol) was dissolved in dichloromethane (410.4 μL) and to the mixture was added TFA (167.2 μL, 2.17 mmol) and stirred at room temperature for 2 h. The reaction was concentrated and then purified by reverse phase chromatography using a gradient from 1% to 99% acetonitrile in water (+5 mM HCl). The resulting material was further purified by chiral SFC using IG column (250×21.2 mm, 5 m particle size) sold by Chiral Technologies with 5% to 15% methanol (20 mM NH₃) and carbon dioxide mobile phase over 14.5 minutes which gave as a white solid and the first enantiomer to elute, 17-amino-6-hydroxy-6,15-bis(trifluoromethyl)-19-oxa-3,4,18-triazatricyclo[12.3.1.12,5]nonadeca-1(18),2,4,14,16-pentaene-10,13-dione (enantiomer 1) (5 mg, 60%). ¹H NMR (400 MHz, CDCl₃) δ 7.57 (s, 1H), 6.27 (s, 2H), 3.94 (s, 1H), 3.58-3.45 (m, 2H), 3.03-2.85 (m, 2H), 2.73 (m, 1H), 2.46-2.32 (m, 3H), 2.12 (m, 1H), 1.78 (m, 1H) ppm. ESI-MS m/z calc. 452.09192, found 453.26 (M+1)⁺; Retention time: 1.37 minutes (LC Method A).

The later eluting enantiomer was isolated as a white solid, 17-amino-6-hydroxy-6,15-bis(trifluoromethyl)-19-oxa-3,4,18-triazatricyclo[12.3.1.12,5]nonadeca-1(18),2,4,14,16-pentaene-10,13-dione (enantiomer 2) (5 mg, 60%). ¹H NMR (400 MHz, CDCl₃) δ 7.57 (s, 1H), 6.27 (s, 2H), 4.41 (s, 1H), 3.59-3.43 (m, 2H), 3.03-2.86 (m, 2H), 2.76-2.69 (m, 1H), 2.48-2.31 (m, 3H), 2.18-2.07 (m, 1H), 1.78 (m, 1H) ppm. ESI-MS m/z calc. 452.09192, found 453.24 (M+1)⁺; Retention time: 1.37 minutes (LC Method A).

### Example 18: Preparation of (6R)-17-amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 38) and (6S)-17-amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 39)

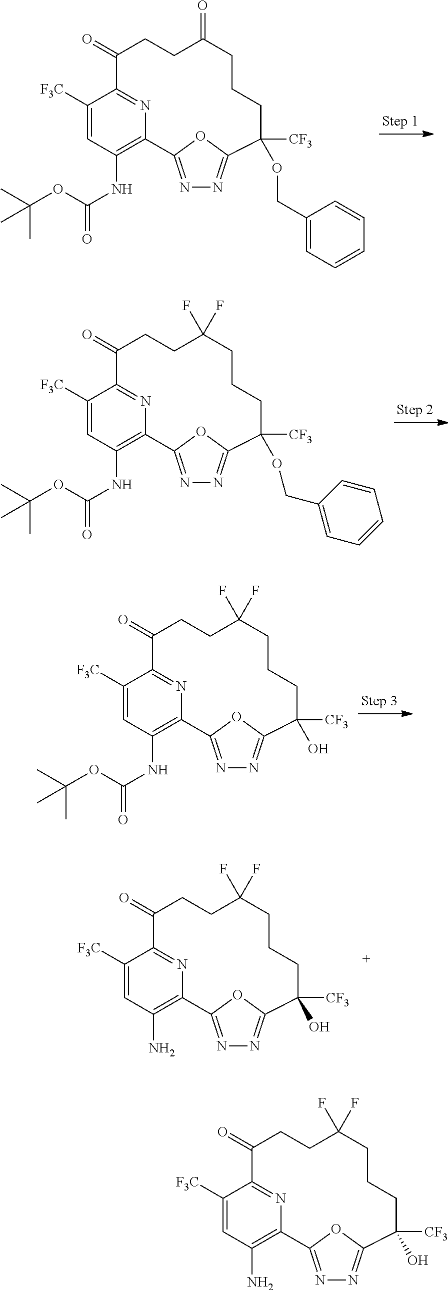 [SMILES: CC(C)(C)OC(=O)Nc1cc(C(F)(F)F)c2nc1C1=NN=C(O1)C(OCc1ccccc1)(C(F)(F)F)CCCC(=O)CCC2=O ; CC(C)(C)OC(=O)Nc1cc(C(F)(F)F)c2nc1C1=NN=C(O1)C(OCc1ccccc1)(C(F)(F)F)CCCC(F)(F)CCC2=O ; CC(C)(C)OC(=O)Nc1cc(C(F)(F)F)c2nc1C1=NN=C(O1)C(O)(C(F)(F)F)CCCC(F)(F)CCC2=O ; Nc1cc(C(F)(F)F)c2nc1C1=NN=C(O1)[C@@](O)(C(F)(F)F)CCCC(F)(F)CCC2=O ; Nc1cc(C(F)(F)F)c2nc1C1=NN=C(O1)[C@](O)(C(F)(F)F)CCCC(F)(F)CCC2=O]

### Step 1: tert-Butyl N-[6-benzyloxy-10,10-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate

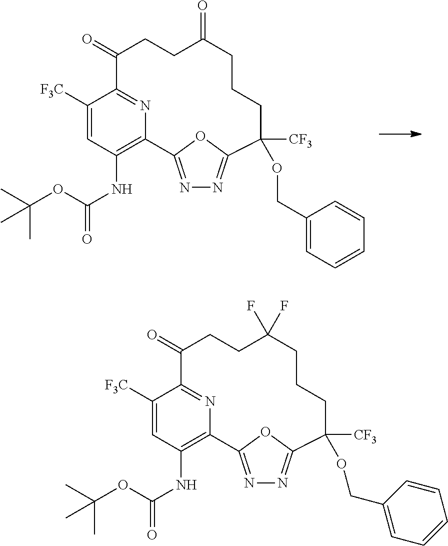 [SMILES: CC(C)(C)OC(=O)Nc1cc(C(F)(F)F)c2nc1C1=NN=C(O1)C(OCc1ccccc1)(C(F)(F)F)CCCC(=O)CCC2=O ; CC(C)(C)OC(=O)Nc1cc(C(F)(F)F)c2nc1C1=NN=C(O1)C(OCc1ccccc1)(C(F)(F)F)CCCC(F)(F)CCC2=O]

To glass vial under nitrogen a solution of tert-butyl N-[6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (114 mg, 0.1774 mmol) in 1,2-dichloroethane (354.8 μL) was added bis(2-methoxyethyl)aminosulfur trifluoride (392.5 μL of 50% w/v, 0.8870 mmol). Stirred reaction for 7 h at 50° C. Quenched with saturated aqueous NaHCO₃ and extracted with dichloromethane (2×15 mL). Combined the organic layers, washed with brine, dried over NaSO₄, filtered and concentrated. Purified by silica gel chromatography using a gradient from 0% to 30% ethyl acetate in hexanes which gave as a white solid, tert-butyl N-[6-benzyloxy-10,10-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (40 mg, 34%). ESI-MS m/z calc. 664.1932, found 665.42 (M+1)⁺; Retention time: 0.61 minutes (LC Method T).

### Step 2: tert-Butyl N-[10,10-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

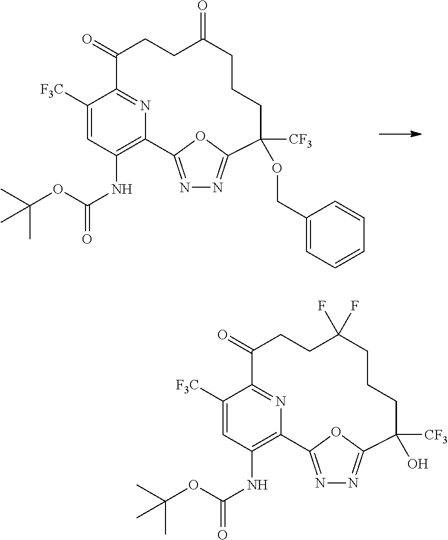

To a solution of tert-butyl N-[6-benzyloxy-10,10-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (55 mg, 0.08276 mmol) in acetic acid (1.485 mL) was added Pd/C (35.23 mg of 10% w/w, 0.0331 mmol). The mixture was stirred for 6 h under a hydrogen atmosphere using a Parr shaker at 150 psi. Reaction mixture was filtered through a silica plug, washing well with ethyl acetate and then concentrated, which was purified by silica gel chromatography using a gradient from 0% to 30% ethyl acetate in hexanes over 20 minutes which gave as a white solid, tert-butyl N-[10,10-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (18 mg, 76%). ESI-MS m/z calc. 574.14624, found 575.35 (M+1)⁺; Retention time: 0.38 minutes (LC Method T).

### Step 3: (6R)-17-Amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 38) and (6S)-17-amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 39)

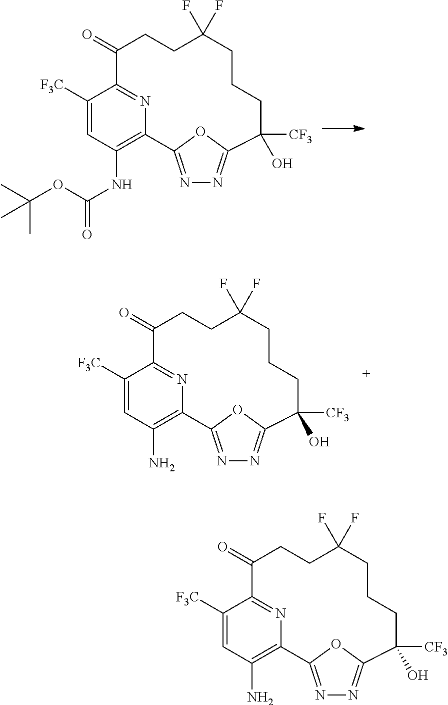 [SMILES: CC(C)(C)OC(=O)Nc1cc(C(F)(F)F)c2nc1C1=NN=C(O1)C(O)(C(F)(F)F)CCCC(F)(F)CCC2=O ; Nc1cc(C(F)(F)F)c2nc1C1=NN=C(O1)[C@@](O)(C(F)(F)F)CCCC(F)(F)CCC2=O ; Nc1cc(C(F)(F)F)c2nc1C1=NN=C(O1)[C@](O)(C(F)(F)F)CCCC(F)(F)CCC2=O]

tert-Butyl N-[10,10-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (27 mg, 0.047 mmol) was dissolved in dichloromethane (554.0 μL) and to the mixture was added TFA (217.1 μL, 2.818 mmol) and stirred at room temperature for 2 h. The reaction was concentrated and, then purified by reverse phase chromatography using 1% to 99% acetonitrile in water (+5 mM HCl). The resulting material was further purified by chiral SFC using IG column (250×21.2 mm, 5 m particle size) sold by Chiral Technologies with 5% to 15% methanol (20 mM NH₃) and carbon dioxide mobile phase over 14.5 minutes which gave as a white solid and the first enantiomer to elute, (6R)-17-amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (1.5 mg, 11%). ¹H NMR (400 MHz, CDCl₃) δ 7.58 (s, 1H), 6.23 (s, 2H), 3.65 (s, 1H), 3.27 (m, 1H), 3.09-2.98 (m, 1H), 2.92-2.78 (m, 1H), 2.66-2.32 (m, 3H), 2.32-1.97 (m, 3H), 1.85 (t, J=10.0 Hz, 1H) ppm. ESI-MS m/z calc. 474.0938, found 475.3 (M+1)⁺; Retention time: 1.66 minutes (LC Method A).

The later eluting enantiomer gave as a white solid, (6S)-17-amino-10,10-difluoro-6-hydroxy-6,15-bis(trifluoromethyl)-19-oxa-3,4,18-triazatricyclo[12.3.1.12,5]nonadeca-1(18),2,4,14,16-pentaen-13-one (1.5 mg, 11%). ¹H NMR (400 MHz, CDCl₃) δ 7.58 (s, 1H), 6.25 (s, 2H), 4.41 (s, 1H), 3.32-3.21 (m, 1H), 3.10-2.98 (m, 1H), 2.84 (s, 1H), 2.59-2.33 (m, 3H), 2.27-2.01 (m, 3H), 1.85 (dd, J=12.9, 6.7 Hz, 1H) ppm. ESI-MS m/z calc. 474.0938, found 475.3 (M+1)⁺; Retention time: 1.66 minutes (LC Method A).

### Example 19: Preparation of (6R)-17-amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 40) and (6S)-17-amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 41)

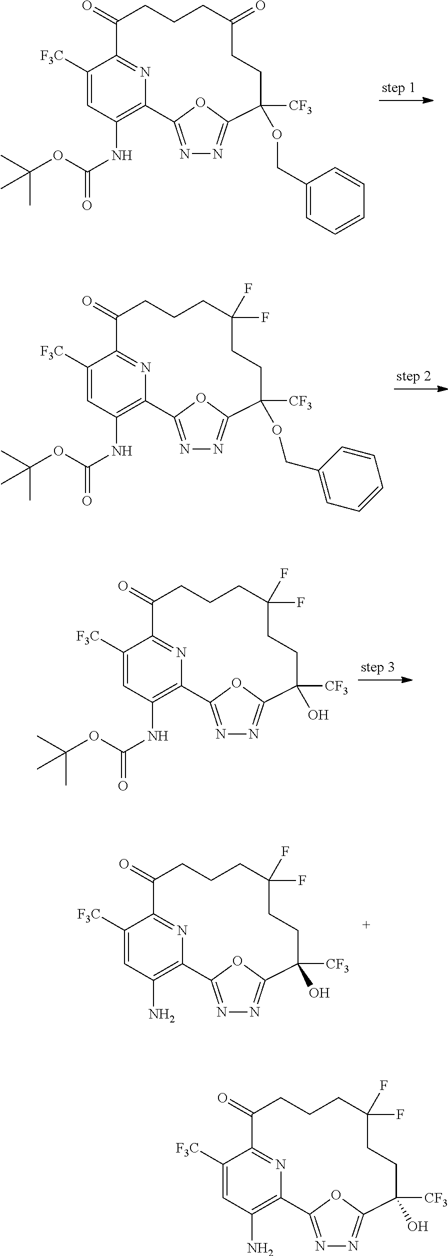 [SMILES: CC(C)(C)OC(=O)Nc1cc(C(F)(F)F)c2nc1C1=NN=C(O1)C(OCc1ccccc1)(C(F)(F)F)CCC(=O)CCCC2=O ; CC(C)(C)OC(=O)Nc1cc(C(F)(F)F)c2nc1C1=NN=C(O1)C(OCc1ccccc1)(C(F)(F)F)CCC(F)(F)CCCC2=O ; CC(C)(C)OC(=O)Nc1cc(C(F)(F)F)c2nc1C1=NN=C(O1)C(O)(C(F)(F)F)CCC(F)(F)CCCC2=O ; Nc1cc(C(F)(F)F)c2nc1C1=NN=C(O1)[C@@](O)(C(F)(F)F)CCC(F)(F)CCCC2=O ; Nc1cc(C(F)(F)F)c2nc1C1=NN=C(O1)[C@](O)(C(F)(F)F)CCC(F)(F)CCCC2=O]

### Step 1: tert-Butyl N-[6-benzyloxy-9,9-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate

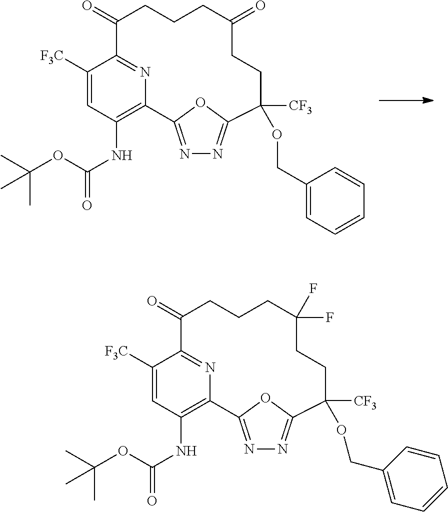 [SMILES: CC(C)(C)OC(=O)Nc1cc(C(F)(F)F)c2nc1C1=NN=C(O1)C(OCc1ccccc1)(C(F)(F)F)CCC(=O)CCCC2=O ; CC(C)(C)OC(=O)Nc1cc(C(F)(F)F)c2nc1C1=NN=C(O1)C(OCc1ccccc1)(C(F)(F)F)CCC(F)(F)CCCC2=O]

To a solution of tert-butyl N-[6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (295 mg, 0.4591 mmol) in 1,2-dichloroethane (5.5 mL) was slowly added diethylaminosulfur trifluoride (2.2 mL, 16.65 mmol) and the reaction mixture was stirred at room temperature for 30 minutes. Then the mixture was heated to 85° C. for 5 hours. The resultant mixture was quenched slowly at 0° C. with saturated aqueous NaHCO₃ (2 mL). The mixture was diluted with DCM, washed with water, dried over sodium sulfate, filtered and concentrated. The residue was purified by reverse phase chromatography using 30% to 99% acetonitrile and water (+5 mM HCl) over 15 minutes which gave as a white solid tert-butyl N-[6-benzyloxy-9,9-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (44.1 mg, 14%). ¹H NMR (400 MHz, CDCl₃) δ 9.81 (s, 1H), 9.39 (s, 1H), 7.30-7.20 (m, 5H), 4.94 (d, J=11.3 Hz, 1H), 4.84 (d, J=11.4 Hz, 1H), 3.19 (m, 1H), 2.73 (m, 2H), 2.52 (m, 2H), 2.34 (d, J=11.8 Hz, 2H), 2.17 (q, J=10.9, 8.2 Hz, 3H), 1.59 (s, 9H) ppm. ESI-MS m/z calc. 664.1932, found 665.2 (M+1)⁺; Retention time: 2.065 minutes (LC Method J).

### Step 2: tert-Butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

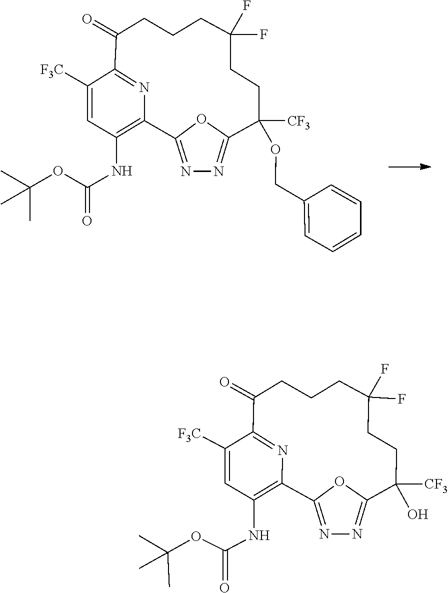 [SMILES: CC(C)(C)OC(=O)Nc1cc(C(F)(F)F)c2nc1-c1nnc(o1)C(OCc1ccccc1)(C(F)(F)F)CCC(F)(F)CCCC2=O ; CC(C)(C)OC(=O)Nc1cc(C(F)(F)F)c2nc1-c1nnc(o1)C(O)(C(F)(F)F)CCC(F)(F)CCCC2=O]

In a pressure reactor a solution of tert-butyl N-[6-benzyloxy-9,9-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (35 mg, 0.05267 mmol) in acetic acid (550 μL) and ethyl acetate (550 μL) was purged with nitrogen. Then, Pd/C (56.5 mg of 10% w/w, 0.05309 mmol) was added. The mixture was degassed with nitrogen, then filled with hydrogen gas and stirred at 180 psi for 4 hours. The reaction was filtered over Celite plug and washed with excess acetonitrile and ethyl acetate and concentrated. The residue was purified by silica gel chromatography (12 gram column) using a gradient from 100% hexanes to 60% ethyl acetate in hexanes (product elutes at 30% ethyl acetate) and placed under high vac pump for 2 hours which gave as a white solid, tert-butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (2 mg, 7%). ESI-MS m/z calc. 574.14624, found 575.2 (M+1)⁺; Retention time: 1.56 minutes (LC Method J) and tert-butyl N-[9,9-difluoro-6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (26 mg, 86%). ESI-MS m/z calc. 576.16187, found 577.2 (M+1)⁺; Retention time: 1.28 minutes (LC Method J) contaminated with a small amount of tert-butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate.

To a solution of the tert-butyl N-[9,9-difluoro-6,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (30 mg, 0.05204 mmol, contaminated with a small amount of tert-butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate) in dichloromethane (2.5 mL) at room temperature was added DMP (65 mg, 0.1533 mmol). After 30 minutes, added additional DMP (65 mg, 0.1533 mmol) and stirred additional 2.5 h. Quenched the reaction with saturated aqueous NaHCO₃ and extracted with dichloromethane (50 mL). Combined organic layers washed with brine, dried over sodium sulfate, filtered and concentrated. The residue was purified by silica gel chromatography (24 gram column) using a gradient from 100% hexanes to 70% ethyl acetate in hexanes (product elutes at 30% ethyl acetate) which gave as a white solid, tert-butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (28.5 mg, 95%). ESI-MS m/z calc. 574.14624, found 575.2 (M+1)⁺; Retention time: 1.56 minutes (LC Method J).

### Step 3: (6R)-17-Amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 40) and (6S)-17-amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 41)

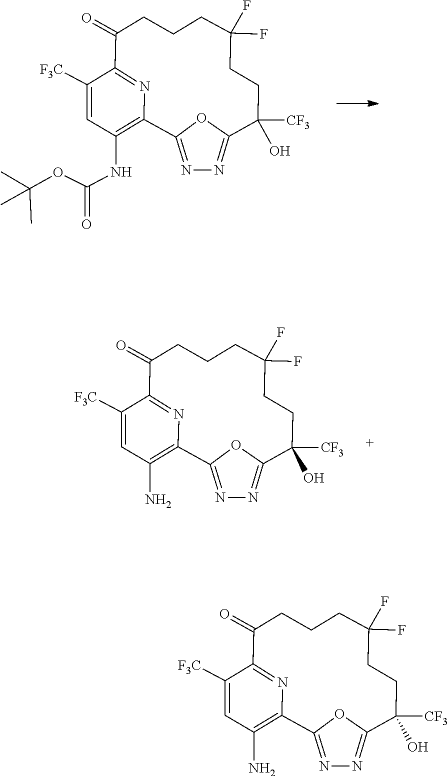 [SMILES: CC(C)(C)OC(=O)Nc1cc(C(F)(F)F)c2nc1-c1nnc(o1)C(O)(C(F)(F)F)CCC(F)(F)CCCC2=O ; Nc1cc(C(F)(F)F)c2nc1-c1nnc(o1)[C@@](O)(C(F)(F)F)CCC(F)(F)CCCC2=O ; Nc1cc(C(F)(F)F)c2nc1-c1nnc(o1)[C@](O)(C(F)(F)F)CCC(F)(F)CCCC2=O]

To a solution of tert-butyl N-[9,9-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (28.5 mg, 0.04962 mmol) in dichloromethane (750 μL) was added TFA (250 μL, 3.245 mmol) and stirred at room temperature for 60 mins. The mixture was evaporated to dryness, then diluted with ether and concentrated. The residue was purified by chiral SFC using IG column (250×21.2 mm, 5 m particle size) sold by Chiral Technologies with 5% to 15% methanol (20 mM NH₃) and carbon dioxide mobile phase over 14.5 minutes giving as the first enantiomer to elute, which was re-purified by reverse phase chromatography using 1% to 99% acetonitrile and water (+5 mM HCl) over 15 minutes which gave as a white solid, (6R)-17-amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (7.8 mg, 77%). ¹H NMR (400 MHz, CDCl₃) δ 7.55 (s, 1H), 6.20 (s, 2H), 3.79 (s, 1H), 3.14 (m, 1H), 2.85-2.73 (m, 1H), 2.59-2.50 (m, 1H), 2.46 (d, J=11.4 Hz, 1H), 2.41 (d, J=12.6 Hz, 2H), 2.26-2.16 (m, 2H), 2.15 (s, 1H), 2.12-2.07 (m, 1H) ppm. ESI-MS m/z calc. 474.0938, found 475.1 (M+1)⁺; Retention time: 1.66 minutes (LC Method A).

The later eluting peak was purified by reverse phase chromatography using 1% to 99% acetonitrile and water (+5 mM HCl) over 15 minutes which gave as a white solid, (6S)-17-amino-9,9-difluoro-6-hydroxy-6,15-bis(trifluoromethyl)-19-oxa-3,4,18-triazatricyclo[12.3.1.12,5]nonadeca-1(18),2,4,14,16-pentaen-13-one (7.8 mg, 77%). ¹H NMR (400 MHz, CDCl₃) δ 7.55 (s, 1H), 6.19 (s, 2H), 3.76 (s, 1H), 3.14 (m, 1H), 2.79 (m, 1H), 2.57-2.48 (m, 1H), 2.48-2.44 (m, 1H), 2.44-2.34 (m, 2H), 2.26-2.16 (m, 2H), 2.14 (d, J=7.1 Hz, 1H), 2.13-2.08 (m, 1H) ppm. ESI-MS m/z calc. 474.0938, found 475.2 (M+1)⁺; Retention time: 1.66 minutes (LC Method A).

### Example 20: Preparation of (6R)-17-amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-10-one (Compound 42)

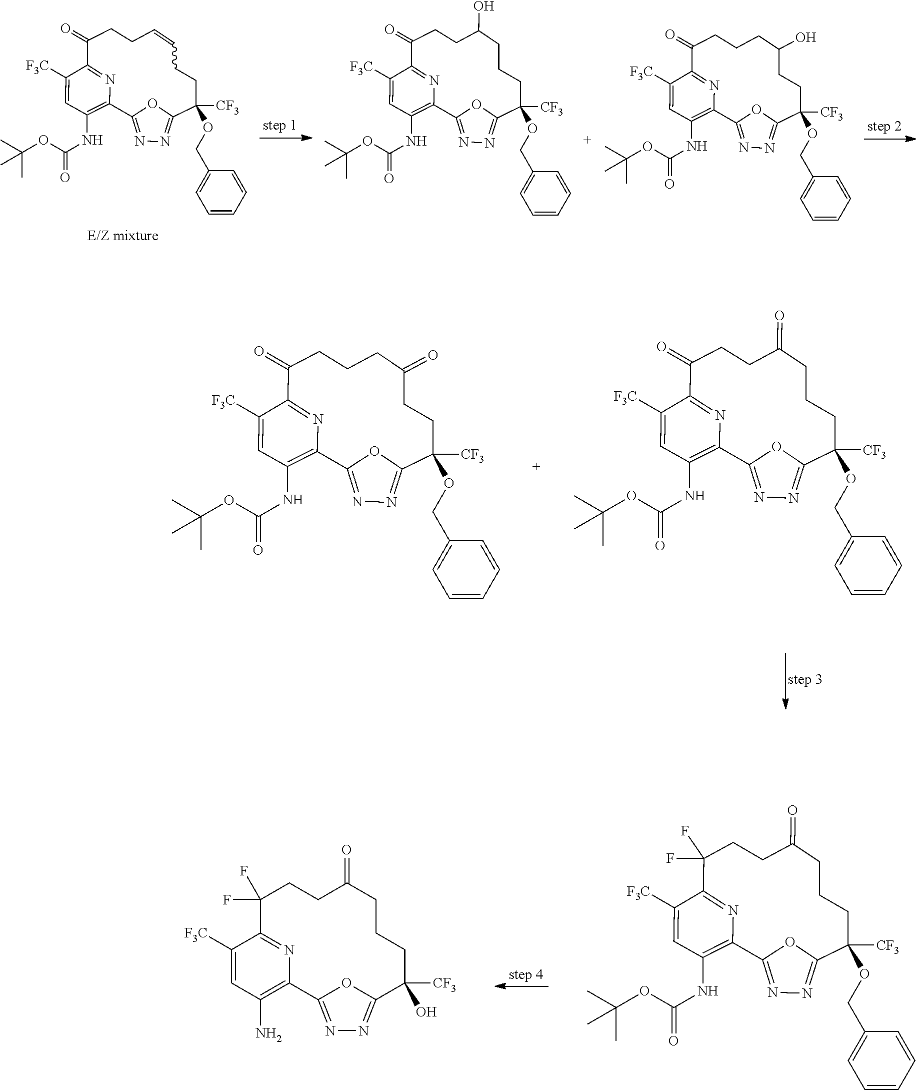

### Step 1: tert-Butyl N-1(6R)-6-benzyloxy-9,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate and tert-butyl N-[(6R)-6-benzyloxy-10,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate

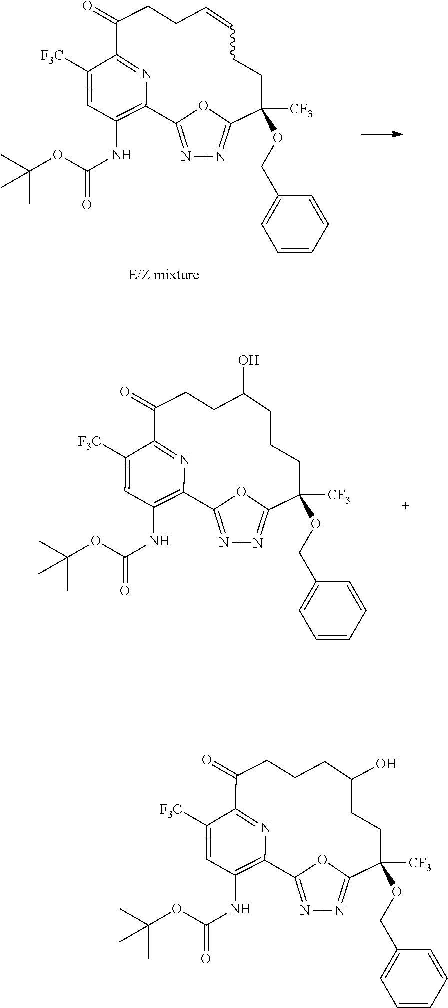 [SMILES: CC(C)(C)OC(=O)Nc1cc(C(F)(F)F)c2nc1-c1nnc(o1)[C@@](OCc1ccccc1)(C(F)(F)F)CCC=CCCC2=O ; CC(C)(C)OC(=O)Nc1cc(C(F)(F)F)c2nc1-c1nnc(o1)[C@@](OCc1ccccc1)(C(F)(F)F)CCCC(O)CCC2=O ; CC(C)(C)OC(=O)Nc1cc(C(F)(F)F)c2nc1-c1nnc(o1)[C@@](OCc1ccccc1)(C(F)(F)F)CCC(O)CCCC2=O]

To a solution of tert-butyl N-[(6R)-6-benzyloxy-13-oxo-6,15-bis(trifluoromethyl)-19-oxa-3,4,18-triazatricyclo[12.3.1.12,5]nonadeca-1(17),2,4,9,14(18),15-hexaen-17-yl]carbamate (E/Z mixture) (3.38 g, 5.395 mmol) in THF (25 mL) at 0° C. was added dropwise borane dimethyl sulfide solution (8.10 mL of 2 M in THF, 16.2 mmol) and let the resulting mixture stir for 15 min at 0° C. Next, the reaction was allowed to warm to room temperature and further stirred for 30 min until UPLC showed starting material was consumed. Cooled the reaction to 0° C. before quenching with aqueous NaOH (10.79 mL of 2 M, 21.58 mmol) followed by the addition of hydrogen peroxide (18.35 g, 161.8 mmol). Let the resulting mixture stir for 30 min at room temperature before extracting with ethyl acetate (2×50 mL). The organic layers were combined, washed with brine (50 mL), dried over sodium sulfate, filtered and concentrated. Purified the residue by silica gel chromatography using a gradient from 0% to 30% ethyl acetate in hexanes to afford as a white solid and mixture of diastereomers and regioisomers, tert-butyl N-[(6R)-6-benzyloxy-9,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate mixed with tert-butyl N-[(6R)-6-benzyloxy-10,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (1.175 g, 67%). ESI-MS m/z calc. 646.2226, found 647.27 (M+1)⁺; Retention time: 0.31 minutes (LC Method T).

### Step 2 tert-Butyl N-[(6R)-6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate and tert-butyl N-[(6R)-6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate

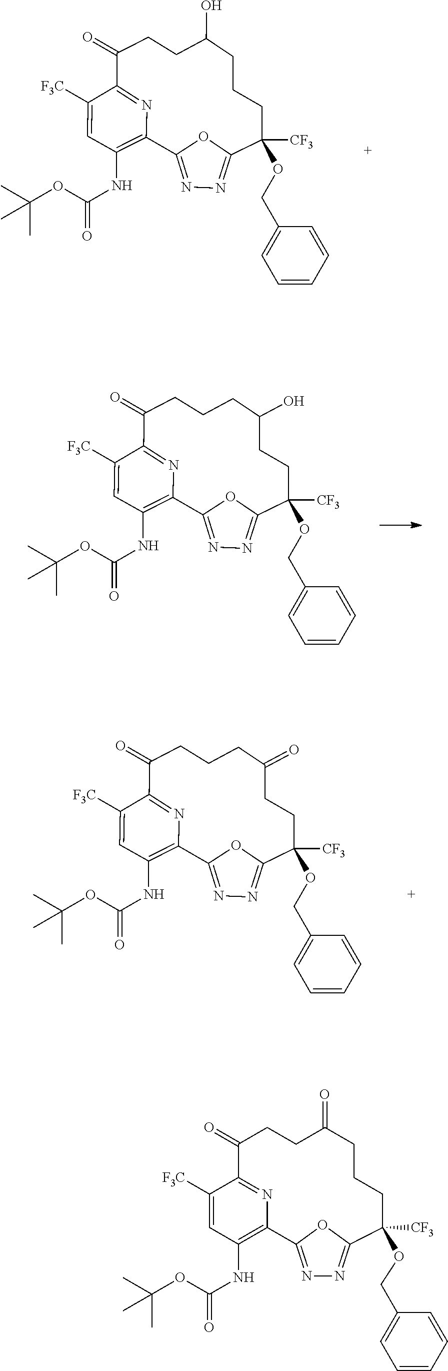 [SMILES: CC(C)(C)OC(=O)Nc1cc(C(F)(F)F)c2nc1-c1nnc(o1)[C@@](OCc1ccccc1)(C(F)(F)F)CCCC(O)CCC2=O ; CC(C)(C)OC(=O)Nc1cc(C(F)(F)F)c2nc1-c1nnc(o1)[C@@](OCc1ccccc1)(C(F)(F)F)CCC(O)CCCC2=O ; CC(C)(C)OC(=O)Nc1cc(C(F)(F)F)c2nc1-c1nnc(o1)[C@@](OCc1ccccc1)(C(F)(F)F)CCC(=O)CCCC2=O ; CC(C)(C)OC(=O)Nc1cc(C(F)(F)F)c2nc1C1=NN=C(O1)[C@@](OCc1ccccc1)(C(F)(F)F)CCCC(=O)CCC2=O]

To a solution of a mixture of regioisomers/diasteromers, tert-butyl N-[(6R)-6-benzyloxy-10,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate and tert-butyl N-[(6R)-6-benzyloxy-9,13-dihydroxy-6,15-bis(trifluoromethyl)-19-oxa-3,4,18-triazatricyclo[12.3.1.12,5]nonadeca-1(17),2,4,14(18),15-pentaen-17-yl]carbamate (2.35 g, 3.635 mmol) in dichloromethane (44 mL) was added Dess-Martin periodinane (4.623 g, 10.9 mmol). The reaction was stirred for 30 min then quenched with saturated aqueous NaHCO₃ and extracted with dichloromethane (2×50 mL). The organic layers were combined and washed with brine, dried over NaSO₄, filtered and concentrated. The residue was purified by silica gel chromatography using a gradient from 0% to 10% ethyl acetate in hexanes to afford as the first regioisomer to elute, tert-butyl N-[(6R)-6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (1.1 g, 47%). ESI-MS m/z calc. 642.1913, found 643.29 (M+1)⁺; Retention time: 0.59 minutes (LC Method T).

The second regioisomer to elute was isolated as tert-butyl N-[(6R)-6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (583 mg, 25%). ESI-MS m/z calc. 642.1913, found 643.29 (M+1)⁺; Retention time: 0.57 minutes (LC Method T).

### Step 3: tert-Butyl N-[(6R)-6-benzyloxy-13,13-difluoro-10-oxo-6,15-bis(trifluoromethyl)-19-oxa-3,4,18-triazatricyclo[12.3.1.12,5]nonadeca-1(17),2,4,14(18),15-pentaen-17-yl]carbamate

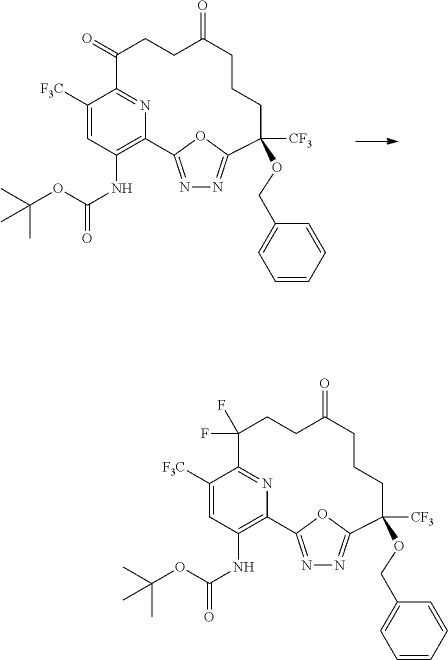 [SMILES: CC(C)(C)OC(=O)Nc1cc(C(F)(F)F)c2nc1C1=NN=C(O1)[C@@](OCc1ccccc1)(C(F)(F)F)CCCC(=O)CCC2=O ; CC(C)(C)OC(=O)Nc1cc(C(F)(F)F)c2nc1C1=NN=C(O1)[C@@](OCc1ccccc1)(C(F)(F)F)CCCC(=O)CCC2(F)F]

To a glass sealed vial under nitrogen containing a solution of tert-butyl N-[(6R)-6-benzyloxy-10,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (350 mg, 0.5447 mmol) in 1,2-dichloroethane (1.09 mL) was added bis(2-methoxyethyl)aminosulfur trifluoride (2.41 mL of 50% w/v in THF, 5.447 mmol). Stirred the reaction for 6 h at 50° C. then quenched with saturated aqueous NaHCO₃ and extracted with dichloromethane (2×25 mL). Combined the organic layers, washed with brine, dried over NaSO₄, filtered and concentrated then purified via silica gel chromatography using a gradient from 0% to 30% ethyl acetate in hexanes to afford as a white solid, tert-butyl N-[(6R)-6-benzyloxy-13,13-difluoro-10-oxo-6,15-bis(trifluoromethyl)-19-oxa-3,4,18-triazatricyclo[12.3.1.12,5]nonadeca-1(17),2,4,14(18),15-pentaen-17-yl]carbamate (165 mg) which was contaminated with a regioisomeric product and taken directly to the next step. ESI-MS m/z calc. 664.1932, found 665.22 (M+1)⁺; Retention time: 0.65 minutes (LC Method T).

### Step 4: (6R)-17-Amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-10-one (Compound 42)

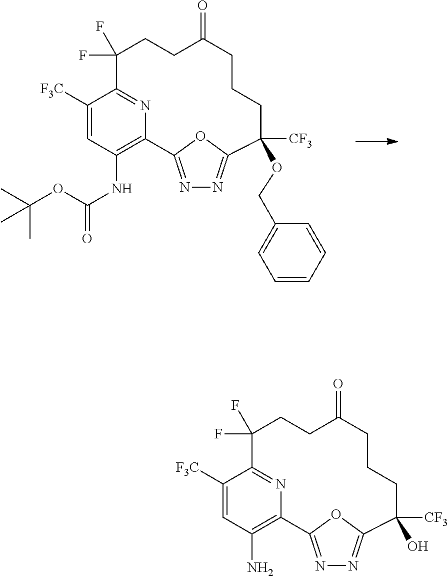 [SMILES: CC(C)(C)OC(=O)Nc1cc(C(F)(F)F)c2nc1C1=NN=C(O1)[C@@](OCc1ccccc1)(C(F)(F)F)CCCC(=O)CCC2(F)F ; Nc1cc(C(F)(F)F)c2nc1C1=NN=C(O1)[C@@](O)(C(F)(F)F)CCCC(=O)CCC2(F)F]

Part 1: To a solution of tert-butyl N-[(6R)-6-benzyloxy-13,13-difluoro-10-oxo-6,15-bis(trifluoromethyl)-19-oxa-3,4,18-triazatricyclo[12.3.1.12,5]nonadeca-1(17),2,4,14(18),15-pentaen-17-yl]carbamate (165 mg) which was contaminated with a regioisomeric product (as described in the previous step) in acetic acid (4.455 mL) was added 10% Pd/C (105.7 mg, 0.09932 mmol). The mixture was stirred for 6 h under a hydrogen atmosphere using a parr shaker at 150 psi. The reaction mixture was filtered through a silica plug, washing well with ethyl acetate and then the filtrated was concentrated to a white residue which was purified by silica chromatography using a gradient from 0% to 30% ethyl acetate in hexanes over 20 minutes to obtain a crude mixture of regioisomeric intended products and some over-reduced side-product. Carried this mixture forward to the next part.

Part 2: To the mixture obtained in part 1 (80 mg) in dichloromethane (1.107 mL) was added Dess-Martin periodinane (76 mg, 0.1792 mmol) and the resulting mixture was stirred for 15 min. Quenched the reaction with saturated aqueous NaHCO₃ then extracted with dichloromethane (2×25 mL). Combined the organic layers and washed with brine, dried over NaSO₄, filtered and concentrated then purified by silica gel chromatography using a gradient from 0% to 30% ethyl acetate in hexanes to afford a regioisomeric mixture of products which was taken directly to the next part.

Part 3: To the mixture obtained in part 2 was added TFA (563 μL, 7.308 mmol) and the resulting solution was stirred at room temperature for 2 h. The reaction was concentrated then purified by reverse phase HPLC using a gradient from 1% to 99% acetonitrile in water. Fractions of the minor regioisomer isolated from this purification were combined and washed with saturated aqueous NaHCO₃ then brine, dried over NaSO₄, filtered and then concentrated to afford as a white solid, (6R)-17-amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-10-one (6 mg, 2%, 2 steps). ¹H NMR (400 MHz, Chloroform-d) δ 7.51 (s, 1H), 5.96 (s, 2H), 3.94 (s, 1H), 3.39-3.22 (m, 1H), 2.95-2.82 (m, 1H), 2.79-2.68 (m, 1H), 2.62 (m, 1H), 2.54 (d, J=10.7 Hz, 1H), 2.32-2.19 (m, 3H), 2.06-1.98 (m, 1H), 1.71-1.60 (m, 1H) ppm. ESI-MS m/z calc. 474.0938, found 475.04 (M+1)⁺; Retention time: 0.65 minutes (LC Method S).

### Example 21: Preparation of (6R)-17-amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-9-one (Compound 43)

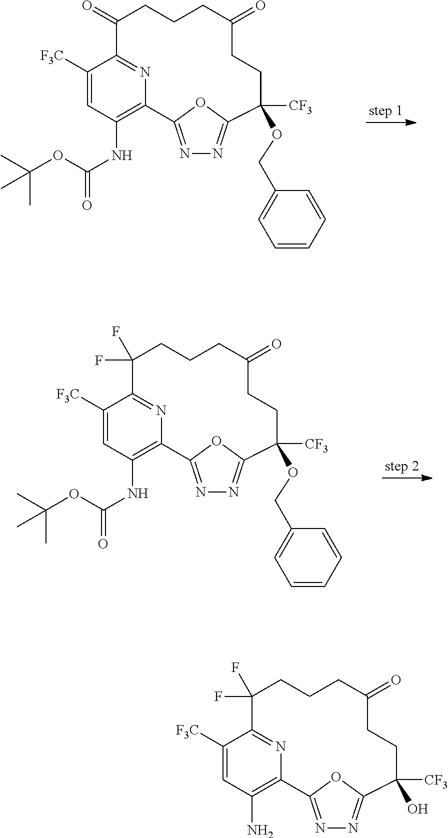 [SMILES: CC(C)(C)OC(=O)Nc1cc(C(F)(F)F)c2nc1C1=NN=C(O1)[C@@](OCc1ccccc1)(C(F)(F)F)CCC(=O)CCCC2=O ; CC(C)(C)OC(=O)Nc1cc(C(F)(F)F)c2nc1C1=NN=C(O1)[C@@](OCc1ccccc1)(C(F)(F)F)CCC(=O)CCCC2(F)F ; Nc1cc(C(F)(F)F)c2nc1C1=NN=C(O1)[C@@](O)(C(F)(F)F)CCC(=O)CCCC2(F)F]

### Step 1: tert-Butyl N-[(6R)-6-benzyloxy-13,13-difluoro-9-oxo-6,15-bis(trifluoromethyl)-19-oxa-3,4,18-triazatricyclo[12.3.1.12,5]nonadeca-1(17),2,4,14(18),15-pentaen-17-yl]carbamate

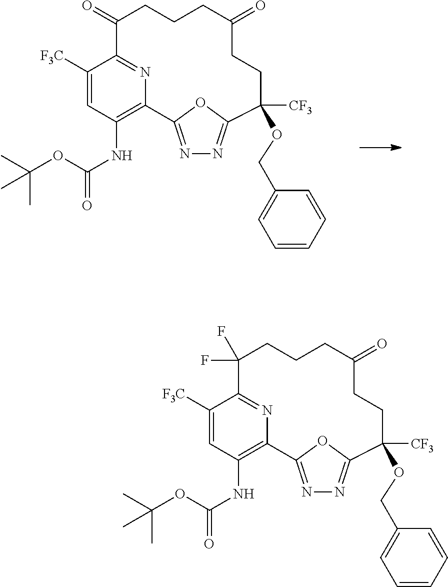 [SMILES: CC(C)(C)OC(=O)Nc1cc(C(F)(F)F)c2nc1C1=NN=C(O1)[C@@](OCc1ccccc1)(C(F)(F)F)CCC(=O)CCCC2=O ; CC(C)(C)OC(=O)Nc1cc(C(F)(F)F)c2nc1C1=NN=C(O1)[C@@](OCc1ccccc1)(C(F)(F)F)CCC(=O)CCCC2(F)F]

In a 20 mL sealed vial, tert-butyl N-[(6R)-6-benzyloxy-9,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (1.02 g, 1.587 mmol) was dissolved in 1,2-dichloroethane (9 mL) at room temperature. To the stirring solution was slowly added diethylaminosulfur trifluoride (6.75 mL, 51.09 mmol) allowing to stir at room temperature for 15 additional minutes. Then, the mixture was capped and heated to 85° C. for 5 h behind a blast shield. The resultant mixture was quenched slowly at 0° C. by addition of saturated aqueous NaHCO₃ (20 mL). The mixture was diluted with dichloromethane, washed with water, and dried over sodium sulfate then filtered and concentrated. The crude filtrate was purified by silica gel chromatography using a gradient from 100% hexanes to 50% ethyl acetate in hexanes to afford the product containing impurities as a tan solid. The mixture was further purified by reverse-phase preparative chromatography utilizing a C₁₈ column and a gradient from 30% to 99% acetonitrile in water (+5 mM HCl) to afford as a white solid, tert-butyl N-[(6R)-6-benzyloxy-13,13-difluoro-9-oxo-6,15-bis(trifluoromethyl)-19-oxa-3,4,18-triazatricyclo[12.3.1.12,5]nonadeca-1(17),2,4,14(18),15-pentaen-17-yl]carbamate (150 mg) which was contaminated with a regioisomeric product. ESI-MS m/z calc. 664.1932, found 665.2 (M+1)⁺; Retention time: 2.08 minutes (LC Method J). This material was taken directly to the ensuing step.

### Step 2: (6R)-17-Amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-9-one (Compound 43)

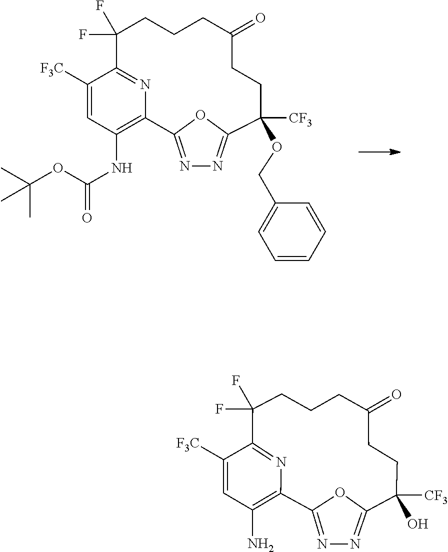 [SMILES: CC(C)(C)OC(=O)Nc1cc(C(F)(F)F)c2nc1C1=NN=C(O1)[C@@](OCc1ccccc1)(C(F)(F)F)CCC(=O)CCCC2(F)F ; Nc1cc(C(F)(F)F)c2nc1-c1nnc(o1)[C@@](O)(C(F)(F)F)CCC(=O)CCCC2(F)F]

Part 1: In a pressure reactor, a solution of tert-butyl N-[(6R)-6-benzyloxy-13,13-difluoro-9-oxo-6,15-bis(trifluoromethyl)-19-oxa-3,4,18-triazatricyclo[12.3.1.12,5]nonadeca-1(17),2,4,14(18),15-pentaen-17-yl]carbamate (150 mg) (which was contaminated with a regioisomeric product as described in step 1) in acetic acid (2.5 mL) and ethyl acetate (2.5 mL) was purged with nitrogen. Then 10% Pd/C (235 mg, 0.2208 mmol) was added. The mixture was degassed with nitrogen, then filled with hydrogen gas and stirred at 180 psi for 4 h. The mixture was degassed with nitrogen then filled with hydrogen gas and stirred at 180 psi for 2 additional hours. The reaction was filtered, washing the celite plug with excess acetonitrile and ethyl acetate and the filtrate was concentrated. The crude residue obtained was then purified by silica gel chromatography using a gradient from 100% hexanes to 60% ethyl acetate in hexanes to afford a crude mixture of regioisomeric intended products and some over-reduced side-product (108 mg). Carried this mixture forward to the next part.

Part 2: To a solution of the mixture of products obtained in part 1 (108 mg) in dichloromethane (2.5 mL) was added Dess-Martin periodinane (150 mg, 0.3537 mmol). After 15 minutes, quenched the reaction with saturated aqueous NaHCO₃ then extracted with dichloromethane (50 mL). Washed the organic dichloromethane layer with brine, dried over sodium sulfate, filtered and concentrated. The crude filtrate was then purified by silica gel chromatography using a gradient from 100% hexanes to 65% ethyl acetate in hexanes to afford a regioisomeric mixture of products as a white solid which was taken directly to the next part.

Part 3: To the mixture obtained in part 2 was added TFA (500 μL, 6.49 mmol) and the resulting mixture was stirred at room temperature for 60 minutes. The mixture was evaporated to dryness then diluted with ether and concentrated. The crude material obtained was then purified by silica gel chromatography using a gradient from 100% dichloromethane to 15% methanol in dichloromethane and concentrated. The isolated mixture was further purified by reverse-phase preparative chromatography utilizing a C18 column and a gradient from 1% to 99% acetonitrile in water (+5 mM HCl) to afford as a white solid, (6R)-17-amino-13,13-difluoro-6-hydroxy-6,15-bis(trifluoromethyl)-19-oxa-3,4,18-triazatricyclo[12.3.1.12,5]nonadeca-1(18),2,4,14,16-pentaen-9-one (4.8 mg, 7%). ¹H NMR (500 MHz, DMSO-d₆) δ 7.89 (s, 1H), 7.82 (s, 1H), 7.36 (s, 2H), 3.22-3.15 (m, 1H), 2.86-2.80 (m, 1H), 2.80-2.76 (m, 1H), 2.40 (m, 4H), 2.28 (m, 1H), 1.78-1.70 (m, 1H), 1.61 (m, 1H) ppm. ESI-MS m/z calc. 474.0938, found 475.2 (M+1)⁺; Retention time: 1.64 minutes (LC Method A).

### Example 22: Preparation of (6R)-17-amino-8,8-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 44)

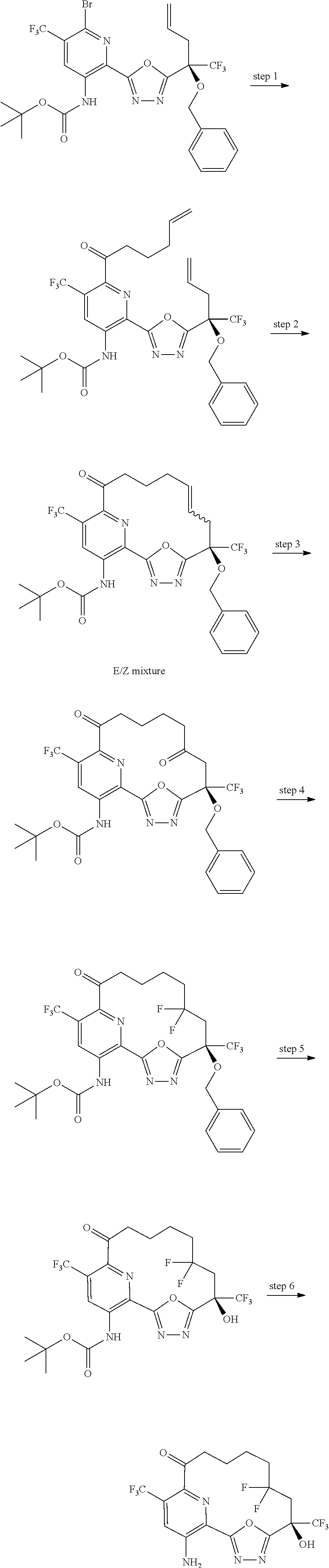

### Step 1: tert-Butyl N-[2-[5-[(1R)-1-benzyloxy-1-(trifluoromethyl)but-3-enyl]-1,3,4-oxadiazol-2-yl]-6-hex-5-enoyl-5-(trifluoromethyl)-3-pyridyl]carbamate

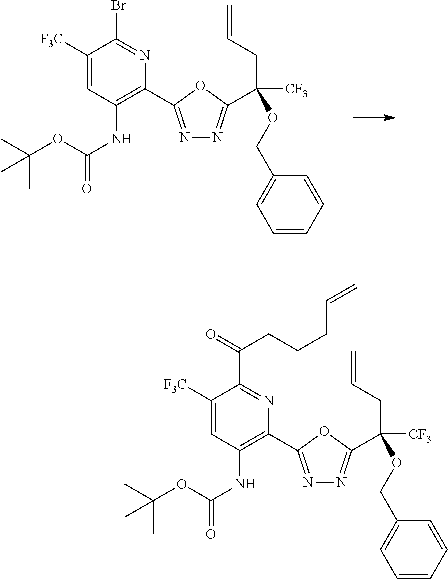 [SMILES: C=CC[C@@](OCc1ccccc1)(c1nnc(-c2nc(Br)c(C(F)(F)F)cc2NC(=O)OC(C)(C)C)o1)C(F)(F)F ; C=CCCCC(=O)c1nc(-c2nnc([C@@](CC=C)(OCc3ccccc3)C(F)(F)F)o2)c(NC(=O)OC(C)(C)C)cc1C(F)(F)F]

To tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (1.2 g, 1.883 mmol) in ether (19 mL) at −78° C. was added n-BuLi (1.66 mL of 2.5 M, 4.15 mmol) as a solution in hexanes. After stirring at −78° C. for 5 min, more n-BuLi (90 μL of 2.5 M, 0.225 mmol) as a solution in hexanes was added. After stirring a further 15 min at −78° C., a solution of N-methoxy-N-methyl-hex-5-enamide (416 mg, 2.646 mmol) in ether (6 mL) was added dropwise. The mixture was stirred at −78° C. for 15 min and then at 0° C. for 15 min. Then, the mixture was diluted with 1 M NH₄Cl in water (10 mL) and ether then partitioned. The organic layer was separated and washed with 1 M NaHCO₃, dried (MgSO₄), filtered and evaporated. The residue was purified by silica gel chromatography using a gradient from 0% to 30% of a solution (10% ethyl acetate in hexanes) to hexanes over 18 min which provided tert-butyl N-[2-[5-[(1R)-1-benzyloxy-1-(trifluoromethyl)but-3-enyl]-1,3,4-oxadiazol-2-yl]-6-hex-5-enoyl-5-(trifluoromethyl)-3-pyridyl]carbamate (575 mg, 47%). ¹H NMR (400 MHz, Chloroform-d) δ 10.32 (s, 1H), 9.41 (s, 1H), 7.44-7.28 (m, 5H), 5.94 (m, J=17.1, 7.3 Hz, 1H), 5.79 (m, J=16.9, 10.1, 6.7 Hz, 1H), 5.27 (dd, J=17.2, 1.6 Hz, 1H), 5.21 (dd, J=10.2, 1.4 Hz, 1H), 5.02 (dd, J=17.1, 1.8 Hz, 1H), 4.97 (dd, J=10.4, 1.8 Hz, 1H), 4.85 (d, J=10.8 Hz, 1H), 4.71 (d, J=10.9 Hz, 1H), 3.35-3.16 (m, 2H), 3.13 (t, J=7.3 Hz, 2H), 2.14 (q, J=7.2 Hz, 2H), 1.85 (p, J=7.3 Hz, 2H), 1.58 (s, 9H) ppm; ¹⁹F NMR (376 MHz, Chloroform-d) δ−59.95 (d, J=1.7 Hz), −73.15 ppm. ESI-MS m/z calc. 654.22766, found 655.2 (M+1)⁺; Retention time: 0.63 minutes (LC Method T).

### Step 2: tert-Butyl N-[(6R)-6-benzyloxy-13-oxo-6,15-bis(trifluoromethyl)-19-oxa-3,4,18-triazatricyclo[12.3.1.12,5]nonadeca-1(17),2,4,8,14(18),15-hexaen-17-yl]carbamate (E/Z Mixture)

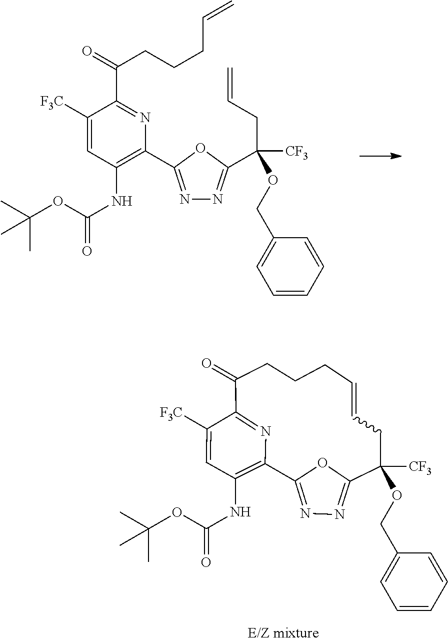 [SMILES: C=CCCCC(=O)c1nc(-c2nnc([C@@](CC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1-c1nnc(o1)[C@@](OCc1ccccc1)(C(F)(F)F)CC=CCCCC2=O]

A solution of tert-butyl N-[2-[5-[(1R)-1-benzyloxy-1-(trifluoromethyl)but-3-enyl]-1,3,4-oxadiazol-2-yl]-6-hex-5-enoyl-5-(trifluoromethyl)-3-pyridyl]carbamate (215 mg, 0.2792 mmol) in toluene (14 mL) was added dropwise over 5 min to a 120° C. preheated solution of [1,3-bis-(2-tolyl)-2-imidazolidinylidene]dichloro(2-isopropoxybenzylidene)ruthenium(II) (24 mg, 0.04207 mmol) in toluene (14 mL) which had a slow stream of nitrogen bubbled into it. With continued nitrogen bubbling, the mixture was stirred at 120° C. for 1 h, then additional catalyst, [1,3-bis-(2-tolyl)-2-imidazolidinylidene]dichloro(2-isopropoxybenzylidene)ruthenium(II) (10 mg, 0.01753 mmol) as a solution in toluene (1 mL) was added and the mixture stirred at 120° C. for 30 min further. Then, the solvent was evaporated and the residue purified silica gel chromatography using a gradient from 0% to 50% of a solution (10% ethyl acetate in hexanes) to hexanes over 25 min to provide tert-butyl N-[(6R,8Z)-6-benzyloxy-13-oxo-6,15-bis(trifluoromethyl)-19-oxa-3,4,18-triazatricyclo[12.3.1.12,5]nonadeca-1(17),2,4,8,14(18),15-hexaen-17-yl]carbamate (24 mg, 14%). ¹H NMR (400 MHz, Chloroform-d) δ 9.97 (s, 1H), 9.37 (s, 1H), 7.38-7.27 (m, 5H), 5.66-5.52 (m, 2H), 5.00 (t, J=11.0 Hz, 2H), 3.24 (s, 1H), 3.14 (q, J=8.9, 7.2 Hz, 1H), 3.03 (m, J=13.9, 7.2 Hz, 1H), 2.87 (m, J=12.8, 6.6 Hz, 1H), 2.73 (s, 1H), 2.49 (s, 1H), 2.21-2.06 (m, 1H), 1.98 (m, J=13.2, 6.8 Hz, 1H), 1.57 (s, 9H) ppm; ¹⁹F NMR (376 MHz, Chloroform-d) δ−60.52, −74.54 ppm. ESI-MS m/z calc. 626.1964, found 627.2 (M+1)⁺; Retention time: 0.56 minutes (LC Method T).

Continued elution provided tert-butyl N-[(6R,8E)-6-benzyloxy-13-oxo-6,15-bis(trifluoromethyl)-19-oxa-3,4,18-triazatricyclo[12.3.1.12,5]nonadeca-1(17),2,4,8,14(18),15-hexaen-17-yl]carbamate (73 mg, 42%). ¹H NMR (400 MHz, Chloroform-d) δ 10.01 (s, 1H), 9.38 (s, 1H), 7.33-7.19 (m, 4H), 7.18-7.05 (m, 1H), 6.14-5.98 (m, 1H), 5.83 (m, J=14.8, 8.9, 5.2 Hz, 1H), 4.91 (d, J=11.7 Hz, 1H), 4.61 (d, J=11.7 Hz, 1H), 3.32-3.15 (m, 2H), 2.89 (m, J=11.0, 5.7 Hz, 1H), 2.81 (dd, J=14.6, 8.9 Hz, 1H), 2.38-2.15 (m, 2H), 2.03-1.83 (m, 2H), 1.58 (s, 9H) ppm; ¹⁹F NMR (376 MHz, Chloroform-d) δ−60.84, −74.54 ppm. ESI-MS m/z calc. 626.1964, found 627.2 (M+1)⁺; Retention time: 0.56 minutes (LC Method T).

The cis and trans products obtained were combined to give tert-butyl N-[(6R)-6-benzyloxy-13-oxo-6,15-bis(trifluoromethyl)-19-oxa-3,4,18-triazatricyclo[12.3.1.12,5]nonadeca-1(17),2,4,8,14(18),15-hexaen-17-yl]carbamate (E/Z mixture) which was used directly in the next step.

### Step 3: tert-Butyl N-[(6R)-6-benzyloxy-8,13-dioxo-6,15-bis(trifluoromethyl)-19-oxa-3,4,18-triazatricyclo[12.3.1.12,5]nonadeca-1(17),2,4,14(18),15-pentaen-17-yl]carbamate

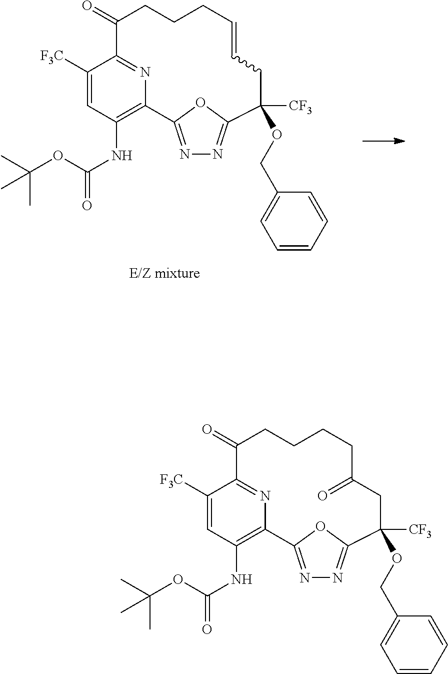 [SMILES: CC(C)(C)OC(=O)Nc1cc(C(F)(F)F)c2nc1C1=NN=C(O1)[C@@](OCc1ccccc1)(C(F)(F)F)CC=CCCCC2=O ; CC(C)(C)OC(=O)Nc1cc(C(F)(F)F)c2nc1C1=NN=C(O1)[C@@](OCc1ccccc1)(C(F)(F)F)CC(=O)CCCCC2=O]

Part 1: To a solution of tert-butyl N-[(6R)-6-benzyloxy-13-oxo-6,15-bis(trifluoromethyl)-19-oxa-3,4,18-triazatricyclo[12.3.1.12,5]nonadeca-1(17),2,4,8,14(18),15-hexaen-17-yl]carbamate (E/Z mixture) (400 mg, 0.6384 mmol) in THF (12 mL) at 0° C. was added dropwise borane dimethyl sulfide solution (500 μL of 2 M in THF, 1 mmol) and the mixture was stirred for 15 min at 0° C. Allowed the reaction to warm to room temperature and stirred for 1 h. Added additional borane dimethyl sulfide solution (500 μL of 2 M in THF, 1 mmol) and stirred at room temperature for 30 more minutes. Cooled the reaction to 0° C. before quenching with NaOH (2.95 mL of 1 M, 2.95 mmol) followed by the addition of hydrogen peroxide (2.55 mL of 30% w/v, 22.49 mmol). Let the mixture stir for 30 min at room temperature then extracted with ethyl acetate (2×80 mL). The organic layers were combined, washed with brine (80 mL), dried over sodium sulfate, filtered and concentrated. The crude material was then purified by silica gel chromatography using a gradient from 100% hexanes to 80% ethyl acetate in hexanes to afford the intermediate product as a white solid comprised of a mixture of isomers.

Part 2: To a solution of the mixture obtained in part 1 in dichloromethane (5.5 mL) was added Dess-Martin periodinane (290 mg, 0.6837 mmol). After 15 min, added more Dess-Martin periodinane (290 mg, 0.6837 mmol) and stirred additional 15 minutes. Quenched the reaction with saturated aqueous NaHCO₃ and extracted with dichloromethane (50 mL). The organic layer was washed with brine, dried over sodium sulfate, filtered and concentrated. The crude material was then purified by silica gel chromatography using a gradient from 100% hexanes to 65% ethyl acetate in hexanes to afford a mixture of two regioisomeric products as a white solid. Further purification was performed using a normal phase SFC-MS method (IC column, 250×21.2 mm, 5 μm particle size sold by Chiral Technologies and a dual gradient run from 5% to 15% mobile phase B over 14.5 minutes (mobile phase A=CO₂, mobile phase B=MeOH (+20 mM NH₃), column temperature=40° C.). The second regioisomer to elute (major product) was isolated as a white solid, tert-butyl N-[(6R)-6-benzyloxy-8,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (210 mg, 51%, 2 steps). ¹H NMR (400 MHz, Chloroform-d) δ 9.92 (s, 1H), 9.39 (s, 1H), 7.42-7.35 (m, 2H), 7.35-7.25 (m, 3H), 5.04 (d, J=10.6 Hz, 1H), 4.85 (d, J=10.6 Hz, 1H), 3.58 (d, J=17.0 Hz, 1H), 3.36-3.22 (m, 2H), 3.05 (m, 1H), 2.74-2.63 (m, 1H), 2.54 (m, 1H), 2.20 (m, 1H), 2.03-1.84 (m, 2H), 1.65 (m, 1H), 1.57 (s, 9H) ppm. ESI-MS m/z calc. 642.1913, found 643.2 (M+1)⁺; Retention time: 1.99 minutes (LC Method J).

### Step 4: tert-Butyl N-[(6R)-6-benzyloxy-8,8-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate

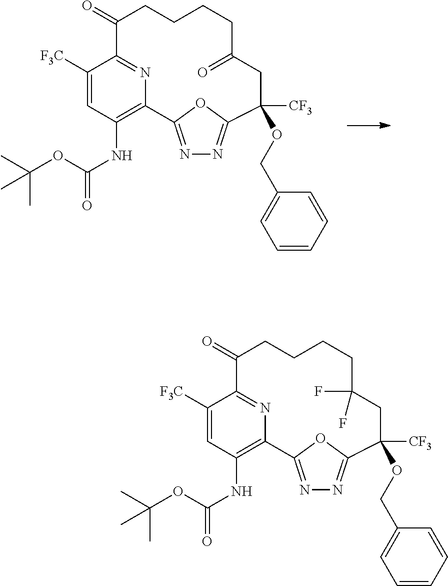 [SMILES: CC(C)(C)OC(=O)Nc1cc(C(F)(F)F)c2nc1C1=NN=C(O1)[C@@](OCc1ccccc1)(C(F)(F)F)CC(=O)CCCCC2=O ; CC(C)(C)OC(=O)Nc1cc(C(F)(F)F)c2nc1C1=NN=C(O1)[C@@](OCc1ccccc1)(C(F)(F)F)CC(F)(F)CCCCC2=O]

In a 20-mL microwave vial, tert-butyl N-[(6R)-6-benzyloxy-8,13-dioxo-6,15-bis(trifluoromethyl)-19-oxa-3,4,18-triazatricyclo[12.3.1.12,5]nonadeca-1(17),2,4,14(18),15-pentaen-17-yl]carbamate (90 mg, 0.1401 mmol) was dissolved in 1,2-dichloroethane (2.25 mL) at room temperature. To the stirring solution was slowly added diethylaminosulfur trifluoride (900 μL, 6.812 mmol) allowing the mixture to stir at room temperature for 15 additional minutes. Then, the mixture was capped and heated to 80° C. for 16 hours behind a blast shield. The resultant mixture was quenched slowly at 0° C. with saturated aqueous NaHCO₃ (20 mL). The mixture was diluted with dichloromethane and washed with water then dried over sodium sulfate, filtered and concentrated. The crude material was purified by reverse-phase preparative chromatography utilizing a C₁₈ column eluting with a gradient from 30% to 99% acetonitrile in 5 mM aqueous HCl to afford as a light yellow solid, tert-butyl N-[(6R)-6-benzyloxy-8,8-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (19 mg, 20%). ESI-MS m/z calc. 664.1932, found 665.2 (M+1)⁺; Retention time: 2.27 minutes (LC Method J).

### Step 5: tert-Butyl N-[(6R)-8,8-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate

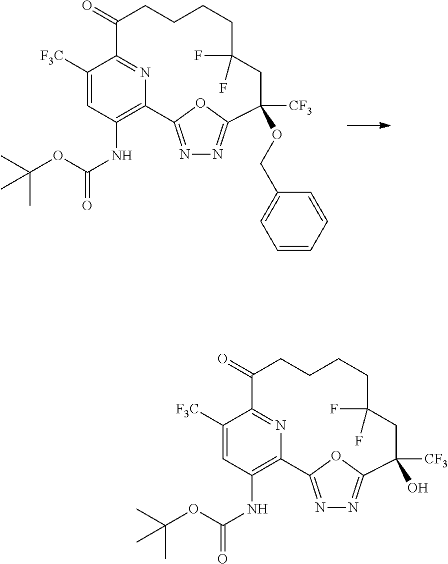 [SMILES: CC(C)(C)OC(=O)Nc1cc(C(F)(F)F)c2nc1C1=NN=C(O1)[C@@](OCc1ccccc1)(C(F)(F)F)CC(F)(F)CCCCC2=O ; CC(C)(C)OC(=O)Nc1cc(C(F)(F)F)c2nc1C1=NN=C(O1)[C@@](O)(C(F)(F)F)CC(F)(F)CCCCC2=O]

In a pressure reactor, a solution of tert-butyl N-[(6R)-6-benzyloxy-8,8-difluoro-13-oxo-6,15-bis(trifluoromethyl)-19-oxa-3,4,18-triazatricyclo[12.3.1.12,5]nonadeca-1(17),2,4,14(18),15-pentaen-17-yl]carbamate (56 mg, 0.08427 mmol) in acetic acid (1 mL) and ethyl acetate (1 mL) was purged with nitrogen. Then, 10% Pd/C (95 mg, 0.08927 mmol) was added. The mixture was degassed with nitrogen, then filled with hydrogen gas and stirred at 180 psi for 4 hours. The mixture was degassed with nitrogen, then filled with hydrogen gas and stirred at 180 psi for 2 additional hours. The reaction was filtered and then washed the celite plug with excess acetonitrile and ethyl acetate and concentrated the organic filtrate. The crude material was then purified by silica gel chromatography using a gradient from 100% hexanes to 60% ethyl acetate in hexanes then placed under high vac pump for 2 hours to afford as a white solid, tert-butyl N-[(6R)-8,8-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (28 mg, 58%). ¹H NMR (400 MHz, Chloroform-d) δ 9.94 (s, 1H), 9.43 (s, 1H), 5.99 (s, 1H), 3.56 (d, J=17.3 Hz, 1H), 3.16-3.07 (m, 2H), 2.86-2.71 (m, 1H), 2.66 (dd, J=18.7, 7.0 Hz, 1H), 2.26 (t, J=13.3 Hz, 1H), 2.08 (m, 1H), 1.90-1.77 (m, 2H), 1.56 (s, 9H), 1.45 (s, 1H) ppm. ESI-MS m/z calc. 574.14624, found 575.2 (M+1)⁺; Retention time: 1.77 minutes (LC Method J).

### Step 6: (6R)-17-Amino-8,8-difluoro-6-hydroxy-6,15-bis(trifluoromethyl)-19-oxa-3,4,18-triazatricyclo[12.3.1.12,5]nonadeca-1(18),2,4,14,16-pentaen-13-one (Compound 44)

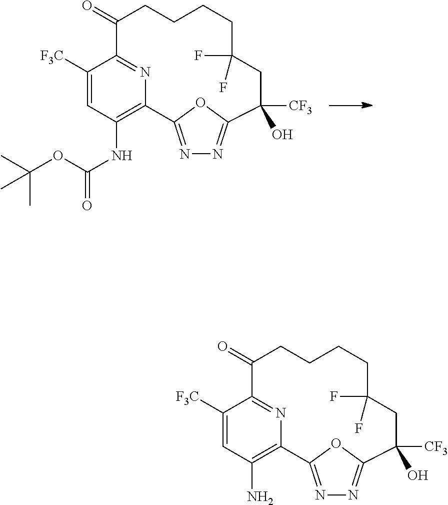 [SMILES: CC(C)(C)OC(=O)Nc1cc(C(F)(F)F)c2nc1C1=NN=C(O1)[C@@](O)(C(F)(F)F)CC(F)(F)CCCCC2=O ; Nc1cc(C(F)(F)F)c2nc1C1=NN=C(O1)[C@@](O)(C(F)(F)F)CC(F)(F)CCCCC2=O]

tert-Butyl N-[(6R)-8,8-difluoro-6-hydroxy-13-oxo-6,15-bis(trifluoromethyl)-19-oxa-3,4,18-triazatricyclo[12.3.1.12,5]nonadeca-1(17),2,4,14(18),15-pentaen-17-yl]carbamate (24 mg, 0.04178 mmol) was dissolved in dichloromethane (1 mL) and to the mixture was added TFA (200 μL, 2.596 mmol) and the mixture was then stirred at room temperature. After 3 hours, the mixture was evaporated to dryness, then diluted with ether and reconcentrated. The isolated residue was purified by reverse-phase preparative chromatography utilizing a C₁₈ column and a gradient from 1% to 99% acetonitrile in 5 mM aqueous HCl to produce as a white solid, (6R)-17-amino-8,8-difluoro-6-hydroxy-6,15-bis(trifluoromethyl)-19-oxa-3,4,18-triazatricyclo[12.3.1.12,5]nonadeca-1(18),2,4,14,16-pentaen-13-one (5.3 mg, 26%). ¹H NMR (400 MHz, Chloroform-d) δ 7.57 (s, 1H), 6.15 (s, 2H), 6.04 (s, 1H), 3.57 (d, J=17.2 Hz, 1H), 3.19-3.08 (m, 2H), 2.85-2.72 (m, 1H), 2.67 (dd, J=19.0, 7.2 Hz, 1H), 2.28 (t, J=13.2 Hz, 1H), 2.17-2.03 (m, 1H), 1.94-1.76 (m, 2H), 1.44 (q, J=6.6 Hz, 1H) ppm. ESI-MS m/z calc. 474.0938, found 475.2 (M+1)⁺; Retention time: 1.74 minutes (LC Method A).

### Example 23: Preparation of (6R)-17-amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (Compound 45)

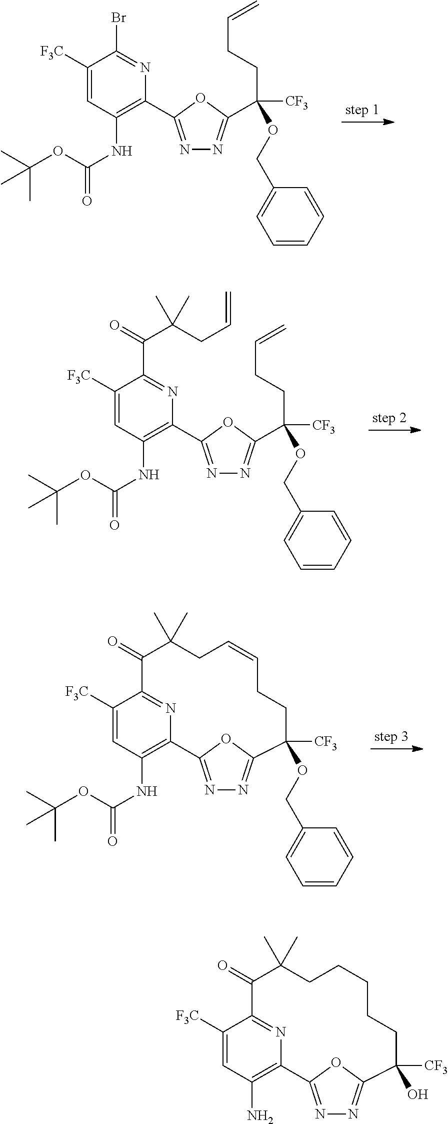 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(C(=O)C(C)(C)CC=C)c(C(F)(F)F)cc2NC(=O)OC(C)(C)C)o1)C(F)(F)F ; CC(C)(C)OC(=O)Nc1cc(C(F)(F)F)c2nc1C1=NN=C(O1)[C@@](OCc1ccccc1)(C(F)(F)F)CC/C=C\CC(C)(C)C2=O ; CC1(C)CCCCC[C@](O)(C(F)(F)F)C2=NN=C(O2)c2nc(c(C(F)(F)F)cc2N)C1=O]

### Step 1: tert-Butyl N-[2-[5-[(1R)-1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate

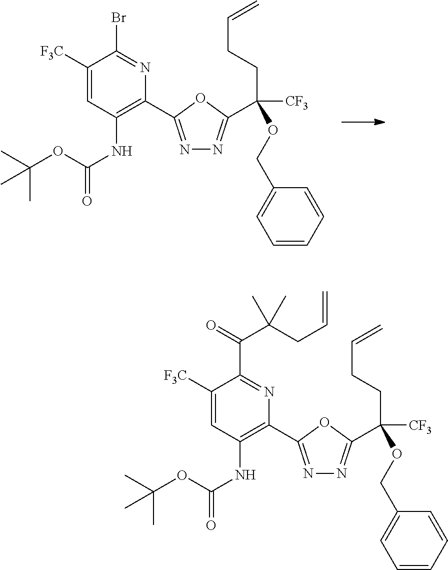 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(C(=O)C(C)(C)CC=C)c(C(F)(F)F)cc2NC(=O)OC(C)(C)C)o1)C(F)(F)F]

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (1.952 g, 2.997 mmol) in diethyl ether (30 mL) at −78° C. was added n-butyllithium (2.8 mL of 2.5 M in hexanes, 7 mmol) dropwise keeping the reaction temperature less than −70° C. The mixture was stirred at −78° C. for 15 minutes, then a solution of N-methoxy-N,2,2-trimethyl-pent-4-enamide (1.7 g, 9.928 mmol) in diethyl ether (3 mL) was added. The mixture was stirred at −78° C. for 10 minutes, then at 0° C. for 60 minutes. The reaction was then quenched with the addition of 25 mL of saturated aqueous NH₄Cl. The organic phase was separated and the aqueous phase was extracted with ethyl acetate then the combined organic phases were dried over MgSO₄, filtered and concentrated in vacuo. The crude product was purified by reverse phase C₁₈ chromatography eluting with a gradient from 50% to 100% acetonitrile in water giving tert-butyl N-[2-[5-[(1R)-1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate (1.0 g, 49%). ¹H NMR (400 MHz, DMSO-d₆) δ 10.18 (s, 1H), 9.15 (s, 1H), 7.39-7.28 (m, 5H), 5.91-5.67 (m, 2H), 5.13-5.05 (m, 1H), 5.04-4.96 (m, 3H), 4.82-4.57 (m, 2H), 2.60 (d, J=7.4 Hz, 2H), 2.44 (s, 1H), 2.37-2.22 (m, 3H), 1.54 (s, 9H), 1.25 (s, 6H) ppm; ¹⁹F NMR (376 MHz, DMSO-d₆) δ−58.27, −72.93 ppm. ESI-MS m/z calc. 682.259, found 683.4 (M+1)⁺; Retention time: 4.11 minutes (LC Method FF).

### Step 2: tert-Butyl N-[(6R,9Z)-6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate

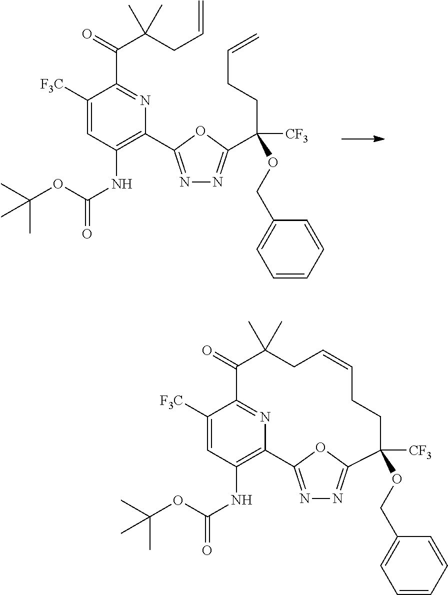

A solution of tert-butyl N-[2-[5-[(1R)-1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate (1.37 g, 2.007 mmol) in toluene (85 mL) was added dropwise over 20 min to a 120° C. preheated solution of [1,3-bis-(2-tolyl)-2-imidazolidinylidene]dichloro(2-isopropoxybenzylidene)ruthenium(II) (100 mg, 0.1753 mmol) in toluene (85 mL) which had a slow stream of nitrogen bubbled into it. With continued nitrogen bubbling, the mixture was stirred at 120° C. for 1 h, then additional catalyst, [1,3-bis-(2-tolyl)-2-imidazolidinylidene]dichloro(2-isopropoxybenzylidene)ruthenium(II) (35 mg, 0.06135 mmol) as a solution in toluene (1 mL) was added and the mixture stirred at 120° C. for 50 min. The reaction was quenched by cooling the mixture to around 55° C. and adding 2-sulfanylpyridine-3-carboxylic acid (145 mg, 0.9344 mmol) followed by Et₃N (140 μL, 1.004 mmol). The mixture was stirred overnight then filtered over celite and the solvent was removed in vacuo. The crude product was purified by reverse phase chromatography eluting with a gradient from 50% to 100% acetonitrile in water which afforded tert-butyl N-[(6R,9Z)-6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate (1 g, 76%). ¹H NMR (400 MHz, DMSO-d₆) δ 9.69 (d, J=45.8 Hz, 1H), 9.06 (d, J=13.4 Hz, 1H), 7.41-7.26 (m, 5H), 5.73 (m, J=17.8, 16.4, 7.2 Hz, 1H), 5.55 (m, J=28.2, 14.1, 6.9 Hz, 1H), 4.79-4.67 (m, 2H), 3.32 (s, 2H), 3.17-2.97 (m, 1H), 2.70-2.53 (m, 1H), 2.40-2.26 (m, 2H), 1.54 (d, J=3.3 Hz, 9H), 1.25-1.18 (m, 6H) ppm; ¹⁹F NMR (376 MHz, DMSO-d₆) δ−58.51, −58.93, −74.23, −74.31 ppm. ESI-MS m/z calc. 654.22766, found 655.4 (M+1)⁺; Retention time: 3.96 minutes (LC Method FF).

### Step 3: (6R)-17-Amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (Compound 45)

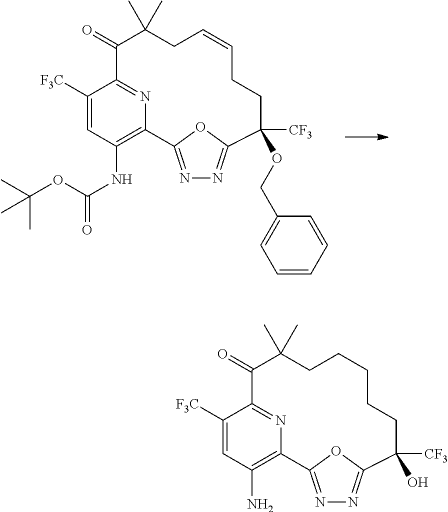 [SMILES: CC(C)(C)OC(=O)Nc1cc(C(F)(F)F)c2nc1C1=NN=C(O1)[C@@](OCc1ccccc1)(C(F)(F)F)CC/C=C\CC(C)(C)C2=O ; CC1(C)CCCCC[C@](O)(C(F)(F)F)C2=NN=C(O2)c2nc(c(C(F)(F)F)cc2N)C1=O]

A mixture of tert-butyl N-[(6R,9Z)-6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate (401 mg, 0.6126 mmol), 10% palladium on carbon (130 mg, 0.1222 mmol) and acetic acid (4 mL) was stirred at room temperature under 150 psi hydrogen for 13 h then the mixture was filtered and the filtrate evaporated. The residue was dissolved into ethyl acetate and washed with 1 M NaHCO₃, dried (MgSO₄), filtered and evaporated. The residue was dissolved into dichloromethane (4 mL) and Dess-Martin periodinane (26 mg, 0.0613 mmol) was added. The mixture was stirred at room temperature for 15 min then methanol (1 mL) was added and the mixture was diluted with dichloromethane and washed with 1 M Na₂S₂O₃ then 1 M NaHCO₃, dried (MgSO₄), filtered and evaporated. The residue was purified by silica gel chromatography using a gradient from 0% to 20% ethyl acetate in hexane to provide tert-butyl N-[(6R)-6-hydroxy-12,12-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (263 mg, 76%). ESI-MS m/z calc. 566.1964, found 567.2 (M+1)⁺; Retention time: 0.42 minutes. This material, tert-butyl N-[(6R)-6-hydroxy-12,12-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (210 mg, 0.37 mmol) was dissolved in TFA (2 mL) and water (100 μL) and stirred at room temperature for 15 min. The solvent was then evaporated and the residue was dissolved in ethyl acetate and washed with 1 M NaHCO₃, dried (MgSO₄), filtered and evaporated. The residue was purified by silica gel chromatography using a gradient from 5% to 50% ethyl acetate in hexane to provide (6R)-17-amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (166 mg, 58%). ¹H NMR (400 MHz, Chloroform-d) δ 7.45 (s, 1H), 5.84 (s, 2H), 4.02 (s, 1H), 2.49-2.39 (m, 1H), 2.39-2.29 (m, 1H), 2.21 (m, J=14.5, 10.0, 5.7 Hz, 1H), 1.85 (m, J=12.4, 4.5 Hz, 2H), 1.77-1.65 (m, 1H), 1.62-1.43 (m, 3H), 1.37-1.29 (m, 1H), 1.27 (s, 3H), 1.19 (s, 3H) ppm; ¹⁹F NMR (376 MHz, Chloroform-d) δ−60.21, −79.44 ppm. ESI-MS m/z calc. 466.14395, found 467.1 (M+1)⁺; Retention time: 1.54 minutes (LC Method Q).

### Example 24: Preparation of (6S)-17-amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (Compound 46)

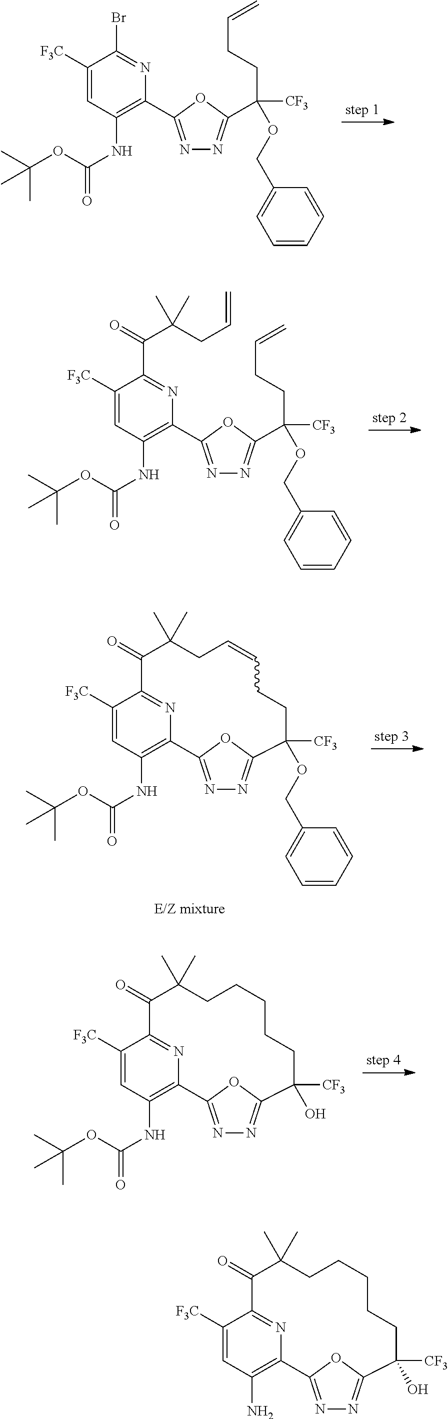 [SMILES: C=CCCC(OCc1ccccc1)(c1nnc(-c2nc(Br)c(C(F)(F)F)cc2NC(=O)OC(C)(C)C)o1)C(F)(F)F ; C=CCCC(OCc1ccccc1)(c1nnc(-c2nc(C(=O)C(C)(C)CC=C)c(C(F)(F)F)cc2NC(=O)OC(C)(C)C)o1)C(F)(F)F ; CC(C)(C)OC(=O)Nc1cc(C(F)(F)F)c2nc1C1=NN=C(O1)C(OCc1ccccc1)(C(F)(F)F)CCC=CCC(C)(C)C2=O ; CC(C)(C)OC(=O)Nc1cc(C(F)(F)F)c2nc1C1=NN=C(O1)C(O)(C(F)(F)F)CCCCCC(C)(C)C2=O ; CC1(C)CCCCC[C@@](O)(C(F)(F)F)C2=NN=C(O2)c2nc(c(C(F)(F)F)cc2N)C1=O]

### Step 1: tert-Butyl N-[2-[5-[1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate

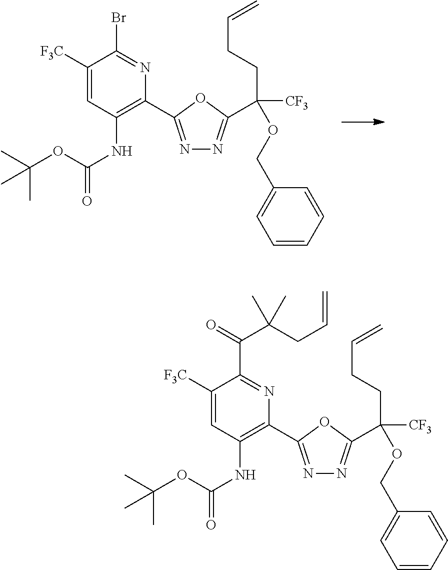 [SMILES: C=CCCC(OCc1ccccc1)(c1nnc(-c2nc(Br)c(C(F)(F)F)cc2NC(=O)OC(C)(C)C)o1)C(F)(F)F ; C=CCCC(OCc1ccccc1)(c1nnc(-c2nc(C(=O)C(C)(C)CC=C)c(C(F)(F)F)cc2NC(=O)OC(C)(C)C)o1)C(F)(F)F]

A solution of tert-butyl N-[2-[5-[1-benzyloxy-1-(trifluoromethyl)pent-4-enyl]-1,3,4-oxadiazol-2-yl]-6-bromo-5-(trifluoromethyl)-3-pyridyl]carbamate (415 mg, 0.6371 mmol) in diethyl ether (6 mL) was cooled to −78° C. in a dry ice/acetone bath. Then, n-BuLi (586 μL of 2.5 M, 1.465 mmol) in hexane was slowly added and the mixture was allowed to stir at this temperature for 15 min. Next, N-methoxy-N,2,2-trimethyl-pent-4-enamide (136 mg, 0.7942 mmol) was dissolved in diethyl ether (4 mL) and added to the reaction mixture via syringe. The mixture was stirred at this temperature for 15 min and then the dry ice bath was removed and the reaction was allowed to stir for an additional 10 min. The reaction was quenched by addition of 10 mL of dilute acetic acid (10%) and the organics were extracted with ethyl acetate (3×10 mL). The organic layers were combined, dried over Na₂SO₄, filtered, and the solvent was evaporated under vacuum. The residue was purified by C₁₈ reverse-phase chromatography using a gradient from 30% acetonitrile in water to 100% water which gave tert-butyl N-[2-[5-[1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate (119 mg, 27%). ¹H NMR (400 MHz, Chloroform-d) δ 10.22 (s, 1H), 9.36 (s, 1H), 7.59-6.65 (m, 5H), 5.97-5.48 (m, 2H), 5.21-4.97 (m, 4H), 4.84 (d, J=10.8 Hz, 1H), 4.68 (d, J=10.8 Hz, 1H), 2.72-2.57 (m, 2H), 2.55-2.44 (m, 2H), 2.42-2.35 (m, 1H), 2.32-2.19 (m, 1H), 1.58 (s, 9H), 1.30 (s, 6H) ppm. ESI-MS m/z calc. 682.259, found 683.3 (M+1)⁺; Retention time: 2.05 minutes (LC Method M).

### Step 2: tert-Butyl N-[6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture)

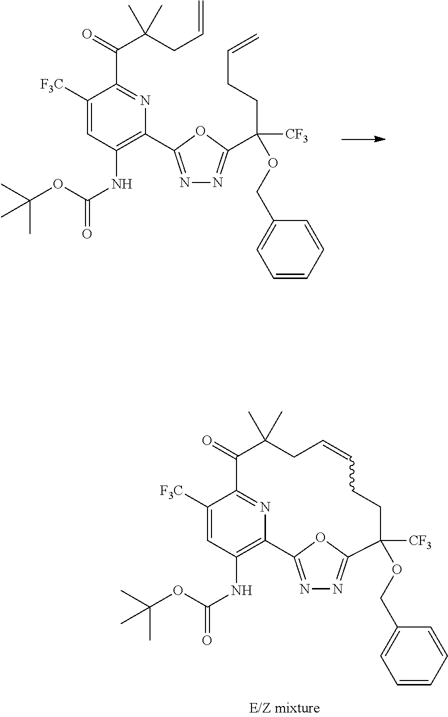 [SMILES: C=CCCC(OCc1ccccc1)(c1nnc(-c2nc(C(=O)C(C)(C)CC=C)c(C(F)(F)F)cc2NC(=O)OC(C)(C)C)o1)C(F)(F)F ; CC(C)(C)OC(=O)Nc1cc(C(F)(F)F)c2nc1C1=NN=C(O1)C(OCc1ccccc1)(C(F)(F)F)CCC=CCC(C)(C)C2=O]

A solution of [1,3-bis-(2-tolyl)-2-imidazolidinylidene]dichloro(2-isopropoxybenzylidene)ruthenium(II) (22.1 mg, 0.03874 mmol) in toluene (50 mL) was bubbled with nitrogen gas and heated to 70 to 75° C. Next, at this temperature, a solution of tert-butyl N-[2-[5-[1-benzyloxy-1-(trifluoromethyl)pent-4-enyl]-1,3,4-oxadiazol-2-yl]-6-(2,2-dimethylpent-4-enoyl)-5-(trifluoromethyl)-3-pyridyl]carbamate (220 mg, 0.3223 mmol) in toluene (30 mL) was added dropwise over 40 min while the temperature reaching was increased to 105° C. The mixture was then stirred 90 min at this temperature and every 30 mins, the solution was bubbled with nitrogen gas for 2 min. When all starting material was converted to the desired product, the reaction mixture was cooled to 40° C. and the solvent was evaporated under reduced pressure then the residue was purified by C₁₈ reverse-phase chromatography using a gradient from 30% acetonitrile in water to 100% water which gave tert-butyl N-[6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate (E/Z mixture) (160 mg, 76%). ESI-MS m/z calc. 654.22766, found 655.1 (M+1)⁺; Retention time: 2.09 minutes (LC Method M).

### Step 3: tert-Butyl N-[6-hydroxy-12,12-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate

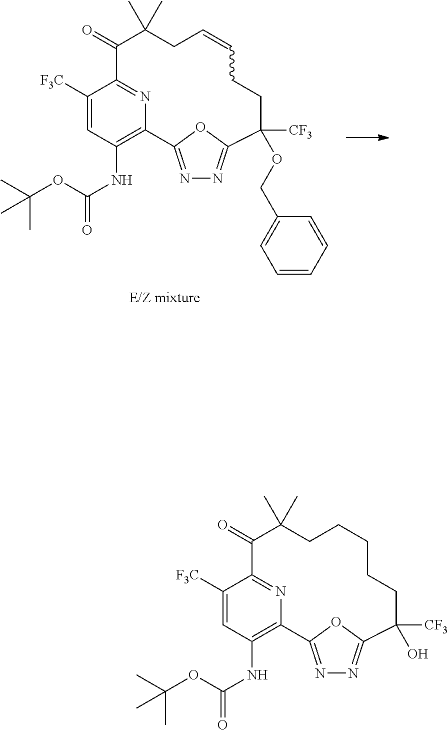 [SMILES: CC(C)(C)OC(=O)Nc1cc(C(F)(F)F)c2nc1C1=NN=C(O1)C(OCc1ccccc1)(C(F)(F)F)CCC=CCC(C)(C)C2=O ; CC(C)(C)OC(=O)Nc1cc(C(F)(F)F)c2nc1C1=NN=C(O1)C(O)(C(F)(F)F)CCCCCC(C)(C)C2=O]

To a solution of tert-butyl N-[6-benzyloxy-12,12-dimethyl-13-oxo-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture) (150 mg, 0.2291 mmol) in MeOH (2 mL) and acetic acid (2 mL) was added 10% Pd/C (49 mg, 0.02293 mmol, 50% wet) under nitrogen in a high pressure reactor. The reactor was sealed, evacuated and filled with nitrogen gas 3 times. The reactor was then evacuated and filled with hydrogen gas up to 120 psi. The mixture was stirred overnight then depressurized and filtered through a pad of celite washing with dichloromethane and MeOH. The residue was purified by C₁₈ reverse-phase chromatography using a gradient from 30% acetonitrile in water to 100% water which gave tert-butyl N-[6-hydroxy-12,12-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (46 mg, 35%). ESI-MS m/z calc. 566.1964, found 567.2 (M+1)⁺; Retention time: 1.96 minutes (LC Method J). The reaction also produced a significant amount of over-reduced product (M+2).

### Step 4: (6S)-17-Amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (Compound 46)

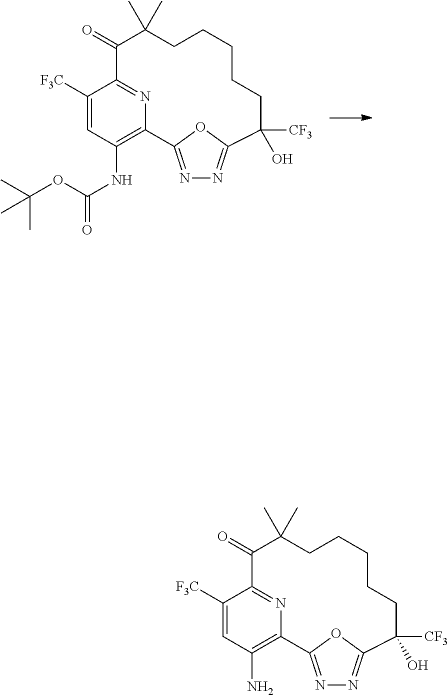 [SMILES: CC(C)(C)OC(=O)Nc1cc(C(F)(F)F)c2nc1C1=NN=C(O1)C(O)(C(F)(F)F)CCCCCC(C)(C)C2=O ; CC1(C)CCCCC[C@@](O)(C(F)(F)F)C2=NN=C(O2)c2nc(c(C(F)(F)F)cc2N)C1=O]

To a stirred solution of tert-butyl N-[6-hydroxy-12,12-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (80 mg, 0.1412 mmol) in dichloromethane (3 mL) was added TFA (2 mL, 25.96 mmol) and the mixture was stirred at room temperature for 35 minutes. The solvent was evaporated and the residue was purified by C₁₈ reverse-phase chromatography using a gradient from 30% acetonitrile in water to 90% water which gave the pure target racemic product. This racemic material was separated into single enantiomers using a normal phase SFC-MS method utilizing a ChiralPak IG column (250×21.2 mm, 5 m particle size) sold by Chiral Technologies eluting with 12% methanol (+20 mM NH₃) in CO₂ over 10 minutes. The second enantiomer to elute was isolated as (6S)-17-amino-6-hydroxy-12,12-dimethyl-6,15-bis(trifluoromethyl)-19-oxa-3,4,18-triazatricyclo[12.3.1.12,5]nonadeca-1(18),2,4,14,16-pentaen-13-one (15 mg, 23%). ¹H NMR (400 MHz, Chloroform-d) δ 7.45 (s, 1H), 5.85 (s, 2H), 4.16 (bs, 1H), 2.49-2.30 (m, 2H), 2.25-214 (m, 1H), 1.88-1.81 (m, 2H), 1.75-1.45 (m, 5H), 1.27 (s, 3H), 1.19 (s, 3H) ppm; ¹⁹F NMR (376 MHz, Chloroform-d) δ−60.21, −79.45 ppm; ESI-MS m/z calc. 466.14395, found 467.1 (M+1)⁺; Retention time: 1.33 minutes (LC Method J).

### Example 25: Preparation of (6R)-17-amino-6-hydroxy-11,11-dimethyl-6,15-bis(trifluoromethyl)-19-oxa-3,4,18-triazatricyclo[12.3.1.12,5]nonadeca-1(18),2,4,14,16-pentaen-13-one (Compound 47)

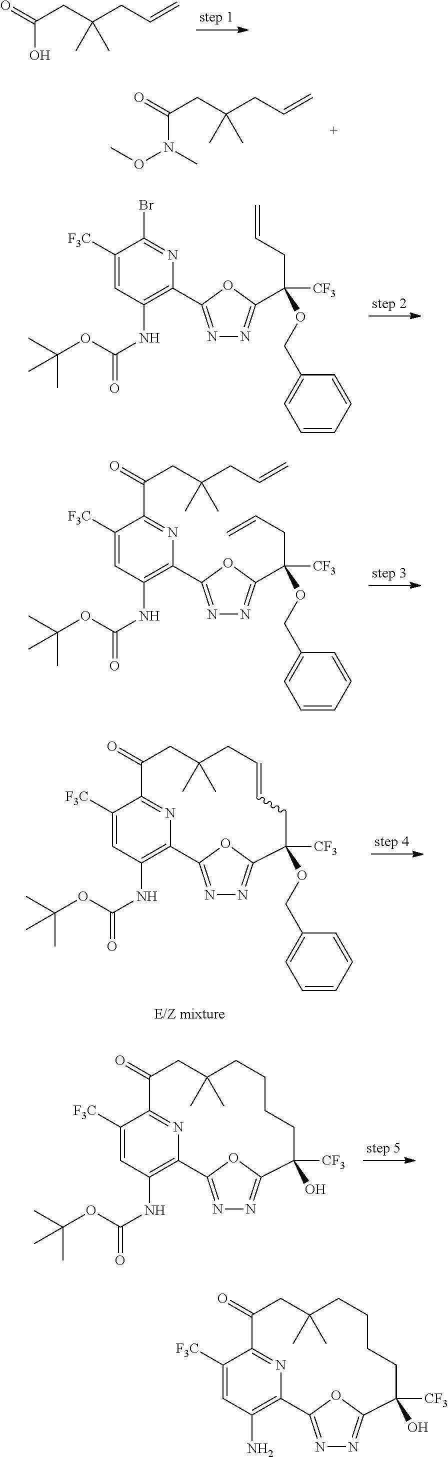

### Step 1: N-Methoxy-N,3,3-trimethyl-hex-5-enamide

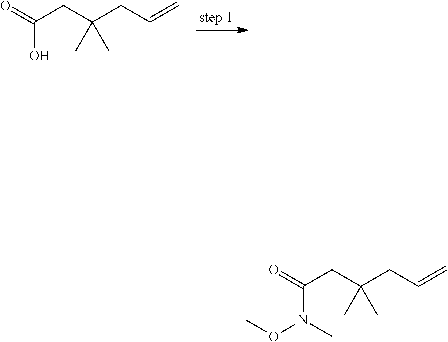

To a nitrogen purged solution of carbonyl diimidazole (7 g, 43.17 mmol) in dichloromethane (200 mL) at 0° C. was added 3,3-dimethylhex-5-enoic acid (5 g, 35.16 mmol) under nitrogen in an ice bath. Allowed to stir while warming to room temperature. After 16 h, added N-methoxymethanamine (hydrochloride salt) (8.75 g, 89.7 mmol). Then, the stirring mixture was cooled to 0° C. and pyridine (7.5 mL, 92.73 mmol) was slowly added and on completion of addition the mixture was stirred 5 minutes then the ice-water bath was removed and the solution was stirred overnight. The dichloromethane was removed by rotary evaporation, then added 1:1 diethyl ether/dichloromethane to the residue and extracted with saturated aqueous brine (1×). Dried the organic layer over sodium sulfate, filtered and concentrated to a tan oil which was purified by silica gel chromatography using a shallow gradient from 100% dichloromethane to 10% methanol in dichloromethane which provided as a colorless oil, N-methoxy-N,3,3-trimethyl-hex-5-enamide (5.6 g, 86%). ¹H NMR (400 MHz, Chloroform-d) δ 6.08-5.62 (m, 1H), 5.10-4.95 (m, 2H), 3.66 (s, 3H), 3.17 (s, 3H), 2.32 (s, 2H), 2.14 (dd, J=7.6, 1.4 Hz, 2H), 1.03 (s, 6H) ppm. ESI-MS m/z calc. 185.14159, found 186.2 (M+1)⁺; Retention time: 1.59 minutes (LC Method A).

### Step 2: tert-Butyl N-[2-[5-[(1R)-1-benzyloxy-1-(trifluoromethyl)but-3-enyl]-1,3,4-oxadiazol-2-yl]-6-(3,3-dimethylhex-5-enoyl)-5-(trifluoromethyl)-3-pyridyl]carbamate

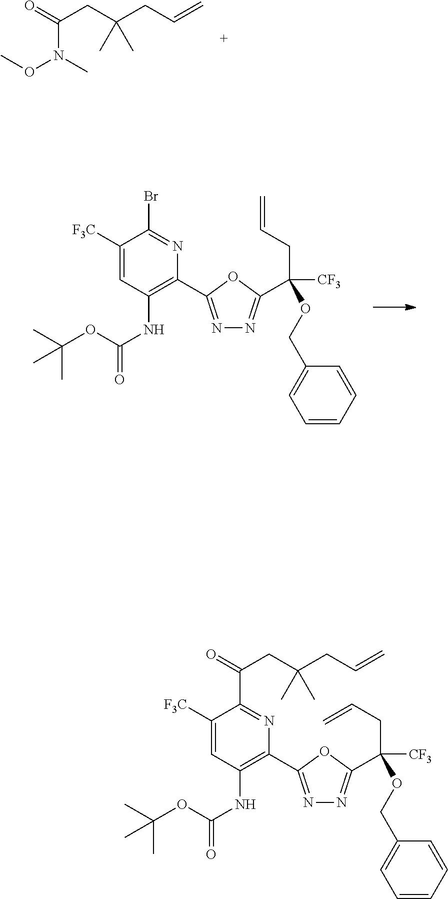 [SMILES: C=CCC(C)(C)CC(=O)N(C)OC ; C=CC[C@@](OCc1ccccc1)(c1nnc(-c2nc(Br)c(C(F)(F)F)cc2NC(=O)OC(C)(C)C)o1)C(F)(F)F ; C=CCC(C)(C)CC(=O)c1nc(-c2nnc([C@@](CC=C)(OCc3ccccc3)C(F)(F)F)o2)c(NC(=O)OC(C)(C)C)cc1C(F)(F)F]

To tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (300 mg, 0.4519 mmol) in ether (4 mL) at −78° C. was added n-BuLi (400 μL of 2.5 M, 1 mmol) as a solution in hexanes. After stirring for 15 min at −78° C., a solution of N-methoxy-N,3,3-trimethyl-hex-5-enamide (109 mg, 0.5884 mmol) in ether (1.4 mL) was added dropwise via cannula needle. The mixture was stirred at −78° C. for 23 min and then at 0° C. for 15 min. Then, the mixture was diluted with 1 M NH₄Cl in water (2 mL) the layers were separated. The organic layer was washed with 1 M NaHCO₃, dried (MgSO₄), filtered and evaporated. The residue was purified by silica gel chromatography using a gradient from 0% to 30% of a solution (10% ethyl acetate in hexanes) to hexanes over 15 min which provided tert-butyl N-[2-[5-[(1R)-1-benzyloxy-1-(trifluoromethyl)but-3-enyl]-1,3,4-oxadiazol-2-yl]-6-(3,3-dimethylhex-5-enoyl)-5-(trifluoromethyl)-3-pyridyl]carbamate (106 mg, 34%). ¹H NMR (400 MHz, Chloroform-d) δ 10.30 (s, 1H), 9.41 (s, 1H), 7.45-7.26 (m, 5H), 6.07-5.74 (m, 2H), 5.39-5.17 (m, 2H), 5.09-4.94 (m, 2H), 4.86 (d, J=10.8 Hz, 1H), 4.71 (d, J=10.9 Hz, 1H), 3.33-3.16 (m, 2H), 3.08 (d, J=2.0 Hz, 2H), 2.14 (m, J=7.5, 1.2 Hz, 2H), 1.58 (s, 9H), 1.04 (s, 6H) ppm; ¹⁹F NMR (376 MHz, Chloroform-d) δ−59.71, −73.14 ppm. ESI-MS m/z calc. 682.259, found 683.3 (M+1)⁺; Retention time: 0.7 minutes (LC Method T).

### Step 3: tert-Butyl N-[(6R)-6-benzyloxy-11,11-dimethyl-13-oxo-6,15-bis(trifluoromethyl)-19-oxa-3,4,18-triazatricyclo[12.3.1.12,5]nonadeca-1(18),2,4,8,14,16-hexaen-17-yl]carbamate (E/Z Mixture)

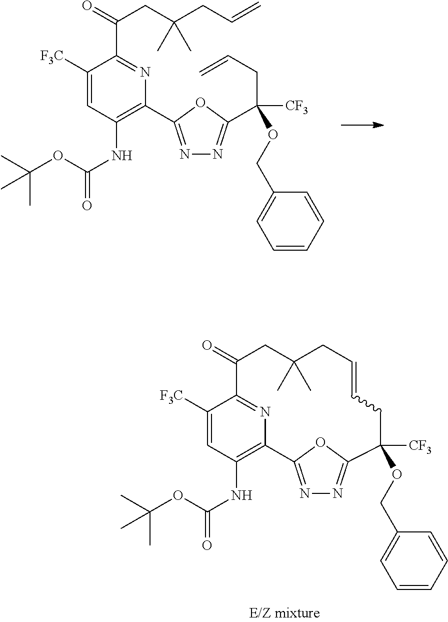 [SMILES: C=CCC(C)(C)CC(=O)c1nc(-c2nnc([C@@](CC=C)(OCc3ccccc3)C(F)(F)F)o2)c(NC(=O)OC(C)(C)C)cc1C(F)(F)F ; CC1(C)CC=CC[C@](OCc2ccccc2)(C(F)(F)F)C2=NN=C(O2)c2nc(c(C(F)(F)F)cc2NC(=O)OC(C)(C)C)C(=O)C1]

A solution of tert-butyl N-[2-[5-[(1R)-1-benzyloxy-1-(trifluoromethyl)but-3-enyl]-1,3,4-oxadiazol-2-yl]-6-(3,3-dimethylhex-5-enoyl)-5-(trifluoromethyl)-3-pyridyl]carbamate (407 mg, 0.5962 mmol) and benzylidene[1,3-bis(2,4,6-trimethylphenyl)-2-imidazolidinylidene]dichloro(tricyclohexylphosphine)ruthenium (76 mg, 0.08952 mmol) in toluene (60 mL) was heated at 120° C. for 30 min. Then, benzylidene[1,3-bis(2,4,6-trimethylphenyl)-2-imidazolidinylidene]dichloro(tricyclohexylphosphine)ruthenium (25 mg, 0.02945 mmol) was added and the solution was stirred at 120° C. for 50 min. Then, additional benzylidene[1,3-bis(2,4,6-trimethylphenyl)-2-imidazolidinylidene]dichloro(tricyclohexylphosphine)ruthenium (25 mg, 0.02945 mmol) was added and the solution was stirred at 120° C. for 30 min. Then, additional benzylidene[1,3-bis(2,4,6-trimethylphenyl)-2-imidazolidinylidene]dichloro(tricyclohexylphosphine)ruthenium (25 mg, 0.02945 mmol) was added and the solution was stirred at 120° C. for 30 min. Then, more benzylidene[1,3-bis(2,4,6-trimethylphenyl)-2-imidazolidinylidene]dichloro(tricyclohexylphosphine)ruthenium (25 mg, 0.02945 mmol) was added and the solution stirred at 120° C. for 30 min. Then, additional benzylidene[1,3-bis(2,4,6-trimethylphenyl)-2-imidazolidinylidene]dichloro(tricyclohexylphosphine)ruthenium (25 mg, 0.02945 mmol) was added and the solution stirred at 120° C. for 20 min. The solvent was evaporated and the residue was subjected to silica gel column chromatography using a gradient of 0% to 20% ethyl acetate in hexanes to provide partially purified material. This material was dissolved in 1 mL methanol and subjected to preparative HPLC using a Luna 75×30 mm C₁₈ column (5 m particle size) eluting with a gradient of 70% to 99% acetonitrile in 5 mM aqueous HCl which provided tert-butyl N-[(6R)-6-benzyloxy-11,11-dimethyl-13-oxo-6,15-bis(trifluoromethyl)-19-oxa-3,4,18-triazatricyclo[12.3.1.12,5]nonadeca-1(18),2,4,8,14,16-hexaen-17-yl]carbamate (E/Z Mixture) (21 mg, 5%). ESI-MS m/z calc. 654.22766, found 655.3 (M+1)⁺; Retention time: 0.62 minutes (LC Method T).

### Step 4: tert-butyl N-[(6R)-6-hydroxy-11,11-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate

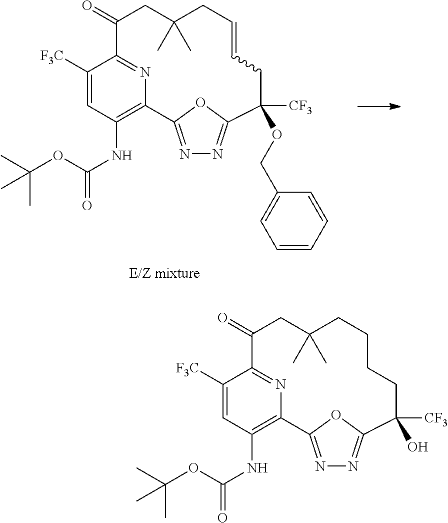 [SMILES: CC1(C)CC=CC[C@](OCc2ccccc2)(C(F)(F)F)C2=NN=C(O2)c2nc(c(C(F)(F)F)cc2NC(=O)OC(C)(C)C)C(=O)C1 ; CC1(C)CCCC[C@](O)(C(F)(F)F)C2=NN=C(O2)c2nc(c(C(F)(F)F)cc2NC(=O)OC(C)(C)C)C(=O)C1]

In a 250 mL round bottom flask, a solution of tert-butyl N-[(6R)-6-benzyloxy-11,11-dimethyl-13-oxo-6,15-bis(trifluoromethyl)-19-oxa-3,4,18-triazatricyclo[12.3.1.12,5]nonadeca-1(18),2,4,8,14,16-hexaen-17-yl]carbamate (E/Z mixture) (23 mg, 0.03514 mmol) in ethyl acetate (1 mL) was purged with nitrogen. Then, Pd/C (38 mg of 10% w/w, 0.03571 mmol) was added. The mixture was degassed with nitrogen, then filled with a balloon containing hydrogen gas and stirred at 1 atm of pressure for 4 hours. The mixture was filtered, washing the celite plug with excess ethyl acetate and then concentrated the filtrate. The crude material was purified by reverse-phase preparative chromatography utilizing a C₁₈ column and a gradient from 30% to 99% acetonitrile in 5 mM HCl to afford as an off-white foam, tert-butyl N-[(6R)-6-hydroxy-11,11-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (12.3 mg, 62%). ESI-MS m/z calc. 566.1964, found 567.2 (M+1)⁺; Retention time: 2.01 minutes (LC Method J).

### Step 5: (6R)-17-Amino-6-hydroxy-11,11-dimethyl-6,15-bis(trifluoromethyl)-19-oxa-3,4,18-triazatricyclo[12.3.1.12,5]nonadeca-1(18),2,4,14,16-pentaen-13-one (Compound 47)

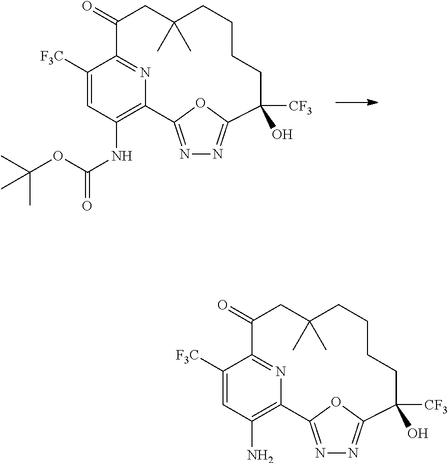 [SMILES: CC1(C)CCCC[C@](O)(C(F)(F)F)C2=NN=C(O2)c2nc(c(C(F)(F)F)cc2NC(=O)OC(C)(C)C)C(=O)C1 ; CC1(C)CCCC[C@](O)(C(F)(F)F)C2=NN=C(O2)c2nc(c(C(F)(F)F)cc2N)C(=O)C1]

tert-Butyl N-[(6R)-6-hydroxy-11,11-dimethyl-13-oxo-6,15-bis(trifluoromethyl)-19-oxa-3,4,18-triazatricyclo[12.3.1.12,5]nonadeca-1(18),2,4,14,16-pentaen-17-yl]carbamate (12.3 mg, 0.02171 mmol) was dissolved in dichloromethane (750 μL) and to the mixture was added TFA (100 μL, 1.298 mmol) and then stirred the reaction at room temperature for 45 minutes. The mixture was evaporated to dryness, then diluted with ether and reconcentrated. The isolated residue was purified by reverse-phase preparative chromatography utilizing a C₁₈ column eluting with 1% to 99% acetonitrile in 5 mM aqueous HCl to afford as a white solid, (6R)-17-amino-6-hydroxy-11,11-dimethyl-6,15-bis(trifluoromethyl)-19-oxa-3,4,18-triazatricyclo[12.3.1.12,5]nonadeca-1(18),2,4,14,16-pentaen-13-one (7.5 mg, 73%). ¹H NMR (400 MHz, Chloroform-d) δ 7.46 (s, 1H), 6.11 (s, 2H), 3.71 (d, J=1.5 Hz, 1H), 3.17 (d, J=12.0 Hz, 1H), 2.59 (d, J=12.0 Hz, 1H), 2.35 (dd, J=14.8, 8.0 Hz, 1H), 2.24 (d, J=15.0 Hz, 1H), 1.82-1.65 (m, 4H), 1.53-1.44 (m, 1H), 1.28 (dd, J=11.6, 6.7 Hz, 1H), 1.03 (d, J=11.1 Hz, 6H) ppm. ESI-MS m/z calc. 466.14395, found 467.2 (M+1)⁺; Retention time: 1.98 minutes (LC Method A).

### Example 26: Preparation of (6R)-17-amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclobutane]-13-one (Compound 48)

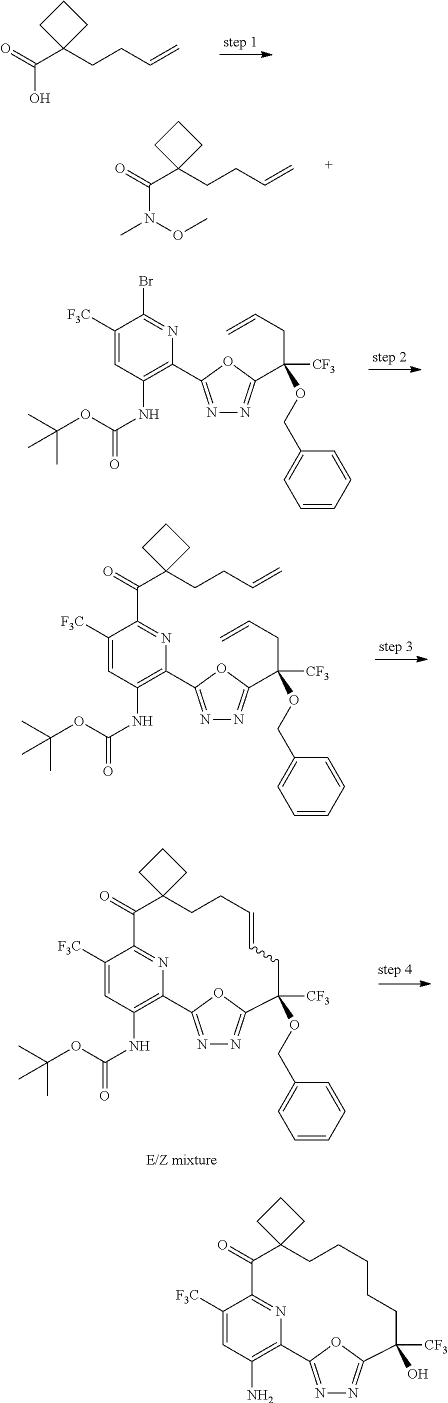 [SMILES: C=CCCC1(C(=O)O)CCC1 ; C=CCCC1(C(=O)N(C)OC)CCC1 ; C=CC[C@@](OCc1ccccc1)(c1nnc(-c2nc(Br)c(C(F)(F)F)cc2NC(=O)OC(C)(C)C)o1)C(F)(F)F ; C=CCCC1(C(=O)c2nc(-c3nnc([C@@](CC=C)(OCc4ccccc4)C(F)(F)F)o3)c(NC(=O)OC(C)(C)C)cc2C(F)(F)F)CCC1 ; CC(C)(C)OC(=O)Nc1cc(C(F)(F)F)c2nc1C1=NN=C(O1)[C@@](OCc1ccccc1)(C(F)(F)F)CC=CCCC1(CCC1)C2=O ; Nc1cc(C(F)(F)F)c2nc1C1=NN=C(O1)[C@@](O)(C(F)(F)F)CCCCCC1(CCC1)C2=O]

### Step 1: 1-But-3-enyl-N-methoxy-N-methyl-cyclobutanecarboxamide

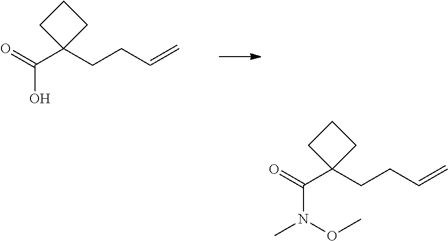

To a mixture of 1-but-3-enylcyclobutanecarboxylic acid (2.78 g, 16.23 mmol) in DCM (50 mL) at room temperature was slowly added solid carbonyl diimidazole (3.24 g, 19.98 mmol). The mixture was stirred at room temperature for 15 min and then N-methoxymethanamine (hydrochloride salt) (3.24 g, 33.22 mmol) was added and the mixture stirred at room temperature for 13 h. The slurry was diluted with 100 mL of water and 100 mL 1 M HCl, partitioned, and the organic layer was washed with brine then dried (MgSO₄), filtered and evaporated. The residue was purified by silica gel chromatography using a gradient from 5% to 75% ethyl acetate to hexane which provided 1-but-3-enyl-N-methoxy-N-methyl-cyclobutanecarboxamide (1.336 g, 42%). ¹H NMR (400 MHz, Chloroform-d) δ 5.93-5.66 (m, 1H), 5.02 (m, J=17.2, 1.6 Hz, 1H), 4.98-4.87 (m, 1H), 3.64 (s, 3H), 3.16 (s, 3H), 2.61-2.41 (m, 2H), 2.06-1.85 (m, 7H), 1.82-1.69 (m, 1H) ppm. ESI-MS m/z calc. 197.14159, found 198.2 (M+1)⁺; Retention time: 0.5 minutes (LC Method T).

### Step 2: tert-Butyl N-[2-[5-[(1R)-1-benzyloxy-1-(trifluoromethyl)but-3-enyl]-1,3,4-oxadiazol-2-yl]-6-(1-but-3-enylcyclobutanecarbonyl)-5-(trifluoromethyl)-3-pyridyl]carbamate

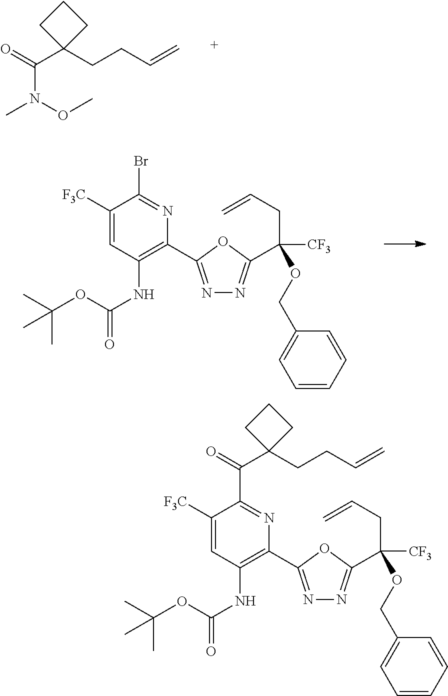

To tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (515 mg, 0.808 mmol) in ether (5 mL) at −78° C. was added n-BuLi (669 μL of 2.5 M, 1.672 mmol) as a solution in hexanes. After stirring a further 10 min at −78° C., a solution of 1-but-3-enyl-N-methoxy-N-methyl-cyclobutanecarboxamide (207 mg, 1.049 mmol) in ether (2.5 mL) was added dropwise via cannula needle. The mixture was stirred at −78° C. for 15 min and at 0° C. for 15 min. Then, the mixture was diluted with 1 M NH₄Cl in water (2 mL) and ether then partitioned. The organic layer was separated and washed with 1 M NaHCO₃, dried (MgSO₄), filtered and evaporated. The residue was purified by silica gel chromatography using a gradient from 5% to 20% of a solution (20% ethyl acetate in hexanes) to hexanes to provide tert-butyl N-[2-[5-[(1R)-1-benzyloxy-1-(trifluoromethyl)but-3-enyl]-1,3,4-oxadiazol-2-yl]-6-(1-but-3-enylcyclobutanecarbonyl)-5-(trifluoromethyl)-3-pyridyl]carbamate (127 mg, 21%). ESI-MS m/z calc. 694.259, found 695.4 (M+1)⁺; Retention time: 0.69 minutes (LC Method T).

### Step 3: tert-Butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclobutane]-17-yl]carbamate (E/Z Mixture)

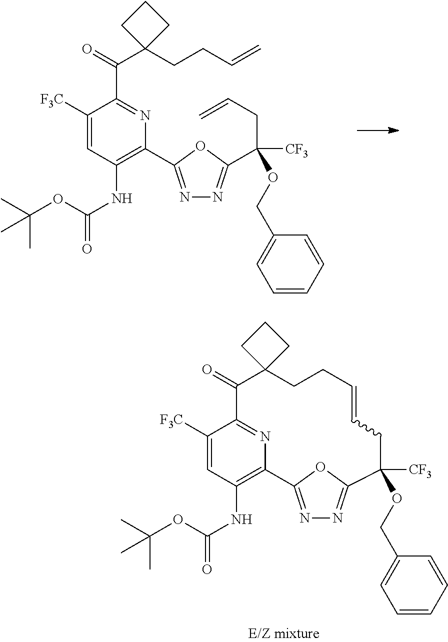

In a 100 mL, 3 neck flask, a solution of tert-butyl N-[2-[5-[(1R)-1-benzyloxy-1-(trifluoromethyl)but-3-enyl]-1,3,4-oxadiazol-2-yl]-6-(1-but-3-enylcyclobutanecarbonyl)-5-(trifluoromethyl)-3-pyridyl]carbamate (127 mg, 0.1737 mmol) and benzylidene[1,3-bis(2,4,6-trimethylphenyl)-2-imidazolidinylidene]dichloro(tricyclohexylphosphine)ruthenium (22 mg, 0.02591 mmol) in toluene (20 mL) with nitrogen constantly bubbling through the solution was heated at 120° C. for 30 min. Then, the solvent was evaporated. The residue was purified by silica gel chromatography using a gradient from 0% to 20% of a solution (20% ethyl acetate in hexanes) to hexanes to provide tert-butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclobutane]-17-yl]carbamate (E/Z Mixture) (77 mg, 66%). ESI-MS m/z calc. 666.22766, found 667.3 (M+1)⁺; Retention time: 0.63 minutes (LC Method T).

### Step 4: (6R)-17-Amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclobutane]-13-one (Compound 48)

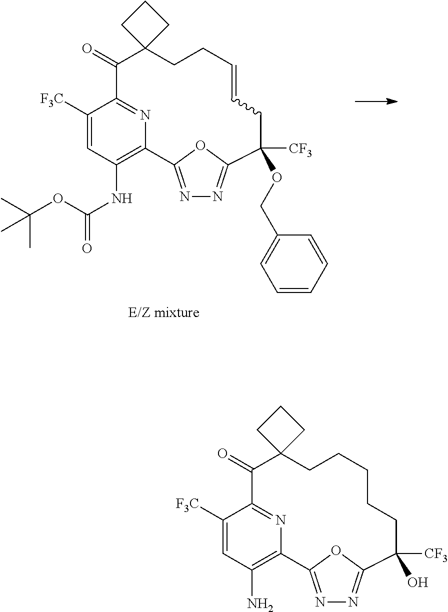 [SMILES: CC(C)(C)OC(=O)Nc1cc(C(F)(F)F)c2nc1C1=NN=C(O1)[C@@](OCc1ccccc1)(C(F)(F)F)CC=CCCC1(CCC1)C2=O ; Nc1cc(C(F)(F)F)c2nc1C1=NN=C(O1)[C@@](O)(C(F)(F)F)CCCCCC1(CCC1)C2=O]

Part 1: A mixture of tert-butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclobutane]-17-yl]carbamate (E/Z Mixture) (77 mg, 0.1155 mmol), 10% palladium on carbon (28 mg, 0.02631 mmol) and acetic acid (800 μL) was stirred at room temperature under 200 psi hydrogen gas for 13 h, then the mixture was filtered and the volatiles evaporated. The residue was dissolved in acetic acid (800 μL) was added to 10% palladium on carbon (22 mg, 0.02067 mmol) and stirred at room temperature under 200 psi hydrogen for 8 h, then the mixture was filtered and the filtrate evaporated. The residue was dissolved in ethyl acetate and washed with 1 M NaHCO₃, dried (MgSO₄), filtered and evaporated to provide 61 mg (95% pure, 87% yield) of the ketone, tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclobutane]-17-yl]carbamate which contained as a 3% impurity by UPLC the alcohol, tert-butyl N-[(6R)-6,13-dihydroxy-6,15-bis(trifluoromethyl)spiro[19-oxa-3,4,18-triazatricyclo[12.3.1.12,5]nonadeca-1(17),2,4,14(18),15-pentaene-12,1′-cyclobutane]-17-yl]carbamate.

Part 2: The 61 mg of 95% purity tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclobutane]-17-yl]carbamate was dissolved in DCM (1 mL) and Dess-Martin periodinane (9 mg, 0.02122 mmol) was added. The mixture was stirred at room temperature for 20 min then methanol (1 mL) was added and the mixture was diluted with DCM and washed with 1 M Na₂S203, 1 M NaHCO₃, then dried (MgSO₄), filtered and evaporated. The residue was purified by silica gel chromatography using a gradient from 0% to 20% ethyl acetate in hexane to provide tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclobutane]-17-yl]carbamate (54 mg, 77%). ESI-MS m/z calc. 578.1964, found 579.2 (M+1)⁺; Retention time: 0.44 minutes (LC Method T).

Part 3: A mixture of the product of part 2, TFA (500 μL) and water (25 μL) was stirred at room temperature for 20 min, then the solvent was evaporated. The residue was dissolved in ethyl acetate, washed with 1 M NaHCO₃, dried (MgSO₄), filtered and evaporated. The residue was purified by silica gel chromatography using a gradient from 5% to 40% ethyl acetate to hexanes which provided as a white solid, (6R)-17-amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclobutane]-13-one (32 mg, 58%). ¹H NMR (400 MHz, Chloroform-d) δ 7.50 (d, J=0.7 Hz, 1H), 5.97 (s, 2H), 3.63 (s, 1H), 2.58-2.33 (m, 5H), 2.30-2.15 (m, 2H), 2.13-2.01 (m, 1H), 1.96-1.72 (m, 6H), 1.69-1.57 (m, 1H), 1.48 (dd, J=13.7, 10.3 Hz, 1H) ppm; ¹⁹F NMR (376 MHz, Chloroform-d) δ−60.65, −79.76 ppm. ESI-MS m/z calc. 478.14395, found 479.2 (M+1)⁺; Retention time: 1.56 minutes (LC Method Q).

### Example 27: Preparation of (6R)-17-amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclopropane]-13-one (Compound 49)

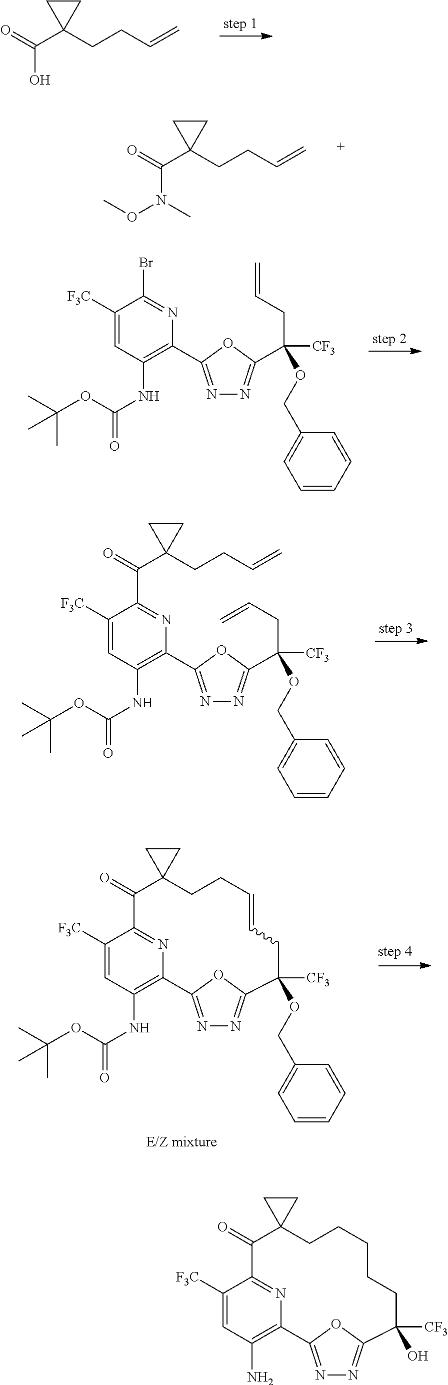

### Step 1: 1-But-3-enyl-N-methoxy-N-methyl-cyclopropanecarboxamide

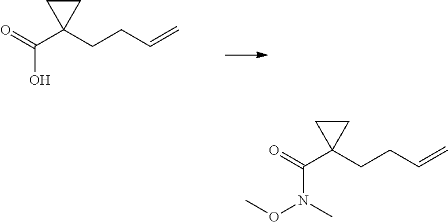

To a mixture of 1-but-3-enylcyclopropanecarboxylic acid (1.21 g, 8.459 mmol) in dichloromethane (25 mL) at room temperature was slowly added solid carbonyl diimidazole (1.69 g, 10.42 mmol). The mixture was stirred at room temperature for 15 min and then N-methoxymethanamine (hydrochloride salt) (1.69 g, 17.33 mmol) was added and the mixture was stirred at room temperature for 13 h. The slurry was concentrated by rotary evaporation, diluted with ether and 1 M HCl (50 mL, 50 mmol), partitioned, and the organic layer was then washed with water and brine then dried (MgSO₄), filtered and evaporated. The residue was purified silica gel chromatography using a gradient from 5% to 60% ethyl acetate in hexane which provided 1-but-3-enyl-N-methoxy-N-methyl-cyclopropanecarboxamide (970 mg, 63%). ¹H NMR (400 MHz, Chloroform-d) δ 5.82 (m, J=16.9, 10.2, 6.6 Hz, 1H), 5.06-4.98 (m, 1H), 4.96-4.82 (m, 1H), 3.72 (s, 3H), 3.23 (s, 3H), 2.26-2.12 (m, 2H), 1.76-1.63 (m, 2H), 1.08-0.94 (m, 2H), 0.63-0.53 (m, 2H) ppm. ESI-MS m/z calc. 183.12593, found 184.2 (M+1)⁺; Retention time: 0.41 minutes (LC Method T).

### Step 2: tert-Butyl N-[2-[5-[(1R)-1-benzyloxy-1-(trifluoromethyl)but-3-enyl]-1,3,4-oxadiazol-2-yl]-6-(1-but-3-enylcyclopropanecarbonyl)-5-(trifluoromethyl)-3-pyridyl]carbamate

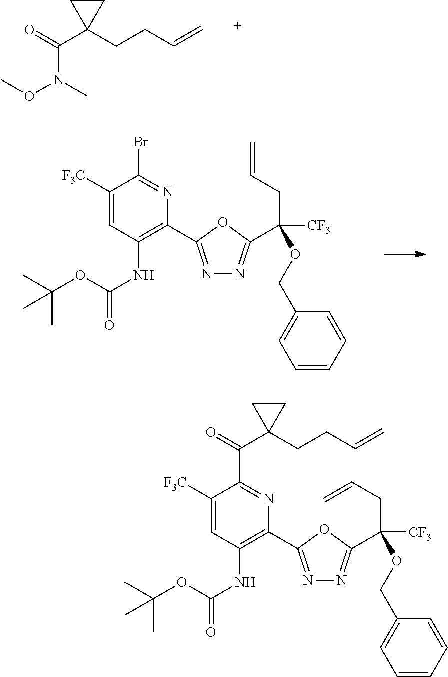

To tert-butyl N-[2-[5-[(1R)-1-benzyloxy-1-(trifluoromethyl)but-3-enyl]-1,3,4-oxadiazol-2-yl]-6-bromo-5-(trifluoromethyl)-3-pyridyl]carbamate (252 mg, 0.3954 mmol) in ether (3.8 mL) at −78° C. was added n-BuLi (330 μL of 2.5 M, 0.825 mmol) as a solution in hexanes. After stirring a further 10 min at −78° C., a solution of 1-but-3-enyl-N-methoxy-N-methyl-cyclopropanecarboxamide (145 mg, 0.7913 mmol) in ether (1.3 mL) was added dropwise via cannula needle. The mixture was stirred at −78° C. for 15 min and at 0° C. for 15 min. Then, the mixture was diluted with 1 M NH₄Cl in water (2 mL) and ether and partitioned. The organic layer was separated and washed with 1 M NaHCO₃, dried (MgSO₄), filtered and evaporated. The residue was purified silica gel chromatography using a gradient from 5% to 20% of a solution (20% ethyl acetate in hexanes) to hexanes to provide as a white solid, tert-butyl N-[2-[5-[(1R)-1-benzyloxy-1-(trifluoromethyl)but-3-enyl]-1,3,4-oxadiazol-2-yl]-6-(1-but-3-enylcyclopropanecarbonyl)-5-(trifluoromethyl)-3-pyridyl]carbamate (100 mg, 37%). ESI-MS m/z calc. 680.24335, found 681.3 (M+1)⁺; Retention time: 0.62 minutes (LC Method T).

### Step 3: tert-Butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclopropane]-17-yl]carbamate (E/Z Mixture)

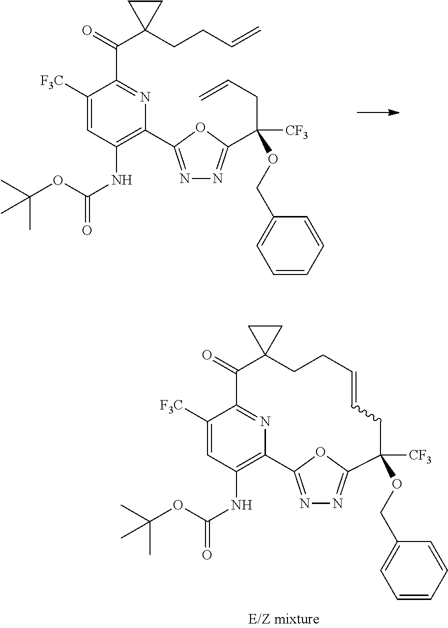 [SMILES: C=CCCC1(C(=O)c2nc(-c3nnc([C@@](CC=C)(OCc4ccccc4)C(F)(F)F)o3)c(NC(=O)OC(C)(C)C)cc2C(F)(F)F)CC1 ; CC(C)(C)OC(=O)Nc1cc(C(F)(F)F)c2nc1C1=NN=C(O1)[C@@](OCc1ccccc1)(C(F)(F)F)CC=CCCC1(CC1)C2=O]

In a 100 mL 3 neck flask, a solution of tert-butyl N-[2-[5-[(1R)-1-benzyloxy-1-(trifluoromethyl)but-3-enyl]-1,3,4-oxadiazol-2-yl]-6-(1-but-3-enylcyclopropanecarbonyl)-5-(trifluoromethyl)-3-pyridyl]carbamate (166 mg, 0.2439 mmol) and benzylidene[1,3-bis(2,4,6-trimethylphenyl)-2-imidazolidinylidene]dichloro(tricyclohexylphosphine)ruthenium (31 mg, 0.03651 mmol) in toluene (28 mL) with nitrogen gas constantly bubbling through the solution was heated at 120° C. for 30 min. Then, the solvent was evaporated. The residue was purified silica gel chromatography using a gradient from 0% to 30% of a solution (20% ethyl acetate in hexanes) to hexanes to provide tert-butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclopropane]-17-yl]carbamate (E/Z Mixture) (110 mg, 69%). ESI-MS m/z calc. 652.21204, found 653.4 (M+1)⁺; Retention time: 0.58 minutes (LC Method T).

### Step 4: (6R)-17-Amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclopropane]-13-one (Compound 49)

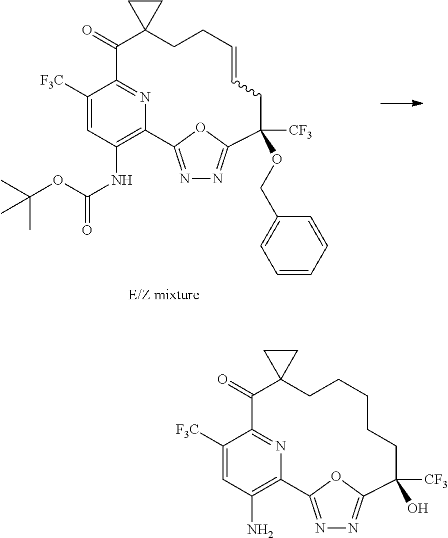 [SMILES: CC(C)(C)OC(=O)Nc1cc(C(F)(F)F)c2nc1C1=NN=C(O1)[C@@](OCc1ccccc1)(C(F)(F)F)CC=CCCC1(CC1)C2=O ; Nc1cc(C(F)(F)F)c2nc1C1=NN=C(O1)[C@@](O)(C(F)(F)F)CCCCCC1(CC1)C2=O]

Part 1: A mixture of tert-butyl N-[(6R)-6-benzyloxy-13-oxo-6,15-bis(trifluoromethyl)spiro[19-oxa-3,4,18-triazatricyclo[12.3.1.12,5]nonadeca-1(17),2,4,8,14(18),15-hexaene-12,1′-cyclopropane]-17-yl]carbamate (E/Z Mixture) (110 mg, 0.1686 mmol), 10% palladium on carbon (41 mg, 0.03853 mmol) and acetic acid (2.2 mL) was stirred at room temperature under 200 psi hydrogen gas for 16 h, then the mixture was filtered and the filtrate was evaporated. The residue was dissolved into ethyl acetate and washed with 1 M NaHCO₃, dried (MgSO₄), filtered and evaporated to provide 94 mg (90% pure, 89% yield) of the ketone, tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclopropane]-17-yl]carbamate which contained as a 1% impurity by UPLC the alcohol, tert-butyl N-[(6R)-6,13-dihydroxy-6,15-bis(trifluoromethyl)spiro[19-oxa-3,4,18-triazatricyclo[12.3.1.12,5]nonadeca-1(17),2,4,14(18),15-pentaene-12,1′-cyclopropane]-17-yl]carbamate.

Part 2: The 94 mg of 90% pure ketone, tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclopropane]-17-yl]carbamate obtained in part 1 was dissolved into dichloromethane (1.5 mL) and Dess-Martin periodinane (10 mg, 0.02358 mmol) was added. The mixture was stirred at room temperature for 20 min and then more Dess-Martin periodinane (10 mg, 0.02358 mmol) was added. The mixture was stirred at room temperature for 15 min and then methanol (1 mL) was added and the mixture was diluted with dichloromethane and washed with 1 M Na₂S₂O₃ followed by 1 M NaHCO₃ then dried (MgSO₄), filtered and evaporated. The residue was purified by silica gel chromatography using a gradient from 0% to 20% ethyl acetate in hexane to provide tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclopropane]-17-yl]carbamate (71 mg, 75%). ESI-MS m/z calc. 564.1807, found 565.2 (M+1)⁺; Retention time: 0.8 minutes (LC Method S).

Part 3: The material obtained in part 2, tert-butyl N-[(6R)-6-hydroxy-13-oxo-6,15-bis(trifluoromethyl)spiro[19-oxa-3,4,18-triazatricyclo[12.3.1.12,5]nonadeca-1(17),2,4,14(18),15-pentaene-12,1′-cyclopropane]-17-yl]carbamate (71 mg, 0.13 mmol) was dissolved in TFA (1.5 mL) and water (75 μL) and stirred at room temperature for 20 min. The solvent was evaporated and the residue was dissolved into ethyl acetate and washed with 1 M NaHCO₃, dried (MgSO₄), filtered and evaporated. The residue was purified by silica gel chromatography using a gradient from 5% to 40% ethyl acetate in hexane to provide (6R)-17-amino-6-hydroxy-6,15-bis(trifluoromethyl)spiro[19-oxa-3,4,18-triazatricyclo[12.3.1.12,5]nonadeca-1(18),2,4,14,16-pentaene-12,1′-cyclopropane]-13-one (51.5 mg, 87%). ¹H NMR (400 MHz, methanol-d4) δ 7.80-7.64 (m, 1H), 2.36 (m, J=13.6, 7.5, 5.7 Hz, 1H), 2.29-2.12 (m, 2H), 2.07-1.94 (m, 1H), 1.84-1.72 (m, 1H), 1.72-1.60 (m, 2H), 1.48-1.37 (m, 2H), 1.35-1.19 (m, 2H), 1.12 (m, J=11.6, 5.9, 5.2 Hz, 1H), 0.92 (m, J=9.0, 6.5, 3.5 Hz, 1H), 0.84 (m, J=9.2, 6.6, 3.1 Hz, 1H) ppm; ¹⁹F NMR (376 MHz, methanol-d4) δ−61.40, −80.93 ppm. ESI-MS m/z calc. 464.1283, found 465.2 (M+1)⁺; Retention time: 1.46 minutes (LC Method Q).

### Example 28: Preparation of (6R)-17-amino-12,12-dimethyl-6,15-bis(trifluoromethyl)-19-oxa-3,4,18-triazatricyclo[12.3.1.12,5]nonadeca-1(18),2,4,14,16-pentaen-6-ol (Compound 50)

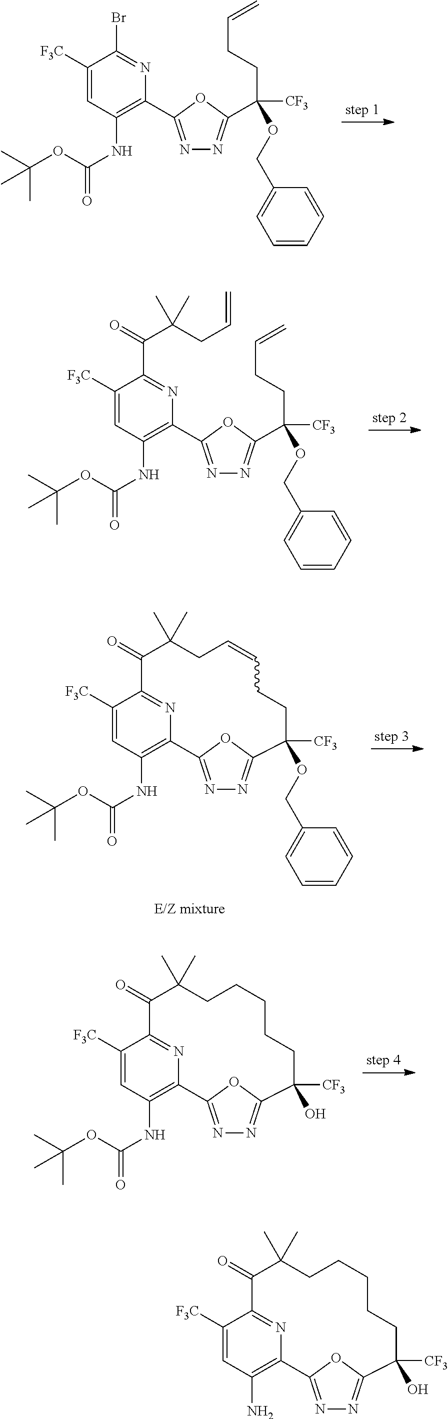 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(C(=O)C(C)(C)CC=C)c(C(F)(F)F)cc2NC(=O)OC(C)(C)C)o1)C(F)(F)F ; CC(C)(C)OC(=O)Nc1cc(C(F)(F)F)c2nc1C1=NN=C(O1)[C@@](OCc1ccccc1)(C(F)(F)F)CCC=CCC(C)(C)C2=O ; CC(C)(C)OC(=O)Nc1cc(C(F)(F)F)c2nc1C1=NN=C(O1)[C@@](O)(C(F)(F)F)CCCCCC(C)(C)C2=O ; CC1(C)CCCCC[C@](O)(C(F)(F)F)C2=NN=C(O2)c2nc(c(C(F)(F)F)cc2N)C1=O]

### Step 1: tert-Butyl N-[2-[5-[(1R)-1-benzyloxy-1-(trifluoromethyl)pent-4-enyl]-1,3,4-oxadiazol-2-yl]-6-(2,2-dimethylpent-4-enyl)-5-(trifluoromethyl)-3-pyridyl]carbamate

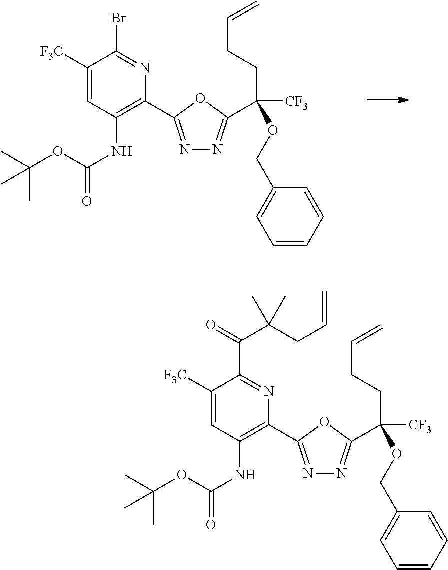 [SMILES: C=CCC[C@@](OCc1ccccc1)(c1nnc(-c2nc(Br)c(C(F)(F)F)cc2NC(=O)OC(C)(C)C)o1)C(F)(F)F ; C=CCC[C@@](OCc1ccccc1)(c1nnc(-c2nc(C(=O)C(C)(C)CC=C)c(C(F)(F)F)cc2NC(=O)OC(C)(C)C)o1)C(F)(F)F]

A dried screw-cap test tube, which was equipped with a magnetic stir bar and fitted with a Teflon septum, was charged with tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (500 mg, 0.7676 mmol), palladium acetate (9 mg, 0.0401 mmol), 2-dicyclohexylphosphino-2′,6′-bis(N,N-dimethylamino)biphenyl (34 mg, 0.0779 mmol) and dry THF (0.5 mL). The vessel was evacuated and backfilled with nitrogen (this process was repeated a total of 3 times). The solution was cooled to 0° C. in an ice bath and a solution of bromo(2,2-dimethylpent-4-enyl)zinc (4.6 mL of 0.5 M in THF, 2.3 mmol) was added. The ice-bath was removed and the reaction was stirred at room temperature overnight. The reaction mixture was then quenched by adding saturated aqueous ammonium chloride solution (5 mL) at 0° C. and extracted with ethyl acetate (40 mL). The organic layer was washed with brine (5 mL), dried over anhydrous sodium sulfate, filtered and concentrated under reduced pressure. The residue was purified by silica gel column chromatography using a gradient from 0% to 10% of ethyl acetate in heptanes to afford tert-butyl N-[2-[5-[(1R)-1-benzyloxy-1-(trifluoromethyl)pent-4-enyl]-1,3,4-oxadiazol-2-yl]-6-(2,2-dimethylpent-4-enyl)-5-(trifluoromethyl)-3-pyridyl]carbamate (499 mg, 95%) as a colorless oil. ¹H NMR (400 MHz, CDCl₃) δ 10.04 (s, 1H), 9.20 (s, 1H), 7.44-7.28 (m, 5H), 6.03-5.88 (m, 1H), 5.78 (m, 1H), 5.12-4.94 (m, 4H), 4.86 (d, J=10.8 Hz, 1H), 4.72 (d, J=10.8 Hz, 1H), 2.88 (s, 2H), 2.60-2.15 (m, 6H), 1.58 (s, 9H), 1.00 (d, J=2.9 Hz, 6H) ppm. ¹⁹F NMR (377 MHz, CDCl₃) δ−59.75 (s, 3F), −72.85 (s, 3F) ppm. ESI-MS m/z calc. 668.2797, found 669.3 (M+1)⁺; Retention time: 5.66 minutes (LC Method BB).

### Step 2: tert-Butyl N-[(6R)-6-benzyloxy-12,12-dimethyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture)

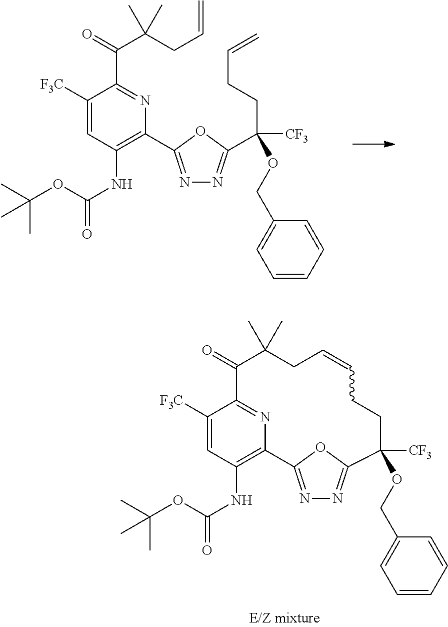

In a 500 mL three-necked flask equipped with a nitrogen inlet and a reflux condenser, a solution of tert-butyl N-[2-[5-[(1R)-1-benzyloxy-1-(trifluoromethyl)pent-4-enyl]-1,3,4-oxadiazol-2-yl]-6-(2,2-dimethylpent-4-enyl)-5-(trifluoromethyl)-3-pyridyl]carbamate (499 mg, 0.7269 mmol) in 1,2-dichloroethane (250 mL) was degassed by bubbling through the solution with nitrogen gas for 18 to 19 hours. A first portion of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (33 mg, 0.045 mmol) was added then the temperature was increase to 60° C. After 35 minutes, a second portion of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (32 mg, 0.0436 mmol) was added and the reaction was left to stir at 60° C. After a total reaction time of 2.5 hours, the flask was cooled to room temperature and the catalyst was quenched with DMSO (8 drops) and the reaction was concentrated under reduced pressure. The residue was purified by silica gel column chromatography using a gradient from 0% to 10% of ethyl acetate in heptanes to afford tert-butyl N-[(6R)-6-benzyloxy-12,12-dimethyl-6,15-bis(trifluoromethyl)-19-oxa-3,4,18-triazatricyclo[12.3.1.12,5]nonadeca-1(18),2,4,9,14,16-hexaen-17-yl]carbamate (E/Z mixture) (339 mg, 71%) as a white foamy solid. ¹H NMR (400 MHz, CDCl₃) δ 9.42 (s, 1H), 9.13 (s, 1H), 7.43-7.17 (m, 5H), 5.79-5.50 (m, 2H), 4.99-4.80 (m, 2H), 3.07 (dd, J=14.8, 7.7 Hz, 1H), 2.91 (s, 2H), 2.80-2.60 (m, 2H), 2.59-2.36 (m, 2H), 2.29-2.16 (m, 1H), 1.57 (s, 9H), 1.04 (d, J=4.9 Hz, 6H) ppm. ¹⁹F NMR (377 MHz, CDCl₃) δ−61.32 (s, 3F), −74.63 (s, 3F) ppm. ESI-MS m/z calc. 640.2484, found 641.3 (M+1)⁺; Retention time: 5.35 minutes (LC Method BB).

### Step 3: tert-Butyl N-[(6R)-6-hydroxy-12,12-dimethyl-6,15-bis(trifluoromethyl)-19-oxa-3,4,18-triazatricyclo[12.3.1.12,5]nonadeca-1(18),2,4,14,16-pentaen-17-yl]carbamate

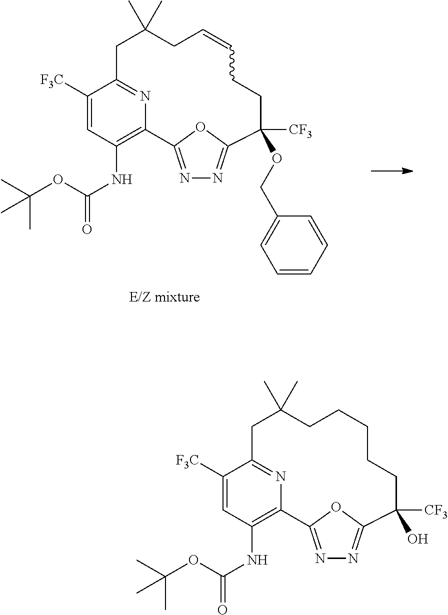 [SMILES: CC1(C)CC=CCC[C@](OCc2ccccc2)(C(F)(F)F)C2=NN=C(O2)c2nc(c(C(F)(F)F)cc2NC(=O)OC(C)(C)C)C1 ; CC1(C)CCCCC[C@](O)(C(F)(F)F)C2=NN=C(O2)c2nc(c(C(F)(F)F)cc2NC(=O)OC(C)(C)C)C1]

A solution of tert-butyl N-[(6R)-6-benzyloxy-12,12-dimethyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture) (339 mg, 0.5186 mmol) in methanol (10 mL) was purged three times (vacuum then nitrogen atmosphere). Added palladium on carbon (177 mg, 10% w/w, 0.1663 mmol, 50% wet) and purged twice under hydrogen atmosphere before leaving the reaction to stir under one atmosphere of hydrogen overnight. Purged once again under nitrogen atmosphere, then the reaction mixture was filtered over a short pad of celite and washed with methanol. Concentrated the filtrate under reduced pressure and transferred to a smaller flask with DCM. After concentrated some trace of Pd/C was observed. The product was diluted in DCM and filtered with a nylon 0.45 micron filter (on a syringe), concentrated and dried overnight under high vacuum to afford tert-butyl N-[(6R)-6-hydroxy-12,12-dimethyl-6,15-bis(trifluoromethyl)-19-oxa-3,4,18-triazatricyclo[12.3.1.12,5]nonadeca-1(18),2,4,14,16-pentaen-17-yl]carbamate (275 mg, 96%) as a white solid. ¹H NMR (400 MHz, CDCl₃) δ 9.21 (s, 1H), 9.11 (s, 1H), 3.60 (br s, 1H), 2.96-2.73 (m, 2H), 2.39 (m, 1H), 2.31-2.16 (m, 1H), 1.99-1.85 (m, 1H), 1.84-1.67 (m, 2H), 1.57 (s, 9H), 1.50 (m, 1H), 1.47-1.33 (m, 3H), 1.16 (m, 1H), 1.02 (s, 3H), 0.78 (s, 3H) ppm. ¹⁹F NMR (377 MHz, CDCl₃) δ−59.63 (s, 3F), −79.76 (s, 3F) ppm. ESI-MS m/z calc. 552.2171, found 553.2 (M+1)⁺; Retention time: 2.8 minutes (LC Method E). This crude material was used in the following step without further purification.

### Step 4: (6R)-17-Amino-12,12-dimethyl-6,15-bis(trifluoromethyl)-19-oxa-3,4,18-triazatricyclo[12.3.1.12,5]nonadeca-1(18),2,4,14,16-pentaen-6-ol (Compound 50)

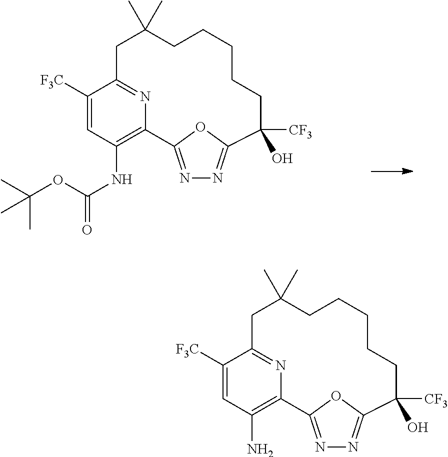 [SMILES: CC1(C)CCCCC[C@](O)(C(F)(F)F)C2=NN=C(O2)c2nc(c(C(F)(F)F)cc2NC(=O)OC(C)(C)C)C1 ; CC1(C)CCCCC[C@](O)(C(F)(F)F)C2=NN=C(O2)c2nc(c(C(F)(F)F)cc2N)C1]

To a solution of tert-butyl N-[(6R)-6-hydroxy-12,12-dimethyl-6,15-bis(trifluoromethyl)-19-oxa-3,4,18-triazatricyclo[12.3.1.12,5]nonadeca-1(18),2,4,14,16-pentaen-17-yl]carbamate (275 mg, 0.4977 mmol) in dichloromethane (4 mL) was added trifluoroacetic acid (5.92 g, 4 mL, 51.919 mmol). The mixture was stirred at room temperature for 2 hours. The mixture was then diluted with dichloromethane (10 mL), then concentrated by evaporation under reduced pressure. The residue was dissolved in ethyl acetate (50 mL), washed with aqueous saturated solution of sodium bicarbonate (3×10 mL) and brine (1×15 mL) then dried with anhydrous sodium sulphate, filtered and concentrated by evaporation under reduced pressure. The residue was purified by silica gel column chromatography using a gradient from 0% to 20% of ethyl acetate in heptanes. The product was dissolved in minimum of acetonitrile and water and freeze-dried overnight to afford (6R)-17-amino-12,12-dimethyl-6,15-bis(trifluoromethyl)-19-oxa-3,4,18-triazatricyclo[12.3.1.12,5]nonadeca-1(18),2,4,14,16-pentaen-6-ol (149 mg, 66%) as a white solid. ¹H NMR (400 MHz, CDCl₃) δ 7.38 (s, 1H), 5.45 (br. s., 2H), 3.60 (s, 1H), 2.88-2.64 (m, 2H), 2.46-2.32 (m, 1H), 2.30-2.14 (m, 1H), 1.99-1.85 (m, 1H), 1.84-1.66 (m, 2H), 1.63-1.48 (m, 2H), 1.47-1.35 (m, 2H), 1.23-1.10 (m, 1H), 1.01 (s, 3H), 0.77 (s, 3H) ppm. ¹⁹F NMR (377 MHz, CDCl₃) δ−59.94 (s, 3F), −79 ppm.

### Example 29: Preparation of 17-amino-11-benzyl-6-hydroxy-6,15-bis(trifluoromethyl)-19-oxa-3,4,11,18-tetrazatricyclo[12.3.1.12,5]nonadeca-1(18),2,4,14,16-pentaen-12-one (Compound 51)

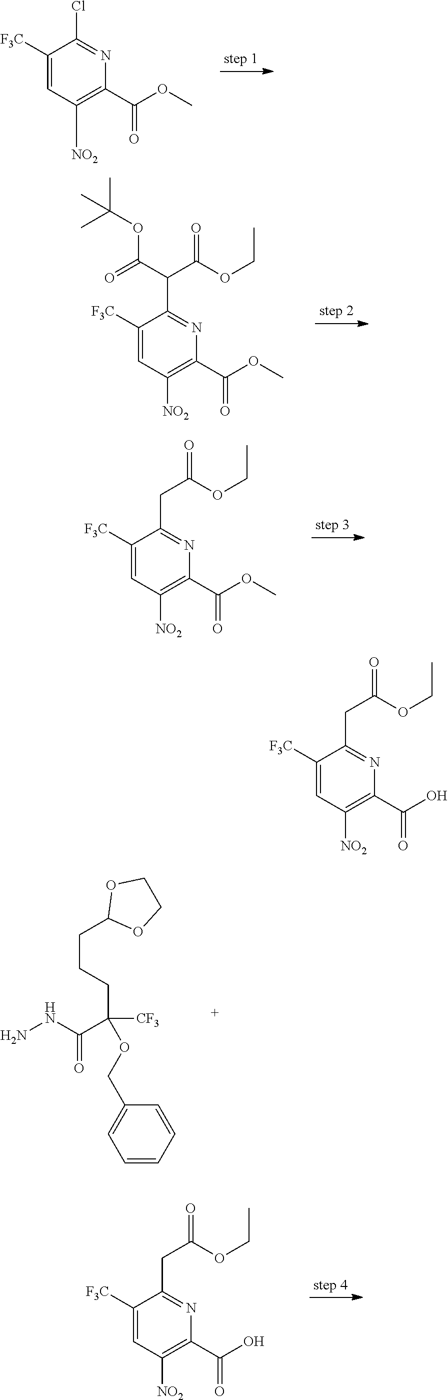

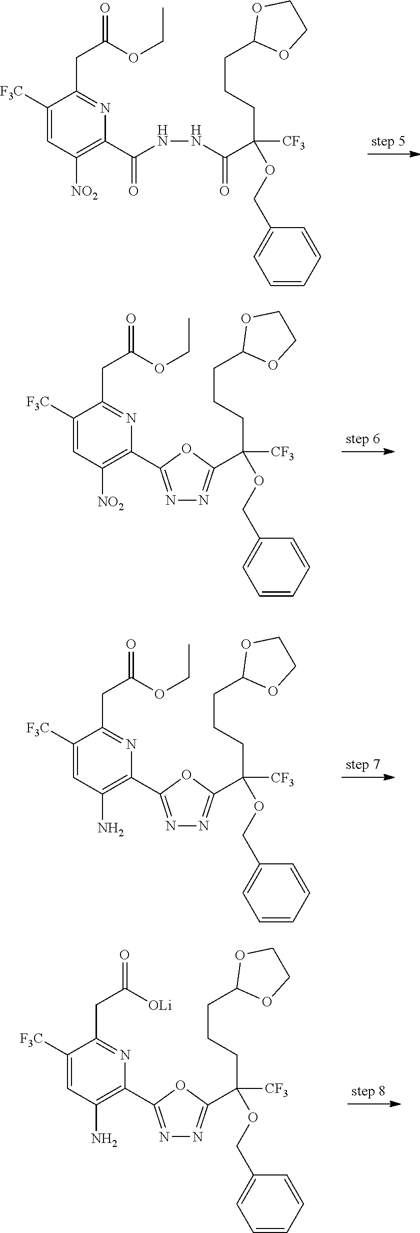

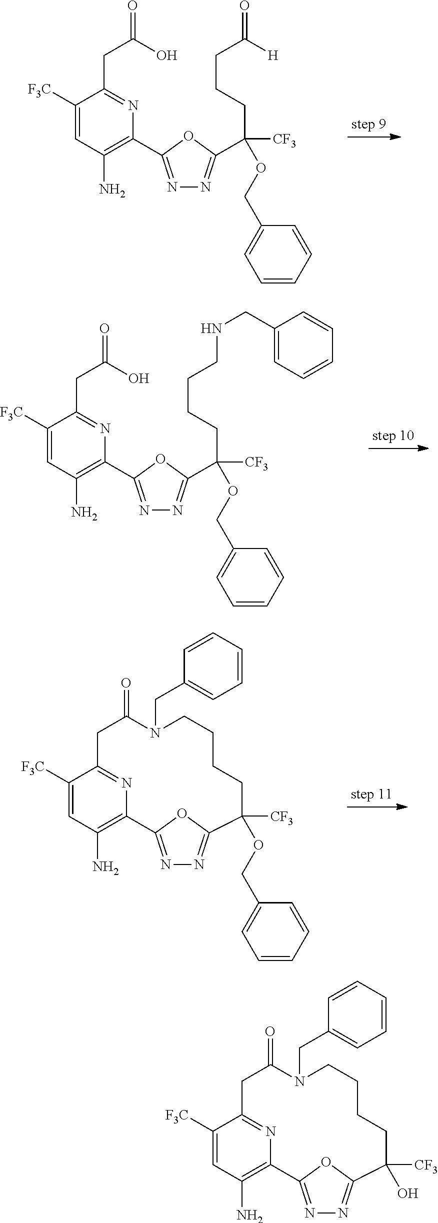

### Step 1: 01-tert-butyl 03-ethyl 2-[6-methoxycarbonyl-5-nitro-3-(trifluoromethyl)-2-pyridyl]propanedioate

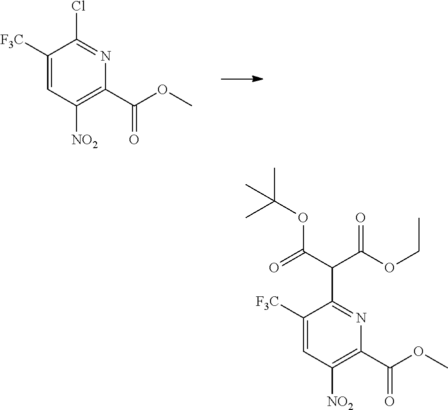 [SMILES: COC(=O)c1nc(Cl)c(C(F)(F)F)cc1[N+](=O)[O-] ; CCOC(=O)C(C(=O)OC(C)(C)C)c1nc(C(=O)OC)c([N+](=O)[O-])cc1C(F)(F)F]

To a solution of methyl 6-chloro-3-nitro-5-(trifluoromethyl)pyridine-2-carboxylate (10 g, 35.140 mmol) and K₂CO₃ (9.8 g, 70.909 mmol) in DMF (35 mL) was added 03-tert-butyl 01-ethyl propanedioate (7.2562 g, 7.3 mL, 38.551 mmol) at room temperature. The solution changed color (pale yellow to thick dark red) within 5 min after the addition of t-butyl ethyl malonate. The reaction was stirred 12 h at room temperature then was quenched with H₂O (100 mL) and saturated aqueous NH₄Cl was added until pH=7 was achieved, then extracted the mixture with ethyl acetate (300 mL), washed with brine (2×90 mL), dried over Na₂SO₄, filtered and concentrated in vacuo. The crude residue was purified by silica gel chromatography using a gradient from 0% to 20% ethyl acetate in hexanes (the hexanes containing 0.1% acetic acid modifier) to provide 01-tert-butyl 03-ethyl 2-[6-methoxycarbonyl-5-nitro-3-(trifluoromethyl)-2-pyridyl]propanedioate (16 g, 99%) as a golden yellow oil. ¹H NMR (500 MHz, Chloroform-d) δ 8.69 (s, 1H), 5.17 (s, 1H), 4.37-4.19 (m, 2H), 4.01 (s, 3H), 1.47 (s, 9H), 1.31-1.24 (m, 3H) ppm. ESI-MS m/z calc. 436.1094, found 437.2 (M+1)⁺; Retention time: 6.09 minutes (LC Method DD).

### Step 2: Methyl 6-(2-ethoxy-2-oxo-ethyl)-3-nitro-5-(trifluoromethyl)pyridine-2-carboxylate

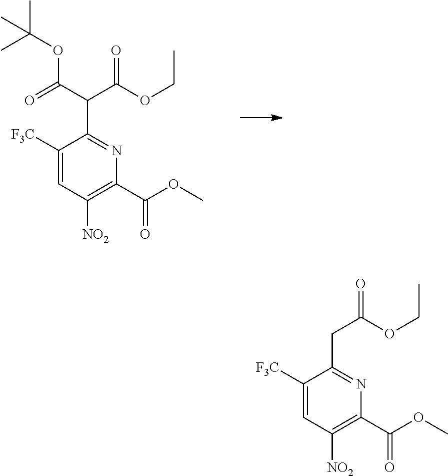 [SMILES: CCOC(=O)C(C(=O)OC(C)(C)C)c1nc(C(=O)OC)c([N+](=O)[O-])cc1C(F)(F)F ; CCOC(=O)Cc1nc(C(=O)OC)c([N+](=O)[O-])cc1C(F)(F)F]

To a solution of 01-tert-butyl 03-ethyl 2-[6-methoxycarbonyl-5-nitro-3-(trifluoromethyl)-2-pyridyl]propanedioate (15.33 g, 35.133 mmol) in DCM (61 mL) was added TFA (75.48 g, 51 mL, 661.97 mmol) slowly at room temperature. The reaction mixture was cooled to 0° C. and saturated aqueous NaHCO₃ (50 mL, until pH=8 to 9) was slowly added then extracted the mixture with DCM (600 mL). The organic phase was dried over Na₂SO₄, filtered and concentrated in vacuo. The amber residue was purified by silica gel chromatography using a gradient from 0% to 25% ethyl acetate in hexanes (hexanes modified with 0.1% acetic acid) and the fractions containing product crystallized and were filtered to provide 4.15 g of pure product. The supernatant was concentrated in vacuo to furnish an additional 3.52 g of methyl 6-(2-ethoxy-2-oxo-ethyl)-3-nitro-5-(trifluoromethyl)pyridine-2-carboxylate (7.67 g, 64%). ¹H NMR (500 MHz, DMSO-d₆) δ 8.95 (s, 1H), 4.24-4.20 (m, 2H), 4.13 (q, J=7.1 Hz, 2H), 3.97 (s, 3H), 1.17 (t, J=7.1 Hz, 3H) ppm. ESI-MS m/z calc. 336.0569, found 337.1 (M+1)⁺; Retention time: 4.92 minutes (LC Method DD).

### Step 3: 6-(2-Ethoxy-2-oxo-ethyl)-3-nitro-5-(trifluoromethyl)pyridine-2-carboxylic Acid

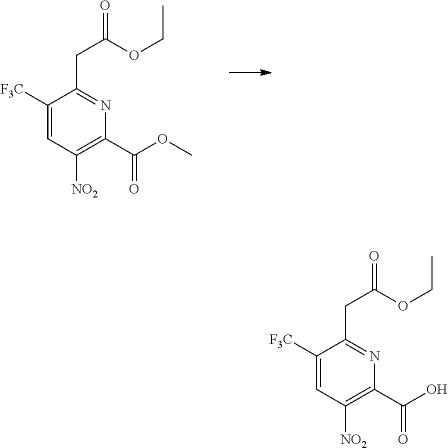 [SMILES: CCOC(=O)Cc1nc(C(=O)OC)c([N+](=O)[O-])cc1C(F)(F)F ; CCOC(=O)Cc1nc(C(=O)O)c([N+](=O)[O-])cc1C(F)(F)F]

To a solution of methyl 6-(2-ethoxy-2-oxo-ethyl)-3-nitro-5-(trifluoromethyl)pyridine-2-carboxylate (1 g, 2.9445 mmol) in THF (3 mL) and water (1 mL) was added lithium hydroxide monohydrate (1.24 g, 29.549 mmol) at room temperature then stirred for 45 min. The reaction was quenched by pouring into saturated aqueous NH₄Cl (100 mL, pH=7 after addition, note: an acidic quench is critical for this reaction as multiple major side products were observed when this was not done) then acidified with HCl (until pH=1) and extracted with ethyl acetate (2×50 mL). The organic solution was then dried over Na₂SO₄, filtered and concentrated in vacuo to provide 6-(2-ethoxy-2-oxo-ethyl)-3-nitro-5-(trifluoromethyl)pyridine-2-carboxylic acid (948 mg, 99%) as a pale yellow solid. ¹H NMR (500 MHz, DMSO-d₆) δ 8.90 (s, 1H), 4.20 (d, J=1.3 Hz, 2H), 4.13 (q, J=7.1 Hz, 2H), 3.41 (s, 1H), 1.17 (t, J=7.1 Hz, 3H) ppm. ESI-MS m/z calc. 322.0413, found 323.3 (M+1)⁺; Retention time: 3.43 minutes (LC Method DD).

### Step 4: Ethyl 2-[6-[[[2-benzyloxy-5-(1,3-dioxolan-2-yl)-2-(trifluoromethyl)pentanoyl]amino]carbamoyl]-5-nitro-3-(trifluoromethyl)-2-pyridyl]acetate

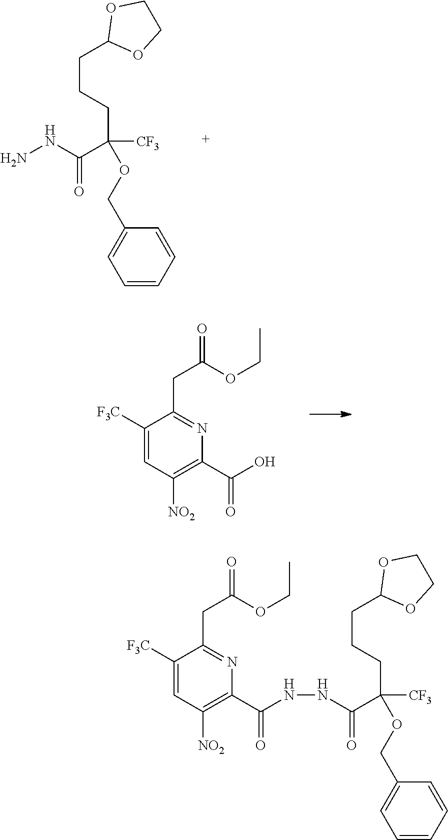 [SMILES: NNC(=O)C(CCCC1OCCO1)(OCc1ccccc1)C(F)(F)F ; CCOC(=O)Cc1nc(C(=O)O)c([N+](=O)[O-])cc1C(F)(F)F ; CCOC(=O)Cc1nc(C(=O)NNC(=O)C(CCCC2OCCO2)(OCc2ccccc2)C(F)(F)F)c([N+](=O)[O-])cc1C(F)(F)F]

To a solution of 6-(2-ethoxy-2-oxo-ethyl)-3-nitro-5-(trifluoromethyl)pyridine-2-carboxylic acid (948 mg, 2.9423 mmol) and 2-benzyloxy-5-(1,3-dioxolan-2-yl)-2-(trifluoromethyl)pentanehydrazide (1.066 g, 2.942 mmol) in ethyl acetate (8 mL) was added T3P in ethyl acetate (2.62 g, 50% w/w, 4.1172 mmol) followed by pyridine (1.0758 g, 1.1 mL, 13.601 mmol) and the mixture was stirred at room temperature. The reaction was quenched with saturated NH₄Cl (30 mL) and extracted with ethyl acetate (3×30 mL). The combined organic layers were washed with brine (100 mL), dried over anhydrous Na₂SO₄, filtered and concentrated in vacuo. The white residue was purified by silica gel chromatography using a gradient from 20% to 50% ethyl acetate in hexanes to provide ethyl 2-[6-[[[2-benzyloxy-5-(1,3-dioxolan-2-yl)-2-(trifluoromethyl)pentanoyl]amino]carbamoyl]-5-nitro-3-(trifluoromethyl)-2-pyridyl]acetate (1.96 g, 98%) as a white solid. ¹H NMR (500 MHz, DMSO-d₆) δ 11.06 (s, 1H), 10.44 (s, 1H), 8.97 (s, 1H), 7.51-7.44 (m, 2H), 7.42-7.35 (m, 2H), 7.35-7.29 (m, 1H), 4.84-4.72 (m, 3H), 4.21 (d, J=1.4 Hz, 2H), 4.14 (q, J=7.1 Hz, 2H), 3.90-3.82 (m, 2H), 3.79-3.71 (m, 2H), 2.30-2.19 (m, 1H), 2.19-2.08 (m, 1H), 1.69-1.59 (m, 2H), 1.56-1.45 (m, 2H), 1.18 (t, J=7.1 Hz, 3H) ppm. ESI-MS m/z calc. 666.176, found 667.4 (M+1)⁺; Retention time: 6.07 minutes (LC Method DD).

### Step 5: Ethyl 2-[6-[5-[1-benzyloxy-4-(1,3-dioxolan-2-yl)-1-(trifluoromethyl)butyl]-1,3,4-oxadiazol-2-yl]-5-nitro-3-(trifluoromethyl)-2-pyridyl]acetate

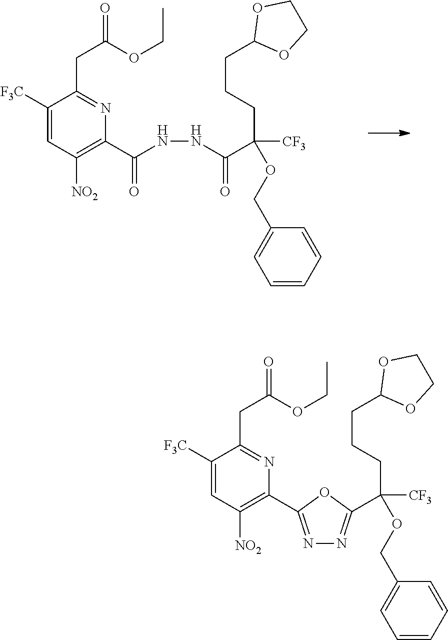 [SMILES: CCOC(=O)Cc1nc(C(=O)NNC(=O)C(CCCC2OCCO2)(OCc2ccccc2)C(F)(F)F)c([N+](=O)[O-])cc1C(F)(F)F ; CCOC(=O)Cc1nc(-c2nnc(C(CCCC3OCCO3)(OCc3ccccc3)C(F)(F)F)o2)c([N+](=O)[O-])cc1C(F)(F)F]

To a solution of ethyl 2-[6-[[[2-benzyloxy-5-(1,3-dioxolan-2-yl)-2-(trifluoromethyl)pentanoyl]amino]carbamoyl]-5-nitro-3-(trifluoromethyl)-2-pyridyl]acetate (5.02 g, 7.5316 mmol) and TsCl (1.7 g, 8.9170 mmol) in CH₃CN (75 mL) was added DIEA (2.968 g, 4 mL, 22.964 mmol) and the mixture was stirred at room temperature for 25 min. The reaction was diluted with ethyl acetate (250 mL) and washed with saturated aqueous NH₄Cl (3×50 mL), brine (50 mL), dried over Na₂SO₄, filtered and concentrated in vacuo. The residue was purified by silica gel chromatography using a gradient from 0% to 25% ethyl acetate in hexanes to provide ethyl 2-[6-[5-[1-benzyloxy-4-(1,3-dioxolan-2-yl)-1-(trifluoromethyl)butyl]-1,3,4-oxadiazol-2-yl]-5-nitro-3-(trifluoromethyl)-2-pyridyl]acetate (3.7 g, 72%) as a golden oil. ¹H NMR (500 MHz, DMSO-d₆) δ 9.13 (s, 1H), 7.43-7.29 (m, 5H), 4.81 (t, J=4.5 Hz, 1H), 4.72 (d, J=10.8 Hz, 1H), 4.60 (d, J=10.8 Hz, 1H), 4.28 (d, J=1.2 Hz, 2H), 4.13 (q, J=7.1 Hz, 2H), 3.90-3.81 (m, 2H), 3.81-3.71 (m, 2H), 2.56-2.51 (m, 1H), 2.48-2.42 (m, 1H), 1.73-1.65 (m, 2H), 1.65-1.51 (m, 2H), 1.17 (t, J=7.1 Hz, 3H) ppm. ESI-MS m/z calc. 648.1655, found 649.6 (M+1)⁺; Retention time: 6.73 minutes (LC Method DD).

### Step 6: Ethyl 2-[5-amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetate

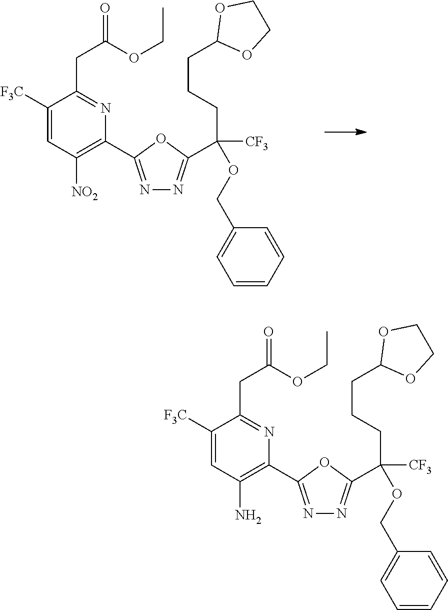

To a solution of ethyl 2-[6-[5-[1-benzyloxy-4-(1,3-dioxolan-2-yl)-1-(trifluoromethyl)butyl]-1,3,4-oxadiazol-2-yl]-5-nitro-3-(trifluoromethyl)-2-pyridyl]acetate (3.5 g, 5.397 mmol) in acetic acid (35 mL) was added iron powder (3.25 g, 58.197 mmol) and the mixture was stirred at 50° C. for 3 h. The reaction was allowed to cool slowly to room temperature over 1 h. The reaction was then quenched by pouring slowly into an oversized beaker containing ice cold saturated aqueous NaHCO₃ (pH=8) then extracted with ethyl acetate (3×500 mL), dried over Na₂SO₄, filtered and concentrated in vacuo to provide ethyl 2-[5-amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetate (3.12 g, 89%) as a sticky tan solid. ESI-MS m/z calc. 618.1913, found 619.4 (M+1)⁺; Retention time: 7.17 minutes (LC Method DD).

### Step 7: 2-[5-Amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetic Acid (Lithium Salt)

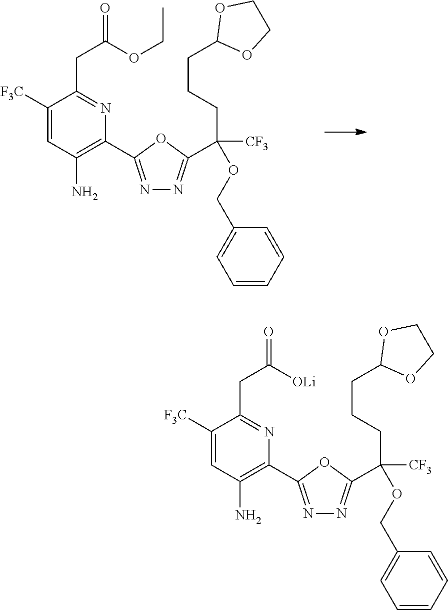

To a solution of crude ethyl 2-[5-amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetate (350 mg, 0.5659 mmol) in THF (3 mL), MeOH (2 mL) and water (2 mL) was added lithium hydroxide monohydrate (240 mg, 5.7192 mmol) and placed the mixture in a 42° C. oil bath with stirring. After 40 minutes, to the reaction was added saturated aqueous NH₄Cl (17 mL, pH=7 to 8). The solvents were removed under reduced pressure and the aqueous was extracted with DCM (3×50 mL). The combined organics were dried over Na₂SO₄, filtered and concentrated in vacuo to provide 2-[5-amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetic acid (lithium salt) (277 mg, 79%) as an off white solid. ¹H NMR (500 MHz, Chloroform-d) δ 7.54 (s, 1H), 7.44-7.27 (m, 5H), 6.12 (d, J=3.8 Hz, 2H), 4.93 (t, J=4.1, 4.1 Hz, 1H), 4.79-4.68 (m, 2H), 4.03-3.99 (m, 2H), 4.00-3.90 (m, 2H), 3.89-3.82 (m, 2H), 2.50-2.35 (m, 2H), 1.89-1.73 (m, 3H), 1.73-1.63 (m, 1H) ppm. ESI-MS m/z calc. 590.16, found 591.1 (M+1)⁺; Retention time: 5.61 minutes (LC Method DD).

### Step 8: 2-[5-Amino-6-[5-[1-benzyloxy-5-oxo-1-(trifluoromethyl)pentyl]-1,3,4-oxadiazol-2-yl]-3-(trifluoromethyl)-2-pyridyl]acetic Acid

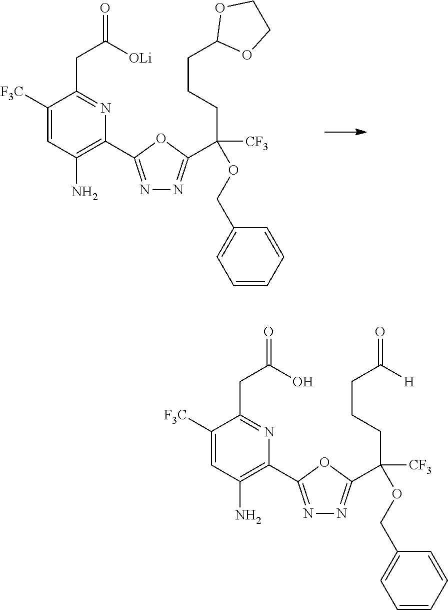

To a suspension of crude 2-[5-amino-6-[5-[1-benzyloxy-4-(1,3-dioxolan-2-yl)-1-(trifluoromethyl)butyl]-1,3,4-oxadiazol-2-yl]-3-(trifluoromethyl)-2-pyridyl]acetic acid (lithium salt) (160 mg, 0.2678 mmol) in HCl (4.3 mL of 3 M, 12.9 mmol) was added acetic acid (8 mL) and stirred the mixture at room temperature for 25 min. The reaction was then diluted with ethyl acetate (50 mL) and poured into cold saturated aqueous NaHCO₃ (˜25 mL, until pH=6), the organics were separated and washed with brine, dried over Na₂SO₄, filtered and concentrated in vacuo at 25° C. (note: the aldehyde appears unstable at higher concentrations and/or in the presence of acid) to provide 2-[5-amino-6-[5-[1-benzyloxy-5-oxo-1-(trifluoromethyl)pentyl]-1,3,4-oxadiazol-2-yl]-3-(trifluoromethyl)-2-pyridyl]acetic acid (148 mg, quant.). ESI-MS m/z calc. 546.1338, found 547.5 (M+1)⁺; Retention time: 5.63 minutes (LC Method DD). The yellow oily residue was used directly in the next step without further purification.

### Step 9: 2-[5-Amino-6-[5-[5-(benzylamino)-1-benzyloxy-1-(trifluoromethyl)pentyl]-1,3,4-oxadiazol-2-yl]-3-(trifluoromethyl)-2-pyridyl]acetic Acid

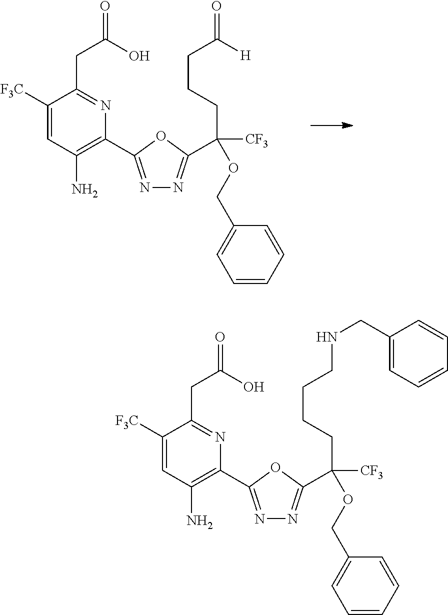 [SMILES: Nc1cc(C(F)(F)F)c(CC(=O)O)nc1-c1nnc(C(CCCC=O)(OCc2ccccc2)C(F)(F)F)o1 ; Nc1cc(C(F)(F)F)c(CC(=O)O)nc1-c1nnc(C(CCCCNCc2ccccc2)(OCc2ccccc2)C(F)(F)F)o1]

To a solution of benzylamine (441.45 mg, 0.45 mL, 4.1198 mmol) in methanol (1.3 mL) was added acetic acid (0.3 mL) and sodium cyanoborohydride (101 mg, 1.6072 mmol) and THE (2 mL) at 0° C. A solution of 2-[5-amino-6-[5-[1-benzyloxy-5-oxo-1-(trifluoromethyl)pentyl]-1,3,4-oxadiazol-2-yl]-3-(trifluoromethyl)-2-pyridyl]acetic acid (220 mg, 0.4026 mmol) in MeOH (5.3 mL) was added dropwise at 0° C. over 5 minutes. The reaction was allowed to stir 30 minutes then quenched slowly with saturated aqueous NaHCO₃ (100 mL) and diluted with MeOH (200 mL) then stirred for 12 h at room temperature and then concentrated in vacuo. The aqueous suspension was diluted with ethyl acetate (120 mL) and water (30 mL) and the layers were separated. The organics were washed with 0.1 M HCl (until pH=1), then a mixture of brine (30 mL) and water (50 mL), dried over Na₂SO₄, filtered and concentrated in vacuo to provide 2-[5-amino-6-[5-[5-(benzylamino)-1-benzyloxy-1-(trifluoromethyl)pentyl]-1,3,4-oxadiazol-2-yl]-3-(trifluoromethyl)-2-pyridyl]acetic acid (246 mg, 67%) as a yellow-orange foam. ¹H NMR (500 MHz, DMSO-d₆) δ 8.80 (s, 1H), 7.79 (s, 1H), 7.57-7.26 (m, 10H), 7.14 (s, 2H), 4.72-4.58 (m, 2H), 4.10 (s, 2H), 3.82 (s, 2H), 2.95 (t, J=7.6 Hz, 2H), 2.43-2.37 (m, 2H), 1.78-1.64 (m, 2H), 1.56 (s, 2H) ppm. ESI-MS m/z calc. 637.2124, found 638.5 (M+1)⁺; Retention time: 5.11 minutes (LC Method DD).

### Step 10: 17-Amino-11-benzyl-6-benzyloxy-6,15-bis(trifluoromethyl)-19-oxa-3,4,11,18-tetrazatricyclo[12.3.1.12,5]nonadeca-1(18),2,4,14,16-pentaen-12-one

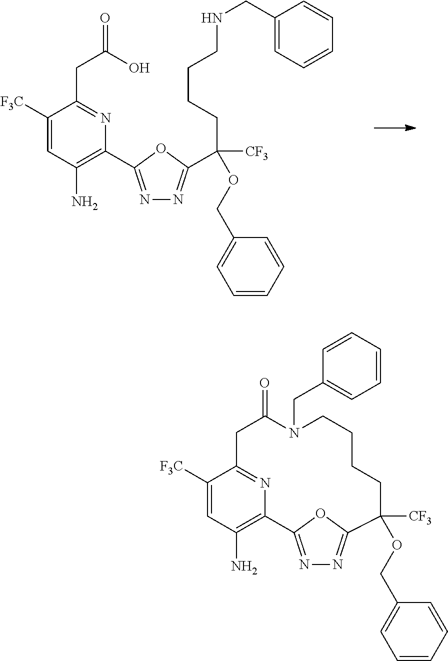 [SMILES: Nc1cc(C(F)(F)F)c(CC(=O)O)nc1-c1nnc(C(CCCCNCc2ccccc2)(OCc2ccccc2)C(F)(F)F)o1 ; Nc1cc(C(F)(F)F)c2nc1C1=NN=C(O1)C(OCc1ccccc1)(C(F)(F)F)CCCCN(Cc1ccccc1)C(=O)C2]

To a solution of DIEA (140.98 mg, 0.19 mL, 1.0908 mmol) in ethyl acetate (1.66 mL) was added T3P in ethyl acetate (175 mg, 50% w/w, 0.275 mmol) at room temperature. A separate flask containing crude 2-[5-amino-6-[5-[5-(benzylamino)-1-benzyloxy-1-(trifluoromethyl)pentyl]-1,3,4-oxadiazol-2-yl]-3-(trifluoromethyl)-2-pyridyl]acetic acid (166 mg, 0.1823 mmol, 70% purity) in ethyl acetate (3.3 mL) and NMP (0.83 mL) was added slowly (25 min dropwise addition) to the flask containing the T3P-DIEA at room temperature. The reaction was stirred for 1.5 h at room temperature then additional T3P (60 mg, 50% w/w, 0.0943 mmol) was added and the resulting mixture was stirred for 30 min. The reaction was diluted with ethyl acetate (50 mL), then washed with water (50 mL), 0.1 M HCl (50 mL, until pH=1), saturated aqueous NaHCO₃ (20 mL, until pH=8) and brine, dried over Na₂SO₄, filtered and concentrated in vacuo. The residue was purified by silica gel chromatography using a gradient from 0% to 40% ethyl acetate in hexanes (hexanes contained 0.5% Et₃N) to provide 17-amino-11-benzyl-6-benzyloxy-6,15-bis(trifluoromethyl)-19-oxa-3,4,11,18-tetrazatricyclo[12.3.1.12,5]nonadeca-1(18),2,4,14,16-pentaen-12-one (52 mg, 66%). ESI-MS m/z calc. 619.2018, found 620.7 (M+1)⁺; Retention time: 6.9 minutes (LC Method DD).

### Step 11: 17-Amino-11-benzyl-6-hydroxy-6,15-bis(trifluoromethyl)-19-oxa-3,4,11,18-tetrazatricyclo[12.3.1.12,5]nonadeca-1(18),2,4,14,16-pentaen-12-one (Compound 51)

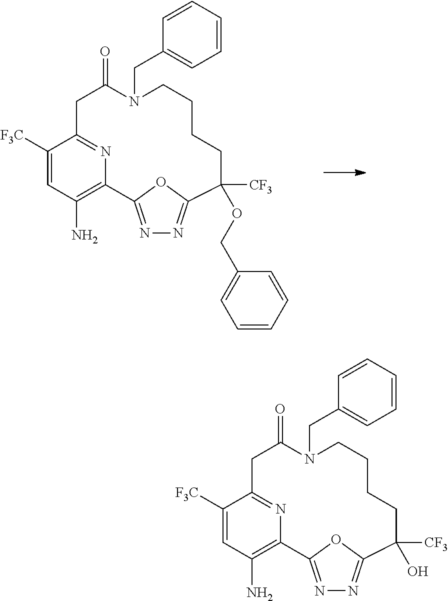 [SMILES: Nc1cc(C(F)(F)F)c2nc1C1=NN=C(O1)C(OCc1ccccc1)(C(F)(F)F)CCCCN(Cc1ccccc1)C(=O)C2 ; Nc1cc(C(F)(F)F)c2nc1C1=NN=C(O1)C(O)(C(F)(F)F)CCCCN(Cc1ccccc1)C(=O)C2]

To a solution of 17-amino-11-benzyl-6-benzyloxy-6,15-bis(trifluoromethyl)-19-oxa-3,4,11,18-tetrazatricyclo[12.3.1.12,5]nonadeca-1(18),2,4,14,16-pentaen-12-one (32 mg, 0.0491 mmol) in EtOH (2 mL) was added 10% Pd/C (16 mg, 0.015 mmol) under nitrogen atmosphere and stirred for 10 minutes at 0° C. The reaction was evacuated and back-filled 3 times with hydrogen gas and stirred at room temperature at 1 atm of hydrogen gas. The reaction was stirred at 30° C. for 24 h then diluted with EtOH (1 mL) and filtered through packed celite, washing with EtOH (3×1 mL). The filtrate was concentrated in vacuo to provide 17-amino-11-benzyl-6-hydroxy-6,15-bis(trifluoromethyl)-19-oxa-3,4,11,18-tetrazatricyclo[12.3.1.12,5]nonadeca-1(18),2,4,14,16-pentaen-12-one (24.8 mg, 92%) as a pale yellow solid. ¹H NMR (500 MHz, DMSO-d₆) δ 7.74 (s, 1H), 7.59 (d, J=37.2 Hz, 1H), 7.40-7.13 (m, 5H), 6.81 (d, J=9.2 Hz, 2H), 4.77-4.10 (m, 3H), 3.96 (dd, J=16.4, 7.6 Hz, 1H), 3.56 (dd, J=16.3, 8.5 Hz, 1H), 2.66-2.56 (m, 1H), 2.30-2.15 (m, 1H), 2.14-1.98 (m, 2H), 1.96-1.80 (m, 2H), 1.73-1.45 (m, 1H) ppm. ESI-MS m/z calc. 529.1549, found 530.2 (M+1)⁺; Retention time: 2.53 minutes (LC Method H).

### Example 30: Preparation of 17-amino-6-hydroxy-11-isopropyl-6,15-bis(trifluoromethyl)-19-oxa-3,4,11,18-tetrazatricyclo[12.3.1.12,5]nonadeca-1(18),2,4,14,16-pentaen-12-one (Compound 52)

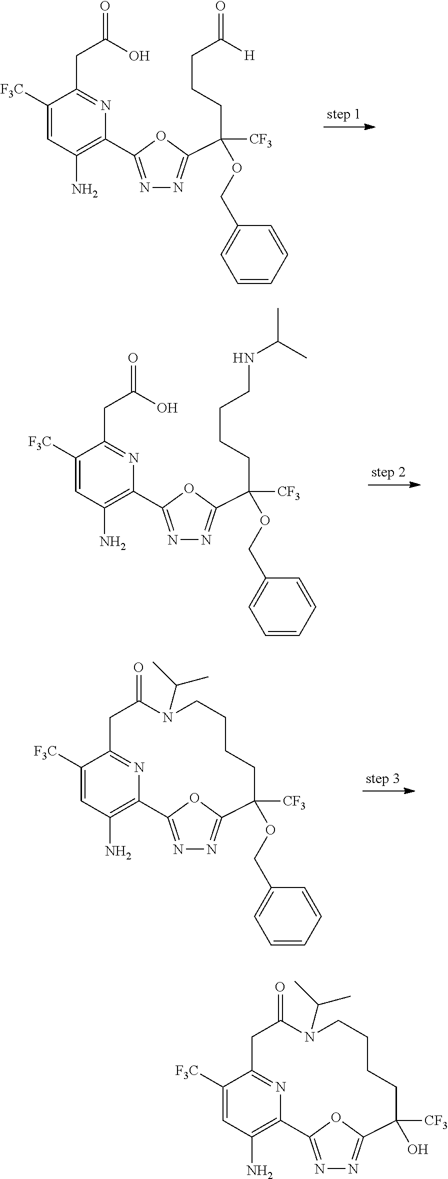

### Step 1: 2-[5-Amino-6-[5-[1-benzyloxy-5-(isopropylamino)-1-(trifluoromethyl)pentyl]-1,3,4-oxadiazol-2-yl]-3-(trifluoromethyl)-2-pyridyl]acetic Acid

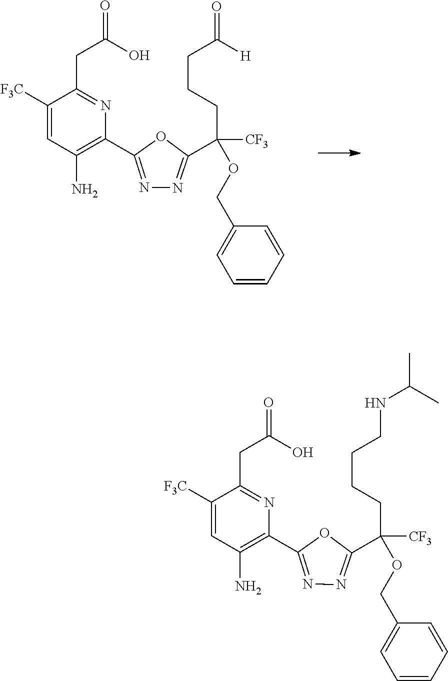 [SMILES: Nc1cc(C(F)(F)F)c(CC(=O)O)nc1-c1nnc(C(CCCC=O)(OCc2ccccc2)C(F)(F)F)o1 ; CC(C)NCCCCC(OCc1ccccc1)(c1nnc(-c2nc(CC(=O)O)c(C(F)(F)F)cc2N)o1)C(F)(F)F]

To a solution of propan-2-amine (242.9 mg, 0.35 mL, 4.1093 mmol) in methanol (1.3 mL) and THE (2 mL) was added acetic acid (0.3 mL) and sodium cyanoborohydride (101 mg, 1.6072 mmol) at 0° C. A solution of 2-[5-amino-6-[5-[1-benzyloxy-5-oxo-1-(trifluoromethyl)pentyl]-1,3,4-oxadiazol-2-yl]-3-(trifluoromethyl)-2-pyridyl]acetic acid (220 mg, 0.4026 mmol) in MeOH (5.3 mL) was added dropwise at 0° C. over 5 min. The reaction was allowed to stir 30 min then quenched slowly with saturated aqueous NaHCO₃ (100 mL) and diluted with MeOH (200 mL) and stirred 12 h at room temperature then concentrated in vacuo. The aqueous suspension was diluted with ethyl acetate (120 mL) and water (30 mL) and the layers were separated. The organics were washed with 0.1 M HCl (until pH=1) followed by a mixture of brine (30 mL) and water (50 mL), dried over Na₂SO₄, filtered and concentrated in vacuo to provide 2-[5-amino-6-[5-[1-benzyloxy-5-(isopropylamino)-1-(trifluoromethyl)pentyl]-1,3,4-oxadiazol-2-yl]-3-(trifluoromethyl)-2-pyridyl]acetic acid (237 mg, 90%) as a pale yellow solid. ESI-MS m/z calc. 589.2124, found 590.2 (M+1)⁺; Retention time: 4.71 minutes. Product was used directly in the next step without further purification (LC Method DD).

### Step 2: 17-Amino-6-benzyloxy-11-isopropyl-6,15-bis(trifluoromethyl)-19-oxa-3,4,11,18-tetrazatricyclo[12.3.1.12,5]nonadeca-1(18),2,4,14,16-pentaen-12-one

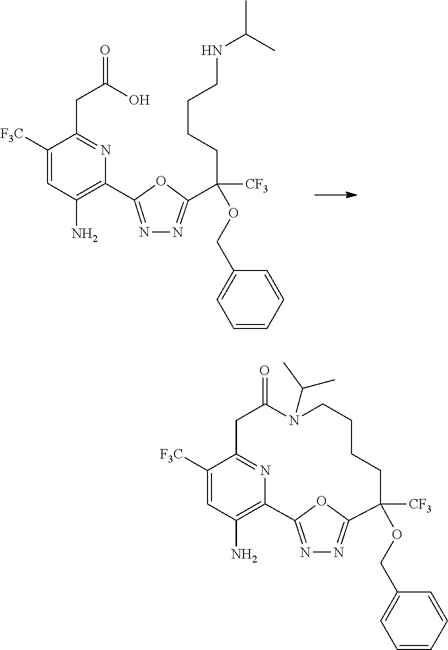

To a solution of DIEA (222.6 mg, 0.3 mL, 1.7223 mmol) in ethyl acetate (2.37 mL) was added T3P in ethyl acetate (359 mg, 50% w/w, 0.5641 mmol) at room temperature. A separate flask containing crude 2-[5-amino-6-[5-[1-benzyloxy-5-(isopropylamino)-1-(trifluoromethyl)pentyl]-1,3,4-oxadiazol-2-yl]-3-(trifluoromethyl)-2-pyridyl]acetic acid (237 mg, 0.2814 mmol, 70% purity) in ethyl acetate (4.74 mL) and NMP (1.185 mL) was added slowly (25 min dropwise addition) to the flask containing T3P-DIEA at room temperature. The reaction was stirred for 2 h at room temperature and then diluted with ethyl acetate (50 mL), washed with water (50 mL), 0.1 M HCl (50 mL, until pH=1), saturated aqueous NaHCO₃ (20 mL, until pH 8) and brine. The organics were then dried over Na₂SO₄, filtered and concentrated in vacuo. The residue was purified by silica gel chromatography using a gradient from 0% to 40% ethyl acetate in hexanes (hexanes contained 0.5% Et₃N) to provide product and mixed product fractions. Mixed product fractions were combined and re-purified by silica gel chromatography using a gradient from 0% to 40% ethyl acetate in hexanes (hexanes contained 0.5% Et₃N) which when combined with product from the first purification gave 17-amino-6-benzyloxy-11-isopropyl-6,15-bis(trifluoromethyl)-19-oxa-3,4,11,18-tetrazatricyclo[12.3.1.12,5]nonadeca-1(18),2,4,14,16-pentaen-12-one (25.9 mg, 23%) as a colorless glass solid. ESI-MS m/z calc. 571.2018, found 572.4 (M+1)⁺; Retention time: 6.53 minutes (LC Method DD).

### Step 3: 17-Amino-6-hydroxy-11-isopropyl-6,15-bis(trifluoromethyl)-19-oxa-3,4,11,18-tetrazatricyclo[12.3.1.12,5]nonadeca-1(18),2,4,14,16-pentaen-12-one (Compound 52)

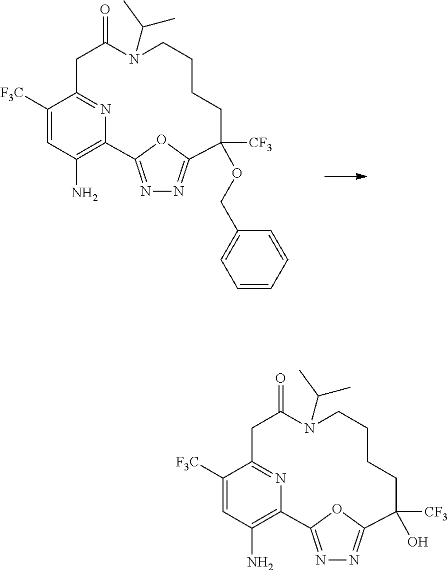 [SMILES: CC(C)N1CCCCC(OCc2ccccc2)(C(F)(F)F)C2=NN=C(O2)c2nc(c(C(F)(F)F)cc2N)CC1=O ; CC(C)N1CCCCC(O)(C(F)(F)F)C2=NN=C(O2)c2nc(c(C(F)(F)F)cc2N)CC1=O]

To a solution of 17-amino-6-benzyloxy-11-isopropyl-6,15-bis(trifluoromethyl)-19-oxa-3,4,11,18-tetrazatricyclo[12.3.1.12,5]nonadeca-1(18),2,4,14,16-pentaen-12-one (25.9 mg, 0.0431 mmol) in EtOH (1.5 mL) was added 10% Pd/C (14 mg, 0.0132 mmol) under nitrogen and stirred the mixture for 10 min at 0° C. The reaction was evacuated and back-filled 3 times with hydrogen gas and stirred at 30° C. at 1 atm of hydrogen gas for 17 h. The reaction was then diluted with EtOH (1 mL) and filtered through packed celite, washing with EtOH (3×1 mL) and the filtrate was concentrated in vacuo to provide 17-amino-6-hydroxy-11-isopropyl-6,15-bis(trifluoromethyl)-19-oxa-3,4,11,18-tetrazatricyclo[12.3.1.12,5]nonadeca-1(18),2,4,14,16-pentaen-12-one (20 mg, 92%) as a pale yellow solid and mixture of isomeric conformers. ESI-MS m/z calc. 481.1549, found 482.5 (M+1)⁺; Retention time: 2.21 minutes (LC Method DD).

### Example 31: Preparation of 17-amino-6-hydroxy-11-methyl-6,15-bis(trifluoromethyl)-19-oxa-3,4,11,18-tetrazatricyclo[12.3.1.12,5]nonadeca-1(18),2,4,14,16-pentaen-12-one (Compound 53)

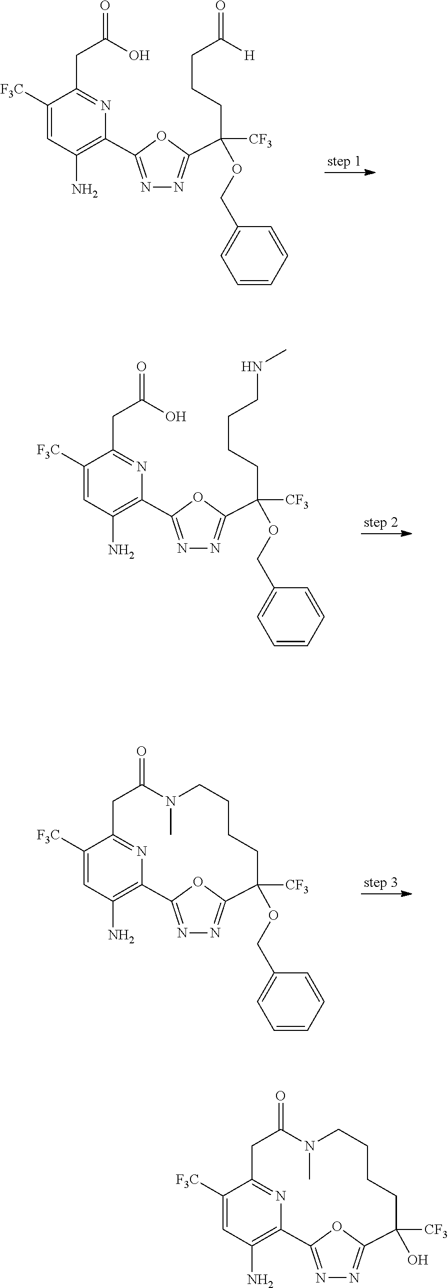 [SMILES: Nc1cc(C(F)(F)F)c(CC(=O)O)nc1-c1nnc(C(CCCC=O)(OCc2ccccc2)C(F)(F)F)o1 ; CNCCCCC(OCc1ccccc1)(c1nnc(-c2nc(CC(=O)O)c(C(F)(F)F)cc2N)o1)C(F)(F)F ; CN1CCCCC(OCc2ccccc2)(C(F)(F)F)C2=NN=C(O2)c2nc(c(C(F)(F)F)cc2N)CC1=O ; CN1CCCCC(O)(C(F)(F)F)C2=NN=C(O2)c2nc(c(C(F)(F)F)cc2N)CC1=O]

### Step 1: 2-[5-Amino-6-[5-[1-benzyloxy-5-(methylamino)-1-(trifluoromethyl)pentyl]-1,3,4-oxadiazol-2-yl]-3-(trifluoromethyl)-2-pyridyl]acetic Acid

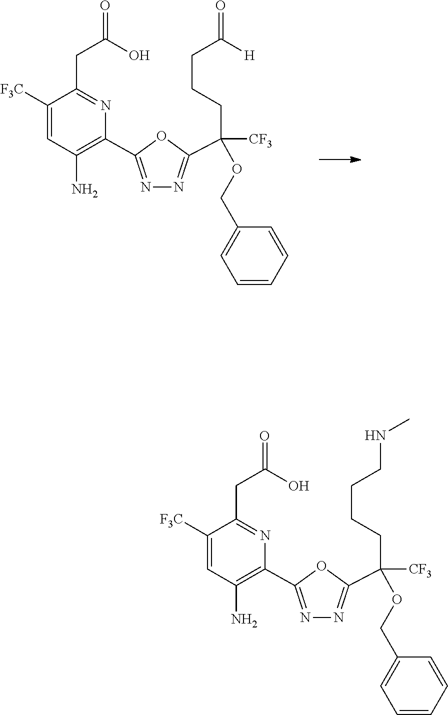 [SMILES: Nc1cc(C(F)(F)F)c(CC(=O)O)nc1-c1nnc(C(CCCC=O)(OCc2ccccc2)C(F)(F)F)o1 ; CNCCCCC(OCc1ccccc1)(c1nnc(-c2nc(CC(=O)O)c(C(F)(F)F)cc2N)o1)C(F)(F)F]

To a solution of methyl amine in THF (2 mL of 2 M, 4 mmol) in methanol (1.3 mL) was added acetic acid (0.3 mL) and sodium cyanoborohydride (101 mg, 1.6072 mmol) at 0° C. A solution of 2-[5-amino-6-[5-[1-benzyloxy-5-oxo-1-(trifluoromethyl)pentyl]-1,3,4-oxadiazol-2-yl]-3-(trifluoromethyl)-2-pyridyl]acetic acid (220 mg, 0.4026 mmol) in methanol (5.3 mL) was added dropwise at 0° C. over 5 minutes. The reaction was allowed to stir 30 minutes then quenched slowly with saturated aqueous NaHCO₃ (100 mL) and diluted with MeOH (200 mL) then stirred 12 h at room temperature and concentrated in vacuo. The aqueous suspension was diluted with ethyl acetate (120 mL) and water (30 mL) and the layers were separated. The organics were washed with 0.1 M HCl (until pH=1) then a mixture of brine (30 mL) and water (50 mL), dried over Na₂SO₄, filtered and concentrated in vacuo to provide 2-[5-amino-6-[5-[1-benzyloxy-5-(methylamino)-1-(trifluoromethyl)pentyl]-1,3,4-oxadiazol-2-yl]-3-(trifluoromethyl)-2-pyridyl]acetic acid (226 mg, 90%) as a yellow solid. ESI-MS m/z calc. 561.1811, found 562.2 (M+1)⁺; Retention time: 4.5 minutes (LC Method DD). The product was used directly in the next step without further purification.

### Step 2: 17-Amino-6-benzyloxy-11-methyl-6,15-bis(trifluoromethyl)-19-oxa-3,4,11,18-tetrazatricyclo[12.3.1.12,5]nonadeca-1(18),2,4,14,16-pentaen-12-one

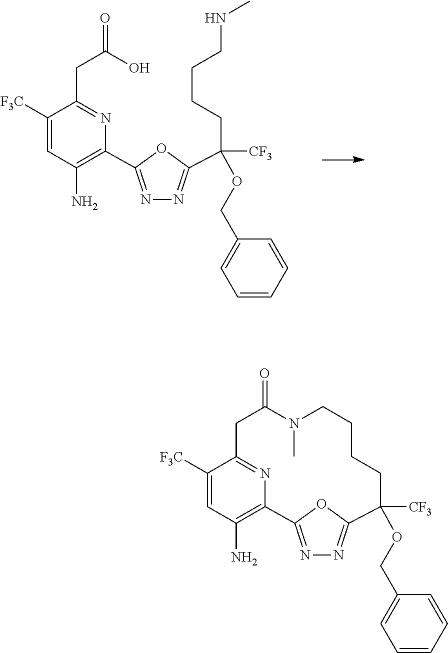 [SMILES: CNCCCCC(OCc1ccccc1)(c1nnc(-c2nc(CC(=O)O)c(C(F)(F)F)cc2N)o1)C(F)(F)F ; CN1CCCCC(OCc2ccccc2)(C(F)(F)F)C2=NN=C(O2)c2nc(c(C(F)(F)F)cc2N)CC1=O]

To a solution of DIEA (222.6 mg, 0.3 mL, 1.7223 mmol) in ethyl acetate (2.26 mL) was added T3P in ethyl acetate (359 mg, 50% w/w, 0.5641 mmol) at room temperature. A separate solution of crude 2-[5-amino-6-[5-[1-benzyloxy-5-(methylamino)-1-(trifluoromethyl)pentyl]-1,3,4-oxadiazol-2-yl]-3-(trifluoromethyl)-2-pyridyl]acetic acid (226 mg, 0.2818 mmol, 70% purity) in ethyl acetate (4.5 mL) and NMP (1.13 mL) was added slowly (25 min dropwise addition) to the flask containing T3P-DIEA at room temperature. The reaction was stirred for 5 h at room temperature then diluted with ethyl acetate (50 mL), washed with water (50 mL), 0.1 M HCl (50 mL, until pH=1), saturated aqueous NaHCO₃ (20 mL, until pH=8) and brine. The organic solution was dried over Na₂SO₄, filtered and concentrated in vacuo. The residue was purified by silica gel chromatography using a gradient from 0% to 40% ethyl acetate in hexanes (hexanes contained 0.5% Et₃N) to provide 17-amino-6-benzyloxy-11-methyl-6,15-bis(trifluoromethyl)-19-oxa-3,4,11,18-tetrazatricyclo[12.3.1.12,5]nonadeca-1(18),2,4,14,16-pentaen-12-one (48.4 mg, 45%). ESI-MS m/z calc. 543.1705, found 544.3 (M+1)⁺; Retention time: 5.87 minutes (LC Method DD).

### Step 3: 17-Amino-6-hydroxy-11-methyl-6,15-bis(trifluoromethyl)-19-oxa-3,4,11,18-tetrazatricyclo[12.3.1.12,5]nonadeca-1(18),2,4,14,16-pentaen-12-one (Compound 53)

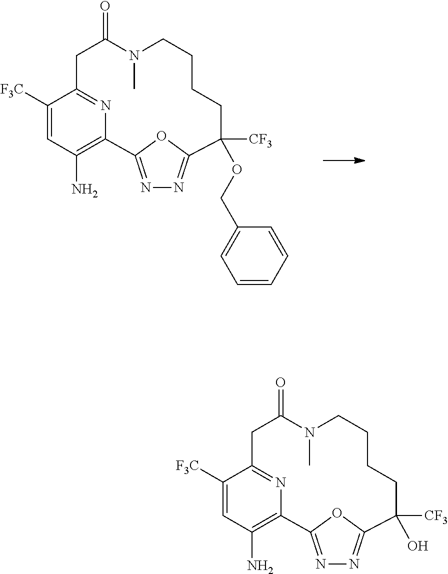 [SMILES: CN1CCCCC(OCc2ccccc2)(C(F)(F)F)C2=NN=C(O2)c2nc(c(C(F)(F)F)cc2N)CC1=O ; CN1CCCCC(O)(C(F)(F)F)C2=NN=C(O2)c2nc(c(C(F)(F)F)cc2N)CC1=O]

To a solution of 17-amino-6-benzyloxy-11-methyl-6,15-bis(trifluoromethyl)-19-oxa-3,4,11,18-tetrazatricyclo[12.3.1.12,5]nonadeca-1(18),2,4,14,16-pentaen-12-one (46 mg, 0.0804 mmol) in EtOH (2.8 mL) was added 10% Pd/C (26 mg, 0.0244 mmol) under nitrogen and stirred the mixture for 10 min at 0° C. The reaction was evacuated and back-filled 3 times with hydrogen gas and stirred at room temperature at 1 atm of hydrogen. After 20 h, the reaction was diluted with EtOH (1 mL) and filtered through packed celite, washing with EtOH (3×1 mL) and the filtrate was concentrated in vacuo to provide 17-amino-6-hydroxy-11-methyl-6,15-bis(trifluoromethyl)-19-oxa-3,4,11,18-tetrazatricyclo[12.3.1.12,5]nonadeca-1(18),2,4,14,16-pentaen-12-one (22.6 mg, 59%) as a pale yellow solid. ¹H NMR (500 MHz, DMSO-d₆) δ 7.74 (s, 1H), 7.55 (d, J=30.9 Hz, 1H), 6.82-6.69 (m, 2H), 4.26-4.12 (m, 2H), 3.50 (dd, J=16.5, 9.5 Hz, 1H), 2.90 (d, J=76.0 Hz, 3H), 2.65-2.54 (m, 1H), 2.26-1.44 (m, 6H) ppm. ESI-MS m/z calc. 453.1236, found 454.3 (M+1)⁺; Retention time: 2.03 minutes (LC Method H).

### Example 32: Preparation of (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (Compound 54), (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (diastereomer 1) (Compound 55), and (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (Diastereomer 2) (Compound 56)

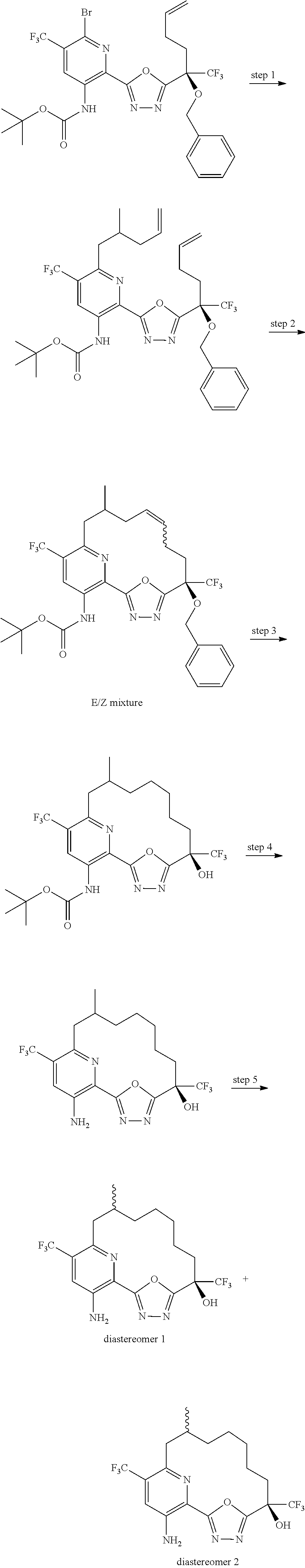

### Step 1: tert-Butyl N-[2-[5-[(1R)-1-benzyloxy-1-(trifluoromethyl)pent-4-enyl]-1,3,4-oxadiazol-2-yl]-6-(2-methylpent-4-enyl)-5-(trifluoromethyl)-3-pyridyl]carbamate

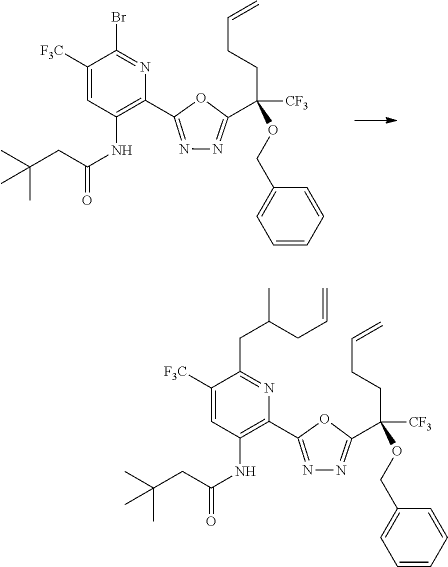

A dried screw-cap test tube, which was equipped with a magnetic stir bar and fitted with a Teflon septum, was charged with tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (500 mg, 0.7676 mmol), palladium acetate (9 mg, 0.0401 mmol), 2-dicyclohexylphosphino-2′,6′-bis(N,N-dimethylamino)biphenyl (34 mg, 0.0779 mmol) and dry THF (0.5 mL). The vessel was evacuated and backfilled with nitrogen (this process was repeated a total of 3 times). The solution was cooled to 0° C. in an ice bath and a solution of bromo(2-methylpent-4-enyl)zinc (4.6 mL of 0.5 M in THF, 2.3 mmol) was added. The ice-bath was removed and the reaction was stirred at room temperature overnight. The reaction mixture was quenched by adding saturated aqueous ammonium chloride solution (5 mL) at 0° C. and extracted with ethyl acetate (80 mL). The organic layer was washed with brine (5 mL), dried over anhydrous sodium sulfate, filtered and concentrated under reduced pressure. The residue was purified by silica gel column chromatography using a gradient from 0% to 10% of ethyl acetate in heptanes to afford tert-butyl N-[2-[5-[(1R)-1-benzyloxy-1-(trifluoromethyl)pent-4-enyl]-1,3,4-oxadiazol-2-yl]-6-(2-methylpent-4-enyl)-5-(trifluoromethyl)-3-pyridyl]carbamate (468 mg, 79%) as a colorless oil. ¹H NMR (400 MHz, DMSO-d₆) δ 9.98 (s, 1H), 8.95 (s, 1H), 7.55-7.23 (m, 5H), 5.96-5.65 (m, 2H), 5.19-4.90 (m, 4H), 4.82-4.60 (m, 2H), 2.93 (dd, J=14.9, 6.4 Hz, 1H), 2.73 (dd, J=14.3, 7.5 Hz, 1H), 2.58-2.52 (m, 1H), 2.44 (br. s., 1H), 2.39-2.21 (m, 3H), 2.20-2.09 (m, 1H), 2.06-1.94 (m, 1H), 1.51 (s, 9H), 0.89 (t, J=6.4 Hz, 3H) ppm. ¹⁹F NMR (377 MHz, DMSO-d₆) δ−59.77 (s, 3F), −72.96 (d, J=15.0 Hz, 3F) ppm. ESI-MS m/z calc. 654.2641, found 655.3 (M+1)⁺; Retention time: 5.38 minutes (LC Method W).

### Step 2: tert-Butyl N-[(6R)-6-benzyloxy-12-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture)

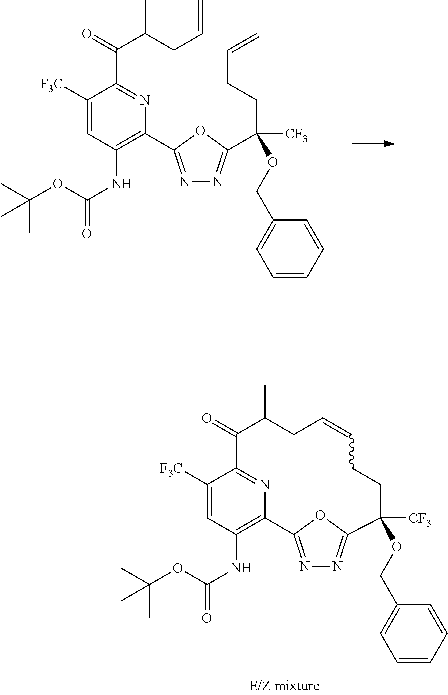 [SMILES: C=CCC[C@@](OCc1ccccc1)(c1nnc(-c2nc(C(=O)C(C)CC=C)c(C(F)(F)F)cc2NC(=O)OC(C)(C)C)o1)C(F)(F)F ; CC1CC=CCC[C@](OCc2ccccc2)(C(F)(F)F)C2=NN=C(O2)c2nc(c(C(F)(F)F)cc2NC(=O)OC(C)(C)C)C1=O]

In a 500 mL three-necked flask equipped with a nitrogen inlet and a reflux condenser, a solution of tert-butyl N-[2-[5-[(1R)-1-benzyloxy-1-(trifluoromethyl)pent-4-enyl]-1,3,4-oxadiazol-2-yl]-6-(2-methylpent-4-enyl)-5-(trifluoromethyl)-3-pyridyl]carbamate (468 mg, 0.6077 mmol) in 1,2-dichloroethane (250 mL) was degassed by bubbling through the solution with nitrogen gas for 18 to 19 hours. A first portion of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (28 mg, 0.0382 mmol) was added after the temperature reached 60° C. After 40 minutes, a second portion of dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (27 mg, 0.0368 mmol) was added and the reaction was left to stir at 60° C. After a total reaction time of 4 hours the flask was cooled to room temperature, the catalyst was quenched with eight drops of DMSO and the reaction was concentrated under reduced pressure. The residue was purified by silica gel column chromatography using a gradient from 0% to 10% ethyl acetate in heptanes to afford 426 mg of tert-butyl N-[(6R)-6-benzyloxy-12-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z mixture) as a yellow oil. This material contained a significant impurity by LCMS and was used directly in the following step without further purification.

### Step 3: tert-Butyl N-[(6R)-6-hydroxy-12-methyl-6,15-bis(trifluoromethyl)-19-oxa-3,4,18-triazatricyclo[12.3.1.12,5]nonadeca-1(18),2,4,14,16-pentaen-17-yl]carbamate

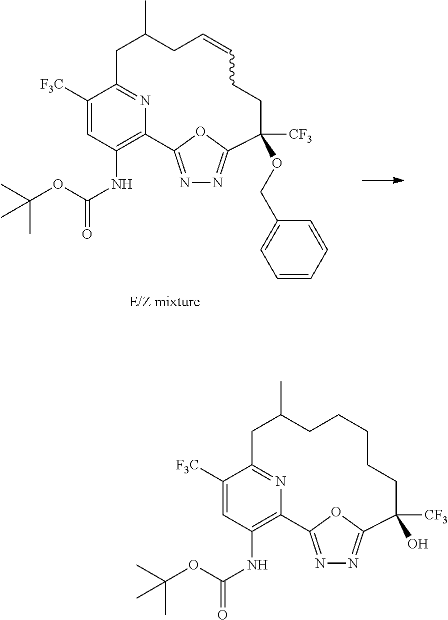 [SMILES: CC1CC=CCC[C@](OCc2ccccc2)(C(F)(F)F)C2=NN=C(O2)c2nc(c(C(F)(F)F)cc2NC(=O)OC(C)(C)C)C1 ; CC1CCCCC[C@](O)(C(F)(F)F)C2=NN=C(O2)c2nc(c(C(F)(F)F)cc2NC(=O)OC(C)(C)C)C1]

A solution of tert-butyl N-[(6R)-6-benzyloxy-12-methyl-6,15-bis(trifluoromethyl)-19-oxa-3,4,18-triazatricyclo[12.3.1.12,5]nonadeca-1(18),2,4,9,14,16-hexaen-17-yl]carbamate (E/Z Mixture) (426 mg) in methanol (12 mL) was purged three times (vacuum then nitrogen atmosphere). Added palladium on carbon (10% w/w, 50% wet, 207 mg, 0.0973 mmol), purged twice under hydrogen atmosphere before leaving the reaction to stir under one atmosphere of hydrogen overnight. Purged once again under nitrogen atmosphere, then the reaction mixture was filtered over a short pad of celite washing with methanol and the filtrate was concentrated. The residue was purified by silica gel chromatography using a gradient from 0% to 10% ethyl acetate in heptanes to afford tert-butyl N-[(6R)-6-hydroxy-12-methyl-6,15-bis(trifluoromethyl)-19-oxa-3,4,18-triazatricyclo[12.3.1.12,5]nonadeca-1(18),2,4,14,16-pentaen-17-yl]carbamate (288 mg, 88% over two steps) as a pale yellow oil. ESI-MS m/z calc. 538.2015, found 539.2 (M+1)⁺; Retention time: 4.65 minutes (LC Method BB).

### Step 4: (6R)-17-Amino-12-methyl-6,15-bis(trifluoromethyl)-19-oxa-3,4,18-triazatricyclo[12.3.1.12,5]nonadeca-1(18),2,4,14,16-pentaen-6-ol (Compound 54)

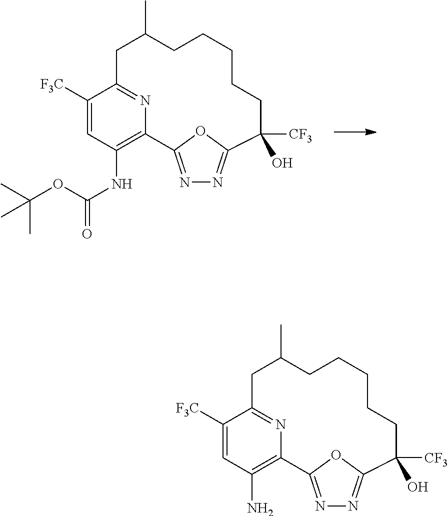 [SMILES: CC1CCCCC[C@](O)(C(F)(F)F)C2=NN=C(O2)c2nc(c(C(F)(F)F)cc2NC(=O)OC(C)(C)C)C1 ; CC1CCCCC[C@](O)(C(F)(F)F)C2=NN=C(O2)c2nc(c(C(F)(F)F)cc2N)C1]

To a solution of tert-butyl N-[(6R)-6-hydroxy-12-methyl-6,15-bis(trifluoromethyl)-19-oxa-3,4,18-triazatricyclo[12.3.1.12,5]nonadeca-1(18),2,4,14,16-pentaen-17-yl]carbamate (201 mg, 0.3733 mmol) in dichloromethane (3 mL) was added trifluoroacetic acid (4.44 g, 3 mL, 38.94 mmol). The mixture was stirred at room temperature for 2 hours. The mixture was then diluted with dichloromethane (10 mL), then concentrated by evaporation under reduced pressure. The residue was dissolved in ethyl acetate (50 mL) then washed with aqueous saturated solution of sodium bicarbonate (3×10 mL) and brine (1×15 mL). The organic phase was then dried with anhydrous sodium sulfate, filtered and concentrated by evaporation under reduced pressure. The residue was purified by silica gel chromatography using a gradient from 0% to 10% of ethyl acetate in dichloromethane. The residue obtained was further purified by a second silica gel chromatography purification using a gradient from 0% to 5% ethyl acetate in dichloromethane. The residue obtained was further purified by reverse-phase C₁₈ prep HPLC using a gradient from 0% to 95% acetonitrile in 0.1% aqueous NH₄HCO₃. The purified product was lyophilized to afford the diastereomeric mixture, (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (62 mg, 38%) as a white solid. ¹H NMR (400 MHz, CDCl₃) δ 7.38 (s, 1H), 5.53 (br. s., 2H), 3.73 (br. s., 1H), 3.05-2.83 (m, 1H), 2.76-2.56 (m, 1H), 2.55-2.16 (m, 3H), 2.15-1.84 (m, 2H), 1.82-1.65 (m, 1H), 1.57-1.39 (m, 3H), 1.13-1.04 (m, 3H), 1.03-0.93 (m, 1H), 0.81-0.65 (m, 1H) ppm. Two diastereomers observed in ¹H NMR. ¹⁹F NMR (377 MHz, CDCl₃) δ−62.25 (s, 3F (minor diastereomer)), −62.64 (s, 3F (major diastereomer)), −78.02 (s, 3F (minor diastereomer)), −80.68 (s, 3F (major diastereomer)) ppm. ESI-MS m/z calc. 438.149, found 439.2 (M+1)⁺; Retention time: 3.59 minutes (LC Method C).

### Step 5: (6R)-17-Amino-12-methyl-6,15-bis(trifluoromethyl)-19-oxa-3,4,18-triazatricyclo[12.3.1.12,5]nonadeca-1(18),2,4,14,16-pentaen-6-ol (diastereomer 1) (Compound 55) and (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (Diastereomer 2) (Compound 56)

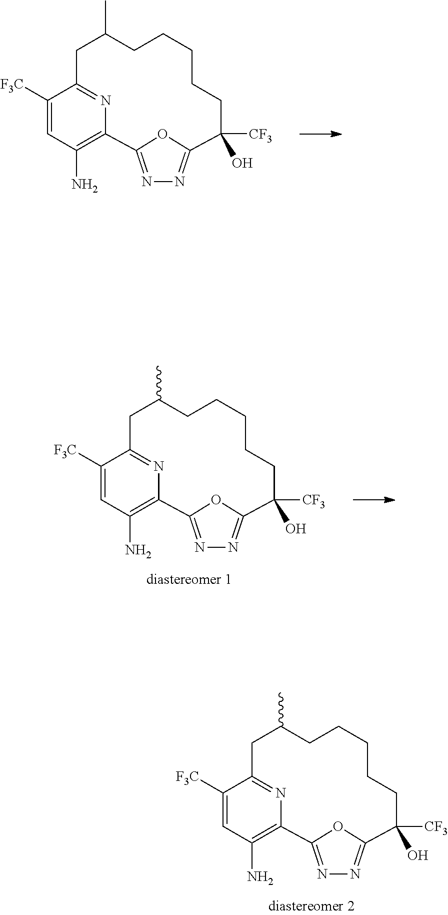

A diastereomeric mixture of (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (45 mg, 0.1027 mmol) was separated into the individual diastereomers by SFC using a Phenomenex LUX-4 column (250×10 mm; 5 μm) at 50° C. (mobile phase was 12% MeOH (+20 mM NH₃), 88% CO₂ at a 10 mL/min flow, concentration of the sample was 20.3 mg/mL in methanol (no modifier), injection volume=70 μL with an outlet pressure of 130 bar, detection wavelength of 224 nm) to afford as the first peak to elute and a white solid, (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (diastereomer 1) (15 mg, 67%). ¹H NMR (400 MHz, DMSO-d₆) δ 7.68 (s, 1H), 7.62 (s, 1H), 6.72 (s, 2H), 2.83 (d, J=17.0 Hz, 1H), 2.58 (dd, J=17.1, 10.3 Hz, 2H), 2.36 (d, J=12.0 Hz, 1H), 2.24 (m, J=14.1, 6.8 Hz, 1H), 2.14-1.97 (m, 2H), 1.60 (d, J=6.0 Hz, 3H), 1.43 (d, J=6.5 Hz, 2H), 1.00 (d, J=6.8 Hz, 3H), 0.82-0.67 (m, 1H) ppm. ESI-MS m/z calc. 438.14905, found 439.4 (M+1)⁺; Retention time: 2.21 minutes (LC Method A).

The second diastereomer to elute was isolated as a white solid, (6R)-17-amino-12-methyl-6,15-bis(trifluoromethyl)-19-oxa-3,4,18-triazatricyclo[12.3.1.12,5]nonadeca-1(18),2,4,14,16-pentaen-6-ol (diastereomer 2) (2.2 mg, 10%). ¹H NMR (400 MHz, Chloroform-d) δ 7.37 (s, 1H), 5.53 (s, 2H), 3.72 (s, 1H), 2.90 (m, J=16.7, 1.8 Hz, 1H), 2.67 (dd, J=16.7, 10.5 Hz, 1H), 2.38-2.18 (m, 3H), 2.04-1.87 (m, 2H), 1.68 (d, J=5.3 Hz, 1H), 1.53-1.45 (m, 3H), 1.26 (p, J=5.4 Hz, 1H), 1.07 (d, J=6.9 Hz, 3H), 1.03-0.94 (m, 1H) ppm. ESI-MS m/z calc. 438.14905, found 439.4 (M+1)⁺; Retention time: 2.18 minutes (LC Method A).

### Example 33: Preparation of 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (enantiomer 1) (Compound 57) and 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (Enantiomer 2) (Compound 58)

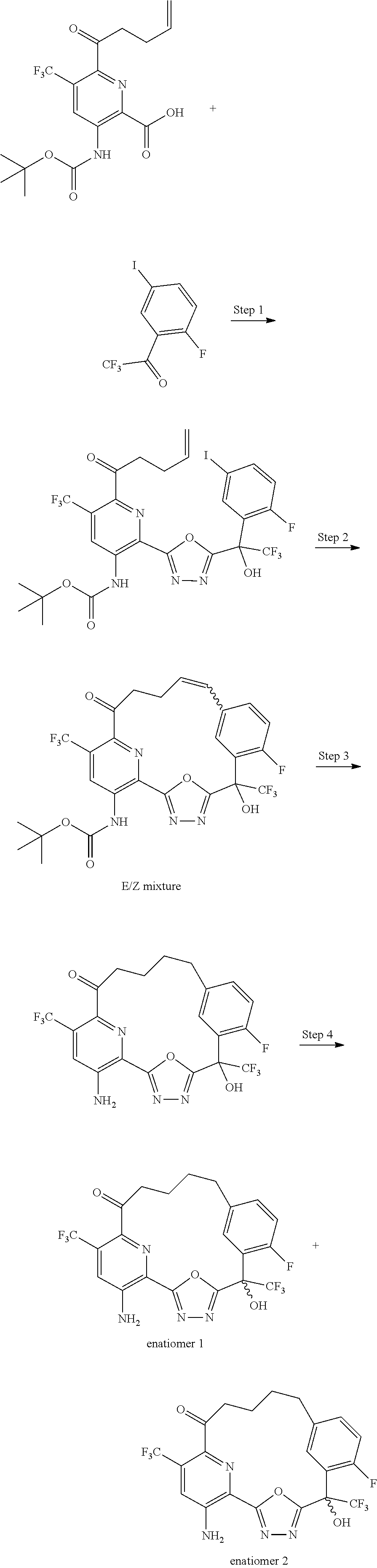

### Step 1: tert-Butyl N-[6-pent-4-enoyl-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate

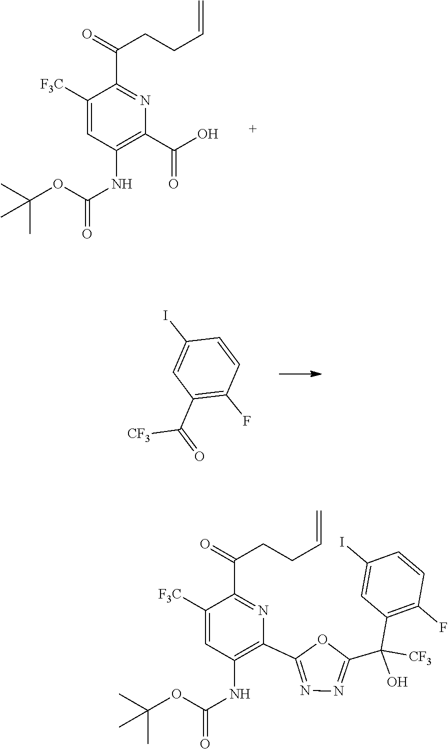 [SMILES: C=CCCC(=O)c1nc(C(=O)O)c(NC(=O)OC(C)(C)C)cc1C(F)(F)F ; O=C(c1cc(I)ccc1F)C(F)(F)F ; C=CCCC(=O)c1nc(-c2nnc(C(O)(c3cc(I)ccc3F)C(F)(F)F)o2)c(NC(=O)OC(C)(C)C)cc1C(F)(F)F]

To a stirred solution of 3-(tert-butoxycarbonylamino)-6-pent-4-enoyl-5-(trifluoromethyl)pyridine-2-carboxylic acid (300 mg, 0.7725 mmol) and 2,2,2-trifluoro-1-(2-fluoro-5-iodo-phenyl)ethanone (256 mg, 0.8050 mmol) in DMF (5 mL) was added (isocyanoimino)triphenylphosphorane (248 mg, 0.8204 mmol) at once. The mixture was stirred at room temperature for 15 min. The material was purified by reverse phase HPLC using a gradient from 50% to 99% acetonitrile in water (+5 mM HCl) over 20 minutes to provide tert-butyl N-[6-pent-4-enoyl-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (146 mg, 37%). ESI-MS m/z calc. 730.0523, found 731.0 (M+1)⁺; Retention time: 0.58 minutes (LC Method T).

### Step 2: tert-Butyl N-[8-fluoro-6-hydroxy-16-oxo-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (E/Z Mixture)

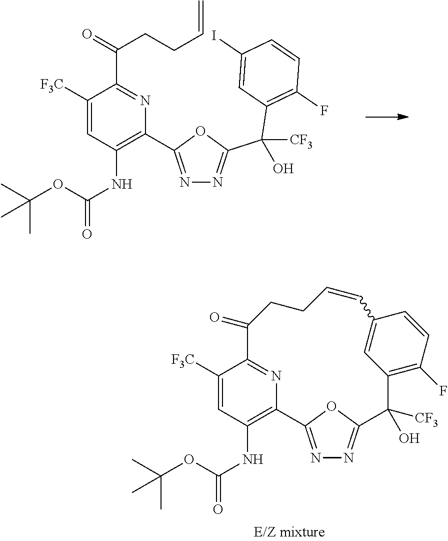 [SMILES: C=CCCC(=O)c1nc(-c2nnc(C(O)(c3cc(I)ccc3F)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(O)(C(F)(F)F)c1cc(ccc1F)C=CCCC2=O]

To a stirred solution of tert-butyl N-[6-pent-4-enoyl-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (427 mg, 0.5846 mmol) in acetonitrile (100 mL) was added palladium (II) acetate (21 mg, 0.09354 mmol) followed by tris-o-tolylphosphane (57 mg, 0.1873 mmol) and triethylamine (600 μL, 4.305 mmol) and the solution was bubbled with N₂ for 1 min then heated at 80° C. for 16 h. Cooled the mixture to room temperature, filtered through Celite and concentrated. The resultant dark brown residue was purified by silica gel column chromatography using a shallow gradient 100% hexanes to 100% ethyl acetate to afford tert-butyl N-[8-fluoro-6-hydroxy-16-oxo-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (E/Z mixture) (170 mg, 48%). ESI-MS m/z calc. 602.14, found 603.3 (M+1)⁺; Retention time: 1.59 minutes and 1.75 minutes (two peaks with same mass, LC Method J)

### Step 3: 20-Amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one

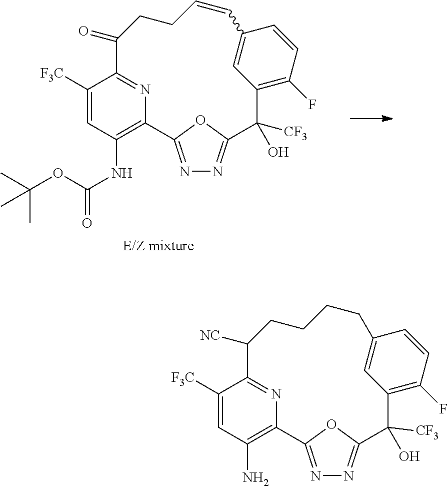 [SMILES: CC(C)(C)OC(=O)Nc1cc(C(F)(F)F)c2nc1C1=NN=C(O1)C(O)(C(F)(F)F)c1cc(ccc1F)C=CCCC2=O ; N#CC1CCCCc2ccc(F)c(c2)C(O)(C(F)(F)F)C2=NN=C(O2)c2nc1c(C(F)(F)F)cc2N]

To a solution of tert-butyl N-[8-fluoro-6-hydroxy-16-oxo-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (E/Z mixture) (52 mg, 0.08631 mmol) in ethanol (5 mL) was added Pd/C (32 mg of 10% w/w, 0.03007 mmol) in a round bottom flask equipped with a H₂ balloon using a 3-way adaptor. Subjected to vacuum and backfilled with nitrogen gas three times then subjected to vacuum. Filled the flask with hydrogen gas then stirred the mixture for 15 hours. The flask was subjected to vacuum and backfilled with nitrogen gas three times then diluted with ethyl acetate and filtered over Celite. Filtrate was concentrated and the residue was dissolved in a pre-made solution of TFA (200 μL, 3.245 mmol) and dichloromethane (600 μL). Stirred the reaction for about 1 h and the solvents were evaporated. The resultant residue was dissolved in 1 mL of DMSO and purified by reverse phase HPLC using a gradient from 30% to 99% acetonitrile in water (+5 mM HCl) over 15 minutes which gave as a white solid, racemic 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (19.7 mg, 45%). ¹H NMR (400 MHz, methanol-d4) δ 8.01 (dd, J=7.7, 2.2 Hz, 1H), 7.67 (s, 1H), 7.30 (m, 1H), 7.12-6.97 (m, 1H), 2.91-2.77 (m, 4H), 1.98-1.85 (m, 2H), 1.75 (p, J=8.2 Hz, 2H) ppm. ESI-MS m/z calc. 504.10324, found 505.0 (M+1)⁺; Retention time: 1.21 minutes (LC Method J).

### Step 4: 20-Amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (enantiomer 1) (Compound 57) and 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (Enantiomer 2) (Compound 58)

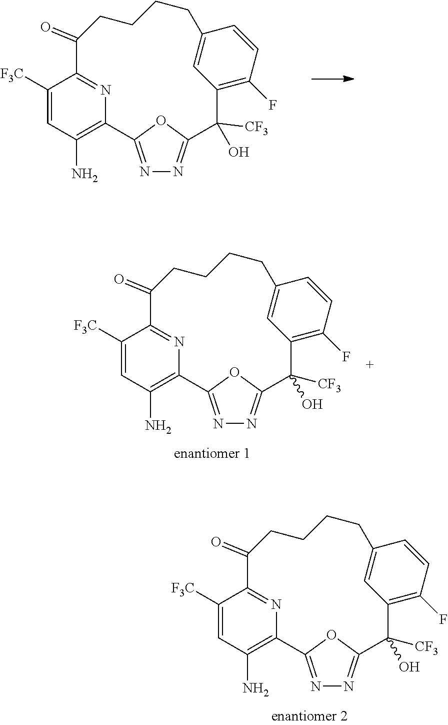 [SMILES: Nc1cc(C(F)(F)F)c2nc1C1=NN=C(O1)C(O)(C(F)(F)F)c1cc(ccc1F)CCCCC2=O ; Nc1cc(C(F)(F)F)c2nc1C1=NN=C(O1)C(O)(C(F)(F)F)c1cc(ccc1F)CCCCC2=O ; Nc1cc(C(F)(F)F)c2nc1C1=NN=C(O1)C(O)(C(F)(F)F)c1cc(ccc1F)CCCCC2=O]

A racemic mixture of 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (18 mg, 0.03569 mmol) was purified by chiral SFC using ChiralPak AD column (250×10 mm, 5 μm particle size) with 10% methanol (20 mM NH₃) and 90% carbon dioxide mobile phase at a flow rate 10 mL/min (injection volume=70 μL of −22 mg/mL solution in methanol (20 mM NH₃)) giving as a white solid and the first enantiomer to elute, 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (enantiomer 1) (4.7 mg, 52%). ¹H NMR (500 MHz, DMSO-d₆) δ 8.90-8.70 (m, 1H), 7.94 (d, J=7.5 Hz, 1H), 7.77 (s, 1H), 7.69-7.43 (m, 2H), 7.33 (t, J=5.7 Hz, 1H), 7.14 (dd, J=12.3, 8.3 Hz, 1H), 2.84 (m, 2H), 2.66 (m, 1H), 2.57 (s, 1H), 1.81 (m, 2H), 1.72 (q, J=7.0, 6.5 Hz, 1H), 1.57 (d, J=13.2 Hz, 1H) ppm. ESI-MS m/z calc. 504.10324, found 505.0 (M+1)⁺; Retention time: 1.21 minutes (LC Method J).

The second eluting enantiomer was isolated as a white solid, 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,17,19-octaen-16-one (enantiomer 2) (4.7 mg, 52%). ¹H NMR (500 MHz, DMSO-d₆) δ 8.78 (s, 1H), 7.93 (d, J=7.4 Hz, 1H), 7.77 (s, 1H), 7.58 (s, 2H), 7.34 (t, J=5.6 Hz, 1H), 7.20-7.08 (m, 1H), 2.84 (m, 2H), 2.72-2.62 (m, 1H), 2.57 (s, 1H), 1.82 (m, 2H), 1.72 (s, 1H), 1.58 (s, 1H) ppm. ESI-MS m/z calc. 504.10324, found 505.0 (M+1)⁺; Retention time: 1.21 minutes (LC Method J).

### Example 34: Preparation of 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (hydrochloride salt) (racemic isomer 1) (Compound 59) and 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (Hydrochloride Salt) (Racemic Isomer 2) (Compound 60)

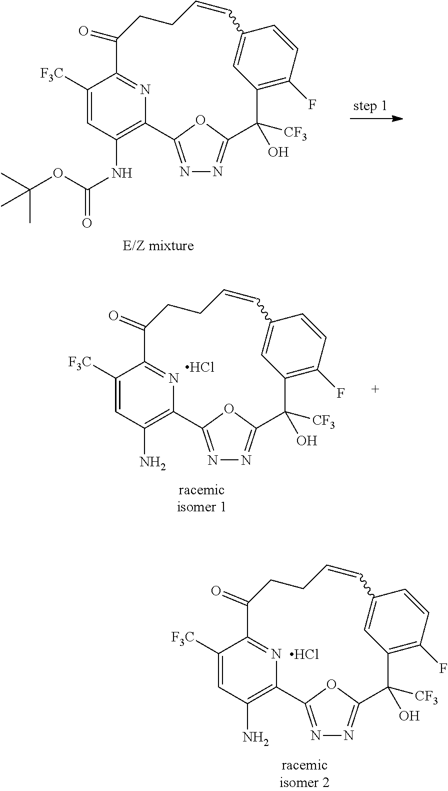 [SMILES: CC(C)(C)OC(=O)Nc1cc(C(F)(F)F)c2nc1C1=NN=C(O1)C(O)(C(F)(F)F)c1cc(ccc1F)C=CCCC2=O ; Nc1cc(C(F)(F)F)c2nc1C1=NN=C(O1)C(O)(C(F)(F)F)c1cc(ccc1F)C=CCCC2=O ; Cl ; Nc1cc(C(F)(F)F)c2nc1C1=NN=C(O1)C(O)(C(F)(F)F)c1cc(ccc1F)C=CCCC2=O ; Cl]

### Step 1: 20-Amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (hydrochloride salt) (racemic isomer 1) (Compound 59) and 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (Hydrochloride Salt) (Racemic Isomer 2) (Compound 60)

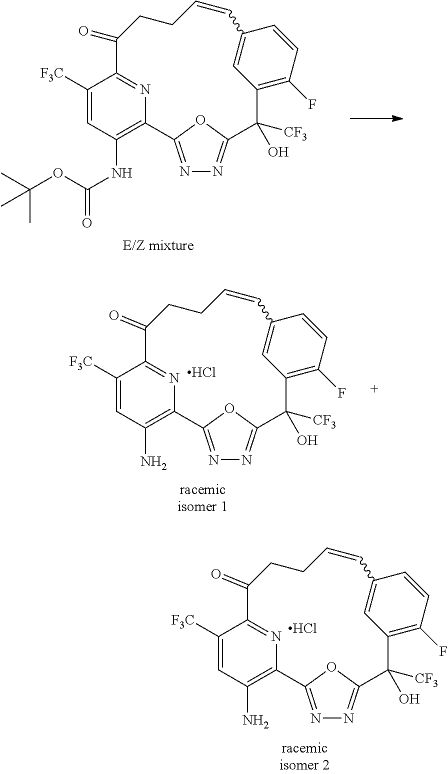 [SMILES: CC(C)(C)OC(=O)Nc1cc(C(F)(F)F)c2nc1C1=NN=C(O1)C(O)(C(F)(F)F)c1cc(ccc1F)C=CCCC2=O ; Nc1cc(C(F)(F)F)c2nc1C1=NN=C(O1)C(O)(C(F)(F)F)c1cc(ccc1F)C=CCCC2=O ; Cl ; Nc1cc(C(F)(F)F)c2nc1C1=NN=C(O1)C(O)(C(F)(F)F)c1cc(ccc1F)C=CCCC2=O ; Cl]

tert-Butyl N-[(8-fluoro-6-hydroxy-16-oxo-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (E/Z mixture) (50 mg, 0.08299 mmol) was dissolved in a pre-made solution of TFA (100 μL, 1.298 mmol) and dichloromethane (400 μL) and the reaction was stirred at room temperature for about 1 h. Solvents were removed and the residue was dissolved in DMSO (1 mL). Purified by reverse phase HPLC using a gradient from 30% to 99% acetonitrile in water (+5 mM HCl) over 30 minutes which gave as a white solid and the first enantiomer to elute, 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (hydrochloride salt) (racemic isomer 1) (11.9 mg, 53%). ¹H NMR (500 MHz, DMSO-d₆) δ 8.56 (s, 1H), 7.85 (s, 1H), 7.56-7.35 (m, 3H), 7.34-7.20 (m, 2H), 7.01-6.89 (m, 1H), 6.16 (d, J=16.1 Hz, 1H), 2.90-2.82 (m, 1H), 2.78-2.73 (m, 1H), 2.59-2.47 (m, 2H) ppm. ESI-MS m/z calc. 502.0876, found 502.9 (M+1)⁺; Retention time: 2.35 minutes (LC Method Q).

The later eluting isomer was isolated as a white solid, 20-amino-8-fluoro-6-hydroxy-6,18-bis(trifluoromethyl)-23-oxa-3,4,21-triazatetracyclo[15.3.1.12,5.17,11]tricosa-1(21),2,4,7(22),8,10,12,17,19-nonaen-16-one (hydrochloride salt) (racemic isomer 2) (8.1 mg, 36%). ¹H NMR (400 MHz, DMSO-d₆) δ 8.76 (d, J=21.7 Hz, 1H), 8.59 (s, 0.5H), 8.08 (s, 0.5H), 7.82 (d, J=4.7 Hz, 1H), 7.38-7.16 (m, 3H), 6.64 (dd, J=7.5, 5.1 Hz, 0.5H), 6.61-6.49 (m, 1H), 5.81 (d, J=11.1 Hz, 0.5H), 2.93-2.82 (m, 2H), 2.79-2.67 (m, 2H) ppm. ESI-MS m/z calc. 502.0876, found 503.0 (M+1)⁺; Retention time: 2.59 minutes (LC Method FF).

### Example 35: Preparation of 20-amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (hydrochloride salt) (diastereomer pair 1) (Compound 61) and 20-amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (hydrochloride salt) (diastereomer pair 2) (Compound 62)

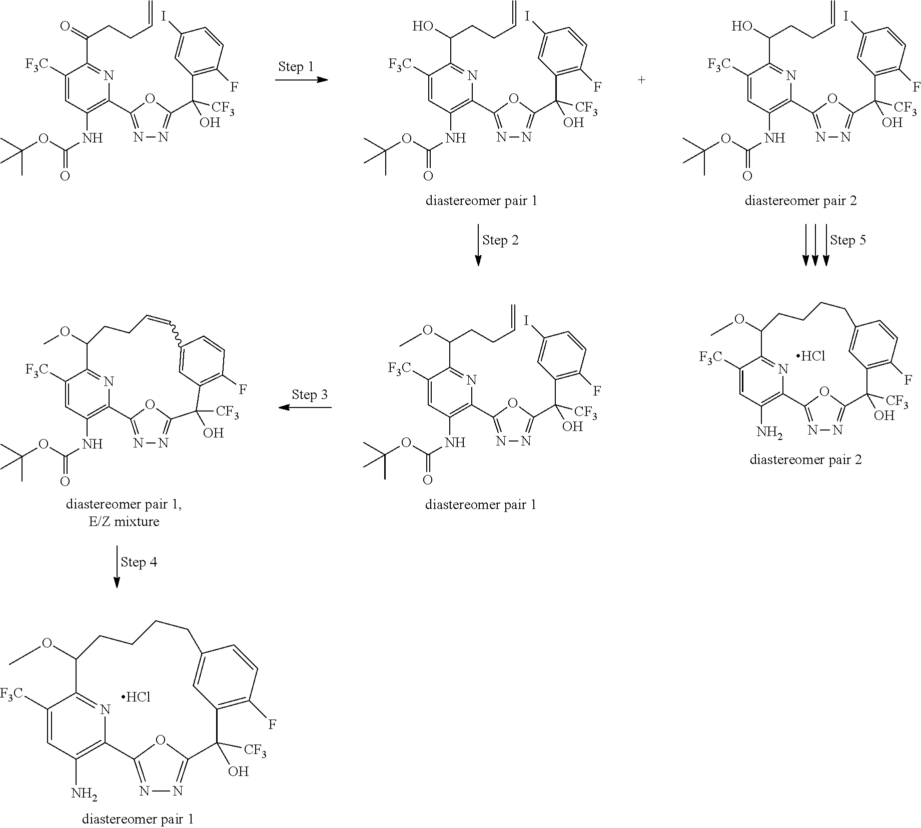

### Step 1: tert-Butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 1) and tert-butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (Diastereomer Pair 2)

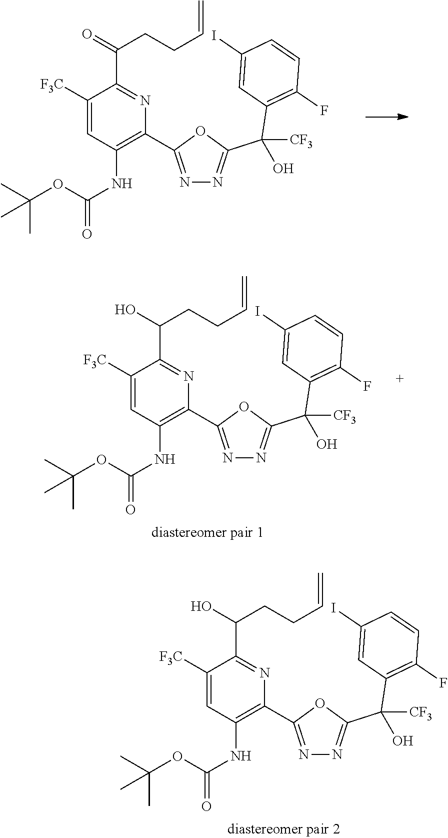 [SMILES: C=CCCC(=O)c1nc(-c2nnc(C(O)(c3cc(I)ccc3F)C(F)(F)F)o2)c(NC(=O)OC(C)(C)C)cc1C(F)(F)F ; C=CCCC(O)c1nc(-c2nnc(C(O)(c3cc(I)ccc3F)C(F)(F)F)o2)c(NC(=O)OC(C)(C)C)cc1C(F)(F)F ; C=CCCC(O)c1nc(-c2nnc(C(O)(c3cc(I)ccc3F)C(F)(F)F)o2)c(NC(=O)OC(C)(C)C)cc1C(F)(F)F]

To a solution of tert-butyl N-[6-pent-4-enoyl-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (140 mg, 0.1917 mmol) in MeOH (2 mL) was added sodium borohydride (11 mg, 0.2908 mmol) at 0° C. and the reaction was stirred for 30 minutes. The reaction was quenched with a few drops of acetic acid and filtered through Whatman filter disc (puradisc 25 TF) and filtrate was purified by reverse phase chromatography using a gradient from 50% to 99% acetonitrile and water (+0.05 mM HCl) over 15 minutes which gave as a colorless solids and first eluting diastereomer pair, tert-butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 1) (58 mg, 83%). ¹H NMR (400 MHz, DMSO-d₆) δ 10.09 (s, 1H), 9.17 (s, 1H), 9.01 (s, 1H), 8.18 (dd, J=7.1, 2.3 Hz, 1H), 7.96 (m, 1H), 7.14 (dd, J=11.4, 8.6 Hz, 1H), 5.79-5.69 (m, 1H), 5.35 (d, J=27.7 Hz, 1H), 5.08-4.89 (m, 2H), 4.82 (s, 1H), 2.12 (m, 1H), 1.97 (m, 1H), 1.89-1.75 (m, 2H), 1.53 (s, 9H) ppm. ESI-MS m/z calc. 732.06793, found 732.9 (M+1)⁺; Retention time: 1.34 minutes (LC Method M).

The second eluting diastereomer pair was isolated as a colorless solid, tert-butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 2) (52 mg, 74%). ¹H NMR (400 MHz, DMSO-d₆) δ 10.05 (s, 1H), 9.20 (s, 1H), 9.00 (s, 1H), 8.19 (dd, J=7.1, 2.3 Hz, 1H), 7.96 (m, 1H), 7.11 (dd, J=11.5, 8.6 Hz, 1H), 5.68 (m, 1H), 5.39 (s, 1H), 4.93-4.84 (m, 2H), 4.80 (d, J=6.4 Hz, 1H), 2.05 (m, 1H), 1.89 (q, J=7.4 Hz, 1H), 1.80 (t, J=6.9 Hz, 2H), 1.53 (s, 9H) ppm. ESI-MS m/z calc. 732.06793, found 733.0 (M+1)⁺; Retention time: 1.37 minutes (LC Method M).

### Step 2: tert-Butyl N-[6-(1-methoxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (Diastereomer Pair 1)

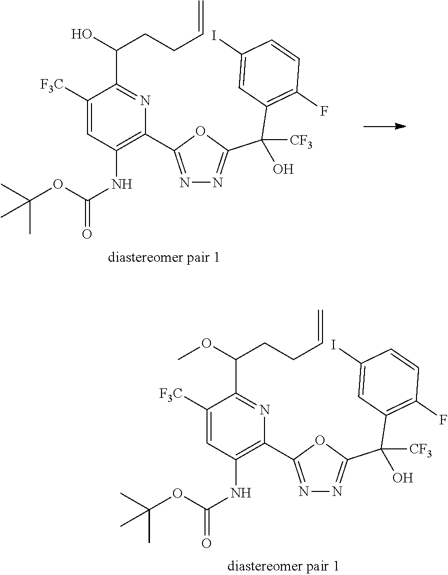 [SMILES: C=CCCC(O)c1nc(-c2nnc(C(O)(c3cc(I)ccc3F)C(F)(F)F)o2)c(NC(=O)OC(C)(C)C)cc1C(F)(F)F ; C=CCCC(OC)c1nc(-c2nnc(C(O)(c3cc(I)ccc3F)C(F)(F)F)o2)c(NC(=O)OC(C)(C)C)cc1C(F)(F)F]

To a stirred solution of tert-butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 1) (55 mg, 0.07510 mmol) in 2-methyltetrahydrofuran (2 mL) at 0° C. (ice bath) was added t-BuOK (50 μL, 0.3832 mmol) (2 M solution in 2-methyltetrahydrofuran) under an inert atmosphere. The mixture was stirred for 5 min and then methyl iodide (10 μL, 0.1606 mmol) was added by a syringe via a rubber septum. The mixture was stirred for additional 30 minutes. The reaction was again cooled in ice bath and more reagents t-BuOK (50 μL, 0.3832 mmol), methyl iodide (10 μL, 0.1606 mmol) were added. After 40 min, the reaction was quenched at 0° C. by the addition of 10 mL water and extracted with diethyl ether (3×20 mL). Organic layers were combined, dried over Na₂SO₄, concentrated and purified silica gel chromatography using a gradient from 100% hexanes to 50% ethyl acetate in hexanes giving, tert-butyl N-[6-(1-methoxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 1) (47 mg, 84%). ESI-MS m/z calc. 746.0836, found 747.3 (M+1)⁺; Retention time: 1.73 minutes (LC Method M).

### Step 3: tert-Butyl N-[8-fluoro-6-hydroxy-16-methoxy-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (Diastereomer Pair 1, E/Z Mixture)

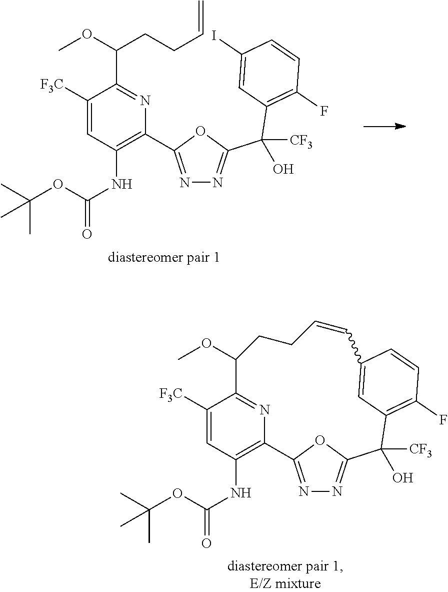 [SMILES: C=CCCC(OC)c1nc(-c2nnc(C(O)(c3cc(I)ccc3F)C(F)(F)F)o2)c(NC(=O)OC(C)(C)C)cc1C(F)(F)F ; COC1CCC=Cc2ccc(F)c(c2)C(O)(C(F)(F)F)C2=NN=C(O2)c2nc1c(C(F)(F)F)cc2NC(=O)OC(C)(C)C]

To a stirred solution of tert-butyl N-[6-(1-methoxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 1) (47 mg, 0.06297 mmol) in acetonitrile (10 mL) was added palladium (II) acetate (3 mg, 0.01336 mmol) followed by tris-o-tolylphosphane (8 mg, 0.02628 mmol) and triethylamine (60 μL, 0.4305 mmol) and the solution was bubbled with N₂ for 2 min then heated at 80° C. for 16 h. Cooled the mixture to room temperature, concentrated down to about 5 mL volume and filtered through Celite and filtrate was concentrated. The resultant brown residue was purified by silica gel chromatography using a shallow gradient from 100% hexanes to 50% ethyl acetate in hexanes to afford tert-butyl N-[8-fluoro-6-hydroxy-16-methoxy-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (diastereomer pair 1, E/Z mixture) 24 mg, 62%). ESI-MS m/z calc. 618.1713, found 619.2 (M+1)⁺; Retention time: 1.41, 1.28, 1.35 and 1.44 minutes (LC Method M). Multiple isomeric products: E/Z isomers at olefin in the macrocycle along with diastereomers; the mixture was taken directly to the next step.

### Step 4: 20-Amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (Hydrochloride Salt) (Diastereomer Pair 1) (Compound 61)

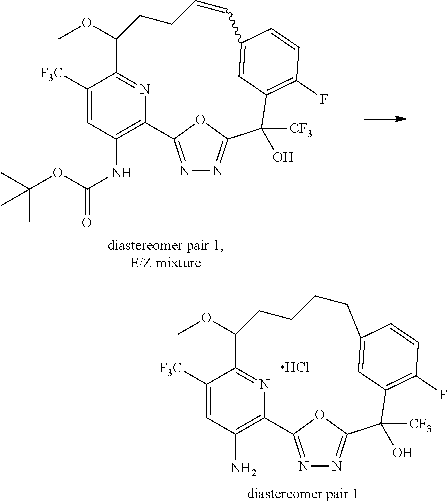 [SMILES: COC1CCC=Cc2ccc(F)c(c2)C(O)(C(F)(F)F)C2=NN=C(O2)c2nc1c(C(F)(F)F)cc2NC(=O)OC(C)(C)C ; COC1CCCCc2ccc(F)c(c2)C(O)(C(F)(F)F)C2=NN=C(O2)c2nc1c(C(F)(F)F)cc2N ; Cl]

To a solution of tert-butyl N-[8-fluoro-6-hydroxy-16-methoxy-6,18-bis(trifluoromethyl)-23-oxa-3,4,21-triazatetracyclo[15.3.1.12,5.17,11]tricosa-1(21),2,4,7(22),8,10,12,17,19-nonaen-20-yl]carbamate (diastereomer pair 1, E/Z mixture) (24 mg, 0.0388 mmol) in ethanol (2 mL) was added Pd/C (6 mg of 10% w/w, 0.005638 mmol) in a round bottom flask equipped with a H₂ balloon using a 3-way adaptor. Subjected to vacuum and backfilled with nitrogen gas three times then subjected to vacuum. Filled the flask with hydrogen gas then stirred the mixture for 15 hours. Subjected to vacuum and backfilled with nitrogen gas three times then diluted with ethyl acetate and filtered over Celite. Filtrate was concentrated and the resultant residue was dissolved in a pre-made solution of TFA (100 μL, 1.298 mmol) and dichloromethane (300 μL) and the reaction was stirred at room temperature for about 1 h. Solvents were removed and dissolved in DMSO (1 mL). The residue was purified by reverse phase chromatography using a gradient from 30% to 99% acetonitrile in water (+5 mM HCl) which gave as a white solid, 20-amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (hydrochloride salt) (diastereomer pair 1) (3.4 mg, 14%). ¹H NMR (400 MHz, DMSO-d₆) δ 8.51 (d, J=91.7 Hz, 1H), 7.71 (d, J=11.3 Hz, 1H), 7.61-7.37 (m, 1H), 7.36-7.29 (m, 1H), 7.22-7.11 (m, 1H), 6.99 (s, 1H), 6.72 (s, 1H), 4.57-4.38 (m, 1H), 3.21 (d, J=6.1 Hz, 3H), 2.87-2.76 (m, 1H), 2.76-2.65 (m, 1H), 1.98 (m, 1H), 1.83 (s, 1H), 1.63 (dd, J=16.8, 10.4 Hz, 2H), 1.48 (d, J=11.5 Hz, 1H), 1.27-1.14 (m, 1H) ppm. ESI-MS m/z calc. 520.1345, found 521.1 (M+1)⁺; Retention time: 1.41 minutes (LC Method J).

### Step 5: 20-Amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (Hydrochloride Salt) (Diastereomer Pair 2) (Compound 62)

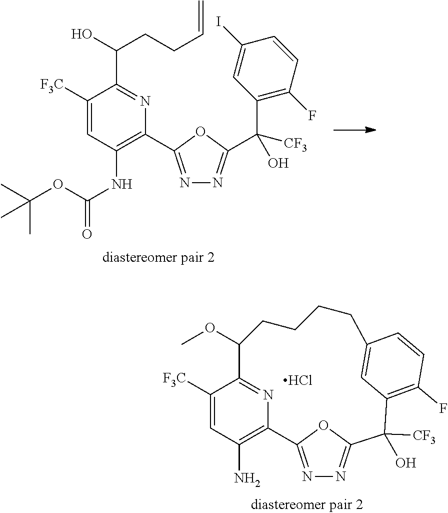 [SMILES: C=CCCC(O)c1nc(-c2nnc(C(O)(c3cc(I)ccc3F)C(F)(F)F)o2)c(NC(=O)OC(C)(C)C)cc1C(F)(F)F ; COC1CCCCc2ccc(F)c(c2)C(O)(C(F)(F)F)C2=NN=C(O2)c2nc1c(C(F)(F)F)cc2N ; Cl]

tert-Butyl N-[6-(1-hydroxypent-4-enyl)-2-[5-[2,2,2-trifluoro-1-(2-fluoro-5-iodo-phenyl)-1-hydroxy-ethyl]-1,3,4-oxadiazol-2-yl]-5-(trifluoromethyl)-3-pyridyl]carbamate (diastereomer pair 2) (55 mg, 0.07510 mmol) was carried through steps 2-4 of this example, analogous to diastereomer pair 1 which provided as a white solid, 20-amino-8-fluoro-16-methoxy-6,18-bis(trifluoromethyl)-23-oxa-3,4,21-triazatetracyclo[15.3.1.12,5.17,11]tricosa-1(21),2,4,7(22),8,10,17,19-octaen-6-ol (hydrochloride salt) (diastereomer pair 2) (3.1 mg, 14%). ¹H NMR (400 MHz, DMSO-d₆) δ 8.66 (s, 1H), 7.69 (s, 1H), 7.37-7.30 (m, 2H), 7.19 (dd, J=12.3, 8.3 Hz, 1H), 7.02 (s, 2H), 4.58-4.47 (m, 1H), 3.06 (s, 3H), 2.74-2.65 (m, 2H), 1.89 (m, 1H), 1.55-1.35 (m, 4H), 1.29-1.18 (m, 1H) ppm. ESI-MS m/z calc. 520.1345, found 521.2 (M+1)⁺; Retention time: 1.45 minutes (LC Method J).

### Example 36: Preparation of 20-amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 1) (Compound 63) and 20-amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (Diastereomer Pair 2) (Compound 64)

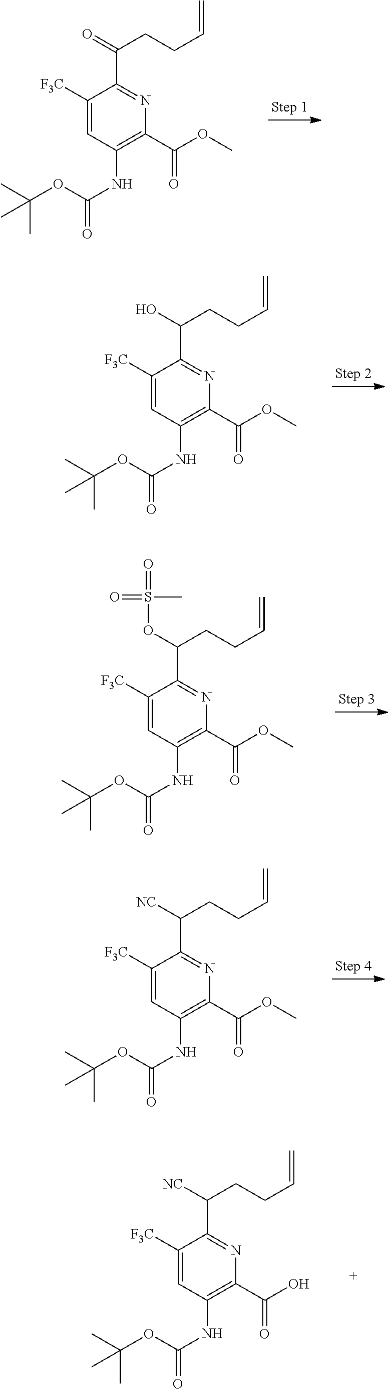

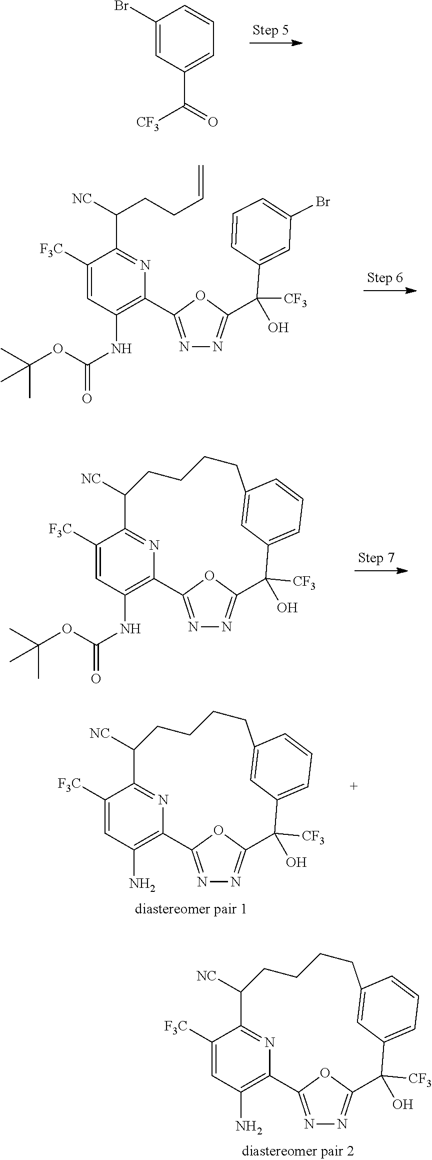

### Step 1: Methyl 3-(tert-butoxycarbonylamino)-6-(1-hydroxypent-4-enyl)-5-(trifluoromethyl)pyridine-2-carboxylate

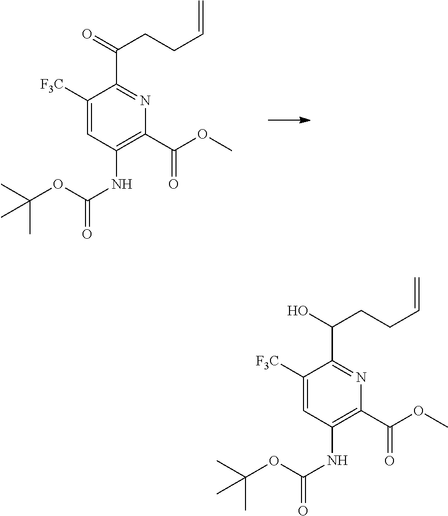 [SMILES: C=CCCC(=O)c1nc(C(=O)OC)c(NC(=O)OC(C)(C)C)cc1C(F)(F)F ; C=CCCC(O)c1nc(C(=O)OC)c(NC(=O)OC(C)(C)C)cc1C(F)(F)F]

To a stirred solution of methyl 3-(tert-butoxycarbonylamino)-6-pent-4-enoyl-5-(trifluoromethyl)pyridine-2-carboxylate (1.1 g, 2.734 mmol) in MeOH (20 mL) in an ice bath was added sodium borohydride (51.7 mg, 1.367 mmol) portion-wise at 0° C. (note: adding NaBH₄ more than 0.5 eq or all at once will reduce the ester to corresponding alcohol). The reaction mixture was stirred for 15 min and quenched by 5% aqueous HCl and extracted with ethyl acetate (3×15 mL). The organic layers were combined, dried over Na₂SO₄, filtered and purified by silica gel chromatography using 80:20 hexanes/ethyl acetate which provided methyl 3-(tert-butoxycarbonylamino)-6-(1-hydroxypent-4-enyl)-5-(trifluoromethyl)pyridine-2-carboxylate (640 mg, 58%). ¹H NMR (400 MHz, CDCl₃) δ 10.14 (s, 1H), 9.22 (s, 1H), 5.83 (m, 1H), 5.05 (dd, J=17.1, 1.8 Hz, 1H), 4.98 (dd, J=10.0, 1.9 Hz, 2H), 4.01 (s, 3H), 3.41 (bs, 1H), 2.38-2.21 (m, 2H), 1.85-1.76 (m, 1H), 1.70-1.58 (m, 1H), 1.54 (s, 9H) ppm. ESI-MS m/z calc. 404.1559, found 405.1 (M+1)⁺; Retention time: 1.46 minutes (LC Method J).

### Step 2: Methyl 3-(tert-butoxycarbonylamino)-6-(1-methylsulfonyloxypent-4-enyl)-5-(trifluoromethyl)pyridine-2-carboxylate

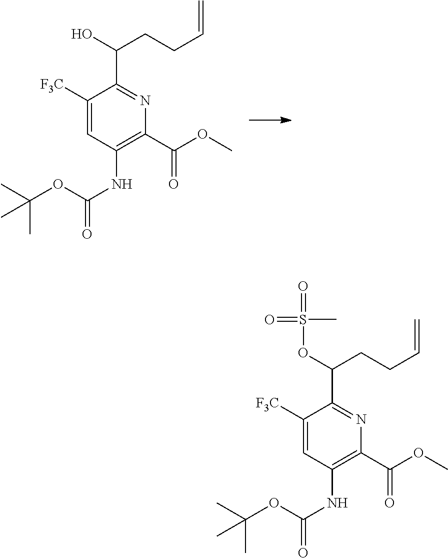 [SMILES: C=CCCC(O)c1nc(C(=O)OC)c(NC(=O)OC(C)(C)C)cc1C(F)(F)F ; C=CCCC(OS(C)(=O)=O)c1nc(C(=O)OC)c(NC(=O)OC(C)(C)C)cc1C(F)(F)F]

To a stirred solution of methyl 3-(tert-butoxycarbonylamino)-6-(1-hydroxypent-4-enyl)-5-(trifluoromethyl)pyridine-2-carboxylate (580 mg, 1.434 mmol) in dichloromethane (15 mL) was added DIEA (556 mg, 4.302 mmol) at room temperature. After 15 min, methane sulfonyl chloride (231 mg, 2.017 mmol) was added dropwise through a rubber septum under inert atmosphere. The reaction mixture was stirred at room temperature and its progress was monitored by UPLC. After 2 hours, the reaction mixture was quenched by saturated solution of NaHCO₃ and extracted with dichloromethane (3×30 mL). The organic phases were combined, dried over Na₂SO₄, concentrated and purified by silica gel chromatography using 75:25 hexanes/ethyl acetate which gave methyl 3-(tert-butoxycarbonylamino)-6-(1-methylsulfonyloxypent-4-enyl)-5-(trifluoromethyl)pyridine-2-carboxylate (385 mg, 56%). ESI-MS m/z calc. 482.13345, found 483.1 (M+1)⁺; Retention time: 1.51 minutes (LC Method J).

### Step 3: Methyl 3-(tert-butoxycarbonylamino)-6-(1-cyanopent-4-enyl)-5-(trifluoromethyl)pyridine-2-carboxylate

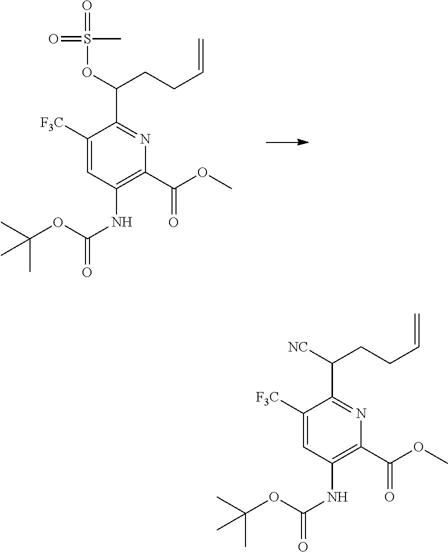 [SMILES: C=CCCC(OS(C)(=O)=O)c1nc(C(=O)OC)c(NC(=O)OC(C)(C)C)cc1C(F)(F)F ; C=CCCC(C#N)c1nc(C(=O)OC)c(NC(=O)OC(C)(C)C)cc1C(F)(F)F]

To a stirred solution of methyl 3-(tert-butoxycarbonylamino)-6-(1-methylsulfonyloxypent-4-enyl)-5-(trifluoromethyl)pyridine-2-carboxylate (385 mg, 0.7980 mmol) in acetonitrile (15 mL) was added tetrabutylammonium cyanide (430 mg, 1.522 mmol). The reaction mixture was heated at 70° C. for 75 minutes then cooled and quenched with a saturated aqueous solution of NaHCO₃ then extracted with ethyl acetate (3×20 mL). The organic phases were combined, dried over Na₂SO₄, filtered and concentrated. The residue was purified by reverse phase chromatography using a gradient from 30% to 95% acetonitrile in water (+5 mM HCl) over 10 minutes which gave methyl 3-(tert-butoxycarbonylamino)-6-(1-cyanopent-4-enyl)-5-(trifluoromethyl)pyridine-2-carboxylate (140 mg, 42%). ESI-MS m/z calc. 413.15625, found 414.1 (M+1)⁺; Retention time: 1.61 minutes (LC Method J).

### Step 4: 3-(tert-Butoxycarbonylamino)-6-(1-cyanopent-4-enyl)-5-(trifluoromethyl)pyridine-2-carboxylic Acid

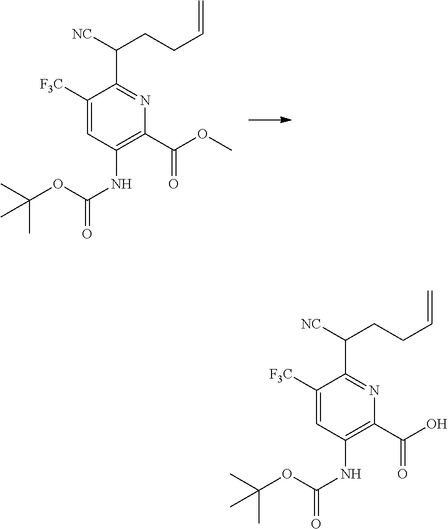 [SMILES: C=CCCC(C#N)c1nc(C(=O)OC)c(NC(=O)OC(C)(C)C)cc1C(F)(F)F ; C=CCCC(C#N)c1nc(C(=O)O)c(NC(=O)OC(C)(C)C)cc1C(F)(F)F]

Into a solution of methyl 3-(tert-butoxycarbonylamino)-6-(1-cyanopent-4-enyl)-5-(trifluoromethyl)pyridine-2-carboxylate (2.648 g, 6.4056 mmol) in THF (24 mL) and water (8 mL) was added LiOH (777 mg, 32.445 mmol) at ambient temperature. The reaction mixture was stirred for 1 hour. The reaction was diluted with saturated ammonium chloride (50 mL) and the pH was adjusted to 5 with 1N HCl. The aqueous solution was extracted with ethyl acetate (3×50 mL). The combined organic layers were washed with brine (50 mL), dried over anhydrous sodium sulfate and concentrated under vacuum to provide 3-(tert-butoxycarbonylamino)-6-(1-cyanopent-4-enyl)-5-(trifluoromethyl)pyridine-2-carboxylic acid (2.572 g, quant.) as a clear gel. ESI-MS m/z calc. 399.1406, found 400.2 (M+1)⁺; Retention time: 3.56 minutes (LC Method G).

### Step 5: tert-Butyl N-[2-[5-[1-(3-bromophenyl)-2,2,2-trifluoro-1-hydroxy-ethyl]-1,3,4-oxadiazol-2-yl]-6-(1-cyanopent-4-enyl)-5-(trifluoromethyl)-3-pyridyl]carbamate

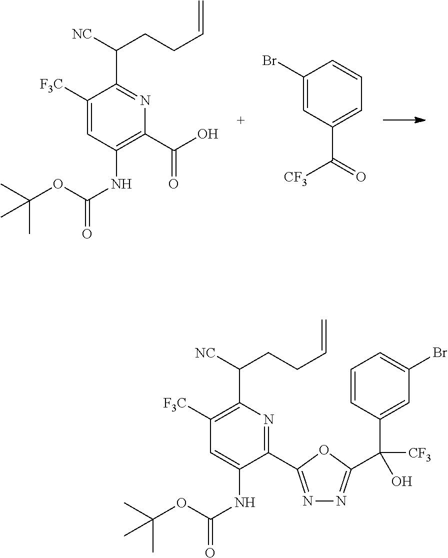 [SMILES: C=CCCC(C#N)c1nc(C(=O)O)c(NC(=O)OC(C)(C)C)cc1C(F)(F)F ; O=C(c1cccc(Br)c1)C(F)(F)F ; C=CCCC(C#N)c1nc(-c2nnc(C(O)(c3cccc(Br)c3)C(F)(F)F)o2)c(NC(=O)OC(C)(C)C)cc1C(F)(F)F]

3-(tert-Butoxycarbonylamino)-6-(1-cyanopent-4-enyl)-5-(trifluoromethyl)pyridine-2-carboxylic acid (180 mg, 0.4507 mmol) and 1-(3-bromophenyl)-2,2,2-trifluoro-ethanone (285 mg, 1.126 mmol) were dissolved in DMF (9 mL) and heated to 45° C. Then, (isocyanoimino)triphenylphosphorane (341 mg, 1.128 mmol) was added and the mixture was stirred at room temperature for 1 hour. The reaction mixture was diluted with ethyl acetate (5 mL), added water (10 mL), and extracted with ethyl acetate (3×10 mL), washed with brine, dried over Na₂SO₄, filtered, and concentered under reduced pressure. The residue was purified by reverse phase chromatography using a gradient from 30% to 100% acetonitrile in water (+5 mM HCl) over 12 minutes which gave as a mixture of stereoisomers, tert-butyl N-[2-[5-[1-(3-bromophenyl)-2,2,2-trifluoro-1-hydroxy-ethyl]-1,3,4-oxadiazol-2-yl]-6-(1-cyanopent-4-enyl)-5-(trifluoromethyl)-3-pyridyl]carbamate (159 mg, 52%). ESI-MS m/z calc. 675.0916, found 678.1 (M+1)⁺; Retention time: 1.55 minutes (LC Method M).

### Step 6: tert-Butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate

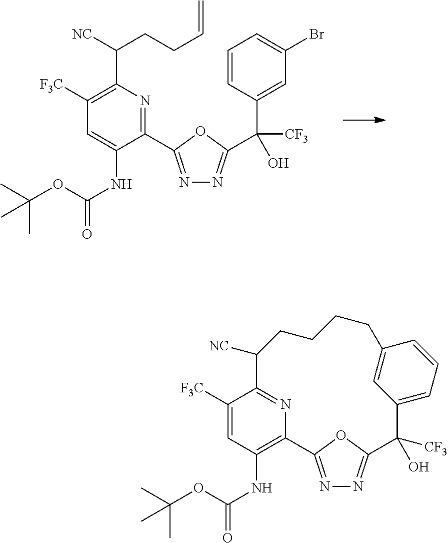 [SMILES: C=CCCC(C#N)c1nc(-c2nnc(C(O)(c3cccc(Br)c3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(O)(C(F)(F)F)c1cccc(c1)CCCCC2C#N]

To a stirred solution of tert-butyl N-[2-[5-[1-(3-bromophenyl)-2,2,2-trifluoro-1-hydroxy-ethyl]-1,3,4-oxadiazol-2-yl]-6-(1-cyanopent-4-enyl)-5-(trifluoromethyl)-3-pyridyl]carbamate (121 mg, 0.1789 mmol) in acetonitrile (7 mL) was added palladium acetate (12.1 mg, 0.05390 mmol) followed by tris-o-tolylphosphane (33.7 mg, 0.1107 mmol) and triethyl amine (56 mg, 0.5534 mmol) and the solution was bubbled with N₂ for 1 min then sealed and heated in a microwave for 2 hours at 125° C. The mixture was cooled to room temperature and filtered through a pad of Celite washing with ethyl acetate. The filtrate was evaporated and the residue was purified by silica gel chromatography using 75:25 hexanes/ethyl acetate giving the desired product as a mixture of diastereomers as well as E/Z isomers. Without further purification, the residue was dissolved in MeOH (3 mL) and transferred to a high-pressure reactor then added 10% Pd/C (19 mg, 0.01785 mmol). The reactor was sealed, filled with nitrogen gas and evacuated three times. Finally, the reactor was evacuated and then filled with H₂ up to 60 psi. The mixture was stirred at room temperature for 6 h. The reactor was depressurized, and the mixture was filtered through a pad of Celite washing with methanol. The filtrate was evaporated and the residue was then purified by reverse phase chromatography using a gradient from 35% to 90% acetonitrile in water (+5 mM HCl) over 10 minutes which gave as mixture of diastereomers, tert-butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (31 mg, 29%). ESI-MS m/z calc. 597.1811, found 598.1 (M+1)⁺; Retention time: 1.79 minutes and 1.84 minutes (LC Method J).

### Step 7: 20-Amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 1) (Compound 63) and 20-amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (Diastereomer Pair 2) (Compound 64)

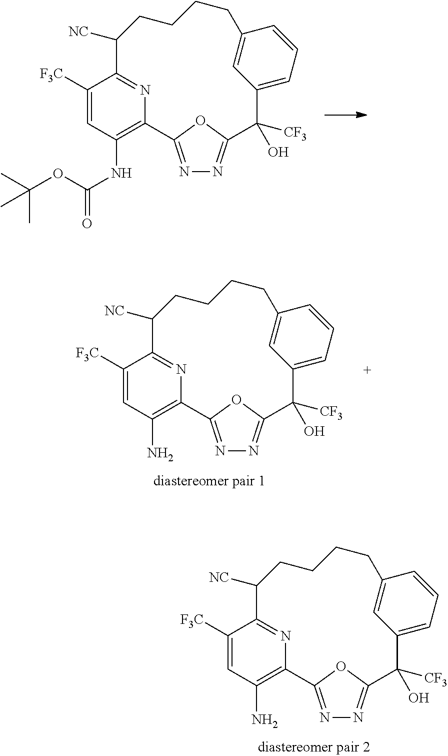 [SMILES: CC(C)(C)OC(=O)Nc1cc(C(F)(F)F)c2nc1C1=NN=C(O1)C(O)(C(F)(F)F)c1cccc(c1)CCCCC2C#N ; N#CC1CCCCc2cccc(c2)C(O)(C(F)(F)F)C2=NN=C(O2)c2nc1c(C(F)(F)F)cc2N ; N#CC1CCCCc2cccc(c2)C(O)(C(F)(F)F)C2=NN=C(O2)c2nc1c(C(F)(F)F)cc2N]

To a stirred solution of tert-butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (14 mg, 0.02343 mmol) in dichloromethane (1 mL) was added TFA (1 mL, 12.98 mmol) and the mixture was stirred for 40 minutes. The solvent was evaporated and the residue was purified by reverse phase chromatography using a gradient from 30% to 100% acetonitrile in water (+5 mM HCl) which gave as the first eluting diastereomer pair, 20-amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 1) (5 mg, 41%). ¹H NMR (400 MHz, CDCl₃) δ 7.95 (d, J=1.9 Hz, 1H), 7.76 (m, 1H), 7.45-7.33 (m, 2H), 7.32-7.19 (m, 1H), 5.98 (bs, 2H), 4.63 (bs, 1H), 4.31 (dd, J=7.1, 2.4 Hz, 1H), 2.94 (m, 1H), 2.74 (m, 1H), 2.48-2.33 (m, 1H), 2.33-2.25 (m, 1H), 1.98-1.80 (m, 2H), 1.81-1.68 (m, 1H), 1.53-1.40 (m, 1H) ppm. ESI-MS m/z calc. 497.12863, found 498.1 (M+1)⁺; Retention time: 1.27 minutes (LC Method J).

The later eluting diastereomer pair provided 20-amino-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2) (2.5 mg, 21%). ¹H NMR (400 MHz, CDCl₃) δ 7.86 (s, 1H), 7.74 (d, J=7.8 Hz, 1H), 7.45-7.34 (m, 2H), 7.28 (d, J=17.1 Hz, 1H), 5.99 (bs, 2H), 4.56 (bs, 1H), 4.38 (dd, J=5.8, 2.7 Hz, 1H), 3.06 (m, 1H), 2.80 (m, 1H), 2.66-2.46 (m, 1H), 2.21-2.04 (m, 2H), 2.02-1.97 (m, 1H), 1.81 (tdd, J=17.4, 8.5, 4.3 Hz, 1H), 1.70-1.61 (m, 1H) ppm. ESI-MS m/z calc. 497.12863, found 498.1 (M+1)⁺; Retention time: 1.31 minutes (LC Method J).

### Example 37: Preparation of 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (enantiomer 1) (Compound 65) and 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (enantiomer 2) (Compound 66)

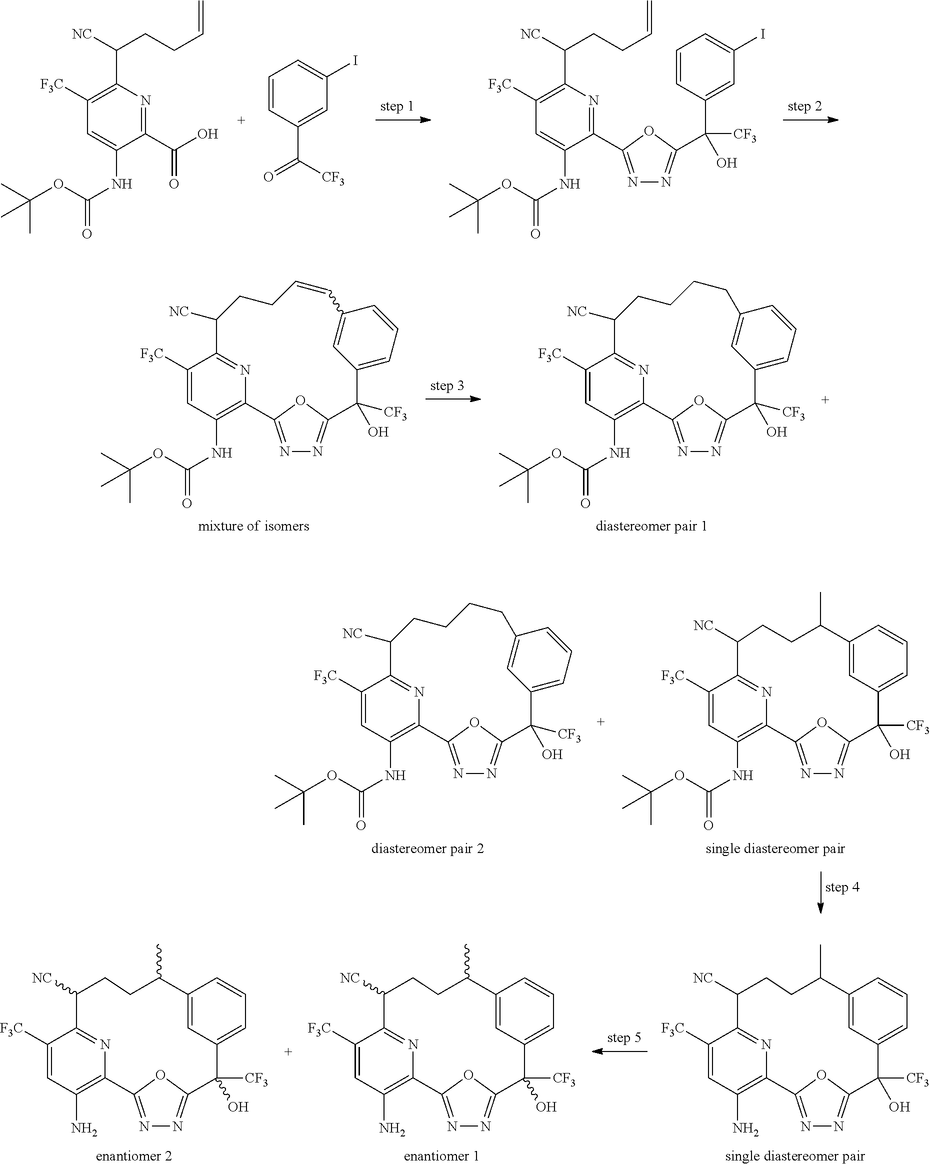

### Step 1: tert-Butyl N-[6-(1-cyanopent-4-enyl)-2-[5-[2,2,2-trifluoro-1-hydroxy-1-(3-iodophenyl)ethyl]-1,3,4-oxadiazol-2-yl]-5-(trifluoromethyl)-3-pyridyl]carbamate

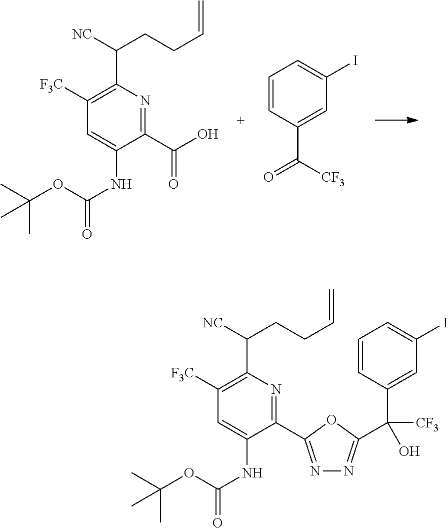 [SMILES: C=CCCC(C#N)c1nc(C(=O)O)c(NC(=O)OC(C)(C)C)cc1C(F)(F)F ; O=C(c1cccc(I)c1)C(F)(F)F ; C=CCCC(C#N)c1nc(-c2nnc(C(O)(c3cccc(I)c3)C(F)(F)F)o2)c(NC(=O)OC(C)(C)C)cc1C(F)(F)F]

A vial was charged with 2,2,2-trifluoro-1-(3-iodophenyl)ethanone (1.078 g, 3.5931 mmol) in DCM (10 mL). (N-isocyanoimino)triphenylphosphorane (1.146 g, 3.6013 mmol) was added to the reaction mixture. The reaction was stirred at room temperature for 5 minutes before a solution of 3-(tert-butoxycarbonylamino)-6-(1-cyanopent-4-enyl)-5-(trifluoromethyl)pyridine-2-carboxylic acid (1.2 g, 3.0048 mmol) in DCM (4.7 mL) was added. The reaction was stirred at room temperature for 1 hour. The reaction was then concentrated under vacuum and purified by silica gel chromatography using a gradient from 0% to 20% ethyl acetate in hexane to furnish tert-butyl N-[6-(1-cyanopent-4-enyl)-2-[5-[2,2,2-trifluoro-1-hydroxy-1-(3-iodophenyl)ethyl]-1,3,4-oxadiazol-2-yl]-5-(trifluoromethyl)-3-pyridyl]carbamate (1.21 g, 50%) as an off-white solid. ESI-MS m/z calc. 723.0777, found 724.1 (M+1)⁺; Retention time: 4.19 minutes (LC Method G).

### Step 2: tert-Butyl N-[16-cyano-6-hydroxy-6,18-bis(trifluoromethyl)-23-oxa-3,4,21-triazatetracyclo[15.3.1.1\hat{ }{2,5}0.1\hat{ }{7,11}]tricosa-1(21),2,4,7,9,11(22),12,17,19-nonaen-20-yl]carbamate (Mixture of Isomers)

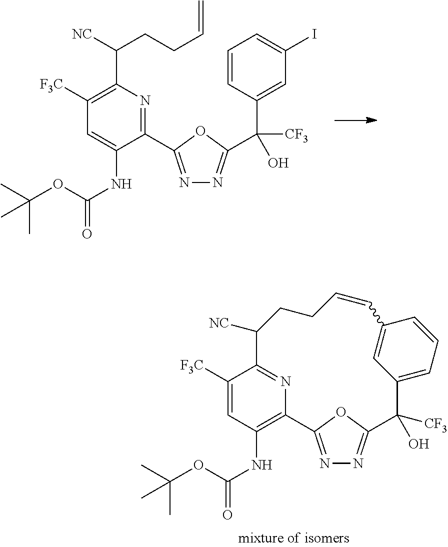 [SMILES: C=CCCC(C#N)c1nc(-c2nnc(C(O)(c3cccc(I)c3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(O)(C(F)(F)F)c1cccc(c1)C=CCCC2C#N]

To a solution of tert-butyl N-[6-(1-cyanopent-4-enyl)-2-[5-[2,2,2-trifluoro-1-hydroxy-1-(3-iodophenyl)ethyl]-1,3,4-oxadiazol-2-yl]-5-(trifluoromethyl)-3-pyridyl]carbamate (34 mg, 0.0470 mmol) in acetonitrile (5 mL) was added tri-o-tolylphosphine (3 mg, 0.0099 mmol) and TEA (23.958 mg, 0.033 mL, 0.2368 mmol). The reaction was purged with argon for 5 minutes. Pd(OAc)₂ (1 mg, 0.0045 mmol) was added to the reaction mixture. The reaction was purged with argon for another 2 minutes. The vial was sealed and heated to 80° C. overnight. The reaction mixture was concentrated under vacuum. The residue was purified by silica gel chromatography using a gradient from 0% to 20% ethyl acetate in hexanes to furnish as a complex mixture, tert-butyl N-[16-cyano-6-hydroxy-6,18-bis(trifluoromethyl)-23-oxa-3,4,21-triazatetracyclo[15.3.1.1\hat{ }{2,5}0.1\hat{ }{7,11}]tricosa-1(21),2,4,7,9,11(22),12,17,19-nonaen-20-yl]carbamate (mixture of isomers) (26 mg, 93%) as an orange solid. ESI-MS m/z calc. 595.1654, found 596.4 (M+1)⁺; Retention time: 3.84 minutes (LC Method G).

### Step 3: tert-Butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 1), tert-butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 2) and tert-butyl N-[15-cyano-6-hydroxy-12-methyl-6,17-bis(trifluoromethyl)-22-oxa-3,4,20-triazatetracyclo[14.3.1.1\hat{ }{2,5}0.1\hat{ }{7,11}]docosa-1(20),2,4,7,9,11(21),16,18-octaen-19-yl]carbamate (Single Diastereomer Pair)

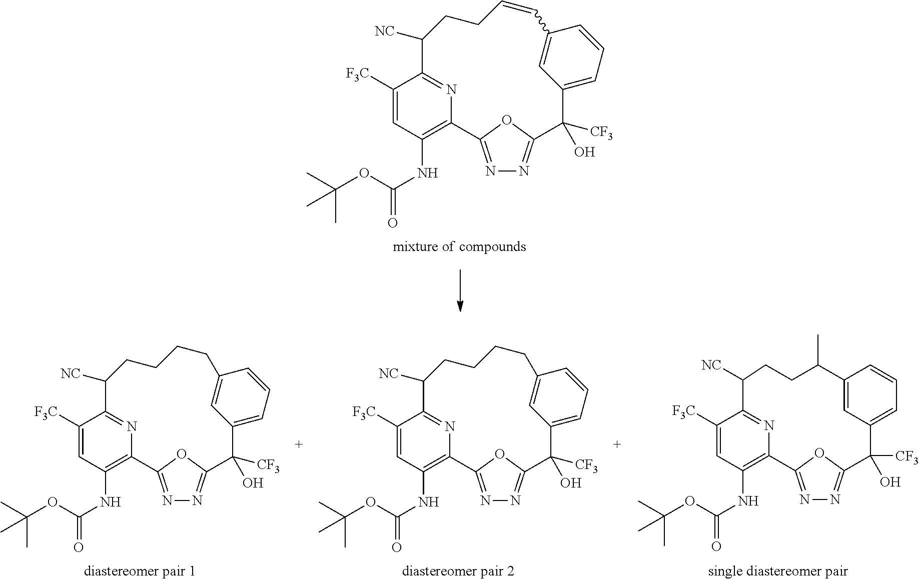 [SMILES: CC(C)(C)OC(=O)Nc1cc(C(F)(F)F)c2nc1C1=NN=C(O1)C(O)(C(F)(F)F)c1cccc(c1)C=CCCC2C#N ; CC(C)(C)OC(=O)Nc1cc(C(F)(F)F)c2nc1C1=NN=C(O1)C(O)(C(F)(F)F)c1cccc(c1)CCCCC2C#N ; CC(C)(C)OC(=O)Nc1cc(C(F)(F)F)c2nc1C1=NN=C(O1)C(O)(C(F)(F)F)c1cccc(c1)CCCCC2C#N ; CC1CCC(C#N)c2nc(c(NC(=O)OC(C)(C)C)cc2C(F)(F)F)C2=NN=C(O2)C(O)(C(F)(F)F)c2cccc1c2]

Into a solution of tert-butyl N-[16-cyano-6-hydroxy-6,18-bis(trifluoromethyl)-23-oxa-3,4,21-triazatetracyclo[15.3.1.1\hat{ }{2,5}0.1\hat{ }{7,11}]tricosa-1(21),2,4,7,9,11(22),12,17,19-nonaen-20-yl]carbamate (mixture of isomers) (623 mg, 1.0043 mmol) in methanol (50 mL) was added 10% Pd/C (200 mg, 0.1879 mmol). The reaction was hydrogenated at 1 atm of H₂ for 16 hours. The catalyst was removed by filtration through a pad of Celite. The filtrate was concentrated, and the residue was purified by silica gel chromatography using a gradient from 0% to 20% ethyl acetate in hexane to furnish three peaks in order of elution:

Peak 1: tert-Butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 1), ESI-MS m/z calc. 597.1811, found 598.1 (M+1)⁺; Retention time: 6.99 minutes and 7.16 minutes (LC Method DD).

Peak 2: tert-Butyl N-[16-cyano-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 2), ESI-MS m/z calc. 597.1811, found 598.3 (M+1)⁺; Retention time: 6.98 minutes and 6.89 minutes (LC Method DD).

Peak 3: tert-Butyl N-[15-cyano-6-hydroxy-12-methyl-6,17-bis(trifluoromethyl)-22-oxa-3,4,20-triazatetracyclo[14.3.1.1\hat{ }{2,5}0.1\hat{ }{7,11}]docosa-1(20),2,4,7,9,11(21),16,18-octaen-19-yl]carbamate (single diastereomer pair), ESI-MS m/z calc. 597.1811, found 598.1 (M+1)⁺; Retention time: 6.91 minutes (LC Method DD).

### Step 4: 19-Amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (Single Diastereomer Pair)

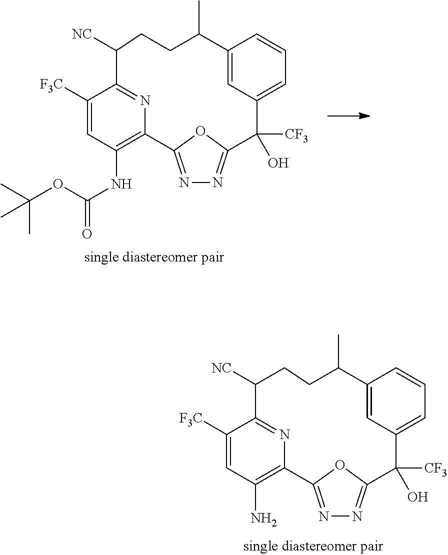 [SMILES: CC1CCC(C#N)c2nc(c(NC(=O)OC(C)(C)C)cc2C(F)(F)F)C2=NN=C(O2)C(O)(C(F)(F)F)c2cccc1c2 ; CC1CCC(C#N)c2nc(c(N)cc2C(F)(F)F)C2=NN=C(O2)C(O)(C(F)(F)F)c2cccc1c2]

Into a solution of tert-butyl N-[15-cyano-6-hydroxy-12-methyl-6,17-bis(trifluoromethyl)-22-oxa-3,4,20-triazatetracyclo[14.3.1.1\hat{ }{2,5}0.1\hat{ }{7,11}]docosa-1(20),2,4,7,9,11(21),16,18-octaen-19-yl]carbamate (single diastereomer pair) (108 mg, 0.1789 mmol) in DCM (10 mL) was added 4 M HCl in dioxane (10 mL of 4 M, 40 mmol). The reaction was stirred at room temperature for 16 hours. The reaction was diluted with saturated sodium bicarbonate (50 mL) and ethyl acetate (50 mL). Two layers were separated, and the aqueous layer was extracted with ethyl acetate (2×50 mL). The combined organic layers were washed with brine (50 mL), dried over anhydrous sodium sulfate, and concentrated under vacuum. The residue was purified by reverse phase HPLC using 0% to 100% acetonitrile in water (+0.1% TFA). The pure fractions were combined and neutralized with saturated NaHCO₃ (30 mL). The solution was extracted with ethyl acetate (3×30 mL). The combined organic layers were washed with brine (30 mL), dried over anhydrous sodium sulfate, and concentrated under vacuum to provide as a white solid, 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (single diastereomer pair) (67.2 mg, 71%). ¹H NMR (500 MHz, DMSO-d₆) δ 8.52 (s, 1H), 7.74 (s, 1H), 7.71 (s, 1H), 7.63 (m, 1H), 7.37 (t, J=7.7, 7.7 Hz, 1H), 7.31 (m, 1H), 6.89 (s, 2H), 4.63 (d, J=5.9 Hz, 1H), 2.84-2.72 (m, 1H), 2.44-2.32 (m, 1H), 1.96-1.87 (m, 1H), 1.84-1.70 (m, 2H), 1.23 (d, J=6.8 Hz, 3H) ppm. ESI-MS m/z calc. 497.1286, found 498.4 (M+1)⁺; Retention time: 2.81 minutes (LC Method H).

### Step 5: 19-Amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (enantiomer 1) (Compound 65) and 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (Enantiomer 2) (Compound 66)

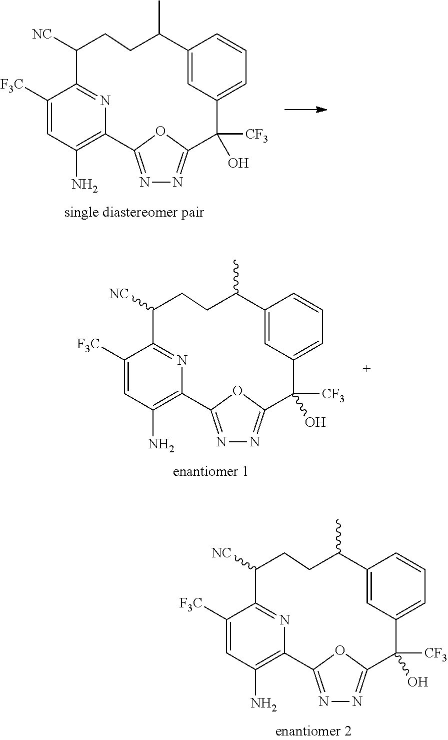

19-Amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (single diastereomer pair) (62.6 mg, 0.1259 mmol) was separated using a normal phase SFC-MS method using a LUX-4 column (250×21.2 mm, 5 m particle size) sold by Phenomenex (pn: 00G-4491-P0-AX), and a dual gradient run from 20% to 30% MeOH (+20 mM NH₃) in CO₂ with a flow rate of 40 mL/min over 14.5 minutes which provided as a white solid and the first product to elute, 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (enantiomer 1) (9.1 mg, 26%). ¹H NMR (400 MHz, DMSO-d₆) δ 8.53 (s, 1H), 7.75 (s, 1H), 7.72 (s, 1H), 7.64 (d, J=7.8 Hz, 1H), 7.37 (t, J=7.6 Hz, 1H), 7.31 (d, J=7.5 Hz, 1H), 6.90 (s, 2H), 4.63 (d, J=5.5 Hz, 1H), 2.79 (p, J=6.7 Hz, 1H), 2.39 (q, J=11.7 Hz, 1H), 1.91 (d, J=13.1 Hz, 1H), 1.79 (t, J=11.0 Hz, 2H), 1.24 (d, J=6.9 Hz, 3H) ppm. ¹⁹F NMR (376 MHz, DMSO-d₆) δ−58.76, −75.92 ppm. ESI-MS m/z calc. 497.12863, found 498.0 (M+1)⁺; Retention time: 2.58 minutes (LC Method D).

The second product to elute was isolated as a white solid, 19-amino-6-hydroxy-12-methyl-6,17-bis(trifluoromethyl)-22-oxa-3,4,20-triazatetracyclo[14.3.1.12,5.17,11]docosa-1(19),2,4,7(21),8,10,16(20),17-octaene-15-carbonitrile (enantiomer 2) (8.5 mg, 24%). ESI-MS m/z calc. 497.12863, found 498.0 (M+1)⁺; Retention time: 2.58 minutes (LC Method D).

### Example 38: Preparation of 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2, enantiomer 1) (Compound 67), 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2, enantiomer 2) (Compound 68) and 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (Diastereomer Pair 1) (Compound 69)

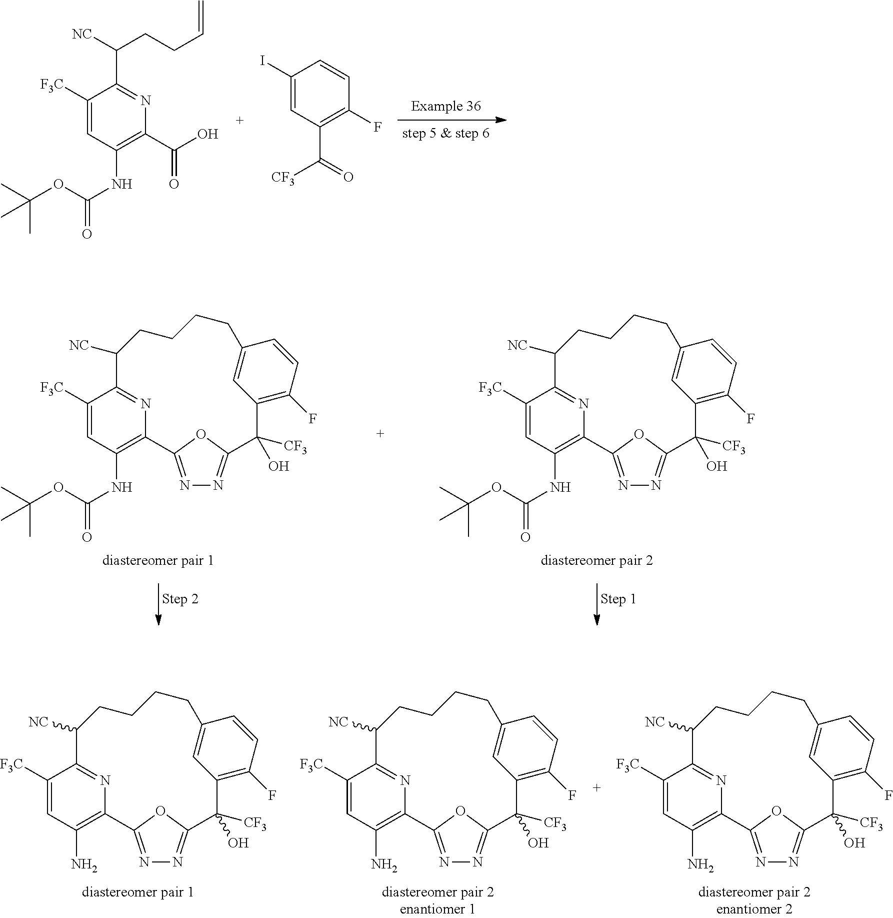

tert-Butyl N-[16-cyano-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 1) and tert-butyl N-[16-cyano-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 2) were prepared using 3-(tert-butoxycarbonylamino)-6-(1-cyanopent-4-enyl)-5-(trifluoromethyl)pyridine-2-carboxylic acid (510 mg, 1.277 mmol) and 2,2,2-trifluoro-1-(2-fluoro-5-iodo-phenyl)ethanone (1.01 g, 3.176 mmol) in a manner analogous to the procedures described in steps 5 and 6 in Example 36.

### Step 1: 20-Amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2, enantiomer 1) (Compound 67) and 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (Diastereomer Pair 2, Enantiomer 2) (Compound 68)

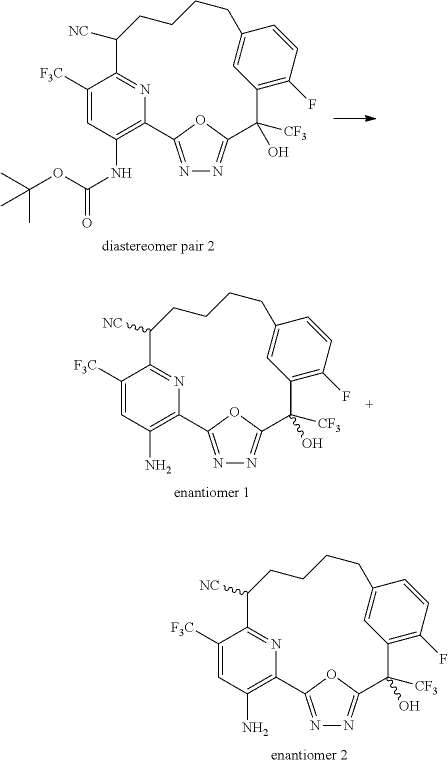 [SMILES: CC(C)(C)OC(=O)Nc1cc(C(F)(F)F)c2nc1C1=NN=C(O1)C(O)(C(F)(F)F)c1cc(ccc1F)CCCCC2C#N ; N#CC1CCCCc2ccc(F)c(c2)C(O)(C(F)(F)F)C2=NN=C(O2)c2nc1c(C(F)(F)F)cc2N ; N#CC1CCCCc2ccc(F)c(c2)C(O)(C(F)(F)F)C2=NN=C(O2)c2nc1c(C(F)(F)F)cc2N]

tert-Butyl N-[16-cyano-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 2) (15 mg, 0.02437 mmol) in dichloromethane (2 mL) was treated with TFA (1.5 mL, 19.47 mmol) analogous to the procedure described in step 7 of Example 36. The residue was purified by normal phase chiral SFC using an IG column (250×21.2 mm, 5 μm particle size) sold by Chiral Technologies and a dual gradient run from 50% to 80% MeOH (20 mM NH₃) and CO₂ with a flow rate of 40 mL/min over 14.5 minutes which gave as a white solid and the first eluting enantiomer, 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2, enantiomer 1) (3.6 mg, 28%). ¹H NMR (400 MHz, CDCl₃) δ 7.37 (d, J=5.1 Hz, 2H), 7.33-7.20 (m, 1H), 7.12-6.98 (m, 1H), 6.00 (s, 2H), 5.04 (s, 1H), 4.33 (dd, J=5.5, 2.4 Hz, 1H), 2.74 (m, 1H), 2.07-1.93 (m, 1H), 1.92-1.80 (m, 2H), 1.76-156 (m, 4H) ppm. ESI-MS m/z calc. 515.1192, found 516.1 (M+1)⁺; Retention time: 1.31 minutes (LC Method J).

The later eluting enantiomer was isolated as a white solid, 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 2, enantiomer 2) (1.8 mg, 14%). (ESI-MS m/z calc. 515.1192, found 516.1 (M+1)⁺; Retention time: 1.31 minutes (LC Method J).

### Step 2: 20-Amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (Diastereomer Pair 1) (Compound 69)

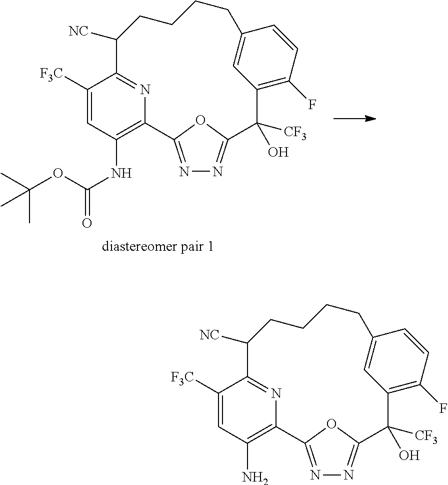 [SMILES: CC(C)(C)OC(=O)Nc1cc(C(F)(F)F)c2nc1C1=NN=C(O1)C(O)(C(F)(F)F)c1cc(ccc1F)CCCCC2C#N ; N#CC1CCCCc2ccc(F)c(c2)C(O)(C(F)(F)F)C2=NN=C(O2)c2nc1c(C(F)(F)F)cc2N]

tert-Butyl N-[16-cyano-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaen-20-yl]carbamate (diastereomer pair 1) (7 mg, 0.0114 mmol) in dichloromethane (1.5 mL) was treated with TFA (1 mL, 12.5 mmol) analogous to the procedure described in step 7 of Example 36 which gave as a white solid, 20-amino-8-fluoro-6-hydroxy-6,18-bis(trifluoromethyl)-23-oxa-3,4,21-triazatetracyclo[15.3.1.12,5.17,11]tricosa-1(21),2,4,7,9,11(22),17,19-octaene-16-carbonitrile (diastereomer pair 1) (2.5 mg, 41.81%). ESI-MS m/z calc. 515.1192, found 516.0 (M+1)⁺; Retention time: 1.27 minutes (LC Method J).

### Example 39: Preparation of 17-amino-6-fluoro-6,15-bis(trifluoromethyl)-19-oxa-3,4,18-triazatricyclo[12.3.1.12,5]nonadeca-1(18),2,4,14,16-pentaen-13-one (enantiomer 1) (Compound 70) and 17-amino-6-fluoro-6,15-bis(trifluoromethyl)-19-oxa-3,4,18-triazatricyclo[12.3.1.12,5]nonadeca-1(18),2,4,14,16-pentaen-13-one (Enantiomer 2) (Compound 71)

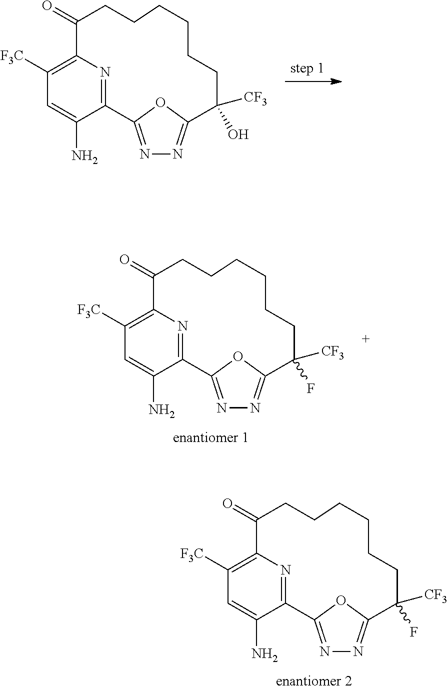 [SMILES: Nc1cc(C(F)(F)F)c2nc1C1=NN=C(O1)[C@](O)(C(F)(F)F)CCCCCCC2=O ; Nc1cc(C(F)(F)F)c2nc1C1=NN=C(O1)C(F)(C(F)(F)F)CCCCCCC2=O ; Nc1cc(C(F)(F)F)c2nc1C1=NN=C(O1)C(F)(C(F)(F)F)CCCCCCC2=O]

### Step 1: 17-Amino-6-fluoro-6,15-bis(trifluoromethyl)-19-oxa-3,4,18-triazatricyclo[12.3.1.12,5]nonadeca-1(18),2,4,14,16-pentaen-13-one (enantiomer 1) (Compound 70) and 17-amino-6-fluoro-6,15-bis(trifluoromethyl)-19-oxa-3,4,18-triazatricyclo[12.3.1.12,5]nonadeca-1(18),2,4,14,16-pentaen-13-one (Enantiomer 2) (Compound 71)

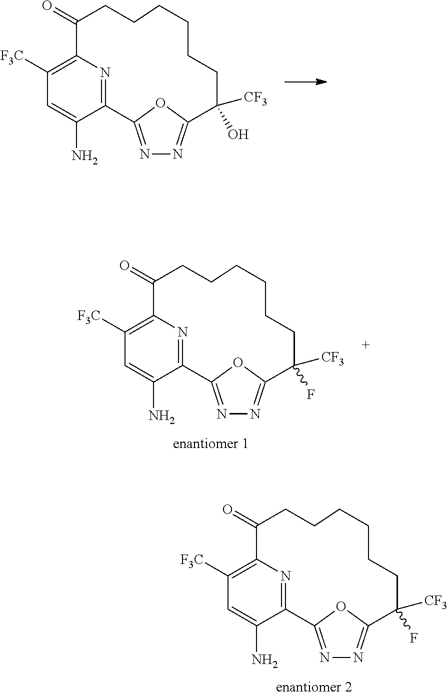 [SMILES: Nc1cc(C(F)(F)F)c2nc1C1=NN=C(O1)[C@](O)(C(F)(F)F)CCCCCCC2=O ; Nc1cc(C(F)(F)F)c2nc1C1=NN=C(O1)C(F)(C(F)(F)F)CCCCCCC2=O ; Nc1cc(C(F)(F)F)c2nc1C1=NN=C(O1)C(F)(C(F)(F)F)CCCCCCC2=O]

A stirred solution of (6S)-17-amino-6-hydroxy-6,15-bis(trifluoromethyl)-19-oxa-3,4,18-triazatricyclo[12.3.1.12,5]nonadeca-1(18),2,4,14,16-pentaen-13-one (10 mg, 0.02281 mmol) in dichloromethane (1.5 mL) was cooled to 0° C. in an ice-bath, then to the solution was added bis(2-methoxyethyl)aminosulfur trifluoride (20.19 mg of 50% w/w, 0.04563 mmol) dropwise. The reaction mixture was warmed to room temperature and stirred for 15 min. The mixture was then quenched with saturated aqueous sodium bicarbonate and extracted with dichloromethane (2×15 mL). The organic layers were combined and washed with brine, dried over sodium sulfate, filtered, and concentrated to a white solid. The mixture was subjected to SFC separation using the OD column (250×20 mm; 5 μm), and 12% MeOH (+20 mM ammonia), 88% CO₂ at a flow rate of 70 mL/min to provide as the first product to elute, 17-amino-6-fluoro-6,15-bis(trifluoromethyl)-19-oxa-3,4,18-triazatricyclo[12.3.1.12,5]nonadeca-1(18),2,4,14,16-pentaen-13-one (enantiomer 1) (3 mg, 30%). ¹H NMR (400 MHz, methanol-d4) δ 7.63 (s, 1H), 2.88 (m, 1H), 2.75 (m, 1H), 2.45 (m, 2H), 2.12-1.96 (m, 1H), 1.80 (q, J=6.0, 5.5 Hz, 1H), 1.75-1.46 (m, 5H) ppm. One proton obscured by solvent peak. ESI-MS m/z calc. 440.10834, found 441.3 (M+1)⁺; Retention time: 2.04 minutes (LC Method JJ).

The second product to elute was isolated as 17-amino-6-fluoro-6,15-bis(trifluoromethyl)-19-oxa-3,4,18-triazatricyclo[12.3.1.12,5]nonadeca-1(18),2,4,14,16-pentaen-13-one (enantiomer 2) (3 mg, 30%). ¹H NMR (400 MHz, methanol-d4) δ 7.74 (d, J=6.3 Hz, 1H), 3.37 (d, J=3.7 Hz, 1H), 3.06-2.77 (m, 2H), 2.68-2.34 (m, 2H), 2.14 (s, 1H), 1.97-1.58 (m, 6H) ppm. ESI-MS m/z calc. 440.10834, found 441.2 (M+1)⁺; Retention time: 2.04 minutes (LC Method JJ).

### Method 13: Final Product Synthesis by Ring-Closing Metathesis Route

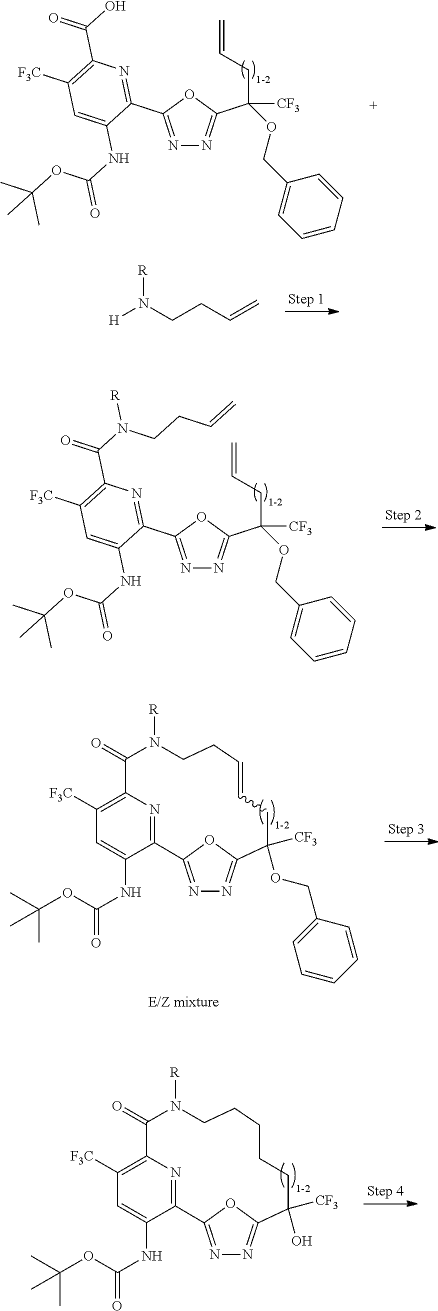

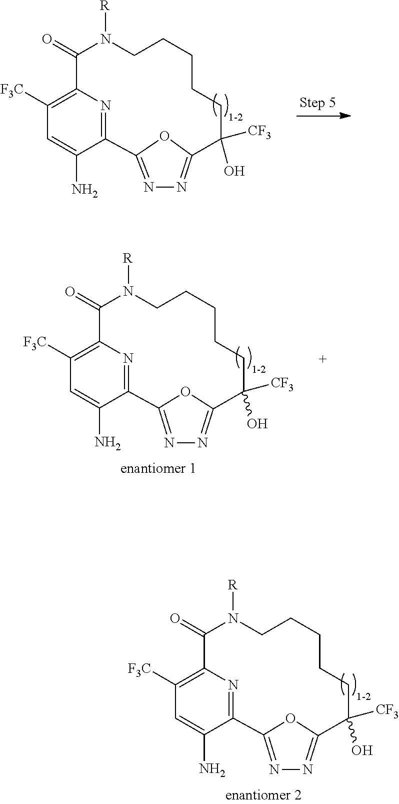

### Step 1: Amide Coupling

To a solution of 6-[5-[1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid, 6-[5-[(1R)-1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid or 6-[5-[(1R)-1-benzyloxy-1-(trifluoromethyl)pent-4-enyl]-1,3,4-oxadiazol-2-yl]-5-(tert-butoxycarbonylamino)-3-(trifluoromethyl)pyridine-2-carboxylic acid (1 equivalent) in DMF or THE (20 volume equivalents) was added N-substituted butenyl amine (3 equivalents), a coupling agent such as HATU (2 to 5 equivalents) or T3P (5 to 10 eq of 50% w/w solution in ethyl acetate) and then a base such as Et₃N (5 to 10 equivalents) or DIEA (5 to 10 equivalents) was added. The mixture was stirred at room temperature and the reaction progress was monitored by LCMS. After the starting material was consumed, the reaction mixture was diluted with diethyl ether or ethyl acetate, washed with water, dried (MgSO₄), filtered and evaporated. The residue was purified by silica gel chromatography using ethyl acetate and hexanes which gave the desired bis-olefin amide products.

### Step 2: Macrocyclization

To a two-neck flask with constant nitrogen bubbling through the solution was added bis-olefin (1 equivalent) in 1,2-dichloroethane (200 to 2500 volume equivalents) and the solution was heated to 50 to 60° C. Then added dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (˜0.25 to 0.4 equivalents) in 1,2-dichloroethane (˜60 volume equivalents) via syringe and the mixture was stirred at 50 to 70° C. for several hours while monitoring the reaction progress by LCMS. After most of the starting material was consumed, the reaction mixture was evaporated under reduced pressure. The residue was purified by silica gel chromatography using ethyl acetate and hexanes which gave the macrocyclization product as E/Z mixture that was taken directly to the next reaction.

### Step 3: Hydrogenation

To a solution of E/Z mixture of macrocycle (1 equivalent) in acetic acid (˜25 volume equivalents) was added Pd/C (˜0.5 eq of 10% w/w). The mixture was subjected to 200 psi hydrogen atmosphere in a stainless steel pressure vessel while monitoring the reaction progress for both olefin hydrogenation and de-benzylation by LCMS. After completion of the reaction, the mixture was filtered through Celite, washing well with ethyl acetate and then the filtrate was concentrated. The residue was purified by reverse phase chromatography using acetonitrile and water (+5 mM HCl) which provided the desired product.

### Step 4: Removal of Boc Group

To a solution of the Boc-containing macrocycle (1 equivalent) in a solvent such as DCM or water (˜20 volume equivalents) was added TFA (˜20-200 equivalents) and triisopropylsilane (2 equivalents) and the resulting mixture was stirred at room temperature for several hours while monitoring the reaction progress by LCMS. After the starting material was consumed, the solvents were evaporated and the residue was dissolved in DCM and washed with saturated aqueous NaHCO₃, brine, dried over Na₂SO₄, filtered, and concentrated to give the desired final product which, if needed, was further purified by C₁₈ reverse phase HPLC using acetonitrile and water with HCl modifier.

### Step 5: Chiral Separation

Racemic compounds were purified by chiral SFC using a suitable chiral column and mobile phase as indicated in Table 4 which gave as the first eluting enantiomer, enantiomer 1 and the later eluting enantiomer, enantiomer 2.

### Representative Synthesis of Final Compounds by Method 13 (Ring-Closing Metathesis Route)

### Example 40: Preparation of 17-Amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (enantiomer 1) (Compound 72) and 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (Enantiomer 2) (Compound 73)

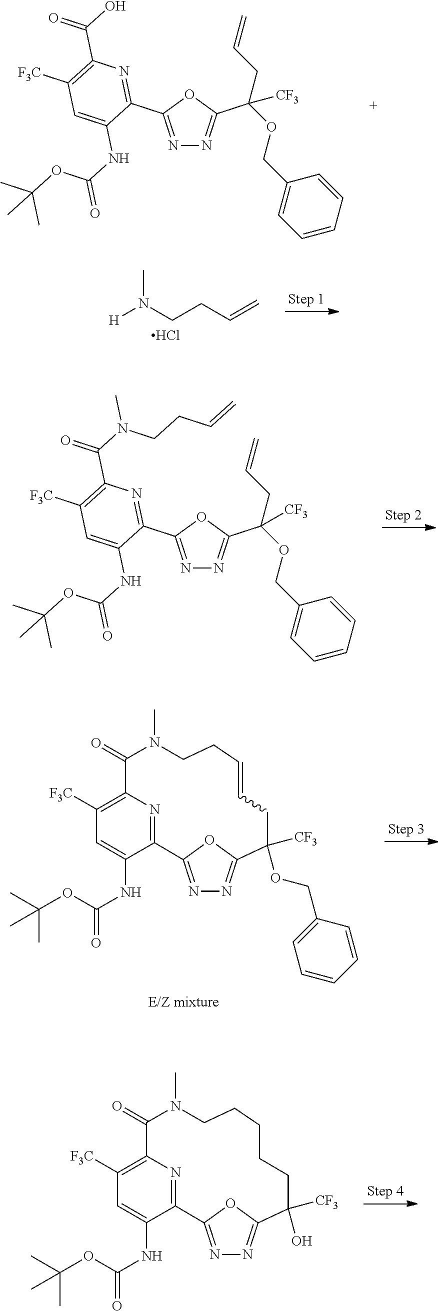

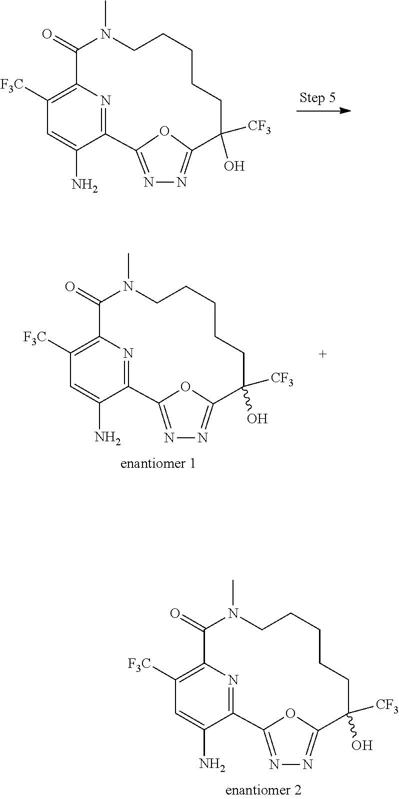 [SMILES: CN1CCCCCC(O)(C(F)(F)F)C2=NN=C(O2)c2nc(c(C(F)(F)F)cc2N)C1=O ; CN1CCCCCC(O)(C(F)(F)F)c2nnc(o2)-c2nc(c(C(F)(F)F)cc2N)C1=O ; CN1CCCCCC(O)(C(F)(F)F)c2nnc(o2)-c2nc(c(C(F)(F)F)cc2N)C1=O]

### Step 1: tert-Butyl N-[2-[5-[1-Benzyloxy-1-(trifluoromethyl)but-3-enyl]-1,3,4-oxadiazol-2-yl]-6-[but-3-enyl(methyl)carbamoyl]-5-(trifluoromethyl)-3-pyridyl]carbamate

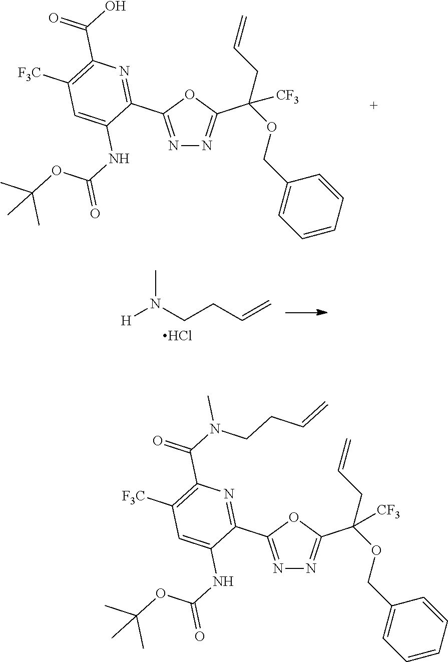

DMF (3 mL) was added to 6-[5-[1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid (147 mg, 0.2440 mmol), N-methylbut-3-en-1-amine (hydrochloride salt) (91 mg, 0.7483 mmol) and HATU (182 mg, 0.4787 mmol) and then DIEA (255 μL, 1.464 mmol) was added. The mixture was stirred at room temperature for 30 min, then diluted with ether, washed with water, dried (MgSO₄), filtered and evaporated. The residue was purified by silica gel chromatography using a gradient from 0% to 10% ethyl acetate in hexanes over 15 min which provided tert-butyl N-[2-[5-[1-benzyloxy-1-(trifluoromethyl)but-3-enyl]-1,3,4-oxadiazol-2-yl]-6-[but-3-enyl(methyl)carbamoyl]-5-(trifluoromethyl)-3-pyridyl]carbamate (111 mg, 68%). ESI-MS m/z calc. 669.2386, found 670.2 (M+1)⁺; Retention time: 0.87 minutes (LC Method S).

### Step 2: tert-Butyl N-[6-benzyloxy-12-methyl-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z Mixture)

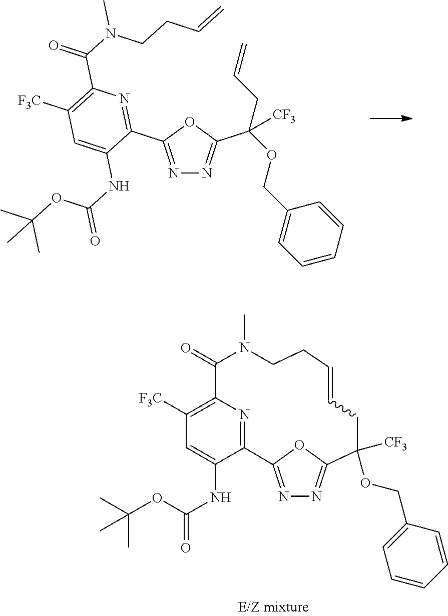 [SMILES: C=CCCN(C)C(=O)c1nc(-c2nnc(C(CC=C)(OCc3ccccc3)C(F)(F)F)o2)c(NC(=O)OC(C)(C)C)cc1C(F)(F)F ; CN1CCC=CCC(OCc2ccccc2)(C(F)(F)F)c2nnc(o2)-c2nc(c(C(F)(F)F)cc2NC(=O)OC(C)(C)C)C1=O]

To a two neck flask with constant nitrogen bubbling through the solution was added tert-butyl N-[2-[5-[1-benzyloxy-1-(trifluoromethyl)but-3-enyl]-1,3,4-oxadiazol-2-yl]-6-[but-3-enyl(methyl)carbamoyl]-5-(trifluoromethyl)-3-pyridyl]carbamate (108 mg, 0.1613 mmol) in 1,2-dichloroethane (25 mL) and the solution was heated to 50° C. Then, via syringe, was added dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (30 mg, 0.04 mmol) in 1,2-dichloroethane (5 mL), and the mixture stirred at 70° C. for 1 h. The reaction mixture was evaporated under reduced pressure and purified by silica gel chromatography using a gradient from 0% to 30% ethyl acetate in hexanes to provide as a white solid, tert-butyl N-[6-benzyloxy-12-methyl-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (83 mg, 80%). ESI-MS m/z calc. 641.2073, found 642.3 (M+1)⁺; Retention time: 0.79 minutes (LC Method R).

### Step 3: tert-Butyl N-[6-hydroxy-12-methyl-13-oxo-6,15-bis(trifluoromethyl)-19-oxa-3,4,12,18-tetrazatricyclo[12.3.1.12,5]nonadeca-1(17),2,4,14(18),15-pentaen-17-yl]carbamate

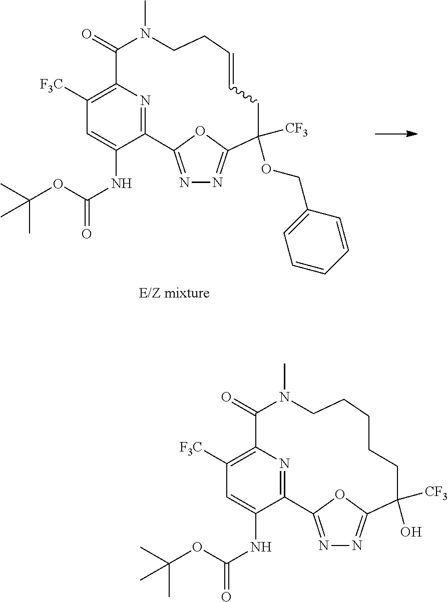 [SMILES: CN1CCC=CCC(OCc2ccccc2)(C(F)(F)F)c2nnc(o2)-c2nc(c(C(F)(F)F)cc2NC(=O)OC(C)(C)C)C1=O ; CN1CCCCCC(O)(C(F)(F)F)c2nnc(o2)-c2nc(c(C(F)(F)F)cc2NC(=O)OC(C)(C)C)C1=O]

To a solution of tert-butyl N-[6-benzyloxy-12-methyl-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (80 mg, 0.1247 mmol) in acetic acid (2.16 mL) was added Pd/C (53 mg of 10% w/w, 0.05 mmol). The mixture was stirred for 16 h under 200 psi hydrogen atmosphere in a stainless steel pressure vessel. The reaction mixture was filtered through a silica plug, washing well with ethyl acetate and then the filtrate was concentrated to provide as a white solid, tert-butyl N-[6-hydroxy-12-methyl-13-oxo-6,15-bis(trifluoromethyl)-19-oxa-3,4,12,18-tetrazatricyclo[12.3.1.12,5]nonadeca-1(17),2,4,14(18),15-pentaen-17-yl]carbamate (60 mg, 87%). ESI-MS m/z calc. 553.17596, found 554.3 (M+1)⁺; Retention time: 0.54 minutes (LC Method R).

### Step 4: 17-Amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one

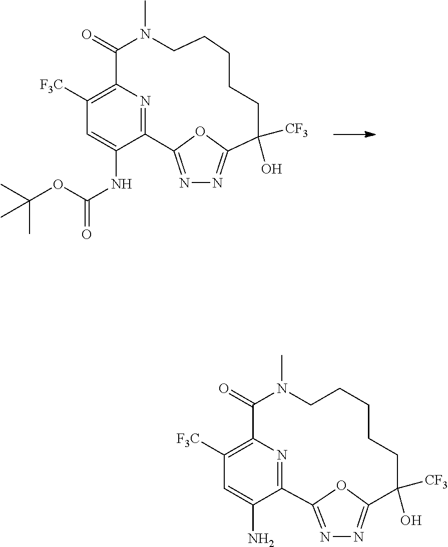 [SMILES: CN1CCCCCC(O)(C(F)(F)F)c2nnc(o2)-c2nc(c(C(F)(F)F)cc2NC(=O)OC(C)(C)C)C1=O ; CN1CCCCCC(O)(C(F)(F)F)C2=NN=C(O2)c2nc(c(C(F)(F)F)cc2N)C1=O]

tert-Butyl N-[6-hydroxy-12-methyl-13-oxo-6,15-bis(trifluoromethyl)-19-oxa-3,4,12,18-tetrazatricyclo[12.3.1.12,5]nonadeca-1(17),2,4,14(18),15-pentaen-17-yl]carbamate (60 mg, 0.108 mmol) was dissolved in DCM (1 mL) and to the mixture was added TFA (417 μL, 5.41 mmol) and the mixture was stirred at room temperature for 2 h. The solvents were evaporated then the residue was dissolved into DCM and washed with saturated aqueous NaHCO₃. The organic layer was concentrated to provide as a white solid, racemic 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one which was used directly in the next step.

### Step 5: 17-Amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (enantiomer 1) (Compound 72) and 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (Enantiomer 2) (Compound 73)

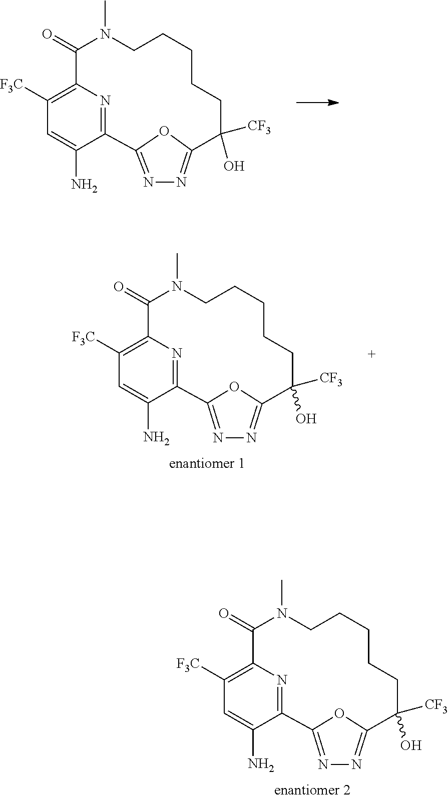 [SMILES: CN1CCCCCC(O)(C(F)(F)F)C2=NN=C(O2)c2nc(c(C(F)(F)F)cc2N)C1=O ; CN1CCCCCC(O)(C(F)(F)F)c2nnc(o2)-c2nc(c(C(F)(F)F)cc2N)C1=O ; CN1CCCCCC(O)(C(F)(F)F)c2nnc(o2)-c2nc(c(C(F)(F)F)cc2N)C1=O]

Racemic 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one was purified by chiral SFC using an AS-3 column (250×21.2 mm, 5 m particle size) and a gradient of MeOH (+5 mM NH₃) in CO₂ which gave as a white solid and the first eluting enantiomer, 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (enantiomer 1) (12 mg, 49%). ¹H NMR (400 MHz, CDCl₃) δ 7.44 (s, 1H), 5.92 (s, 2H), 4.16 (s, 1H), 3.22 (m, 1H), 3.06 (s, 3H), 2.95 (m, 1H), 2.42 (m, 1H), 2.37-2.27 (m, 1H), 2.22 (m, 1H), 1.91-1.77 (m, 1H), 1.73-1.59 (m, 2H), 1.23-1.14 (m, 1H), 0.76 (m, 1H) ppm. ESI-MS m/z calc. 453.12357, found 454.4 (M+1)⁺; Retention time: 1.57 minutes (LC Method A).

The later eluting enantiomer was isolated as a white solid, 17-amino-6-hydroxy-12-methyl-6,15-bis(trifluoromethyl)-19-oxa-3,4,12,18-tetrazatricyclo[12.3.1.12,5]nonadeca-1(18),2,4,14,16-pentaen-13-one (enantiomer 2) (15 mg, 61%). ¹H NMR (400 MHz, CDCl₃) δ 7.44 (s, 1H), 5.94 (s, 2H), 4.32 (s, 1H), 3.21 (in, 1H), 3.06 (s, 3H), 2.96 (in, 1H), 2.48-2.37 (in, 1H), 2.36-2.27 (in, 1H), 2.22 (in, 1H), 1.94-1.78 (in, 1H), 1.65 (q, J=5.7, 4.8 Hz, 2H), 1.24-1.14 (m, 1H), 0.78 (in, 1H) ppm. ESI-MS m/z calc. 453.12357, found 454.2 (M+1)⁺; Retention time: 1.56 minutes (LC Method A).

TABLE 3
Final Examples Prepared by Method 13 and Analogous to the
Representative Procedure Described for Example 40

| | Amine Structure | |
|---|---|---|
| | Method of | |
| | Amine | |
| | Preparation (if | |
| Example | synthesized) | Carboxylic Acid |
| number | Mass Spec data | Structure |
| 41 | 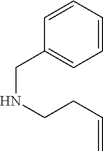 | 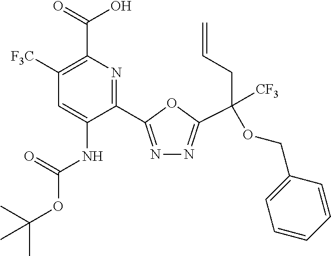 [SMILES: C=CCC(OCc1ccccc1)(c1nnc(-c2nc(C(=O)O)c(C(F)(F)F)cc2NC(=O)OC(C)(C)C)o1)C(F)(F)F] |
| 42 | 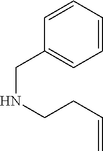 | 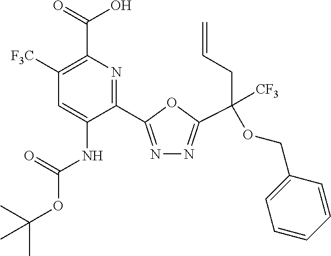 [SMILES: C=CCC(OCc1ccccc1)(c1nnc(-c2nc(C(=O)O)c(C(F)(F)F)cc2NC(=O)OC(C)(C)C)o1)C(F)(F)F] |
| 43 | 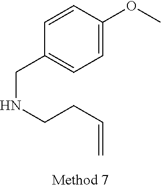 ESI-MS m/z calc. 191.13101, found 192.1 (M + 1)⁺ | 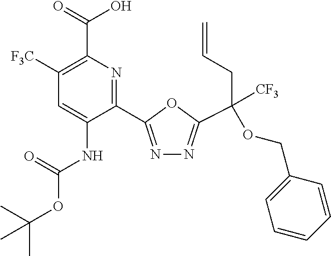 [SMILES: C=CCC(OCc1ccccc1)(c1nnc(-c2nc(C(=O)O)c(C(F)(F)F)cc2NC(=O)OC(C)(C)C)o1)C(F)(F)F] |
| 44 | 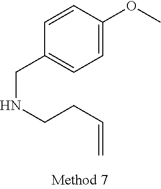 ESI-MS m/z calc. 191.13101, found 192.1 (M + 1)⁺ | 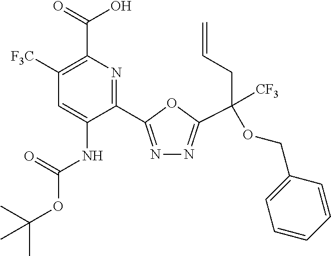 [SMILES: C=CCC(OCc1ccccc1)(c1nnc(-c2nc(C(=O)O)c(C(F)(F)F)cc2NC(=O)OC(C)(C)C)o1)C(F)(F)F] |
| 45 | 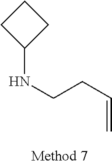 | 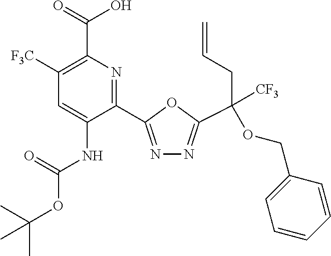 [SMILES: C=CCC(OCc1ccccc1)(c1nnc(-c2nc(C(=O)O)c(C(F)(F)F)cc2NC(=O)OC(C)(C)C)o1)C(F)(F)F] |
| 46 | 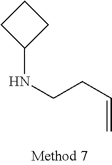 | 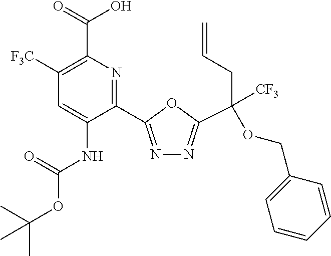 [SMILES: C=CCC(OCc1ccccc1)(c1nnc(-c2nc(C(=O)O)c(C(F)(F)F)cc2NC(=O)OC(C)(C)C)o1)C(F)(F)F] |
| 47 | 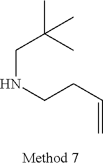 ESI-MS m/z calc. 141.15175, found 142.2 (M + 1)⁺ | 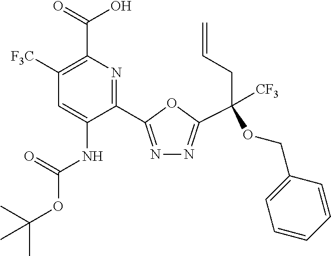 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 48 | 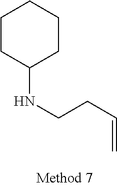 ESI-MS m/z calc. 153.15175, found 154.1 (M + 1)⁺ | 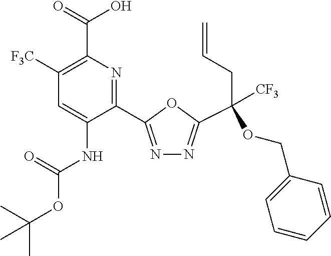 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 49 | 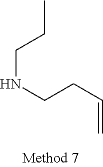 ESI-MS m/z calc. 113.12045, found 114.1 (M + 1)⁺ | 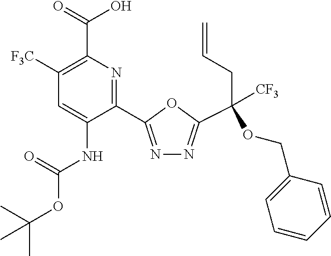 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 50 | 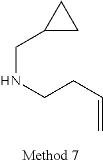 ESI-MS m/z calc. 125.12045, found 126.1 (M + 1)⁺ | 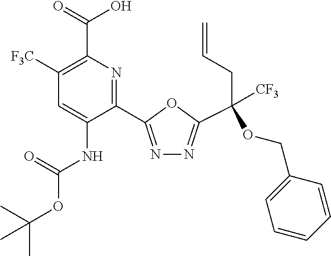 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 51 | 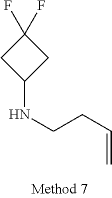 | 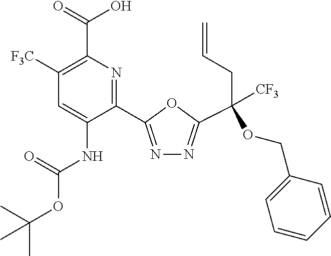 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 52 | 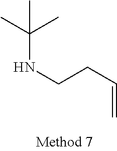 | 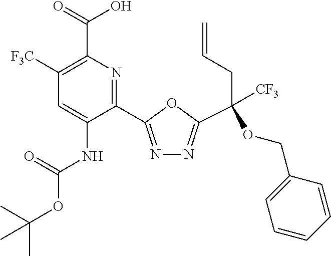 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 53 | 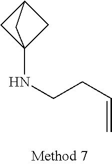 ESI-MS m/z calc. 137.12045, found 138.1 (M + 1)⁺ | 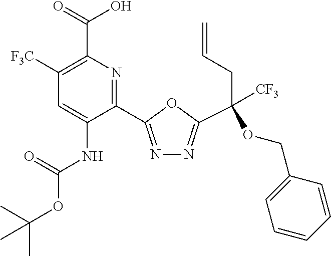 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 54 | 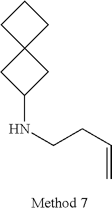 ESI-MS m/z calc. 165.15175, found 166.1 | 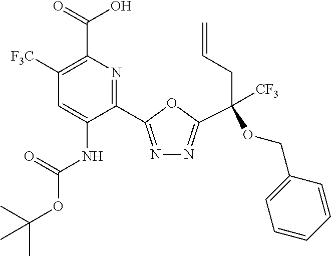 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 55 | 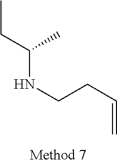 ESI-MS m/z calc. 127.1361, found 128.1 (M + 1)⁺ | 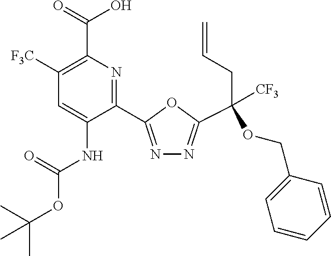 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 56 | 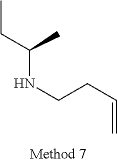 ESI-MS m/z calc. 127.1361, found 128.1 (M + 1)⁺ | 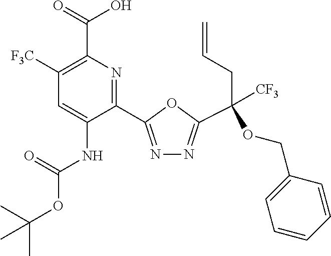 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 57 | 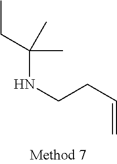 ESI-MS m/z calc. 141.15175, found 142.1 (M + 1)⁺ | 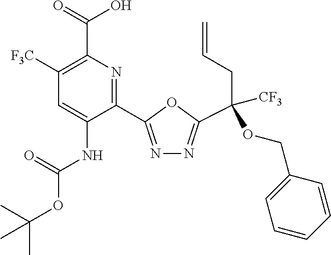 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 58 | 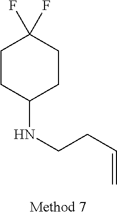 ESI-MS m/z calc. 189.1329, found 190.2 | 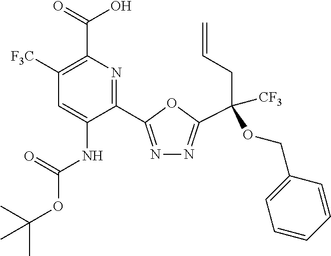 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 59 | 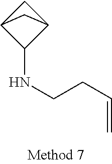 ESI-MS m/z calc. 137.12045, found 138.1 (M + 1)⁺ | 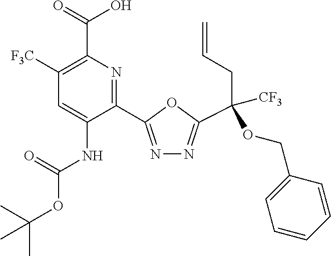 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 60 | 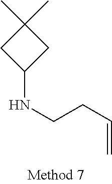 ESI-MS m/z calc. 153.15175, found 154.1 (M + 1)⁺ | 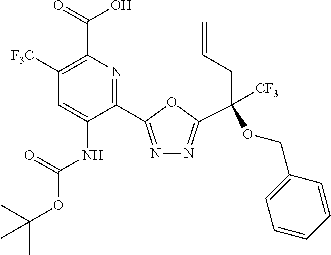 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 61 | 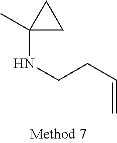 ESI-MS m/z calc. 125.12045, found 126.1 (M + 1)⁺ | 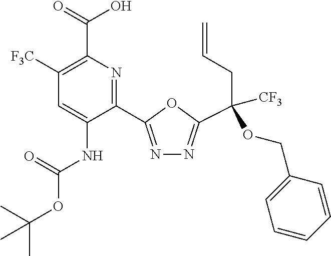 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 62 | 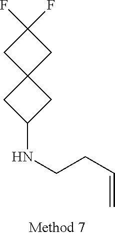 ESI-MS m/z calc. 201.1329, | 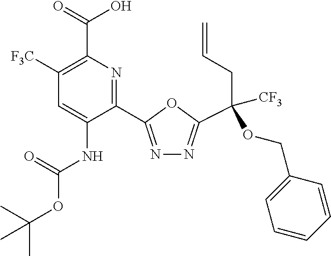 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 202.1 | |
| | (M + 1)⁺ | |
| 63 | 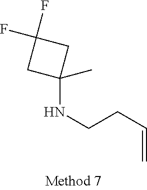 ESI-MS m/z calc. 175.11725, found 176.2 (M + 1)⁺ | 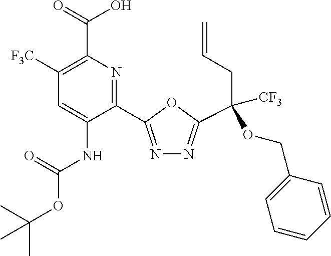 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 64 | 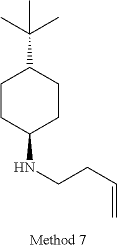 | 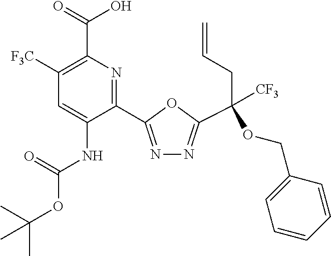 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | ESI-MS m/z | |
| | calc. 209.21436, | |
| | found 210.3 | |
| | (M + 1)⁺ | |
| 65 | 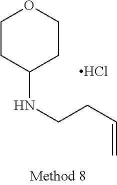 | 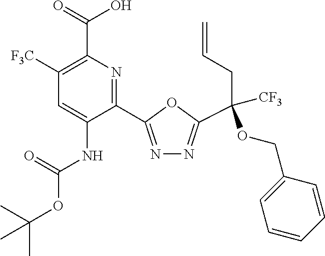 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 66¹ | 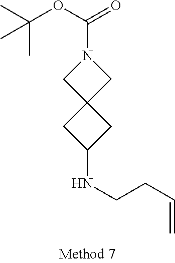 [SMILES: C=CCCNC1CC2(C1)CN(C(=O)OC(C)(C)C)C2] | 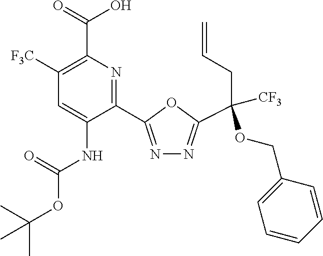 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 67 | 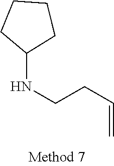 | 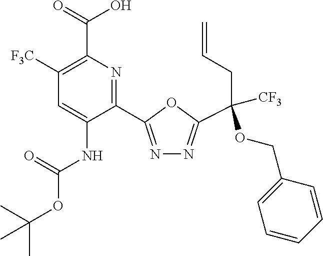 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 68 | 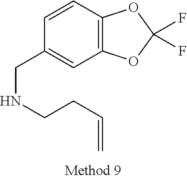 [SMILES: C=CCCNCc1ccc2c(c1)OC(F)(F)O2] ESI-MS m/z calc. 241.09143, found 242.39 (M + 1)⁺ | 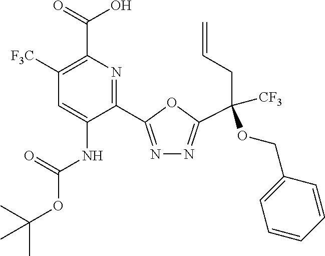 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 69 | 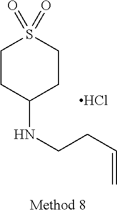 | 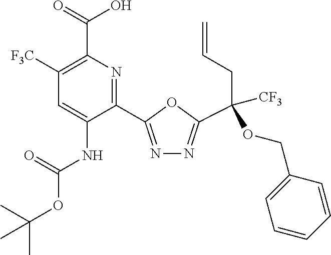 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 70 | 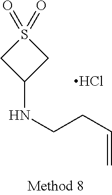 | 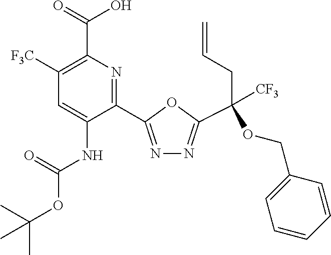 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 71 | 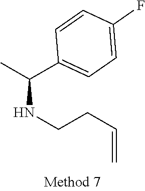 ESI-MS m/z calc. 193.12668, found 194.1 (M + 1)⁺ | 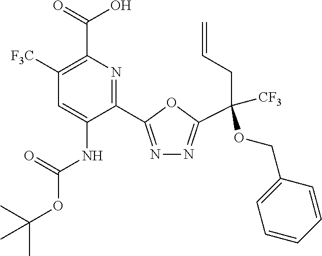 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 72 | 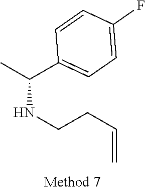 ESI-MS m/z calc. 193.12668, found 194.1 (M + 1)⁺ | 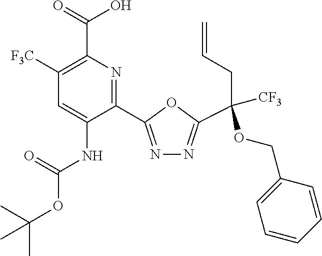 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 73 | 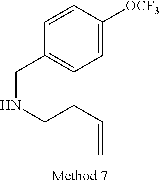 [SMILES: C=CCCNCc1ccc(OC(F)(F)F)cc1] ESI-MS m/z calc. 245.10275, found 246.1 (M + 1)⁺ | 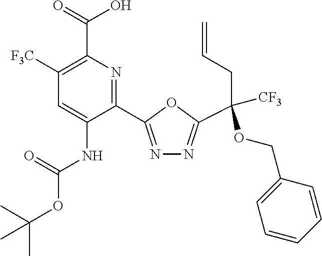 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 74 | 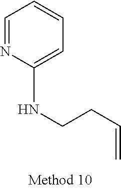 ESI-MS m/z calc. 148.10005, found 149.0 (M + 1)⁺ | 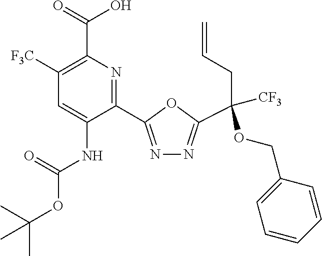 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 75 | 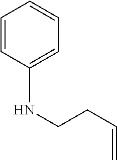 | 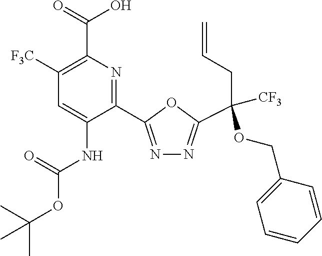 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 76 | 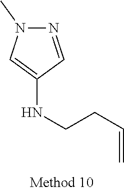 ESI-MS m/z calc. 151.11095, found 152.0 (M + 1)⁺ | 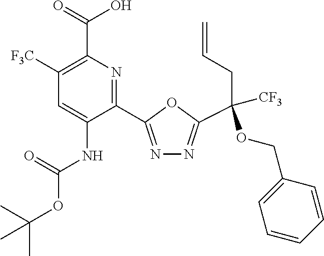 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 77 | 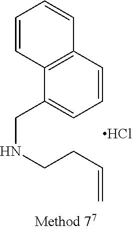 [SMILES: C=CCCNCc1cccc2ccccc12 ; Cl] ESI-MS m/z calc. 211.1361, | 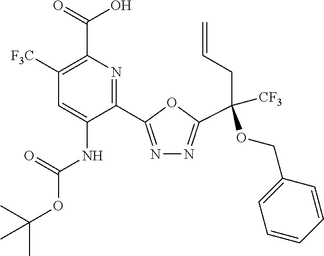 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 212.1 | |
| | (M + 1)⁺ | |
| 78 | 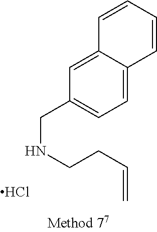 [SMILES: Cl ; C=CCCNCc1ccc2ccccc2c1] ESI-MS m/z calc. 211.1361, | 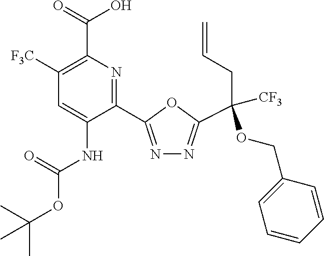 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 212.1 | |
| | (M + 1)⁺ | |
| 79³ | 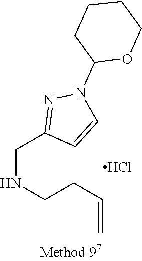 [SMILES: C=CCCNCc1ccn(C2CCCCO2)n1 ; Cl] | 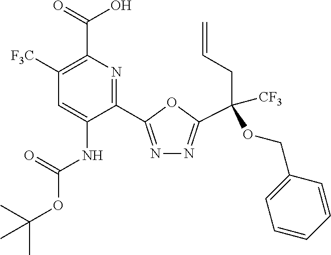 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | ESI-MS m/z | |
| | calc. 235.16846, | |
| | found 236.2 | |
| | (M + 1)⁺ | |
| 80³ | 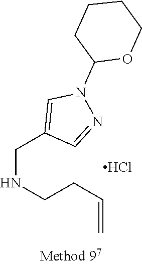 [SMILES: C=CCCNCc1cnn(C2CCCCO2)c1 ; Cl] | 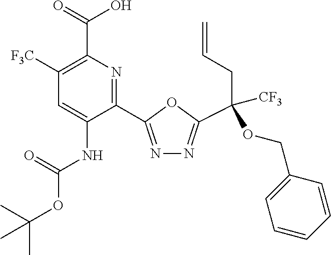 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | ESI-MS m/z | |
| | calc. 235.16846, | |
| | found 236.1 | |
| | (M + 1)⁺ | |
| 81² | 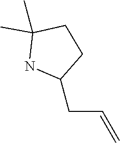 | 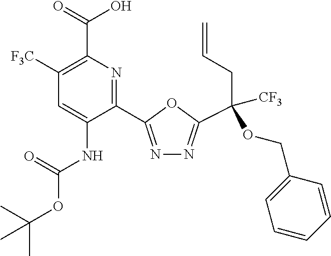 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 82² | 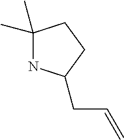 | 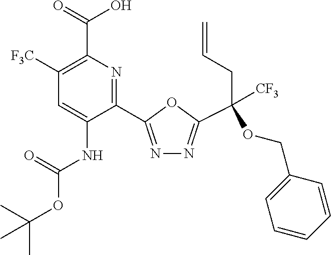 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 83 | 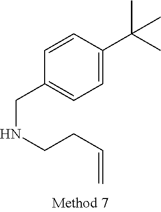 [SMILES: C=CCCNCc1ccc(C(C)(C)C)cc1] ESI-MS m/z calc. 217.18304, | 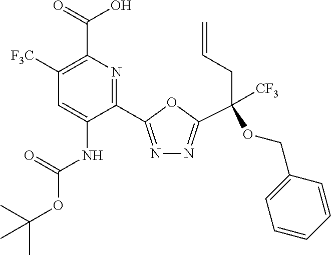 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 218.1 | |
| | (M + 1)⁺ | |
| 84 | 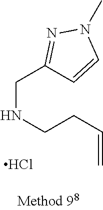 ESI-MS m/z calc. 165.1266, found 166.1 | 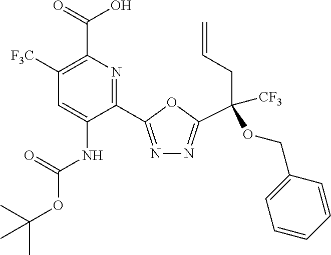 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 85 | 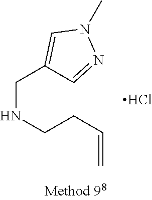 ESI-MS m/z calc. 165.1266, found 166.1 (M + 1)⁺ | 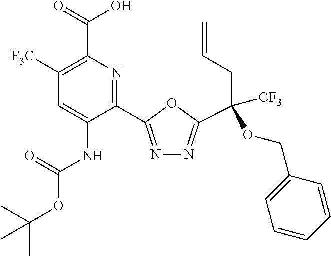 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 86 | 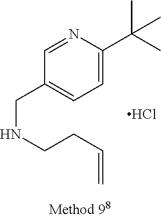 [SMILES: C=CCCNCc1ccc(C(C)(C)C)nc1 ; Cl] ESI-MS m/z calc. 218.1783, found 219.2 (M + 1)⁺ | 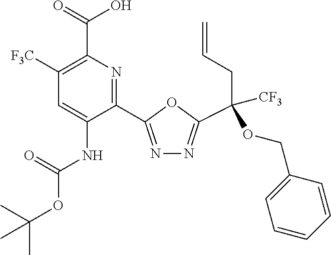 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 87 | 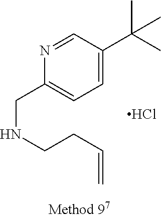 [SMILES: C=CCCNCc1ccc(C(C)(C)C)cn1 ; Cl] ESI-MS m/z calc. 218.1783, | 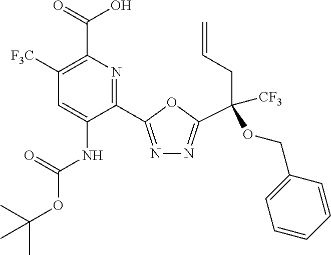 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 219.2 | |
| | (M + 1)⁺ | |
| 88 | 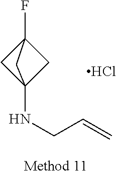 ESI-MS m/z calc. 141.09538, found 142.1 (M + 1)⁺ | 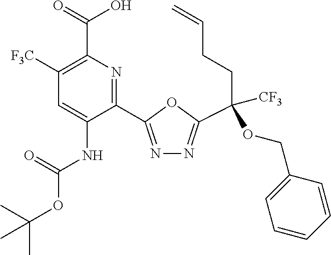 [SMILES: C=CCC[C@@](OCc1ccccc1)(c1nnc(-c2nc(C(=O)O)c(C(F)(F)F)cc2NC(=O)OC(C)(C)C)o1)C(F)(F)F] |
| 89 | 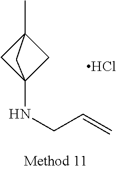 ESI-MS m/z calc. 137.12045, found 138.1 (M + 1)⁺ | 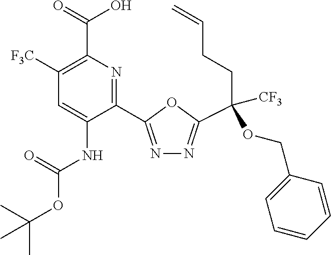 [SMILES: C=CCC[C@@](OCc1ccccc1)(c1nnc(-c2nc(C(=O)O)c(C(F)(F)F)cc2NC(=O)OC(C)(C)C)o1)C(F)(F)F] |
| 90 | 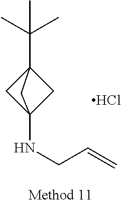 ESI-MS m/z calc. 179.1674, found 180.2 | 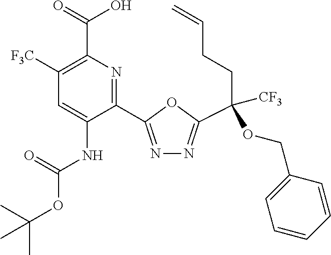 [SMILES: C=CCC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 91 | 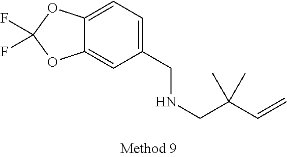 [SMILES: C=CC(C)(C)CNCc1ccc2c(c1)OC(F)(F)O2] ESI-MS m/z calc. 269.12274, found 270.44 (M + 1)⁺ | 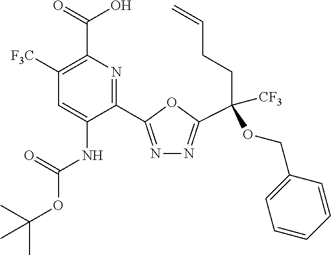 [SMILES: C=CCC[C@@](OCc1ccccc1)(c1nnc(-c2nc(C(=O)O)c(C(F)(F)F)cc2NC(=O)OC(C)(C)C)o1)C(F)(F)F] |
| 92 | 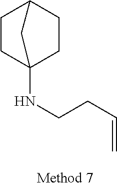 ESI-MS m/z calc. 165.15175, found 166.2 (M + 1)⁺ | 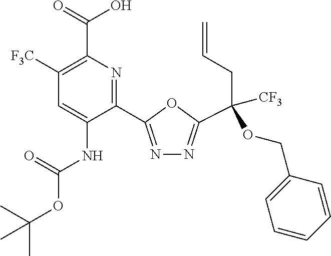 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 93⁴ | 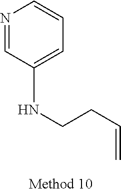 ESI-MS m/z calc. 148.10005, found 149.1 | 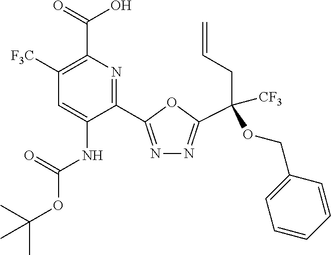 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 94⁴ | 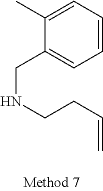 ESI-MS m/z calc. 175.1361, found 176.1 | 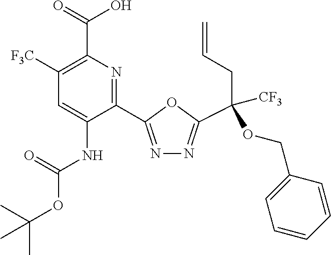 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 95⁴ | 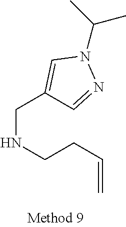 | 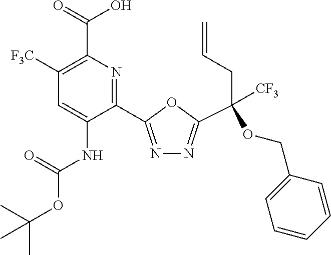 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 96⁴ | 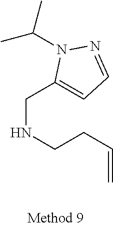 | 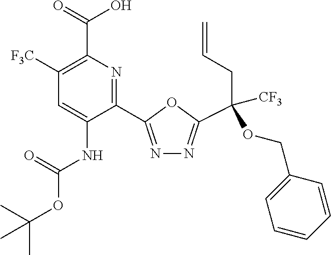 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 97 | 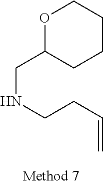 ESI-MS m/z calc. 169.14667, found 170.1 (M + 1)⁺ | 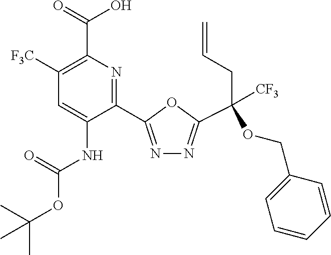 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 98 | 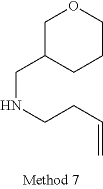 ESI-MS m/z calc. 169.14667, found 170.1 (M + 1)⁺ | 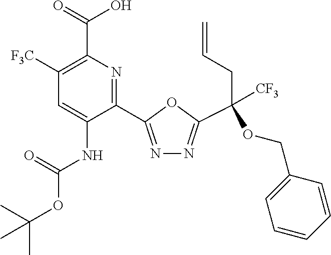 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 99 | 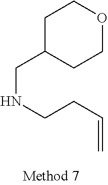 ESI-MS m/z calc. 169.14667, found 170.1 (M + 1)⁺ | 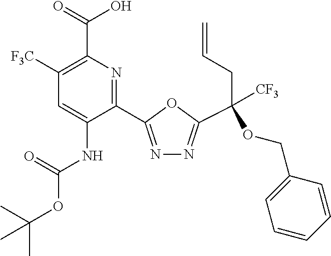 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 100 | 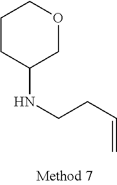 ESI-MS m/z calc. 155.13101, found 156.1 (M + 1)⁺ | 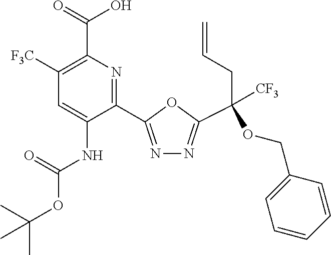 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 101 | 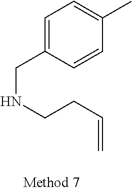 ESI-MS m/z calc. 175.1361, found 176.1 (M + 1)⁺ | 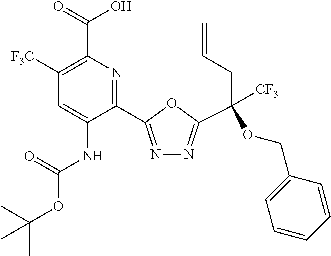 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 102 | 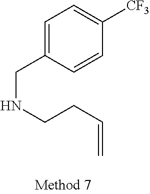 [SMILES: C=CCCNCc1ccc(C(F)(F)F)cc1] ESI-MS m/z calc. 229.10783, found 230.2 (M + 1)⁺ | 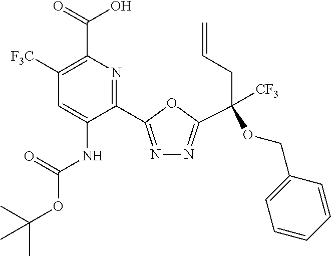 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 103 | 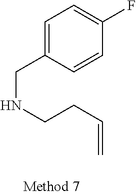 ESI-MS m/z calc. 179.11102, found 180.1 | 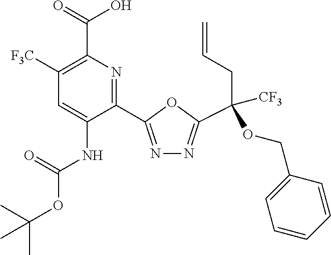 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 104 | 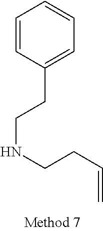 ESI-MS m/z calc. 175.1361, found 176.2 (M + 1)⁺ | 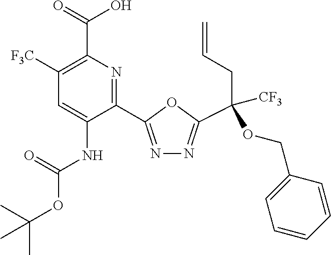 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 105 | 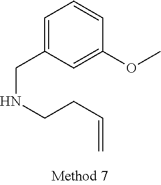 ESI-MS m/z calc. 191.13101, found 192.2 (M + 1)⁺ | 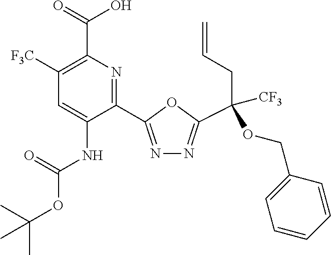 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 106 | 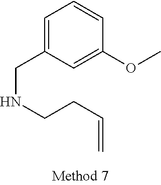 ESI-MS m/z calc. 203.1674, found 204.2 (M + 1)⁺ | 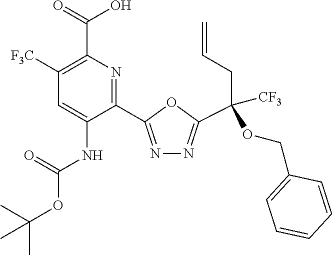 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 107⁴ | 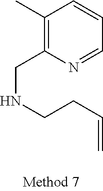 ESI-MS m/z calc. 176.13135, found 177.2 (M + 1)⁺ | 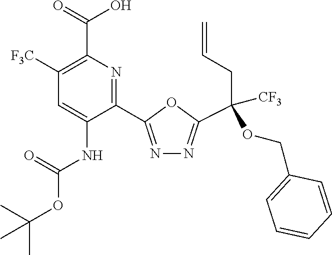 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 108⁴ | 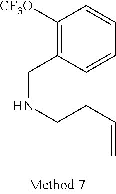 ESI-MS m/z calc. 245.10275, found 246.4 (M + 1)⁺ | 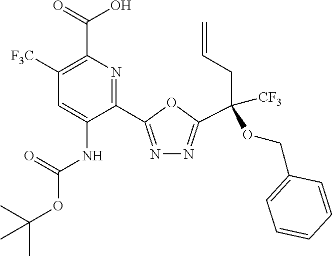 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 109⁴ | 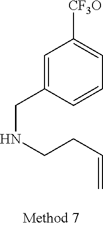 ESI-MS m/z calc. 245.10275, found 246.4 (M + 1)⁺ | 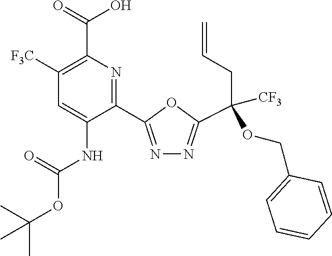 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 110 | 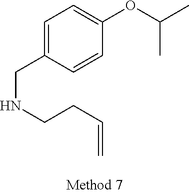 [SMILES: C=CCCNCc1ccc(OC(C)C)cc1] ESI-MS m/z calc. 219.16231, found 220.2 (M + 1)⁺ Retention time: 0.4 minutes. | 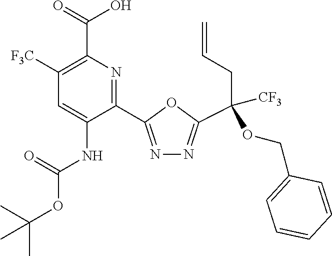 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 111 | 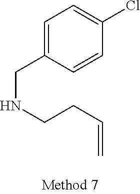 ESI-MS m/z calc. 195.08148, found 196.24 (M + 1)⁺ | 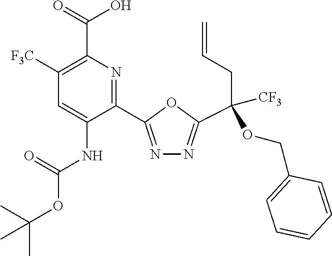 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 112 | 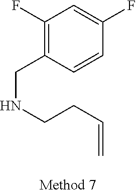 ESI-MS m/z calc. 197.10161, found 198.1 (M + 1)⁺ | 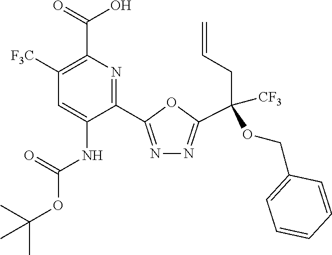 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 113 | 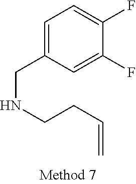 ESI-MS m/z calc. 197.10161, found 198.1 (M + 1)⁺ | 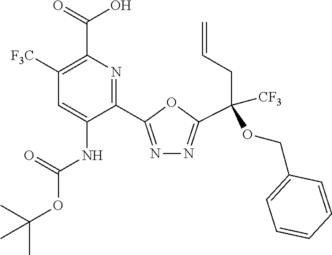 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 114 | 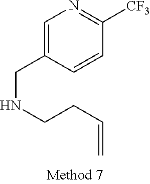 [SMILES: C=CCCNCc1ccc(C(F)(F)F)nc1] ESI-MS m/z calc. 230.10309, found 231.1 (M + 1)⁺ | 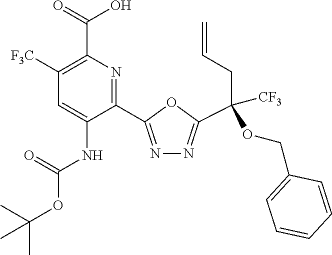 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 115⁵ | 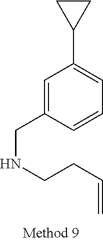 ESI-MS m/z | 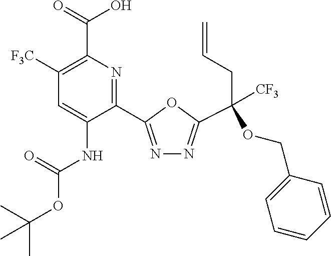 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | calc. 201.15175, | |
| | found 202.12 | |
| | (M + 1)⁺ | |
| 116 | 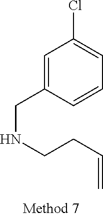 ESI-MS m/z calc. 195.08148, | 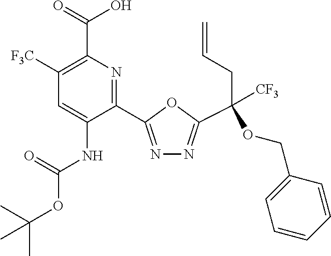 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 196.04 | |
| | (M + 1)⁺ | |
| 117 | 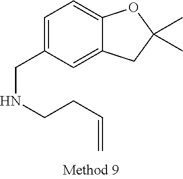 [SMILES: C=CCCNCc1ccc2c(c1)CC(C)(C)O2] ESI-MS m/z calc. 231.16231, found 232.2 (M + 1)⁺ | 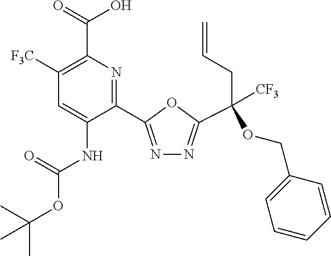 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 118 | 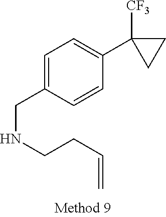 [SMILES: C=CCCNCc1ccc(C2(C(F)(F)F)CC2)cc1] ESI-MS m/z calc. 269.13913, | 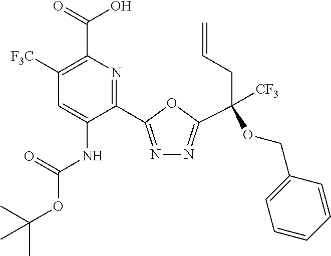 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 270.2 | |
| | (M + 1)⁺ | |
| 119 | 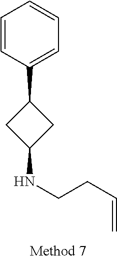 | 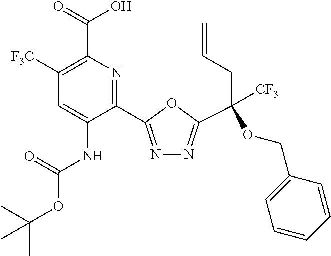 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | ESI-MS m/z | |
| | calc. 201.15175, | |
| | found 202.2 | |
| | (M + 1) | |
| 120⁴ | 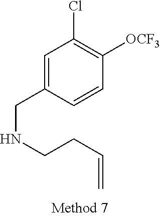 [SMILES: C=CCCNCc1ccc(OC(F)(F)F)c(Cl)c1] ESI-MS m/z calc. 279.06378, | 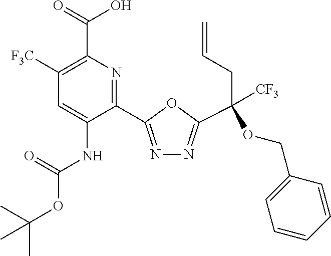 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 280.0 | |
| | (M + 1)⁺ | |
| 121⁴ | 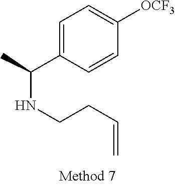 [SMILES: C=CCCN[C@@H](C)c1ccc(OC(F)(F)F)cc1] ESI-MS m/z calc. 259.1184, found 260.1 (M + 1)⁺ | 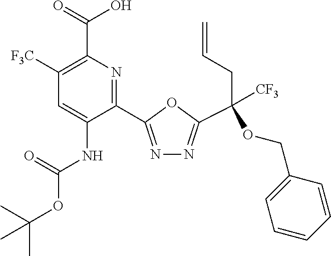 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 122⁴ | 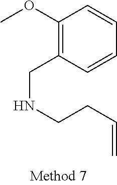 ESI-MS m/z calc. 191.13101, found 192.1 (M + 1)⁺ | 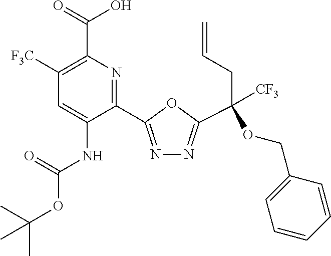 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 123⁴ | 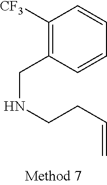 [SMILES: C=CCCNCc1ccccc1C(F)(F)F] ESI-MS m/z calc. 229.10783, found 230.0 (M + 1)⁺ | 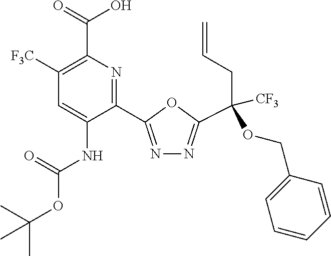 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 124⁴ | 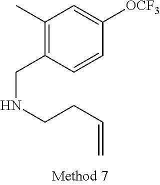 [SMILES: C=CCCNCc1ccc(OC(F)(F)F)cc1C] ESI-MS m/z calc. 259.1184, found 260.1 (M + 1)⁺ | 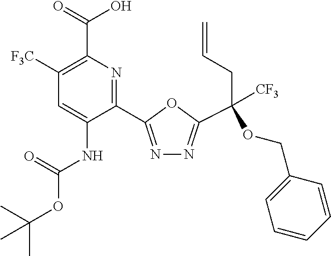 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 125⁴ | 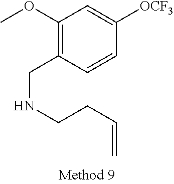 [SMILES: C=CCCNCc1ccc(OC(F)(F)F)cc1OC] ESI-MS m/z calc. 275.1133, found 276.1 (M + 1)⁺ | 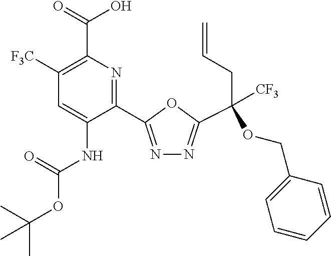 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 126⁴ | 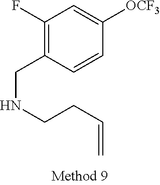 [SMILES: C=CCCNCc1ccc(OC(F)(F)F)cc1F] ESI-MS m/z calc. 263.09332, found 264.4 (M + 1)⁺ | 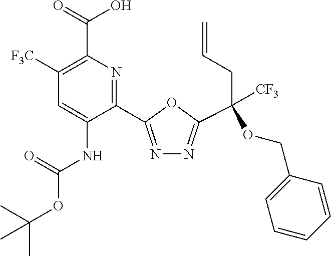 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 127⁴ | 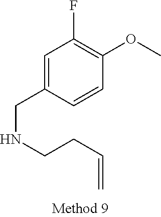 ESI-MS m/z calc. 209.1216, | 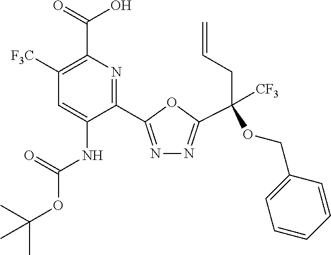 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 211.1 | |
| | (M + 1)⁺ | |
| 128⁴ | 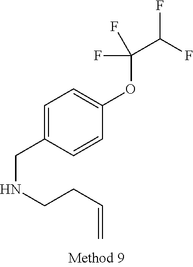 [SMILES: C=CCCNCc1ccc(OC(F)(F)C(F)F)cc1] | 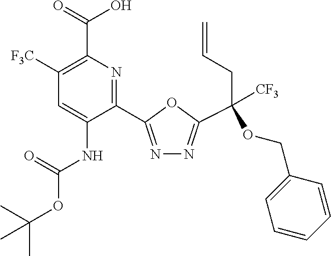 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | ESI-MS m/z | |
| | calc. 277.10898, | |
| | found 278.1 | |
| | (M + 1)⁺ | |
| 129⁵ | 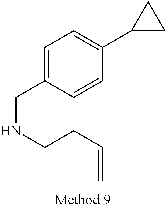 ESI-MS m/z calc. 201.15175, found 202.12 | 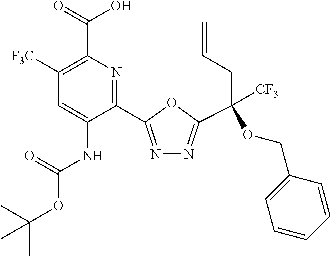 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 130 | 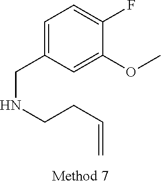 ESI-MS m/z calc. 209.1216, found 210.2 (M + 1)⁺ | 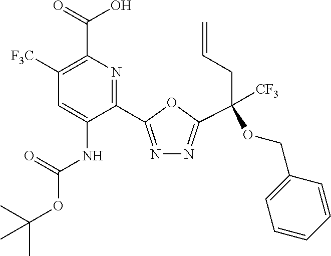 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 131 | 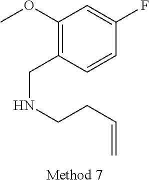 ESI-MS m/z calc. 209.1216, found 210.08 (M + 1)⁺ | 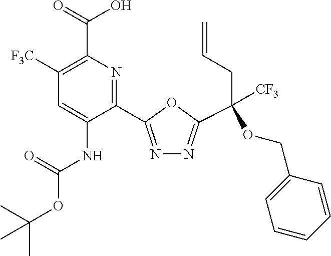 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 132⁴ | 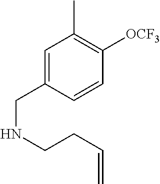 [SMILES: C=CCCNCc1ccc(OC(F)(F)F)c(C)c1] Method 9 ESI-MS m/z calc. 259.1184, found 260.1 (M + 1)⁺ | 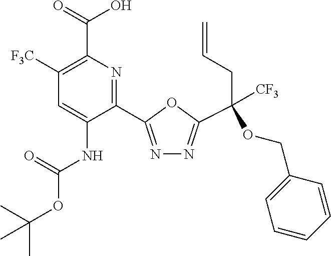 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 133 | 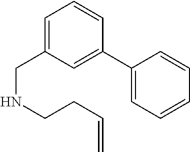 [SMILES: C=CCCNCc1cccc(-c2ccccc2)c1] Method 7 ESI-MS m/z calc. 237.15175, found 238.2 (M + 1)⁺ | 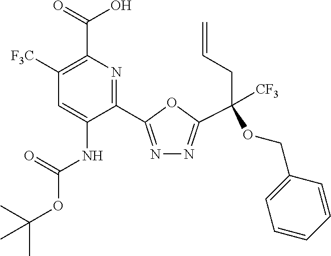 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 134 | 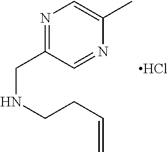 Method 8 ESI-MS m/z calc. 177.1266, found 178.1 (M + 1)⁺ | 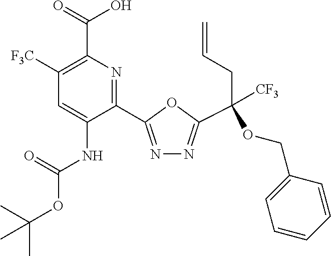 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 135^{4,5} | 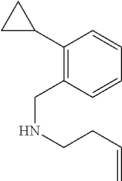 Method 7 ESI-MS m/z calc. 201.15175, found 202.1 | 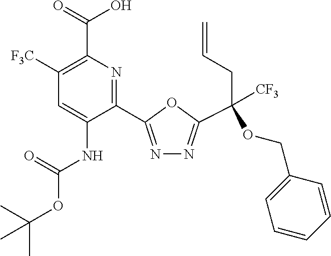 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 136 | 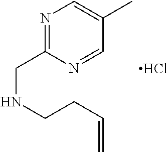 Method 8 ESI-MS m/z calc. 177.1266, found 178.1 (M + 1)⁺ | 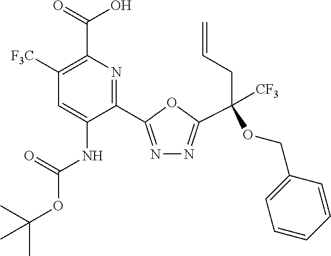 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 137 | 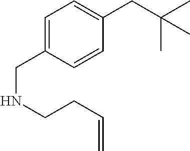 [SMILES: C=CCCNCc1ccc(CC(C)(C)C)cc1] Method 12 ESI-MS m/z calc. 231.1987, found 232.1 (M + 1)⁺ | 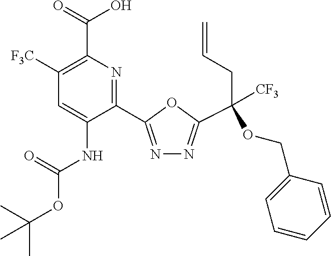 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 138 | 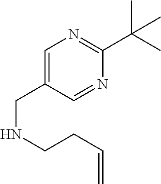 [SMILES: C=CCCNCc1cnc(C(C)(C)C)nc1] Method 9 ESI-MS m/z calc. 219.17355, found 220.1 | 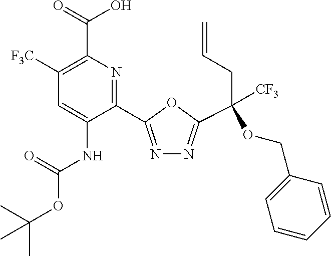 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 139 | 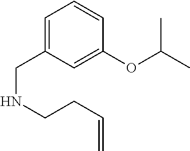 [SMILES: C=CCCNCc1cccc(OC(C)C)c1] Method 9 ESI-MS m/z calc. 219.16231, found 220.2 (M + 1)⁺ | 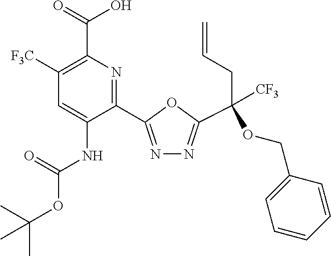 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 140 | 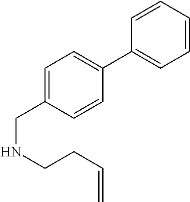 [SMILES: C=CCCNCc1ccc(-c2ccccc2)cc1] Method 7 ESI-MS m/z calc. 237.15175, | 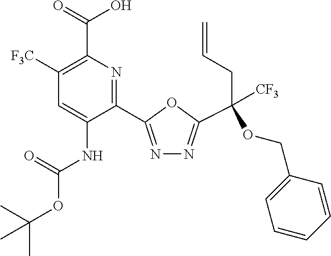 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 238.1 | |
| | (M + 1)⁺ | |
| 141 | 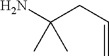 | 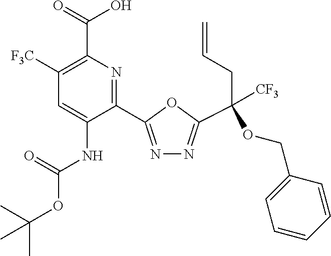 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 142 | 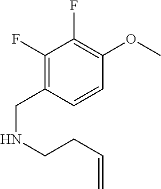 [SMILES: C=CCCNCc1ccc(OC)c(F)c1F] Method 9 ESI-MS m/z calc. 227.11217, found 228.052 | 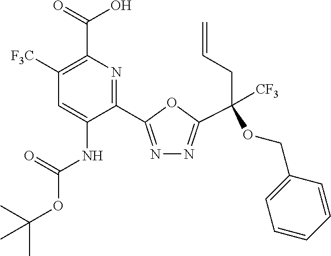 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 143 | 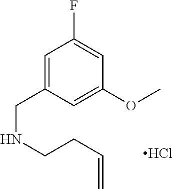 Method 8 ESI-MS m/z calc. 209.1216, found 210.08 (M + 1)⁺ | 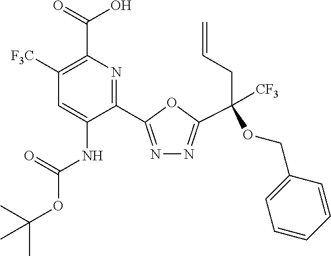 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 144 | 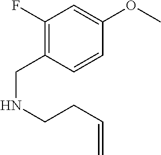 Method 9 ESI-MS m/z calc. 209.1216, found 210.05 (M + 1)⁺ | 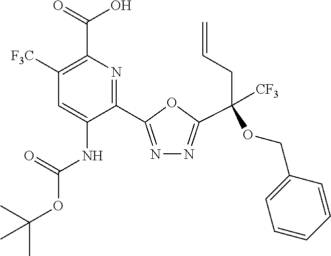 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 145⁴ | 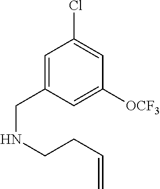 [SMILES: C=CCCNCc1cc(Cl)cc(OC(F)(F)F)c1] Method 9 ESI-MS m/z calc. 279.06378, found 280.0 (M + 1)⁺ | 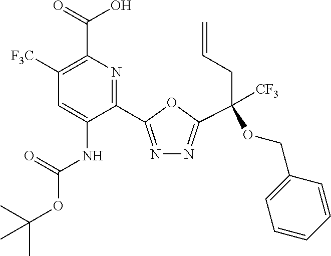 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 146⁴ | 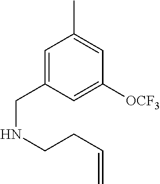 [SMILES: C=CCCNCc1cc(C)cc(OC(F)(F)F)c1] Method 9 ESI-MS m/z calc. 259.1184, found 260.1 (M + 1)⁺ | 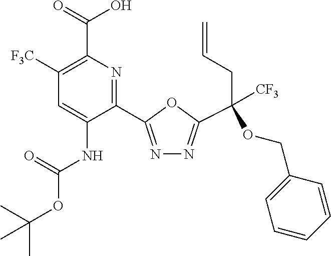 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 147 | 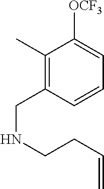 [SMILES: C=CCCNCc1cccc(OC(F)(F)F)c1C] Method 9 ESI-MS m/z calc. 259.1184, found 260.1 (M + 1)⁺ | 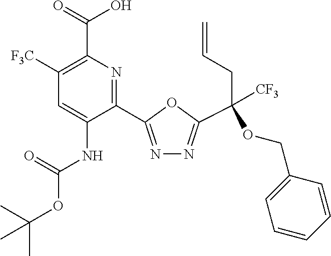 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 148 | 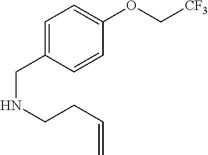 [SMILES: C=CCCNCc1ccc(OCC(F)(F)F)cc1] Method 9 ESI-MS m/z calc. 259.1184, found 260.1 | 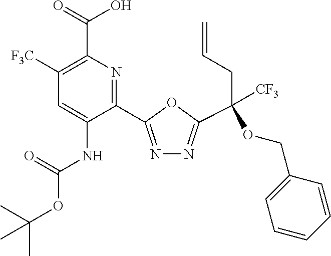 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 149 | 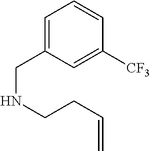 [SMILES: C=CCCNCc1cccc(C(F)(F)F)c1] Method 7 ESI-MS m/z calc. 229.10783, found 230.2 (M + 1)⁺ | 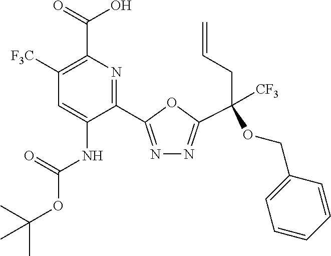 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 150⁶ | 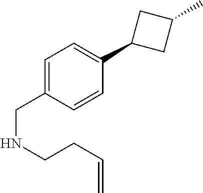 [SMILES: C=CCCNCc1ccc([C@H]2C[C@H](C)C2)cc1] Method 12 ESI-MS m/z calc. 227.1674, found 228.12 | 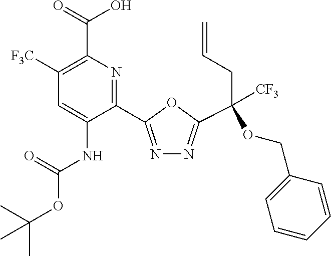 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | (M + 1)⁺ | |
| 151 | 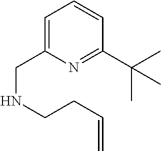 [SMILES: C=CCCNCc1cccc(C(C)(C)C)n1] Method 9 ESI-MS m/z calc. 218.1783, found 219.1 (M + 1)⁺ | 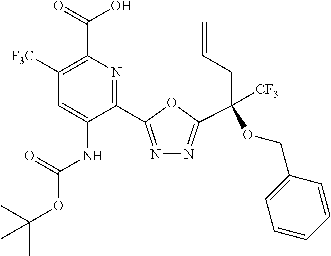 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 152⁴ | 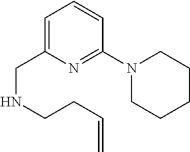 [SMILES: C=CCCNCc1cccc(N2CCCCC2)n1] Method 9 ESI-MS m/z calc. 245.1892, found 246.3 (M + 1)⁺ | 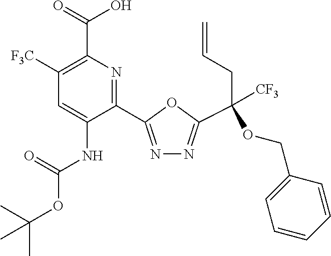 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 153 | 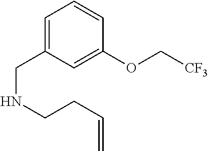 [SMILES: C=CCCNCc1cccc(OCC(F)(F)F)c1] Method 9 ESI-MS m/z calc. 259.1184, found 260.1 (M + 1)⁺ | 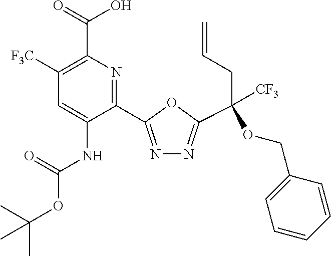 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| 154 | 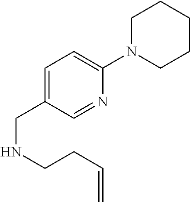 [SMILES: C=CCCNCc1ccc(N2CCCCC2)nc1] Method 9 ESI-MS m/z calc. 245.1892, | 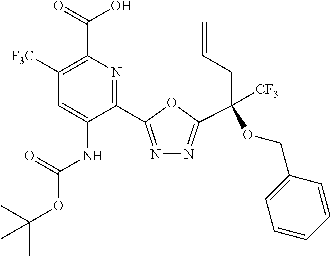 [SMILES: C=CC[C@@](OCc1ccccc1)(c1nnc(-c2nc(C(=O)O)c(C(F)(F)F)cc2NC(=O)OC(C)(C)C)o1)C(F)(F)F] |
| | found 246.2 | |
| | (M + 1)⁺ | |

Final Examples Prepared by Method 13 and Analogous to the
Representative Procedure Described for Example 40

| Example | | Final Product |
|---|---|---|
| number | Final Product structure | IUPAC Name |
| 41 | 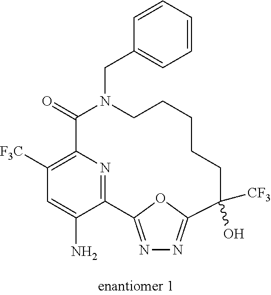 [SMILES: Nc1cc(C(F)(F)F)c2nc1-c1nnc(o1)C(O)(C(F)(F)F)CCCCCN(Cc1ccccc1)C2=O] | 17-Amino-12- benzyl-6- hydroxy-6,15- bis (trifluoromethyl)- 19-oxa- 3,4,12,18- tetrazatricyclo [12.3.1.12,5] nonadeca- 1(18),2,4,14,1 6-pentaen-13- one (enantiomer 1) (Compound 74) |
| 42 | 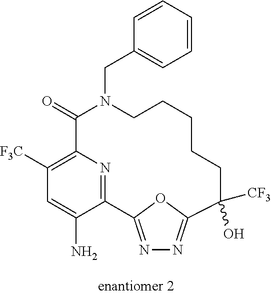 [SMILES: Nc1cc(C(F)(F)F)c2nc1-c1nnc(o1)C(O)(C(F)(F)F)CCCCCN(Cc1ccccc1)C2=O] | 17-Amino-12- benzyl-6- hydroxy-6,15-bis (trifluoromethyl)- 19-oxa-3,4,12, 18-tetrazatricyclo [12.3.1.12,5] nonadeca- 1(18),2,4,14,1 6-pentaen-13- one (enantiomer 2) (Compound 75) |
| 43 | 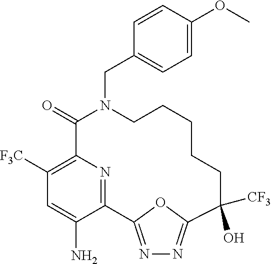 [SMILES: COc1ccc(CN2CCCCC[C@](O)(C(F)(F)F)c3nnc(o3)-c3nc(c(C(F)(F)F)cc3N)C2=O)cc1] | (6R)-17- Amino-6- hydroxy-12-[(4- methoxyphenyl) methyl]-6,15-bis (trifluoromethyl)- 19-oxa- 3,4,12,18- tetrazatricyclo [12.3.1.12,5] nonadeca- 1(18),2,4,14,16- pentaen-13-one (Compound 76) |
| 44 | 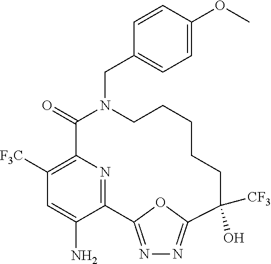 [SMILES: COc1ccc(CN2CCCCC[C@@](O)(C(F)(F)F)c3nnc(o3)-c3nc(c(C(F)(F)F)cc3N)C2=O)cc1] | (6S)-17- Amino-6- hydroxy-12-[(4- methoxyphenyl) methyl]- 6,15-bis (trifluoromethyl)- 19-oxa- 3,4,12,18- tetrazatricyclo [12.3.1.12,5] nonadeca- 1(18),2,4,14,1 6-pentaen-13- one (Compound 77) |
| 45 | 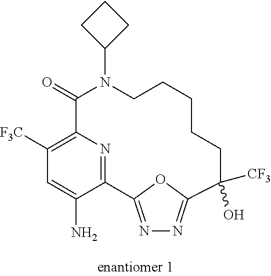 [SMILES: Nc1cc(C(F)(F)F)c2nc1-c1nnc(o1)C(O)(C(F)(F)F)CCCCCN(C1CCC1)C2=O] | 17-Amino-12- cyclobutyl-6- hydroxy-6,15-bis (trifluoromethyl)- 19-oxa- 3,4,12,18- tetrazatricyclo [12.3.1.12,5] nonadeca- 1(18),2,4,14,1 6-pentaen-13-one (enantiomer 1) (Compound 78) |
| 46 | 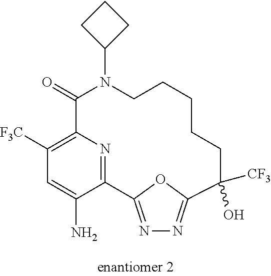 [SMILES: Nc1cc(C(F)(F)F)c2nc1-c1nnc(o1)C(O)(C(F)(F)F)CCCCCN(C1CCC1)C2=O] | 17-Amino-12- cyclobutyl-6- hydroxy-6,15- bis (trifluoromethyl)- 19-oxa-3,4,12,18- tetrazatricyclo [12.3.1.12,5] nonadeca- 1(18),2,4,14,1 6-pentaen-13-one (enantiomer 2) (Compound 79) |
| 47 | 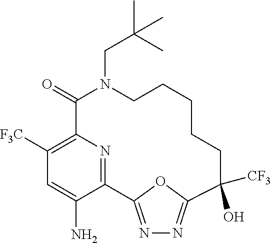 [SMILES: CC(C)(C)CN1CCCCC[C@](O)(C(F)(F)F)c2nnc(o2)-c2nc(c(C(F)(F)F)cc2N)C1=O] | (6R)-17- Amino-12-(2,2- dimethylpropyl)- 6-hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 80) |
| 48 | 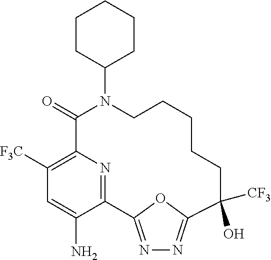 [SMILES: Nc1cc(C(F)(F)F)c2nc1-c1nnc(o1)[C@@](O)(C(F)(F)F)CCCCCN(C1CCCCC1)C2=O] | (6R)-17-Amino-12- cyclohexyl-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 81) |
| 49 | 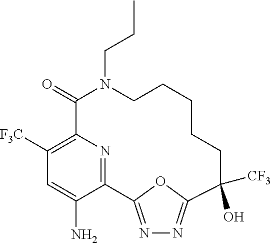 [SMILES: CCCN1CCCCC[C@](O)(C(F)(F)F)c2nnc(o2)-c2nc(c(C(F)(F)F)cc2N)C1=O] | (6R)-17-Amino-6- hydroxy-12- propyl-6,15-bis (trifluoromethyl)- 19-oxa-3,4,12,18- tetrazatricyclo [12.3.1.12,5] nonadeca- 1(18),2,4,14,1 6-pentaen-13-one (Compound 82) |
| 50 | 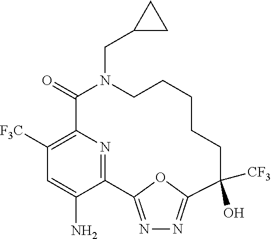 [SMILES: Nc1cc(C(F)(F)F)c2nc1-c1nnc(o1)[C@@](O)(C(F)(F)F)CCCCCN(CC1CC1)C2=O] | (6R)-17- Amino-12- (cyclopropylmethyl)- 6-hydroxy-6,15- bis(trifluoromethyl)- 19-oxa- 3,4,12,18- tetrazatricyclo [12.3.1.12,5] nonadeca- 1(18),2,4,14,1 6-pentaen-13-one (Compound 83) |
| 51 | 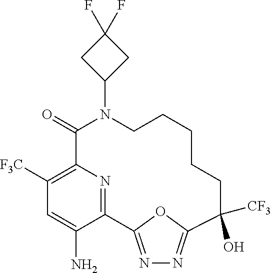 [SMILES: Nc1cc(C(F)(F)F)c2nc1-c1nnc(o1)[C@@](O)(C(F)(F)F)CCCCCN(C1CC(F)(F)C1)C2=O] | (6R)-17- Amino-12-(3,3- difluorocyclobutyl)- 6-hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 84) |
| 52 | 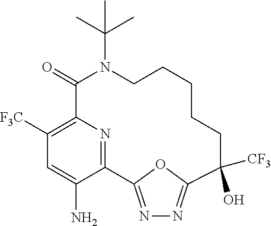 [SMILES: CC(C)(C)N1CCCCC[C@](O)(C(F)(F)F)c2nnc(o2)-c2nc(c(C(F)(F)F)cc2N)C1=O] | (6R)-17- Amino-12- tert-butyl-6- hydroxy-6,15- bis(trifluorom ethyl)-19-oxa- 3,4,12,18- tetrazatricyclo [12.3.1.12,5] nonadeca- 1(18),2,4,14,1 6-pentaen-13-one (Compound 85) |
| 53 | 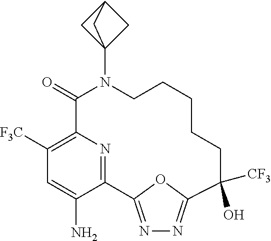 [SMILES: Nc1cc(C(F)(F)F)c2nc1-c1nnc(o1)[C@@](O)(C(F)(F)F)CCCCCN(C13CC(C1)C3)C2=O] | (6R)-17- Amino-12-(1- bicyclo[1.1.1] pentanyl)-6- hydroxy-6,15- bis (trifluoromethyl)- 19-oxa-3,4,12,18- tetrazatricyclo [12.3.1.12,5] nonadeca- 1(18),2,4,14,1 6-pentaen-13-one (Compound 86) |
| 54 | 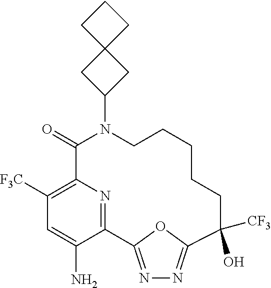 [SMILES: Nc1cc(C(F)(F)F)c2nc1-c1nnc(o1)[C@@](O)(C(F)(F)F)CCCCCN(C1CC3(CCC3)C1)C2=O] | (6R)-17- Amino-6- hydroxy-12- spiro[3.3]heptan- 2-yl-6,15- bis(trifluoromethyl)- 19-oxa-3,4,12,18- tetrazatricyclo [12.3.1.12,5] nonadeca- 1(18),2,4,14,1 6-pentaen-13-one (Compound 87) |
| 55 | 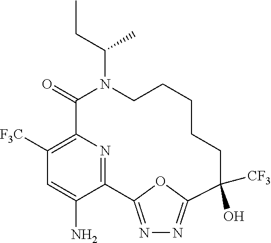 [SMILES: CC[C@H](C)N1CCCCC[C@](O)(C(F)(F)F)c2nnc(o2)-c2nc(c(C(F)(F)F)cc2N)C1=O] | (6R)-17-Amino-6- hydroxy-12-[(1S)-1- methylpropyl]- 6,15-bis (trifluoromethyl)- 19-oxa-3,4,12,18- tetrazatricyclo [12.3.1.12,5] nonadeca- 1(18),2,4,14,1 6-pentaen-13-one (Compound 88) |
| 56 | 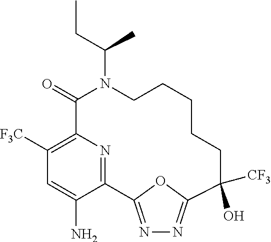 [SMILES: CC[C@@H](C)N1CCCCC[C@](O)(C(F)(F)F)c2nnc(o2)-c2nc(c(C(F)(F)F)cc2N)C1=O] | (6R)-17-Amino-6- hydroxy-12- [(1R)-1- methylpropyl]- 6,15-bis (trifluoromethyl)- 19-oxa-3,4,12,18- tetrazatricyclo [12.3.1.12,5] nonadeca- 1(18),2,4,14,1 6-pentaen-13-one (Compound 89) ( |
| 57 | 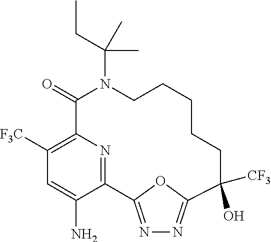 [SMILES: CCC(C)(C)N1CCCCC[C@](O)(C(F)(F)F)c2nnc(o2)-c2nc(c(C(F)(F)F)cc2N)C1=O] | (6R)-17- Amino-12-(1,1- dimethylpropyl)- 6-hydroxy-6,15- bis(trifluoromethyl)- 19-oxa-3,4,12,18- tetrazatricyclo [12.3.1.12,5] nonadeca- 1(18),2,4,14,1 6-pentaen-13-one (Compound 90) |
| 58 | 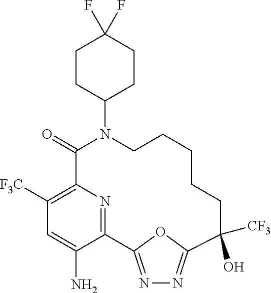 [SMILES: Nc1cc(C(F)(F)F)c2nc1-c1nnc(o1)[C@@](O)(C(F)(F)F)CCCCCN(C1CCC(F)(F)CC1)C2=O] | (6R)-17- Amino-12- (4,4- difluorocyclohexyl)-6- hydroxy-6,15-bis (trifluoromethyl)- 19-oxa- 3,4,12,18- tetrazatricyclo [12.3.1.12,5] nonadeca- 1(18),2,4,14,1 6-pentaen-13- one (Compound 91) |
| 59 | 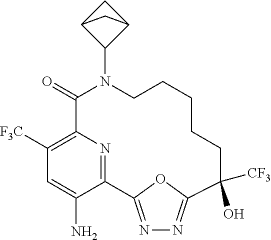 [SMILES: Nc1cc(C(F)(F)F)c2nc1-c1nnc(o1)[C@@](O)(C(F)(F)F)CCCCCN(C1C3CC1C3)C2=O] | (6R)-17- Amino-12-(2- bicyclo[1.1.1] pentanyl)-6- hydroxy-6,15-bis( trifluoromethyl)- 19-oxa-3,4,12,18- tetrazatricyclo [12.3.1.12,5] nonadeca- 1(18),2,4,14,1 6-pentaen-13-one (Compound 92) |
| 60 | 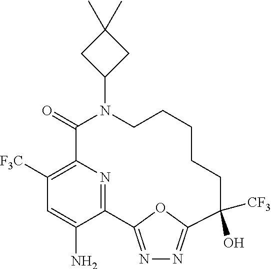 [SMILES: CC1(C)CC(N2CCCCC[C@](O)(C(F)(F)F)c3nnc(o3)-c3nc(c(C(F)(F)F)cc3N)C2=O)C1] | (6R)-17- Amino-12- (3,3- dimethylcyclobutyl)- 6-hydroxy-6,15- bis (trifluoromethyl)- 19-oxa- 3,4,12,18- tetrazatricyclo [12.3.1.12,5] nonadeca- 1(18),2,4,14,1 6-pentaen-13- one (Compound 93) |
| 61 | 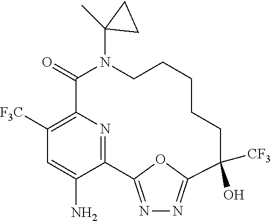 [SMILES: CC1(N2CCCCC[C@](O)(C(F)(F)F)c3nnc(o3)-c3nc(c(C(F)(F)F)cc3N)C2=O)CC1] | (6R)-17- Amino-6- hydroxy-12-(1- methylcyclopropyl)- 6,15-bis (trifluoromethyl)- 19-oxa- 3,4,12,18- tetrazatricyclo [12.3.1.12,5] nonadeca- 1(18),2,4,14,1 6-pentaen-13-one (Compound94) |
| 62 | 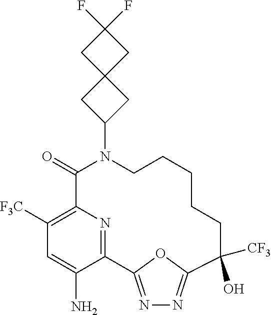 [SMILES: Nc1cc(C(F)(F)F)c2nc1-c1nnc(o1)[C@@](O)(C(F)(F)F)CCCCCN(C1CC3(C1)CC(F)(F)C3)C2=O] | (6R)-17- Amino-12-(2,2- difluorospiro [3.3]heptan-6- yl)-6-hydroxy-6,15- bis (trifluoromethyl)- 19-oxa-3,4,12,18- tetrazatricyclo [12.3.1.12,5] nonadeca- 1(18),2,4,14,1 6-pentaen-13- one (Compound 95) |
| 63 | 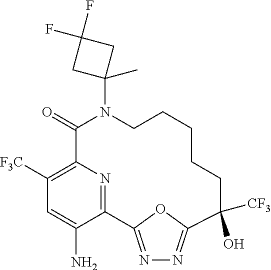 [SMILES: CC1(N2CCCCC[C@](O)(C(F)(F)F)c3nnc(o3)-c3nc(c(C(F)(F)F)cc3N)C2=O)CC(F)(F)C1] | (6R)-17- Amino-12- (3,3-difluoro- 1-methyl- cyclobutyl)-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 96) |
| 64 | 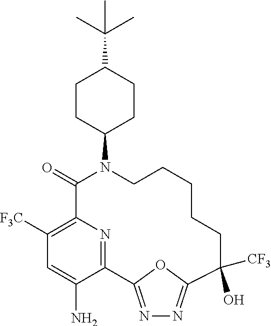 | (6R)-17- Amino-12- (trans-4-tert- butylcyclohexyl)- 6-hydroxy- 6,15-bis (trifluoromethyl)- 19-oxa- 3,4,12,18- tetrazatricyclo [12.3.1.12,5] nonadeca- 1(18),2,4,14,1 6-pentaen-13- one (Compound 97) |
| 65 | 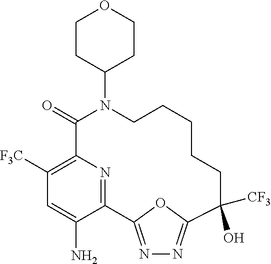 [SMILES: Nc1cc(C(F)(F)F)c2nc1-c1nnc(o1)[C@@](O)(C(F)(F)F)CCCCCN(C1CCOCC1)C2=O] | (6R)-17- Amino-6- hydroxy-12- tetrahydropyran- 4-yl-6,15- bis (trifluoromethyl)- 19-oxa- 3,4,12,18- tetrazatricyclo [12.3.1.12,5] nonadeca- 1(18),2,4,14,1 6-pentaen-13-one (Compound 98) |
| 66¹ | 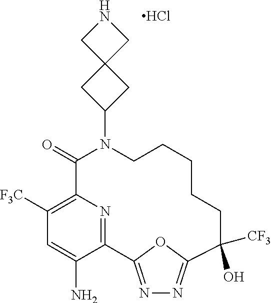 [SMILES: Cl ; Nc1cc(C(F)(F)F)c2nc1-c1nnc(o1)[C@@](O)(C(F)(F)F)CCCCCN(C1CC3(CNC3)C1)C2=O] | (6R)-17- Amino-12-(2- azaspiro[3.3] heptan-6-yl)-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 99) |
| 67 | 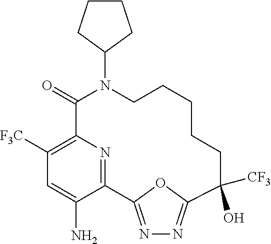 [SMILES: Nc1cc(C(F)(F)F)c2nc1-c1nnc(o1)[C@@](O)(C(F)(F)F)CCCCCN(C1CCCC1)C2=O] | (6R)-17- Amino-12- cyclopentyl-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 100) |
| 68 | 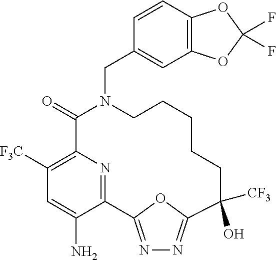 [SMILES: Nc1cc(C(F)(F)F)c2nc1-c1nnc(o1)[C@@](O)(C(F)(F)F)CCCCCN(Cc1ccc3c(c1)OC(F)(F)O3)C2=O] | (6R)-Amino- 12-[(2,2- difluoro-1,3- benzodioxol- 5-yl)methyl]- 6-hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 101) |
| 69 | 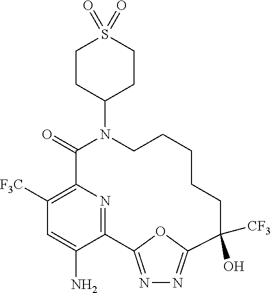 [SMILES: Nc1cc(C(F)(F)F)c2nc1-c1nnc(o1)[C@@](O)(C(F)(F)F)CCCCCN(C1CCS(=O)(=O)CC1)C2=O] | (6R)-17- Amino-12- (1,1- dioxothian-4- yl)-6-hydroxy- 6,15- bis (trifluoromethyl)- 19-oxa- 3,4,12,18- tetrazatricyclo [12.3.1.12,5] nonadeca- 1(18),2,4,14,1 6-pentaen-13- one (Compound 102) |
| 70 | 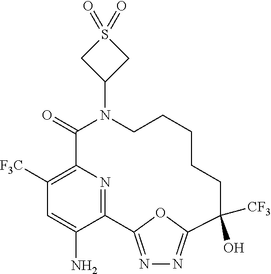 [SMILES: Nc1cc(C(F)(F)F)c2nc1-c1nnc(o1)[C@@](O)(C(F)(F)F)CCCCCN(C1CS(=O)(=O)C1)C2=O] | (6R)-17- Amino-12-(1,1- dioxothietan- 3-yl)-6- hydroxy-6,15- bis (trifluoromethyl)- 19-oxa-3,4,12,18- tetrazatricyclo [12.3.1.12,5] nonadeca- 1(18),2,4,14,1 6-pentaen-13- one (Compound 103) |
| 71 | 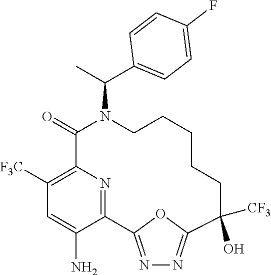 [SMILES: C[C@@H](c1ccc(F)cc1)N1CCCCC[C@](O)(C(F)(F)F)c2nnc(o2)-c2nc(c(C(F)(F)F)cc2N)C1=O] | (6R)-17- Amino-12- [(1S)-1-(4- fluorophenyl) ethyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 104) |
| 72 | 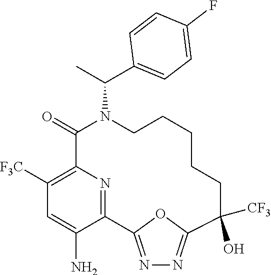 [SMILES: C[C@H](c1ccc(F)cc1)N1CCCCC[C@](O)(C(F)(F)F)c2nnc(o2)-c2nc(c(C(F)(F)F)cc2N)C1=O] | (6R)-17-Amino-12- [(1R)-1-(4- fluorophenyl) ethyl]-6- hydroxy-6,15-bis (trifluoromethyl)- 19-oxa- 3,4,12,18- tetrazatricyclo [12.3.1.12,5] nonadeca- 1(18),2,4,14,1 6-pentaen-13-one (Compound 105) |
| 73 | 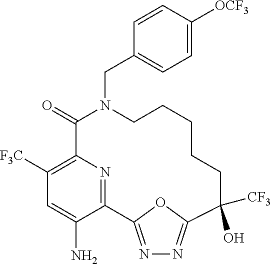 [SMILES: Nc1cc(C(F)(F)F)c2nc1-c1nnc(o1)[C@@](O)(C(F)(F)F)CCCCCN(Cc1ccc(OC(F)(F)F)cc1)C2=O] | (6R)-17-Amino-6- hydroxy-12-[[4- (trifluoromethoxy) phenyl] methyl]-6,15-bis (trifluorom 19-oxa-ethyl)- 3,4,12,18- tetrazatricyclo [12.3.1.12,5] nonadeca- 1(18),2,4,14,1 6-pentaen-13-one (Compound 106) |
| 74 | 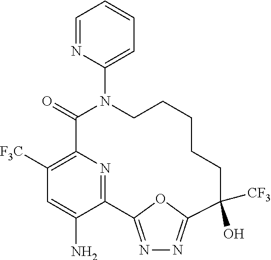 [SMILES: Nc1cc(C(F)(F)F)c2nc1-c1nnc(o1)[C@@](O)(C(F)(F)F)CCCCCN(c1ccccn1)C2=O] | (6R)-17-Amino-6- hydroxy-12- (2-pyridyl)- 6,15-bis (trifluoromethyl)- 19-oxa- 3,4,12,18- tetrazatricyclo [12.3.1.12,5] nonadeca- 1(18),2,4,14,1 6-pentaen-13-one (Compound 107) |
| 75 | 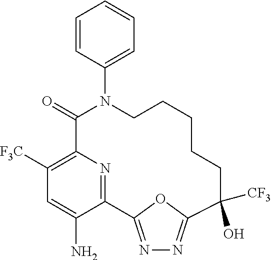 [SMILES: Nc1cc(C(F)(F)F)c2nc1-c1nnc(o1)[C@@](O)(C(F)(F)F)CCCCCN(c1ccccc1)C2=O] | (6R)-17-Amino-6- hydroxy-12- phenyl-6,15-bis (trifluorom 19-oxa-ethyl)- 3,4,12,18- tetrazatricyclo [12.3.1.12,5] nonadeca- 1(18),2,4,14,1 6-pentaen-13-one (Compound 108) |
| 76 | 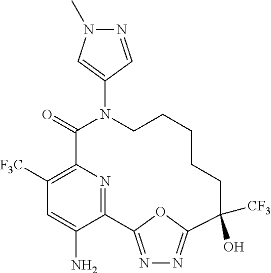 [SMILES: Cn1cc(N2CCCCC[C@](O)(C(F)(F)F)c3nnc(o3)-c3nc(c(C(F)(F)F)cc3N)C2=O)cn1] | (6R)-17-Amino-6- hydroxy-12-(1- methylpyrazol- 4-yl)-6,15-bis (trifluoromethyl)- 19-oxa-3,4,12,18- tetrazatricyclo [12.3.1.12,5] nonadeca- 1(18),2,4,14,1 6-pentaen-13-one (Compound 109) |
| 77 | 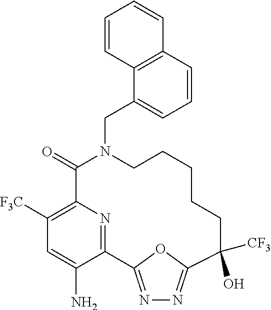 [SMILES: Nc1cc(C(F)(F)F)c2nc1-c1nnc(o1)[C@@](O)(C(F)(F)F)CCCCCN(Cc1cccc3ccccc13)C2=O] | (6R)-17- Amino-6- hydroxy-12-(1- naphthylmethyl)- 6,15-bis (trifluoromethyl)- 19-oxa-3,4,12,18- tetrazatricyclo [12.3.1.12,5] nonadeca- 1(18),2,4,14,1 6-pentaen-13-one (Compound 110) |
| 78 | 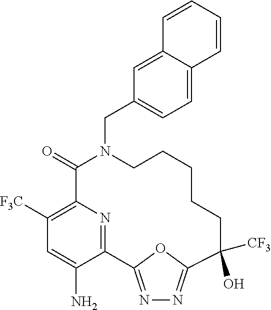 [SMILES: Nc1cc(C(F)(F)F)c2nc1-c1nnc(o1)[C@@](O)(C(F)(F)F)CCCCCN(Cc1ccc3ccccc3c1)C2=O] | (6R)-17-Amino-6- hydroxy-12-(2- naphthylmethyl)- 6,15-bis (trifluoromethyl)- 19-oxa-3,4,12,18- tetrazatricyclo [12.3.1.12,5] nonadeca- 1(18),2,4,14,1 6-pentaen-13-one (Compound 111) |
| 79³ | 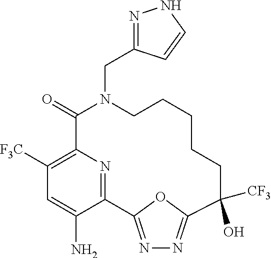 [SMILES: Nc1cc(C(F)(F)F)c2nc1-c1nnc(o1)[C@@](O)(C(F)(F)F)CCCCCN(Cc1cc[nH]n1)C2=O] | (6R)-17-Amino-6- hydroxy-12- (1H-pyrazol- 3-ylmethyl)-6,15-bis (trifluoromethyl)- 19-oxa-3,4,12,18- tetrazatricyclo [12.3.1.12,5] nonadeca- 1(18),2,4,14,1 6-pentaen-13-one (Compound 112) |
| 80³ | 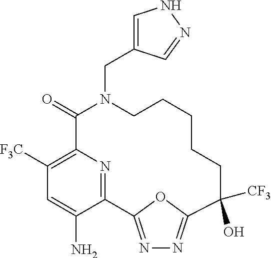 [SMILES: Nc1cc(C(F)(F)F)c2nc1-c1nnc(o1)[C@@](O)(C(F)(F)F)CCCCCN(Cc1cn[nH]c1)C2=O] | (6R)-17- Amino-6- hydroxy-12- (1H-pyrazol- 4-ylmethyl)- 6,15-bis (trifluoromethyl)- 19-oxa-3,4,12,18- tetrazatricyclo [12.3.1.12,5] nonadeca- 1(18),2,4,14,1 6-pentaen-13-one (Compound 113) |
| 81² | 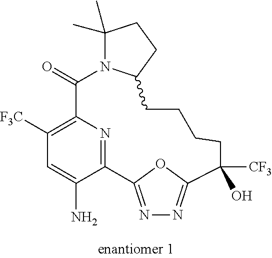 [SMILES: CC1(C)CCC2CCCC[C@](O)(C(F)(F)F)c3nnc(o3)-c3nc(c(C(F)(F)F)cc3N)C(=O)N21] | (6R)-20- Amino-6- hydroxy-14,14- dimethyl-6,18-bis (trifluoromethyl)- 22-oxa- 3,4,15,21- tetraazatetracy clo[15.3.1.12, 5.011,15]docosa- 1(21),2,4,17,1 9-pentaen-16-one (enantiomer 1) (Compound 114) |
| 82² | 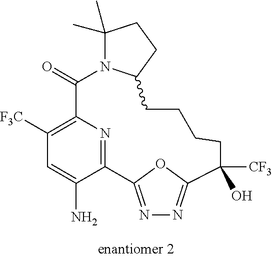 [SMILES: CC1(C)CCC2CCCC[C@](O)(C(F)(F)F)c3nnc(o3)-c3nc(c(C(F)(F)F)cc3N)C(=O)N21] | (6R)-20- Amino-6- hydroxy-14,14- dimethyl-6,18-bis (trifluoromethyl)- 22-oxa-3,4,15,21- tetraazatetracyclo [15.3.1.12, 5.011,15]doco sa- 1(21),2,4,17,1 9-pentaen-16-one (enantiomer 2) (Compound 115) |
| 83 | 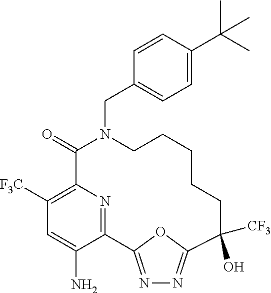 [SMILES: CC(C)(C)c1ccc(CN2CCCCC[C@](O)(C(F)(F)F)c3nnc(o3)-c3nc(c(C(F)(F)F)cc3N)C2=O)cc1] | (6R)-17- Amino-12-[(4- tert- butylphenyl) methyl]-6- hydroxy-6,15-bis (trifluoromethyl)- 19-oxa- 3,4,12,18- tetrazatricyclo [12.3.1.12,5] nonadeca- 1(18),2,4,14,1 6-pentaen-13-one (Compound 116) |
| 84 | 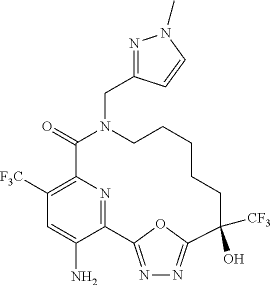 [SMILES: Cn1ccc(CN2CCCCC[C@](O)(C(F)(F)F)c3nnc(o3)-c3nc(c(C(F)(F)F)cc3N)C2=O)n1] | (6R)-17-Amino-6- hydroxy-12-[(1- methylpyrazol- 3-yl)methyl]- 6,15-bis (trifluorom ethyl)-19-oxa- 3,4,12,18- tetrazatricyclo [12.3.1.12,5] nonadeca- 1(18),2,4,14,1 6-pentaen-13-one (Compound 117) |
| 85 | 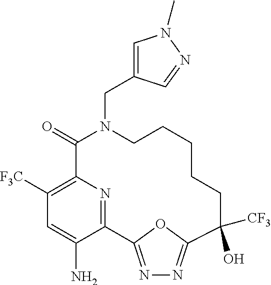 [SMILES: Cn1cc(CN2CCCCC[C@](O)(C(F)(F)F)c3nnc(o3)-c3nc(c(C(F)(F)F)cc3N)C2=O)cn1] | (6R)-17- Amino-6- hydroxy-12- [(1-methylpyrazol- 4-yl)methyl]- 6,15-bis (trifluoromethyl)- 19-oxa-3,4,12,18- tetrazatricyclo [12.3.1.12,5] nonadeca- 1(18),2,4,14,1 6-pentaen-13- one (Compound 118) |
| 86 | 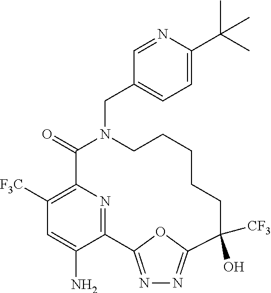 [SMILES: CC(C)(C)c1ccc(CN2CCCCC[C@](O)(C(F)(F)F)c3nnc(o3)-c3nc(c(C(F)(F)F)cc3N)C2=O)cn1] | (6R)-17- Amino-12-[(6- tert-butyl-3- pyridyl)methyl]- 6-hydroxy- 6,15- bis (trifluoromethyl)- 19-oxa- 3,4,12,18- tetrazatricyclo [12.3.1.12,5] nonadeca- 1(18),2,4,14,1 6-pentaen-13- one (Compound 119) |
| 87 | 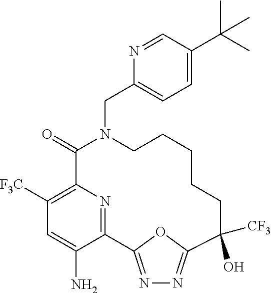 [SMILES: CC(C)(C)c1ccc(CN2CCCCC[C@](O)(C(F)(F)F)c3nnc(o3)-c3nc(c(C(F)(F)F)cc3N)C2=O)nc1] | (6R)-17-Amino-12-[(5- tert-butyl-2- pyridyl)methyl]- 6-hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 120) |
| 88 | 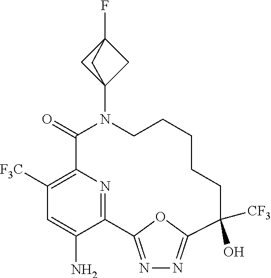 [SMILES: Nc1cc(C(F)(F)F)c2nc1-c1nnc(o1)[C@@](O)(C(F)(F)F)CCCCCN(C13CC(F)(C1)C3)C2=O] | (6R)-17- Amino-12-(3- fluoro-1- bicyclo[1.1.1] pentanyl)-6- hydroxy-6,15- bis (trifluorom ethyl)-19-oxa- 3,4,12,18- tetrazatricyclo [12.3.1.12,5] nonadeca- 1(18),2,4,14,1 6-pentaen-13-one (Compound 121) |
| 89 | 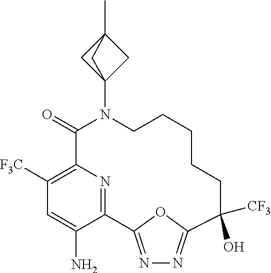 [SMILES: CC12CC(N3CCCCC[C@](O)(C(F)(F)F)c4nnc(o4)-c4nc(c(C(F)(F)F)cc4N)C3=O)(C1)C2] | (6R)-17-Amino-6- hydroxy-12- (3-methyl-1- bicyclo[1.1.1] pentanyl)-6,15-bis (trifluoromethyl)- 19-oxa- 3,4,12,18- tetrazatricyclo [12.3.1.12,5] nonadeca- 1(18),2,4,14,1 6-pentaen-13-one (Compound 122) |
| 90 | 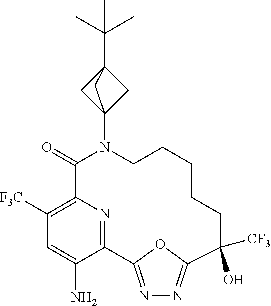 [SMILES: CC(C)(C)C12CC(N3CCCCC[C@](O)(C(F)(F)F)c4nnc(o4)-c4nc(c(C(F)(F)F)cc4N)C3=O)(C1)C2] | (6R)-17- Amino-12-(3- tert-butyl-1- bicyclo[1.1.1] pentanyl)-6- hydroxy-6,15- bis (trifluoromethyl)- 19-oxa-3,4,12,18- tetrazatricyclo [12.3.1.12,5] nonadeca- 1(18),2,4,14,1 6-pentaen-13- one (Compound 123) |
| 91 | 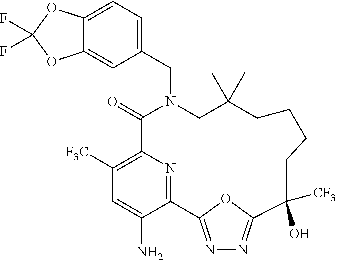 [SMILES: CC1(C)CCCC[C@](O)(C(F)(F)F)c2nnc(o2)-c2nc(c(C(F)(F)F)cc2N)C(=O)N(Cc2ccc3c(c2)OC(F)(F)O3)C1] | (6R)-18-amino-13- [(2,2-difluoro-1,3- benzodioxol- 5-yl)methyl]- 6-hydroxy-11,11- dimethyl-6,16-bis (trifluoromethyl)- 20-oxa-3,4,13,19- tetrazatricyclo [13.3.1.12,5]icosa- 1(19),2,4,15,1 7-pentaen-14-one (Compound 124) |
| 92 | 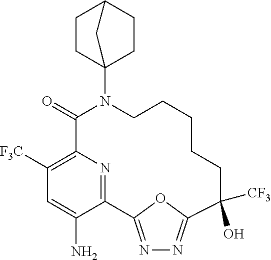 [SMILES: Nc1cc(C(F)(F)F)c2nc1-c1nnc(o1)[C@@](O)(C(F)(F)F)CCCCCN(C13CCC(CC1)C3)C2=O] | (6R)-17- amino-6- hydroxy-12- norbornan-1- yl-6,15-bis (trifluoromethyl)- 19-oxa- 3,4,12,18- tetrazatricyclo [12.3.1.12,5] nonadeca- 1(18),2,4,14,1 6-pentaen-13- one (Compound 125) |
| 93⁴ | 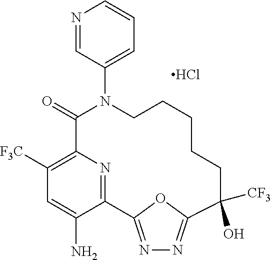 [SMILES: Nc1cc(C(F)(F)F)c2nc1-c1nnc(o1)[C@@](O)(C(F)(F)F)CCCCCN(c1cccnc1)C2=O ; Cl] | (6R)-17- amino-6- hydroxy-12- (3-pyridyl)- 6,15-bis (trifluoromethyl)- 19-oxa-3,4,12,18- tetrazatricyclo [12.3.1.12,5] nonadeca- 1(18),2,4,14,1 6-pentaen-13- one (hydrochloride salt) (Compound 126) |
| 94⁴ | 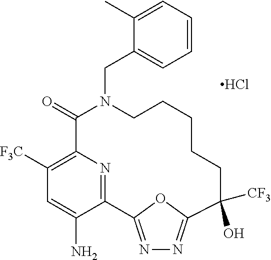 [SMILES: Cc1ccccc1CN1CCCCC[C@](O)(C(F)(F)F)c2nnc(o2)-c2nc(c(C(F)(F)F)cc2N)C1=O ; Cl] | (6R)-17- amino-6- hydroxy-12-(0- tolylmethyl)-6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 127) |
| 95⁴ | 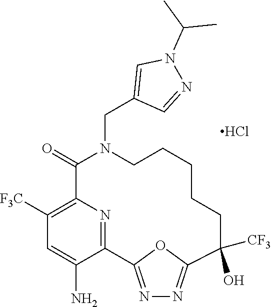 [SMILES: CC(C)n1cc(CN2CCCCC[C@](O)(C(F)(F)F)c3nnc(o3)-c3nc(c(C(F)(F)F)cc3N)C2=O)cn1 ; Cl] | (6R)-17- amino-6- hydroxy-12-[(1- isopropylpyrazol-4- yl)methyl]-6,1-bis (trifluoromethyl)- 19-oxa-3,4,12,18- tetrazatricyclo [12.3.1.12,5] nonadeca- 1(18),2,4,14,1 6-pentaen-13- one (hydrochloride salt) (Compound 128) |
| 96⁴ | 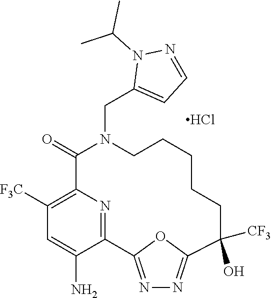 [SMILES: CC(C)n1nccc1CN1CCCCC[C@](O)(C(F)(F)F)c2nnc(o2)-c2nc(c(C(F)(F)F)cc2N)C1=O ; Cl] | (6R)-17- amino-6- hydroxy-12-[(2- isopropylpyrazol-3- yl)methyl]- 6,15-bis (trifluoromethyl)- 19-oxa- 3,4,12,18- tetrazatricyclo [12.3.1.12,5] nonadeca- 1(18),2,4,14,1 6-pentaen-13-one (hydrochloride salt) (Compound 129) |
| 97 | 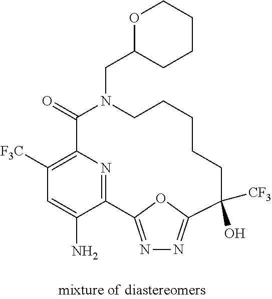 [SMILES: Nc1cc(C(F)(F)F)c2nc1-c1nnc(o1)[C@@](O)(C(F)(F)F)CCCCCN(CC1CCCCO1)C2=O] | (6R)-17- amino-6- hydroxy-12- (tetrahydropyran-2- ylmethyl)- 6,15- bis (trifluoromethyl)- 19-oxa- 3,4,12,18- tetrazatricyclo [12.3.1.12,5] nonadeca- 1(18),2,4,14,1 6-pentaen-13- one (mixture of diastereomers) (Compound 130) |
| 98 | 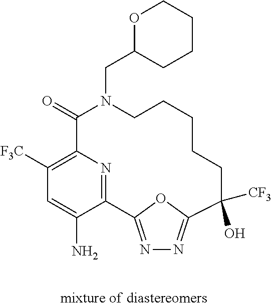 [SMILES: Nc1cc(C(F)(F)F)c2nc1-c1nnc(o1)[C@@](O)(C(F)(F)F)CCCCCN(CC1CCCCO1)C2=O] | (6R)-17- Amino-6- hydroxy-12- (tetrahydropyran-3- ylmethyl)- 6,15- bis (trifluoromethyl)- 19-oxa- 3,4,12,18- tetrazatricyclo [12.3.1.12,5] nonadeca- 1(18),2,4,14,1 6-pentaen-13- one (mixture of diastereomers) (Compound 131) |
| 99 | 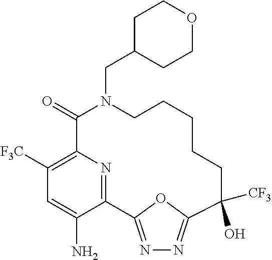 [SMILES: Nc1cc(C(F)(F)F)c2nc1-c1nnc(o1)[C@@](O)(C(F)(F)F)CCCCCN(CC1CCOCC1)C2=O] | (6R)-17-amino-6- hydroxy-12- (tetrahydropyran-4- ylmethyl)- 6,15-bis (trifluoromethyl)- 19-oxa-3,4,12,18- tetrazatricyclo [12.3.1.12,5] nonadeca- 1(18),2,4,14,1 6-pentaen-13-one (Compound 132) |
| 100 | 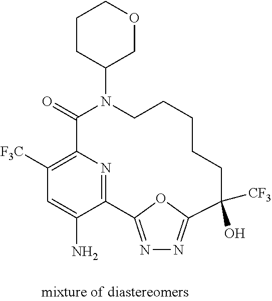 [SMILES: Nc1cc(C(F)(F)F)c2nc1-c1nnc(o1)[C@@](O)(C(F)(F)F)CCCCCN(C1CCCOC1)C2=O] | (6R)-17- Amino-6- hydroxy-12- tetrahydropyran- 3-yl-6,15-bis (trifluoromethyl)- 19-oxa- 3,4,12,18- tetrazatricyclo [12.3.1.12,5] nonadeca- 1(18),2,4,14,1 6-pentaen-13- one (mixture of diastereomers) (Compound 133) |
| 101 | 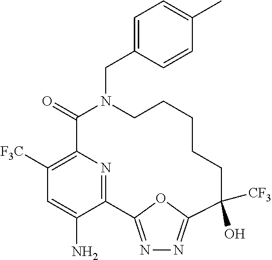 [SMILES: Cc1ccc(CN2CCCCC[C@](O)(C(F)(F)F)c3nnc(o3)-c3nc(c(C(F)(F)F)cc3N)C2=O)cc1] | (6R)-17-amino-6- hydroxy-12-(p- tolylmethyl)- 6,15-bis (trifluoromethyl)- 19-oxa- 3,4,12,18- tetrazatricyclo [12.3.1.12,5] nonadeca- 1(18),2,4,14,1 6-pentaen-13-one (Compound 134) |
| 102 | 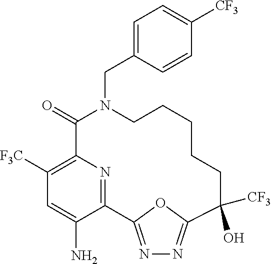 [SMILES: Nc1cc(C(F)(F)F)c2nc1-c1nnc(o1)[C@@](O)(C(F)(F)F)CCCCCN(Cc1ccc(C(F)(F)F)cc1)C2=O] | (6R)-17- amino-6- hydroxy-6,15-bis (trifluoromethyl)- 12-[[4- (trifluoromethyl) phenyl] methyl]-19-oxa- 3,4,12,18- tetrazatricyclo [12.3.1.12,5] nonadeca- 1(18),2,4,14,1 6-pentaen-13-one (Compound 135) |
| 103 | 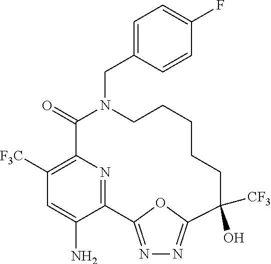 [SMILES: Nc1cc(C(F)(F)F)c2nc1-c1nnc(o1)[C@@](O)(C(F)(F)F)CCCCCN(Cc1ccc(F)cc1)C2=O] | (6R)-17- amino-12-[(4- fluorophenyl) methyl]-6- hydroxy-6,15-bis (trifluorom ethyl)-19-oxa- 3,4,12,18- tetrazatricyclo [12.3.1.12,5] nonadeca- 1(18),2,4,14,1 6-pentaen-13- one (Compound 13) |
| 104 | 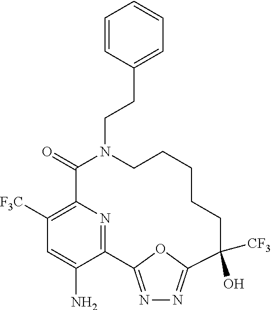 [SMILES: Nc1cc(C(F)(F)F)c2nc1-c1nnc(o1)[C@@](O)(C(F)(F)F)CCCCCN(CCc1ccccc1)C2=O] | (6R)-17- amino-6- hydroxy-12-(2- phenylethyl)- 6,15-bis (trifluoromethyl)- 19-oxa- 3,4,12,18- tetrazatricyclo [12.3.1.12,5] nonadeca- 1(18),2,4,14,1 6-pentaen-13-one (Compound 137) |
| 105 | 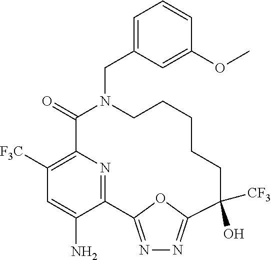 [SMILES: COc1cccc(CN2CCCCC[C@](O)(C(F)(F)F)c3nnc(o3)-c3nc(c(C(F)(F)F)cc3N)C2=O)c1] | (6R)-17- amino-6- hydroxy-12-[(3- methoxyphenyl) methyl]- 6,15-bis (trifluorom ethyl)-19-oxa- 3,4,12,18- tetrazatricyclo [12.3.1.12,5] nonadeca- 1(18),2,4,14,1 6-pentaen-13-one (Compound 138) |
| 106 | 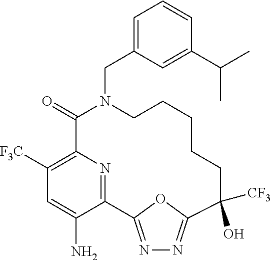 [SMILES: CC(C)c1cccc(CN2CCCCC[C@](O)(C(F)(F)F)c3nnc(o3)-c3nc(c(C(F)(F)F)cc3N)C2=O)c1] | (6R)-17-amino-6- hydroxy-12- [(3- isopropylphen yl)methyl]- 6,15-bis (trifluoromethyl)- 19-oxa- 3,4,12,18- tetrazatricyclo [12.3.1.12,5] nonadeca- 1(18),2,4,14,1 6-pentaen-13-one (Compound 139) |
| 107⁴ | 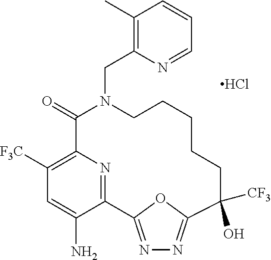 [SMILES: Cc1cccnc1CN1CCCCC[C@](O)(C(F)(F)F)c2nnc(o2)-c2nc(c(C(F)(F)F)cc2N)C1=O ; Cl] | (6R)-17-amino-6- hydroxy-12- [(3-methyl-2- pyridyl)methyl]- 6,15-bis (trifluoromethyl)- 19-oxa-3,4,12,18- tetrazatricyclo [12.3.1.12,5] nonadeca- 1(18),2,4,14,1 6-pentaen-13- one (hydrochloride salt) (Compound 140) |
| 108⁴ | 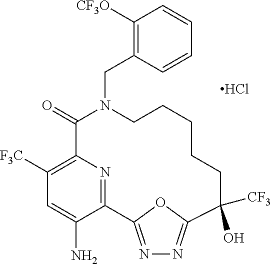 | (6R)-17-amino-6- hydroxy-12-[[2- (trifluoromethoxy) phenyl]methyl]-6,15- bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 141) |
| 109⁴ | 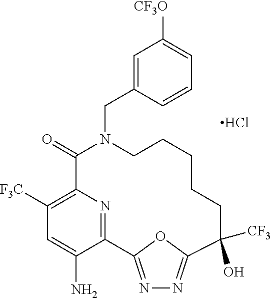 | (6R)-17-amino-6- hydroxy-12-[[3- (trifluoromethoxy) phenyl] methyl]-6,15- bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 142) |
| 110 | 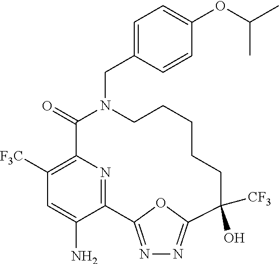 [SMILES: CC(C)Oc1ccc(CN2CCCCC[C@](O)(C(F)(F)F)c3nnc(o3)-c3nc(c(C(F)(F)F)cc3N)C2=O)cc1] | (6R)-17- amino-6- hydroxy-12-[(4- isopropoxyphenyl) methyl]-6,15-bis (trifluoromethyl)- 19-oxa-3,4,12,18- tetrazatricyclo [12.3.1.12,5] nonadeca- 1(18),2,4,14,1 6-pentaen-13-one (Compound 143) |
| 111 | 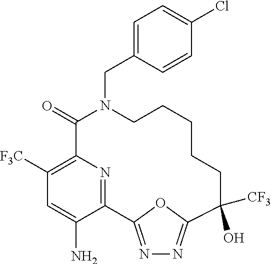 [SMILES: Nc1cc(C(F)(F)F)c2nc1-c1nnc(o1)[C@@](O)(C(F)(F)F)CCCCCN(Cc1ccc(Cl)cc1)C2=O] | (6R)-17- amino-12-[(4- chlorophenyl) methyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 144) |
| 112 | 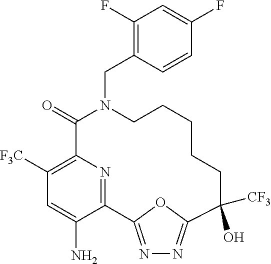 [SMILES: Nc1cc(C(F)(F)F)c2nc1-c1nnc(o1)[C@@](O)(C(F)(F)F)CCCCCN(Cc1ccc(F)cc1F)C2=O] | (6R)-17-amino-12- [(2,4- difluorophenyl) methyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 145) |
| 113 | 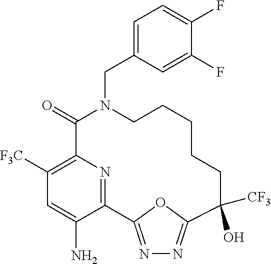 [SMILES: Nc1cc(C(F)(F)F)c2nc1-c1nnc(o1)[C@@](O)(C(F)(F)F)CCCCCN(Cc1ccc(F)c(F)c1)C2=O] | (6R)-17- amino-12-[(3,4- difluorophenyl) methyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 146) |
| 114 | 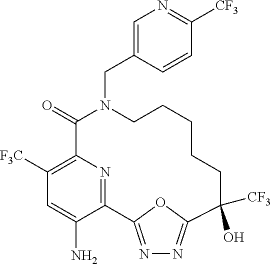 [SMILES: Nc1cc(C(F)(F)F)c2nc1-c1nnc(o1)[C@@](O)(C(F)(F)F)CCCCCN(Cc1ccc(C(F)(F)F)nc1)C2=O] | (6R)-17-amino-6- hydroxy-6,15- bis (trifluoromethyl)- 12-[[6- (trifluoromethyl)-3- pyridyl]methyl]- 19-oxa-3,4,12,18- tetrazatricyclo [12.3.1.12,5] nonadeca- 1(18),2,4,14,1 6-pentaen-13-one (Compound 147) |
| 115⁵ | 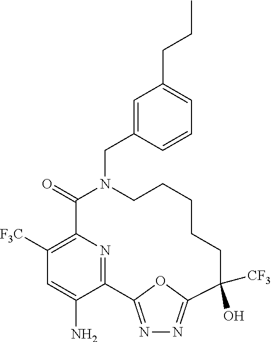 [SMILES: CCCc1cccc(CN2CCCCC[C@](O)(C(F)(F)F)c3nnc(o3)-c3nc(c(C(F)(F)F)cc3N)C2=O)c1] | (6R)-17-amino-6- hydroxy-12-[(3- propylphenyl) methyl]-6,15-bis (trifluoromethyl)- 19-oxa-3,4,12,18- tetrazatricyclo [12.3.1.12,5] nonadeca- 1(18),2,4,14,1 6-pentaen-13-one (Compound 148) |
| 116 | 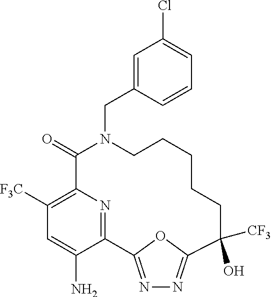 [SMILES: Nc1cc(C(F)(F)F)c2nc1-c1nnc(o1)[C@@](O)(C(F)(F)F)CCCCCN(Cc1cccc(Cl)c1)C2=O] | (6R)-17- amino-12-[(3- chlorophenyl) methyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (Compound 149) |
| 117 | 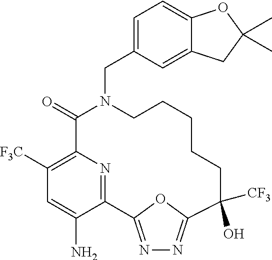 [SMILES: CC1(C)Cc2cc(CN3CCCCC[C@](O)(C(F)(F)F)c4nnc(o4)-c4nc(c(C(F)(F)F)cc4N)C3=O)ccc2O1] | (6R)-17- amino-12- [(2,2- dimethyl-3H- benzofuran-5- yl)methyl]-6- hydroxy-6,15- bis (trifluoromethyl)- 19-oxa-3,4,12,18- tetrazatricyclo [12.3.1.12,5] nonadeca- 1(18),2,4,14,1 6-pentaen-13-one (Compound 150) |
| 118 | 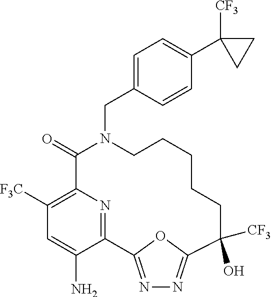 [SMILES: Nc1cc(C(F)(F)F)c2nc1-c1nnc(o1)[C@@](O)(C(F)(F)F)CCCCCN(Cc1ccc(C3(C(F)(F)F)CC3)cc1)C2=O] | (6R)-17- amino-6- hydroxy-6,15-bis (trifluoromethyl)- 12-[[4-[1- (trifluoromethyl) cyclopropyl lphenyl]methyl]- 19-oxa- 3,4,12,18 tetrazatricyclo [12.3.1.12,5] nonadeca- 1(18),2,4,14,1 6-pentaen-13-one (Compound 151) |
| 119 | 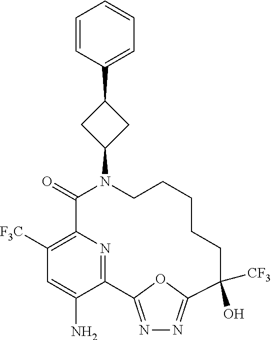 [SMILES: Nc1cc(C(F)(F)F)c2nc1-c1nnc(o1)[C@@](O)(C(F)(F)F)CCCCCN([C@H]1C[C@@H](c3ccccc3)C1)C2=O] | (6R)-17-amino-6- hydroxy-12-(3- phenylcyclobutyl)- 6,15-bis (trifluoromethyl)- 19-oxa-3,4,12,18- tetrazatricyclo [12.3.1.12,5] nonadeca- 1(18),2,4,14,1 6-pentaen-13-one (Compound 152) |
| 120⁴ | 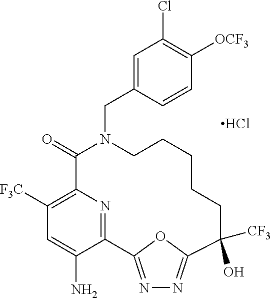 [SMILES: Nc1cc(C(F)(F)F)c2nc1-c1nnc(o1)[C@@](O)(C(F)(F)F)CCCCCN(Cc1ccc(OC(F)(F)F)c(Cl)c1)C2=O ; Cl] | (6R)-17-amino- 12-[[3-chloro-4- (trifluoromethoxy) phenyl] methyl]-6- hydroxy-6,15- bis(trifluoromethyl)- 19-oxa-3,4,12,18- tetrazatricyclo [12.3.1.12,5] nonadeca- 1(18),2,4,14,1 6-pentaen-13-one (hydrochloride salt) (Compound 153) |
| 121⁴ | 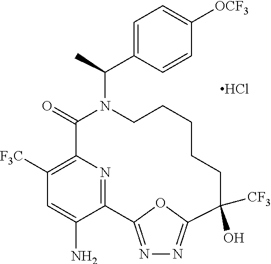 [SMILES: C[C@@H](c1ccc(OC(F)(F)F)cc1)N1CCCCC[C@](O)(C(F)(F)F)c2nnc(o2)-c2nc(c(C(F)(F)F)cc2N)C1=O ; Cl] | (6R)-17- amino-6- hydroxy-12- [(1S)-1-[4- (trifluoromethoxy) phenyl]ethyl]-6,15- bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 154) |
| 122⁴ | 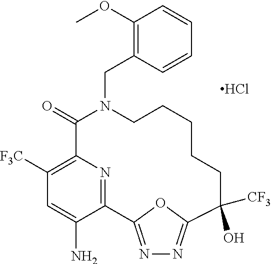 [SMILES: COc1ccccc1CN1CCCCC[C@](O)(C(F)(F)F)c2nnc(o2)-c2nc(c(C(F)(F)F)cc2N)C1=O ; Cl] | (6R)-17-amino-6- hydroxy-12-[(2- methoxyphenyl) methyl]-6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 155) |
| 123⁴ | 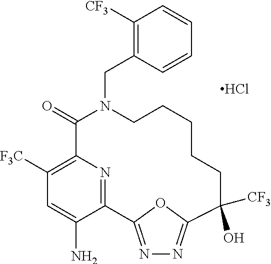 [SMILES: Nc1cc(C(F)(F)F)c2nc1-c1nnc(o1)[C@@](O)(C(F)(F)F)CCCCCN(Cc1ccccc1C(F)(F)F)C2=O ; Cl] | (6R)-17-amino-6- hydroxy-6,15-bis (trifluoromethyl)- 12-[[2- (trifluoromethyl) phenyl]methyl]- 19-oxa-3,4,12,18- tetrazatricyclo [12.3.1.12,5] nonadeca- 1(18),2,4,14,1 6-pentaen-13-one (hydrochloride salt) (Compound 156) |
| 124⁴ | 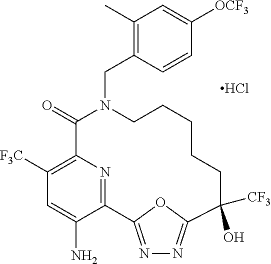 [SMILES: Cc1cc(OC(F)(F)F)ccc1CN1CCCCC[C@](O)(C(F)(F)F)c2nnc(o2)-c2nc(c(C(F)(F)F)cc2N)C1=O ; Cl] | (6R)-17-amino-6- hydroxy-12- [[2-methyl-4- (trifluoromethoxy) phenyl]methyl]- 6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 157) |
| 125⁴ | 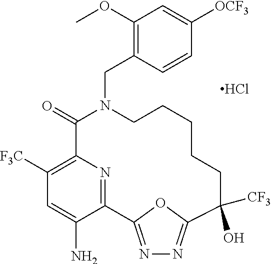 [SMILES: COc1cc(OC(F)(F)F)ccc1CN1CCCCC[C@](O)(C(F)(F)F)c2nnc(o2)-c2nc(c(C(F)(F)F)cc2N)C1=O ; Cl] | (6R)-17-amino-6- hydroxy-12- [[2-methoxy-4- (trifluoromethoxy) phenyl]methyl]-6,15- bis(trifluoromethyl)- 19-oxa-3,4,12,18- tetrazatricyclo [12.3.1.12,5] nonadeca- 1(18),2,4,14,1 6-pentaen-13- one (hydrochloride salt) (Compound 158) |
| 126⁴ | 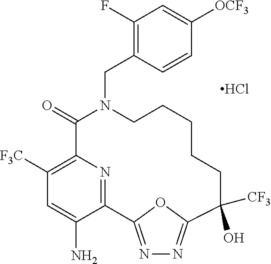 [SMILES: Nc1cc(C(F)(F)F)c2nc1-c1nnc(o1)[C@@](O)(C(F)(F)F)CCCCCN(Cc1ccc(OC(F)(F)F)cc1F)C2=O ; Cl] | (6R)-17- amino-12-[[2- fluoro-4- (trifluoromethoxy) phenyl]methyl]-6- hydroxy-6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 159) |
| 127⁴ | 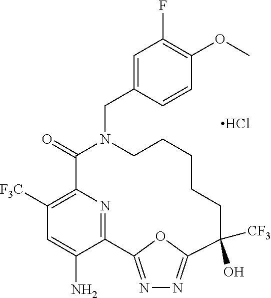 [SMILES: COc1ccc(CN2CCCCC[C@](O)(C(F)(F)F)c3nnc(o3)-c3nc(c(C(F)(F)F)cc3N)C2=O)cc1F ; Cl] | (6R)-17- amino-12-[(3- fluoro-4- methoxy- phenyl)methyl]- 6-hydroxy-6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 160) |
| 128⁴ | 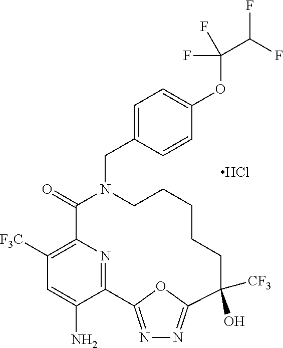 [SMILES: Nc1cc(C(F)(F)F)c2nc1-c1nnc(o1)[C@@](O)(C(F)(F)F)CCCCCN(Cc1ccc(OC(F)(F)C(F)F)cc1)C2=O ; Cl] | (6R)-17- amino-6- hydroxy-12- [[4-(1,1,2,2- tetrafluoroethoxy) phenyl]methyl]- 6,15-bis (trifluoromethyl)- 19-oxa- 3,4,12,18- tetrazatricyclo [12.3.1.12,5] nonadeca- 1(18),2,4,14,1 6-pentaen-13- one (hydrochloride salt) (Compound 161) |
| 129⁵ | 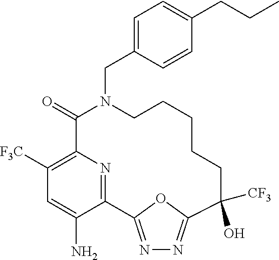 [SMILES: CCCc1ccc(CN2CCCCC[C@](O)(C(F)(F)F)c3nnc(o3)-c3nc(c(C(F)(F)F)cc3N)C2=O)cc1] | (6R)-17- amino-6- hydroxy-12-[(4- propylphenyl) methyl]-6,15-bis (trifluoromethyl)- 19-oxa- 3,4,12,18- tetrazatricyclo [12.3.1.12,5] nonadeca- 1(18),2,4,14,1 6-pentaen-13- one (Compound 162) |
| 130 | 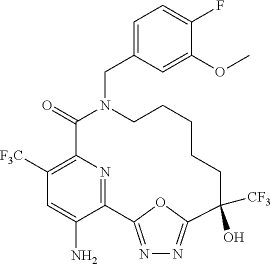 [SMILES: COc1cc(CN2CCCCC[C@](O)(C(F)(F)F)c3nnc(o3)-c3nc(c(C(F)(F)F)cc3N)C2=O)ccc1F] | (6R)-17- amino-12-[(4- fluoro-3-methoxy- phenyl)methyl]- 6-hydroxy-6,15- bis (trifluoromethyl)- 19-oxa-3,4,12,18- tetrazatricyclo [12.3.1.12,5] nonadeca- 1(18),2,4,14,1 6-pentaen-13-one (Compound 163) |
| 131 | 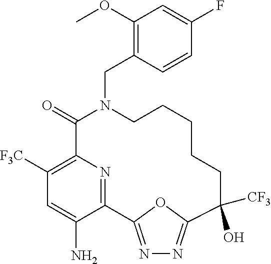 [SMILES: COc1cc(F)ccc1CN1CCCCC[C@](O)(C(F)(F)F)c2nnc(o2)-c2nc(c(C(F)(F)F)cc2N)C1=O] | (6R)-17- amino-12-[(4- fluoro-2-methoxy- phenyl)methyl]- 6-hydroxy-6,15-bis (trifluoromethyl)- 19-oxa- 3,4,12,18- tetrazatricyclo [12.3.1.12,5] nonadeca- 1(18),2,4,14,1 6-pentaen-13-one (Compound 164) |
| 132⁴ | 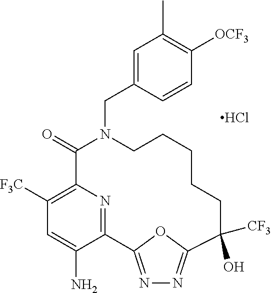 [SMILES: Cc1cc(CN2CCCCC[C@](O)(C(F)(F)F)c3nnc(o3)-c3nc(c(C(F)(F)F)cc3N)C2=O)ccc1OC(F)(F)F ; Cl] | (6R)-17- amino-6- hydroxy-12- [3-methyl-4- (trifluoromethoxy) phenyl] methyl]-6,15-bis (trifluoromethyl)- 19-oxa- 3,4,12,18- tetrazatricyclo [12.3.1.12,5] nonadeca- 1(18),2,4,14,1 6-pentaen-13-one (hydrochloride salt) (Compound 165) |
| 133 | 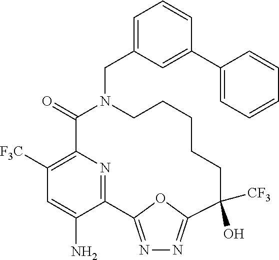 [SMILES: Nc1cc(C(F)(F)F)c2nc1-c1nnc(o1)[C@@](O)(C(F)(F)F)CCCCCN(Cc1cccc(-c3ccccc3)c1)C2=O] | (6R)-17- amino-6- hydroxy-12-[(3- phenylphenyl) methyl]-6,15-bis (trifluoromethyl)- 19-oxa- 3,4,12,18- tetrazatricyclo [12.3.1.12,5] nonadeca- 1(18),2,4,14,1 6-pentaen-13- one (Compound 166) |
| 134 | 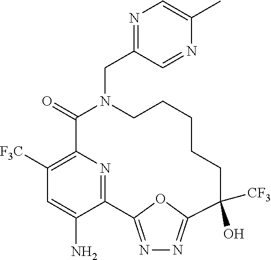 [SMILES: Cc1cnc(CN2CCCCC[C@](O)(C(F)(F)F)c3nnc(o3)-c3nc(c(C(F)(F)F)cc3N)C2=O)cn1] | (6R)-- 17- amino-6- hydroxy-12-[(5- methylpyrazin- 2-yl)methyl]- 6,15-bis (trifluoromethyl)- 19-oxa- 3,4,12,18- tetrazatricyclo [12.3.1.12,5] nonadeca- 1(18),2,4,14,1 6-pentaen-13- one (Compound 167) |
| 135^{4,5} | 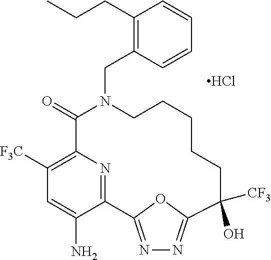 [SMILES: CCCc1ccccc1CN1CCCCC[C@](O)(C(F)(F)F)c2nnc(o2)-c2nc(c(C(F)(F)F)cc2N)C1=O ; Cl] | (6R)-17- amino-6- hydroxy-12-[(2- propylphenyl) methyl]-6,15-bis (trifluoromethyl)- 19-oxa- 3,4,12,18- tetrazatricyclo [12.3.1.12,5] nonadeca- 1(18),2,4,14,1 6-pentaen-13- one (hydrochloride salt) (Compound 168) |
| 136 | 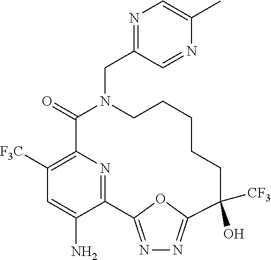 [SMILES: Cc1cnc(CN2CCCCC[C@](O)(C(F)(F)F)c3nnc(o3)-c3nc(c(C(F)(F)F)cc3N)C2=O)cn1] | (6R)-17-amino-6- hydroxy-12-[(5- methylpyrimidin-2- yl)methyl]- 6,15- bis (trifluoromethyl)- 19-oxa-3,4,12,18- tetrazatricyclo [12.3.1.12,5] nonadeca- 1(18),2,4,14,1 6-pentaen-13-one (Compound 169) |
| 137 | 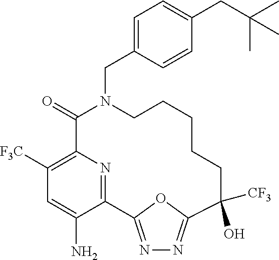 [SMILES: CC(C)(C)Cc1ccc(CN2CCCCC[C@](O)(C(F)(F)F)c3nnc(o3)-c3nc(c(C(F)(F)F)cc3N)C2=O)cc1] | (6R)-17- amino-12-[[4-(2,2- dimethylpropyl) phenyl] methyl]-6-hydroxy- 6,15-bis (trifluoromethyl)- 19-oxa-3,4,12,18- tetrazatricyclo [12.3.1.12,5] nonadeca- 1(18),2,4,14,1 6-pentaen-13-one (Compound 170) |
| 138 | 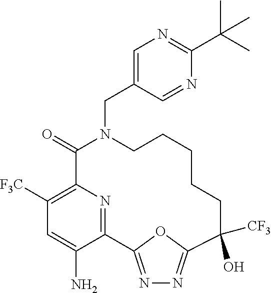 [SMILES: CC(C)(C)c1ncc(CN2CCCCC[C@](O)(C(F)(F)F)c3nnc(o3)-c3nc(c(C(F)(F)F)cc3N)C2=O)cn1] | (6R)-17- amino-12-[(2- tert- butylpyrimidin-5- yl)methyl]-6- hydroxy-6,15- bis (trifluoromethyl)- 19-oxa- 3,4,12,18- tetrazatricyclo [12.3.1.12,5] nonadeca- 1(18),2,4,14,1 6-pentaen-13-one (Compound 171) |
| 139 | 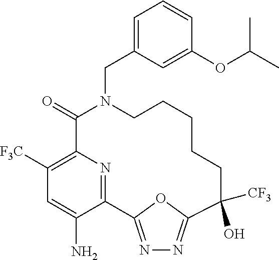 [SMILES: CC(C)Oc1cccc(CN2CCCCC[C@](O)(C(F)(F)F)c3nnc(o3)-c3nc(c(C(F)(F)F)cc3N)C2=O)c1] | (6R)-17- amino-6- hydroxy-12- [(3- isopropoxyphenyl) methyl]-6,15-bis (trifluoromethyl)- 19-oxa-3,4,12,18- tetrazatricyclo [12.3.1.12,5] nonadeca- 1(18),2,4,14,1 6-pentaen-13-one (Compound 172) |
| 140 | 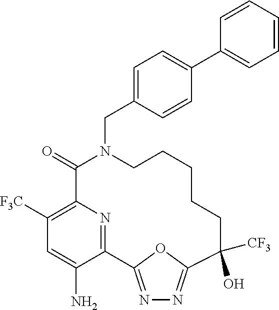 [SMILES: Nc1cc(C(F)(F)F)c2nc1-c1nnc(o1)[C@@](O)(C(F)(F)F)CCCCCN(Cc1ccc(-c3ccccc3)cc1)C2=O] | (6R)-17- amino-6- hydroxy-12- [(4- phenylphenyl) methyl]-6,15- bis (trifluoromethyl)- 19-oxa-3,4,12,18- tetrazatricyclo [12.3.1.12,5] nonadeca- 1(18),2,4,14,1 6-pentaen-13-one (Compound 173) |
| 141 | 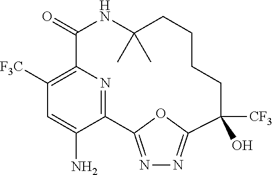 [SMILES: CC1(C)CCCC[C@](O)(C(F)(F)F)c2nnc(o2)-c2nc(c(C(F)(F)F)cc2N)C(=O)N1] | (6R)-17-amino-6- hydroxy-11,11- dimethyl-6,15-bis (trifluoromethyl)- 19-oxa-3,4,12,18- tetrazatricyclo [12.3.1.12,5] nonadeca- 1(18),2,4,14,1 6-pentaen-13-one (Compound 174) |
| 142 | 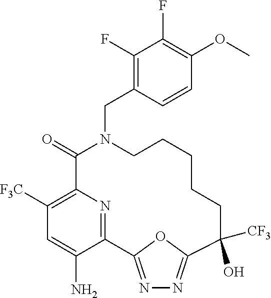 [SMILES: COc1ccc(CN2CCCCC[C@](O)(C(F)(F)F)c3nnc(o3)-c3nc(c(C(F)(F)F)cc3N)C2=O)c(F)c1F] | (6R)-17- amino-12- [(2,3-difluoro- 4-methoxy- phenyl)methyl]- 6-hydroxy-6,15- bis (trifluoromethyl)- 19-oxa- 3,4,12,18- tetrazatricyclo [12.3.1.12,5] nonadeca- 1(18),2,4,14,1 6-pentaen-13- one (Compound 175) |
| 143 | 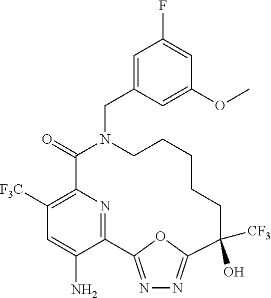 [SMILES: COc1cc(F)cc(CN2CCCCC[C@](O)(C(F)(F)F)c3nnc(o3)-c3nc(c(C(F)(F)F)cc3N)C2=O)c1] | (6R)-17- amino-12-[(3- fluoro-5- methoxy- phenyl)methyl]- 6-hydroxy-6,5- bis (trifluoromethyl)- 19-oxa-3,4,12,18- tetrazatricyclo [12.3.1.12,5] nonadeca- 1(18),2,4,14,1 6-pentaen-13-one (Compound 176) |
| 144 | 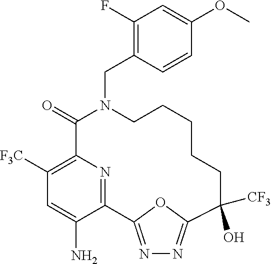 [SMILES: COc1ccc(CN2CCCCC[C@](O)(C(F)(F)F)c3nnc(o3)-c3nc(c(C(F)(F)F)cc3N)C2=O)c(F)c1] | (6R)-17- amino-12-[(2- fluoro-4- methoxy- phenyl)methyl]- 6-hydroxy-6,15-bis (trifluorom ethyl)-19-oxa- 3,4,12,18- tetrazatricyclo [12.3.1.12,5] nonadeca- 1(18),2,4,14,1 6-pentaen-13- one (Compound 177) |
| 145⁴ | 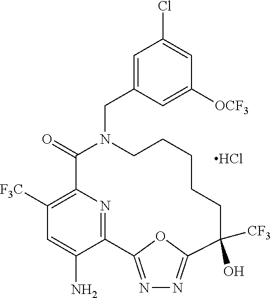 [SMILES: Nc1cc(C(F)(F)F)c2nc1-c1nnc(o1)[C@@](O)(C(F)(F)F)CCCCCN(Cc1cc(Cl)cc(OC(F)(F)F)c1)C2=O ; Cl] | (6R)-17- amino-12-[[3- chloro-5- (trifluoromethoxy) phenyl]methyl]-6- hydroxy-6,15- bis (trifluoromethyl)- 19-oxa- 3,4,12,18- tetrazatricyclo [12.3.1.12,5] nonadeca- 1(18),2,4,14,1 6-pentaen-13- one (Compound 178) |
| 146⁴ | 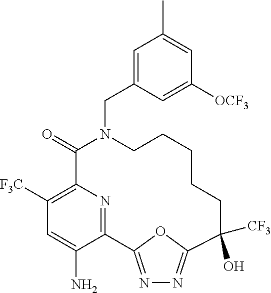 [SMILES: Cc1cc(CN2CCCCC[C@](O)(C(F)(F)F)c3nnc(o3)-c3nc(c(C(F)(F)F)cc3N)C2=O)cc(OC(F)(F)F)c1] | (6R)-17-amino-6- hydroxy-12- [[3-methyl-5- (trifluoromethoxy) phenyl]methyl]- 6,15-bis (trifluoromethyl)- 19-oxa-3,4,12,18- tetrazatricyclo [12.3.1.12,5] nonadeca- 1(18),2,4,14,1 6-pentaen-13-one (Compound 179) |
| 147 | 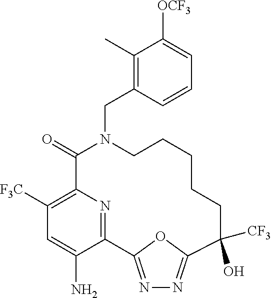 [SMILES: Cc1c(CN2CCCCC[C@](O)(C(F)(F)F)c3nnc(o3)-c3nc(c(C(F)(F)F)cc3N)C2=O)cccc1OC(F)(F)F] | (6R)-17- amino-6- hydroxy-12- [[2-methyl-3- (trifluoromethoxy) phenyl]methyl]-6,15- bis(trifluoromethyl)- 19-oxa-3,4,12,18- tetrazatricyclo [12.3.1.12,5] nonadeca- 1(18),2,4,14,1 6-pentaen-13-one (Compound 180) |
| 148 | 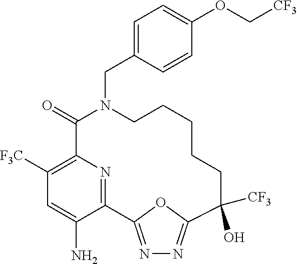 [SMILES: Nc1cc(C(F)(F)F)c2nc1-c1nnc(o1)[C@@](O)(C(F)(F)F)CCCCCN(Cc1ccc(OCC(F)(F)F)cc1)C2=O] | (6R)-17-amino-6- hydroxy-12- [[4-(2,2,2- trifluoroethoxy) phenyl]methyl]- 6,15-bis (trifluoromethyl)- 19-oxa-3,4,12,18- tetrazatricyclo [12.3.1.12,5] nonadeca- 1(18),2,4,14,1 6-pentaen-13-one (Compound 181) |
| 149 | 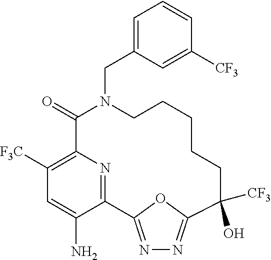 [SMILES: Nc1cc(C(F)(F)F)c2nc1-c1nnc(o1)[C@@](O)(C(F)(F)F)CCCCCN(Cc1cccc(C(F)(F)F)c1)C2=O] | (6R)-17-amino-6- hydroxy-6,15-bis (trifluoromethyl)- 12-[[3- (trifluoromethyl) phenyl]methyl]- 19-oxa-3,4,12,18- tetrazatricyclo [12.3.1.12,5] nonadeca- 1(18),2,4,14,1 6-pentaen-13-one (Compound 182) |
| 150⁶ | 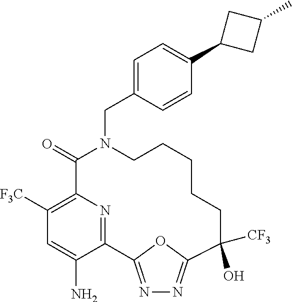 [SMILES: C[C@H]1C[C@H](c2ccc(CN3CCCCC[C@](O)(C(F)(F)F)c4nnc(o4)-c4nc(c(C(F)(F)F)cc4N)C3=O)cc2)C1] | (6R)-17-amino-6- hydroxy-12- ({4-[trans-3- methylcyclobutyl] phenyl} methyl)-6,15-bis (trifluoromethyl)- 19-oxa-3,4,12,18- tetraazatricyclo [12.3.1.12,5] nonadeca- 1(18),2,4,14,1 6-pentaen-13-one (Compound 183) |
| 151 | 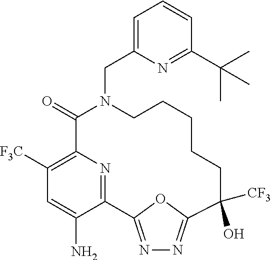 [SMILES: CC(C)(C)c1cccc(CN2CCCCC[C@](O)(C(F)(F)F)c3nnc(o3)-c3nc(c(C(F)(F)F)cc3N)C2=O)n1] | (6R)-17- amino-12-[(6- tert-butyl-2- pyridyl)methyl]- 6-hydroxy- 6,15-bis (trifluoromethyl)- 19-oxa-3,4,12,18- tetrazatricyclo [12.3.1.12,5] nonadeca- 1(18),2,4,14,1 6-pentaen-13-one (Compound 184) |
| 152⁴ | 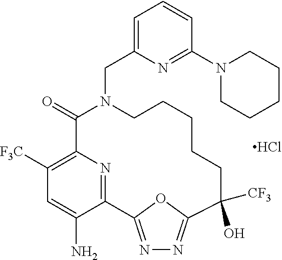 [SMILES: Nc1cc(C(F)(F)F)c2nc1-c1nnc(o1)[C@@](O)(C(F)(F)F)CCCCCN(Cc1cccc(N3CCCCC3)n1)C2=O ; Cl] | (6R)-17-amino-6- hydroxy-12- [[6-(1- piperidyl)-2- pyridyl]methyl]- 6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 185) |
| 153 | 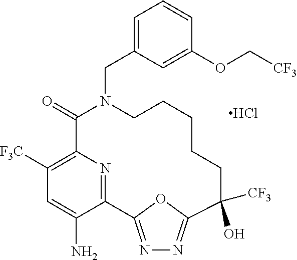 [SMILES: Nc1cc(C(F)(F)F)c2nc1-c1nnc(o1)[C@@](O)(C(F)(F)F)CCCCCN(Cc1cccc(OCC(F)(F)F)c1)C2=O ; Cl] | (6R)-17- amino-6- hydroxy-12- [[3-(2,2,2- trifluoroethoxy) phenyl]methyl]- 6,15-bis (trifluoromethyl)- 19-oxa-3,4,12,18- tetrazatricyclo [12.3.1.12,5] nonadeca- 1(18),2,4,14,1 6-pentaen-13-one (hydrochloride salt) (Compound 186) |
| 154 | 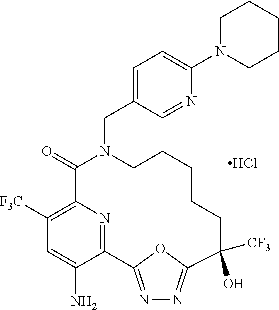 [SMILES: Nc1cc(C(F)(F)F)c2nc1-c1nnc(o1)[C@@](O)(C(F)(F)F)CCCCCN(Cc1ccc(N3CCCCC3)nc1)C2=O ; Cl] | (6R)-17-amino-6- hydroxy-12-[[6-(1- piperidyl)-3- pyridyl]methyl]- 6,15-bis (trifluoromethyl)- 19-oxa- 3,4,12,18- tetrazatricyclo [12.3.1.12,5] nonadeca- 1(18),2,4,14,1 6-pentaen-13- one (hydrochloride salt) (Compound 187) |

¹final product purified by preparative HPLC eluting with a gradient from 1% to 80% acetonitrile in 5 mM aqueous HCl at 50 mL/min over 14 min through a Luna 5 μM C18 100 Å 75 × 30 mm column
²compounds were prepared by Method 13, however the diastereomeric products resulting from step 3 were separated during the reverse-phase HPLC purification and each was taken into step 4 separately
³triisopropylsilane was not used in step 4 of Method 13
⁴final product purified by preparative HPLC eluting with a gradient of 1% to 99% acetonitrile in 5 mM aqueous HCl
⁵cyclopropyl group present in starting amine opened to the n-propyl group during step 3 of Method 13
⁶bicyclo[1.1.1]pentane group present in starting amine opened to the trans-3-methyl cyclobutaneduring step 3 of Method 13
⁷purified by reverse-phase preparative chromatography using a C18 column and a gradient eluent of 1% to 50% acetonitrile in water containing 5 mM HCl
⁸purified by reverse-phase preparative chromatography using a C18 column and a gradient eluent of 1% to 30% acetonitrile in water containing 5 mM HCl

TABLE 4
Characterization Data and chiral SFC Conditions (if
applicable) for Final Compounds Prepared by Method 13

| | | | SFC conditions to |
|---|---|---|---|
| Example | LCMS data and | | separate enantiomers |
| number | method | NMR data (shifts in ppm) | from racemic final product |
| 41 | ESI-MS m/z calc. | ¹H HMR (400 MHz, CDCl₃) δ 7.54 | Chiral SFC using an AD |
| | 529.15485, found | (d, J = 4.3 Hz, 1H), 7.31 (q, J = 7.5, | column (250 × 21.2 |
| | 530.1 (M + 1)⁺; | 6.7 Hz, 5H), 5.96 (s, 2H), 5.38 (d, J = | mm, 5 μm particle size) |
| | Retention time: | 14.6 Hz, 1H), 4.28 (d, J = 13.9 | and a gradient of 10 to |
| | 1.43 minutes (LC | Hz, 1H), 3.74 (s, 1H), 3.07 (d, J = | 70% MeOH (5 mM |
| | Method A) | 12.6 Hz, 1H), 3.00-2.85 (m, 1H), | NH₃) and CO₂ over 10 |
| | | 2.53 (d, J = 19.9 Hz, 2H), 2.26 (t, J = | minutes using a flow |
| | | 11.8 Hz, 1H), 1.81 (d, J = 38.1 | rate of 60 mL/min. First |
| | | Hz, 3H), 1.15 (s, 1H), 0.65 (s, 1H); | enantiomer to elute |
| | | ¹⁹F NMR (376 MHz, CDCl₃) | |
| | | δ −60.21 (d, J = 3.1 Hz), −80.17. | |
| 42 | ESI-MS m/z calc. | ¹H HMR (400 MHz, CDCl₃) δ 7.54 | Chiral SFC using an AD |
| | 529.15485, found | (s, 1H), 7.39-7.28 (m, 5H), 5.96 (s, | column (250 × 21.2 |
| | 530.1 (M + 1)⁺; | 2H), 5.38 (d, J = 15.0 Hz, 1H), 4.28 | mm, 5 μm particle size) |
| | Retention time: | (d, J = 15.0 Hz, 1H), 3.71 (s, 1H), | and a gradient of 10 to |
| | 1.43 minutes (LC | 3.09 (t, J = 12.8 Hz, 1H), 3.02- | 70% MeOH (5 mM |
| | Method A) | 2.88 (m, 1H), 2.53 (m, 2H), 2.26 | NH₃) and CO₂ over 10 |
| | | (m, 1H), 1.93-1.72 (m, 2H), 1.70- | minutes using a flow |
| | | 1.61 (m, 1H), 1.21-1.08 (m, 1H), | rate of 60 mL/min. |
| | | 0.72-0.56 (m, 1H); ¹⁹F NMR (376 | Second enantiomer to |
| | | MHz, CDCl₃) δ −60.21, −80.17. | elute |
| 43 | ESI-MS m/z calc. | ¹H HMR (400 MHz, CDCl₃) δ 7.53 | Chiral SFC using AD |
| | 559.1654, found | (s, 1H), 7.30-7.21 (m, 2H), 6.87 | column (250 × 21.2 |
| | 560.3 (M + 1)⁺; | (d, J = 8.6 Hz, 2H), 5.96 (s, 2H), | mm, 5 μm particle size) |
| | Retention time: | 5.30 (s, 2H), 4.20 (d, J = 14.7 Hz, | and a gradient of 10 to |
| | 1.4 minutes (LC | 1H), 3.80 (s, 3H), 3.12-2.98 (m, | 70% MeOH (5 mM |
| | Method A) | 1H), 3.00-2.84 (m, 1H), 2.50 (q, J = | NH₃) and CO₂ over 10 |
| | | 12.9 Hz, 2H), 2.26 (m, 1H), 1.82 | minutes using a flow |
| | | (m, 2H), 1.70-1.60 (m, 1H), 1.15 | rate of 60 mL/min. First |
| | | (m, 1H), 0.66 (m, 1H); ¹⁹F NMR | enantiomer to elute |
| | | (376 MHz, Chloroform-d) δ −60.20 | |
| | | (d, J = 2.0 Hz), −80.15. | |
| 44 | ESI-MS m/z calc. | ¹H HMR (400 MHz, CDCl₃) δ 7.53 | Chiral SFC using AD |
| | 559.1654, found | (s, 1H), 7.29-7.22 (m, 2H), 6.91- | column (250 × 21.2 |
| | 560.1 (M + 1)⁺; | 6.80 (m, 2H), 5.96 (s, 2H), 5.33 (s, | mm, 5 μm particle size) |
| | Retention time: | 1H), 5.30 (s, 1H), 4.20 (d, J = 14.7 | and a gradient of 10 to |
| | 1.4 minutes (LC | Hz, 1H), 3.80 (s, 3H), 3.15-2.99 | 70% MeOH (5 mM |
| | Method A) | (m, 1H), 3.00-2.85 (m, 1H), 2.51 | NH₃) and CO₂ over 10 |
| | | (m, 2H), 2.26 (m, 1H), 1.90-1.72 | minutes using a flow |
| | | (m, 2H), 1.64 (dd, J = 7.8, 3.8 Hz, | rate of 60 mL/min. |
| | | 1H), 1.15 (m, 1H), 0.75-0.56 (m, | Second enantiomer to |
| | | 1H); ¹⁹F NMR (376 MHz, CDCl₃) | elute |
| | | δ −60.07, −80.18. | |
| 45 | ESI-MS m/z calc. | ¹H HMR (400 MHz, CD₃OD) δ | Chiral SFC using AD |
| | 493.15485, found | 7.73 (s, 1H), 4.57 (m, 1H), 3.25- | column (250 × 21.2 |
| | 494.1 (M + 1)⁺; | 3.08 (m, 2H), 2.48-2.21 (m, 7H), | mm, 5 μm particle size) |
| | Retention time: | 1.80 (m, 4H), 1.73-1.59 (m, 1H), | and a gradient of 10 to |
| | 1.34 minutes (LC | 1.19 (m, 1H), 0.93 (d, J = 17.4 Hz, | 70% MeOH (5 mM |
| | Method A) | 1H); ¹⁹F NMR (376 MHz, CD₃OD) | NH₃) and CO₂ over 10 |
| | | δ −61.82, −81.17. | minutes. First |
| | | | enantiomer to elute |
| 46 | ESI-MS m/z calc. | ¹H HMR (400 MHz, CD₃OD) δ | Chiral SFC using AD |
| | 493.15485, found | 7.73 (s, 1H), 4.67-4.49 (m, 1H), | column (250 × 21.2 |
| | 494.1 (M + 1)⁺; | 3.28-3.09 (m, 2H), 2.52-2.16 (m, | mm, 5 μm particle size) |
| | Retention time: | 7H), 1.94-1.76 (m, 4H), 1.75- | and a gradient of 10 to |
| | 1.35 minutes (LC | 1.60 (m, 1H), 1.19 (m, 1H), 0.91 (s, | 70% MeOH (5 mM |
| | Method A) | 1H); ¹⁹F NMR (376 MHz, CD₃OD) | NH₃) and CO₂ over 10 |
| | | δ −61.82, −81.15. | minutes. Second |
| | | | enantiomer to elute |
| 47 | ESI-MS m/z calc. | ¹H HMR (400 MHz, CDCl₃) δ 7.48 | |
| | 509.18616, found | (s, 1H), 5.90 (s, 2H), 4.16 (s, 1H), | |
| | 510.1 (M + 1)⁺; | 3.86 (d, J = 13.6 Hz, 1H), 3.18 (m, | |
| | Retention time: | 1H), 3.13-3.04 (m, 1H), 2.94 (d, J = | |
| | 1.51 minutes (LC | 13.6 Hz, 1H), 2.56-2.38 (m, 2H), | |
| | Method A) | 2.33-2.21 (m, 1H), 1.87 (d, J = | |
| | | 12.0 Hz, 2H), 1.68 (s, 1H), 1.19- | |
| | | 1.05 (m, 1H), 1.02 (s, 9H), 0.64 (s, | |
| | | 1H); ¹⁹F NMR (376 MHz, CDCl₃) | |
| | | δ −60.24, −79.93 (d, J = 32.0 Hz). | |
| 48 | ESI-MS m/z calc. | ¹H HMR (400 MHz, CDCl₃) δ 7.50 | |
| | 521.18616, found | (s, 1H), 5.90 (s, 2H), 4.22 (t, J = | |
| | 522.1 (M + 1)⁺; | 12.1 Hz, 1H), 3.80 (s, 1H), 3.07 (m, | |
| | Retention time: | 1H), 2.92 (t, J = 12.8 Hz, 1H), 2.55 | |
| | 1.51 minutes (LC | (t, J = 12.9 Hz, 2H), 2.24 (dd, J = | |
| | Method A) | 15.1, 8.0 Hz, 1H), 1.99 (d, J = 12.0 | |
| | | Hz, 1H), 1.83 (t, J = 15.1 Hz, 4H), | |
| | | 1.73-1.60 (m, 4H), 1.43 (m, 2H), | |
| | | 1.22-1.02 (m, 2H), 0.87 (d, J = | |
| | | 11.4 Hz, 1H), 0.50 (s, 1H); ¹⁹F | |
| | | NMR (376 MHz, CDCl₃) | |
| | | δ −60.46, −80.31. | |
| 49 | ESI-MS m/z calc. | ¹H HMR (400 MHz, CDCl₃) δ 7.50 | |
| | 481.15485, found | (s, 1H), 5.94 (s, 2H), 3.96 (s, 1H), | |
| | 482.1 (M + 1)⁺; | 3.71 (m, 1H), 3.30-3.21 (m, 1H), | |
| | Retention time: | 3.20-3.10 (m, 1H), 3.09-2.96 (m, | |
| | 1.28 minutes (LC | 1H), 2.58-2.39 (m, 2H), 2.27 (m, | |
| | Method A) | 1H), 1.94-1.74 (m, 2H), 1.70 (m, | |
| | | 2H), 1.32-1.15 (m, 2H), 0.96 (t, J = | |
| | | 7.4 Hz, 3H), 0.74 (m, 1H); ¹⁹F | |
| | | NMR (376 MHz, CDCl₃) | |
| | | δ −60.44, −80.08. | |
| 50 | ESI-MS m/z calc. | ¹H HMR (400 MHz, CDCl₃) δ 7.51 | |
| | 493.15485, found | (s, 1H), 5.94 (s, 2H), 4.02-3.90 (m, | |
| | 494.1 (M + 1)⁺; | 1H), 3.78 (dd, J = 13.9, 6.6 Hz, | |
| | Retention time: | 1H), 3.26-3.11 (m, 3H), 2.57- | |
| | 1.31 minutes (LC | 2.39 (m, 2H), 2.27 (m, 1H), 1.99- | |
| | Method A) | 1.62 (m, 2H), 1.29-1.08 (m, 3H), | |
| | | 0.74 (m 1H), 0.66-0.46 (m, 2H), | |
| | | 0.42-0.29 (m, 2H); ¹⁹F NMR (376 | |
| | | MHz, CDCl₃) δ −60.44, −80.09. | |
| 51 | ESI-MS m/z calc. | ¹H HMR (400 MHz, CD₃OD) δ | |
| | 529.136, found | 7.75 (s, 1H), 4.26 (m, 1H), 3.30- | |
| | 530.1 (M + 1)⁺; | 2.88 (m, 6H), 2.40 (s, 2H), 2.30- | |
| | Retention time: | 2.16 (m, 1H), 1.90-1.77 (m, 2H), | |
| | 1.31 minutes (LC | 1.74-1.58 (m, 1H), 1.28-1.17 (m, | |
| | Method A) | 1H), 0.97 (s, 1H); ¹⁹F NMR (376 | |
| | | MHz, CD₃OD) | |
| | | δ −61.80, −81.12, −84.96 (d, J = | |
| | | 198.9 Hz), −102.32 (d, J = 199.0 Hz). | |
| 52 | ESI-MS m/z calc. | ¹H HMR (400 MHz, CD₃OD) δ | |
| | 495.1705, found | 7.60 (s, 1H), 3.10-2.95 (m, 1H), | |
| | 496.1 (M + 1)⁺; | 2.80 (t, J = 13.7 Hz, 1H), 2.66- | |
| | Retention time: | 2.47 (m, 1H), 2.45-2.23 (m, 1H), | |
| | 1.38 minutes (LC | 2.19-1.97 (m, 1H), 1.95-1.62 (m, | |
| | Method A) | 1H), 1.48 (s, 9H), 1.19 (s, 1H), 1.00 | |
| | | (s, 1H), 0.79 (d, J = 8.7 Hz, 1H), | |
| | | 0.36 (d, J = 13.0 Hz, 1H); ¹⁹F NMR | |
| | | (376 MHz, CD₃OD) | |
| | | δ −61.60, −61.80, −79.49, −81.78. | |
| 53 | ESI-MS m/z calc. | ¹H HMR (400 MHz, CD₃OD) δ | |
| | 505.15485, found | 7.73 (s, 1H), 3.23-3.04 (m, 2H), | |
| | 506.1 (M + 1)⁺; | 2.54 (s, 1H), 2.46 (s, 2H), 2.32 (s, | |
| | Retention time: | 6H), 2.30-2.17 (m, 1H), 2.00- | |
| | 1.4 minutes (LC | 1.77 (m, 2H), 1.67 (d, J = 17.5 Hz, | |
| | Method A) | 1H), 1.16 (m, 1H), 0.86 (s, 1H); ¹⁹F | |
| | | NMR (376 MHz, CD₃OD) | |
| | | δ −61.80, −81.16. | |
| 54 | ESI-MS m/z calc. | ¹H HMR (400 MHz, CD₃OD) δ | |
| | 533.18616, found | 7.73 (s, 1H), 4.41 (m, 1H), 3.18 (dd, | |
| | 534.1 (M + 1)⁺; | J = 9.8, 6.1 Hz, 2H), 2.58-2.31 (m, | |
| | Retention time: | 3H), 2.28-2.11 (m, 6H), 2.05- | |
| | 1.58 minutes (LC | 1.98 (m, 2H), 1.96-1.87 (m, 2H), | |
| | Method A) | 1.84-1.75 (m, 2H), 1.69 (s, 1H), | |
| | | 1.25-1.11 (m, 1H), 0.99-0.81 (m, | |
| | | 1H); ¹⁹F NMR (376 MHz, CD₃OD) | |
| | | δ −61.83, −81.29. | |
| 55 | ESI-MS m/z calc. | ¹H HMR (400 MHz, CD₃OD) δ | |
| | 495.1705, found | 7.74 (s, 1H), 4.11 (q, J = 7.3 Hz, | |
| | 496.1 (M + 1)⁺; | 1H), 3.09 (m, 2H), 2.61-2.37 (m, | |
| | Retention time: | 2H), 2.34-2.16 (m, 1H), 1.92 (m, | |
| | 1.36 minutes (LC | 3H), 1.73 (m, 2H), 1.40 (d, J = 6.9 | |
| | Method A) | Hz, 3H), 1.12 (m, 1H), 0.97 (t, J = | |
| | | 7.4 Hz, 3H), 0.82 (d, J = 26.0 Hz, | |
| | | 1H); ¹⁹F NMR (376 MHz, CD₃OD) | |
| | | δ −61.63, −81.18. | |
| 56 | ESI-MS m/z calc. | ¹H HMR (400 MHz, CD₃OD) δ | |
| | 495.1705, found | 7.74 (s, 1H), 3.99 (q, J = 7.1 Hz, | |
| | 496.1 (M + 1)⁺; | 1H), 3.18-3.04 (m, 2H), 2.59- | |
| | Retention time: | 2.41 (m, 2H), 2.26 (dd, J = 14.3, 8.0 | |
| | 1.36 minutes (LC | Hz, 1H), 2.02 (m, 1H), 1.97-1.81 | |
| | Method A) | (m, 2H), 1.70 (m, J = 14.3, 7.2 Hz, | |
| | | 2H), 1.41 (d, J = 6.9 Hz, 3H), 1.14 | |
| | | (m, 1H), 1.00 (t, J = 7.4 Hz, 3H), | |
| | | 0.83 (d, J = 12.6 Hz, 1H); ¹⁹F NMR | |
| | | (376 MHz, CD₃OD) δ −61.65, −81.23. | |
| 57 | ESI-MS m/z calc. | ¹H HMR (400 MHz, CD₃OD) δ | |
| | 509.18616, found | 7.61 (s, 1H), 3.19-3.05 (m, 2H), | |
| | 510.2 (M + 1)⁺; | 2.84 (t, J = 13.8 Hz, 1H), 2.76- | |
| | Retention time: | 2.61 (m, 1H), 2.53 (t, J = 13.1 Hz, | |
| | 1.48 minutes (LC | 1H), 2.46-2.35 (m, 1H), 2.22- | |
| | Method A) | 2.12 (m, 1H), 2.07-2.00 (m, 1H), | |
| | | 1.81 (s, 2H), 1.64-1.49 (m, 2H), | |
| | | 1.46 (s, 3H), 1.42 (s, 3H), 0.82 (t, J = | |
| | | 7.4 Hz, 3H); ¹⁹F NMR (376 MHz, | |
| | | CD₃OD) δ −61.50, −81.89. | |
| 58 | ESI-MS m/z calc. | ¹H HMR (400 MHz, CD₃OD) δ | |
| | 557.1673, found | 7.74 (s, 1H), 4.18 (t, J = 12.1 Hz, | |
| | 558.1 (M + 1)⁺; | 1H), 3.27-3.02 (m, 2H), 2.66- | |
| | Retention time: | 2.39 (m, 2H), 2.32-2.10 (m, 5H), | |
| | 1.37 minutes (LC | 2.08-1.81 (m, 6H), 1.70 (s, 1H), | |
| | Method A) | 1.20-1.05 (m, 1H), 0.86-0.68 (m, | |
| | | 1H); ¹⁹F NMR (376 MHz, CD₃OD) | |
| | | δ −61.71, −81.07 (d, J = 126.1 | |
| | | Hz), −94.38 (d, J = 238.4 Hz), −103.97 (d, | |
| | | J = 238.0 Hz). | |
| 59 | ESI-MS m/z calc. | ¹H HMR (400 MHz, CD₃OD) δ | |
| | 505.15485, found | 7.73 (s, 1H), 3.18-3.04 (m, 2H), | |
| | 506.1 (M + 1)⁺; | 2.54 (s, 1H), 2.45 (s, 3H), 2.36- | |
| | Retention time: | 2.22 (m, 5H), 2.04-1.76 (m, 2H), | |
| | 1.4 minutes (LC | 1.67 (d, J = 16.5 Hz, 2H), 1.26- | |
| | Method A) | 1.09 (m, 1H), 1.00-0.79 (m, 1H); | |
| | | ¹⁹F NMR (376 MHz, CD₃OD) | |
| | | δ −61.81, −81.20. | |
| 60 | ESI-MS m/z calc. | ¹H HMR (400 MHz, CD₃OD) δ | |
| | 521.18616, found | 7.73 (s, 1H), 4.65-4.49 (m, 1H), | |
| | 522.2 (M + 1)⁺; | 3.21 (dd, J = 9.6, 6.3 Hz, 2H), 2.51- | |
| | Retention time: | 2.35 (m, 2H), 2.30-2.19 (m, 4H), | |
| | 1.51 minutes (LC | 2.17-2.04 (m, 2H), 1.93-1.77 (m, | |
| | Method A) | 2H), 1.70 (s, 1H), 1.26 (s, 3H), 1.19 | |
| | | (s, 3H), 0.99-0.85 (m, 1H); ¹⁹F | |
| | | NMR (376 MHz, CD₃OD) | |
| | | δ −61.82, −81.20. | |
| 61 | ESI-MS m/z calc. | ¹H HMR (400 MHz, CD₃OD) δ | |
| | 493.15485, found | 7.73 (s, 1H), 3.29-3.08 (m, 2H), | |
| | 494.1 (M + 1)⁺; | 2.52-2.38 (m, 2H), 2.27 (m, 1H), | |
| | Retention time: | 2.12-1.99 (m, 1H), 1.93-1.79 (m, | |
| | 1.26 minutes (LC | 1H), 1.75-1.65 (m, 2H), 1.50 (s, | |
| | Method A) | 3H), 1.19 (m, 1H), 1.01-0.76 (m, | |
| | | 4H); ¹⁹F NMR (376 MHz, CD₃OD) | |
| | | δ −61.79, −81.15. | |
| 62 | ESI-MS m/z calc. | ¹H HMR (400 MHz, CD₃OD) δ | |
| | 569.1673, found | 7.73 (s, 1H), 4.53-4.37 (m, 1H), | |
| | 570.1 (M + 1)⁺; | 3.20 (dd, J = 9.3, 6.7 Hz, 2H), 2.72 | |
| | Retention time: | (m, 2H), 2.64-2.50 (m, 6H), 2.49- | |
| | 1.44 minutes (LC | 2.21 (m, 3H), 1.91-1.78 (m, 2H), | |
| | Method A) | 1.69 (s, 1H), 1.22-1.09 (m, 1H), | |
| | | 0.99-0.85 (m, 1H); ¹⁹F NMR (376 | |
| | | MHz, CD₃OD) | |
| | | δ −61.81, −81.15, −92.61 (d, J = 2.2 Hz). | |
| 63 | ESI-MS m/z calc. | ¹H HMR (400 MHz, CD₃OD) δ | |
| | 543.1517, found | 7.62 (s, 1H), 3.18-3.09 (m, 2H), | |
| | 544.1 (M + 1)⁺; | 2.88 (m, 2H), 2.69 (m, 2H), 2.28 | |
| | Retention time: | (dd, J = 14.8, 8.7 Hz, 2H), 2.15 (m, | |
| | 1.38 minutes (LC | 1H), 1.76 (m, 2H), 1.58 (s, 3H), | |
| | Method A) | 1.55-1.47 (m, 1H), 1.10-0.96 (m, | |
| | | 1H), 0.85-0.72 (m, 1H); ¹⁹F NMR | |
| | | (376 MHz, CD₃OD) | |
| | | δ −61.70, −81.13, −85.78 | |
| | | (d, J = 200.0 Hz), −99.76 (d, J = 200.0 Hz). | |
| 64 | ESI-MS m/z calc. | ¹H HMR (400 MHz, DMSO-d₆) δ | |
| | 577.2488, found | 7.78 (s, 1H), 7.71 (s, 1H), 7.20 (s, | |
| | 578.2 (M + 1)⁺; | 2H), 3.95 (q, J = 8.0, 4.7 Hz, 1H), | |
| | Retention time: | 3.07-2.90 (m, 2H), 2.27 (s, 2H), | |
| | 1.24 minutes (LC | 2.17 (t, J = 10.5 Hz, 1H), 1.82 (d, J = | |
| | Method M) | 11.3 Hz, 4H), 1.78-1.64 (m, 4H), | |
| | | 1.57 (s, 1H), 1.10 (dd, J = 24.6, | |
| | | 12.7 Hz, 3H), 0.96 (dd, J = 20.0, | |
| | | 9.1 Hz, 1H), 0.86 (s, 9H), 0.71 (d, J = | |
| | | 6.9 Hz, 1H). | |
| 65 | ESI-MS m/z calc. | ¹H HMR (400 MHz, CD₃OD) δ | |
| | 523.1654, found | 7.75 (s, 1H), 4.33 (m, 1H), 4.12- | |
| | 524.1 (M + 1)⁺; | 3.98 (m, 2H), 3.55 (tdd, J = 11.9, | |
| | Retention time: | 8.2, 2.1 Hz, 2H), 3.25-3.01 (m, | |
| | 1.09 minutes (LC | 2H), 2.48 (s, 2H), 2.33-2.22 (m, | |
| | Method A) | 2H), 2.16 (m, 2H), 1.97-1.82 (m, | |
| | | 2H), 1.79-1.63 (m, 2H), 1.12 (dd, | |
| | | J = 11.7, 5.4 Hz, 1H), 0.79 (s, 1H); | |
| | | ¹⁹F NMR (376 MHz, CD₃OD) | |
| | | δ −61.70, −81.24. | |
| 66 | ESI-MS m/z calc. | ¹H HMR (400 MHz, CD₃OD) δ | |
| | 534.1814, found | 7.75 (s, 1H), 4.25 (q, J = 8.5 Hz, | |
| | 535.2 (M + 1)⁺; | 1H), 4.20 (s, 2H), 4.14 (s, 2H), 3.28- | |
| | Retention time: | 3.10 (m, 2H), 2.84-2.66 (m, 5H), | |
| | 0.71 minutes (LC | 2.51-2.32 (m, 2H), 2.29-2.17 (m, | |
| | Method A) | 1H), 1.89-1.76 (m, 2H), 1.69- | |
| | | 1.62 (m, 1H), 1.20 (m, 1H), 0.94 (s, | |
| | | 1H); ¹⁹F NMR (376 MHz, CD₃OD) | |
| | | δ −61.73, −81.15. One proton | |
| | | obscured by solvent. | |
| 67 | ESI-MS m/z calc. | ¹H HMR (400 MHz, DMSO-d₆) δ | |
| | 507.1705, found | 7.79 (s, 1H), 7.71 (s, 1H), 7.20 (s, | |
| | 508.4 (M + 1)⁺; | 2H), 4.20-4.06 (m, 1H), 3.05 (t, J = | |
| | Retention time: | 7.8 Hz, 2H), 2.34-2.12 (m, 3H), | |
| | 1.79 minutes (LC | 1.91-1.64 (m, 8H), 1.53 (d, J = 8.1 | |
| | Method A) | Hz, 3H), 1.10-0.97 (m, 1H), 0.80 | |
| | | (q, J = 6.9, 6.4 Hz, 1H). | |
| 68 | ESI-MS m/z calc. | ¹H HMR (400 MHz, DMSO-d₆) δ | |
| | 609.12585, found | 7.82 (s, 1H), 7.72 (s, 1H), 7.39 (d, J = | |
| | 610.1 (M + 1)⁺; | 8.3 Hz, 1H), 7.30 (s, 2H), 7.29 (s, | |
| | Retention time: | 1H), 7.17 (dd, J = 8.3, 1.7 Hz, 1H), | |
| | 1.37 minutes (LC | 4.89 (d, J = 15.0 Hz, 1H), 4.53 (d, J = | |
| | Method J) | 15.1 Hz, 1H), 3.20-3.08 (m, 1H), | |
| | | 3.04-2.92 (m, 1H), 2.34-2.22 (m, | |
| | | 2H), 2.22-2.13 (m, 1H), 2.00- | |
| | | 1.87 (m, 1H), 1.75-1.62 (m, 1H), | |
| | | 1.61-1.47 (m, 1H), 1.13-1.00 (m, | |
| | | 1H), 0.92-0.76 (m, 1H). ¹⁹F NMR | |
| | | (376 MHz, DMSO-d₆) δ −49.10 (s, | |
| | | 2F), −59.00 (s, 3F), −78.34 (s, 3F). | |
| 69 | ESI-MS m/z calc. | ¹H HMR (400 MHz, methanol-_{d4}) δ | |
| | 571.1324, found | 7.75 (s, 1H), 4.25 (t, J = 12.3 Hz, | |
| | 572.1 (M + 1)⁺; | 1H), 3.47-3.36 (m, 2H), 3.22- | |
| | Retention time: | 3.10 (m, 4H), 2.81 (p, J = 12.4 Hz, | |
| | 0.99 minutes (LC | 2H), 2.47 (d, J = 16.0 Hz, 2H), 2.34- | |
| | Method A) | 2.12 (m, 3H), 1.97-1.84 (m, 2H), | |
| | | 1.77-1.65 (m, 1H), 1.17 (m, 1H), | |
| | | 0.97-0.78 (m, 1H); ¹⁹F NMR (376 | |
| | | MHz, methanol-d₄) | |
| | | δ −61.67, −81.22. | |
| 70 | ESI-MS m/z calc. | ¹H HMR (400 MHz, methanol-d₄) δ | |
| | 543.10114, found | 7.76 (s, 1H), 4.85-4.78 (m, 1H), | |
| | 544.1 (M + 1)⁺; | 4.68-4.51 (m, 1H), 4.48-4.32 (m, | |
| | Retention time: | 2H), 3.41 (m, 1H), 3.30-3.19 (m, | |
| | 0.96 minutes (LC | 1H), 2.52-2.35 (m, 3H), 2.32- | |
| | Method A) | 2.21 (m, 1H), 1.99-1.89 (m, 1H), | |
| | | 1.84-1.77 (m, 1H), 1.75-1.66 (m, | |
| | | 1H), 1.31-1.19 (m, 1H), 1.06- | |
| | | 0.92 (m, 1H); ¹⁹F NMR (376 MHz, | |
| | | methanol-d₄) δ −61.63, −81.14. | |
| 71 | ESI-MS m/z calc. | | |
| | 561.1611, found | | |
| | 562.2 (M + 1)⁺; | | |
| | Retention time: | | |
| | 1.83 minutes (LC | | |
| | Method A) | | |
| 72 | ESI-MS m/z calc. | ¹H HMR (400 MHz, methanol-d₄) δ | |
| | 561.1611, found | 7.75 (s, 1H), 7.50-7.41 (m, 2H), | |
| | 562.2 (M + 1)⁺; | 7.16-7.05 (m, 2H), 5.83 (q, J = 7.2 | |
| | Retention time: | Hz, 1H), 4.60 (s, 1H), 3.02 (m, 1H), | |
| | 1.84 minutes (LC | 2.96-2.84 (m, 1H), 2.48 (t, J = | |
| | Method A) | 12.6 Hz, 1H), 2.36 (s, 1H), 2.22 (m, | |
| | | 1H), 1.92-1.73 (m, 5H), 1.69- | |
| | | 1.52 (m, 2H), 1.05-0.85 (m, 2H), | |
| | | 0.66 (m, 1H). | |
| 73 | ESI-MS m/z calc. | ¹H HMR (400 MHz, CDCl₃) δ 7.54 | |
| | 613.13715, found | (s, 1H), 7.37-7.29 (m, 2H), 7.21- | |
| | 614.2 (M + 1)⁺; | 7.14 (m, 2H), 6.08 (s, 2H), 5.23 (d, | |
| | Retention time: | J = 15.1 Hz, 1H), 4.48 (s, 1H), 4.37 | |
| | 1.97 minutes (LC | (d, J = 15.1 Hz, 1H), 3.12 (m, J = | |
| | Method A) | 14.6, 11.6, 3.2 Hz, 1H), 2.96 (m, J = | |
| | | 14.0, 10.8, 5.7 Hz, 1H), 2.48 (m, J = | |
| | | 18.5, 9.4, 4.5 Hz, 2H), 2.28 (m, J = | |
| | | 14.3, 7.6, 3.4 Hz, 1H), 1.92-1.73 | |
| | | (m, 1H), 1.64 (m, J = 14.8, 8.1, 4.2 | |
| | | Hz, 1H), 1.30-1.21 (m, 1H), 1.16 | |
| | | (s, 1H), 0.79-0.64 (m, 1H). | |
| 74 | ESI-MS m/z calc. | | |
| | 516.13446, found | | |
| | 517.2 (M + 1)+; | | |
| | Retention time: | | |
| | 1.43 minutes (LC | | |
| | Method A) | | |
| 75 | ESI-MS m/z calc. | | |
| | 515.1392, found | | |
| | 516.3 (M + 1)⁺; | | |
| | Retention time: | | |
| | 1.59 minutes (LC | | |
| | Method A) | | |
| 76 | ESI-MS m/z calc. | | |
| | 519.1454, found | | |
| | 520.3 (M + 1)⁺; | | |
| | Retention time: | | |
| | 1.26 minutes (LC | | |
| | Method A) | | |
| 77 | ESI-MS m/z calc. | | |
| | 579.17053, found | | |
| | 580.1 (M + 1)⁺; | | |
| | Retention time: | | |
| | 1.98 minutes (LC | | |
| | Method A) | | |
| 78 | ESI-MS m/z calc. | | |
| | 579.17053, found | | |
| | 580.2 (M + 1)⁺; | | |
| | Retention time: | | |
| | 1.98 minutes (LC | | |
| | Method A) | | |
| 79 | ESI-MS m/z calc. | ¹H HMR (400 MHz, DMSO-d₆) δ | |
| | 519.1454, found | 7.81 (s, 1H), 7.71 (s, 1H), 7.61 (d, J = | |
| | 520.0 (M + 1)⁺; | 2.1 Hz, 1H), 7.26 (s, 2H), 6.11 (d, | |
| | Retention time: | J = 2.1 Hz, 1H), 4.80 (d, J = 14.7 | |
| | 1.22 minutes (LC | Hz, 1H), 4.47 (d, J = 14.7 Hz, 1H), | |
| | Method A) | 3.20-3.08 (m, 2H), 3.07-2.95 (m, | |
| | | 1H), 2.31-2.11 (m, 3H), 1.98- | |
| | | 1.83 (m, 1H), 1.71-1.60 (m, 1H), | |
| | | 1.59-1.47 (m, 1H), 1.13-1.00 (m, | |
| | | 1H), 0.92-0.75 (m, 1H). ¹⁹F NMR | |
| | | (376 MHz, DMSO-d₆) δ −59.02 (s, | |
| | | 3F), −78.33 (s, 3F). | |
| 80 | ESI-MS m/z calc. | | |
| | 519.1454, found | | |
| | 520.1 (M + 1)⁺; | | |
| | Retention time: | | |
| | 1.19 minutes (LC | | |
| | Method A) | | |
| 81 | ESI-MS m/z calc. | | |
| | 507.1705, found | | |
| | 508.3 (M + 1)⁺; | | |
| | Retention time: | | |
| | 1.63 minutes (LC | | |
| | Method A) | | |
| 82 | ESI-MS m/z calc. | | |
| | 507.1705, found | | |
| | 508.3 (M + 1)⁺; | | |
| | Retention time: | | |
| | 1.78 minutes (LC | | |
| | Method A) | | |
| 83 | ESI-MS m/z calc. | ¹H HMR (400 MHz, Chloroform-d) | |
| | 585.21747, found | 8 7.53 (s, 1H), 7.38-7.32 (m, 2H), | |
| | 586.3 (M + 1)⁺; | 7.24 (t, J = 7.4 Hz, 2H), 5.99 (s, | |
| | Retention time: | 2H), 5.37-5.28 (m, 1H), 4.26 (d, J = | |
| | 2.13 minutes (LC | 14.9 Hz, 1H), 4.04-3.99 (m, 1H), | |
| | Method A) | 3.11-2.91 (m, 2H), 2.51 (m, 2H), | |
| | | 2.26 (m, 1H), 1.88 (m, 1H), 1.77 | |
| | | (m, 1H), 1.30 (s, 9H), 1.17-1.08 | |
| | | (m, 1H), 0.92-0.81 (m, 1H), 0.71- | |
| | | 0.61 (m, 1H). | |
| 84 | ESI-MS m/z calc. | ¹H NMR (400 MHz, DMSO-d₆) δ | |
| | 533.161, found | 7.81 (s, 1H), 7.80-7.60 (bs, 1H), | |
| | 534.1 (M + 1)⁺; | 7.59 (d, J = 2.1 Hz, 1H), 7.25 (s, | |
| | Retention time: | 2H), 6.05 (d, J = 1.9 Hz, 1H), 4.76 | |
| | 1.36 minutes (LC | (d, J = 14.7 Hz, 1H), 4.39 (d, J = | |
| | Method A) | 14.7 Hz, 1H), 3.78 (s, 3H), 3.18- | |
| | | 3.07 (m, 1H), 3.06-2.95 (m, 1H), | |
| | | 2.32-2.12 (m, 3H), 1.98-1.84 (m, | |
| | | 1H), 1.72-1.60 (m, 1H), 1.59- | |
| | | 1.48 (m, 1H), 1.12-1.01 (m, 1H), | |
| | | 0.91-0.77 (m, 1H). ¹⁹F NMR (376 | |
| | | MHz, DMSO-d₆) δ −59.01 (s, | |
| | | 3F), −78.35 (s, 3F). | |
| 85 | ESI-MS m/z calc. | ¹H HMR (400 MHz, DMSO-d₆) δ | |
| | 533.161, found | 7.80 (s, 1H), 7.79-7.62 (bs, 1H), | |
| | 534.1 (M + 1)⁺; | 7.61 (s, 1H), 7.36 (s, 1H), 7.25 (s, | |
| | Retention time: | 2H), 4.60 (d, J = 14.5 Hz, 1H), 4.30 | |
| | 1.32 minutes (LC | (d, J = 14.5 Hz, 1H), 3.79 (s, 3H), | |
| | Method A) | 3.16-3.06 (m, 1H), 3.05-2.95 (m, | |
| | | 1H), 2.30-2.13 (m, 3H), 1.99- | |
| | | 1.86 (m, 1H), 1.72-1.61 (m, 1H), | |
| | | 1.60-1.49 (m, 1H), 1.14-1.02 (m, | |
| | | 1H), 0.93-0.80 (m, 1H). ¹⁹F NMR | |
| | | (376 MHz, DMSO-d₆) δ −58.98 (s, | |
| | | 3F), −78.33 (s, 3F). | |
| 86 | ESI-MS m/z calc. | ¹H HMR (400 MHz, DMSO-d₆) δ | |
| | 586.2127, found | 8.64 (s, 1H), 8.18 (s, 1H), 7.89 (s, | |
| | 587.2 (M + 1)⁺; | 1H), 7.85 (s, 1H), 7.74 (s, 1H), 7.34 | |
| | Retention time: | (s, 2H), 4.93 (d, J = 15.5 Hz, 1H), | |
| | 1.26 minutes (LC | 4.71 (d, J = 15.5 Hz, 1H), 3.33- | |
| | Method A) | 3.22 (m, 1H), 3.16-3.04 (m, 1H), | |
| | | 2.35-2.15 (m, 3H), 2.04-1.92 (m, | |
| | | 1H), 1.74-1.64 (m, 1H), 1.62- | |
| | | 1.51 (m, 1H), 1.42 (s, 9H), 1.16- | |
| | | 1.05 (m, 1H), 0.94-0.84 (m, 1H). | |
| | | ¹⁹F NMR (376 MHz, DMSO-d₆) | |
| | | δ −58.93 (s, 3F), −78.30 (s, 3F). | |
| 87 | ESI-MS m/z calc. | ¹H HMR (400 MHz, DMSO-d₆) δ | |
| | 586.2127, found | 8.71 (s, 1H), 8.36 (s, 1H), 7.84 (s, | |
| | 587.3 (M + 1)⁺; | 1H), 7.74 (s, 1H), 7.55 (d, J = 8.3 | |
| | Retention time: | Hz, 1H), 7.35 (s, 2H), 4.99 (d, J = | |
| | 1.35 minutes (LC | 16.4 Hz, 1H), 4.82 (d, J = 17.3 Hz, | |
| | Method A) | 1H), 3.46-3.35 (m, 1H), 3.29- | |
| | | 3.18 (m, 1H), 2.35-2.14 (m, 3H), | |
| | | 2.02-1.89 (m, 1H), 1.73-1.62 (m, | |
| | | 1H), 1.61-1.50 (m, 1H), 1.35 (s, | |
| | | 9H), 1.18-1.06 (m, 1H), 0.98- | |
| | | 0.86 (m, 1H). ¹⁹F NMR (376 MHz, | |
| | | DMSO-d₆) δ −58.95 (s, 3F), −78.32 | |
| | | (s, 3F). | |
| 88 | ESI-MS m/z calc. | ¹H HMR (400 MHz, CD₃OD) δ | |
| | 523.14545, found | 7.72 (s, 1H), 3.25-3.04 (m, 2H), | |
| | 524.2 (M + 1)⁺; | 2.58 (t, J = 2.0 Hz, 6H), 2.44 (s, | |
| | Retention time: | 2H), 2.25 (t, J = 11.6 Hz, 1H), 1.97- | |
| | 1.44 minutes (LC | 1.77 (m, 2H), 1.68 (s, 1H), 1.19- | |
| | Method A) | 1.03 (m, 1H), 0.95-0.77 (m, 1H); | |
| | | ¹⁹F NMR (376 MHz, CD₃OD) | |
| | | δ −61.83, −81.05 (d, J = 97.5 | |
| | | Hz), −170.64 | |
| 89 | ESI-MS m/z calc. | ¹H HMR (400 MHz, CD₃OD) δ | |
| | 519.17053, found | 7.73 (s, 1H), 3.12 (m, 2H), 2.45 (s, | |
| | 520.2 (M + 1)⁺; | 2H), 2.35-2.22 (m, 1H), 2.16 (s, | |
| | Retention time: | 6H), 1.86 (m, 1H), 1.69 (s, 2H), | |
| | 1.53 minutes (LC | 1.30 (s, 3H), 1.15 (m, 1H), 0.92- | |
| | Method A) | 0.77 (m, 1H); ¹⁹F NMR (376 MHz, | |
| | | CD₃OD) δ −61.80, −81.25. | |
| 90 | ESI-MS m/z calc. | ¹H HMR (400 MHz, CD₃OD) δ | |
| | 561.21747, found | 7.73 (s, 1H), 3.14 (m, 2H), 2.45 (s, | |
| | 562.3 (M + 1)⁺; | 2H), 2.34-2.22 (m, 1H), 2.11 (t, J = | |
| | Retention time: | 9.6 Hz, 6H), 1.98-1.79 (m, 1H), | |
| | 1.77 minutes (LC | 1.75-1.63 (m, 2H), 1.21-1.10 (m, | |
| | Method A) | 1H), 0.96 (s, 9H), 0.92-0.81 (m, | |
| | | 1H); ¹⁹F NMR (376 MHz, CD₃OD) | |
| | | δ −61.79, −81.16. | |
| 91 | ESI-MS m/z calc. | ¹H HMR (400 MHz, DMSO-d₆) δ | |
| | 651.1728, found | 7.80 (s, 1H), 7.75 (s, 1H), 7.39 (d, J = | |
| | 652.32 (M + 1)⁺; | 8.3 Hz, 1H), 7.32 (s, 1H), 7.25- | |
| | Retention time: | 7.16 (m, 3H), 4.78 (q, 2H), 3.07 (d, | |
| | 2.08 minutes (LC | J = 14.7 Hz, 1H), 2.96 (d, 1H), 2.27- | |
| | Method A) | 2.17 (m, 2H), 1.86-1.80 (m, 1H), | |
| | | 1.55-1.45 (m, 1H), 1.25-1.23 (m, | |
| | | 1H), 1.08-1.03 (m, 1H), 0.95- | |
| | | 0.89 (m, 1H), 0.87-0.85 (m, 1H), | |
| | | 0.79 (s, 3H), 0.69 (s, 3H). | |
| 92 | ESI-MS m/z calc. | ¹H HMR (400 MHz, CD₃OD) δ | |
| | 533.18616, found | 7.61 (s, 1H), 3.14 (d, J = 10.5 Hz, | |
| | 534.2 (M + 1)⁺; | 1H), 3.07-2.89 (m, 1H), 2.54 (s, | |
| | Retention time: | 1H), 2.43 (d, J = 10.4 Hz, 2H), 2.19- | |
| | 1.67 minutes (LC | 2.08 (m, 1H), 1.89-1.47 (m, 9H), | |
| | Method A) | 1.43-1.31 (m, 2H), 1.20 (s, 2H), | |
| | | 1.05 (d, J = 17.2 Hz, 1H), 0.80 (dd, | |
| | | J = 7.4, 4.0 Hz, 1H). ¹⁹F NMR (376 | |
| | | MHz, CD³OD) δ −61.78, −81.85. | |
| 93 | ESI-MS m/z calc. | | |
| | 516.13446, found | | |
| | 517.2 (M + 1)⁺; | | |
| | Retention time: | | |
| | 1.08 minutes (LC | | |
| | Method A) | | |
| 94 | ESI-MS m/z calc. | | |
| | 543.17053, found | | |
| | 544.3 (M + 1)⁺; | | |
| | Retention time: | | |
| | 1.87 minutes (LC | | |
| | Method A) | | |
| 95 | ESI-MS m/z calc. | ¹H HMR (400 MHz, CD₃OD) δ | |
| | 561.1923, found | 7.94 (s, 1H), 7.79 (s, 1H), 7.74 (s, | |
| | 562.3 (M + 1)⁺; | 1H), 4.77 (d, J = 14.8 Hz, 1H), 4.61 | |
| | Retention time: | (m, 1H), 4.45 (d, J = 14.8 Hz, 1H), | |
| | 1.5 minutes (LC | 3.32-3.21 (m, 1H), 3.15 (m, 1H), | |
| | Method A) | 2.44 (m, 2H), 2.24 (m, 1H), 2.01 | |
| | | (m, 1H), 1.80 (m, 1H), 1.78-1.61 | |
| | | (m, 1H), 1.52 (d, J = 6.7 Hz, 6H), | |
| | | 1.31-1.17 (m, 1H), 0.95 (m, 1H). | |
| 96 | ESI-MS m/z calc. | ¹H HMR (400 MHz, CD₃OD) δ | |
| | 561.1923, found | 7.76 (s, 1H), 7.67 (d, J = 2.1 Hz, | |
| | 562.3 (M + 1)⁺; | 1H), 6.39 (d, J = 2.1 Hz, 1H), 5.10 | |
| | Retention time: | (d, J = 15.8 Hz, 1H), 4.84-4.74 (m, | |
| | 1.55 minutes (LC | 2H), 3.35 (s, 1H), 3.10 (m, 1H), | |
| | Method A) | 2.45 (m, 2H), 2.24 (m, 1H), 1.94 | |
| | | (m, 1H), 1.82 (m, 1H), 1.66 (q, J = | |
| | | 8.7, 7.1 Hz, 1H), 1.50 (dd, J = 11.0, | |
| | | 6.6 Hz, 6H), 1.17 (m, 1H), 0.94- | |
| | | 0.85 (m, 1H). | |
| 97 | ESI-MS m/z calc. | ¹H HMR (400 MHz, DMSO-d₆) δ | |
| | 537.1811, found | 7.86-7.76 (m, 1H), 7.77-7.68 (m, | |
| | 538.1 (M + 1)⁺; | 1H), 7.23 (s, 2H), 3.92-3.80 (m, | |
| | Retention time: | 1H), 3.70-3.53 (m, 2H), 3.30- | |
| | 1.74 minutes (LC | 3.05 (m, 4H), 2.40-2.05 (m, 3H), | |
| | Method A) | 1.88-1.74 (m, 2H), 1.72-1.61 (m, | |
| | | 1H), 1.61-1.52 (m, 2H), 1.52- | |
| | | 1.39 (m, 3H), 1.28-1.17 (m, 1H), | |
| | | 1.15-1.04 (m, 1H), 0.84-0.70 (m, 1H). | |
| 98 | ESI-MS m/z calc. | ¹H HMR (400 MHz, DMSO-d₆) δ | |
| | 537.1811, found | 7.80 (s, 1H), 7.78-7.71 (m, 1H), | |
| | 538.1 (M + 1)⁺; | 7.33-7.07 (m, 2H), 3.78-3.65 (m, | |
| | Retention time: | 2H), 3.52-3.42 (m, 1H), 3.31- | |
| | 1.6 minutes (LC | 3.25 (m, 1H), 3.25-3.05 (m, 4H), | |
| | Method A) | 2.33-2.12 (m, 3H), 2.06-1.97 (m, | |
| | | 1H), 1.93-1.82 (m, 1H), 1.77- | |
| | | 1.65 (m, 2H), 1.65-1.50 (m, 2H), | |
| | | 1.50-1.39 (m, 1H), 1.30-1.19 (m, | |
| | | 1H), 1.12-0.99 (m, 1H), 0.89- | |
| | | 0.77 (m, 1H). | |
| 99 | ESI-MS m/z calc. | ¹H HMR (400 MHz, DMSO-d₆) δ | |
| | 537.1811, found | 7.80 (s, 1H), 7.71 (s, 1H), 7.22 (s, | |
| | 538.1 (M + 1)⁺; | 2H), 3.90-3.79 (m, 2H), 3.48 (dd, | |
| | Retention time: | J = 13.3, 7.6 Hz, 1H), 3.30-3.21 | |
| | 1.52 minutes (LC | (m, 2H), 3.21-2.98 (m, 3H), 2.31- | |
| | Method A) | 2.12 (m, 3H), 2.10-1.97 (m, 1H), | |
| | | 1.95-1.82 (m, 1H), 1.76-1.63 (m, | |
| | | 1H), 1.62-1.54 (m, 1H), 1.54- | |
| | | 1.45 (m, 2H), 1.31-1.17 (m, 2H), | |
| | | 1.13-1.01 (m, 1H), 0.92-0.77 (m, | |
| | | 1H); ¹⁹F NMR (376 MHz, DMSO- | |
| | | d₆) δ −59.22 (s, 3F), −78.32 (s, 3F). | |
| 100 | ESI-MS m/z calc. | ¹H HMR (400 MHz, DMSO-d₆) δ | |
| | 523.1654, found | 7.79 (s, 1H), 7.74 (s, 1H), 7.24 (s, | |
| | 524.1 (M + 1)⁺; | 2H), 4.08-3.93 (m, 1H), 3.85- | |
| | Retention time: | 3.74 (m, 2H), 3.65-3.55 (m, 1H), | |
| | 1.54 minutes (LC | 3.31-3.22 (m, 1H), 3.07-3.01 (m, | |
| | Method A) | 1H), 2.36-2.11 (m, 4H), 2.11- | |
| | | 1.95 (m, 1H), 1.87-1.53 (m, 6H), | |
| | | 1.08-0.92 (m, 1H), 0.78-0.58 (m, 1H). | |
| 101 | ESI-MS m/z calc. | ¹H HMR (400 MHz, DMSO-d₆) δ | |
| | 543.17053, found | 7.82 (s, 1H), 7.68 (s, 1H), 7.25 (s, | |
| | 544.3 (M + 1)⁺; | 2H), 7.21-7.11 (m, 4H), 4.86 (d, J = | |
| | Retention time: | 14.9 Hz, 1H), 4.46 (d, J = 15.0 | |
| | 1.92 minutes (LC | Hz, 1H), 3.16-3.02 (m, 1H), 3.00- | |
| | Method A) | 2.87 (m, 1H), 2.28 (s, 3H), 2.27- | |
| | | 2.12 (m, 3H), 1.96-1.83 (m, 1H), | |
| | | 1.74-1.60 (m, 1H), 1.60-1.46 (m, | |
| | | 1H), 1.09-0.98 (m, 1H), 0.87- | |
| | | 0.79 (m, 1H). | |
| 102 | ESI-MS m/z calc. | ¹H HMR (400 MHz, DMSO-d₆) δ | |
| | 597.1423, found | 7.83 (s, 1H), 7.72 (d, J = 8.1 Hz, | |
| | 598.2 (M + 1)⁺; | 2H), 7.69 (s, 1H), 7.51 (d, J = 8.0 | |
| | Retention time: | Hz, 2H), 7.28 (s, 2H), 4.94 (d, J = | |
| | 1.97 minutes (LC | 15.6 Hz, 1H), 4.65 (d, J = 15.6 Hz, | |
| | Method A) | 1H), 3.27-3.16 (m, 1H), 3.09- | |
| | | 2.98 (m, 1H), 2.31-2.14 (m, 3H), | |
| | | 1.99-1.87 (m, 1H), 1.73-1.60 (m, | |
| | | 1H), 1.60-1.46 (m, 1H), 1.15- | |
| | | 0.99 (m, 1H), 0.90-0.80 (m, 1H). | |
| 103 | ESI-MS m/z calc. | ¹H HMR (400 MHz, DMSO-d₆) δ | |
| | 547.14545, found | 7.82 (s, 1H), 7.69 (s, 1H), 7.38- | |
| | 548.2 (M + 1)⁺; | 7.31 (m, 2H), 7.26 (s, 2H), 7.21- | |
| | Retention time: | 7.12 (m, 2H), 4.86 (d, J = 15.0 Hz, | |
| | 1.82 minutes (LC | 1H), 4.51 (d, J = 14.9 Hz, 1H), 3.19- | |
| | Method A) | 3.05 (m, 1H), 3.04-2.91 (m, 1H), | |
| | | 2.29-2.13 (m, 3H), 1.98-1.83 (m, | |
| | | 1H), 1.66 (s, 1H), 1.55 (s, 1H), 1.13- | |
| | | 0.97 (m, 1H), 0.87-0.79 (m, 1H). | |
| 104 | ESI-MS m/z calc. | ¹H HMR (400 MHz, DMSO-d₆) δ | |
| | 543.17053, found | 7.81 (s, 1H), 7.67 (s, 1H), 7.34- | |
| | 544.2 (M + 1)⁺; | 7.16 (m, 7H), 3.74-3.62 (m, 1H), | |
| | Retention time: | 3.58-3.47 (m, 1H), 3.18-3.06 (m, | |
| | 1.87 minutes (LC | 1H), 3.04-2.92 (m, 1H), 2.89 (t, J = | |
| | Method A) | 7.6 Hz, 2H), 2.31-2.09 (m, 3H), | |
| | | 1.92-1.77 (m, 1H), 1.71-1.60 (m, | |
| | | 1H), 1.60-1.47 (m, 1H), 1.13- | |
| | | 0.98 (m, 1H), 0.90-0.78 (m, 1H). | |
| 105 | ESI-MS m/z calc. | ¹H HMR (400 MHz, DMSO-d₆) δ | |
| | 559.1654, found | 7.83 (s, 1H), 7.69 (s, 1H), 7.29- | |
| | 560.2 (M + 1)+; | 7.21 (m, 3H), 6.90-6.79 (m, 3H), | |
| | Retention time: | 4.88 (d, J = 15.1 Hz, 1H), 4.50 (d, J = | |
| | 1.78 minutes (LC | 15.1 Hz, 1H), 3.72 (s, 3H), 3.17- | |
| | Method A) | 3.07 (m, 1H), 3.03-2.91 (m, 1H), | |
| | | 2.34-2.24 (m, 2H), 2.23-2.12 (m, | |
| | | 1H), 1.96-1.92 (m, 1H), 1.72- | |
| | | 1.64 (m, 1H), 1.57-1.46 (m, 1H), | |
| | | 1.07-0.99 (m, 1H), 0.89-0.80 (m, 1H). | |
| 106 | ESI-MS m/z calc. | ¹H HMR (400 MHz, DMSO-d₆) δ | |
| | 571.2018, found | 7.83 (s, 1H), 7.74 (s, 1H), 7.34- | |
| | 572.3 (M + 1)⁺; | 7.07 (m, 6H), 4.88 (d, J = 15.1 Hz, | |
| | Retention time: | 1H), 4.51 (d, J = 15.2 Hz, 1H), 3.19- | |
| | 2.07 minutes (LC | 3.05 (m, 1H), 2.99-2.90 (m, | |
| | Method A) | 1H), 2.90-2.77 (m, 1H), 2.35- | |
| | | 2.21 (m, 2H), 2.20-2.12 (m, 1H), | |
| | | 1.95-1.81 (m, 1H), 1.74-1.61 (m, | |
| | | 1H), 1.59-1.47 (m, 1H), 1.18 (d, J = | |
| | | 6.9, 1.9 Hz, 6H), 1.07-1.00 (m, | |
| | | 1H), 0.85-0.75 (m, 1H). | |
| 107 | ESI-MS m/z calc. | ¹H HMR (400 MHz, methanol-d₄) δ | |
| | 760.28076, found | 8.62-8.55 (m, 1H), 8.47 (d, J = 7.9 | |
| | 761.4 (M + 1)⁺; | Hz, 1H), 7.92 (dd, J = 7.9, 5.9 Hz, | |
| | Retention time: | 1H), 7.75 (s, 1H), 5.10 (d, J = 16.3 | |
| | 1.01 minutes (LC | Hz, 1H), 4.93 (d, J = 16.4 Hz, 1H), | |
| | Method A) | 3.81-3.67 (m, 1H), 3.46 (m, 1H), | |
| | | 2.62 (s, 3H), 2.51 (m, 1H), 2.48- | |
| | | 2.41 (m, 1H), 2.34-2.21 (m, 1H), | |
| | | 2.07 (q, J = 6.1, 5.5 Hz, 1H), 1.85- | |
| | | 1.66 (m, 2H), 1.35 (m, 1H), 1.21- | |
| | | 1.08 (m, 1H). | |
| 108 | ESI-MS m/z calc. | ¹H HMR (400 MHz, CD₃OD) δ | |
| | 613.13715, found | 7.77 (s, 1H), 7.42 (m, 2H), 7.37 (dd, | |
| | 614.3 (M + 1)⁺; | J = 11.7, 2.7 Hz, 2H), 7.36-7.30 | |
| | Retention time: | (m, 1H), 5.05 (d, J = 15.8 Hz, 1H), | |
| | 2.0 minutes (LC | 4.74 (d, J = 15.8 Hz, 1H), 3.07 (m, | |
| | Method A) | 1H), 2.45 (s, 2H), 2.50-2.39 (m, | |
| | | 1H), 2.30-2.16 (m, 1H), 1.97- | |
| | | 1.90 (m, 1H), 1.80 (m, 1H), 1.66 | |
| | | (m, 1H), 1.16 (m, 1H), 0.90 (m, 1H). | |
| 109 | ESI-MS m/z calc. | ¹H HMR (400 MHz, CD₃OD) δ | |
| | 613.13715, found | 7.76 (s, 1H), 7.44 (t, J = 7.9 Hz, | |
| | 614.2 (M + 1)⁺; | 1H), 7.35 (d, J = 7.7 Hz, 1H), 7.28 | |
| | Retention time: | (s, 1H), 7.19 (d, J = 8.2 Hz, 1H), | |
| | 1.96 minutes (LC | 5.03 (d, J = 15.4 Hz, 1H), 4.67 (d, J = | |
| | Method A) | 15.4 Hz, 1H), 3.25 (dd, J = 11.6, | |
| | | 3.5 Hz, 1H), 3.14-3.02 (m, 1H), | |
| | | 2.44 (m, 2H), 2.23 (m, 1H), 1.94 | |
| | | (m, 1H), 1.86-1.74 (m, 1H), 1.66 | |
| | | (s, 1H), 1.17 (s, 1H), 0.87 (m, 1H). | |
| 110 | ESI-MS m/z calc. | ¹H HMR (400 MHz, DMSO-d₆) δ | |
| | 587.1967, found | 7.81 (s, 1H), 7.71 (s, 1H), 7.26 (s, | |
| | 588.3 (M + 1)⁺; | 2H), 7.21 (d, J = 8.3 Hz, 2H), 6.89- | |
| | Retention time: | 6.84 (m, 2H), 4.83 (d, J = 14.7 Hz, | |
| | 1.94 minutes (LC | 1H), 4.62-4.54 (m, 1H), 4.42 (d, J = | |
| | Method A) | 14.8 Hz, 1H), 3.12-3.02 (m, 1H), | |
| | | 2.99-2.87 (m, 1H), 2.29-2.22 (m, | |
| | | 2H), 2.22-2.14 (m, 1H), 1.96- | |
| | | 1.84 (m, 1H), 1.72-1.62 (m, 1H), | |
| | | 1.59-1.48 (m, 1H), 1.24 (d, J = 5.9 | |
| | | Hz, 6H), 1.09-0.99 (m, 1H), 0.86- | |
| | | 0.77 (m, 1H). | |
| 111 | ESI-MS m/z calc. | ¹H HMR (400 MHz, CD₃OD) δ | |
| | 563.1159, found | 7.66 (s, 1H), 7.23 (s, 4H), 4.92 (d, J = | |
| | 564.08 (M + 1)⁺; | 15.2 Hz, 1H), 4.45 (d, J = 15.1 | |
| | Retention time: | Hz, 1H), 3.13 (m, 1H), 2.95 (m, | |
| | 1.29 minutes (LC | 1H), 2.34 (m, 2H), 2.14 (m, 1H), | |
| | Method A) | 1.92-1.78 (m, 1H), 1.69 (m, 1H), | |
| | | 1.57 (dd, J = 12.7, 7.6 Hz, 1H), | |
| | | 1.05 (m, 1H), 0.87-0.71 (m, 1H). | |
| 112 | ESI-MS m/z calc. | ¹H HMR (400 MHz, DMSO-d₆) δ | |
| | 565.136, found | 7.82 (s, 1H), 7.71 (s, 1H), 7.37- | |
| | 566.3 (M + 1)⁺; | 7.22 (m, 4H), 7.14-7.06 (m, 1H), | |
| | Retention time: | 4.84 (d, J = 15.3 Hz, 1H), 4.58 (d, J = | |
| | 1.86 minutes (LC | 15.3 Hz, 1H), 3.25-3.15 (m, 1H), | |
| | Method A) | 3.08-2.98 (m, 1H), 2.31-2.22 (m, | |
| | | 2H), 2.22-2.13 (m, 1H), 1.94- | |
| | | 1.85 (m, 1H), 1.72-1.62 (m, 1H), | |
| | | 1.59-1.50 (m, 1H), 1.11-1.02 (m, | |
| | | 1H), 0.89-0.82 (m, 1H). | |
| 113 | ESI-MS m/z calc. | ¹H HMR (400 MHz, DMSO-d₆) δ | |
| | 565.136, found | 7.82 (s, 1H), 7.71 (s, 1H), 7.47- | |
| | 566.2 (M + 1)⁺; | 7.38 (m, 1H), 7.36-7.24 (m, 3H), | |
| | Retention time: | 7.20-7.14 (m, 1H), 4.85 (d, J = | |
| | 1.84 minutes (LC | 15.2 Hz, 1H), 4.51 (d, J = 15.2 Hz, | |
| | Method A) | 1H), 3.20-3.11 (m, 1H), 3.04- | |
| | | 2.95 (m, 1H), 2.35-2.23 (m, 2H), | |
| | | 2.23-2.14 (m, 1H), 1.97-1.87 (m, | |
| | | 1H), 1.73-1.63 (m, 1H), 1.59- | |
| | | 1.50 (m, 1H), 1.11-1.01 (m, 1H), | |
| | | 0.87-0.81 (m, 1H). | |
| 114 | ESI-MS m/z calc. | ¹H HMR (400 MHz, methanol-d₄) δ | |
| | 598.1375, found | 7.76 (s, 1H), 7.31 (d, J = 2.1 Hz, | |
| | 599.1 (M + 1)⁺; | 1H), 7.22 (dd, J = 11.3, 1.9 Hz, | |
| | Retention time: | 2H), 5.02 (d, J = 15.2 Hz, 1H), 4.55 | |
| | 1.76 minutes (LC | (d, J = 15.2 Hz, 1H), 3.28-3.17 | |
| | Method A) | (m, 1H), 3.11-2.99 (m, 1H), 2.51- | |
| | | 2.39 (m, 2H), 2.29 (s, 3H), 2.27- | |
| | | 2.19 (m, 1H), 2.02-1.89 (m, 1H), | |
| | | 1.88-1.73 (m, 1H), 1.73-1.60 (m, | |
| | | 1H), 1.21-1.11 (m, 1H), 0.88 (s, 1H). | |
| 115 | ESI-MS m/z calc. | ¹H HMR (400 MHz, CD₃OD) δ | |
| | 571.2018, found | 7.78 (s, 1H), 7.35-7.01 (m, 4H), | |
| | 572.19 (M + 1)⁺; | 5.05 (d, J = 15.0 Hz, 1H), 4.59 (d, J = | |
| | Retention time: | 15.0 Hz, 1H), 3.20 (m, 1H), 3.05 | |
| | 2.04 minutes (LC | (m, 1H), 2.59 (t, J = 7.6 Hz, 2H), | |
| | Method A) | 2.52-2.41 (m, 2H), 2.24 (m, 1H), | |
| | | 2.04-1.86 (m, 1H), 1.81 (m, 1H), | |
| | | 1.65 (m, 3H), 1.13 (m, 1H), 0.94 (t, | |
| | | J = 7.3 Hz, 3H), 0.84 (m, 1H). | |
| 116 | ESI-MS m/z calc. | ¹H NMR (400 MHz, CD₃OD) δ | |
| | 563.1159, found | 7.76 (s, 1H), 7.46-7.21 (m, 4H), | |
| | 564.12 (M + 1)⁺; | 5.03 (d, J = 15.3 Hz, 1H), 4.57 (d, J = | |
| | Retention time: | 15.3 Hz, 1H), 3.24 (m, 1H), 3.06 | |
| | 1.87 minutes (LC | (m, 1H), 2.45 (m, 2H), 2.24 (m, | |
| | Method A) | 1H), 1.97 (m, 1H), 1.79 (m, 1H), | |
| | | 1.65 (m, 1H), 1.16 (m, 1H), 0.87 | |
| | | (m, 1H). | |
| 117 | ESI-MS m/z calc. | ¹H HMR (400 MHz, DMSO-d₆) δ | |
| | 599.1967, found | 7.81 (s, 1H), 7.73 (s, 1H), 7.26 (s, | |
| | 600.3 (M + 1)⁺; | 2H), 7.12 (s, 1H), 7.03 (d, J = 8.1 | |
| | Retention time: | Hz, 1H), 6.64 (d, J = 8.2 Hz, 1H), | |
| | 1.93 minutes (LC | 4.82 (d, J = 14.6 Hz, 1H), 4.39 (d, J = | |
| | Method A) | 14.7 Hz, 1H), 3.10-3.01 (m, | |
| | | 1H), 2.99-2.88 (m, 3H), 2.35- | |
| | | 2.22 (m, 2H), 2.22-2.13 (m, 1H), | |
| | | 1.97-1.83 (m, 1H), 1.74-1.63 (m, | |
| | | 1H), 1.58-1.49 (m, 1H), 1.44- | |
| | | 1.35 (m, 6H), 1.10-0.96 (m, 1H), | |
| | | 0.85-0.73 (m, 1H). | |
| 118 | ESI-MS m/z calc. | ¹H HMR (400 MHz, DMSO-d₆) δ | |
| | 637.1735, found | 7.82 (s, 1H), 7.73 (s, 1H), 7.42 (d, | |
| | 638.3 (M + 1)⁺; | 2H), 7.30 (d, J = 8.0 Hz, 3H), 4.89 | |
| | Retention time: | (d, J = 15.3 Hz, 1H), 4.54 (d, J = | |
| | 2.05 minutes (LC | 15.4 Hz, 1H), 3.21-3.07 (m, 1H), | |
| | Method A) | 2.99-2.91 (m, 1H), 2.34-2.23 (m, | |
| | | 2H), 2.22-2.11 (m, 1H), 1.98- | |
| | | 1.85 (m, 1H), 1.72-1.63 (m, 1H), | |
| | | 1.57-1.46 (m, 1H), 1.36-1.28 (m, | |
| | | 2H), 1.14-1.08 (m, 2H), 1.06- | |
| | | 0.98 (m, 1H), 0.86-0.75 (m, 1H). | |
| 119 | ESI-MS m/z calc. | ¹H HMR (400 MHz, DMSO-d₆) δ | |
| | 569.18616, found | 7.80 (s, 1H), 7.73 (s, 1H), 7.38- | |
| | 570.2 (M + 1)⁺; | 7.14 (m, 6H), 4.48-4.35 (m, 1H), | |
| | Retention time: | 3.26-3.09 (m, 3H), 2.74-2.62 (m, | |
| | 1.98 minutes (LC | 2H), 2.38-2.09 (m, 5H), 1.86- | |
| | Method A) | 1.75 (m, 1H), 1.75-1.64 (m, 1H), | |
| | | 1.64-1.51 (m, 1H), 1.13-1.01 (m, | |
| | | 1H), 0.90-0.79 (m, 1H). | |
| 120 | ESI-MS m/z calc. | ¹H HMR (400 MHz, CD₃OD) δ | |
| | 647.0982, found | 7.77 (s, 1H), 7.56 (d, J = 1.7 Hz, | |
| | 648.2 (M + 1)⁺; | 1H), 7.40 (s, 2H), 7.44-7.35 (m, | |
| | Retention time: | 1H), 5.02 (d, J = 15.5 Hz, 1H), 4.61 | |
| | 2.08 minutes (LC | (d, J = 15.4 Hz, 1H), 3.31-3.23 (m, | |
| | Method A) | 1H), 3.09 (m, 1H), 2.45 (m, 2H), | |
| | | 2.27-2.19 (m, 1H), 1.97 (m, 1H), | |
| | | 1.80 (m, 1H), 1.67 (m, 1H), 1.18 | |
| | | (m, 1H), 0.91 (m, 1H). | |
| 121 | ESI-MS m/z calc. | ¹H HMR (400 MHz, CD₃OD) δ | |
| | 627.15283, found | 7.74 (s, 1H), 7.60-7.53 (m, 2H), | |
| | 628.3 (M + 1)⁺; | 7.29 (d, J = 8.3 Hz, 2H), 5.75 (q, J = | |
| | Retention time: | 7.1 Hz, 1H), 3.18 (m, 1H), 3.01 | |
| | 2.02 minutes (LC | (m, 1H), 2.30-2.17 (m, 2H), 2.17- | |
| | Method A) | 2.07 (m, 1H), 1.79 (s, 1H), 1.74 (d, | |
| | | J = 7.1 Hz, 3H), 1.44 (s, 1H), 1.15 | |
| | | (s, 1H), 0.99-0.88 (m, 1H), 0.62 (s, 1H). | |
| 122 | ESI-MS m/z calc. | ¹H HMR (400 MHz, CD₃OD) δ | |
| | 559.1654, found | 7.75 (s, 1H), 7.26 (m, 1H), 7.22 (dd, | |
| | 560.2 (M + 1)⁺; | J = 7.6, 1.6 Hz, 1H), 6.99 (d, J = | |
| | Retention time: | 8.2 Hz, 1H), 6.91 (t, J = 7.5 Hz, | |
| | 1.8 minutes (LC | 1H), 4.97 (d, J = 15.5 Hz, 1H), 4.67 | |
| | Method A) | (d, J = 15.5 Hz, 1H), 3.87 (s, 3H), | |
| | | 3.35 (s, 1H), 3.22 (m, 1H), 3.05 (m, | |
| | | 1H), 2.45 (m, 1H), 2.44 (s, 2H), | |
| | | 2.29-2.18 (m, 1H), 1.94 (m, 1H), | |
| | | 1.78 (m, 1H), 1.65 (s, 1H), 1.14 (m, | |
| | | 1H), 0.88 (m, 1H). | |
| 123 | ESI-MS m/z calc. | ¹H HMR (400 MHz, CD₃OD) δ | |
| | 597.1423, found | 7.79 (s, 1H), 7.73 (d, J = 7.9 Hz, | |
| | 598.3 (M + 1)⁺; | 1H), 7.59 (t, J = 7.6 Hz, 1H), 7.46 | |
| | Retention time: | (d, J = 7.3 Hz, 2H), 5.16 (d, J = | |
| | 1.97 minutes (LC | 16.4 Hz, 1H), 4.82 (s, 1H), 3.35 (s, | |
| | Method A) | 1H), 3.09 (m, 1H), 2.55-2.39 (m, | |
| | | 2H), 2.24 (m, 1H), 2.00-1.85 (m, | |
| | | 1H), 1.80 (m, 1H), 1.64 (dd, J = | |
| | | 13.6, 5.4 Hz, 1H), 1.16 (m, 1H), | |
| | | 0.90 (m, 1H). | |
| 124 | ESI-MS m/z calc. | ¹H HMR (400 MHz, CD₃OD) δ | |
| | 627.15283, found | 7.77 (s, 1H), 7.28 (d, J = 8.4 Hz, | |
| | 628.3 (M + 1)⁺; | 1H), 7.13 (d, J = 2.6 Hz, 1H), 7.10- | |
| | Retention time: | 7.03 (m, 1H), 5.00 (d, J = 15.7 Hz, | |
| | 2.06 minutes (LC | 1H), 4.65 (d, J = 15.7 Hz, 1H), 3.30- | |
| | Method A) | 3.22 (m, 1H), 3.05 (m, 1H), 2.51- | |
| | | 2.40 (m, 2H), 2.39 (s, 3H), 2.24 (m, | |
| | | 1H), 1.94 (dd, J = 11.1, 5.5 Hz, | |
| | | 1H), 1.80 (m, 1H), 1.65 (t, J = 11.2 | |
| | | Hz, 1H), 1.13 (m, 1H), 1.13 (s, 1H), | |
| | | 0.92-0.82 (m, 1H). | |
| 125 | ESI-MS m/z calc. | ¹H HMR (400 MHz, CD₃OD) δ | |
| | 643.1477, found | 7.75 (s, 1H), 7.28 (d, J = 8.4 Hz, | |
| | 644.3 (M + 1)⁺; | 1H), 6.90 (d, J = 2.2 Hz, 1H), 6.87- | |
| | Retention time: | 6.80 (m, 1H), 4.93 (d, J = 15.7 Hz, | |
| | 2.01 minutes (LC | 1H), 4.65 (d, J = 15.7 Hz, 1H), 3.89 | |
| | Method A) | (s, 3H), 3.35 (s, 1H), 3.30-3.22 (m, | |
| | | 1H), 3.08 (m, 1H), 2.50-2.39 (m, | |
| | | 2H), 2.24 (m, 1H), 1.95 (d, J = 11.4 | |
| | | Hz, 1H), 1.78 (m, 1H), 1.16 (m, | |
| | | 1H), 0.95-0.86 (m, 1H). | |
| 126 | ESI-MS m/z calc. | ¹H NMR (400 MHz, CD₃OD) δ | |
| | 631.12775, found | 7.75 (s, 1H), 7.48 (t, J = 8.4 Hz, | |
| | 632.2 (M + 1)⁺; | 1H), 7.18-7.08 (m, 2H), 5.01 (d, J = | |
| | Retention time: | 15.6 Hz, 1H), 4.71 (d, J = 15.6 | |
| | 2.03 minutes (LC | Hz, 1H), 3.35 (s, 1H), 3.12 (m, 1H), | |
| | Method A) | 2.45 (m, 2H), 2.24 (m, 1H), 1.97 | |
| | | (m, 1H), 1.80 (m, 1H), 1.71-1.62 | |
| | | (m, 1H), 1.19 (m, 1H), 0.91 (m, 1H). | |
| 127 | ESI-MS m/z calc. | ¹H HMR (400 MHz, methanol-d₄) δ | |
| | 577.156, found | 7.75 (s, 1H), 7.16-6.97 (m, 3H), | |
| | 578.2 (M + 1)+; | 4.99 (d, J = 14.9 Hz, 1H), 4.47 (d, J = | |
| | Retention time: | 14.9 Hz, 1H), 3.31 (s, 3H), 3.26- | |
| | 1.74 minutes (LC | 3.14 (m, 1H), 3.11-2.99 (m, 1H), | |
| | Method A) | 2.53-2.35 (m, 2H), 2.29-2.19 (m, | |
| | | 1H), 2.03-1.89 (m, 1H), 1.86- | |
| | | 1.74 (m, 1H), 1.74-1.57 (m, 1H), | |
| | | 1.20-1.08 (m, 1H), 0.94-0.79 (m, 1H). | |
| 128 | ESI-MS m/z calc. | ¹H HMR (400 MHz, CD₃OD) δ | |
| | 645.1434, found | 7.76 (s, 1H), 7.42 (d, J = 8.5 Hz, | |
| | 646.3 (M + 1)⁺; | 2H), 7.21 (d, J = 8.3 Hz, 2H), 6.29 | |
| | Retention time: | (t, J = 3.0 Hz, 1H), 5.05 (d, J = 15.1 | |
| | 1.9 minutes (LC | Hz, 1H), 4.60 (d, J = 15.2 Hz, 1H), | |
| | Method A) | 3.30-3.18 (m, 1H), 3.07 (m, 1H), | |
| | | 2.44 (m, 2H), 2.24 (m, 1H), 1.97 | |
| | | (m, 1H), 1.80 (m, 1H), 1.71-1.59 | |
| | | (m, 1H), 1.16 (m, 1H), 0.88 (dd, J = | |
| | | 9.9, 4.9 Hz, 1H). | |
| 129 | ESI-MS m/z calc. | ¹H HMR (400 MHz, methanol-d₄) δ | |
| | 571.2018, found | 7.76 (d, J = 2.2 Hz, 1H), 7.39-7.24 | |
| | 572.22 (M + 1)⁺; | (d, J = 8.4 Hz, 2H), 7.19-7.11 (m, | |
| | Retention time: | 2H), 5.03 (d, J = 15.0 Hz, 1H), 4.55 | |
| | 0.8 minutes (LC | (m, 1H), 3.18 (m, 1H), 3.03 (m, | |
| | Method S) | 1H), 2.57 (m, 2H), 2.43 (m, 2H), | |
| | | 2.23 (m, 1H), 1.94 (m, 1H), 1.84- | |
| | | 1.74 (m, 1H), 1.63 (m, 3H), 1.13 | |
| | | (m, 1H), 0.92 (m, 3H), 0.87-0.75 | |
| | | (m, 1H). | |
| 130 | ESI-MS m/z calc. | ¹H HMR (400 MHz, DMSO-d₆) δ | |
| | 577.156, found | 7.82 (s, 1H), 7.74 (s, 1H), 7.29 (s, | |
| | 578.16 (M + 1)⁺; | 2H), 7.17 (dd, J = 11.5, 8.2 Hz, | |
| | Retention time: | 1H), 7.10 (dd, J = 8.5, 2.0 Hz, 1H), | |
| | 1.75 minutes (LC | 6.93-6.83 (m, 1H), 4.88 (d, J = | |
| | Method A) | 14.9 Hz, 1H), 4.48 (d, J = 15.1 Hz, | |
| | | 1H), 3.80 (s, 3H), 3.19-3.05 (m, | |
| | | 1H), 2.98-2.89 (m, 1H), 2.35- | |
| | | 2.23 (m, 2H), 2.23-2.13 (m, 1H), | |
| | | 1.99-1.89 (m, 1H), 1.72-1.65 (m, | |
| | | 1H), 1.60-1.48 (m, 1H), 1.11- | |
| | | 1.00 (m, 1H), 0.90-0.77 (m, 1H). | |
| 131 | ESI-MS m/z calc. | ¹H HMR (400 MHz, DMSO) δ 7.82 | |
| | 577.156, found | (s, 1H), 7.71 (s, 1H), 7.27 (s, 2H), | |
| | 578.2 (M + 1)⁺; | 7.09 (dd, J = 8.5, 6.8 Hz, 1H), 6.94 | |
| | Retention time: | (dd, J = 11.3, 2.5 Hz, 1H), 6.74 (m, | |
| | 1.81 minutes (LC | 1H), 4.74 (d, J = 15.6 Hz, 1H), 4.48 | |
| | Method A) | (d, J = 15.6 Hz, 1H), 3.84 (s, 3H), | |
| | | 3.24-3.10 (m, 1H), 3.06-2.90 (m, | |
| | | 1H), 2.34-2.23 (m, 2H), 2.22- | |
| | | 2.12 (m, 1H), 1.94-1.81 (m, 1H), | |
| | | 1.74-1.61 (m, 1H), 1.60-1.47 (m, | |
| | | 1H), 1.14-0.99 (m, 1H), 0.88- | |
| | | 0.83 (m, 1H). | |
| 132 | ESI-MS m/z calc. | ¹H HMR (400 MHz, CD₃OD) δ | |
| | 627.15283, found | 7.76 (s, 1H), 7.31 (d, J = 2.1 Hz, | |
| | 628.3 (M + 1)⁺; | 1H), 7.28-7.16 (m, 2H), 5.02 (d, J = | |
| | Retention time: | 15.2 Hz, 1H), 4.55 (d, J = 15.2 | |
| | 2.07 minutes (LC | Hz, 1H), 3.22 (m, 1H), 3.05 (m, | |
| | Method A) | 1H), 2.45 (m, 1H), 2.50-2.39 (m, | |
| | | 1H), 2.29 (s, 3H), 2.24 (m, 1H), | |
| | | 1.96 (m, 1H), 1.79 (m, 1H), 1.66 | |
| | | (m, 1H), 1.16 (m, 1H), 0.88 (m, 1H). | |
| 133 | ESI-MS m/z calc. | ¹H HMR (400 MHz, DMSO-d₆) δ | |
| | 605.18616, found | 7.83 (s, 1H), 7.74 (s, 1H), 7.69- | |
| | 606.3 (M + 1)⁺; | 7.58 (m, 3H), 7.58-7.52 (m, 1H), | |
| | Retention time: | 7.51-7.40 (m, 3H), 7.40-7.34 (m, | |
| | 2.05 minutes (LC | 1H), 7.34-7.21 (m, 2H), 4.98 (d, J = | |
| | Method A) | 15.1 Hz, 1H), 4.61 (d, J = 15.2 | |
| | | Hz, 1H), 3.24-3.13 (m, 1H), 3.08- | |
| | | 3.01 (m, 1H), 2.36-2.24 (m, | |
| | | 2H), 2.24-2.11 (m, 1H), 2.02- | |
| | | 1.90 (m, 1H), 1.75-1.63 (m, 1H), | |
| | | 1.60-1.50 (m, 1H), 1.11-0.99 (m, | |
| | | 1H), 0.87-0.76 (m, 1H). | |
| 134 | ESI-MS m/z calc. | ¹H HMR (400 MHz, DMSO-d₆) δ | |
| | 545.16, found | 8.48 (s, 1H), 8.42 (s, 1H), 7.82 (s, | |
| | 546.2.3 (M + 1)⁺; | 1H), 7.29 (s, 2H), 4.87 (d, J = 15.6 | |
| | Retention time: | Hz, 1H), 4.62 (d, J = 15.6 Hz, 1H), | |
| | 1.41 minutes (LC | 3.41-3.26 (m, 1H), 3.25-3.13 (m, | |
| | Method A) | 1H), 2.48 (s, 3H), 2.36-2.23 (m, | |
| | | 2H), 2.23-2.13 (m, 1H), 2.03- | |
| | | 1.87 (m, 1H), 1.72-1.61 (m, 1H), | |
| | | 1.61-1.45 (m, 1H), 1.19-1.02 (m, | |
| | | 1H), 0.98-0.80 (m, 1H). | |
| 135 | ESI-MS m/z calc. | ¹H HMR (400 MHz, methanol-d₄) δ | |
| | 571.2018, found | 7.76 (s, 1H), 7.25-7.19 (m, 3H), | |
| | 572.3 (M + 1)⁺; | 7.17-7.12 (m, 1H), 5.07 (d, J = | |
| | Retention time: | 15.4 Hz, 1H), 4.68 (d, J = 15.4 Hz, | |
| | 2.07 minutes (LC | 1H), 3.24-3.15 (m, 1H), 3.06- | |
| | Method A) | 2.91 (m, 1H), 2.72-2.61 (m, 2H), | |
| | | 2.50-2.39 (m, 2H), 2.26-2.17 (m, | |
| | | 1H), 1.99-1.83 (m, 1H), 1.83- | |
| | | 1.72 (m, 1H), 1.72-1.56 (m, 3H), | |
| | | 1.13-1.05 (m, 1H), 1.01 (t, J = 7.3 | |
| | | Hz, 3H), 0.92-0.74 (m, 1H). | |
| 136 | ESI-MS m/z calc. | ¹H HMR (400 MHz, DMSO-d₆) δ | |
| | 545.16, found | 8.59 (s, 2H), 7.81 (s, 1H), 7.77- | |
| | 546.2.3 (M + 1)⁺; | 7.67 (m, 1H), 7.35-7.22 (m, 2H), | |
| | Retention time: | 4.87 (d, J = 16.4 Hz, 1H), 4.69 (d, J = | |
| | 1.38 minutes (LC | 16.4 Hz, 1H), 3.32-3.21 (m, | |
| | Method A) | 2H), 2.34-2.23 (m, 5H), 2.23- | |
| | | 2.17 (m, 1H), 1.96-1.87 (m, 1H), | |
| | | 1.67-1.60 (m, 1H), 1.57-1.52 (m, | |
| | | 1H), 1.14-1.06 (m, 1H), 0.94- | |
| | | 0.86 (m, 1H). | |
| 137 | ESI-MS m/z calc. | ¹H HMR (400 MHz, DMSO-d₆) δ | |
| | 599.2331, found | 7.82 (s, 1H), 7.73 (s, 1H), 7.28 (s, | |
| | 600.4 (M + 1)⁺; | 2H), 7.20 (d, J = 7.8 Hz, 2H), 7.09 | |
| | Retention time: | (d, J = 7.9 Hz, 2H), 4.83 (d, J = | |
| | 2.27 minutes (LC | 14.9 Hz, 1H), 4.55 (d, J = 15.0 Hz, | |
| | Method A) | 1H), 3.17-3.05 (m, 1H), 2.99- | |
| | | 2.91 (m, 1H), 2.45 (s, 2H), 2.31- | |
| | | 2.09 (m, 3H), 1.89-1.79 (m, 1H), | |
| | | 1.71-1.60 (m, 1H), 1.56-1.45 (m, | |
| | | 1H), 1.06-0.96 (m, 1H), 0.85 (s, | |
| | | 9H), 0.77-0.69 (m, 1H). | |
| 138 | ESI-MS m/z calc. | ¹H HMR (400 MHz, DMSO-d₆) δ | |
| | 587.21, found | 8.68 (s, 2H), 7.81 (s, 1H), 7.72 (s, | |
| | 588.4 (M + 1)⁺; | 1H), 7.29 (s, 2H), 4.80 (d, J = 15.2 | |
| | Retention time: | Hz, 1H), 4.54 (d, J = 15.3 Hz, 1H), | |
| | 1.78 minutes (LC | 3.25-3.18 (m, 1H), 3.14-3.06 (m, | |
| | Method A) | 1H), 2.31-2.24 (m, 2H), 2.22- | |
| | | 2.15 (m, 1H), 1.97-1.93 (m, 1H), | |
| | | 1.72-1.63 (m, 1H), 1.60-1.51 (m, | |
| | | 1H), 1.34 (s, 9H), 1.13-1.04 (m, | |
| | | 1H), 0.91-0.84 (m, 1H). | |
| 139 | ESI-MS m/z calc. | ¹H HMR (400 MHz, DMSO-d₆) δ | |
| | 587.1967, found | 7.83 (s, 1H), 7.74 (s, 1H), 7.29 (s, | |
| | 588.3 (M + 1)⁺; | 2H), 7.22 (t, J = 8.0 Hz, 1H), 6.90- | |
| | Retention time: | 6.74 (m, 3H), 4.85 (d, J = 15.1 Hz, | |
| | 1.94 minutes (LC | 1H), 4.59-4.45 (m, 2H), 3.20- | |
| | Method A) | 3.08 (m, 1H), 2.99-2.90 (m, 1H), | |
| | | 2.34-2.22 (m, 2H), 2.22-2.11 (m, | |
| | | 1H), 1.97-1.82 (m, 1H), 1.74- | |
| | | 1.64 (m, 1H), 1.59-1.48 (m, 1H), | |
| | | 1.24 (dd, J = 6.1, 2.6 Hz, 6H), 1.09- | |
| | | 0.99 (m, 1H), 0.83-0.73 (m, 1H). | |
| 140 | ESI-MS m/z calc. | | |
| | 605.18616, found | | |
| | 606.3 (M + 1)⁺; | | |
| | Retention time: | | |
| | 2.08 minutes (LC | | |
| | Method A) | | |
| 141 | ESI-MS m/z calc. | | |
| | 467.13922, found | | |
| | 468.2 (M + 1)⁺; | | |
| | Retention time: | | |
| | 1.27 minutes (LC | | |
| | Method A) | | |
| 142 | ESI-MS m/z calc. | | |
| | 595.1466, found | | |
| | 596.17 (M + 1)⁺; | | |
| | Retention time: | | |
| | 1.8 minutes (LC | | |
| | Method J) | | |
| 143 | ESI-MS m/z calc. | | |
| | 577.156, found | | |
| | 578.16 (M + 1)⁺; | | |
| | Retention time: | | |
| | 1.14 minutes (LC | | |
| | Method J) | | |
| 144 | ESI-MS m/z calc. | | |
| | 577.156, found | | |
| | 578.2 (M + 1)⁺; | | |
| | Retention time: | | |
| | 1.79 minutes (LC | | |
| | Method J) | | |
| 145 | ESI-MS m/z calc. | ¹H HMR (400 MHz, methanol-d₄) δ | |
| | 647.0982, found | 7.77 (s, 1H), 7.41 (s, 1H), 7.25 (d, J = | |
| | 648.4 (M + 1)⁺; | 7.1 Hz, 2H), 4.99 (d, J = 15.7 Hz, | |
| | Retention time: | 1H), 4.66 (d, J = 15.6 Hz, 1H), 3.09 | |
| | 2.1 minutes (LC | (m, 1H), 2.45 (m, 2H), 2.24 (m, | |
| | Method A) | 1H), 1.95 (m, 1H), 1.81 (m, 1H), | |
| | | 1.69-1.62 (m, 1H), 1.19 (m, 1H), | |
| | | 0.95-0.84 (m, 1H), 0.88 (m, 1H). | |
| 146 | ESI-MS m/z calc. | | |
| | 627.15283, found | | |
| | 628.2 (M + 1)⁺; | | |
| | Retention time: | | |
| | 1.89 minutes (LC | | |
| | Method A) | | |
| 147 | ESI-MS m/z calc. | ¹H HMR (400 MHz, methanol-d₄) δ | |
| | 627.15283, found | 7.77 (s, 1H), 7.30-7.13 (m, 3H), | |
| | 628.2 (M + 1)⁺; | 5.01 (d, J = 15.8 Hz, 1H), 4.71 (d, J = | |
| | Retention time: | 15.8 Hz, 1H), 3.26 (d, J = 3.6 Hz, | |
| | 2.06 minutes (LC | 1H), 3.06 (m, 1H), 2.46 (m, 2H), | |
| | Method A) | 2.31 (s, 3H), 2.25 (m, 1H), 1.97- | |
| | | 1.86 (m, 1H), 1.79 (dd, J = 9.9, 5.0 | |
| | | Hz, 1H), 1.72-1.54 (m, 1H), 1.12 | |
| | | (m, 1H), 0.93-0.77 (m, 1H). | |
| 148 | ESI-MS m/z calc. | ¹H HMR (400 MHz, DMSO) δ 7.83 | |
| | 627.15283, found | (s, 1H), 7.72 (s, 1H), 7.29 (s, 2H), | |
| | 628.3 (M + 1)⁺; | 7.17 (s, 1H), 7.07 (d, J = 10.7 Hz, | |
| | Retention time: | 2H), 4.85 (d, J = 15.4 Hz, 1H), 4.57 | |
| | 2.05 minutes (LC | (d, J = 15.4 Hz, 1H), 3.24-3.11 | |
| | Method A) | (m, 1H), 3.04-2.91 (m, 1H), 2.33 | |
| | | (s, 2H), 2.32-2.24 (m, 2H), 2.21- | |
| | | 2.11 (m, 1H), 1.95-1.84 (m, 1H), | |
| | | 1.73-1.63 (m, 1H), 1.58-1.48 (m, | |
| | | 1H), 1.13-1.01 (m, 1H), 0.87- | |
| | | 0.73 (m, 1H). | |
| 149 | ESI-MS m/z calc. | | |
| | 597.1423, found | | |
| | 598.11 (M + 1)⁺; | | |
| | Retention time: | | |
| | 0.59 minutes (LC | | |
| | Method M) | | |
| 150 | ESI-MS m/z calc. | ¹H HMR (400 MHz, methanol-d₄) δ | |
| | 597.21747, found | 7.75 (s, 1H), 7.33-7.10 (m, 4H), | |
| | 598.19 (M + 1)⁺; | 5.05 (d, J = 14.9 Hz, 1H), 4.51 (d, J = | |
| | Retention time: | 14.9 Hz, 1H), 3.61 (p, J = 8.2 Hz, | |
| | 2.21 minutes (LC | 1H), 3.23-3.10 (m, 1H), 3.02 (m, | |
| | Method A) | 1H), 2.52-2.34 (m, 3H), 2.26 (m, | |
| | | 3H), 2.08-1.90 (m, 3H), 1.78 (m, | |
| | | 1H), 1.69-1.54 (m, 1H), 1.23 (d, J = | |
| | | 6.9 Hz, 3H), 1.12 (m, 1H), 0.95- | |
| | | 0.73 (m, 1H). | |
| 151 | ESI-MS m/z calc. | | |
| | 586.2127, found | | |
| | 587.2 (M + 1)⁺; | | |
| | Retention time: | | |
| | 1.47 minutes (LC | | |
| | Method A) | | |
| 152 | ESI-MS m/z calc. | ¹H HMR (400 MHz, methanol-d₄) δ | |
| | 613.22363, found | 7.95 (dd, J = 9.4, 7.1 Hz, 1H), 7.78 | |
| | 614.3 (M + 1)⁺; | (d, J = 3.6 Hz, 1H), 7.34 (d, J = 9.4 | |
| | Retention time: | Hz, 1H), 6.94 (d, J = 7.3 Hz, 1H), | |
| | 1.32 minutes (LC | 4.98 (d, J = 15.4 Hz, 1H), 4.70 (d, J = | |
| | Method A) | 15.5 Hz, 1H), 3.75 (s, 4H), 3.45 | |
| | | (s, 1H), 3.35 (s, 1H), 3.29-3.20 | |
| | | (m, 1H), 2.48 (m, 2H), 2.27 (m, | |
| | | 1H), 2.13-1.98 (m, 1H), 1.81 (s, | |
| | | 6H), 1.72 (s, 1H), 1.36-1.20 (m, | |
| | | 1H), 1.00 (m, 1H). | |
| 153 | ESI-MS m/z calc. | ¹H HMR (400 MHz, DMSO) δ 7.83 | |
| | 627.15283, found | (s, 1H), 7.71 (s, 1H), 7.36-7.20 | |
| | 628.3 (M + 1)⁺; | (m, 3H), 7.01-6.91 (m, 3H), 4.87 | |
| | Retention time: | (d, J = 15.2 Hz, 1H), 4.71 (q, J = | |
| | 1.88 minutes (LC | 8.9 Hz, 2H), 4.51 (d, J = 15.3 Hz, | |
| | Method A) | 1H), 3.18-3.09 (m, 1H), 3.03- | |
| | | 2.90 (m, 1H), 2.34-2.23 (m, 2H), | |
| | | 2.23-2.13 (m, 1H), 1.97-1.85 (m, | |
| | | 1H), 1.74-1.63 (m, 1H), 1.60- | |
| | | 1.47 (m, 1H), 1.10-0.97 (m, 1H), | |
| | | 0.89-0.76 (m, 1H). | |
| 154 | ESI-MS m/z calc. | ¹H HMR (400 MHz, methanol-d₄) δ | |
| | 613.22363, found | 8.05 (m, 1H), 7.90 (dd, J = 10.0, 2.1 | |
| | 614.3 (M + 1)⁺; | Hz, 1H), 7.76 (d, J = 3.5 Hz, 1H), | |
| | Retention time: | 7.41 (dd, J = 9.6, 4.6 Hz, 1H), 4.54 | |
| | 1.2 minutes (LC | (d, J = 15.2 Hz, 1H), 3.70 (t, J = 4.9 | |
| | Method A) | Hz, 4H), 3.47-3.32 (m, 1H), 3.28- | |
| | | 3.10 (m, 1H), 2.45 (m, 2H), 2.25 | |
| | | (m, 1H), 1.77 (d, 6H), 1.31-1.16 | |
| | | (m, 1H), 0.95 (m, 1H). | |

### Example 155: Preparation of (6R)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 188)

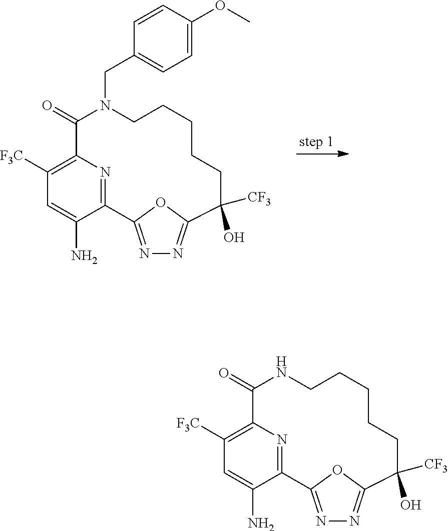 [SMILES: COc1ccc(CN2CCCCC[C@](O)(C(F)(F)F)C3=NN=C(O3)c3nc(c(C(F)(F)F)cc3N)C2=O)cc1 ; Nc1cc(C(F)(F)F)c2nc1C1=NN=C(O1)[C@@](O)(C(F)(F)F)CCCCCNC2=O]

Step 1: (6R)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 188) (6R)-17-Amino-6-hydroxy-12-[(4-methoxyphenyl)methyl]-6,15-bis(trifluoromethyl)-19-oxa-3,4,12,18-tetrazatricyclo[12.3.1.12,5]nonadeca-1(18),2,4,14,16-pentaen-13-one (42.6 mg, 0.07614 mmol), TFA (1 mL), water (50 μL) and triisopropylsilane (50 μL, 0.2441 mmol) was stirred at room temperature for 5 h, then the solvent was evaporated. The residue was dissolved in ethyl acetate and washed with 1 M NaHCO₃, dried (MgSO₄), filtered and evaporated. The residue was purified by silica gel chromatography using a gradient from 10% to 50% ethyl acetate in hexanes over 15 min which gave as a white solid, (6R)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (30.8 mg, 92%). ¹H NMR (400 MHz, CDCl₃) δ 8.24 (s, 1H), 7.54 (d, J=6.7 Hz, 1H), 6.10-5.72 (m, 2H), 3.70 (d, J=2.5 Hz, 1H), 3.56-3.03 (m, 2H), 2.53-2.20 (m, 2H), 2.05 (s, 1H), 1.79 (m, 3H), 1.42 (dd, J=13.5, 7.7 Hz, 1H), 1.18 (m, 1H) ppm. ¹⁹F NMR (376 MHz, CDCl₃) δ−60.28, −60.50, −78.44, −80.03 ppm. ESI-MS m/z calc. 439.1079, found 440.0 (M+1)⁺; Retention time: 0.97 minutes (LC Method Q).

### Example 156: Preparation of (6S)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 189)

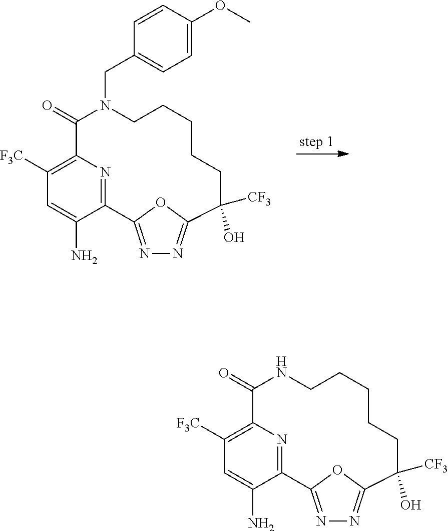 [SMILES: COc1ccc(CN2CCCCC[C@@](O)(C(F)(F)F)C3=NN=C(O3)c3nc(c(C(F)(F)F)cc3N)C2=O)cc1 ; Nc1cc(C(F)(F)F)c2nc1C1=NN=C(O1)[C@](O)(C(F)(F)F)CCCCCNC2=O]

Step 1: (6S)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 189) (6S)-17-Amino-6-hydroxy-12-[(4-methoxyphenyl)methyl]-6,15-bis(trifluoromethyl)-19-oxa-3,4,12,18-tetrazatricyclo[12.3.1.12,5]nonadeca-1(18),2,4,14,16-pentaen-13-one (41 mg, 0.07328 mmol) was subjected to the similar procedure described for Example 155 to provide (6S)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (30.5 mg, 95%). ¹H NMR (400 MHz, CDCl₃) δ 8.24 (s, 1H), 7.58-7.52 (m, 1H), 6.12-5.66 (m, 2H), 3.78-3.71 (m, 1H), 3.56-3.04 (m, 2H), 2.52-2.20 (m, 2H), 2.12-1.99 (m, 1H), 1.96-1.64 (m, 3H), 1.42 (dd, J=13.8, 7.9 Hz, 1H), 1.22-1.13 (m, 1H) ppm. ¹⁹F NMR (376 MHz, Chloroform-d) δ−60.28, −60.50, −78.44, −80.03 ppm. ESI-MS m/z calc. 439.1079, found 440.1 (M+1)⁺; Retention time: 0.97 minutes (LC Method Q).

### Example 157: Preparation of (6R)-12-(2-acetyl-2-azaspiro[3.3]heptan-6-yl)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 190)

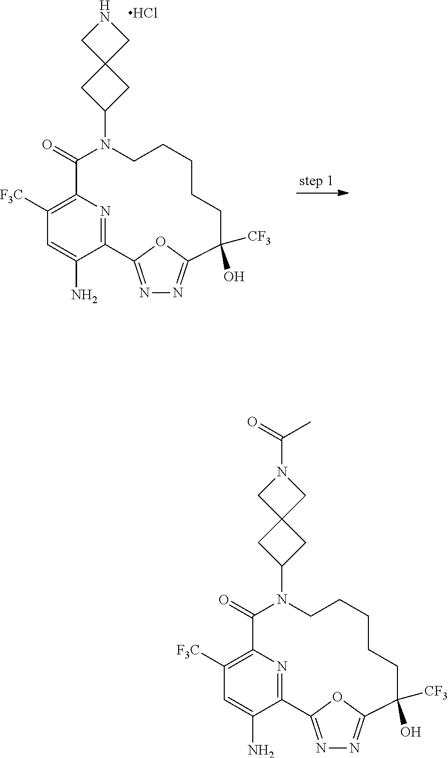 [SMILES: Cl ; Nc1cc(C(F)(F)F)c2nc1C1=NN=C(O1)[C@@](O)(C(F)(F)F)CCCCCN(C1CC3(CNC3)C1)C2=O ; CC(=O)N1CC2(CC(N3CCCCC[C@](O)(C(F)(F)F)C4=NN=C(O4)c4nc(c(C(F)(F)F)cc4N)C3=O)C2)C1]

### Step 1: (6R)-12-(2-acetyl-2-azaspiro[3.3]heptan-6-yl)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 190)

HATU (19 mg, 0.04997 mmol), DIEA (17 μL, 0.0976 mmol) and acetic acid (25 μL of a 2 M solution in THF, 0.05 mmol) were dissolved in DMF (0.166 mL). Part of this solution (51 μL) was added to a mixture of (6R)-17-amino-12-(2-azaspiro[3.3]heptan-6-yl)-6-hydroxy-6,15-bis(trifluoromethyl)-19-oxa-3,4,12,18-tetrazatricyclo[12.3.1.12,5]nonadeca-1(18),2,4,14,16-pentaen-13-one (hydrochloride salt) (9 mg, 0.01684 mmol) in DMF (90 μL). The mixture was stirred at room temperature for 30 min, then diluted with ether, washed with water (2×), dried (MgSO₄), filtered and evaporated. The residue was purified by silica gel chromatography using a gradient from 5% to 100% ethyl acetate in hexanes over 20 min to provide as a white solid, (6R)-12-(2-acetyl-2-azaspiro[3.3]heptan-6-yl)-17-amino-6-hydroxy-6,15-bis(trifluoromethyl)-19-oxa-3,4,12,18-tetrazatricyclo[12.3.1.12,5]nonadeca-1(18),2,4,14,16-pentaen-13-one (4.2 mg, 41%). ¹H NMR (400 MHz, methanol-d4) δ 7.62 (s, 1H), 4.40-4.24 (m, 1H), 4.22 (s, 1H), 4.10 (s, 1H), 3.98 (s, 1H), 3.85 (s, 1H), 3.08 (m, 2H), 2.63-2.51 (m, 2H), 2.49-2.38 (m, 1H), 2.27 (d, J=14.5 Hz, 2H), 2.16 (s, 1H), 1.78-1.74 (m, 3H), 1.69 (s, 2H), 1.58 (s, 2H), 1.09 (m, 1H), 0.87-0.73 (m, 1H) ppm; ¹⁹F NMR (376 MHz, methanol-d4) δ−61.78 (d, J=5.6 Hz), −81.13 ppm. ESI-MS m/z calc. 576.19196, found 577.3 (M+1)⁺; Retention time: 1.01 minutes (LC Method Q).

### Example 158: Preparation of (6R)-17-amino-12-[2-(cyclopropanecarbonyl)-2-azaspiro[3.3]heptan-6-yl]-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 191)

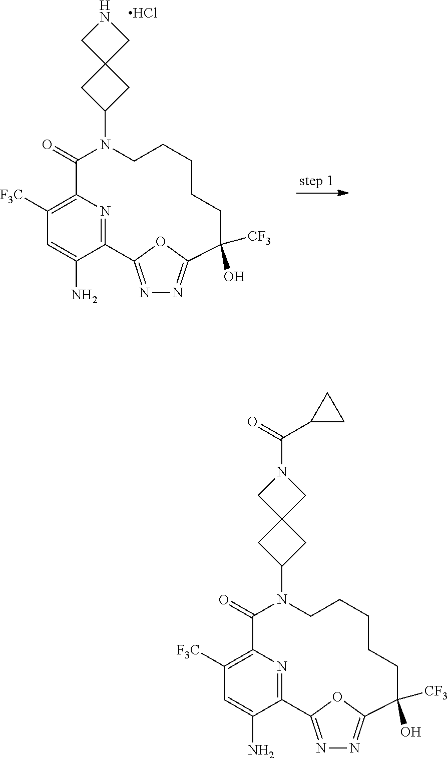 [SMILES: Cl ; Nc1cc(C(F)(F)F)c2nc1C1=NN=C(O1)[C@@](O)(C(F)(F)F)CCCCCN(C1CC3(CNC3)C1)C2=O ; Nc1cc(C(F)(F)F)c2nc1C1=NN=C(O1)[C@@](O)(C(F)(F)F)CCCCCN(C1CC3(C1)CN(C(=O)C1CC1)C3)C2=O]

Step 1: (6R)-17-Amino-12-[2-(cyclopropanecarbonyl)-2-azaspiro[3.3]heptan-6-yl]-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 191) HATU (19 mg, 0.04997 mmol), DIEA (17 μL, 0.0976 mmol) and cyclopropanecarboxylic acid (4 μL, 0.05018 mmol) were dissolved in DMF (0.127 mL). Part of this solution (51 μL) was added to a mixture of (6R)-17-amino-12-(2-azaspiro[3.3]heptan-6-yl)-6-hydroxy-6,15-bis(trifluoromethyl)-19-oxa-3,4,12,18-tetrazatricyclo[12.3.1.12,5]nonadeca-1(18),2,4,14,16-pentaen-13-one (hydrochloride salt) (9 mg, 0.01684 mmol) in DMF (90 μL). The mixture was stirred at room temperature for 30 min then diluted with ether, washed with water (2×), dried (MgSO₄), filtered and evaporated. The residue was purified by silica gel chromatography using a gradient from 5% to 100% ethyl acetate in hexanes over 20 min to provide as a white solid, (6R)-17-amino-12-[2-(cyclopropanecarbonyl)-2-azaspiro[3.3]heptan-6-yl]-6-hydroxy-6,15-bis(trifluoromethyl)-19-oxa-3,4,12,18-tetrazatricyclo[12.3.1.12,5]nonadeca-1(18),2,4,14,16-pentaen-13-one (5.8 mg, 57%). ¹H NMR (400 MHz, methanol-d4) δ 7.74 (s, 1H), 4.46 (s, 1H), 4.44-4.35 (m, 1H), 4.33 (s, 1H), 4.11 (s, 1H), 3.98 (s, 1H), 3.25-3.11 (m, 2H), 2.67 (m, 2H), 2.60-2.48 (m, 1H), 2.41 (s, 2H), 2.27 (s, 1H), 1.82 (s, 2H), 1.75-1.67 (m, 1H), 1.58 (m, 1H), 1.26-1.16 (m, 1H), 1.00-0.76 (m, 6H) ppm; ¹⁹F NMR (376 MHz, methanol-d4) δ−61.77 (d, J=6.2 Hz), −81.15 ppm; ESI-MS m/z calc. 602.20764, found 603.3 (M+1)⁺; Retention time: 1.18 minutes (LC Method Q).

### Example 159: Preparation of (6R)-17-amino-6-hydroxy-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-13-one (hydrochloride salt) (Compound 192) and trans-4-[(6R)-17-amino-6-hydroxy-13-oxo-6,15-bis(trifluoromethyl)-19-oxa-3,4,12,18-tetraazatricyclo[12.3.1.12,5]nonadeca-1(18),2,4,14,16-pentaen-12-yl]cyclohexane-1-carbonitrile (Compound 193)

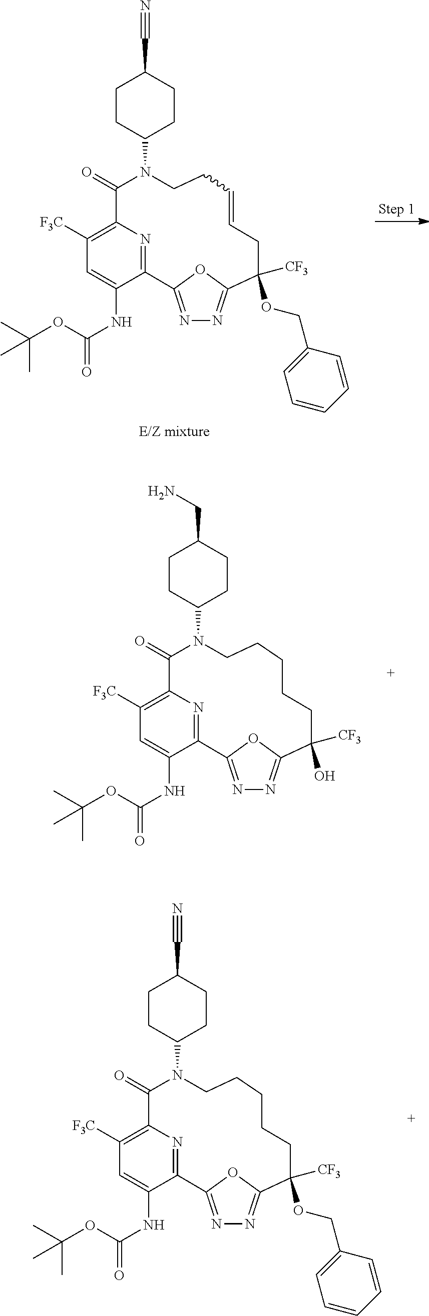

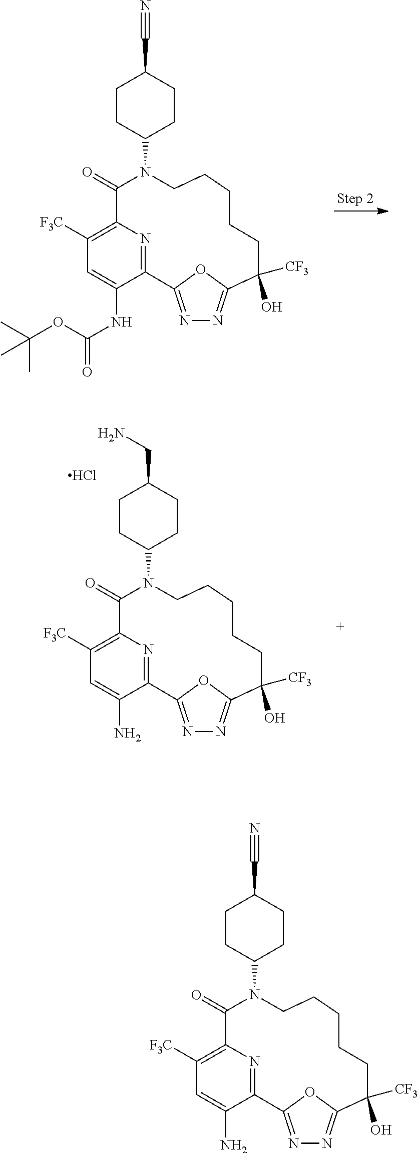

### Step 1: tert-Butyl N-[(6R)-6-hydroxy-13-oxo-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate, tert-butyl N-[(6R)-6-(benzyloxy)-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate and tert-butyl N-[(6R)-6-hydroxy-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate

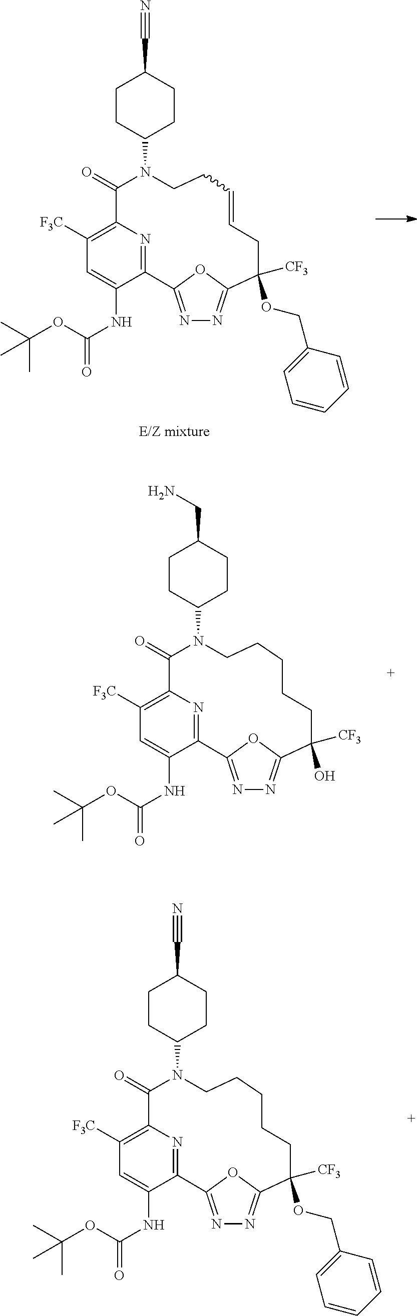

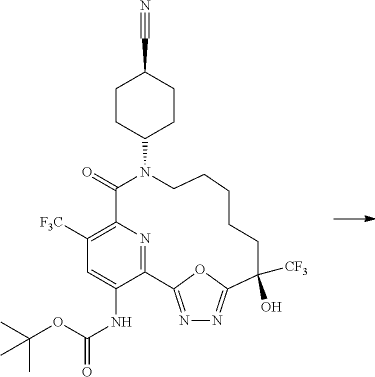

A mixture of tert-butyl N-[(6R)-6-(benzyloxy)-13-oxo-12-[(1r,4r)-4-cyanocyclohexyl]-6,15-bis(trifluoromethyl)-19-oxa-3,4,12,18-tetraazatricyclo[12.3.1.12,5]nonadeca-1(18),2,4,8,14,16-hexaen-17-yl]carbamate (E/Z mixture) (20.6 mg, 0.02804 mmol) (prepared from 6-[5-[(1R)-1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid and trans-4-[(but-3-en-1-yl)amino]cyclohexane-1-carbonitrile (hydrochloride salt) (prepared by method 8) using steps 1 and 2 of Method 13) and Pd/C (9 mg of 10% w/w, 0.008457 mmol) in acetic acid (450 μL) was stirred at room temperature under 200 psi H₂ in a stainless steel pressure vessel for 14 h. The mixture was then filtered and the filtrate was evaporated to provide a mixture of tert-butyl N-[(6R)-6-hydroxy-13-oxo-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate (ESI-MS m/z calc. 650.26514, found 651.0 (M+1)⁺; Retention time: 0.56 minutes (LC Method T), tert-butyl N-[(6R)-6-(benzyloxy)-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate (Retention time: 0.74 minutes (LC Method T)) and tert-butyl N-[(6R)-6-hydroxy-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate (ESI-MS m/z calc. 646.2338, found 647.0 (M+1)⁺; Retention time: 0.72 minutes (LC Method T). This mixture was taken directly to the next step.

### Step 2: (6R)-17-Amino-6-hydroxy-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-13-one (hydrochloride salt) (Compound 192) and trans-4-[(6R)-17-amino-6-hydroxy-13-oxo-6,15-bis(trifluoromethyl)-19-oxa-3,4,12,18-tetraazatricyclo[12.3.1.12,5]nonadeca-1(18),2,4,14,16-pentaen-12-yl]cyclohexane-1-carbonitrile (Compound 193)

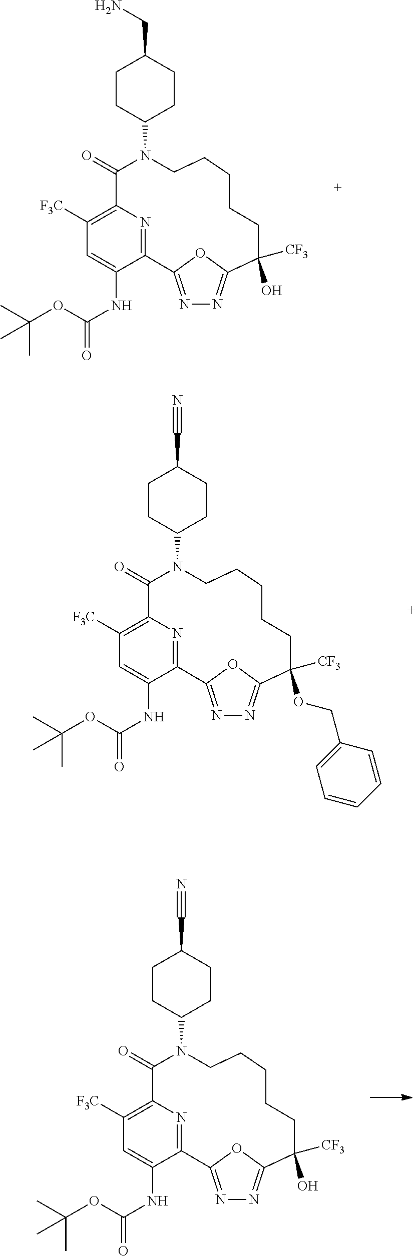

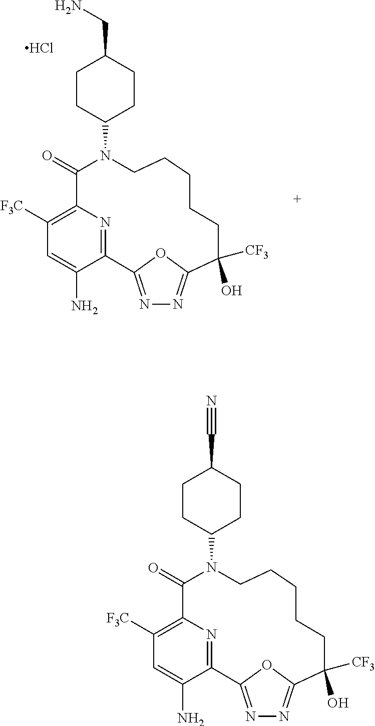

The crude mixture of tert-butyl N-[(6R)-6-hydroxy-13-oxo-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate, tert-butyl N-[(6R)-6-(benzyloxy)-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate and tert-butyl N-[(6R)-6-hydroxy-13-oxo-12-[trans-4-cyanocyclohexyl]-6,15-bis(trifluoromethyl)-19-oxa-3,4,12,18-tetraazatricyclo[12.3.1.12,5]nonadeca-1(18),2,4,14,16-pentaen-17-yl]carbamate was stirred in a mixture of TFA (300 μL), water (15 μL) and triisopropylsilane (15 μL, 0.07322 mmol) at room temperature for 30 min and then the solvent was evaporated. The residue was dissolved into ethyl acetate and washed with 1 M NaHCO₃, dried (MgSO₄), filtered and evaporated. The residue was subjected to preparative HPLC eluting with a gradient from 1% to 80% acetonitrile in 5 mM aqueous HCl at 50 mL/min over 14 min through a Luna 5 μM C₁₈ 100 Å, 75×30 mm column to provide as the first diastereomer to elute and a white solid, (6R)-17-amino-6-hydroxy-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-13-one (hydrochloride salt) (4.7 mg, 29%). ¹H NMR (400 MHz, methanol-d4) δ 7.75 (s, 1H), 4.08-3.93 (m, 1H), 3.26-2.99 (m, 2H), 2.86 (d, J=6.9 Hz, 2H), 2.47 (s, 2H), 2.31-2.19 (m, 1H), 2.10-1.81 (m, 8H), 1.77-1.57 (m, 2H), 1.34-1.23 (m, 2H), 1.15-1.05 (m, 1H), 0.88-0.73 (m, 1H) ppm; ¹⁹F NMR (376 MHz, methanol-d4) δ−61.68, −81.20 ppm; ESI-MS m/z calc. 550.2127, found 551.2 (M+1)⁺; Retention time: 0.82 minutes (LC Method Q).

The second diastereomer to elute was concentrated to give as a white solid, trans-4-[(6R)-17-amino-6-hydroxy-13-oxo-6,15-bis(trifluoromethyl)-19-oxa-3,4,12,18-tetraazatricyclo[12.3.1.12,5]nonadeca-1(18),2,4,14,16-pentaen-12-yl]cyclohexane-1-carbonitrile (3.6 mg, 24%). ¹H NMR (400 MHz, methanol-d4) δ 7.75 (s, 1H), 4.05-3.93 (m, 1H), 3.22-3.02 (m, 2H), 2.66 (m, 1H), 2.52-2.39 (m, 2H), 2.30-2.22 (m, 3H), 2.09-1.96 (m, 3H), 1.90 (d, J=12.7 Hz, 2H), 1.81-1.64 (m, 4H), 1.16-1.01 (m, 1H), 0.94-0.69 (m, 1H) ppm; ¹⁹F NMR (376 MHz, methanol-d4) δ−61.67, −81.28 ppm; ESI-MS m/z calc. 546.1814, found 547.2 (M+1)⁺; Retention time: 1.21 minutes (LC Method Q).

### Example 160: Preparation of N-[[4-[(6R)-17-amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]trans-cyclohexyl]methyl]acetamide (Compound 194)

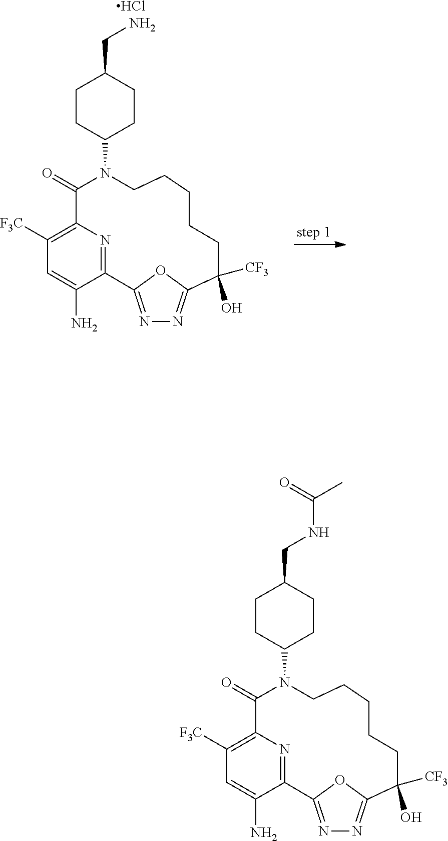

### Step 1: N-[[4-[(6R)-17-Amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]trans-cyclohexyl]methyl]acetamide (Compound 194)

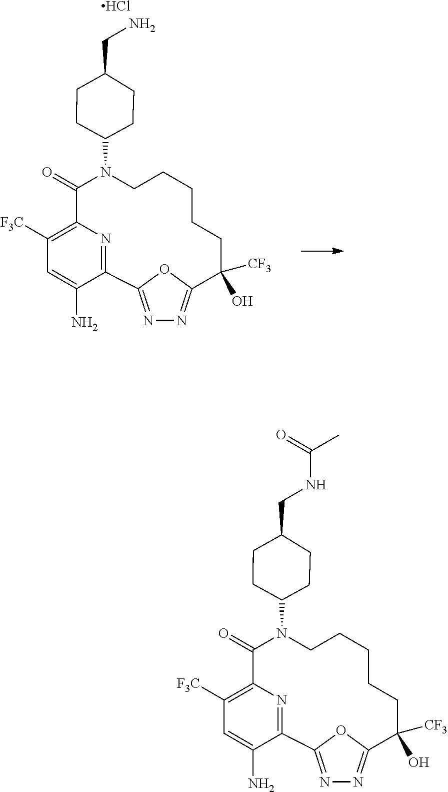

HATU (19 mg, 0.05 mmol), DIEA (24 μL, 0.14 mmol) and acetic acid (25 μL of a 2 M solution in THF, 0.05 mmol) were dissolved in 0.106 mL of DMF. Part of this solution (0.014 mL, 0.0041 mmol of acetic acid) was added to (6R)-17-amino-6-hydroxy-12-[trans-4-(aminomethyl)cyclohexyl]-6,15-bis(trifluoromethyl)-19-oxa-3,4,12,18-tetraazatricyclo[12.3.1.12,5]nonadeca-1(18),2,4,14,16-pentaen-13-one (hydrochloride salt) (2.4 mg, 0.0041 mmol) in DMF (48 μL). The mixture was stirred at room temperature for 30 min then diluted with ether, washed with water, dried (MgSO₄), filtered and evaporated. The residue was purified by silica gel chromatography using a gradient from 10% to 100% ethyl acetate in hexanes over 20 minutes which gave as a white solid, N-[[4-[(6R)-17-amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]trans-cyclohexyl]methyl]acetamide (1.2 mg, 50%). ¹H NMR (400 MHz, CD₃OD) δ 7.62 (s, 1H), 3.97 (dd, J=22.4, 7.6 Hz, 1H), 3.11-2.99 (m, 1H), 2.95 (d, J=6.8 Hz, 2H), 2.35 (s, 2H), 2.20-2.12 (m, 1H), 1.85 (s, 3H), 1.83-1.69 (m, 6H), 1.64-1.52 (m, 1H), 1.46-1.21 (m, 2H), 1.10-0.96 (m, 4H), 0.90-0.74 (m, 1H), 0.66 (s, 1H) ppm; ¹⁹F NMR (376 MHz, CD₃OD) δ−61.70, −81.25 ppm. ESI-MS m/z calc. 592.22327, found 593.2 (M+1)⁺; Retention time: 1.08 minutes (LC Method Q).

### Example 161: Preparation of (6R)-17-amino-12-[[4-(1-bicyclo[1.1.1]pentanyl)phenyl]methyl]-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 195)

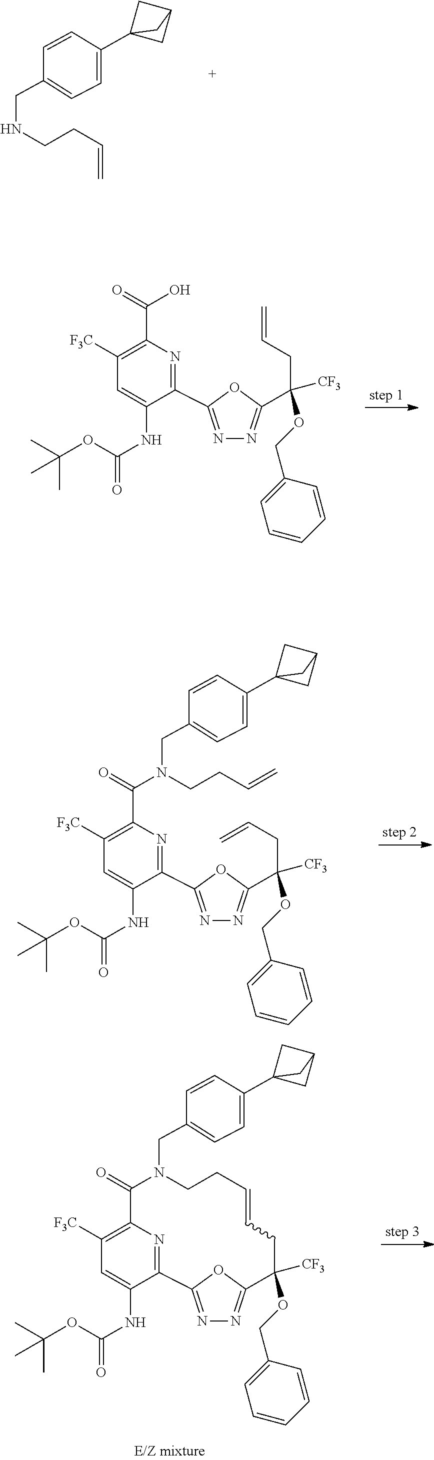

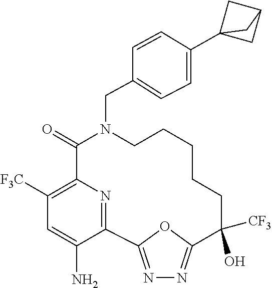 [SMILES: Nc1cc(C(F)(F)F)c2nc1-c1nnc(o1)[C@@](O)(C(F)(F)F)CCCCCN(Cc1ccc(C34CC(C3)C4)cc1)C2=O]

### Step 1: tert-Butyl N-[2-[5-[(1R)-1-benzyloxy-1-(trifluoromethyl)but-3-enyl]-1,3,4-oxadiazol-2-yl]-6-[[4-(1-bicyclo[1.1.1]pentanyl)phenyl]methyl-but-3-enyl-carbamoyl]-5-(trifluoromethyl)-3-pyridyl]carbamate

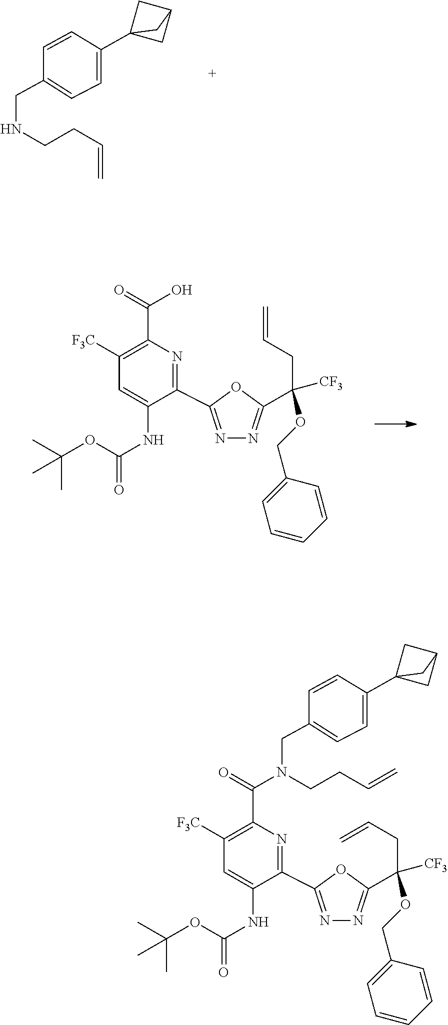 [SMILES: C=CCCNCc1ccc(C23CC(C2)C3)cc1 ; C=CC[C@@](OCc1ccccc1)(c1nnc(-c2nc(C(=O)O)c(C(F)(F)F)cc2NC(=O)OC(C)(C)C)o1)C(F)(F)F ; C=CCCN(Cc1ccc(C23CC(C2)C3)cc1)C(=O)c1nc(-c2nnc([C@@](CC=C)(OCc3ccccc3)C(F)(F)F)o2)c(NC(=O)OC(C)(C)C)cc1C(F)(F)F]

To a solution of 6-[5-[(1R)-1-benzyloxy-1-(trifluoromethyl)but-3-enyl]-1,3,4-oxadiazol-2-yl]-5-(tert-butoxycarbonylamino)-3-(trifluoromethyl)pyridine-2-carboxylic acid (53 mg, 0.08797 mmol) and N-[[4-(1-bicyclo[1.1.1]pentanyl)phenyl]methyl]but-3-en-1-amine (25 mg, 0.08797 mmol) (prepared by Method 12) in DMF (1 mL) was added DIEA (108.3 mg, 0.838 mmol) followed by HATU (159.2 mg, 0.4187 mmol). The mixture was stirred at room temperature for 15 minutes then diluted with ether, washed with water (2×), dried (MgSO₄), filtered and evaporated. The residue was purified by silica chromatography using a gradient from 0% to 25% ethyl acetate in hexanes to afford as a white solid, tert-butyl N-[2-[5-[(1R)-1-benzyloxy-1-(trifluoromethyl)but-3-enyl]-1,3,4-oxadiazol-2-yl]-6-[[4-(1-bicyclo[1.1.1]pentanyl)phenyl]methyl-but-3-enyl-carbamoyl]-5-(trifluoromethyl)-3-pyridyl]carbamate (51 mg, 71%). ESI-MS m/z calc. 811.31683, found 812.32 (M+1)⁺; Retention time: 0.87 minutes (LC Method T).

### Step 2: tert-Butyl N-[(6R)-6-benzyloxy-12-[[4-(1-bicyclo[1.1.1]pentanyl)phenyl]methyl]-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z Mixture)

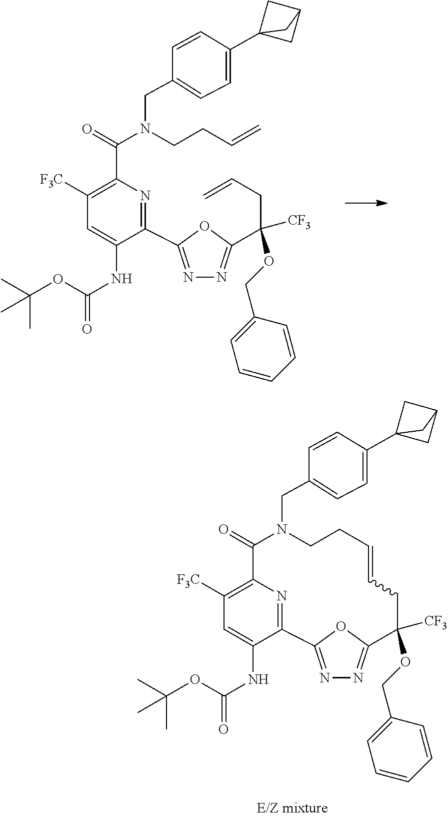 [SMILES: C=CCCN(Cc1ccc(C23CC(C2)C3)cc1)C(=O)c1nc(-c2nnc([C@@](CC=C)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1-c1nnc(o1)[C@@](OCc1ccccc1)(C(F)(F)F)CC=CCCN(Cc1ccc(C34CC(C3)C4)cc1)C2=O]

To a one neck flask with nitrogen gas bubbling in was added tert-butyl N-[2-[5-[(1R)-1-benzyloxy-1-(trifluoromethyl)but-3-enyl]-1,3,4-oxadiazol-2-yl]-6-[[4-(1-bicyclo[1.1.1]pentanyl)phenyl]methyl-but-3-enyl-carbamoyl]-5-(trifluoromethyl)-3-pyridyl]carbamate (40 mg, 0.04927 mmol) in 1,2-dichloroethane (100 mL) and the mixture was heated to 60° C. Then, dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (11.25 mg, 0.01533 mmol) in 1,2-dichloroethane (2.5 mL) was added via syringe and the resulting mixture was heated to 60° C. and stirred for 45 min. The reaction mixture was concentrated under reduced pressure and purified by silica gel chromatography using a gradient from 0% to 30% ethyl acetate in hexanes to afford as a white solid, tert-butyl N-[(6R)-6-benzyloxy-12-[[4-(1-bicyclo[1.1.1]pentanyl)phenyl]methyl]-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (34.1 mg, 88%). ESI-MS m/z calc. 783.2855, found 784.23 (M+1)⁺; Retention time: 0.87 minutes (LC Method T).

### Step 3: (6R)-17-Amino-12-[[4-(1-bicyclo[1.1.1]pentanyl)phenyl]methyl]-6-hydroxy-6,15-bis(trifluoromethyl)-19-oxa-3,4,12,18-tetrazatricyclo[12.3.1.12,5]nonadeca-1(18),2,4,14,16-pentaen-13-one (Compound 195)

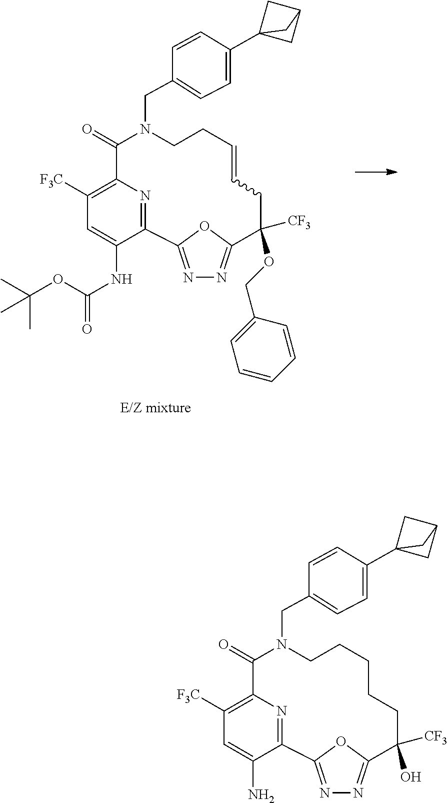 [SMILES: CC(C)(C)OC(=O)Nc1cc(C(F)(F)F)c2nc1-c1nnc(o1)[C@@](OCc1ccccc1)(C(F)(F)F)CC=CCCN(Cc1ccc(C34CC(C3)C4)cc1)C2=O ; Nc1cc(C(F)(F)F)c2nc1-c1nnc(o1)[C@@](O)(C(F)(F)F)CCCCCN(Cc1ccc(C34CC(C3)C4)cc1)C2=O]

Part 1: A mixture of tert-butyl N-[(6R)-6-benzyloxy-12-[[4-(1-bicyclo[1.1.1]pentanyl)phenyl]methyl]-13-oxo-6,15-bis(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (40 mg, 0.05104 mmol) and Pd/C (5.432 mg of 10% w/w, 0.005104 mmol) in ethyl acetate (2 mL) was stirred at room temperature under a balloon of hydrogen at atmospheric pressure. After one hour, additional Pd/C (approximately 4.052 mg of 10% w/w, 0.003808 mmol) was added and the reaction mixture was stirred at the same conditions for an additional hour. The mixture was then filtered and the filtrate was evaporated to provide crude tert-butyl N-[(6R)-6-benzyloxy-12-[[4-(3-methylcyclobutyl)phenyl]methyl]-13-oxo-6,15-bis(trifluoromethyl)-19-oxa-3,4,12,18-tetrazatricyclo[12.3.1.12,5]nonadeca-1(18),2,4,14,16-pentaen-17-yl]carbamate (20 mg, 50%) (ESI-MS m/z calc. 787.31683, found 788.41 (M+1)⁺; Retention time: 2.21 minutes (LC Method A)). This material was contaminated with tert-butyl N-[(6R)-6-benzyloxy-12-[[4-(1-bicyclo[1.1.1]pentanyl)phenyl]methyl]-13-oxo-6,15-bis(trifluoromethyl)-19-oxa-3,4,12,18-tetrazatricyclo[12.3.1.12,5]nonadeca-1(17),2,4,14(18),15-pentaen-17-yl]carbamate (10 mg, 25%) (ESI-MS m/z calc. 785.3012, found 788.41 (M+1)⁺; Retention time: 2.21 minutes (LC Method A)). This crude material was used directly in the next part without further purification.

Part 2: The mixture of tert-butyl N-[(6R)-6-benzyloxy-12-[[4-(3-methylcyclobutyl)phenyl]methyl]-13-oxo-6,15-bis(trifluoromethyl)-19-oxa-3,4,12,18-tetrazatricyclo[12.3.1.12,5]nonadeca-1(18),2,4,14,16-pentaen-17-yl]carbamate (20 mg, 0.0254 mmol) and tert-butyl N-[(6R)-6-benzyloxy-12-[[4-(1-bicyclo[1.1.1]pentanyl)phenyl]methyl]-13-oxo-6,15-bis(trifluoromethyl)-19-oxa-3,4,12,18-tetrazatricyclo[12.3.1.12,5]nonadeca-1(17),2,4,14(18),15-pentaen-17-yl]carbamate (10 mg, 0.01273 mmol) obtained in part 1 was stirred in TFA (1 mL) at room temperature for 16 h and then the solvent was evaporated. The residue was dissolved in methanol and filtered then purified by preparative HPLC to obtain as a white solid, (6R)-17-amino-12-[[4-(1-bicyclo[1.1.1]pentanyl)phenyl]methyl]-6-hydroxy-6,15-bis(trifluoromethyl)-19-oxa-3,4,12,18-tetrazatricyclo[12.3.1.12,5]nonadeca-1(18),2,4,14,16-pentaen-13-one (7 mg, 46%). ESI-MS m/z calc. 595.2018, found 596.21 (M+1)⁺; Retention time: 2.14 minutes (LC Method A).

### Example 162: Preparation of 17-amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (enantiomer 1) (Compound 196) and 17-amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (Enantiomer 2) (Compound 197)

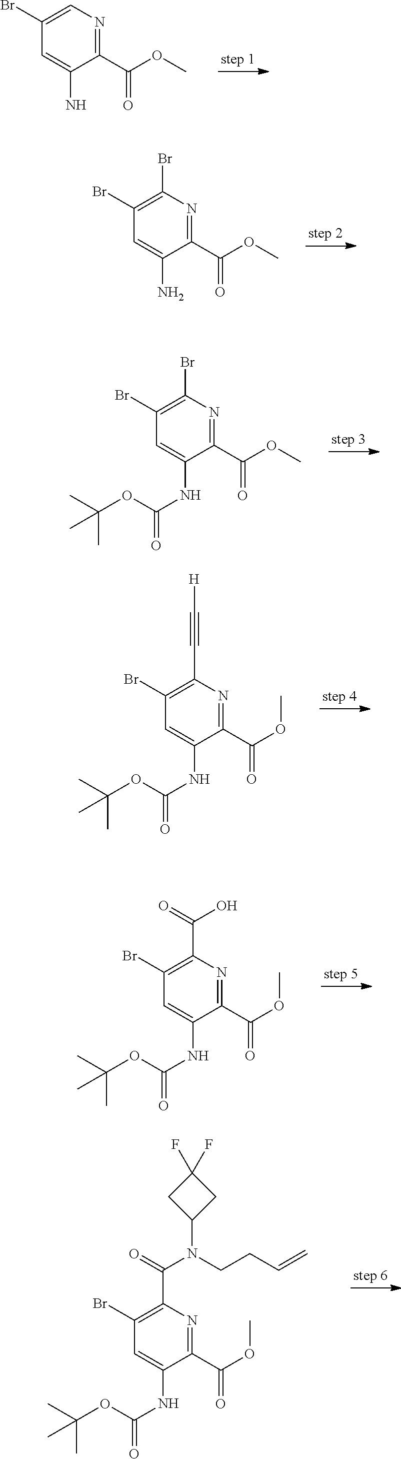

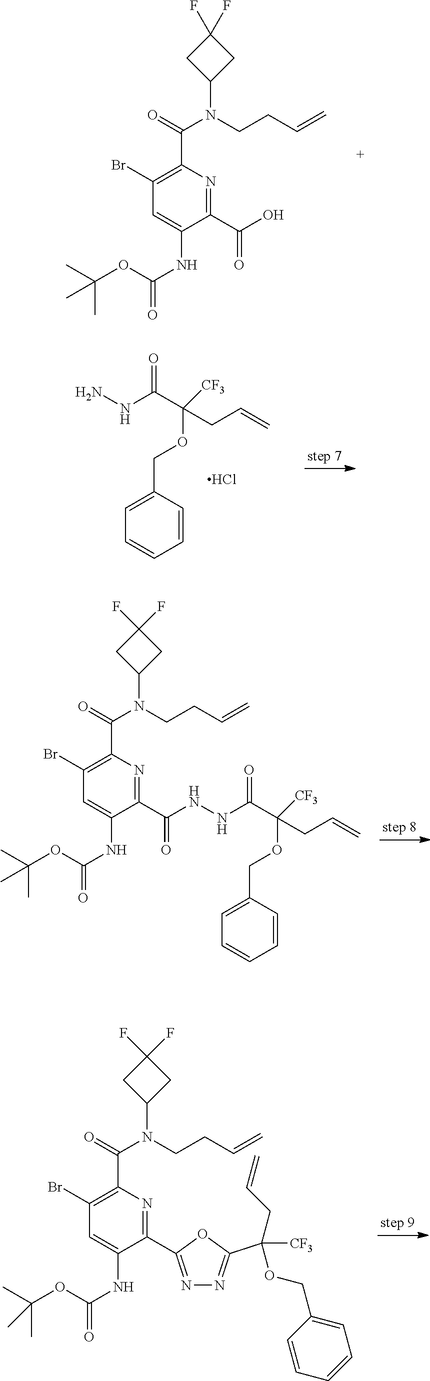

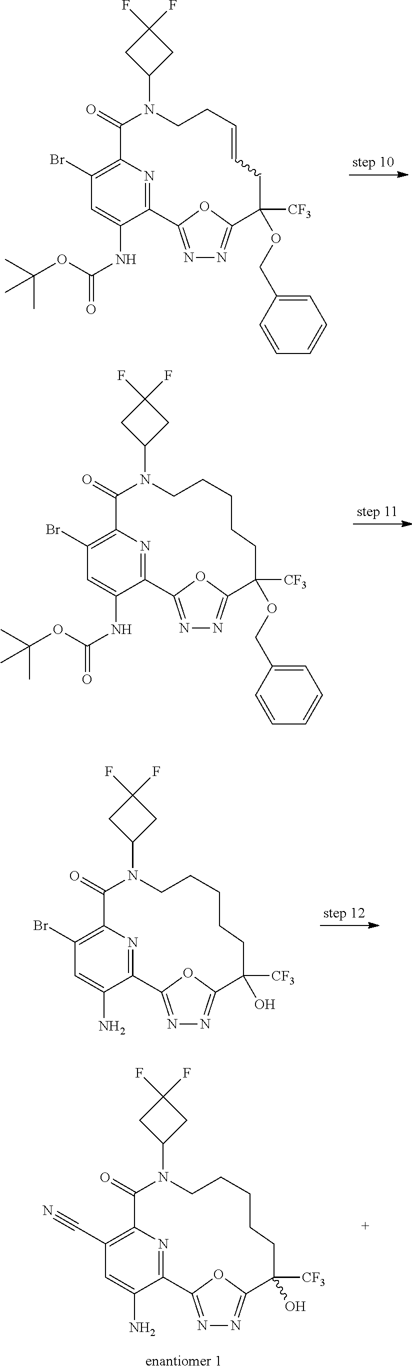 [SMILES: CC(C)(C)OC(=O)Nc1cc(Br)c2nc1C1=NN=C(O1)C(OCc1ccccc1)(C(F)(F)F)CC=CCCN(C1CC(F)(F)C1)C2=O ; CC(C)(C)OC(=O)Nc1cc(Br)c2nc1C1=NN=C(O1)C(OCc1ccccc1)(C(F)(F)F)CCCCCN(C1CC(F)(F)C1)C2=O ; Nc1cc(Br)c2nc1C1=NN=C(O1)C(O)(C(F)(F)F)CCCCCN(C1CC(F)(F)C1)C2=O ; N#Cc1cc(N)c2nc1C(=O)N(C1CC(F)(F)C1)CCCCCC(O)(C(F)(F)F)C1=NN=C2O1]

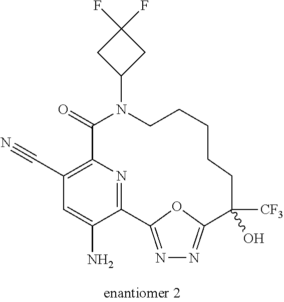 [SMILES: N#Cc1cc(N)c2nc1C(=O)N(C1CC(F)(F)C1)CCCCCC(O)(C(F)(F)F)C1=NN=C2O1]

### Step 1: Methyl 3-amino-5,6-dibromo-pyridine-2-carboxylate

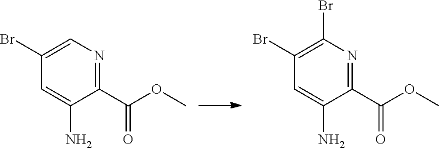

To a solution of methyl 3-amino-5-bromo-pyridine-2-carboxylate (3.15 g, 13.63 mmol) in acetonitrile (272.6 mL) was added 1-bromopyrrolidine-2,5-dione (2.668 g, 14.99 mmol) and the mixture was stirred at room temperature overnight. Concentrated the reaction mixture and recrystallized the product from hot toluene to afford as a pale orange solid, methyl 3-amino-5,6-dibromo-pyridine-2-carboxylate (4.1 g, 97%). ESI-MS m/z calc. 307.8796, found 310.86 (M+1)⁺; Retention time: 0.49 minutes (LC Method S).

### Step 2: Methyl 5,6-dibromo-3-(tert-butoxycarbonylamino)pyridine-2-carboxylate

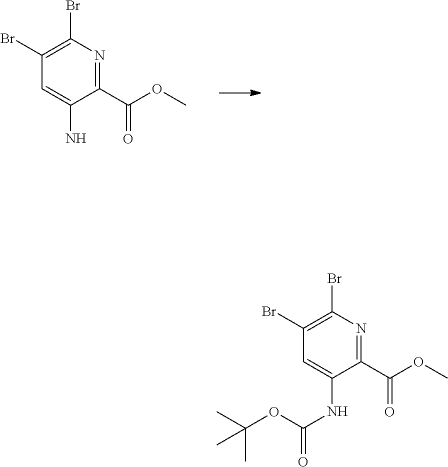 [SMILES: COC(=O)c1nc(Br)c(Br)cc1N ; COC(=O)c1nc(Br)c(Br)cc1NC(=O)OC(C)(C)C]

Methyl 5,6-dibromo-3-(tert-butoxycarbonylamino)pyridine-2-carboxylate was prepared from methyl 3-amino-5,6-dibromo-pyridine-2-carboxylate using a procedure analogous to step 1 of Method 11.

### Step 3: Methyl 5-bromo-3-(tert-butoxycarbonylamino)-6-ethynyl-pyridine-2-carboxylate

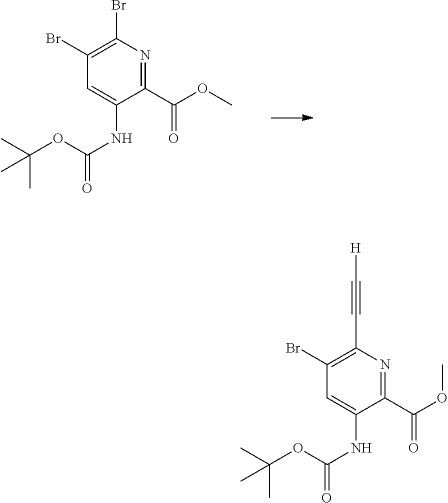 [SMILES: COC(=O)c1nc(Br)c(Br)cc1NC(=O)OC(C)(C)C ; C#Cc1nc(C(=O)OC)c(NC(=O)OC(C)(C)C)cc1Br]

To a microwave vial was added methyl 5,6-dibromo-3-(tert-butoxycarbonylamino)pyridine-2-carboxylate (500 mg, 1.219 mmol), PdCl₂(PPh₃)₂ (42.78 mg, 0.06095 mmol) and CuI (23.22 mg, 0.1219 mmol). Sealed the vial and purged with nitrogen. A solution of ethynyl(trimethyl)silane (119.7 mg, 1.219 mmol) and Et₃N (3.398 mL, 24.38 mmol) in dioxane (4.064 mL) was added. The mixture was stirred at room temperature for 16 h. The mixture was filtered through Celite, concentrated, and extracted with ethyl acetate (2×20 mL). The combined organic layers were washed with brine, dried over sodium sulfate, filtered and concentrated. The residue was dissolved in THF (2.5 mL), cooled to 0° C. and tetrabutylammonium fluoride (1.1 mL of 1 M, 1.1 mmol) as a solution in THF was added. The ice-bath was removed, and the mixture stirred for 10 min. The mixture was concentrated and purified by silica gel chromatography using a gradient from 0% to 20% ethyl acetate in hexanes to afford as a white solid, methyl 5-bromo-3-(tert-butoxycarbonylamino)-6-ethynyl-pyridine-2-carboxylate (130 mg, 30%): ¹H NMR (400 MHz, DMSO-d₆) δ 10.06 (s, 1H), 8.77 (s, 1H), 4.67 (s, 1H), 3.87 (s, 3H), 1.49 (s, 9H) ppm. ESI-MS m/z calc. 354.0215, found 355.03 (M+1)⁺; Retention time: 0.54 minutes (LC Method R).

### Step 4: 3-Bromo-5-(tert-butoxycarbonylamino)-6-methoxycarbonyl-pyridine-2-carboxylic Acid

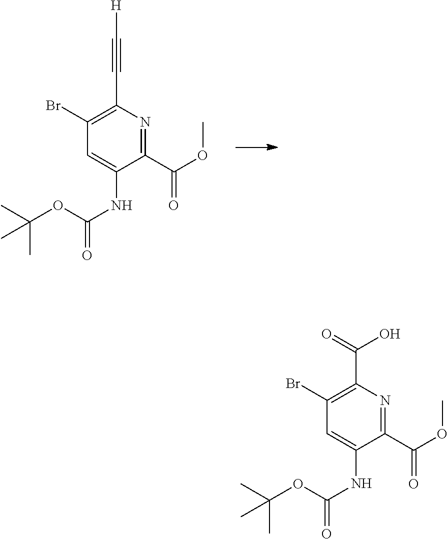 [SMILES: C#Cc1nc(C(=O)OC)c(NC(=O)OC(C)(C)C)cc1Br ; COC(=O)c1nc(C(=O)O)c(Br)cc1NC(=O)OC(C)(C)C]

To a round bottom flask was added methyl 5-bromo-3-(tert-butoxycarbonylamino)-6-ethynyl-pyridine-2-carboxylate (131 mg, 0.3688 mmol), potassium peroxymonosulfate (225.6 mg, 0.3688 mmol), NaHCO₃ (77.45 mg, 0.9220 mmol) and ruthenium on carbon (7.455 mg, 0.0037 mmol, 5% w/w). Added a 1:1 mixture of MeCN (1.048 mL) and water (1.048 mL) to the reaction mixture and stirred at room temperature for 3 h. Then more potassium peroxymonosulfate (225.6 mg, 0.3688 mmol) was added and the mixture stirred for another 2 hours. The mixture was filtered through Celite washing with ethyl acetate. The filtrate was diluted with citric acid (20 mL) and extracted with ethyl acetate (2×20 mL). The combined organic layers were washed with brine, dried (Na₂SO₄), filtered and concentrated to provide 3-bromo-5-(tert-butoxycarbonylamino)-6-methoxycarbonyl-pyridine-2-carboxylic acid (138.4 mg, quant.): ESI-MS m/z calc. 374.01135, found 376.94 (M+1)⁺; Retention time: 0.37 minutes (LC Method R).

### Step 5: Methyl 5-Bromo-6-[but-3-enyl-(3,3-difluorocyclobutyl)carbamoyl]-3-(tert-butoxycarbonylamino)pyridine-2-carboxylate

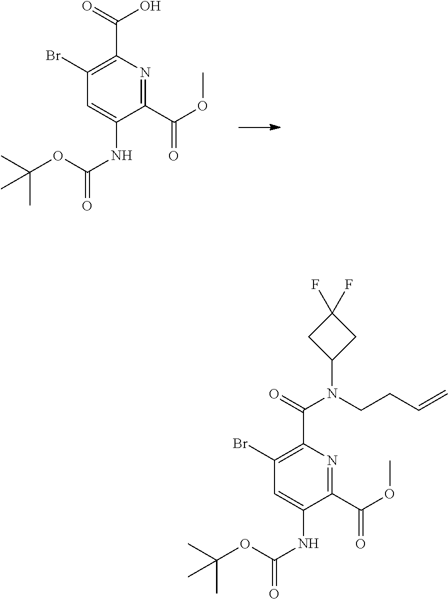 [SMILES: COC(=O)c1nc(C(=O)O)c(Br)cc1NC(=O)OC(C)(C)C ; C=CCCN(C(=O)c1nc(C(=O)OC)c(NC(=O)OC(C)(C)C)cc1Br)C1CC(F)(F)C1]

To a solution of 3-bromo-5-(tert-butoxycarbonylamino)-6-methoxycarbonyl-pyridine-2-carboxylic acid (130 mg, 0.3465 mmol) In DMF (1.494 mL) was added N-but-3-enyl-3,3-difluorocyclobutanamine (117.6 mg, 0.4159 mmol) and DIEA (361.1 μL, 2.073 mmol) followed by HATU (262.6 mg, 0.6906 mmol). The mixture was stirred at room temperature for 15 min. Added water and extracted with ethyl acetate (2×20 mL). Combined organic layers were washed with brined, dried over Na₂SO₄, filtered and concentrated. Purified by silica gel chromatography using 0% to 30% ethyl acetate in hexanes to afford as white solid, methyl 5-bromo-6-[but-3-enyl-(3,3-difluorocyclobutyl)carbamoyl]-3-(tert-butoxycarbonylamino)pyridine-2-carboxylate (96 mg, 53%). ESI-MS m/z calc. 517.1024, found 519.07 (M+1)⁺; Retention time: 0.61 minutes (LC Method R).

### Step 6: 5-Bromo-6-[but-3-enyl-(3,3-difluorocyclobutyl)carbamoyl]-3-(tert-butoxycarbonylamino)pyridine-2-carboxylic Acid

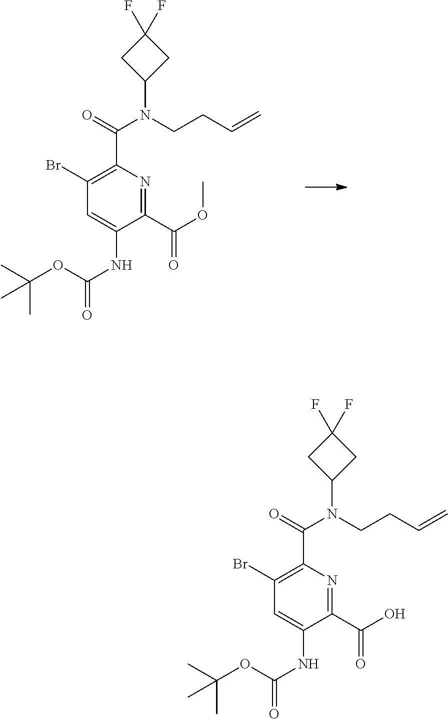 [SMILES: C=CCCN(C(=O)c1nc(C(=O)OC)c(NC(=O)OC(C)(C)C)cc1Br)C1CC(F)(F)C1 ; C=CCCN(C(=O)c1nc(C(=O)O)c(NC(=O)OC(C)(C)C)cc1Br)C1CC(F)(F)C1]

To a solution of methyl 5-bromo-6-[but-3-enyl-(3,3-difluorocyclobutyl)carbamoyl]-3-(tert-butoxycarbonylamino)pyridine-2-carboxylate (96 mg, 0.19 mmol) in THF (960 μL) were added methanol (960 μL) and water (480 μL). Lithium hydroxide (84.94 mg, 3.547 mmol) was added and the mixture was heated at to 60° C. for 1 h. THF and methanol were removed under reduced pressure. 1 M HCl solution was added until acidic, then the aqueous layer was extracted with ethyl acetate (3×75 mL). The organic phases were combined, washed with brine (75 mL), dried on anhydrous sodium sulfate, filtered and concentrated under reduced pressure to afford as a white solid 5-bromo-6-[but-3-enyl-(3,3-difluorocyclobutyl)carbamoyl]-3-(tert-butoxycarbonylamino)pyridine-2-carboxylic acid (93 mg, quant.): ESI-MS m/z calc. 503.08673, found 504.12 (M+1)⁺; Retention time: 0.56 minutes (LC Method R).

### Step 7: tert-Butyl N-[2-[[[2-Benzyloxy-2-(trifluoromethyl)pent-4-enoyl]amino]carbamoyl]-5-bromo-6-[but-3-enyl-(3,3-difluorocyclobutyl)carbamoyl]-3-pyridyl]carbamate

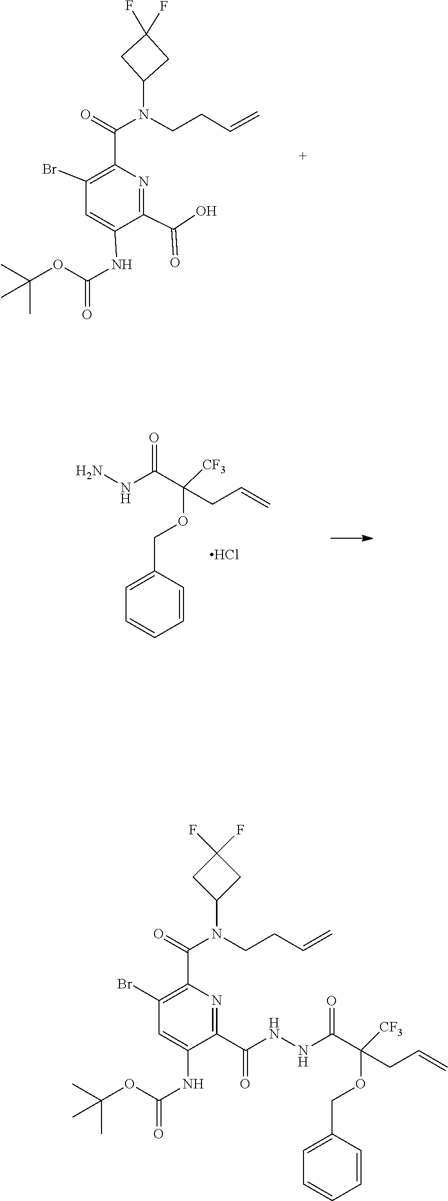 [SMILES: C=CCCN(C(=O)c1nc(C(=O)O)c(NC(=O)OC(C)(C)C)cc1Br)C1CC(F)(F)C1 ; C=CCC(OCc1ccccc1)(C(=O)NN)C(F)(F)F ; Cl ; C=CCCN(C(=O)c1nc(C(=O)NNC(=O)C(CC=C)(OCc2ccccc2)C(F)(F)F)c(NC(=O)OC(C)(C)C)cc1Br)C1CC(F)(F)C1]

To a solution of 5-bromo-6-[but-3-enyl-(3,3-difluorocyclobutyl)carbamoyl]-3-(tert-butoxycarbonylamino)pyridine-2-carboxylic acid (93 mg, 0.1844 mmol) in DMF (1.19 mL) was added 2-benzyloxy-2-(trifluoromethyl)pent-4-enehydrazide (hydrochloride salt) (60.72 mg, 0.187 mmol), DIEA (97.55 μL, 0.56 mmol), followed by HATU (84.14 mg, 0.2213 mmol). The reaction mixture was stirred at room temperature for 15 minutes. The reaction was extracted with ethyl acetate (3×20 mL). The organic layers were washed with brine (20 mL), dried over sodium sulfate, and evaporated. The residue was then purified by silica gel chromatography (12 gram column) using 0% to 70% ethyl acetate in hexanes to afford tert-butyl N-[2-[[[2-benzyloxy-2-(trifluoromethyl)pent-4-enoyl]amino]carbamoyl]-5-bromo-6-[but-3-enyl-(3,3-difluorocyclobutyl)carbamoyl]-3-pyridyl]carbamate (105 mg, 74%). ESI-MS m/z calc. 773.18475, found 774.25 (M+1)⁺; Retention time: 0.8 minutes (LC Method R).

### Step 8: tert-Butyl N-[2-[5-[1-benzyloxy-1-(trifluoromethyl)but-3-enyl]-1,3,4-oxadiazol-2-yl]-5-bromo-6-[but-3-enyl-(3,3-difluorocyclobutyl)carbamoyl]-3-pyridyl]carbamate

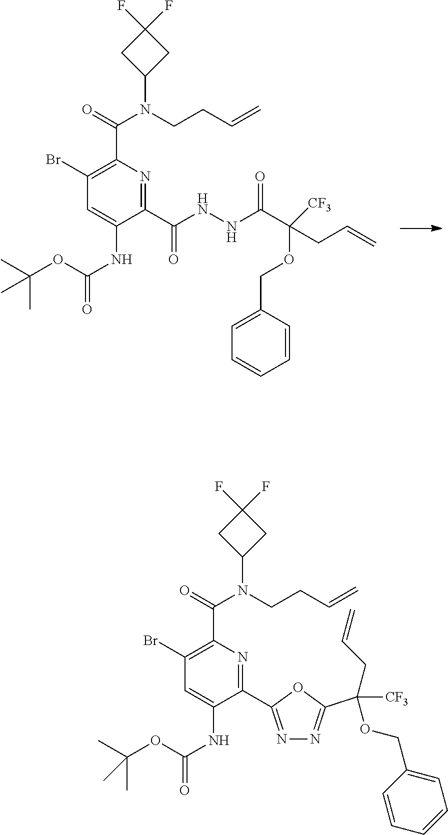

A solution of tert-butyl N-[2-[[[2-benzyloxy-2-(trifluoromethyl)pent-4-enoyl]amino]carbamoyl]-5-bromo-6-[but-3-enyl-(3,3-difluorocyclobutyl)carbamoyl]-3-pyridyl]carbamate (105 mg, 0.1356 mmol) and DIEA (82.08 μL, 0.4712 mmol) in acetonitrile (2.4 mL) was heated to 60° C., then p-toluenesulfonyl chloride (31.02 mg, 0.1627 mmol) was added. The resulting mixture was heated at 60° C. for 30 minutes. After cooling, saturated sodium bicarbonate (25 mL) was added and the mixture extracted with ethyl acetate (3×25 mL). The organics were separated, dried over sodium sulfate, and evaporated. The crude material was then purified by silica gel chromatography (12 gram column) using 0% to 30% ethyl acetate in hexanes to afford as a white solid, tert-butyl N-[2-[5-[1-benzyloxy-1-(trifluoromethyl)but-3-enyl]-1,3,4-oxadiazol-2-yl]-5-bromo-6-[but-3-enyl-(3,3-difluorocyclobutyl)carbamoyl]-3-pyridyl]carbamate (75 mg, 73%): ESI-MS m/z calc. 755.1742, found 756.21 (M+1)⁺; Retention time: 0.72 minutes (LC Method T).

### Step 9: tert-Butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,8,14(18),15-hexaen-17-yl]carbamate (E/Z Mixture)

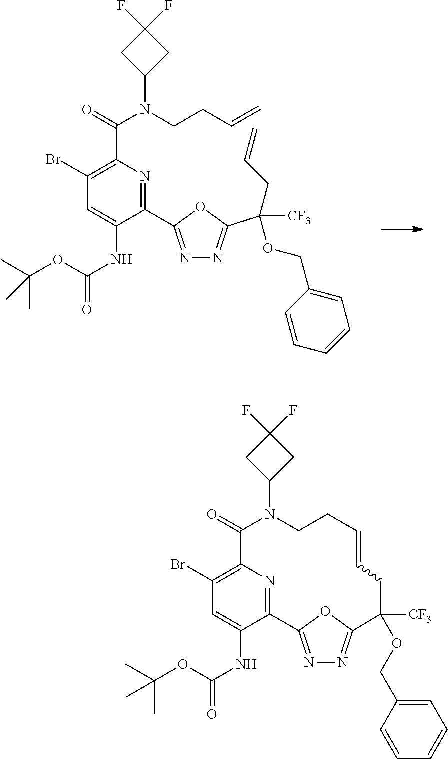 [SMILES: C=CCCN(C(=O)c1nc(-c2nnc(C(CC=C)(OCc3ccccc3)C(F)(F)F)o2)c(NC(=O)OC(C)(C)C)cc1Br)C1CC(F)(F)C1 ; CC(C)(C)OC(=O)Nc1cc(Br)c2nc1C1=NN=C(O1)C(OCc1ccccc1)(C(F)(F)F)CC=CCCN(C1CC(F)(F)C1)C2=O]

To a nitrogen purged flask was added tert-butyl N-[2-[5-[1-benzyloxy-1-(trifluoromethyl)but-3-enyl]-1,3,4-oxadiazol-2-yl]-5-bromo-6-[but-3-enyl-(3,3-difluorocyclobutyl)carbamoyl]-3-pyridyl]carbamate (75 mg, 0.09913 mmol) in 1,2-dichloroethane (17.36 mL) and it was heated to 60° C. Then added via syringe dichloro[1,3-bis(2,4,6-trimethylphenyl)-2-imidazolidinylidene][[5-[(dimethylamino)sulfonyl]-2-(1-methylethoxy-O)phenyl]methylene-C]ruthenium(II) (18.18 mg, 0.02478 mmol) in 1,2-dichloroethane (2 mL), and the solution heated to 60° C. and let stir for 45 min. The reaction mixture was concentrated and purified by silica gel chromatography using 0% to 30% ethyl acetate in hexanes to afford as a white solid, tert-butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (65 mg, 90%): ESI-MS m/z calc. 727.1429, found 728.2 (M+1)⁺; Retention time: 0.67 minutes (LC Method T).

### Step 10: tert-Butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,14(18),15-pentaen-17-yl]carbamate

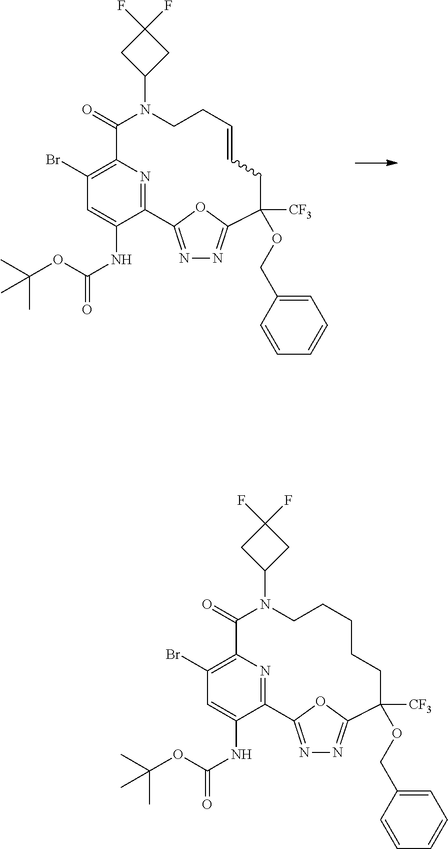 [SMILES: CC(C)(C)OC(=O)Nc1cc(Br)c2nc1C1=NN=C(O1)C(OCc1ccccc1)(C(F)(F)F)CC=CCCN(C1CC(F)(F)C1)C2=O ; CC(C)(C)OC(=O)Nc1cc(Br)c2nc1C1=NN=C(O1)C(OCc1ccccc1)(C(F)(F)F)CCCCCN(C1CC(F)(F)C1)C2=O]

To a solution of tert-butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,8,14(18),15-hexaen-17-yl]carbamate (E/Z mixture) (60 mg, 0.08236 mmol) in ethyl acetate (4.154 mL) under nitrogen atmosphere was added rhodium on alumina (16.63 mg of 5% w/w, 0.00808 mmol). The reaction was put under hydrogen atmosphere and stirred at room temperature for 4 hours, then filtered on Celite and concentrated. The residue was purified by silica gel chromatography using 0% to 20% ethyl acetate in hexanes to provide as a white solid, tert-butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,14(18),15-pentaen-17-yl]carbamate (40 mg, 66%): ¹H NMR (400 MHz, Chloroform-d) δ 9.77 (s, 1H), 9.27 (s, 1H), 7.34-7.24 (m, 5H), 4.94-4.73 (m, 2H), 4.38-4.19 (m, 1H), 3.22 (t, J=8.0 Hz, 2H), 3.00 (m, 4H), 2.55 (m, J=13.8, 8.2, 4.5 Hz, 1H), 2.21 (m, J=13.8, 5.6 Hz, 1H), 2.04 (s, 2H), 1.84 (m, J=12.6, 6.0, 5.4 Hz, 1H), 1.72 (m, J=9.3, 5.1 Hz, 1H), 1.62 (s, 1H), 1.56 (s, 10H) ppm. ESI-MS m/z calc. 729.1585, found 730.1 (M+1)⁺; Retention time: 0.66 minutes (LC Method T).

### Step 11: 17-Amino-15-bromo-12-(3,3-difluorocyclobutyl)-6-hydroxy-6-(trifluoromethyl)-19-oxa-3,4,12,18-tetrazatricyclo[12.3.1.12,5]nonadeca-1(17),2,4,14(18),15-pentaen-13-one

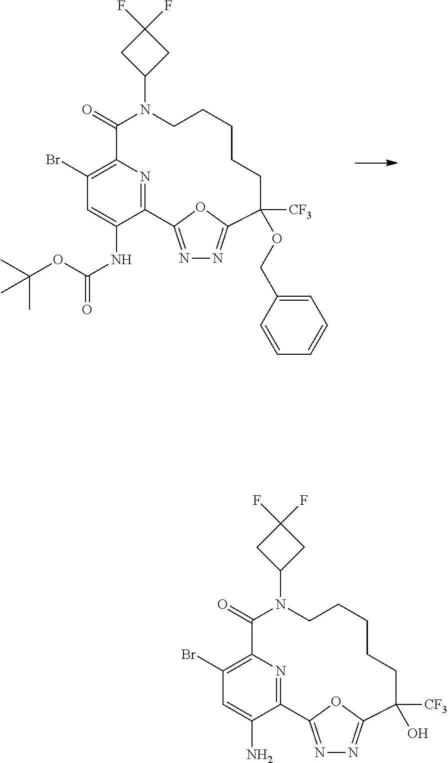 [SMILES: CC(C)(C)OC(=O)Nc1cc(Br)c2nc1C1=NN=C(O1)C(OCc1ccccc1)(C(F)(F)F)CCCCCN(C1CC(F)(F)C1)C2=O ; Nc1cc(Br)c2nc1C1=NN=C(O1)C(O)(C(F)(F)F)CCCCCN(C1CC(F)(F)C1)C2=O]

TFA (800 μL) was added to tert-butyl N-[6-benzyloxy-15-bromo-12-(3,3-difluorocyclobutyl)-13-oxo-6-(trifluoromethyl)-19-oxa-3,4,12,18-tetrazatricyclo[12.3.1.12,5]nonadeca-1(17),2,4,14(18),15-pentaen-17-yl]carbamate (42 mg, 0.05749 mmol) and stirred at room temperature overnight. After concentrating the mixture, the residue was dissolved in DCM and washed with saturated aqueous NaHCO₃, then brine, dried over Na₂SO₄, filtered and concentrated. Purification by silica gel chromatography using 0% to 40% ethyl acetate in hexanes afford as a white solid, 17-amino-15-bromo-12-(3,3-difluorocyclobutyl)-6-hydroxy-6-(trifluoromethyl)-19-oxa-3,4,12,18-tetrazatricyclo[12.3.1.12,5]nonadeca-1(17),2,4,14(18),15-pentaen-13-one (29 mg, 93%): ESI-MS m/z calc. 539.05914, found 540.0 (M+1)⁺; Retention time: 0.37 minutes (LC Method R).

### Step 12: 17-Amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (enantiomer 1) (Compound 196) and 17-amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (Enantiomer 2) (Compound 197)

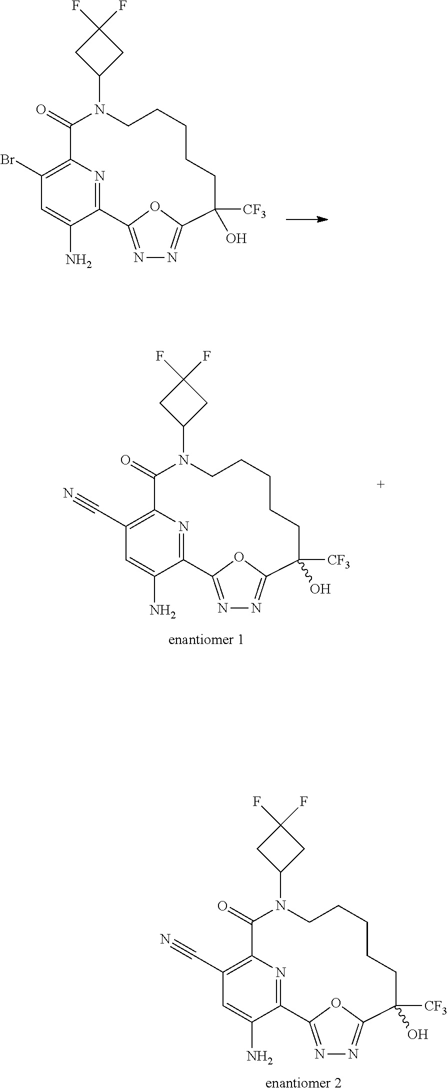 [SMILES: Nc1cc(Br)c2nc1-c1nnc(o1)C(O)(C(F)(F)F)CCCCCN(C1CC(F)(F)C1)C2=O ; N#Cc1cc(N)c2nc1C(=O)N(C1CC(F)(F)C1)CCCCCC(O)(C(F)(F)F)c1nnc-2o1 ; N#Cc1cc(N)c2nc1C(=O)N(C1CC(F)(F)C1)CCCCCC(O)(C(F)(F)F)c1nnc-2o1]

In a microwave vial, 17-amino-15-bromo-12-(3,3-difluorocyclobutyl)-6-hydroxy-6-(trifluoromethyl)-19-oxa-3,4,12,18-tetrazatricyclo[12.3.1.12,5]nonadeca-1(17),2,4,14(18),15-pentaen-13-one (10 mg, 0.01851 mmol) was dissolved in DMF (200 μL) and to the mixture was added dicyanozinc (3.252 mg, 0.02769 mmol) followed by tetrakis(triphenylphosphine)palladium(O) (1 mg, 0.87 μmol). The mixture was purged with nitrogen, and heated in microwave synthesizer for 48 h at 105° C. The mixture was cooled to room temperature filtered through Celite and extracted with ethyl acetate (3×15 mL). The combined organic layers were washed with brine, dried over Na₂SO₄, filtered and concentrated. Purification using a normal phase SFC-MS method with an IG column (250×21.2 mm, 5 mm particle size, sold by Chiral Technologies) using a dual gradient run from 5% to 15% of MeOH (+20 mM NH₃) in CO₂ over 14.5 minutes provided as the first eluent, 17-amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (enantiomer 1) (4 mg, 44%). ¹H NMR (400 MHz, Chloroform-d) δ 7.57 (s, 1H), 6.09 (s, 2H), 4.42 (m, 1H), 3.47 (m, J=15.3, 12.3, 3.6 Hz, 1H), 3.18 (m, J=14.8, 13.0, 5.3 Hz, 1H), 3.10-2.86 (m, 4H), 2.48 (m, J=13.9, 8.5, 4.8 Hz, 2H), 2.29 (m, J=15.0, 7.6, 4.8 Hz, 1H), 1.78 (q, J=6.4, 4.9 Hz, 3H), 1.34 (m, J=11.9, 9.5, 5.4 Hz, 2H) ppm. ESI-MS m/z calc. 486.1439, found 487.18 (M+1)⁺; Retention time: 1.44 minutes (LC Method A).

The second eluent provided 17-amino-12-(3,3-difluorocyclobutyl)-6-hydroxy-13-oxo-6-(trifluoromethyl)-19-oxa-3,4,12,18-tetrazatricyclo[12.3.1.12,5]nonadeca-1(18),2,4,14,16-pentaene-15-carbonitrile (enantiomer 2) (4 mg, 44%). ¹H NMR (400 MHz, Chloroform-d) δ 7.57 (s, 1H), 6.10 (s, 2H), 4.43 (m, J=8.7, 3.7 Hz, 1H), 3.57-3.37 (m, 1H), 3.29-3.12 (m, 1H), 3.02 (m, J=15.0, 6.9, 6.3, 4.1 Hz, 3H), 2.48 (m, J=13.9, 8.6, 5.0 Hz, 2H), 2.29 (m, J=10.3, 7.7, 3.8 Hz, 1H), 1.92-1.69 (m, 3H), 1.42-1.27 (m, 2H), 1.01-0.80 (m, 2H) ppm. ESI-MS m/z calc. 486.1439, found 487.18 (M+1)⁺; Retention time: 1.43 minutes (LC Method N).

### Method 14: Final Product Synthesis by Macrolactamization Route

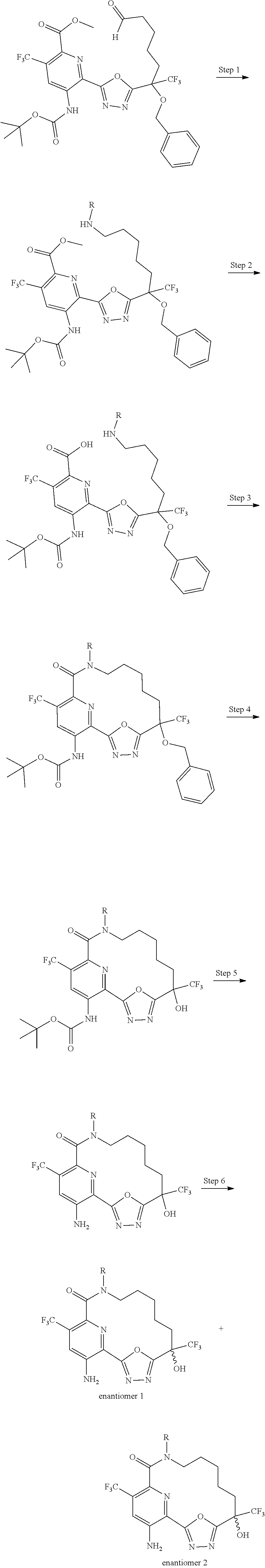

### Step 1: Reductive Amination

The appropriate aldehyde (for example, methyl 6-[5-[1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate, methyl 6-[5-[(1R)-1-benzyloxy-6-oxo-1-(trifluoromethyl)hexyl]-1,3,4-oxadiazol-2-yl]-5-(tert-butoxycarbonylamino)-3-(trifluoromethyl)pyridine-2-carboxylate, methyl 6-[5-[1-benzyloxy-5-oxo-1-(trifluoromethyl)pentyl]-1,3,4-oxadiazol-2-yl]-5-(tert-butoxycarbonylamino)-3-(trifluoromethyl)pyridine-2-carboxylate, methyl 5-(tert-butoxycarbonylamino)-6-[5-[2,2,2-trifluoro-1-[2-fluoro-5-(3-oxopropyl)phenyl]-1-hydroxy-ethyl]-1,3,4-oxadiazol-2-yl]-3-(trifluoromethyl)pyridine-2-carboxylate, or methyl 6-[5-[1-benzyloxy-2,2,2-trifluoro-1-(3-oxopropoxymethyl)ethyl]-1,3,4-oxadiazol-2-yl]-5-(tert-butoxycarbonylamino)-3-(trifluoromethyl)pyridine-2-carboxylate) (1 eq) was combined with isopropyl amine (˜5 equivalents) in dichloromethane or MeOH-acetic acid mixture (2 to 5 volume equivalents) and stirred for about 30 minutes at room temperature. Then reducing agent, sodium triacetoxyborohydride or sodium cyanoborohydride (˜5 equivalents) was added, and the reaction mixture was stirred for an additional 30 to 60 minutes at room temperature. The reaction progress was monitored by LCMS. After completion of the reaction, the reaction was diluted with ethyl acetate and washed with aqueous sodium bicarbonate solution and brine. The organic solution was dried over anhydrous sodium sulfate and concentrated under vacuum. The residue was purified by silica gel chromatography using ethyl acetate/hexanes or DCM/MeOH to give the desired secondary amines.

### Step 2: Saponification

Amino ester intermediate (1 equivalent) was dissolved in dioxane/water or MeOH/THF/water (5 to 10 volume equivalents) and LiOH or NaOH (˜5 equivalents) was added. The reaction mixture was stirred at room temperature for a few hours to overnight. The reaction progress was monitored by LCMS. After completion of the reaction, diluted with ethyl acetate and saturated aqueous ammonium chloride. The aqueous pH was adjusted to ˜5 with 1N HCl. The resulting two layers were separated, and the aqueous layer was extracted with ethyl acetate. The combined organic layers were washed with 1N HCl and brine, then concentrated under vacuum to furnish the desired acid.

### Step 3: Macrolactamization

To a solution of amino acid (1 equivalent) in DMF (300 to 500 volume equivalents) was added DIEA (˜5 equivalents) followed by HATU (˜1.5 equivalents). The reaction mixture was stirred at room temperature for a couple of hours while monitoring the reaction progress by LCMS. After completion of the reaction, the reaction mixture was diluted with ethyl acetate and water. The two layers were separated and the aqueous layer was extracted with ethyl acetate. The combined organic layers were washed with brine, dried over anhydrous sodium sulfate, filtered and concentrated under vacuum. The residue was purified by silica gel chromatography using ethyl acetate in hexanes which provided the desired macrocycles.

### Step 4: Hydrogenation

Debenzylation of the macrocycle (1 equivalent) was performed using 10% Pd/C (0.2 to 0.5 equivalents) in ethyl acetate, ethanol or methanol (10 to 50 volume equivalents) while stirring under a hydrogen balloon (1 atmosphere of gas) for 12 to 24 hours. The catalyst was filtered off through a pad of Celite. The filtrate was concentrated under vacuum to furnish the desired alcohol.

### Step 5: N-Boc Deprotection

The N-Boc protected macrocycle (1 equivalent) was dissolved in DCM or water (˜20 volume equivalents) and to the mixture was added TFA (˜20 equivalents) and the mixture was stirred at room temperature for a couple of hours while monitoring the reaction progress by LCMS. After the starting material was consumed, the solvents were evaporated and the residue was dissolved in DCM then washed with saturated aqueous NaHCO₃ and brine then dried over Na₂SO₄, filtered and concentrated to give the desired product.

### Step 6: Chiral Separation

Racemic compounds were purified by chiral SFC using a suitable chiral column and mobile phase as indicated in Table 6 giving as the first eluting enantiomer, enantiomer 1 and the later eluting enantiomer, enantiomer 2.

### Representative Synthesis of Final Compounds by Method 14 (Macrolactamization Route)

### Example 163: Preparation of 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (enantiomer 1) (Compound 198) and 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (Enantiomer 2) (Compound 199)

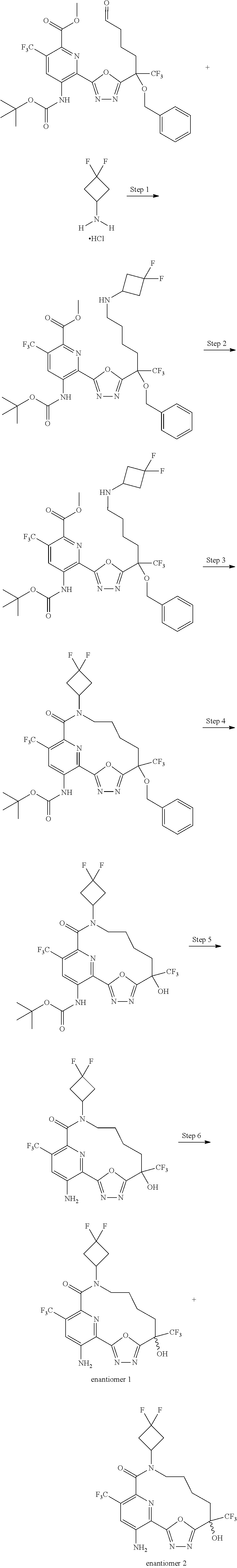

### Step 1: Methyl 6-[5-[1-Benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylate

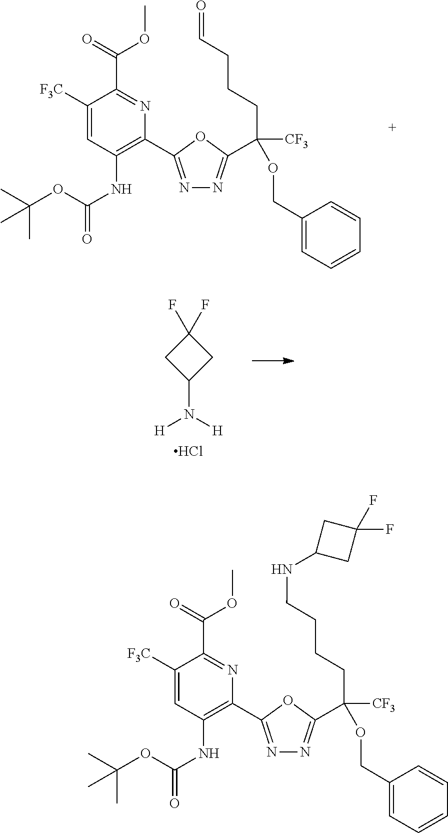 [SMILES: COC(=O)c1nc(-c2nnc(C(CCCC=O)(OCc3ccccc3)C(F)(F)F)o2)c(NC(=O)OC(C)(C)C)cc1C(F)(F)F ; NC1CC(F)(F)C1 ; Cl ; COC(=O)c1nc(-c2nnc(C(CCCCNC3CC(F)(F)C3)(OCc3ccccc3)C(F)(F)F)o2)c(NC(=O)OC(C)(C)C)cc1C(F)(F)F]

Into a solution of 3,3-difluorocyclobutanamine (hydrochloride salt) (832 mg, 5.6795 mmol) in a solvent mixture of methanol (4.5 mL) and acetic acid (0.5 mL) was added sodium cyanotrihydroborate (140 mg, 2.2278 mmol) at 0° C. A solution of methyl 6-[5-[1-benzyloxy-5-oxo-1-(trifluoromethyl)pentyl]-1,3,4-oxadiazol-2-yl]-5-(tert-butoxycarbonylamino)-3-(trifluoromethyl)pyridine-2-carboxylate (382 mg, 0.5613 mmol) in methanol (1.8 mL) and acetic acid (0.2 mL) was added drop-wise at 0° C. The reaction was stirred at the same temperature for 10 minutes, then raised to room temperature and stirred for 1 hour. The reaction was diluted with ethyl acetate (100 mL) and washed with saturated aqueous sodium bicarbonate aqueous solution (30 mL) and brine (20 mL). The organic solution was dried over anhydrous sodium sulfate and concentrated under vacuum. The residue was purified by silica gel chromatography using 0 to 70% ethyl acetate in hexanes to furnish methyl 6-[5-[1-benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylate (347 mg, 83%) as a yellow gel. ESI-MS m/z calc. 737.246, found 738.5 (M+1)⁺; Retention time: 3.69 minutes (LC Method G).

### Step 2: 6-[5-[1-Benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylic Acid

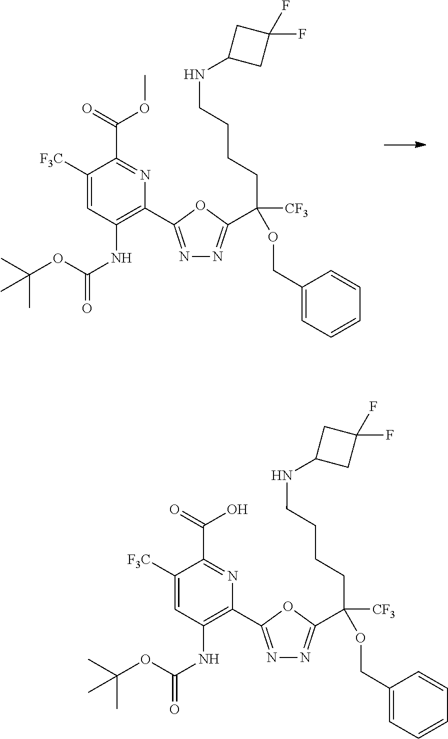 [SMILES: COC(=O)c1nc(-c2nnc(C(CCCCNC3CC(F)(F)C3)(OCc3ccccc3)C(F)(F)F)o2)c(NC(=O)OC(C)(C)C)cc1C(F)(F)F ; CC(C)(C)OC(=O)Nc1cc(C(F)(F)F)c(C(=O)O)nc1-c1nnc(C(CCCCNC2CC(F)(F)C2)(OCc2ccccc2)C(F)(F)F)o1]

Into a solution of methyl 6-[5-[1-benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylate (347 mg, 0.4657 mmol) in dioxane (3 mL) and water (3 mL) was added LiOH (69 mg, 2.8812 mmol). The reaction mixture was stirred at room temperature for 3 hours. The reaction was diluted with ethyl acetate (50 mL) and saturated aqueous ammonium chloride (30 mL). The aqueous pH was adjusted to 5 with 1N HCl. Two layers were separated and the aqueous layer was extracted with ethyl acetate (2×50 mL). The combined organic layers were washed with 1N HCl (2×20 mL) and brine (50 mL), then concentrated under vacuum to furnish 6-[5-[1-benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylic acid (362 mg, 91%) as a clear gel. ESI-MS m/z calc. 723.2303, found 724.5 (M+1)⁺; Retention time: 3.24 minutes (LC Method G).

### Step 3: tert-Butyl N-[6-benzyloxy-11-(3,3-difluorocyclobutyl)-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate

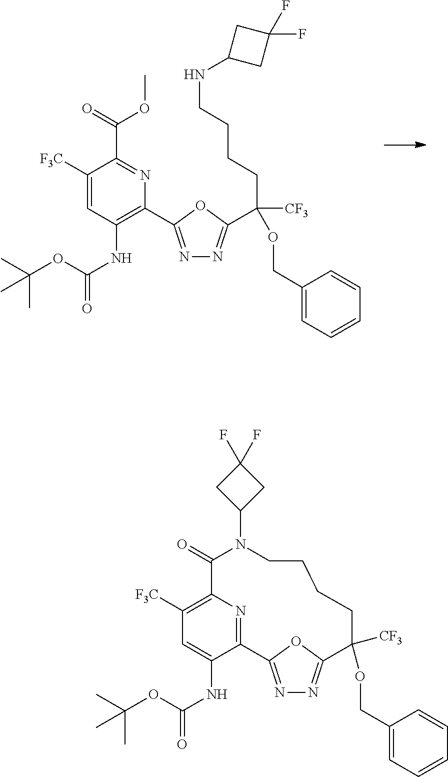 [SMILES: COC(=O)c1nc(-c2nnc(C(CCCCNC3CC(F)(F)C3)(OCc3ccccc3)C(F)(F)F)o2)c(NC(=O)OC(C)(C)C)cc1C(F)(F)F ; CC(C)(C)OC(=O)Nc1cc(C(F)(F)F)c2nc1C1=NN=C(O1)C(OCc1ccccc1)(C(F)(F)F)CCCCN(C1CC(F)(F)C1)C2=O]

A round bottom flask was charged with a solution of 6-[5-[1-benzyloxy-5-[(3,3-difluorocyclobutyl)amino]-1-(trifluoromethyl)pentyl]-1,3,4-oxadiazol-2-yl]-5-(tert-butoxycarbonylamino)-3-(trifluoromethyl)pyridine-2-carboxylic acid (342 mg, 0.4005 mmol) in DMF (150 mL). DIEA (259.70 mg, 0.35 mL, 2.0094 mmol) was added to the reaction mixture, followed by HATU (229 mg, 0.6023 mmol). The reaction mixture was stirred at room temperature for 1 hour. The reaction was diluted with ethyl acetate (150 mL) and water (150 mL). Two layers were separated and the aqueous layer was extracted with ethyl acetate (2×150 mL). The combined organic layers were washed with brine (3×100 mL), dried over anhydrous sodium sulfate, filtered and concentrated under vacuum. The residue was purified by silica gel chromatography using a gradient from 0% to 20% ethyl acetate in hexane to furnish tert-butyl N-[6-benzyloxy-11-(3,3-difluorocyclobutyl)-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate (93 mg, 33%) as a white foam. ESI-MS m/z calc. 705.2197, found 706.3 (M+1)⁺; Retention time: 4.09 minutes (LC Method G).

### Step 4: tert-Butyl N-[11-(3,3-difluorocyclobutyl)-6-hydroxy-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate

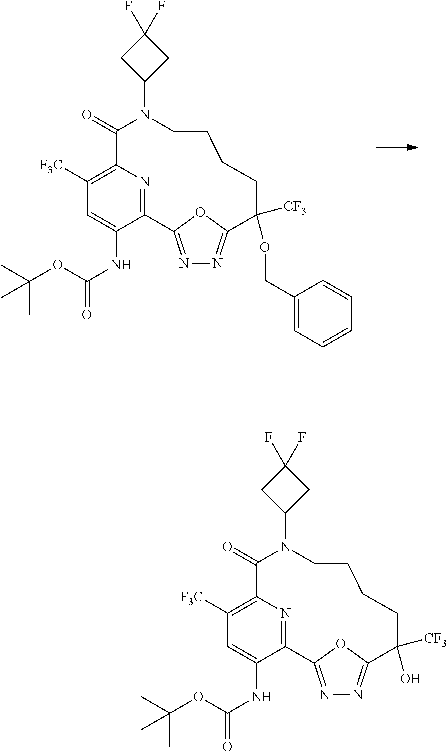 [SMILES: CC(C)(C)OC(=O)Nc1cc(C(F)(F)F)c2nc1C1=NN=C(O1)C(OCc1ccccc1)(C(F)(F)F)CCCCN(C1CC(F)(F)C1)C2=O ; CC(C)(C)OC(=O)Nc1cc(C(F)(F)F)c2nc1C1=NN=C(O1)C(O)(C(F)(F)F)CCCCN(C1CC(F)(F)C1)C2=O]

Into a solution of tert-butyl N-[6-benzyloxy-11-(3,3-difluorocyclobutyl)-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate (93 mg, 0.1318 mmol) in ethyl acetate (10 mL) was added 10% Pd/C (70 mg, 10% w/w, 0.0658 mmol). The reaction was stirred under 1 atmosphere of hydrogen gas for 24 hours. The catalyst was filtered off through a pad of Celite. The filtrate was concentrated under vacuum to furnish tert-butyl N-[11-(3,3-difluorocyclobutyl)-6-hydroxy-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate (82 mg, quant.) as a clear gel. ESI-MS m/z calc. 615.1728, found 616.4 (M+1)⁺; Retention time: 3.61 minutes (LC Method G).

### Step 5: 16-Amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one

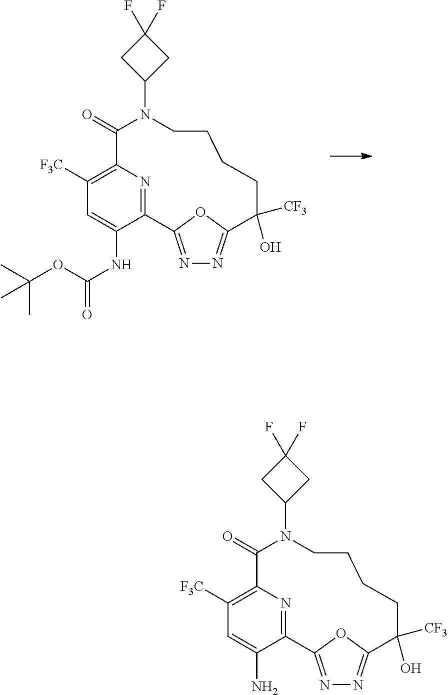 [SMILES: CC(C)(C)OC(=O)Nc1cc(C(F)(F)F)c2nc1C1=NN=C(O1)C(O)(C(F)(F)F)CCCCN(C1CC(F)(F)C1)C2=O ; Nc1cc(C(F)(F)F)c2nc1C1=NN=C(O1)C(O)(C(F)(F)F)CCCCN(C1CC(F)(F)C1)C2=O]

Into a solution of tert-butyl N-[11-(3,3-difluorocyclobutyl)-6-hydroxy-12-oxo-6,14-bis(trifluoromethyl)-18-oxa-3,4,11,17-tetrazatricyclo[11.3.1.12,5]octadeca-1(16),2,4,13(17),14-pentaen-16-yl]carbamate (82 mg, 0.1332 mmol) in DCM (5 mL) was added TFA (3.7 g, 2.5 mL, 32.45 mmol) at 0° C. The reaction was stirred at room temperature for 1 hour. The reaction was diluted with ethyl acetate (50 mL) and saturated sodium bicarbonate (50 mL) bringing the aqueous phase to pH=7. Two layers were separated and the organic layer was dried over anhydrous sodium sulfate and concentrated under vacuum. The residue was purified by silica gel chromatography using a gradient from 0% to 50% ethyl acetate in hexane to furnish 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (27.9 mg, 39%) as a yellow solid: ¹H NMR (500 MHz, DMSO-d₆) δ 7.76 (s, 1H), 7.59 (s, 1H), 7.06 (s, 2H), 4.22-4.11 (m, 1H), 3.61-3.39 (m, 1H), 3.24-3.13 (m, 1H), 3.12-2.84 (m, 4H), 2.40-2.24 (m, 2H), 2.19-2.07 (m, 1H), 2.05-1.94 (m, 1H), 1.58-1.22 (m, 2H) ppm. ESI-MS m/z calc. 515.1204, found 516.1 (M+1)⁺; Retention time: 2.48 minutes (LC Method H).

### Step 6: 16-Amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (enantiomer 1) (Compound 198) and 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (Enantiomer 2) (Compound 199)

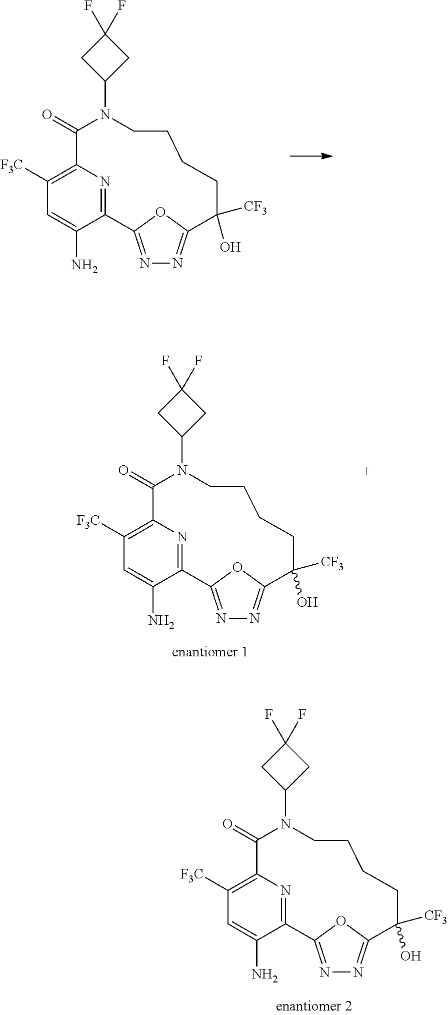 [SMILES: Nc1cc(C(F)(F)F)c2nc1C1=NN=C(O1)C(O)(C(F)(F)F)CCCCN(C1CC(F)(F)C1)C2=O ; Nc1cc(C(F)(F)F)c2nc1C1=NN=C(O1)C(O)(C(F)(F)F)CCCCN(C1CC(F)(F)C1)C2=O ; Nc1cc(C(F)(F)F)c2nc1C1=NN=C(O1)C(O)(C(F)(F)F)CCCCN(C1CC(F)(F)C1)C2=O]

16-Amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (28 mg, 0.05433 mmol) was dissolved into acetonitrile at 6 mg/mL and purified by preparative SFC with 200 μL injections through a preparative SFC eluting a gradient of 5 mM NH₃ in methanol to CO₂ (10% to 60% over 10 min at 60 mL/min) though a 21.2×250 mm AD column, 5 m particle size, which gave as the first product to elute and a white solid, 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (enantiomer 1) (7 mg, 25%)¹H NMR (400 MHz, methanol-d4) δ 7.71 (s, 1H), 4.27 (m, J=8.5, 3.4 Hz, 1H), 3.59 (s, 1H), 3.25 (s, 1H), 3.11-3.02 (m, 1H), 2.96 (m, J=15.0, 7.5 Hz, 2H), 2.48 (d, J=46.1 Hz, 2H), 2.16 (s, 2H), 1.57 (d, J=20.4 Hz, 1H), 1.33 (s, 2H) ppm; ¹⁹F NMR (376 MHz, methanol-d4) δ−61.55, −81.07, −84.99 (d, J=199.0 Hz), −102.39 (d, J=198.5 Hz) ppm. ESI-MS m/z calc. 515.12036, found 516.1 (M+1)⁺; Retention time: 1.24 minutes (LC Method Q).

Continued elution provided 16-amino-11-(3,3-difluorocyclobutyl)-6-hydroxy-6,14-bis(trifluoromethyl)-18-oxa-3,4,11,17-tetrazatricyclo[11.3.1.12,5]octadeca-1(17),2,4,13,15-pentaen-12-one (enantiomer 2) (7.5 mg, 27%)¹H NMR (400 MHz, methanol-d4) δ 7.71 (s, 1H), 4.37-4.21 (m, 1H), 3.59 (s, 1H), 3.25 (s, 1H), 3.00 (m, 3H), 2.53 (s, 1H), 2.42 (s, 1H), 2.16 (s, 2H), 1.57 (d, J=22.8 Hz, 1H), 1.31 (d, J=18.4 Hz, 2H) ppm; ¹⁹F NMR (376 MHz, methanol-d4) δ−61.55, −81.09, −84.99 (d, J=198.5 Hz), −102.39 (d, J=199.0 Hz) ppm; UV/vis lambda max 230, 281, 356 nm; ESI-MS m/z calc. 515.12036, found 516.1 (M+1)⁺; Retention time: 1.25 minutes (LC Method Q).

TABLE 5
Final Examples Prepared by Method 14 and Following the Procedure
Described for Representative Example 163

| Example | Amine Structure Amine Method of | Carboxylic Acid | Final Product | Final Product IUPAC |
|---|---|---|---|---|
| number | Preparation (if synthesized) Mass Spec data | Structure | structure | Name |
| 164 | 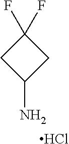 | 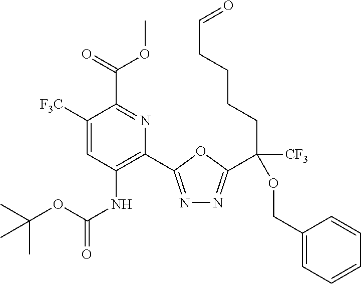 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 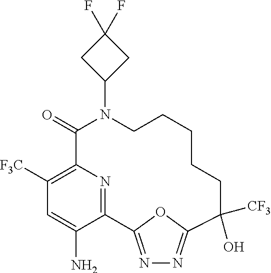 [SMILES: Nc1cc(C(F)(F)F)c2nc1-c1nnc(o1)C(O)(C(F)(F)F)CCCCCN(C1CC(F)(F)C1)C2=O] | 17-amino-12-(3,3- difluorocyclobutyl)- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 200) |
| 165 | 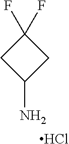 | 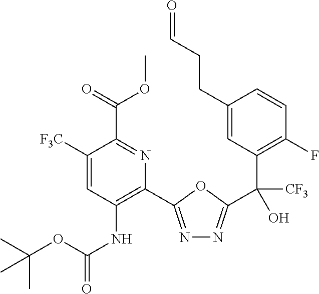 [SMILES: COC(=O)c1nc(-c2nnc(C(O)(c3cc(CCC=O)ccc3F)C(F)(F)F)o2)c(NC(=O)OC(C)(C)C)cc1C(F)(F)F] | 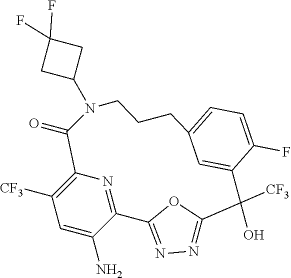 [SMILES: Nc1cc(C(F)(F)F)c2nc1-c1nnc(o1)C(O)(C(F)(F)F)c1cc(ccc1F)CCCN(C1CC(F)(F)C1)C2=O] | 20-amino-15-(3,3- difluorocyclobutyl)- 8-fluoro-6-hydroxy- 6,18- bis(trifluoromethyl)- 23-oxa-3,4,15,21- tetraazatetracyclo [15.3.1.12,5.17,11] tricosa 1(21),2,4,7(22),8,10, 17,19-octaen-16-one (racemic) (Compound 201) |
| 166 | 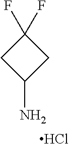 | 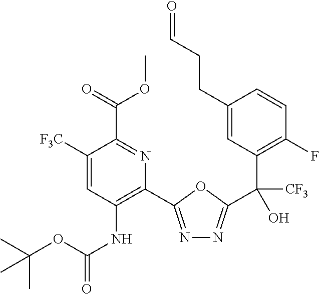 [SMILES: COC(=O)c1nc(-c2nnc(C(O)(c3cc(CCC=O)ccc3F)C(F)(F)F)o2)c(NC(=O)OC(C)(C)C)cc1C(F)(F)F] | 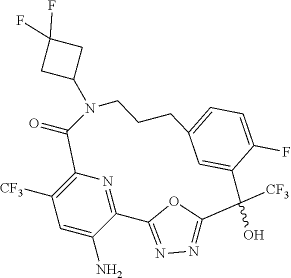 [SMILES: Nc1cc(C(F)(F)F)c2nc1-c1nnc(o1)C(O)(C(F)(F)F)c1cc(ccc1F)CCCN(C1CC(F)(F)C1)C2=O] | 20-amino-15-(3,3- difluorocyclobutyl)- 8-fluoro-6-hydroxy- 6,18- bis(trifluoromethyl)- 23-oxa-3,4,15,21- tetraazatetracyclo [15.3.1.12,5.17,11] tricosa 1(21),2,4,7(22),8,10, 17,19-octaen-16-one (enantiomer 1) (Compound 202) |
| | | | enantiomer 1 | |
| 167 | 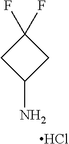 | 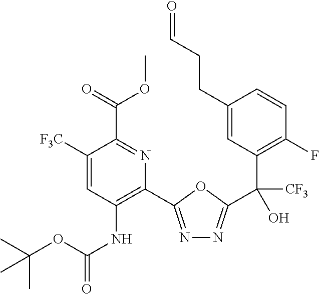 [SMILES: COC(=O)c1nc(-c2nnc(C(O)(c3cc(CCC=O)ccc3F)C(F)(F)F)o2)c(NC(=O)OC(C)(C)C)cc1C(F)(F)F] | 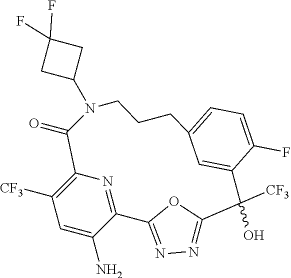 [SMILES: Nc1cc(C(F)(F)F)c2nc1-c1nnc(o1)C(O)(C(F)(F)F)c1cc(ccc1F)CCCN(C1CC(F)(F)C1)C2=O] | 20-amino-15-(3,3- difluorocyclobutyl)- 8-fluoro-6-hydroxy- 6,18- bis(trifluoromethyl)- 23-oxa-3,4,15,21- tetraazatetracyclo [15.3.1.12,5.17,11] tricosa 1(21),2,4,7(22),8,10, 17,19-octaen-16-one (enantiomer 2) (Compound 203) |
| | | | enantiomer 2 | |
| 168 | 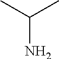 | 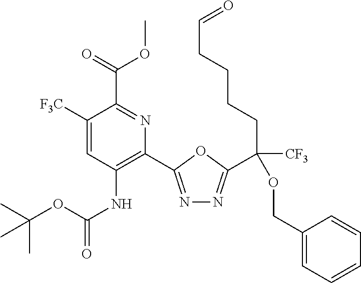 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 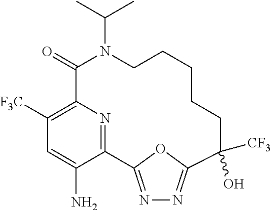 [SMILES: CC(C)N1CCCCCC(O)(C(F)(F)F)c2nnc(o2)-c2nc(c(C(F)(F)F)cc2N)C1=O] | 17-amino-6-hydroxy- 12-isopropyl-6,15- bis(trifluoromethyl)- 19-oxa-3,4,12,18- tetrazatricyclo [12.3.1.12,5] nonadeca- 1(18),2,4,14,16- pentaen-13-one (enantiomer 1) (Compound 204) |
| | | | enantiomer 1 | |
| 169 | 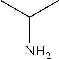 | 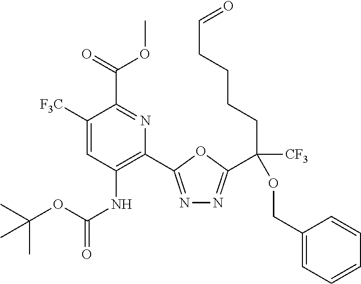 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 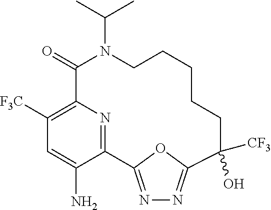 [SMILES: CC(C)N1CCCCCC(O)(C(F)(F)F)c2nnc(o2)-c2nc(c(C(F)(F)F)cc2N)C1=O] | 17-amino-6-hydroxy- 12-isopropyl-6,15- bis(trifluoromethyl)- 19-oxa-3,4,12,18- tetrazatricyclo [12.3.1.12,5] nonadeca- 1(18),2,4,14,16- pentaen-13-one (enantiomer 2) (Compound 205) |
| | | | enantiomer 2 | |
| 170 | 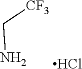 | 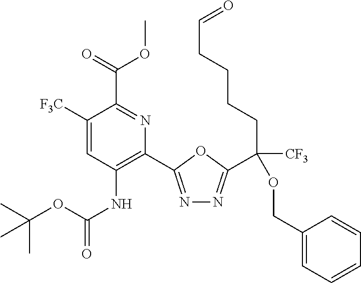 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 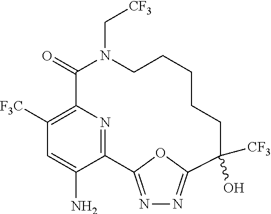 [SMILES: Nc1cc(C(F)(F)F)c2nc1-c1nnc(o1)C(O)(C(F)(F)F)CCCCCN(CC(F)(F)F)C2=O] | 17-amino-6-hydroxy- 12-(2,2,2- trifluoroethyl)-6,15- bis(trifluoromethyl)- 19-oxa-3,4,12,18- tetrazatricyclo [12.3.1.12,5] nonadeca- 1(18),2,4,14,16- pentaen-13-one (enantiomer 1) (Compound 206) |
| | | | enantiomer 1 | |
| 171 | 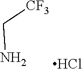 | 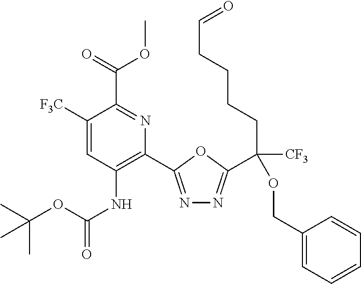 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 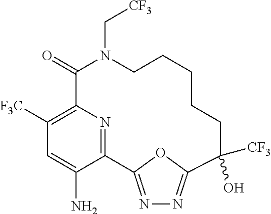 [SMILES: Nc1cc(C(F)(F)F)c2nc1-c1nnc(o1)C(O)(C(F)(F)F)CCCCCN(CC(F)(F)F)C2=O] | 17-amino-6-hydroxy- 12-(2,2,2- trifluoroethyl)-6,15- bis(trifluoromethyl)- 19-oxa-3,4,12,18- tetrazatricyclo [12.3.1.12,5] nonadeca- 1(18),2,4,14,16- pentaen-13-one (enantiomer 2) (Compound 207) |
| | | | enantiomer 2 | |
| 172 | 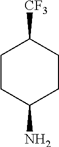 [SMILES: N[C@H]1CC[C@@H](C(F)(F)F)CC1] | 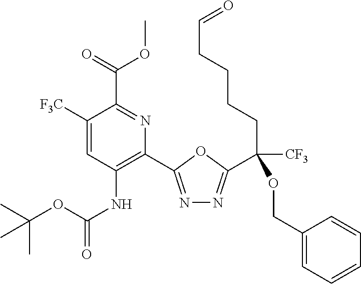 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 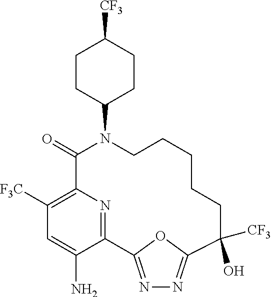 | (6R)-17-amino-6- hydroxy-12-[(1s,4s)- 4- (trifluoromethyl) cyclohexyl]-6,15- bis(trifluoromethyl)- 19-oxa-3,4,12,18- tetraazatricyclo [12.3.1.12,5] nonadeca- 1(18),2,4,14,16- pentaen-13-one (Compound 208) |
| 173 | 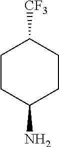 [SMILES: N[C@H]1CC[C@H](C(F)(F)F)CC1] | 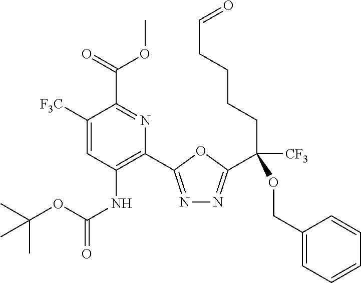 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 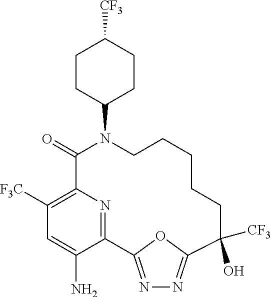 | (6R)-17-amino-6- hydroxy-12-[(1r,4r)- 4- (trifluoromethyl) cyclohexyl]-6,15- bis(trifluoromethyl)- 19-oxa-3,4,12,18- tetraazatricyclo [12.3.1.12,5] nonadeca- 1(18),2,4,14,16- pentaen-13-one (Compound 209) |
| 174 | 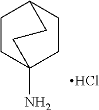 | 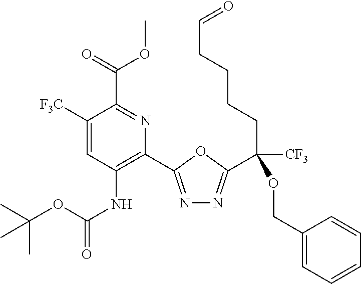 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 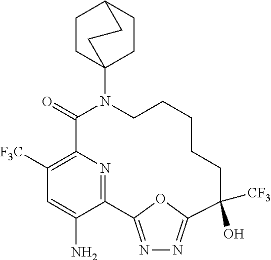 [SMILES: Nc1cc(C(F)(F)F)c2nc1-c1nnc(o1)[C@@](O)(C(F)(F)F)CCCCCN(C13CCC(CC1)CC3)C2=O] | (6R)-17-amino-12-(1- bicyclo[2.2.2]octanyl)- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 210) |
| 175 | 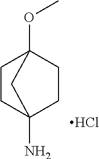 | 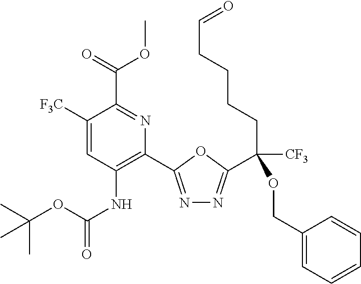 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 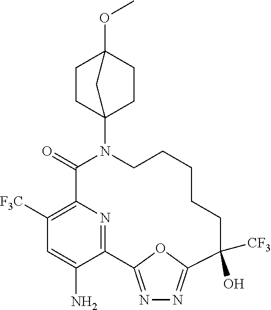 [SMILES: COC12CCC(N3CCCCC[C@](O)(C(F)(F)F)c4nnc(o4)-c4nc(c(C(F)(F)F)cc4N)C3=O)(CC1)C2] | (6R)-17-amino-6- hydroxy-12-(4- methoxynorbornan-1- yl)-6,15- bis(trifluoromethyl)- 19-oxa-3,4,12,18- tetrazatricyclo [12.3.1.12,5] nonadeca- 1(18),2,4,14,16- pentaen-13-one (Compound 211) |
| 176 | 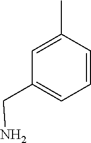 | 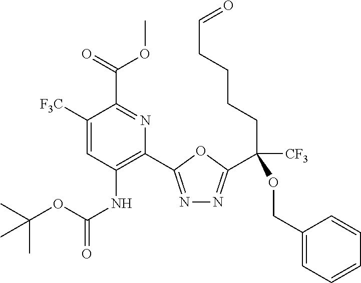 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 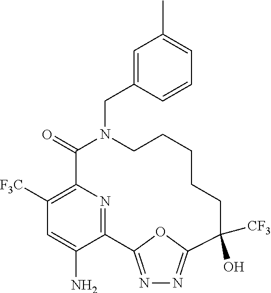 [SMILES: Cc1cccc(CN2CCCCC[C@](O)(C(F)(F)F)c3nnc(o3)-c3nc(c(C(F)(F)F)cc3N)C2=O)c1] | (6R)-17-amino-6- hydroxy-12-(m- tolylmethyl)-6,15- bis(trifluoromethyl)- 19-oxa-3,4,12,18- tetrazatricyclo [12.3.1.12,5] nonadeca- 1(18),2,4,14,16- pentaen-13-one (Compound 212) |
| 177 | 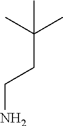 | 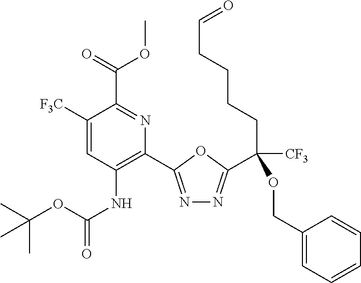 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 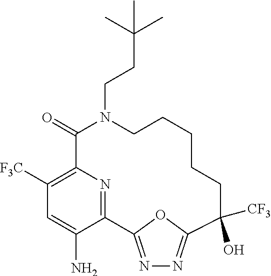 [SMILES: CC(C)(C)CCN1CCCCC[C@](O)(C(F)(F)F)c2nnc(o2)-c2nc(c(C(F)(F)F)cc2N)C1=O] | (6R)-17-amino-12- (3,3-dimethylbutyl)- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 213) |
| 178 | 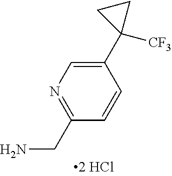 [SMILES: NCc1ccc(C2(C(F)(F)F)CC2)cn1] | 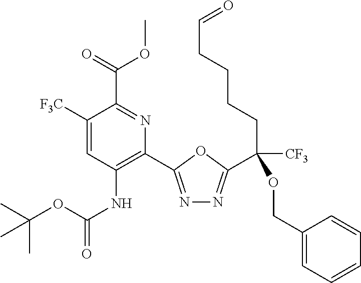 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 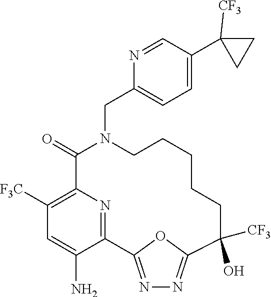 [SMILES: Nc1cc(C(F)(F)F)c2nc1-c1nnc(o1)[C@@](O)(C(F)(F)F)CCCCCN(Cc1ccc(C3(C(F)(F)F)CC3)cn1)C2=O] | (6R)-17-amino-6- hydroxy-6,15- bis(trifluoromethyl)- 12-[[5-[1- (trifluoromethyl) cyclopropyl]-2- pyridyl]methyl]-19- oxa-3,4,12,18- tetrazatricyclo [12.3.1.12,5] nonadeca- 1(18),2,4,14,16- pentaen-13-one (Compound 214) |
| | Method 1 | | | |
| | ESI-MS m/z | | | |
| | calc. | | | |
| | 216.08743, | | | |
| | found 217.1 | | | |
| | (M + 1)⁺ | | | |
| 179 | 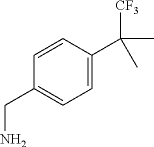 | 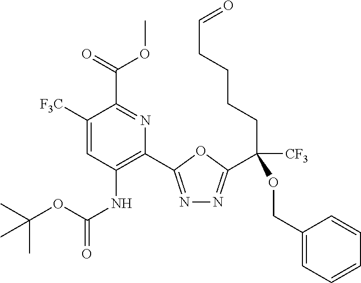 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 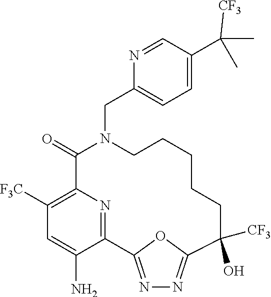 [SMILES: CC(C)(c1ccc(CN2CCCCC[C@](O)(C(F)(F)F)c3nnc(o3)-c3nc(c(C(F)(F)F)cc3N)C2=O)nc1)C(F)(F)F] | (6R)-17-amino-6- hydroxy-12-[[4- (2,2,2-trifluoro-1,1- dimethyl- ethyl)phenyl]methyl]- 6,15- bis(trifluoromethyl)- 19-oxa-3,4,12,18- tetrazatricyclo [12.3.1.12,5] nonadeca- 1(18),2,4,14,16- pentaen-13-one (Compound 215) |
| 180 | 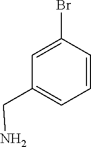 | 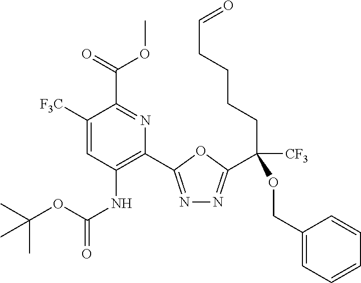 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 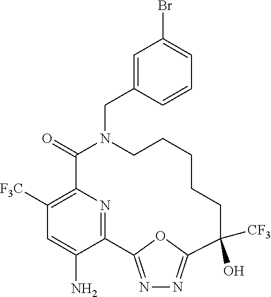 [SMILES: Nc1cc(C(F)(F)F)c2nc1-c1nnc(o1)[C@@](O)(C(F)(F)F)CCCCCN(Cc1cccc(Br)c1)C2=O] | (6R)-17-amino-12- [(3- bromophenyl)methyl]- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 216) |
| 181 | 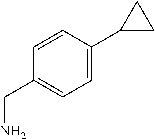 [SMILES: NCc1ccc(C2CC2)cc1] | 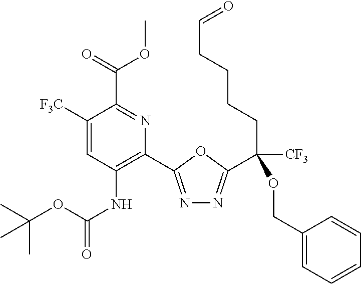 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 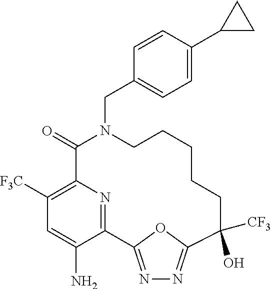 [SMILES: Nc1cc(C(F)(F)F)c2nc1-c1nnc(o1)[C@@](O)(C(F)(F)F)CCCCCN(Cc1ccc(C3CC3)cc1)C2=O] | (6R)-17-amino-12- [(4- cyclopropylphenyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 217) |
| 182 | 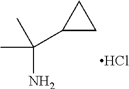 | 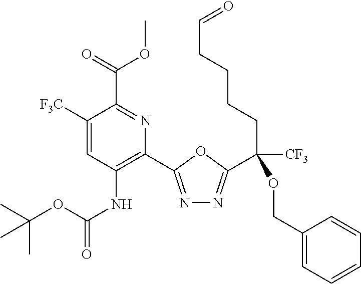 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 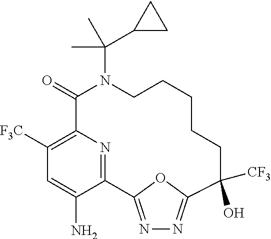 [SMILES: CC(C)(C1CC1)N1CCCCC[C@](O)(C(F)(F)F)c2nnc(o2)-c2nc(c(C(F)(F)F)cc2N)C1=O] | (6R)-17-amino-12-(1- cyclopropyl-1- methyl-ethyl)-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 218) |
| 183 | 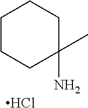 | 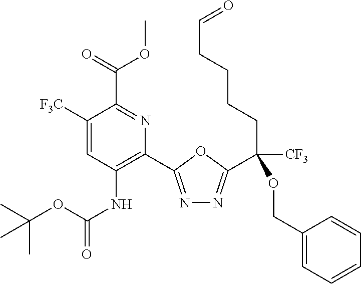 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 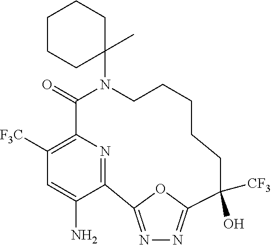 [SMILES: CC1(N2CCCCC[C@](O)(C(F)(F)F)c3nnc(o3)-c3nc(c(C(F)(F)F)cc3N)C2=O)CCCCC1] | (6R)-17-amino-6- hydroxy-12-(1- methylcyclohexyl)- 6,15- bis(trifluoromethyl)- 19-oxa-3,4,12,18- tetrazatricyclo [12.3.1.12,5] nonadeca- 1(18),2,4,14,16- pentaen-13-one (Compound 219) |
| 184 | 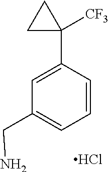 [SMILES: NCc1cccc(C2(C(F)(F)F)CC2)c1 ; Cl] | 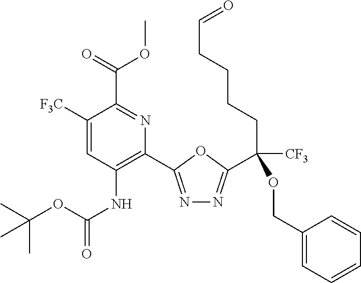 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 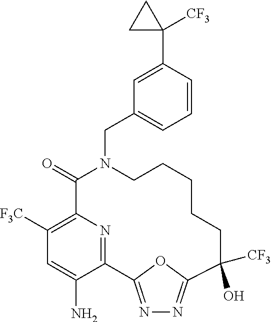 [SMILES: Nc1cc(C(F)(F)F)c2nc1-c1nnc(o1)[C@@](O)(C(F)(F)F)CCCCCN(Cc1cccc(C3(C(F)(F)F)CC3)c1)C2=O] | (6R)-17-amino-6- hydroxy-6,15- bis(trifluoromethyl)- 12-[[3-[1- (trifluoromethyl) cyclopropyl]phenyl] methyl]-19-oxa- 3,4,12,18- tetrazatricyclo [12.3.1.12,5] nonadeca- 1(18),2,4,14,16- pentaen-13-one (Compound 220) |
| | Method 1* | | | |
| | ESI-MS m/z | | | |
| | calc. | | | |
| | 215.09218, | | | |
| | found 216.1 | | | |
| | (M + 1)⁺ | | | |
| 185 | 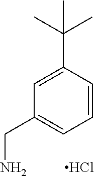 | 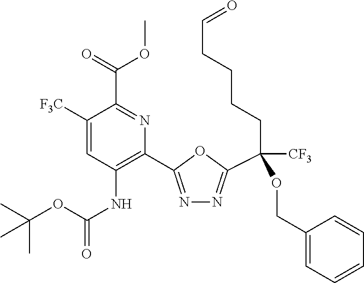 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 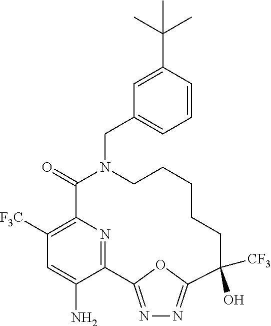 [SMILES: CC(C)(C)c1cccc(CN2CCCCC[C@](O)(C(F)(F)F)c3nnc(o3)-c3nc(c(C(F)(F)F)cc3N)C2=O)c1] | (6R)-17-amino-12- [(3-tert- butylphenyl)methyl]- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 221) |
| 186 | 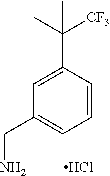 | 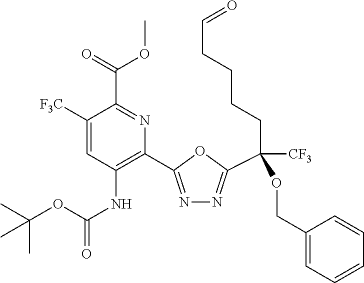 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 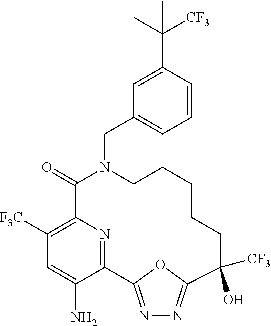 [SMILES: CC(C)(c1cccc(CN2CCCCC[C@](O)(C(F)(F)F)c3nnc(o3)-c3nc(c(C(F)(F)F)cc3N)C2=O)c1)C(F)(F)F] | (6R)-17-amino-6- hydroxy-12-[[3- (2,2,2-trifluoro-1,1- dimethyl- ethyl)phenyl]methyl]- 6,15- bis(trifluoromethyl)- 19-oxa-3,4,12,18- tetrazatricyclo [12.3.1.12,5] nonadeca- 1(18),2,4,14,16- pentaen-13-one (Compound 222) |
| | Method 1* | | | |
| | ESI-MS m/z | | | |
| | calc. | | | |
| | 217.10783, | | | |
| | found 218.1 | | | |
| | (M + 1)⁺ | | | |
| 187 | 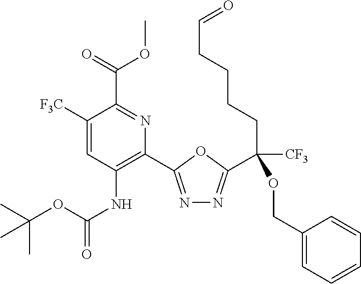 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 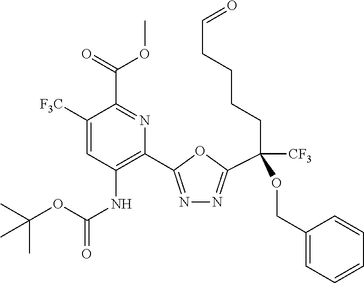 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 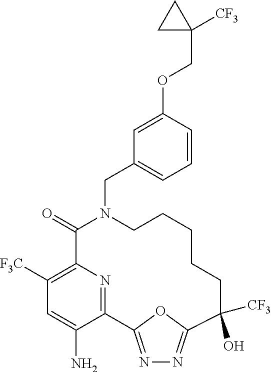 [SMILES: Nc1cc(C(F)(F)F)c2nc1-c1nnc(o1)[C@@](O)(C(F)(F)F)CCCCCN(Cc1cccc(OCC3(C(F)(F)F)CC3)c1)C2=O] | (6R)-17-amino-6- hydroxy-6,15- bis(trifluoromethyl)- 12-[[3-[[1- (trifluoromethyl) cyclopropyl]methoxy] phenyl]methyl]- 19-oxa-3,4,12,18- tetrazatricyclo [12.3.1.12,5] nonadeca- 1(18),2,4,14,16- pentaen-13-one (Compound 223) |
| | Method 2 | | | |
| | ESI-MS m/z | | | |
| | calc. | | | |
| | 245.10275, | | | |
| | found 246.2 | | | |
| | (M + 1)⁺ | | | |
| 188 | 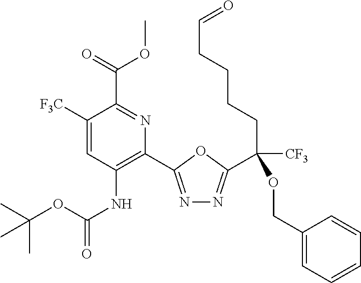 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 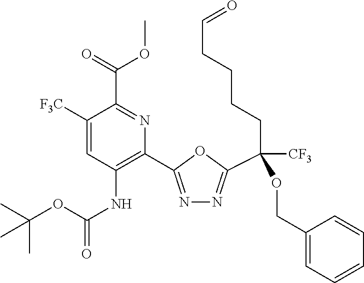 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 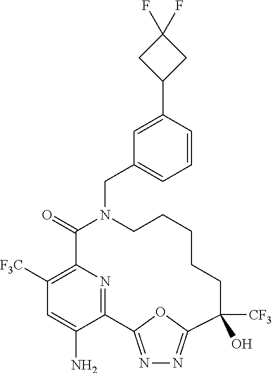 [SMILES: Nc1cc(C(F)(F)F)c2nc1-c1nnc(o1)[C@@](O)(C(F)(F)F)CCCCCN(Cc1cccc(C3CC(F)(F)C3)c1)C2=O] | (6R)-17-amino-12- [[3-(3,3- difluorocyclobutyl) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 224) |
| | Method 3 | | | |
| | ESI-MS m/z | | | |
| | calc. | | | |
| | 197.10161, | | | |
| | found 198.1 | | | |
| | (M + 1)⁺ | | | |
| 189 | 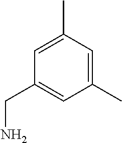 | 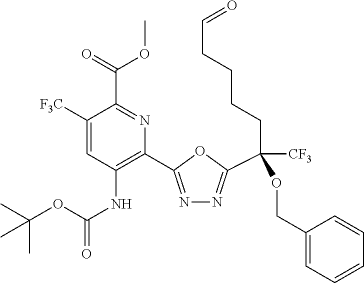 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 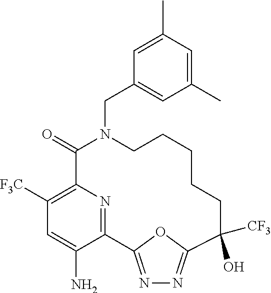 [SMILES: Cc1cc(C)cc(CN2CCCCC[C@](O)(C(F)(F)F)c3nnc(o3)-c3nc(c(C(F)(F)F)cc3N)C2=O)c1] | (6R)-17-amino-12- [(3,5- dimethylphenyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 225) |
| 190 | 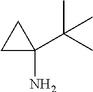 | 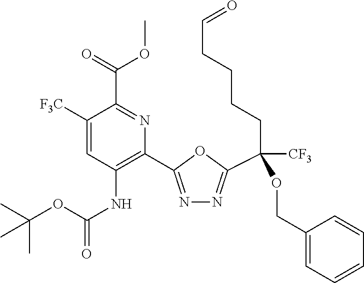 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 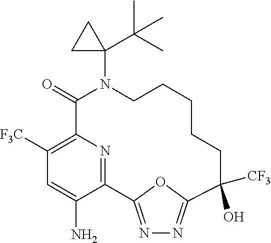 [SMILES: CC(C)(C)C1(N2CCCCC[C@](O)(C(F)(F)F)c3nnc(o3)-c3nc(c(C(F)(F)F)cc3N)C2=O)CC1] | (6R)-17-amino-12-(1- tert- butylcyclopropyl)-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 226) |
| 191 | 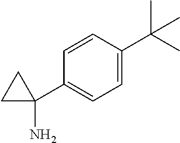 | 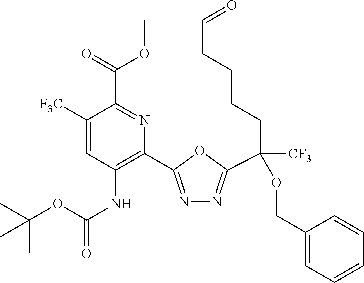 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 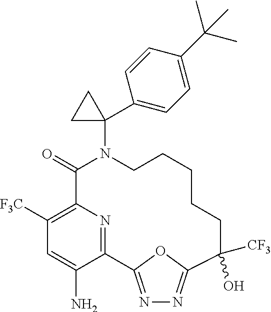 [SMILES: CC(C)(C)c1ccc(C2(N3CCCCCC(O)(C(F)(F)F)c4nnc(o4)-c4nc(c(C(F)(F)F)cc4N)C3=O)CC2)cc1] | 17-amino-12-[1-(4- tert- butylphenyl)cyclo- propyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (enantiomer 1) (Compound 227) |
| | | | enantiomer 1 | |
| 192 | 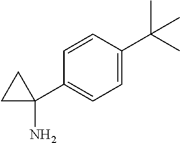 | 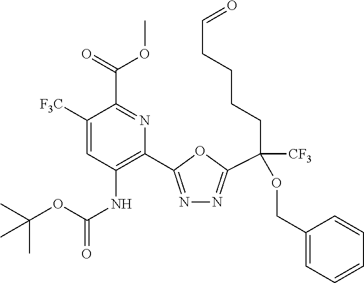 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 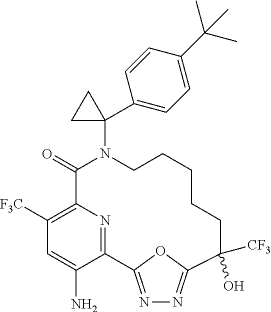 [SMILES: CC(C)(C)c1ccc(C2(N3CCCCCC(O)(C(F)(F)F)c4nnc(o4)-c4nc(c(C(F)(F)F)cc4N)C3=O)CC2)cc1] | 17-amino-12-[1-(4- tert- butylphenyl)cyclo- propyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (enantiomer 2) (Compound 228) |
| | | | enantiomer 2 | |
| 193 | 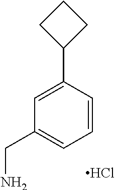 [SMILES: NCc1cccc(C2CCC2)c1 ; Cl] | 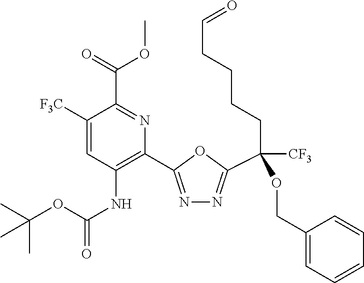 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 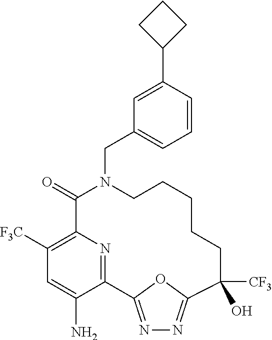 [SMILES: Nc1cc(C(F)(F)F)c2nc1-c1nnc(o1)[C@@](O)(C(F)(F)F)CCCCCN(Cc1cccc(C3CCC3)c1)C2=O] | (6R)-17-amino-12- [(3- cyclobutylphenyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 229) |
| | Method 4 | | | |
| | ESI-MS m/z | | | |
| | calc. | | | |
| | 161.12045, | | | |
| | found 162.2 | | | |
| | (M + 1)⁺ | | | |
| 194 | 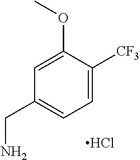 | 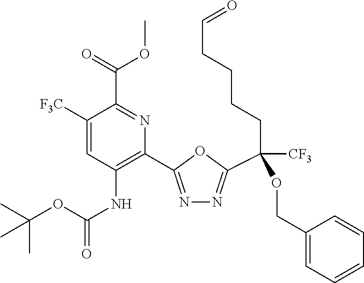 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 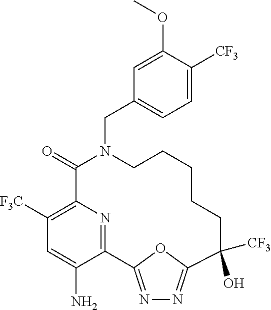 [SMILES: COc1cc(CN2CCCCC[C@](O)(C(F)(F)F)c3nnc(o3)-c3nc(c(C(F)(F)F)cc3N)C2=O)ccc1C(F)(F)F] | (6R)-17-amino-6- hydroxy-12-[[3- methoxy-4- (trifluoromethyl) phenyl]methyl]-6,15- bis(trifluoromethyl)- 19-oxa-3,4,12,18- tetrazatricyclo [12.3.1.12,5] nonadeca- 1(18),2,4,14,16- pentaen-13-one (Compound 230) |
| | Method 5 | | | |
| | ESI-MS m/z | | | |
| | calc. | | | |
| | 205.07144, | | | |
| | found 206.1 | | | |
| | (M + 1)⁺ | | | |
| 195 | 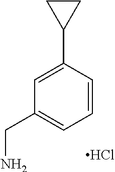 [SMILES: NCc1cccc(C2CC2)c1 ; Cl] | 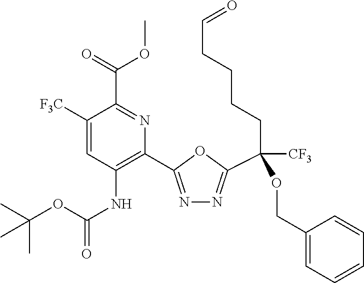 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 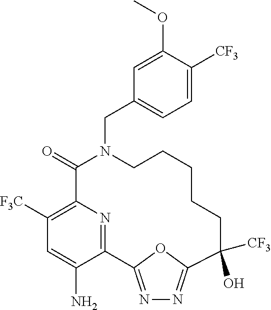 [SMILES: COc1cc(CN2CCCCC[C@](O)(C(F)(F)F)c3nnc(o3)-c3nc(c(C(F)(F)F)cc3N)C2=O)ccc1C(F)(F)F] | (6R)-17-amino-12- [(3- cyclopropylphenyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 231) |
| | Method 6 | | | |
| | ESI-MS m/z | | | |
| | calc. | | | |
| | 147.1048, | | | |
| | found | | | |
| | 149.17 | | | |
| | (M + 1)⁺ | | | |
| 196 | 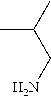 | 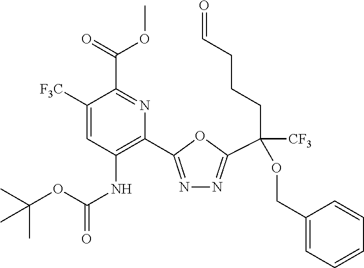 [SMILES: COC(=O)c1nc(-c2nnc(C(CCCC=O)(OCc3ccccc3)C(F)(F)F)o2)c(NC(=O)OC(C)(C)C)cc1C(F)(F)F] | 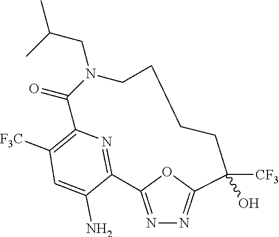 [SMILES: CC(C)CN1CCCCC(O)(C(F)(F)F)c2nnc(o2)-c2nc(c(C(F)(F)F)cc2N)C1=O] | 16-amino-6-hydroxy- 11-isobutyl-6,14- bis(trifluoromethyl)- 18-oxa-3,4,11,17- tetrazatricyclo [11.3.1.12,5] octadeca- 1(17),2,4,13,15- pentaen-12-one (enantiomer 1) (Compound 232) |
| | | | enantiomer 1 | |
| 197 | 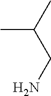 | 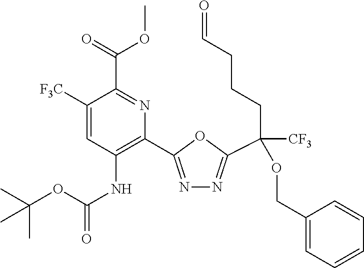 [SMILES: COC(=O)c1nc(-c2nnc(C(CCCC=O)(OCc3ccccc3)C(F)(F)F)o2)c(NC(=O)OC(C)(C)C)cc1C(F)(F)F] | 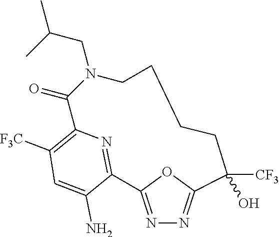 [SMILES: CC(C)CN1CCCCC(O)(C(F)(F)F)c2nnc(o2)-c2nc(c(C(F)(F)F)cc2N)C1=O] | 16-amino-6-hydroxy- 11-isobutyl-6,14- bis(trifluoromethyl)- 18-oxa-3,4,11,17- tetrazatricyclo [11.3.1.12,5] octadeca- 1(17),2,4,13,15- pentaen-12-one (enantiomer 2) (Compound 233) |
| | | | enantiomer 2 | |
| 198 | 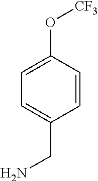 [SMILES: NCc1ccc(OC(F)(F)F)cc1] | 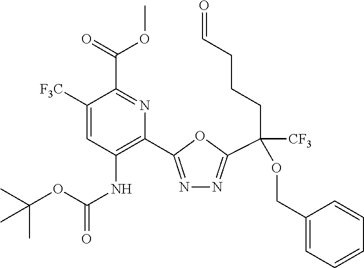 [SMILES: COC(=O)c1nc(-c2nnc(C(CCCC=O)(OCc3ccccc3)C(F)(F)F)o2)c(NC(=O)OC(C)(C)C)cc1C(F)(F)F] | 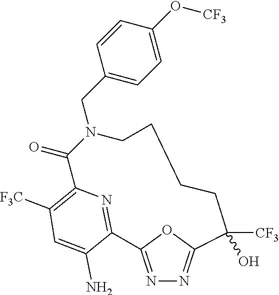 [SMILES: Nc1cc(C(F)(F)F)c2nc1-c1nnc(o1)C(O)(C(F)(F)F)CCCCN(Cc1ccc(OC(F)(F)F)cc1)C2=O] | 16-amino-6-hydroxy- 11-[[4- (trifluoromethoxy) phenyl]methyl]-6,14- bis(trifluoromethyl)- 18-oxa-3,4,11,17- tetrazatricyclo [11.3.1.12,5] octadeca- 1(17),2,4,13,15- pentaen-12-one (enantiomer 1) (Compound 234) |
| | | | enantiomer 1 | |
| 199 | 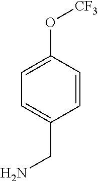 [SMILES: NCc1ccc(OC(F)(F)F)cc1] | 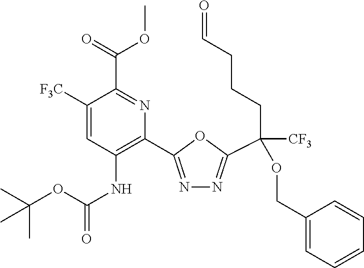 [SMILES: COC(=O)c1nc(-c2nnc(C(CCCC=O)(OCc3ccccc3)C(F)(F)F)o2)c(NC(=O)OC(C)(C)C)cc1C(F)(F)F] | 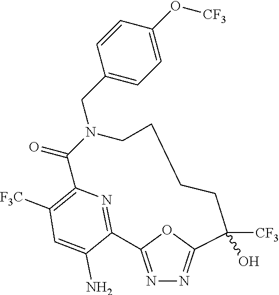 [SMILES: Nc1cc(C(F)(F)F)c2nc1-c1nnc(o1)C(O)(C(F)(F)F)CCCCN(Cc1ccc(OC(F)(F)F)cc1)C2=O] | 16-amino-6-hydroxy- 11-[[4- (trifluoromethoxy) phenyl]methyl]-6,14- bis(trifluoromethyl)- 18-oxa-3,4,11,17- tetrazatricyclo [11.3.1.12,5] octadeca- 1(17),2,4,13,15- pentaen-12-one (enantiomer 1) (Compound 235) |
| | | | enantiomer 2 | |
| 200 | 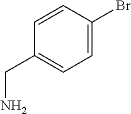 | 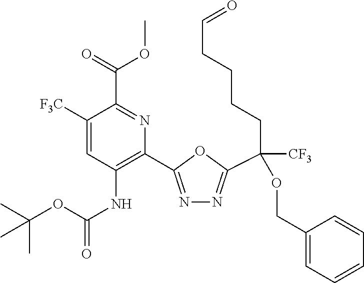 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 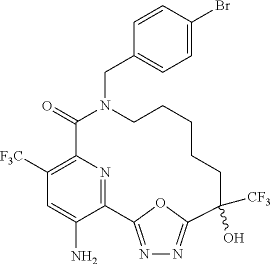 [SMILES: Nc1cc(C(F)(F)F)c2nc1-c1nnc(o1)C(O)(C(F)(F)F)CCCCCN(Cc1ccc(Br)cc1)C2=O] | 17-amino-12-[(4- bromophenyl)methyl]- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (enantiomer 1) (Compound 236) |
| | | | enantiomer 1 | |
| 201 | 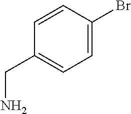 | 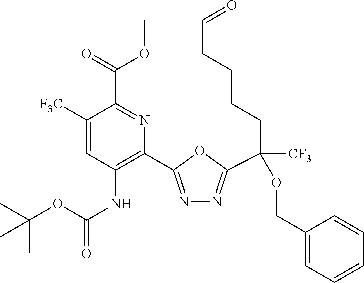 [SMILES: COC(=O)c1nc(-c2nnc(C(CCCCC=O)(OCc3ccccc3)C(F)(F)F)o2)c(NC(=O)OC(C)(C)C)cc1C(F)(F)F] | 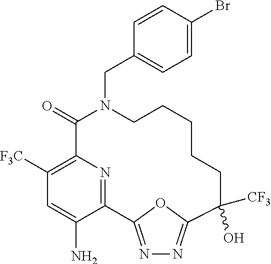 [SMILES: Nc1cc(C(F)(F)F)c2nc1-c1nnc(o1)C(O)(C(F)(F)F)CCCCCN(Cc1ccc(Br)cc1)C2=O] | 17-amino-12-[(4- bromophenyl)methyl]- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (enantiomer 2) (Compound 237) |
| | | | enantiomer 2 | |
| 202 | 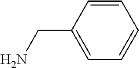 | 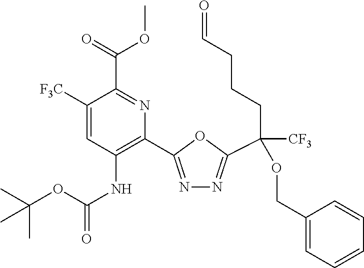 [SMILES: COC(=O)c1nc(-c2nnc(C(CCCC=O)(OCc3ccccc3)C(F)(F)F)o2)c(NC(=O)OC(C)(C)C)cc1C(F)(F)F] | 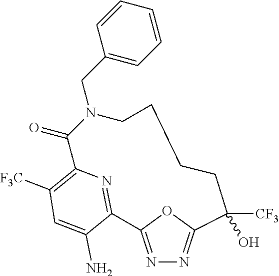 [SMILES: Nc1cc(C(F)(F)F)c2nc1-c1nnc(o1)C(O)(C(F)(F)F)CCCCN(Cc1ccccc1)C2=O] | 16-amino-11-benzyl- 6-hydroxy-6,14- bis(trifluoromethyl)- 18-oxa-3,4,11,17- tetrazatricyclo [11.3.1.12,5] octadeca- 1(17),2,4,13,15- pentaen-12-one (enantiomer 1) (Compound 238) |
| | | | enantiomer 1 | |
| 203 | 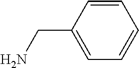 | 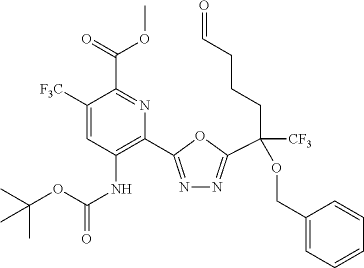 [SMILES: COC(=O)c1nc(-c2nnc(C(CCCC=O)(OCc3ccccc3)C(F)(F)F)o2)c(NC(=O)OC(C)(C)C)cc1C(F)(F)F] | 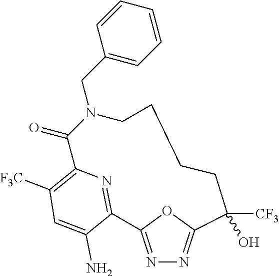 [SMILES: Nc1cc(C(F)(F)F)c2nc1-c1nnc(o1)C(O)(C(F)(F)F)CCCCN(Cc1ccccc1)C2=O] | 16-amino-11-benzyl- 6-hydroxy-6,14- bis(trifluoromethyl)- 18-oxa-3,4,11,17- tetrazatricyclo [11.3.1.12,5] octadeca- 1(17),2,4,13,15- pentaen-12-one (enantiomer 2) (Compound 239) |
| | | | enantiomer 2 | |
| 204 | 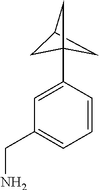 [SMILES: NCc1cccc(C23CC(C2)C3)c1] | 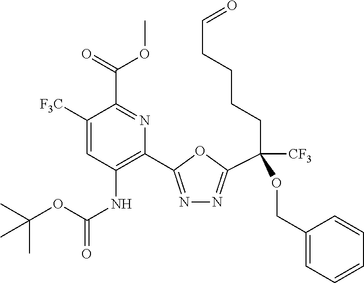 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 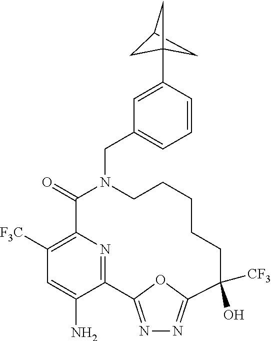 [SMILES: Nc1cc(C(F)(F)F)c2nc1-c1nnc(o1)[C@@](O)(C(F)(F)F)CCCCCN(Cc1cccc(C34CC(C3)C4)c1)C2=O] | (6R)-17-amino-12- [3-(1- bicyclo[1.1.1]pentanyl) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 240) |
| 205 | 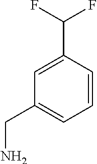 [SMILES: NCc1cccc(C(F)F)c1] | 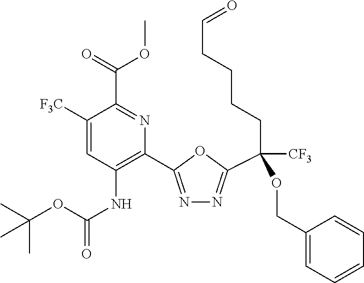 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 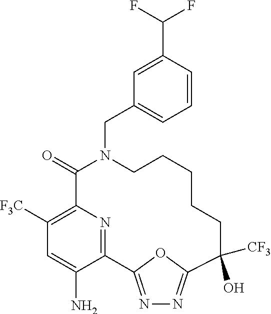 [SMILES: Nc1cc(C(F)(F)F)c2nc1-c1nnc(o1)[C@@](O)(C(F)(F)F)CCCCCN(Cc1cccc(C(F)F)c1)C2=O] | (6R)-17-amino-12- [[3- (difluoromethyl) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 241) |
| 206 | 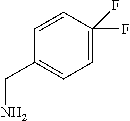 | 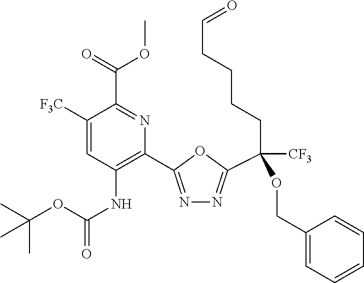 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 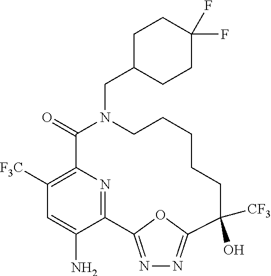 [SMILES: Nc1cc(C(F)(F)F)c2nc1-c1nnc(o1)[C@@](O)(C(F)(F)F)CCCCCN(CC1CCC(F)(F)CC1)C2=O] | (6R)-17-amino-12- [(4,4- difluorocyclohexyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 242) |
| 207 | 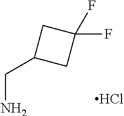 | 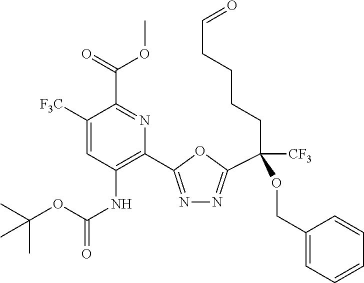 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 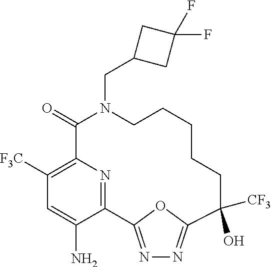 [SMILES: Nc1cc(C(F)(F)F)c2nc1-c1nnc(o1)[C@@](O)(C(F)(F)F)CCCCCN(CC1CC(F)(F)C1)C2=O] | (6R)-17-amino-12- [(3,3- difluorocyclobutyl) methyl]-6-hydroxy- 6,15- bis(trifluoromethyl)- 19-oxa-3,4,12,18- tetrazatricyclo [12.3.1.12,5] nonadeca- 1(18),2,4,14,16- pentaen-13-one (Compound 243) |
| 208 | 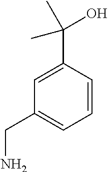 | 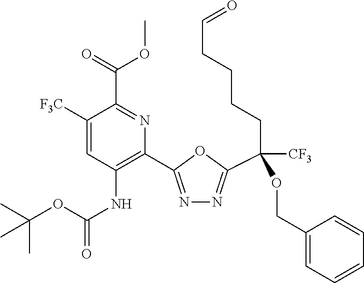 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 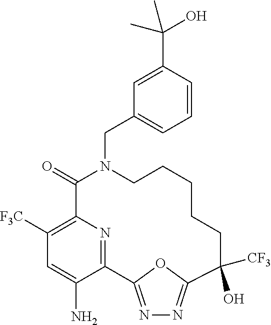 [SMILES: CC(C)(O)c1cccc(CN2CCCCC[C@](O)(C(F)(F)F)c3nnc(o3)-c3nc(c(C(F)(F)F)cc3N)C2=O)c1] | (6R)-17-amino-6- hydroxy-12-[[3-(1- hydroxy-1-methyl- ethyl)phenyl]methyl]- 6,15- bis(trifluoromethyl)- 19-oxa-3,4,12,18- tetrazatricyclo [12.3.1.12,5] nonadeca- 1(18),2,4,14,16- pentaen-13-one (Compound 244) |
| 209 | 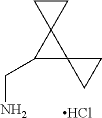 | 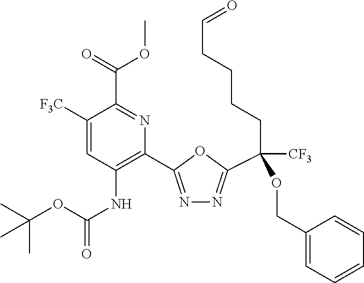 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 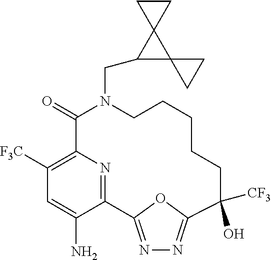 [SMILES: Nc1cc(C(F)(F)F)c2nc1-c1nnc(o1)[C@@](O)(C(F)(F)F)CCCCCN(CC1C3(CC3)C13CC3)C2=O] | (6R)-17-amino-12- (dispiro[2.0.24.13] heptan-7-ylmethyl)-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 245) |
| 210 | 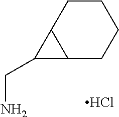 | 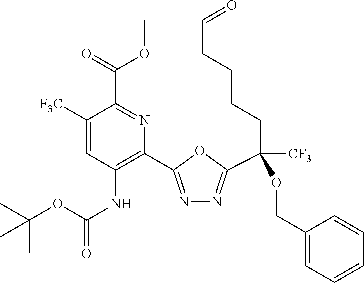 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 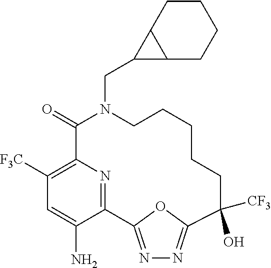 [SMILES: Nc1cc(C(F)(F)F)c2nc1-c1nnc(o1)[C@@](O)(C(F)(F)F)CCCCCN(CC1C3CCCCC31)C2=O] | (6R)-17-amino-6- hydroxy-12- (norcaran-7- ylmethyl)-6,15- bis(trifluoromethyl)- 19-oxa-3,4,12,18- tetrazatricyclo [12.3.1.12,5] nonadeca- 1(18),2,4,14,16- pentaen-13-one (Compound 246) |
| 211 | 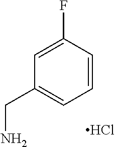 | 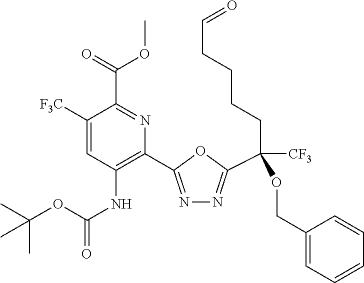 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 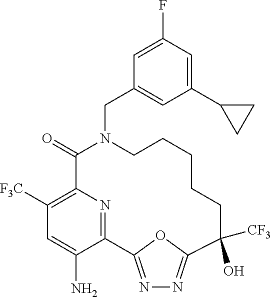 [SMILES: Nc1cc(C(F)(F)F)c2nc1-c1nnc(o1)[C@@](O)(C(F)(F)F)CCCCCN(Cc1cc(F)cc(C3CC3)c1)C2=O] | (6R)-17-amino-12- [(3-cyclopropyl-5- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 247) |
| | Method 6 | | | |
| | ESI-MS m/z | | | |
| | calc. | | | |
| | 165.09538, | | | |
| | found 166.2 | | | |
| | (M + 1)⁺ | | | |
| 212 | 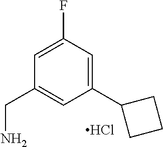 [SMILES: NCc1cc(F)cc(C2CCC2)c1 ; Cl] | 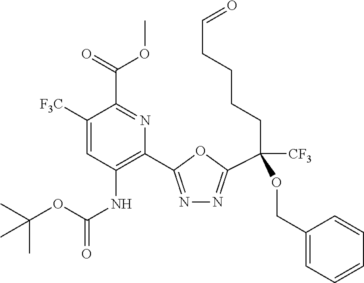 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 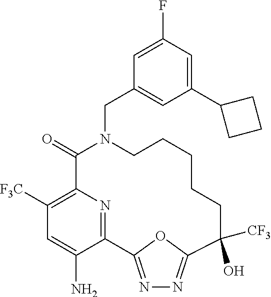 [SMILES: Nc1cc(C(F)(F)F)c2nc1-c1nnc(o1)[C@@](O)(C(F)(F)F)CCCCCN(Cc1cc(F)cc(C3CCC3)c1)C2=O] | (6R)-17-amino-12- [(3-cyclobutyl-5- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 248) |
| | Method 4 | | | |
| | ESI-MS m/z | | | |
| | calc. | | | |
| | 179.11102, | | | |
| | found 180.2 | | | |
| | (M + 1)⁺ | | | |
| 213 | 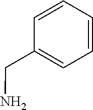 | 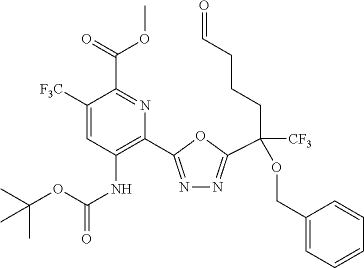 [SMILES: COC(=O)c1nc(-c2nnc(C(CCCC=O)(OCc3ccccc3)C(F)(F)F)o2)c(NC(=O)OC(C)(C)C)cc1C(F)(F)F] | 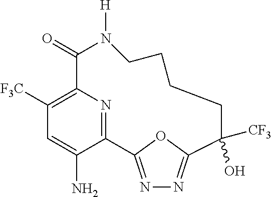 [SMILES: Nc1cc(C(F)(F)F)c2nc1-c1nnc(o1)C(O)(C(F)(F)F)CCCCNC2=O] | 16-amino-6-hydroxy- 6,14- bis(trifluoromethyl)- 18-oxa-3,4,11,17- tetrazatricyclo [11.3.1.12,5] octadeca- 1(17),2,4,13,15- pentaen-12-one (enantiomer 1) (Compound 249) |
| | | | enantiomer 1 | |
| 214 | 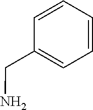 | 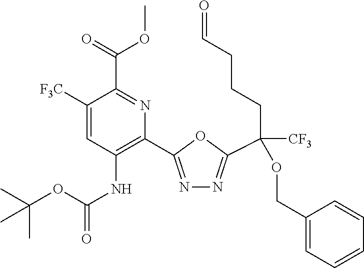 [SMILES: COC(=O)c1nc(-c2nnc(C(CCCC=O)(OCc3ccccc3)C(F)(F)F)o2)c(NC(=O)OC(C)(C)C)cc1C(F)(F)F] | 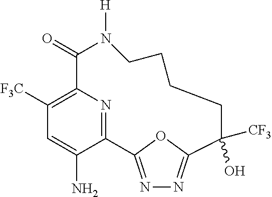 [SMILES: Nc1cc(C(F)(F)F)c2nc1-c1nnc(o1)C(O)(C(F)(F)F)CCCCNC2=O] | 16-amino-6-hydroxy- 6,14- bis(trifluoromethyl)- 18-oxa-3,4,11,17- tetrazatricyclo [11.3.1.12,5] octadeca- 1(17),2,4,13,15- pentaen-12-one (enantiomer 2) (Compound 250) |
| | | | enantiomer 2 | |
| 215 | 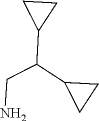 | 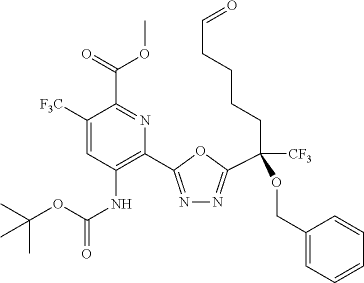 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 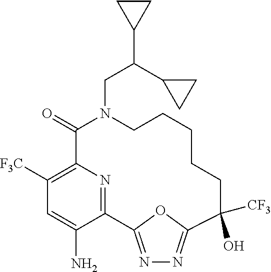 [SMILES: Nc1cc(C(F)(F)F)c2nc1-c1nnc(o1)[C@@](O)(C(F)(F)F)CCCCCN(CC(C1CC1)C1CC1)C2=O] | (6R)-17-amino-12- (2,2- dicyclopropylethyl)- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 251) |
| 216 | 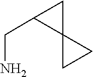 | 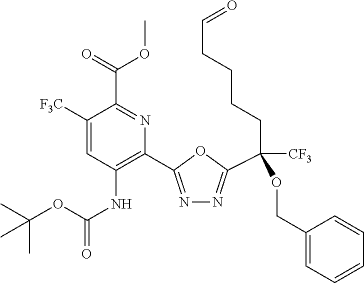 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 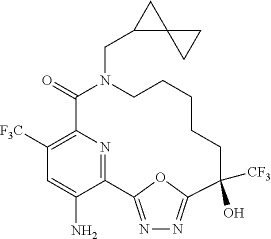 [SMILES: Nc1cc(C(F)(F)F)c2nc1-c1nnc(o1)[C@@](O)(C(F)(F)F)CCCCCN(CC1CC13CC3)C2=O] | (6R)-17-amino-6- hydroxy-12- (spiro[2.2]pentan-2- ylmethyl)-6,15- bis(trifluoromethyl)- 19-oxa-3,4,12,18- tetrazatricyclo [12.3.1.12,5] nonadeca- 1(18),2,4,14,16- pentaen-13-one (Compound 252) |
| 217 | 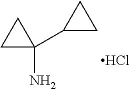 | 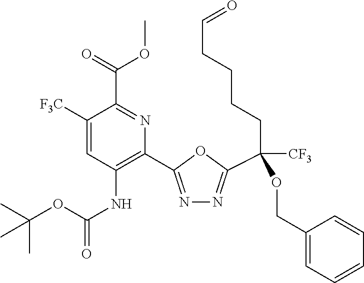 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 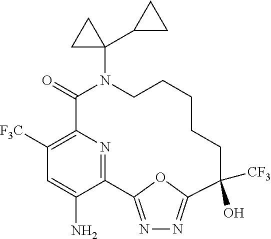 [SMILES: Nc1cc(C(F)(F)F)c2nc1-c1nnc(o1)[C@@](O)(C(F)(F)F)CCCCCN(C1(C3CC3)CC1)C2=O] | (6R)-17-amino-12-(1- cyclopropylcyclo- propyl)- 6-hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 253) |
| 218 | 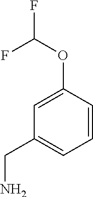 [SMILES: NCc1cccc(OC(F)F)c1] | 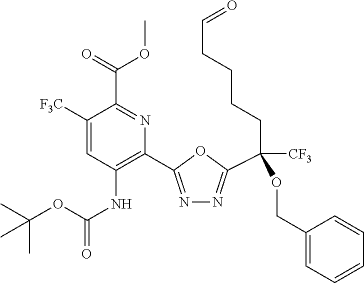 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 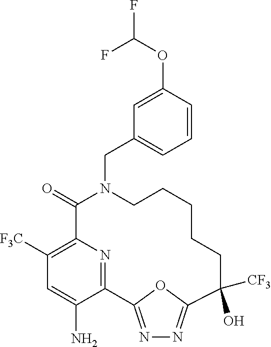 [SMILES: Nc1cc(C(F)(F)F)c2nc1-c1nnc(o1)[C@@](O)(C(F)(F)F)CCCCCN(Cc1cccc(OC(F)F)c1)C2=O] | (6R)-17-amino-12- [[3- (difluoromethoxy) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 254) |
| 219 | 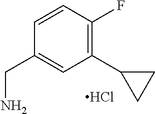 [SMILES: NCc1ccc(F)c(C2CC2)c1 ; Cl] | 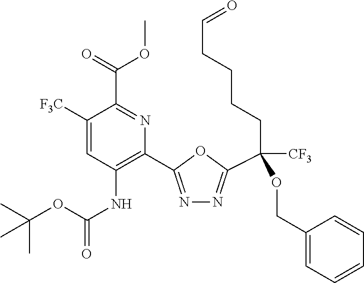 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 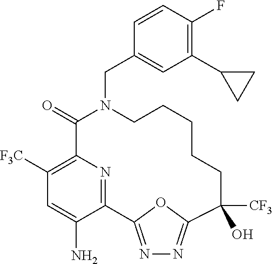 [SMILES: Nc1cc(C(F)(F)F)c2nc1-c1nnc(o1)[C@@](O)(C(F)(F)F)CCCCCN(Cc1ccc(F)c(C3CC3)c1)C2=O] | (6R)-17-amino-12- [(3-cyclopropyl-4- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 255) |
| | Method 6 | | | |
| | ESI-MS m/z | | | |
| | calc. | | | |
| | 165.09538, | | | |
| | found 166.1 | | | |
| | (M + 1)⁺ | | | |
| 220 | 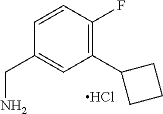 [SMILES: NCc1ccc(F)c(C2CCC2)c1 ; Cl] | 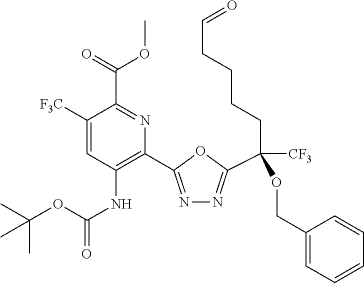 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 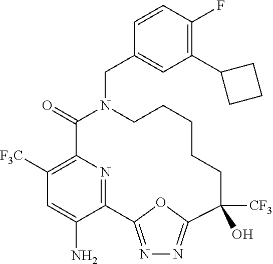 [SMILES: Nc1cc(C(F)(F)F)c2nc1-c1nnc(o1)[C@@](O)(C(F)(F)F)CCCCCN(Cc1ccc(F)c(C3CCC3)c1)C2=O] | (6R)-17-amino-12- [(3-cyclobutyl-4- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 256) |
| | Method 4 | | | |
| | ESI-MS m/z | | | |
| | calc. | | | |
| | 179.11102, | | | |
| | found 180.1 | | | |
| | (M + 1)⁺ | | | |
| 221 | 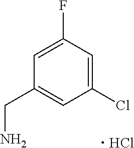 | 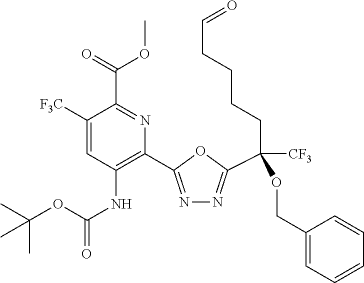 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 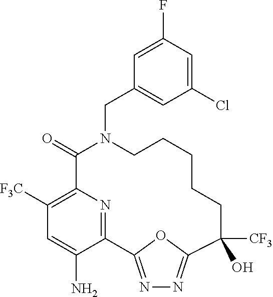 [SMILES: Nc1cc(C(F)(F)F)c2nc1-c1nnc(o1)[C@@](O)(C(F)(F)F)CCCCCN(Cc1cc(F)cc(Cl)c1)C2=O] | (6R)-17-amino-12- [(3-chloro-5-fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 257) |
| 222 | 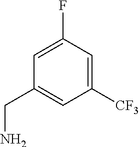 [SMILES: NCc1cc(F)cc(C(F)(F)F)c1] | 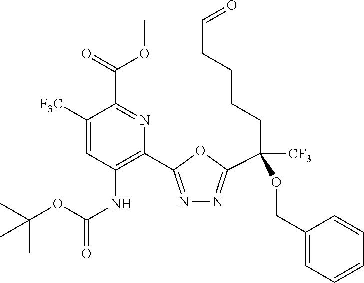 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 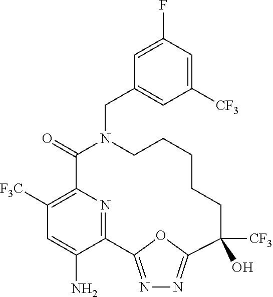 [SMILES: Nc1cc(C(F)(F)F)c2nc1-c1nnc(o1)[C@@](O)(C(F)(F)F)CCCCCN(Cc1cc(F)cc(C(F)(F)F)c1)C2=O] | (6R)-17-amino-12- [[3-fluoro-5- (trifluoromethyl) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 258) |
| 223 | 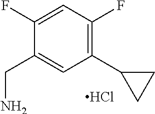 [SMILES: NCc1cc(C2CC2)c(F)cc1F ; Cl] | 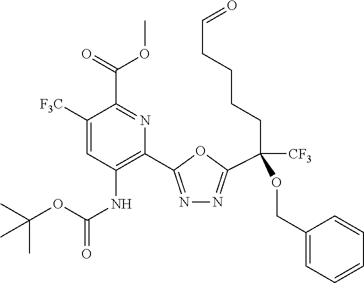 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 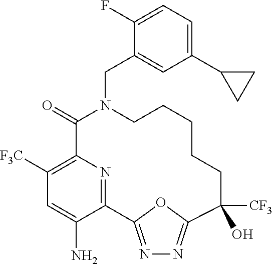 [SMILES: Nc1cc(C(F)(F)F)c2nc1-c1nnc(o1)[C@@](O)(C(F)(F)F)CCCCCN(Cc1cc(C3CC3)ccc1F)C2=O] | (6R)-17-amino-12- [(5-cyclopropyl-2- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 259) |
| | Method 6 | | | |
| | ESI-MS m/z | | | |
| | calc. | | | |
| | 165.09538, | | | |
| | found 166.2 | | | |
| | (M + 1)⁺ | | | |
| 224 | 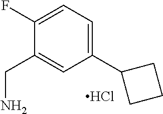 [SMILES: NCc1cc(C2CCC2)ccc1F ; Cl] | 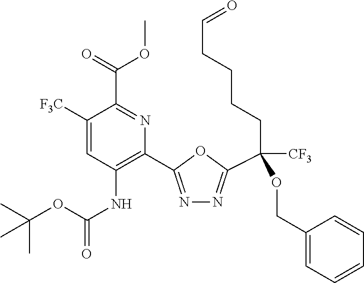 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 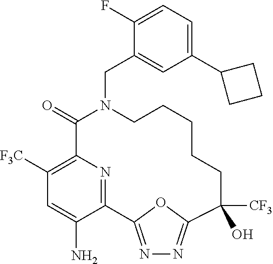 [SMILES: Nc1cc(C(F)(F)F)c2nc1-c1nnc(o1)[C@@](O)(C(F)(F)F)CCCCCN(Cc1cc(C3CCC3)ccc1F)C2=O] | (6R)-17-amino-12- [(5-cyclobutyl-2- fluoro- phenyl)methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 260) |
| | Method 4 | | | |
| | ESI-MS m/z | | | |
| | calc. | | | |
| | 179.11102, | | | |
| | found 180.3 | | | |
| | (M + 1)⁺ | | | |
| 225 | 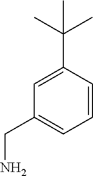 | 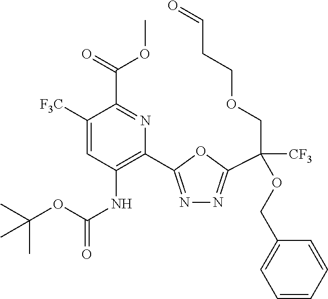 [SMILES: COC(=O)c1nc(-c2nnc(C(COCCC=O)(OCc3ccccc3)C(F)(F)F)o2)c(NC(=O)OC(C)(C)C)cc1C(F)(F)F] | 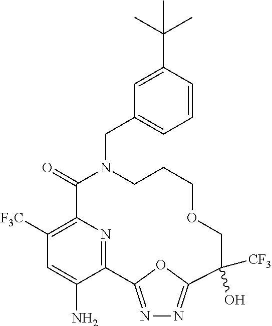 [SMILES: CC(C)(C)c1cccc(CN2CCCOCC(O)(C(F)(F)F)c3nnc(o3)-c3nc(c(C(F)(F)F)cc3N)C2=O)c1] | 17-amino-12-[(3-tert- butylphenyl)methyl]- 6-hydroxy-6,15- bis(trifluoromethyl)- 8,19-dioxa-3,4,12,18- tetrazatricyclo [12.3.1.12,5] nonadeca- 1(18),2,4,14,16- pentaen-13-one (enantiomer 1) (Compound 261) |
| | | | enantiomer 1 | |
| 226 | 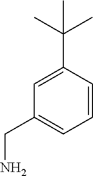 | 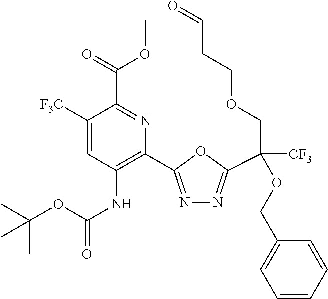 [SMILES: COC(=O)c1nc(-c2nnc(C(COCCC=O)(OCc3ccccc3)C(F)(F)F)o2)c(NC(=O)OC(C)(C)C)cc1C(F)(F)F] | 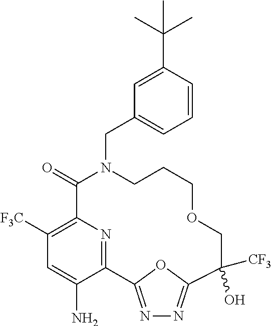 [SMILES: CC(C)(C)c1cccc(CN2CCCOCC(O)(C(F)(F)F)c3nnc(o3)-c3nc(c(C(F)(F)F)cc3N)C2=O)c1] | 17-amino-12-[(3-tert- butylphenyl)methyl]- 6-hydroxy-6,15- bis(trifluoromethyl)- 8,19-dioxa-3,4,12,18- tetrazatricyclo [12.3.1.12,5] nonadeca- 1(18),2,4,14,16- pentaen-13-one (enantiomer 2) (Compound 262) |
| | | | enantiomer 2 | |
| 227 | 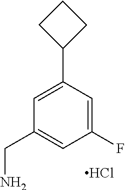 [SMILES: NCc1cc(F)cc(C2CCC2)c1 ; Cl] | 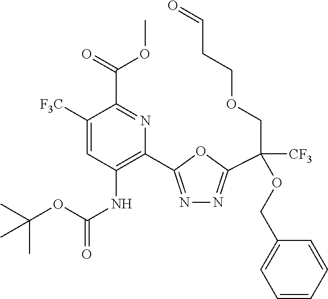 [SMILES: COC(=O)c1nc(-c2nnc(C(COCCC=O)(OCc3ccccc3)C(F)(F)F)o2)c(NC(=O)OC(C)(C)C)cc1C(F)(F)F] | 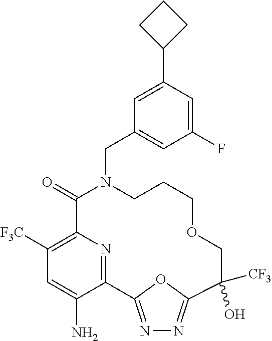 [SMILES: Nc1cc(C(F)(F)F)c2nc1-c1nnc(o1)C(O)(C(F)(F)F)COCCCN(Cc1cc(F)cc(C3CCC3)c1)C2=O] | 17-amino-12-[(3- cyclobutyl-5-fluoro- phenyl)methyl]-6- hydroxy-6,15- bis(trifluoromethyl)- 8,19-dioxa-3,4,12,18- tetrazatricyclo [12.3.1.12,5] nonadeca- 1(18),2,4,14,16- pentaen-13-one (enantiomer 1) (Compound 263) |
| | | | enantiomer 1 | |
| 228 | 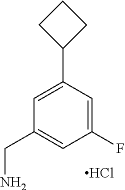 [SMILES: NCc1cc(F)cc(C2CCC2)c1 ; Cl] | 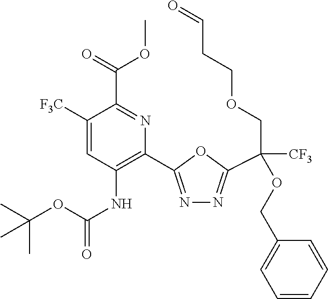 [SMILES: COC(=O)c1nc(-c2nnc(C(COCCC=O)(OCc3ccccc3)C(F)(F)F)o2)c(NC(=O)OC(C)(C)C)cc1C(F)(F)F] | 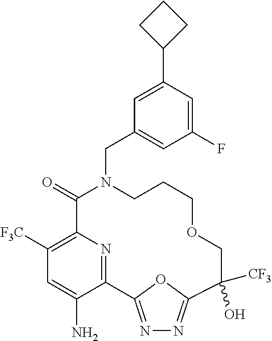 [SMILES: Nc1cc(C(F)(F)F)c2nc1-c1nnc(o1)C(O)(C(F)(F)F)COCCCN(Cc1cc(F)cc(C3CCC3)c1)C2=O] | 17-amino-12-[(3- cyclobutyl-5-fluoro- phenyl)methyl]-6- hydroxy-6,15- bis(trifluoromethyl)- 8,19-dioxa-3,4,12,18- tetrazatricyclo [12.3.1.12,5] nonadeca- 1(18),2,4,14,16- pentaen-13-one (enantiomer 2) (Compound 264) |
| | | | enantiomer 2 | |
| 229 | 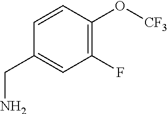 [SMILES: NCc1ccc(OC(F)(F)F)c(F)c1] | 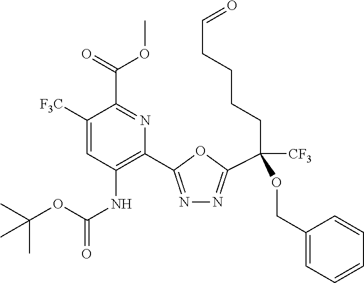 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 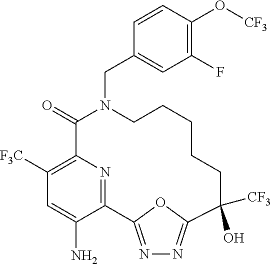 [SMILES: Nc1cc(C(F)(F)F)c2nc1-c1nnc(o1)[C@@](O)(C(F)(F)F)CCCCCN(Cc1ccc(OC(F)(F)F)c(F)c1)C2=O] | (6R)-17-amino-12- [[3-fluoro-4- (trifluoromethoxy) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 265) |
| 230 | 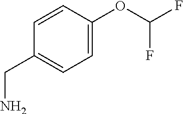 [SMILES: NCc1ccc(OC(F)F)cc1] | 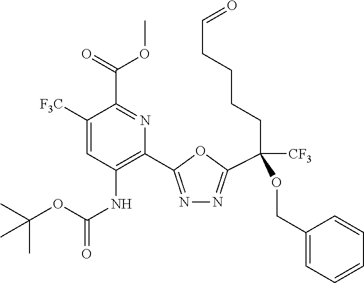 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 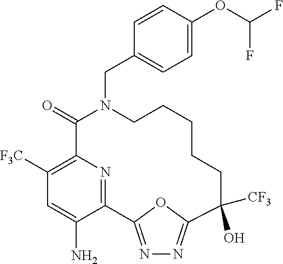 [SMILES: Nc1cc(C(F)(F)F)c2nc1-c1nnc(o1)[C@@](O)(C(F)(F)F)CCCCCN(Cc1ccc(OC(F)F)cc1)C2=O] | (6R)-17-amino-12- [[4- (difluoromethoxy) phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 266) |
| 231 | 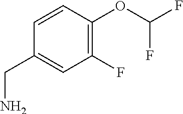 [SMILES: NCc1ccc(OC(F)F)c(F)c1] | 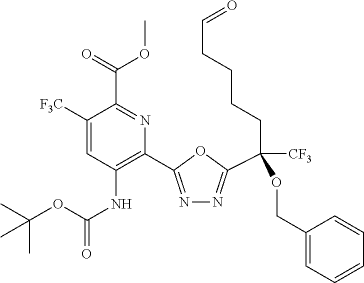 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 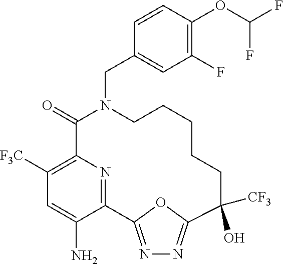 [SMILES: Nc1cc(C(F)(F)F)c2nc1-c1nnc(o1)[C@@](O)(C(F)(F)F)CCCCCN(Cc1ccc(OC(F)F)c(F)c1)C2=O] | (6R)-17-amino-12- [[4- (difluoromethoxy)-3- fluoro- phenyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 267) |
| 232 | 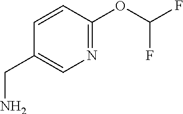 [SMILES: NCc1ccc(OC(F)F)nc1] | 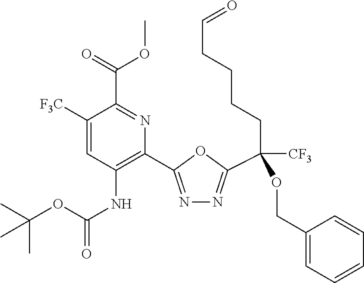 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 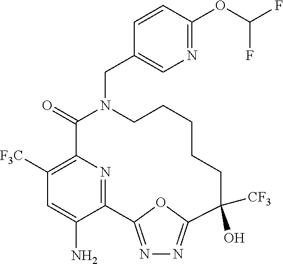 [SMILES: Nc1cc(C(F)(F)F)c2nc1-c1nnc(o1)[C@@](O)(C(F)(F)F)CCCCCN(Cc1ccc(OC(F)F)nc1)C2=O] | (6R)-17-amino-12- [[6- (difluoromethoxy)-3- pyridyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 268) |
| 233 | 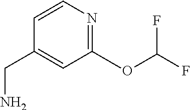 | 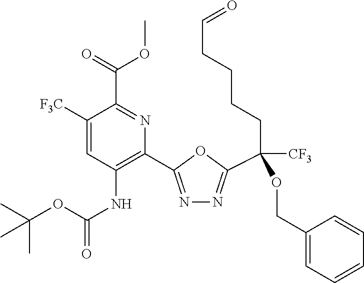 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 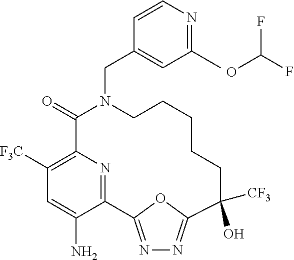 | (6R)-17-amino-12- [[2- (difluoromethoxy)-4- pyridyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 269) |
| 234 | 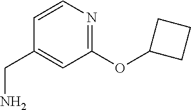 [SMILES: NCc1ccnc(OC2CCC2)c1] | 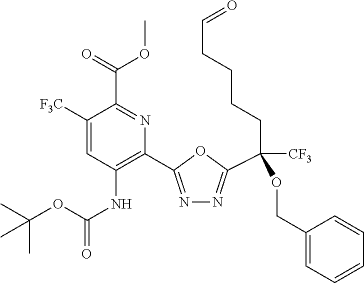 [SMILES: COC(=O)c1nc(-c2nnc([C@@](CCCCC=O)(OCc3ccccc3)C(F)(F)F)o2)c(NC(=O)OC(C)(C)C)cc1C(F)(F)F] | 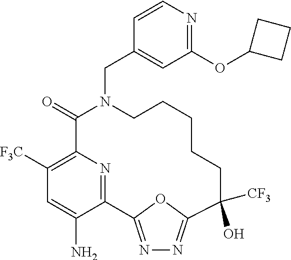 [SMILES: Nc1cc(C(F)(F)F)c2nc1-c1nnc(o1)[C@@](O)(C(F)(F)F)CCCCCN(Cc1ccnc(OC3CCC3)c1)C2=O] | (6R)-17-amino-12- [[2-(cyclobutoxy)-4- pyridyl]methyl]-6- hydroxy-6,15- bis(trifluoromethyl)- 19-oxa-3,4,12,18- tetrazatricyclo [12.3.1.12,5] nonadeca- 1(18),2,4,14,16- pentaen-13-one (Compound 270) |

*utilizing step 1 and the first part of step 2 of Method 1 (N-Boc protection, purification and subsequent N-Boc deprotection described in part 2 of step 2 and step 3 of Method 1 was not performed on this amine)

TABLE 6
Characterization Data and chiral SFC Conditions (if applicable) for Final Compounds Prepared by Method 14

| | | | SFC conditions to |
|---|---|---|---|
| Example | LCMS data and | | separate enantiomers from |
| number | method | NMR data (shifts in ppm) | racemic final product |
| 164 | ESI-MS m/z calc. | ¹H NMR (500 MHz, DMSO-d₆) δ 7.80 (s, | |
| | 529.136, found | 1H), 7.70 (s, 1H), 7.23 (s, 2H), 4.26-4.02 | |
| | 530.2 (M + 1)⁺; | (m, 1H), 3.28-3.10 (m, 2H), 3.10-2.83 (m, | |
| | Retention time: | 4H), 2.30-2.08 (m, 3H), 1.76 (s, 1H), 1.66 | |
| | 2.57 minutes (LC | (d, J = 7.0 Hz, 1H), 1.55 (d, J = 15.0 Hz, | |
| | Method H) | 1H), 1.15-1.02 (m, 1H), 0.90 (s, 1H). | |
| 165 | ESI-MS m/z calc. | ¹H NMR (500 MHz, DMSO-d₆) δ 8.58 (s, | |
| | 595.1266, found | 1H), 7.84 (s, 1H), 7.38 (s, 2H), 7.27 (d, J = | |
| | 596.2 (M + 1)⁺; | 4.2 Hz, 1H), 7.21-7.11 (m, 2H), 4.12-3.90 | |
| | Retention time: | (m, 1H), 3.28-3.15 (m, 2H), 3.11-2.77 (m, | |
| | 2.64 minutes (LC | 4H), 2.46-2.26 (m, 2H), 1.97-1.62 (m, 2H). | |
| | Method H) | | |
| 166 | ESI-MS m/z calc. | | Chiral SFC using a IG |
| | 595.1266, found | | column (250 × 21.2 |
| | 596.2 (M + 1)⁺; | | mm, 5 μm particle size) |
| | Retention time: | | and a gradient of 18% |
| | 1.11 minutes (LC | | MeOH (5 mM NH₃) |
| | Method J) | | and 82% CO₂) over 6 |
| | | | minutes using a flow |
| | | | rate of 70 mL/min. First |
| | | | enantiomer to elute |
| 167 | ESI-MS m/z calc. | | Chiral SFC using a IG |
| | 595.1266, found | | column (250 × 21.2 |
| | 596.1 (M + 1)⁺; | | mm, 5 μm particle size) |
| | Retention time: | | and a gradient of 18% |
| | 1.11 minutes (LC | | MeOH (5 mM NH₃) |
| | Method J) | | and 82% CO₂) over 6 |
| | | | minutes using a flow |
| | | | rate of 70 mL/min. |
| | | | Second enantiomer to |
| | | | elute |
| 168 | ESI-MS m/z calc. | | Chiral SFC using a IA |
| | 481.15485, found | | column (250 × 10 mm, |
| | 482.2 (M + 1)⁺; | | 5 μm particle size) and a |
| | Retention time: | | gradient of 11% |
| | 1.63 minutes (LC | | MeOH (5 mM NH₃) |
| | Method A) | | and 89% CO₂) over 10 |
| | | | minutes using a flow |
| | | | rate of 10 mL/min. First |
| | | | enantiomer to elute |
| 169 | ESI-MS m/z calc. | | Chiral SFC using a IA |
| | 481.15485, found | | column (250 × 10 mm, |
| | 482.3 (M + 1)⁺; | | 5 μm particle size) and a |
| | Retention time: | | gradient of 11% |
| | 1.63 minutes (LC | | MeOH (5 mM NH₃) |
| | Method A) | | and 89% CO₂) over 10 |
| | | | minutes using a flow |
| | | | rate of 10 mL/min. |
| | | | Second enantiomer to |
| | | | elute |
| 170 | ESI-MS m/z calc. | | Chiral SFC using a IA |
| | 521.11096, found | | column (250 × 10 mm, |
| | 522.1 (M + 1)⁺; | | 5 μm particle size) and a |
| | Retention time: | | gradient of 11% |
| | 1.67 minutes (LC | | MeOH (5 mM NH₃) |
| | Method A) | | and 89% CO₂ over 10 |
| | | | minutes using a flow |
| | | | rate of 10 mL/min. First |
| | | | enantiomer to elute |
| 171 | ESI-MS m/z calc. | | Chiral SFC using a IA |
| | 521.11096, found | | column (250 × 10 mm, |
| | 522.2 (M + 1)⁺; | | 5 μm particle size) and a |
| | Retention time: | | gradient of 11% |
| | 1.68 minutes (LC | | MeOH (5 mM NH₃) |
| | Method A) | | and 89% CO₂ over 10 |
| | | | minutes using a flow |
| | | | rate of 10 mL/min. |
| | | | Second enantiomer to |
| | | | elute |
| 172 | ESI-MS m/z calc. | ¹H NMR (400 MHz, methanol-d₄) δ 7.70 (s, | |
| | 589.1735, found | 1H), 4.10 (q, J = 7.1 Hz, 1H), 3.26-3.12 (m, | |
| | 590.3 (M + 1)⁺; | 1H), 3.11-2.96 (m, 1H), 2.76-2.60 (m, | |
| | Retention time: | 3H), 2.18-2.07 (m, 3H), 1.98-1.82 (m, | |
| | 1.5 minutes (LC | 5H), 1.81-1.57 (m, 4H), 1.17-1.02 (m, | |
| | Method A) | 1H), 0.62-0.43 (m, 1H); ¹⁹F NMR (376 | |
| | | MHz, methanol-d₄) δ −61.71, −67.84, −81.23. | |
| 173 | ESI-MS m/z calc. | ¹H NMR (400 MHz, methanol-d₄) δ 7.68 (s, | |
| | 589.1735, found | 1H), 3.25-3.03 (m, 2H), 2.81 (t, J = 13.7 | |
| | 590.2 (M + 1)⁺; | Hz, 1H), 2.70-2.57 (m, 1H), 2.42-2.07 (m, | |
| | Retention time: | 6H), 1.86 (s, 1H), 1.79-1.71 (m, 4H), 1.58 | |
| | 1.54 minutes (LC | (d, J = 3.3 Hz, 2H), 1.31 (d, J = 20.3 Hz, 1H), | |
| | Method A) | 1.08 (d, J = 7.5 Hz, 1H), 0.93-0.81 (m, 1H) | |
| 174 | ESI-MS m/z calc. | ¹H NMR (400 MHz, methanol-d₄) δ 7.72 (s, | |
| | 547.2018, found | 1H), 4.00 (m, 1H), 3.22-2.96 (m, 2H), 2.46 | |
| | 548.2 (M + 1)⁺; | (s, 2H), 2.33-2.14 (m, 2H), 2.11-2.03 (m, | |
| | Retention time: | 2H), 2.01 (s, 2H), 1.96-1.80 (m, 6H), 1.67 | |
| | 1.63 minutes (LC | (s, 2H), 1.57-1.41 (m, 2H), 1.16-1.03 (m, | |
| | Method A) | 1H), 0.76 (s, 1H); | |
| 175 | ESI-MS m/z calc. | ¹H NMR (400 MHz, methanol-d₄) δ 7.70 (s, | |
| | 563.1967, found | 1H), 3.34 (s, 3H), 3.25-2.99 (m, 2H), 2.67 | |
| | 564.3 (M + 1)⁺; | (d, J = 10.4 Hz, 4H), 2.19-2.06 (m, 3H), | |
| | Retention time: | 1.91 (d, J = 11.5 Hz, 6H), 1.74 (d, J = 11.7 | |
| | 1.3 minutes (LC | Hz, 5H); | |
| | Method A) | | |
| 176 | ESI-MS m/z calc. | ¹H NMR (400 MHz, DMSO-d₆) δ 7.82 (s, | |
| | 543.17053, found | 1H), 7.73 (s, 1H), 7.28 (s, 2H), 7.22 (t, J = | |
| | 544.2 (M + 1)⁺; | 7.5 Hz, 1H), 7.11-7.06 (m, 2H), 4.88 (d, J = | |
| | Retention time: | 15.1 Hz, 1H), 4.46 (d, J = 15.0 Hz, 1H), 3.10 | |
| | 1.89 minutes (LC | (t, J = 12.7 Hz, 1H), 2.94 (t, J = 11.8 Hz, 1H), | |
| | Method A) | 2.27 (s, 5H), 2.21-2.12 (m, 1H), 1.97-1.87 | |
| | | (m, 1H), 1.73-1.63 (m, 1H), 1.59-1.49 | |
| | | (m, 1H), 1.08-0.99 (m, 1H), 0.86-0.75 (m, 1H) | |
| 177 | ESI-MS m/z calc. | ¹H NMR (400 MHz, DMSO-d₆) δ 7.79 (s, | |
| | 523.2018, found | 1H), 7.73 (s, 1H), 7.23 (s, 2H), 3.54-3.42 | |
| | 524.3 (M + 1)⁺; | (m, 1H), 3.32-3.25 (m, 1H), 3.20-3.10 (m, | |
| | Retention time: | 1H), 3.08-3.01 (m, 1H), 2.32-2.25 (m, | |
| | 1.98 minutes (LC | 1H), 2.24-2.15 (m, 2H), 1.91-1.83 (m, 1H), | |
| | Method A) | 1.72-1.63 (m, 1H), 1.61-1.53 (m, 1H), | |
| | | 1.47 (t, J = 8.4 Hz, 2H), 1.13-1.06 (m, 1H), | |
| | | 0.94 (s, 9H), 0.87-0.79 (m, 1H). | |
| 178 | ESI-MS m/z calc. | ¹H NMR (400 MHz, DMSO-d₆) δ 8.64 (s, | |
| | 638.16876, found | 1H), 7.95 (d, J = 8.0 Hz, 1H), 7.84 (s, 1H), | |
| | 639.3 (M + 1)⁺; | 7.74 (s, 1H), 7.31 (d, J = 8.9 Hz, 2H), 4.90 | |
| | Retention time: | (d, J = 16.0 Hz, 1H), 4.68 (d, J = 16.0 Hz, | |
| | 1.67 minutes (LC | 1H), 3.34 (t, J = 12.6 Hz, 1H), 3.22-3.10 | |
| | Method A) | (m, 1H), 2.34-2.24 (m, 2H), 2.23-2.11 (m, | |
| | | 1H), 1.99-1.84 (m, 1H), 1.71-1.61 (m, | |
| | | 1H), 1.58-1.48 (m, 1H), 1.43-1.32 (m, | |
| | | 2H), 1.24-1.16 (m, 2H), 1.16-1.05 (m, | |
| | | 1H), 0.93-0.75 (m, 1H). | |
| 179 | ESI-MS m/z calc. | ¹H NMR (400 MHz, DMSO-d₆) δ 7.83 (s, | |
| | 639.1892, found | 1H), 7.74 (s, 1H), 7.51 (d, J = 8.0 Hz, 2H), | |
| | 640.4 (M + 1)⁺; | 7.31 (d, J = 8.3 Hz, 4H), 4.90 (d, J = 15.3 | |
| | Retention time: | Hz, 1H), 4.54 (d, J = 15.3 Hz, 1H), 3.14 (t, | |
| | 2.07 minutes (LC | J = 12.6 Hz, 1H), 3.04-2.94 (m, 1H), 2.34- | |
| | Method A) | 2.25 (m, 2H), 2.19-2.13 (m, 1H), 1.99- | |
| | | 1.81 (m, 1H), 1.78-1.63 (m, 1H), 1.54 (s, | |
| | | 7H), 1.10-1.00 (m, 1H), 0.93-0.75 (m, 1H). | |
| 180 | ESI-MS m/z calc. | ¹H NMR (400 MHz, methanol-d₄) δ 7.76 (s, | |
| | 607.06537, found | 1H), 7.53 (d, J = 2.2 Hz, 1H), 7.43 (d, J = 8.0 | |
| | 608.01 (M + 1)⁺; | Hz, 1H), 7.32 (d, J = 7.6 Hz, 1H), 7.26 (t, J = | |
| | Retention time: | 7.7 Hz, 1H), 5.03 (d, J = 15.2 Hz, 1H), 4.57 | |
| | 1.9 minutes (LC | (d, J = 15.1 Hz, 1H), 3.27-3.13 (m, 1H), | |
| | Method N) | 3.06 (m,\1H), 2.54-2.36 (m, 2H), 2.29- | |
| | | 2.18 (m, 1H), 1.95 (dd, J = 11.0, 5.7 Hz, 1H), | |
| | | 1.87-1.73 (m, 1H), 1.66 (s, 1H), 1.16 (m, | |
| | | 1H), 0.87 (m, 1H) | |
| 181 | ESI-MS m/z calc. | ¹H NMR (400 MHz, methanol-d₄) δ 7.75 (s, | |
| | 569.18616, found | 1H), 7.21 (d, J = 7.9 Hz, 2H), 7.04 (d, J = 8.0 | |
| | 570.17 (M + 1)⁺; | Hz, 2H), 5.04 (d, J = 14.9 Hz, 1H), 4.49 (d, | |
| | Retention time: | J = 14.9 Hz, 1H), 3.17 (d, J = 11.7 Hz, 1H), | |
| | 1.94 minutes (LC | 3.08-2.96 (m, 1H), 2.48-2.34 (m, 2H), | |
| | Method A) | 2.29-2.19 (m, 1H), 1.91 (m, 2H), 1.83- | |
| | | 1.74 (m, 1H), 1.64 (s, 1H), 1.16-1.07 (m, | |
| | | 1H), 0.97-0.91 (m, 2H), 0.89-0.80 (m, | |
| | | 1H), 0.68-0.62 (m, 2H). | |
| 182 | ESI-MS m/z calc. | ¹H NMR (400 MHz, methanol-d₄) δ 7.70 (s, | |
| | 521.18616, found | 1H), 3.64-3.41 (m, 1H), 2.88 (t, J = 13.6 | |
| | 522.2 (M + 1)⁺; | Hz, 1H), 2.77-2.55 (m, 2H), 2.47 (s, 1H), | |
| | Retention time: | 2.22-2.05 (m, 1H), 1.99-1.57 (m, 4H), | |
| | 1.48 minutes (LC | 1.54-1.45 (m, 3H), 1.40 (s, 3H), 1.12-0.79 | |
| | Method A) | (m, 1H), 0.54 (d, J = 8.8 Hz, 4H); | |
| 183 | ESI-MS m/z calc. | ¹H NMR (400 MHz, methanol-d₄) δ 7.69 (s, | |
| | 535.2018, found | 1H), 3.24-3.08 (m, 1H), 2.89 (t, J = 13.8 | |
| | 536.3 (M + 1)⁺; | Hz, 1H), 2.78-2.59 (m, 2H), 2.54-2.28 (m, | |
| | Retention time: | 2H), 2.19-1.83 (m, 4H), 1.70-1.51 (m, | |
| | 1.6 minutes (LC | 9H), 1.49-1.30 (m, 1H), 1.15-1.01 (m, | |
| | Method A) | 1H), 0.94-0.79 (m, 1H), 0.43 (s, 1H); | |
| 184 | ESI-MS m/z calc. | ¹H NMR (400 MHz, DMSO-d₆) δ 7.83 (s, | |
| | 637.1718, found | 1H), 7.73 (s, 1H), 7.41 (s, 1H), 7.36 (d, J = | |
| | 638.2 (M + 1)⁺; | 4.5 Hz, 2H), 7.34-7.24 (m, 3H), 4.87 (d, J = | |
| | Retention time: | 15.2 Hz, 1H), 4.58 (d, J = 15.3 Hz, 1H), 3.15 | |
| | 2.04 minutes (LC | (t, J = 12.6 Hz, 1H), 2.95 (t, J = 17.7 Hz, | |
| | Method A) | 1H), 2.34-2.23 (m, 2H), 2.21-2.12 (m, | |
| | | 1H), 1.94-1.82 (m, 1H), 1.74-1.64 (m, | |
| | | 1H), 1.55-1.48 (m, 1H), 1.37-1.28 (m, | |
| | | 2H), 1.12-1.06 (m, 2H), 0.89-0.72 (m, 2H). | |
| 185 | ESI-MS m/z calc. | ¹H NMR (400 MHz, DMSO-d₆) δ 7.83 (s, | |
| | 585.2218, found | 1H), 7.75 (s, 1H), 7.35 (s, 1H), 7.33-7.21 | |
| | 586.3 (M + 1)⁺; | (m, 4H), 7.10 (d, J = 7.0 Hz, 1H), 4.88 (d, | |
| | Retention time: | J = 15.1 Hz, 1H), 4.54 (d, J = 15.1 Hz, 1H), | |
| | 2.13 minutes (LC | 3.13 (t, J = 12.5 Hz, 1H), 2.98-2.89 (m, | |
| | Method A) | 1H), 2.35-2.24 (m, 2H), 2.21-2.13 (m, | |
| | | 1H), 1.96-1.83 (m, 1H), 1.73-1.63 (m, | |
| | | 1H), 1.58-1.49 (m, 1H), 1.25 (s, 9H), 1.09- | |
| | | 0.99 (m, 1H), 0.86-0.72 (m, 1H). | |
| 186 | ESI-MS m/z calc. | ¹H NMR (400 MHz, DMSO-d₆) δ 7.83 (s, | |
| | 539.1918, found | 1H), 7.74 (s, 1H), 7.49 (s, 1H), 7.44 (d, J = | |
| | 640.3 (M + 1)⁺; | 7.9 Hz, 1H), 7.37 (t, J = 7.7 Hz, 1H), 7.33- | |
| | Retention time: | 7.23 (m, 3H), 4.88 (d, J = 15.2 Hz, 1H), 4.61 | |
| | 2.06 minutes (LC | (d, J = 15.3 Hz, 1H), 3.16 (t, J = 12.5 Hz, | |
| | Method A) | 1H), 2.98-2.92 (m, 1H), 2.35-2.24 (m, | |
| | | 2H), 2.20-2.11 (m, 1H), 1.91-1.83 (m, | |
| | | 1H), 1.73-1.63 (m, 1H), 1.53 (s, 6H), 1.29- | |
| | | 1.19 (m, 1H), 1.09-0.99 (m, 1H), 0.85- | |
| | | 0.71 (m, 1H). | |
| 187 | ESI-MS m/z calc. | ¹H NMR (400 MHz, DMSO-d₆) δ 7.83 (s, | |
| | 667.1813, found | 1H), 7.74 (s, 1H), 7.36-7.20 (m, 3H), 6.90 | |
| | 668.2 (M + 1)⁺; | (d, J = 8.3 Hz, 2H), 6.84 (d, J = 8.5 Hz, 1H), | |
| | Retention time: | 4.87 (d, J = 15.2 Hz, 1H), 4.49 (d, J = 15.2 | |
| | 2.05 minutes (LC | Hz, 1H), 4.07 (s, 2H), 3.13 (t, J = 12.5 Hz, | |
| | Method A) | 1H), 2.99-2.87 (m, 1H), 2.34-2.24 (m, | |
| | | 2H), 2.23-2.12 (m, 1H), 1.99-1.87 (m, | |
| | | 1H), 1.76-1.63 (m, 1H), 1.61-1.48 (m, | |
| | | 1H), 1.12-1.07 (m, 2H), 1.06-1.01 (m, | |
| | | 1H), 1.01-0.90 (m, 2H), 0.87-0.73 (m, 1H). | |
| 188 | ESI-MS m/z calc. | ¹H NMR (400 MHz, DMSO-d₆) δ 7.83 (s, | |
| | 619.1818, found | 1H), 7.74 (s, 1H), 7.37-7.25 (m, 3H), 7.26- | |
| | 620.2 (M + 1)⁺; | 7.13 (m, 3H), 4.90 (d, J = 15.3 Hz, 1H), 4.53 | |
| | Retention time: | (d, J = 15.3 Hz, 1H), 3.13 (t, J = 12.6 Hz, | |
| | 1.95 minutes (LC | 1H), 2.99-2.91 (m, 2H), 2.72-2.55 (m, | |
| | Method A) | 4H), 2.34-2.24 (m, 2H), 2.21-2.12 (m, | |
| | | 1H), 1.98-1.86 (m, 1H), 1.75-1.64 (m, | |
| | | 1H), 1.60-1.49 (m, 1H), 1.08-0.98 (m, | |
| | | 1H), 0.87-0.76 (m, 1H). | |
| 189 | ESI-MS m/z calc. | ¹H NMR (400 MHz, DMSO-d₆) δ 7.83 (s, | |
| | 557.1918, found | 1H), 7.74 (s, 1H), 7.28 (s, 2H), 6.89 (s, 3H), | |
| | 558.2 (M + 1)⁺; | 4.85 (d, J = 15.1 Hz, 1H), 4.42 (d, J = 15.1 | |
| | Retention time: | Hz, 1H), 3.09 (t, J = 12.5 Hz, 1H), 2.93 (t, | |
| | 2.01 minutes (LC | J = 9.3 Hz, 1H), 2.23 (s, 9H), 2.00-1.84 (m, | |
| | Method A) | 1H), 1.72-1.62 (m, 1H), 1.60-1.47 (m, | |
| | | 1H), 1.10-0.97 (m, 1H), 0.91-0.75 (m, 1H). | |
| 190 | ESI-MS m/z calc. | ¹H NMR (400 MHz, methanol-d₄) δ 7.75- | |
| | 535.2018, found | 7.72 (m, 1H), 3.64-3.45 (m, 1H), 3.18- | |
| | 536.1 (M + 1)⁺; | 3.06 (m, 1H), 2.56 (dd, J = 15.2, 10.9 Hz, | |
| | Retention time: | 1H), 2.34 (q, J = 6.2, 5.5 Hz, 1H), 2.25-2.10 | |
| | 1.53 minutes (LC | (m, 1H), 2.00-1.78 (m, 1H), 1.66 (d, J = | |
| | Method A) | 44.9 Hz, 2H), 1.37-1.21 (m, 11H), 0.90 (m, 4H). | |
| 191 | ESI-MS m/z calc. | ¹H NMR (400 MHz, methanol-d₄) δ 7.74 (s, | Chiral SFC using a AD |
| | 611.2331, found | 1H), 7.32 (d, J = 8.5 Hz, 2H), 7.23 (d, J = 8.4 | column (250 × 21.2 |
| | 612.3 (M + 1)⁺; | Hz, 2H), 3.52-3.35 (m, 2H), 2.47-2.31 (m, | mm, 5 μm particle size) |
| | Retention time: | 2H), 2.23-2.11 (m, 1H), 1.91-1.78 (m, | and a gradient of 10 to |
| | 1.73 minutes (LC | 1H), 1.77-1.67 (m, 1H), 1.62-1.39 (m, | 60% MeOH (5 mM |
| | Method A) | 5H), 1.29 (s, 9H), 1.10 (m, 1H), 0.82 (m, 1H). | NH₃) and CO₂ over 10 |
| | | | minutes using a flow |
| | | | rate of 10 mL/min. First |
| | | | enantiomer to elute |
| 192 | ESI-MS m/z calc. | ¹H NMR (400 MHz, methanol-d₄) δ 7.74 (s, | Chiral SFC using a AD |
| | 611.2331, found | 1H), 7.32 (d, J = 8.4 Hz, 2H), 7.23 (d, J = 8.5 | column (250 × 21.2 |
| | 612.4 (M + 1)⁺; | Hz, 2H), 3.56-3.32 (m, 2H), 2.45-2.31 (m, | mm, 5 μm particle size) |
| | Retention time: | 2H), 2.22-2.10 (m, 1H), 1.91-1.79 (m, | and a gradient of 10 to |
| | 1.73 minutes (LC | 1H), 1.75-1.66 (m, 1H), 1.62-1.40 (m, | 60% MeOH (5 mM |
| | Method A) | 5H), 1.29 (s, 9H), 1.18-1.05 (m, 1H), 0.94- | NH₃) and CO₂ over 10 |
| | | 0.72 (m, 1H). | minutes using a flow |
| | | | rate of 10 mL/min. |
| | | | Second enantiomer to |
| | | | elute |
| 193 | ESI-MS m/z calc. | | |
| | 583.2018, found | | |
| | 584.3 (M + 1)⁺; | | |
| | Retention time: | | |
| | 1.67 minutes (LC | | |
| | Method A) | | |
| 194 | ESI-MS m/z calc. | ¹H NMR (400 MHz, DMSO-d₆) δ 7.83 (s, | |
| | 627.1518, found | 1H), 7.74 (s, 1H), 7.59 (d, J = 8.0 Hz, 1H), | |
| | 628.2 (M + 1)⁺; | 7.31 (s, 2H), 7.17 (s, 1H), 7.01 (d, J = 8.0 | |
| | Retention time: | Hz, 1H), 4.94 (d, J = 15.7 Hz, 1H), 4.61 (d, | |
| | 1.95 minutes (LC | J = 15.7 Hz, 1H), 3.85 (s, 3H), 3.21 (t, J = | |
| | Method A) | 12.7 Hz, 1H), 3.08-2.96 (m, 1H), 2.38-2.25 (m, | |
| | | 2H), 2.22 (d, J = 13.6 Hz, 1H), 1.95 (s, 1H), | |
| | | 1.69 (s, 1H), 1.55 (s, 1H), 1.11 (s, 1H), 0.85 | |
| | | (s, 1H). | |
| 195 | ESI-MS m/z calc. | ¹H NMR (400 MHz, methanol-d₄) δ 7.76 (s, | |
| | 569.18616, found | 1H), 7.19 (t, J = 7.6 Hz, 1H), 7.14-6.95 (m, | |
| | 570.4 (M + 1)⁺; | 3H), 5.02 (d, J = 15.5 Hz, 1H), 4.53 (d, J = | |
| | Retention time: | 15.0 Hz, 1H), 3.17 (m, 1H), 3.02 (m, 1H), | |
| | 1.93 minutes (LC | 2.55-2.37 (m, 2H), 2.30-2.15 (m, 1H), | |
| | Method A) | 1.99-1.91 (m, 1H), 1.88 (m, 1H), 1.84- | |
| | | 1.74 (m, 1H), 1.69-1.59 (m, 1H), 1.13 (m, | |
| | | 1H), 0.97-0.89 (m, 2H), 0.85 (m, 1H), 0.66 | |
| | | (m, 2H). | |
| 196 | ESI-MS m/z calc. | ¹H NMR (400 MHz, DMSO-d₆) δ 7.76 (s, | Chiral SFC using a |
| | 481.15485, found | 1H), 7.62 (s, 1H), 7.03 (s, 2H), 3.55-3.37 | LUX-4 column (250 × |
| | 482.0 (M + 1)⁺; | (m, 2H), 3.18-2.95 (m, 2H), 2.42-2.21 (m, | 21.2 mm, 5 μm particle |
| | Retention time: | 3H), 2.07 (t, J = 17.3 Hz, 2H), 1.44 (s, 1H), | size) and a gradient of |
| | 2.32 minutes (LC | 1.29 (s, 1H), 0.89 (d, J = 6.7 Hz, 3H), 0.86 | 10% to 25% MeOH (5 |
| | Method D) | (d, J = 6.6 Hz, 3H). | mM NH₃) and CO₂ |
| | | | over 14.5 minutes using |
| | | | a flow rate of 40 |
| | | | mL/min. First |
| | | | enantiomer to elute |
| 197 | ESI-MS m/z calc. | ¹H NMR (400 MHz, DMSO-d₆) δ 7.76 (s, | Chiral SFC using a |
| | 481.15485, found | 1H), 7.64 (s, 1H), 7.05 (s, 2H), 3.55-3.41 | LUX-4 column (250 × |
| | 482.0 (M + 1)⁺; | (m, 2H), 3.17-3.02 (m, 2H), 2.35 (d, J = | 21.2 mm, 5 μm particle |
| | Retention time: | 15.6 Hz, 2H), 2.28-2.15 (m, 1H), 2.11- | size) and a gradient of |
| | 2.32 minutes (LC | 2.01 (m, 2H), 1.48-1.38 (m, 1H), 1.34- | 10% to 25% MeOH (5 |
| | Method D) | 1.27 (m, 1H), 0.88 (d, J = 6.7 Hz, 3H), 0.85 | mM NH₃) and CO₂ |
| | | (d, J = 6.6 Hz, 3H). | over 14.5 minutes using |
| | | | a flow rate of 40 |
| | | | mL/min. Second |
| | | | enantiomer to elute |
| 198 | ESI-MS m/z calc. | ¹H NMR (400 MHz, DMSO-d₆) δ 7.80 (s, | Chiral SFC using a |
| | 599.1215, found | 1H), 7.66 (s, 1H), 7.41 (d, J = 8.5 Hz, 2H), | LUX-4 column (250 × |
| | 600.0 (M + 1)⁺; | 7.34 (d, J = 8.3 Hz, 2H), 7.13 (s, 2H), 4.88 (d, | 21.2 mm, 5 μm particle |
| | Retention time: | J = 15.5 Hz, 1H), 4.52 (d, J = 15.2 Hz, 1H), | size) and a gradient of |
| | 2.68 minutes (LC | 3.55-3.45 (m, 1H), 3.11-3.03 (m, 1H), 2.46- | 10% to 20% MeOH (5 |
| | Method D) | 2.41 (m, 1H), 2.35-2.24 (m, 2H), 2.07- | mM NH₃) and CO₂ |
| | | 1.99 (m, 1H), 1.47-1.38 (m, 1H), 1.36-1.27 | over 14.5 minutes using |
| | | (m, 1H). | a flow rate of 40 |
| | | | mL/min. First |
| | | | enantiomer to elute |
| 199 | ESI-MS m/z calc. | ¹H NMR (400 MHz, DMSO-d₆) δ 7.80 (s, | Chiral SFC using a |
| | 599.1215, found | 1H), 7.68 (s, 1H), 7.41 (d, J = 8.6 Hz, 2H), | LUX-4 column (250 × |
| | 600.0 (M + 1)⁺; | 7.33 (d, J = 8.3 Hz, 2H), 7.12 (s, 2H), 4.87 | 21.2 mm, 5 μm particle |
| | Retention time: | (d, J = 14.6 Hz, 1H), 4.52 (d, J = 15.0 Hz, | size) and a gradient of |
| | 2.68 minutes (LC | 1H), 3.59-3.43 (m, 1H), 3.15-2.98 (m, | 10% to 20% MeOH (5 |
| | Method D) | 1H), 2.44 (d, J = 3.6 Hz, 1H), 2.37-2.21 (m, | mM NH₃) and CO₂ |
| | | 2H), 2.03 (d, J = 13.8 Hz, 1H), 1.50-1.25 | over 14.5 minutes using |
| | | (m, 2H). | a flow rate of 40 |
| | | | mL/min. Second |
| | | | enantiomer to elute |
| 200 | ESI-MS m/z calc. | ¹H NMR (400 MHz, DMSO-d₆) δ 7.82 (s, | Chiral SFC using a AD |
| | 607.06537, found | 1H), 7.74 (s, 1H), 7.55 (d, J = 8.4 Hz, 2H), | column (250 × 21.2 |
| | 609.96 (M + 3, Br | 7.29 (s, 2H), 7.26 (d, J = 8.2 Hz, 2H), 4.85 | mm, 5 μm particle size) |
| | isotope)⁺; | (d, J = 15.2 Hz, 1H), 4.50 (d, J = 15.2 Hz, | and a gradient of 10% |
| | Retention time: | 1H), 3.17 (d, J = 13.4 Hz, 1H), 3.02-2.90 | to 60% MeOH (5 mM |
| | 1.93 minutes (LC | (m, 1H), 2.30-2.23 (m, 2H), 2.20-2.15 (m, | NH₃) and CO₂ over 10 |
| | Method A) | 1H), 1.96-1.88 (m, 1H), 1.71-1.64 (m, | minutes using a flow |
| | | 1H), 1.57-1.50 (m, 1H), 1.09-1.01 (m, | rate of 60 mL/min. First |
| | | 1H), 0.87-0.79 (m, 1H). | enantiomer to elute |
| 201 | ESI-MS m/z calc. | ¹H NMR (400 MHz, DMSO-d₆) δ 7.82 (s, | Chiral SFC using a AD |
| | 607.06537, found | 1H), 7.75 (s, 1H), 7.55 (d, J = 8.4 Hz, 2H), | column (250 × 21.2 |
| | 610.0 (M + 3, Br | 7.30 (s, 2H), 7.26 (d, J = 8.4 Hz, 2H), 4.85 | mm, 5 μm particle size) |
| | isotope)⁺; | (d, J = 15.2 Hz, 1H), 4.50 (d, J = 15.3 Hz, | and a gradient of 10% |
| | Retention time: | 1H), 3.16 (t, J = 12.7 Hz, 1H), 3.03-2.98 | to 60% MeOH (5 mM |
| | 1.92 minutes (LC | (m, 1H), 2.35-2.19 (m, 2H), 2.20-2.10 (m, | NH₃) and CO₂ over 10 |
| | Method A) | 1H), 1.95-1.88 (m, 1H), 1.72-1.63 (m, | minutes using a flow |
| | | 1H), 1.59-1.50 (m, 1H), 1.10-1.00 (m, | rate of 60 mL/min. |
| | | 1H), 0.88-0.79 (m, 1H). | Second enantiomer to |
| | | | elute |
| 202 | ESI-MS m/z calc. | ¹H NMR (400 MHz, DMSO-d₆) δ 7.80 (s, | Chiral SFC using a |
| | 515.1392, found | 1H), 7.61 (s, 1H), 7.29 (m, 5H), 7.10 (s, 2H), | LUX-4 column (250 × |
| | 516.0 (M + 1)⁺; | 4.90 (d, J = 11.1 Hz, 1H), 4.47 (d, J = 15.9 | 21.2 mm, 5 μm particle |
| | Retention time: | Hz, 1H), 3.46 (s, 1H), 3.11-2.98 (m, 1H), | size) and a gradient of |
| | 2.35 minutes (LC | 2.36 (dd, J = 50.0, 9.2 Hz, 3H), 2.03 (d, J = | 20% to 25% MeOH (5 |
| | Method D) | 15.7 Hz, 1H), 1.54-1.20 (m, 2H). | mM NH₃) and CO₂ |
| | | | over 14.5 minutes using |
| | | | a flow rate of 40 |
| | | | mL/min. First |
| | | | enantiomer to elute |
| 203 | ESI-MS m/z calc. | ¹H NMR (400 MHz, DMSO-d₆) δ 7.80 (s, | Chiral SFC using a |
| | 515.1392, found | 1H), 7.62 (s, 1H), 7.29 (m, 5H), 7.10 (s, 2H), | LUX-4 column (250 × |
| | 516.0 (M + 1)⁺; | 4.90 (d, J = 14.4 Hz, 1H), 4.47 (d, J = 13.0 | 21.2 mm, 5 μm particle |
| | Retention time: | Hz, 1H), 3.43 (d, J = 11.8 Hz, 1H), 3.03 (d, | size) and a gradient of |
| | 2.35 minutes (LC | J = 3.9 Hz, 1H), 2.36 (dd, J = 51.9, 8.7 Hz, | 20% to 25% MeOH (5 |
| | Method D) | 3H), 2.10-1.94 (m, 1H), 1.51-1.18 (m, 2H). | mM NH₃) and CO₂ |
| | | | over 14.5 minutes using |
| | | | a flow rate of 40 |
| | | | mL/min. Second |
| | | | enantiomer to elute |
| 204 | ESI-MS m/z calc. | | |
| | 595.2018, found | | |
| | 596.2 (M + 1)⁺; | | |
| | Retention time: | | |
| | 2.14 minutes (LC | | |
| | Method A) | | |
| 205 | ESI-MS m/z calc. | ¹H NMR (400 MHz, methanol-d₄) δ 7.76 (s, | |
| | 579.1517, found | 1H), 7.61-7.33 (m, 4H), 6.73 (t, J = 56.3 | |
| | 580.2 (M + 1)⁺; | Hz, 1H), 5.09 (d, J = 15.3 Hz, 1H), 4.65 (d, | |
| | Retention time: | J = 15.3 Hz, 1H), 3.26-3.19 (m, 1H), 3.14- | |
| | 0.7 minutes (LC | 3.01 (m, 1H), 2.51-2.39 (m, 2H), 2.30- | |
| | Method S) | 2.17 (m, 1H), 2.02-1.89 (m, 1H), 1.86- | |
| | | 1.74 (m, 1H), 1.65 (s, 1H), 1.14 (m, 1H), | |
| | | 0.92-0.82 (m, 1H). | |
| 206 | ESI-MS m/z calc. | ¹H NMR (400 MHz, methanol-d₄) δ 7.73 (s, | |
| | 571.183, found | 1H), 3.67 (dd, J = 13.5, 7.7 Hz, 1H), 3.28- | |
| | 572.19 (M + 1)⁺; | 3.01 (m, 3H), 2.51-2.30 (m, 2H), 2.25 (m, | |
| | Retention time: | 1H), 2.14-1.88 (m, 4H), 1.88-1.47 (m, | |
| | 1.79 minutes (LC | 6H), 1.48-0.98 (m, 5H), 0.99-0.74 (m, 2H | |
| | Method A) | | |
| 207 | ESI-MS m/z calc. | ¹H NMR (400 MHz, methanol-d₄) δ 7.73 (s, | |
| | 543.1517, found | 1H), 3.88 (dd, J = 13.8, 6.4 Hz, 1H), 3.58- | |
| | 544.13 (M + 1)⁺; | 3.41 (m, 1H), 3.23-3.01 (m, 2H), 2.62 (m, | |
| | Retention time: | 3H), 2.53-2.31 (m, 4H), 2.25 (m, 1H), 1.94 | |
| | 1.69 minutes (LC | (qt, J = 10.8, 5.2 Hz, 1H), 1.81 (m, 1H), 1.74- | |
| | Method A) | 1.52 (m, 2H), 1.19 (m, 2H), 1.03-0.70 (m, | |
| | | 2H). | |
| 208 | ESI-MS m/z calc. | ¹H NMR (400 MHz, DMSO-d₆) δ 7.82 (s, | |
| | 587.2017, found | 1H), 7.74 (s, 1H), 7.38 (d, J = 8.1 Hz, 2H), | |
| | 588.18 (M + 1)⁺; | 7.27 (d, J = 7.2 Hz, 3H), 7.11 (d, J = 7.5 Hz, | |
| | Retention time: | 1H), 4.98 (s, 1H), 4.89 (d, J = 15.1 Hz, 1H), | |
| | 1.61 minutes (LC | 4.53 (d, J = 15.0 Hz, 1H), 3.13 (t, J = 12.5 | |
| | Method A) | Hz, 1H), 3.02-2.92 (m, 1H), 2.34-2.23 (m, | |
| | | 2H), 2.22-2.14 (m, 1H), 1.96-1.86 (m, | |
| | | 1H), 1.73-1.63 (m, 1H), 1.58-1.46 (m, | |
| | | 1H), 1.39 (d, J = 3.3 Hz, 6H), 1.09-0.96 (m, | |
| | | 1H), 0.86-0.74 (m, 1H). | |
| 209 | ESI-MS m/z calc. | ¹H NMR (400 MHz, DMSO-d₆) δ 7.79 (s, | |
| | 545.19, found | 1H), 7.73 (s, 1H), 7.23 (s, 2H), 3.72-3.62 | |
| | 546.30 (M + 1)⁺; | (m, 1H), 3.45-3.37 (m, 2H), 3.18-3.09 (m, | |
| | Retention time: | 1H), 3.05-2.96 (m, 1H), 2.32-2.23 (m, | |
| | 1.99 minutes (LC | 1H), 2.22-2.11 (m, 2H), 1.89-1.78 (m, | |
| | Method A) | 1H), 1.69 (t, J = 6.3 Hz, 2H), 1.60-1.50 (m, | |
| | | 1H), 1.15-1.04 (m, 1H), 0.95-0.76 (m, | |
| | | 4H), 0.73-0.59 (m, 2H), 0.61-0.48 (m, 2H). | |
| 210 | ESI-MS m/z calc. | ¹H NMR (400 MHz, DMSO-d₆) δ 7.79 (s, | |
| | 587.2001, found | 1H), 7.73 (s, 1H), 7.22 (s, 2H), 3.66-3.38 | |
| | 588.18 (M + 1)⁺; | (m, 2H), 3.18 (t, J = 12.4 Hz, 2H), 2.31- | |
| | Retention time: | 2.23 (m, 1H), 2.22-2.12 (m, 2H), 1.96- | |
| | 2.04 minutes (LC | 1.75 (m, 4H), 1.73-1.63 (m, 2H), 1.61- | |
| | Method A) | 1.50 (m, 3H), 1.19-1.03 (m, 4H), 0.91- | |
| | | 0.78 (m, 3H). | |
| 211 | ESI-MS m/z calc. | ¹H NMR (400 MHz, methanol-d₄) δ 7.76 (s, | |
| | 587.1767, found | 1H), 6.88 (d, J = 1.6 Hz, 1H), 6.85-6.78 (m, | |
| | 588.3 (M + 1)⁺; | 1H), 6.71 (m, 1H), 4.99 (d, J = 15.3 Hz, 1H), | |
| | Retention time: | 4.53 (d, J = 15.3 Hz, 1H), 3.26-3.15 (m, | |
| | 1.57 minutes (LC | 1H), 3.04 (m, 1H), 2.45 (m, 2H), 2.23 (m, | |
| | Method A) | 1H), 1.99-1.92 (m, 1H), 1.89 (m, 1H), 1.79 | |
| | | (m, 1H), 1.70-1.57 (m, 1H), 1.14 (m, 1H), | |
| | | 1.02-0.95 (m, 2H), 0.91-0.83 (m, 1H), | |
| | | 0.73-0.64 (m, 2H). ¹⁹F NMR (376 MHz, | |
| | | methanol-d₄) δ −61.37, −81.20, −116.29. | |
| 212 | ESI-MS m/z calc. | ¹H NMR (400 MHz, CDCl₃) δ 7.67 (s, 1H), | |
| | 601.1924, found | 6.93 (s, 1H), 6.85-6.77 (m, 2H), 5.14 (d, | |
| | 602.3 (M + 1)⁺; | J = 15.3 Hz, 1H), 4.43 (d, J = 15.3 Hz, 1H), | |
| | Retention time: | 3.56-3.45 (m, 1H), 3.26-3.17 (m, 1H), | |
| | 1.69 minutes (LC | 3.09-2.95 (m, 1H), 2.55-2.41 (m, 2H), | |
| | Method A) | 2.39-2.19 (m, 3H), 2.17-1.99 (m, 3H), | |
| | | 1.98-1.88 (m, 1H), 1.88-1.75 (m, 2H), | |
| | | 1.73-1.55 (m, 1H), 1.24-1.11 (m, 1H), | |
| | | 0.93-0.78 (m, 1H).. ¹⁹F NMR (376 MHz, | |
| | | Chloroform-d) δ −56.55, −76.26, −110.45. | |
| 213 | ESI-MS m/z calc. | ¹H NMR (400 MHz, DMSO-d₆) δ 7.79 (d, | Chiral SFC using a |
| | 425.09225, found | J = 8.6 Hz, 2H), 7.59 (s, 1H), 7.07 (s, 2H), | LUX-4 column (250 × |
| | 426.0 (M + 1)⁺; | 3.22 (dd, J = 24.8, 12.4 Hz, 1H), 3.04-2.87 | 21.2 mm, 5 μm particle |
| | Retention time: | (m, 1H), 2.40-2.26 (m, 2H), 2.11-1.97 (m, | size) and a gradient of |
| | 1.48 minutes (LC | 2H), 1.47 (s, 1H), 1.40-1.27 (m, 1H). ¹⁹F | 15% to 25% MeOH (5 |
| | Method D) | NMR (376 MHz, DMSO-d₆) δ −58.83, −78.02. | mM NH₃) and CO₂ |
| | | | over 14.5 minutes using |
| | | | a flow rate of 40 |
| | | | mL/min. First |
| | | | enantiomer to elute |
| 214 | ESI-MS m/z calc. | ¹H NMR (400 MHz, DMSO-d₆) δ 7.84 (t, J = | Chiral SFC using a |
| | 425.09225, found | 8.1 Hz, 1H), 7.78 (s, 1H), 7.64 (s, 1H), 7.10 | LUX-4 column (250 × |
| | 426.0 (M + 1)⁺; | (s, 2H), 3.32-3.13 (m, 1H), 2.98-2.89 (m, | 21.2 mm, 5 μm particle |
| | Retention time: | 1H), 2.41-2.30 (m, 2H), 2.10-1.96 (m, | size) and a gradient of |
| | 1.48 minutes (LC | 2H), 1.54-1.42 (m, 1H), 1.40-1.28 (m, 1H). | 15% to 25% MeOH (5 |
| | Method D) | | mM NH₃) and CO₂ |
| | | | over 14.5 minutes using |
| | | | a flow rate of 40 |
| | | | mL/min. Second |
| | | | enantiomer to elute |
| 215 | ESI-MS m/z calc. | ¹H NMR (400 MHz, DMSO-d₆) δ 7.70 (s, | |
| | 547.20, found | 1H), 7.63 (s, 1H), 7.13 (s, 2H), 3.55 (d, J = | |
| | 548.20 (M + 1)⁺; | 13.0 Hz, 1H), 3.41-3.28 (m, 1H), 3.17- | |
| | Retention time: | 3.03 (m, 2H), 2.24-2.13 (m, 2H), 2.13- | |
| | 1.99 minutes (LC | 2.01 (m, 1H), 1.84-1.74 (m, 1H), 1.66- | |
| | Method A) | 1.55 (m, 1H), 1.52-1.41 (m, 1H), 1.01- | |
| | | 0.92 (m, 1H), 0.80-0.66 (m, 1H), 0.59- | |
| | | 0.46 (m, 3H), 0.32-0.14 (m, 4H), | |
| | | 0.13--0.05 (m, 4H). | |
| 216 | ESI-MS m/z calc. | ¹H NMR (400 MHz, DMSO-d₆) δ 7.81 (s, | |
| | 519.17053, found | 1H), 7.74 (s, 1H), 7.24 (s, 2H), 3.71-3.63 | |
| | 520.2 (M + 1)⁺; | (m, 1H), 3.57-3.46 (m, 1H), 3.25-3.12 (m, | |
| | Retention time: | 2H), 2.33-2.13 (m, 3H), 1.93-1.81 (m, | |
| | 1.85 minutes (LC | 1H), 1.73-1.64 (m, 1H), 1.60-1.51 (m, | |
| | Method A) | 1H), 1.49-1.40 (m, 1H), 1.16-1.04 (m, | |
| | | 1H), 1.02-0.95 (m, 1H), 0.92-0.81 (m, | |
| | | 2H), 0.81-0.65 (m, 4H). | |
| 217 | ESI-MS m/z calc. | ¹H NMR (400 MHz, DMSO-d₆) δ 7.78 (s, | |
| | 519.17053, found | 1H), 7.74 (s, 1H), 7.22 (s, 2H), 3.15-3.04 | |
| | 520.2 (M + 1)⁺; | (m, 2H), 2.38-2.30 (m, 1H), 2.26-2.11 (m, | |
| | Retention time: | 2H), 2.06-1.93 (m, 1H), 1.78-1.67 (m, | |
| | 1.77 minutes (LC | 1H), 1.63-1.51 (m, 1H), 1.51-1.42 (m, | |
| | Method A) | 1H), 1.06-0.95 (m, 1H), 0.89-0.78 (m, | |
| | | 1H), 0.76-0.67 (m, 2H), 0.66-0.56 (m, | |
| | | 2H), 0.46-0.33 (m, 2H), 0.27-0.14 (m, 2H). | |
| 218 | ESI-MS m/z calc. | | |
| | 595.1466, found | | |
| | 596.1 (M + 1)⁺; | | |
| | Retention time: | | |
| | 1.78 minutes (LC | | |
| | Method A) | | |
| 219 | ESI-MS m/z calc. | ¹H NMR (400 MHz, methanol-d₄) δ 7.76 (s, | |
| | 587.17676, found | 1H), 7.11 (m, 1H), 7.01-6.92 (m, 2H), 4.96 | |
| | 588.3 (M + 1)⁺; | (d, J = 14.9 Hz, 1H), 4.50 (d, J = 15.0 Hz, | |
| | Retention time: | 1H), 3.22-3.09 (m, 1H), 3.07-2.94 (m, | |
| | 1.57 minutes (LC | 1H), 2.44 (m, 2H), 2.23 (m, 1H), 2.06 (m, | |
| | Method A) | 1H), 2.00-1.88 (m, 1H), 1.86-1.74 (m, | |
| | | 1H), 1.64 (d, 1H), 1.14 (m, 1H), 1.02-0.93 | |
| | | (m, 2H), 0.91-0.78 (m, 1H), 0.76-0.64 (m, | |
| | | 2H). ¹⁹F NMR (376 MHz, methanol-d₄) | |
| | | δ −61.36, −81.17, −124.73 (m, J = 10.7, 3.8 Hz). | |
| 220 | ESI-MS m/z calc. | ¹H NMR (400 MHz, methanol-d₄) δ 7.76 (s, | |
| | 601.1924, found | 1H), 7.33 (dd, J = 7.4, 2.3 Hz, 1H), 7.16 (m, | |
| | 602.3 (M + 1)⁺; | 1H), 6.95 (dd, J = 10.2, 8.3 Hz, 1H), 5.01 (d, | |
| | Retention time: | J = 15.0 Hz, 1H), 4.56 (d, J = 15.0 Hz, 1H), | |
| | 1.67 minutes (LC | 3.72 (p, J = 8.9 Hz, 1H), 3.22-3.16 (m, 1H), | |
| | Method A) | 3.09-2.99 (m, 1H), 2.51-2.40 (m, 2H), | |
| | | 2.38-2.28 (m, 2H), 2.25-2.07 (m, 4H), | |
| | | 1.99-1.92 (m, 1H), 1.89-1.85 (m, 1H), | |
| | | 1.81-1.75 (m, 1H), 1.69-1.62 (m, 1H), | |
| | | 1.18-1.13 (m, 1H), 0.91-0.86 (m, 1H).. | |
| | | ¹⁹F NMR (376 MHz, methanol-d₄) | |
| | | δ −61.33, −79.25-−82.89 (m), −122.25 (m, J = | |
| | | 11.2, 5.8 Hz). | |
| 221 | ESI-MS m/z calc. | ¹H NMR (400 MHz, DMSO-d₆) δ 7.84 (s, | |
| | 581.10645, found | 1H), 7.75 (s, 1H), 7.38-7.28 (m, 3H), 7.23 | |
| | 582.12 (M + 1)⁺; | (s, 1H), 7.12 (d, J = 8.7 Hz, 1H), 4.88 (d, J = | |
| | Retention time: | 15.5 Hz, 1H), 4.55 (d, J = 15.5 Hz, 1H), 3.20 | |
| | 1.92 minutes (LC | (t, J = 12.1 Hz, 1H), 3.07-3.02 (m, 1H), | |
| | Method A) | 2.35-2.25 (m, 2H), 2.24-2.16 (m, 1H), | |
| | | 2.00-1.88 (m, 1H), 1.76-1.65 (m, 1H), | |
| | | 1.60-1.52 (m, 1H), 1.11-1.04 (m, 1H), | |
| | | 0.89-0.81 (m, 1H). | |
| 222 | ESI-MS m/z calc. | ¹H NMR (400 MHz, DMSO-d₆) δ 7.83 (s, | |
| | 615.1328, found | 1H), 7.75 (s, 1H), 7.59 (d, J = 8.7 Hz, 1H), | |
| | 616.19 (M + 1)⁺; | 7.52 (s, 1H), 7.48 (d, J = 9.7 Hz, 1H), 7.32 | |
| | Retention time: | (s, 2H), 4.93 (d, J = 15.7 Hz, 1H), 4.65 (d, | |
| | 1.94 minutes (LC | J = 15.7 Hz, 1H), 3.22 (t, J = 12.8 Hz, 1H), | |
| | Method A) | 3.09-3.02 (m, 1H), 2.40-2.23 (m, 2H), | |
| | | 2.22-2.14 (m, 1H), 1.99-1.88 (m, 1H), | |
| | | 1.74-1.64 (m, 1H), 1.59-1.51 (m, 1H), | |
| | | 1.12-1.04 (m, 1H), 0.90-0.77 (m, 1H). | |
| 223 | ESI-MS m/z calc. | ¹H NMR (400 MHz, methanol-d₄) δ 7.76 (s, | |
| | 587.17676, found | 1H), 7.11-7.01 (m, 2H), 6.97 (dd, J = 9.9, | |
| | 588.3 (M + 1)⁺; | 8.3 Hz, 1H), 4.96 (d, J = 15.4 Hz, 1H), 4.69 | |
| | Retention time: | (d, J = 15.4 Hz, 1H), 3.27-3.19 (m, 1H), | |
| | 1.57 minutes (LC | 3.11-2.98 (m, 1H), 2.45 (m, 2H), 2.24 (m, | |
| | Method A) | 1H), 1.94 (m, 1H), 1.85 (m, 1H), 1.80-1.74 | |
| | | (m, 1H), 1.70-1.61 (m, 1H), 1.17 (m, 1H), | |
| | | 0.91 (m, 2H), 0.88-0.81 (m, 1H), 0.65- | |
| | | 0.55 (m, 2H). ¹⁹F NMR (376 MHz, | |
| | | methanol-d₄) δ −61.37, −81.17, −126.59 | |
| | | (dd, J = 11.0, 5.5 Hz). | |
| 224 | ESI-MS m/z calc. | ¹H NMR (400 MHz, methanol-d₄) δ 7.76 (s, | |
| | 601.1924, found | 1H), 7.26 (dd, J = 7.3, 2.3 Hz, 1H), 7.13 (m, | |
| | 602.3 (M + 1)⁺; | 1H), 7.00 (dd, J = 10.0, 8.4 Hz, 1H), 4.98 (d, | |
| | Retention time: | J = 15.4 Hz, 1H), 4.73 (d, J = 15.4 Hz, 1H), | |
| | 1.68 minutes (LC | 3.56-3.43 (m, 1H), 3.28-3.22 (m, 1H), | |
| | Method A) | 3.11-3.02 (m, 1H), 2.45 (m, 2H), 2.34- | |
| | | 2.26 (m, 2H), 2.26-2.17 (m, 1H), 2.15- | |
| | | 2.03 (m, 2H), 2.02-1.89 (m, 2H), 1.86- | |
| | | 1.74 (m, 2H), 1.72-1.57 (m, 1H), 1.18 (m, | |
| | | 1H), 0.88 (m, 1H); ¹⁹F NMR (376 MHz, | |
| | | methanol-d₄) δ −61.34, −81.19, −126.07 | |
| | | (t, J = 5.7 Hz). | |
| 225 | ESI-MS m/z calc. | ¹H NMR (400 MHz, DMSO-d₆) δ 7.87 (s, | Chiral SFC using a |
| | 587.1967, found | 1H), 7.85 (s, 1H), 7.34 (br. s, 3H), 7.31- | Amylose-1 column and |
| | 588.2 (M + 1)⁺; | 7.22 (m, 2H), 7.10 (d, J = 7.0 Hz, 1H), 4.91 | a gradient of 0% to 25% |
| | Retention time: | (d, J = 15.0 Hz, 1H), 4.48 (d, J = 15.2 Hz, | MeOH and CO₂ over |
| | 3.45 minutes (LC | 1H), 4.02 (d, J = 12.2 Hz, 1H), 3.82 (d, J = | 5 minutes using a flow |
| | Method I) | 12.1 Hz, 1H), 3.61-3.47 (m, 1H), 3.41- | rate of 4 mL/min. First |
| | | 3.33 (m, 1H), 3.27-3.17 (m, 1H), 3.06- | enantiomer to elute |
| | | 2.93 (m, 1H), 2.63-2.53 (m, 1H), 2.46- | |
| | | 2.34 (m, 1H), 1.25 (s, 9H). ¹⁹F NMR (377 | |
| | | MHz, DMSO-d₆) δ −58.80 (s, 3F), −75.56 (s, 3F | |
| 226 | ESI-MS m/z calc. | ¹H NMR (400 MHz, DMSO-d₆) δ 7.87 (s, | Chiral SFC using a |
| | 587.1967, found | 1H), 7.85 (s, 1H), 7.34 (br. s, 3H), 7.31- | Amylose-1 column and |
| | 588.2 (M + 1)⁺; | 7.22 (m, 2H), 7.10 (d, J = 7.1 Hz, 1H), 4.91 | a gradient of 0% to 25% |
| | Retention time: | (d, J = 15.0 Hz, 1H), 4.48 (d, J = 15.2 Hz, | MeOH and CO₂ over |
| | 3.45 minutes (LC | 1H), 4.02 (d, J = 12.3 Hz, 1H), 3.82 (d, J = | 5 minutes using a flow |
| | Method I) | 12.3 Hz, 1H), 3.61-3.47 (m, 1H), 3.39- | rate of 4 mL/min. |
| | | 3.34 (m, 1H), 3.27-3.17 (m, 1H), 3.07- | Second enantiomer to |
| | | 2.91 (m, 1H), 2.65-2.53 (m, 1H), 2.46- | elute |
| | | 2.30 (m, 1H), 1.25 (s, 9H). ¹⁹F NMR (377 | |
| | | MHz, DMSO-d₆) δ −58.80 (s, 3F), −75.57 (s, 3F). | |
| 227 | ESI-MS m/z calc. | ¹H NMR (400 MHz, DMSO-d₆) δ 7.88 (br. s, | Chiral SFC using a |
| | 603.1717, found | 1H), 7.85 (s, 1H), 7.37 (br. s, 2H), 7.06- | Amylose-1 column and |
| | 603.9 (M + 1)⁺; | 6.99 (m, 1H), 6.92 (dd, J = 14.8, 9.9 Hz, 2H), | a gradient of 0% to 30% |
| | Retention time: | 4.88 (d, J = 15.4 Hz, 1H), 4.48 (d, J = 15.4 | MeOH and CO₂ over |
| | 3.57 minutes (LC | Hz, 1H), 4.02 (d, J = 12.2 Hz, 1H), 3.83 (d, | 5 minutes using a flow |
| | Method C) | J = 12.2 Hz, 1H), 3.62-3.43 (m, 2H), 3.41- | rate of 4 mL/min. First |
| | | 3.35 (m, 1H), 3.28-3.18 (m, 1H), 3.08- | enantiomer to elute |
| | | 2.96 (m, 1H), 2.63-2.52 (m, 1H), 2.46- | |
| | | 2.35 (m, 1H), 2.31-2.20 (m, 2H), 2.13- | |
| | | 1.88 (m, 3H), 1.83-1.72 (m, 1H). ¹⁹F NMR | |
| | | (377 MHz, DMSO-d₆) δ −58.83 (s, 3F), −75.58 | |
| | | (s, 3F), −114.15 (t, J = 10.2 Hz, 1F). | |
| 228 | ESI-MS m/z calc. | ¹H NMR (400 MHz, DMSO-d₆) δ 7.87 (s, | Chiral SFC using a |
| | 603.1717, found | 1H), 7.85 (s, 1H), 7.37 (br. s, 2H), 7.05- | Amylose-1 column and |
| | 603.9 (M + 1)⁺; | 6.99 (m, 1H), 6.92 (dd, J = 14.8, 9.9 Hz, 2H), | a gradient of 0% to 30% |
| | Retention time: | 4.88 (d, J = 15.3 Hz, 1H), 4.48 (d, J = 15.4 | MeOH and CO₂ over |
| | 3.52 minutes (LC | Hz, 1H), 4.02 (d, J = 12.3 Hz, 1H), 3.83 (d, | 5 minutes using a flow |
| | Method C) | J = 12.3 Hz, 1H), 3.60-3.43 (m, 2H), 3.41- | rate of 4 mL/min. |
| | | 3.34 (m, 1H), 3.29-3.18 (m, 1H), 3.08- | Second enantiomer to |
| | | 2.95 (m, 1H), 2.64-2.53 (m, 1H), 2.46- | elute |
| | | 2.34 (m, 1H), 2.31-2.20 (m, 2H), 2.13- | |
| | | 2.88 (m, 3H), 1.84-1.72 (m, 1H). ¹⁹F NMR | |
| | | (377 MHz, DMSO-d₆) δ −58.83 (s, 3F), −75.58 | |
| | | (s, 3F), −114.15 (t, J = 10.2 Hz, 1F). | |
| 229 | ESI-MS m/z calc. | ¹H NMR (400 MHz, methanol-d₄) δ 7.76 (s, | |
| | 631.12775, found | 1H), 7.48-7.33 (m, 1H), 7.31 (dd, J = 11.2, | |
| | 632.3 (M + 1)⁺; | 2.1 Hz, 1H), 7.27-7.22 (m, 1H), 5.02 (d, J = | |
| | Retention time: | 15.5 Hz, 1H), 4.61 (d, J = 16.5 Hz, 1H), 4.58 | |
| | 2.05 minutes (LC | (s, 2H), 3.16-3.00 (m, 1H), 2.44 (m, 2H), | |
| | Method A) | 2.24 (m, 1H), 1.97 (dd, J = 10.6, 5.4 Hz, 1H), | |
| | | 1.81 (m, 1H), 1.71-1.56 (m, 1H), 1.17 (m, | |
| | | 1H), 0.89 (m, 1H); ¹⁹F NMR (376 MHz, | |
| | | methanol-d₄) δ −60.65 (d, J = 5.3 | |
| | | Hz), −61.44, −79.94-−81.85 (m), −131.22 (m, | |
| | | J = 17.4, 9.0, 5.2 Hz). | |
| 230 | ESI-MS m/z calc. | ¹H NMR (400 MHz, methanol-d₄) δ 7.76 (s, | |
| | 595.1466, found | 1H), 7.43-7.33 (m, 2H), 7.15-7.09 (m, | |
| | 596.2 (M + 1)⁺; | 2H), 6.79 (t, J = 74.2 Hz, 1H), 5.04 (d, J = | |
| | Retention time: | 15.1 Hz, 1H), 4.56 (d, J = 15.2 Hz, 1H), | |
| | 1.86 minutes (LC | 3.27-3.16 (m, 1H), 3.11-3.00 (m, 1H), 2.51- | |
| | Method A) | 2.38 (m, 2H), 2.29-2.17 (m, 1H), 2.00- | |
| | | 1.88 (m, 1H), 1.83-1.74 (m, 1H), 1.68- | |
| | | 1.61 (m, 1H), 1.20-1.06 (m, 1H), 0.93- | |
| | | 0.85 (m, 1H); ¹⁹F NMR (376 MHz, | |
| | | methanol-d₄) δ −61.44, −81.18, −83.33 (d, | |
| | | J = 74.2 Hz). | |
| 231 | ESI-MS m/z calc. | ¹H NMR (400 MHz, methanol-d₄) δ 7.76 (s, | |
| | 613.13715, found | 1H), 7.32-7.22 (m, 2H), 7.18 (m, 1H), 6.81 | |
| | 614.3 (M + 1)⁺; | (t, J = 73.6 Hz, 1H), 5.02 (d, J = 15.3 Hz, | |
| | Retention time: | 1H), 4.67-4.42 (d, 1H), 3.28-3.17 (m, 1H), | |
| | 1.88 minutes (LC | 3.15-2.95 (m, 1H), 2.44 (m, 2H), 2.33- | |
| | Method A) | 2.16 (m, 1H), 1.97 (m, 1H), 1.80 (m, 1H), | |
| | | 1.70-1.57 (m, 1H), 1.21-1.11 (m, 1H), | |
| | | 0.97-0.85 (m, 1H); ¹⁹F NMR (376 MHz, | |
| | | methanol-d₄) δ −61.43, −81.18, −83.81 (dd, J = | |
| | | 73.6, 3.6 Hz), −132.30 (m, J = 7.7, 3.8 Hz). | |
| 232 | ESI-MS m/z calc. | ¹H NMR (400 MHz, methanol-d₄) δ 8.22 (d, | |
| | 596.14185, found | J = 2.5 Hz, 1H), 7.86 (m, 1H), 7.75 (s, 1H), | |
| | 597.3 (M + 1)⁺; | 7.52 (t, J = 73.1 Hz, 1H), 6.96 (d, J = 8.5 Hz, | |
| | Retention time: | 1H), 5.00 (d, J = 15.1 Hz, 1H), 4.62-4.50 | |
| | 1.8 minutes (LC | (d, 1H), 3.28-3.19 (m, 1H), 3.14-3.02 (m, | |
| | Method A) | 1H), 2.54-2.37 (m, 2H), 2.24 (m, 1H), 2.00 | |
| | | (m, 1H), 1.89-1.75 (m, 1H), 1.72-1.57 (m, | |
| | | 1H), 1.26-1.11 (m, 1H), 0.90 (m, 1H); ¹⁹F | |
| | | NMR (376 MHz, methanol-d₄) | |
| | | δ −61.47, −81.16, −90.23 (d, J = 72.9 Hz). | |
| 233 | ESI-MS m/z calc. | ¹H NMR (400 MHz, methanol-d₄) δ 8.16 (d, | |
| | 596.14185, found | J = 5.2 Hz, 1H), 7.77 (s, 1H), 7.52 (t, J = | |
| | 597.3 (M + 1)⁺; | 73.1 Hz, 1H), 7.15 (dd, J = 5.3, 1.4 Hz, 1H), | |
| | Retention time: | 6.89 (d, J = 1.4 Hz, 1H), 4.98 (d, J = 16.3 | |
| | 1.78 minutes (LC | Hz, 1H), 4.67 (d, J = 16.3 Hz, 1H), 3.44- | |
| | Method A) | 3.35 (m, 1H), 3.19-3.03 (m, 1H), 2.56- | |
| | | 2.38 (m, 2H), 2.29-2.17 (m, 1H), 2.00- | |
| | | 1.87 (m, 1H), 1.85-1.73 (m, 1H), 1.71- | |
| | | 1.56 (m, 1H), 1.22-1.11 (m, 1H), 0.98- | |
| | | 0.80 (m, 1H); ¹⁹F NMR (376 MHz, | |
| | | methanol-d₄) δ −61.44, −81.18, −90.21 | |
| | | (d, J = 73.4 Hz). | |
| 234 | ESI-MS m/z calc. | ¹H NMR (400 MHz, methanol-d₄) δ 8.10- | |
| | 600.19196, found | 7.97 (m, 1H), 7.77 (s, 1H), 6.90 (dd, J = 5.4, | |
| | 601.3 (M + 1)⁺; | 1.4 Hz, 1H), 6.68 (s, 1H), 5.14-5.00 (m, | |
| | Retention time: | 1H), 4.92 (d, J = 16.1 Hz, 1H), 4.63 (d, J = | |
| | 1.66 minutes (LC | 16.1 Hz, 1H), 3.15-3.03 (m, 2H), 2.52- | |
| | Method A) | 2.39 (m, 5H), 2.29-2.20 (m, 1H), 2.16- | |
| | | 2.03 (m, 2H), 2.00-1.89 (m, 1H), 1.81 (dd, | |
| | | J = 10.5, 9.5 Hz, 1H), 1.73-1.59 (m, 2H), | |
| | | 1.23-1.13 (m, 1H), 0.93-0.82 (m, 1H); ¹⁹F | |
| | | NMR (376 MHz, methanol-d₄) δ −61.42, −81.20. | |

### Example 235: Preparation of 3-[[(6R)-17-amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]methyl]benzonitrile (Compound 271)

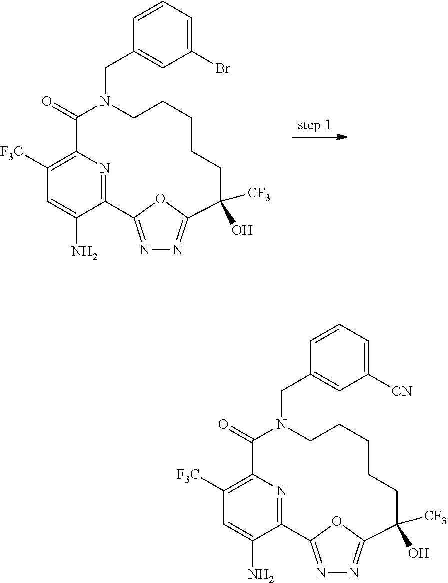 [SMILES: Nc1cc(C(F)(F)F)c2nc1C1=NN=C(O1)[C@@](O)(C(F)(F)F)CCCCCN(Cc1cccc(Br)c1)C2=O ; N#Cc1cccc(CN2CCCCC[C@](O)(C(F)(F)F)C3=NN=C(O3)c3nc(c(C(F)(F)F)cc3N)C2=O)c1]

### Step 1: 3—[[(6R)-17-Amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]methyl]benzonitrile (Compound 271)

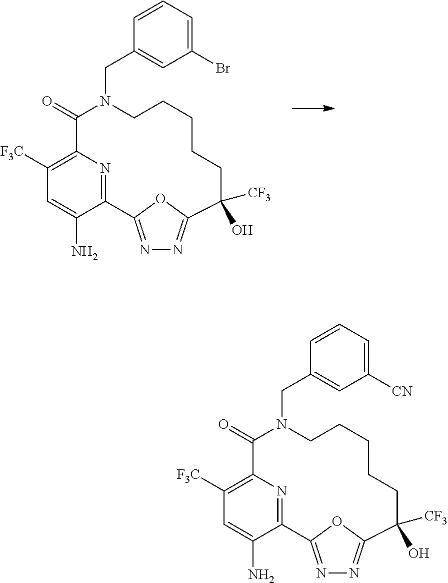 [SMILES: Nc1cc(C(F)(F)F)c2nc1C1=NN=C(O1)[C@@](O)(C(F)(F)F)CCCCCN(Cc1cccc(Br)c1)C2=O ; N#Cc1cccc(CN2CCCCC[C@](O)(C(F)(F)F)C3=NN=C(O3)c3nc(c(C(F)(F)F)cc3N)C2=O)c1]

To (6R)-17-amino-12-[(3-bromophenyl)methyl]-6-hydroxy-6,15-bis(trifluoromethyl)-19-oxa-3,4,12,18-tetrazatricyclo[12.3.1.12,5]nonadeca-1(18),2,4,14,16-pentaen-13-one (12 mg, 0.02 mmol) in DMF (200 μL) was added zinc cyanide (2.7 mg, 0.023 mmol) followed by Pd(PPh₃)₄ (2.7 mg, 0.0023 mmol). The reaction mixture stirred under nitrogen at 90° C. for 5 h, then cooled and diluted with ethyl acetate and partitioned with water. The aqueous layer was extracted with ethyl acetate (2×100 mL) and the combined organic layers were washed with brine, dried over Na₂SO₄, filtered and concentrated. Purification by silica gel chromatography using a gradient from 0% to 30% ethyl acetate/hexanes provided as a white solid, 3-[[(6R)-17-amino-6-hydroxy-13-oxo-6,15-bis(trifluoromethyl)-19-oxa-3,4,12,18-tetrazatricyclo[12.3.1.12,5]nonadeca-1(18),2,4,14,16-pentaen-12-yl]methyl]benzonitrile (5 mg, 41%). ¹H NMR (400 MHz, methanol-d4) δ 7.76 (s, 1H), 7.67 (dd, J=15.8, 8.1 Hz, 3H), 7.53 (t, J=7.7 Hz, 1H), 5.05 (d, J=15.5 Hz, 1H), 4.64 (d, J=15.4 Hz, 1H), 3.26 (d, J=3.6 Hz, 1H), 3.09 (m, 1H), 2.52-2.38 (m, 2H), 2.30-2.17 (m, 1H), 1.97 (d, J=5.5 Hz, 1H), 1.80 (m, 1H), 1.72-1.58 (m, 1H), 1.17 (m, 1H), 0.91 (dd, J=13.9, 9.2 Hz, 1H) ppm. ESI-MS m/z calc. 554.1501, found 555.1 (M+1)⁺; Retention time: 1.64 minutes (LC Method N).

### Example 236: Bioactivity Assay

### Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F; Methods Mol. Biol. 2011:741:39-54). After four days the apical media was removed, and the cells were grown at an air liquid interface for >14 days prior to use. This resulted in a monolayer of fully differentiated columnar cells that were ciliated, which are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl₂, 1.2 CaCl₂), 10 Glucose, 10 HEPES (pH adjusted to 7.4 with NaOH) and the apical solution contained (in mM) 145 NaGluconate, 1.2 MgCl₂, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101 and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

### Identification of Potentiator Compounds

The activity of the CFTR potentiator compounds on the CFTR-mediated I_{SC} was determined in Ussing chamber studies as described above. The F508del/MF-HBE cell cultures were incubated with the potentiator compounds at a range of concentrations in combination with 10 μM (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione for 18-24 hours at 37° C. and in the presence of 20% human serum. The concentration of potentiator compounds and (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione used during the 18-24 hours incubations was kept constant throughout the Ussing chamber measurement of the CFTR-mediated I_{SC} to ensure compounds were present throughout the entire experiment. The efficacy and potency of the putative F508del potentiators was compared to that of the known potentiator, ivacaftor (N-[2,4-bis(1,1-dimethylethyl)-5-hydroxyphenyl]-1,4-dihydro-4-oxoquinoline-3-carboxamide).

The following table represents CFTR modulating activity for representative compounds of the invention generated using the assay described in this example (EC₅₀: +++ is <500 nM; ++ is 500 nM−1 μM; + is >1 μM; and ND is “not determined in this assay”).

TABLE 7
Bioactivity

| Com- | | |
|---|---|---|
| pound | | |
| No. | Structure | EC₅₀ |
| 1 | 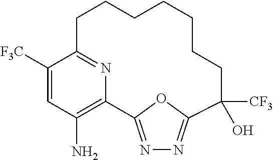 [SMILES: Nc1cc(C(F)(F)F)c2nc1-c1nnc(o1)C(O)(C(F)(F)F)CCCCCCC2] | ND |
| 2 | 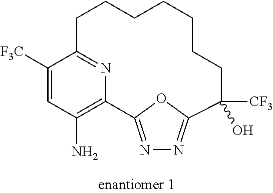 [SMILES: Nc1cc(C(F)(F)F)c2nc1-c1nnc(o1)C(O)(C(F)(F)F)CCCCCCC2] | +++ |
| 3 | 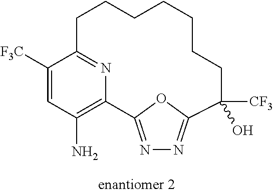 [SMILES: Nc1cc(C(F)(F)F)c2nc1-c1nnc(o1)C(O)(C(F)(F)F)CCCCCCC2] | ND |
| 4 | 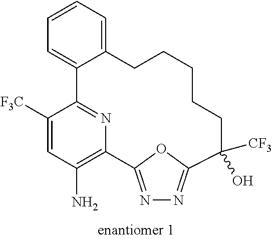 [SMILES: Nc1cc(C(F)(F)F)c2nc1-c1nnc(o1)C(O)(C(F)(F)F)CCCCCc1ccccc1-2] | +++ |
| 5 | 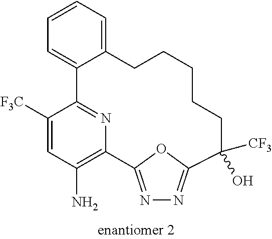 [SMILES: Nc1cc(C(F)(F)F)c2nc1-c1nnc(o1)C(O)(C(F)(F)F)CCCCCc1ccccc1-2] | + |
| 6 | 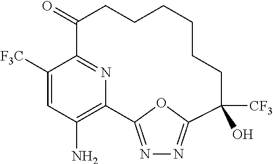 [SMILES: Nc1cc(C(F)(F)F)c2nc1-c1nnc(o1)[C@@](O)(C(F)(F)F)CCCCCCC2=O] | +++ |
| 7 | 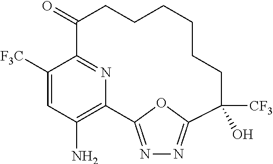 [SMILES: Nc1cc(C(F)(F)F)c2nc1-c1nnc(o1)[C@](O)(C(F)(F)F)CCCCCCC2=O] | + |
| 8 | 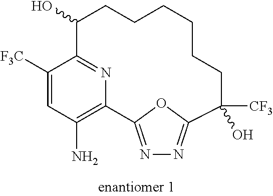 [SMILES: Nc1cc(C(F)(F)F)c2nc1-c1nnc(o1)C(O)(C(F)(F)F)CCCCCCC2O] | +++ |
| 9 | 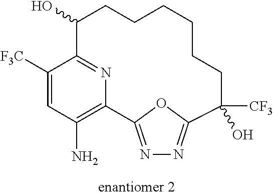 [SMILES: Nc1cc(C(F)(F)F)c2nc1-c1nnc(o1)C(O)(C(F)(F)F)CCCCCCC2O] | + |
| 10 | 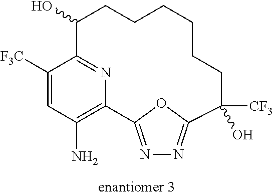 [SMILES: Nc1cc(C(F)(F)F)c2nc1-c1nnc(o1)C(O)(C(F)(F)F)CCCCCCC2O] | +++ |
| 11 | 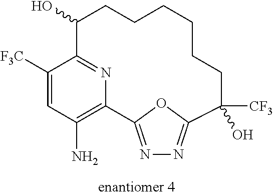 [SMILES: Nc1cc(C(F)(F)F)c2nc1-c1nnc(o1)C(O)(C(F)(F)F)CCCCCCC2O] | + |
| 12 | 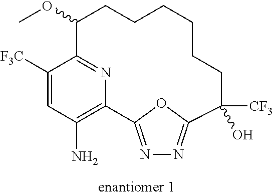 [SMILES: COC1CCCCCCC(O)(C(F)(F)F)c2nnc(o2)-c2nc1c(C(F)(F)F)cc2N] | +++ |
| 13 | 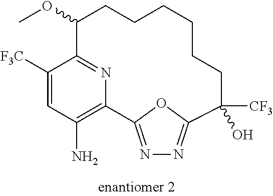 [SMILES: COC1CCCCCCC(O)(C(F)(F)F)c2nnc(o2)-c2nc1c(C(F)(F)F)cc2N] | +++ |
| 14 | 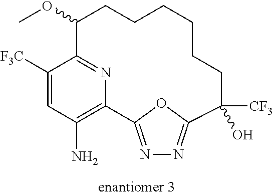 [SMILES: COC1CCCCCCC(O)(C(F)(F)F)c2nnc(o2)-c2nc1c(C(F)(F)F)cc2N] | +++ |
| 15 | 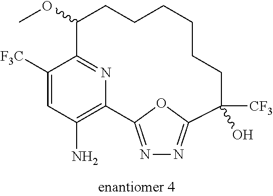 [SMILES: COC1CCCCCCC(O)(C(F)(F)F)c2nnc(o2)-c2nc1c(C(F)(F)F)cc2N] | ++ |
| 16 | 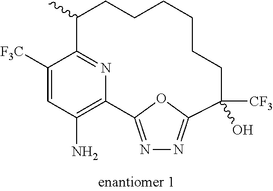 [SMILES: CC1CCCCCCC(O)(C(F)(F)F)c2nnc(o2)-c2nc1c(C(F)(F)F)cc2N] | +++ |
| 17 | 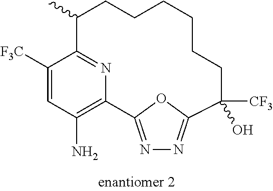 [SMILES: CC1CCCCCCC(O)(C(F)(F)F)c2nnc(o2)-c2nc1c(C(F)(F)F)cc2N] | +++ |
| 18 | 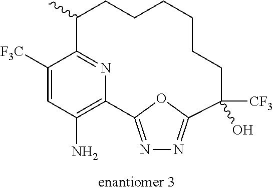 [SMILES: CC1CCCCCCC(O)(C(F)(F)F)c2nnc(o2)-c2nc1c(C(F)(F)F)cc2N] | +++ |
| 19 | 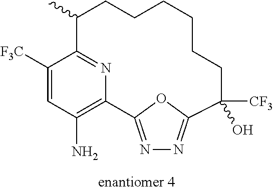 [SMILES: CC1CCCCCCC(O)(C(F)(F)F)c2nnc(o2)-c2nc1c(C(F)(F)F)cc2N] | +++ |
| 20 | 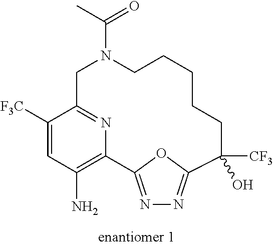 [SMILES: CC(=O)N1CCCCCC(O)(C(F)(F)F)c2nnc(o2)-c2nc(c(C(F)(F)F)cc2N)C1] | +++ |
| 21 | 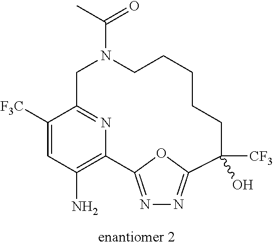 [SMILES: CC(=O)N1CCCCCC(O)(C(F)(F)F)c2nnc(o2)-c2nc(c(C(F)(F)F)cc2N)C1] | + |
| 22 | 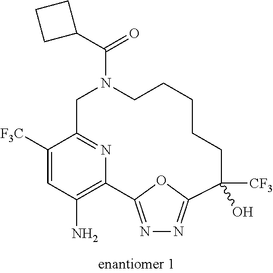 [SMILES: Nc1cc(C(F)(F)F)c2nc1-c1nnc(o1)C(O)(C(F)(F)F)CCCCCN(C(=O)C1CCC1)C2] | +++ |
| 23 | 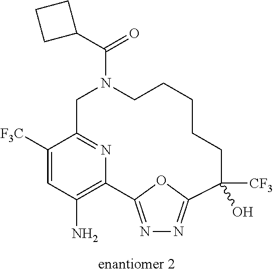 [SMILES: Nc1cc(C(F)(F)F)c2nc1-c1nnc(o1)C(O)(C(F)(F)F)CCCCCN(C(=O)C1CCC1)C2] | + |
| 24 | 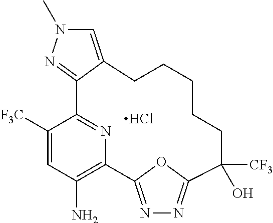 [SMILES: Cn1cc2c(n1)-c1nc(c(N)cc1C(F)(F)F)-c1nnc(o1)C(O)(C(F)(F)F)CCCCC2 ; Cl] | + |
| 25 | 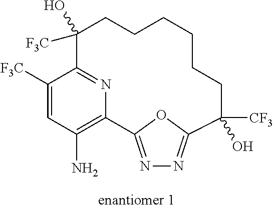 [SMILES: Nc1cc(C(F)(F)F)c2nc1-c1nnc(o1)C(O)(C(F)(F)F)CCCCCCC2(O)C(F)(F)F] | + |
| 26 | 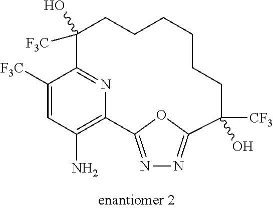 [SMILES: Nc1cc(C(F)(F)F)c2nc1-c1nnc(o1)C(O)(C(F)(F)F)CCCCCCC2(O)C(F)(F)F] | +++ |
| 27 | 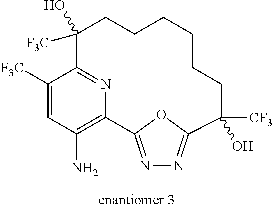 [SMILES: Nc1cc(C(F)(F)F)c2nc1-c1nnc(o1)C(O)(C(F)(F)F)CCCCCCC2(O)C(F)(F)F] | +++ |
| 28 | 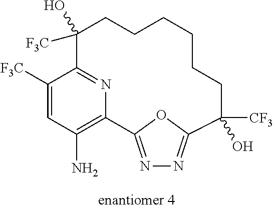 [SMILES: Nc1cc(C(F)(F)F)c2nc1-c1nnc(o1)C(O)(C(F)(F)F)CCCCCCC2(O)C(F)(F)F] | + |
| 29 | 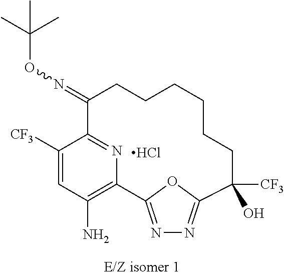 [SMILES: CC(C)(C)ON=C1CCCCCC[C@](O)(C(F)(F)F)c2nnc(o2)-c2nc1c(C(F)(F)F)cc2N ; Cl] | +++ |
| 30 | 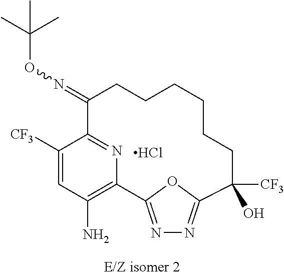 [SMILES: CC(C)(C)ON=C1CCCCCC[C@](O)(C(F)(F)F)c2nnc(o2)-c2nc1c(C(F)(F)F)cc2N ; Cl] | +++ |
| 31 | 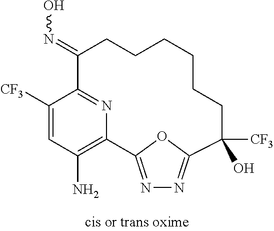 [SMILES: Nc1cc(C(F)(F)F)c2nc1-c1nnc(o1)[C@@](O)(C(F)(F)F)CCCCCCC2=NO] | +++ |
| 32 | 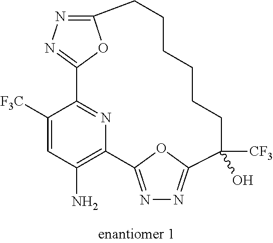 [SMILES: Nc1cc(C(F)(F)F)c2nc1-c1nnc(o1)C(O)(C(F)(F)F)CCCCCCC1=NN=C2O1] | +++ |
| 33 | 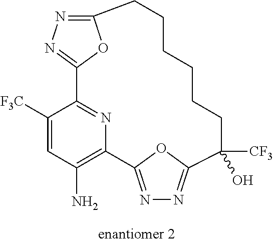 [SMILES: Nc1cc(C(F)(F)F)c2nc1-c1nnc(o1)C(O)(C(F)(F)F)CCCCCCC1=NN=C2O1] | + |
| 34 | 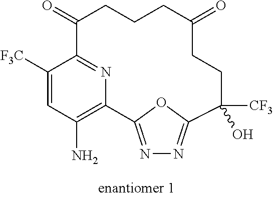 [SMILES: Nc1cc(C(F)(F)F)c2nc1-c1nnc(o1)C(O)(C(F)(F)F)CCC(=O)CCCC2=O] | +++ |
| 35 | 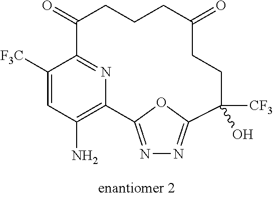 [SMILES: Nc1cc(C(F)(F)F)c2nc1-c1nnc(o1)C(O)(C(F)(F)F)CCC(=O)CCCC2=O] | + |
| 36 | 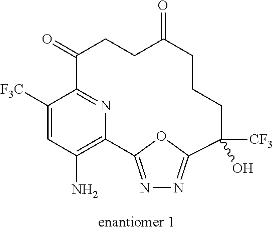 [SMILES: Nc1cc(C(F)(F)F)c2nc1-c1nnc(o1)C(O)(C(F)(F)F)CCCC(=O)CCC2=O] | + |
| 37 | 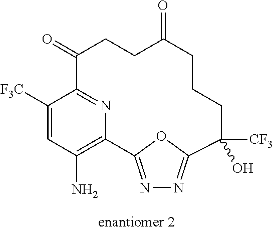 [SMILES: Nc1cc(C(F)(F)F)c2nc1-c1nnc(o1)C(O)(C(F)(F)F)CCCC(=O)CCC2=O] | + |
| 38 | 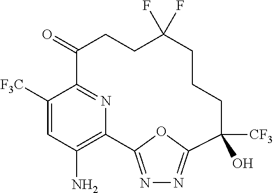 [SMILES: Nc1cc(C(F)(F)F)c2nc1-c1nnc(o1)[C@@](O)(C(F)(F)F)CCCC(F)(F)CCC2=O] | +++ |
| 39 | 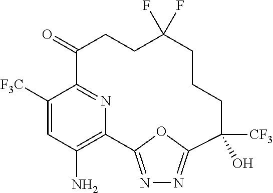 [SMILES: Nc1cc(C(F)(F)F)c2nc1-c1nnc(o1)[C@](O)(C(F)(F)F)CCCC(F)(F)CCC2=O] | ++ |
| 40 | 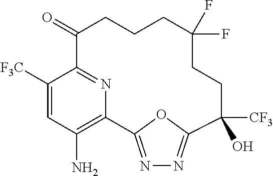 [SMILES: Nc1cc(C(F)(F)F)c2nc1-c1nnc(o1)[C@@](O)(C(F)(F)F)CCC(F)(F)CCCC2=O] | +++ |
| 41 | 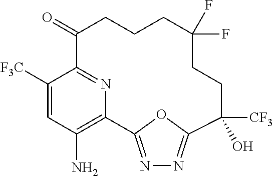 [SMILES: Nc1cc(C(F)(F)F)c2nc1-c1nnc(o1)[C@](O)(C(F)(F)F)CCC(F)(F)CCCC2=O] | +++ |
| 42 | 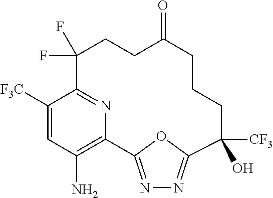 [SMILES: Nc1cc(C(F)(F)F)c2nc1-c1nnc(o1)[C@@](O)(C(F)(F)F)CCCC(=O)CCC2(F)F] | +++ |
| 43 | 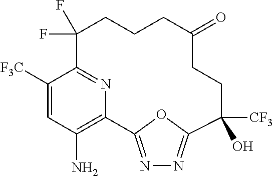 [SMILES: Nc1cc(C(F)(F)F)c2nc1-c1nnc(o1)[C@@](O)(C(F)(F)F)CCC(=O)CCCC2(F)F] | +++ |
| 44 | 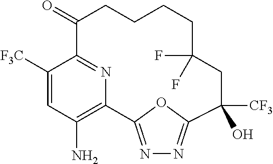 [SMILES: Nc1cc(C(F)(F)F)c2nc1-c1nnc(o1)[C@@](O)(C(F)(F)F)CC(F)(F)CCCCC2=O] | +++ |
| 45 | 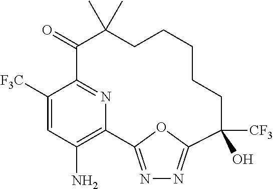 [SMILES: CC1(C)CCCCC[C@](O)(C(F)(F)F)c2nnc(o2)-c2nc(c(C(F)(F)F)cc2N)C1=O] | +++ |
| 46 | 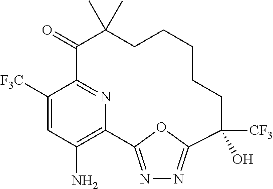 [SMILES: CC1(C)CCCCC[C@@](O)(C(F)(F)F)c2nnc(o2)-c2nc(c(C(F)(F)F)cc2N)C1=O] | +++ |
| 47 | 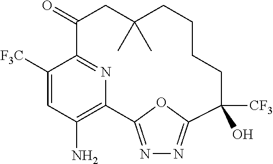 [SMILES: CC1(C)CCCC[C@](O)(C(F)(F)F)c2nnc(o2)-c2nc(c(C(F)(F)F)cc2N)C(=O)C1] | +++ |
| 48 | 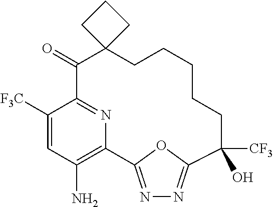 [SMILES: Nc1cc(C(F)(F)F)c2nc1-c1nnc(o1)[C@@](O)(C(F)(F)F)CCCCCC1(CCC1)C2=O] | +++ |
| 49 | 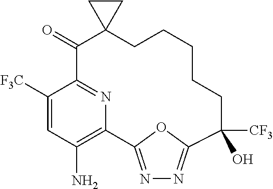 [SMILES: Nc1cc(C(F)(F)F)c2nc1-c1nnc(o1)[C@@](O)(C(F)(F)F)CCCCCC1(CC1)C2=O] | +++ |
| 50 | 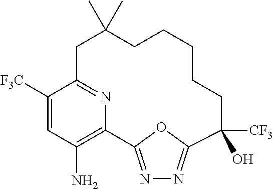 [SMILES: CC1(C)CCCCC[C@](O)(C(F)(F)F)c2nnc(o2)-c2nc(c(C(F)(F)F)cc2N)C1] | +++ |
| 51 | 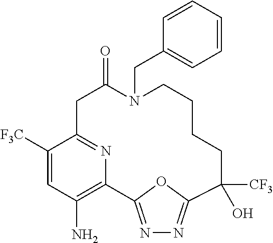 [SMILES: Nc1cc(C(F)(F)F)c2nc1-c1nnc(o1)C(O)(C(F)(F)F)CCCCN(Cc1ccccc1)C(=O)C2] | +++ |
| 52 | 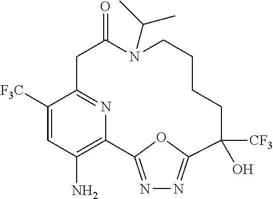 [SMILES: CC(C)N1CCCCC(O)(C(F)(F)F)c2nnc(o2)-c2nc(c(C(F)(F)F)cc2N)CC1=O] | + |
| 53 | 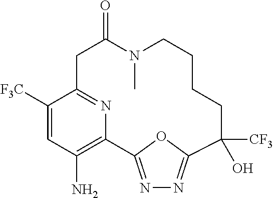 [SMILES: CN1CCCCC(O)(C(F)(F)F)c2nnc(o2)-c2nc(c(C(F)(F)F)cc2N)CC1=O] | + |
| 54 | 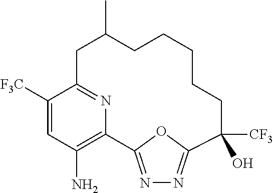 [SMILES: CC1CCCCC[C@](O)(C(F)(F)F)c2nnc(o2)-c2nc(c(C(F)(F)F)cc2N)C1] | ND |
| 55 | 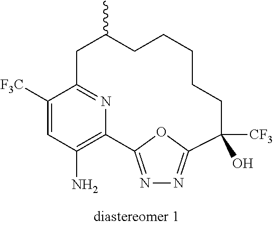 [SMILES: CC1CCCCC[C@](O)(C(F)(F)F)c2nnc(o2)-c2nc(c(C(F)(F)F)cc2N)C1] | +++ |
| 56 | 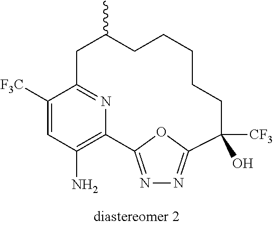 [SMILES: CC1CCCCC[C@](O)(C(F)(F)F)c2nnc(o2)-c2nc(c(C(F)(F)F)cc2N)C1] | +++ |
| 57 | 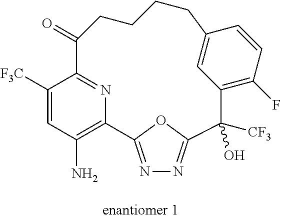 [SMILES: Nc1cc(C(F)(F)F)c2nc1-c1nnc(o1)C(O)(C(F)(F)F)c1cc(ccc1F)CCCCC2=O] | +++ |
| 58 | 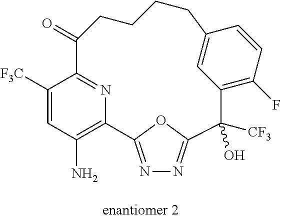 [SMILES: Nc1cc(C(F)(F)F)c2nc1-c1nnc(o1)C(O)(C(F)(F)F)c1cc(ccc1F)CCCCC2=O] | ++ |
| 59 | 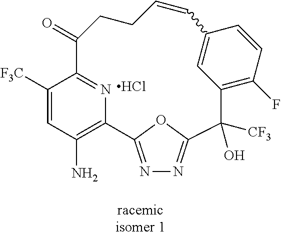 [SMILES: Nc1cc(C(F)(F)F)c2nc1-c1nnc(o1)C(O)(C(F)(F)F)c1cc(ccc1F)C=CCCC2=O ; Cl] | + |
| 60 | 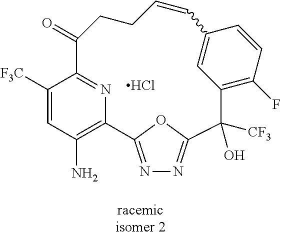 [SMILES: Nc1cc(C(F)(F)F)c2nc1-c1nnc(o1)C(O)(C(F)(F)F)c1cc(ccc1F)C=CCCC2=O ; Cl] | +++ |
| 61 | 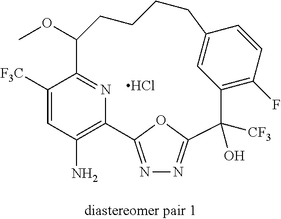 [SMILES: COC1CCCCc2ccc(F)c(c2)C(O)(C(F)(F)F)c2nnc(o2)-c2nc1c(C(F)(F)F)cc2N ; Cl] | +++ |
| 62 | 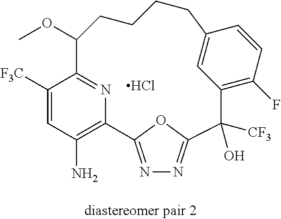 [SMILES: COC1CCCCc2ccc(F)c(c2)C(O)(C(F)(F)F)c2nnc(o2)-c2nc1c(C(F)(F)F)cc2N ; Cl] | +++ |
| 63 | 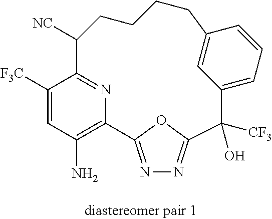 [SMILES: N#CC1CCCCc2cccc(c2)C(O)(C(F)(F)F)c2nnc(o2)-c2nc1c(C(F)(F)F)cc2N] | +++ |
| 64 | 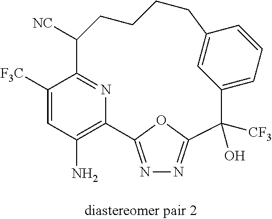 [SMILES: N#CC1CCCCc2cccc(c2)C(O)(C(F)(F)F)c2nnc(o2)-c2nc1c(C(F)(F)F)cc2N] | +++ |
| 65 | 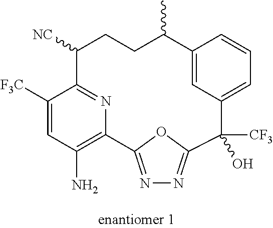 [SMILES: CC1CCC(C#N)c2nc(c(N)cc2C(F)(F)F)-c2nnc(o2)C(O)(C(F)(F)F)c2cccc1c2] | ND |
| 66 | 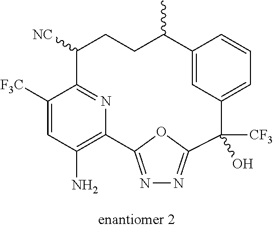 [SMILES: CC1CCC(C#N)c2nc(c(N)cc2C(F)(F)F)-c2nnc(o2)C(O)(C(F)(F)F)c2cccc1c2] | ND |
| 67 | 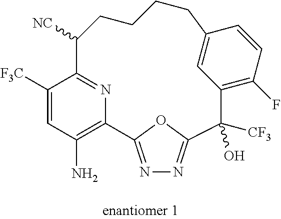 [SMILES: N#CC1CCCCc2ccc(F)c(c2)C(O)(C(F)(F)F)c2nnc(o2)-c2nc1c(C(F)(F)F)cc2N] | +++ |
| 68 | 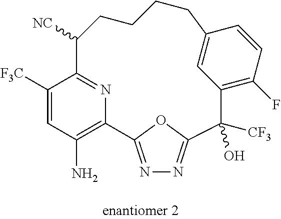 [SMILES: N#CC1CCCCc2ccc(F)c(c2)C(O)(C(F)(F)F)c2nnc(o2)-c2nc1c(C(F)(F)F)cc2N] | +++ |
| 69 | 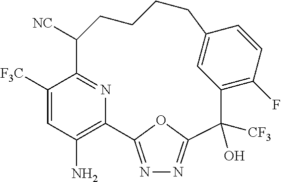 [SMILES: N#CC1CCCCc2ccc(F)c(c2)C(O)(C(F)(F)F)c2nnc(o2)-c2nc1c(C(F)(F)F)cc2N] | +++ |
| 70 | 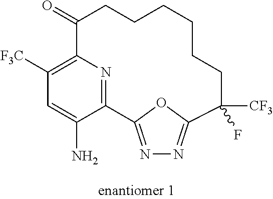 [SMILES: Nc1cc(C(F)(F)F)c2nc1-c1nnc(o1)C(F)(C(F)(F)F)CCCCCCC2=O] | ND |
| 71 | 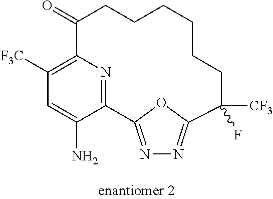 [SMILES: Nc1cc(C(F)(F)F)c2nc1-c1nnc(o1)C(F)(C(F)(F)F)CCCCCCC2=O] | + |
| 72 | 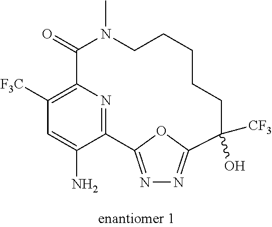 [SMILES: CN1CCCCCC(O)(C(F)(F)F)c2nnc(o2)-c2nc(c(C(F)(F)F)cc2N)C1=O] | +++ |
| 73 | 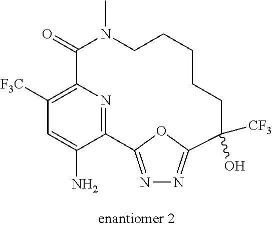 [SMILES: CN1CCCCCC(O)(C(F)(F)F)c2nnc(o2)-c2nc(c(C(F)(F)F)cc2N)C1=O] | + |
| 74 | 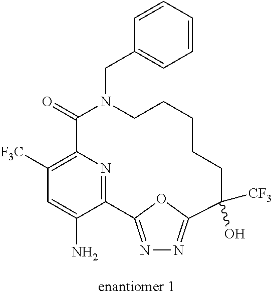 [SMILES: Nc1cc(C(F)(F)F)c2nc1-c1nnc(o1)C(O)(C(F)(F)F)CCCCCN(Cc1ccccc1)C2=O] | +++ |
| 75 | 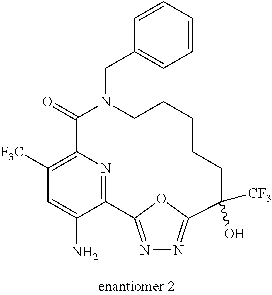 [SMILES: Nc1cc(C(F)(F)F)c2nc1-c1nnc(o1)C(O)(C(F)(F)F)CCCCCN(Cc1ccccc1)C2=O] | ND |
| 76 | 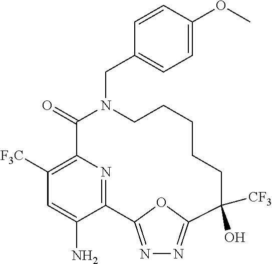 [SMILES: COc1ccc(CN2CCCCC[C@](O)(C(F)(F)F)c3nnc(o3)-c3nc(c(C(F)(F)F)cc3N)C2=O)cc1] | +++ |
| 77 | 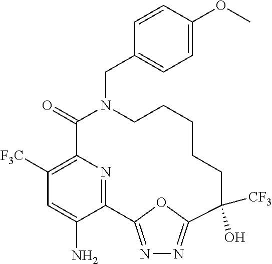 [SMILES: COc1ccc(CN2CCCCC[C@@](O)(C(F)(F)F)c3nnc(o3)-c3nc(c(C(F)(F)F)cc3N)C2=O)cc1] | ND |
| 78 | 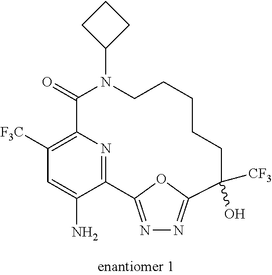 [SMILES: Nc1cc(C(F)(F)F)c2nc1-c1nnc(o1)C(O)(C(F)(F)F)CCCCCN(C1CCC1)C2=O] | +++ |
| 79 | 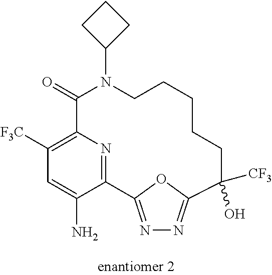 [SMILES: Nc1cc(C(F)(F)F)c2nc1-c1nnc(o1)C(O)(C(F)(F)F)CCCCCN(C1CCC1)C2=O] | ND |
| 80 | 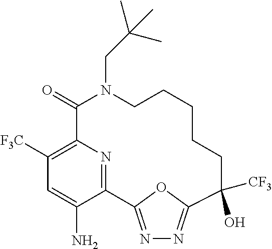 [SMILES: CC(C)(C)CN1CCCCC[C@](O)(C(F)(F)F)c2nnc(o2)-c2nc(c(C(F)(F)F)cc2N)C1=O] | +++ |
| 81 | 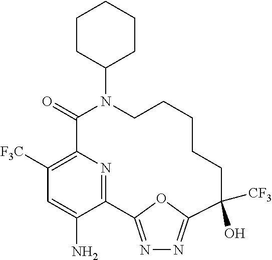 [SMILES: Nc1cc(C(F)(F)F)c2nc1-c1nnc(o1)[C@@](O)(C(F)(F)F)CCCCCN(C1CCCCC1)C2=O] | +++ |
| 82 | 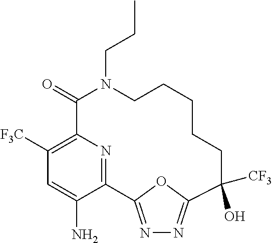 [SMILES: CCCN1CCCCC[C@](O)(C(F)(F)F)c2nnc(o2)-c2nc(c(C(F)(F)F)cc2N)C1=O] | +++ |
| 83 | 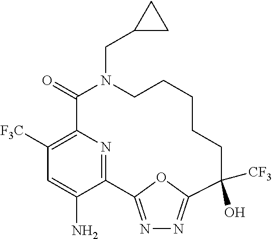 [SMILES: Nc1cc(C(F)(F)F)c2nc1-c1nnc(o1)[C@@](O)(C(F)(F)F)CCCCCN(CC1CC1)C2=O] | +++ |
| 84 | 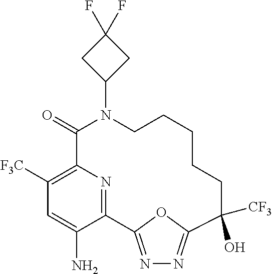 [SMILES: Nc1cc(C(F)(F)F)c2nc1-c1nnc(o1)[C@@](O)(C(F)(F)F)CCCCCN(C1CC(F)(F)C1)C2=O] | +++ |
| 85 | 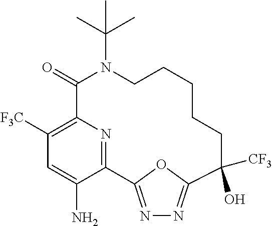 [SMILES: CC(C)(C)N1CCCCC[C@](O)(C(F)(F)F)c2nnc(o2)-c2nc(c(C(F)(F)F)cc2N)C1=O] | +++ |
| 86 | 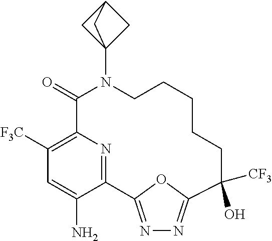 [SMILES: Nc1cc(C(F)(F)F)c2nc1-c1nnc(o1)[C@@](O)(C(F)(F)F)CCCCCN(C13CC(C1)C3)C2=O] | +++ |
| 87 | 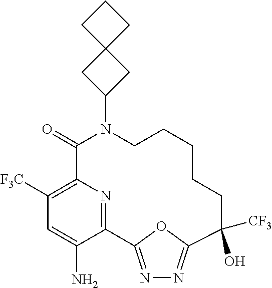 [SMILES: Nc1cc(C(F)(F)F)c2nc1-c1nnc(o1)[C@@](O)(C(F)(F)F)CCCCCN(C1CC3(CCC3)C1)C2=O] | +++ |
| 88 | 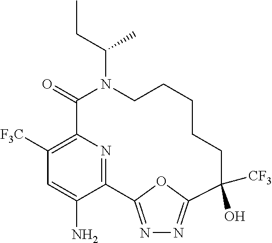 [SMILES: CC[C@H](C)N1CCCCC[C@](O)(C(F)(F)F)c2nnc(o2)-c2nc(c(C(F)(F)F)cc2N)C1=O] | +++ |
| 89 | 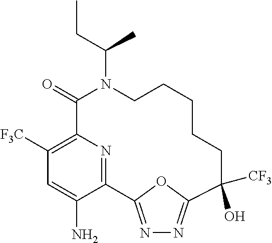 [SMILES: CC[C@@H](C)N1CCCCC[C@](O)(C(F)(F)F)c2nnc(o2)-c2nc(c(C(F)(F)F)cc2N)C1=O] | +++ |
| 90 | 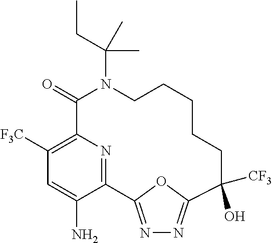 [SMILES: CCC(C)(C)N1CCCCC[C@](O)(C(F)(F)F)c2nnc(o2)-c2nc(c(C(F)(F)F)cc2N)C1=O] | +++ |
| 91 | 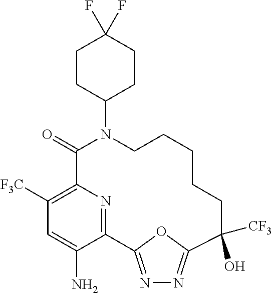 [SMILES: Nc1cc(C(F)(F)F)c2nc1-c1nnc(o1)[C@@](O)(C(F)(F)F)CCCCCN(C1CCC(F)(F)CC1)C2=O] | +++ |
| 92 | 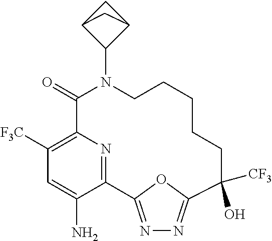 [SMILES: Nc1cc(C(F)(F)F)c2nc1-c1nnc(o1)[C@@](O)(C(F)(F)F)CCCCCN(C1C3CC1C3)C2=O] | +++ |
| 93 | 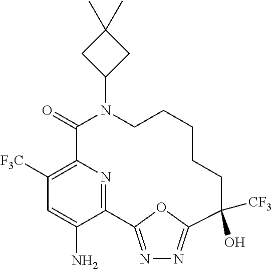 [SMILES: CC1(C)CC(N2CCCCC[C@](O)(C(F)(F)F)c3nnc(o3)-c3nc(c(C(F)(F)F)cc3N)C2=O)C1] | +++ |
| 94 | 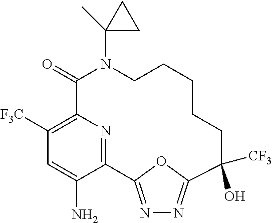 [SMILES: CC1(N2CCCCC[C@](O)(C(F)(F)F)c3nnc(o3)-c3nc(c(C(F)(F)F)cc3N)C2=O)CC1] | +++ |
| 95 | 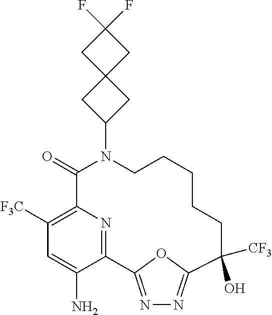 [SMILES: Nc1cc(C(F)(F)F)c2nc1-c1nnc(o1)[C@@](O)(C(F)(F)F)CCCCCN(C1CC3(C1)CC(F)(F)C3)C2=O] | +++ |
| 96 | 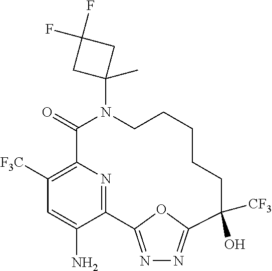 [SMILES: CC1(N2CCCCC[C@](O)(C(F)(F)F)c3nnc(o3)-c3nc(c(C(F)(F)F)cc3N)C2=O)CC(F)(F)C1] | +++ |
| 97 | 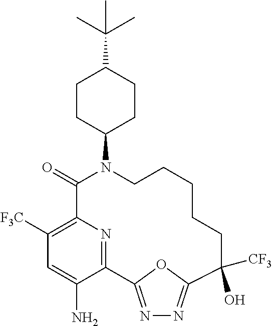 | +++ |
| 98 | 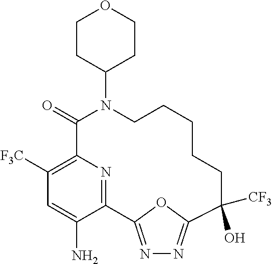 [SMILES: Nc1cc(C(F)(F)F)c2nc1-c1nnc(o1)[C@@](O)(C(F)(F)F)CCCCCN(C1CCOCC1)C2=O] | +++ |
| 99 | 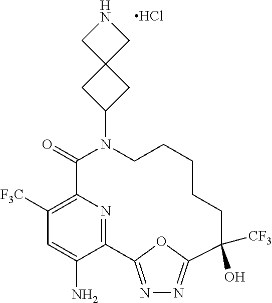 [SMILES: Cl ; Nc1cc(C(F)(F)F)c2nc1-c1nnc(o1)[C@@](O)(C(F)(F)F)CCCCCN(C1CC3(CNC3)C1)C2=O] | + |
| 100 | 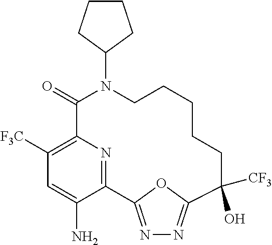 [SMILES: Nc1cc(C(F)(F)F)c2nc1-c1nnc(o1)[C@@](O)(C(F)(F)F)CCCCCN(C1CCCC1)C2=O] | +++ |
| 101 | 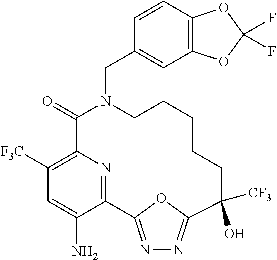 [SMILES: Nc1cc(C(F)(F)F)c2nc1-c1nnc(o1)[C@@](O)(C(F)(F)F)CCCCCN(Cc1ccc3c(c1)OC(F)(F)O3)C2=O] | +++ |
| 102 | 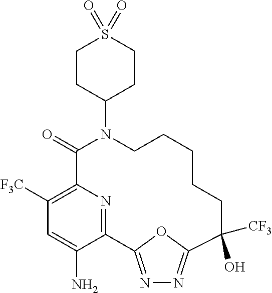 [SMILES: Nc1cc(C(F)(F)F)c2nc1-c1nnc(o1)[C@@](O)(C(F)(F)F)CCCCCN(C1CCS(=O)(=O)CC1)C2=O] | + |
| 103 | 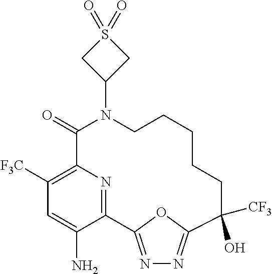 [SMILES: Nc1cc(C(F)(F)F)c2nc1-c1nnc(o1)[C@@](O)(C(F)(F)F)CCCCCN(C1CS(=O)(=O)C1)C2=O] | + |
| 104 | 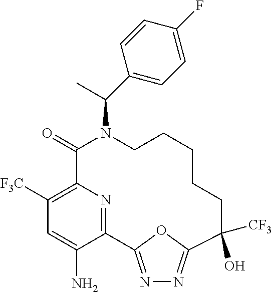 [SMILES: C[C@@H](c1ccc(F)cc1)N1CCCCC[C@](O)(C(F)(F)F)c2nnc(o2)-c2nc(c(C(F)(F)F)cc2N)C1=O] | +++ |
| 105 | 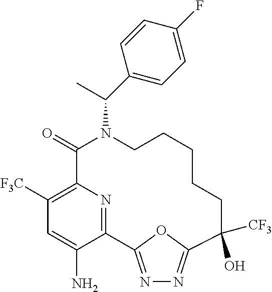 [SMILES: C[C@H](c1ccc(F)cc1)N1CCCCC[C@](O)(C(F)(F)F)c2nnc(o2)-c2nc(c(C(F)(F)F)cc2N)C1=O] | +++ |
| 106 | 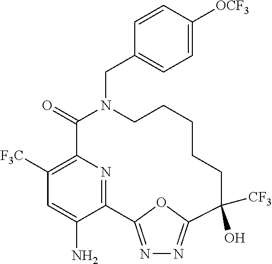 [SMILES: Nc1cc(C(F)(F)F)c2nc1-c1nnc(o1)[C@@](O)(C(F)(F)F)CCCCCN(Cc1ccc(OC(F)(F)F)cc1)C2=O] | +++ |
| 107 | 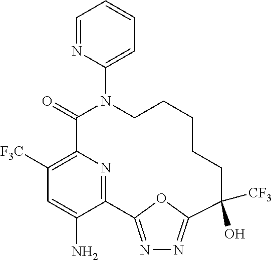 [SMILES: Nc1cc(C(F)(F)F)c2nc1-c1nnc(o1)[C@@](O)(C(F)(F)F)CCCCCN(c1ccccn1)C2=O] | +++ |
| 108 | 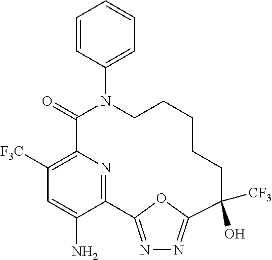 [SMILES: Nc1cc(C(F)(F)F)c2nc1-c1nnc(o1)[C@@](O)(C(F)(F)F)CCCCCN(c1ccccc1)C2=O] | +++ |
| 109 | 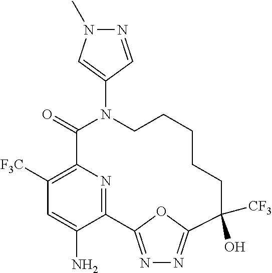 [SMILES: Cn1cc(N2CCCCC[C@](O)(C(F)(F)F)c3nnc(o3)-c3nc(c(C(F)(F)F)cc3N)C2=O)cn1] | +++ |
| 110 | 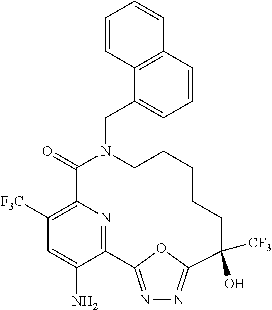 [SMILES: Nc1cc(C(F)(F)F)c2nc1-c1nnc(o1)[C@@](O)(C(F)(F)F)CCCCCN(Cc1cccc3ccccc13)C2=O] | +++ |
| 111 | 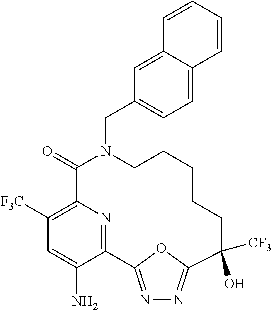 [SMILES: Nc1cc(C(F)(F)F)c2nc1-c1nnc(o1)[C@@](O)(C(F)(F)F)CCCCCN(Cc1ccc3ccccc3c1)C2=O] | +++ |
| 112 | 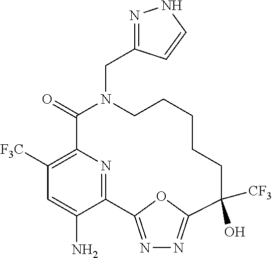 [SMILES: Nc1cc(C(F)(F)F)c2nc1-c1nnc(o1)[C@@](O)(C(F)(F)F)CCCCCN(Cc1cc[nH]n1)C2=O] | + |
| 113 | 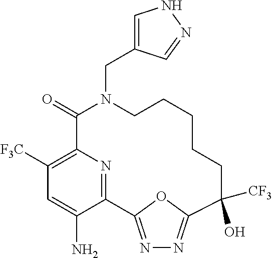 [SMILES: Nc1cc(C(F)(F)F)c2nc1-c1nnc(o1)[C@@](O)(C(F)(F)F)CCCCCN(Cc1cn[nH]c1)C2=O] | + |
| 114 | 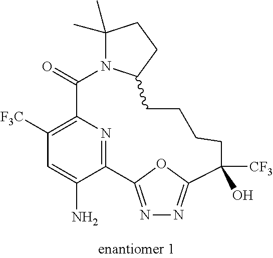 [SMILES: CC1(C)CCC2CCCC[C@](O)(C(F)(F)F)c3nnc(o3)-c3nc(c(C(F)(F)F)cc3N)C(=O)N21] | +++ |
| 115 | 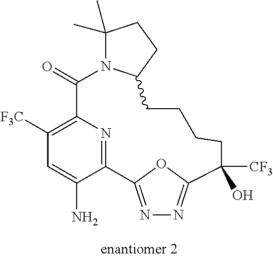 [SMILES: CC1(C)CCC2CCCC[C@](O)(C(F)(F)F)c3nnc(o3)-c3nc(c(C(F)(F)F)cc3N)C(=O)N21] | +++ |
| 116 | 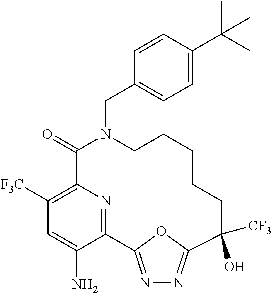 [SMILES: CC(C)(C)c1ccc(CN2CCCCC[C@](O)(C(F)(F)F)c3nnc(o3)-c3nc(c(C(F)(F)F)cc3N)C2=O)cc1] | +++ |
| 117 | 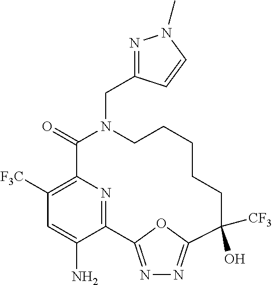 [SMILES: Cn1ccc(CN2CCCCC[C@](O)(C(F)(F)F)c3nnc(o3)-c3nc(c(C(F)(F)F)cc3N)C2=O)n1] | + |
| 118 | 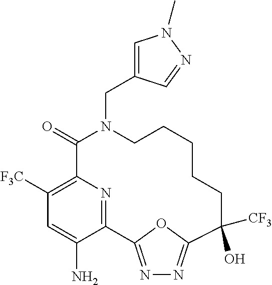 [SMILES: Cn1cc(CN2CCCCC[C@](O)(C(F)(F)F)c3nnc(o3)-c3nc(c(C(F)(F)F)cc3N)C2=O)cn1] | + |
| 119 | 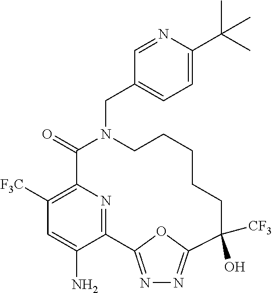 [SMILES: CC(C)(C)c1ccc(CN2CCCCC[C@](O)(C(F)(F)F)c3nnc(o3)-c3nc(c(C(F)(F)F)cc3N)C2=O)cn1] | +++ |
| 120 | 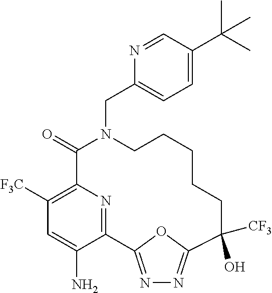 [SMILES: CC(C)(C)c1ccc(CN2CCCCC[C@](O)(C(F)(F)F)c3nnc(o3)-c3nc(c(C(F)(F)F)cc3N)C2=O)nc1] | +++ |
| 121 | 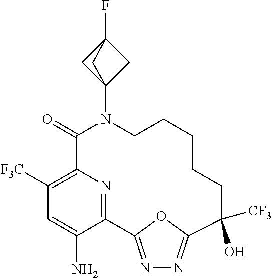 [SMILES: Nc1cc(C(F)(F)F)c2nc1-c1nnc(o1)[C@@](O)(C(F)(F)F)CCCCCN(C13CC(F)(C1)C3)C2=O] | +++ |
| 122 | 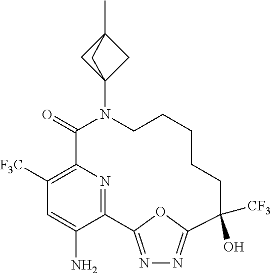 [SMILES: CC12CC(N3CCCCC[C@](O)(C(F)(F)F)c4nnc(o4)-c4nc(c(C(F)(F)F)cc4N)C3=O)(C1)C2] | +++ |
| 123 | 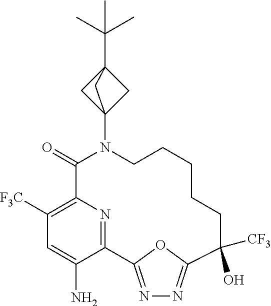 [SMILES: CC(C)(C)C12CC(N3CCCCC[C@](O)(C(F)(F)F)c4nnc(o4)-c4nc(c(C(F)(F)F)cc4N)C3=O)(C1)C2] | +++ |
| 124 | 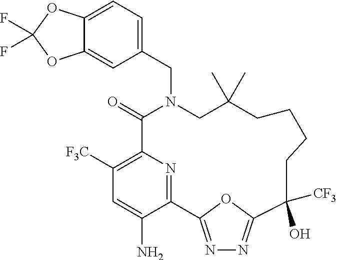 [SMILES: CC1(C)CCCC[C@](O)(C(F)(F)F)c2nnc(o2)-c2nc(c(C(F)(F)F)cc2N)C(=O)N(Cc2ccc3c(c2)OC(F)(F)O3)C1] | +++ |
| 125 | 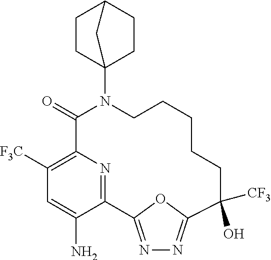 [SMILES: Nc1cc(C(F)(F)F)c2nc1-c1nnc(o1)[C@@](O)(C(F)(F)F)CCCCCN(C13CCC(CC1)C3)C2=O] | +++ |
| 126 | 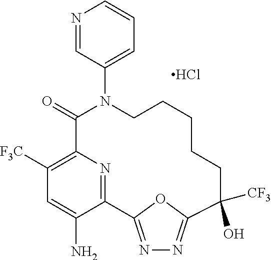 [SMILES: Nc1cc(C(F)(F)F)c2nc1-c1nnc(o1)[C@@](O)(C(F)(F)F)CCCCCN(c1cccnc1)C2=O ; Cl] | + |
| 127 | 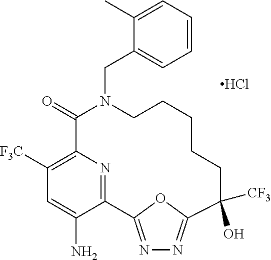 [SMILES: Cc1ccccc1CN1CCCCC[C@](O)(C(F)(F)F)c2nnc(o2)-c2nc(c(C(F)(F)F)cc2N)C1=O ; Cl] | +++ |
| 128 | 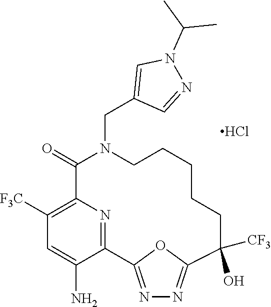 [SMILES: CC(C)n1cc(CN2CCCCC[C@](O)(C(F)(F)F)c3nnc(o3)-c3nc(c(C(F)(F)F)cc3N)C2=O)cn1 ; Cl] | +++ |
| 129 | 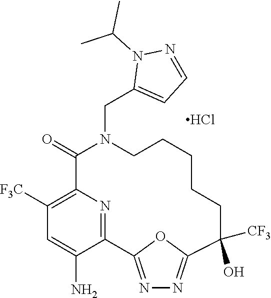 [SMILES: CC(C)n1nccc1CN1CCCCC[C@](O)(C(F)(F)F)c2nnc(o2)-c2nc(c(C(F)(F)F)cc2N)C1=O ; Cl] | +++ |
| 130 | 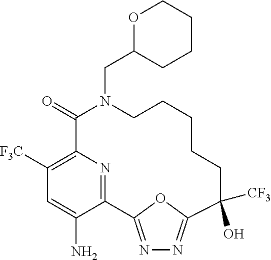 [SMILES: Nc1cc(C(F)(F)F)c2nc1-c1nnc(o1)[C@@](O)(C(F)(F)F)CCCCCN(CC1CCCCO1)C2=O] | +++ |
| 131 | 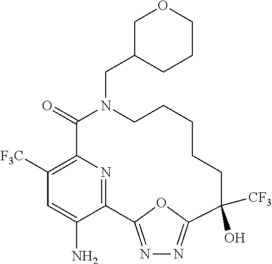 [SMILES: Nc1cc(C(F)(F)F)c2nc1-c1nnc(o1)[C@@](O)(C(F)(F)F)CCCCCN(CC1CCCOC1)C2=O] | +++ |
| 132 | 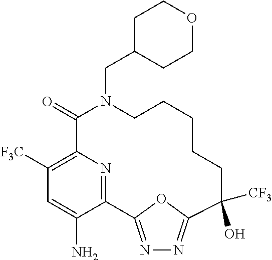 [SMILES: Nc1cc(C(F)(F)F)c2nc1-c1nnc(o1)[C@@](O)(C(F)(F)F)CCCCCN(CC1CCOCC1)C2=O] | +++ |
| 133 | 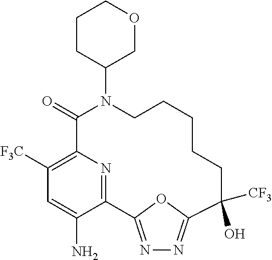 [SMILES: Nc1cc(C(F)(F)F)c2nc1-c1nnc(o1)[C@@](O)(C(F)(F)F)CCCCCN(C1CCCOC1)C2=O] | +++ |
| 134 | 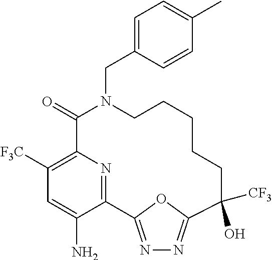 [SMILES: Cc1ccc(CN2CCCCC[C@](O)(C(F)(F)F)c3nnc(o3)-c3nc(c(C(F)(F)F)cc3N)C2=O)cc1] | +++ |
| 135 | 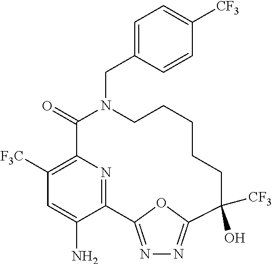 [SMILES: Nc1cc(C(F)(F)F)c2nc1-c1nnc(o1)[C@@](O)(C(F)(F)F)CCCCCN(Cc1ccc(C(F)(F)F)cc1)C2=O] | +++ |
| 136 | 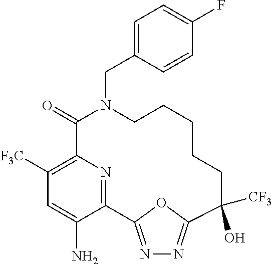 [SMILES: Nc1cc(C(F)(F)F)c2nc1-c1nnc(o1)[C@@](O)(C(F)(F)F)CCCCCN(Cc1ccc(F)cc1)C2=O] | +++ |
| 137 | 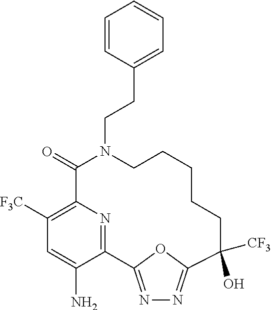 [SMILES: Nc1cc(C(F)(F)F)c2nc1-c1nnc(o1)[C@@](O)(C(F)(F)F)CCCCCN(CCc1ccccc1)C2=O] | +++ |
| 138 | 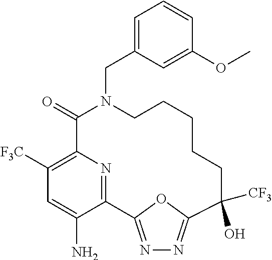 [SMILES: COc1cccc(CN2CCCCC[C@](O)(C(F)(F)F)c3nnc(o3)-c3nc(c(C(F)(F)F)cc3N)C2=O)c1] | +++ |
| 139 | 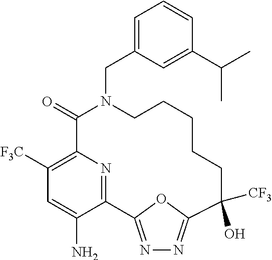 [SMILES: CC(C)c1cccc(CN2CCCCC[C@](O)(C(F)(F)F)c3nnc(o3)-c3nc(c(C(F)(F)F)cc3N)C2=O)c1] | +++ |
| 140 | 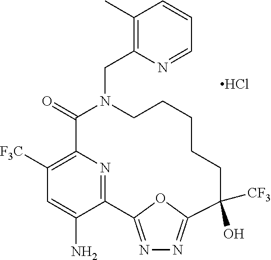 [SMILES: Cc1cccnc1CN1CCCCC[C@](O)(C(F)(F)F)c2nnc(o2)-c2nc(c(C(F)(F)F)cc2N)C1=O ; Cl] | +++ |
| 141 | 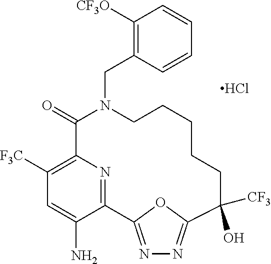 | +++ |
| 142 | 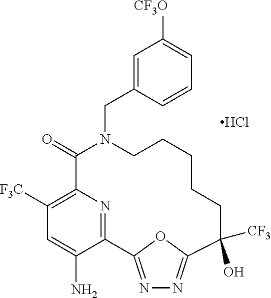 | +++ |
| 143 | 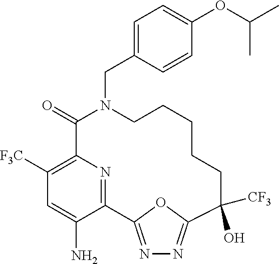 [SMILES: CC(C)Oc1ccc(CN2CCCCC[C@](O)(C(F)(F)F)c3nnc(o3)-c3nc(c(C(F)(F)F)cc3N)C2=O)cc1] | +++ |
| 144 | 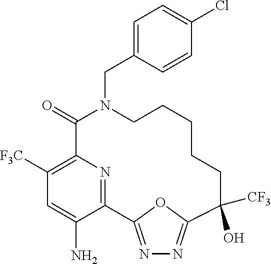 [SMILES: Nc1cc(C(F)(F)F)c2nc1-c1nnc(o1)[C@@](O)(C(F)(F)F)CCCCCN(Cc1ccc(Cl)cc1)C2=O] | +++ |
| 145 | 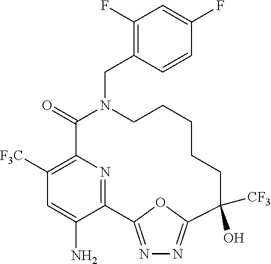 [SMILES: Nc1cc(C(F)(F)F)c2nc1-c1nnc(o1)[C@@](O)(C(F)(F)F)CCCCCN(Cc1ccc(F)cc1F)C2=O] | +++ |
| 146 | 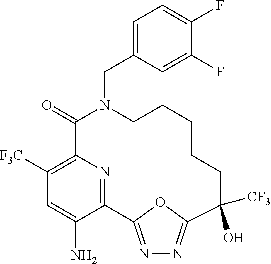 [SMILES: Nc1cc(C(F)(F)F)c2nc1-c1nnc(o1)[C@@](O)(C(F)(F)F)CCCCCN(Cc1ccc(F)c(F)c1)C2=O] | +++ |
| 147 | 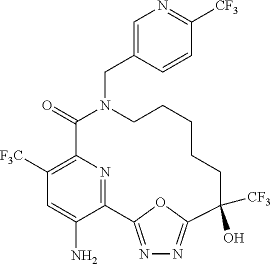 [SMILES: Nc1cc(C(F)(F)F)c2nc1-c1nnc(o1)[C@@](O)(C(F)(F)F)CCCCCN(Cc1ccc(C(F)(F)F)nc1)C2=O] | +++ |
| 148 | 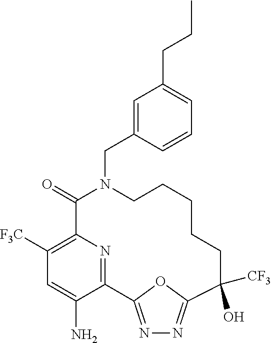 [SMILES: CCCc1cccc(CN2CCCCC[C@](O)(C(F)(F)F)c3nnc(o3)-c3nc(c(C(F)(F)F)cc3N)C2=O)c1] | +++ |
| 149 | 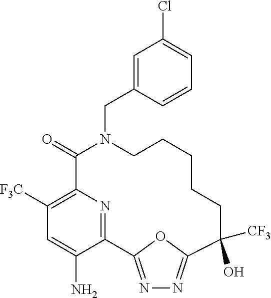 [SMILES: Nc1cc(C(F)(F)F)c2nc1-c1nnc(o1)[C@@](O)(C(F)(F)F)CCCCCN(Cc1cccc(Cl)c1)C2=O] | +++ |
| 150 | 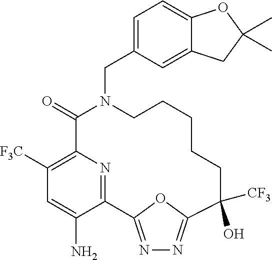 [SMILES: CC1(C)Cc2cc(CN3CCCCC[C@](O)(C(F)(F)F)c4nnc(o4)-c4nc(c(C(F)(F)F)cc4N)C3=O)ccc2O1] | +++ |
| 151 | 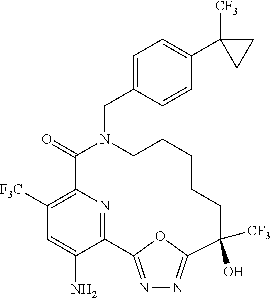 [SMILES: Nc1cc(C(F)(F)F)c2nc1-c1nnc(o1)[C@@](O)(C(F)(F)F)CCCCCN(Cc1ccc(C3(C(F)(F)F)CC3)cc1)C2=O] | +++ |
| 152 | 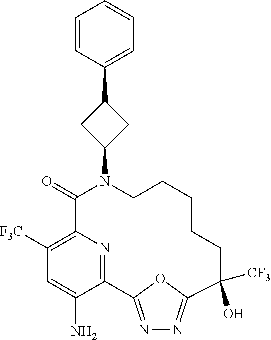 | +++ |
| 153 | 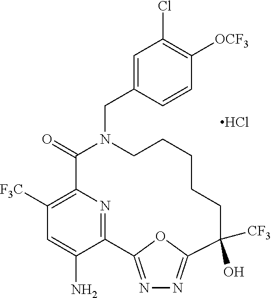 [SMILES: Nc1cc(C(F)(F)F)c2nc1-c1nnc(o1)[C@@](O)(C(F)(F)F)CCCCCN(Cc1ccc(OC(F)(F)F)c(Cl)c1)C2=O ; Cl] | +++ |
| 154 | 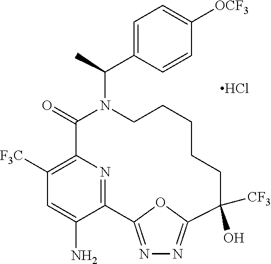 [SMILES: C[C@@H](c1ccc(OC(F)(F)F)cc1)N1CCCCC[C@](O)(C(F)(F)F)c2nnc(o2)-c2nc(c(C(F)(F)F)cc2N)C1=O ; Cl] | +++ |
| 155 | 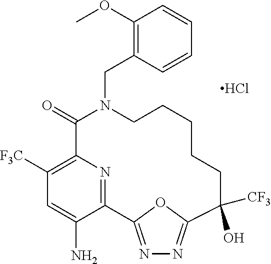 [SMILES: COc1ccccc1CN1CCCCC[C@](O)(C(F)(F)F)c2nnc(o2)-c2nc(c(C(F)(F)F)cc2N)C1=O ; Cl] | +++ |
| 156 | 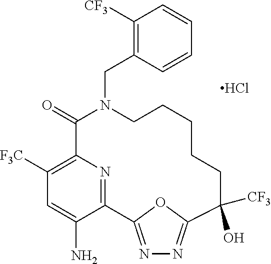 [SMILES: Nc1cc(C(F)(F)F)c2nc1-c1nnc(o1)[C@@](O)(C(F)(F)F)CCCCCN(Cc1ccccc1C(F)(F)F)C2=O ; Cl] | +++ |
| 157 | 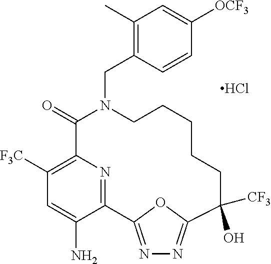 [SMILES: Cc1cc(OC(F)(F)F)ccc1CN1CCCCC[C@](O)(C(F)(F)F)c2nnc(o2)-c2nc(c(C(F)(F)F)cc2N)C1=O ; Cl] | +++ |
| 158 | 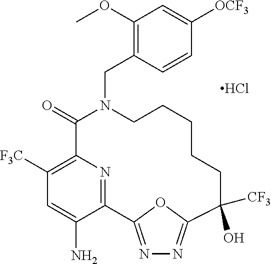 [SMILES: COc1cc(OC(F)(F)F)ccc1CN1CCCCC[C@](O)(C(F)(F)F)c2nnc(o2)-c2nc(c(C(F)(F)F)cc2N)C1=O ; Cl] | +++ |
| 159 | 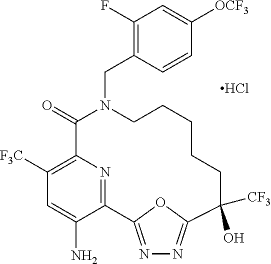 [SMILES: Nc1cc(C(F)(F)F)c2nc1-c1nnc(o1)[C@@](O)(C(F)(F)F)CCCCCN(Cc1ccc(OC(F)(F)F)cc1F)C2=O ; Cl] | +++ |
| 160 | 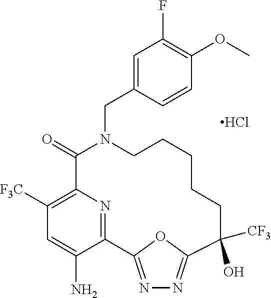 [SMILES: COc1ccc(CN2CCCCC[C@](O)(C(F)(F)F)c3nnc(o3)-c3nc(c(C(F)(F)F)cc3N)C2=O)cc1F ; Cl] | +++ |
| 161 | 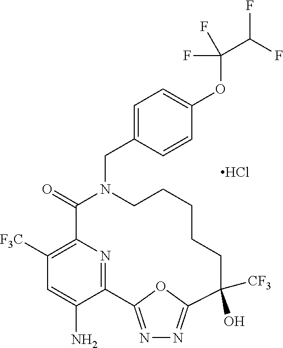 [SMILES: Nc1cc(C(F)(F)F)c2nc1-c1nnc(o1)[C@@](O)(C(F)(F)F)CCCCCN(Cc1ccc(OC(F)(F)C(F)F)cc1)C2=O ; Cl] | +++ |
| 162 | 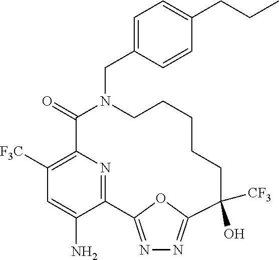 [SMILES: CCCc1ccc(CN2CCCCC[C@](O)(C(F)(F)F)c3nnc(o3)-c3nc(c(C(F)(F)F)cc3N)C2=O)cc1] | +++ |
| 163 | 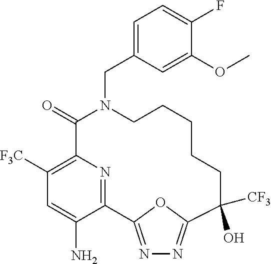 [SMILES: COc1cc(CN2CCCCC[C@](O)(C(F)(F)F)c3nnc(o3)-c3nc(c(C(F)(F)F)cc3N)C2=O)ccc1F] | +++ |
| 164 | 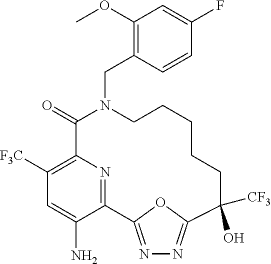 [SMILES: COc1cc(F)ccc1CN1CCCCC[C@](O)(C(F)(F)F)c2nnc(o2)-c2nc(c(C(F)(F)F)cc2N)C1=O] | +++ |
| 165 | 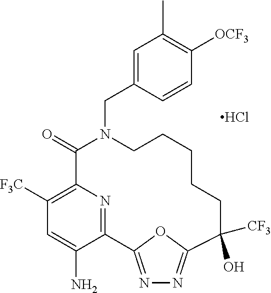 [SMILES: Cc1cc(CN2CCCCC[C@](O)(C(F)(F)F)c3nnc(o3)-c3nc(c(C(F)(F)F)cc3N)C2=O)ccc1OC(F)(F)F ; Cl] | +++ |
| 166 | 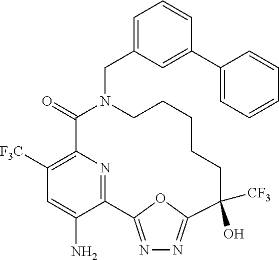 [SMILES: Nc1cc(C(F)(F)F)c2nc1-c1nnc(o1)[C@@](O)(C(F)(F)F)CCCCCN(Cc1cccc(-c3ccccc3)c1)C2=O] | +++ |
| 167 | 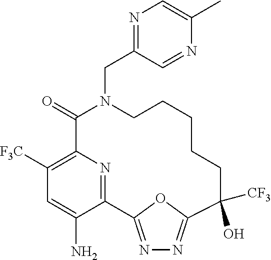 [SMILES: Cc1cnc(CN2CCCCC[C@](O)(C(F)(F)F)c3nnc(o3)-c3nc(c(C(F)(F)F)cc3N)C2=O)cn1] | + |
| 168 | 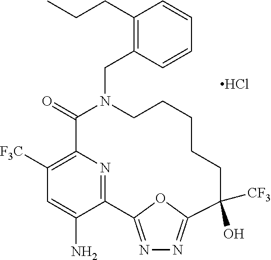 [SMILES: CCCc1ccccc1CN1CCCCC[C@](O)(C(F)(F)F)c2nnc(o2)-c2nc(c(C(F)(F)F)cc2N)C1=O ; Cl] | +++ |
| 169 | 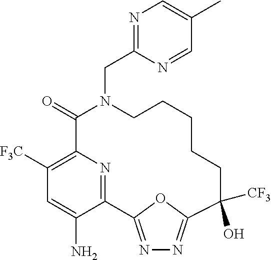 [SMILES: Cc1cnc(CN2CCCCC[C@](O)(C(F)(F)F)c3nnc(o3)-c3nc(c(C(F)(F)F)cc3N)C2=O)nc1] | + |
| 170 | 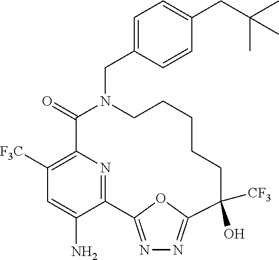 [SMILES: CC(C)(C)Cc1ccc(CN2CCCCC[C@](O)(C(F)(F)F)c3nnc(o3)-c3nc(c(C(F)(F)F)cc3N)C2=O)cc1] | +++ |
| 171 | 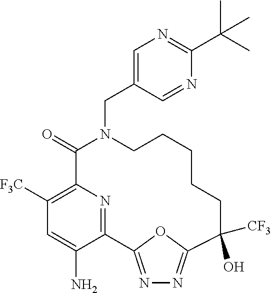 [SMILES: CC(C)(C)c1ncc(CN2CCCCC[C@](O)(C(F)(F)F)c3nnc(o3)-c3nc(c(C(F)(F)F)cc3N)C2=O)cn1] | +++ |
| 172 | 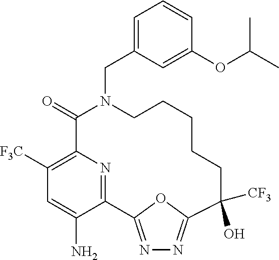 [SMILES: CC(C)Oc1cccc(CN2CCCCC[C@](O)(C(F)(F)F)c3nnc(o3)-c3nc(c(C(F)(F)F)cc3N)C2=O)c1] | +++ |
| 173 | 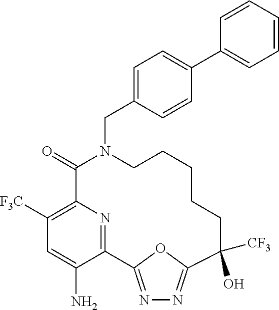 [SMILES: Nc1cc(C(F)(F)F)c2nc1-c1nnc(o1)[C@@](O)(C(F)(F)F)CCCCCN(Cc1ccc(-c3ccccc3)cc1)C2=O] | +++ |
| 174 | 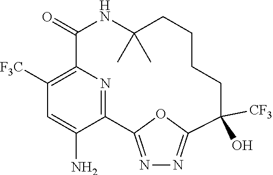 [SMILES: CC1(C)CCCC[C@](O)(C(F)(F)F)c2nnc(o2)-c2nc(c(C(F)(F)F)cc2N)C(=O)N1] | +++ |
| 175 | 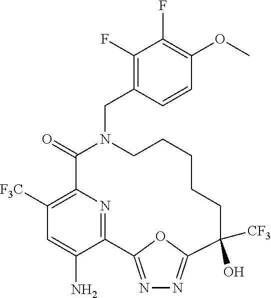 [SMILES: COc1ccc(CN2CCCCC[C@](O)(C(F)(F)F)c3nnc(o3)-c3nc(c(C(F)(F)F)cc3N)C2=O)c(F)c1F] | +++ |
| 176 | 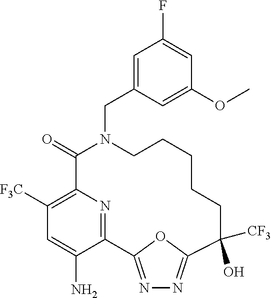 [SMILES: COc1cc(F)cc(CN2CCCCC[C@](O)(C(F)(F)F)c3nnc(o3)-c3nc(c(C(F)(F)F)cc3N)C2=O)c1] | +++ |
| 177 | 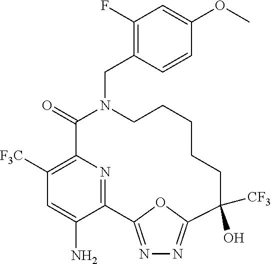 [SMILES: COc1ccc(CN2CCCCC[C@](O)(C(F)(F)F)c3nnc(o3)-c3nc(c(C(F)(F)F)cc3N)C2=O)c(F)c1] | +++ |
| 178 | 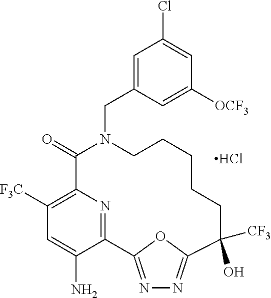 [SMILES: Nc1cc(C(F)(F)F)c2nc1-c1nnc(o1)[C@@](O)(C(F)(F)F)CCCCCN(Cc1cc(Cl)cc(OC(F)(F)F)c1)C2=O ; Cl] | +++ |
| 179 | 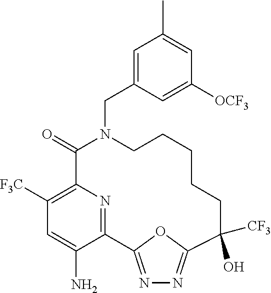 [SMILES: Cc1cc(CN2CCCCC[C@](O)(C(F)(F)F)c3nnc(o3)-c3nc(c(C(F)(F)F)cc3N)C2=O)cc(OC(F)(F)F)c1] | +++ |
| 180 | 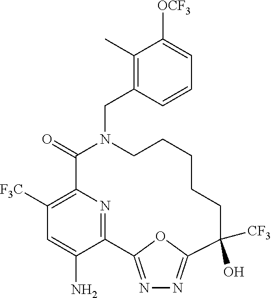 [SMILES: Cc1c(CN2CCCCC[C@](O)(C(F)(F)F)c3nnc(o3)-c3nc(c(C(F)(F)F)cc3N)C2=O)cccc1OC(F)(F)F] | +++ |
| 181 | 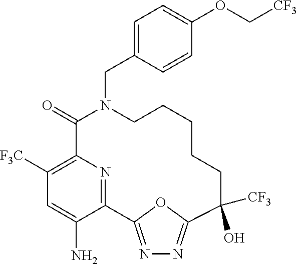 [SMILES: Nc1cc(C(F)(F)F)c2nc1-c1nnc(o1)[C@@](O)(C(F)(F)F)CCCCCN(Cc1ccc(OCC(F)(F)F)cc1)C2=O] | +++ |
| 182 | 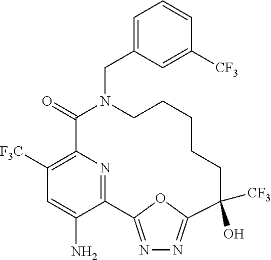 [SMILES: Nc1cc(C(F)(F)F)c2nc1-c1nnc(o1)[C@@](O)(C(F)(F)F)CCCCCN(Cc1cccc(C(F)(F)F)c1)C2=O] | +++ |
| 183 | 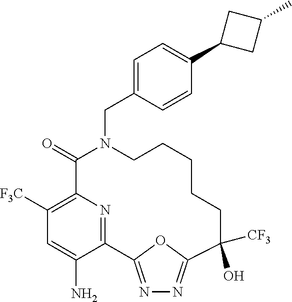 [SMILES: C[C@H]1C[C@H](c2ccc(CN3CCCCC[C@](O)(C(F)(F)F)c4nnc(o4)-c4nc(c(C(F)(F)F)cc4N)C3=O)cc2)C1] | +++ |
| 184 | 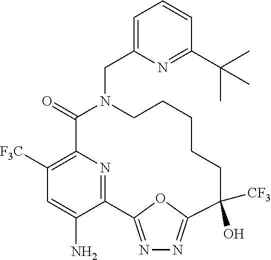 [SMILES: CC(C)(C)c1cccc(CN2CCCCC[C@](O)(C(F)(F)F)c3nnc(o3)-c3nc(c(C(F)(F)F)cc3N)C2=O)n1] | +++ |
| 185 | 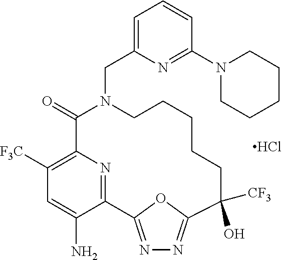 [SMILES: Nc1cc(C(F)(F)F)c2nc1-c1nnc(o1)[C@@](O)(C(F)(F)F)CCCCCN(Cc1cccc(N3CCCCC3)n1)C2=O ; Cl] | +++ |
| 186 | 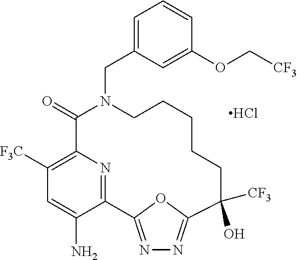 [SMILES: Nc1cc(C(F)(F)F)c2nc1-c1nnc(o1)[C@@](O)(C(F)(F)F)CCCCCN(Cc1cccc(OCC(F)(F)F)c1)C2=O ; Cl] | +++ |
| 187 | 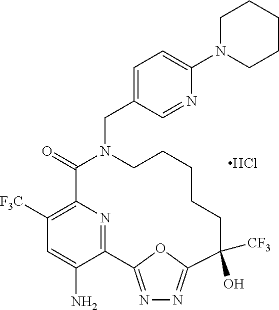 [SMILES: Nc1cc(C(F)(F)F)c2nc1-c1nnc(o1)[C@@](O)(C(F)(F)F)CCCCCN(Cc1ccc(N3CCCCC3)nc1)C2=O ; Cl] | +++ |
| 188 | 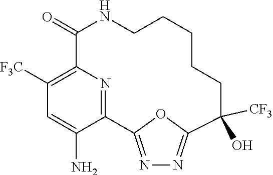 [SMILES: Nc1cc(C(F)(F)F)c2nc1-c1nnc(o1)[C@@](O)(C(F)(F)F)CCCCCNC2=O] | +++ |
| 189 | 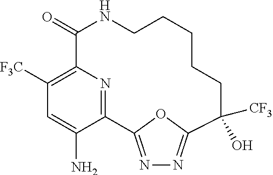 [SMILES: Nc1cc(C(F)(F)F)c2nc1-c1nnc(o1)[C@](O)(C(F)(F)F)CCCCCNC2=O] | + |
| 190 | 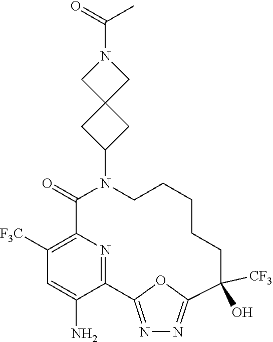 [SMILES: CC(=O)N1CC2(CC(N3CCCCC[C@](O)(C(F)(F)F)c4nnc(o4)-c4nc(c(C(F)(F)F)cc4N)C3=O)C2)C1] | + |
| 191 | 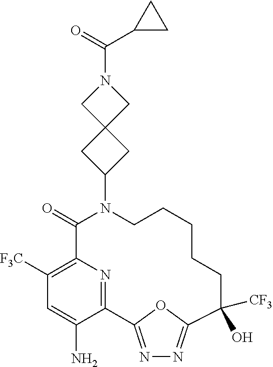 [SMILES: Nc1cc(C(F)(F)F)c2nc1-c1nnc(o1)[C@@](O)(C(F)(F)F)CCCCCN(C1CC3(C1)CN(C(=O)C1CC1)C3)C2=O] | + |
| 192 | 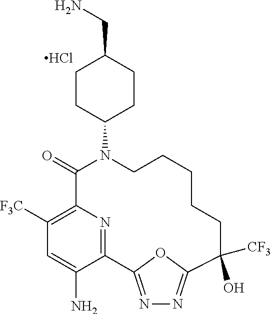 [SMILES: NC[C@H]1CC[C@H](N2CCCCC[C@](O)(C(F)(F)F)c3nnc(o3)-c3nc(c(C(F)(F)F)cc3N)C2=O)CC1 ; Cl] | + |
| 193 | 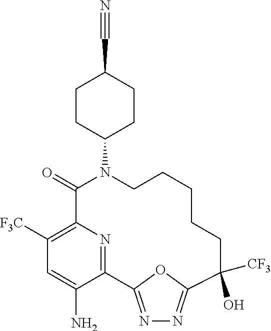 [SMILES: N#C[C@H]1CC[C@H](N2CCCCC[C@](O)(C(F)(F)F)c3nnc(o3)-c3nc(c(C(F)(F)F)cc3N)C2=O)CC1] | +++ |
| 194 | 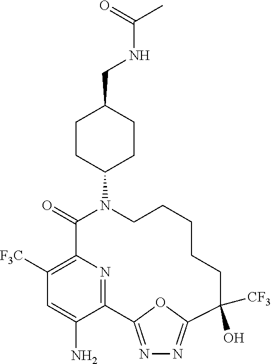 [SMILES: CC(=O)NC[C@H]1CC[C@H](N2CCCCC[C@](O)(C(F)(F)F)c3nnc(o3)-c3nc(c(C(F)(F)F)cc3N)C2=O)CC1] | ND |
| 195 | 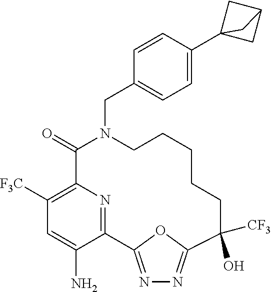 [SMILES: Nc1cc(C(F)(F)F)c2nc1-c1nnc(o1)[C@@](O)(C(F)(F)F)CCCCCN(Cc1ccc(C34CC(C3)C4)cc1)C2=O] | +++ |
| 196 | 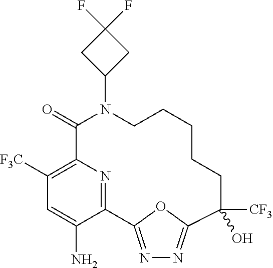 [SMILES: Nc1cc(C(F)(F)F)c2nc1-c1nnc(o1)C(O)(C(F)(F)F)CCCCCN(C1CC(F)(F)C1)C2=O] | +++ |
| 197 | 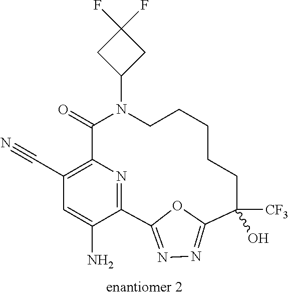 [SMILES: N#Cc1cc(N)c2nc1C(=O)N(C1CC(F)(F)C1)CCCCCC(O)(C(F)(F)F)c1nnc-2o1] | + |
| 198 | 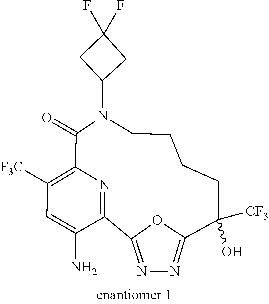 [SMILES: Nc1cc(C(F)(F)F)c2nc1-c1nnc(o1)C(O)(C(F)(F)F)CCCCN(C1CC(F)(F)C1)C2=O] | +++ |
| 199 | 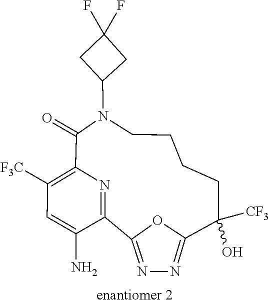 [SMILES: Nc1cc(C(F)(F)F)c2nc1-c1nnc(o1)C(O)(C(F)(F)F)CCCCN(C1CC(F)(F)C1)C2=O] | + |
| 200 | 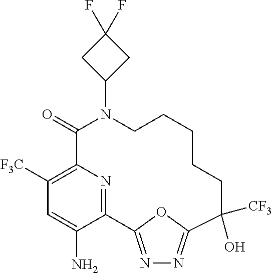 [SMILES: Nc1cc(C(F)(F)F)c2nc1-c1nnc(o1)C(O)(C(F)(F)F)CCCCCN(C1CC(F)(F)C1)C2=O] | ND |
| 201 | 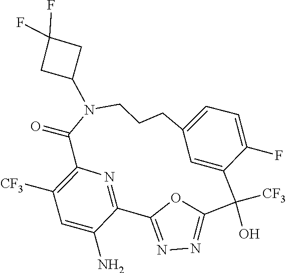 [SMILES: Nc1cc(C(F)(F)F)c2nc1-c1nnc(o1)C(O)(C(F)(F)F)c1cc(ccc1F)CCCN(C1CC(F)(F)C1)C2=O] | +++ |
| 202 | 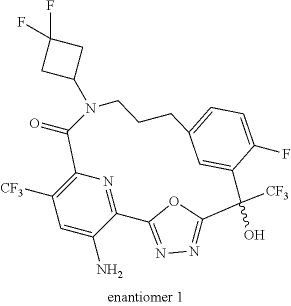 [SMILES: Nc1cc(C(F)(F)F)c2nc1-c1nnc(o1)C(O)(C(F)(F)F)c1cc(ccc1F)CCCN(C1CC(F)(F)C1)C2=O] | +++ |
| 203 | 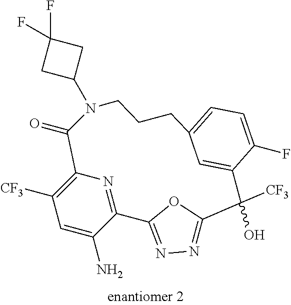 [SMILES: Nc1cc(C(F)(F)F)c2nc1-c1nnc(o1)C(O)(C(F)(F)F)c1cc(ccc1F)CCCN(C1CC(F)(F)C1)C2=O] | +++ |
| 204 | 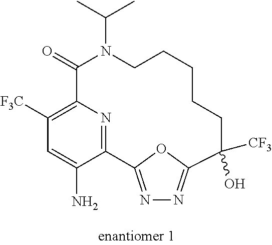 [SMILES: CC(C)N1CCCCCC(O)(C(F)(F)F)c2nnc(o2)-c2nc(c(C(F)(F)F)cc2N)C1=O] | +++ |
| 205 | 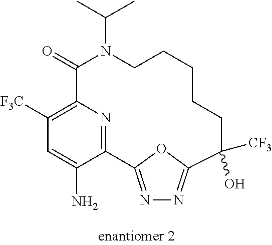 [SMILES: CC(C)N1CCCCCC(O)(C(F)(F)F)c2nnc(o2)-c2nc(c(C(F)(F)F)cc2N)C1=O] | + |
| 206 | 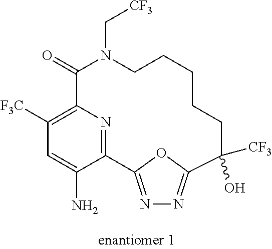 [SMILES: Nc1cc(C(F)(F)F)c2nc1-c1nnc(o1)C(O)(C(F)(F)F)CCCCCN(CC(F)(F)F)C2=O] | +++ |
| 207 | 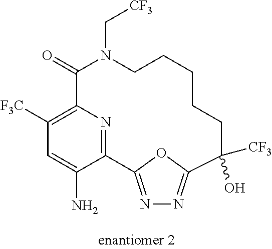 [SMILES: Nc1cc(C(F)(F)F)c2nc1-c1nnc(o1)C(O)(C(F)(F)F)CCCCCN(CC(F)(F)F)C2=O] | + |
| 208 | 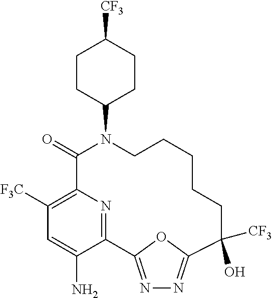 | +++ |
| 209 | 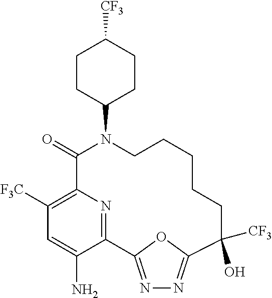 | +++ |
| 210 | 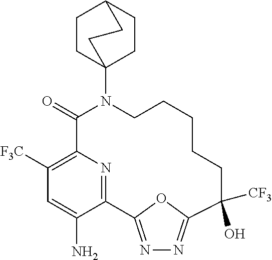 [SMILES: Nc1cc(C(F)(F)F)c2nc1-c1nnc(o1)[C@@](O)(C(F)(F)F)CCCCCN(C13CCC(CC1)CC3)C2=O] | +++ |
| 211 | 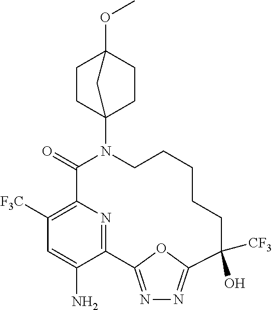 [SMILES: COC12CCC(N3CCCCC[C@](O)(C(F)(F)F)c4nnc(o4)-c4nc(c(C(F)(F)F)cc4N)C3=O)(CC1)C2] | +++ |
| 212 | 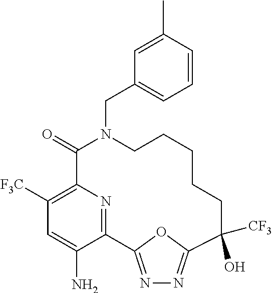 [SMILES: Cc1cccc(CN2CCCCC[C@](O)(C(F)(F)F)c3nnc(o3)-c3nc(c(C(F)(F)F)cc3N)C2=O)c1] | +++ |
| 213 | 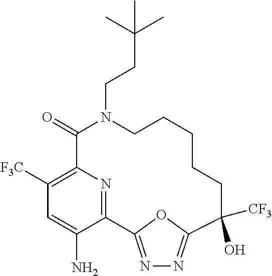 [SMILES: CC(C)(C)CCN1CCCCC[C@](O)(C(F)(F)F)c2nnc(o2)-c2nc(c(C(F)(F)F)cc2N)C1=O] | +++ |
| 214 | 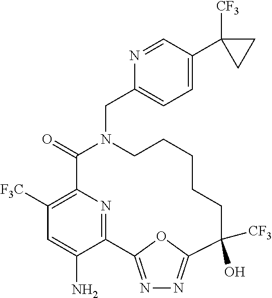 [SMILES: Nc1cc(C(F)(F)F)c2nc1-c1nnc(o1)[C@@](O)(C(F)(F)F)CCCCCN(Cc1ccc(C3(C(F)(F)F)CC3)cn1)C2=O] | +++ |
| 215 | 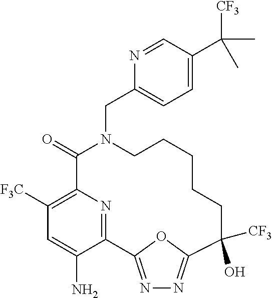 [SMILES: CC(C)(c1ccc(CN2CCCCC[C@](O)(C(F)(F)F)c3nnc(o3)-c3nc(c(C(F)(F)F)cc3N)C2=O)nc1)C(F)(F)F] | +++ |
| 216 | 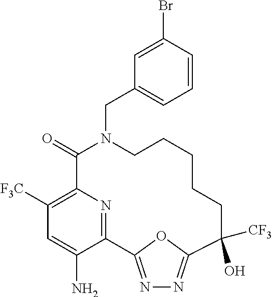 [SMILES: Nc1cc(C(F)(F)F)c2nc1-c1nnc(o1)[C@@](O)(C(F)(F)F)CCCCCN(Cc1cccc(Br)c1)C2=O] | +++ |
| 217 | 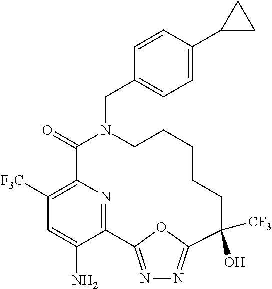 [SMILES: Nc1cc(C(F)(F)F)c2nc1-c1nnc(o1)[C@@](O)(C(F)(F)F)CCCCCN(Cc1ccc(C3CC3)cc1)C2=O] | +++ |
| 218 | 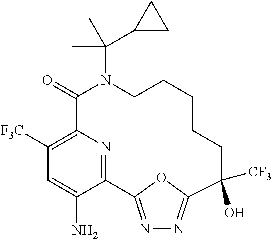 [SMILES: CC(C)(C1CC1)N1CCCCC[C@](O)(C(F)(F)F)c2nnc(o2)-c2nc(c(C(F)(F)F)cc2N)C1=O] | +++ |
| 219 | 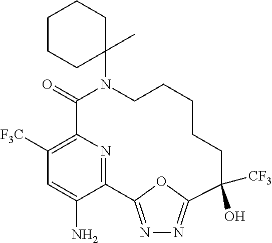 [SMILES: CC1(N2CCCCC[C@](O)(C(F)(F)F)c3nnc(o3)-c3nc(c(C(F)(F)F)cc3N)C2=O)CCCCC1] | +++ |
| 220 | 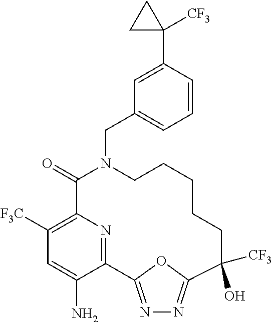 [SMILES: Nc1cc(C(F)(F)F)c2nc1-c1nnc(o1)[C@@](O)(C(F)(F)F)CCCCCN(Cc1cccc(C3(C(F)(F)F)CC3)c1)C2=O] | +++ |
| 221 | 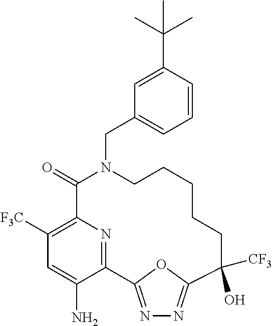 [SMILES: CC(C)(C)c1cccc(CN2CCCCC[C@](O)(C(F)(F)F)c3nnc(o3)-c3nc(c(C(F)(F)F)cc3N)C2=O)c1] | +++ |
| 222 | 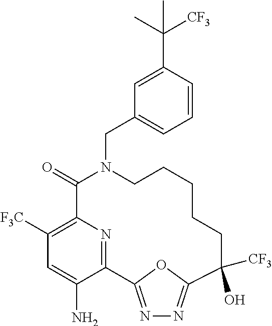 [SMILES: CC(C)(c1cccc(CN2CCCCC[C@](O)(C(F)(F)F)c3nnc(o3)-c3nc(c(C(F)(F)F)cc3N)C2=O)c1)C(F)(F)F] | +++ |
| 223 | 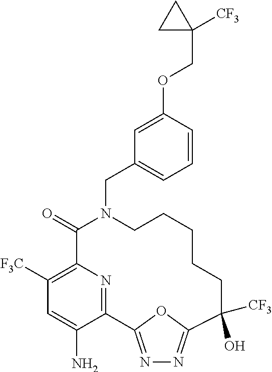 [SMILES: Nc1cc(C(F)(F)F)c2nc1-c1nnc(o1)[C@@](O)(C(F)(F)F)CCCCCN(Cc1cccc(OCC3(C(F)(F)F)CC3)c1)C2=O] | +++ |
| 224 | 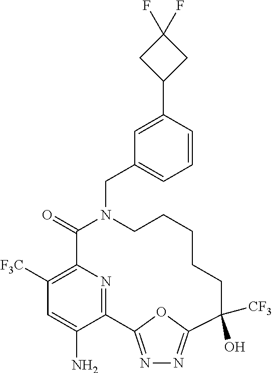 [SMILES: Nc1cc(C(F)(F)F)c2nc1-c1nnc(o1)[C@@](O)(C(F)(F)F)CCCCCN(Cc1cccc(C3CC(F)(F)C3)c1)C2=O] | +++ |
| 225 | 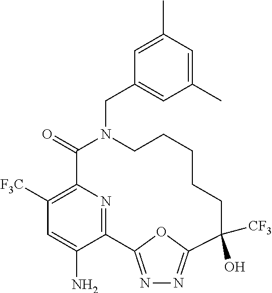 [SMILES: Cc1cc(C)cc(CN2CCCCC[C@](O)(C(F)(F)F)c3nnc(o3)-c3nc(c(C(F)(F)F)cc3N)C2=O)c1] | +++ |
| 226 | 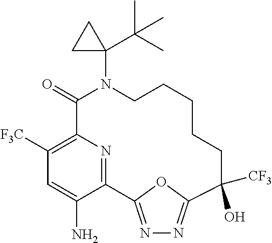 [SMILES: CC(C)(C)C1(N2CCCCC[C@](O)(C(F)(F)F)c3nnc(o3)-c3nc(c(C(F)(F)F)cc3N)C2=O)CC1] | +++ |
| 227 | 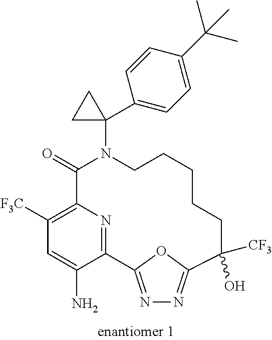 [SMILES: CC(C)(C)c1ccc(C2(N3CCCCCC(O)(C(F)(F)F)c4nnc(o4)-c4nc(c(C(F)(F)F)cc4N)C3=O)CC2)cc1] | +++ |
| 228 | 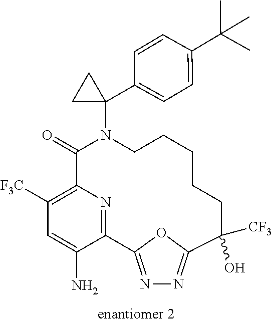 [SMILES: CC(C)(C)c1ccc(C2(N3CCCCCC(O)(C(F)(F)F)c4nnc(o4)-c4nc(c(C(F)(F)F)cc4N)C3=O)CC2)cc1] | +++ |
| 229 | 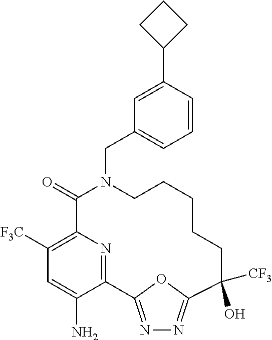 [SMILES: Nc1cc(C(F)(F)F)c2nc1-c1nnc(o1)[C@@](O)(C(F)(F)F)CCCCCN(Cc1cccc(C3CCC3)c1)C2=O] | +++ |
| 230 | 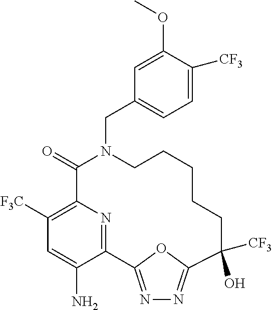 [SMILES: COc1cc(CN2CCCCC[C@](O)(C(F)(F)F)c3nnc(o3)-c3nc(c(C(F)(F)F)cc3N)C2=O)ccc1C(F)(F)F] | +++ |
| 231 | 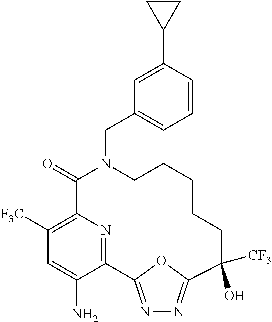 [SMILES: Nc1cc(C(F)(F)F)c2nc1-c1nnc(o1)[C@@](O)(C(F)(F)F)CCCCCN(Cc1cccc(C3CC3)c1)C2=O] | +++ |
| 232 | 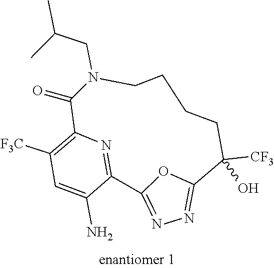 [SMILES: CC(C)CN1CCCCC(O)(C(F)(F)F)C2=NN=C(O2)c2nc(c(C(F)(F)F)cc2N)C1=O] | +++ |
| 233 | 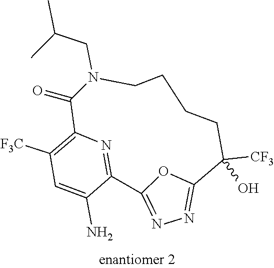 [SMILES: CC(C)CN1CCCCC(O)(C(F)(F)F)C2=NN=C(O2)c2nc(c(C(F)(F)F)cc2N)C1=O] | +++ |
| 234 | 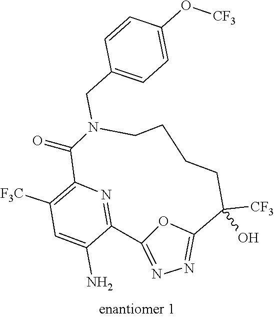 [SMILES: Nc1cc(C(F)(F)F)c2nc1C1=NN=C(O1)C(O)(C(F)(F)F)CCCCN(Cc1ccc(OC(F)(F)F)cc1)C2=O] | +++ |
| 235 | 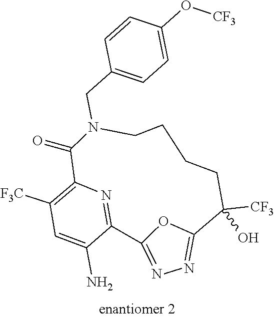 [SMILES: Nc1cc(C(F)(F)F)c2nc1C1=NN=C(O1)C(O)(C(F)(F)F)CCCCN(Cc1ccc(OC(F)(F)F)cc1)C2=O] | +++ |
| 236 | 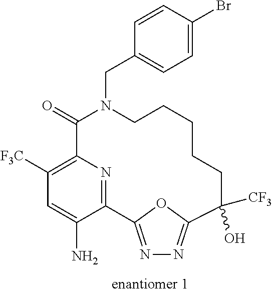 [SMILES: Nc1cc(C(F)(F)F)c2nc1-c1nnc(o1)C(O)(C(F)(F)F)CCCCCN(Cc1ccc(Br)cc1)C2=O] | +++ |
| 237 | 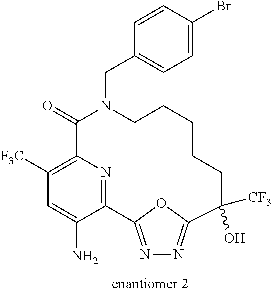 [SMILES: Nc1cc(C(F)(F)F)c2nc1-c1nnc(o1)C(O)(C(F)(F)F)CCCCCN(Cc1ccc(Br)cc1)C2=O] | ND |
| 238 | 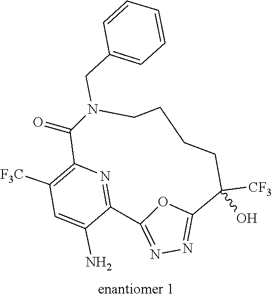 [SMILES: Nc1cc(C(F)(F)F)c2nc1C1=NN=C(O1)C(O)(C(F)(F)F)CCCCN(Cc1ccccc1)C2=O] | +++ |
| 239 | 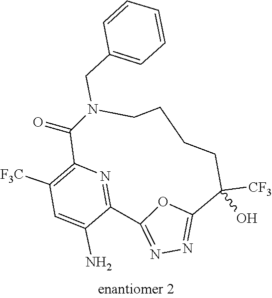 [SMILES: Nc1cc(C(F)(F)F)c2nc1C1=NN=C(O1)C(O)(C(F)(F)F)CCCCN(Cc1ccccc1)C2=O] | +++ |
| 240 | 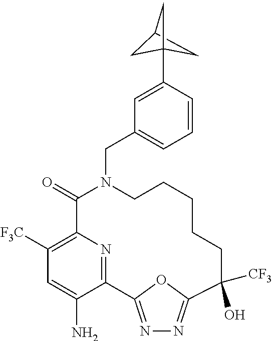 [SMILES: Nc1cc(C(F)(F)F)c2nc1-c1nnc(o1)[C@@](O)(C(F)(F)F)CCCCCN(Cc1cccc(C34CC(C3)C4)c1)C2=O] | +++ |
| 241 | 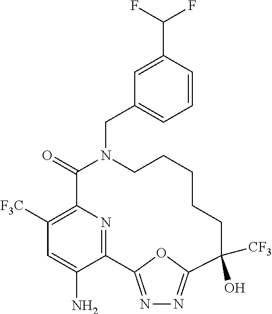 [SMILES: Nc1cc(C(F)(F)F)c2nc1-c1nnc(o1)[C@@](O)(C(F)(F)F)CCCCCN(Cc1cccc(C(F)F)c1)C2=O] | +++ |
| 242 | 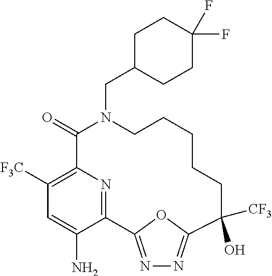 [SMILES: Nc1cc(C(F)(F)F)c2nc1-c1nnc(o1)[C@@](O)(C(F)(F)F)CCCCCN(CC1CCC(F)(F)CC1)C2=O] | +++ |
| 243 | 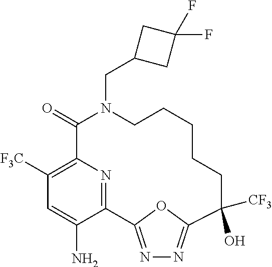 [SMILES: Nc1cc(C(F)(F)F)c2nc1-c1nnc(o1)[C@@](O)(C(F)(F)F)CCCCCN(CC1CC(F)(F)C1)C2=O] | +++ |
| 244 | 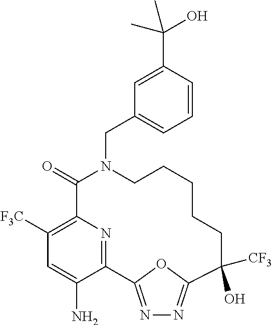 [SMILES: CC(C)(O)c1cccc(CN2CCCCC[C@](O)(C(F)(F)F)c3nnc(o3)-c3nc(c(C(F)(F)F)cc3N)C2=O)c1] | +++ |
| 245 | 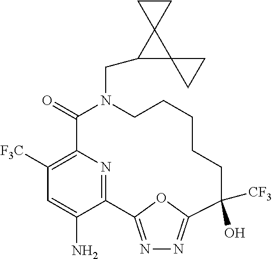 [SMILES: Nc1cc(C(F)(F)F)c2nc1-c1nnc(o1)[C@@](O)(C(F)(F)F)CCCCCN(CC1C3(CC3)C13CC3)C2=O] | +++ |
| 246 | 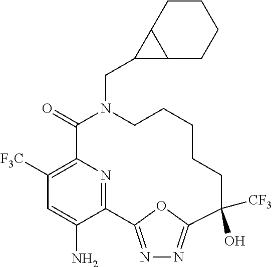 [SMILES: Nc1cc(C(F)(F)F)c2nc1-c1nnc(o1)[C@@](O)(C(F)(F)F)CCCCCN(CC1C3CCCCC31)C2=O] | +++ |
| 247 | 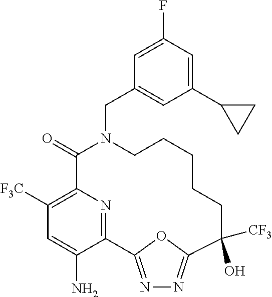 [SMILES: Nc1cc(C(F)(F)F)c2nc1-c1nnc(o1)[C@@](O)(C(F)(F)F)CCCCCN(Cc1cc(F)cc(C3CC3)c1)C2=O] | +++ |
| 248 | 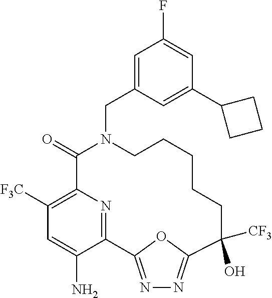 [SMILES: Nc1cc(C(F)(F)F)c2nc1-c1nnc(o1)[C@@](O)(C(F)(F)F)CCCCCN(Cc1cc(F)cc(C3CCC3)c1)C2=O] | +++ |
| 249 | 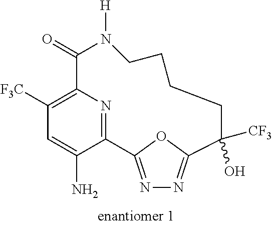 [SMILES: Nc1cc(C(F)(F)F)c2nc1-c1nnc(o1)C(O)(C(F)(F)F)CCCCNC2=O] | +++ |
| 250 | 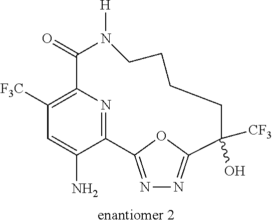 [SMILES: Nc1cc(C(F)(F)F)c2nc1-c1nnc(o1)C(O)(C(F)(F)F)CCCCNC2=O] | + |
| 251 | 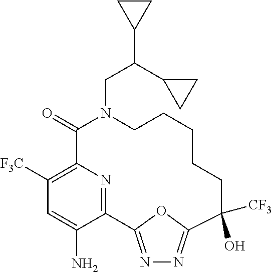 [SMILES: Nc1cc(C(F)(F)F)c2nc1-c1nnc(o1)[C@@](O)(C(F)(F)F)CCCCCN(CC(C1CC1)C1CC1)C2=O] | + |
| 252 | 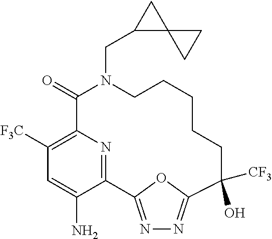 [SMILES: Nc1cc(C(F)(F)F)c2nc1-c1nnc(o1)[C@@](O)(C(F)(F)F)CCCCCN(CC1CC13CC3)C2=O] | +++ |
| 253 | 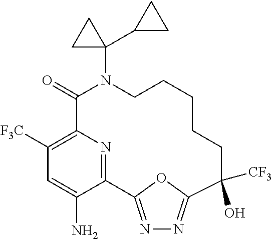 [SMILES: Nc1cc(C(F)(F)F)c2nc1-c1nnc(o1)[C@@](O)(C(F)(F)F)CCCCCN(C1(C3CC3)CC1)C2=O] | +++ |
| 254 | 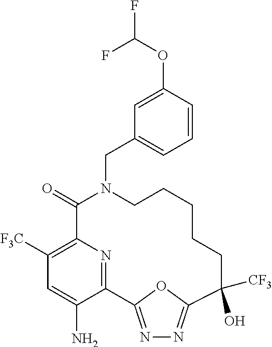 [SMILES: Nc1cc(C(F)(F)F)c2nc1-c1nnc(o1)[C@@](O)(C(F)(F)F)CCCCCN(Cc1cccc(OC(F)F)c1)C2=O] | +++ |
| 255 | 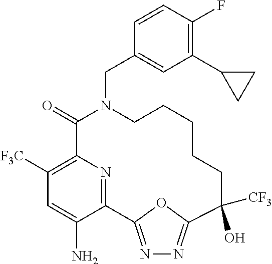 [SMILES: Nc1cc(C(F)(F)F)c2nc1-c1nnc(o1)[C@@](O)(C(F)(F)F)CCCCCN(Cc1ccc(F)c(C3CC3)c1)C2=O] | +++ |
| 256 | 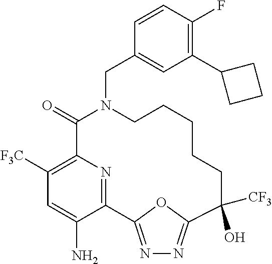 [SMILES: Nc1cc(C(F)(F)F)c2nc1-c1nnc(o1)[C@@](O)(C(F)(F)F)CCCCCN(Cc1ccc(F)c(C3CCC3)c1)C2=O] | +++ |
| 257 | 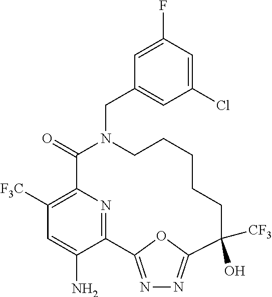 [SMILES: Nc1cc(C(F)(F)F)c2nc1-c1nnc(o1)[C@@](O)(C(F)(F)F)CCCCCN(Cc1cc(F)cc(Cl)c1)C2=O] | +++ |
| 258 | 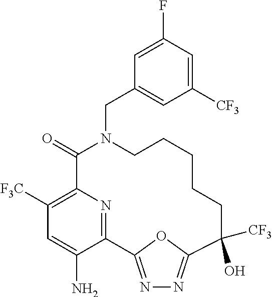 [SMILES: Nc1cc(C(F)(F)F)c2nc1-c1nnc(o1)[C@@](O)(C(F)(F)F)CCCCCN(Cc1cc(F)cc(C(F)(F)F)c1)C2=O] | +++ |
| 259 | 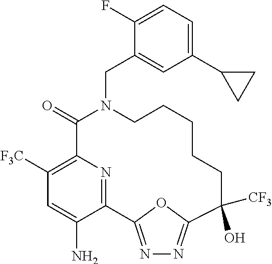 [SMILES: Nc1cc(C(F)(F)F)c2nc1-c1nnc(o1)[C@@](O)(C(F)(F)F)CCCCCN(Cc1cc(C3CC3)ccc1F)C2=O] | +++ |
| 260 | 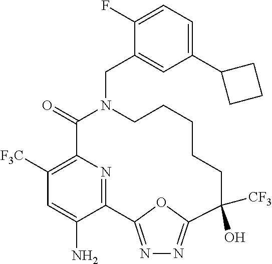 [SMILES: Nc1cc(C(F)(F)F)c2nc1-c1nnc(o1)[C@@](O)(C(F)(F)F)CCCCCN(Cc1cc(C3CCC3)ccc1F)C2=O] | +++ |
| 261 | 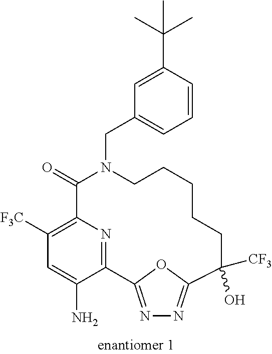 [SMILES: CC(C)(C)c1cccc(CN2CCCCCC(O)(C(F)(F)F)c3nnc(o3)-c3nc(c(C(F)(F)F)cc3N)C2=O)c1] | +++ |
| 262 | 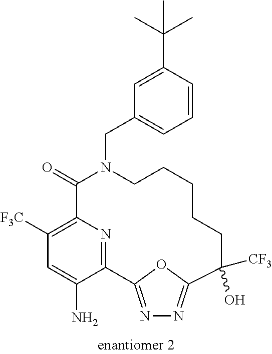 [SMILES: CC(C)(C)c1cccc(CN2CCCCCC(O)(C(F)(F)F)c3nnc(o3)-c3nc(c(C(F)(F)F)cc3N)C2=O)c1] | ND |
| 263 | 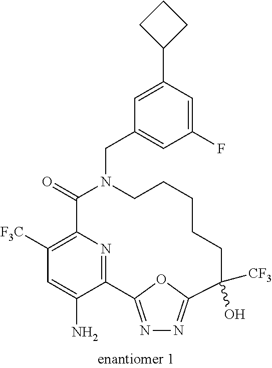 [SMILES: Nc1cc(C(F)(F)F)c2nc1-c1nnc(o1)C(O)(C(F)(F)F)CCCCCN(Cc1cc(F)cc(C3CCC3)c1)C2=O] | +++ |
| 264 | 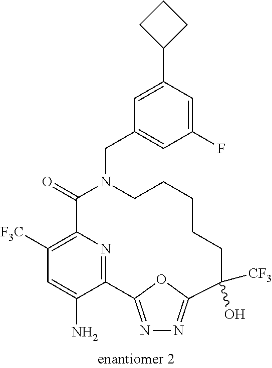 [SMILES: Nc1cc(C(F)(F)F)c2nc1-c1nnc(o1)C(O)(C(F)(F)F)CCCCCN(Cc1cc(F)cc(C3CCC3)c1)C2=O] | +++ |
| 265 | 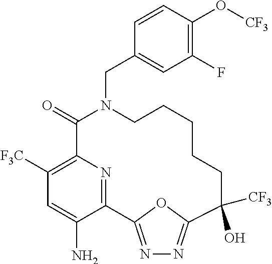 [SMILES: Nc1cc(C(F)(F)F)c2nc1-c1nnc(o1)[C@@](O)(C(F)(F)F)CCCCCN(Cc1ccc(OC(F)(F)F)c(F)c1)C2=O] | +++ |
| 266 | 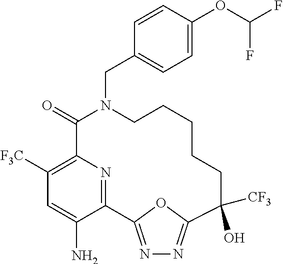 [SMILES: Nc1cc(C(F)(F)F)c2nc1-c1nnc(o1)[C@@](O)(C(F)(F)F)CCCCCN(Cc1ccc(OC(F)F)cc1)C2=O] | +++ |
| 267 | 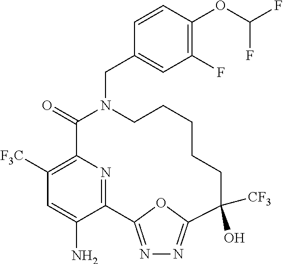 [SMILES: Nc1cc(C(F)(F)F)c2nc1-c1nnc(o1)[C@@](O)(C(F)(F)F)CCCCCN(Cc1ccc(OC(F)F)c(F)c1)C2=O] | +++ |
| 268 | 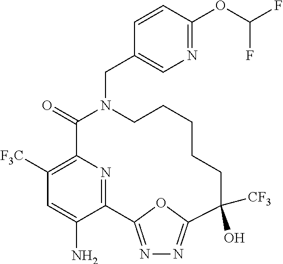 [SMILES: Nc1cc(C(F)(F)F)c2nc1-c1nnc(o1)[C@@](O)(C(F)(F)F)CCCCCN(Cc1ccc(OC(F)F)nc1)C2=O] | +++ |
| 269 | 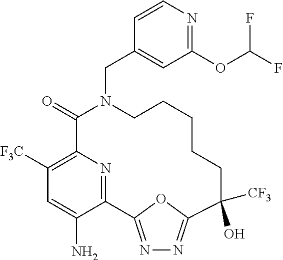 [SMILES: Nc1cc(C(F)(F)F)c2nc1-c1nnc(o1)[C@@](O)(C(F)(F)F)CCCCCN(Cc1ccnc(OC(F)F)c1)C2=O] | + |
| 270 | 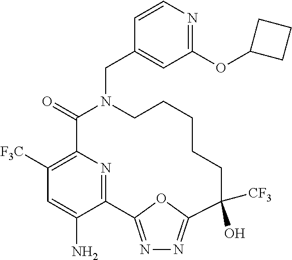 [SMILES: Nc1cc(C(F)(F)F)c2nc1-c1nnc(o1)[C@@](O)(C(F)(F)F)CCCCCN(Cc1ccnc(OC3CCC3)c1)C2=O] | +++ |
| 271 | 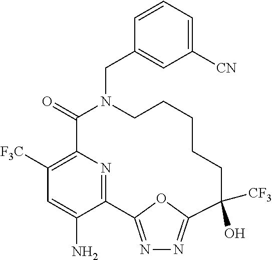 [SMILES: N#Cc1cccc(CN2CCCCC[C@](O)(C(F)(F)F)c3nnc(o3)-c3nc(c(C(F)(F)F)cc3N)C2=O)c1] | +++ |

### Other Embodiments

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A compound selected from compounds of Formula I: 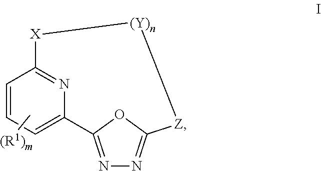
and deuterated derivatives and pharmaceutically acceptable salts thereof, wherein:
X is selected from —C(R^{X1})₂—, —CO—, 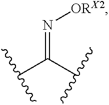
—Si(R^{Z3})₂—, and 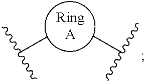
Ring A is cyclic group selected from phenyl and 5- to 6-membered heteroaryl, wherein the cyclic group is optionally substituted with 1-3 groups independently selected from C₁-C₆ alkyl;
each R^{X1} is independently selected from H, C₁-C₆ alkyl (optionally substituted with 1-3 groups independently selected from oxo, —OR^{X2}, and —N(R^{X2})₂), C₃-C₈ cycloalkyl, halogen, cyano, —OR^{X2}, and C₁-C₆ fluoroalkyl;
each R^{X2} is independently selected from H and C₁-C₆ alkyl;
each Y is independently selected from —C(R^{Y})₂—, —O—, —CO—, —NR^{YN}—, and 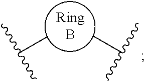
each R^{Y} is independently selected from hydrogen, hydroxy, halogen, C₁-C₆ alkyl (optionally substituted with 1-3 groups independently selected from hydroxy, C₁-C₆ alkoxy, and Q), C₃-C₈ cycloalkyl, C₆-C₁₀ aryl (optionally substituted with 1-3 groups independently selected from halogen), 5- to 10-membered heteroaryl, —OR^{Y1}, —CO₂R^{Y1}, —COR^{Y1}, —CON(R^{Y1})₂, and —N(R^{Y1})₂;
or two R^{Y}, one of which is on one atom and the second of which is on an adjacent atom, are taken together to form a pi bond;
each R^{Y1} is independently selected from hydrogen and C₁-C₆ alkyl, or two R^{Y1} bonded to the same nitrogen taken together form a 3- to 6-membered heterocyclyl;
each R^{YN} is independently selected from:
H,
C₁-C₆ alkyl, optionally substituted with 1-5 groups independently selected from:
oxo,
C₃-C₈ cycloalkyl (optionally substituted with 1-3 groups independently selected from halogen and C₁-C₆ alkyl),
C₆-C₁₀ aryl, optionally substituted with 1-3 groups independently selected from:
halogen,
cyano,
C₁-C₆ alkyl (optionally substituted with 1-3 hydroxy),
C₁-C₆ fluoroalkyl,
C₁-C₆ alkoxy (optionally substituted with C₃-C₈ cycloalkyl, which is optionally substituted with C₁-C₆ fluoroalkyl),
C₁-C₆ fluoroalkoxy,
C₃-C₈ cycloalkyl (optionally substituted with 1-3 groups independently selected from halogen, C₁-C₆ alkyl, and C₁-C₆ fluoroalkyl), and
phenyl (optionally substituted with 1-3 groups independently selected from C₁-C₆ alkyl, C₁-C₆ alkoxy, C₁-C₆ fluoroalkyl, C₁-C₆ fluoroalkoxy, and halogen),
5- to 10-membered heteroaryl, optionally substituted with 1-3 groups independently selected from:
halogen,
C₁-C₆ alkyl,
C₁-C₆ fluoroalkyl,
C₁-C₆ fluoroalkoxy,
—O₍₀₋₁₎(C₃-C₈ cycloalkyl) (optionally substituted with C₁-C₆ fluoroalkyl), and
3- to 12-membered heterocyclyl, and
3- to 12-membered heterocyclyl, optionally substituted with 1-3 groups selected from halogen and C₁-C₆ alkyl)
C₁-C₆ fluoroalkyl,
C₆-C₁₀ aryl,
C₃-C₈ cycloalkyl optionally substituted with 1-3 groups independently selected from
halogen,
cyano,
C₁-C₄ alkyl optionally substituted with 1-3 groups selected from —N(R^{YN1})₂,
C₁-C₆ fluoroalkyl,
C₁-C₆ alkoxy,
C₃-C₈ cycloalkyl, and
phenyl (optionally substituted with 1-3 groups independently selected from C₁-C₆ alkyl)
3- to 12-membered heterocyclyl, optionally substituted with 1-3 groups selected from oxo and C₁-C₄ alkyl (optionally substituted with 1-2 groups selected from oxo and C₃-C₈ cycloalkyl),
5- to 6-membered heteroaryl (optionally substituted with 1-3 groups selected from C₁-C₆ alkyl), and
CO₂R^{YN1},
each R^{YN}1 is independently selected from H, C₁-C₄ alkyl (optionally substituted with oxo), and C₃-C₆ cycloalkyl;
Ring B is selected from:
C₆-C₁₀ aryl (optionally substituted with 1-3 groups independently selected from halogen, C₁-C₆ alkyl, and C₁-C₆ alkoxy),
C₃-C₈ cycloalkyl,
5- to 10-membered heteroaryl, and
3- to 6-membered heterocyclyl (optionally substituted with 1-3 groups independently selected from C₁-C₆ alkyl);
each Q is independently selected from:
C₁-C₆ alkyl optionally substituted with 1-3 groups independently selected from:
halogen,
oxo,
C₆-C₁₀ aryl (optionally substituted with 1-3 groups independently selected from halogen and —OCF₃), and
C₃-C₈ cycloalkyl,
C₃-C₈ cycloalkyl optionally substituted with 1-3 groups independently selected from:
halogen,
CN,
C₁-C₆ alkyl (optionally substituted with 1-3 groups independently selected from halogen, —NH₂, and —NHCOMe),
C₁-C₆ alkoxy,
C₆-C₁₀ aryl (optionally substituted with 1-3 groups independently selected from C₁-C₆ alkyl), and
C₃-C₈ cycloalkyl,
C₆-C₁₀ aryl optionally substituted with 1-3 groups independently selected from:
halogen,
CN,
C₁-C₆ alkyl (optionally substituted with 1-3 groups independently selected from halogen and hydroxy),
C₁-C₆ alkoxy optionally substituted with 1-4 groups independently selected from:
halogen,
C₃-C₈ cycloalkyl (optionally substituted with CF₃),
C₃-C₈ cycloalkyl (optionally substituted with 1-3 groups independently selected from halogen, CF₃, OCF₃, and C₁-C₆ alkyl), and
C₆-C₁₀ aryl,
5- to 10-membered heteroaryl optionally substituted with 1-3 groups independently selected from:
halogen,
C₁-C₆ alkyl (optionally substituted with 1-3 groups independently selected from halogen),
C₃-C₈ cycloalkyl (optionally substituted with 1-3 CF₃ groups), and
3- to 10-membered heterocyclyl,
3- to 10-membered heterocyclyl optionally substituted with 1-3 groups independently selected from:
C₁-C₆ alkyl (optionally substituted with 1-3 groups independently selected from oxo and C₃-C₈ cycloalkyl), and
oxo;
each R¹ is independently selected from halogen, C₁-C₆ fluoroalkyl, C₁-C₆ alkyl (optionally substituted with a group selected from hydroxy, C₆-C₁₀ aryl, and 5- to 6-membered heteroaryl), —OR², —N(R²)₂, —CO₂R², —CO—N(R²)₂, —CN, C₃-C₈ cycloalkyl, C₆-C₁₀ aryl, 5- to 6-membered heteroaryl (optionally substituted with 1-3 groups independently selected from C₁-C₆ alkyl), 3- to 6-membered heterocyclyl, —B(OR²)₂, —SO₂R², —SR², —SOR², —PO(OR²)₂, and —PO(R²)₂;
each R² is independently selected from hydrogen, C₁-C₆ alkyl (optionally substituted with 1-6 groups independently selected from halogen), C₁-C₆ fluoroalkyl, and C₆-C₁₀ aryl (optionally substituted with 1-3 groups independently selected from C₁-C₆ fluoroalkyl and C₁-C₆ fluoroalkoxy);
Z is selected from 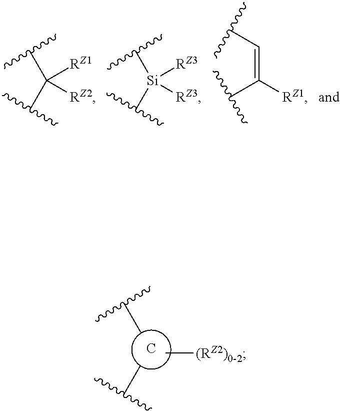
wherein Ring C is selected from C₆-C₁₀ aryl and 5- to 10-membered heteroaryl;
R^{Z1} is selected from hydrogen, —CN, C₁-C₆ alkyl (optionally substituted with 1-3 hydroxy), C₁-C₆ fluoroalkyl, 3- to 6-membered heterocyclyl, C₃-C₆ cycloalkyl, C₆-C₁₀ aryl, and 5- to 6-membered heteroaryl;
R^{Z2} is selected from hydrogen, halogen, hydroxy, NH₂, NH(CO)(C₁-C₆ alkyl), and C₁-C₆ alkoxy (optionally substituted with 1-3 groups independently selected from C₃-C₁₀ cycloalkyl),
or R^{Z1} and R^{Z2} taken together form a group selected from oxo and ═N—OH;
each R^{Z3} is independently selected from hydroxy, C₁-C₆ alkoxy, C₁-C₆ alkyl, and C₆-C₁₀ aryl; or two R^{Z3} are taken together to form a 3- to 6-membered heterocyclyl;
n is selected from 4, 5, 6, 7, and 8; and
m is selected from 0, 1, 2, and 3.

2. The compound, deuterated derivative or pharmaceutically acceptable salt according to claim 1, wherein Ring A is a cyclic group selected from phenyl, pyrazole, and oxadiazole.

3. The compound, deuterated derivative, or pharmaceutically acceptable salt according to claim 1 or claim 2, wherein each R^{X1} is independently selected from H, C₁-C₆ alkyl, halogen, cyano, —OR^{X2}, and C₁-C₆ fluoroalkyl.

4. The compound, deuterated derivative, or pharmaceutically acceptable salt according to any one of claims 1-3, wherein each R^{X2} is independently selected from H and C₁-C₄ alkyl.

5. The compound, deuterated derivative, or pharmaceutically acceptable salt according to any one of claims 1-4, wherein each R^{X1} is independently selected from H, F, —CF₃, —CH₃, —OH, —OCH₃, and CN.

6. The compound, deuterated derivative, or pharmaceutically acceptable salt according to any one of claims 1-5, wherein X is selected from: —CO—, —CH₂—,

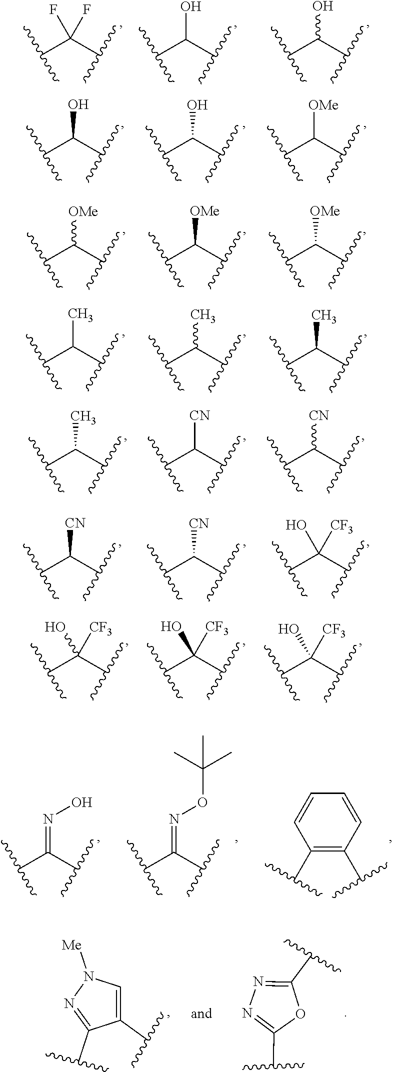

7. The compound, deuterated derivative, or pharmaceutically acceptable salt according to any one of claims 1-6, wherein each Y is independently selected from —C(R^{Y})₂—, —CO—, —NR^{YN}—, and

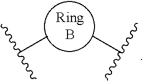

8. The compound, deuterated derivative, or pharmaceutically acceptable salt according to any one of claims 1-7, wherein each R^{Y} is independently selected from hydrogen, hydroxy, halogen, and C₁-C₆ alkyl.

9. The compound, deuterated derivative, or pharmaceutically acceptable salt according to any one of claims 1-8, wherein each R^{Y} is independently selected from H, —OH, —F, and —CH₃.

10. The compound, deuterated derivative, or pharmaceutically acceptable salt according to any one of claims 1-9, wherein each R^{YN} is independently selected from:
H,
—CH₂CF₃,
C₁-C₆ alkyl, optionally substituted with 1-3 groups independently selected from:
oxo,
cyclopropyl,
cyclobutyl optionally substituted with 1-2 fluoro groups,
cyclohexyl optionally substituted with 1-2 fluoro groups,
spiro[2.2]pentane,
bicyclo[4.1.0]heptane,
dispiro[2.0.24.13]heptane,
phenyl optionally substituted with 1-3 groups independently selected from F, Cl, Br, CN, methyl, propyl (optionally substituted with one hydroxy), tert-butyl, neopentyl, —CHF₂, —CF₃, —C(CH₃)₂CF₃, —OCH₃, —O(2-propyl), —OCHF₂, —OCF₃, —OCF₂CHF₂, —OCH₂CF₃, cyclopropyl (optionally substituted with one CF₃), cyclobutyl (optionally substituted with 1-2 groups selected from fluoro and methyl), spiro[2.2]pentane, bicyclo[1.1.1]pentane, bicyclo[4.1.0]heptane, dispiro[2.0.24.13]heptane, phenyl, naphthyl, and 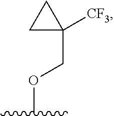 [SMILES: *OCC1(C(F)(F)F)CC1]
naphthyl,
2,2-dimethyl-2,3-dihydrobenzofuran,
2,2-difluorobenzo[d][1,3]dioxole,
tetrahydro-2H-pyran,
pyrazole optionally substituted with one group selected from methyl and propyl,
pyridine optionally substituted with one group selected from methyl, tert-butyl, CF₃, —O(cyclobutyl), —OCHF₂, cyclopropyl optionally substituted with one CF₃, and piperidine, and
pyrimidine optionally substituted with one group selected from methyl and tert-butyl,
cyclopropyl optionally substituted with 1-2 groups selected from methyl, tert-butyl, cyclopropyl, and phenyl (optionally substituted with one tert-butyl group),
cyclobutyl optionally substituted with 1-3 groups selected from fluoro, methyl, and phenyl,
cyclopentyl,
cyclohexyl optionally substituted with 1-2 groups selected from fluoro, methyl, tert-butyl, cyano, —CF₃, —CH₂NH₂, and —CH₂NHAc,
bicyclo[1.1.1]pentane optionally substituted by one group selected from fluoro, methyl, and tert-butyl,
bicyclo[2.2.1]heptane optionally substituted with one —OCH₃ group,
bicyclo[2.2.2]octane,
spiro[3.3]heptane optionally substituted with 1-2 fluoro groups,
phenyl,
tetrahydro-2H-pyran,
2-azaspiro[3.3]heptane optionally substituted with one group selected from —COMe, and —CO(cyclopropyl),
thietane 1,1-dioxide,
tetrahydro-2H-thiopyran 1,1-dioxide,
pyrazole optionally substituted with one methyl group, and
pyridine.

11. The compound according to any one of claims 1-10, wherein each R^{YN} is independently selected from: H, —CH₃,

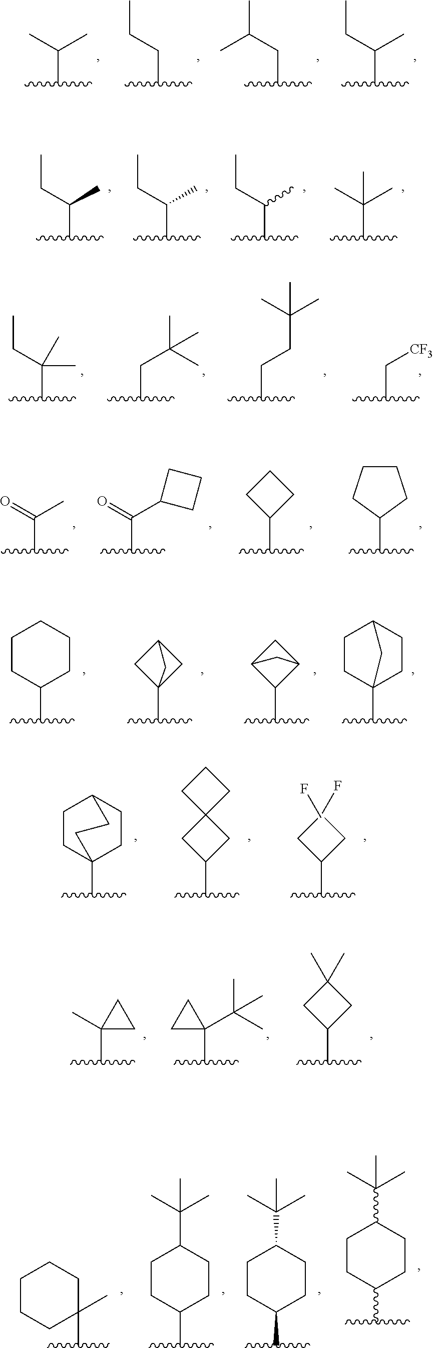

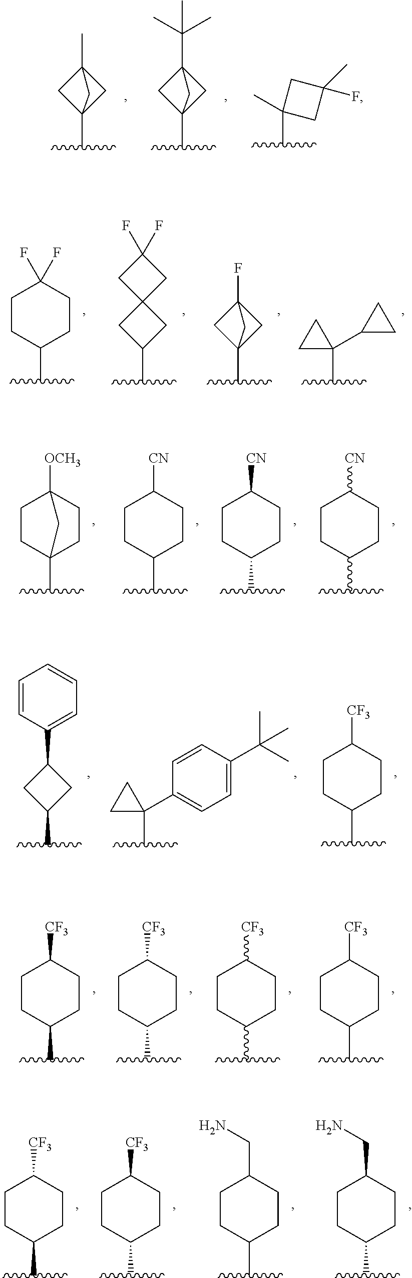

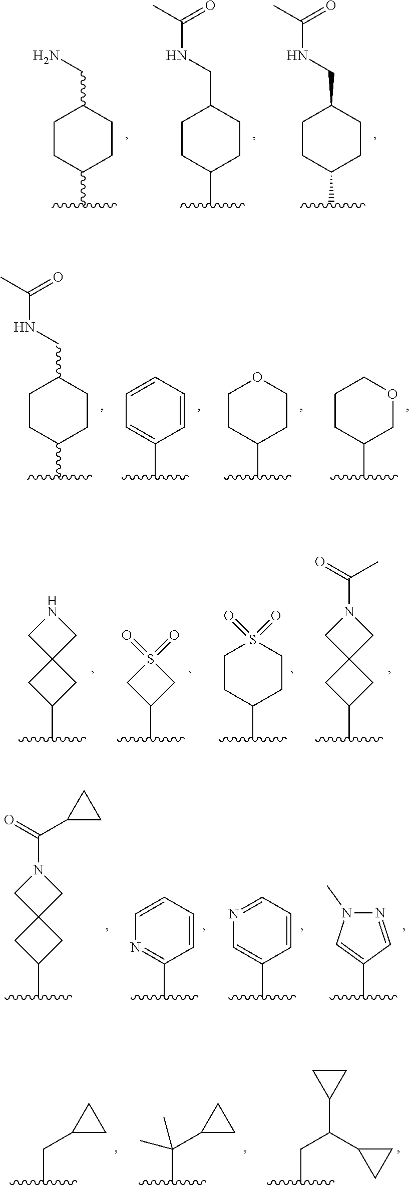

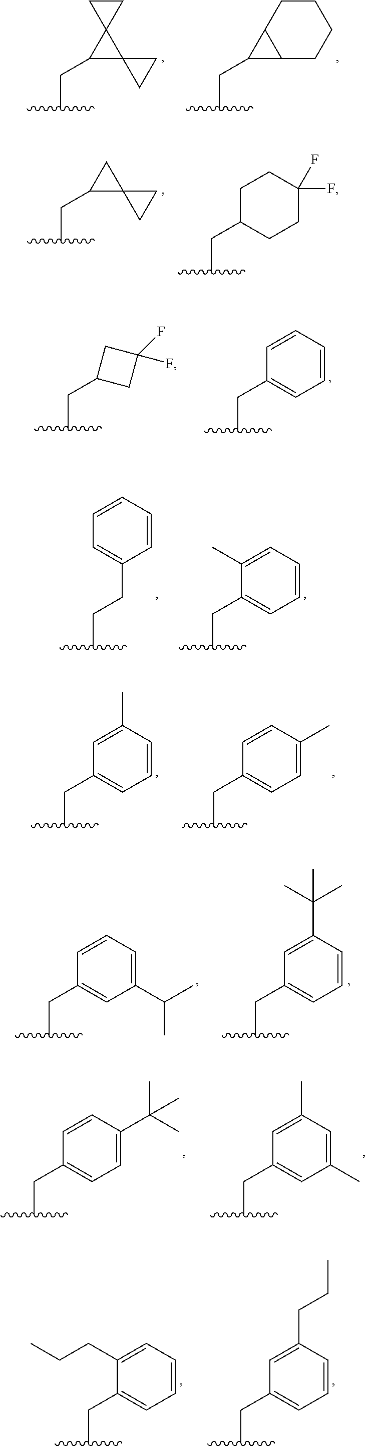

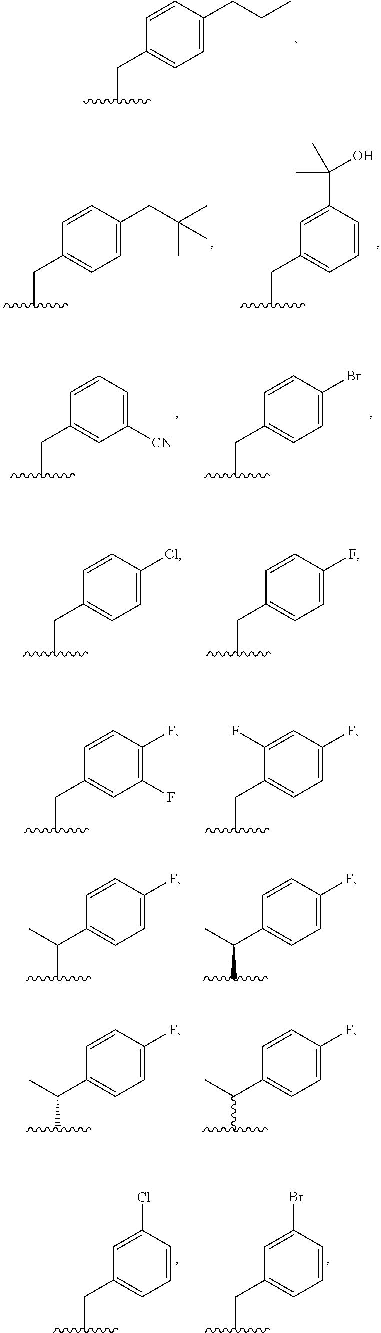

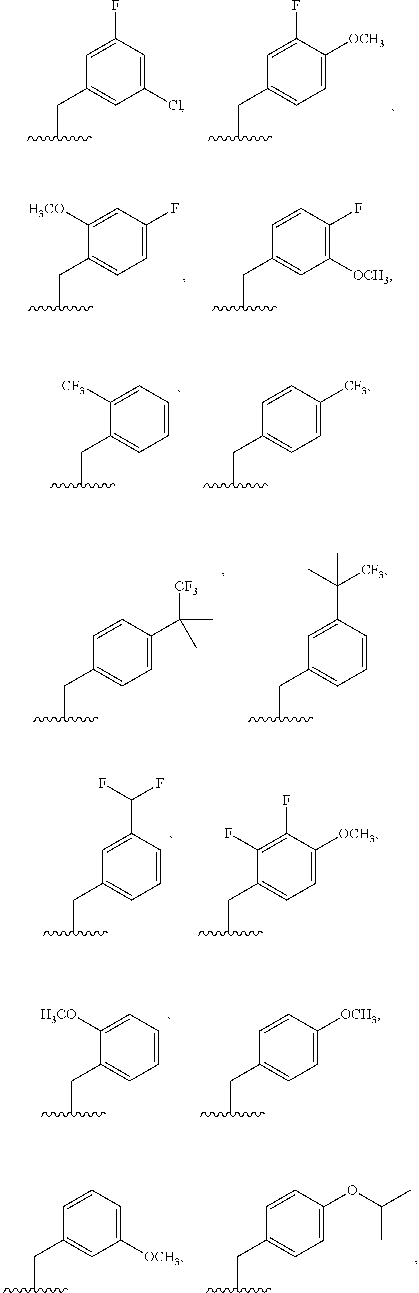

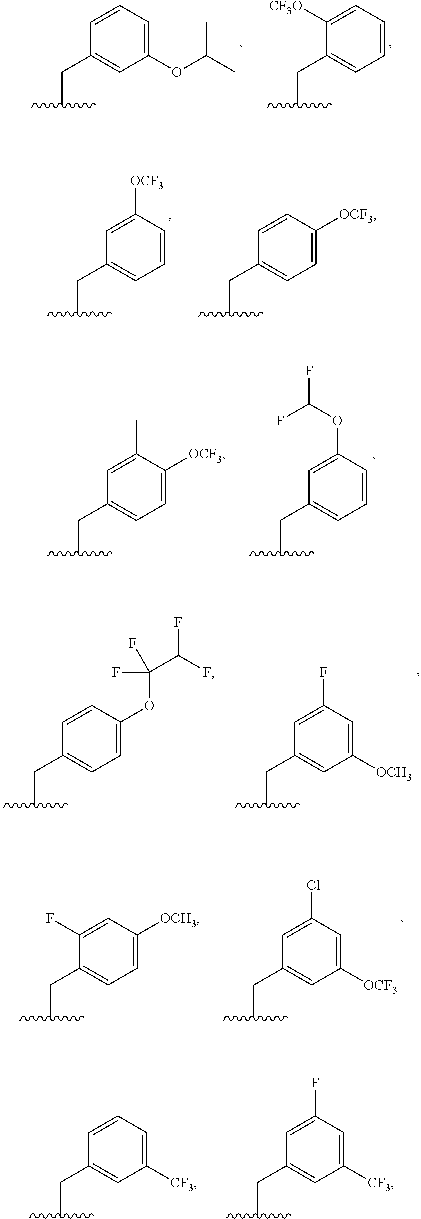

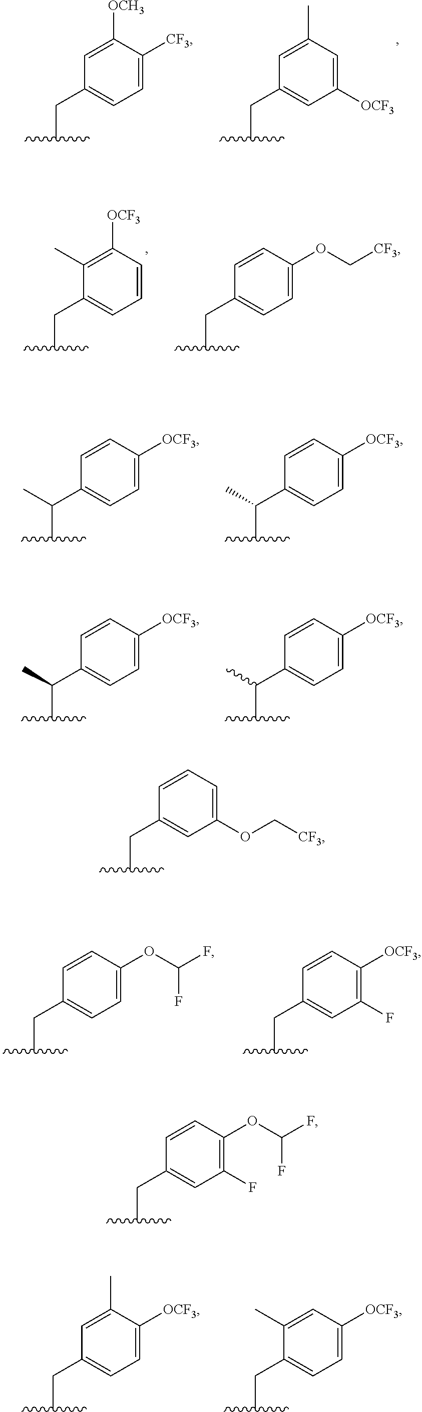

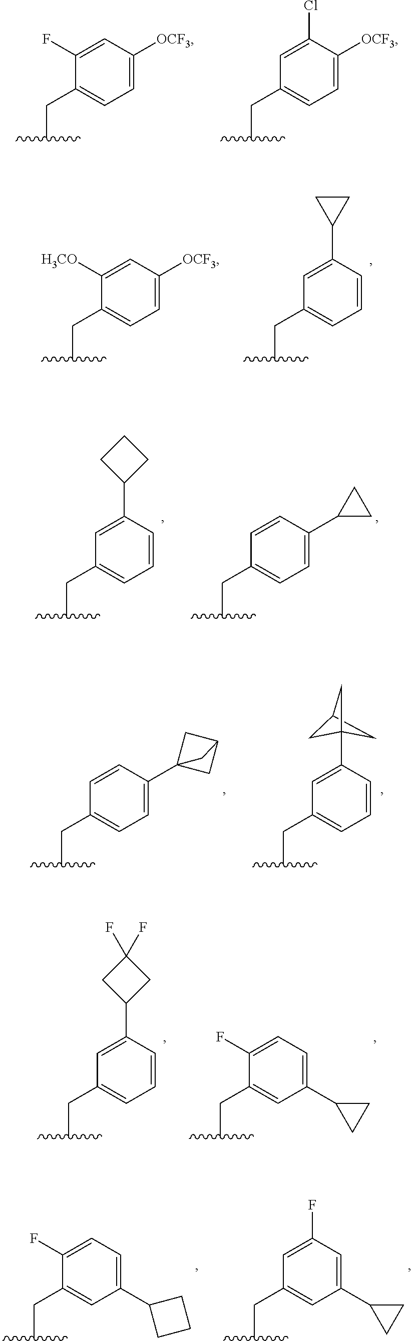

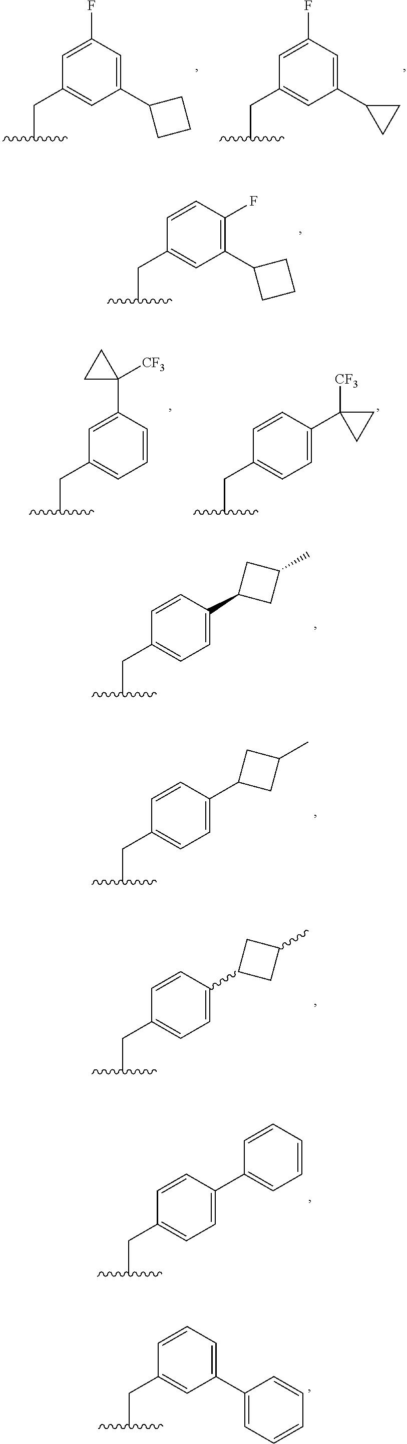

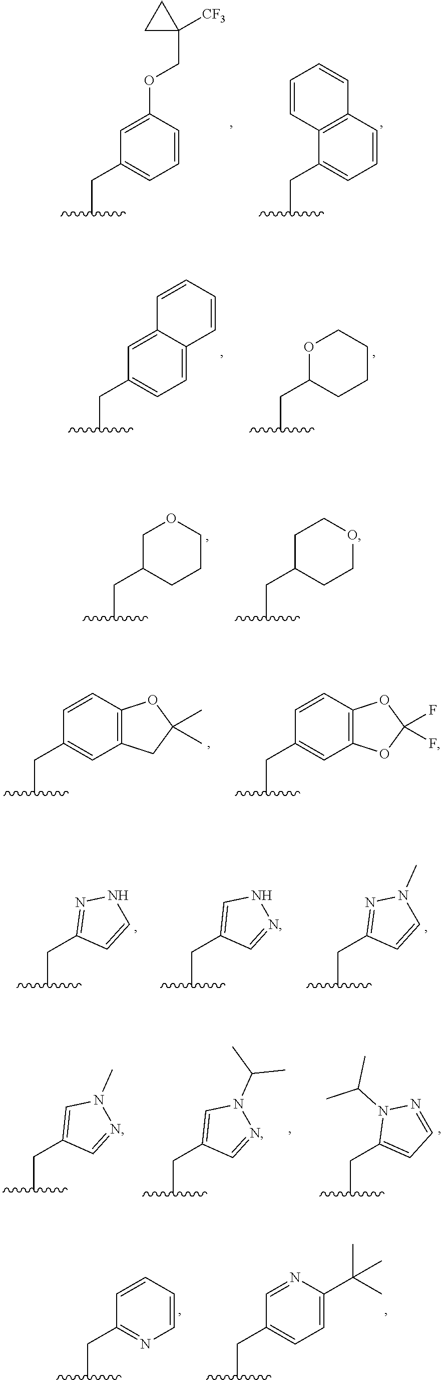

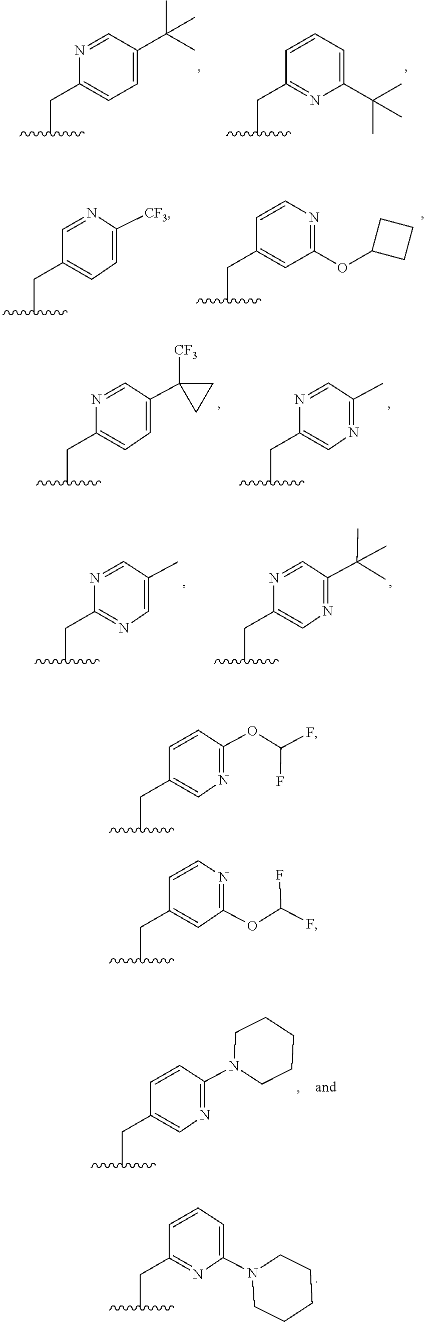

12. The compound, deuterated derivative, or pharmaceutically acceptable salt according to any one of claims 1-11, wherein Ring B is selected from:
C₆-C₁₀ aryl (optionally substituted with 1-3 groups independently selected from halogen),
C₃-C₈ cycloalkyl, and
3- to 6-membered heterocyclyl (optionally substituted with 1-3 groups independently selected from C₁-C₆ alkyl).

13. The compound, deuterated derivative, or pharmaceutically acceptable salt according to any one of claims 1-12, wherein Ring B is selected from.

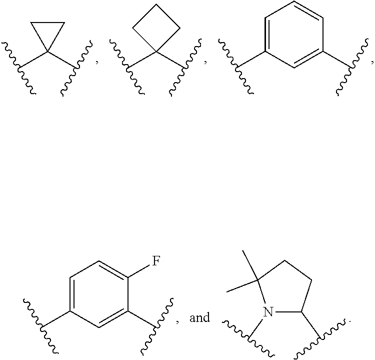

14. The compound, deuterated derivative, or pharmaceutically acceptable salt according to any one of claims 1-13, wherein each R² is H.

15. The compound, deuterated derivative, or pharmaceutically acceptable salt according to any one of claims 1-14, wherein each R¹ is independently selected from C₁-C₆ fluoroalkyl, —N(R²)₂, and —CN.

16. The compound, deuterated derivative, or pharmaceutically acceptable salt according to any one of claims 1-15, wherein each R¹ is independently selected from —CF₃, —NH₂, and —CN.

17. The compound, deuterated derivative, or pharmaceutically acceptable salt according to any one of claims 1-16, wherein R^{Z1} is selected from C₁-C₆ fluoroalkyl.

18. The compound, deuterated derivative, or pharmaceutically acceptable salt according to any one of claims 1-17, wherein R^{Z2} is selected from halogen and hydroxy.

19. The compound, deuterated derivative, or pharmaceutically acceptable salt according to any one of claims 1-18, wherein Z is selected from

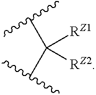

20. The compound, deuterated derivative, or pharmaceutically acceptable salt according to any one of claims 1-19, wherein Z is selected from:

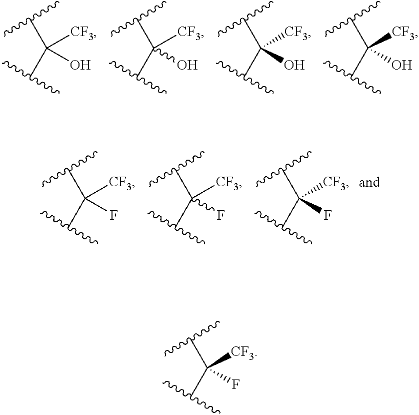

21. The compound, deuterated derivative, or pharmaceutically acceptable salt according to any one of claims 1-20, wherein n is selected from 5, 6, and 7.

22. The compound, deuterated derivative, or pharmaceutically acceptable salt according to any one of claim 1-21, wherein
X is selected from —CH₂— and —CO—;
each Y is independently selected from —C(R^{Y})₂—, —NR^{YN}—, and 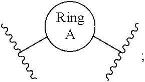
each R^{Y} is independently selected from hydrogen, halogen, and C₁-C₆ alkyl;
each R^{YN} is independently selected from:
C₁-C₄ alkyl, optionally substituted with 1-3 groups independently selected from:
C₆-C₁₀ aryl, optionally substituted with 1-3 groups independently selected from:
C₁-C₆ alkyl,
C₁-C₆ fluoroalkoxy, and
C₃-C₈ cycloalkyl (optionally substituted with 1-3 groups independently selected from C₁-C₆ fluoroalkyl), and
C₃-C₈ cycloalkyl optionally substituted with 1-3 groups independently selected from halogen,
Ring B is selected from C₃-C₈ cycloalkyl,
each R¹ is independently selected from C₁-C₆ fluoroalkyl and —NH₂;
Z is selected from 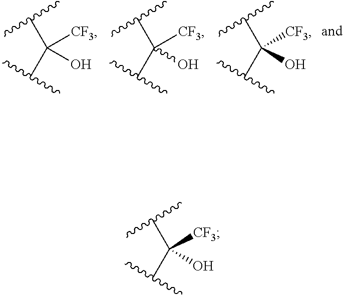
R^{Z1} is selected from C₁-C₆ fluoroalkyl;
R^{Z2} is hydroxy;
n is 6; and
m is 2.

23. The compound, deuterated derivative, or pharmaceutically acceptable salt according to any one of claims 1-22, wherein each —C(R^{Y})₂— is independently selected from: —CH₂—,

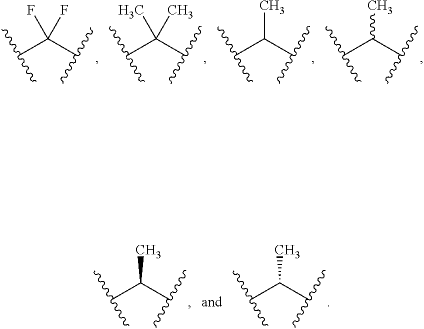

24. The compound, deuterated derivative, or pharmaceutically acceptable salt according to any one of claims 1-23, wherein each wherein each —NR^{YN}— is independently selected from:

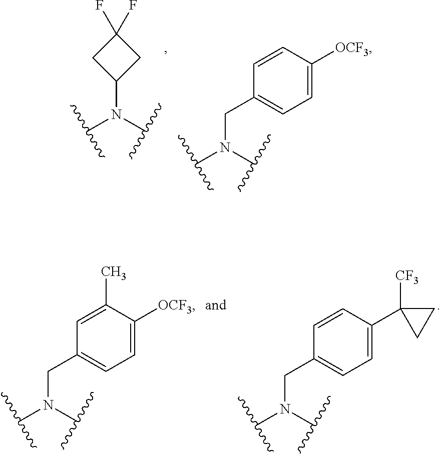 [SMILES: *N(*)C1CC(F)(F)C1 ; *N(*)Cc1ccc(OC(F)(F)F)cc1 ; *N(*)Cc1ccc(OC(F)(F)F)c(C)c1 ; *N(*)Cc1ccc(C2(C(F)(F)F)CC2)cc1]

25. The compound, deuterated derivative, or pharmaceutically acceptable salt according to any one of claims 1-24, wherein Ring B is

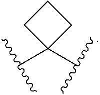 [SMILES: *C1(*)CCC1]

26. The compound, deuterated derivative, or pharmaceutically acceptable salt according to any one of claims 1-25, wherein —(Y)ₙ— is a group selected from:

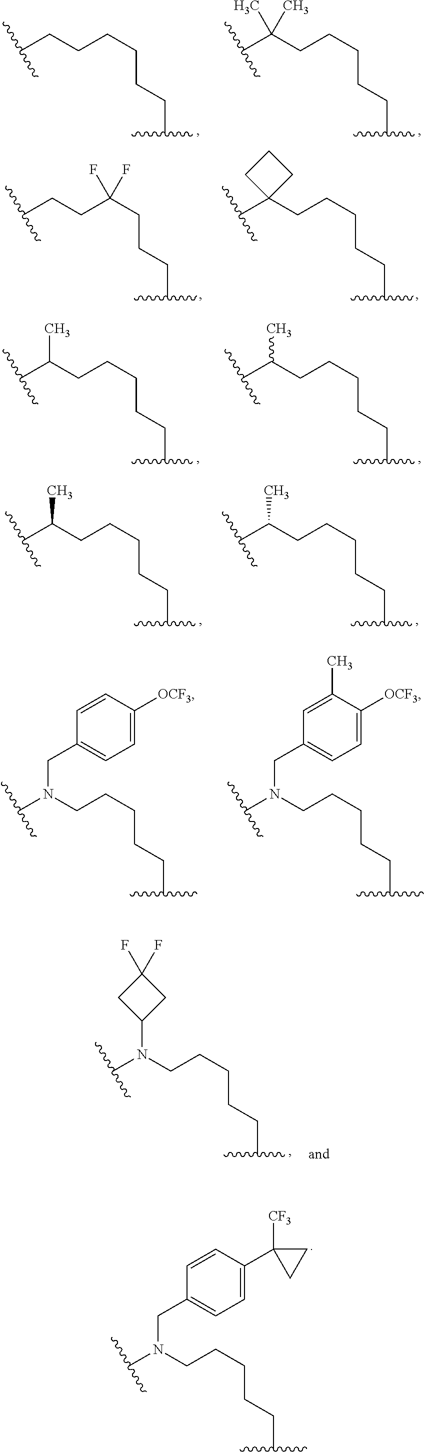

27. The compound, deuterated derivative, or pharmaceutically acceptable salt according to claim 1, wherein the compound is selected from Formula Ta: 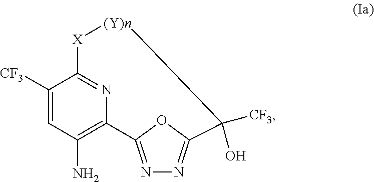
and deuterated derivatives and pharmaceutically acceptable salts thereof, wherein:
X is selected from —CH₂— and —CO—;
n is selected from 5, 6, and 7; 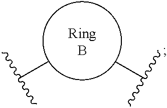
each Y is independently selected from —C(R^{Y})₂—, —NR^{YN}—, and
each R^{Y} is independently selected from hydrogen, hydroxy, halogen, and C₁-C₆ alkyl (optionally substituted with 1-3 groups independently selected from hydroxy, and C₁-C₆ alkoxy);
each R^{YN} is independently selected from:
H,
C₁-C₄ alkyl, optionally substituted with 1-3 groups independently selected from:
oxo,
C₃-C₈ cycloalkyl (optionally substituted with 1-3 groups independently selected from halogen and C₁-C₆ alkyl),
C₆-C₁₀ aryl, optionally substituted with 1-3 groups independently selected from:
halogen,
cyano,
C₁-C₆ alkyl (optionally substituted with 1-3 hydroxy),
C₁-C₆ fluoroalkyl,
C₁-C₆ alkoxy (optionally substituted with C₃-C₈ cycloalkyl, which is optionally substituted with C₁-C₆ fluoroalkyl),
C₁-C₆ fluoroalkoxy,
C₃-C₈ cycloalkyl (optionally substituted with 1-3 groups independently selected from halogen, C₁-C₆ alkyl, and C₁-C₆ fluoroalkyl), and
phenyl (optionally substituted with C₁-C₆ alkyl),
5- to 6-membered heteroaryl, optionally substituted with 1-3 groups independently selected from:
halogen,
C₁-C₆ alkyl,
C₁-C₆ fluoroalkyl,
C₁-C₆ fluoroalkoxy,
—O₍₀₋₁₎(C₃-C₈ cycloalkyl) (optionally substituted with C₁-C₆ fluoroalkyl), and
3- to 12-membered heterocyclyl, and
3- to 12-membered heterocyclyl, optionally substituted with 1-3 groups selected from halogen and C₁-C₆ alkyl)
C₁-C₆ fluoroalkyl,
C₆-C₁₀ aryl,
C₃-C₈ cycloalkyl optionally substituted with 1-3 groups independently selected from
halogen,
cyano,
C₁-C₄ alkyl optionally substituted with 1-3 groups selected from —N(R^{YN1})₂,
C₁-C₆ fluoroalkyl,
C₁-C₆ alkoxy,
C₃-C₈ cycloalkyl, and
phenyl (optionally substituted with 1-3 groups independently selected from C₁-C₆ alkyl)
3- to 12-membered heterocyclyl, optionally substituted with 1-3 groups selected from oxo and C₁-C₄ alkyl (optionally substituted with 1-2 groups selected from oxo and C₃-C₈ cycloalkyl),
5- to 6-membered heteroaryl (optionally substituted with 1-3 groups selected from C₁-C₆ alkyl), and
CO₂R^{YN1},
each R^{YN}1 is independently selected from H, and C₁-C₄ alkyl (optionally substituted with oxo);
Ring B is selected from:
C₆-C₁₀ aryl (optionally substituted with 1-3 groups independently selected from halogen, C₁-C₆ alkyl, and C₁-C₆ alkoxy),
C₃-C₈ cycloalkyl, and
3- to 6-membered heterocyclyl (optionally substituted with 1-3 groups independently selected from C₁-C₆ alkyl);
each R¹ is independently selected from halogen, C₁-C₆ fluoroalkyl, C₁-C₆ alkyl, —N(R²)₂, and —CN; and
each R² is independently selected from hydrogen, C₁-C₃ alkyl, and C₁-C₆ fluoroalkyl.

28. The compound, deuterated derivative, or pharmaceutically acceptable salt according to claim 1, wherein the compound is selected from Formula Ia′: 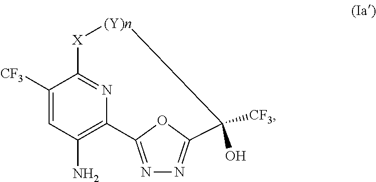
and deuterated derivatives and pharmaceutically acceptable salts thereof, wherein:
X is selected from —CH₂— and —CO—;
n is selected from 5, 6, and 7;
each Y is independently selected from —C(R^{Y})₂—, —NR^{YN}—, and 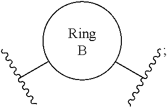
each R^{Y} is independently selected from hydrogen, hydroxy, halogen, and C₁-C₆ alkyl (optionally substituted with 1-3 groups independently selected from hydroxy, and C₁-C₆ alkoxy);
each R^{YN} is independently selected from:
H,
C₁-C₄ alkyl, optionally substituted with 1-3 groups independently selected from:
oxo,
C₃-C₈ cycloalkyl (optionally substituted with 1-3 groups independently selected from halogen and C₁-C₆ alkyl),
C₆-C₁₀ aryl, optionally substituted with 1-3 groups independently selected from:
halogen,
cyano,
C₁-C₆ alkyl (optionally substituted with 1-3 hydroxy),
C₁-C₆ fluoroalkyl,
C₁-C₆ alkoxy (optionally substituted with C₃-C₈ cycloalkyl, which is optionally substituted with C₁-C₆ fluoroalkyl),
C₁-C₆ fluoroalkoxy,
C₃-C₈ cycloalkyl (optionally substituted with 1-3 groups independently selected from halogen, C₁-C₆ alkyl, and C₁-C₆ fluoroalkyl), and
phenyl (optionally substituted with C₁-C₆ alkyl),
5- to 6-membered heteroaryl, optionally substituted with 1-3 groups independently selected from:
halogen,
C₁-C₆ alkyl,
C₁-C₆ fluoroalkyl,
C₁-C₆ fluoroalkoxy,
—O₍₀₋₁₎(C₃-C₈ cycloalkyl) (optionally substituted with C₁-C₆ fluoroalkyl), and
3- to 12-membered heterocyclyl, and
3- to 12-membered heterocyclyl, optionally substituted with 1-3 groups selected from halogen and C₁-C₆ alkyl)
C₁-C₆ fluoroalkyl,
C₆-C₁₀ aryl,
C₃-C₈ cycloalkyl optionally substituted with 1-3 groups independently selected from
halogen,
cyano,
C₁-C₄ alkyl optionally substituted with 1-3 groups selected from —N(R^{YN1})₂,
C₁-C₆ fluoroalkyl,
C₁-C₆ alkoxy,
C₃-C₈ cycloalkyl, and
phenyl (optionally substituted with 1-3 groups independently selected from C₁-C₆ alkyl)
3- to 12-membered heterocyclyl, optionally substituted with 1-3 groups selected from oxo and C₁-C₄ alkyl (optionally substituted with 1-2 groups selected from oxo and C₃-C₈ cycloalkyl),
5- to 6-membered heteroaryl (optionally substituted with 1-3 groups selected from C₁-C₆ alkyl), and
CO₂R^{YN1},
each R^{YN}1 is independently selected from H, and C₁-C₄ alkyl (optionally substituted with oxo);
Ring B is selected from:
C₆-C₁₀ aryl (optionally substituted with 1-3 groups independently selected from halogen, C₁-C₆ alkyl, and C₁-C₆ alkoxy),
C₃-C₈ cycloalkyl, and
3- to 6-membered heterocyclyl (optionally substituted with 1-3 groups independently selected from C₁-C₆ alkyl);
each R¹ is independently selected from halogen, C₁-C₆ fluoroalkyl, C₁-C₆ alkyl, —N(R²)₂, and —CN; and
each R² is independently selected from hydrogen, C₁-C₃ alkyl, and C₁-C₆ fluoroalkyl.

29. The compound, deuterated derivative, or pharmaceutically acceptable salt of claim 27 or claim 28, wherein n is 6.

30. The compound, deuterated derivative, or pharmaceutically acceptable salt according to claim 1, wherein the compound is selected from Formula Ib: 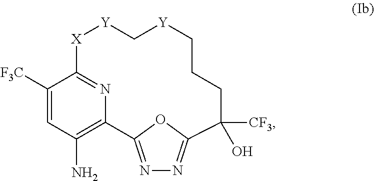
and deuterated derivatives and pharmaceutically acceptable salts thereof, wherein:
X is selected from —CH₂— and —CO—; and
each Y is independently selected from —CH₂—, —C(CH₃)₂—, —CH(CH₃)—, —CF₂—, cyclobutyl, and —NR^{YN}—, wherein each R^{YN} is independently selected from:
C₁-C₄ alkyl substituted with phenyl, which is optionally substituted with 1-3 groups independently selected from C₁-C₆ alkyl, C₁-C₆ fluoroalkoxy, and C₃-C₈ cycloalkyl (optionally substituted with 1-3 groups independently selected from C₁-C₆ fluoroalkyl), and
C₃-C₈ cycloalkyl optionally substituted with 1-3 groups independently selected from halogen.

31. The compound, deuterated derivative, or pharmaceutically acceptable salt according to claim 1, wherein the compound is selected from Formula Ib′: 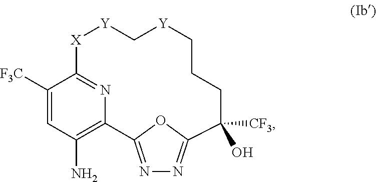
and deuterated derivatives and pharmaceutically acceptable salts thereof, wherein:
X is selected from —CH₂— and —CO—; and
each Y is independently selected from —CH₂—, —C(CH₃)₂—, —CH(CH₃)—, —CF₂—, cyclobutyl, and —NR^{YN}—, wherein each R^{YN} is independently selected from:
C₁-C₄ alkyl substituted with phenyl, which is optionally substituted with 1-3 groups independently selected from C₁-C₆ alkyl, C₁-C₆ fluoroalkoxy, and C₃-C₈ cycloalkyl (optionally substituted with 1-3 groups independently selected from C₁-C₆ fluoroalkyl), and
C₃-C₈ cycloalkyl optionally substituted with 1-3 groups independently selected from halogen.

32. The compound, deuterated derivative, or pharmaceutically acceptable salt of any one of claims 27 to 31, wherein X is —CO—.

33. The compound, deuterated derivative, or pharmaceutically acceptable salt of any one of claims 27 to 31, wherein X is —CH₂—.

34. A compound selected from compounds of Table 7, pharmaceutically acceptable salts thereof, and deuterated derivatives of any of the foregoing.

35. A compound according to any one of claims 1-31 or claim 34, wherein the compound is selected from:

| Comp. No. | Structure |
|---|---|
| 48 | 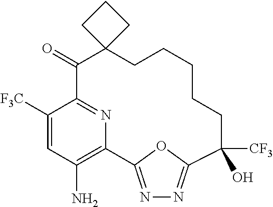 [SMILES: Nc1cc(C(F)(F)F)c2nc1-c1nnc(o1)[C@@](O)(C(F)(F)F)CCCCCC1(CCC1)C2=O] |
| 45 | 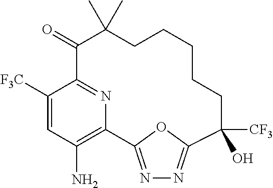 [SMILES: CC1(C)CCCCC[C@](O)(C(F)(F)F)c2nnc(o2)-c2nc(c(C(F)(F)F)cc2N)C1=O] |
| 2 | 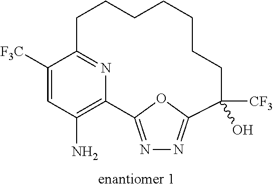 [SMILES: Nc1cc(C(F)(F)F)c2nc1-c1nnc(o1)C(O)(C(F)(F)F)CCCCCCC2] |
| 165 | 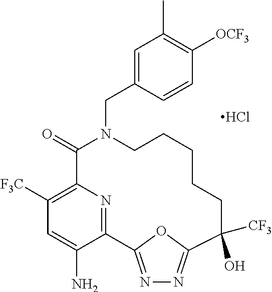 [SMILES: Cc1cc(CN2CCCCC[C@](O)(C(F)(F)F)c3nnc(o3)-c3nc(c(C(F)(F)F)cc3N)C2=O)ccc1OC(F)(F)F ; Cl] |
| 151 | 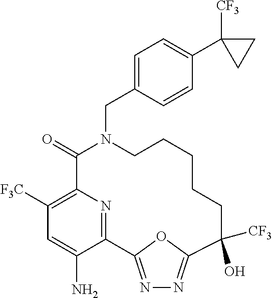 [SMILES: Nc1cc(C(F)(F)F)c2nc1-c1nnc(o1)[C@@](O)(C(F)(F)F)CCCCCN(Cc1ccc(C3(C(F)(F)F)CC3)cc1)C2=O] |
| 6 | 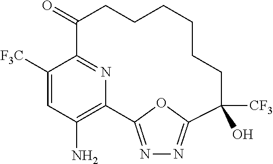 [SMILES: Nc1cc(C(F)(F)F)c2nc1-c1nnc(o1)[C@@](O)(C(F)(F)F)CCCCCCC2=O] |
| 106 | 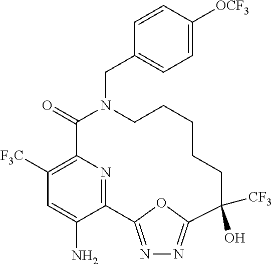 [SMILES: Nc1cc(C(F)(F)F)c2nc1-c1nnc(o1)[C@@](O)(C(F)(F)F)CCCCCN(Cc1ccc(OC(F)(F)F)cc1)C2=O] |
| 84 | 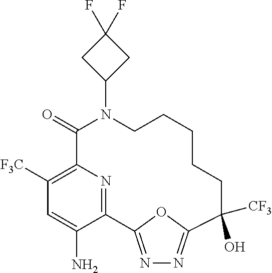 [SMILES: Nc1cc(C(F)(F)F)c2nc1-c1nnc(o1)[C@@](O)(C(F)(F)F)CCCCCN(C1CC(F)(F)C1)C2=O] |
| 38 | 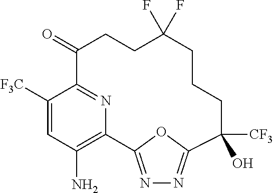 [SMILES: Nc1cc(C(F)(F)F)c2nc1-c1nnc(o1)[C@@](O)(C(F)(F)F)CCCC(F)(F)CCC2=O] |
| 50 | 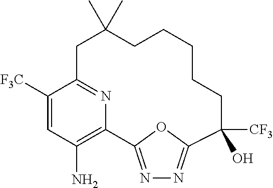 [SMILES: CC1(C)CCCCC[C@](O)(C(F)(F)F)c2nnc(o2)-c2nc(c(C(F)(F)F)cc2N)C1] |
| 56 | 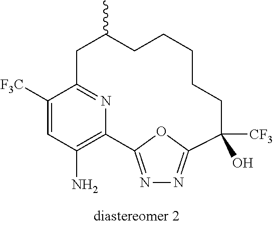 [SMILES: CC1CCCCC[C@](O)(C(F)(F)F)c2nnc(o2)-c2nc(c(C(F)(F)F)cc2N)C1] |
| 55 | 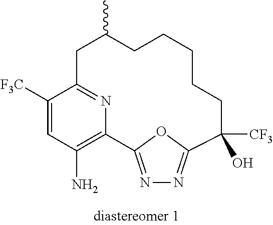 [SMILES: CC1CCCCC[C@](O)(C(F)(F)F)c2nnc(o2)-c2nc(c(C(F)(F)F)cc2N)C1] |

36. A pharmaceutical composition comprising a compound, deuterated derivative, or pharmaceutically acceptable salt of any one of claims 1-35 and a pharmaceutically acceptable carrier.

37. The pharmaceutical composition according to claim 36, further comprising one or more additional therapeutic agent(s).

38. The pharmaceutical composition according to claim 37, wherein the one or more additional therapeutic agent(s) comprise(s) a compound with CFTR modulating activity or a salt or deuterated derivative thereof.

39. The pharmaceutical composition according to claim 37 or claim 38, wherein the one or more additional therapeutic agent(s) comprise(s) one or more compounds selected from:
(a) (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound II): 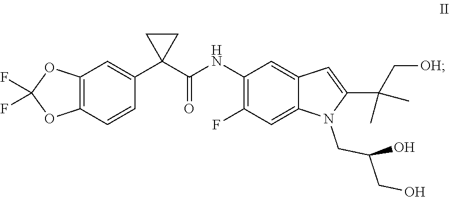 [SMILES: CC(C)(CO)c1cc2cc(NC(=O)C3(c4ccc5c(c4)OC(F)(F)O5)CC3)c(F)cc2n1C[C@@H](O)CO]
(b) 3-(6-(1-(2,2-difluorobenzo[d][1,3]dioxol-5-yl)cyclopropanecarboxamido)-3-methylpyridin-2-yl)benzoic acid (Compound IV): 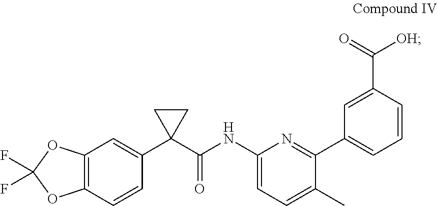 [SMILES: Cc1ccc(NC(=O)C2(c3ccc4c(c3)OC(F)(F)O4)CC2)nc1-c1cccc(C(=O)O)c1]
(c) N-(1,3-dimethylpyrazol-4-yl)sulfonyl-6-[3-(3,3,3-trifluoro-2,2-dimethyl-propoxy)pyrazol-1-yl]-2-[(4S)-2,2,4-trimethylpyrrolidin-1-yl]pyridine-3-carboxamide (Compound V): 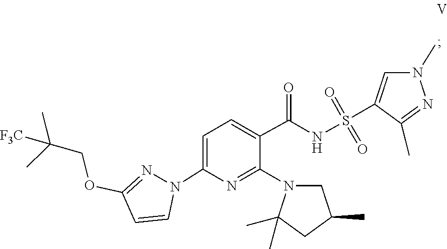 [SMILES: Cc1nn(C)cc1S(=O)(=O)NC(=O)c1ccc(-n2ccc(OCC(C)(C)C(F)(F)F)n2)nc1N1C[C@@H](C)CC1(C)C]
(d) N-(benzenesulfonyl)-6-[3-[2-[1-(trifluoromethyl)cyclopropyl]ethoxy]pyrazol-1-yl]-2-[(4S)-2,2,4-trimethylpyrrolidin-1-yl]pyridine-3-carboxamide (Compound VI): 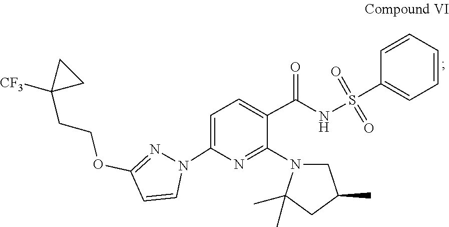 [SMILES: C[C@@H]1CN(c2nc(-n3ccc(OCCC4(C(F)(F)F)CC4)n3)ccc2C(=O)NS(=O)(=O)c2ccccc2)C(C)(C)C1]
(e) (14S)-8-[3-(2-{dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione (Compound VII): 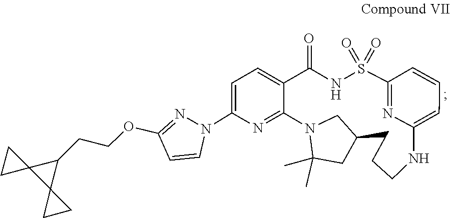 [SMILES: CC1(C)C[C@@H]2CCCNc3cccc(n3)S(=O)(=O)NC(=O)c3ccc(-n4ccc(OCCC5C6(CC6)C56CC6)n4)nc3N1C2]
and
(f) (11R)-6-(2,6-dimethylphenyl)-11-(2-methylpropyl)-12-{spiro[2.3]hexan-5-yl}-9-oxa-2λ⁶-thia-3,5,12,19-tetraazatricyclo[12.3.1.14,8]nonadeca-1(17),4(19),5,7,14(18),15-hexaene-2,2,13-trione (Compound VIII):

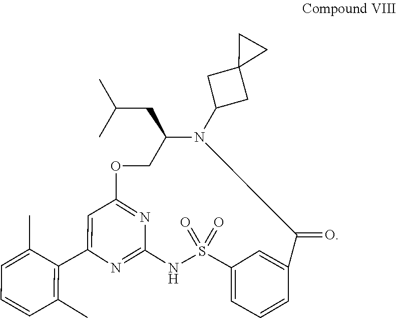 [SMILES: Cc1cccc(C)c1-c1cc2nc(n1)NS(=O)(=O)c1cccc(c1)C(=O)N(C1CC3(CC3)C1)[C@H](CC(C)C)CO2]

40. The pharmaceutical composition according to any one of claims 37-39, wherein the one or more additional therapeutic agent(s) comprise(s) a compound selected from N-(5-hydroxy-2,4-di-tert-butyl-phenyl)-4-oxo-1H-quinoline-3-carboxamide (Compound III): 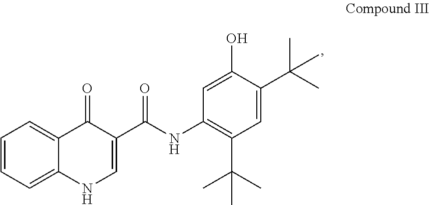 [SMILES: CC(C)(C)c1cc(C(C)(C)C)c(NC(=O)c2c[nH]c3ccccc3c2=O)cc1O]
and N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound III-d):

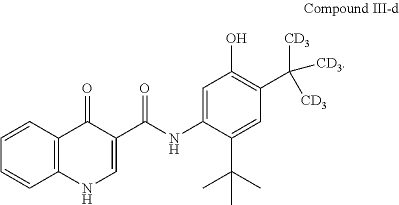 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

41. A method of treating cystic fibrosis, comprising administering an effective amount of the compound, salt, or deuterated derivative according to any one of claims 1-35 or the pharmaceutical composition according to any one of claims 36-40 to a patient in need thereof.

42. The method according to claim 41, further comprising administering one or more additional therapeutic agent(s).

43. The method according to claim 42, wherein the one or more additional therapeutic agent(s) comprise(s) a compound with CFTR modulating activity or a salt or deuterated derivative thereof.

44. The method according to claim 42 or claim 43, wherein the one or more additional therapeutic agent(s) comprise(s) one or more compounds selected from:
(a) Compound II,
(b) Compound IV,
(c) Compound V,
(d) Compound VI,
(e) Compound VII, and
(f) Compound VIII:

45. The method according to any one of claims 42-44, wherein the one or more additional therapeutic agent(s) comprise(s) a compound selected from Compound III and Compound III-d.

46. The compound, salt, or deuterated derivative of any one of claims 1-35 or the pharmaceutical composition according to any one of claims 36-40 for use in the treatment of cystic fibrosis.

47. Use of the compound, salt, or deuterated derivative of any one of claims 1-35 or the pharmaceutical composition according to any one of claims 36-40 in the manufacture of a medicament for the treatment of cystic fibrosis.
